(19) United States
(12) Patent Application Publication
(10) Pub. No.: US 2023/0099344 A1
(43) Pub. Date: Mar. 30, 2023

(54) COMPOUNDS AND METHODS FOR THE TARGETED DEGRADATION OF IRAK-4

(71) Applicant: Arvinas Operations, Inc., NEW HAVEN, CT (US)

(72) Inventors: Erika Marina Vieira ARAUJO, Woodbridge, CT (US); Jennifer L. CANTLEY, GUILFORD, CT (US); Keith R. HORNBERGER, SOUTHBURY, CT (US); Jesus Raul MEDINA, Gilbertsville, PA (US)

(21) Appl. No.: 17/807,094

(22) Filed: Jun. 15, 2022

Related U.S. Application Data
(60) Provisional application No. 63/210,880, filed on Jun. 15, 2021.

(51) Int. Cl. A61K 47/55; A61K 47/54; A61K 45/06; A61P 35/00

(52) U.S. Cl. CPC A61K 47/55; A61K 47/545; A61K 45/06; A61P 35/00

(57) ABSTRACT

This application relates to a bifunctional compound having the structure of Formula (I):

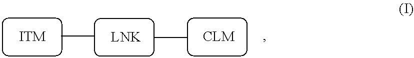

or a pharmaceutically acceptable salt, solvate, enantiomer, stereoisomer, or isotopic derivative thereof, which contains, on one end, a cereblon E3 ubiquitin ligase that binds to the E3 ubiquitin ligase and, on the other end, a moiety that binds the target protein, Interleukin-1 Receptor-Associated Kinase 4 (IRAK-4), such that the target protein is placed in proximity to the ubiquitin ligase to effect degradation (and inhibition) of target protein. The bifunctional compounds of the present disclosure present disclosure exhibit a broad range of pharmacological activities associated with degradation/inhibition of target protein. Diseases or disorders that result from cellular signaling mediated by the target protein are treated or prevented with bifunctional compounds and compositions of the present disclosure.

### RELATED APPLICATION

This application claims priority to U.S. provisional application No. 63/210,880, filed Jun. 15, 2021, the contents of which are incorporated herein in their entirety.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore are more attractive therapeutic targets than general proteasome inhibitors due to their specificity for certain protein substrates. The development of ligands of E3 ligases has proven challenging, in part because they must disrupt protein-protein interactions. However, notable developments have provided specific ligands which bind to these ligases. For example, since the discovery of nutlins, the first small molecule E3 ligase inhibitors, additional compounds have been reported that target E3 ligases.

Cereblon is a protein that in humans is encoded by the CRBN gene. CRBN orthologs are highly conserved from plants to humans, which underscores its physiological importance. Cereblon forms an E3 ubiquitin ligase complex with damaged DNA binding protein 1 (DDB1), Cullin-4A (CUL4A), and regulator of cullins 1 (ROC 1). This complex ubiquitinates several other proteins. Through a mechanism which has not been completely elucidated, cereblon ubiquitination of target proteins results in increased levels of fibroblast growth factor 8 (FGF8) and fibroblast growth factor 10 (FGF10). FGF8 in turn regulates several developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

Interleukin-1 (IL-1) Receptor-Associated Kinase-4 (IRAK-4) is a serine/threonine kinase enzyme that plays an essential role in signal transduction by Toll/IL-1 receptors (TIRs). Diverse IRAK enzymes are key components in the signal transduction pathways mediated by interleukin-1 receptor (IL-1R) and Toll-like receptors (TLRs) (Janssens, S, et al. Mol. Cell. 11(2), 2003, 293-302). There are four members in the mammalian IRAK family: IRAK-1, IRAK-2, IRAK-3, and IRAK-4. These proteins are characterized by a typical N-terminal death domain that mediates interaction with MyD88-family adaptor proteins and a centrally located kinase domain. Upon recruitment of MyD88 and IRAK-4 to the Toll-interleukin domain, activation leads to phosphorylation of IRAK-1 and/or IRAK-2 leading to engagement of TRAF6. Subsequent signaling results in activation of NF-κB and the release of various cytokines and chemokines. The MYD88 L265P mutation occurring in 91% Waldenstrom's macroglobulinemia, 29% ABC DLBCL, 9% MALT lymphomas, and 3% CLL coordinates a constitutively active signalosome in which IRAK-4-mediated phosphorylation of IRAK-1 promotes the assembly of additional signaling proteins driving survival in these cancers.

The IRAK proteins, as well as MyD88, have been shown to play a role in transducing signals other than those originating from IL-1R receptors, including signals triggered by activation of IL-18 receptors (Kanakaraj, et al. J. Exp. Med. 189(7), 1999, 1129-38) and LPS receptors (Yang, et al., J. Immunol. 163(2), 1999, 639-43). Out of the four members in the mammalian IRAK family, IRAK-4 is the “master IRAK”. Under overexpression conditions, all IRAKs can mediate the activation of nuclear factor-KB (NF-κB) and stress-induced mitogen activated protein kinase (MAPK)-signaling cascades. However, only IRAK-1 and IRAK-4 have been shown to have active kinase activity. While IRAK-1 kinase activity could be dispensable for its function in IL-1-induced NF-κB activation (Kanakaraj et al, J. Exp. Med. 187(12), 1998, 2073-9; and Li, et al. Mol. Cell. Biol. 19(7), 1999, 4643-52), IRAK-4 requires its kinase activity for signal transduction (Li S, et al. Proc. Natl. Acad. Sci. USA 99(8), 2002, 5567-72; and Lye, E. et al, J. Biol. Chem. 279(39); 2004, 40653-8). Given the central role of IRAK-4 in Toll-like/IL-1R signaling and immunological protection, IRAK-4 inhibitors have been identified as potentially valuable therapeutics in inflammatory diseases, sepsis and autoimmune disorders (Wietek C, et al, Mol. Interv. 2: 2002, 212-5). Since IRAK-4 possesses scaffolding as well as kinase-dependent signaling activity (Lee K L, Ambler C M, Anderson D R, et al. J Med Chem. 2017; 60(13):5521-42), degradation of IRAK-4 may be a valuable alternative to inhibition by small molecules.

Mice lacking IRAK-4 are viable and show complete abrogation of inflammatory cytokine production in response to IL-1, IL-18 or LPS (Suzuki et al. Nature, 416(6882), 2002, 750-6). Human patients lacking IRAK-4 are severely immunocompromised and are not responsive to these cytokines (Medvedev et al. J. Exp. Med., 198(4), 2003, 521-31; and Picard et al. Science 299(5615), 2003, 2076-9). Knock-in mice containing inactive IRAK-4 were completely resistant to lipopolysaccharide- and CpG-induced shock (Kim T W, et al. J. Exp. Med 204(5), 2007, 1025-36; and Kawagoe T, et al. J. Exp. Med. 204(5): 2007, 1013-24) and illustrated that IRAK-4 kinase activity is essential for cytokine production, activation of MAPKs and induction of NF-κB regulated genes in response to TLR ligands (Koziczak-Holbro M, et al. J. Biol. Chem. 282(18): 2007, 13552-60). Inactivation of IRAK-4 kinase (IRAK-4 KI) in mice leads to resistance to EAE due to reduction in infiltrating inflammatory cells into CNS and reduced antigen specific CD4+ T-cell mediated IL-17 production (Staschke et al. The Journal of Immunology, 183(1), 2009, 568-77).

Bifunctional compounds, such as those described in U.S. Patent Application Publication Nos. 2015/0291562 and 2014/0356322 (both incorporated herein by reference), function to recruit endogenous proteins to an E3 ubiquitin ligase for ubiquitination and degradation. In particular, the publications describe bifunctional or proteolysis targeting chimeric compounds (PROTAC® protein degraders), which find utility as modulators of targeted ubiquitination of a variety of polypeptides and proteins, which are then degraded via the proteasome system.

An ongoing need exists in the art for effective treatments for disease associated signaling cascades mediated by IRAK-4. However, non-specific effects, and the inability to effectively target both the kinase and scaffolding roles of IRAK-4, remain as obstacles to the development of effective treatments. As such, small-molecule therapeutic agents that target IRAK-4 and that leverage protein degradation via the ubiquitin proteasome system would be useful.

### SUMMARY

The present disclosure describes bifunctional compounds which function to recruit endogenous proteins to an E3 ubiquitin ligase for ubiquitination and degradation, and methods of using the same. In particular, the present disclosure provides bifunctional or proteolysis targeting chimeric compounds (PROTAC® protein degraders), which find utility as modulators of targeted ubiquitination of a variety of polypeptides and proteins (e.g., IRAK-4), which are then degraded and/or otherwise inhibited by the bifunctional compounds described herein. In addition, the description provides methods of using an effective amount of the compounds described herein for the treatment or amelioration of a disease condition, such as cancer, inflammatory diseases/disorders, neurodegenerative diseases, as well as cardiovascular diseases/disorders.

In one aspect, the present disclosure relates to bifunctional compounds having the structure of Formula (Ia):

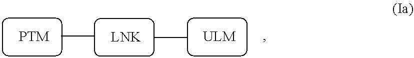

or a pharmaceutically acceptable salt, solvate, enantiomer, stereoisomer, or isotopic derivative thereof, wherein PTM is a protein/polypeptide targeting moiety, LNK is a linker, e.g., a bond (absent) or a chemical group coupling PTM to ULM, and ULM is an E3 ubiquitin ligase binding moiety. The PTM binds to a target protein or polypeptide, which is to be ubiquitinated by an ubiquitin ligase and is chemically linked directly to the ULM group or through a linker moiety LNK.

In one aspect, this application pertains to a bifunctional compound having the structure of Formula (I):

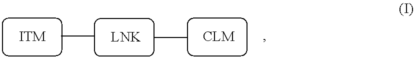

or a pharmaceutically acceptable salt, solvate, enantiomer, stereoisomer, or isotopic derivative thereof,
wherein:
   (a) ITM is an Interleukin-1 Receptor-Associated Kinase 4 (IRAK-4) targeting moiety having the structure of formula ITM-I:

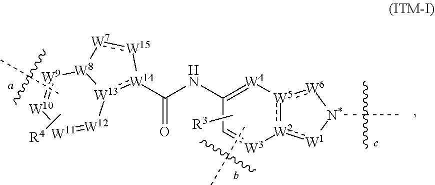

wherein [symbol]
   is a single bond or a double bond;
N* is N or NH
W¹ and W⁶ are each independently C═O, CH, O, N, CH₂, CR¹, or NR¹;
W², W³, W⁴, W⁵, W⁷, W⁸, W⁹, W¹⁰, W¹¹, W¹², W¹³, W¹⁴, and W¹⁵ are each independently C, N, C(R¹)ₘ, or NR¹;
R¹ is H, —Cl, —F, —Br, —I, N(R²)₂; linear or branched C₁₋₆ alkyl, C₁₋₆ haloalkyl, 3- to 7-membered heterocycloalkyl, or 5- to 6-membered heteroaryl optionally substituted with one, two, three, four, or five R⁶;
R³ and R⁴ are each independently selected from the group consisting of H, —Cl, —F, —Br, —I, linear or branched C₁₋₆ alkyl optionally substituted with one, two, three, four, or five R⁷, OC₁₋₆ alkyl, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C₃₋₇ cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, and C₁₋₆alkyl-C₃₋₇cycloalkyl optionally substituted with one, two, three, four, or five R⁷;
R^{N} is H, linear or branched C₁₋₆ alkyl, or C₁₋₆ haloalkyl;
each R² is independently selected from the group consisting of H and linear or branched C₁₋₆ alkyl;
each R⁶ is independently selected from the group consisting of OH, OC₁₋₆ alkyl, and heterocycloalkyl;
each R⁷ is independently selected from the group consisting of —Cl, —F, —Br, —I, NH2, CN, CF₃, linear or branched C₁₋₆ alkyl, OC₁₋₆ alkyl, NH(C1-C₆ alkyl), N(C₁₋₆ alkyl)(C₁₋₆ alkyl), C₆-C₁₀ aryl, 5- to 6-membered heteroaryl, C₃-C₇ cycloalkyl, and 3- to 7-membered heterocycloalkyl;
m is 1 or 2; and
   ITM is connected to LNK through one of

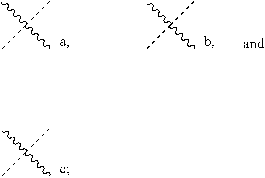

(b) LNK is a chemical linking moiety that covalently couples the ITM to the CLM, having the structure:
[symbol] L [symbol] ₙ
wherein:
each L is independently selected from the group consisting of X, Y, and Z;
each X is independently selected from

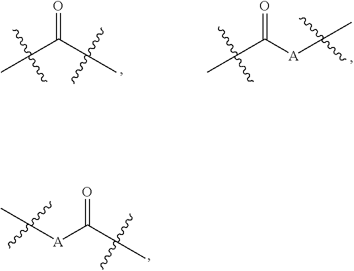

and -A-, wherein each A is independently selected from the group consisting of C(R^{8A})₂, NR⁸, and O;
each R^{8A} is independently selected from the group consisting of H, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, —Cl, —F, —Br, —I, and OH;
   each R⁸ is independently selected from the group consisting of H, linear or branched C₁₋₆ alkyl optionally substituted with —Cl, —F, —OH, NH₂, CN, or CF₃, O—C₁₋₆ alkyl, and C₁₋₆ haloalkyl;
each Y is independently selected from the group consisting of

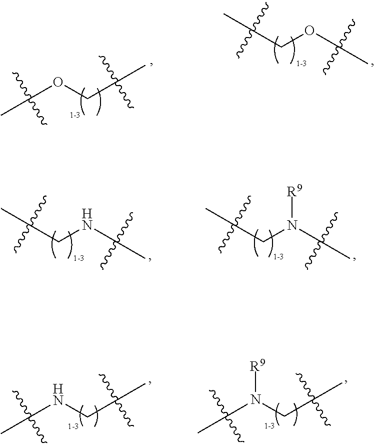

C₁₋₆ alkylene, C₂₋₆ alkenylene, and C₂₋₆ alkynylene;
wherein each Y is independently optionally substituted with one, two, three, four, or five R⁹;
each R⁹ is independently selected from the group consisting of linear or branched C₁₋₆ alkyl optionally substituted with —Cl, —F, —OH, NH₂, CN, or CF₃; O—C₁₋₆ alkyl optionally substituted with —Cl, —F, —OH, NH₂, CN, or CF₃, C₁₋₆ haloalkyl, NH, CN, CF₃, —Cl, —F, —Br, —I, and OH;
each Z is independently selected from the group consisting of monocyclic C₄₋₁₀ cycloalkylene, fused bicyclic C₄₋₁₀ cycloalkylene, bridged bicyclic C₄₋₁₀ cycloalkylene, or spiro-fused bicyclic C₄₋₁₀ cycloalkylene, 4-6 membered heterocycloalkylene, C₆-C₁₀ arylene, and 5-6 membered heteroarylene;
wherein each Z is independently optionally substituted with one, two, three, four, or five R¹⁰;
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH; and
n is 1, 2, 3, 4, 5, or 6;
   (c) CLM is a cereblon E3 ubiquitin ligase binding moiety having a structure selected from the group consisting of formulae CLM-I, CLM-II, and CLM-III:

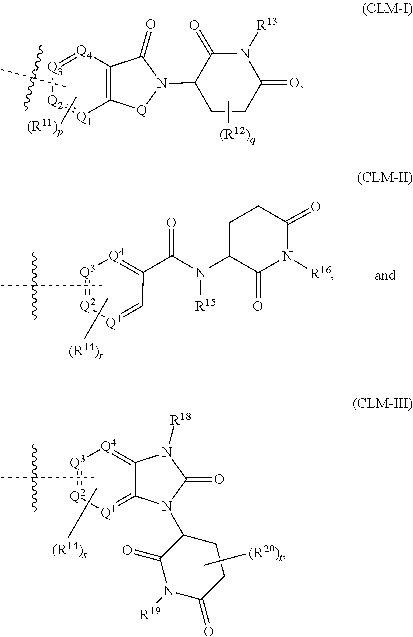

wherein:
Q¹, Q², Q³, and Q⁴ are each independently selected from the group consisting of CH, C, and N;
each R¹¹ is independently selected from the group consisting of —Cl, —F, —Br, —I, CN, CF₃, NH₂, NH—(C1-C₆ alkyl), N(C1-C₆ alkyl)(C1-C₆ alkyl), OH, O—C₁₋₆ alkyl optionally substituted with R²¹, and linear or branched C₁₋₆ alkyl optionally substituted with R²¹;
p is, 1, 2, 3, or 4;
Q is selected from the group consisting of CH₂, CH(C₁₋₆ alkyl), C═O, SO₂, NH, and N(C₁₋₆ alkyl);
each R¹² is independently selected from the group consisting of —Cl, —F, —Br, —I, O—C₁₋₆ alkyl optionally substituted with R²¹, NH₂, NH—(C1-C₆ alkyl), N(C1-C₆ alkyl)(C1-C₆ alkyl), OH, CN, CF₃, and linear or branched C₁₋₆ alkyl optionally substituted with R²¹;
q is 0, 1, 2, or 3;
R¹³ is selected from the group consisting of H, OH, linear or branched C₁₋₆ alkyl optionally substituted with R¹, C(O)C₁₋₆ alkyl, C(O)NH—C₁₋₆ alkyl and C(O)OC₁₋₆ alkyl;
each R¹⁴ is independently selected from the group consisting of —Cl, —F, —Br, —I, NH₂, NH—(C1-C₆ alkyl), N(C1-C₆ alkyl)(C1-C₆ alkyl), OH, CN, CF₃, O—C₁₋₆ alkyl optionally substituted with R²¹, and linear or branched C₁₋₆ alkyl optionally substituted with R²¹;
r is 0, 1, 2, 3, or 4;
R¹⁵ is selected from the group consisting of H and linear or branched C₁₋₆ alkyl optionally substituted with R²¹;
R¹⁶ is selected from the group consisting of H, OH, linear or branched C₁₋₆ alkyl optionally substituted with R²¹, C(O)C₁₋₆ alkyl, C(O)NH—C₁₋₆ alkyl, and C(O)OC₁₋₆ alkyl;
s is 0, 1, 2, or 3;
R¹⁸ is selected from the group consisting of H and linear or branched C₁₋₆ alkyl optionally substituted with R²¹;
R¹⁹ is selected from the group consisting of H, OH, linear or branched C₁₋₆ alkyl optionally substituted with R²¹, C(O)C₁₋₆ alkyl, C(O)NH—C₁₋₆ alkyl, and C(O)OC₁₋₆ alkyl;
each R²⁰ is independently selected from the group consisting of —Cl, —F, —Br, —I, O—C₁₋₆ alkyl optionally substituted with R²¹, NH₂, NH—(C1-C₆ alkyl), N(C1-C₆ alkyl)(C1-C₆ alkyl), OH, CN, CF₃, and linear or branched C₁₋₆ alkyl optionally substituted with R²¹;
t is 0, 1, 2, or 3;
each R²¹ is independently selected from the group consisting of —Cl, —F, —OH, NH₂, CN, and CF₃; and
[symbol] is the point of attachment to LNK.

In another aspect, the present disclosure provides a pharmaceutical composition comprising a bifunctional compound of the present disclosure, or a pharmaceutically acceptable salt, solvate, enantiomer, stereoisomer, or isotopic derivative thereof, and one or more pharmaceutically acceptable excipients. In some embodiments, the pharmaceutical composition further comprises an additional bioactive agent, wherein the bioactive agent is an anti-cancer agent, anti-inflammatory agent, anti-neurodegenerative agent, or anti-immunological agent.

In another aspect, the present disclosure provides a method of treating a disease or disorder in a subject, the method comprising administering to the subject in need thereof a therapeutically effective amount of a bifunctional compound of the present disclosure, or a pharmaceutically acceptable salt, solvate, enantiomer, stereoisomer, or isotopic derivative thereof or a therapeutically effective amount of a pharmaceutical composition of the present disclosure.

### BRIEF DESCRIPTION OF THE DRAWINGS

FIGS. 1A and 1B. Illustration of general principle for PROTAC function. (1A) Exemplary PROTACs comprise a protein targeting moiety (PTM; darkly shaded rectangle), a ubiquitin ligase binding moiety (ULM; lightly shaded triangle), and optionally a linker moiety (L; black line) coupling or tethering the PTM to the ULM. (1B) Illustrates the functional use of the PROTACs as described herein. Briefly, the ULM recognizes and binds to a specific E3 ubiquitin ligase, and the PTM binds and recruits a target protein bringing it into close proximity to the E3 ubiquitin ligase. Typically, the E3 ubiquitin ligase is complexed with an E2 ubiquitin conjugating protein, and either alone or via the E2 protein catalyzes attachment of ubiquitin (dark circles) to a lysine on the target protein via an isopeptide bond. The poly-ubiquitinated protein (far right) is then targeted for degradation by the proteasomal machinery of the cell.

FIG. 2. PD study in C.B.17 SCID mice with OCI-LY1 tumor xenograft. Shows the effect of Example 112 at concentrations of 0 (vehicle only), 5, 15, or 50 mg/kg (mpk) given orally to C.B.17 SCID mice on implanted OCI-Ly1 tumor cells IRAK-4 content.

FIGS. 3A and 3B. PD study in wild type mice. Shows IRAK-4 content in Spleen and lungs of mice that were treated with Example 167 in FIG. 3A, and Example 112 in FIG. 3B at concentrations of 0 (vehicle only), 10, 30 and 90 mg/kg (mpk).

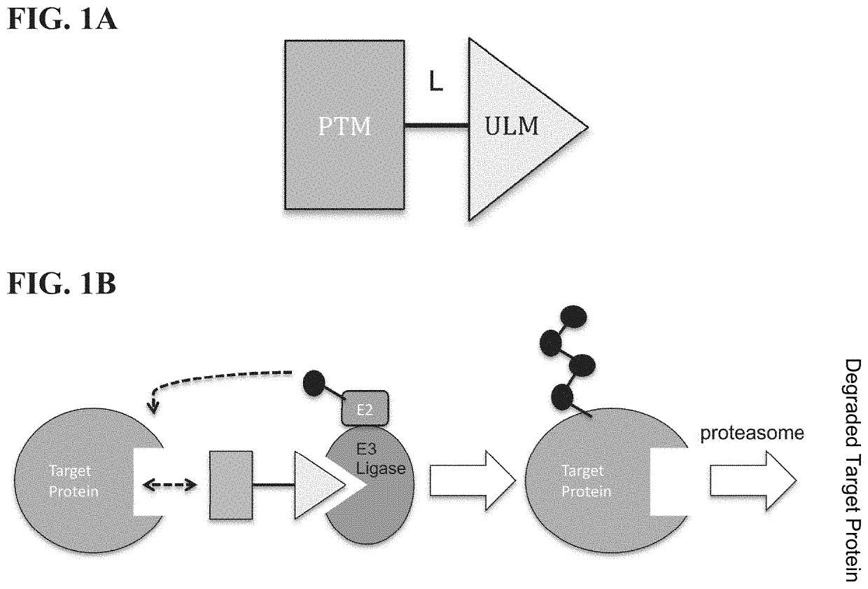

### DETAILED DESCRIPTION

Disclosed herein are bifunctional compounds comprising a target protein binding moiety and a E3 ubiquitin ligase binding moiety, and associated methods of use. The bifunctional compounds are useful as modulators of targeted ubiquitination, especially with respect to Interleukin-1 receptor-associated kinase-4 (IRAK-4), which is degraded and/or otherwise inhibited by bifunctional compounds according to the present disclosure. Specifically, the bifunctional compounds disclosed herein function to recruit endogenous proteins to an E3 ubiquitin ligase for ubiquitination and degradation. The bifunctional or proteolysis targeting chimeric compounds (PROTAC® protein degraders) of the present disclosure find utility as modulators of targeted ubiquitination of, e.g., IRAK-4, which is then degraded and/or otherwise inhibited by the bifunctional compounds described herein.

### Definitions

In the specification, the singular forms (e.g., “a”, “an”, “the”, etc.) also include the plural, unless the context clearly dictates otherwise. Unless defined otherwise, all technical and scientific terms used herein have the same meaning as commonly understood by one of ordinary skill in the art to which this invention belongs. In the case of conflict, the present specification controls. All percentages and ratios used herein, unless otherwise indicated, are by weight.

Throughout the description, where compositions are described as having, including, or comprising specific components, it is contemplated that compositions also consist essentially of, or consist of, the recited components.

The term “alkyl”, as used herein, refers to saturated, straight-chain or branched hydrocarbon radicals containing, in certain embodiments, from one to twenty, including from one to ten, or from one to six, carbon atoms. Branched means that one or more lower C1-C₆ alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl. Examples of C1-C₆ alkyl radicals include, but are not limited to, methyl, ethyl, propyl, isopropyl, butyl, tert-butyl, neopentyl, n-hexyl radicals; and examples of C1-C₈ alkyl radicals include, but are not limited to, methyl, ethyl, propyl, isopropyl, n-butyl, tert-butyl, neopentyl, n-hexyl, heptyl, octyl radicals. Examples of C1-C₂₀ alkyl radicals include but are not limited to hexadecamethyl, hexadecaethyl, hexadecopropyl, octadecamethyl, octadecaethyl, octadecapropyl and the like.

Affixing the suffix “-ene” to a group indicates the group is a divalent moiety, e.g., alkylene (e.g., methylene (—CH₂—), ethylene (—CH₂CH₂—)) is the divalent moiety of alkyl, alkenylene is the divalent moiety of alkenyl, alkynylene is the divalent moiety of alkynyl, heteroalkylene is the divalent moiety of heteroalkyl, cycloalkylene is the divalent moiety of cycloalkyl, heterocycloalkylene is the divalent moiety of heterocycloalkyl, arylene is the divalent moiety of aryl, and heteroarylene is the divalent moiety of heteroaryl.

The term “alkenyl”, as used herein, denotes a monovalent straight or branched group derived from a hydrocarbon moiety containing, in certain embodiments, from two to six, or two to eight, or two to twenty carbon atoms having at least one carbon-carbon double bond. The double bond may or may not be the point of attachment to another group. Examples of C₂-C₈ alkenyl groups include, but are not limited to, for example, ethenyl, propenyl, butenyl, 1-methyl-2-buten-1-yl, heptenyl, octenyl and the like. As defined herein, “akenyl” groups include both cis- and trans-isomers.

The term “alkynyl”, as used herein, denotes a monovalent straight or branched group derived from a hydrocarbon moiety containing, in certain embodiments, from two to six, or two to eight, or two to twenty carbon atoms having at least one carbon-carbon triple bond. The triple bond may or may not be the point of attachment to another group. Examples of C₂-C₈ alkynyl groups include, but are not limited to, for example, ethynyl, propynyl, butynyl and the like.

The term “aromatic” or “aryl”, as used herein, refers to a closed ring structure which has at least one ring having a conjugated pi electron system and includes both carbocyclic aryl and heterocyclic aryl (or “heteroaryl” or “heteroaromatic”) groups. Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C1-C₆) alkyl, (C1-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C1-C₆) alkyl, —C(O)(C1-C₆) alkyl, —OC(O)O(C1-C₆) alkyl, —NH₂, NH((C1-C₆) alkyl), N((C1-C₆) alkyl)₂, —S(O)₂—(C1-C₆) alkyl, —S(O)NH(C1-C₆) alkyl, and —S(O)N((C1-C₆) alkyl)₂. The substituents can themselves be optionally substituted. Furthermore when containing two fused rings, the aryl groups herein defined may have an unsaturated or partially saturated ring fused with a fully saturated ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

The term “C₆-C₁₀ aryl”, as used herein, refers to the cyclic, aromatic hydrocarbon groups phenyl or naphthyl, wherein said C₆-C₁₀ aryl group may be optionally substituted by one or more substituents, e.g., 1 to 5 (for phenyl) or 1 to 7 (for naphthyl) substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C1-C₆) alkyl), N((C₁-C₆) alkyl)₂, —S(O)₂—(C₁-C₆) alkyl, —S(O)NH(C₁-C₆) alkyl, and —S(O)N((C₁-C₆) alkyl)₂. The substituents can themselves be optionally substituted. Furthermore when containing two fused rings the aryl groups herein defined may have an unsaturated or partially saturated ring fused with a fully saturated ring. Exemplary C₆-C₁₀ aryl groups include, but are not limited to, phenyl, naphthyl, and tetrahydronaphthalenyl.

One or more rings may be designated as “aromatic” by a solid circle within the ring(s). This indicates that the bonds and hydrogen atoms of the atoms in the ring are arranged to make the designated ring(s) aromatic. For example, the bicyclic aromatic ring naphthalene may be represented in the following interchangeable ways:

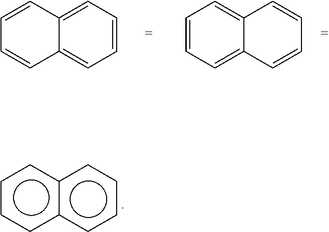

A ring may also be designated as “non-aromatic,” meaning that one of the requirements for aromaticity are not fulfilled. For example, a non-aromatic ring may contain one or more saturated carbons or may be incapable of forming a conjugated pi electron system.

Binders include, but are not limited to, hydroxypropyl methylcellulose (HPMC), hydroxypropyl cellulose (HPC), povidone, copovidone (copolymers of vinylpyrrolidone with other vinyl derivatives), methylcellulose, powdered acacia, gelatin, gum arabicum, guar gum, carbomer such as carbopol, and polymethacrylates.

Carriers include pharmaceutically acceptable excipients and diluents. The term “carrier” means a material, composition, or vehicle, such as a liquid or solid filler, diluent, excipient, solvent, or encapsulating material, involved in carrying or transporting a pharmaceutical agent from one organ, or portion of the body, to another organ, or portion of the body of a subject. Examples include, but are not limited to, calcium carbonate, calcium phosphate, various sugars, starches, cellulose derivatives, gelatin, and polymers such as polyethylene glycols.

The term “cycloalkyl”, as used herein, denotes a monovalent group derived from a monocyclic or polycyclic saturated carbocyclic ring compound. Examples of C₃-C₈-cycloalkyl (3- to 8-membered cycloalkyl) include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo [2.2.1] heptyl, and bicyclo [2.2.2] octyl and the like.

Diluents include, but are not limited to, carbohydrates such as monosaccharides like glucose, oligosaccharides like sucrose and lactose (including anhydrous lactose and lactose monohydrate), starch such as maize starch, potato starch, rice starch and wheat starch, pregelatinized starch, calcium hydrogen phosphate, and sugar alcohols like sorbitol, mannitol, erythritol, and xylitol.

Disintegrants include, but are not limited to, sodium starch glycolate, sodium carboxymethyl cellulose, calcium carboxymethyl cellulose, croscarmellose sodium, crospovidone, chitosan, agar, alginic acid, calcium alginate, methyl cellulose, microcrystalline cellulose, powdered cellulose, lower alkylsubstituted hydroxypropyl cellulose, hydroxylpropyl starch, low-substituted hydroxypropylcellulose, polacrilin potassium, starch, pregelatinized starch, sodium alginate, magnesium aluminum silicate, polacrilin potassium, povidone, sodium starch glycolate, mixtures thereof, and the like.

The term “therapeutically effective amount”, as used herein, refers to an amount of a pharmaceutical agent effective to treat, ameliorate, or prevent an identified disease, condition, or symptom, or to exhibit a detectable therapeutic or inhibitory effect. The effect can be detected by any assay or other detection method known in the art. As used herein, “therapeutically effective amount” can mean that amount necessary to make a clinically observed improvement in the patient. In some embodiments, the composition is formulated such that it comprises an amount that would not cause one or more unwanted side effects. A therapeutically effective amount of a pharmaceutical agent can also mean that amount which provides an objectively identifiable improvement as noted by a clinician or other qualified observer. The precise therapeutically effective amount for a subject will depend upon the subject's age, gender, body weight, size, and health; the nature and extent of the condition; and therapeutic or combination of therapeutics selected for administration. Therapeutically effective amounts for a given situation can be determined by routine experimentation that is within the skill and judgment of the clinician.

Fillers include, but are not limited to, mannitol, sucrose, sorbitol, xylitol, microcrystalline cellulose, lactose, silicic acid, silicified microcrystalline cellulose, hydroxypropyl methylcellulose, hydroxypropyl cellulose, starch, pullulan, and fast dissolving carbohydrates such as Pharmaburst™ fast disintegrating tablets, mixtures thereof, and the like. For examples of fast-dissolving carbohydrates see, e.g., U.S. Pat. No. 8,617,588, which is incorporated herein by reference.

Flavors include, but are not limited to, menthol, peppermint oil, peppermint spirit, vanillin, and almond oil.

Glidants include, but are not limited to, silicon dioxide, colloidal silicon dioxide, calcium silicate, magnesium silicate, magnesium trisilicate, talc, starch, mixtures thereof, and the like.

The terms “haloalkyl”, “haloalkenyl”, or “haloalkynyl”, as used herein refer to an alkyl, alkenyl or alkynyl, including straight-chain and branched, that is substituted with one or more halogens or halo groups. Examples of haloalkyl include but are not limited to CF₃, CH₂CF₃, and CCl₃.

The terms “hal”, “halo”, or “halogen”, as used herein, refer to an atom selected from the group consisting of fluorine, chlorine, bromine, and iodine.

The term “heteroaryl”, as used herein, refers to a mono- or poly-cyclic (e.g., bi-, or tri-cyclic or more) fused or non-fused, radical or ring system having at least one aromatic ring, having from five to twelve ring atoms of which at least one ring atom is selected from the group consisting of S, O, P, and N. In other words, heteroaryl is aryl containing at least one heteroatom. Examples of heteroaryl include, but are not limited to, pyridinyl, furanyl, thiazolyl, imidazolyl, indolyl, benzofuranyl, and the like.

The term “5- or 6-membered heteroaryl”, is taken to mean a ring having five or six ring atoms of which at least one ring atom is selected from the group consisting of S, O, P, and N. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

“Heterocyclyl” or “heterocycloalkyl”, as used herein, are cyclic systems containing carbon and at least one heteroatom selected from N, O, S, and P, wherein there is not delocalized π electrons (aromaticity) shared among the ring carbon or heteroatoms, i.e., the cyclic ring system in non-aromatic. The heterocycloalkyl ring structure may be substituted by one or more substituents. The substituents can themselves be optionally substituted. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “independently selected” is used herein to indicate that, for a variable which occurs in more than one location in a genus, the identity of the variable is determined separately in each instance. For example, if R^{x} appears as a substituent on two different atoms, the two instances of R^{x} may be the same moiety, or different moieties. The same is true if a single atom is substituted with more than one instance of R^{x}. The identity of R^{x} in each instance is determined independently of the identity of the other(s).

“Isomers” mean any compound having an identical molecular formula but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.” A compound that has more than one chiral center has 2n-1 enantiomeric pairs, where n is the number of chiral centers. Compounds with more than one chiral center may exist as either an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). The compounds of Formula (I) may contain asymmetric or chiral centers and, therefore, exist in different stereoisomeric forms. It is intended, unless specified otherwise, that all stereoisomeric forms of the compounds of Formula (I) as well as mixtures thereof, including racemic mixtures, form part of the present invention. In addition, the present invention embraces all geometric and positional isomers (including cis and trans-forms), as well as mixtures thereof, are embraced within the scope of the invention. In general, a reference to a compound is intended to cover its stereoisomers and mixture of various stereoisomers.

The present disclosure is intended to include all isotopes of atoms occurring in the present compounds. Isotopes include those atoms having the same atomic number but different mass numbers. In particular, one, some, or all hydrogens may be deuterium. Radioactive isotopes may be used, for instance for structural analysis or to facilitate tracing the fate of the compounds or their metabolic products after administration. By way of general example and without limitation, isotopes of hydrogen include deuterium and tritium and isotopes of carbon include ¹³C and ¹⁴C.

The term “isotopic derivative” includes derivatives of compounds in which one or more atoms in the compounds are replaced with corresponding isotopes of the atoms. For example, an isotopic derivative of a compound containing a carbon atom (C¹²) would be one in which one or more of the carbon atoms of the compound are replaced with the C¹³ isotope(s).

Lubricants include, but are not limited to, calcium stearate, glyceryl monostearate, glyceryl behenate, glyceryl palmitostearate, hexagonal boron nitride, hydrogenated vegetable oil, light mineral oil, magnesium stearate, mineral oil, polyethylene glycol, poloxamer, sodium benzoate, sodium lauryl sulfate, sodium stearyl fumarate, stearic acid, talc, zinc stearate, mixtures thereof, and the like.

“Oral dosage form” as used herein refers to a pharmaceutical drug product that contains a specified amount (dose) of a compound of the disclosure as the active ingredient, or a pharmaceutically acceptable salt and/or solvate thereof, and inactive components (excipients), formulated into a particular configuration that is suitable for oral administration, such as an oral tablet, liquid, or capsule. In one embodiment, the oral dosage form comprises a tablet. In one embodiment, the oral dosage form comprises a tablet that can be scored. In one embodiment, the oral dosage form comprises a sublingual tablet. In one embodiment, the oral dosage form comprises a capsule, which can be taken intact or used as a sprinkle onto food (e.g., applesauce or yogurt). In one embodiment, the oral dosage form comprises a sachet.

Formulations of the present invention providing “oral administration” as used herein refer to enteral, buccal, sublabial, or sublingual medications in the form of tablets, capsules, syrups, powders, granules, pastilles, solutions, tinctures, elixirs, emulsions, hydrogels, teas, films, disintegrating tablets, mouthwashes, and others.

Suitable forms for oral administration may include one or more pharmaceutically acceptable excipients, including, for example, carriers, fillers, surfactants, diluents, buffers, sweeteners, disintegrants, binders, lubricants, glidants, colorants, flavors, stabilizing agents, coatings, or any mixtures thereof.

A “pharmaceutical composition” is a formulation containing one or more therapeutic agents (e.g., one or more compounds of the present disclosure) in a form suitable for administration to a subject. In one embodiment, the pharmaceutical composition is in bulk form, e.g., for storage. Alternatively, the pharmaceutical composition is in unit dosage form. It can be advantageous to formulate compositions in dosage unit form for ease of administration and uniformity of dosage. Dosage unit form as used herein refers to physically discrete units suited as unitary dosages for the subject to be treated; each unit containing a predetermined quantity of active reagent calculated to produce the desired therapeutic effect in association with the required pharmaceutical carrier. The specifications for the dosage unit forms of the invention are dictated by and directly dependent on the unique characteristics of the active agents and the particular therapeutic effect to be achieved, and the limitations in the art of compounding such an active agent for the treatment of individuals.

A compound of the present disclosure may be administered in the form of a pharmaceutical composition comprising a pharmaceutically acceptable excipient. The formulation may be adapted for administration by any of a variety of routes including oral, buccal, rectal, vaginal, intranasal, intraocular, transdermal, subcutaneous, intravenous, or intramuscular.

The term “pharmaceutical” or “pharmaceutically acceptable” when used herein as an adjective, means substantially non-toxic and substantially non-deleterious to the recipient. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier or excipient” means a carrier or excipient that is useful in preparing a pharmaceutical composition that is generally safe, non-toxic and neither biologically nor otherwise undesirable, and includes any excipient that is acceptable for veterinary use and/or human pharmaceutical use. A “pharmaceutically acceptable excipient” as used herein includes both one and more than one such excipient.

As used herein, “pharmaceutically acceptable salts” can refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts can include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, or an alkaline earth metal ion, e.g., an aluminum ion; or coordinates with an organic base such as ethanolamine, diethanolamine, triethanolamine, tromethamine, N-methylglucamine, diethylamine, diethylaminoethanol, ethylenediamine, imidazole, lysine, arginine, morpholine, 2-hydroxyethylmorpholine, dibenzylethylenediamine, trimethylamine, piperidinyl, pyrrolidine, benzylamine, tetramethylammonium hydroxide and the like.

It should be understood that all references to pharmaceutically acceptable salts include solvent addition forms (solvates) or crystal forms (polymorphs) as defined herein, of the same salt.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

Some of the compounds of the present disclosure may exist in unsolvated as well as solvated forms such as, for example, hydrates.

“Solvate” means a solvent addition form that contains either a stoichiometric or non-stoichiometric amounts of solvent. Some compounds can have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate. In the hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates are sesquihydrates, monohydrates, dihydrates or trihydrates. Also suitable are the hydrates of salts of the compounds of the disclosure.

“Spirocycloalkyl” or “spirocyclyl” means carbogenic bicyclic ring systems with both rings connected through a single atom. The ring can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₃-C₁₂) spirocycloalkyl is a spirocycle containing from 3 to 12 carbon atoms.

It will be appreciated that the compounds, as described herein, may be substituted with one, two, three, four, five or more (up to the total possible number of substitutents for the particular compound) independently selected substituents or functional moieties. In general, the term “substituted” whether preceded by the term “optionally” or not, and substituents contained in formulas disclosed herein, refer to the replacement of hydrogen radicals in a given structure with the radical of a specified substituent. When more than one position in any given structure is substituted with more than one substituent selected from a specified group, the substituent may be either the same or different at each position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this disclosure, heteroatoms such as nitrogen may have hydrogen substituents and/or any permissible substituents of organic compounds described herein which satisfy the valencies of the heteroatoms. The nitrogen and sulfur heteroatoms may optionally be oxidized, and the nitrogen heteroatom may optionally be quaternized. Examples of substituents on the moieties disclosed herein (e.g., alkyl, alkenyl, alkynyl, alkoxy, aryl, heteroaryl, cycloalkyl, cycloalkenyl, non-aromatic heterocycle groups) include, but are not limited to, alkenyl, alkynyl, halogen, haloalkyl, alkoxy, alkylthio, alkylsulfinyl, alkylsulfonyl, heteroaryl, aryl, cycloalkyl, cycloalkenyl, non-aromatic heterocycle, hydroxyl, carbamoyl, oxo, amino, nitro, azido, -SH, and -CN.

As described herein, compounds of the disclosure may optionally be substituted with one or more substituents, such as those described generally above, or as exemplified by particular classes, subclasses, and species of the disclosure. It will be appreciated that the phrase “optionally substituted” is used interchangeably with the phrase “substituted or unsubstituted.” Unless otherwise indicated, an optionally substituted group may have a substituent at any or each substitutable position of the group, and when more than one position in any given structure is substituted with more than one substituent independently selected from a specified group, the substituent may be either the same or different at each substituted every position.

Surfactants include, but are not limited to, non-ionic, anionic, cationic, amphoteric or zwitterionic surfactants. Examples of suitable non-ionic surfactants include ethoxylated triglycerides; fatty alcohol ethoxylates; alkylphenol ethoxylates; fatty acid ethoxylates; fatty amide ethoxylates; fatty amine ethoxylates; sorbitan alkanoates; ethylated sorbitan alkanoates; alkyl ethoxylates; Pluronics™; alkyl polyglucosides; stearol ethoxylates; alkyl polyglycosides. Examples of suitable anionic surfactants include alkylether sulfates; alkylether carboxylates; alkyl benzene sulfonates; alkylether phosphates; dialkyl sulfosuccinates; sarcosinates; alkyl sulfonates; soaps; alkyl sulfates; alkyl carboxylates; alkyl phosphates; paraffin sulfonates; secondary n-alkane sulfonates; alpha-olefin sulfonates; isethionate sulfonates. Examples of suitable cationic surfactants include fatty amine salts; fatty diamine salts; quaternary ammonium compounds; phosphonium surfactants; sulfonium surfactants; sulfoxonium surfactants. Examples of suitable zwitterionic surfactants include N-alkyl derivatives of amino acids (such as glycine, betaine, aminopropionic acid); imidazoline surfactants; amine oxides; amidobetaines. Non-limiting examples of a surfactant that can be used in solid dispersions, include, for example. Tween 20, Tween 80, Span 20, Span 80, sodium docusate (e.g., AOT), sodium lauryl sulfate, and poloxamers (e.g., poloxamer 407, Kolliphor® EL, Pluronic F68). Poloxamers are also known by the trade names Synperonics®, Pluronics®, and Kolliphor®/Cremophor®.

Sweeteners include, but are not limited to, sucrose, high fructose corn syrup, fructose, glucose, aspartame, acesulfame K, sucralose, cyclamate, sodium saccharin, neotame, rebaudioside A, and other stevia-based sweeteners.

Buffers include, but are not limited to, citrate buffer, phosphate buffer, acetate buffer, and bicarbonate buffer.

### Bifunctional Compounds of Formula (Ia) and Formula (I)

In one aspect, this application pertains to a bifunctional compound having the structure of Formula (Ia):

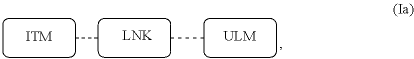

or a pharmaceutically acceptable salt, solvate, enantiomer, stereoisomer, or isotopic derivative thereof, wherein PTM is a protein/polypeptide targeting moiety, LNK is a linker, e.g., a bond (absent) or a chemical group coupling PTM to ULM, and ULM is an E3 ubiquitin ligase binding moiety. The PTM binds to a target protein or polypeptide, which is to be ubiquitinated by a ubiquitin ligase and is chemically linked directly to the ULM group or through a linker moiety LNK.

In one aspect, this application pertains to a bifunctional compound having the structure of Formula (I):

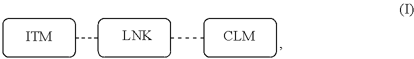

or a pharmaceutically acceptable salt, solvate, enantiomer, stereoisomer, or isotopic derivative thereof wherein:

ITM is a Interleukin-1 Receptor-Associated Kinase 4 (IRAK-4) targeting moiety; LNK is a linker (e.g., a bond or a chemical linker group) covalently coupling the PTM to a cereblon E3 ubiquitin ligase binding moiety or CLM.

In an embodiment, the CLM comprises a chemical group derived from an imide, a thioimide, an amide, or a thioamide. In a particular embodiment, the chemical group is a phthalimido group, or an analog or derivative thereof. In a certain embodiment, the CLM is thalidomide, lenalidomide, pomalidomide, analogs thereof, isosteres thereof, or derivatives thereof. Other contemplated CLMs are described in U.S. Patent Application Publication US2015-0291562, which is incorporated herein in its entirety.

In certain embodiments, “LNK” is a bond. In additional embodiments, the linker “LNK” is a connector with a linear non-hydrogen atom number in the range of 1 to 20. The connector “LNK” can contain, but not limited to the functional groups such as ether, amide, alkane, alkene, alkyne, ketone, hydroxyl, carboxylic acid, thioether, sulfoxide, and sulfone. The linker can contain aromatic, heteroaromatic, cyclic, bicyclic and tricyclic moieties. Substitution with halogen, such as Cl, F, Br, and I can be included in the linker. In the case of fluorine substitution, single or multiple fluorines can be included.

In one aspect, this application pertains to a bifunctional compound having the structure of Formula (I):

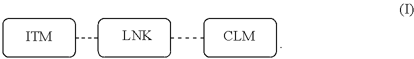

In one aspect, this application pertains to a bifunctional compound having the structure of Formula (I):

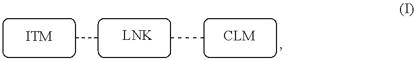

or a pharmaceutically acceptable salt, solvate, enantiomer, stereoisomer, or isotopic derivative thereof,
wherein:
   (a) ITM is an Interleukin-1 Receptor-Associated Kinase 4 (IRAK-4) targeting moiety having the structure of formula ITM-I:

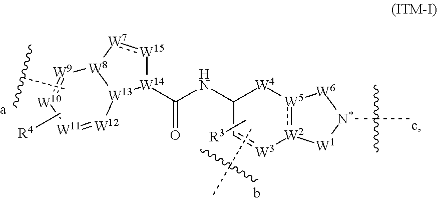

wherein
[symbol] is a single bond or a double bond;
N* is N or NH
W¹ and W⁶ are each independently C═O, CH, O, N, CH₂, CR¹, or NR¹;
W², W³, W⁴, W₅, W⁷, W⁸, W⁹, W¹⁰, W¹¹, W¹², W¹³, W¹⁴, and W¹⁵ are each independently C, N, C(R¹)ₘ, or NR¹;
R¹ is H, —Cl, —F, —Br, —I, N(R²)₂; linear or branched C₁₋₆ alkyl, C₁₋₆ haloalkyl, 3- to 7-membered heterocycloalkyl, or 5- to 6-membered heteroaryl optionally substituted with one, two, three, four, or five R⁶;
R³ and R⁴ are each independently selected from the group consisting of H, —Cl, —F, —Br, —I, linear or branched C₁₋₆ alkyl optionally substituted with one, two, three, four, or five R⁷, OC₁₋₆ alkyl, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C₃₋₇ cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, and C₁₋₆alkyl-C₃₋₇cycloalkyl optionally substituted with one, two, three, four, or five R⁷;
R^{N} is H, linear or branched C₁₋₆ alkyl, or C₁₋₆ haloalkyl;
each R² is independently selected from the group consisting of H and linear or branched C₁₋₆ alkyl;
each R⁶ is independently selected from the group consisting of OH, OC₁₋₆ alkyl, and heterocycloalkyl;
each R⁷ is independently selected from the group consisting of —Cl, —F, —Br, —I, NH₂, CN, CF₃, linear or branched C₁₋₆ alkyl, OC₁₋₆ alkyl, NH(C1-C₆ alkyl), N(C₁₋₆ alkyl)(C₁₋₆ alkyl), C₆-C₁₀ aryl, 5- to 6-membered heteroaryl, C₃-C₇ cycloalkyl, and 3- to 7-membered heterocycloalkyl;
m is 1 or 2; and

ITM is connected to LNK through one of

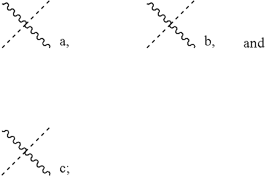

(b) LNK is a chemical linking moiety that covalently couples the ITM to the CLM, having the structure:
[symbol] L [symbol] ₙ
wherein:
each L is independently selected from the group consisting of X, Y, and Z;
each X is independently selected from the group consisting of

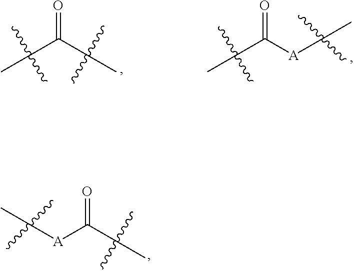

and -A-, wherein each A is independently selected from the group consisting of C(R^{8A})₂, NR⁸, and O;
each R^{8A} is independently selected from the group consisting of H, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, —Cl, —F, —Br, —I, and OH;
each R⁸ is independently selected from the group consisting of H, linear or branched C₁₋₆ alkyl optionally substituted with —Cl, —F, —OH, NH₂, CN, or CF₃, O—C₁₋₆ alkyl, and C₁₋₆ haloalkyl;
each Y is independently selected from the group consisting of

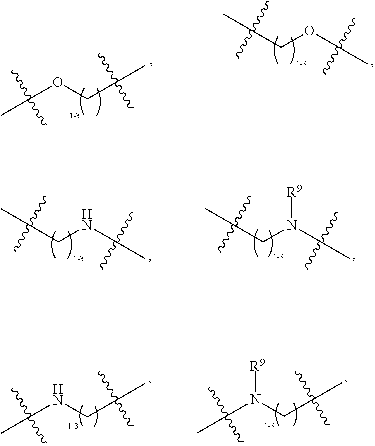

C₁₋₆ alkylene, C₂₋₆ alkenylene, and C₂₋₆ alkynylene;
wherein each Y is independently optionally substituted with one, two, three, four, or five R⁹;
each R⁹ is independently selected from the group consisting of linear or branched C₁₋₆ alkyl optionally substituted with —Cl, —F, —OH, NH₂, CN, or CF₃; O—C₁₋₆ alkyl optionally substituted with —Cl, —F, —OH, NH₂, CN, or CF₃, C₁₋₆ haloalkyl, NH, CN, CF₃, —Cl, —F, —Br, —I, and OH;
each Z is independently selected from the group consisting of monocyclic C₄₋₁₀ cycloalkylene, fused bicyclic C₄₋₁₀ cycloalkylene, bridged bicyclic C₄₋₁₀ cycloalkylene, or spiro-fused bicyclic C₄₋₁₀ cycloalkylene, 4-6 membered heterocycloalkylene, C₆-C₁₀ arylene, and 5-6 membered heteroarylene;
wherein each Z is independently optionally substituted with one, two, three, four, or five R¹⁰;
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH; and
n is 1, 2, 3, 4, 5, or 6;
   (c) CLM is a cereblon E3 ubiquitin ligase binding moiety having a structure selected from the group consisting of formulae CLM-I, CLM-II, and CLM-III:

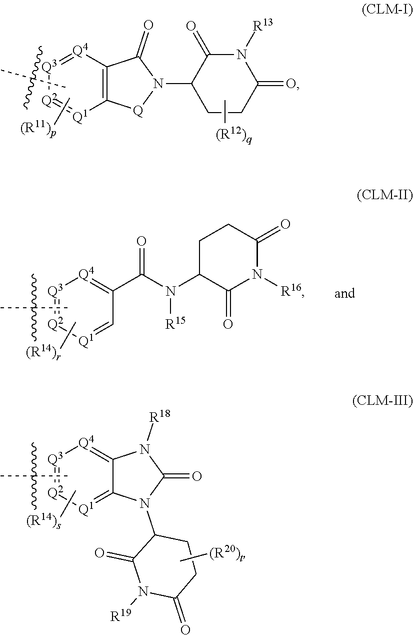

wherein:
Q¹, Q², Q³, and Q⁴ are each independently selected from the group consisting of CH, C, and N;
each R¹¹ is independently selected from the group consisting of —Cl, —F, —Br, —I, CN, CF₃, NH₂, NH—(C1-C₆ alkyl), N(C1-C₆ alkyl)(C1-C₆ alkyl), OH, O—C₁₋₆ alkyl optionally substituted with R²¹, and linear or branched C₁₋₆ alkyl optionally substituted with R²¹;
p is 0, 1, 2, 3, or 4;
Q is selected from the group consisting of CH₂, CH(C₁₋₆ alkyl), C═O, SO₂, NH, and N(C₁₋₆ alkyl);
each R¹² is independently selected from the group consisting of —Cl, —F, —Br, —I, O—C₁₋₆ alkyl optionally substituted with R²¹, NH₂, NH—(C1-C₆ alkyl), N(C1-C₆ alkyl)(C1-C₆ alkyl), OH, CN, CF₃, and linear or branched C₁₋₆ alkyl optionally substituted with R²¹;
q is 0, 1, 2, or 3;
R¹³ is selected from the group consisting of H, OH, linear or branched C₁₋₆ alkyl optionally substituted with R²¹, C(O)C₁₋₆ alkyl, C(O)NH—C₁₋₆ alkyl and C(O)OC₁₋₆ alkyl;
each R¹⁴ is independently selected from the group consisting of —Cl, —F, —Br, —I, NH₂, NH—(C1-C₆ alkyl), N(C1-C₆ alkyl)(C1-C₆ alkyl), OH, CN, CF₃, O—C₁₋₆ alkyl optionally substituted with R²¹, and linear or branched C1-6 alkyl optionally substituted with R²¹;
r is 0, 1, 2, 3, or 4;
R¹⁵ is selected from the group consisting of H and linear or branched C₁₋₆ alkyl optionally substituted with R²¹;
R¹⁶ is selected from the group consisting of H, OH, linear or branched C₁₋₆ alkyl optionally substituted with R²¹, C(O)C₁₋₆ alkyl, C(O)NH—C₁₋₆ alkyl, and C(O)OC₁₋₆ alkyl;
s is 0, 1, 2, or 3;
R¹⁸ is selected from the group consisting of H and linear or branched C₁₋₆ alkyl optionally substituted with R²¹;
R¹⁹ is selected from the group consisting of H, OH, linear or branched C₁₋₆ alkyl optionally substituted with R²¹, C(O)C₁₋₆ alkyl, C(O)NH—C₁₋₆ alkyl, and C(O)OC₁₋₆ alkyl;
each R²⁰ is independently selected from the group consisting of —Cl, —F, —Br, —I, O—C₁₋₆ alkyl optionally substituted with R²¹, NH₂, NH—(C1-C₆ alkyl), N(C1-C₆ alkyl)(C1-C₆ alkyl), OH, CN, CF₃, and linear or branched C₁₋₆ alkyl optionally substituted with R²¹;
t is 0, 1, 2, or 3;
each R²¹ is independently selected from the group consisting of —Cl, —F, —OH, NH₂, CN, and CF₃; and
[symbol] is the point of attachment to LNK.

In one embodiment, the ITM has the structure of formula ITM-I:

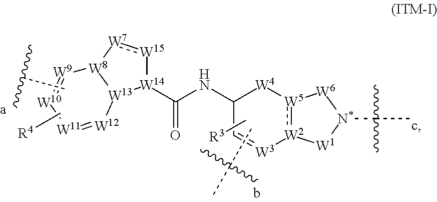

wherein [symbol]
is a single bond or a double bond;

### N* is N or NH

W¹ and W⁶ are each independently C═O, CH, O, N, CH₂, CR¹, or NR¹;
W², W³, W⁴, W₅, W⁷, W⁸, W⁹, W¹⁰, W¹¹, W¹², W¹³, W¹⁴, and W¹⁵ are each independently C, N, C(R¹)ₘ, or NR¹;
R¹ is H, —Cl, —F, —Br, —I, N(R²)₂; linear or branched C₁₋₆ alkyl, C₁₋₆ haloalkyl, 3- to 7-membered heterocycloalkyl, or 5- to 6-membered heteroaryl optionally substituted with one, two, three, four, or five R⁶;
R³ and R⁴ are each independently selected from the group consisting of H, —Cl, —F, —Br, —I, linear or branched C₁₋₆ alkyl optionally substituted with one, two, three, four, or five R⁷, OC₁₋₆ alkyl, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C₃₋₇ cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, and C₁₋₆alkyl-C₃₋₇cycloalkyl optionally substituted with one, two, three, four, or five R⁷;
R^{N} is H, linear or branched C₁₋₆ alkyl, or C₁₋₆ haloalkyl;
each R² is independently selected from the group consisting of H and linear or branched C₁₋₆ alkyl;
each R⁶ is independently selected from the group consisting of OH, OC₁₋₆ alkyl, and heterocycloalkyl;
each R⁷ is independently selected from the group consisting of —Cl, —F, —Br, —I, NH₂, CN, CF₃, linear or branched C₁₋₆ alkyl, OC₁₋₆ alkyl, NH(C1-C₆ alkyl), N(C₁₋₆ alkyl)(C₁₋₆ alkyl), C₆-C₁₀ aryl, 5- to 6-membered heteroaryl, C₃-C₇ cycloalkyl, and 3- to 7-membered heterocycloalkyl;
m is 1 or 2; and
ITM is connected to LNK through one of

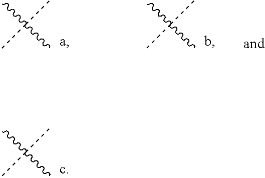

In one embodiment, the ITM has the structure of formula ITM-Ia:

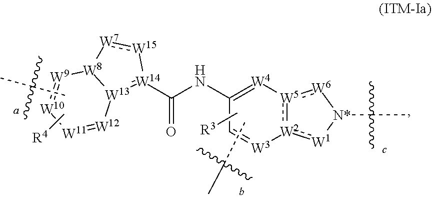

wherein [symbol]
is a single bond or a double bond;

### N* is N or NH

W¹ and W⁶ are each independently C═O, CH, O, N, CH₂, CR¹, or NR¹;
W², W³, W⁴, W⁵, W⁷, W⁸, W⁹, W¹⁰, W¹¹, W¹², W¹³, W¹⁴, and W¹⁵ are each independently C, N, C(R¹)ₘ, or NR¹;
R¹ is H, —Cl, —F, —Br, —I, N(R²)₂; linear or branched C₁₋₆ alkyl, C₁₋₆ haloalkyl, 3- to 7-membered heterocycloalkyl, or 5- to 6-membered heteroaryl optionally substituted with one, two, three, four, or five R⁶;
R³ and R⁴ are each independently selected from the group consisting of H, —Cl, —F, —Br, —I, linear or branched C₁₋₆ alkyl optionally substituted with one, two, three, four, or five R⁷, OC₁₋₆ alkyl optionally substituted with one, two, three, four, or five R⁷, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C₃₋₇ cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, OC₃₋₇ heterocycloalkyl optionally substituted with one, two, three, four, or five R⁷ and C₁₋₆alkyl-C₃₋₇cycloalkyl optionally substituted with one, two, three, four, or five R⁷;
R^{N} is H, linear or branched C₁₋₆ alkyl, or C₁₋₆ haloalkyl;
each R² is independently selected from the group consisting of H and linear or branched C₁₋₆ alkyl; each R⁶ is independently selected from the group consisting of OH, OC₁₋₆ alkyl, and heterocycloalkyl;
each R⁷ is independently selected from the group consisting of —Cl, —F, —Br, —I, NH₂, CN, CF₃, linear or branched C₁₋₆ alkyl, linear or branched C₁₋₆ haloalkyl, OC₁₋₆ alkyl, NH(C1-C₆ alkyl), N(C₁₋₆ alkyl)(C₁₋₆ alkyl), C₆-C₁₀ aryl, 5- to 6-membered heteroaryl, C₃-C₇ cycloalkyl optionally substituted with one, two, three, four, or five R^{7a}, and 3- to 7-membered heterocycloalkyl;
each R^{7a} is independently selected from the group consisting of —Cl, —F, —Br, —I, NH₂, CN, and CF₃;
m is 1 or 2; and
ITM is connected to LNK through one of a

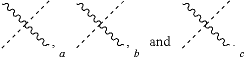

In one embodiment, ITM is connected to LNK through

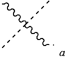

In one embodiment, ITM is connected to LNK through

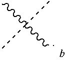

In one embodiment, ITM is connected to LNK through

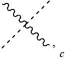

In one embodiment, W¹ is C═O. In one embodiment, W¹ is CH. In one embodiment, W¹ is N. In one embodiment, W¹ is CH₂. In one embodiment, W¹ is CR¹. In one embodiment, W¹ is NR¹.

In one embodiment, W² is C. In one embodiment, W² is N. In one embodiment, W² is CR¹. In one embodiment, W² is NR¹.

In one embodiment, W³ is C. In one embodiment, W³ is N. In one embodiment, W³ is CR¹. In one embodiment, W³ is NR¹.

In one embodiment, W⁴ is C. In one embodiment, W⁴ is N. In one embodiment, W⁴ is CR¹. In one embodiment, W⁴ is NR¹.

In one embodiment, W⁵ is C. In one embodiment, W⁵ is N. In one embodiment, W⁵ is CR¹. In one embodiment, W⁵ is NR¹.

In one embodiment, W⁶ is C═O. In one embodiment, W⁶ is CH. In one embodiment, W⁶ is N. In one embodiment, W⁶ is O. In one embodiment, W⁶ is CH₂. In one embodiment, W⁶ is CR¹. In one embodiment, W⁶ is NR¹.

In one embodiment, W⁷ is C. In one embodiment, W⁷ is N. In one embodiment, W⁷ is C(R¹)ₘ. In one embodiment, W⁷ is NR¹.

In one embodiment, W⁸ is C. In one embodiment, W⁸ is N. In one embodiment, W⁸ is CR¹. In one embodiment, W⁸ is NR¹.

In one embodiment, W⁹ is C. In one embodiment, W⁹ is N. In one embodiment, W⁹ is CR¹. In one embodiment, W⁹ is NR¹.

In one embodiment, W¹⁰ is C. In one embodiment, W¹⁰ is N. In one embodiment, W¹⁰ is CR¹. In one embodiment, W¹⁰ is NR¹.

In one embodiment, W¹¹ is C. In one embodiment, W¹¹ is N. In one embodiment, W¹¹ is CR¹. In one embodiment, W¹¹ is NR¹.

In one embodiment, W¹² is C. In one embodiment, W¹² is N. In one embodiment, W¹² is CR¹. In one embodiment, W¹² is NR¹.

In one embodiment, W¹³ is C. In one embodiment, W¹³ is N. In one embodiment, W¹³ is CR¹. In one embodiment, W¹³ is NR¹.

In one embodiment, W¹⁴ is C. In one embodiment, W¹⁴ is N. In one embodiment, W¹⁴ is CR¹. In one embodiment, W¹⁴ is NR¹.

In one embodiment, W¹⁵ is C. In one embodiment, W¹⁵ is N. In one embodiment, W¹⁵ is C(R¹)ₘ. In one embodiment, W¹⁵ is NR¹.

In one embodiment, at least one R¹ is H. In one embodiment, at least one R¹ is —Cl. In one embodiment, at least one R¹ is —F. In one embodiment, at least one R¹ is —Br. In one embodiment, at least one R¹ is —I. In one embodiment, at least one R¹ is N(R²)₂.

In one embodiment, R² is H. In one embodiment, R² is linear or branched C₁₋₆ alkyl.

In one embodiment, R¹ is linear or branched C₁₋₆ alkyl. In one embodiment, R¹ is C₁₋₆ haloalkyl. In one embodiment, R¹ is heterocycloalkyl. In one embodiment, R¹ is heteroaryl optionally substituted with one, two, three, four, or five R⁶.

In one embodiment, R^{N} is H. In one embodiment, R^{N} is linear or branched C₁₋₆ alkyl. In one embodiment, R^{N} is methyl. In one embodiment, R^{N} is ethyl.

In one embodiment, R^{N} is C₁₋₆ haloalkyl.

In one embodiment, R³ is H. In one embodiment, R³ is —Cl. In one embodiment, R³ is —F. In one embodiment, R³ is —Br. In one embodiment, R³ is —I. In one embodiment, R³ is linear or branched C₁₋₆ alkyl. In one embodiment, R³ is linear or branched C₁₋₆ alkyl substituted with one, two, three, four, or five R⁷. In one embodiment, R³ is OC₁₋₆ alkyl. In one embodiment, R³ is OC₁ alkyl. In one embodiment, R³ is OC₂ alkyl. In one embodiment, R³ is OC₃ alkyl. In one embodiment, R³ is OC₄ alkyl. In one embodiment, R³ is OC₅ alkyl. In one embodiment, R³ is OC₆ alkyl. In one embodiment, R³ is OC₁₋₆ alkyl-C₃₋₇ cycloalkyl.

In one embodiment, R³ is monocyclic C₃₋₇ cycloalkyl, fused bicyclic C₃₋₇ cycloalkyl, bridged bicyclic C₃₋₇ cycloalkyl, or spiro-fused bicyclic C₃₋₇ cycloalkyl.

In one embodiment, R³ is monocyclic C₃₋₇ cycloalkyl substituted with one, two, three, four, or five R⁷, fused bicyclic C₃₋₇ cycloalkyl substituted with one, two, three, four, or five R⁷, bridged bicyclic C₃₋₇ cycloalkyl substituted with one, two, three, four, or five R⁷, or spiro-fused bicyclic C₃₋₇ cycloalkyl substituted with one, two, three, four, or five R⁷.

In one embodiment, R³ is monocyclic heterocycloalkyl, fused bicyclic heterocycloalkyl, bridged bicyclic heterocycloalkyl, or spiro-fused bicyclic heterocycloalkyl.

In one embodiment, R³ is monocyclic heterocycloalkyl substituted with one, two, three, four, or five R⁷, fused bicyclic heterocycloalkyl substituted with one, two, three, four, or five R⁷, bridged bicyclic heterocycloalkyl substituted with one, two, three, four, or five R⁷, or spiro-fused bicyclic heterocycloalkyl substituted with one, two, three, four, or five R⁷.

In one embodiment, R³ is azetidinyl. In one embodiment, R³ is pyrrolidinyl. In one embodiment, R³ is piperidinyl. In one embodiment, R³ is morpholinyl. In one embodiment, R³ is piperazinyl. In one embodiment R³ is selected from the group consisting of oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

In one embodiment, R³ is azetidinyl substituted with one, two, three, four, or five R⁷. In one embodiment, R³ is pyrrolidinyl substituted with one, two, three, four, or five R⁷. In one embodiment, R³ is piperidinyl substituted with one, two, three, four, or five R⁷. In one embodiment, R³ is morpholinyl substituted with one, two, three, four, or five R⁷. In one embodiment, R³ is piperazinyl substituted with one, two, three, four, or five R⁷.

In one embodiment, R³ is OC₃₋₇ cycloalkyl. In one embodiment, R³ is OC₃ cycloalkyl. In one embodiment, R³ is OC₄ cycloalkyl. In one embodiment, R³ is OC₅ cycloalkyl. In one embodiment, R³ is OC₆ cycloalkyl. In one embodiment, R³ is OC₇ cycloalkyl.

In one embodiment, R³ is OC₃₋₇ cycloalkyl substituted with one, two, three, four, or five R⁷. In one embodiment, R³ is OC₃ cycloalkyl substituted with one, two, three, four, or five R⁷. In one embodiment, R³ is OC₄ cycloalkyl substituted with one, two, three, four, or five R⁷. In one embodiment, R³ is OC₅ cycloalkyl substituted with one, two, three, four, or five R⁷. In one embodiment, R³ is OC₆ cycloalkyl substituted with one, two, three, four, or five R⁷. In one embodiment, R³ is OC₇ cycloalkyl substituted with one, two, three, four, or five R⁷.

In one embodiment, R³ is C₁₋₆alkyl-C₃₋₇cycloalkyl. In one embodiment, R³ is C₁₋₆alkyl-C₃₋₇cycloalkyl substituted with one, two, three, four, or five R⁷.

In one embodiment, R³ is selected from the group consisting of:

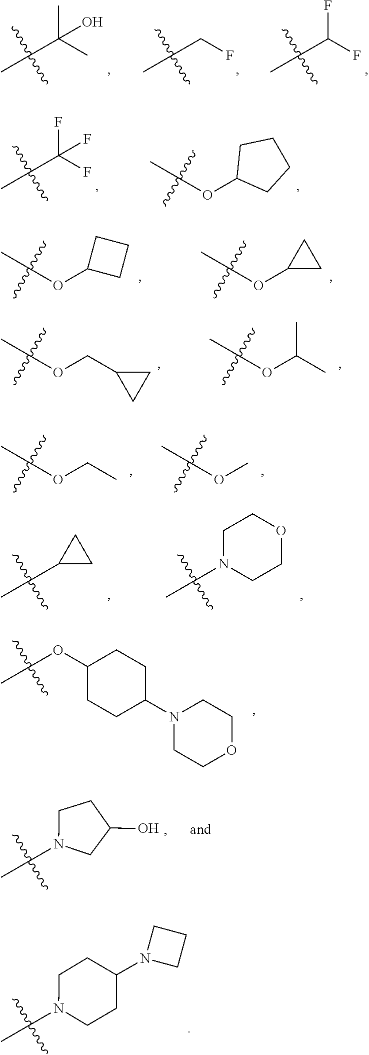

In one embodiment, R⁴ is H. In one embodiment, R⁴ is —Cl. In one embodiment, R⁴ is —F. In one embodiment, R⁴ is —Br. In one embodiment, R⁴ is —I.

In one embodiment, R⁴ is linear or branched C₁₋₆ alkyl. In one embodiment, R⁴ is linear or branched C₁₋₆ alkyl substituted with one, two, three, four, or five R⁷.

In one embodiment, R⁴ is OC₁₋₆ alkyl. In one embodiment, R⁴ is OC₁ alkyl. In one embodiment, R⁴ is OC₂ alkyl. In one embodiment, R⁴ is OC₃ alkyl. In one embodiment, R⁴ is OC₄ alkyl. In one embodiment, R⁴ is OC₅ alkyl. In one embodiment, R⁴ is OC₆ alkyl.

In one embodiment, R⁴ is OC₁₋₆ alkyl-C₃₋₇ cycloalkyl.

In one embodiment, R⁴ is monocyclic C₃₋₇ cycloalkyl, fused bicyclic C₃₋₇ cycloalkyl, bridged bicyclic C₃₋₇ cycloalkyl, or spiro-fused bicyclic C₃₋₇ cycloalkyl. In one embodiment, R⁴ is monocyclic C₃₋₇ cycloalkyl, fused bicyclic C₃₋₇ cycloalkyl, bridged bicyclic C₃₋₇ cycloalkyl, or spiro-fused bicyclic C₃₋₇ cycloalkyl, each substituted with one, two, three, four, or five R⁷.

In one embodiment, R⁴ is monocyclic, fused bicyclic heterocycloalkyl, bridged bicyclic heterocycloalkyl, or spiro-fused bicyclic heterocycloalkyl. In one embodiment, R⁴ is monocyclic heterocycloalkyl, fused bicyclic heterocycloalkyl, bridged bicyclic heterocycloalkyl, or spiro-fused bicyclic heterocycloalkyl, each substituted with one, two, three, four, or five R⁷.

In one embodiment, R⁴ is azetidinyl. In one embodiment, R⁴ is pyrrolidinyl. In one embodiment, R⁴ is piperidinyl. In one embodiment, R⁴ is morpholinyl. In one embodiment, R⁴ is piperazinyl.

In one embodiment, R⁴ is azetidinyl substituted with one, two, three, four, or five R⁷. In one embodiment, R⁴ is pyrrolidinyl substituted with one, two, three, four, or five R⁷. In one embodiment, R⁴ is piperidinyl substituted with one, two, three, four, or five R⁷. In one embodiment, R⁴ is morpholinyl substituted with one, two, three, four, or five R⁷. In one embodiment, R⁴ is piperazinyl substituted with one, two, three, four, or five R⁷.

In one embodiment, R⁴ is OC₃₋₇ cycloalkyl. In one embodiment, R⁴ is OC₃ cycloalkyl. In one embodiment, R⁴ is OC₄ cycloalkyl. In one embodiment, R⁴ is OC₅ cycloalkyl. In one embodiment, R⁴ is OC₆ cycloalkyl. In one embodiment, R⁴ is OC₇ cycloalkyl. In one embodiment, R⁴ is OC₃₋₇ cycloalkyl substituted with one, two, three, four, or five R⁷. In one embodiment, R⁴ is OC₃ cycloalkyl substituted with one, two, three, four, or five R⁷. In one embodiment, R⁴ is OC₄ cycloalkyl substituted with one, two, three, four, or five R⁷. In one embodiment, R⁴ is OCs cycloalkyl substituted with one, two, three, four, or five R⁷. In one embodiment, R⁴ is OC₆ cycloalkyl substituted with one, two, three, four, or five R⁷. In one embodiment, R⁴ is OC₇ cycloalkyl substituted with one, two, three, four, or five R⁷.

In one embodiment, R⁴ is C₁₋₆alkyl-C₃₋₇cycloalkyl. In one embodiment, R⁴ is C₁₋₆alkyl-C₃₋₇cycloalkyl substituted with one, two, three, four, or five R⁷.

In one embodiment, each R⁶ is independently selected from the group consisting of OC₁₋₆ alkyl and heterocycloalkyl. In one embodiment, each R⁶ is independently selected from the group consisting of OH and heterocycloalkyl. In one embodiment, each R⁶ is independently selected from the group consisting of OH and OC₁₋₆ alkyl.

In one embodiment, each R⁶ is OH. In one embodiment, each R⁶ is OC₁₋₆ alkyl. In one embodiment, each R⁶ is heterocycloalkyl.

In one embodiment, each R⁷ is independently selected from the group consisting of —Cl, —F, —Br, —I, NH₂, CN, CF₃, linear or branched C₁₋₆ alkyl, and OC₁₋₆ alkyl. In one embodiment, R⁷ is —Cl. In one embodiment, R⁷ is —F. In one embodiment, R⁷ is —Br. In one embodiment, R⁷ is —I. In one embodiment, R⁷ is NH₂. In one embodiment, R⁷ is CN. In one embodiment, R⁷ is CF₃. In one embodiment, R⁷ is linear or branched C₁₋₆ alkyl. In one embodiment, R⁷ is OC₁₋₆ alkyl.

In one embodiment, each R⁷ is independently selected from the group consisting of NH(C1-C₆ alkyl) and N(C₁₋₆ alkyl)(C₁₋₆ alkyl). In one embodiment, R⁷ is NH(C1-C₆ alkyl).

In one embodiment, R⁷ is N(C₁₋₆ alkyl)(C₁₋₆ alkyl).

In one embodiment, each R⁷ is independently selected from the group consisting of C₆-C₁₀ aryl, 5- to 6-membered heteroaryl, C₃-C₇ cycloalkyl, and 3- to 7-membered heterocycloalkyl. In one embodiment, R⁷ is C₆-C₁₀ aryl. In one embodiment, R⁷ is C₅-C₆ heteroaryl. In one embodiment, R⁷ is C₃-C₇ cycloalkyl. In one embodiment, R⁷ is 3- to 7-membered heterocycloalkyl.

In one embodiment, m is 1. In one embodiment, m is 2.

In one embodiment, the ITM is selected from the group consisting of:

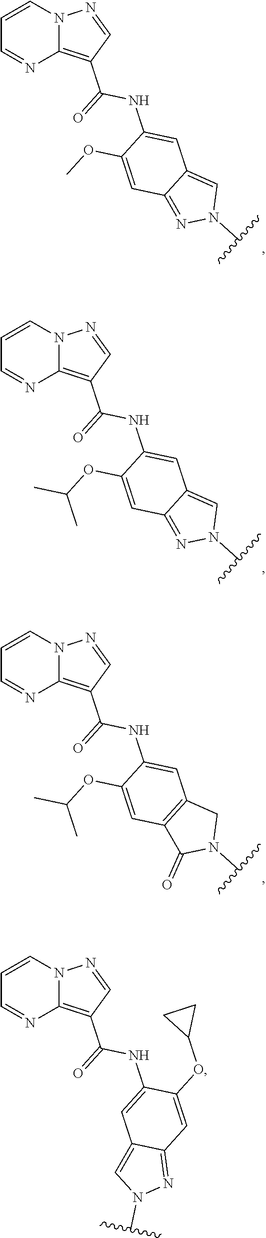 [SMILES: *n1cc2cc(NC(=O)c3cnn4cccnc34)c(OC)cc2n1 ; *n1cc2cc(NC(=O)c3cnn4cccnc34)c(OC(C)C)cc2n1 ; *N1Cc2cc(NC(=O)c3cnn4cccnc34)c(OC(C)C)cc2C1=O ; *n1cc2cc(NC(=O)c3cnn4cccnc34)c(OC3CC3)cc2n1]

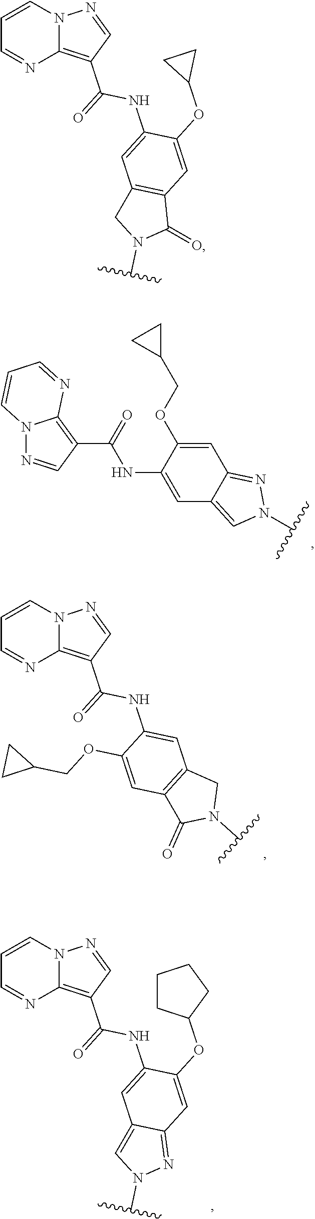 [SMILES: *N1Cc2cc(NC(=O)c3cnn4cccnc34)c(OC3CC3)cc2C1=O ; *n1cc2cc(NC(=O)c3cnn4cccnc34)c(OCC3CC3)cc2n1 ; *N1Cc2cc(NC(=O)c3cnn4cccnc34)c(OCC3CC3)cc2C1=O ; *n1cc2cc(NC(=O)c3cnn4cccnc34)c(OC3CCCC3)cc2n1]

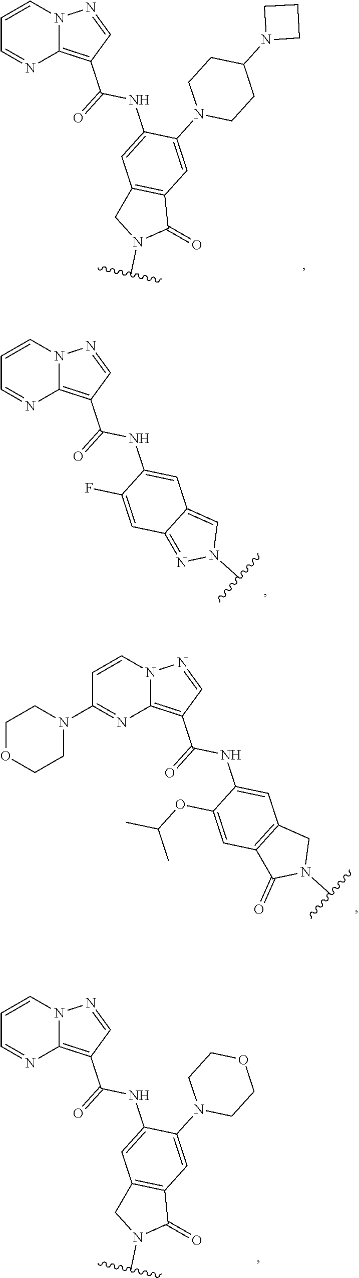 [SMILES: *N1Cc2cc(NC(=O)c3cnn4cccnc34)c(N3CCC(N4CCC4)CC3)cc2C1=O ; *n1cc2cc(NC(=O)c3cnn4cccnc34)c(F)cc2n1 ; *N1Cc2cc(NC(=O)c3cnn4ccc(N5CCOCC5)nc34)c(OC(C)C)cc2C1=O ; *N1Cc2cc(NC(=O)c3cnn4cccnc34)c(N3CCOCC3)cc2C1=O]

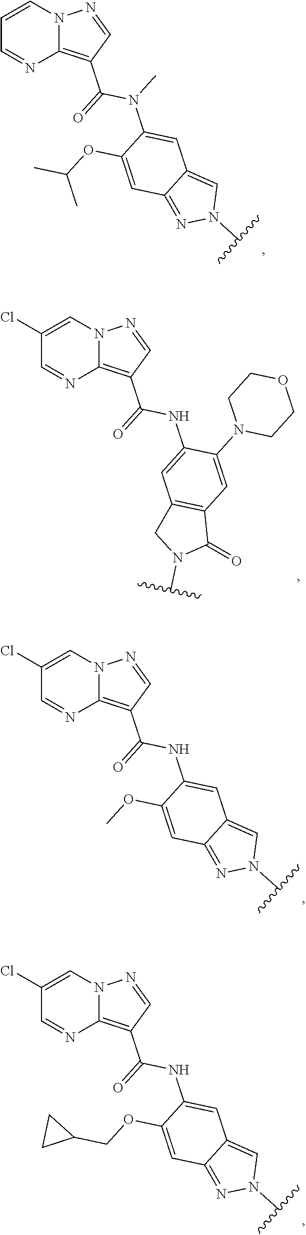 [SMILES: *n1cc2cc(N(C)C(=O)c3cnn4cccnc34)c(OC(C)C)cc2n1 ; *N1Cc2cc(NC(=O)c3cnn4cc(Cl)cnc34)c(N3CCOCC3)cc2C1=O ; *n1cc2cc(NC(=O)c3cnn4cc(Cl)cnc34)c(OC)cc2n1 ; *n1cc2cc(NC(=O)c3cnn4cc(Cl)cnc34)c(OCC3CC3)cc2n1]

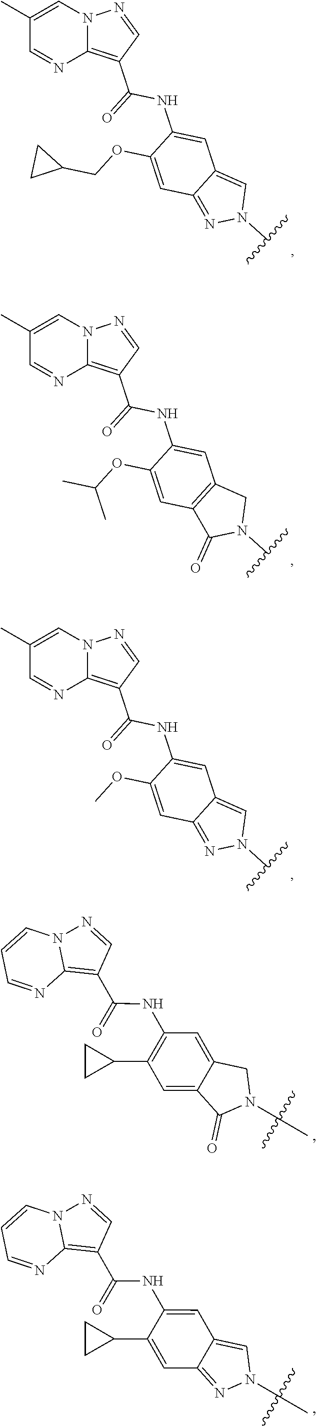 [SMILES: *n1cc2cc(NC(=O)c3cnn4cc(C)cnc34)c(OCC3CC3)cc2n1 ; *N1Cc2cc(NC(=O)c3cnn4cc(C)cnc34)c(OC(C)C)cc2C1=O ; *n1cc2cc(NC(=O)c3cnn4cc(C)cnc34)c(OC)cc2n1 ; *N1Cc2cc(NC(=O)c3cnn4cccnc34)c(C3CC3)cc2C1=O ; *n1cc2cc(NC(=O)c3cnn4cccnc34)c(C3CC3)cc2n1]

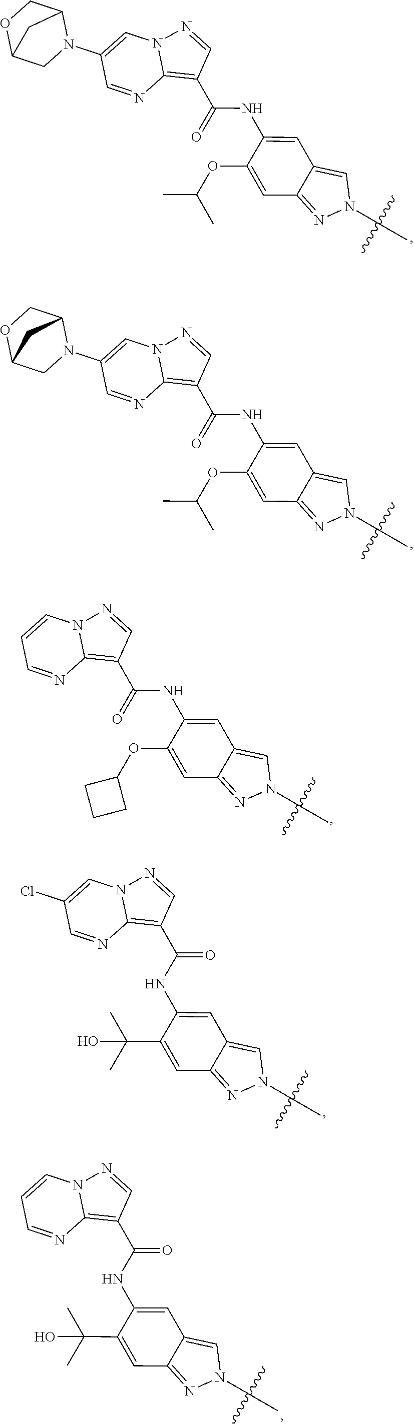 [SMILES: *n1cc2cc(NC(=O)c3cnn4cc(N5CC6CC5CO6)cnc34)c(OC(C)C)cc2n1 ; *n1cc2cc(NC(=O)c3cnn4cc(N5C[C@H]6C[C@@H]5CO6)cnc34)c(OC(C)C)cc2n1 ; *n1cc2cc(NC(=O)c3cnn4cccnc34)c(OC3CCC3)cc2n1 ; *n1cc2cc(NC(=O)c3cnn4cc(Cl)cnc34)c(C(C)(C)O)cc2n1 ; *n1cc2cc(NC(=O)c3cnn4cccnc34)c(C(C)(C)O)cc2n1]

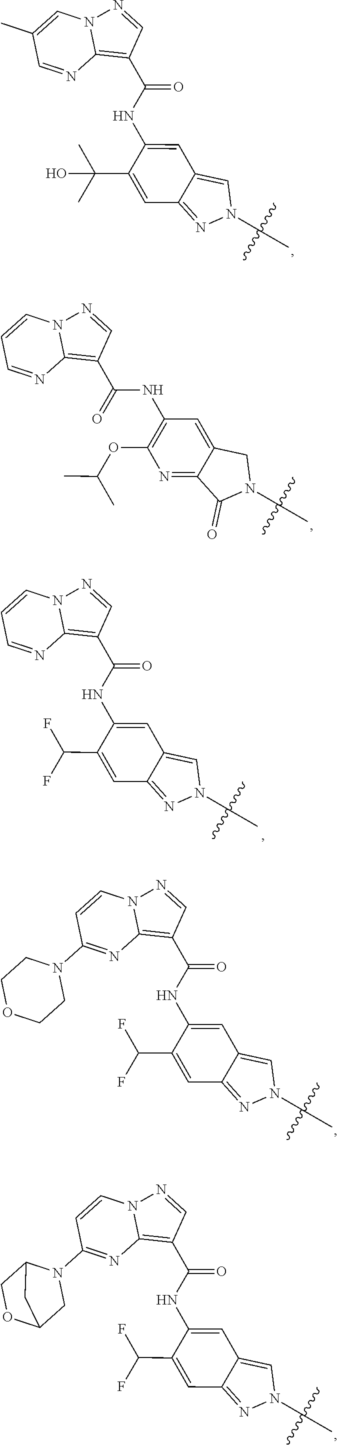 [SMILES: *n1cc2cc(NC(=O)c3cnn4cc(C)cnc34)c(C(C)(C)O)cc2n1 ; *N1Cc2cc(NC(=O)c3cnn4cccnc34)c(OC(C)C)nc2C1=O ; *n1cc2cc(NC(=O)c3cnn4cccnc34)c(C(F)F)cc2n1 ; *n1cc2cc(NC(=O)c3cnn4ccc(N5CCOCC5)nc34)c(C(F)F)cc2n1 ; *n1cc2cc(NC(=O)c3cnn4ccc(N5CC6CC5CO6)nc34)c(C(F)F)cc2n1]

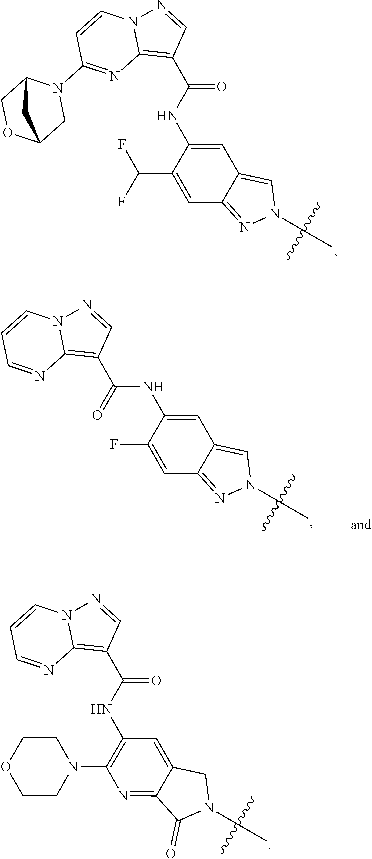 [SMILES: *n1cc2cc(NC(=O)c3cnn4ccc(N5C[C@H]6C[C@@H]5CO6)nc34)c(C(F)F)cc2n1 ; *n1cc2cc(NC(=O)c3cnn4cccnc34)c(F)cc2n1 ; *N1Cc2cc(NC(=O)c3cnn4cccnc34)c(N3CCOCC3)nc2C1=O]

In one embodiment, the LNK is a chemical linking moiety that covalently couples the ITM to the CLM.

In one embodiment, the LNK is:
[symbol] L [symbol] ₙ, wherein each L is independently selected from the group consisting of X, Y, and Z; and
n is 1, 2, 3, 4, 5, or 6.

In one embodiment, n is 1.

In one embodiment, the linker is —X—. In one embodiment, the linker is —Y—. In one embodiment, the linker is —Z—.

In one embodiment, n is 2.

In one embodiment, the linker is —X—X—. In one embodiment, the linker is —Y—X—. In one embodiment, the linker is —Z—X—. In one embodiment, the linker is —X—Y—. In one embodiment, the linker is —Y—Y—. In one embodiment, the linker is —Z—Y—. In one embodiment, the linker is —X—Z—. In one embodiment, the linker is —Y—Z—. In one embodiment, the linker is —Z—Z—.

In one embodiment, n is 3.

In one embodiment, the linker is —X—X—X—. In one embodiment, the linker is —X—X—Y—. In one embodiment, the linker is —X—X—Z—. In one embodiment, the linker is —X—Y—X—. In one embodiment, the linker is —X—Y—Y—. In one embodiment, the linker is —X—Y—Z—. In one embodiment, the linker is —X—Z—X—. In one embodiment, the linker is —X—Z—Y—. In one embodiment, the linker is —X—Z—Z—.

In one embodiment, the linker is —Y—X—X—. In one embodiment, the linker is —Y—X—Y—. In one embodiment, the linker is —Y—X—Z—. In one embodiment, the linker is —Y—Y—X—. In one embodiment, the linker is —Y—Y—Y—. In one embodiment, the linker is —Y—Y—Z—. In one embodiment, the linker is —Y—Z—X—. In one embodiment, the linker is —Y—Z—Y—. In one embodiment, the linker is —Y—Z—Z—.

In one embodiment, the linker is —Z—X—X—. In one embodiment, the linker is —Z—X—Y—. In one embodiment, the linker is —Z—X—Z—. In one embodiment, the linker is —Z—Y—X—. In one embodiment, the linker is —Z—Y—Y—. In one embodiment, the linker is —Z—Y—Z—. In one embodiment, the linker is —Z—Z—X—. In one embodiment, the linker is —Z—Z—Y—. In one embodiment, the linker is —Z—Z—Z—.

In one embodiment, n is 4. In one embodiment, the linker is —X—X—X—X—. In one embodiment, the linker is —X—X—X—Y—. In one embodiment, the linker is —X—X—X—Z—. In one embodiment, the linker is —X—X—Y—X—. In one embodiment, the linker is —X—X—Y—Y—. In one embodiment, the linker is —X—X—Y—Z—. In one embodiment, the linker is —X—X—Z—X—. In one embodiment, the linker is —X—X—Z—Y—. In one embodiment, the linker is —X—X—Z—Z—. In one embodiment, the linker is —X—Y—X—X—. In one embodiment, the linker is —X—Y—X—Y—. In one embodiment, the linker is —X—Y—X—Z—. In one embodiment, the linker is —X—Y—Y—X—. In one embodiment, the linker is —X—Y—Y—Y—. In one embodiment, the linker is —X—Y—Y—Z—. In one embodiment, the linker is —X—Y—Z—X—. In one embodiment, the linker is —X—Y—Z—Y—. In one embodiment, the linker is —X—Y—Z—Z—. In one embodiment, the linker is —X—Z—X—X—. In one embodiment, the linker is —X—Z—X—Y—. In one embodiment, the linker is —X—Z—X—Z—. In one embodiment, the linker is —X—Z—Y—X—. In one embodiment, the linker is —X—Z—Y—Y—. In one embodiment, the linker is —X—Z—Y—Z—. In one embodiment, the linker is —X—Z—Z—X—. In one embodiment, the linker is —X—Z—Z—Y—. In one embodiment, the linker is —X—Z—Z—Z—. In one embodiment, the linker is —Y—X—X—X—. In one embodiment, the linker is —Y—X—X—Y—. In one embodiment, the linker is —Y—X—X—Z—. In one embodiment, the linker is —Y—X—Y—X—. In one embodiment, the linker is —Y—X—Y—Y—. In one embodiment, the linker is —Y—X—Y—Z—. In one embodiment, the linker is —Y—X—Z—X—. In one embodiment, the linker is —Y—X—Z—Y—. In one embodiment, the linker is —Y—X—Z—Z—. In one embodiment, the linker is —Y—Y—X—X—. In one embodiment, the linker is —Y—Y—X—Y—. In one embodiment, the linker is —Y—Y—X—Z—. In one embodiment, the linker is —Y—Y—Y—X—. In one embodiment, the linker is —Y—Y—Y—Y—. In one embodiment, the linker is —Y—Y—Y—Z—. In one embodiment, the linker is —Y—Y—Z—X—. In one embodiment, the linker is —Y—Y—Z—Y—. In one embodiment, the linker is —Y—Y—Z—Z—. In one embodiment, the linker is —Y—Z—X—X—. In one embodiment, the linker is —Y—Z—X—Y—. In one embodiment, the linker is —Y—Z—X—Z—. In one embodiment, the linker is —Y—Z—Y—X—. In one embodiment, the linker is —Y—Z—Y—Y—. In one embodiment, the linker is —Y—Z—Y—Z—. In one embodiment, the linker is —Y—Z—Z—X—. In one embodiment, the linker is —Y—Z—Z—Y—. In one embodiment, the linker is —Y—Z—Z—Z—. In one embodiment, the linker is —Z—X—X—X—. In one embodiment, the linker is —Z—X—X—Y—. In one embodiment, the linker is —Z—X—X—Z—. In one embodiment, the linker is —Z—X—Y—X—. In one embodiment, the linker is —Z—X—Y—Y—. In one embodiment, the linker is —Z—X—Y—Z—. In one embodiment, the linker is —Z—X—Z—X—. In one embodiment, the linker is —Z—X—Z—Y—. In one embodiment, the linker is —Z—X—Z—Z—. In one embodiment, the linker is —Z—Y—X—X—. In one embodiment, the linker is —Z—Y—X—Y—. In one embodiment, the linker is —Z—Y—X—Z—. In one embodiment, the linker is —Z—Y—Y—X—. In one embodiment, the linker is —Z—Y—Y—Y—. In one embodiment, the linker is —Z—Y—Y—Z—. In one embodiment, the linker is —Z—Y—Z—X—. In one embodiment, the linker is —Z—Y—Z—Y—. In one embodiment, the linker is —Z—Y—Z—Z—. In one embodiment, the linker is —Z—Z—X—X—. In one embodiment, the linker is —Z—Z—X—Y—. In one embodiment, the linker is —Z—Z—X—Z—. In one embodiment, the linker is —Z—Z—Y—X—. In one embodiment, the linker is —Z—Z—Y—Y—. In one embodiment, the linker is —Z—Z—Y—Z—. In one embodiment, the linker is —Z—Z—Z—X—. In one embodiment, the linker is —Z—Z—Z—Y—. In one embodiment, the linker is —Z—Z—Z—Z—.

In one embodiment, n is 5. In one embodiment, the linker is —X—X—X—X—X—. In one embodiment, the linker is —X—X—X—X—Y—. In one embodiment, the linker is —X—X—X—X—Z—. In one embodiment, the linker is —X—X—X—Y—X—. In one embodiment, the linker is —X—X—X—Y—Y—. In one embodiment, the linker is —X—X—X—Y—Z—. In one embodiment, the linker is —X—X—X—Z—X—. In one embodiment, the linker is —X—X—X—Z—Y—. In one embodiment, the linker is —X—X—X—Z—Z—. In one embodiment, the linker is —X—X—Y—X—X—. In one embodiment, the linker is —X—X—Y—X—Y—. In one embodiment, the linker is —X—X—Y—X—Z—. In one embodiment, the linker is —X—X—Y—Y—X—. In one embodiment, the linker is —X—X—Y—Y—Y—. In one embodiment, the linker is —X—X—Y—Y—Z—. In one embodiment, the linker is —X—X—Y—Z—X—. In one embodiment, the linker is —X—X—Y—Z—Y—. In one embodiment, the linker is —X—X—Y—Z—Z—. In one embodiment, the linker is —X—X—Z—X—X—. In one embodiment, the linker is —X—X—Z—X—Y—. In one embodiment, the linker is —X—X—Z—X—Z—. In one embodiment, the linker is —X—X—Z—Y—X—. In one embodiment, the linker is —X—X—Z—Y—Y—. In one embodiment, the linker is —X—X—Z—Y—Z—. In one embodiment, the linker is —X—X—Z—Z—X—. In one embodiment, the linker is —X—X—Z—Z—Y—. In one embodiment, the linker is —X—X—Z—Z—Z—. In one embodiment, the linker is —X—Y—X—X—X—. In one embodiment, the linker is —X—Y—X—X—Y—. In one embodiment, the linker is —X—Y—X—X—Z—. In one embodiment, the linker is —X—Y—X—Y—X—. In one embodiment, the linker is —X—Y—X—Y—Y—. In one embodiment, the linker is —X—Y—X—Y—Z—. In one embodiment, the linker is —X—Y—X—Z—X—. In one embodiment, the linker is —X—Y—X—Z—Y—. In one embodiment, the linker is —X—Y—X—Z—Z—. In one embodiment, the linker is —X—Y—Y—X—X—. In one embodiment, the linker is —X—Y—Y—X—Y—. In one embodiment, the linker is —X—Y—Y—X—Z—. In one embodiment, the linker is —X—Y—Y—Y—X—. In one embodiment, the linker is —X—Y—Y—Y—Y—. In one embodiment, the linker is —X—Y—Y—Y—Z—. In one embodiment, the linker is —X—Y—Y—Z—X—. In one embodiment, the linker is —X—Y—Y—Z—Y—. In one embodiment, the linker is —X—Y—Y—Z—Z—. In one embodiment, the linker is —X—Y—Z—X—X—. In one embodiment, the linker is —X—Y—Z—X—Y—. In one embodiment, the linker is —X—Y—Z—X—Z—. In one embodiment, the linker is —X—Y—Z—Y—X—. In one embodiment, the linker is —X—Y—Z—Y—Y—. In one embodiment, the linker is —X—Y—Z—Y—Z—. In one embodiment, the linker is —X—Y—Z—Z—X—. In one embodiment, the linker is —X—Y—Z—Z—Y—. In one embodiment, the linker is —X—Y—Z—Z—Z—. In one embodiment, the linker is —X—Z—X—X—X—. In one embodiment, the linker is —X—Z—X—X—Y—. In one embodiment, the linker is —X—Z—X—X—Z—. In one embodiment, the linker is —X—Z—X—Y—X—. In one embodiment, the linker is —X—Z—X—Y—Y—. In one embodiment, the linker is —X—Z—X—Y—Z—. In one embodiment, the linker is —X—Z—X—Z—X—. In one embodiment, the linker is —X—Z—X—Z—Y—. In one embodiment, the linker is —X—Z—X—Z—Z—. In one embodiment, the linker is —X—Z—Y—X—X—. In one embodiment, the linker is —X—Z—Y—X—Y—. In one embodiment, the linker is —X—Z—Y—X—Z—. In one embodiment, the linker is —X—Z—Y—Y—X—. In one embodiment, the linker is —X—Z—Y—Y—Y—. In one embodiment, the linker is —X—Z—Y—Y—Z—. In one embodiment, the linker is —X—Z—Y—Z—X—. In one embodiment, the linker is —X—Z—Y—Z—Y—. In one embodiment, the linker is —X—Z—Y—Z—Z—. In one embodiment, the linker is —X—Z—Z—X—X—. In one embodiment, the linker is —X—Z—Z—X—Y—. In one embodiment, the linker is —X—Z—Z—X—Z—. In one embodiment, the linker is —X—Z—Z—Y—X—. In one embodiment, the linker is —X—Z—Z—Y—Y—. In one embodiment, the linker is —X—Z—Z—Y—Z—. In one embodiment, the linker is —X—Z—Z—Z—X—. In one embodiment, the linker is —X—Z—Z—Z—Y—. In one embodiment, the linker is —X—Z—Z—Z—Z—. In one embodiment, the linker is —Y—X—X—X—X—. In one embodiment, the linker is —Y—X—X—X—Y—. In one embodiment, the linker is —Y—X—X—X—Z—. In one embodiment, the linker is —Y—X—X—Y—X—. In one embodiment, the linker is —Y—X—X—Y—Y—. In one embodiment, the linker is —Y—X—X—Y—Z—. In one embodiment, the linker is —Y—X—X—Z—X—. In one embodiment, the linker is —Y—X—X—Z—Y—. In one embodiment, the linker is —Y—X—X—Z—Z—. In one embodiment, the linker is —Y—X—Y—X—X—. In one embodiment, the linker is —Y—X—Y—X—Y—. In one embodiment, the linker is —Y—X—Y—X—Z—. In one embodiment, the linker is —Y—X—Y—Y—X—. In one embodiment, the linker is —Y—X—Y—Y—Y—. In one embodiment, the linker is —Y—X—Y—Y—Z—. In one embodiment, the linker is —Y—X—Y—Z—X—. In one embodiment, the linker is —Y—X—Y—Z—Y—. In one embodiment, the linker is —Y—X—Y—Z—Z—. In one embodiment, the linker is —Y—X—Z—X—X—. In one embodiment, the linker is —Y—X—Z—X—Y—. In one embodiment, the linker is —Y—X—Z—X—Z—. In one embodiment, the linker is —Y—X—Z—Y—X—. In one embodiment, the linker is —Y—X—Z—Y—Y—. In one embodiment, the linker is —Y—X—Z—Y—Z—. In one embodiment, the linker is —Y—X—Z—Z—X—. In one embodiment, the linker is —Y—X—Z—Z—Y—. In one embodiment, the linker is —Y—X—Z—Z—Z—. In one embodiment, the linker is —Y—Y—X—X—X—. In one embodiment, the linker is —Y—Y—X—X—Y—. In one embodiment, the linker is —Y—Y—X—X—Z—. In one embodiment, the linker is —Y—Y—X—Y—X—. In one embodiment, the linker is —Y—Y—X—Y—Y—. In one embodiment, the linker is —Y—Y—X—Y—Z—. In one embodiment, the linker is —Y—Y—X—Z—X—. In one embodiment, the linker is —Y—Y—X—Z—Y—. In one embodiment, the linker is —Y—Y—X—Z—Z—. In one embodiment, the linker is —Y—Y—Y—X—X—. In one embodiment, the linker is —Y—Y—Y—X—Y—. In one embodiment, the linker is —Y—Y—Y—X—Z—. In one embodiment, the linker is —Y—Y—Y—Y—X—. In one embodiment, the linker is —Y—Y—Y—Y—Y—. In one embodiment, the linker is —Y—Y—Y—Y—Z—. In one embodiment, the linker is —Y—Y—Y—Z—X—. In one embodiment, the linker is —Y—Y—Y—Z—Y—. In one embodiment, the linker is —Y—Y—Y—Z—Z—. In one embodiment, the linker is —Y—Y—Z—X—X—. In one embodiment, the linker is —Y—Y—Z—X—Y—. In one embodiment, the linker is —Y—Y—Z—X—Z—. In one embodiment, the linker is —Y—Y—Z—Y—X—. In one embodiment, the linker is —Y—Y—Z—Y—Y—. In one embodiment, the linker is —Y—Y—Z—Y—Z—. In one embodiment, the linker is —Y—Y—Z—Z—X—. In one embodiment, the linker is —Y—Y—Z—Z—Y—. In one embodiment, the linker is —Y—Y—Z—Z—Z—. In one embodiment, the linker is —Y—Z—X—X—X—. In one embodiment, the linker is —Y—Z—X—X—Y—. In one embodiment, the linker is —Y—Z—X—X—Z—. In one embodiment, the linker is —Y—Z—X—Y—X—. In one embodiment, the linker is —Y—Z—X—Y—Y—. In one embodiment, the linker is —Y—Z—X—Y—Z—. In one embodiment, the linker is —Y—Z—X—Z—X—. In one embodiment, the linker is —Y—Z—X—Z—Y—. In one embodiment, the linker is —Y—Z—X—Z—Z—. In one embodiment, the linker is —Y—Z—Y—X—X—. In one embodiment, the linker is —Y—Z—Y—X—Y—. In one embodiment, the linker is —Y—Z—Y—X—Z—. In one embodiment, the linker is —Y—Z—Y—Y—X—. In one embodiment, the linker is —Y—Z—Y—Y—Y—. In one embodiment, the linker is —Y—Z—Y—Y—Z—. In one embodiment, the linker is —Y—Z—Y—Z—X—. In one embodiment, the linker is —Y—Z—Y—Z—Y—. In one embodiment, the linker is —Y—Z—Y—Z—Z—. In one embodiment, the linker is —Y—Z—Z—X—X—. In one embodiment, the linker is —Y—Z—Z—X—Y—. In one embodiment, the linker is —Y—Z—Z—X—Z—. In one embodiment, the linker is —Y—Z—Z—Y—X—. In one embodiment, the linker is —Y—Z—Z—Y—Y—. In one embodiment, the linker is —Y—Z—Z—Y—Z—. In one embodiment, the linker is —Y—Z—Z—Z—X—. In one embodiment, the linker is —Y—Z—Z—Z—Y—. In one embodiment, the linker is —Y—Z—Z—Z—Z—. In one embodiment, the linker is —Z—X—X—X—X—. In one embodiment, the linker is —Z—X—X—X—Y—. In one embodiment, the linker is —Z—X—X—X—Z—. In one embodiment, the linker is —Z—X—X—Y—X—. In one embodiment, the linker is —Z—X—X—Y—Y—. In one embodiment, the linker is —Z—X—X—Y—Z—. In one embodiment, the linker is —Z—X—X—Z—X—. In one embodiment, the linker is —Z—X—X—Z—Y—. In one embodiment, the linker is —Z—X—X—Z—Z—. In one embodiment, the linker is —Z—X—Y—X—X—. In one embodiment, the linker is —Z—X—Y—X—Y—. In one embodiment, the linker is —Z—X—Y—X—Z—. In one embodiment, the linker is —Z—X—Y—Y—X—. In one embodiment, the linker is —Z—X—Y—Y—Y—. In one embodiment, the linker is —Z—X—Y—Y—Z—. In one embodiment, the linker is —Z—X—Y—Z—X—. In one embodiment, the linker is —Z—X—Y—Z—Y—. In one embodiment, the linker is —Z—X—Y—Z—Z—. In one embodiment, the linker is —Z—X—Z—X—X—. In one embodiment, the linker is —Z—X—Z—X—Y—. In one embodiment, the linker is —Z—X—Z—X—Z—. In one embodiment, the linker is —Z—X—Z—Y—X—. In one embodiment, the linker is —Z—X—Z—Y—Y—. In one embodiment, the linker is —Z—X—Z—Y—Z—. In one embodiment, the linker is —Z—X—Z—Z—X—. In one embodiment, the linker is —Z—X—Z—Z—Y—. In one embodiment, the linker is —Z—X—Z—Z—Z—. In one embodiment, the linker is —Z—Y—X—X—X—. In one embodiment, the linker is —Z—Y—X—X—Y—. In one embodiment, the linker is —Z—Y—X—X—Z—. In one embodiment, the linker is —Z—Y—X—Y—X—. In one embodiment, the linker is —Z—Y—X—Y—Y—. In one embodiment, the linker is —Z—Y—X—Y—Z—. In one embodiment, the linker is —Z—Y—X—Z—X—. In one embodiment, the linker is —Z—Y—X—Z—Y—. In one embodiment, the linker is —Z—Y—X—Z—Z—. In one embodiment, the linker is —Z—Y—Y—X—X—. In one embodiment, the linker is —Z—Y—Y—X—Y—. In one embodiment, the linker is —Z—Y—Y—X—Z—. In one embodiment, the linker is —Z—Y—Y—Y—X—. In one embodiment, the linker is —Z—Y—Y—Y—Y—. In one embodiment, the linker is —Z—Y—Y—Y—Z—. In one embodiment, the linker is —Z—Y—Y—Z—X—. In one embodiment, the linker is —Z—Y—Y—Z—Y—. In one embodiment, the linker is —Z—Y—Y—Z—Z—. In one embodiment, the linker is —Z—Y—Z—X—X—. In one embodiment, the linker is —Z—Y—Z—X—Y—. In one embodiment, the linker is —Z—Y—Z—X—Z—. In one embodiment, the linker is —Z—Y—Z—Y—X—. In one embodiment, the linker is —Z—Y—Z—Y—Y—. In one embodiment, the linker is —Z—Y—Z—Y—Z—. In one embodiment, the linker is —Z—Y—Z—Z—X—. In one embodiment, the linker is —Z—Y—Z—Z—Y—. In one embodiment, the linker is —Z—Y—Z—Z—Z—. In one embodiment, the linker is —Z—Z—X—X—X—. In one embodiment, the linker is —Z—Z—X—X—Y—. In one embodiment, the linker is —Z—Z—X—X—Z—. In one embodiment, the linker is —Z—Z—X—Y—X—. In one embodiment, the linker is —Z—Z—X—Y—Y—. In one embodiment, the linker is —Z—Z—X—Y—Z—. In one embodiment, the linker is —Z—Z—X—Z—X—. In one embodiment, the linker is —Z—Z—X—Z—Y—. In one embodiment, the linker is —Z—Z—X—Z—Z—. In one embodiment, the linker is —Z—Z—Y—X—X—. In one embodiment, the linker is —Z—Z—Y—X—Y—. In one embodiment, the linker is —Z—Z—Y—X—Z—. In one embodiment, the linker is —Z—Z—Y—Y—X—. In one embodiment, the linker is —Z—Z—Y—Y—Y—. In one embodiment, the linker is —Z—Z—Y—Y—Z—. In one embodiment, the linker is —Z—Z—Y—Z—X—. In one embodiment, the linker is —Z—Z—Y—Z—Y—. In one embodiment, the linker is —Z—Z—Y—Z—Z—. In one embodiment, the linker is —Z—Z—Z—X—X—. In one embodiment, the linker is —Z—Z—Z—X—Y—. In one embodiment, the linker is —Z—Z—Z—X—Z—. In one embodiment, the linker is —Z—Z—Z—Y—X—. In one embodiment, the linker is —Z—Z—Z—Y—Y—. In one embodiment, the linker is —Z—Z—Z—Y—Z—. In one embodiment, the linker is —Z—Z—Z—Z—X—. In one embodiment, the linker is —Z—Z—Z—Z—Y—. In one embodiment, the linker is —Z—Z—Z—Z—Z—.

In one embodiment, n is 6. In one embodiment, the linker is —Y—Y—Y—Y—Y—Z—. In one embodiment, the linker is —X—Y—Y—Y—Y—Z—. In one embodiment, the linker is —X—Z—Y—Y—Y—Y—. In one embodiment, the linker is —Z—Y—Y—Y—Y—Y—.

In one embodiment, each X is independently selected from the group consisting of,

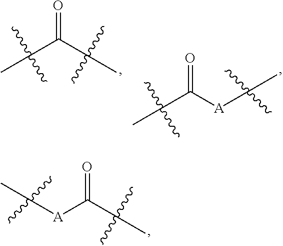

and -A-, wherein each A is independently selected from the group consisting of C(R^{8A})₂, NR⁸, and O.

In one embodiment, each R^{8A} is independently selected from the group consisting of H, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, —Cl, —F, —Br, —I, and OH.

In one embodiment, each R^{8A} is H. In one embodiment, each R^{8A} is linear or branched C₁₋₆ alkyl. In one embodiment, one R^{8A} is H and the other R^{8A} is linear C₁₋₆ alkyl. In one embodiment, one R^{8A} is H and the other R^{8A} is branched C₁₋₆ alkyl.

In one embodiment, each R^{8A} is independently selected from the group consisting of H and linear or branched C₁₋₆ alkyl optionally substituted with —Cl, —F, —OH, NH₂, CN, or CF₃. In one embodiment, each R^{8A} is independently selected from the group consisting of H and O—C₁₋₆ alkyl optionally substituted with —Cl, —F, —OH, NH₂, CN, or CF₃. In one embodiment, each R^{8A} is independently selected from the group consisting of H and C₁₋₆ haloalkyl.

In one embodiment, each R^{8A} is independently selected from the group consisting of H, NH₂, CN, CF₃, —Cl, —F, —Br, —I, and OH.

In one embodiment, each R⁸ is independently selected from the group consisting of H, linear or branched C₁₋₆ alkyl optionally substituted with —Cl, —F, —OH, NH₂, CN, or CF₃, O—C₁₋₆ alkyl, and C₁₋₆ haloalkyl. In one embodiment, each R⁸ is independently selected from the group consisting of H and linear or branched C₁₋₆ alkyl optionally substituted with —Cl, —F, —OH, NH₂, CN, or CF₃. In one embodiment, each R⁸ is independently selected from the group consisting of H and O—C₁₋₆ alkyl. In one embodiment, each R⁸ is independently selected from the group consisting of H and C₁₋₆ haloalkyl.

In one embodiment, each Y is independently selected from the group consisting of

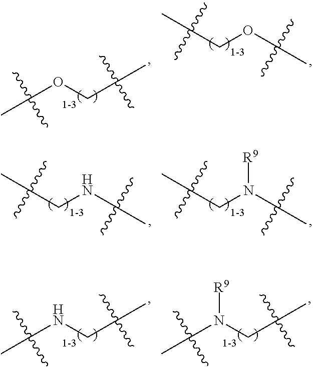

C₁₋₆ alkylene, C₂₋₆ alkenylene, and C₂₋₆ alkynylene.

In one embodiment, Y is

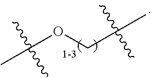

In one embodiment, Y is

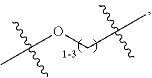

substituted with one, two, three, four, or five R⁹.

In one embodiment, Y is

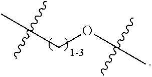

In one embodiment, Y is

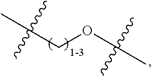

substituted with one, two, three, four, or five R⁹.

In one embodiment, Y is

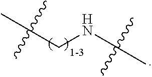

In one embodiment, Y is

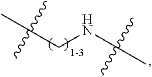

substituted with one, two, three, four, or five R⁹.

In one embodiment, Y is

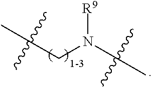

In one embodiment, Y is

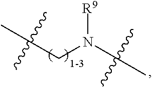

substituted with one, two, three, four, or five R⁹.

In one embodiment, Y is

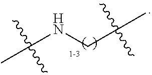

In one embodiment, Y is

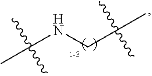

substituted with one, two, three, four, or five R⁹.

In one embodiment, Y is

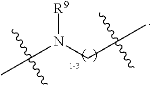

In one embodiment, Y is

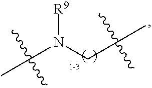

substituted with one, two, three, four, or five R⁹.

In one embodiment, Y is C₁₋₆ alkylene.

In one embodiment, Y is C₁₋₆ alkylene, substituted with one, two, three, four, or five R⁹.

In one embodiment, Y is C₂₋₆ alkenylene.

In one embodiment, Y is C₂₋₆ alkenylene, substituted with one, two, three, four, or five R⁹.

In one embodiment, Y is C₂₋₆ alkynylene.

In one embodiment, Y is C₂₋₆ alkynylene, substituted with one, two, three, four, or five R⁹.

In one embodiment, each R⁹ is independently selected from the group consisting of linear or branched C₁₋₆ alkyl optionally substituted with —Cl, —F, —OH, NH₂, CN, or CF₃ and O—C₁₋₆ alkyl optionally substituted with —Cl, —F, —OH, NH₂, CN, or CF₃.

In one embodiment, each R⁹ is independently selected from the group consisting of linear or branched C₁₋₆ alkyl optionally substituted with —Cl, —F, —OH, NH₂, CN, or CF₃ and C₁₋₆ haloalkyl.

In one embodiment, each R⁹ is independently selected from the group consisting of NH, CN, CF₃, —Cl, —F, —Br, —I, OH, and linear or branched C₁₋₆ alkyl optionally substituted with —Cl, —F, —OH, NH₂, CN, or CF₃.

In one embodiment, each R⁹ is linear or branched C₁₋₆ alkyl.

In one embodiment, each R⁹ is linear or branched C₁₋₆ alkyl substituted with —Cl, —F, —OH, NH₂, CN, or CF₃.

In one embodiment, Z is monocyclic C₄₋₁₀ cycloalkylene. In one embodiment, Z is monocyclic C₄₋₁₀ cycloalkylene substituted with one, two, three, four, or five R¹⁰.

In one embodiment, Z is bridged bicyclic C₄₋₁₀ cycloalkylene. In one embodiment, Z is bridged bicyclic C₄₋₁₀ cycloalkylene substituted with one, two, three, four, or five R¹⁰.

In one embodiment, Z is spiro-fused bicyclic C₄₋₁₀ cycloalkylene. In one embodiment, Z is spiro-fused bicyclic C₄₋₁₀ cycloalkylene substituted with one, two, three, four, or five R¹⁰.

In one embodiment, Z is 4-6 membered heterocycloalkylene. In one embodiment, Z is 4-6 membered heterocycloalkylene substituted with one, two, three, four, or five R¹⁰.

In one embodiment, Z is C₆-C₁₀ arylene. In one embodiment, Z is C₆-C₁₀ arylene substituted with one, two, three, four, or five R¹⁰.

In one embodiment, Z is 5-6 membered heteroarylene. In one embodiment, Z is 5-6 membered heteroarylene substituted with one, two, three, four, or five R¹⁰.

In one embodiment, R¹⁰ is linear or branched C₁₋₆ alkyl. In one embodiment, R¹⁰ is linear or branched C₁₋₆ alkyl, optionally substituted with —Cl, —F, —OH, NH₂, CN, or CF₃.

In one embodiment, R¹⁰ is —Cl, —F, —Br, or —I.

In one embodiment, R¹⁰ is O—C₁₋₆ alkyl. In one embodiment, R¹⁰ is O—C₁₋₆ alkyl optionally substituted with —Cl, —F, —OH, NH₂, CN, or CF₃.

In one embodiment, R¹⁰ is C₁₋₆ haloalkyl.

In one embodiment, R¹⁰ is NH₂, CN, CF₃, or OH. In one embodiment, R¹⁰ is NH₂. In one embodiment, R¹⁰ is CN. In one embodiment, R¹⁰ is CF₃. In one embodiment, R¹⁰ is OH.

In one embodiment, the LNK is:

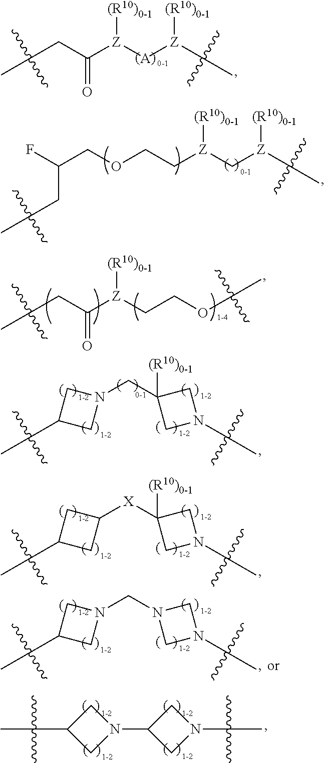

wherein each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH;
each A is independently selected from the group consisting of CH₂, NH, and O;
X is selected from the group consisting of CH₂, NH, and O; and
Z is selected from the group consisting of CH₂, CH, C, NH, N, and O.

In one embodiment, the LNK is:

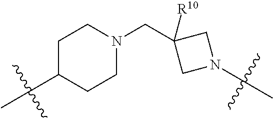

wherein R¹⁰ is H. In one embodiment, the LNK is:

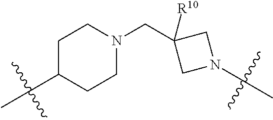

wherein R¹⁰ is —F.

In one embodiment, the LNK is:

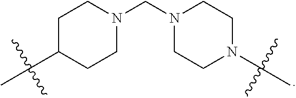 [SMILES: *C1CCN(CN2CCN(*)CC2)CC1]

In one embodiment, the LNK is:

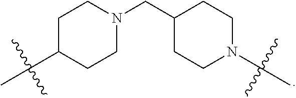 [SMILES: *C1CCN(CC2CCN(*)CC2)CC1]

In one embodiment, the LNK is:

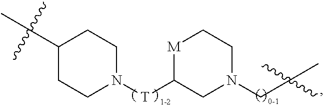

wherein M is CH₂; and each T is independently selected from the group consisting of CH₂, —CH₂—O—, —CH₂—O—CH₂—, —CH₂CH₂—O—, —CH₂CH₂—O—CH₂CH₂—, —CH₂—O—CH₂CH₂—, —CH₂CH₂—O—CH₂—, —C(═O)—O—, and —C(═O)—NH—.

In one embodiment, the LNK is:

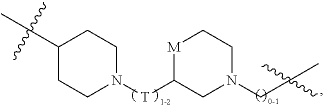

wherein M is O; and each T is independently selected from the group consisting of CH₂, —CH₂—O—, —CH₂—O—CH₂—, —CH₂CH₂—O—, —CH₂CH₂—O—CH₂CH₂—, —CH₂—O—CH₂CH₂—, —CH₂CH₂—O—CH₂—, —C(═O)—O—, and —C(═O)—NH—.

In one embodiment, the LNK is selected from the group consisting of:

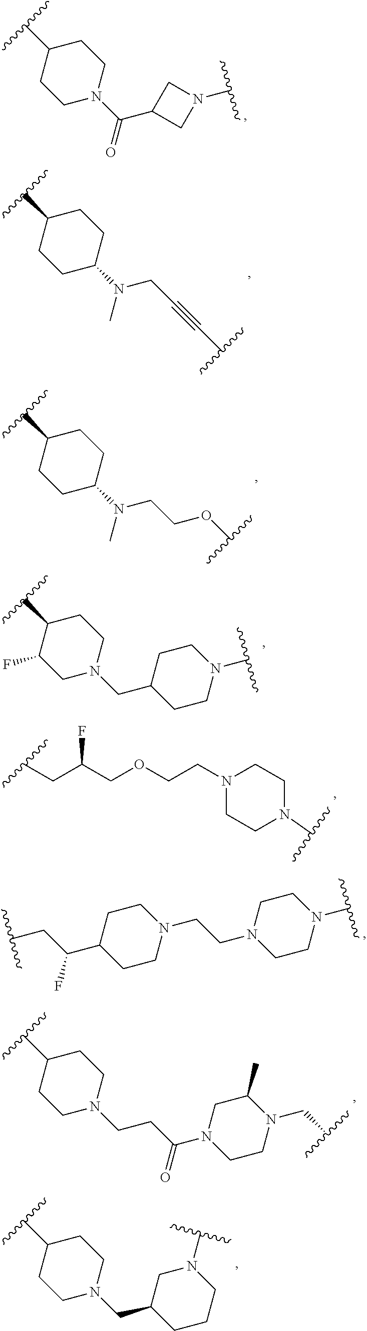

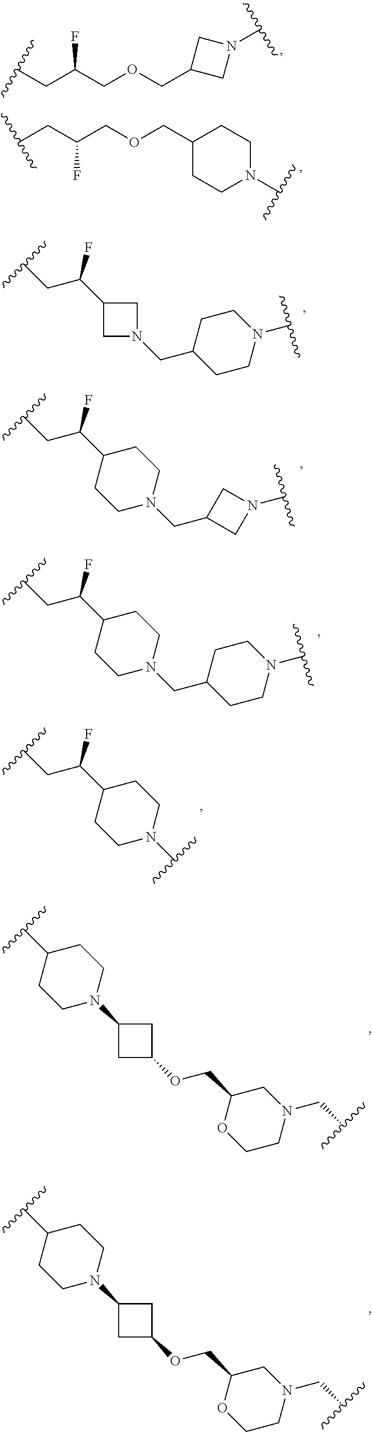

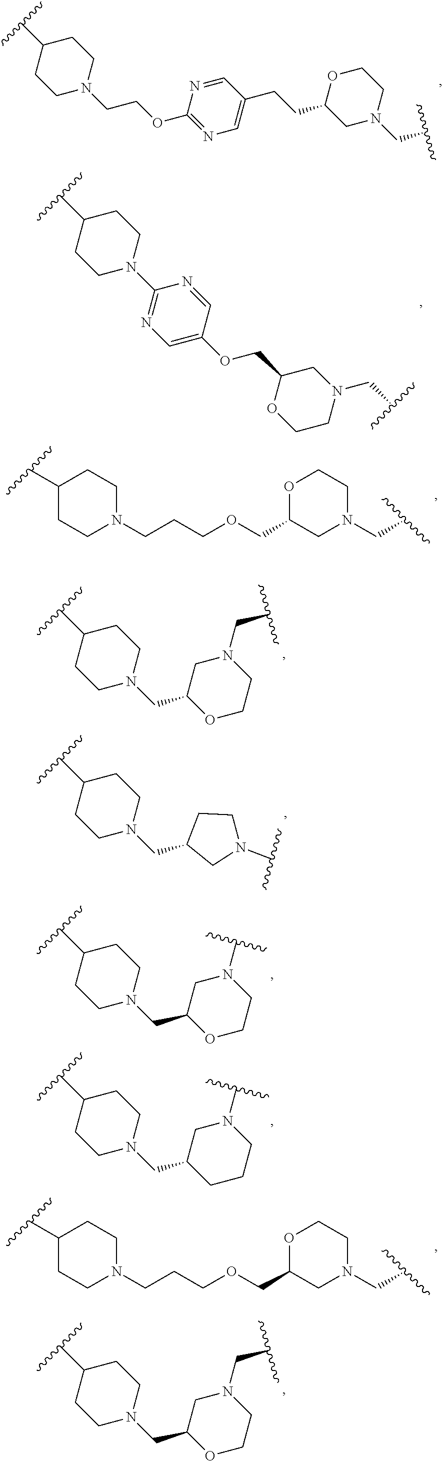

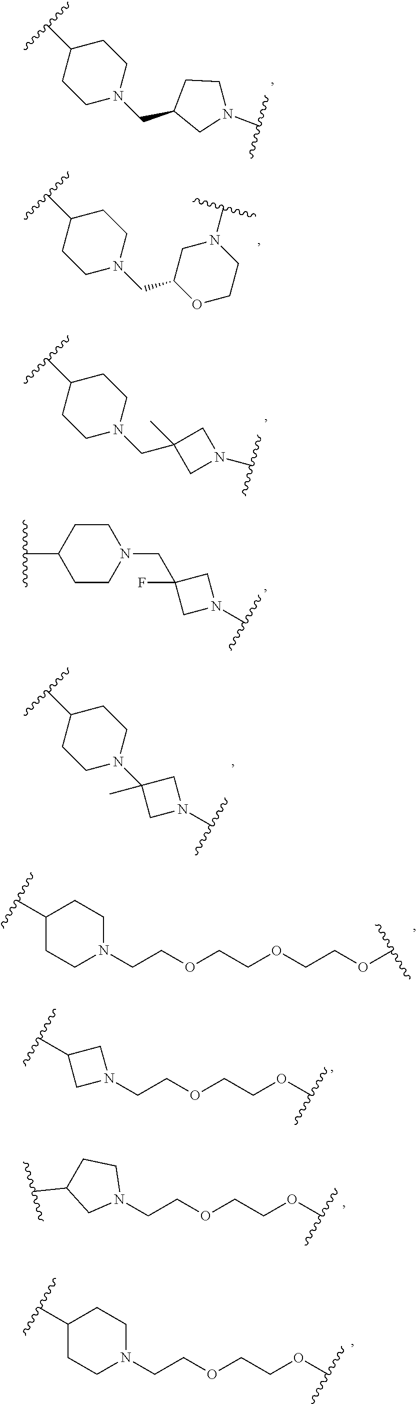

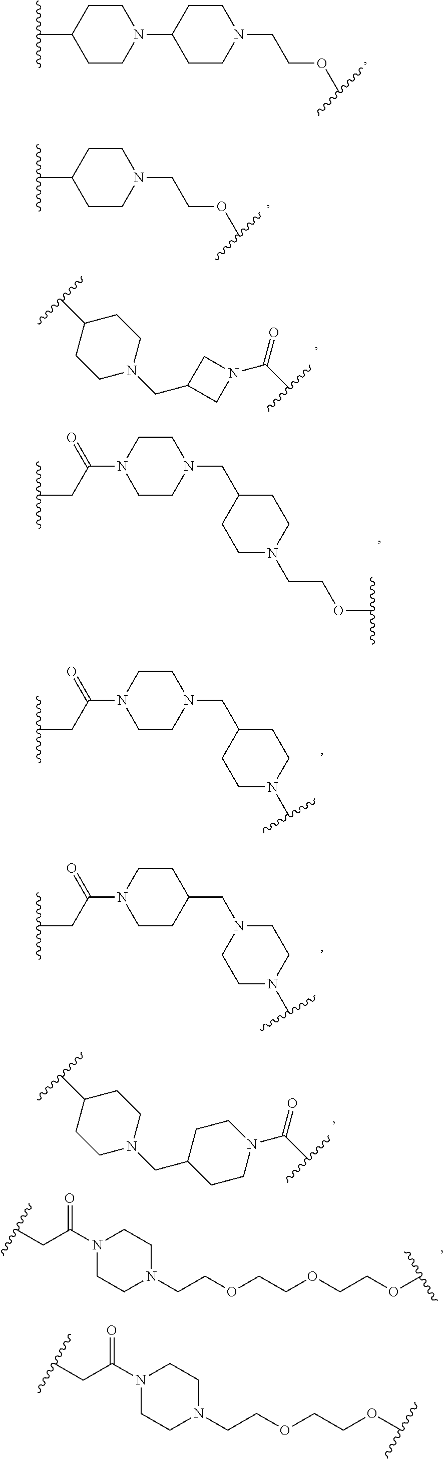

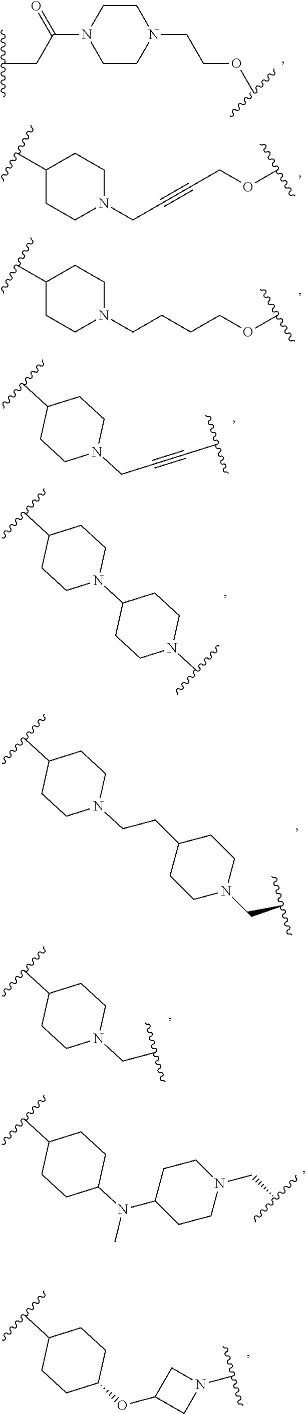

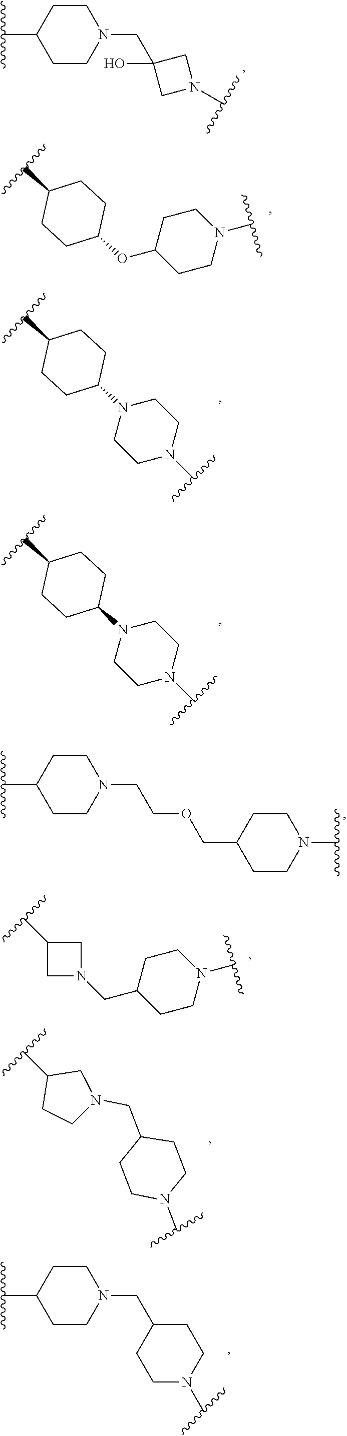

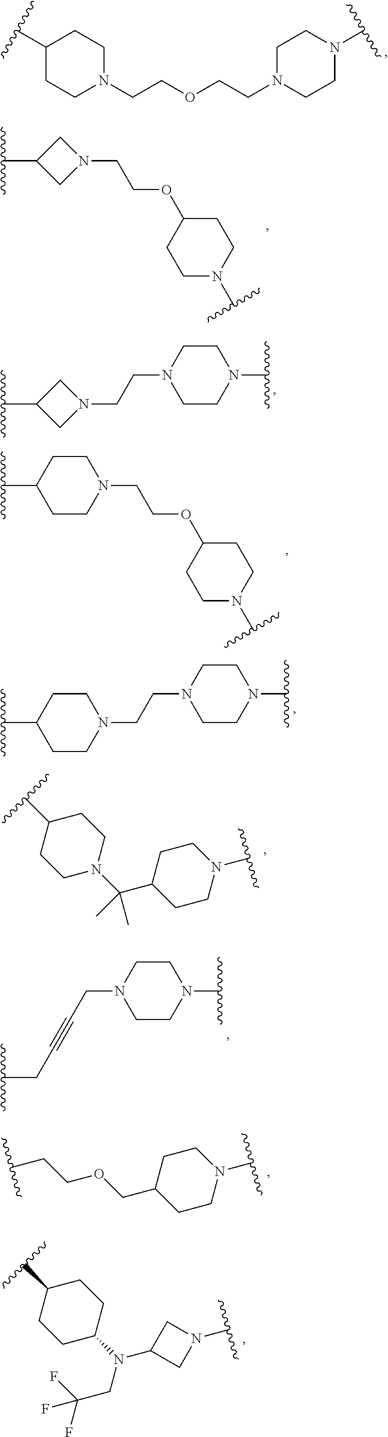

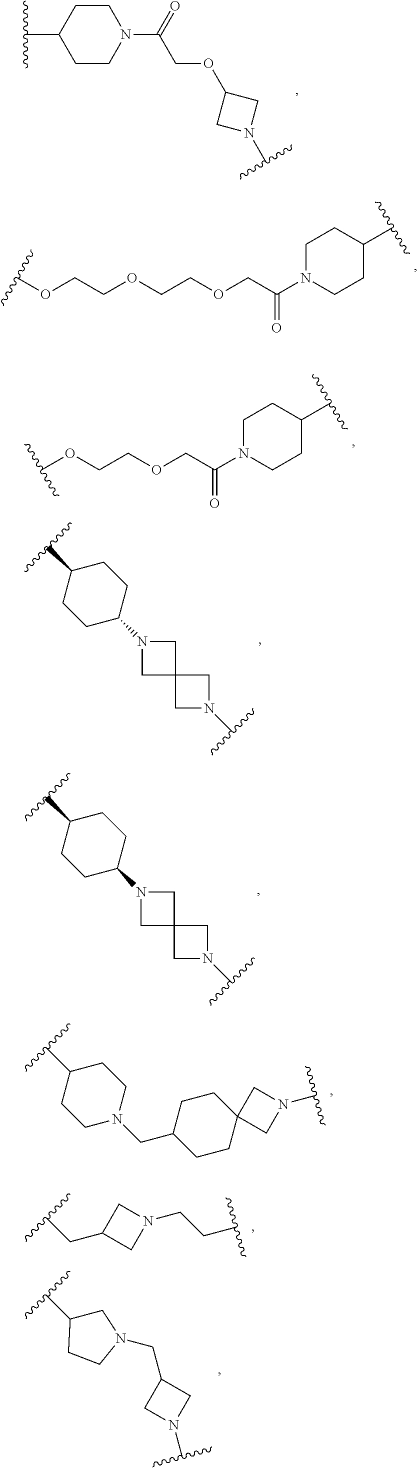

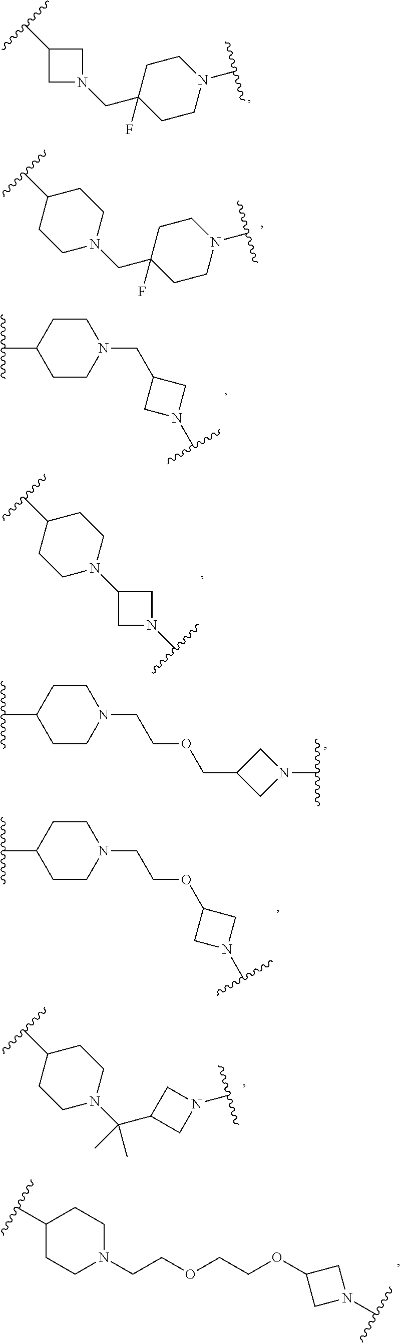

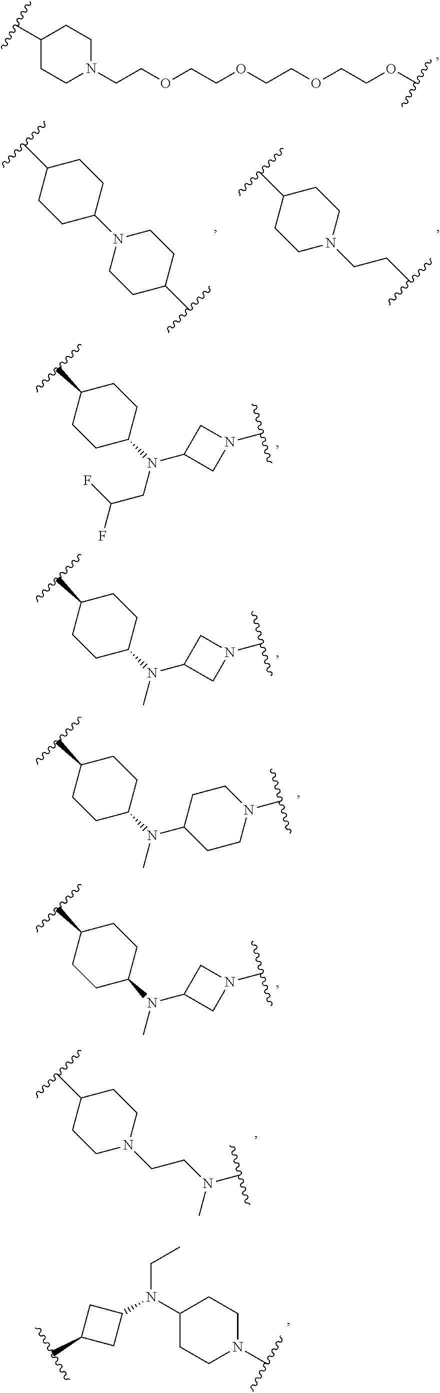

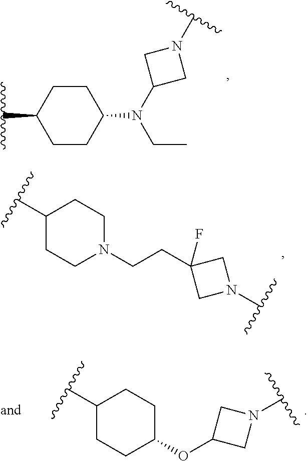

In some embodiments, the right side of the linker as depicted herein connects to the CLM, and the left side of the linker connects to the ITM. In some embodiments, the left side of the linker as depicted herein connects to the CLM, and the right side of the linker connects to the ITM.

In one embodiment, the CLM is a cereblon E3 ubiquitin ligase binding moiety.

In one embodiment, the CLM is a cereblon E3 ubiquitin ligase binding moiety that is CLM-I:

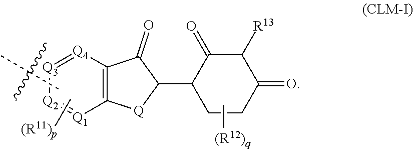

In one embodiment, Q¹, Q², Q³, and Q⁴ are each independently selected from the group consisting of CH, C, and N.

In one embodiment, Q¹, Q², Q³, and Q⁴ are each independently selected from the group consisting of C and N.

In one embodiment, Q¹ is N. In one embodiment, Q² is N. In one embodiment, Q³ is N. In one embodiment, Q⁴ is N.
In one embodiment, Q¹ is C. In one embodiment, Q² is C. In one embodiment, Q³ is C. In one embodiment, Q⁴ is C. In one embodiment, Q¹ is CH. In one embodiment, Q² is CH. In one embodiment, Q³ is CH. In one embodiment, Q⁴ is CH.

In one embodiment, each R¹¹ is independently selected from the group consisting of —Cl, —F, —Br, —I, CN, CF₃, NH₂, NH—(C1-C₆ alkyl), N(C1-C₆ alkyl)(C1-C₆ alkyl), OH, O—C₁₋₆ alkyl optionally substituted with R²¹, and linear or branched C₁₋₆ alkyl optionally substituted with R²¹.

In one embodiment, each R¹¹ is independently selected from the group consisting of —Cl, —F, —Br, —I, CN, and CF₃. In one embodiment, each R¹¹ is —Cl. In one embodiment, each R¹¹ is —F.

In one embodiment, each R¹¹ is —Br. In one embodiment, each R¹¹ is —I. In one embodiment, each R¹¹ is CN. In one embodiment, each R¹¹ is CF₃.

In one embodiment, each R¹¹ is independently selected from the group consisting of NH₂, NH—(C1-C₆ alkyl), and N(C1-C₆ alkyl)(C1-C₆ alkyl). In one embodiment, each R¹¹ is NH₂. In one embodiment, each R¹¹ is NH—(C1-C₆ alkyl). In one embodiment, each R¹¹ is N(C1-C₆ alkyl)(C1-C₆ alkyl).

In one embodiment, each R¹¹ is independently selected from the group consisting of OH, O—C₁₋₆ alkyl, and linear or branched C₁₋₆ alkyl. In one embodiment, each R¹¹ is OH.

In one embodiment, each R¹¹ is O—C₁ alkyl. In one embodiment, each R¹¹ is O—C₂ alkyl. In one embodiment, each R¹¹ is O—C₃ alkyl. In one embodiment, each R¹¹ is O—C₄ alkyl. In one embodiment, each R¹¹ is O—C₅ alkyl. In one embodiment, each R¹¹ is O—C₆ alkyl.

In one embodiment, each R¹¹ is C₁ alkyl. In one embodiment, each R¹¹ is C₂ alkyl. In one embodiment, each R¹¹ is linear or branched C₃ alkyl. In one embodiment, each R¹¹ is linear or branched C₄ alkyl. In one embodiment, each R¹¹ is linear or branched C₅ alkyl. In one embodiment, each R¹¹ is linear or branched C₆ alkyl.

In one embodiment, each R¹¹ is independently selected from the group consisting of O—C₁₋₆ alkyl substituted with R²¹ and linear or branched C₁₋₆ alkyl substituted with R²¹. In one embodiment, each R¹¹ is O—C₁ alkyl substituted with R¹. In one embodiment, each R¹¹ is O—C₂ alkyl substituted with R²¹. In one embodiment, each R¹¹ is O—C3 alkyl substituted with R²¹. In one embodiment, each R¹¹ is O—C₄ alkyl substituted with R¹. In one embodiment, each R¹¹ is O—C₅ alkyl substituted with R²¹. In one embodiment, each R¹¹ is O—C₆ alkyl substituted with R¹¹. In one embodiment, each R¹¹ is C₁ alkyl substituted with R¹. In one embodiment, each R¹¹ is C₂ alkyl substituted with R²¹. In one embodiment, each R¹¹ is linear or branched C₃ alkyl substituted with R²¹. In one embodiment, each R¹¹ is linear or branched C4 alkyl substituted with R²¹. In one embodiment, each R¹¹ is linear or branched C₅ alkyl substituted with R²¹. In one embodiment, each R¹¹ is linear or branched C₆ alkyl substituted with R²¹.

In one embodiment, p is 0. In one embodiment, p is 1. In one embodiment, p is 2. In one embodiment, p is 3. In one embodiment, p is 4.

In one embodiment, Q is selected from the group consisting of CH₂, CH(C₁₋₆ alkyl), C═O, SO₂, NH, and N(C₁₋₆ alkyl). In one embodiment, Q is CH₂. In one embodiment, Q is CH(C₁₋₆ alkyl). In one embodiment, Q is C═O. In one embodiment, Q is SO2. In one embodiment, Q is NH. In one embodiment, Q is N(C₁₋₆ alkyl).

In one embodiment, each R¹² is independently selected from the group consisting of —Cl, —F, —Br, —I, O—C₁₋₆ alkyl optionally substituted with R²¹, NH₂, NH—(C1-C₆ alkyl), N(C1-C₆ alkyl)(C1-C₆ alkyl), OH, CN, CF₃, and linear or branched C₁₋₆ alkyl optionally substituted with R¹.

In one embodiment, each R¹² is independently selected from the group consisting of —Cl, —F, —Br, —I, CN, and CF₃. In one embodiment, each R¹² is —Cl. In one embodiment, each R¹² is —F.

In one embodiment, each R¹² is —Br. In one embodiment, each R¹² is —I. In one embodiment, each R¹² is CN. In one embodiment, each R¹² is CF₃.

In one embodiment, each R¹² is independently selected from the group consisting of NH₂, NH—(C1-C₆ alkyl), and N(C1-C₆ alkyl)(C1-C₆ alkyl). In one embodiment, each R¹² is NH₂. In one embodiment, each R¹² is NH—(C1-C₆ alkyl). In one embodiment, each R¹² is N(C1-C₆ alkyl)(C1-C₆ alkyl)

In one embodiment, each R¹² is independently selected from the group consisting of OH, O—C₁₋₆ alkyl, and linear or branched C₁₋₆ alkyl. In one embodiment, each R¹² is OH.

In one embodiment, each R¹² is O—C₁ alkyl. In one embodiment, each R¹² is O—C₂ alkyl. In one embodiment, each R¹² is O—C₃ alkyl. In one embodiment, each R¹² is O—C₄ alkyl. In one embodiment, each R¹² is O—C₅ alkyl. In one embodiment, each R¹² is O—C₆ alkyl. In one embodiment, each R¹² is C1 alkyl. In one embodiment, each R¹² is C2 alkyl. In one embodiment, each R¹² is linear or branched C₃ alkyl. In one embodiment, each R¹² is linear or branched C₄ alkyl. In one embodiment, each R¹² is linear or branched C₅ alkyl. In one embodiment, each R¹² is linear or branched C₆ alkyl.

In one embodiment, each R¹² is independently selected from the group consisting of O—C₁. 6 alkyl substituted with R²¹ and linear or branched C₁₋₆ alkyl substituted with R²¹. In one embodiment, each R¹² is O—C₁ alkyl substituted with R²¹. In one embodiment, each R¹² is O—C₂ alkyl substituted with R²¹. In one embodiment, each R¹² is O—C₃ alkyl substituted with R²¹. In one embodiment, each R¹² is O—C₄ alkyl substituted with R²¹. In one embodiment, each R¹² is O—C₅ alkyl substituted with R²¹. In one embodiment, each R¹² is O—C₆ alkyl substituted with R²¹. In one embodiment, each R¹² is C₁ alkyl substituted with R²¹. In one embodiment, each R¹² is C₂ alkyl substituted with R²¹. In one embodiment, each R¹² is linear or branched C₃ alkyl substituted with R²¹. In one embodiment, each R¹² is linear or branched C₄ alkyl substituted with R²¹. In one embodiment, each R¹² is linear or branched C₅ alkyl substituted with R²¹. In one embodiment, each R¹² is linear or branched C₆ alkyl substituted with R²¹.

In one embodiment, q is 0. In one embodiment, q is 1. In one embodiment, q is 2. In one embodiment, q is 3.

In one embodiment, R¹³ is selected from the group consisting of H, OH, linear or branched C₁₋₆ alkyl optionally substituted with R²¹, C(O)C₁₋₆ alkyl, C(O)NH—C₁₋₆ alkyl and C(O)OC₁₋₆ alkyl. In one embodiment, R¹³ is H. In one embodiment, R¹³ is OH. In one embodiment, R¹³ is linear or branched C₁₋₆ alkyl. In one embodiment, R¹³ is C1 alkyl. In one embodiment, R¹³ is C₂ alkyl. In one embodiment, R¹³ is linear or branched C₃ alkyl. In one embodiment, R¹³ is linear or branched C₄ alkyl. In one embodiment, R¹³ is linear or branched C₅ alkyl. In one embodiment, R¹³ is linear or branched C₆ alkyl.

In one embodiment, R¹³ is C₁ alkyl substituted with R²¹. In one embodiment, R¹³ is C₂ alkyl substituted with R²¹. In one embodiment, R¹³ is linear or branched C₃ alkyl substituted with R²¹. In one embodiment, R¹³ is linear or branched C₄ alkyl substituted with R²¹. In one embodiment, R¹³ is linear or branched C₅ alkyl substituted with R²¹. In one embodiment, R¹³ is linear or branched C₆ alkyl substituted with R²¹.

In one embodiment, q is 0 and R¹³ is H. In one embodiment, q is 0 and R¹³ is C₁ alkyl.

In one embodiment, q is 1 and R¹³ is H. In one embodiment, q is 1 and R¹³ is C₁ alkyl. In one embodiment, q is 2 and R¹³ is H. In one embodiment, q is 2 and R¹³ is C1 alkyl. In one embodiment, q is 3 and R¹³ is H. In one embodiment, q is 3 and R¹³ is C₁ alkyl.

In one embodiment, each R²¹ is independently selected from the group consisting of —Cl, —F, —OH, NH₂, CN, and CF₃. In one embodiment, R²¹ is —Cl. In one embodiment, R²¹ is —F. In one embodiment, R²¹ is OH. In one embodiment, R²¹ is NH₂. In one embodiment, R²¹ is CN. In one embodiment, R²¹ is CF₃.

In one embodiment, the CLM is a cereblon E3 ubiquitin ligase binding moiety that is CLM-II:

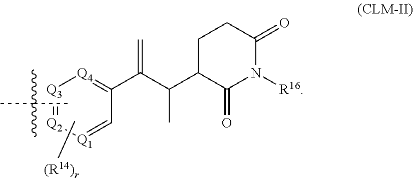

In one embodiment, Q¹, Q², Q³, and Q⁴ are each independently selected from the group consisting of CH, C, and N.

In one embodiment, Q¹, Q², Q³, and Q⁴ are each independently selected from the group consisting of C and N. In one embodiment, Q¹ is N. In one embodiment, Q² is N. In one embodiment, Q³ is N. In one embodiment, Q⁴ is N. In one embodiment, Q¹ is C. In one embodiment, Q² is C. In one embodiment, Q³ is C. In one embodiment, Q⁴ is C. In one embodiment, Q¹ is CH. In one embodiment, Q² is CH. In one embodiment, Q³ is CH.

In one embodiment, Q⁴ is CH. In one embodiment, each R¹⁴ is independently selected from the group consisting of —Cl, —F, —Br, —I, NH₂, NH—(C1-C₆ alkyl), N(C1-C₆ alkyl)(C1-C₆ alkyl), OH, CN, CF₃, O—C₁₋₆ alkyl optionally substituted with R²¹, and linear or branched C₁₋₆ alkyl optionally substituted with R²¹.

In one embodiment, each R¹⁴ is independently selected from the group consisting of —Cl, —F, —Br, —I, CN, and CF₃. In one embodiment, each R¹⁴ is —Cl. In one embodiment, each R¹⁴ is —F. In one embodiment, each R¹⁴ is —Br. In one embodiment, each R¹⁴ is —I. In one embodiment, each R¹⁴ is CN. In one embodiment, each R¹⁴ is CF₃.

In one embodiment, each R¹⁴ is independently selected from the group consisting of NH₂, NH—(C1-C₆ alkyl), and N(C1-C₆ alkyl)(C1-C₆ alkyl). In one embodiment, each R¹⁴ is NH₂. In one embodiment, each R¹⁴ is NH—(C1-C₆ alkyl). In one embodiment, each R¹⁴ is N(C1-C₆ alkyl)(C1-C₆ alkyl). In one embodiment, each R¹⁴ is independently selected from the group consisting of OH, O—C₁₋₆ alkyl, and linear or branched C₁₋₆ alkyl.

In one embodiment, each R¹⁴ is OH. In one embodiment, each R¹⁴ is O—C₁ alkyl. In one embodiment, each R¹⁴ is O—C₂ alkyl. In one embodiment, each R¹⁴ is O—C₃ alkyl. In one embodiment, each R¹⁴ is O—C₄ alkyl. In one embodiment, each R¹⁴ is O—C₅ alkyl. In one embodiment, each R¹⁴ is O—C6 alkyl.

In one embodiment, each R¹⁴ is C₁ alkyl. In one embodiment, each R¹⁴ is C₂ alkyl. In one embodiment, each R¹⁴ is linear or branched C₃ alkyl. In one embodiment, each R¹⁴ is linear or branched C₄ alkyl. In one embodiment, each R¹⁴ is linear or branched C₅ alkyl. In one embodiment, each R¹⁴ is linear or branched C₆ alkyl.

In one embodiment, each R¹⁴ is independently selected from the group consisting of O—C₁₋₆ alkyl optionally substituted with R²¹ and linear or branched C₁₋₆ alkyl optionally substituted with R²¹.

In one embodiment, each R¹⁴ is independently selected from the group consisting of O—C₁₋₆ alkyl substituted with R²¹ and linear or branched C₁₋₆ alkyl substituted with R²¹. In one embodiment, each R¹⁴ is O—C₁ alkyl substituted with R²¹. In one embodiment, each R¹⁴ is O—C₂ alkyl substituted with R²¹. In one embodiment, each R¹⁴ is O—C₃ alkyl substituted with R²¹. In one embodiment, each R¹⁴ is O—C₄ alkyl substituted with R²¹.

In one embodiment, each R¹⁴ is O—C₅ alkyl substituted with R²¹. In one embodiment, each R¹⁴ is O—C₆ alkyl substituted with R²¹.

In one embodiment, each R¹⁴ is C₁ alkyl substituted with R²¹. In one embodiment, each R¹⁴ is C₂ alkyl substituted with R²¹. In one embodiment, each R¹⁴ is linear or branched C₃ alkyl substituted with R²¹. In one embodiment, each R¹⁴ is linear or branched C₄ alkyl substituted with R²¹. In one embodiment, each R¹⁴ is linear or branched C₅ alkyl substituted with R²¹. In one embodiment, each R¹⁴ is linear or branched C₆ alkyl substituted with R²¹.

In one embodiment, r is 0. In one embodiment, r is 1. In one embodiment, r is 2. In one embodiment, r is 3. In one embodiment, r is 4.

In one embodiment, R¹⁵ is selected from the group consisting of H and linear or branched C₁₋₆ alkyl optionally substituted with R²¹. In one embodiment, R¹⁵ is H. In one embodiment, R¹⁵ is linear or branched C₁₋₆ alkyl. In one embodiment, R¹⁵ is C₁ alkyl. In one embodiment, R¹⁵ is C₂ alkyl. In one embodiment, R¹⁵ is linear or branched C3 alkyl. In one embodiment, R¹⁵ is linear or branched C₄ alkyl. In one embodiment, R¹⁵ is linear or branched C₅ alkyl. In one embodiment, R¹⁵ is linear or branched C₆ alkyl.

In one embodiment, R¹⁵ is linear or branched C₁₋₆ alkyl substituted with R²¹. In one embodiment, R¹⁵ is C₁ alkyl substituted with R²¹. In one embodiment, R¹⁵ is C₂ alkyl substituted with R²¹. In one embodiment, R¹⁵ is linear or branched C3 alkyl substituted with R²¹. In one embodiment, R¹⁵ is linear or branched C₄ alkyl substituted with R²¹. In one embodiment, R¹⁵ is linear or branched C₅ alkyl substituted with R²¹. In one embodiment, R¹⁵ is linear or branched C₆ alkyl substituted with R²¹.

In one embodiment, R¹⁶ is selected from the group consisting of H, OH, linear or branched C₁₋₆ alkyl optionally substituted with R²¹, C(O)C₁₋₆ alkyl, C(O)NH—C₁₋₆ alkyl and C(O)OC₁₋₆ alkyl.

In one embodiment, R¹⁶ is H. In one embodiment, R¹⁶ is OH. In one embodiment, R¹⁶ is linear or branched C₁₋₆ alkyl. In one embodiment, R¹⁶ is C1 alkyl. In one embodiment, R¹⁶ is C₂ alkyl. In one embodiment, R¹⁶ is linear or branched C₃ alkyl. In one embodiment, R¹⁶ is linear or branched C₄ alkyl. In one embodiment, R¹⁶ is linear or branched C₅ alkyl. In one embodiment, R¹⁶ is linear or branched C₆ alkyl.

In one embodiment, R¹⁶ is C₁ alkyl substituted with R²¹. In one embodiment, R¹⁶ is C₂ alkyl substituted with R²¹. In one embodiment, R¹⁶ is linear or branched C₃ alkyl substituted with R²¹.

In one embodiment, R¹⁶ is linear or branched C₄ alkyl substituted with R²¹. In one embodiment, R¹⁶ is linear or branched C₅ alkyl substituted with R²¹. In one embodiment, R¹⁶ is linear or branched C6 alkyl substituted with R²¹.

In one embodiment, R¹⁵ is H and R¹⁶ is H. In one embodiment, R¹⁵ is C₁ alkyl and R¹⁶ is H. In one embodiment, R¹⁵ is H and R¹⁶ is C₁ alkyl. In one embodiment, R¹⁵ is C₁ alkyl and R¹⁶ is C₁ alkyl.

In one embodiment, each R²¹ is independently selected from the group consisting of —Cl, —F, —OH, NH₂, CN, and CF₃. In one embodiment, R²¹ is —Cl. In one embodiment, R²¹ is —F. In one embodiment, R²¹ is OH. In one embodiment, R²¹ is NH₂. In one embodiment, R²¹ is CN. In one embodiment, R²¹ is CF₃.

In one embodiment, the CLM is a cereblon E3 ubiquitin ligase binding moiety that is CLM-III:

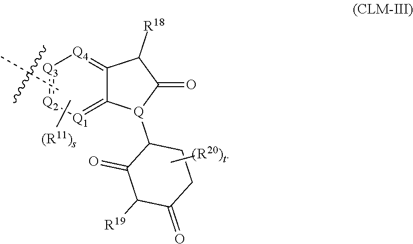

In one embodiment, Q¹, Q², Q³, and Q⁴ are each independently selected from the group consisting of CH, C, and N. In one embodiment, Q¹, Q², Q³, and Q⁴ are each independently selected from the group consisting of C and N. In one embodiment, Q¹ is N. In one embodiment, Q² is N.

In one embodiment, Q³ is N. In one embodiment, Q⁴ is N.

In one embodiment, Q¹ is C. In one embodiment, Q² is C. In one embodiment, Q³ is C. In one embodiment, Q⁴ is C.

In one embodiment, Q¹ is CH. In one embodiment, Q² is CH. In one embodiment, Q³ is CH. In one embodiment, Q⁴ is CH.

In one embodiment, s is 0. In one embodiment, s is 1. In one embodiment, s is 2. In one embodiment, s is 3.

In one embodiment, R¹⁸ is selected from the group consisting of H and linear or branched C₁₋₆ alkyl optionally substituted with R²¹. In one embodiment, R¹⁸ is H. In one embodiment, R¹⁸ is linear or branched C₁₋₆ alkyl. In one embodiment, R¹⁸ is C₁ alkyl. In one embodiment, R¹⁸ is C₂ alkyl. In one embodiment, R¹⁸ is linear or branched C₃ alkyl. In one embodiment, R¹⁸ is linear or branched C₄ alkyl. In one embodiment, R¹⁸ is linear or branched C₅ alkyl. In one embodiment, R¹⁸ is linear or branched C₆ alkyl. In one embodiment, R¹⁸ is linear or branched C₁₋₆ alkyl substituted with R²¹. In one embodiment, R¹⁸ is C₁ alkyl substituted with R²¹. In one embodiment, R¹⁸ is C₂ alkyl substituted with R²¹. In one embodiment, R¹⁸ is linear or branched C₃ alkyl substituted with R²¹. In one embodiment, R¹⁸ is linear or branched C₄ alkyl substituted with R²¹. In one embodiment, R¹⁸ is linear or branched C₅ alkyl substituted with R²¹. In one embodiment, R¹⁸ is linear or branched C₆ alkyl substituted with R²¹.

In one embodiment, R¹⁹ is selected from the group consisting of H, OH, linear or branched C₁₋₆ alkyl optionally substituted with R²¹, C(O)C₁₋₆ alkyl, C(O)NH—C₁₋₆ alkyl and C(O)OC₁₋₆ alkyl. In one embodiment, R¹⁹ is H. In one embodiment, R¹⁹ is OH. In one embodiment, R¹⁹ is linear or branched C₁₋₆ alkyl. In one embodiment, R¹⁹ is C1 alkyl. In one embodiment, R¹⁹ is C₂ alkyl. In one embodiment, R¹⁹ is linear or branched C3 alkyl. In one embodiment, R¹⁹ is linear or branched C₄ alkyl. In one embodiment, R¹⁹ is linear or branched C₅ alkyl. In one embodiment, R¹⁹ is linear or branched C₆ alkyl.

In one embodiment, R¹⁹ is C₁ alkyl substituted with R²¹. In one embodiment, R¹⁹ is C₂ alkyl substituted with R²¹. In one embodiment, R¹⁹ is linear or branched C₃ alkyl substituted with R²¹.

In one embodiment, R¹⁹ is linear or branched C4 alkyl substituted with R²¹. In one embodiment, R¹⁹ is linear or branched C₅ alkyl substituted with R²¹. In one embodiment, R¹⁹ is linear or branched C₆ alkyl substituted with R²¹.

In one embodiment, each R²⁰ is independently selected from the group consisting of —Cl, —F, —Br, —I, O—C₁₋₆ alkyl optionally substituted with R²¹, NH₂, NH—(C₁-C₆ alkyl), N(C₁-C₆ alkyl)(C1-C₆ alkyl), OH, CN, CF₃, and linear or branched C₁₋₆ alkyl optionally substituted with R²¹. In one embodiment, each R²⁰ is independently selected from the group consisting of —Cl, —F, —Br, —I, CN, and CF₃. In one embodiment, each R²⁰ is —Cl. In one embodiment, each R²⁰ is —F. In one embodiment, each R²⁰ is —Br. In one embodiment, each R²⁰ is —I. In one embodiment, each R²⁰ is CN. In one embodiment, each R²⁰ is CF₃.

In one embodiment, each R²⁰ is independently selected from the group consisting of NH₂, NH—(C1-C₆ alkyl), and N(C1-C₆ alkyl)(C₁-C₆ alkyl). In one embodiment, each R²⁰ is NH₂. In one embodiment, each R²⁰ is NH—(C1-C₆ alkyl). In one embodiment, each R²⁰ is N(C1-C₆ alkyl)(C1-C₆ alkyl).

In one embodiment, each R²⁰ is independently selected from the group consisting of OH, O-C1-6 alkyl, and linear or branched C1-6 alkyl. In one embodiment, each R²⁰ is OH.

In one embodiment, each R²⁰ is O—C₁ alkyl. In one embodiment, each R²⁰ is O—C₂ alkyl. In one embodiment, each R²⁰ is O—C₃ alkyl. In one embodiment, each R²⁰ is O—C₄ alkyl. In one embodiment, each R²⁰ is O—C₅ alkyl. In one embodiment, each R²⁰ is O—C₆ alkyl.

In one embodiment, each R²⁰ is C₁ alkyl. In one embodiment, each R²⁰ is C₂ alkyl. In one embodiment, each R²⁰ is linear or branched C₃ alkyl. In one embodiment, each R²⁰ is linear or branched C₄ alkyl. In one embodiment, each R²⁰ is linear or branched C₅ alkyl. In one embodiment, each R²⁰ is linear or branched C₆ alkyl.

In one embodiment, each R²⁰ is independently selected from the group consisting of O—C₁₋₆ alkyl substituted with R²¹ and linear or branched C₁₋₆ alkyl substituted with R²¹. In one embodiment, each R²⁰ is O—C₁ alkyl substituted with R²¹. In one embodiment, each R²⁰ is O—C₂ alkyl substituted with R²¹. In one embodiment, each R²⁰ is O—C3 alkyl substituted with R²¹. In one embodiment, each R²⁰ is O—C₄ alkyl substituted with R²¹. In one embodiment, each R²⁰ is O—C₅ alkyl substituted with R²¹. In one embodiment, each R²⁰ is O—C₆ alkyl substituted with R²¹.

In one embodiment, each R²⁰ is C₁ alkyl substituted with R²¹. In one embodiment, each R²⁰ is C₂ alkyl substituted with R²¹. In one embodiment, each R²⁰ is linear or branched C₃ alkyl substituted with R²¹. In one embodiment, each R²⁰ is linear or branched C4 alkyl substituted with R²¹. In one embodiment, each R²⁰ is linear or branched C₅ alkyl substituted with R²¹. In one embodiment, each R²⁰ is linear or branched C₆ alkyl substituted with R²¹.

In one embodiment, t is 0. In one embodiment, t is 1. In one embodiment, t is 2. In one embodiment, t is 3.

In one embodiment, t is 0 and R¹⁹ is H. In one embodiment, t is 0 and R¹⁹ is C₁ alkyl.

In one embodiment, t is 1 and R¹⁹ is H. In one embodiment, t is 1 and R¹⁹ is C₁ alkyl.

In one embodiment, t is 2 and R¹⁹ is H. In one embodiment, t is 2 and R¹⁹ is C₁ alkyl.

In one embodiment, t is 3 and R¹⁹ is H. In one embodiment, t is 3 and R¹⁹ is C₁ alkyl.

In one embodiment, each R²¹ is independently selected from the group consisting of —Cl, —F, —OH, NH₂, CN, and CF₃. In one embodiment, R²¹ is —Cl. In one embodiment, R²¹ is —F. In one embodiment, R²¹ is OH. In one embodiment, R²¹ is NH₂. In one embodiment, R²¹ is CN. In one embodiment, R²¹ is CF₃.

In one embodiment, the CLM is selected from the group consisting of:

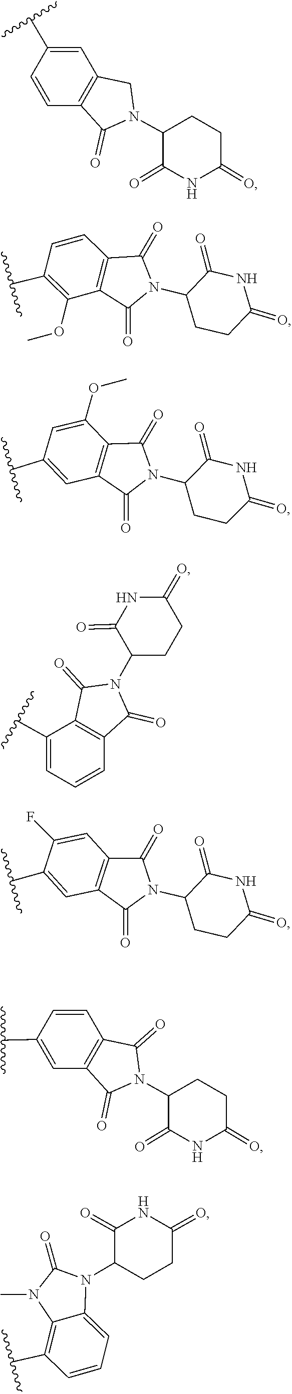

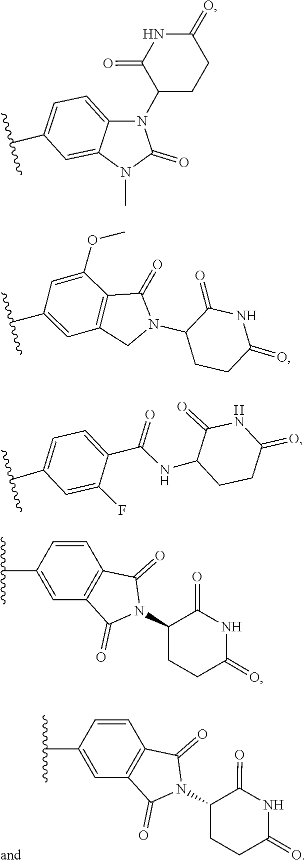

In one embodiment, the bifunctional compound of the application has an ITM that is:

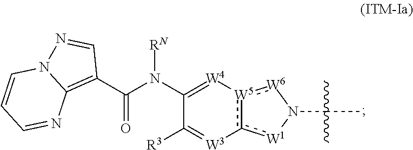

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

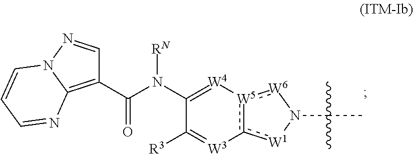

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

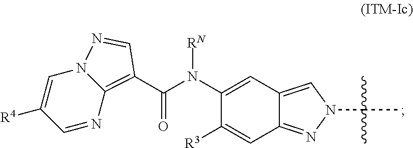

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

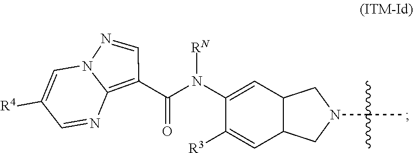

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

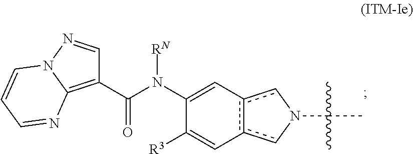

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

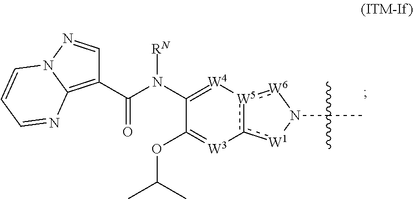

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

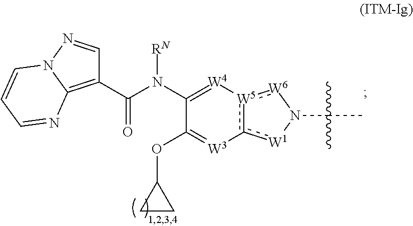

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

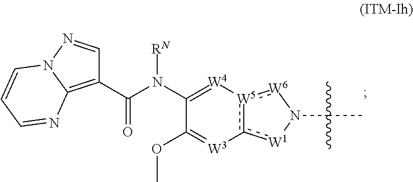

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

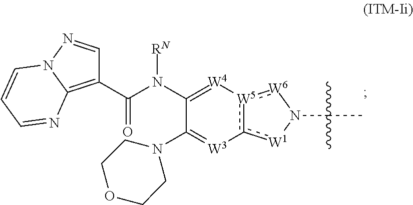

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

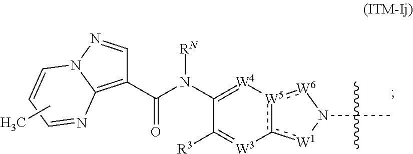

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

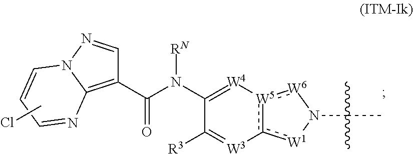

and a LNK that is selected from the group consisting of Z—Y—Z—, —Z—X—Z—Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

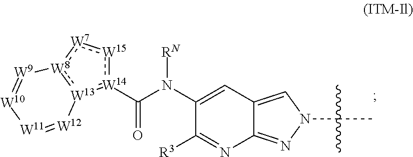

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

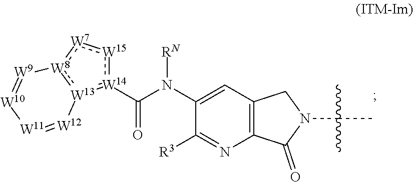

and a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, wherein all variables are defined as set forth herein.

In one embodiment, the bifunctional compound of the application has an ITM that is:

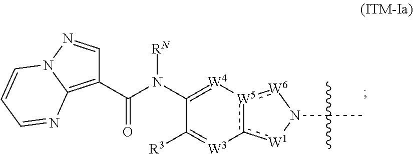

and a CLM that is CLM-I, wherein all variables are defined as set forth herein.

In one embodiment, the bifunctional compound of the application has an ITM that is:

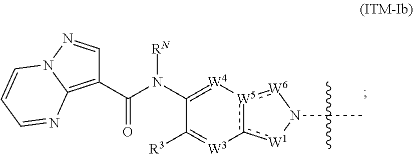

and a CLM that is CLM-I, wherein all variables are defined as set forth herein.

In one embodiment, the bifunctional compound of the application has an ITM that is:

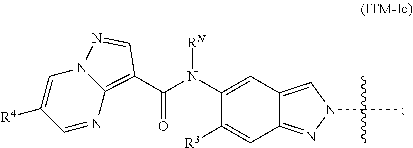

and a CLM that is CLM-I, wherein all variables are defined as set forth herein.

In one embodiment, the bifunctional compound of the application has an ITM that is:

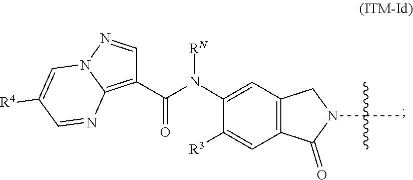

and a CLM that is CLM-I, wherein all variables are defined as set forth herein.

In one embodiment, the bifunctional compound of the application has an ITM that is:

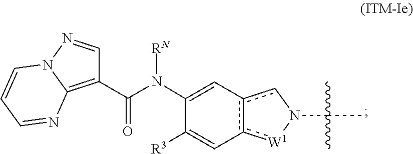

and a CLM that is CLM-I, wherein all variables are defined as set forth herein.

In one embodiment, the bifunctional compound of the application has an ITM that is:

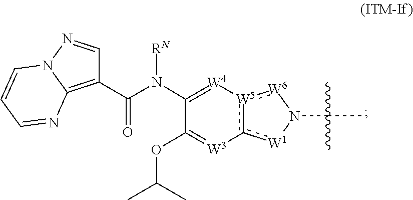

and a CLM that is CLM-I, wherein all variables are defined as set forth herein.

In one embodiment, the bifunctional compound of the application has an ITM that is:

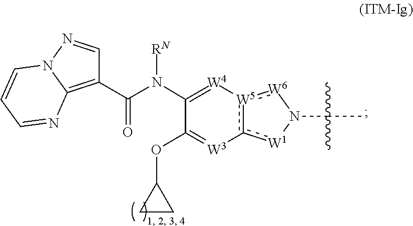

and a CLM that is CLM-I, wherein all variables are defined as set forth herein.

In one embodiment, the bifunctional compound of the application has an ITM that is:

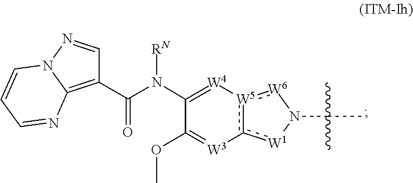

and a CLM that is CLM-I, wherein all variables are defined as set forth herein.

In one embodiment, the bifunctional compound of the application has an ITM that is:

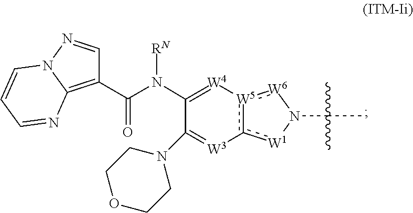

and a CLM that is CLM-I, wherein all variables are defined as set forth herein.

In one embodiment, the bifunctional compound of the application has an ITM that is:

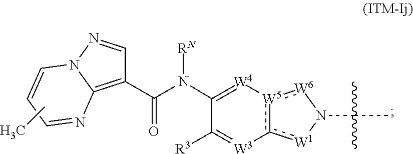

and a CLM that is CLM-I, wherein all variables are defined as set forth herein.

In one embodiment, the bifunctional compound of the application has an ITM that is:

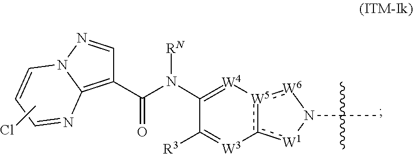

and a CLM that is CLM-I, wherein all variables are defined as set forth herein.
In one embodiment, the bifunctional compound of the application has an ITM that is:

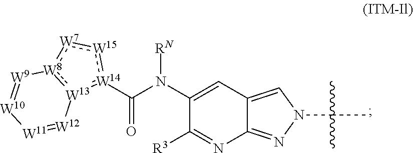

and a CLM that is CLM-I, wherein all variables are defined as set forth herein.

In one embodiment, the bifunctional compound of the application has an ITM that is:

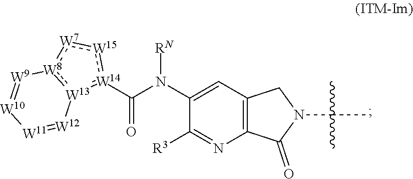

and a CLM that is CLM-I, wherein all variables are defined as set forth herein.

In one embodiment, the bifunctional compound of the application has an ITM that is:

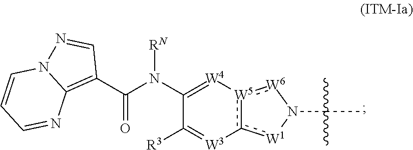

and a CLM that is CLM-II, wherein all variables are defined as set forth herein.

In one embodiment, the bifunctional compound of the application has an ITM that is:

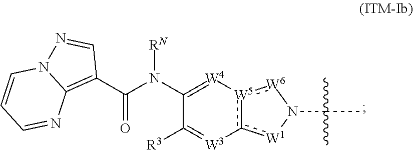

and a CLM that is CLM-II, wherein all variables are defined as set forth herein.

In one embodiment, the bifunctional compound of the application has an ITM that is:

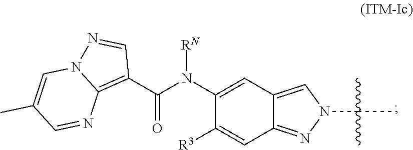

and a CLM that is CLM-II, wherein all variables are defined as set forth herein.

In one embodiment, the bifunctional compound of the application has an ITM that is:

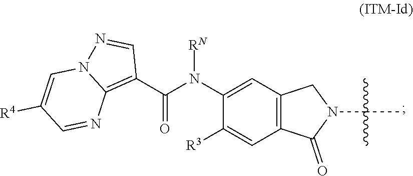

and a CLM that is CLM-II, wherein all variables are defined as set forth herein.

In one embodiment, the bifunctional compound of the application has an ITM that is:

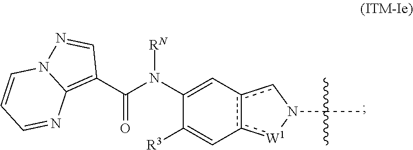

and a CLM that is CLM-II, wherein all variables are defined as set forth herein.

In one embodiment, the bifunctional compound of the application has an ITM that is:

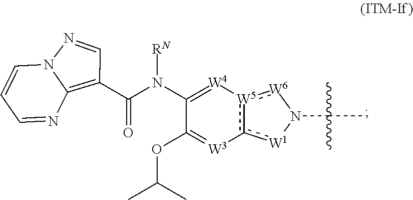

and a CLM that is CLM-II, wherein all variables are defined as set forth herein.

In one embodiment, the bifunctional compound of the application has an ITM that is:

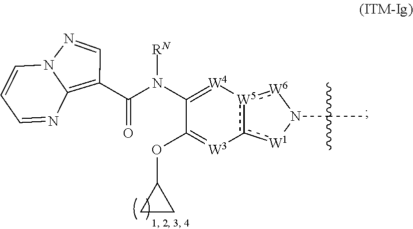

and a CLM that is CLM-II, wherein all variables are defined as set forth herein.

In one embodiment, the bifunctional compound of the application has an ITM that is:

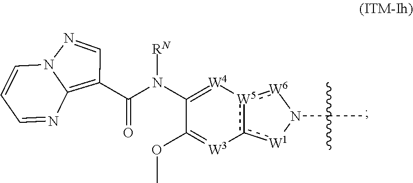

and a CLM that is CLM-II, wherein all variables are defined as set forth herein.

In one embodiment, the bifunctional compound of the application has an ITM that is:

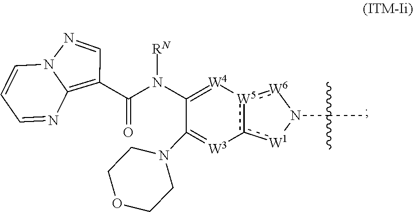

and a CLM that is CLM-II, wherein all variables are defined as set forth herein.

In one embodiment, the bifunctional compound of the application has an ITM that is:

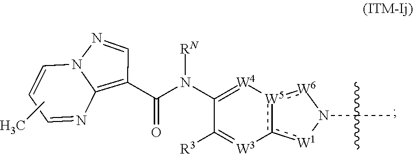

and a CLM that is CLM-II, wherein all variables are defined as set forth herein.

In one embodiment, the bifunctional compound of the application has an ITM that is:

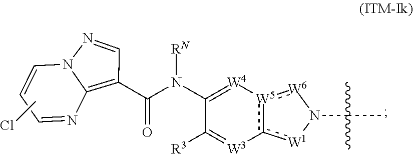

and a CLM that is CLM-II, wherein all variables are defined as set forth herein.

In one embodiment, the bifunctional compound of the application has an ITM that is:

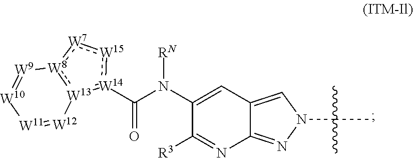

and a CLM that is CLM-II, wherein all variables are defined as set forth herein.

In one embodiment, the bifunctional compound of the application has an ITM that is:

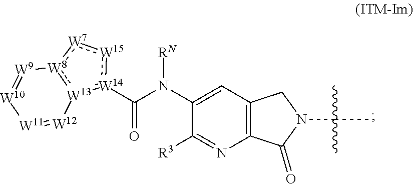

and a CLM that is CLM-II, wherein all variables are defined as set forth herein.

In one embodiment, the bifunctional compound of the application has an ITM that is:

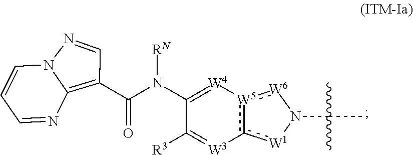

and a CLM that is CLM-III, wherein all variables are defined as set forth herein.

In one embodiment, the bifunctional compound of the application has an ITM that is:

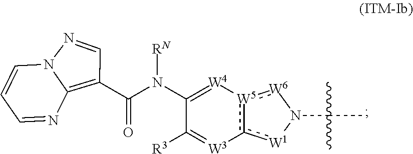

and a CLM that is CLM-III, wherein all variables are defined as set forth herein.

In one embodiment, the bifunctional compound of the application has an ITM that is:

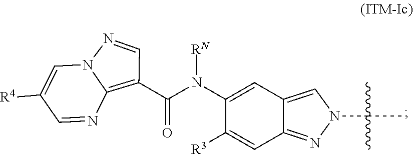

and a CLM that is CLM-III, wherein all variables are defined as set forth herein.

In one embodiment, the bifunctional compound of the application has an ITM that is:

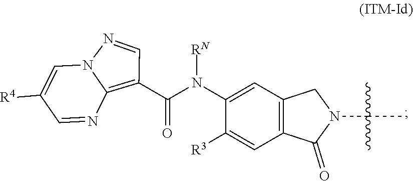

and a CLM that is CLM-III, wherein all variables are defined as set forth herein.

In one embodiment, the bifunctional compound of the application has an ITM that is:

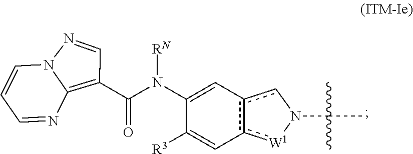

and a CLM that is CLM-III, wherein all variables are defined as set forth herein.

In one embodiment, the bifunctional compound of the application has an ITM that is:

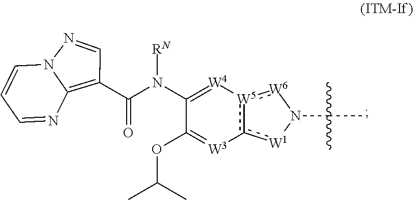

and a CLM that is CLM-III, wherein all variables are defined as set forth herein.

In one embodiment, the bifunctional compound of the application has an ITM that is:

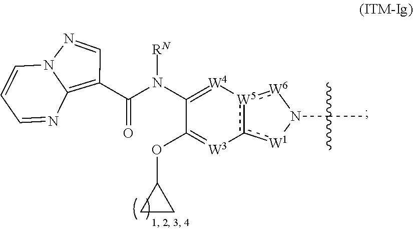

and a CLM that is CLM-III, wherein all variables are defined as set forth herein.

In one embodiment, the bifunctional compound of the application has an ITM that is:

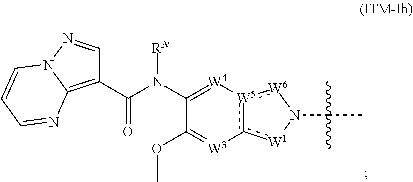

and a CLM that is CLM-III, wherein all variables are defined as set forth herein.

In one embodiment, the bifunctional compound of the application has an ITM that is:

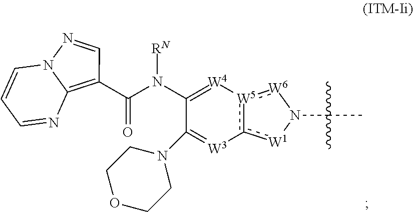

and a CLM that is CLM-III, wherein all variables are defined as set forth herein.

In one embodiment, the bifunctional compound of the application has an ITM that is:

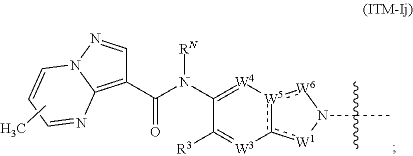

and a CLM that is CLM-III, wherein all variables are defined as set forth herein.

In one embodiment, the bifunctional compound of the application has an ITM that is:

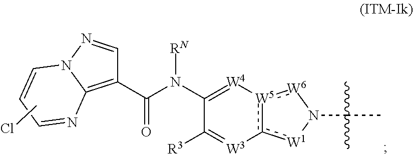

and a CLM that is CLM-III, wherein all variables are defined as set forth herein.

In one embodiment, the bifunctional compound of the application has an ITM that is:

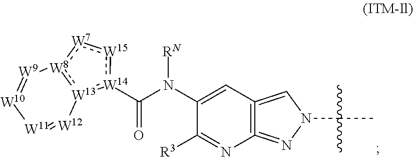

and a CLM that is CLM-III, wherein all variables are defined as set forth herein.

In one embodiment, the bifunctional compound of the application has an ITM that is:

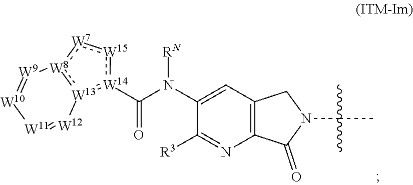

and a CLM that is CLM-III, wherein all variables are defined as set forth herein.

In one embodiment, the bifunctional compound of the application has a LNK that is Z—Y—Z—; and a CLM that is CLM-I, wherein all variables are defined as set forth herein.

In one embodiment, the bifunctional compound of the application has a LNK that is Z—Y—Z—; and a CLM that is CLM-II, wherein all variables are defined as set forth herein.

In one embodiment, the bifunctional compound of the application has a LNK that is Z—Y—Z—; and a CLM that is CLM-III, wherein all variables are defined as set forth herein.

In one embodiment, the bifunctional compound of the application has a LNK that is —Z—X—Z—; and a CLM that is CLM-I, wherein all variables are defined as set forth herein.

In one embodiment, the bifunctional compound of the application has a LNK that is —Z—X—Z—; and a CLM that is CLM-II, wherein all variables are defined as set forth herein.

In one embodiment, the bifunctional compound of the application has a LNK that is —Z—X—Z—; and a CLM that is CLM-III, wherein all variables are defined as set forth herein.

In one embodiment, the bifunctional compound of the application has a LNK that is —Z—X—Z—X—; and a CLM that is CLM-I, wherein all variables are defined as set forth herein.

In one embodiment, the bifunctional compound of the application has a LNK that is selected from the group consisting of —Z—X—Z—X—; and a CLM that is CLM-II, wherein all variables are defined as set forth herein.

In one embodiment, the bifunctional compound of the application has a LNK that is —Z—X—Z—X—; and a CLM that is CLM-III, wherein all variables are defined as set forth herein.

In one embodiment, the bifunctional compound of the application has a LNK that is —Z—X—Y—; and a CLM that is CLM-I, wherein all variables are defined as set forth herein.

In one embodiment, the bifunctional compound of the application has a LNK that is —Z—X—Y—; and a CLM that is CLM-II, wherein all variables are defined as set forth herein.

In one embodiment, the bifunctional compound of the application has a LNK that is —Z—X—Y—; and a CLM that is CLM-III, wherein all variables are defined as set forth herein.

In one embodiment, the bifunctional compound of the application has a LNK that is —Y—Z—Y—Z—; and a CLM that is CLM-I, wherein all variables are defined as set forth herein.

In one embodiment, the bifunctional compound of the application has a LNK that is —Y—Z—Y—Z—; and a CLM that is CLM-II, wherein all variables are defined as set forth herein.

In one embodiment, the bifunctional compound of the application has a LNK that is —Y—Z—Y—Z—; and a CLM that is CLM-III, wherein all variables are defined as set forth herein.

In one embodiment, the bifunctional compound of the application has a LNK that is —Y—Z—X—Z—; and a CLM that is CLM-I, wherein all variables are defined as set forth herein.

In one embodiment, the bifunctional compound of the application has a LNK that is —Y—Z—X—Z—; and a CLM that is CLM-II, wherein all variables are defined as set forth herein.

In one embodiment, the bifunctional compound of the application has a LNK that is —Y—Z—X—Z—; and a CLM that is CLM-III, wherein all variables are defined as set forth herein.

In one embodiment, the bifunctional compound of the application has a LNK that is —Z—Z—; and a CLM that is CLM-I, wherein all variables are defined as set forth herein.

In one embodiment, the bifunctional compound of the application has a LNK that is —Z—Z—; and a CLM that is CLM-II, wherein all variables are defined as set forth herein.

In one embodiment, the bifunctional compound of the application has a LNK that is —Z—Z—; and a CLM that is CLM-III, wherein all variables are defined as set forth herein.

In one embodiment, the bifunctional compound of the application has a LNK that is —(X)₀₋₁—(Y)₁₋₅—Z—; and a CLM that is CLM-I, wherein all variables are defined as set forth herein.

In one embodiment, the bifunctional compound of the application has a LNK that is —(X)₀₋₁—(Y)₁₋₅—Z—; and a CLM that is CLM-II, wherein all variables are defined as set forth herein.

In one embodiment, the bifunctional compound of the application has a LNK that is —(X)₀₋₁—(Y)₁₋₅—Z—; and a CLM that is CLM-III, wherein all variables are defined as set forth herein.

In one embodiment, the bifunctional compound of the application has a LNK that is —X—Z—(Y)₁₋₄—; and a CLM that is CLM-I, wherein all variables are defined as set forth herein.

In one embodiment, the bifunctional compound of the application has a LNK that is —X—Z—(Y)₁₋₄—; and a CLM that is CLM-II, wherein all variables are defined as set forth herein.

In one embodiment, the bifunctional compound of the application has a LNK that is —X—Z—(Y)₁₋₄—; and a CLM that is CLM-III, wherein all variables are defined as set forth herein.

In one embodiment, the bifunctional compound of the application has a LNK that is —Z—(Y)₁₋₅—; and a CLM that is CLM-I, wherein all variables are defined as set forth herein.

In one embodiment, the bifunctional compound of the application has a LNK that is —Z—(Y)₁₋₅—; and a CLM that is CLM-II, wherein all variables are defined as set forth herein.

In one embodiment, the bifunctional compound of the application has a LNK that is —Z—(Y)₁₋₅—; and a CLM that is CLM-III, wherein all variables are defined as set forth herein.

In one embodiment, the bifunctional compound of the application has an ITM that is ITM-Ja, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I wherein all variables are defined as set forth herein.

In one embodiment, the bifunctional compound of the application has an ITM that is ITM-Ib, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has and ITM that is ITM-Ic, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has an ITM that is ITM-Id, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has ITM that is ITM-Ie, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has an ITM that is ITM-If, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has an ITM that is ITM-Ig, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has an ITM that is ITM-Jh, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has an ITM that is ITM-Ii, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has an ITM that is ITM-Jj, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has an ITM that is ITM-Jk, a LNK that is selected from the group consisting of Z—Y—Z—, —Z—X—Z—Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has an ITM that is ITM-Il, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has an ITM that is ITM-Im, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-I, wherein all variables are defined as set forth herein.

In one embodiment, the bifunctional compound of the application has an ITM that is ITM-Ia, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has an ITM that is ITM-Ib, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has and ITM that is ITM-Ic, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has an ITM that is ITM-Id, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has ITM that is ITM-Ie, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has an ITM that is ITM-If, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has an ITM that is ITM-Ig, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has an ITM that is ITM-Ih, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has an ITM that is ITM-Ii, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has an ITM that is ITM-Jj, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has an ITM that is ITM-Jk, a LNK that is selected from the group consisting of Z—Y—Z—, —Z—X—Z—Z—X—Z—X—, —Z—X—Y—, —Y—Z—Y—Z—, —Y—Z—X—Z—, —Z—Z—, —(X)₀₋₁-(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has an ITM that is ITM-Il, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has an ITM that is ITM-Im, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-II, wherein all variables are defined as set forth herein.

In one embodiment, the bifunctional compound of the application has an ITM that is ITM-Ia, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has an ITM that is ITM-Ib, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has and ITM that is ITM-Ic, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has an ITM that is ITM-Id, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has ITM that is ITM-Je, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has an ITM that is ITM-If, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has an ITM that is ITM-Ig, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has an ITM that is ITM-Ih, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has an ITM that is ITM-Ii, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has an ITM that is ITM-Ij, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has an ITM that is ITM-Jk, a LNK that is selected from the group consisting of Z—Y—Z—, —Z—X—Z—Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has an ITM that is ITM-Il, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—, and a CLM that is CLM-III, wherein all variables are defined as set forth herein.

In one embodiment, the bifunctional compound of the application has an ITM that is ITM-Im, a LNK that is selected from the group consisting of Z—Y—Z—, —Z—X—Z—, —Z—X—Z—X—, —Z—X—Y—, —Y—Z—Y—Z—, —Y—Z—X—Z—, —Z—Z—, —(X)₀₋₁-(Y)₁₋₅—Z—, —X—Z—(Y)₁₋₄—, and —Z—(Y)₁₋₅—, and a CLM that is CLM-III, wherein all variables are defined as set forth herein.

In another embodiment, the bifunctional compound of Formula (I) has the following structure:

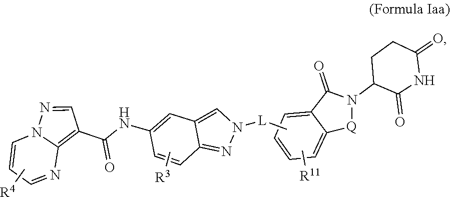

or a pharmaceutically acceptable salt thereof, wherein:
R³ and R⁴ are each independently selected from the group consisting of H, —Cl, —F, —Br, —I, linear or branched C₁₋₆ alkyl optionally substituted with one, two, three, four, or five R⁷, OC₁₋₆ alkyl optionally substituted with one, two, three, four, or five R⁷, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C₃₋₇ cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, OC₃₋₇ heterocycloalkyl optionally substituted with one, two, three, four, or five R⁷, and C₁₋₆alkyl-C₃₋₇cycloalkyl optionally substituted with one, two, three, four, or five R⁷;
each R⁷ is independently selected from the group consisting of —Cl, —F, —Br, —I, NH₂, CN, CF₃, linear or branched C₁₋₆ alkyl, OC₁₋₆ alkyl, NH(C₁-C₆ alkyl), N(C₁₋₆ alkyl)(C1-6 alkyl), C₆-C₁₀ aryl, 5- to 6-membered heteroaryl, C₃-C₇ cycloalkyl, and 3- to 7-membered heterocycloalkyl;
R¹¹ is independently selected from the group consisting of —H, —Cl, —F, —Br, —I, CN, CF₃, NH₂, NH—(C₁-C₆ alkyl), N(C₁-C₆ alkyl)(C1-C₆ alkyl), OH, 0-C1-6 alkyl optionally substituted with R²¹, and linear or branched C₁₋₆ alkyl optionally substituted with R²¹;
each R²¹ is independently selected from the group consisting of —Cl, —F, —OH, NH₂, CN, and CF₃;
Q is selected from the group consisting of CH₂, CH(C₁₋₆ alkyl), C═O, SO₂, NH, and N(C₁₋₆ alkyl); and
L is selected from

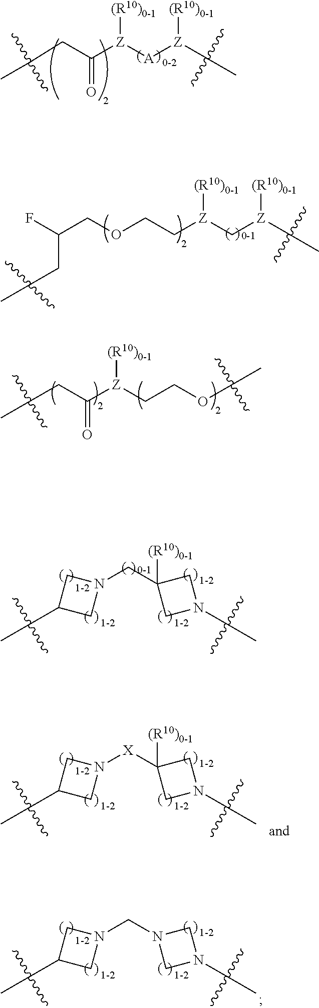

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and —OH;
each A is independently selected from the group consisting of CH₂, NH, and O;
X is selected from the group consisting of CH₂, NH, and O; and
Z is selected from the group consisting of CH₂, CH, C, NH, N, and O.

In an embodiment of Formula (Iaa),
R³ and R⁴ are each independently selected from the group consisting of —H, —Cl, —F, —Br, —I, linear or branched C₁₋₆ alkyl optionally substituted with one, two, three, four, or five R⁷, and OC₁₋₆ alkyl optionally substituted with one, two, three, four, or five R⁷, and OC₃₋₇ cycloalkyl optionally substituted with one, two, three, four, or five R⁷;
each R⁷ is independently selected from the group consisting of —Cl, —F, —Br, —I, linear or branched C₁₋₆ alkyl, OC₁₋₆ alkyl, and C₃-C₇ cycloalkyl;
R¹¹ is independently selected from the group consisting of —H, —Cl, —F, —Br, —I, O—C₁₋₆ alkyl, and linear or branched C₁₋₆ alkyl;
Q is selected from the group consisting of CH₂, C═O; and
L is selected from the group consisting of

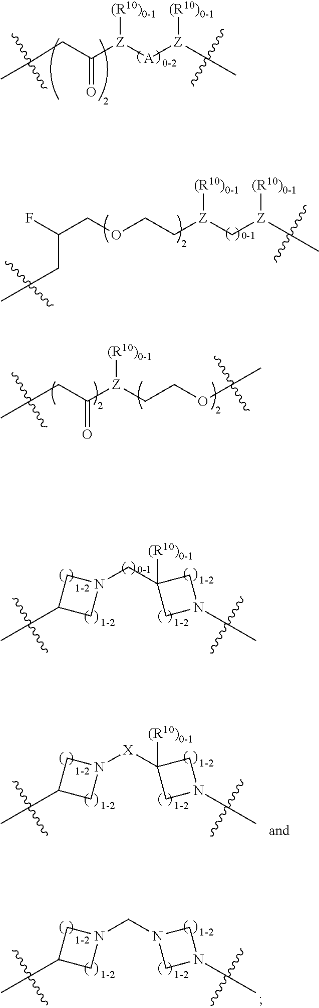

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and —OH;
each A is independently selected from the group consisting of CH₂, NH, and O;
X is selected from CH₂, NH, and O; and
Z is selected from the group consisting of CH₂, CH, C, NH, N, and O.

In another embodiment of Formula (Iaa),
R³ and R⁴ are each independently selected from the group consisting of —H and OC₁₋₆ alkyl optionally substituted with one, two, three, four, or five R⁷;
R⁷ is C₃-C₇ cycloalkyl;
R¹¹ is selected from the group consisting of —H, —Cl, —F, —Br, —I, O—C₁₋₆ alkyl, and linear or branched C₁₋₆ alkyl;
Q is selected from the group consisting of CH₂ and C═O; and
L is

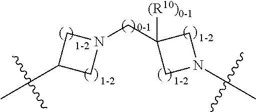

wherein:
R¹⁰ is selected from the group consisting of —Cl, —F, —Br, and —I.

In another embodiment, the bifunctional compound of Formula (I) has the following structure:

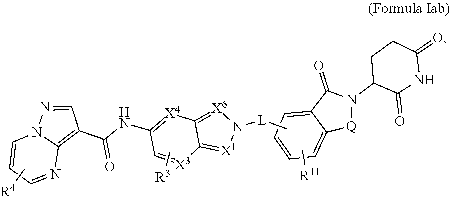

or a pharmaceutically acceptable salt thereof, wherein:
X¹ and X⁶ are each independently C═O, C(R¹)₂, or NR¹;
X³ and X⁴ are each independently N or CR¹;
R¹ is H, —Cl, —F, —Br, —I, N(R²)₂; linear or branched C₁₋₆ alkyl, C₁₋₆ haloalkyl, 3- to 7-membered heterocycloalkyl, or 5- to 6-membered heteroaryl optionally substituted with one, two, three, four, or five R⁶;
R³ and R⁴ are each independently selected from the group consisting of —H, —Cl, —F, —Br, —I, linear or branched C₁₋₆ alkyl optionally substituted with one, two, three, four, or five R⁷, OC₁₋₆ alkyl optionally substituted with one, two, three, four, or five R⁷, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C₃₋₇ cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, OC₃₋₇ heterocycloalkyl optionally substituted with one, two, three, four, or five R⁷, and C₁₋₆alkyl-C₃₋₇cycloalkyl optionally substituted with one, two, three, four, or five R⁷;
each R⁶ is independently selected from the group consisting of OH, OC₁₋₆ alkyl, and heterocycloalkyl;
each R⁷ is independently selected from the group consisting of —Cl, —F, —Br, —I, NH₂, CN, CF₃, linear or branched C₁₋₆ alkyl, OC₁₋₆ alkyl, NH(C1-C₆ alkyl), N(C₁₋₆ alkyl)(C₁₋₆ alkyl), C₆-C₁₀ aryl, 5- to 6-membered heteroaryl, C₃-C₇ cycloalkyl, and 3- to 7-membered heterocycloalkyl;
R¹¹ is independently selected from the group consisting of —H, —Cl, —F, —Br, —I, CN, CF₃, NH₂, NH—(C₁-C₆ alkyl), N(C₁-C₆ alkyl)(C1-C₆ alkyl), OH, O—C₁₋₆ alkyl optionally substituted with R²¹, and linear or branched C₁₋₆ alkyl optionally substituted with R²¹;
each R²¹ is independently selected from the group consisting of —Cl, —F, —OH, NH₂, CN, and CF₃;
Q is selected from the group consisting of CH₂, CH(C₁₋₆ alkyl), C═O, SO₂, NH, and N(C₁₋₆ alkyl); and
L is selected from the group consisting of

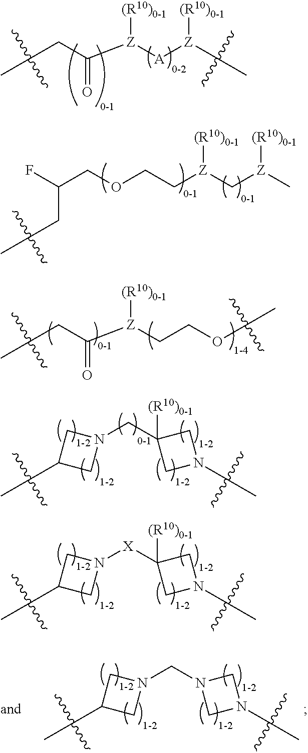

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH;
each A is independently selected from the group consisting of CH₂, NH, and O;
X is selected from the group consisting of CH₂, NH, and O; and
Z is selected from the group consisting of CH₂, CH, C, NH, N, and O.

In an embodiment of Formula (Iab),
X¹ and X⁶ are each independently C═O or CH₂;
X³ and X⁴ are each independently N or CH;
R³ and R⁴ are each independently selected from the group consisting of —H, —Cl, —F, —Br, —I, linear or branched C₁₋₆ alkyl optionally substituted with one, two, three, four, or five R⁷, and OC₁. 6 alkyl, OC₃₋₇ cycloalkyl optionally substituted with one, two, three, four, or five R⁷;
each R⁷ is independently selected from the group consisting of —Cl, —F, —Br, —I, linear or branched C₁₋₆ alkyl, OC₁₋₆ alkyl, and C₃-C₇ cycloalkyl;
R¹¹ is independently selected from the group consisting of —H, —Cl, —F, —Br, —I, O—C₁₋₆ alkyl, and linear or branched C₁₋₆ alkyl;
Q is selected from the group consisting of CH₂, C═O; and
L is selected from the group consisting of

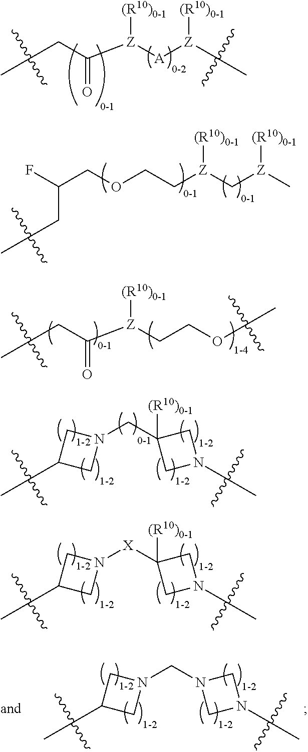

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH;
each A is independently selected from the group consisting of CH₂, NH, and O;
X is selected from the group consisting of CH₂, NH, and O; and
Z is selected from the group consisting of the group consisting of CH₂, CH, C, NH, N, and O.

In another embodiment of Formula (Iab),
X¹ and X⁶ are each independently the group consisting of C═O or CH₂;
X³ and X⁴ are each CH;
R³ and R⁴ are each independently selected from the group consisting of —H and OC₁₋₆ alkyl;
R¹¹ is —H;
Q is selected from the group consisting of CH₂ and C═O; and
L is

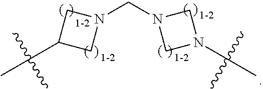 [SMILES: *C1CN(CN2CN(*)C2)C1]

In another embodiment, the bifunctional compound of Formula (I) has the following structure:

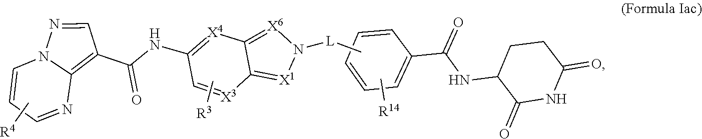

or a pharmaceutically acceptable salt thereof, wherein:
X¹ and X⁶ are each independently C═O, C(R¹)₂, or NR¹;
X³ and X⁴ are each independently N or CR¹;
R¹ is H, —Cl, —F, —Br, —I, N(R²)₂; linear or branched C₁₋₆ alkyl, C₁₋₆ haloalkyl, 3- to 7-membered heterocycloalkyl, or 5- to 6-membered heteroaryl optionally substituted with one, two, three, four, or five R⁶;
R³ and R⁴ are each independently selected from the group consisting of —H, —Cl, —F, —Br, —I, linear or branched C₁₋₆ alkyl optionally substituted with one, two, three, four, or five R⁷, OC₁₋₆ alkyl optionally substituted with one, two, three, four, or five R⁷, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C₃₋₇ cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, OC₃₋₇ heterocycloalkyl optionally substituted with one, two, three, four, or five R⁷, and C₁₋₆alkyl-C₃₋₇cycloalkyl optionally substituted with one, two, three, four, or five R⁷;
each R⁶ is independently selected from the group consisting of OH, OC₁₋₆ alkyl, and heterocycloalkyl;
each R⁷ is independently selected from the group consisting of —Cl, —F, —Br, —I, NH₂, CN, CF₃, linear or branched C₁₋₆ alkyl, OC₁₋₆ alkyl, NH(C1-C₆ alkyl), N(C₁₋₆ alkyl)(C1-6 alkyl), C₆-C₁₀ aryl, 5- to 6-membered heteroaryl, C₃-C₇ cycloalkyl, and 3- to 7-membered heterocycloalkyl;
R¹¹ is independently selected from the group consisting of —H, —Cl, —F, —Br, —I, CN, CF₃, NH₂, NH—(C1-C₆ alkyl), N(C1-C₆ alkyl)(C1-C₆ alkyl), OH, O—C₁₋₆ alkyl optionally substituted with R²¹, and linear or branched C₁₋₆ alkyl optionally substituted with R²¹;
R¹⁴ is selected from the group consisting of —H, —Cl, —F, —Br, —I, NH₂, NH—(C1-C₆ alkyl), N(C1-C₆ alkyl)(C1-C₆ alkyl), OH, CN, CF₃, O—C₁₋₆ alkyl optionally substituted with R²¹, and linear or branched C₁₋₆ alkyl optionally substituted with R²¹;
each R²¹ is independently selected from the group consisting of —Cl, —F, —OH, NH₂, CN, and CF₃;
Q is selected from CH₂, CH(C₁₋₆ alkyl), C═O, SO₂, NH, and N(C₁₋₆ alkyl); and
L is selected from the group consisting of

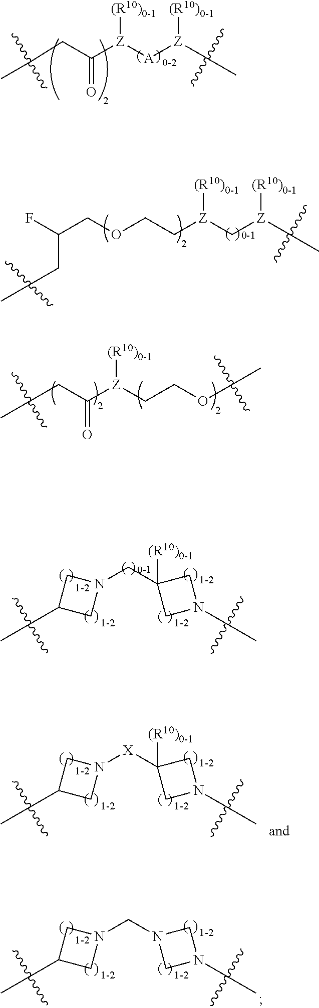

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH;
each A is independently selected from the group consisting of CH₂, NH, and O;
X is selected from CH₂, NH, and O; and
Z is selected from the group consisting of CH₂, CH, C, NH, N, and O.

In an embodiment of Formula (Iac),
X¹ and X⁶ are each independently C═O or CH₂;
X³ and X⁴ are each independently N or CH;
R³ and R⁴ are each independently selected from the group consisting of —H, —Cl, —F, —Br, —I, linear or branched C₁₋₆ alkyl optionally substituted with one, two, three, four, or five R⁷, and OC₁. 6 alkyl, OC₃₋₇ cycloalkyl optionally substituted with one, two, three, four, or five R⁷;
each R⁷ is independently selected from the group consisting of —Cl, —F, —Br, —I, linear or branched C₁₋₆ alkyl, OC₁₋₆ alkyl, and C₃-C₇ cycloalkyl;
R¹¹ is independently selected from the group consisting of —H, —Cl, —F, —Br, —I, O—C₁₋₆ alkyl, and linear or branched C₁₋₆ alkyl;
R¹⁴ is H;
Q is selected from CH₂ and C═O; and
L is selected from the group consisting of

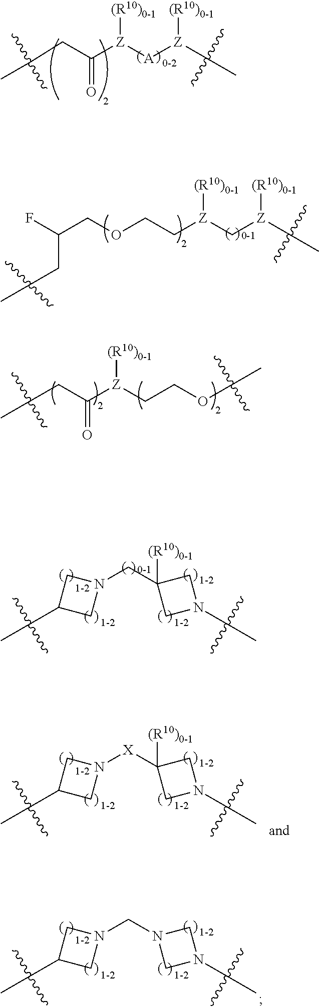

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH;
each A is independently selected from the group consisting of CH₂, NH, and O;
X is selected from the group consisting of CH₂, NH, and O; and
Z is selected from the group consisting of CH₂, CH, C, NH, N, and O.

In another embodiment, the bifunctional compound of Formula (I) has the following structure:

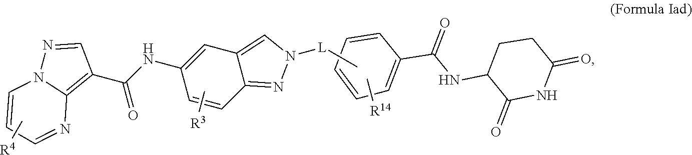

or a pharmaceutically acceptable salt thereof, wherein:
R³ and R⁴ are each independently selected from the group consisting of —H, —Cl, —F, —Br, —I, linear or branched C₁₋₆ alkyl optionally substituted with one, two, three, four, or five R⁷, OC₁₋₆ alkyl optionally substituted with one, two, three, four, or five R⁷, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C₃₋₇ cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, OC₃₋₇ heterocycloalkyl optionally substituted with one, two, three, four, or five R⁷, and C₁₋₆alkyl-C₃₋₇cycloalkyl optionally substituted with one, two, three, four, or five R⁷;
each R⁷ is independently selected from the group consisting of —Cl, —F, —Br, —I, NH₂, CN, CF₃, linear or branched C₁₋₆ alkyl, OC₁₋₆ alkyl, NH(C1-C₆ alkyl), N(C₁₋₆ alkyl)(C₁₋₆ alkyl), C₆-C₁₀ aryl, 5- to 6-membered heteroaryl, C₃-C₇ cycloalkyl, and 3- to 7-membered heterocycloalkyl;
R¹¹ is independently selected from the group consisting of —H, —Cl, —F, —Br, —I, CN, CF₃, NH₂, NH—(C₁-C₆ alkyl), N(C1-C₆ alkyl)(C1-C₆ alkyl), OH, O—C₁₋₆ alkyl optionally substituted with R²¹, and linear or branched C₁₋₆ alkyl optionally substituted with R²¹;
R¹⁴ is selected from the group consisting of —H, —Cl, —F, —Br, —I, NH₂, NH—(C1-C₆ alkyl), N(C1-C₆ alkyl)(C1-C₆ alkyl), OH, CN, CF₃, O—C₁₋₆ alkyl optionally substituted with R²¹, and linear or branched C₁₋₆ alkyl optionally substituted with R²¹;
each R²¹ is independently selected from the group consisting of —Cl, —F, —OH, NH₂, CN, and CF₃;
Q is selected from CH₂, CH(C₁₋₆ alkyl), C═O, SO₂, NH, and N(C₁₋₆ alkyl); and
L is selected from

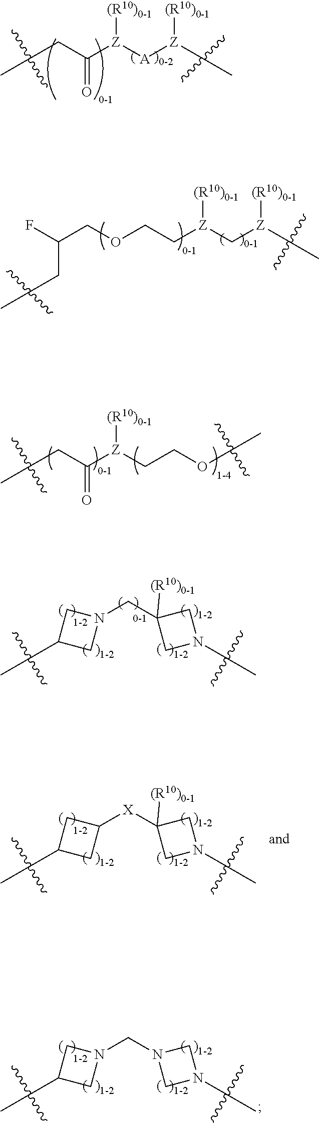

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH;
each A is independently selected from the group consisting of CH₂, NH, and O;
X is selected from the group consisting of CH₂, NH, and O; and
Z is selected from the group consisting of CH₂, CH, C, NH, N, and O.

In an embodiment of Formula (lad),
R³ and R⁴ are each independently selected from the group consisting of —H, —Cl, —F, —Br, —I, linear or branched C₁₋₆ alkyl optionally substituted with one, two, three, four, or five R⁷, and OC₁₋₆ alkyl, OC₃₋₇ cycloalkyl optionally substituted with one, two, three, four, or five R⁷;
each R⁷ is independently selected from the group consisting of —Cl, —F, —Br, —I, linear or branched C₁₋₆ alkyl, OC₁₋₆ alkyl, and C3-C7 cycloalkyl;
R¹¹ is independently selected from the group consisting of —H, —Cl, —F, —Br, —I, O—C₁₋₆ alkyl, and linear or branched C₁₋₆ alkyl;
R¹⁴ is selected from the group consisting of —H, O—C₁₋₆ alkyl, and linear or branched C₁₋₆ alkyl;
Q is selected from the group consisting of CH₂, C═O; and
L is selected from the group consisting of

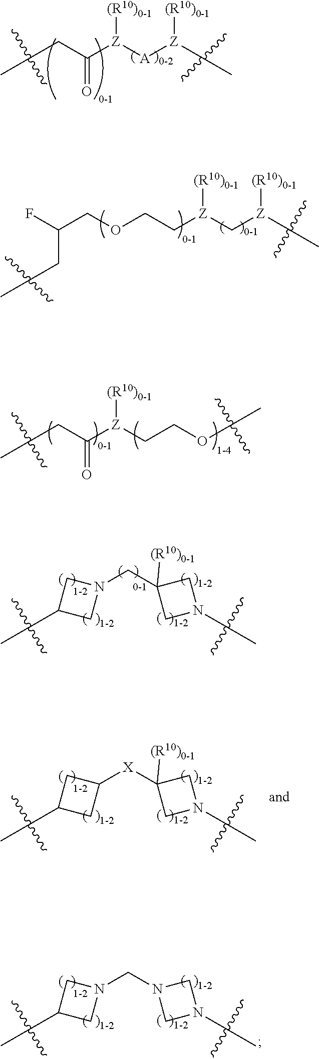

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH;
each A is independently selected from the group consisting of CH₂, NH, and O;
X is selected from the group consisting of CH₂, NH, and O; and
Z is selected from the group consisting of CH₂, CH, C, NH, N, and O.

In another embodiment, the bifunctional compound of Formula (I) has the following structure:

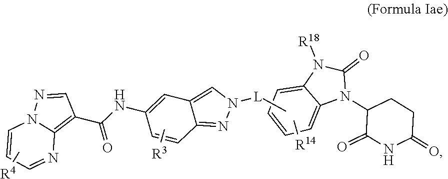

or a pharmaceutically acceptable salt thereof, wherein:
R³ and R⁴ are each independently selected from the group consisting of —H, —Cl, —F, —Br, —I, linear or branched C₁₋₆ alkyl optionally substituted with one, two, three, four, or five R⁷, OC₁₋₆ alkyl optionally substituted with one, two, three, four, or five R⁷, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C₃₋₇ cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, OC₃₋₇ heterocycloalkyl optionally substituted with one, two, three, four, or five R⁷, and C₁₋₆alkyl-C₃₋₇cycloalkyl optionally substituted with one, two, three, four, or five R⁷;
each R⁷ is independently selected from the group consisting of —Cl, —F, —Br, —I, NH₂, CN, CF₃, linear or branched C₁₋₆ alkyl, OC₁₋₆ alkyl, NH(C1-C₆ alkyl), N(C1-6 alkyl)(C1-6 alkyl), C6-C₁₀ aryl, 5- to 6-membered heteroaryl, C₃-C₇ cycloalkyl, and 3- to 7-membered heterocycloalkyl;
R¹¹ is independently selected from the group consisting of —H, —Cl, —F, —Br, —I, CN, CF₃, NH₂, NH—(C₁-C₆ alkyl), N(C1-C₆ alkyl)(C₁-C₆ alkyl), OH, O—C₁₋₆ alkyl optionally substituted with R²¹, and linear or branched C₁₋₆ alkyl optionally substituted with R²¹;
R¹⁴ is selected from the group consisting of —H, —Cl, —F, —Br, —I, NH₂, NH—(C1-C₆ alkyl), N(C1-C₆ alkyl)(C1-C₆ alkyl), OH, CN, CF₃, O—C₁₋₆ alkyl optionally substituted with R²¹, and linear or branched C₁₋₆ alkyl optionally substituted with R²¹;
R¹⁸ is selected from the group consisting of —H, C₁₋₆ alkyl, and C₁₋₆ haloalkyl;
each R²¹ is independently selected from the group consisting of —Cl, —F, —OH, NH₂, CN, and CF₃;
Q is selected from the group consisting of CH₂, CH(C₁₋₆ alkyl), C═O, SO₂, NH, and N(C₁₋₆ alkyl); and
L is selected from the group consisting of

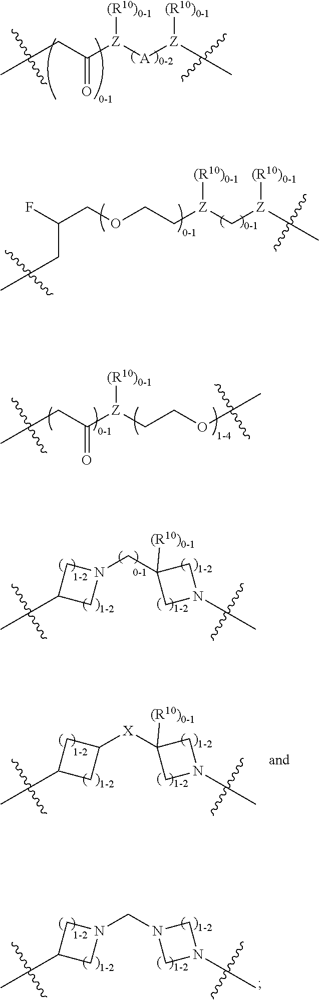

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH;
each A is independently selected from the group consisting of CH₂, NH, and O;
X is selected from the group consisting of CH₂, NH, and O; and
Z is selected from the group consisting of CH₂, CH, C, NH, N, and O.

In an embodiment of Formula (Iae),
R³ and R⁴ are each independently selected from the group consisting of —H, —Cl, —F, —Br, —I, linear or branched C₁₋₆ alkyl optionally substituted with one, two, three, four, or five R⁷, and OC₁₋₆ alkyl, OC₃₋₇ cycloalkyl optionally substituted with one, two, three, four, or five R⁷;
each R⁷ is independently selected from the group consisting of —Cl, —F, —Br, —I, linear or branched C₁₋₆ alkyl, OC₁₋₆ alkyl, and C₃-C₇ cycloalkyl;
R¹¹ is independently selected from the group consisting of —H, —Cl, —F, —Br, —I, O—C₁₋₆ alkyl, and linear or branched C₁₋₆ alkyl;
R¹⁴ is selected from the group consisting of —H and linear or branched C1-6 alkyl;
R¹⁸ is selected from the group consisting of —H and C₁₋₆ alkyl;
Q is selected from the group consisting of CH₂, C═O; and
L is selected from the group consisting of

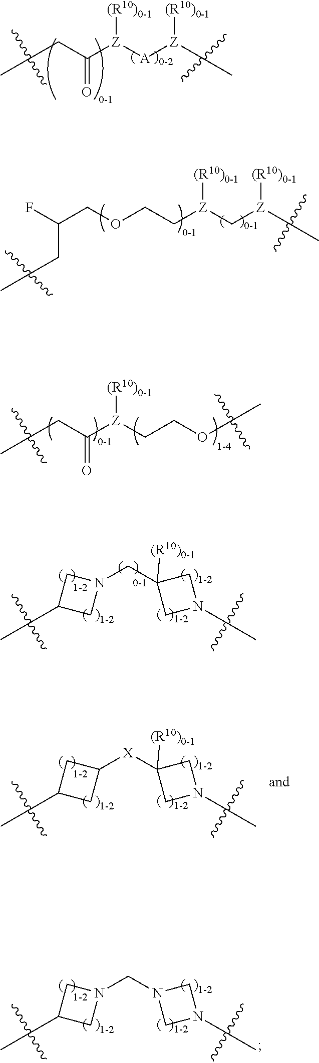

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH;
each A is independently selected from the group consisting of CH₂, NH, and O;
X is selected from the group consisting of CH₂, NH, and O; and
Z is selected from the group consisting of CH₂, CH, C, NH, N, and O.

In another embodiment, the bifunctional compound of Formula (I) has the following structure:

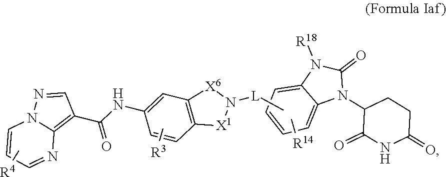

or a pharmaceutically acceptable salt thereof, wherein:
X¹ and X⁶ are each independently C═O, C(R¹)₂, or NR¹;
R¹ is H, —Cl, —F, —Br, —I, N(R²)₂; linear or branched C₁₋₆ alkyl, C₁₋₆ haloalkyl, 3- to 7-membered heterocycloalkyl, or 5- to 6-membered heteroaryl optionally substituted with one, two, three, four, or five R⁶;
R³ and R⁴ are each independently selected from the group consisting of —H, —Cl, —F, —Br, —I, linear or branched C₁₋₆ alkyl optionally substituted with one, two, three, four, or five R⁷, OC₁₋₆ alkyl optionally substituted with one, two, three, four, or five R⁷, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C₃₋₇ cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, OC₃₋₇ heterocycloalkyl optionally substituted with one, two, three, four, or five R⁷, and C₁₋₆alkyl-C₃₋₇cycloalkyl optionally substituted with one, two, three, four, or five R⁷;
each R⁶ is independently selected from the group consisting of OH, OC₁₋₆ alkyl, and heterocycloalkyl;
each R⁷ is independently selected from the group consisting of —Cl, —F, —Br, —I, NH₂, CN, CF₃, linear or branched C₁₋₆ alkyl, OC₁₋₆ alkyl, NH(C1-C₆ alkyl), N(C₁₋₆ alkyl)(C1-6 alkyl), C₆-C₁₀ aryl, 5- to 6-membered heteroaryl, C₃-C₇ cycloalkyl, and 3- to 7-membered heterocycloalkyl;
R¹¹ is independently selected from the group consisting of —H, —Cl, —F, —Br, —I, CN, CF₃, NH₂, NH—(C₁-C₆ alkyl), N(C1-C₆ alkyl)(C1-C₆ alkyl), OH, O—C₁₋₆ alkyl optionally substituted with R²¹, and linear or branched C₁₋₆ alkyl optionally substituted with R²¹;
R¹⁴ is selected from the group consisting of —H, —Cl, —F, —Br, —I, NH₂, NH—(C1-C₆ alkyl), N(C1-C₆ alkyl)(C1-C₆ alkyl), OH, CN, CF₃, O—C₁₋₆ alkyl optionally substituted with R²¹, and linear or branched C1-6 alkyl optionally substituted with R²¹;
R¹⁸ is selected from the group consisting of —H, C₁₋₆ alkyl, and C₁₋₆ haloalkyl;
each R²¹ is independently selected from the group consisting of —Cl, —F, —OH, NH₂, CN, and CF₃;
Q is selected from the group consisting of CH₂, CH(C₁₋₆ alkyl), C═O, SO₂, NH, and N(C₁₋₆ alkyl); and
L is selected from the group consisting of

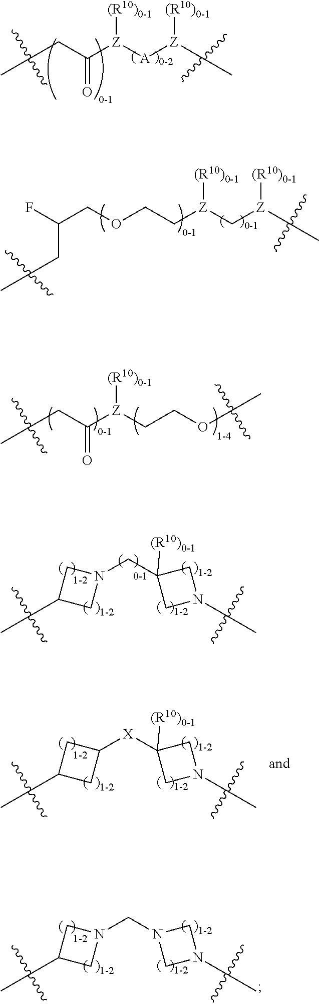

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH;
each A is independently selected from the group consisting of CH₂, NH, and O;
X is selected from the group consisting of CH₂, NH, and O; and
Z is selected from the group consisting of CH₂, CH, C, NH, N, and O.

In an embodiment of Formula (Iaf),
X¹ and X⁶ are each independently C═O or CH₂;
X³ and X⁴ are each independently N or CH;
R³ and R⁴ are each independently selected from the group consisting of —H, —Cl, —F, —Br, —I, linear or branched C₁₋₆ alkyl optionally substituted with one, two, three, four, or five R⁷, and OC₁₋₆ alkyl, OC₃₋₇ cycloalkyl optionally substituted with one, two, three, four, or five R⁷;
each R⁷ is independently selected from the group consisting of —Cl, —F, —Br, —I, linear or branched C₁₋₆ alkyl, OC₁₋₆ alkyl, and C₃-C₇ cycloalkyl;
R¹¹ is independently selected from the group consisting of —H, —Cl, —F, —Br, —I, O—C1-6 alkyl, and linear or branched C₁₋₆ alkyl;
R¹⁴ is selected from the group consisting of —H, O—C₁₋₆ alkyl, and linear or branched C₁₋₆ alkyl;
R¹⁸ is selected from the group consisting of —H and C₁₋₆ alkyl;
Q is selected from the group consisting of CH₂ and C═O; and
L is selected from the group consisting of

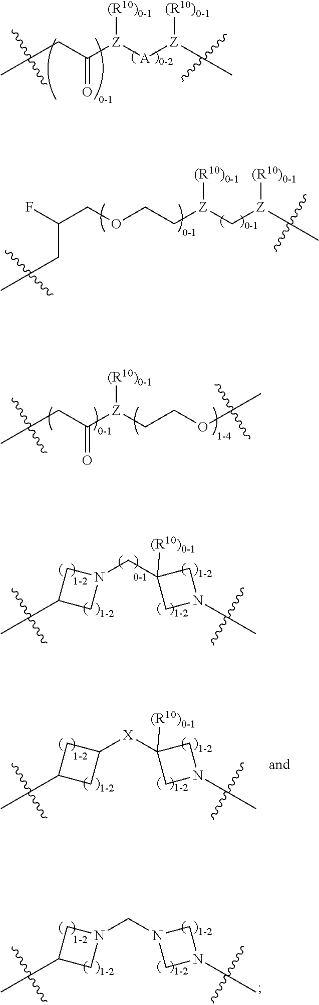

wherein:
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH;
each A is independently selected from the group consisting of CH₂, NH, and O;
X is selected from the group consisting of CH₂, NH, and O; and
Z is selected from the group consisting of CH₂, CH, C, NH, N, and O.

In another embodiment, the bifunctional compound of Formula (I) is a compound of Example 1-433, or a pharmaceutically acceptable salt, solvate, enantiomer, stereoisomer, or isotopic derivative thereof. In yet another embodiment, the bifunctional compound of Formula (I) is a compound of Example 1-433, or a pharmaceutically acceptable salt thereof.

### Pharmaceutical Compositions

The present further includes pharmaceutical compositions comprising a therapeutically effective amount of a bifunctional compound of Formula (I).

In one embodiment, a bifunctional compound of Formula (I) is formulated for administration to a patient in need thereof. In one embodiment, a bifunctional compound of Formula (I) is formulated for oral administration. Illustrative modes of administration for a compound of Formula (I) includes systemic or local administration such as oral, nasal, parenteral, transdermal, subcutaneous, vaginal, buccal, rectal, ocular, or topical administration modes. In one embodiment, the compound of Formula (I), or a pharmaceutically acceptable salt or hydrate thereof, is administered orally. In one embodiment, the compound of Formula (I) is administered as a tablet, a capsule, a caplet, a solution, a suspension, a syrup, a granule, a bead, a powder, or a pellet.

For example, in one embodiment, a bifunctional compound of Formula (I) is formulated as a tablet that comprises none, one, two, or more of each of the following: an emulsifier, a surfactant, a binder, a disintegrant, a glidant, and a lubricant.

In one embodiment, the emulsifier is selected from the group consisting of Tween 80, Labrasol, HPMC, DOSS, caproyl 909, labrafac, labrafil, peceol, transcutol, capmul MCM, capmul PG-12, captex 355, gelucire, and vitamin E TGPS. In one embodiment, the emulsifier is hydroxypropylmethylcellulose (HPMC).

In one embodiment, the surfactant is selected from the group consisting of vitamin E polyethylene glycol succinate, Tween 20, Tween 80, Span 20, Span 80, sodium docusate (e.g., AOT), sodium lauryl sulfate, and poloxamers (e.g., poloxamer 407, Kolliphor® EL, Pluronic F68). In one embodiment, the surfactant is vitamin E polyethylene glycol succinate.

In one embodiment, the binder (also referred to herein as a filler) is selected from the group consisting of microcrystalline cellulose, lactose monohydrate, sucrose, glucose, and sorbitol.

In one embodiment, the disintegrant is selected from the group consisting of sodium starch glycolate, sodium carboxymethyl cellulose, calcium carboxymethyl cellulose, croscarmellose sodium, crospovidone, chitosan, agar, alginic acid, calcium alginate, methyl cellulose, microcrystalline cellulose, powdered cellulose, lower alkylsubstituted hydroxypropyl cellulose, hydroxylpropyl starch, low-substituted hydroxypropylcellulose, polacrilin potassium, starch, pregelatinized starch, sodium alginate, magnesium aluminum silicate, polacrilin potassium, povidone, and sodium starch glycolate. In one embodiment, the disintegrant is croscarmellose sodium.

The glidant refers to a substance used to promote powder flow by reducing interparticle cohesion. In one embodiment, in a dosage form of the disclosure, the glidant is selected from the group consisting of silicon dioxide, silica colloidal anhydrous, starch, and talc.

The lubricant refers to a substance that prevents ingredients from sticking and/or clumping together in the machines used in preparation of the dosage forms of the disclosure. In one embodiment, in a dosage form of the disclosure, the lubricant is selected from the group consisting of magnesium stearate, sodium stearyl fumarate, stearic acid, and vegetable stearin.

The pharmaceutical compositions containing a bifunctional compound of Formula (I) may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of a compound of Formula (I) into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, using a coating such as lecithin, by the maintenance of the required particle size in the case of dispersion and/or by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, a controlled release polymer or other material such as aluminum monostearate or gelatin.

Sterile injectable solutions can be prepared by incorporating a compound of Formula (I) in the required amount in an appropriate solvent with one or a combination of ingredients enumerated above, as required, followed by filtered sterilization. Generally, dispersions are prepared by incorporating the active agent or compound into a sterile vehicle that contains a basic dispersion medium and the required other ingredients from those enumerated above. In the case of sterile powders for the preparation of sterile injectable solutions, methods of preparation are vacuum drying and freeze-drying that yield a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For oral therapeutic administration, a bifunctional compound of Formula (I) can be incorporated with excipients and used in the form of tablets, troches, or capsules. Oral compositions can also be prepared using a fluid carrier, wherein the agent or compound is administered by the oral route. Pharmaceutically compatible binding agents, and/or adjuvant materials can be included as part of the composition. The tablets, pills, capsules, troches and the like can contain any of the following ingredients, or materials of a similar nature: a binder such as microcrystalline cellulose, gum tragacanth or gelatin; an excipient such as starch or lactose; a disintegrating agent such as alginic acid, sodium starch glycolate (Primojel®), or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the agents or compounds may be delivered in the form of an aerosol spray from pressured container or dispenser, which may contain a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants may include, for example, for transmucosal administration, detergents, bile salts, and fusidic acid derivatives. Transmucosal administration can be accomplished using nasal sprays, suppositories or sublingual or buccal formulations. For transdermal administration, the active compounds may be formulated into ointments, salves, gels, creams, or transdermal patches.

In one aspect, a bifunctional compound of Formula (I) is prepared with pharmaceutically acceptable carriers that will protect the compound against rapid elimination from the body, such as a controlled release formulation, an implant, or a microencapsulated delivery system. Biodegradable, biocompatible polymers can be used, such as ethylene vinyl acetate, polyanhydrides, polyglycolic acid, collagen, polyorthoesters, and polylactic acid to prepare controlled release or sustained release formulations. Methods for preparation of such formulations will be apparent to those skilled in the art.

Liposomal suspensions (including liposomes targeted to infected cells with monoclonal antibodies to viral antigens) can be used as pharmaceutically acceptable carriers. These can be prepared according to methods known in the art, for example, as described in U.S. Pat. No. 4,522,811.

It is especially advantageous to formulate oral or parenteral compositions in unit dosage form for ease of administration, uniformity of dosage, and compliance. Unit dosage form as used herein refers to physically discrete units suited as unitary dosages for the subject to be treated; each unit containing a predetermined quantity of active compound determined to produce the desired therapeutic effect in association with the required pharmaceutical carrier. The unit dosage forms of an application are dictated by and directly dependent on several factors, such as, for example, the unique characteristics of a compound of Formula (I) the particular therapeutic effect to be achieved, and the condition of the patient being treated.

Pharmaceutical compositions disclosed herein can be included in a container, pack, or dispenser together with printed instructions for administration.

Illustrative pharmaceutical compositions are tablets and gelatin capsules comprising a bifunctional compound of Formula (I) and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant or sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the salt such as cyclodextrin, hydroxypropyl-cyclodextrin, PEG400, and/or PEG200.

For preparing pharmaceutical compositions from a bifunctional compound of Formula (I), pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar, or lactose. Tablets, powders, cachets, and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions, and emulsions, e.g., water or water-propylene glycol solutions for parenteral injection or addition of sweeteners and opacifiers for oral solutions, suspensions, and emulsions. Liquid form preparations may also include solutions for intranasal administration.

Liquid, particularly injectable, compositions can, for example, be prepared by dissolution, dispersion, etc. For example, the bifunctional compound dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

Parenteral injectable administration is generally used for subcutaneous, intramuscular, or intravenous injections and infusions. Injectables can be prepared in conventional forms, either as liquid solutions or suspensions or solid forms suitable for dissolving in liquid prior to injection.

Aerosol preparations suitable for inhalation may include solutions and solids in powder form, which may be in combination with a pharmaceutically acceptable carrier, such as an inert compressed gas, e.g., nitrogen.

Also included are solid form preparations that are intended to be converted, shortly before use, to liquid form preparations for either oral or parenteral administration. Such liquid forms include solutions, suspensions, and emulsions.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can be administered in intravenous (both bolus and infusion), intraperitoneal, subcutaneous, or intramuscular form.

Pharmaceutical compositions can be prepared according to conventional mixing, granulating, or coating methods, respectively, and the present pharmaceutical compositions can contain from about 0.1% to about 99%, from about 5% to about 90%, or from about 1% to about 20% of the bifunctional compound by weight or volume.

All amounts of any component of an oral dosage form described herein, e.g., a tablet, that are indicated based on % w/w refer to the total weight of the oral dosage form, unless otherwise indicated.

### Methods of Ubiquitinating/Degrading a Target Protein in a Cell

The present invention further provides a method of ubiquitinating/degrading a target protein, e.g., IRAK-4 in a cell. The method comprises administering a bifunctional composition comprising an E3 ubiquitin ligase binding moiety and an IRAK-4 protein targeting moiety, preferably linked through a linker moiety, as otherwise described herein, wherein the E3 ubiquitin ligase binding moiety is coupled to the protein targeting moiety and wherein the E3 ubiquitin ligase binding moiety recognizes a ubiquitin pathway protein (e.g., an ubiquitin ligase, preferably an E3 ubiquitin ligase), and the protein targeting moiety recognizes the target protein such that the target protein will be ubiquitinated when the target protein is placed in proximity to the ubiquitin ligase, thus resulting in degradation/inhibition of the target protein and the control of protein levels. The control of protein levels afforded by the present invention provides treatment of a disease state or condition, which is modulated through the target protein by lowering the level of that protein in the cells of a patient.

In one embodiment, the present invention is directed to a method of treating a patient in need thereof for a disease state or condition modulated through the IRAK-4 protein where the degradation of that protein will produce a therapeutic effect in that patient, the method comprising administering to a patient in need thereof an effective amount of a bifunctional compound according to the present invention, optionally in combination with another bioactive agent. The disease state or condition may be a disease caused by a microbial agent or other exogenous agent such as a virus, bacteria, fungus, protozoa, or other microbe, or may be a disease state or condition, causally related to signalling cascades mediated by IRAK-4 protein, which leads to a disease state and/or condition.

### Methods of Treatment

In one aspect, this application pertains to methods of treating or ameliorating a disease state or condition which is modulated through or causally related to the target protein, i.e., IRAK-4.

The terms “treat”, “treating”, and “treatment”, etc., as used herein, refer to any action providing a benefit to a patient for which the present compounds may be administered, including the treatment of any disease state or condition which is modulated through the target protein to which the present compounds bind. Disease states or conditions, including cancer, inflammatory diseases/disorders, autoimmune diseases/disorders, neurodegenerative diseases, and/or cardiovascular diseases/disorders, which may be treated using compounds according to the present disclosure are set forth hereinabove.

The description provides therapeutic compositions as described herein for effectuating the degradation of the target protein for the treatment or amelioration of a disease, e.g., cancer, inflammatory diseases/disorders, autoimmune diseases/disorders, neurodegenerative diseases, and/or cardiovascular diseases/disorders. In certain additional embodiments, the disease is cancer or an inflammation disorder. In certain embodiments, the disease or condition is rheumatoid arthritis, psoriasis, lupus, or atopic dermatitis. Without wishing to be bound by theory of the proposed mechanism of action, in another aspect, the description provides a method of ubiquitinating/degrading a target protein in a cell. In certain embodiments, the method comprises administering a bifunctional compound as described herein comprising, a CLM and a ITM, preferably linked through a linker moiety, as otherwise described herein, wherein the CLM recognizes a ubiquitin pathway protein (e.g., an ubiquitin ligase), such as an cereblon E3 ubiquitin ligase and the ITM recognizes the target protein (i.e., IRAK-4) such that ubiquitination of the target protein will occur when the target protein is placed in proximity to the ubiquitin ligase, thus followed by degradation of the target protein via the proteasome and the control or reduction of target protein levels. The control of target protein levels afforded by the present disclosure provides treatment of a disease state or condition by lowering the level of the target protein in the cell, e.g., cell of a patient. In certain embodiments, the method comprises administering an effective amount of a compound as described herein, optionally including a pharmaceutically acceptable excipient.

In additional embodiments, the description provides methods for treating or ameliorating a disease, disorder or symptom thereof in a subject or a patient, e.g., an animal such as a human, comprising administering to the subject or patient in need thereof a pharmaceutical composition comprising an effective amount, e.g., a therapeutically effective amount, of a bifunctional compound of Formula (I) as described herein, or a pharmaceutically acceptable salt, solvate, or isotopic derivative thereof, and a pharmaceutically acceptable excipient, carrier, adjuvant, another bioactive agent or combination thereof, wherein the composition is effective for treating or ameliorating the disease or disorder or symptom thereof in the subject or patient.

In another embodiment, the present disclosure is directed to a method of treating a human subject or patient in need thereof for a disease state or condition modulated through the target protein where the degradation of that protein will produce a therapeutic effect in that patient, the method comprising administering to the subject or patient in need thereof an effective amount of a compound according to the present disclosure, optionally in combination with another bioactive agent. The disease state or condition may be a disease caused by a microbial agent or other exogenous agent such as a virus, bacteria, fungus, protozoa, or other microbe or may be a disease state or condition caused by expression or overexpression of the target protein, or by signaling cascades mediated by the target protein.

The term “disease state or condition” is used to describe any disease state or condition wherein protein dysregulation (i.e., the amount of protein expressed in a patient is elevated) occurs and where degradation of the protein in a subject or patient may provide beneficial therapy or relief of symptoms to a subject or patient in need thereof. In certain instances, the disease state or condition may be cured.

Disease states of conditions that may be treated using compounds according to the present disclosure include, for example, asthma, autoimmune diseases such as multiple sclerosis, various cancers, ciliopathies, cleft palate, diabetes, cardiovascular disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Hemophilia, Klinefelter's syndrome, Neurofibromatosis, Phenylketonuria, Polycystic kidney disease, acute or chronic kidney disease, (PKD1) or 4 (PKD2) Prader-Willi syndrome, Sickle-cell disease, Tay-Sachs disease, Turner syndrome, and the like.

In any aspect or embodiment described herein, the disease state or condition that may be treated using the composition and/or compound of the present disclosure may be one of: a cancer, an inflammatory disorder, an autoimmune disease, metabolic disorder, a hereditary disorder, a hormone-related disease, immunodeficiency disorder, a condition associated with cell death, a destructive bone disorder, thrombin-induced platelet aggregation, liver disease, or a cardiovascular disorder.

In any aspect or embodiment described herein, the disease state or conditions that may be treated using a compound according to the present disclosure includes, for example: (A) pulmonary disease or disease of the airway including, but not limited to, Adult Respiratory Disease Syndrome (ARDS), Chronic Obstructive Pulmonary Disease (COPD), pulmonary fibrosis, interstitial lung disease, asthma, chronic cough, and allergic rhinitis, (B) transplantation, (C) an autoimmune disease including, but not limited to, rheumatoid arthritis, systemic lupus erythematosus, multiple sclerosis, or diabetes (e g, type 1 diabetes mellitus), (D) cancer including, but not limited to, solid tumors, skin cancer or lymphoma, (E) cardiovascular disease including, but not limited to, stroke or atherosclerosis, (F) disease of the central nervous system including, but not limited to, neurodegenerative diseases, (G) non-CD 14 mediated sepsis, (H) osteoarthritis, (I) osteoporosis, (J) psoriasis and diseases of the skin including, but not limited to, rash and contact and atopic dermatitis, (K) inflammatory disorders; (L) inflammatory bowel disease (including, but not limited to, Crohn's disease and ulcerative colitis), (M) Behcet's syndrome, (N) ankylosing spondylitis, (O) sarcoidosis, (P) gout, (Q) ophthalmic diseases and conditions, and (R) CD14 mediated sepsis. In such patients, the inhibition and/or degradation of TRAK-4 in, e g, IL-1 responsive cells will block the transduction of the IL-1 initiated signal, thereby preventing NF-κB activation and thus providing a treatment for the disorder or disorders.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present disclosure include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitt's Lymphoma, B-cell ALL, Philadelphia chromosome positive ALL and Philadelphia chromosome positive CML.

In any aspect or embodiment described herein, the cancer may be selected from the group consisting of breast cancer, colorectal cancer, non-small cell lung cancer, ovarian, renal, sarcoma, melanoma, head & neck, hepatocellular, thyroid, multidrug-resistant leukemia, lymphoma, multiple myeloma, esophageal, large bowel, pancreatic, mesothelioma, carcinoma (e.g., adenocarcinoma, including esophageal adenocarcinoma), sarcoma (e.g., spindle cell sarcoma, liposarcoma, leiomyosarcoma, abdominal leiomyosarcoma, sclerosing epithelioid sarcoma) and melanoma (e.g., metastatic malignant melanoma).

In any aspect or embodiment described herein, the inflammatory disease/disorder is selected from the group consisting of ocular allergy, conjunctivitis, keratoconjunctivitis sicca, vernal conjunctivitis, allergic rhinitis, autoimmune hematological disorders (e.g., hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Stevens-Johnson syndrome, idiopathic sprue, autoimmune inflammatory bowel disease (e.g., ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, primary biliary cirrhosis, uveitis (anterior and posterior), Sjogren's syndrome, interstitial lung fibrosis, psoriatic arthritis, systemic juvenile idiopathic arthritis, nephritis, vasculitis, diverticulitis, interstitial cystitis, glomerulonephritis (e.g., including idiopathic nephrotic syndrome or minimal change nephropathy), chronic granulomatous disease, endometriosis, leptospirosis renal disease, glaucoma, retinal disease, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma, acute lung injury, acute respiratory distress syndrome, eosinophilia, hypersensitivities, anaphylaxis, fibrositis, gastritis, gastroenteritis, nasal sinusitis, ocular allergy, silica induced diseases, chronic obstructive pulmonary disease (COPD), cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, juvenile rheumatoid arthritis, dermatomyositis, encephalitis, endocarditis, endometritis, enteritis, enterocolitis, epicondylitis, epididymitis, fasciitis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, vasculitis, vulvitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, Cryopyrin Associated Periodic Syndrome (CAPS) and osteoarthritis.

In any aspect or embodiment described herein, the neurodegenerative and/or neuroinflammatory disease may be selected from the group consisting of Alzheimer's disease, Parkinson's disease, dementia, multiple sclerosis, autoimmune encephalitis, amyotrophic lateral sclerosis, Huntington's disease, cerebral ischemia, and neurodegenerative disease caused by traumatic injury, glutamate neurotoxicity, hypoxia, epilepsy and graft versus host disease.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Abbreviations

AcOH Acetic acid
Ac₂O Acetic anhydride
AIBN Azobisisobutyronitrile
CCL₄ Carbon tetrachloride
CDCl₃ Chloroform-d
CDI Carbonyldiimidazole
Cs₂CO₃ Cesium carbonate
DAST Diethylaminosulfur trifluoride
DCE Dichloroethane
DCM Dichloromethane
DIEA or DIPEA Diisopropylethylamine
DMAP Dimethylaminopyridine
DMF N,N-Dimethylformamide
DMP Dess-Martin periodinane
DMSO Dimethyl sulfoxide
DMSO-d₆ Deuterated dimethyl sulfoxide (C₂D₆SO)
EDCI 1-Ethyl-3-(3-dimethylaminopropyl)carbodiimide
ESI Electrospray ionization
EtOH Ethanol
h or hr. hours
HATU 1-[Bis(dimethylamino)methylene]-1H-1,2,3-triazolo[4,5-b]pyridinium 3-oxide hexafluorophosphate
HCl Hydrochloric acid
HNO₃ Nitric acid
HPLC High performance liquid chromatography
H₂SO₄ Sulfuric acid
i-PrOH Iso-propanol
K₂CO₃ Potassium carbonate
K₃PO₄ Potassium phosphate
LCMS Liquid chromatography-mass spectrometry
m-CPBA Meta-Chloroperbenzoic acid
MeCN Acetonitrile
MHz Megahertz
MeOH Methanol
NaBH(OAc)₃ Sodium triacetoxyborohydride
NaN₃ Sodium azide
NH₄Cl Ammonium chloride
NMR Nuclear magnetic resonance
NBS N-bromosuccinimide
Pd/C Palladium on carbon
Pd₂(dba)₃ Tris(dibenzylideneacetone)dipalladium(0)
Pd(dppf)Cl₂ CH₂Cl₂ [1,1-Bis(diphenylphosphino)ferrocene]dichloropalladium(II), complex with dichloromethane
Pd(OAc)₂ Palladium (II) acetate
PMB para-Methoxybenzyl
PPh₃ Triphenylphosphine
Py Pyridine
SFC Supercritical fluid chromatography
TBAF tetra-n-Butylammonium fluoride
t-BuOK Potassium tert-butoxide
tBuXPhos Pd G1 Chloro[2-(di-tert-butylphosphino)-2′,4′,6′-triisopropyl-1,1′-biphenyl][2-(2-aminoethyl)phenyl)]palladium(II)
TEA Triethylamine
TFA Trifluoroacetic acid
TFAA Trifluoroacetic anhydride
TF₂O Trifluoromethanesulfonic anhydride
THF Tetrahydrofuran
TLC Thin-layer chromatography
TMSOTf Trimethylsilyl trifluoromethanesulfonate
Xantphos 4,5-Bis(diphenylphosphino)-9,9-dimethylxanthene
Xphos Pd G3 (2-Dicyclohexylphosphino-2′,4′,6′-triisopropyl-1,1′-biphenyl)[2-(2′-amino-1,1′-biphenyl)]palladium(II) methanesulfonate

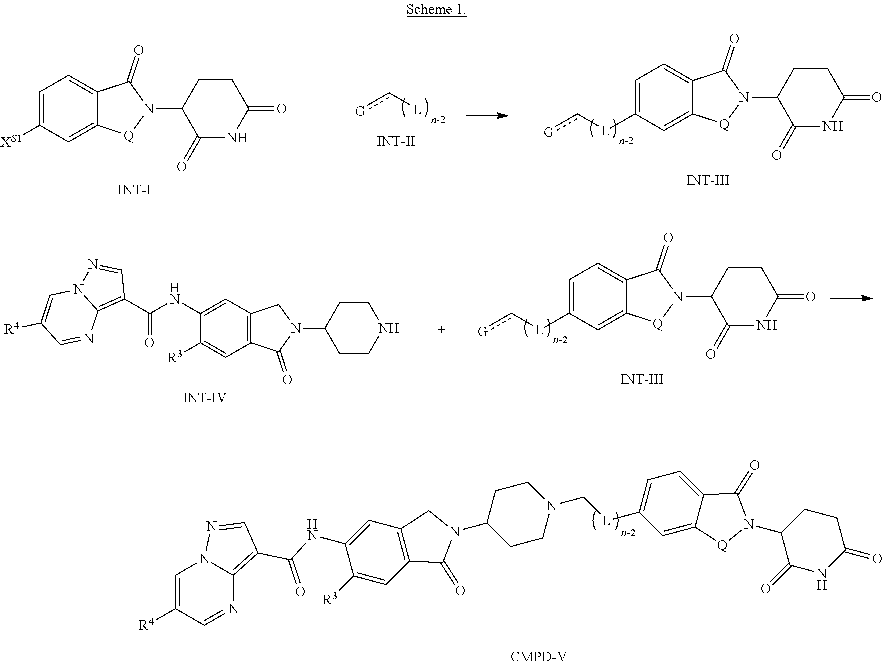

A compound of formula INT-I, wherein X^{S1} is a halide or alcohol, may be reacted with a reagent INT-II (commercially available or readily prepared using standard reaction techniques known to one skilled in the art) under etherification or alkylation conditions to produce a compound of formula INT-III, wherein L and n are as defined herein, and Q is CH₂ or C═O. Compounds of formula INT-III may react with a compound of formula INT-IV, wherein R³ is an ether, alkyl group or an amine and R⁴ is optionally H, an amine, methyl, or chloro group, through N-alkylation where G is an appropriate leaving group (e.g., OMs, OTs, Cl, etc.) or through reductive amination where G is an aldehyde or ketone to produce compound of formula CNPD-V. When G is a leaving group, suitable reaction conditions are those for an alkylation reaction, e.g., diisopropylethylamine, potassium iodide, DMSO or acetonitrile, 80° C. When G is an aldehyde, suitable reaction conditions are those for a reductive amination reaction, e.g., sodium cyanoborohydride, methanol, dichloromethane, acetic acid, room temperature.

Synthetic sequence for preparation of intermediates of generic structure INT-IV in Scheme 1.

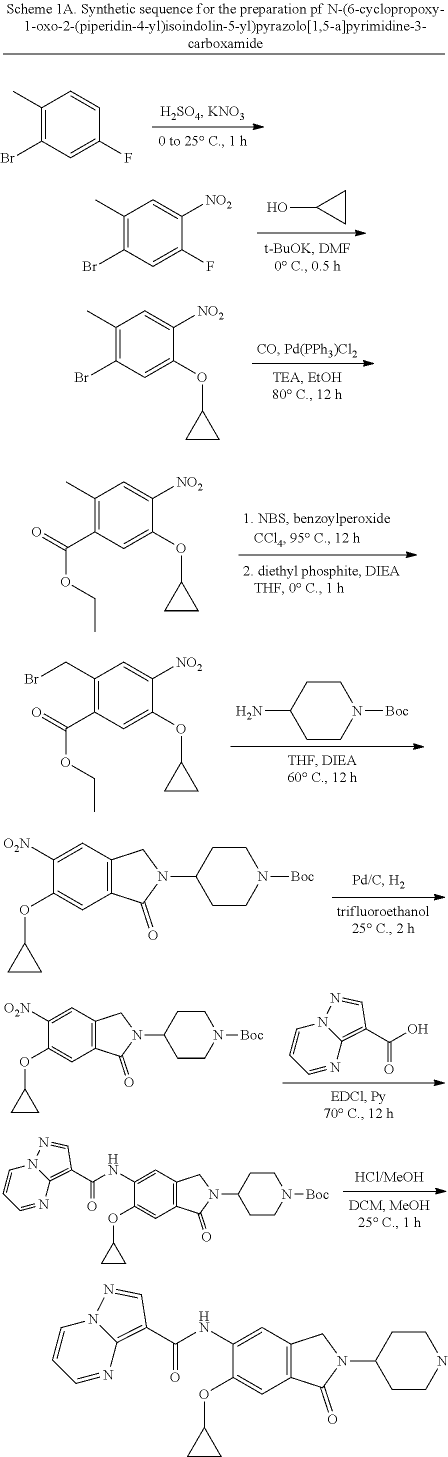

### Step 1:

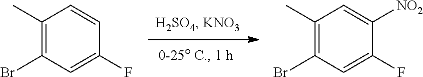

To a solution of 2-bromo-4-fluoro-1-methylbenzene (20 g, 105.81 mmol, 1 eq) in sulfuric acid (200 mL) was added potassium nitrate (10.06 g, 99.50 mmol, 0.94 eq) at 0° C. and the mixture was stirred at 25° C. for 1 h. The reaction mixture was poured into ice water (1000 mL) and extracted with ethyl acetate (300 mL×3), the organic phase was washed with a saturated solution of sodium bicarbonate (200 mL×3) and dried over anhydrous sodium sulfate, filtered, and concentrated in vacuo. The resulting residue was purified by silica gel column chromatography (petroleum ether/ethyl acetate=60:1 to 30:1) to afford 1-bromo-5-fluoro-2-methyl-4-nitrobenzene (18 g, 76.9 mmol, 73% yield) as a yellow solid. ¹H NM/IR (400 MHz, CDCl₃) δ=7.97 (d, J=7.6 Hz, 1H), 7.53 (d, J=10.0 Hz, 1H), 2.46 (s, 3H).

### Step 2:

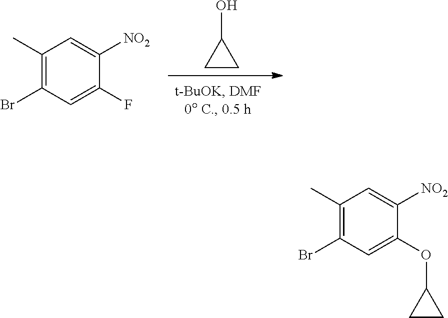

To a solution of 1-bromo-5-fluoro-2-methyl-4-nitrobenzene (17 g, 72.6 mmol, 1 eq) and cyclopropanol (5.60 g, 96.48 mmol, 1.33 eq) in N,N-dimethylformamide (170 mL) was slowly added potassium tert-butoxide in tetrahydrofuran (1 M, 88 mL, 1.21 eq) at 0° C. The reaction mixture was stirred at 0° C. for 0.5 h. The reaction mixture was slowly poured into ice water (1000 mL) and extracted with ethyl acetate (400 mL×3), the combined organic phase was washed with brine (200 mL) and dried over anhydrous sodium sulfate, filtered, and under reduced pressure. The residue was purified by silica gel column chromatography (petroleum ether/ethyl acetate=100:1 to 50:1) to afford 1-bromo-5-cyclopropoxy-2-methyl-4-nitrobenzene (17 g, 62.5 mmol, 86% yield) as a yellow solid. ¹H NMR (400 MHz, CDCl₃) δ=7.74 (s, 1H), 7.65 (s, 1H), 3.91-3.81 (m, 1H), 2.39 (s, 3H), 0.92-0.87 (m, 4H).

### Step 3:

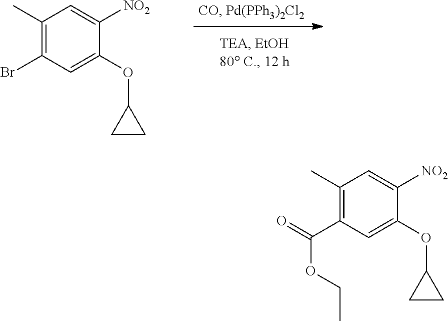 [SMILES: Cc1cc([N+](=O)[O-])c(OC2CC2)cc1Br ; CCOC(=O)c1cc(OC2CC2)c([N+](=O)[O-])cc1C]

To a solution of 1-bromo-5-cyclopropoxy-2-methyl-4-nitrobenzene (8 g, 29.4 mmol, 1 eq) in ethyl alcohol (160 mL) was added triethylamine (58.16 g, 574.8 mmol, 80.00 mL, 19.55 eq), bis(triphenylphosphine)palladium(II)dichloride (2.40 g, 3.42 mmol, 1.16e-1 eq) under an atmosphere of nitrogen. The suspension was degassed and purged with carbon monoxide in 3 cycles. The mixture was stirred under carbon monoxide (50 psi) at 80° C. for 12 h. The reaction mixture was concentrated under reduced pressure to give a residue. The residue was purified by silica gel column chromatography (petroleum ether/ethyl acetate=100:1 to 50:1) to afford ethyl 5-cyclopropoxy-2-methyl-4-nitrobenzoate (4.7 g, 17.7 mmol, 60% yield) as a yellow solid.

### Step 4:

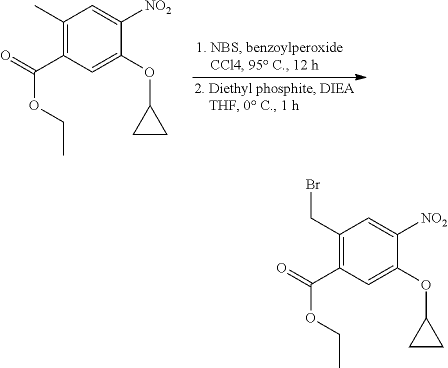 [SMILES: CCOC(=O)c1cc(OC2CC2)c([N+](=O)[O-])cc1C ; CCOC(=O)c1cc(OC2CC2)c([N+](=O)[O-])cc1CBr]

To a solution of ethyl 5-cyclopropoxy-2-methyl-4-nitrobenzoate (4.7 g, 17.7 mmol, 1 eq) in carbon tetrachloride (100 mL) was added N-bromosuccinimide (6.31 g, 35.44 mmol, 2 eq) and benzoic peroxyanhydride (860 mg, 3.55 mmol, 3 portions of 0.2 eq each). The mixture was stirred under an atmosphere of nitrogen at 95° C. for 12 h. The reaction mixture was concentrated under reduced pressure to give a residue. The residue was purified by silica gel column chromatography (petroleum ether/ethyl acetate=5:1) to afford ethyl 5-cyclopropoxy-2-(dibromomethyl)-4-nitrobenzoate (7 g, 16.5 mmol, 93% yield) as a yellow solid.

To a solution of ethyl 5-cyclopropoxy-2-(dibromomethyl)-4-nitrobenzoate (7 g, 16.55 mmol, 1 eq) in tetrahydrofuran (70 mL) was added diisopropylethylamine (3.60 g, 27.9 mmol, 4.86 mL, 1.69 eq) and diethyl phosphonate (3.12 g, 22.58 mmol, 2.91 mL, 1.36 eq). The mixture was stirred at 0° C. for 1 h. The reaction mixture was concentrated under reduced pressure to give a residue. The residue was purified by silica gel column chromatography (petroleum ether/ethyl acetate=20:1 to 6:1) to afford ethyl 2-(bromomethyl)-5-cyclopropoxy-4-nitrobenzoate (5 g, 14.5 mmol, 88% yield) as a white solid. ¹H NMR (400 MHz, CDCl₃) δ=8.01 (s, 1H), 7.89 (s, 1H), 4.87 (s, 2H), 4.48 (q, J=7.2 Hz, 2H), 4.00-3.94 (m, 1H), 1.47 (t, J=7.2 Hz, 3H), 0.95-0.89 (m, 4H).

### Step 5:

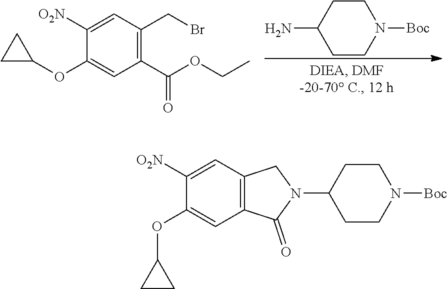 [SMILES: CCOC(=O)c1cc(OC2CC2)c([N+](=O)[O-])cc1CBr ; CC(C)(C)OC(=O)N1CCC(N)CC1 ; CC(C)(C)OC(=O)N1CCC(N2Cc3cc([N+](=O)[O-])c(OC4CC4)cc3C2=O)CC1]

To a solution of tert-butyl 4-aminopiperidine-1-carboxylate (970.6 mg, 4.85 mmol, 2.08 eq) and diisopropylethylamine (989.3 mg, 7.65 mmol, 1.33 mL, 3.29 eq) in N,N-dimethylformamide (16 mL) was added slowly dropwise a solution of ethyl 2-(bromomethyl)-5-cyclopropoxy-4-nitrobenzoate (0.8 g, 2.32 mmol, 1 eq) in N,N-dimethylformamide (8 mL) at −20° C. The mixture was then allowed to stir at 0° C. for 1 h followed by heating to 70° C. for 11 h. The reaction mixture was concentrated under reduced pressure to give a residue. The residue was purified by preparative thin layer chromatography (silicon dioxide, petroleum ether/ethyl acetate=1/1) to afford tert-butyl 4-(6-cyclopropoxy-5-nitro-1-oxoisoindolin-2-yl)piperidine-1-carboxylate (680 mg, 1.63 mmol, 70% yield) as a yellow solid. MS (ESI) m/z: 362.1 [M-t-Bu]⁺. ¹H NMR (400 MHz, CDCl₃) δ=7.90 (s, 1H), 7.84 (s, 1H), 4.49-4.39 (m, 1H), 4.35 (s, 4H), 4.01-3.93 (m, 1H), 2.95-2.82 (m, 2H), 1.90-1.82 (m, 2H), 1.76-1.65 (m, 2H), 1.49 (s, 9H), 0.95-0.86 (m, 4H).

### Step 6:

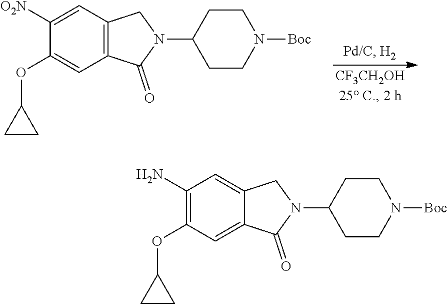 [SMILES: CC(C)(C)OC(=O)N1CCC(N2Cc3cc([N+](=O)[O-])c(OC4CC4)cc3C2=O)CC1 ; CC(C)(C)OC(=O)N1CCC(N2Cc3cc(N)c(OC4CC4)cc3C2=O)CC1]

To a solution of tert-butyl 4-(6-cyclopropoxy-5-nitro-1-oxoisoindolin-2-yl)piperidine-1-carboxylate (0.68 g, 1.63 mmol, 1 eq) in 2,2,2-trifluoroethanol (20 mL) was added palladium on carbon (200 mg, 187.9 μmol, 10% purity, 0.115 eq) under an atmosphere of nitrogen. The suspension was degassed under vacuum and purged with hydrogen for several cycles. The mixture was stirred under a balloon atmosphere of hydrogen (15 psi) at 25° C. for 2 h. The reaction mixture was filtered and the filtrate was concentrated to afford the crude product, tert-butyl 4-(5-amino-6-cyclopropoxy-1-oxoisoindolin-2-yl)piperidine-1-carboxylate (600 mg, 1.55 mmol, 95% yield) as a white solid which was used in the next step without further purification. MS (ESI) m/z: 388.2 [M+H]⁺. ¹H NMR (400 MHz, CDCl₃) δ=7.56 (s, 1H), 6.70 (s, 1H), 4.45-4.34 (m, 1H), 4.30-4.04 (m, 5H), 3.97 (q, J=9.2 Hz, 1H), 3.86-3.79 (m, 1H), 2.92-2.79 (m, 2H), 1.85-1.77 (m, 2H), 1.72-1.58 (m, 2H), 1.48 (s, 9H), 0.89-0.76 (m, 4H).

### Step 7:

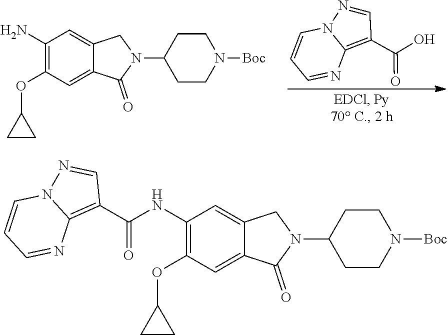

To a solution of tert-butyl 4-(5-amino-6-cyclopropoxy-1-oxoisoindolin-2-yl)piperidine-1-carboxylate (0.6 g, 1.55 mmol, 1 eq) and pyrazolo[1,5-a]pyrimidine-3-carboxylic acid (550 mg, 3.37 mmol, 2.18 eq) in pyridine (20 mL) was added 1-ethyl-3-(3-dimethylaminopropyl)carbodiimide (1.31 g, 6.82 mmol, 4.40 eq). The mixture was stirred at 70° C. for 2 h. The reaction mixture was concentrated in vacuo to give a residue. The residue was purified by silica gel column chromatography (eluted with a gradient of dichloromethane:methanol=100:1 to 10:1) to afford tert-butyl 4-(6-cyclopropoxy-1-oxo-5-(pyrazolo[1,5-a]pyrimidine-3-carboxamido)isoindolin-2-yl)piperidine-1-carboxylate (0.6 g, 1.13 mmol, 73% yield) as a yellow solid. MS (ESI) m/z: 774.3 [M+H]+. 1H NMR (400 MHz, DMSO-d6) δ=10.57 (s, 1H), 9.40 (dd, J=1.6, 7.2 Hz, 1H), 8.91 (dd, J=1.6, 4.0 Hz, 1H), 8.72 (s, 1H), 8.68 (s, 1H), 7.57 (s, 1H), 7.36 (dd, J=4.4, 7.2 Hz, 1), 4.42 (s, 2H), 4.25-4.17 (m, 2H), 4.13-4.00 (m, 2H), 2.98-2.76 (m, 2H), 1.76-1.58 (m, 4H), 1.42 (s, 9H), 0.98-0.90 (m, 2H), 0.89-0.82 (m, 2H).

### Step 8:

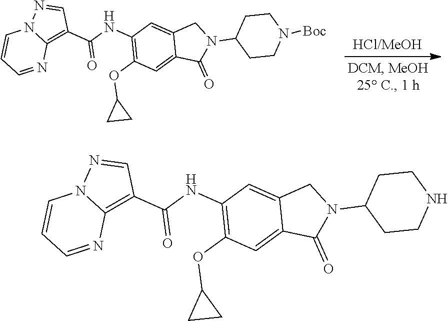 [SMILES: CC(C)(C)OC(=O)N1CCC(N2Cc3cc(NC(=O)c4cnn5cccnc45)c(OC4CC4)cc3C2=O)CC1 ; O=C(Nc1cc2c(cc1OC1CC1)C(=O)N(C1CCNCC1)C2)c1cnn2cccnc12]

To a solution of tert-butyl 4-(6-cyclopropoxy-1-oxo-5-(pyrazolo[1,5-a]pyrimidine-3-carboxamido)isoindolin-2-yl)piperidine-1-carboxylate (160 mg, 300.42 μmol, 1 eq) in methanol (5 mL) and dichloromethane (5 mL) was added a solution of hydrogen chloride in methanol (4 M, 5 mL, 66.6 eq). The mixture was stirred at 25° C. for 1 h. The reaction mixture was concentrated under reduced pressure to give a residue which was resuspended in 50 mL of dichloromethane/methanol (10:1). The pH of the solution was adjusted to 7-8 with ammonium hydroxide, dried over anhydrous sodium sulfate, filtered and concentrated under reduced pressure to afford N-(6-cyclopropoxy-1-oxo-2-(piperidin-4-yl)isoindolin-5-yl)pyrazolo[1,5-a]pyrimidine-3-carboxamide (120 mg, 277.5 mol, 92% yield) as a yellow solid which was used in the next step without further purification. MS (ESI) m/z: 433.1 [M+H]⁺.

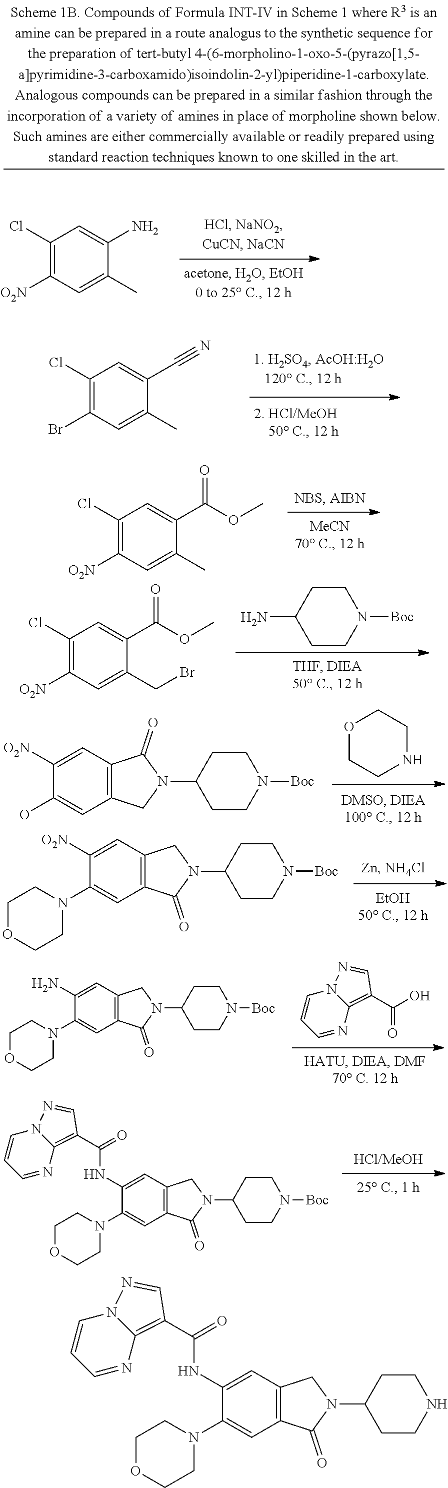

### Step 1:

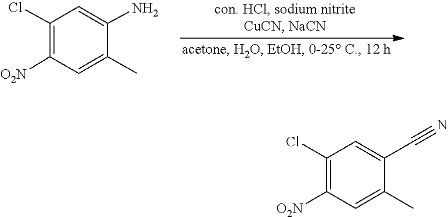

To a stirred of 5-chloro-2-methyl-4-nitro-aniline (10 g, 53.59 mmol, 1 eq) in acetone (35 mL) and water (38 mL) at 0° C. was added hydrogen chloride (12M, 11.20 mL, 2.51 eq). A solution of sodium nitrite (4.50 g, 65.22 mmol, 1.22 eq) in water (15 mL) was added dropwise and the mixture was stirred at 0° C. for 30 min. The mixture was then added dropwise to a mixture of cuprous cyanide (4.80 g, 53.6 mmol, 11.71 mL, 1 eq) and sodium cyanide (2.7 g, 55.1 mmol, 1.03 eq) in water (50 mL) and ethyl acetate (25 mL), the mixture was stirred at 25° C. for 11.5 h. The mixture was diluted with 25 mL of water, and 30 mL of ethyl acetate and extracted with ethyl acetate (10 mL×3). The combined organic layers were washed with a 2M aqueous solution of sodium hydroxide (10 mL×3) and brine (10 mL×3), dried over anhydrous sodium sulfate, filtered, and concentrated under reduced pressure to give a residue. The residue was purified by column chromatography (silicon dioxide, petroleum ether/ethyl acetate=40/1 to 5/1) to afford 5-chloro-2-methyl-4-nitro-benzonitrile (5.5 g, 27.98 mmol, 52% yield) as a brown solid.

### Step 2:

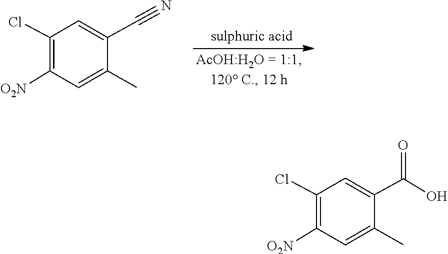

To a solution of 5-chloro-2-methyl-4-nitrobenzonitrile (5.5 g, 27.98 mmol, 1 eq) in acetic acid (55 mL) and water (55 mL) was added sulfuric acid (55 mL). The mixture was stirred at 120° C. for 12 h. To the mixture was added 200 mL of water, and the solid was removed by filtration and dried to afford 5-chloro-2-methyl-4-nitrobenzoic acid (5.5 g, 25.51 mmol, 91% yield) as a gray solid.

### Step 3:

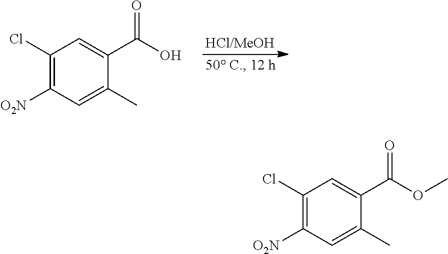

To a solution of 5-chloro-2-methyl-4-nitro-benzoic acid (5.5 g, 25.51 mmol, 1 eq) in hydrogen chloride/methanol (55 mL). The mixture was stirred at 50° C. for 12 h. The mixture was concentrated in vacuo, then diluted by ethyl acetate (100 mL). After the addition of 50 mL of water, the mixture was extracted with ethyl acetate (10 mL×3). The combined organic layers were dried over anhydrous sodium sulfate, filtered, and concentrated under reduced pressure to obtain methyl 5-chloro-2-methyl-4-nitrobenzoate (5 g, 21.8 mmol, 85% yield) as a brown solid.

### Step 4:

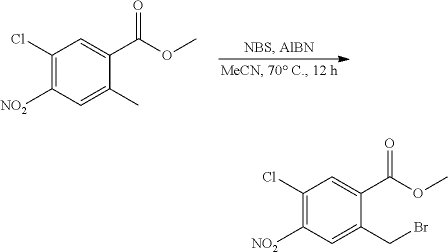 [SMILES: COC(=O)c1cc(Cl)c([N+](=O)[O-])cc1C ; COC(=O)c1cc(Cl)c([N+](=O)[O-])cc1CBr]

To a solution of methyl 5-chloro-2-methyl-4-nitro-benzoate (2 g, 8.71 mmol, 1 eq) in acetonitrile (20 mL) was added NBS (3.88 g, 21.78 mmol, 2.5 eq) and azobisisobutyronitrile (214.5 mg, 1.31 mmol, 0.15 eq). The mixture was stirred at 70° C. for 12 h. The reaction mixture was filtered and concentrated under reduced pressure to give a residue. The residue was purified by column chromatography (silicon dioxide, petroleum ether/ethyl acetate=1/0 to 10/1) to afford methyl 2-(bromomethyl)-5-chloro-4-nitrobenzoate (1.9 g, 6.16 mmol, 70% yield) as a brown solid.

### Step 5:

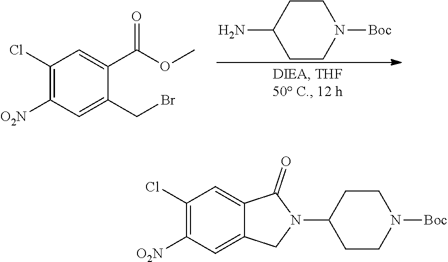 [SMILES: COC(=O)c1cc(Cl)c([N+](=O)[O-])cc1CBr ; CC(C)(C)OC(=O)N1CCC(N)CC1 ; CC(C)(C)OC(=O)N1CCC(N2Cc3cc([N+](=O)[O-])c(Cl)cc3C2=O)CC1]

To a stirred solution of methyl 2-(bromomethyl)-5-chloro-4-nitrobenzoate (3.70 g, 18.48 mmol, 3 eq) in tetrahydrofuran (10 mL) was added diisopropylethylamine (875.5 mg, 6.77 mmol, 1.18 mL, 1.1 eq), followed by dropwise addition of a solution of methyl 2-(bromomethyl)-5-chloro-4-nitro-benzoate (1.9 g, 6.16 mmol, 1 eq) in tetrahydrofuran (8 mL). The mixture was stirred at 50° C. for 12 h. The reaction mixture was filtered and concentrated under reduced pressure to give a residue which was purified by column chromatography (silicon dioxide, petroleum ether/ethyl acetate=5/1 to 1/1) to afford tert-butyl 4-(6-chloro-5-nitro-1-oxoisoindolin-2-yl)piperidine-1-carboxylate (2.1 g, 5.31 mmol, 86% yield) as a yellow solid. MS (ESI) m/z: 340.2 [M-t-Bu]⁺.

### Step 6:

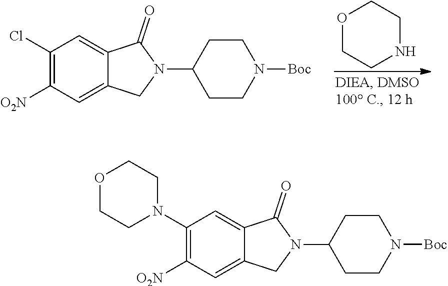 [SMILES: CC(C)(C)OC(=O)N1CCC(N2Cc3cc([N+](=O)[O-])c(Cl)cc3C2=O)CC1 ; C1COCCN1 ; CC(C)(C)OC(=O)N1CCC(N2Cc3cc([N+](=O)[O-])c(N4CCOCC4)cc3C2=O)CC1]

To a solution of tert-butyl 4-(6-chloro-5-nitro-1-oxoisoindolin-2-yl)piperidine-1-carboxylate (2.1 g, 5.31 mmol, 1 eq), morpholine (9.24 g, 106.10 mmol, 9.34 mL, 20 eq) in DMSO (10 mL) was added diisopropylethylamine (2.06 g, 15.92 mmol, 2.77 mL, 3 eq). The mixture was stirred at 100° C. for 12 h. The reaction mixture was quenched by the addition of water (100 mL) at 0° C., and then diluted with ethyl acetate (300 mL) and extracted with ethyl acetate (100 mL×3). The combined organic layers were washed with brine (100 mL×3), dried over anhydrous sodium sulfate, filtered, and concentrated under reduced pressure to give a residue. The residue was purified by column chromatography (silicon dioxide, petroleum ether/ethyl acetate=5/1 to 0/1) to afford tert-butyl 4-(6-morpholino-5-nitro-1-oxoisoindolin-2-yl)piperidine-1-carboxylate (2.1 g, 4.70 mmol, 88% yield) as a yellow solid. MS (ESI) m/z: 447.4 [M+H]⁺.

### Step 7:

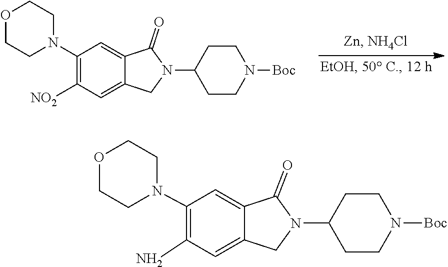 [SMILES: CC(C)(C)OC(=O)N1CCC(N2Cc3cc([N+](=O)[O-])c(N4CCOCC4)cc3C2=O)CC1 ; CC(C)(C)OC(=O)N1CCC(N2Cc3cc(N)c(N4CCOCC4)cc3C2=O)CC1]

To a solution of tert-butyl 4-(6-morpholino-5-nitro-1-oxoisoindolin-2-yl)piperidine-1-carboxylate (2.1 g, 4.70 mmol, 1 eq) in ethanol (50 mL) was added ammonium chloride (3.77 g, 70.55 mmol, 15 eq) and zinc powder (4.61 g, 70.55 mmol, 15 eq). The mixture was stirred at 50° C. for 12 h. The reaction mixture was filtered and concentrated under reduced pressure to give a residue which was resuspended in ethyl acetate (30 mL) and water (30 mL). The reaction mixture was extracted with ethyl acetate (10 mL×3). The combined organic layers were washed with brine (10 mL×3), dried over anhydrous sodium sulfate, filtered, and concentrated under reduced pressure to give a residue. The residue was purified by column chromatography (silicon dioxide, petroleum ether/ethyl acetate=3/1 to 1/5) to afford tert-butyl 4-(5-amino-6-morpholino-1-oxoisoindolin-2-yl)piperidine-1-carboxylate (1.9 g, 4.56 mmol, 96% yield) as a white solid. MS (ESI) m/z: 417.4 [M+H]⁺.

### Step 8:

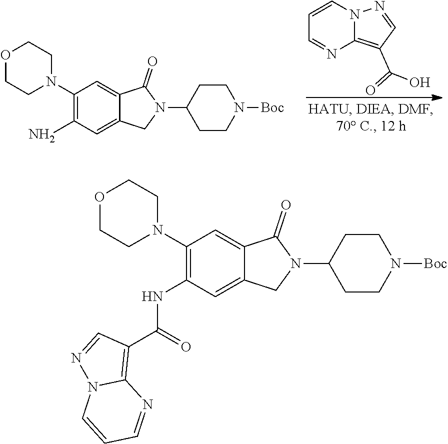 [SMILES: CC(C)(C)OC(=O)N1CCC(N2Cc3cc(N)c(N4CCOCC4)cc3C2=O)CC1 ; O=C(O)c1cnn2cccnc12 ; CC(C)(C)OC(=O)N1CCC(N2Cc3cc(NC(=O)c4cnn5cccnc45)c(N4CCOCC4)cc3C2=O)CC1]

To a solution of tert-butyl 4-(5-amino-6-morpholino-1-oxoisoindolin-2-yl)piperidine-1-carboxylate (1 g, 2.40 mmol, 1 eq) and pyrazolo[1,5-a]pyrimidine-3-carboxylic acid (587.5 mg, 3.60 mmol, 1.5 eq) in DMF (20 mL), was added diisopropylethylamine (930.89 mg, 7.20 mmol, 1.25 mL, 3 eq) and HATU (1.64 g, 4.32 mmol, 1.8 eq). The mixture was stirred at 70° C. for 12 h. The reaction mixture was quenched by addition of water (100 mL) at 0° C., and then diluted with ethyl acetate (300 mL) and extracted with ethyl acetate (100 mL×3). The combined organic layers were washed with brine (100 mL×3), dried over anhydrous sodium sulfate, filtered, and concentrated under reduced pressure to give a residue which was purified by column chromatography (silicon dioxide, petroleum ether/ethyl acetate=1/4 to 0/1) to afford tert-butyl 4-(6-morpholino-1-oxo-5-(pyrazolo[1,5-a]pyrimidine-3-carboxamido)isoindolin-2-yl)piperidine-1-carboxylate (850 mg, 1.51 mmol, 63% yield) as a brown solid. MS (ESI) m/z: 562.4 [M+H]⁺.

### Step 9:

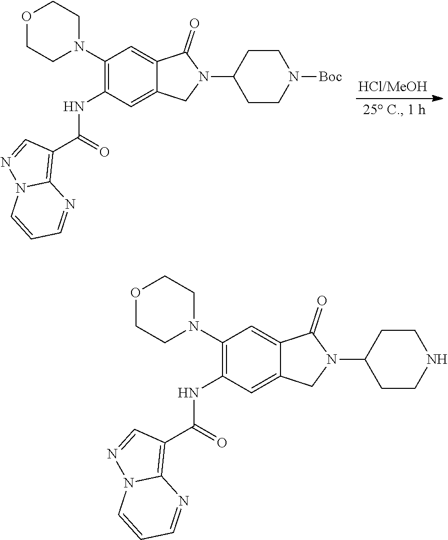

A solution of tert-butyl 4-(6-morpholino-1-oxo-5-(pyrazolo[1,5-a]pyrimidine-3-carboxamido)isoindolin-2-yl)piperidine-1-carboxylate (600 mg, 1.07 mmol, 1 eq) in hydrogen chloride/methanol (5 mL) was stirred at 25° C. for 1 h. The reaction mixture was concentrated under reduced pressure to give a residue. Then to the mixture was added 2 mL of methanol and 0.04 mL of ammonium hydroxide, the mixture was concentrated in vacuo, and suspended in 5 mL of THF. The reaction mixture was filtered and concentrated under reduced pressure to yield N-(6-morpholino-1-oxo-2-(piperidin-4-yl)isoindolin-5-yl)pyrazolo[1,5-a]pyrimidine-3-carboxamide (500 mg, crude) as a white solid. MS (ESI) m/z: 462.2 [M+H]⁺.

Alternatively, the intermediate afforded after step 5 of Scheme 1B could be transformed under Suzuki-Miyaura coupling conditions as exemplified in the preparation of tert-butyl 4-(6-cyclopropyl-5-nitro-1-oxoisoindolin-2-yl)piperidine-1-carboxylate.

### Step 1:

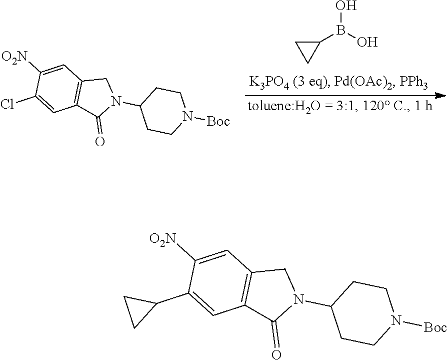 [SMILES: CC(C)(C)OC(=O)N1CCC(N2Cc3cc([N+](=O)[O-])c(Cl)cc3C2=O)CC1 ; OB(O)C1CC1 ; CC(C)(C)OC(=O)N1CCC(N2Cc3cc([N+](=O)[O-])c(C4CC4)cc3C2=O)CC1]

To a solution of tert-butyl 4-(6-chloro-5-nitro-1-oxo-isoindolin-2-yl)piperidine-1-carboxylate (1 g, 2.53 mmol, 1 eq) and cyclopropylboronic acid (868.01 mg, 10.11 mmol, 4 eq) in toluene (3 mL) and water (1 mL) was added potassium phosphate (3.22 g, 15.16 mmol, 6 eq) and palladium acetate (283.59 mg, 1.26 mmol, 0.5 eq), and triphenylphosphine (331.31 mg, 1.26 mmol, 0.5 eq). The mixture was stirred at 120° C. for 1 h. The reaction mixture was concentrated under reduced pressure and the resulting residue was diluted with ethyl acetate (50 mL) and extracted with ethyl acetate (50 mL×3). The combined organic layers were washed with brine (50 mL×3), dried over sodium sulfate, filtered, and concentrated under reduced pressure to give a residue. The residue was purified by column chromatography (SiO₂, eluted with a gradient from 20 to 50% ethyl acetate in petroleum ether) to afford tert-butyl 4-(6-cyclopropyl-5-nitro-1-oxo-isoindolin-2-yl)piperidine-1-carboxylate (0.7 g, 1.74 mmol, 69% yield) as a white solid. MS (ESI) m/z: 345.9 [M-t-Bu]⁺.

### Step 2:

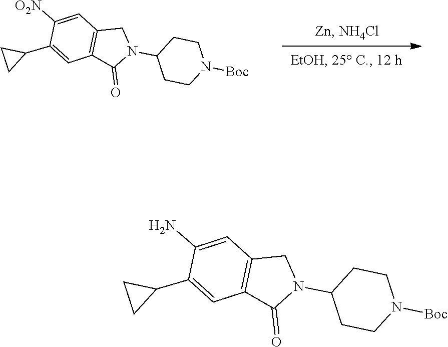 [SMILES: CC(C)(C)OC(=O)N1CCC(N2Cc3cc([N+](=O)[O-])c(C4CC4)cc3C2=O)CC1 ; CC(C)(C)OC(=O)N1CCC(N2Cc3cc(N)c(C4CC4)cc3C2=O)CC1]

To a solution of tert-butyl 4-(6-cyclopropyl-5-nitro-1-oxo-isoindolin-2-yl)piperidine-1-carboxylate (0.6 g, 1.49 mmol, 1 eq) in ethanol (10 mL) was added zinc (977.3 mg, 14.95 mmol, eq) and ammonium chloride (799.46 mg, 14.95 mmol, 10 eq). The mixture was stirred at 25° C. for 1 h. The reaction mixture was filtered and the solid was washed with ethyl acetate (50 mL×2), the filtrate was concentrated under reduced pressure to give a residue. The residue was purified by column chromatography (SiO₂, eluted with a gradient from 10 to 50% ethyl acetate in petroleum ether) to afford tert-butyl 4-(5-amino-6-cyclopropyl-1-oxo-isoindolin-2-yl)piperidine-1-carboxylate (0.5 g, 1.35 mmol, 90% yield) as a white solid. MS (ESI) m/z: 316.4 [M-t-Bu]⁺.

### Step 3:

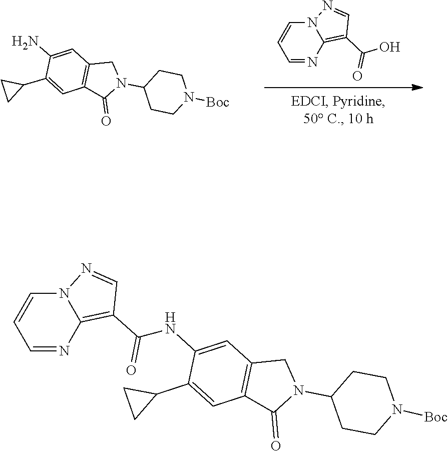 [SMILES: CC(C)(C)OC(=O)N1CCC(N2Cc3cc(N)c(C4CC4)cc3C2=O)CC1 ; O=C(O)c1cnn2cccnc12 ; CC(C)(C)OC(=O)N1CCC(N2Cc3cc(NC(=O)c4cnn5cccnc45)c(C4CC4)cc3C2=O)CC1]

To a solution of tert-butyl 4-(5-amino-6-cyclopropyl-1-oxo-isoindolin-2-yl)piperidine-1-carboxylate (0.45 g, 1.21 mmol, 1 eq) and pyrazolo[1,5-a]pyrimidine-3-carboxylic acid (296.43 mg, 1.82 mmol, 1.5 eq) in pyridine (10 mL) was added 1-ethyl-3(3-dimethylpropylamine) carbodiimide (696.68 mg, 3.63 mmol, 3 eq). The mixture was stirred at 50° C. for 1 h. The reaction mixture was concentrated under reduced pressure to give a residue. The residue was purified by column chromatography (silica gel, dichloromethane:methanol=30/1 to 10/1) to afford tert-butyl 4-[6-cyclopropyl-1-oxo-5-(pyrazolo[1,5-a]pyrimidine-3-carbonylamino)isoindolin-2-yl]piperidine-1-carboxylate (0.5 g, 967.9 μmol, 80% yield) as a white solid. MS (ESI) m/z: 517.1 [M+H]⁺.

### Step 4:

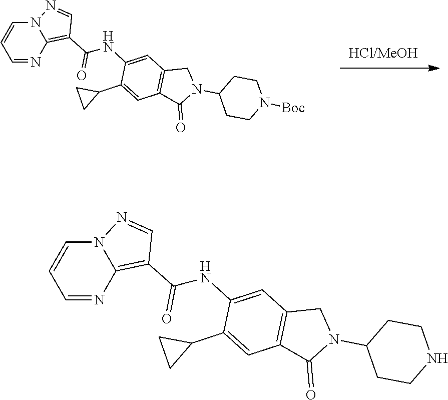 [SMILES: CC(C)(C)OC(=O)N1CCC(N2Cc3cc(NC(=O)c4cnn5cccnc45)c(C4CC4)cc3C2=O)CC1 ; O=C(Nc1cc2c(cc1C1CC1)C(=O)N(C1CCNCC1)C2)c1cnn2cccnc12]

To a solution of tert-butyl 4-[6-cyclopropyl-1-oxo-5-(pyrazolo[1,5-a]pyrimidine-3-carbonylamino)isoindolin-2-yl]piperidine-1-carboxylate (0.4 g, 774.3 μmol, 1 eq) in DCM (5 mL) was added a solution of hydrochloric acid in methanol (20 mL). The mixture was stirred at 25° C. for 1 h. The reaction mixture was concentrated under reduced pressure to provide N-[6-cyclopropyl-1-oxo-2-(4-piperidyl)isoindolin-5-yl]pyrazolo[1,5-a]pyrimidine-3-carboxamide (0.3 g, 720.33 μmol, 93% yield) as a white solid which was used without further purification. MS (ESI) m/z: 417.0 [M+H]⁺.

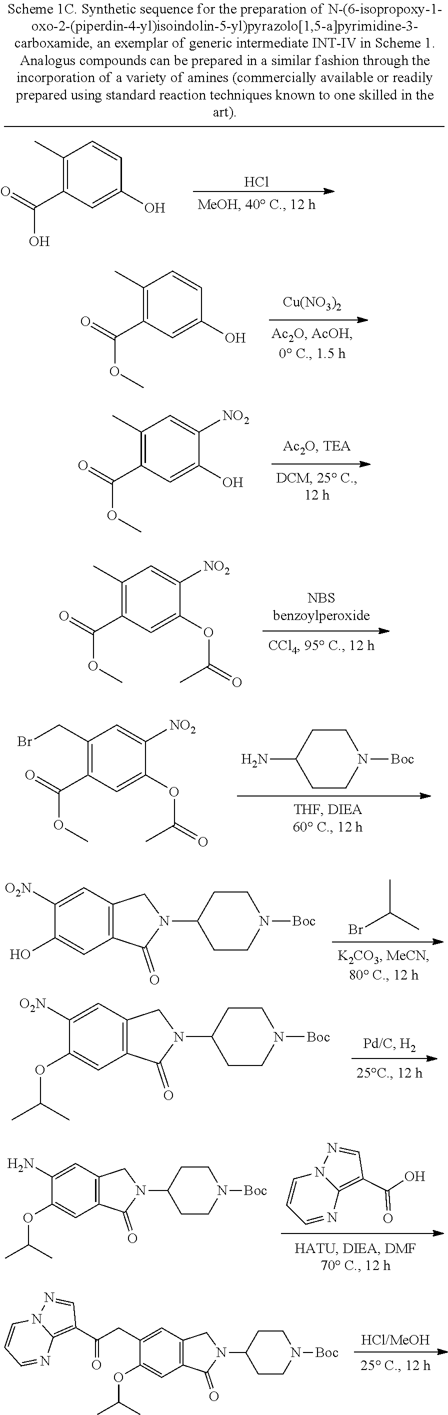

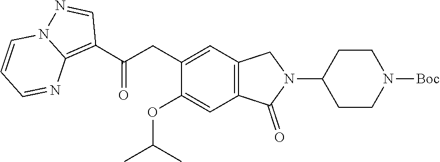 [SMILES: CC(C)Oc1cc2c(cc1CC(=O)c1cnn3cccnc13)CN(C1CCN(C(=O)OC(C)(C)C)CC1)C2=O]

### Step 1:

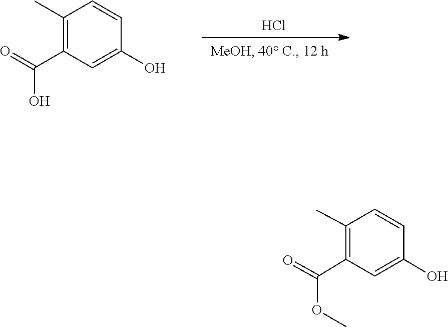

5-hydroxy-2-methylbenzoic acid (5.0 g, 32.9 mmol, 1 eq) and hydrogen chloride/methanol (4 M, 40 mL, 4.87 eq) were stirred at 40° C. for 12 h. The reaction mixture was then concentrated under reduced pressure to afford methyl 5-hydroxy-2-methylbenzoate (5.0 g, 30.1 mmol, 92% yield) as a yellow solid which was used in the next step without further purification. MS (ESI) m/z: 167.2 [M+H]+.

### Step 2:

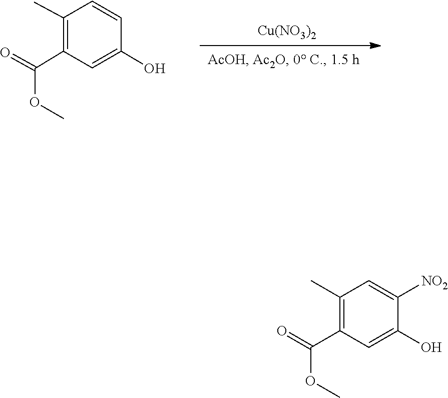

To an ice-cooled solution of methyl 5-hydroxy-2-methylbenzoate (8.0 g, 48.1 mmol, 1 eq) in HOAc (30 mL) and acetic anhydride (60 mL) was added copper (II) nitrate, trihydrate (17.60 g, 72.85 mmol, 1.51 eq). The reaction mixture was allowed to stir at 0° C. for 1.5 h. The reaction mixture was poured into ice water (1000 mL) and extracted with ethyl acetate (400 mL×3). To the combined organic phase was added a saturated aqueous solution of sodium bicarbonate (600 mL) and the mixture was stirred at 25° C. for 12 h. The organic phase was then separated, and the aqueous phase was extracted with ethyl acetate (200 mL×2). The combined organic phase was washed with water and dried over anhydrous sodium sulfate, filtered, and concentrated under reduced pressure to give a residue. The residue was purified by column chromatography (silicon dioxide, eluted with petroleum ether/ethyl acetate=20:1 to 10:1) to afford methyl 5-hydroxy-2-methyl-4-nitrobenzoate (1.3 g, 6.16 mmol, 13% yield) as a yellow solid.

### Step 3:

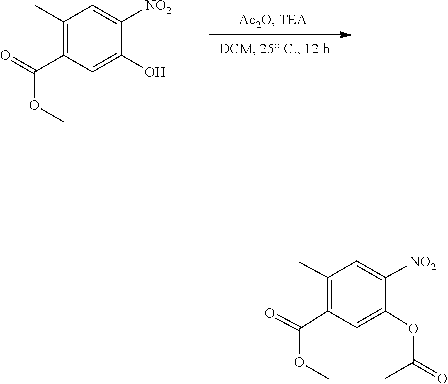 [SMILES: COC(=O)c1cc(O)c([N+](=O)[O-])cc1C ; COC(=O)c1cc(OC(C)=O)c([N+](=O)[O-])cc1C]

To a solution of methyl 5-hydroxy-2-methyl-4-nitrobenzoate (2.5 g, 11.8 mmol, 1 eq) in dichloromethane (15 mL) was added triethylamine (4.79 g, 47.3 mmol, 6.59 mL, 4 eq) and acetic anhydride (3.02 g, 29.6 mmol, 2.77 mL, 2.5 eq). The mixture was stirred at 25° C. for 12 h. The reaction mixture was poured into water (200 mL) and extracted with ethyl acetate (100 mL×3). The combined organic phase was washed with water (100 mL×3), dried over anhydrous sodium sulfate, filtered, and concentrated under reduced pressure to give a residue. The residue was purified by column chromatography (silicon dioxide, eluted with petroleum ether/ethyl acetate=20:1 to 10:1) to afford methyl 5-acetoxy-2-methyl-4-nitrobenzoate (2.8 g, 11.1 mmol, 93% yield) as a yellow solid.

### Step 4:

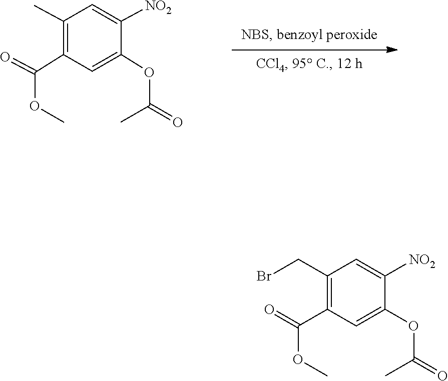 [SMILES: COC(=O)c1cc(OC(C)=O)c([N+](=O)[O-])cc1C ; COC(=O)c1cc(OC(C)=O)c([N+](=O)[O-])cc1CBr]

To a stirred solution of methyl 5-acetoxy-2-methyl-4-nitrobenzoate (2.4 g, 9.5 mmol, 1 eq) in carbon tetrachloride (40 mL) was added NBS (3.37 g, 18.96 mmol, 2 eq) and benzoyl peroxide (344.4 mg, 1.42 mmol, 0.15 eq). The mixture was stirred at 95° C. for 12 h under an atmosphere of nitrogen. The reaction mixture was concentrated under reduced pressure to give a residue. The residue was purified by column chromatography (silicon dioxide, eluted with petroleum ether/ethyl acetate=20:1 to 5:1) to afford methyl 5-acetoxy-2-(bromomethyl)-4-nitrobenzoate (3.1 g, 9.3 mmol, 98% yield) as a yellow solid.

### Step 5:

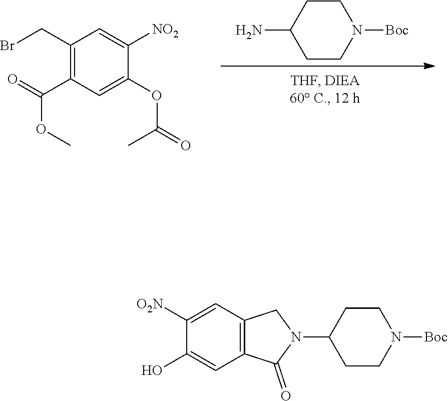 [SMILES: COC(=O)c1cc(OC(C)=O)c([N+](=O)[O-])cc1CBr ; CC(C)(C)OC(=O)N1CCC(N)CC1 ; CC(C)(C)OC(=O)N1CCC(N2Cc3cc([N+](=O)[O-])c(O)cc3C2=O)CC1]

To a solution of tert-butyl 4-aminopiperidine-1-carboxylate (7.48 g, 37.3 mmol, 4 eq) in tetrahydrofuran (20 mL) was added diisopropylethylamine (2.41 g, 18.7 mmol, 3.25 mL, 2 eq), followed by the slow addition of a solution of methyl 5-acetoxy-2-(bromomethyl)-4-nitrobenzoate (3.1 g, 9.3 mmol, 1 eq) in tetrahydrofuran (10 mL) over a period of 30 min at 40° C. The mixture was then allowed to stir at 60° C. for 12 h. The reaction mixture was concentrated under reduced pressure to give a residue. The residue was purified by preparative HPLC (column: Phenomenex Synergi Max-RP 250×50 mm×10 um; mobile phase: [water (0.225% formic acid)-acetonitrile]; B %: 30%-60%) to afford tert-butyl 4-(6-hydroxy-5-nitro-1-oxoisoindolin-2-yl)piperidine-1-carboxylate (1.0 g, 2.6 mmol, 28% yield) as a yellow solid. MS (ESI) m/z: 322.2 [M-tBu+H]⁺. ¹H NMR (400 MHz, CDCl₃) δ=10.67 (s, 1H), 8.24 (s, 1H), 7.63 (s, 1H), 4.48-4.38 (m, 1H), 4.37 (s, 2H), 4.34-4.21 (m, 2H), 2.88 (br t, J=12.0 Hz, 2H), 1.86 (br dd, J=2.0, 12.0 Hz, 2H), 1.69 (br dd, J=4.0, 12.4 Hz, 2H), 1.49 (s, 9H).

### Step 6:

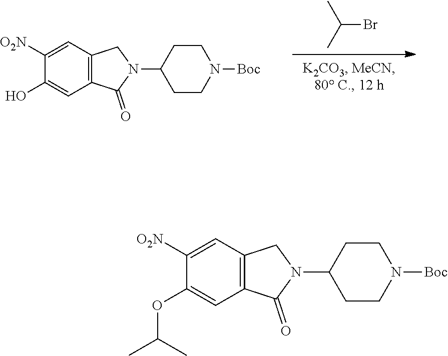

To a solution of tert-butyl 4-(6-hydroxy-5-nitro-1-oxoisoindolin-2-yl)piperidine-1-carboxylate (900 mg, 2.38 mmol, 1 eq) and 2-bromopropane (15.0 g, 121.96 mmol, 11.45 mL, 51.14 eq) in acetonitrile (40 mL) was added potassium carbonate (1.65 g, 11.92 mmol, 5 eq). The mixture was stirred at 80° C. for 12 h. The reaction mixture was filtered, and the filtrate was concentrated under reduced pressure to give a residue. The residue was purified by column chromatography (silicon dioxide, eluted with petroleum ether/ethyl acetate=5:1 to 1:1) to afford tert-butyl 4-(6-isopropoxy-5-nitro-1-oxoisoindolin-2-yl)piperidine-1-carboxylate (700 mg, 1.67 mmol, 70% yield) as a yellow solid. MS (ESI) m/z: 364.2 [M-tBu+H]⁺.

### Step 7:

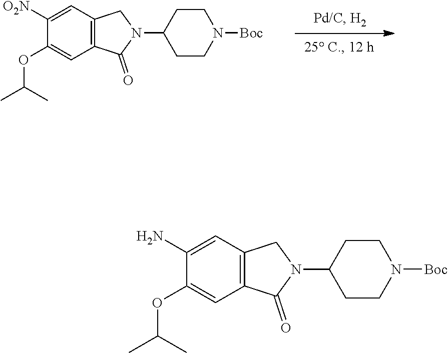 [SMILES: CC(C)Oc1cc2c(cc1[N+](=O)[O-])CN(C1CCN(C(=O)OC(C)(C)C)CC1)C2=O ; CC(C)Oc1cc2c(cc1N)CN(C1CCN(C(=O)OC(C)(C)C)CC1)C2=O]

To a solution of tert-butyl 4-(6-isopropoxy-5-nitro-1-oxoisoindolin-2-yl)piperidine-1-carboxylate (700 mg, 1.67 mmol, 1 eq) in 2,2,2-trifluoroethanol (15 mL) was added palladium on carbon (100 mg, 93.97 mmol, 10% purity, 0.0563 eq) under an atmosphere of nitrogen. The suspension was degassed under vacuum and purged with hydrogen gas several times. The mixture was stirred under hydrogen (15 psi) at 25° C. for 2 h. The reaction mixture was filtered, and the filtrate was concentrated under reduced pressure to give a residue. The crude product tert-butyl 4-(5-amino-6-isopropoxy-1-oxoisoindolin-2-yl)piperidine-1-carboxylate (649.5 mg, 1.67 mmol, >98% yield) afforded as a yellow solid was used in the subsequent reaction without further purification. MS (ESI) m/z: 334.2 [M-tBu+H]⁺.

### Step 8:

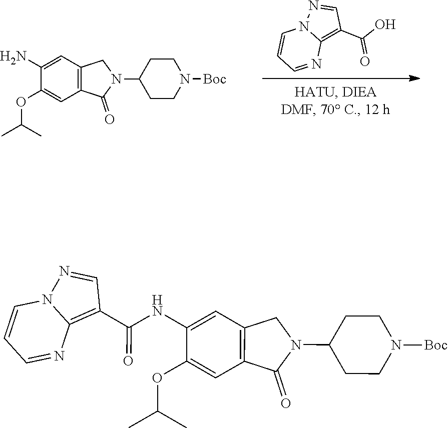 [SMILES: CC(C)Oc1cc2c(cc1N)CN(C1CCN(C(=O)OC(C)(C)C)CC1)C2=O ; O=C(O)c1cnn2cccnc12 ; CC(C)Oc1cc2c(cc1NC(=O)c1cnn3cccnc13)CN(C1CCN(C(=O)OC(C)(C)C)CC1)C2=O]

To a solution of tert-butyl 4-(5-amino-6-isopropoxy-1-oxoisoindolin-2-yl)piperidine-1-carboxylate (300 mg, 770.2 mmol, 1 eq) and pyrazolo[1,5-a]pyrimidine-3-carboxylic acid (200 mg, 1.23 mmol, 1.59 eq) in DMF (10 mL) was added diisopropylethylamine (300.0 mg, 2.32 mmol, 404.3 μL, 3.01 eq), followed by the addition of HATU (500 mg, 1.31 mmol, 1.71 eq) to the mixture. The mixture was allowed to stir at 70° C. for 12 h. The reaction mixture was poured into cold water (20 mL) and extracted with ethyl acetate (10 mL×3). The combined organic phase was washed with saturated brine (10 mL×3), dried over anhydrous sodium sulfate, filtered and concentrated under reduced pressure to give a residue. The residue was purified by column chromatography (silicon dioxide, eluted with ethyl acetate:methanol=0:1 to 10:1) to afford tert-butyl 4-(6-isopropoxy-1-oxo-5-(pyrazolo[1,5-a]pyrimidine-3-carboxamido)isoindolin-2-yl)piperidine-1-carboxylate (410 mg, 766.9 mmol, >98% yield) as a yellow solid. MS (ESI) m/z: 479.2 [M-tBu+H]⁺.

### Step 9:

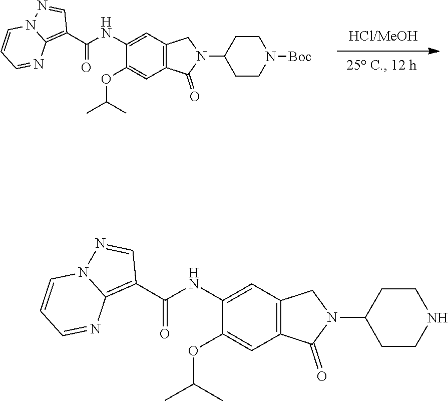 [SMILES: CC(C)Oc1cc2c(cc1NC(=O)c1cnn3cccnc13)CN(C1CCN(C(=O)OC(C)(C)C)CC1)C2=O ; CC(C)Oc1cc2c(cc1NC(=O)c1cnn3cccnc13)CN(C1CCNCC1)C2=O]

To a solution of tert-butyl 4-(6-isopropoxy-1-oxo-5-(pyrazolo[1,5-a]pyrimidine-3-carboxamido)isoindolin-2-yl)piperidine-1-carboxylate (410 mg, 766.9 mmol, 1 eq) in methanol (4 mL) was added a solution of hydrogen chloride in methanol (4 M, 8.0 mL, 41.7 eq). The mixture was stirred at 25° C. for 12 h. The reaction mixture was concentrated under reduced pressure, and the remaining residue was dissolved in 50 mL of methanol. The pH of the solution was then adjusted to 7-8 with the addition of a saturated aqueous solution of sodium bicarbonate solution. The mixture was then concentrated under reduced pressure to remove methanol, and the resulting residue was dissolved in ethanol (20 mL), filtered, and the filtrate was concentrated under reduced pressure to afford crude N-(6-isopropoxy-1-oxo-2-(piperidin-4-yl)isoindolin-5-yl)pyrazolo[1,5-a]pyrimidine-3-carboxamide (330 mg, 759.5 mmol, 99% yield) as a yellow solid which used in a subsequent transformation without further purification. MS (ESI) m/z: 435.3 [M+H]⁺.

### Preparation of Example 1: N-(2-(1-((1-(2-(2,6-dioxopiperidin-3-yl)-1,3-dioxoisoindolin-5-yl)piperidin-4-yl)methyl)piperidin-4-yl)-6-isopropoxy-1-oxoisoindolin-5-yl)pyrazolo[1,5-a]pyrimidine-3-carboxamide

### Step 1:

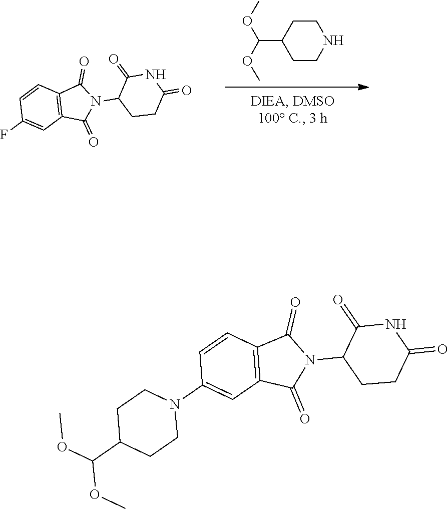 [SMILES: O=C1CCC(N2C(=O)c3ccc(F)cc3C2=O)C(=O)N1 ; COC(OC)C1CCNCC1 ; COC(OC)C1CCN(c2ccc3c(c2)C(=O)N(C2CCC(=O)NC2=O)C3=O)CC1]

To a solution of 2-(2,6-dioxo-3-piperidyl)-5-fluoro-isoindoline-1,3-dione (300 mg, 1.09 mmol, 1 eq) and 4-(dimethoxymethyl)piperidine (208 mg, 1.30 mmol, 1.2 eq) in dimethyl sulfoxide (5 mL) was added N,N-diisopropylethylamine (421 mg, 3.26 mmol, 0.6 mL, 3 eq). The mixture was stirred at 100° C. for 3 h. The mixture was poured into water (20 mL) and extracted with dichloromethane (20 mL×2), the organic phase was dried by anhydrous sodium sulfate, filtered and the filtrate was concentrated to give crude product. This crude product was purified by silica gel column chromatography (dichloromethane:methanol=100:1 to 50:1) to afford 5-[4-(dimethoxymethyl)-1-piperidyl]-2-(2,6-dioxo-3-piperidyl)isoindoline-1,3-dione (600 mg, crude) as a yellow solid. MS (ESI) m/z: 416.1 [M+H]⁺.

### Step 2:

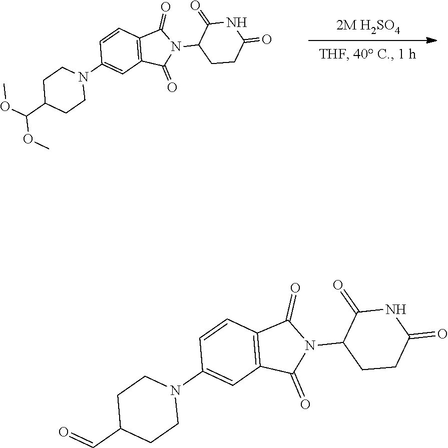 [SMILES: COC(OC)C1CCN(c2ccc3c(c2)C(=O)N(C2CCC(=O)NC2=O)C3=O)CC1 ; O=CC1CCN(c2ccc3c(c2)C(=O)N(C2CCC(=O)NC2=O)C3=O)CC1]

To a solution of 5-[4-(dimethoxymethyl)-1-piperidyl]-2-(2,6-dioxo-3-piperidyl)isoindoline-1,3-dione (450 mg, 1.08 mmol, 1 eq) in tetrahydrofuran (4 mL) was added sulfuric acid (2 M, 4 mL, 7 eq). The mixture was stirred at 40° C. for 1 h. The pH of the mixture was adjusted to 7 with a saturated aqueous solution of sodium bicarbonate, and extracted with ethyl acetate (30 mL×2). The organic phase was dried by anhydrous sodium sulfate, filtered, and the filtrate was concentrated to afford 1-[2-(2,6-dioxo-3-piperidyl)-1,3-dioxo-isoindolin-5-yl]piperidine-4-carbaldehyde (500 mg) as a yellow solid which was used in further transformations without further purification. MS (ESI) m/z: 370.3 [M+H]⁺. ¹H NMR (400 MHz, CDCl₃) δ: 9.72 (s, 1H), 8.12 (br s, 1H), 7.70 (d, J=8.4 Hz, 1H), 7.30 (d, J=2.3 Hz, 1H), 7.07 (dd, J=2.3, 8.6 Hz, 1H), 4.95 (dd, J=5.4, 12.3 Hz, 1H), 3.86 (td, J=4.2, 13.3 Hz, 2H), 3.19 (ddd, J=3.1, 10.5, 13.3 Hz, 2H), 2.94-2.70 (m, 3H), 2.60-2.52 (m, 1H), 2.18-2.11 (m, 1H), 1.86-1.84 (m, 1H), 1.85-1.76 (m, 1H).

### Step 3:

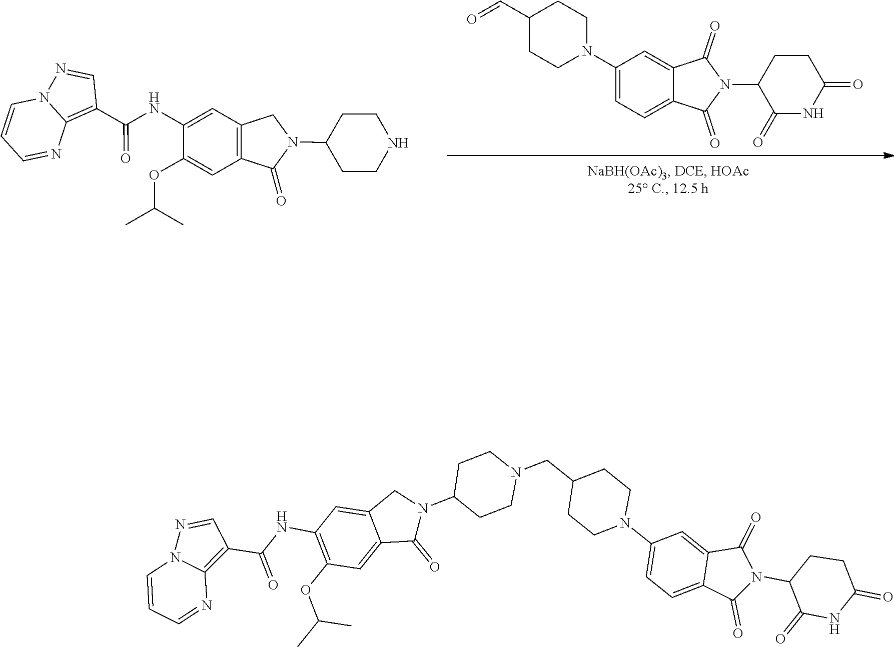 [SMILES: CC(C)Oc1cc2c(cc1NC(=O)c1cnn3cccnc13)CN(C1CCNCC1)C2=O ; O=CC1CCN(c2ccc3c(c2)C(=O)N(C2CCC(=O)NC2=O)C3=O)CC1 ; CC(C)Oc1cc2c(cc1NC(=O)c1cnn3cccnc13)CN(C1CCN(CC3CCN(c4ccc5c(c4)C(=O)N(C4CCC(=O)NC4=O)C5=O)CC3)CC1)C2=O]

To a solution of N-(6-isopropoxy-1-oxo-2-(piperidin-4-yl)isoindolin-5-yl)pyrazolo[1,5-a]pyrimidine-3-carboxamide (800 mg, 1.84 mmol, 1 eq) and 1-(2-(2,6-dioxopiperidin-3-yl)-1,3-dioxoisoindolin-5-yl)piperidine-4-carbaldehyde (700 mg, 1.90 mmol, 1.03 eq) in dichloroethane (10 mL) was added glacial acetic acid (330 mg, 5.50 mmol, 314.3 mL, 2.98 eq). The mixture was allowed to stir at 25° C. for 0.5 h prior to the addition of sodium borohydride acetate (1.56 g, 7.36 mmol, 4 eq). The reaction continued to stir at 25° C. for 12 h. The reaction mixture was poured into 20 mL of water and adjusted the pH to 7-8 with a saturated aqueous solution of sodium bicarbonate. The mixture was extracted with dichloromethane (10 mL×3) and the combined organic phase was washed with saturated brine (10 mL×3), dried over anhydrous sodium sulfate, filtered, and concentrated under reduced pressure to give a residue. The residue was purified by preparative HPLC (column: Phenomenex Luna C18 250×50 mm×15 m; mobile phase: [water (0.225% formic acid)-acetonitrile]; B %: 15%-45%, 20 min) to afford N-(2-(1-((1-(2-(2,6-dioxopiperidin-3-yl)-1,3-dioxoisoindolin-5-yl)piperidin-4-yl)methyl)piperidin-4-yl)-6-isopropoxy-1-oxoisoindolin-5-yl)pyrazolo[1,5-a]pyrimidine-3-carboxamide (516.4 mg, 629.2 mmol, 34% yield, 96% purity) as a yellow solid. MS (ESI) m/z: 788.3 [M+H]⁺. ¹H NMR (400 MHz, CDCl₃) δ=10.71 (s, 1H), 8.87-8.84 (m, 2H), 8.80 (s, 1H), 8.73 (dd, J=2.0, 4.0 Hz, 1H), 8.05 (s, 1H), 7.69 (d, J=8.8 Hz, 1H), 7.39 (s, 1H), 7.30 (d, J=2.4 Hz, 1H), 7.11-7.04 (m, 2H), 4.95 (dd, J=5.2, 12.0 Hz, 1H), 4.81 (td, J=6.0, 12.0 Hz, 1H), 4.34 (s, 2H), 4.32-4.21 (m, 1H), 3.98 (br d, J 12.8 Hz, 211), 3.03-2.72 (in, 711), 2.25 (br d, J=6.8 Hz, 211), 2.20-2.09 (in, 311), 1.97-1.76 (in, 711), 1.52 (d, J=6.4 Hz, 611), 1.34-1.26 (in, 211).

TABLE 1
The following compounds were prepared in an analogous synthetic sequence as
that described for the preparation of Example 1.

| | | ¹H NMR |
|---|---|---|
| | | All spectra acquired at 400 MHz |
| Example | MS | unless otherwise indicated |
| Example 1 | See Above | |
| 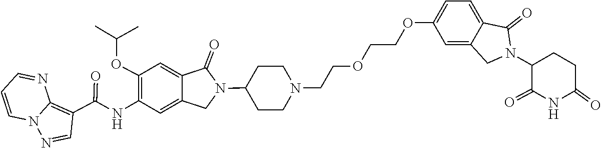 [SMILES: CC(C)Oc1cc2c(cc1NC(=O)c1cnn3cccnc13)CN(C1CCN(CCOCCOc3ccc4c(c3)CN(C3CCC(=O)NC3=O)C4=O)CC1)C2=O] | 765.2 | DMSO-d₆ δ = 10.96 (s, 1H), 10.74 (s, 1H), 9.39 (dd, J = 1.6, 7.2 Hz, 1H), 8.88 (dd, J = 1.6, 4.4 Hz, 1H), 8.73 (s, 2H), 8.19 (s, 1H), 7.63 (d, J = 8.4 Hz, 1H), 7.40-7.29 (m, 2H), 7.19 (d, J = 1.6 Hz, 1H), 7.08 (dd, J = 2.0, 8.4 Hz, 1H), 5.08 (dd, J = 5.2, 13.2 Hz, 1H), 4.94- 4.83 (m, 1H), 4.44-4.36 (m, 3H), |
| Example 2 | | 4.30-4.20 (m, 3H), 4.01-3.94 (m, |
| | | 1H), 3.79-3.76 (m, 2H), 3.61-3.59 |
| | | (m, 2H), 3.03-2.96 (m, 2H), 2.96- |
| | | 2.86 (m, 1H), 2.57-2.53 (m, 2H), |
| | | 2.44-2.32 (m, 1H), 2.17-2.07 (m, |
| | | 2H), 2.03-1.93 (m, 1H), 1.84-1.62 |
| | | (m, 4H), 1.43 (d, J = 6.0 Hz, 6H) |
| 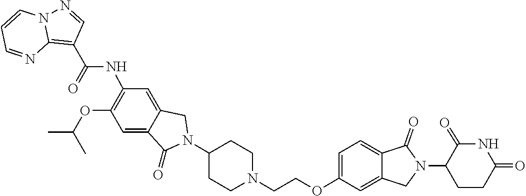 [SMILES: CC(C)Oc1cc2c(cc1NC(=O)c1cnn3cccnc13)CN(C1CCN(CCOc3ccc4c(c3)CN(C3CCC(=O)NC3=O)C4=O)CC1)C2=O] | 721.2 | DMSO-d₆ δ = 10.97 (s, 1H), 10.75 (s, 1H), 9.40 (dd, J = 1.2, 7.2 Hz, 1H), 8.89 (dd, J = 1.6, 4.0 Hz, 1H), 8.73 (s, 2H), 7.63 (d, J = 8.4 Hz, 1H), 7.43-7.30 (m, 2H), 7.20 (s, 1H), 7.08 (dd, J = 2.0, 8.4 Hz, 1H), 5.08 (dd, J = 5.2, 13.2 Hz, 1H), 4.96-4.82 (m, 1H), 4.46-4.37(m, 3H), 4.32-4.17 (m, 3H), 4.07-3.94 (m, 1H), 3.06 (d, J = 11.2 Hz, 2H), 2.96-2.85 (m, 1H), 2.77 (t, J = 5.2 Hz, 2H), 2.64-2.57 (m, 1H), 2.43-2.33 (m, 1H), 2.23-2.14 (m, 2H), |
| Example 3 | | 2.03-1.95 (m, 1H), |
| | | 1.88-1.76 (m, 2H), 1.74-1.66 (m, |
| | | 2H), 1.44 (d,7 = 6.0 Hz, 6H) |
| 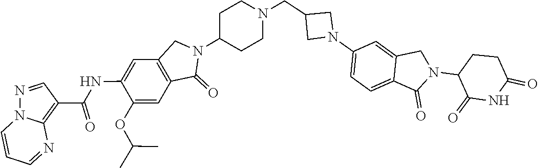 [SMILES: CC(C)Oc1cc2c(cc1NC(=O)c1cnn3cccnc13)CN(C1CCN(CC3CN(c4ccc5c(c4)CN(C4CCC(=O)NC4=O)C5=O)C3)CC1)C2=O] | 746.3 | DMSO-d₆ δ = 10.94 (s, 1H), 10.77 (s, 1H), 9.41 (dd, J = 1.6, 7.2 Hz, 1H), 8.90 (dd, J = 1.6, 4.4 Hz, 1H), 8.77 (s, 1H), 8.74 (s, 1H), 7.51 (d, J = 8.4 Hz, 1H), 7.40-7.34 (m, 2H), 6.54 (s, 1H), 6.50 (br d,J = 8.0 Hz, 1H), 5.04 (dd, J = 5.2, 13.2 Hz, 1H), 4.95- 4.87 (m, 1H), 4.44 (br s, 2H), 4.35-4.28 (m, 1H), 4.22-4.16 (m, 1H), 4.15-4.05 (m, 2H), 3.77-3.63 (m, 2H), |
| Example 4 | | 2.98-2.84 (m, 2H), |
| | | 2.65-2.52 (m, 8H), 2.45-2.34 (m, |
| | | 2H), 2.23-2.03 (m, 2H), 2.01-1.91 |
| | | (m, 2H), 1.44 (d, J = 5.6 Hz, 6H) |
| 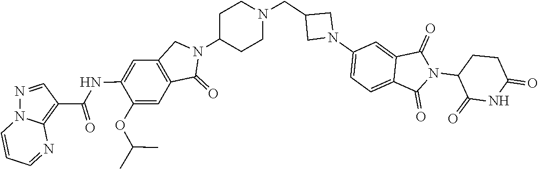 [SMILES: CC(C)Oc1cc2c(cc1NC(=O)c1cnn3cccnc13)CN(C1CCN(CC3CN(c4ccc5c(c4)C(=O)N(C4CCC(=O)NC4=O)C5=O)C3)CC1)C2=O] | 760.2 | DMSO-d₆ δ = 11.08 (s, 1H), 10.76 (s, 1H), 9.39 (dd, J = 6.8, 1.6 Hz, 1H), 8.89 (dd, J = 4.0, 1.6 Hz, 1H), 8.74 (d, J = 6.0 Hz, 2H), 7.63 (d, J = 8.0 Hz, 1H), 7.38-7.35 (m, 1H), 7.34 (s, 1H), 6.76 (d, J = 2.0 Hz, 1H), 6.64 (dd, J = 8.4, 2.0 Hz, 1H), 5.06 (dd, J =12.4,5.2 Hz, 1H), 4.94-4.86 (m, 1H), 4.43 (s, 2H), 4.14 (t, J = 8.00 |
| Example 5 | | Hz, 2H), 4.06-3.93 (m, 1H), |
| | | 3.74-3.65 (m, 2H), 3.07-2.82 |
| | | (m, 4H), 2.69-2.53 (m, 4H), |
| | | 2.17-1.96 (m, 3H), 1.88-1.73 (m, |
| | | 2H), 1.72-1.63 (m, 2H), 1.45 (d, |
| | | J = 6.0 Hz, 6H) |
| 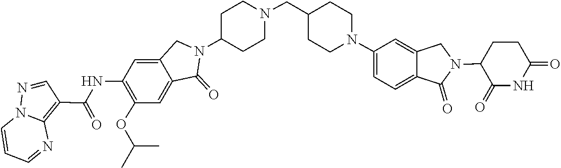 [SMILES: CC(C)Oc1cc2c(cc1NC(=O)c1cnn3cccnc13)CN(C1CCN(CC3CCN(c4ccc5c(c4)CN(C4CCC(=O)NC4=O)C5=O)CC3)CC1)C2=O] | 774.4 | CDCl3 δ = 10.71 (s, 1H) 8.84- 8.87 (m, 2 H) 8.80 (s, 1 H) 8.73 (dd, J = 4.0, 1.6 Hz, 1 H) 7.93 (s, 1 H) 7.73 (d, J = 8.4 Hz, 1H) 7.39 (s, 1H) 7.09 (dd, J = 6.8, 4.0 Hz, 1 H) 7.01 (dd, J = 8.4, 2.0 Hz, 1 H) 6.90 (s, 1 H) 5.18-5.24 (m, 1 H) 4.75- 4.85 (m, 1H) 4.38-4.46 (m, 1 H) 4.34 (s, 2 H) 4.23-4.31 (m, 2 H) 3.80-3.90 (m, 2 H) 3.47- |
| Example 6 | | 3.52 (m, 1 H) 2.93-3.04 (m, |
| | | 2 H) 2.84-2.92 (m, 3 H) 2.31- |
| | | 2.41 (m, 1 H) 2.10-2.30 (m, |
| | | 5H) 1.83-1.93 (m, 5H) 1.67- |
| | | 1.79 (m, 1 H) 1.52 (d, J = 6.0 |
| | | Hz, 6H) 1.27-1.41 (m, 3 H) |
| 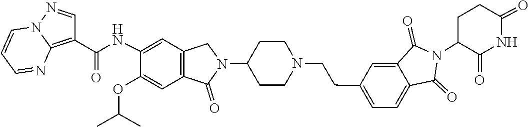 [SMILES: CC(C)Oc1cc2c(cc1NC(=O)c1cnn3cccnc13)CN(C1CCN(CCc3ccc4c(c3)C(=O)N(C3CCC(=O)NC3=O)C4=O)CC1)C2=O] | 719.2 | CDCl3 δ = 10.71 (s, 1H), 8.88- 8.83 (m, 2H), 8.81 (s, 1H), 8.73 (dd, J = 1.6, 4.0 Hz, 1H), 8.39 (br s, 1H),7.83 (d, J = 7.6 Hz, 1H), 7.76 (s, 1H), 7.61 (br d, J = 7.6 Hz, 1H), 7.38 (s, 1H), 7.08 (dd, J = 4.0, 6.8 Hz, 1H), 5.00 (dd, J = 5.2, 12.4 |
| Example 7 | | Hz, 1H), 4.84-4.77 (m, 1H), |
| | | 4.33 (s, 2H), 4.32-4.26 (m, |
| | | 1H), 3.12-2.69 (m, 9H), 2.33- |
| | | 2.12(m,3H), 1.92-1.79 (m, |
| | | 4H), 1.52 (d, J = 6.0 Hz, 6H) |
| 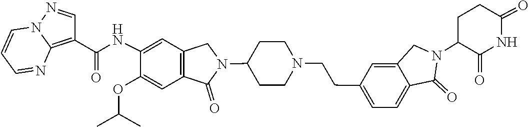 [SMILES: CC(C)Oc1cc2c(cc1NC(=O)c1cnn3cccnc13)CN(C1CCN(CCc3ccc4c(c3)CN(C3CCC(=O)NC3=O)C4=O)CC1)C2=O] | 705.2 | CDCl3 δ = 10.71 (s, 1H), 8.83- 8.88 (m, 2 H), 8.80 (s, 1 H), 8.73 (dd, J = 4.0, 1.6 Hz, 1 H), 8.05-8.11 (m, 1 H), 7.84 (d, J = 8.0 Hz, 1H), 7.34-7.40 (m, 3 H), 7.09 (dd, J = 6.8, 4.0 Hz, 1H), 5.24 (dd, J = 13.2,5.2 Hz, 1 H), 4.76-4.86 (m, 1H), 4.45- |
| Example 8 | | 4.55(m, 1H), 4.32-4.38 (m, |
| | | 3 H), 3.50 (s, 2 H), 3.09-3.21 |
| | | (m, 1H), 2.91-3.01 (m, 2 H), |
| | | 2.83-2.90 (m, 1 H), 2.66- |
| | | 2.79 (m, 1 H), 2.62-2.52 (m, 1 |
| | | H), 2.22-2.42 (m, 3 H), 1.84- |
| | | 1.98 (m, 3 H), 1.52 (d, J = 3.0 |
| | | Hz, 6H), 1.26-1.25 (m, 3 H) |
| 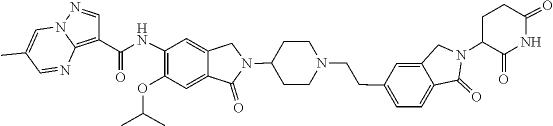 [SMILES: Cc1cnc2c(C(=O)Nc3cc4c(cc3OC(C)C)C(=O)N(C3CCN(CCc5ccc6c(c5)CN(C5CCC(=O)NC5=O)C6=O)CC3)C4)cnn2c1] | 719.3 | CDCl3 δ = 10.66 (s, 1H), 8.86 (s, 1H), 8.72 (s, 1H), 8.64 (dd, J = 1.2, 2.0 Hz, 1H), 8.58 (d, J = 2.4 Hz, 1H), 8.18 (br s, 1H), 7.83 (d, J = 8.0 Hz, 1H), 7.38 (s, 1H), 7.37-7.32 (m, 2H), 5.24 (dd, J = 5.2, 13.2 Hz, 1H), 4.84-4.76 (m, 1H), |
| Example 9 | | 4.53-4.47 (m, 1H), 4.37- |
| | | 4.32 (m, 3H), 3.77-3.74 (m, |
| | | 1H), 3.16-3.06 (m, 2H), 2.98- |
| | | 2.90 (m, 3H), 2.72-2.64 (m, |
| | | 2H), 2.51 (d, J = 0.4 Hz, 3H), |
| | | 2.41-2.19 (m, 4H), 1.93- |
| | | 1.83 (m, 5H), 1.52 (d, J = 6.0 |
| | | Hz, 6H) |
| 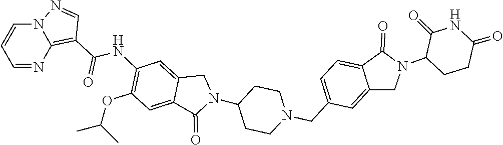 [SMILES: CC(C)Oc1cc2c(cc1NC(=O)c1cnn3cccnc13)CN(C1CCN(Cc3ccc4c(c3)CN(C3CCC(=O)NC3=O)C4=O)CC1)C2=O] | 691.2 | DMSO-d₆ δ = 11.05-10.95 (m, 1 H), 10.79-10.71 (m, 1 H), 9.40 (dd, J = 1.6, 7.2 Hz, 1H), 8.88 (dd, J = 1.6, 4.0 Hz, 1 H), 8.72 (s, 2 H), 7.70 (d, J = 8.0 Hz, 1 H), 7.57 (s, 1 H), 7.48 (d, J = 8.0 Hz, 1 H), 7.39- 7.29 (m, 2 H), 5.18-5.07 (dd, J = 5.2, 13.2 Hz, 1 H), |
| Example 10 | | 4.96-4.81 (m, 1 H), 4.51- |
| | | 4.39 (m, 3 H), 4.38-4.27 (d, J = |
| | | 17.2 Hz, 1 H), 4.07-3.93 |
| | | (m, 1 H), 3.61 (s, 2 H), 2.98- |
| | | 2.85 (m, 3 H), 2.63-2.56 (m, |
| | | 1 H), 2.42-2.36 (m, 1 H),2.18- |
| | | 2.07 (dd, J = 11.6, 22.4 Hz, |
| | | 2 H), 2.05-1.95 (m, 1 H), 1.89- |
| | | 1.73 (m, 2 H), 1.73-1.61 (m, |
| | | 2H), 1.46-1.39 (m, 6 H) |
| 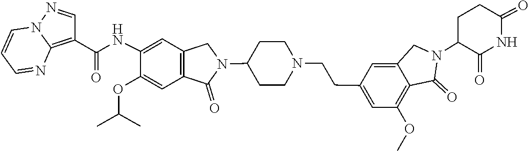 [SMILES: COc1cc(CCN2CCC(N3Cc4cc(NC(=O)c5cnn6cccnc56)c(OC(C)C)cc4C3=O)CC2)cc2c1C(=O)N(C1CCC(=O)NC1=O)C2] | 735.2 | DMSO-d₆ δ = 10.94 (s, 1 H), 10.75 (s, 1 H), 9.40 (d, J = 7.2 Hz, 1 H), 8.89 (d, J = 4.4 Hz, 1 H), 8.74 (s, 2 H), 8.27 (s, 1 H), 7.36 (dd, J = 6.8, 4.4 Hz, 1 H), 7.33 (s, 1 H), 7.00 (s, 1 H), 6.96 (s, 1 H), 5.02 (dd, = 13.2, 5.2 Hz, 1 H), 4.94- 4.86 (m, 1 H), 4.42 (s, 2 H), 4.36-4.28 (m, 1 H), 4.24- |
| Example 11 | | 4.14 (m, 1H), 4.04-3.96 (m, 1 |
| | | H), 3.87 (s, 3 H), 3.08-3.05 |
| | | (m, 1H), 2.95-2.89 (m, 1 H), |
| | | 2.88-2.82 (m, 2 H), 2.68- |
| | | 2.55 (m, 4 H), 2.39-2.30 (m, |
| | | l H), 2.17-2.08 (m, 2H), 1.99- |
| | | 1.91 (m, 1 H), 1.86-1.67 (m, |
| | | 4H), 1.44 (d, J = 6.0 Hz, 6 H) |
| 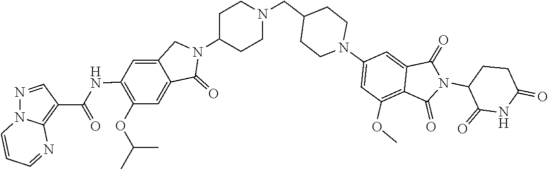 [SMILES: COc1cc(N2CCC(CN3CCC(N4Cc5cc(NC(=O)c6cnn7cccnc67)c(OC(C)C)cc5C4=O)CC3)CC2)cc2c1C(=O)N(C1CCC(=O)NC1=O)C2=O] | 818.4 | CDCl3 δ = 10.71 (s, 1H) 8.88- 8.82 (m, 2 H) 8.79 (s, 1 H) 8.75-8.71 (m, 1 H) 7.39 (s, 1 H)7.27 (s, 1H) 7.11-7.06 (m, 1 H) 6.99-6.94 (m, 1 H) 6.49- 6.39 (m, 1 H) 6.43 (d, J = 1.6 Hz, 1 H) 4.97-4.88 (m, 1 H) 4.85-4.76 (m, 1 H) 4.34 (s, 2 H) 4.30-4.21 (m, 1H) 3.98 (s, 4 H) 3.94 (s, 1 H) 3.06-2.95 (m, 4 H) 2.90-2.69 (m, 3 H) |
| Example 12 | | 2.30-2.23 (m, 2H) 2.19-2.08 |
| | | (m, 3 H) 1.96-1.89 (m, 2 H) |
| | | 1.87-1.79 (m, 4 H) 1.52 (s, 3 |
| | | H) 1.51 (s, 3 H) 1.34-1.26 (m, 2H) |
| 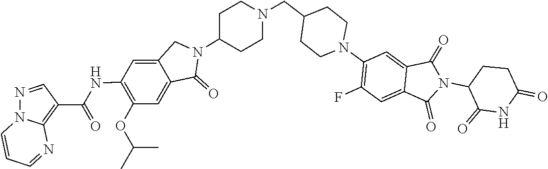 [SMILES: CC(C)Oc1cc2c(cc1NC(=O)c1cnn3cccnc13)CN(C1CCN(CC3CCN(c4cc5c(cc4F)C(=O)N(C4CCC(=O)NC4=O)C5=O)CC3)CC1)C2=O] | 806.3 | CDCl3 δ = 10.71 (s, 1H), 8.87- 8.84 (m, 2H), 8.80 (s, 1H), 8.73 (dd, J = 1.6, 4.0 Hz, 1H), 8.03 (s, 1H),7.47 (d, J = 11.2 Hz, 1H), 7.43-7.38 (m, 2H), 7.09 (dd, J = 4.0, 7.2 Hz, 1H), 4.94 (dd, J = 5.2, 12.4 Hz, 1H),4.81 (td, J = 6.0, 12.4 Hz, 1H), 4.35 (s, 2H), 4.33-4.24 (m, 1H), 3.73-3.65 (m, 2H), 3.03-2.97 (m, 2H), 2.92- |
| Example 13 | | 2.73 (m, 5H), 2.32-2.26 (m, |
| | | 2H), 2.21-2.12 (m, 3H), 1.95- |
| | | 1.83 (m, 6H), 1.78-1.70 (m, |
| | | 1H), 1.52 (d, J = 6.0 Hz, 6H), |
| | | 1.48-1.39 (m, 2H) |
| 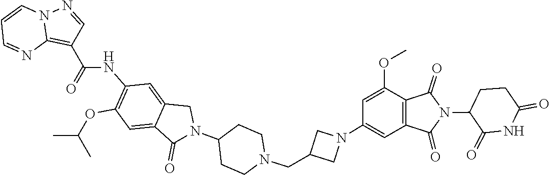 [SMILES: COc1cc(N2CC(CN3CCC(N4Cc5cc(NC(=O)c6cnn7cccnc67)c(OC(C)C)cc5C4=O)CC3)C2)cc2c1C(=O)N(C1CCC(=O)NC1=O)C2=O] | 790.3 | DMSO-d₆ δ = 11.04 (s, 1 H) 10.75 (s, 1 H) 9.37 (dd, J = 7.2 Hz 1.6 Hz, 1 H) 8.88 (dd, J = 4.0 Hz 1.2 Hz, 1H) 8.75 (s, 1 H) 8.72 (s, 1 H) 7.36-7.30 (m, 2 H) 6.35 (d, J = 1.2 Hz, 1 H) 6.08 (s, 1H) 5.02-4.95 (m, 1 H) 4.94-4.86 (m, 1 H) 4.42 (s, 2 H) 4.12 (J = 4.4 Hz, 2 H) 4.03-3.96 (m, 1 H) 3.88 (s, 3 H) 3.73-3.67 (m, 2H) 3.04- |
| Example 14 | | 2.80 (m, 5 H) 2.64-2.59 (m, 2 |
| | | H)2.54 (s, 1H) 2.12-2.04 (m, |
| | | 2 H) 2.00-1.93 (m, 1 H) 1.82- |
| | | 1.72 (m,2H) 1.71-1.62 (m, |
| | | 2H) 1.44 (d, J = 6.0 Hz, 6H) |
| 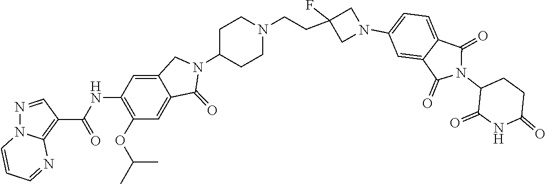 [SMILES: CC(C)Oc1cc2c(cc1NC(=O)c1cnn3cccnc13)CN(C1CCN(CCC3(F)CN(c4ccc5c(c4)C(=O)N(C4CCC(=O)NC4=O)C5=O)C3)CC1)C2=O] | 792.3 | DMSO-d₆ δ = 11.06 (s, 1H), 10.75 (s, 1H), 9.40 (dd, J = 1.6, 6.8 Hz, 1H), 8.89 (dd, J = 1.6, 4.0 Hz, 1H), 8.74 (d, J = 3.6 Hz, 2H), 8.22 (s, 1H), 7.69 (d, J = 8.0 Hz, 1H), 7.36 (dd, J = 4.0, 6.8 Hz, 1H), 7.32 (s, 1H), 6.92 (d, J= 1.6 Hz, 1H), 6.75 (dd, J = 2.0, 8.4 Hz, 1H), 5.11 (dd, J = 5.6, 12.8 Hz, 4.03 -3.93 (m, 1H),2.98 (br t, J = 10.4 Hz, 2H), 2.93-2.84 |
| Example 15 | | (m, 1H), 2.63-2.52 (m, 4H), |
| | | 2.18-1.97 (m, 5H), 1.82- |
| | | 1.63 (m, 4H), 1.44 (d, J = 6.0 |
| | | Hz, 6H) |
| 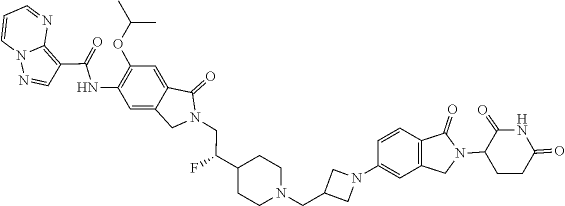 [SMILES: CC(C)Oc1cc2c(cc1NC(=O)c1cnn3cccnc13)CN(C[C@@H](F)C1CCN(CC3CN(c4ccc5c(c4)CN(C4CCC(=O)NC4=O)C5=O)C3)CC1)C2=O] | 792.2 | CDCl3 δ = 10.72 (s, 1 H), 8.89- 8.81 (m, 2 H), 8.79 (s, 1 H), 8.72 (dd, J = 1.6, 4.0 Hz, 1H), 8.32-8.18 (m, 1 H), 7.67 (d, J = 8.4 Hz, 1 H), 7.38 (s, 1 H), 7.09 (dd, J = 4.0, 6.8 Hz, 1 H), 6.42 (m, 1 H), 6.37 (s, 1 H), 5.17 (dd, J = 5.2, 13.2 Hz, 1 H), 4.79 (m, 1 H), 4.66-4.42 (m, 3 H), 4.37 (d, J = 16 Hz, 1 H), 4.28-4.18 (m, 1 H), 4.12 (m, 2 H), 4.05-3.72 (m, 3 H), 3.66 (s, 2 H), 3.37-3.01 (m, 3 |
| Example 16 | | H), 2.98-2.71 (m, 4 H), 2.43- |
| | | 2.13 (m, 4H), 2.09-1.91 (m, |
| | | 2H), 1.72 (m, 2 H), 1.52 (d, J = |
| | | 6.0 Hz, 6 H) |
| 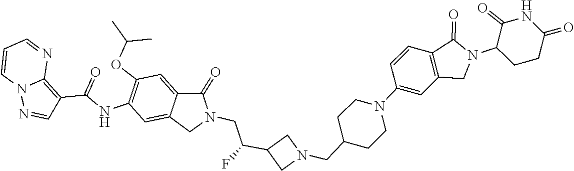 [SMILES: CC(C)Oc1cc2c(cc1NC(=O)c1cnn3cccnc13)CN(C[C@@H](F)C1CN(CC3CCN(c4ccc5c(c4)CN(C4CCC(=O)NC4=O)C5=O)CC3)C1)C2=O] | 792.3 | DMSO-d₆ δ = 10.93 (s, 1 H), 10.76 (s, 1 H), 9.40 (dd, J = 6.8, 1.6 Hz, 1H), 8.90 (dd, J = 4.4,1.6 Hz, 1H), 8.74 (s, 2H), 7.48 (d, J = 8.4 Hz, 1 H), 7.34- 7.39 (m, 2 H), 7.00-7.04 (m, 2 H), 5.03 (dd, J = 13.2, 5.2 Hz, 1H), 4.83-4.98 (m, 2 H), 4.51 (d, J = 4.0 Hz, 2 H), 4.16-4.33 (m, 2H), 3.84 (d, J = 12.0 Hz, 2H), 3.57-3.75 (m, 2 H), 3.29 |
| Example 17 | | (s, 3 H), 2.86-3.04 (m, 3 H), |
| | | 2.74-2.82 (m, 2 H), 2.58 (d, J = |
| | | 16.4 Hz, 2 H), 2.26 (s, 2 H), |
| | | 1.91-1.98 (m, 1 H), 1.73 (d, J = |
| | | 12.8 Hz, 2H), 1.45 (d, J = |
| | | 6.0 Hz, 6 H), 1.12-1.24 (m, 3 H) |
| 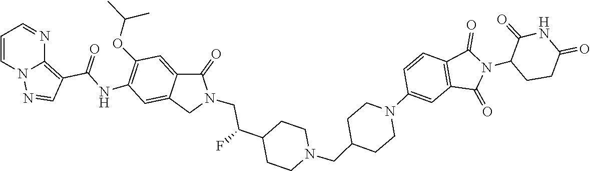 [SMILES: CC(C)Oc1cc2c(cc1NC(=O)c1cnn3cccnc13)CN(C[C@@H](F)C1CCN(CC3CCN(c4ccc5c(c4)C(=O)N(C4CCC(=O)NC4=O)C5=O)CC3)CC1)C2=O] | 834.3 | DMSO-d₆ δ = 11.09 (s, 1H), 10.77 (s, 1H), 9.41 (dd, J = 1.6, 7.2 Hz, 1H), 8.90 (dd, J = 1.6, 4.4 Hz, 1H), 8.75 (s, 2H), 8.25 (s, 1H), 7.65 (d, J = 8.4 Hz, 1H), 7.40-7.34 (m, 2H), 7.31 (d, J = 2.0 Hz, 1H), 7.23 (dd, J = 2.0, 8.4 Hz, 1H), 5.07 (dd, J = 5.2, 12.8 Hz, 1H), 4.92 (quin, J = 6.0 Hz, 1H), 4.68-4.44 (m, 3H), 4.04 (br d, |
| Example 18 | | J = 12.4 Hz, 2H), 3.89-3.68 |
| | | (m, 3H), 3.00-2.92 (m, 2H), |
| | | 2.91-2.85 (m, 2H), 2.63-2.52 |
| | | (m, 2H), 2.12 (br d, J = 6.8 |
| | | Hz, 2H), 2.06-1.98 (m, 1H), |
| | | 1.87-1.68 (m, 7H), 1.63-1.50 |
| | | (m, 1H), 1.45 (d, J = 6.0 Hz, |
| | | 6H), 1.40-1.30 (m, 2H), 1.19- |
| | | 1.07 (m, 2H). |
| 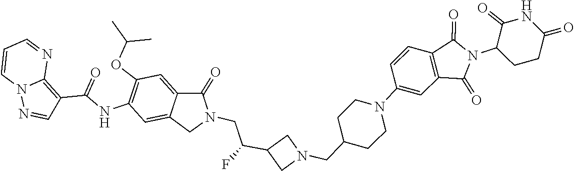 [SMILES: CC(C)Oc1cc2c(cc1NC(=O)c1cnn3cccnc13)CN(C[C@@H](F)C1CN(CC3CCN(c4ccc5c(c4)C(=O)N(C4CCC(=O)NC4=O)C5=O)CC3)C1)C2=O] | 806.3 | DMSO-ds 6 = 11.08 (s, 1 H) 10.75 (s, 1 H) 9.40 (d, J = 7.2 Hz, 1 H) 8.89 (br d, J = 3.6 Hz, 1 H) 8.73 (s, 2 H) 7.29-7.45 (m, 2 H) 6.96 (t, J = 8.0 Hz, 1 H) 6.65-6.79 (m, 2 H) 5.32 (br dd, J = 12.8, 4.8 Hz, 1 H) 4.81-5.01 (m, 1H) 4.38 (s, 2 H) 3.98 (br s, 1 H) 3.91 (br s, 2 H) 3.60-3.71 (m, 3 H) 3.59 (s, 3 H) 2.82-2.99 (m, 1 H) |
| Example 19 | | 2.58-2.76 (m, 2H) 2.45 (br s, |
| | | 1 H) 2.16 (s, 3 H) 2.00 (br d, |
| | | J = 6.85 Hz, 1 H) 1.78 (br s, 4 |
| | | H) 1.58-1.68 (m, 2 H) 1.49 (br s, |
| | | 2H) 1.44 (d, J = 6.0Hz, 6 H). |
| 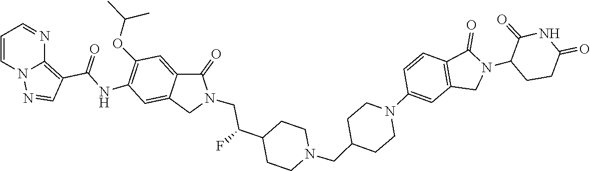 [SMILES: CC(C)Oc1cc2c(cc1NC(=O)c1cnn3cccnc13)CN(C[C@@H](F)C1CCN(CC3CCN(c4ccc5c(c4)CN(C4CCC(=O)NC4=O)C5=O)CC3)CC1)C2=O] | 820.3 | DMSO-d₆ δ = 10.94 (s, 1 H) 10.75 (s, 1H) 9.39 (dd, J = 7.2, 1.6 Hz, 1 H) 8.88 (dd, J = 4.4, 1.6 Hz, 1H) 8.73 (s, 2H) 7.49 (d, J = 9.2 Hz, 1 H) 7.32-7.38 (m, 2 H) 7.00-7.05 (m, 2 H) 5.04 (dd, J = 13.6, 5.2 Hz, 1 H) 4.90 (dt, J = 12.0, 6.0 Hz, 1 H) 4.42-4.69 (m, 3 H) 4.17-4.33 (m, 2 H) 3.67-3.88 (m, 4 H) 2.77-2.94 (m, 5H) 2.51-2.63 |
| Example 20 | | (m, 2 H) 2.30-2.41 (m, 1 H) |
| | | 2.11 (br s, 2H) 1.91-1.99 (m, |
| | | 1 H) 1.68-1.82 (m, 6H) 1.51- |
| | | 1.60 (m, 1 H) 1.44 (d, J = 6.0 |
| | | Hz, 6 H) 1.27-1.37 (m, 2 H) |
| | | 1.12 -1.21 (m, 2H) |
| 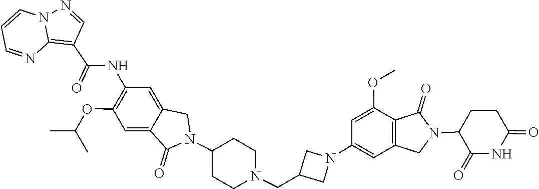 [SMILES: COc1cc(N2CC(CN3CCC(N4Cc5cc(NC(=O)c6cnn7cccnc67)c(OC(C)C)cc5C4=O)CC3)C2)cc2c1C(=O)N(C1CCC(=O)NC1=O)C2] | 776.3 | DMSO-d₆ δ = 10.90 (s, 1H), 10.76 (s, 1H), 9.40 (dd, J = 1.6, 7.2 Hz, 1H), 8.89 (dd, J = 1.6, 4.0 Hz, 1H), 8.77-8.71 (m, 2H), 7.39-7.32 (m, 2H), 6.06 (s, 1H), 5.92 (s, 1H), 4.98- 4.85 (m, 2H), 4.42 (s, 2H), 4.20 (d, J = 17.2 Hz, 1H),4.10- 3.95 (m, 4H), 3.81 (s, 3H), 3.58 (br t, J = 6.0 Hz, 2H), 3.01-2.82 (m, 4H), 2.64- 2.54 (m, 3H), 2.32-2.23 (m, |
| Example 21 | | 1H), 2.09 (br t, J = 11.2 Hz, |
| | | 2H), 2.01-1.67 (m, 5H), 1.44 |
| | | (d, J = 6.0 Hz, 6H) |
| 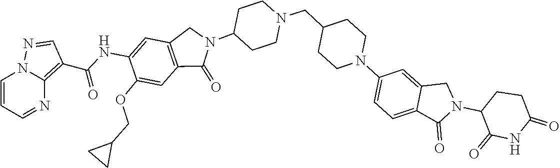 [SMILES: O=C1CCC(N2Cc3cc(N4CCC(CN5CCC(N6Cc7cc(NC(=O)c8cnn9cccnc89)c(OCC8CC8)cc7C6=O)CC5)CC4)ccc3C2=O)C(=O)N1] | 786.3 | DMSO-d₆ δ = 10.94 (s, 1 H) 10.82 (s, 1 H) 9.40 (d, J = 8 Hz, 1 H) 8.89 (s, 1 H) 8.77 (s, 2 H) 7.46-7.57 (m, 1 H)7.31- 7.37 (m, 1 H) 7.26 (s, 1 H) 7.02-7.08 (m, 2 H) 5.00-5.08 (m, 1 H) 4.40 (s, 2 H) 4.28- 4.37 (m, 1H) 4.17-4.25 (m, 1 H) 4.04 (s, 2H) 3.95-4.03 (m, 1H) 3.84-3.92 (m, 2 H) 2.93- 2.97 (m, 3 H) 2.81-2.87 (m, |
| Example 22 | | 2H) 1.98-2.04 (m, 3 H) 1.77- |
| | | 1.84 (m, 5 H) 1.67-1.71 (m, |
| | | 2H) 1.42-1.53 (m, 2H) 1.17- |
| | | 1.25(m, 3 H) 0.67-0.72 (m, |
| | | 2 H) 0.43-0.47 (m, 2 H) |
| 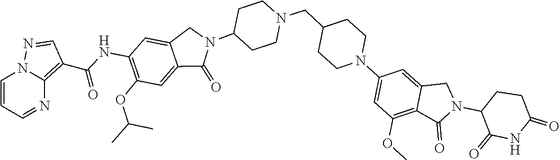 [SMILES: COc1cc(N2CCC(CN3CCC(N4Cc5cc(NC(=O)c6cnn7cccnc67)c(OC(C)C)cc5C4=O)CC3)CC2)cc2c1C(=O)N(C1CCC(=O)NC1=O)C2] | 804.5 | CDCl3 δ = 10.71 (s, 1H), 8.87-8.83 (m, 2H), 8.79 (s, 1H), 8.73 (dd, J = 4.0 Hz, J = 2.0 Hz, 1H), 7.89 (s, 1H), 7.39 (s, 1H), 7.11-7.07 (m, 1H), 6.50-6.47 (m, 1H), 6.38 (d, J = 1.2Hz, 1H), 5.18-5.11 (m, 1H), 4.85-4.76 (m, 1H), 4.39- 4.33 (m, 3H), 4.31-4.25 (m, 1H), 4.23-4.17 (m, 1H), 3.96 |
| Example 23 | | (s, 3H), 3.88-3.81 (m, 2H), |
| | | 3.04-2.97 (m, 2H), 2.93-2.85 |
| | | (m, 4H), 2.35-2.28 (m, 1H), |
| | | 2.26 (d, J = 6.8 Hz, 2H), 2.20- |
| | | 2.12 (m, 3H), 1.92 -1.83 (m, |
| | | 6H), 1.53 (s, 3H), 1.51 (s, 3H), |
| | | 1.40 -4.28 (m, 3H) |
| 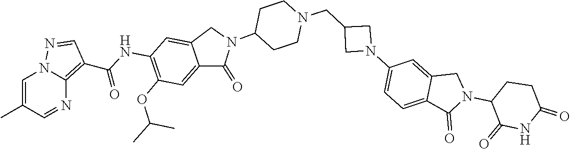 [SMILES: Cc1cnc2c(C(=O)Nc3cc4c(cc3OC(C)C)C(=O)N(C3CCN(CC5CN(c6ccc7c(c6)CN(C6CCC(=O)NC6=O)C7=O)C5)CC3)C4)cnn2c1] | 760.5 | CDCl3 δ = 10.67 (s, 1H), 8.85 (s, 1H), 8.71 (s, 1H), 8.64 (dd, J = 1.2, 2.4 Hz, 1H), 8.58 (d, J = 2.0 Hz, 1H), 8.01 (br s, 1H), 7.70 (d, J = 8.4 Hz, 1H), 7.38 (s, 1H), 6.46 (dd, J = 1.6, 8.4 Hz, 1H), 6.38 (d, J = 0.8 Hz, 1H), 5.21 (dd, J = 5.2, 13.2 Hz, 1H), 4.85-4.76 (m, 1H), 4.43-4.37 (m, 1H), 4.34 |
| Example 24 | | (s, 2H), 4.32-4.27 (m, 1H), |
| | | 4.27-4.21 (m, 1H), 4.12 (t, J = |
| | | 7.2 Hz, 2H), 3.69-3.63 (m, |
| | | 2H), 3.11-3.01 (m, 3H), 2.96- |
| | | 2.82 (m,2H), 2.80-2.75 (m, |
| | | 2H), 2.51 (s, 3H), 2.37-2.26 |
| | | (m, 3H), 2.24-2.18 (m, 1H), |
| | | 1.95-1.86 (m, 4H), 1.52 (d, J = |
| | | 6.0 Hz, 6H) |
| 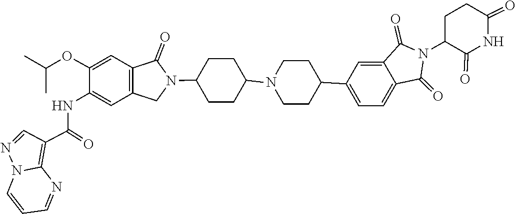 [SMILES: CC(C)Oc1cc2c(cc1NC(=O)c1cnn3cccnc13)CN(C1CCC(N3CCC(c4ccc5c(c4)C(=O)N(C4CCC(=O)NC4=O)C5=O)CC3)CC1)C2=O] | 773.3 | DMSO-d₆ δ = 11.13 (br s, 1H), 10.76 (s, 1H), 9.41 (br d, 1H, J = 7.1 Hz), 8.9-8.9 (m, 1H), 8.74 (s, 2H), 7.7-7.9 (m, 3H), 7.3-7.4 (m, 2H), 5.1-5.2 (m, 1H), 4.8-5.0 (m, 1H), 4.3-4.5 (m, 2H), 3.9-4.1 (m, 1H), 3.0- 3.1 (m, 2H), 2.8-3.0 (m, 2H), 2.7-2.8 (m, 2H), 2.0-2.1 (m, 2H), 1.8-2.0 (m, 7H), 1.6-1.8 (m, 4H), 1.4-1.6 (m, 9H) |
| Example 25 | | |
| 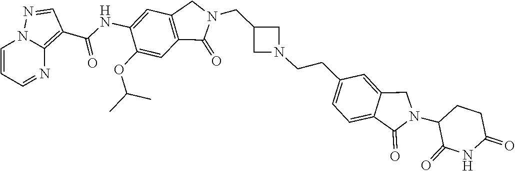 [SMILES: CC(C)Oc1cc2c(cc1NC(=O)c1cnn3cccnc13)CN(CC1CN(CCc3ccc4c(c3)CN(C3CCC(=O)NC3=O)C4=O)C1)C2=O] | 691.4 | DMSO-d₆ δ = 10.98 (s, 1 H), 10.75 (s, 1 H), 9.40 (dd, J = 6.8, 1.6 Hz, 1H), 8.89 (dd, J = 4.4, 1.6 Hz, 1 H), 8.70-8.76 (m, 2H), 8.20 (s, 1H),7.62 (d, J = 8.0 Hz, 1H), 7.45 (s, 1 H), 7.30-7.38 (m, 3 H), 5.10 (dd, J = 13.2,5.2 Hz, 1H), 4.89 (dt, J = 12.0, 6.0 Hz, 1 H), 4.39- 4.41 (m, 2 H), 4.25-4.32 (m, 1 H), 3.68 (d, J = 7.2 Hz, 2 H), |
| Example 26 | | 3.34 (s, 2 H), 2.97 (t, J = 6.8 |
| | | Hz, 2 H), 2.86-2.92 (m, 1 H), |
| | | 2.68 (s, 4 H), 2.52-2.63 (m, 2 |
| | | H), 2.31-2.46 (m, 2 H), 1.97-2.02 |
| | | (m, 1H), 1.44 (d, J = 6.0 Hz, 6 H) |
| 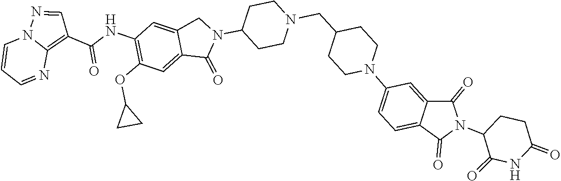 [SMILES: O=C1CCC(N2C(=O)c3ccc(N4CCC(CN5CCC(N6Cc7cc(NC(=O)c8cnn9cccnc89)c(OC8CC8)cc7C6=O)CC5)CC4)cc3C2=O)C(=O)N1] | 786.2 | CDCl3 δ = 10.55 (s, 1H), 8.85 (dd, J = 1.6, 7.2 Hz, 1H), 8.81- 8.77 (m, 2H), 8.75 (dd, J = 1.6, 4.0 Hz, 1H), 8.12-8.04 (m, 1H), 7.71 (s, 1H), 7.69 (d, J = 8.4 Hz, 1H), 7.30 (d, J = 2.0 Hz, 1H), 7.11-7.04 (m, 2H), 4.95 (dd, J = 5.2, 12.0 Hz, 1H), 4.38-4.32 (m, 2H), 4.32-4.28 (m, 1H), 4.05-3.93 (m, 3H), 3.03-2.69 (m, 7H), 2.26 |
| Example 27 | | (d, J = 6.8 Hz, 2H), |
| | | 2.20-2.10 (m, 3H), 1.95- |
| | | 1.78 (m, 7H), 1.38-1.24 (m, |
| | | 4H), 0.92-0.82 (m, 2H) |
| 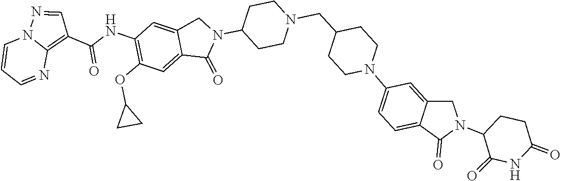 [SMILES: O=C1CCC(N2Cc3cc(N4CCC(CN5CCC(N6Cc7cc(NC(=O)c8cnn9cccnc89)c(OC8CC8)cc7C6=O)CC5)CC4)ccc3C2=O)C(=O)N1] | 772.2 | CDCl3 δ = 10.55 (s, 1H), 8.85 (dd, J = 1.6, 7.2 Hz, 1H), 8.80- 8.77 (m, 2H), 8.75 (dd, J = 1.6, 4.0 Hz, 1H), 7.97 (s, 1H), 7.73 (d, J = 8.4 Hz, 1H), 7.71 (s, 1H), 7.09 (dd, J = 4.4, 7.2 Hz, 1H), 7.01 (dd, J = 2.0, 8.4 Hz, 1H), 6.90 (s, 1H), 5.21 (dd, J = 5.2, 13.2 Hz, 1H), 4.45-4.39 (m, 1H), 4.35 (s, 2H), 4.32-4.24 (m, 2H), 4.05-3.99 (m, 1H), 3.86 (br d,J = |
| Example 28 | | 13.2 Hz, 2H), 3.04-2.97 (m, |
| | | 2H), 2.92-2.83 (m, 4H), 2.37- |
| | | 2.31 (m, 1H), 2.26 (br d, J = |
| | | 7.2 Hz, 2H), 2.23-2.12 (m, |
| | | 3H), 1.93-1.83 (m, 6H), 1.80- |
| | | 1.72 (m, 1H), 1.39-1.30 (m, |
| | | 2H), 0.99-0.91 (m, 4H) |
| 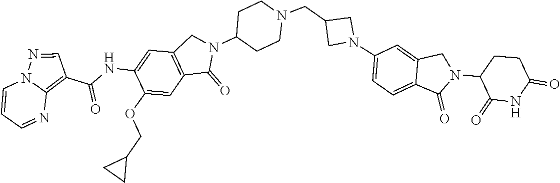 [SMILES: O=C1CCC(N2Cc3cc(N4CC(CN5CCC(N6Cc7cc(NC(=O)c8cnn9cccnc89)c(OCC8CC8)cc7C6=O)CC5)C4)ccc3C2=O)C(=O)N1] | 758.3 | DMSO-d₆ δ = 10.93 (s, 1 H) 10.82 (s, 1H) 9.39 (dd, J = 6.8, 1.6 Hz, 1 H) 8.89 (dd, J = 4.0, 1.6 Hz, 1H) 8.74 (s, 2H) 7.48 (d, J = 8.0 Hz, 1 H) 7.32-7.36 (m, 1 H) 7.26 (s, 1 H) 6.45-6.53 (m, 2 H) 4.99-5.08 (m, 1 H) 4.42 (s, 2 H) 4.27-4.33 (m, 1 H) 4.15-4.21 (m, 1 H) 3.98 (br s, 5H) 3.54-3.60 (m, 2H) 2.91-3.01 (m, 4 H) 2.59-2.64 (m, 4 H) 2.05-2.13 (m, 2 H) |
| Example 29 | | 1.91-1.99 (m, 1 H) 1.75-1.84 |
| | | (m, 2 H) 1.66-1.72 (m, 2 H) |
| | | 1.42-1.49 (m, 1H) 0.65-0.73 |
| | | (m, 2 H) 0.40-0.49 (m, 2 H) |
| 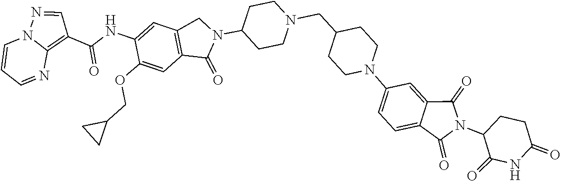 [SMILES: O=C1CCC(N2C(=O)c3ccc(N4CCC(CN5CCC(N6Cc7cc(NC(=O)c8cnn9cccnc89)c(OCC8CC8)cc7C6=O)CC5)CC4)cc3C2=O)C(=O)N1] | 800.3 | DMSO-d₆ δ = 11.07 (s, 1 H) 10.82 (s, 1 H) 9.40 (dd, J = 6.8, 1.6 Hz, 1 H) 8.89 (dd, J = 4.0, 1.6 Hz, 1 H) 8.72-8.76 (m, 2 H) 8.30 (s, 1H)7.65 (d, J = 8.4 Hz, 1 H) 7.30-7.36 (m, 2 H) 7.21-7.27 (m, 2 H) 5.06 (dd, J = 12.8, 5.2 Hz, 1H) 4.43 (s, 2 H) 3.95-4.13 (m, 5 H) 2.82- 3.04 (m, 6 H) 2.13-2.22 (m, 2 H) 1.96-2.06 (m, 3 H) 1.75- 1.88 (m, 5 H) 1.63-1.73 (m, 2 |
| Example 30 | | H) 1.39-1.51 (m, 1 H) 1.09- |
| | | 1.30 (m, 3 H) 0.64-0.73 (m, 2 |
| | | H) 0.41-0.49 (m, 2H) |
| 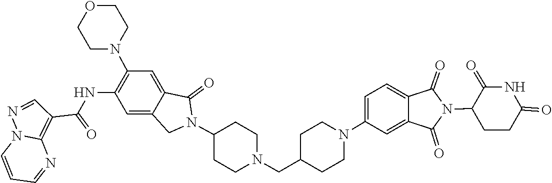 [SMILES: O=C1CCC(N2C(=O)c3ccc(N4CCC(CN5CCC(N6Cc7cc(NC(=O)c8cnn9cccnc89)c(N8CCOCC8)cc7C6=O)CC5)CC4)cc3C2=O)C(=O)N1] | 815.4 | DMSO-d₆ δ = 11.08 (s, 1 H), 10.91 (s, 1 H), 9.41 (dd, J = 1.6, 6.8 Hz, 1H), 9.00 (dd, J = 1.6, 4.4 Hz, 1H), 8.76 (d, J = 3.6 Hz, 2 H), 7.66 (d, J = 8.4 Hz, 1 H), 7.58 (s, 1 H), 7.42- 7.37 (m, 1 H), 7.32 (s, 1 H), 7.24 (dd, J =2.2, 8.8 Hz, 1 H), 5.07 (dd, J = 5.6, 12.8 Hz, 1 H), 4.47 (s, 2 H), 4.10-3.97 (m, 3 H), 3.94-3.86 (m, 4 H), d, J = 6.4 Hz, 2 H), 2.02 (br t, |
| Example 31 | | J = 10.8 Hz, 3 H), 1.91-1.63 |
| | | (m, 8H), 1.16 (br d, J = 11.6 |
| | | Hz, 2 H) |
| 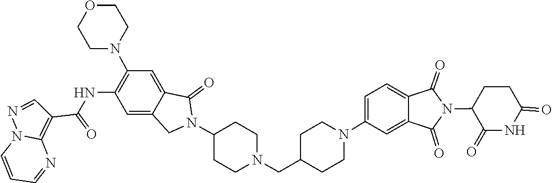 [SMILES: O=C1CCC(N2C(=O)c3ccc(N4CCC(CN5CCC(N6Cc7cc(NC(=O)c8cnn9cccnc89)c(N8CCOCC8)cc7C6=O)CC5)CC4)cc3C2=O)C(=O)N1] | 801.4 | MeOD δ = 9.18 (d, J = 7.2 Hz, 1 H), 8.99 (d, J = 4.0 Hz, 1 H), 8.84 (s, 1 H), 8.73 (s, 1 H), 7.74 (s, 1 H), 7.65 (d, J = 9.0 Hz, 1H), 7.33 (dd, J = 4.0, 7.0 Hz, 1H), 7.17-7.05 (m, 3 H), 5.13-5.09 (m, 1 H), 4.90- 4.89 (m, 4 H), 4.89-4.89 (m, 1H), 4.55 (s, 2 H), 4.48-4.38 (m, 3 H), 4.07-4.00 (m, 5 H), 3.80 (br d, J = 14.2 Hz, 2 H), 3.14 (br d, J = 6.4 Hz, 2 H), |
| Example 32 | | 3.00 (br d, J = 4.4 Hz, 5 H), |
| | | 2.84-2.73 (m, 1 H), 2.53- |
| | | 2.37 (m, 2 H), 2.29 (br d, J = |
| | | 11.6 Hz, 2H), 2.18 (br d, J = |
| | | 11.6 Hz, 4H), 1.94 (brd, J = |
| | | 11.6 Hz, 2H), 1.48 (brd, J = |
| | | 10.4 Hz, 2 H) |
| 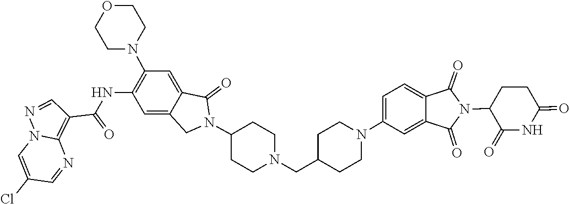 [SMILES: O=C1CCC(N2C(=O)c3ccc(N4CCC(CN5CCC(N6Cc7cc(NC(=O)c8cnn9cc(Cl)cnc89)c(N8CCOCC8)cc7C6=O)CC5)CC4)cc3C2=O)C(=O)N1] | 849.3 | DMSO-d₆ δ = 10.79 (s, 1 H), 8.92 (d, J = 2.4 Hz, 1 H), 8.86 (s, 1 H), 8.82 (s, 1 H), 8.76 (d, J = 2.4 Hz, 1H), 8.31 (s, 1H), 8.19 (s, 1 H), 7.74 (s, 1 H), 7.69 (d, J = 8.56 Hz, 1 H), 7.29 (d, J = 2.4 Hz, 1 H), 7.06 (dd, J = 8.56, 2.4 Hz, 1 H), 4.95 (dd, J = 12.4, 5.2 Hz, 1 H), 4.39 (s, 2H), 4.34 (s, 1 H), 3.99-4.02 (m, 4 H), 3.96 (s, 1 H), 3.15 (s, 2 H), 2.95-3.05 (m, 6 H), 2.77-2.89 (m, 2 H), |
| Example 33 | | 2.26-2.47 (m, 4 H), 2.08-2.18 |
| | | (m, 2 H), 1.84-2.00 (m, 8 H), |
| | | 1.35 (d, J= 12.0 Hz, 2H). |
| 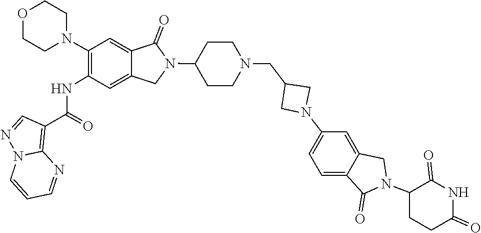 [SMILES: O=C1CCC(N2Cc3cc(N4CC(CN5CCC(N6Cc7cc(NC(=O)c8cnn9cccnc89)c(N8CCOCC8)cc7C6=O)CC5)C4)ccc3C2=O)C(=O)N1] | 773.4 | CDCl3 δ = 10.94-10.89 (m, 1H), 8.90-8.86 (m, 2H), 8.85-8.81 (m, 2H), 8.09-7.98 (m, 1H), 7.74-7.68 (m, 2H), 7.13 (dd, J = 4.0, 6.4 Hz, 1H), 6.49- 6.41 (m, 2H), 5.23-5.16 (m, 1H), 4.48-4.37 (m, 3H), 4.29- 4.23 (m, 2H), 4.04-3.99 (m, 4H), 3.85-3.78 (m, 1H), 3.74- 3.58 (m, 2H), 3.45-3.29 (m, 2H), 3.02-2.98 (m, 4H), 2.95-2.78 (m, 5H), 2.39-2.31 (m, 1H), 2.27-2.13 (m, 2H), 2.11-1.97 (m, 3H), 1.32-1.22 (m, 2H), 0.92-0.79 (m, 1H) |
| Example 34 | | |
| 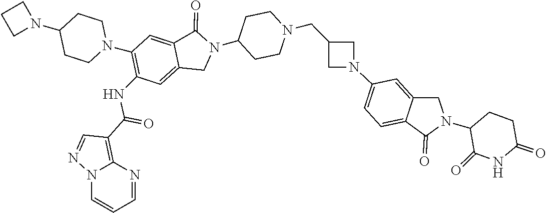 [SMILES: O=C1CCC(N2Cc3cc(N4CC(CN5CCC(N6Cc7cc(NC(=O)c8cnn9cccnc89)c(N8CCC(N9CCC9)CC8)cc7C6=O)CC5)C4)ccc3C2=O)C(=O)N1] | 826.5 | DMSO-d₆ δ = 10.80-11.08 (m, 2 H) 9.41 (d, J = 7.6 Hz, 1 H) 8.94 (d, J = 2.4 Hz, 1 H) 8.76 (d, J = 9.6 Hz, 2 H) 8.21 (s, 1 H) 7.55 (s, 1 H) 7.42- 7.51 (m, 2 H) 6.43-6.54 (m, 2 H) 4.96-5.11 (m, 1H) 4.45 (s, 1 H) 4.26-4.35 (m, 1 H) 4.11- 4.22 (m, 1 H) 3.95-4.08 (m, 3 H) 3.52-3.61 (m,3H)3.14- 3.23 (m, 6 H) 2.91-3.02 (m, 5 H) 2.72-2.77 (m, 1 H) 2.57-2.69 (m, 3 H) 2.38 (m, 1 H) 2.07-2.23 (m,3H) 1.89-2.05 (m, 4 H) 1.74- |
| Example 35 | | 1.87(m, 4 H) 1.52-1.71 (m, 4H) |
| 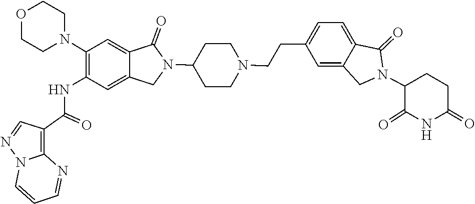 [SMILES: O=C1CCC(N2Cc3cc(CCN4CCC(N5Cc6cc(NC(=O)c7cnn8cccnc78)c(N7CCOCC7)cc6C5=O)CC4)ccc3C2=O)C(=O)N1] | 732.4 | DMSO-d₆ δ = 10.98 (s, 1H), 10.90 (s, 1H), 9.41 (dd, J = 2.0,7.2 Hz, 1H), 9.00 (dd, J = 1.6, 4.0 Hz, 1H), 8.76-8.75 (m, 2H), 8.31 (s, 1H), 7.65 (d, J = 8.0 Hz, 1H), 7.57 (s, 1H), 7.49 (s, 1H), 7.42-7.37 (m, 2H), 5.11 (dd, J = 5.2, 13.2 Hz, 1H), 4.47-4.40 (m, 3H), 4.33-4.27 (m, 1H), 4.04-3.96 (m, 1H), 3.92-3.88 (m, 4H), 3.08- 3.03 (m, 2H), 2.94-2.87 (m,7H), 2.64-2.58 (m, 3H), 2.44-2.36 |
| Example 36 | | (m, 1H), 2.17-2.07 (m, 2H), 2.04- |
| | | 1.96 (m, 1H), 1.87-1.74 (m, 2H), |
| | | 1.73-1.65 (m, 2H) |
| 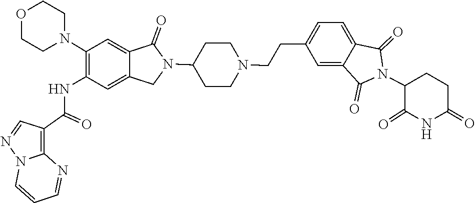 [SMILES: O=C1CCC(N2C(=O)c3ccc(CCN4CCC(N5Cc6cc(NC(=O)c7cnn8cccnc78)c(N7CCOCC7)cc6C5=O)CC4)cc3C2=O)C(=O)N1] | 746.4 | DMSO-d₆ δ = 11.12 (s, 1 H) 10.90 (s, 1 H) 9.41 (dd, J = 1.6, 6.8 Hz, 1 H) 9.00 (dd, J = 1.6, 4.4 Hz, 1 H) 8.75 (s, 2 H) 8.29 (s, 1 H) 7.82-7.88 (m, 2 H) 7.75-7.80 (m, 1 H) 7.57 (s, 1 H) 7.36- 7.42 (m, 1 H) 5.15 (dd, J = 5.2, 12.4 Hz, 1 H) 4.44 (s, 2 H) 3.94- 4.09 (m, 1 H) 3.85-3.93 (m, 4 H) 3.06 (d, J =11.2 Hz, 2 H) 2.98 (m, 2 H) 2.84-2.89 (m, 1H) 2.52-2.69 (m, 5 H) 1.99-2.20 (m, 4 H) 1.64-1.82 (m, 4H) |
| Example 37 | | |
| 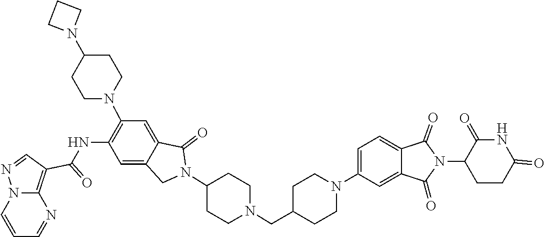 [SMILES: O=C1CCC(N2C(=O)c3ccc(N4CCC(CN5CCC(N6Cc7cc(NC(=O)c8cnn9cccnc89)c(N8CCC(N9CCC9)CC8)cc7C6=O)CC5)CC4)cc3C2=O)C(=O)N1] | 868.3 | DMSO-d₆ δ = 11.16-10.87 (m, 2H), 9.41 (dd, J = 1.6,6.8 Hz, 1H), 8.94 (m, 1H), 8.81-8.67 (m, 2 H), 7.65 (d, J = 8.4 Hz, 1 H), 7.54 (s, 1 H), 7.45 (dd, J = 4.0, 6.8 Hz, 1H), 7.31 (s, 1H), 7.23 (dd, J = 2.0, 8.8 Hz, 1 H), 5.07 (dd, J = 5.2, 12.8 Hz, 1 H), 4.44 (s, 2 H), 4.11-3.97 (m, 3 H), 3.32-3.28 (m, 2H), 3.17 (d, J = 4.8 Hz, 3 H), 3.04-2.81 (m, 8 H), 2.78-2.64 (m, 2 H), 2.56 (m, 2H), 2.17 (m, 2H), 2.02 (m, 5 H), 1.81 (m, 6 H), 1.70-1.58 (m, 4H), 1.25-1.14 (m, 2 H) |
| Example 38 | | |
| 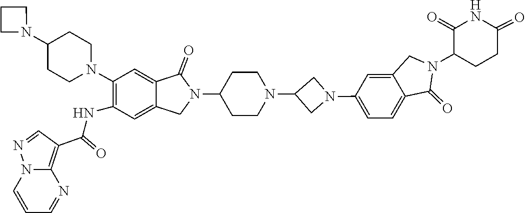 [SMILES: O=C1CCC(N2Cc3cc(N4CC(N5CCC(N6Cc7cc(NC(=O)c8cnn9cccnc89)c(N8CCC(N9CCC9)CC8)cc7C6=O)CC5)C4)ccc3C2=O)C(=O)N1] | 812.3 | DMSO-d₆ δ = 10.96 (d, J = 14 Hz, 2H), 9.41 (dd, J = 1.6, 7.2 Hz, 1H), 8.94 (dd, J = 1.6, 4.34 Hz, 1H), 8.75 (d, J = 8.0 Hz, 2H), 8.22 (s, 1H), 7.55 (s, 1H), 7.50 (d, J = 8.4 Hz, 1H), 7.45 (dd, J = 4.4, 6.8 Hz, 1H), 6.54 (s, 1H), 6.52-6.48 (m, 1H), 5.04 (dd, J = 5.2, 12.8 Hz, 1H), 4.44 (s, 2H), 4.36- 4.27 (m, 1H), 4.22-4.14 (m, 1H), 4.04 (m, 2H), 3.73-3.68 (m, 2H), 3.19 (m, 4H), 3.01-2.87 (m, |
| Example 39 | | 6H), 2.77-2.70 (m, 2H), 2.63-2.55 |
| | | (m, 2H), 2.39-2.33 (m, 1H), 2.30- |
| | | 2.22 (m, 1H), 2.08-1.90 (m, 6H), |
| | | 1.85-1.70(m, 6H), 1.67-1.57 (m, |
| | | 2H) |
| 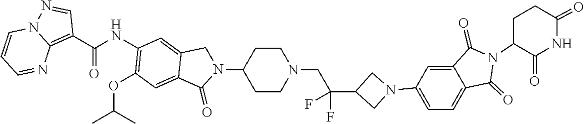 [SMILES: CC(C)Oc1cc2c(cc1NC(=O)c1cnn3cccnc13)CN(C1CCN(CC(F)(F)C3CN(c4ccc5c(c4)C(=O)N(C4CCC(=O)NC4=O)C5=O)C3)CC1)C2=O] | 810.1 | DMSO-d₆ δ = 10.94 (s, 1 H) 10.71 (s, 1 H) 9.37 (br d, J = 6.8 Hz, 1 H) 8.89 (br d, J = 2.8 Hz, 1 H) 8.73 (d, J = 12.0 Hz, 2 H) 7.67 (d, J = 8.4 Hz, 1 H) 7.31- 7.38 (m, 2 H) 6.88 (s, 1 H) 6.73 (br d, J = 8.0 Hz, 1H)5.07 (dd, J = 12.4, 5.2 Hz, 1 H) 4.88 |
| Example 40 | | (quin, J = 6.0 Hz, 1 H) 4.41 (s, |
| | | 2 H) 4.16-4.27 (m, 2 H) 4.08 |
| | | (m, 2H) 3.99 (br t, J = 11.2 Hz, |
| | | 1 H) 3.55 (m, 1 H) 2.99 (br d, |
| | | J = 9.6 Hz, 2 H) 2.84-2.94 (m, |
| | | 3 H) 2.52-2.64 (m, 3 H) 2.40 |
| | | (br t, J = 10.8 Hz, 3 H) 2.00- |
| | | 2.07 (m, 1 H) 1.81 (br s, 2 H) |
| | | 1.68 (m, 2 H) 1.45 (d, J = 6.0 |
| | | Hz, 6 H) |
| 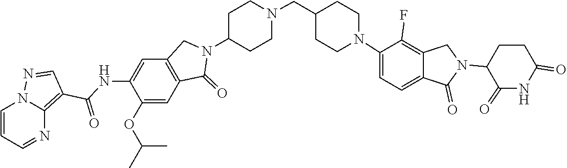 [SMILES: CC(C)Oc1cc2c(cc1NC(=O)c1cnn3cccnc13)CN(C1CCN(CC3CCN(c4ccc5c(c4F)CN(C4CCC(=O)NC4=O)C5=O)CC3)CC1)C2=O] | 792.2 | DMSO-d₆ δ = 10.98 (s, 1 H) 10.75 (s, 1H) 9.39 (dd, J = 7.2, 1.6 Hz, 1 H) 8.88 (dd, J = 4.4, 1.6 Hz, 1H) 8.73 (d, J = 1.6 Hz, 2 H) 8.21 (s, 1 H) 7.46 (d, J = 8.0 Hz, 1 H) 7.31-7.38 (m, 2 H) 7.15 (t, J = 8.0 Hz, 1 H) 5.07 (dd, J = 13.2, 4.8 Hz, 1 H) 4.89 (dt, J = 12.0, 6.0 Hz, 1 H) 4.28-4.51 (m, 5 H) 3.99 (br t, J =11.6 Hz, 1 H) 2.85-2.99 |
| Example 41 | | (m, 4H) 2.78 (br t, J = 11.2 Hz, |
| | | 2 H) 2.57-2.67 (m, 3 H) 2.42 |
| | | (br dd, J =13.2, 4.8 Hz, 1 H) |
| | | 2.21 (brd, J = 6.8Hz, 2H) 1.97- |
| | | 2.05(m, 3 H) 1.78-1.85 (m, |
| | | 3 H) 1.70 (br s, 3 H) 1.44 (d, J = |
| | | 6.0 Hz, 6H) 1.23-1.33 (m, 2H) |
| 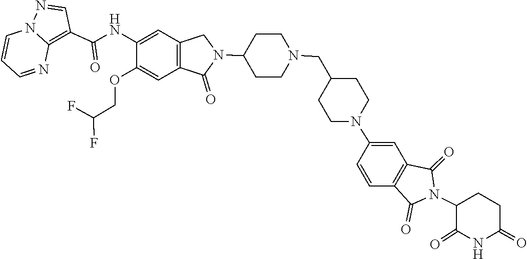 [SMILES: O=C1CCC(N2C(=O)c3ccc(N4CCC(CN5CCC(N6Cc7cc(NC(=O)c8cnn9cccnc89)c(OCC(F)F)cc7C6=O)CC5)CC4)cc3C2=O)C(=O)N1] | 810.1 | DMSO-d₆ δ = 11.08 (s, 1H), 10.78 (s, 1H), 9.39 (dd, J = 1.6, 6.8 Hz, 1H), 8.86 (dd, J = 1.6, 4.4 Hz, 1H), 8.75 (d, J = 7.2 Hz, 2H), 7.65 (d, J = 8.8 Hz, 1H), 7.41 (s, 1H), 7.40- 7.35 (m, 1H), 7.30 (s, 1H), 7.23 (dd, J = 2.0, 8.4 Hz, 1H), 6.77-6.42 (m, 1H), 5.06 (dd, J = 5.2, 12.8 Hz, 1H), 4.66- 4.52 (m, 2H), 4.45 (s, 2H), 4.10-3.93 (m, 3H), 3.02-2.82 (m, 6H), 2.63-2.57 (m, 1H), 2.18 (d, J = 6.4 Hz, 2H), 2.06-1.97 (m, 3H), 1.89-1.74 (m, 5H), 1.74-1.65 (m,2H), 1.23-1.08 (m, 2H) |
| Example 42 | | |
| 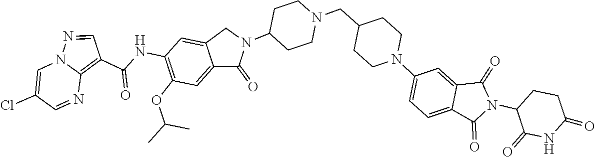 [SMILES: CC(C)Oc1cc2c(cc1NC(=O)c1cnn3cc(Cl)cnc13)CN(C1CCN(CC3CCN(c4ccc5c(c4)C(=O)N(C4CCC(=O)NC4=O)C5=O)CC3)CC1)C2=O] | 822.2 | DMSO-d₆ δ = 11.08 (s, 1 H) 10.55 (s, 1 H) 9.87 (s, 1 H) 8.94 (s, 1 H) 8.77 (s, 1 H) 8.72 (s, 1 H) 7.67 (d, J = 8.40 Hz, 1 H) 7.33 (s, 1 H) 7.31 (s, 1 H) 7.25 (d, J = 8.4 Hz, 1 H) 5.09- 5.05 (m, 1 H) 4.91-4.88 (m, 1 H) 4.43 (s, 2 H) 4.07-3.99 (m, 3 H) 2.97-2.94 (m, 6 H) 2.53- 2.52 (m, 1 H) 2.17-2.15 (m, 2 |
| Example 43 | | H) 2.02-2.0 (m, 3H) 1.83-1.79 |
| | | (m, 7H) 1.44 (d, J = 6.00 Hz, 6 |
| | | H) 1.18-1.15 (m, 2H) |
| 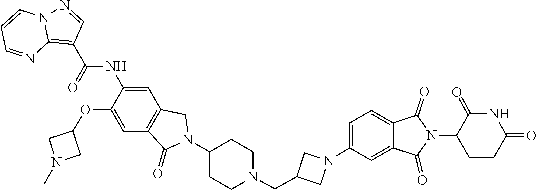 [SMILES: CN1CC(Oc2cc3c(cc2NC(=O)c2cnn4cccnc24)CN(C2CCN(CC4CN(c5ccc6c(c5)C(=O)N(C5CCC(=O)NC5=O)C6=O)C4)CC2)C3=O)C1] | 787.1 | DMSO-d₆ δ 11.27-10.85 (m, 1H), 10.75 (s, 1H), 9.41 (dd, J = 1.6, 7.2 Hz, 1H), 8.92 (dd, J = 1.6, 4.4 Hz, 1H), 8.75 (d, J = 1.2 Hz, 2H), 8.24 (s, 1H), 7.63 (d, J = 8.4 Hz, 1H), 7.38 (dd, J = 4.0, 6.8 Hz, 1H), 7.04 (s, 1H), 6.77 (d, J = 2.0 Hz, 1H), 6.64 (dd, J = 2.0, 8.4 Hz, 1H), 5.11-5.02 (m, 2H), 4.43 (s, 2H), 4.14 (br t, J = 7.6 Hz, 2H), 4.05-3.93 (m, 1H), 3.90- |
| Example 44 | | 3.82 (m, 2H), 3.74-3.66 (m, |
| | | 2H), 3.25-3.21 (m, 2H), 3.07- |
| | | 2.82 (m, 5H), 2.66-2.59 (m, |
| | | 2H), 2.36 (s, 3H), 2.15-2.06 |
| | | (m, 2H), 2.05-1.98 (m, 1H), |
| | | 1.85-1.73 (m,2H), 1.69 (br d, |
| | | J = 8.8 Hz, 2H) |
| 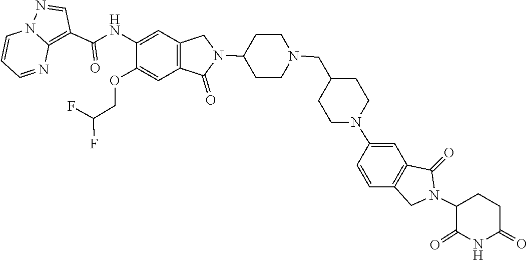 [SMILES: O=C1CCC(N2Cc3ccc(N4CCC(CN5CCC(N6Cc7cc(NC(=O)c8cnn9cccnc89)c(OCC(F)F)cc7C6=O)CC5)CC4)cc3C2=O)C(=O)N1] | 796.4 | DMSO-d₆ δ 10.97 (s, 1 H) 10.79 (s, 1 H) 9.41 (dd, J = 6.80, 1.44 Hz, 1 H) 8.84- 8.90 (m, 1 H) 8.76 (d, J = 6.80 Hz, 2 H) 7.43 (s, 1 H) 7.35- 7.42 (m, 2 H) 7.26 (dd, J = 8.40, 1.63 Hz, 1 H) 7.16 (d, J = 2.00 Hz, 1 H) 6.43-6.77 (m, 1 H) 5.10 (dd, J = 13.20, 5.20 Hz, 1 H) 4.60 (td, J = 14.40, 3.20 Hz, 2 H) 4.46 (s, 2 H) 4.28-4.38 (m, 1 H) 4.15-4.24 (m, 1 H) 3.93-4.06 (m, 1H) 3.76 (br d, J = 11.20 Hz, 2 H) 2.84-3.01 (m, 4 H) 2.72 (br t, J = 11.20 Hz, 3 H) 2.20 (br d, J = 6.80 Hz, |
| Example 45 | | 2 H) 2.00 (br d, J = 10.80 Hz, 3 H) |
| | | 1.82 (br d, J = 11.60 Hz, 4 H) 1.65- |
| | | 1.75 (m, 3 H) 1.17-1.28 (m, 2H) |
| 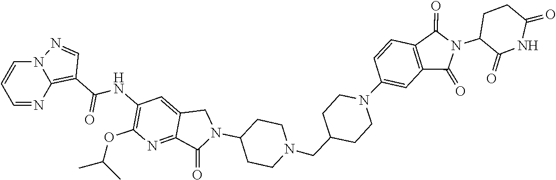 [SMILES: CC(C)Oc1nc2c(cc1NC(=O)c1cnn3cccnc13)CN(C1CCN(CC3CCN(c4ccc5c(c4)C(=O)N(C4CCC(=O)NC4=O)C5=O)CC3)CC1)C2=O] | 789.3 | DMSO-d₆ δ 10.97 (s, 1 H) 10.79 (s, 1 H) 9.41 (dd, J = 6.80, 1.44 Hz, 1 H) 8.84-8.90 (m, 1 H) 8.76 (d, J = 6.80 Hz, 2 H) 7.43 (s, 1 H) 7.35-7.42 (m, 2 H) 7.26 (dd, J = 8.40, 1.63 Hz, 1 H) 7.16 (d, J = 2.00 Hz, 1 H) 6.43-6.77 (m, 1 H) 5.10 (dd, J = 13.20, 5.20 Hz, 1 H) 4.60 (td, J = 14.40, 3.20 Hz, 2 H) 4.46 (s, 2 H) 4.28-4.38 (m, 1 H) 4.15-4.24 (m, 1 H) 3.93-4.06 (m, 1H) 3.76 (br d, J =11.20 Hz, 2 H) |
| Example 46 | | 2.84-3.01 (m, 4 H) 2.72 (br t, J = |
| | | 11.20 Hz, 3 H) 2.20 (br d, J = 6.80 |
| | | Hz, 2 H) 2.00 (br d, J = 10.80 Hz, 3 |
| | | H) 1.82 (br d, J = 11.60 Hz, 4 H) |
| | | 1.65-1.75 (m, 3 H) 1.17-1.28 (m, |
| | | 2H) |
| 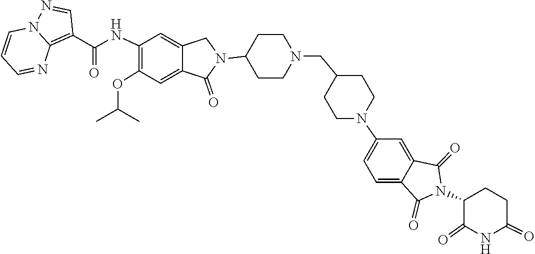 [SMILES: CC(C)Oc1cc2c(cc1NC(=O)c1cnn3cccnc13)CN(C1CCN(CC3CCN(c4ccc5c(c4)C(=O)N([C@@H]4CCC(=O)NC4=O)C5=O)CC3)CC1)C2=O] | 788.3 | DMSO-d₆ δ 11.09 (s, 1 H) 10.72 (s, 1 H) 9.36 (d, J = 6.8 Hz, 1 H) 8.86 (d, J = 3.2 Hz, 1 H) 8.72 (d, J = 4.0 Hz, 2 H) 7.63 (d, J = 8.4 Hz, 1 H) 7.30- 7.36 (m, 2 H) 7.25-7.30 (m, 1 H) 7.20 (br d, J = 8.4 Hz, 1 H) 5.02-5.11 (m, 1 H) 4.88 (s, 1 H) 4.40 (s, 2 H) 4.02 (m, 3 H) 2.87-2.98 (m, 5 H) 2.52-2.65 (m, 2 H) 2.15 (br d, J = 5.6 Hz, 2H) 1.95-2.04 (m, 3 H) 1.78 (br d, J = 11.2 Hz, 5 H) 1.63- 1.70 (m, 2 H) 1.43 (d, J = 6.0 Hz, 6H) 1.08-1.18 (m, 2 H) |
| Example 47 | | |
| 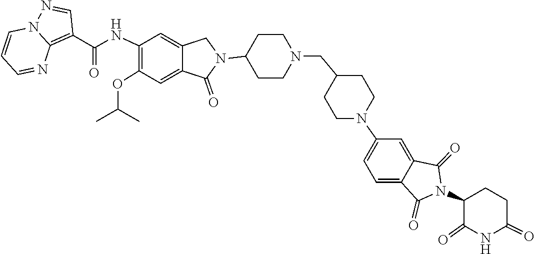 [SMILES: CC(C)Oc1cc2c(cc1NC(=O)c1cnn3cccnc13)CN(C1CCN(CC3CCN(c4ccc5c(c4)C(=O)N([C@H]4CCC(=O)NC4=O)C5=O)CC3)CC1)C2=O] | 788.3 | DMSO-d₆ δ 11.09 (s, 1 H) 10.71 (s, 1 H) 9.35 (d, J = 6.8 Hz, 1 H) 8.84-8.88 (m, 1 H) 8.66-8.76 (m, 2 H) 7.62 (d, J = 8.4 Hz, 1 H) 7.29-7.36 (m, 2 H) 7.27 (s, 1 H) 7.18 (br d, J = 8.0 Hz, 1 H) 5.07 (dd, J = 12.8, 5.2 Hz, 1 H) 4.82- 4.93 (m, 1 H) 4.40 (s, 2 H) 4.01 (m, 3 H) 2.80-3.01 (m, 5 H) 2.51-2.64 (m, 2 H) 2.14 (br d, J = 3.2 Hz, 2 H) 1.92- 2.07 (m, 3 H) 1.78 (br d, J = 10.8 Hz, 5 H) 1.67 (br d, J = 8.8 Hz, 2 H) 1.43 (d, J = 6.0 Hz, 6H) 1.06-1.19 (m, 2 H) |
| Example 48 | | |
| 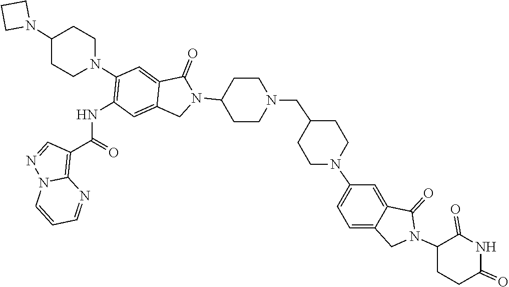 [SMILES: O=C1CCC(N2Cc3ccc(N4CCC(CN5CCC(N6Cc7cc(NC(=O)c8cnn9cccnc89)c(N8CCC(N9CCC9)CC8)cc7C6=O)CC5)CC4)cc3C2=O)C(=O)N1] | 854.3 | DMSO-d₆ δ 10.98 (br d, J = 1.20 Hz, 1 H) 9.42 (d, J = 6.80 Hz, 1 H) 8.91-8.97 (m, 1 H) 8.76 (d, J = 7.60 Hz, 2 H) 8.18 (d, J = 3.20 Hz, 1 H) 7.55 (s, 1 H) 7.46 (br dd, J = 6.40, 5.20 Hz, 1 H)7.41 (d, J = 8.40 Hz, 1 H) 7.23-7.28 (m, 1H) 7.16 (d, J = 2.40 Hz, 1 H) 5.10 (dd, J = 13.60, 5.20 Hz, 1 H) 4.45 (s, 2 H) 4.29- 4.37 (m, 1 H) 4.15-4.25 (m, 1 H) 3.91-4.06 (m, 1H) 3.72-3.81 (m, 2 H) 3.14-3.25 (m, 6 H) 2.96 (br dd, J = 7.20, 1.20 Hz, 4 H) 2.70-2.76 (m, 3 H) 2.20 (br d, J = 7.60 Hz, 2 H) 1.96-2.06 (m, 6 H) 1.78-1.86 (m, 6 H) 1.56-1.73 (m, 6 H) 1.11-1.34 (m, 3 H) |
| Example 49 | | |
| 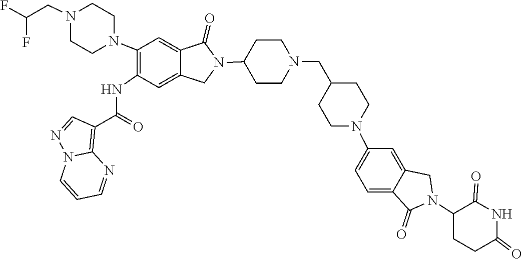 [SMILES: O=C1CCC(N2Cc3cc(N4CCC(CN5CCC(N6Cc7cc(NC(=O)c8cnn9cccnc89)c(N8CCN(CC(F)F)CC8)cc7C6=O)CC5)CC4)ccc3C2=O)C(=O)N1] | 864.3 | DMSO-d₆ δ 10.96 (s, 1H), 10.83 (s, 1H), 9.40 (dd, J = 1.2, 7.8 Hz, 1H), 9.00 (dd, J = 1.2, 4.0 Hz, 1H), 8.81-8.69 (m, 2H), 8.16 (s, 1H), 7.59-7.45 (m, 2H),7.40 (dd, J = 4.0, 6.8 Hz, 1H), 7.10-6.99 (m, 2H), 6.42-6.05 (m, 1H), 5.05 (dd, J = 4.8,13.2 Hz, 1H), 4.45 (s, 2H), 4.37-4.28 (m, 1H), 4.24-4.16 (m, 1H), 4.04-3.95 (m, 1H), 3.88 (d, J = 12.4 Hz, 2H), 2.95-2.80 (m, 15H), 2.63-2.54 (m, 1H), 2.39-2.35 (m, 1H), 2.23-2.15 (m, 2H), 2.09-1.95 (m, 3H), 1.85-1.68(m, 7H), 1.26-1.12 (m, 2H) |
| Example 50 | | |
| 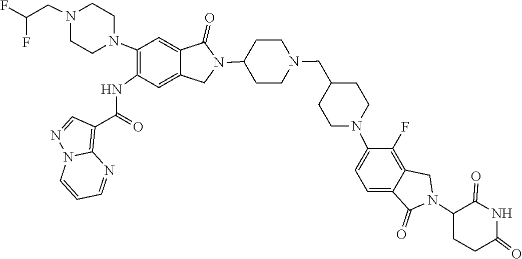 [SMILES: O=C1CCC(N2Cc3c(ccc(N4CCC(CN5CCC(N6Cc7cc(NC(=O)c8cnn9cccnc89)c(N8CCN(CC(F)F)CC8)cc7C6=O)CC5)CC4)c3F)C2=O)C(=O)N1] | 882.3 | DMSO-d₆ δ 10.99 (s, 1H), 10.83 (s, 1H), 9.40 (dd, J = 1.6, 6.8 Hz, 1H), 9.00 (dd, J = 1.2, 4.0 Hz, 1H), 8.81-8.69 (m, 2H), 7.54 (s, 1H), 7.47 (d, J = 8.0 Hz, 1H), 7.43-7.36 (m, 1H), 7.16 (t, J = 8.0 Hz, 1H), 6.43-6.03 (m, 1H), 5.08 (dd, J = 4.8,13.2 Hz, 1H), 4.50-4.41 (m, 3H), 4.36-4.25 (m, 1H), 4.07-3.93 (m, 1H), 3.52- 3.48 (m,2H), 2.97-2.79 (m, 15H), 2.64-2.55 (m, 1H), 2.45-2.40 (m, 1H), 2.22 (d, J = 6.4 Hz, 2H), 2.09-1.96 (m, 3H), 1.86-1.76 (m, 4H), 1.73-1.62 (m, 3H), 1.34-1.24 (m, 2H) |
| Example 51 | | |
| 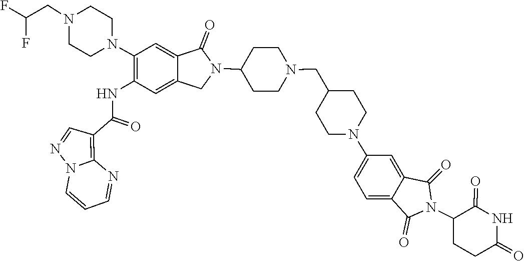 [SMILES: O=C1CCC(N2C(=O)c3ccc(N4CCC(CN5CCC(N6Cc7cc(NC(=O)c8cnn9cccnc89)c(N8CCN(CC(F)F)CC8)cc7C6=O)CC5)CC4)cc3C2=O)C(=O)N1] | 878.3 | DMSO-d₆ δ 11.09 (s, 1H), 10.83 (s, 1H), 9.40 (dd, J = 1.6, 6.8 Hz, 1H), 9.00 (dd, J = 1.6, 4.0 Hz, 1H), 8.75 (d, J = 3.2 Hz, 2H),8.15(s, 1H), 7.65 (d, J = 8.4 Hz, 1H), 7.54 (s, 1H), 7.39 (dd, J = 4.4, 6.8 Hz, 1H), 7.31 (s, 1H),7.23 (dd, J = 2.0, 8.8 Hz, 1H), 6.42 6.01 (m, 1H), 5.07 (dd, J = 5.2, 12.8 Hz, 1H), 4.45 (s, 2H), 4.09-3.97 (m, 3H), 2.99-2.83 (m, 15H), 2.62-2.56 (m, 1H), 2.46-2.39 (m, 1H), 2.19 (d, J = 6.0 Hz, 2H), 2.10-1.96 (m, 3H), 1.88-1.75 (m, 5H), 1.72-1.62(m, 2H), 1.25-1.08 (m, 2H) |
| Example 52 | | |
| 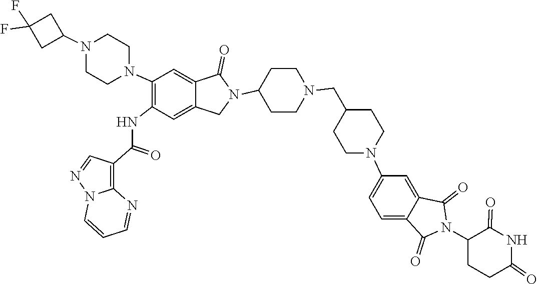 [SMILES: O=C1CCC(N2C(=O)c3ccc(N4CCC(CN5CCC(N6Cc7cc(NC(=O)c8cnn9cccnc89)c(N8CCN(C9CC(F)(F)C9)CC8)cc7C6=O)CC5)CC4)cc3C2=O)C(=O)N1] | 904.3 | CDCl3 δ 10.81 (s, 1 H), 8.88 (dd, J = 1.6,7.2 Hz, 1H), 8.84 (d, J = 4.8 Hz, 2 H), 8.78 (dd, J = 1.6, 4.0 Hz, 1 H), 8.33-8.29 (m, 1 H), 8.12 (s, 1 H), 7.73 (s, 1 H), 7.69 (d, J = 8.8 Hz, 1 H), 7.29 (d, J = 2.0 Hz, 1H),7.15 (dd, J = 4.0, 7.2 Hz, 1 H), 7.06 (dd, J = 2.0, 8.4 Hz, 1 H), 4.95 (dd, J = 4.8, 12.0 Hz, 1H), 4.36-4.26 (m, 1H), 3.98 (m, 2 H), 3.25-3.12 (m, 2 H), 3.08-2.98 (m, 6 H), 2.97-2.84 (m, 3 H), 2.84-2.63 (m, 9 H), 2.60-2.45 (m, 3 H), 2.41 (m, 2 H), 2.36-2.25 (m, 2 H), 2.19-2.11 (m, 1 H), 1.95 (m, 3 H), 1.88 (m, 3 H), 1.41-1.28 (m, 2 H) |
| Example 53 | | |
| 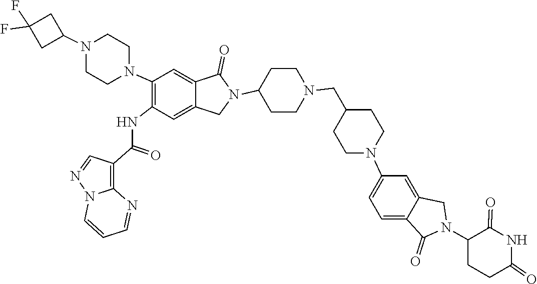 [SMILES: O=C1CCC(N2Cc3cc(N4CCC(CN5CCC(N6Cc7cc(NC(=O)c8cnn9cccnc89)c(N8CCN(C9CC(F)(F)C9)CC8)cc7C6=O)CC5)CC4)ccc3C2=O)C(=O)N1] | 890.3 | DMSO-d₆ δ 10.95 (s, 1 H), 10.83 (s, 1 H), 9.40 (dd, J = 1.6, 7.2 Hz, lH),8.98(dd, J = 1.6, 4.0 Hz, 1 H), 8.75 (d, J = 2.8 Hz, 2H), 7.55-7.47 (m, 2 H), 7.43-7.37 (m, 1 H), 7.09- 7.02 (m, 2 H), 5.04 (dd, J = 4.8, 12.8 Hz, 1 H), 4.45 (s, 2 H), 4.36-4.27 (m, 1 H), 4.23- 4.15 (m, 1H), 4.04-3.93 (m, 1H),3.88 (m, 2 H), 2.99-2.87 (m, 7 H), 2.87-2.67 (m, 6 H), 2.67-2.54 (m, 4 H), 2.41-2.30 (m, 3 H), 2.20 (m, 2 H), 2.06-1.93 (m, 3 H), 1.80 (m, 5H), 1.72-1.63 (m,2H), 1.24-1.13 (m, 2H) |
| Example 54 | | |
| 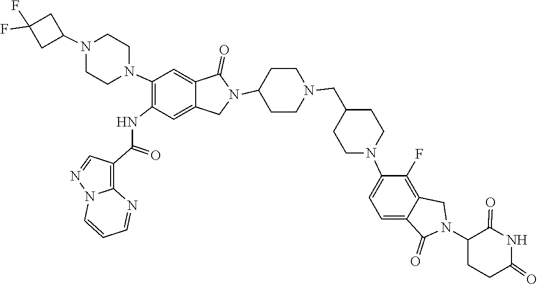 [SMILES: O=C1CCC(N2Cc3c(ccc(N4CCC(CN5CCC(N6Cc7cc(NC(=O)c8cnn9cccnc89)c(N8CCN(C9CC(F)(F)C9)CC8)cc7C6=O)CC5)CC4)c3F)C2=O)C(=O)N1] | 908.3 | DMSO-d₆ δ 10.97 (s, 1 H), 10.83 (s, 1 H), 9.40 (d, J = 6.4 Hz, 1 H), 9.02-8.94 (m, 1 H), 8.75 (d, J = 4.0 Hz, 2H), 8.16 (s, 1 H), 7.53 (s, 1 H), 7.47 (d, J = 7.6 Hz, 1H),7.40 (m, 1 H), 7.17 (m, 1 H), 5.07 (m, 1 H), 4.56-4.25 (m, 5 H), 4.07- 3.92 (m, 1 H), 3.53-3.46 (m, 2 H), 3.00-2.85 (m, 9H),2.83-2.73 (m, 4 H), 2.67-2.60 (m, 4 H), 2.23 (m, 2 H), 2.08-1.95 (m, 4H), 1.87-1.78 (m, 4 H), 1.75-1.62 (m, 4 H), 1.27 (m, 3 H) |
| Example 55 | | |
| 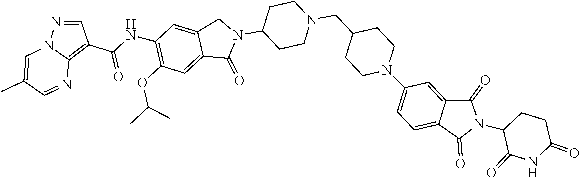 [SMILES: Cc1cnc2c(C(=O)Nc3cc4c(cc3OC(C)C)C(=O)N(C3CCN(CC5CCN(c6ccc7c(c6)C(=O)N(C6CCC(=O)NC6=O)C7=O)CC5)CC3)C4)cnn2c1] | 802.3 | DMSO-d₆ δ = 11.09 (s, 1H), 10.69 (s, 1H), 9.25 (s, 1H), 8.79 (d, J = 1.4 Hz, 1H), 8.73 (s, 1H), 8.64 (s, 1H), 7.64 (d, J = 8.4 Hz, 1H), 7.33 (s, 1H), 7.30 (s, 1H),7.22 (br d, J = 8.4 Hz, 1H), 5.07 (dd, J = 5.4, 12.6 Hz, 1H), 4.93-4.85 (m, 1H), 4.42 (s, 2H), 4.07-3.94 (m, 3H), 3.00-2.83 (m, 5H), 2.63- 2.52 (m, 3H), 2.43 (s, 3H), |
| Example 56 | | 2.17 (br d, J = 5.6 Hz, 2H), |
| | | 2.06-1.95 (m, 3H), 1.84- |
| | | 1.73 (m, 5H), 1.68 (br d, J = |
| | | 8.0 Hz, 2H), 1.44 (d, J = 6.0 |
| | | Hz, 4H), 1.50-1.38 (m, 1H), |
| | | 1.15 (br d, J = 11.2 Hz, 2H) |

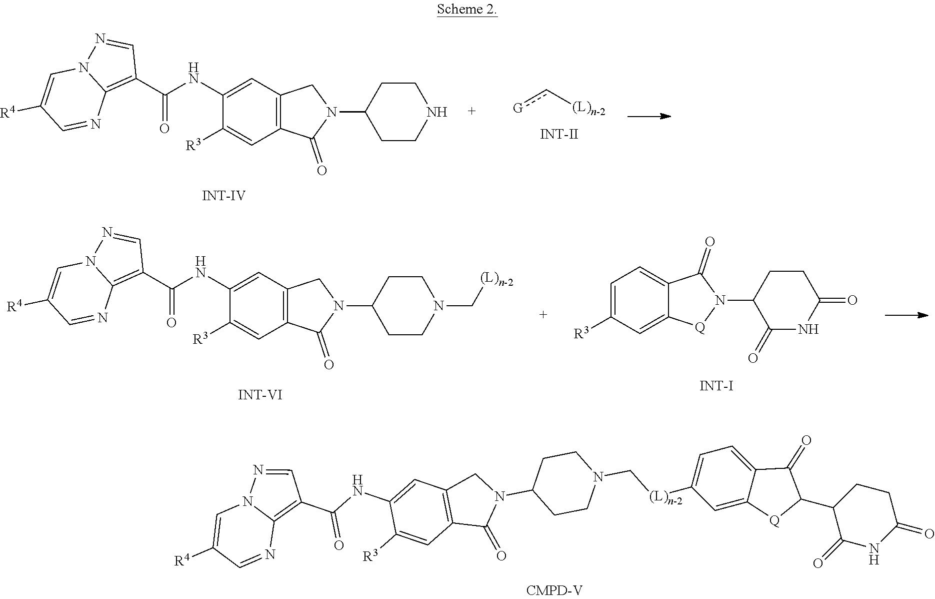

A compound of formula INT-IV, wherein R³ is an ether, alkyl group or an amine and R⁴ is optionally H, an amine, methyl, or chloro group, may be reacted with a reagent INT-II (commercially available or readily prepared using standard reaction techniques known to one skilled in the art) under reductive amination or alkylation conditions to produce a compound of formula INT-VI, wherein L and n are as defined herein. Compounds of formula INT-VI can be furnished from N-alkylation where G is an appropriate leaving group (e.g., OMs, OTs, Cl, etc.) or through reductive amination where G is an aldehyde or ketone. When G is a leaving group, suitable reaction conditions are those for an alkylation reaction, e.g., diisopropylethylamine, potassium iodide, DMSO or acetonitrile, 80° C. When G is a carbonyl, suitable reaction conditions are those for a reductive amination reaction, e.g., sodium cyanoborohydride, methanol, dichloromethane, acetic acid, room temperature. Compounds of formula INT-VI can then be reacted with compounds of formula INT-I wherein Q is CH₂ or C═O and X^{S1} is an alcohol or a halide to produce compound of formula CMPD-V.

### Preparation of Example 57: N-(2-(1-((1-(2-(2,6-dioxopiperidin-3-yl)-1,3-dioxoisoindolin-5-yl)-3-fluoroazetidin-3-yl)methyl)piperidin-4-yl)-6-isopropoxy-1-oxoisoindolin-5-yl)pyrazolo[1,5-a]pyrimidine-3-carboxamide

### Step 1:

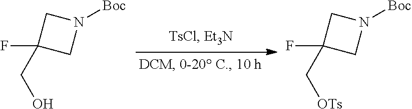 [SMILES: CC(C)(C)OC(=O)N1CC(F)(CO)C1 ; Cc1ccc(S(=O)(=O)OCC2(F)CN(C(=O)OC(C)(C)C)C2)cc1]

To a mixture of tert-butyl 3-fluoro-3-(hydroxymethyl)azetidine-1-carboxylate (5.0 g, 24.4 mmol, 1 eq), triethylamine (7.40 g, 73.09 mmol, 10.17 mL, 3 eq) in dichloromethane (150 mL) was added 4-methylbenzene-1-sulfonyl chloride (7.50 g, 39.34 mmol, 1.61 eq) at 0° C. The reaction mixture was allowed to stir at 20° C. for 10 h under an atmosphere of nitrogen. The mixture was washed with brine (50 mL×3), dried over anhydrous sodium sulfate, concentrated in vacuum to give a residue. The residue was purified by silica gel column chromatography (eluted with petroleum ether/ethyl acetate=10/1 to 3/1) to afford tert-butyl 3-fluoro-3-((tosyloxy)methyl)azetidine-1-carboxylate (8.0 g, 22.3 mmol, 91% yield) as a white solid. ¹H NMR (400 MHz, CDCl₃) δ=7.81 (d, J=8.0 Hz, 2H), 7.38 (d, J=8.4 Hz, 2H), 4.31-4.21 (m, 2H), 4.10-3.89 (m, 4H), 2.47 (s, 3H), 1.44 (s, 9H).

### Step 2:

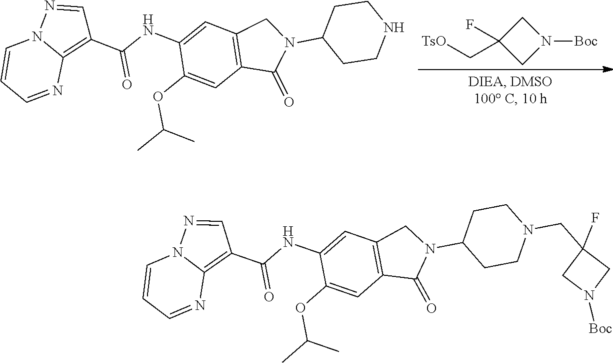 [SMILES: CC(C)Oc1cc2c(cc1NC(=O)c1cnn3cccnc13)CN(C1CCNCC1)C2=O ; Cc1ccc(S(=O)(=O)OCC2(F)CN(C(=O)OC(C)(C)C)C2)cc1 ; CC(C)Oc1cc2c(cc1NC(=O)c1cnn3cccnc13)CN(C1CCN(CC3(F)CN(C(=O)OC(C)(C)C)C3)CC1)C2=O]

To a solution of N-(6-isopropoxy-1-oxo-2-(piperidin-4-yl)isoindolin-5-yl)pyrazolo[1,5-a]pyrimidine-3-carboxamide (100 mg, 230.2 mmol, 1 eq) in dimethyl sulfoxide (2 mL) was added N,N-diisopropylethylamine (148.7 mg, 1.15 mmol, 200.4 mL, 5 eq) and tert-butyl 3-fluoro-3-((tosyloxy)methyl)azetidine-1-carboxylate (165.4 mg, 460.3 mmol, 2 eq). The mixture was stirred at 100° C. for 12 h. To the reaction mixture was added water (10 mL) and the mixture was extracted with ethyl acetate (20 mL×3). The combined organic phase was washed with brine (20 mL), dried over anhydrous sodium sulfate, filtered, and concentrated in vacuum. Crude tert-butyl 3-fluoro-3-((4-(6-isopropoxy-1-oxo-5-(pyrazolo[1,5-a]pyrimidine-3-carboxamido)isoindolin-2-yl)piperidin-1-yl)methyl)azetidine-1-carboxylate (140 mg) was obtained as a white solid and used in subsequent reactions without further purification. LCMS (ESI) m/z: 622.3 [M+H]⁺.

### Step 3:

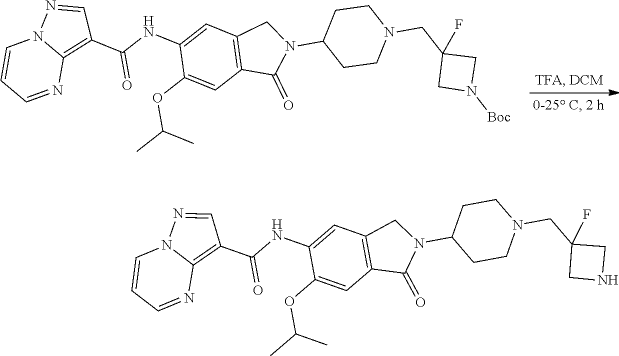 [SMILES: CC(C)Oc1cc2c(cc1NC(=O)c1cnn3cccnc13)CN(C1CCN(CC3(F)CN(C(=O)OC(C)(C)C)C3)CC1)C2=O ; CC(C)Oc1cc2c(cc1NC(=O)c1cnn3cccnc13)CN(C1CCN(CC3(F)CNC3)CC1)C2=O]

To a solution of tert-butyl 3-fluoro-3-((4-(6-isopropoxy-1-oxo-5-(pyrazolo[1,5-a]pyrimidine-3-carboxamido)isoindolin-2-yl)piperidin-1-yl)methyl)azetidine-1-carboxylate (65 mg, 104.5 mmol, 1 eq) in dichloromethane (3 mL) was added trifluoroacetic acid (4.62 g, 40.5 mmol, 3 mL, 387.54 eq). The mixture was stirred at 25° C. for 1 h. The reaction mixture was concentrated under reduced pressure. The residue was diluted with a saturated solution of sodium carbonate (50 mL) and extracted with ethyl acetate (50 mL×4). The combined organic layers were washed with brine (50 mL×2), dried over anhydrous sodium sulfate, filtered, and concentrated under reduced pressure to afford N-(2-(1-((3-fluoroazetidin-3-yl)methyl)piperidin-4-myl)-6-isopropoxy-1-oxoisoindolin-5-yl)pyrazolo[1,5-a]pyrimidine-3-carboxamide (50 mg, 95.9 mmol, 92% yield) as a white solid which was used in the next step without further purification. MS (ESI) m/z: 522.1 [M+H]⁺.

### Step 4:

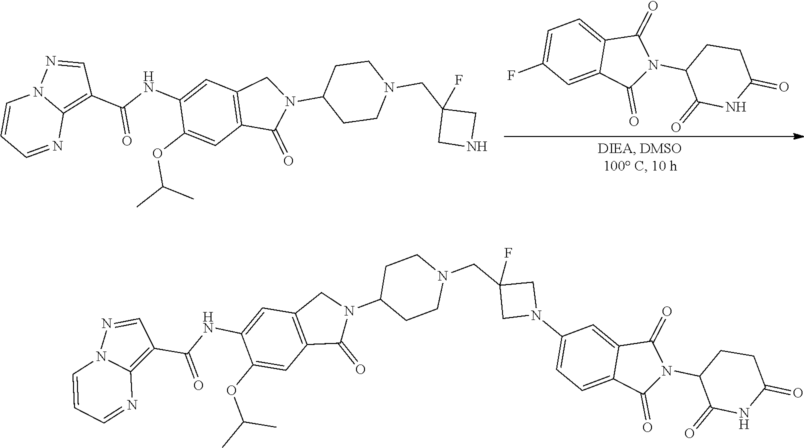 [SMILES: CC(C)Oc1cc2c(cc1NC(=O)c1cnn3cccnc13)CN(C1CCN(CC3(F)CNC3)CC1)C2=O ; O=C1CCC(N2C(=O)c3ccc(F)cc3C2=O)C(=O)N1 ; CC(C)Oc1cc2c(cc1NC(=O)c1cnn3cccnc13)CN(C1CCN(CC3(F)CN(c4ccc5c(c4)C(=O)N(C4CCC(=O)NC4=O)C5=O)C3)CC1)C2=O]

To a solution of N-(2-(1-((3-fluoroazetidin-3-yl)methyl)piperidin-4-yl)-6-isopropoxy-1-oxoisoindolin-5-yl)pyrazolo[1,5-a]pyrimidine-3-carboxamide (60 mg, 115 mmol, 1 eq) in dimethyl sulfoxide (1 mL) was added N,N-diisopropylethylamine (44.6 mg, 345.1 mmol, 60.1 mL, 3 eq) and 2-(2,6-dioxo-3-piperidyl)-5-fluoro-isoindoline-1,3-dione (60.0 mg, 217.2 mmol, 1.89 eq). The mixture was stirred at 100° C. for 10 h. The reaction mixture was allowed to cool to room temperature and quenched by the addition of water (20 mL) at 0° C. The mixture was then diluted with water 10 mL and extracted with ethyl acetate (50 mL×2). The combined organic layers were washed with brine (15 mL×2), dried over anhydrous sodium sulfate, filtered, and concentrated under reduced pressure to give a residue. The residue was purified by preparative HPLC (column: Unisil 3-100 C18 Ultra 150×50 mm×3 mm; mobile phase: [water(0.225% formic acid)-acetonitrile]; B %: 18%-48%, 10 min) to afford N-(2-(1-((1-(2-(2,6-dioxopiperidin-3-yl)-1,3-dioxoisoindolin-5-yl)-3-fluoroazetidin-3-yl)methyl)piperidin-4-yl)-6-isopropoxy-1-oxoisoindolin-5-yl)pyrazolo[1,5-a]pyrimidine-3-carboxamide (8.1 mg, 9.79 mmol, 90 yield, 94) purity) as a white solid. MS (ESI) m/z: 778.3 [M+H]⁺. 1H NMVR (400 MHz, DMSO-d₆) δ=11.07 (br s, 1H), 10.76 (s, 1H), 9.41 (dd, J=1.6, 7.0 Hz, 1H), 8.90 (dd, J=1.6, 4.4 Hz, 1H), 8.74 (s, 2H), 7.69 (d, J=8.0 Hz, 1H), 7.41-7.30 (m, 2H), 6.92 (d, J=2.0 Hz, 1H), 6.78 (dd, J=2.4, 8.4 Hz, 1H), 5.08 (dd, J=5.2, 13.0 Hz, 1H), 4.95-4.83 (m, 1H), 4.41 (s, 2H), 4.31-4.10 (m, 4H), 3.99 (s, 1H), 3.03 (br d, J=12.0 Hz, 2H), 2.96-2.85 (m, 3H), 2.70-2.65 (m, 1H), 2.39-2.27 (m, 4H), 2.09-1.98 (m, 1H), 1.81 (br d, J=8.8 Hz, 2H), 1.68 (br d, J=10.0 Hz, 2H), 1.44 (d, J=6.0 Hz, 6H).

TABLE 2
The following compounds may be prepared in an analogous fashion as described for the
synthesis of Example 57.

| | | ¹H NMR |
|---|---|---|
| | | All spectra acquired at 400 |
| | | MHz unless otherwise |
| Example | MS | indicated |
| Example 57 | See Above | |
| 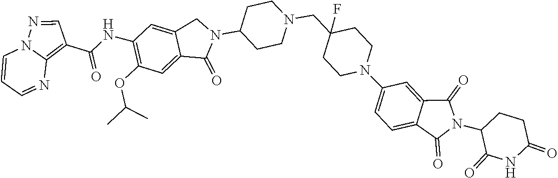 [SMILES: CC(C)Oc1cc2c(cc1NC(=O)c1cnn3cccnc13)CN(C1CCN(CC3(F)CCN(c4ccc5c(c4)C(=O)N(C4CCC(=O)NC4=O)C5=O)CC3)CC1)C2=O] | 806.2 | CDCl₃ δ = 10.71 (s, 1 H), 8.89 (s, 1 H), 8.83-8.87 (m, 2 H), 8.73 (d, J = 2.4 Hz, 1 H), 8.58 (s, 1 H), 7.72 (d, J = 8.4 Hz, 1 H), 7.37 (s, 1 H), 7.34 (s, 1 H), 7.06-7.14 (m, 2 H), 4.97 (dd, J = 12.0, 4.8 Hz, 1H), 4.80 (dt, J = 12.0, 6.0 Hz, 1 H), 4.30 (s, 2 H), 4.23 (s, 1 H), 3.83 (d, J = 14.4 Hz, 2 H), 3.41 (t, J = 12.0 Hz, 2 H), 2.72-3.03 (m, 5 H), |
| Example 58 | | 2.53 (d, J = 19.6 Hz, 2 H), 2.42 |
| | | (s, 2 H), 2.15 (d, J = 5.2 Hz, 1 |
| | | H), 1.94-2.05 (m, 2 H), 1.71-1.90 |
| | | (m, 6 H), 1.51 (d, J = 6.0 Hz, 6 H). |
| 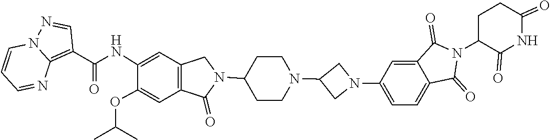 [SMILES: CC(C)Oc1cc2c(cc1NC(=O)c1cnn3cccnc13)CN(C1CCN(C3CN(c4ccc5c(c4)C(=O)N(C4CCC(=O)NC4=O)C5=O)C3)CC1)C2=O] | 746.1 | DMSO-d₆ δ = 11.07 (s, 1 H), 10.75 (s, 1 H), 9.40 (dd, J = 6.8, 1.6 Hz, 1 H), 8.89 (dd, J = 4.4, 1.6 Hz, 1 H), 8.68-8.76 (m, 2 H) 7.66 (d, J = 8.38 Hz, 1 H) 7.31-7.39 (m, 2 H) 6.80 (d, J =1.75 Hz, 1 H) 6.66 (dd, J = 8.4, 2.0 Hz, 1 H), 5.06 (dd, J = 12.8, 5.2 Hz, 1 H), 4.90 (q, J = |
| Example 59 | | 6.0 Hz, 1 H),4.42 (s, 2H),4.13 |
| | | (t, J = 7.6 Hz, 2 H), 4.04 (t, J = |
| | | 11.2 Hz, 1H), 3.86 (dd, J = 8.4, |
| | | 5.2 Hz, 2 H), 3.34-3.40 (m, 1 |
| | | H), 2.95 (d, J = 10.8 Hz, 2 H), |
| | | 2.81-2.90 (m, 1 H), 2.58 (d, J = |
| | | 16.8 Hz, 1 H), 2.54 (s, 2 H), |
| | | 2.02 (d, J = 9.6 Hz, 2 H), 1.82 |
| | | (d, J = 11.2 Hz, 1H), 1.76 (s, 2 |
| | | H), 1.44 (d, J = 6.0 Hz, 6 H), |
| | | 1.23 (d, J = 4.4 Hz, 1 H). |
| 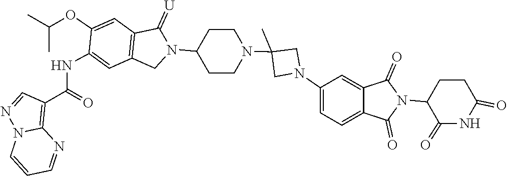 | 760.4 | DMSO-d₆ δ = 11.09 (s, 1H), 10.76 (s, 1H),9.41(dd, J = 1.6, 7.2 Hz, 1H), 8.90 (dd, J = 1.6, 4.2 Hz, 1H), 8.76-8.71 (m, 2H), 8.24 (s, 1H), 7.67 (d, J = 8.4 Hz, 1H), 7.39-7.32 (m, 2H), 6.82 (d, J = 2.0 Hz, 1H), 6.68 (dd, J = 2.0, 8.4 Hz, 1H), 5.11-5.04 (dd, J = 5.5, 12.8 Hz, 1H), 4.94-4.86 (m, 1H), 4.41 (s, 2H), 4.09-3.96 (m, |
| Example 90 | | 1H), 3.81 (q, J = 8.4 Hz, 4H), |
| | | 2.96-2.75 (m, 3H), 2.69-2.55 |
| | | (m, 1H), 2.38-2.22 (m, 3H), |
| | | 2.09-1.95 (m, 1H), 1.86-1.70 |
| | | (m, 4H), 1.44 (d, J = 6.0 Hz, |
| | | 6H), 1.35 (s, 3H). |
| 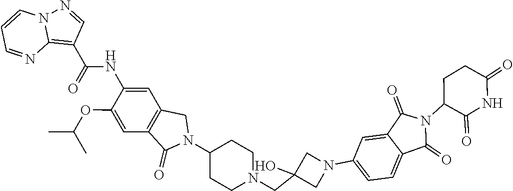 [SMILES: CC(C)Oc1cc2c(cc1NC(=O)c1cnn3cccnc13)CN(C1CCN(CC3(O)CN(c4ccc5c(c4)C(=O)N(C4CCC(=O)NC4=O)C5=O)C3)CC1)C2=O] | 776.2 | DMSO-d₆ δ = 11.08 (s, 1H), 10.74 (s, 1H), 9.40 (dd, J = 1.6, 7.2 Hz, 1H), 8.88 (dd, J = 1.6, 4.4 Hz, 1H), 8.81-8.66 (m, 2H), 8.17 (s, 1H), 7.65 (d, J = 8.4 Hz, 1H), 7.44-7.26 (m, 2H), 6.83 (d, J = 2.0 Hz, 1H), 6.70 (dd, J = 2.0, 8.4 Hz, 1H), 5.08 (dd, J = 5.2, 12.8 Hz, 1H), 4.95-4.82 (m, 1H), 4.37 (s, 2H), 4.11-3.93 (m, 3H), 3.84 (d, J = 8.8 Hz, 2H), 3.14-3.06 |
| Example 61 | | (m, 2H), 2.94-2.83 (m, 1H), |
| | | 2.70-2.51 (m, 5H), 2.33-2.23 |
| | | (m, 2H), 2.06-1.97 (m, 1H), |
| | | 1.87-1.72 (m, 2H), 1.69-1.57 |
| | | (m, 2H), 1.43 (d, J = 6.0 Hz, 6H) |
| 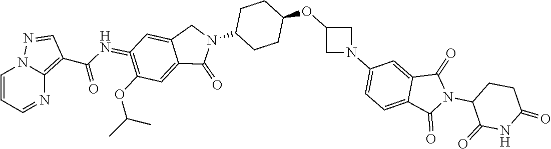 | 761.2 | DMSO-d₆ δ = 11.07 (s, 1 H), 10.75 (s, 1H), 9.39 (dd, J = 1.6, 6.8 Hz, 1 H), 8.88 (dd, J = 1.6, 4.0 Hz, 1 H), 8.73 (d, J = 3.2 Hz, 2 H), 7.64 (d, J = 8.4 Hz, 1 H), 7.43-7.25 (m, 2 H), 6.79 (d, J = 1.6 Hz, 1H), 6.65 (dd, J = 2.4, 8.4 Hz, 1 H), 5.06 (dd, J = 5.2, 12.4 Hz, 1 H), 4.90 (m, 1 H), 4.70-4.55 (m, 1H), 4.39 (s, |
| Example 62 | | 2 H), 4.33-4.23 (m, 2 H), 4.09- |
| | | 3.94 (m, 1 H), 3.84 (m, 2 H), |
| | | 3.47-3.35 (m, 1H), 2.97-2.80 |
| | | (m, 1 H), 2.64-2.52 (m, 2 H), |
| | | 2.12-1.94 (m, 3 H), 1.83-1.73 |
| | | (m, 2 H), 1.72-1.57 (m, 2 H), |
| | | 1.44 (d, J = 6.0 Hz, 6 H), 1.42 - |
| | | 1.33 (m, 2 H) |
| 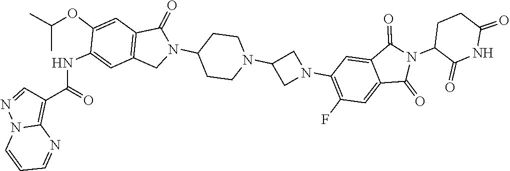 [SMILES: CC(C)Oc1cc2c(cc1NC(=O)c1cnn3cccnc13)CN(C1CCN(C3CN(c4cc5c(cc4F)C(=O)N(C4CCC(=O)NC4=O)C5=O)C3)CC1)C2=O] | 764.4 | DMSO-d₆ δ = 11.09 (s, 1 H), 10.75 (s, 1H), 9.40 (dd, J = 6.8, 1.6 Hz, 1 H), 8.89 (dd, J = 4.4, 1.6 Hz, 1H), 8.73 (s, 2H), 7.61 (d, J = 11.2 Hz, 1H), 7.28-7.39 (m, 2 H), 6.93 (d, J = 7.6 Hz, 1 H), 5.07 (dd, J = 12.8, 5.2 Hz, 1 H), 4.84-4.95 (m, 1 H), 4.42 (s, 2 H), 4.26 (t, J = 6.8 Hz, 2 H), 3.93-4.10 (m, 3 H), 2.82-2.99 (m, 3 H), 2.51-2.63 (m, 3 H), |
| Example 63 | | 1.95-2.07 (m, 3 H), 1.70-1.88 (m, |
| | | 4 H), 1.44 (d, J = 6.0 Hz, 6 H). |
| 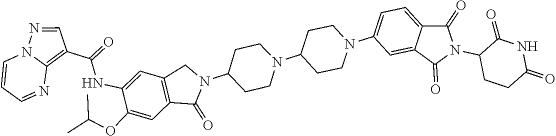 [SMILES: CC(C)Oc1cc2c(cc1NC(=O)c1cnn3cccnc13)CN(C1CCN(C3CCN(c4ccc5c(c4)C(=O)N(C4CCC(=O)NC4=O)C5=O)CC3)CC1)C2=O] | 774.4 | CDCl3 δ = 10.71 (s, 1H), 8.87- 8.82 (m, 2H), 8.79 (s, 1H), 8.73 (dd, J = 1.6, 4.4 Hz, 1H), 8.00- 7.96 (m, 1H), 7.70 (d, J = 8.4 Hz, 1H), 7.38 (s, 1H), 7.31 (d, J = 2.0 Hz, 1H), 7.11-7.05 (m, 2H), 4.99-4.92 (m, 1H), 4.84- 4.76 (m, 1H), 4.34 (s, 2H), 4.32- 4.23 (m, 1H), 4.08-3.99 (m, 2H), |
| Example 64 | | 3.11-2.97 (m, 4H), 2.94-2.72 |
| | | (m, 3H), 2.66-2.56 (m, 1H), 2.46- |
| | | 2.37 (m, 2H), 2.18-2.11 (m, 1H), |
| | | 2.02-1.81 (m, 6H), 1.73-1.63 (m, |
| | | 2H), 1.52 (d, J = 6.0 Hz, 6H) |
| 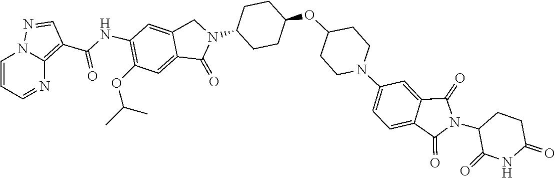 [SMILES: CC(C)Oc1cc2c(cc1NC(=O)c1cnn3cccnc13)CN([C@H]1CC[C@H](OC3CCN(c4ccc5c(c4)C(=O)N(C4CCC(=O)NC4=O)C5=O)CC3)CC1)C2=O] | 789.2 | DMSO-d₆ δ = 11.08 (s, 1H), 10.73 (s, 1 H) 9.39 (dd, J = 7.2, 1.6 Hz, 1 H) 8.88 (dd, J = 4.0, 1.6 Hz, 1 H) 8.73 (s, 2 H) 7.65 (d, J = 8.4 Hz, 1 H) 7.33-7.37 (m, 1 H) 7.32 (s, 2 H) 7.24 (dd, J = 8.8, 2.0 Hz, 1 H) 5.07 (dd, J = 12.8, 5.2 Hz, 1 H) 4.89 (dt, J = 12.0, 6.0 Hz, 1 H) 4.38 (s, 2 H) 3.99 (m, 1 H) 3.80 (m, 2 H) 3.68-3.75 (m, 1 H) 3.44 (m, 1 H) |
| Example 65 | | 3.24 (m, 2 H) 2.81-2.95 (m, 1 H) |
| | | 2.51-2.63 (m, 2 H) 1.97-2.07 (m, |
| | | 3 H) 1.88 (m,2 H) 1.72-1.80 (m, |
| | | 2 H) 1.60-1.70 (m, 2 H) 1.46-1.54 |
| | | (m, 2 H) 1.43 (d, J = 6.0 Hz, 6 H) |
| | | 1.28-1.38 (m, 2 H) 1.23 (m, 1 H) |
| 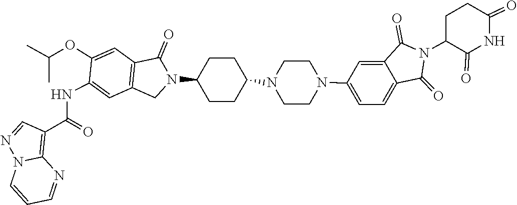 [SMILES: CC(C)Oc1cc2c(cc1NC(=O)c1cnn3cccnc13)CN([C@H]1CC[C@H](N3CCN(c4ccc5c(c4)C(=O)N(C4CCC(=O)NC4=O)C5=O)CC3)CC1)C2=O] | 774.3 | DMSO-d₆ δ = 11.08 (s, 1H), 10.75 (s, 1H), 9.40 (dd, J = 1.6, 6.8 Hz, 1H), 8.89 (dd, J = 1.2, 4.0 Hz, 1H), 8.73 (s, 2H), 7.67 (d, J = 8.0 Hz, 1H), 7.38-7.31 (m, 3H), 7.25 (br d, J = 8.8 Hz, 1H), 5.07 (dd, J = 5.6, 13.2 Hz, 1H), 4.94-4.85 (m, 1H), 4.39 (s, 2H), 4.03-3.94 (m, 1H), 3.47- 3.40 (m, 4H), 2.94-2.83 (m, 1H), 2.70-2.64 (m, 4H), 2.61-2.55 (m, 1H), 2.41-2.34 (m, 1H), 2.04-1.98 (m, 1H), 1.96-1.88 (m, 2H), 1.85- |
| Example 66 | | 1.77 (mz, 2H), 1.70-1.58 (m, 2H), |
| | | 1.48-1.38 (m, 8H), 1.29-1.23 |
| | | (m, 1H) |
| 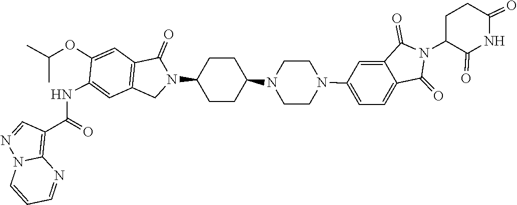 [SMILES: CC(C)Oc1cc2c(cc1NC(=O)c1cnn3cccnc13)CN([C@H]1CC[C@@H](N3CCN(c4ccc5c(c4)C(=O)N(C4CCC(=O)NC4=O)C5=O)CC3)CC1)C2=O] | 774.1 | Methanol-d δ = 9.07-9.17 (m, 1 H) 8.84 (dd, J = 4.0, 1.6 Hz, 1 H) 8.75 (s, 1 H) 8.66 (s, 1 H) 8.10-8.43 (m, 1 H) 7.69 (d, J = 8.8 Hz, 1 H) 7.39 (s, 2 H) 7.26 (dd, J = 6.8, 4.0 Hz, 2 H) 4.51 (s, 2 H) 4.23-4.31 (m, 1 H) 3.55 (br s, 4 H) 2.79-2.85 (m, 4 H) 2.65-2.76 (m, 2 H) 2.39-2.51 (m, 1 H) 1.99-2.29 (m, 6 H) 1.61-1.73 (m, 4 H) 1.51 (d, J = 6.0 Hz, 6H) |
| Example 67 | | |
| 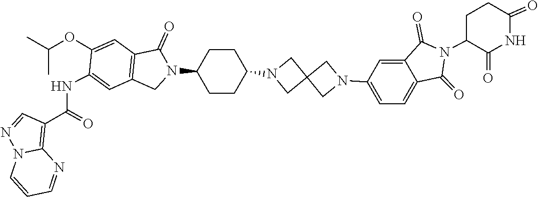 [SMILES: CC(C)Oc1cc2c(cc1NC(=O)c1cnn3cccnc13)CN([C@H]1CC[C@H](N3CC4(CN(c5ccc6c(c5)C(=O)N(C5CCC(=O)NC5=O)C6=O)C4)C3)CC1)C2=O] | 786.4 | DMSO-d₆ δ = 11.09 (s, 1H), 10.76 (s, 1H), 9.40 (dd, J = 1.5, 7.0 Hz, 1H), 8.89 (dd, J = 1.5, 4.3 Hz, 1H), 8.74 (s, 2H), 8.19 (s, 1H), 7.65 (d, J = 8.3 Hz, 1H), 7.43-7.31 (m, 2H), 6.79 (d, J = 1.5 Hz, 1H), 6.64 (dd, J = 1.9, 8.4 Hz, 1H), 5.07 (dd, J = 5.3, 13.1 Hz, 1H), 4.91 (td, J = 5.9, 12.0 Hz, 1H), 4.39 (s, 2H), 4.10 (s, 4H), 4.01-3.93 (m, 1H), 3.33 (br s, 4H), 2.92- |
| Example 68 | | 2.83 (m, 1H), 2.62-2.55 (m, |
| | | 2H), 2.06-1.95 (m, 2H), 1.85- |
| | | 1.69 (m, 4H), 1.64-1.51 (m, |
| | | 2H), 1.44 (d, J = 5.8 Hz, 6H), |
| | | 1.15-1.01 (m, 2H) |
| 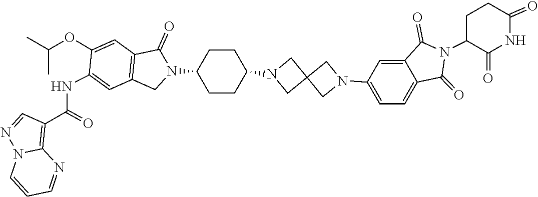 [SMILES: CC(C)Oc1cc2c(cc1NC(=O)c1cnn3cccnc13)CN([C@H]1CC[C@@H](N3CC4(CN(c5ccc6c(c5)C(=O)N(C5CCC(=O)NC5=O)C6=O)C4)C3)CC1)C2=O] | 786.4 | CDCl3: δ ppm 10.71 (s, 1 H) 8.83-8.87 (m, 2 H) 8.80 (s, 1 H) 8.71-8.75 (m, 1 H) 8.06- 8.26 (m, 1 H) 7.67 (d, J = 8.0 Hz, 1 H) 7.39 (s, 1 H) 7.09 (dd, J = 7.2, 4.0 Hz, 1 H) 6.81 (d, J = 1.6 Hz, 1 H) 6.54 (dd, J = 8.4, 1.6 Hz, 1 H) 4.95 (dd, J =12.4, 5.2 Hz, 1 H) 4.76-4.85 (m, 1 H) 4.39 (s, 2 H) 4.18-4.30 (m, 1H) 4.14 (s, 4 H) 3.35 (s, 4 H) 2.69-2.97 (m, 3 H) 2.62 (s, 1 |
| Example 69 | | H) 2.28-2.33 (m, 1 H) 2.11-2.18 |
| | | (m, 1 H) 1.85-1.97 (m, 2 H) |
| | | 1.75 (br d, J = 12.0 Hz, 2 H) |
| | | 1.56 (br d, J = 12.4 Hz, 3 H) 1.52 |
| | | (d, J = 5.6 Hz, 6 H) |
| 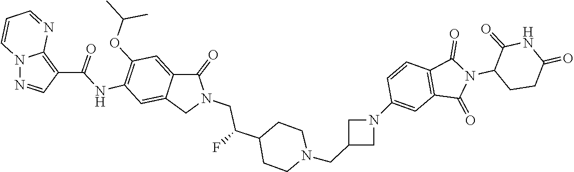 [SMILES: CC(C)Oc1cc2c(cc1NC(=O)c1cnn3cccnc13)CN(C[C@@H](F)C1CCN(CC3CN(c4ccc5c(c4)C(=O)N(C4CCC(=O)NC4=O)C5=O)C3)CC1)C2=O] | 806.3 | DMSO-d₆ δ = 11.07 (s, 1 H) 10.76 (s, 1 H) 9.40 (dd, J = 6.8, 1.6 Hz, 1H) 8.89 (dd, J = 4.4, 1.6 Hz, 1 H) 8.74 (s, 2 H) 7.62 (d, J = 8.4 Hz, 1 H) 7.32-7.39 (m, 2 H) 6.75 (d, J =1.6 Hz, 1 H) 6.62 (dd, J = 8.4, 2.4 Hz, 1 H) 5.05 (dd, J = 12.4, 5.2 Hz, 1 H) 4.91 (dt, J = 12.0, 6.0 Hz, 1 H) 4.52-4.68 (m, 1H) 4.42-4.51 (m, 2 H) 4.11 (br t, J = 8.0 Hz, 2 |
| Example 70 | | H) 3.69-3.90 (m, 2 H) 3.60- |
| | | 3.69 (m, 2 H) 2.93-3.05 (m, 1 |
| | | H) 2.79-2.92 (m, 3 H) 2.54- |
| | | 2.61 (m, 3 H) 1.97-2.06 (m, 1 |
| | | H) 1.84-1.96 (m, 2 H) 1.66-1.82 |
| | | (m, 2 H) 1.44 (d, J = 6.0 Hz, |
| | | 6H) 1.19-1.39 (m, 4H) |
| 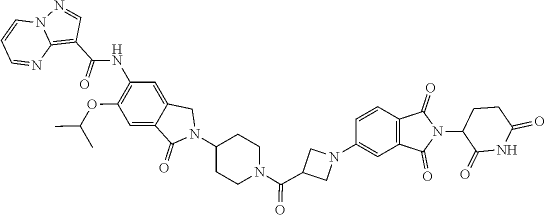 [SMILES: CC(C)Oc1cc2c(cc1NC(=O)c1cnn3cccnc13)CN(C1CCN(C(=O)C3CN(c4ccc5c(c4)C(=O)N(C4CCC(=O)NC4=O)C5=O)C3)CC1)C2=O] | 774.4 | DMSO-d₆ δ = 11.08 (s, 1H), 10.77 (s, 1H), 9.41 (dd, J = 1.6, 7.2 Hz, 1H), 8.90 (dd, J = 1.6, 4.4 Hz, 1H), 8.74 (s, 2H), 7.67 (d, J = 8.0 Hz, 1H), 7.43-7.29 (m, 2H), 6.85 (d, J = 2.0 Hz, 1H), 6.71 (dd, J = 2.0, 8.4 Hz, 1H), 5.07 (dd, J = 5.2, 12.8 Hz, 1H), 4.91 (td, J = 6.0, 12.0 Hz, 1H), 4.67-4.37 (m, 4H), 4.36-4.14 (m, 5H), 3.97 (br d, J = 7.2 Hz, 1H),3.74 (br d, J = 12.4 Hz, 1H), 3.20 (br s, 1H), 2.95-2.74 (m, 2H), 2.02 (br d, J = 5.2 Hz, 1H), |
| Example 71 | | 1.77 (br d, J = 9.2 Hz, 5H), 1.44 |
| | | (d, J = 6.0 Hz, 6H) |
| 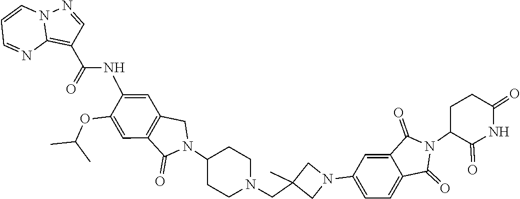 [SMILES: CC(C)Oc1cc2c(cc1NC(=O)c1cnn3cccnc13)CN(C1CCN(CC3(C)CN(c4ccc5c(c4)C(=O)N(C4CCC(=O)NC4=O)C5=O)C3)CC1)C2=O] | 774.3 | DMSO-d₆ δ = 11.07 (s, 1 H), 10.59 (s, 1 H), 9.38 (dd, J = 1.6, 7.0 Hz, 1 H), 8.87 (dd, J = 1.2, 4.0 Hz, 1 H), 8.72 (d, J = 13.6 Hz, 2 H), 8.30 (s, 1 H), 7.64 (d, J = 8.4 Hz, 1 H), 7.34 (dd, J = 4.4, 6.8 Hz, 1 H), 7.13 (s, 1 H), 6.79 (d, J = 2.0 Hz, 1 H), 6.66 (dd, J = 2.0, 8.4 Hz, 1 H), 5.06 (dd,J = 5.2, 12.8 Hz, 1 H), 4.91- 4.79 (m, 1 H), 4.43-4.29 (m, 1H), 3.83-3.75 (m, 4H), 3.29 (s, 1 H), 2.87 (m, 3 H), 2.58 (s, |
| Example 72 | | 2H), 2.35-2.22 (m, 3H), 2.10- |
| | | 2.04 (m, 3 H), 1.48 (s, 3 H), |
| | | 1.46 (s, 3H), 1.39 (s, 3 H), 0.94 |
| | | (d, J = 6.8 Hz, 2 H) |
| 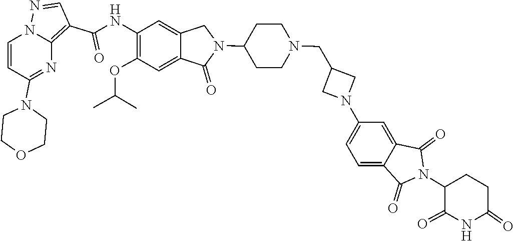 [SMILES: CC(C)Oc1cc2c(cc1NC(=O)c1cnn3ccc(N4CCOCC4)nc13)CN(C1CCN(CC3CN(c4ccc5c(c4)C(=O)N(C4CCC(=O)NC4=O)C5=O)C3)CC1)C2=O] | 845.2 | DMSO-d₆ δ = 11.08 (s, 1H), 9.70 (s, 1H), 8.86 (d, J = 8.0 Hz, 1H), 8.41-8.28 (m, 2H), 7.63 (d, J = 8.0 Hz, 1H), 7.32 (s, 1H), 6.95 (d, J = 8.0 Hz, 1H), 6.78 (d, J = 2.0 Hz, 1H), 6.65 (dd, J = 2.0, 8.4 Hz, 1H), 5.05 (dd, J = 5.2, 12.8 Hz, 1H), 4.71-4.61 (m, 1H), 4.44-4.36 (m, 2H), 4.14 (t, J = 8.0 Hz, 2H), 4.03-3.93 (m, 1H), 3.88- 3.66 (m, 10H), 3.06-2.83 (m, 4H), 2.65-2.56 (m, 4H), 2.15- 1.95 (m, 3H), 1.89-1.61 (m, 4H), 1.27 (d, J = 6.0 Hz, 6H) |
| Example 73 | | |
| 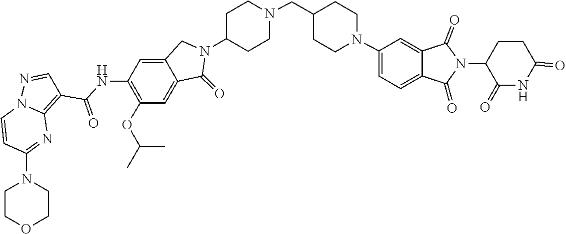 [SMILES: CC(C)Oc1cc2c(cc1NC(=O)c1cnn3ccc(N4CCOCC4)nc13)CN(C1CCN(CC3CCN(c4ccc5c(c4)C(=O)N(C4CCC(=O)NC4=O)C5=O)CC3)CC1)C2=O] | 837.2 | DMSO-d₆ δ = 11.08 (s, 1H), 9.70 (s, 1H), 8.86 (d, J = 8.0 Hz, 1H), 8.38-8.25 (m, 2H), 7.65 (d, J = 8.8 Hz, 1H), 7.31 (s, 2H), 7.25-7.18 (m, 1H), 6.94 (d, J = 8.0 Hz, 1H), 5.06 (dd, J = 5.2, 12.8 Hz, 1H), 4.72- 4.60 (m, 1H), 4.40 (s, 2H), 4.10-3.94 (m, 3H), 3.84-3.69 (m, 8H), 3.04-2.84 (m, 5H), 2.62-2.54 (m, 2H), 2.22-2.11 (m, 2H), 2.06-1.95 (m, 3H), 1.86-1.77 (m, 4H), 1.70-1.66 (m, 1H), 1.32-1.21 (m, 7H), 1.20- 1.09 (m, 2H), 0.88-0.79 (m, 1H) |
| Example 74 | | |
| 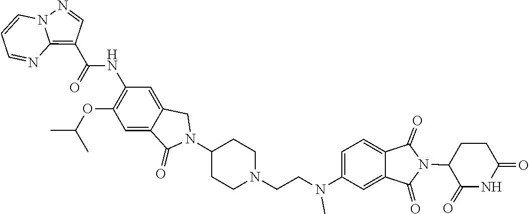 [SMILES: CC(C)Oc1cc2c(cc1NC(=O)c1cnn3cccnc13)CN(C1CCN(CCN(C)c3ccc4c(c3)C(=O)N(C3CCC(=O)NC3=O)C4=O)CC1)C2=O] | 748.3 | DMSO-d₆ δ = 11.05 (s, 1 H), 10.75 (s, 1H), 9.40(dd, J = 1.6, 6.8 Hz, 1 H), 8.89 (dd, J = 1.6, 4.4 Hz, 1 H), 8.73 (d, J = 2.0 Hz, 2 H), 7.65 (d, J = 8.4 Hz, 1 H), 7.42-7.28 (m, 2 H), 7.12 (d, J = 2.4 Hz, 1H), 7.00 (dd, J = 2.0, 8.4 Hz, 1 H), 5.07 (dd, J = 5.6, 12.8 Hz, 1 H), 4.89 (m, 1 H), 4.37 (s, 2 H), 4.08-3.90 (m, 1 H), 3.65 (m, 2 H), 3.10 (s, 3 H), 3.01 (m, 2 H), 2.89 (m, 1 H), 2.63-2.52 (m, 4 H), 2.13 (m, 2 H), 2.06-1.96 (m, 1 H), |
| Example 75 | | 1.83-1.61 (m, 4 H), 1.43 (d, J = |
| | | 6.0 Hz, 6 H) |
| 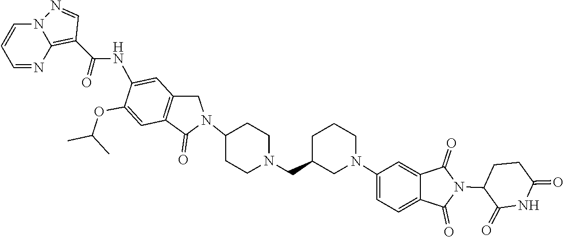 [SMILES: CC(C)Oc1cc2c(cc1NC(=O)c1cnn3cccnc13)CN(C1CCN(C[C@H]3CCCN(c4ccc5c(c4)C(=O)N(C4CCC(=O)NC4=O)C5=O)C3)CC1)C2=O] | 788.3 | DMSO-d₆ δ = 11.05 (s, 1 H) 10.76 (s, 1 H) 9.40 (d, J = 7.2 Hz, 1 H) 8.89 (d, J = 3.2 Hz, 1 H) 8.75 (d, J = 7.2 Hz, 2 H) 7.67 (d, J = 8.4 Hz, 1 H) 7.33-7.38 (m, 2 H) 7.31 (s, 1 H) 7.22 (br d, J = 9.2 Hz, 1 H) 5.06 (dd, J = 12.8, 5.2 Hz, 1H) 4.90 (m, 1H) 4.46 (s, 2 H) 4.01 (br s, 1 H) 3.78- 3.91 (m, 2 H) 3.18 (br s, 1 H) 3.00 (br s, 2 H) 2.80-2.94 (m, 2 H) 2.54-2.69 (m, 2 H) 2.33 (br s, 1 H) 1.94-2.16 (m, 5 H) 1.86 (m, 4 H) 1.70 (br s, 3 H) 1.54 (br s, 1 H) 1.44 (d, J = 5.6 |
| Example 76 | | Hz, 6 H). |
| 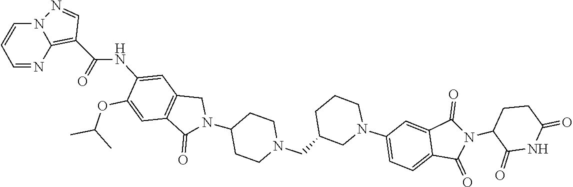 [SMILES: CC(C)Oc1cc2c(cc1NC(=O)c1cnn3cccnc13)CN(C1CCN(C[C@@H]3CCCN(c4ccc5c(c4)C(=O)N(C4CCC(=O)NC4=O)C5=O)C3)CC1)C2=O] | 788.3 | DMSO-d₆ δ = 11.07 (s, 1H), 10.77 (s, 1H), 9.41 (dd, J = 1.6, 7.2 Hz, 1H), 8.90 (dd, J = 1.6, 4.4 Hz, 1H), 8.81-8.71 (m, 2H), 7.69 (d, J = 8.8 Hz, 1H), 7.41-7.33 (m, 3H), 7.30-7.23 (m, 1H), 5.07 (dd, J = 5.6, 12.8 Hz, 1H), 4.98-4.84 (m, 1H), 4.47 (s, 2H), 4.22-4.05 (m, 1H), 4.05-3.83 (m, 2H), 3.43-3.36 (m, 3H), 3.18-3.03 (m, 2H), 2.96-2.83 (m, 2H), 2.64-2.53 (m, 4H), 2.09- |
| Example 77 | | 1.73 (m, 8H), 1.62-1.52 (m, 1H), |
| | | 1.45 (d, J = 6.0 Hz, 6H), 1.36-1.20 |
| | | (m, 1H) |
| 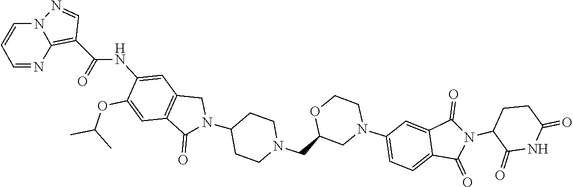 [SMILES: CC(C)Oc1cc2c(cc1NC(=O)c1cnn3cccnc13)CN(C1CCN(C[C@@H]3CN(c4ccc5c(c4)C(=O)N(C4CCC(=O)NC4=O)C5=O)CCO3)CC1)C2=O] | 790.2 | DMSO-d₆ δ = 11.09 (s, 1 H), 10.77 (s, 1H),9.41 (dd, J = 1.6, 6.8 Hz, 1 H), 8.90 (dd, J = 1.6, 4.0 Hz, 1 H), 8.80-8.69 (m, 2 H), 7.75 (d, J = 8.4 Hz, 1 H), 7.41 (s, 1 H), 7.39-7.34 (m, 2 H), 7.30 (dd, J = 2.0, 8.8 Hz, 1 H), 5.09 (dd, J = 5.2, 12.8 Hz, 1 H), 4.91 (m, 1 H), 4.44 (m, 2 H),4.27 (s, 1H), 4.09-3.88 (m, 4 H), 3.82-3.62 (m, 2H), 3.36 (m, 2 H), 3.06-2.91 (m, 2 H), |
| Example 78 | | 2.91-2.84 (m, 1H), 2.83-2.68 |
| | | (m, 2 H), 2.65-2.53 (m, 4 H), |
| | | 2.10-1.97 (m,2H), 1.95-1.79 (m, |
| | | 2H), 1.44 (d, J = 6.0 Hz, 6 H) |
| 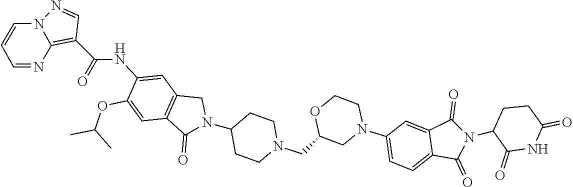 [SMILES: CC(C)Oc1cc2c(cc1NC(=O)c1cnn3cccnc13)CN(C1CCN(C[C@H]3CN(c4ccc5c(c4)C(=O)N(C4CCC(=O)NC4=O)C5=O)CCO3)CC1)C2=O] | 790.3 | DMSO-d₆ δ = 11.09 (s, 1H), 10.77 (s, 1H), 9.43-9.39 (m, 1H), 8.91-8.88 (m, 1H), 8.77 (s, 1H), 8.74 (s, 1H), 7.75 (br d, J = 8.4 Hz, 1H), 7.43-7.34 (m, 3H), 7.30 (d, J = 8.4 Hz, 1H), 5.12-5.06 (m, 1H), 4.94-4.86 (m, 1H), 4.44 (br s, 2H), 4.07- 3.86 (m, 4H), 3.70 (br s, 2H), 3.25-3.09 (m, 2H), 3.06-2.69 (m, 5H), 2.62 (br s, 1H), 2.59- 2.53 (m, 2H), 2.29-1.63 (m, |
| Example 79 | | 6H), 1.44 (d, J = 6.0 Hz, 6H) |
| 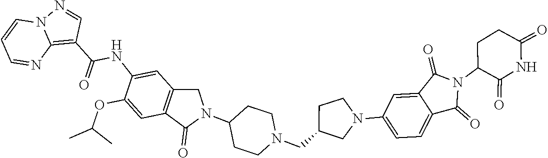 [SMILES: CC(C)Oc1cc2c(cc1NC(=O)c1cnn3cccnc13)CN(C1CCN(C[C@@H]3CCN(c4ccc5c(c4)C(=O)N(C4CCC(=O)NC4=O)C5=O)C3)CC1)C2=O] | 774.2 | DMSO-d₆ δ = 11.07 (s, 1H), 10.74 (s, 1H), 9.38 (dd, J = 1.6, 7.2 Hz, 1 H), 8.88 (dd, J = 1.6, 4.4 Hz, 1 H), 8.78-8.66 (m, 2 H), 8.24 (s, 1 H), 7.63 (d, J = 8.4 Hz, 1 H), 7.42-7.26 (m, 2 H), 6.88 (d, J = 2.0 Hz, 1 H), 6.79 (dd, J = 2.0, 8.8 Hz, 1 H), 5.06 (dd, J = 5.2, 12.8 Hz, 1H), 4.89 (m, 1 H), 4.43 (s, 2 H), |
| Example 80 | | 4.04-3.93 (m, 1H), 3.40-3.31 |
| | | (m, 3 H), 3.13 (m, 1 H), 3.04 |
| | | (m, 1 H), 2.99-2.86 (m, 2 H), |
| | | 2.65-2.51 (m, 4 H), 2.38 (m, 2 |
| | | H), 2.18-1.94 (m, 4 H), 1.92- |
| | | 1.63 (m, 5 H), 1.44 (d, J = 6.0 |
| | | Hz, 6 H) |
| 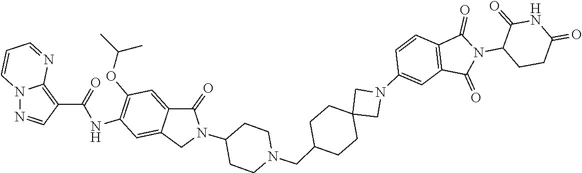 [SMILES: CC(C)Oc1cc2c(cc1NC(=O)c1cnn3cccnc13)CN(C1CCN(CC3CCC4(CC3)CN(c3ccc5c(c3)C(=O)N(C3CCC(=O)NC3=O)C5=O)C4)CC1)C2=O] | 828.3 | DMSO-d₆ δ = 11.09 (s, 1H), 10.78 (s, 1H), 9.42 (dd, J = 1.6, 7.2 Hz, 1H), 8.91 (dd, J = 1.6, 4.4 Hz, 1H), 8.82-8.68 (m, 2H), 7.65 (d, J = 8.4 Hz, 1H), 7.41- 7.31 (m, 2H), 6.78 (d, J = 1.6 Hz, 1H), 6.65 (dd, J = 2.0, 8.4 Hz, 1H), 5.12-5.01 (m, 1H), 4.91 (td, J = 6.0, 12.0 Hz, 1H), 4.44 (s, 2H), 4.35-3.93 (m, 1H), 3.80-3.66 (m, 4H), 3.31-3.15 |
| Example 81 | | (m, 2H), 3.04-2.77 (m, 2H), |
| | | 2.62-2.53 (m, 2H), 2.49-2.39 |
| | | (m, 2H), 2.13-1.87 (m, 6H), 1.76 |
| | | (br dd, J = 7.2, 11.2 Hz, 5H), |
| | | 1.58-1.50 (m, 2H), 1.45 (d, J = |
| | | 6.0 Hz, 6H), 1.09-0.93 (m, 2H). |
| 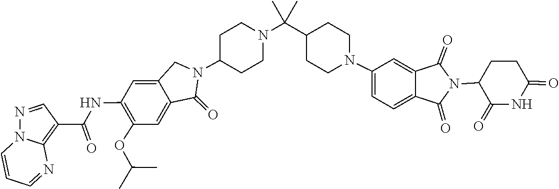 [SMILES: CC(C)Oc1cc2c(cc1NC(=O)c1cnn3cccnc13)CN(C1CCN(C(C)(C)C3CCN(c4ccc5c(c4)C(=O)N(C4CCC(=O)NC4=O)C5=O)CC3)CC1)C2=O] | 816.5 | CDCl3 δ = 10.72 (s, 1H), 8.85 (s, 2H), 8.80 (s, 1H), 8.75-8.70 (m, 1H), 7.94 (s, 1H), 7.71- 7.66 (m, 1H), 7.39 (s, 1H), 7.32- 7.29 (m, 1H), 7.11-7.05 (m, 2H), 4.99-4.92 (m, 1H), 4.85- 4.77 (m, 1H), 4.35 (s, 2H), 4.31- 4.21 (m, 1H), 4.08-4.02 (m, 2H), 3.15-3.07 (m, 2H), 2.98- 2.87 (m, 3H), 2.83-2.71 (m, 2H), 2.36-2.26 (m, 2H), 2.18- 2.10 (m, 1H), 1.95-1.84 (m, |
| Example 82 | | 1H), 1.95-1.84 (m, 4H), 1.77- |
| | | 1.72 (m, 2H), 1.52 (d, J = 6.0 |
| | | Hz, 7H), 1.49-1.39 (m, 2H), |
| | | 0.99-0.96 (m, 6H) |
| 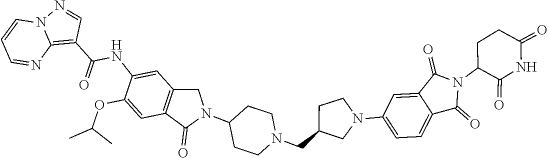 [SMILES: CC(C)Oc1cc2c(cc1NC(=O)c1cnn3cccnc13)CN(C1CCN(C[C@H]3CCN(c4ccc5c(c4)C(=O)N(C4CCC(=O)NC4=O)C5=O)C3)CC1)C2=O] | 774.3 | DMSO-d₆ δ = 11.07 (s, 1H), 10.75 (s, 1H), 9.39 (d, J = 6.8 Hz, 1H), 8.89 (d, J = 4.0 Hz, 1H), 8.74 (d, J = 3.6 Hz, 2H), 8.19 (s, 1H), 7.64 (d, J = 8.4 Hz, 1H), 7.38-7.31 (m, 2H), 6.89 (s, 1H), 6.81 (d, J = 8.0 Hz, 1H), 5.06 (dd, J = 5.2, 12.8 Hz, 1H), 4.94-4.85 (m, 1H), 4.43 (s, 2H), 4.00 (s, 1H), 3.18- |
| Example 83 | | 2.82 (m, 7H), 2.65-2.53 (m, |
| | | 3H), 2.39 (d, J = 7.2 Hz, 2H), |
| | | 2.17-1.98 (m, 4H), 1.87-1.67 |
| | | (m, 5H), 1.44 (d, J = 6.0 Hz, 6H) |
| 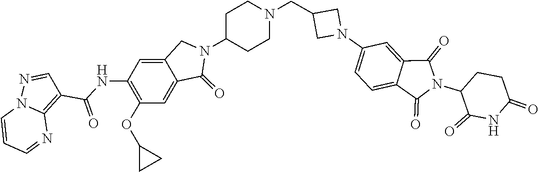 [SMILES: O=C1CCC(N2C(=O)c3ccc(N4CC(CN5CCC(N6Cc7cc(NC(=O)c8cnn9cccnc89)c(OC8CC8)cc7C6=O)CC5)C4)cc3C2=O)C(=O)N1] | 758.2 | DMSO-d₆ δ = 11.07 (s, 1 H), 10.57 (s, 1H), 9.38 (dd, J = 1.6, 6.8 Hz, 1 H), 8.90 (dd, J = 1.6, 4.0 Hz, 1 H), 8.71 (d, J = 7.6 Hz, 2 H), 7.72-7.48 (m, 2 H), 7.41-7.25 (m, 1 H), 6.76 (d, J = 2.0 Hz, 1 H), 6.63 (dd, J = 2.0, 8.4 Hz, 1 H), 5.05 (dd, J = 5.2, 12.8 Hz, 1 H), 4.43 (s, 2 H), 4.21 (m, 1 H), 4.13 (m, 2 H), 4.06-3.93 (m, 1 H), 3.69 (m, 2 |
| Example 84 | | H), 3.02-2.90 (m, 3 H), 2.89- |
| | | 2.82 (m, 1 H), 2.66-2.53 (m, 4 |
| | | H), 2.16-1.96 (m, 3 H), 1.87- |
| | | 1.62 (m, 4 H), 0.99-0.90 (m, 2 |
| | | H), 0.89-0.82 (m, 2 H) |
| 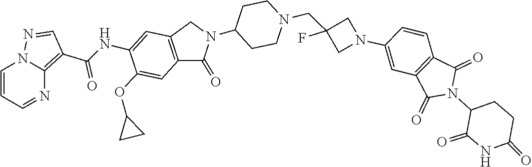 [SMILES: O=C1CCC(N2C(=O)c3ccc(N4CC(F)(CN5CCC(N6Cc7cc(NC(=O)c8cnn9cccnc89)c(OC8CC8)cc7C6=O)CC5)C4)cc3C2=O)C(=O)N1] | 776.2 | DMSO-d₆ δ = 11.08 (s, 1H), 10.58 (s, 1H), 9.40 (dd, J = 1.6, 7.2 Hz, 1H), 8.91 (dd, J = 1.6, 4.0 Hz, 1H), 8.73 (s, 1H), 8.70 (s, 1H), 7.69 (d, J = 8.0 Hz, 1H), 7.57 (s, 1H), 7.36 (dd, J = 4.4, 7.2 Hz, 1H), 6.92 (d, J = 1.6 Hz, 1H), 6.78 (dd, J = 2.0, 8.0 Hz, 1H), 5.08 (dd, J = 5.2, 12.8 Hz, 1H), 4.42 (s, 2H), 4.29- 4.11 (m, 5H), 4.06-3.95 (m, |
| Example 85 | | 1H), 3.03 (br d, J = 11.2 Hz, |
| | | 2H), 2.96-2.83 (m, 3H), 2.63- |
| | | 2.54 (m, 2H), 2.38-2.29 (m, |
| | | 2H), 2.07-1.97 (m, 1H), 1.88- |
| | | 1.75 (m, 2H), 1.73-1.63 (m, |
| | | 2H), 0.98-0.91 (m, 2H), 0.89- |
| | | 0.83 (m, 2H) |
| 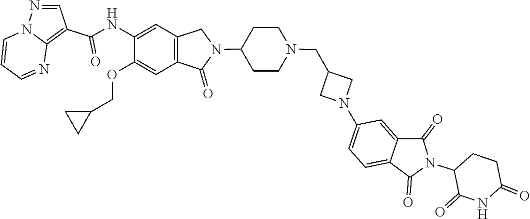 [SMILES: O=C1CCC(N2C(=O)c3ccc(N4CC(CN5CCC(N6Cc7cc(NC(=O)c8cnn9cccnc89)c(OCC8CC8)cc7C6=O)CC5)C4)cc3C2=O)C(=O)N1] | 772.3 | DMSO-d₆ δ = 11.08 (s, 1 H) 10.83 (s, 1 H) 9.39 (d, J = 7.2 Hz, 1 H) 8.88 (d, J = 2, 1 H) 8.74 (s, 2 H) 7.63 (d, J = 8.4 Hz, 1 H) 7.34 (dd, J = 6.8, 4.0 Hz, 1 H) 7.26 (s, 1 H) 6.78 (s, 1 H) 6.65 (d, J = 8.4 Hz, 1 H) 5.05 (dd, J = 12.8, 5.2 Hz, 1 H) 4.43 (s, 2 H) 4.14 (t, J = 8.0 Hz, 2 H) 4.08- 4.07 (m, 2 H) 3.95-4.04 (m, 1 H) 3.67-3.73 (m, 2 H) 2.97- 2.92 (m, 4 H) 2.65-2.55 (m, 3 H) 2.14-1.97 (m, 4 H) 1.84- 1.73 (m, 2 H) 1.72-1.65 (m, 2 |
| Example 86 | | H) 1.50-1.41 (m, 1 H) 0.72-0.66 |
| | | (m, 2 H) 0.47-0.42 (m, 2 H) |
| 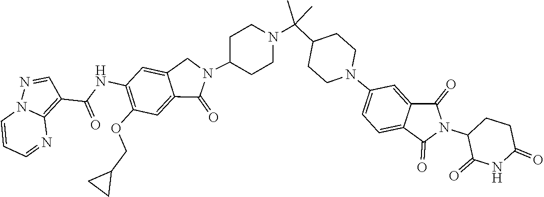 [SMILES: CC(C)(C1CCN(c2ccc3c(c2)C(=O)N(C2CCC(=O)NC2=O)C3=O)CC1)N1CCC(N2Cc3cc(NC(=O)c4cnn5cccnc45)c(OCC4CC4)cc3C2=O)CC1] | 828.5 | CDCl3 δ = 10.71 (s, 1H), 8.89- 8.84 (m, 2H), 8.80 (s, 1H), 8.72 (dd, J = 1.6, 4.0 Hz, 1H), 7.95 (s, 1H), 7.69 (d, J = 8.8 Hz, 1H), 7.34 (s, 1H), 7.30 (d, J = 2.4 Hz, 1H), 7.10-7.03 (m, 2H), 4.95 (dd, J = 5.6, 12.4 Hz, 1H), 4.35 (s, 2H), 4.30-4.19 (m, 1H), 4.09-4.01 (m, 4H), 3.11 (br d, J = 12 Hz, 2H), 2.97- 2.87 (m, 3H), 2.87-2.62 (m, 2H), 2.30 (br t, J = 10 Hz, 2H), 2.19-2.10 (m, 1H), 1.89 (br t, |
| Example 87 | | J = 13.6 Hz, 4H), 1.83-1.77 |
| | | (m, 1H), 1.77-1.70 (m, 2H), |
| | | 1.70-1.58 (m, 4H), 1.53 -1.33 |
| | | (m, 4H), 1.01-0.93 (m, 6H), 0.78- |
| | | 0.69 (m, 2H), 0.56-0.49 (m, 2H) |
| 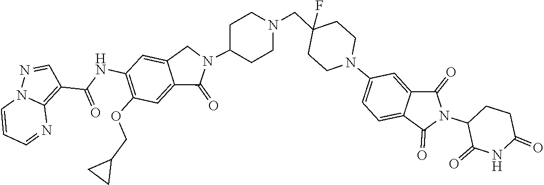 [SMILES: O=C1CCC(N2C(=O)c3ccc(N4CCC(F)(CN5CCC(N6Cc7cc(NC(=O)c8cnn9cccnc89)c(OCC8CC8)cc7C6=O)CC5)CC4)cc3C2=O)C(=O)N1] | 818.3 | CDCl3 δ = 10.70 (s, 1H), 8.83- 8.92 (m, 3 H), 8.71 (dd, J = 4.0, 1.6 Hz, 1 H), 8.58 (s, 1 H), 7.72 (d, J = 8.4 Hz, 1 H), 7.30-7.36 (m, 2 H), 7.02-7.14 (m, 2 H), 4.97 (dd, J = 12.0, 5.2 Hz, 1H), 4.30 (s, 2 H), 4.17-4.26 (m, 1 H), 4.05 (d, J = 6.8 Hz, 2 H), 3.83 (d, J = 13.2 Hz, 2 H), 3.41 (t, J = 12.4 Hz, 2 H), 2.73-3.04 (m, 5 H), 2.54 (d, J = 19.6 Hz, 2 H), 2.41 (s, 2 H), 2.12-2.22 |
| Example 88 | | (m, 1 H), 1.94-2.04 (m, 2 H), |
| | | 1.76-1.91 (m, 6 H), 1.41-1.51 |
| | | (m, 1 H), 0.66-0.76 (m, 2 H), |
| | | 0.52 (q, J = 4.8 Hz, 2 H). |
| 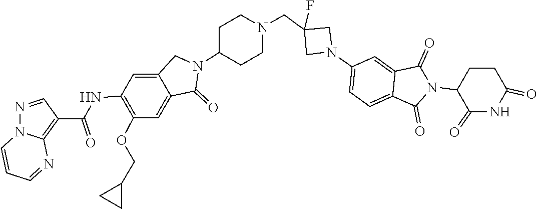 [SMILES: O=C1CCC(N2C(=O)c3ccc(N4CC(F)(CN5CCC(N6Cc7cc(NC(=O)c8cnn9cccnc89)c(OCC8CC8)cc7C6=O)CC5)C4)cc3C2=O)C(=O)N1] | 790.2 | CDCl3 δ = 10.70 (s, 1H), 8.89 (s, 1H), 8.87-8.83 (m, 2H), 8.72 (dd, J = 2.0, 4.4 Hz, 1H), 8.49 (br s, 1H), 7.71 (d, J = 8.0 Hz, 1H), 7.32 (s, 1H), 7.07 (dd, J = 4.0, 6.8 Hz, 1H), 6.89 (d, J = 2.0 Hz, 1H), 6.62 (dd, J = 2.0, 8.4 Hz, 1H), 4.97 (dd, J = 5.2, 12.4 Hz, 1H), 4.28 (s, 2H), 4.26-4.18 (m, 3H), 4.16 (s, 2H), 4.05 (d, J = 6.8 Hz, 2H), 3.04 (br d, J = 10.8 Hz, 2H), 2.94-2.74 (m, 5H), 2.47 (br t, |
| Example 89 | | J = 10.8 Hz, 2H), 2.20-2.12 |
| | | (m, 1H), 1.86-1.73 (m, 4H), |
| | | 1.50-1.44 (m, 1H), 0.76-0.69 |
| | | (m, 2H), 0.55-0.49 (m, 2H) |
| 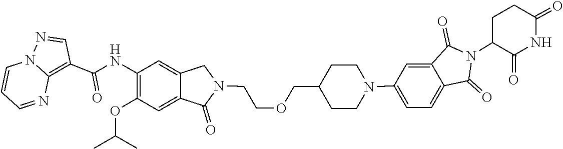 [SMILES: CC(C)Oc1cc2c(cc1NC(=O)c1cnn3cccnc13)CN(CCOCC1CCN(c3ccc4c(c3)C(=O)N(C3CCC(=O)NC3=O)C4=O)CC1)C2=O] | 749.3 | CDCl3 δ = 10.72 (s, 1H), 8.88- 8.83 (m, 2H), 8.79 (s, 1H), 8.73 (dd, J = 2.0, 4.4 Hz, 1H), 8.01- 7.94 (m, 1H), 7.67 (d, J = 8.4 Hz, 1H), 7.39 (s, 1H), 7.09 (dd, J = 4.0, 6.8 Hz, 1H), 7.05 (dd, J = 2.4, 8.4 Hz, 1H), 4.94 (dd, J = 5.2, 12.4 Hz, 1H), 4.80 (td, J = 6.0, 12.0 Hz, 1H), 4.49 (s, 2H), 3.96 (br d, J = 12.4 Hz, |
| Example 90 | | 2H), 3.84-3.78 (m, 2H), 3.73- |
| | | 3.66 (m, 2H), 3.50 (s, 1H), 3.34 |
| | | (d, J = 6.0 Hz, 2H), 3.04-2.65 |
| | | (m, 5H), 2.19-2.09 (m, 1H), |
| | | 1.94-1.79 (m, 3H), 1.52 (d, J = |
| | | 6.0 Hz, 6H), 1.41-1.28 (m, 2H) |
| 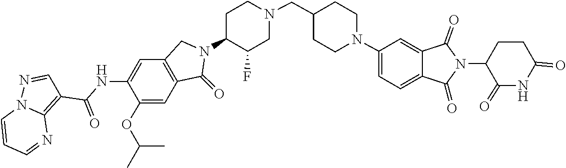 [SMILES: CC(C)Oc1cc2c(cc1NC(=O)c1cnn3cccnc13)CN([C@H]1CCN(CC3CCN(c4ccc5c(c4)C(=O)N(C4CCC(=O)NC4=O)C5=O)CC3)C[C@@H]1F)C2=O] | 806.2 | DMSO-d₆ δ = 11.08 (s, 1H), 10.78 (s, 1H), 9.41 (dd, J = 1.6, 6.8 Hz, 1H), 8.90 (dd, J = 1.6, 4.4 Hz, 1H), 8.76 (s, 1H), 8.74 (s, 1H), 7.66 (d, J = 8.8 Hz, 1H), 7.38-7.34 (m, 2H), 7.32 (d, J = 2.0 Hz, 1H), 7.24 (dd, J = 2.0, 8.8 Hz, 1H), 5.07 (dd, J = 5.2, 12.8 Hz, 1H), 4.95-4.72 (m, 2H), 4.51 (s, 2H), 4.21- 4.10 (m, 1H), 4.06 (br d, J = |
| Example 91 | | 13.2 Hz, 2H), 3.04-2.94 (m, |
| | | 2H), 2.93-2.83 (m, 2H), 2.68- |
| | | 2.54 (m, 2H), 2.35-2.30 (m, |
| | | 1H), 2.27 (br d, J = 5.6 Hz, 2H), |
| | | 2.14-1.97 (m, 3H), 1.92-1.71 |
| | | (m, 5H), 1.44 (d, J = 6.0 Hz, |
| | | 6H), 1.22-1.11 (m, 2H) |
| 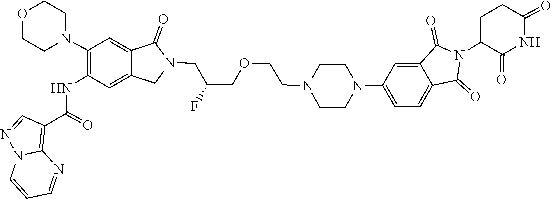 [SMILES: O=C1CCC(N2C(=O)c3ccc(N4CCN(CCOC[C@H](F)CN5Cc6cc(NC(=O)c7cnn8cccnc78)c(N7CCOCC7)cc6C5=O)CC4)cc3C2=O)C(=O)N1] | 823.3 | DMSO-d₆ δ = 11.06 (s, 1 H), 10.88 (s, 1H), 9.40 (dd, J = 1.6, 7.2 Hz, 1H), 8.99 (dd, J = 1.6, 4.4 Hz, 1 H), 8.76 (d, J = 7.2 Hz, 2 H), 8.24-8.14 (m, 1 H), 7.67-7.54 (m, 2 H), 7.39 (dd, J = 4.2, 7.2 Hz, 1H), 7.32 (d, J = 2.0 Hz, 1 H), 7.22 (dd, J = 2.2, 8.8 Hz, 1 H), 5.11-4.81 (m, 2 H), 4.63-4.46 (m, 2 H), 3.95- 3.84 (m, 5H), 3.74-3.55 (m, 6 H), 3.42-3.40 (m, 2 H), 2.95- |
| Example 92 | | 2.83 (m, 6 H), 2.58-2.53 (m, 7 |
| | | H), 2.03-1.97 (m, 1 H), 1.24 |
| | | (br s, 1 H) |
| 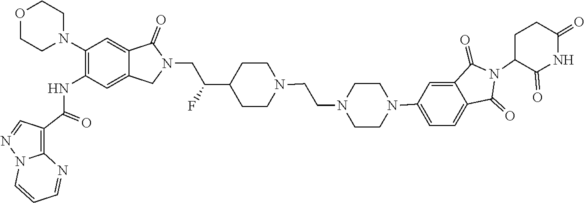 [SMILES: O=C1CCC(N2C(=O)c3ccc(N4CCN(CCN5CCC([C@H](F)CN6Cc7cc(NC(=O)c8cnn9cccnc89)c(N8CCOCC8)cc7C6=O)CC5)CC4)cc3C2=O)C(=O)N1] | 876.2 | DMSO-d₆ δ = 11.07 (s, 1 H), 10.90 (s, 1H), 9.41 (dd, J = 1.6, 7.2 Hz, 1 H), 9.00 (dd, J = 1.6, 4.2 Hz, 1 H), 8.76 (s, 2 H), 7.67 (d, J = 8.8 Hz, 1 H), 7.60 (s, 1 H), 7.39 (dd, J = 4.4, 7.0 Hz, 1 H),7.33 (s, 1 H), 7.25 (br d, J = 8.8 Hz, 1 H), 5.07 (dd, J = 5.2, 12.8 Hz, 1 H), 4.68-4.46 (m, 3 H), 3.94-3.87 (m, 4 H), 3.83- 3.67 (m, 2 H), 3.41 (br d, J = 4.8 Hz, 10 H), 3.00-2.87 (m, 7 H), 2.63-2.54 (m, 4 H), 2.04-1.86 |
| Example 93 | | (m, 3 H), 1.83-1.69 (m, 2 H), 1.56 |
| | | (br s, 1 H), 1.41-1.25 (m, 2 H) |
| 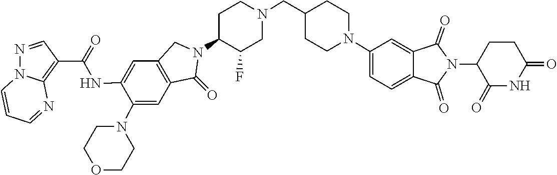 [SMILES: O=C1CCC(N2C(=O)c3ccc(N4CCC(CN5CC[C@H](N6Cc7cc(NC(=O)c8cnn9cccnc89)c(N8CCOCC8)cc7C6=O)[C@@H](F)C5)CC4)cc3C2=O)C(=O)N1] | 833.2 | DMSO-d₆ δ = 11.09 (s, 1 H), 10.92 (s, 1 H), 9.42 (dd, J = 1.6, 7.2 Hz, 1 H), 9.01 (dd, J = 1.6, 4.2 Hz, 1 H), 8.77 (d, J = 7.6 Hz, 2 H), 7.66 (d, J = 8.8 Hz, 1 H), 7.60 (s, 1 H), 7.42-7.37 (m, 1 H), 7.32 (s, 1 H), 7.25 (br d, J = 8.8 Hz, 1 H), 5.07 (dd, J = 5.6, 12.8 Hz, 1 H), 4.98-4.73 (m, 1 H), 4.61 -4.49 (m, 2 H), 4.22-4.00 (m, 3 H), 3.90 (br d, |
| Example 94 | | J = 4.8 Hz, 4 H), 3.08-2.80 (m, |
| | | 8 H), 2.64-2.53 (m, 2 H), 2.28 |
| | | (br d, J = 5.2 Hz, 2 H), 2.17-1.96 |
| | | (m, 3 H), 1.93-1.66 (m, 5 H), |
| | | 1.17(br d, J = 11.6 Hz, 2H) |
| 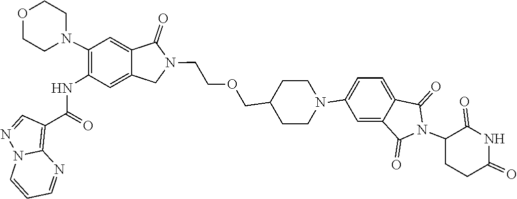 [SMILES: O=C1CCC(N2C(=O)c3ccc(N4CCC(COCCN5Cc6cc(NC(=O)c7cnn8cccnc78)c(N7CCOCC7)cc6C5=O)CC4)cc3C2=O)C(=O)N1] | 776.3 | CDCl3 δ = 10.94 (s, 1 H), 8.90- 8.86 (m, 2 H), 8.85-8.81 (m, 2 H), 8.02 (br s, 1 H), 7.74 (s, 1 H), 7.67 (d, J = 8.8 Hz, 1 H), 7.13 (dd, J = 4.2, 7.2 Hz, 1 H), 7.05 (dd, J = 2.4, 8.8 Hz, 1 H), 4.93 (dd, J = 5.2, 12.4 Hz, 1H), 4.52 (s, 2 H), 4.05-4.00 (m, 4 H), 3.96 (br d, J = 12.8 Hz, 2 H), 3.84-3.78 (m, 2 H), 3.73- 3.66 (m, 2 H), 3.34 (d, J = 6.0 Hz, 2 H), 3.04-2.67 (m, 10 H), 2.17-2.09 (m, 1 H), 1.83 (br s, |
| Example 95 | | 2H), 1.37-1.31 (m, 2H) |
| 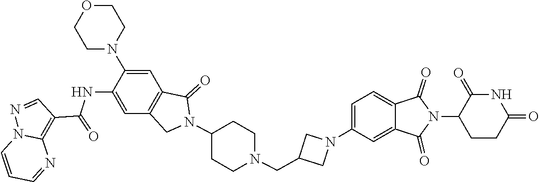 [SMILES: O=C1CCC(N2C(=O)c3ccc(N4CC(CN5CCC(N6Cc7cc(NC(=O)c8cnn9cccnc89)c(N8CCOCC8)cc7C6=O)CC5)C4)cc3C2=O)C(=O)N1] | 787.5 | DMSO-d₆ δ = 10.93 (s, 1 H) 8.86-8.90 (m, 2 H) 8.82-8.85 (m, 2 H) 8.19-8.22 (m, 1 H) 7.74 (s, 1 H) 7.66 (d, J =8.0 Hz, 1 H) 7.13 (dd, J = 6.8, 4.0 Hz, 1 H) 6.81 (d, J = 2.0 Hz, 1 H) 6.52 (dd, J = 8.4, 2.0 Hz, 1 H) 4.95 (dd, J = 12.0, 5.2 Hz, 1 H) 4.37 (s, 2 H) 4.24-4.34 (m, 1 H) 4.14- 4.21 (m, 2 H) 3.99-4.05 (m, 4 H) 3.70-3.77 (m, 2 H) 2.98-3.05 (m, 6 H) 2.81-2.92 (m, 2 H) 2.72 (br |
| Example 96 | | d, J = 7.34 Hz, 2H) 2.19-2.31 |
| | | (m, 2H) 2.10-2.18 (m, 1 H) 1.80- |
| | | 1.90 (m, 4 H) 1.22-1.34 (m, |
| | | 1 H) 0.85-0.88 (m, 1 H) |
| 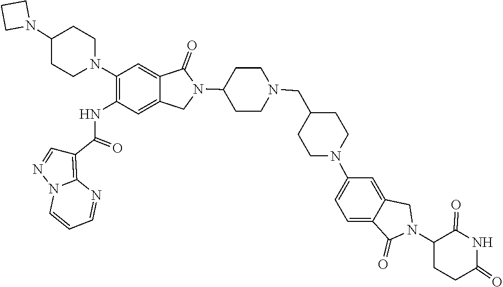 [SMILES: O=C1CCC(N2Cc3cc(N4CCC(CN5CCC(N6Cc7cc(NC(=O)c8cnn9cccnc89)c(N8CCC(N9CCC9)CC8)cc7C6=O)CC5)CC4)ccc3C2=O)C(=O)N1] | 854.5 | DMSO-d₆ δ = 10.88-11.04 (m, 2 H) 9.41 (dd, J = 1.6, 6.8 Hz, 1 H) 8.93 (dd, J = 1.2, 4.0 Hz, 1 H) 8.70-8.80 (m, 2 H) 7.42- 7.59 (m, 3 H) 7.00-7.09 (m, 2 H) 5.04 (dd, J = 4.8, 13.2 Hz, 1 H) 4.45 (s, 2 H) 4.34 (d, J = 16.8 Hz, 1 H) 4.21 (d, J = 16.8 Hz, 1 H) 3.95-4.05 (m, 1 H) 3.83-3.92 (m, 2 H) 3.12-3.20 (m, 4 H) 2.78-2.88 (m, 4 H) 2.69-2.77 (m, 5 H) 2.30-2.36 (m, 2 H) 1.92-2.08 (m, 6 H) 1.80 (m, 7 H) 1.51-1.74 (m, 5 H) 1.13-1.27 (m, 3 H) |
| Example 97 | | |
| 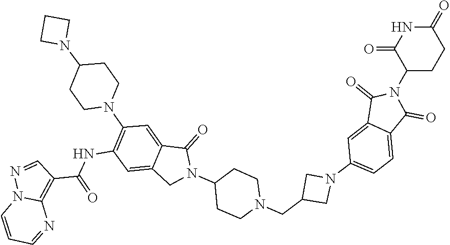 [SMILES: O=C1CCC(N2C(=O)c3ccc(N4CC(CN5CCC(N6Cc7cc(NC(=O)c8cnn9cccnc89)c(N8CCC(N9CCC9)CC8)cc7C6=O)CC5)C4)cc3C2=O)C(=O)N1] | 840.5 | DMSO-d₆ δ = 11.03-11.10 (m, 1 H) 10.95-11.00 (m, 1 H) 9.41 (dd, J = 1.6, 7.2 Hz, 1 H) 8.94 (dd, J = 1.2, 4.0 Hz, 1 H) 8.72- 8.80 (m, 2 H) 8.24 (m, 1 H) 7.64 (d, J = 8.4 Hz, 1 H) 7.54 (s, 1 H) 7.45 (dd, J = 4.4, 7.2 Hz, 1 H) 6.78 (d, J = 1.6 Hz, 1 H) 6.62- 6.69 (dd, J = 2.0, 8.0 Hz, 1 H) 5.00-5.10 (m, 1 H) 4.40-4.50 (m, 2 H) 4.11-4.20 (m, 2 H) 3.92-4.06 (m, 1 H) 3.65-3.75 (m, 2 H) 3.16-3.22 (m, 5 H) 2.93-3.05 (m, 5 H) 2.82-2.91 (m, 1 H) 2.70-2.78 (m, 2 H) |
| Example 98 | | 2.53-2.66 (m, 3 H) 2.21-2.31 |
| | | (m, 1 H) 2.05-2.16 (m, 2 H) |
| | | 1.95-2.04 (m, 3 H) 1.74-1.88 |
| | | (m, 4H) 1.58-1.73 (m, 4 H) |
| 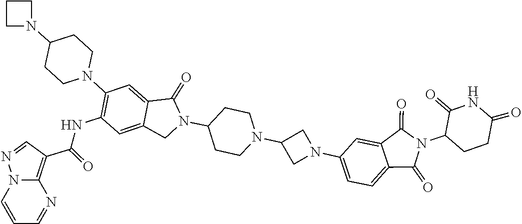 [SMILES: O=C1CCC(N2C(=O)c3ccc(N4CC(N5CCC(N6Cc7cc(NC(=O)c8cnn9cccnc89)c(N8CCC(N9CCC9)CC8)cc7C6=O)CC5)C4)cc3C2=O)C(=O)N1] | 826.5 | CDCl3 δ = 10.88 (s, 1 H) 9.23 (s, 1H) 8.86 (s, 1H) 8.82 (dd, J = 1.6, 7.2 Hz, 1H) 8.77 (s, 1 H) 8.71 (s, 1 H) 7.93-8.12 (m, 1 H) 7.71 (s, 1 H) 7.66 (d, J = 8.4 Hz, 1 H) 7.13 (dd, J = 4.4, 6.8 Hz, 1 H) 6.81 (d, J = 2.0 Hz, 1 H) 6.55 (dd, J = 2.0, 8.0 Hz, 1 H) 4.94 (dd, J = 5.2, 12 Hz, 1 H) 4.36 (s, 3 H) 4.15 (m, 2 H) 3.91 (dd, J = 5.2, 8.0 Hz, 2 H) 3.72 (m, 3 H) 3.43 (m, 1 H) 3.20 (m, 2 H) 3.02 (m, 2 H) 2.74- 2.94 (m, 5 H) 2.54-2.61 (m, 1 |
| Example 99 | | H) 2.32-2.37 (m, 2 H) 2.11-2.21 |
| | | (m, 5 H) 1.85-1.96 (m, 8 H) |
| 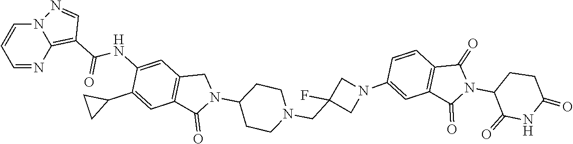 [SMILES: O=C1CCC(N2C(=O)c3ccc(N4CC(F)(CN5CCC(N6Cc7cc(NC(=O)c8cnn9cccnc89)c(C8CC8)cc7C6=O)CC5)C4)cc3C2=O)C(=O)N1] | 760.3 | DMSO-d₆ δ = 11.07 (s, 1H), 10.59 (s, 1H), 9.40 (dd, J = 1.2, 7.2 Hz, 1H), 8.91 (dd, J = 1.6, 4.0 Hz, 1H), 8.77 (s, 1H), 8.69 (s, 1H), 7.68 (d, J = 8.0 Hz, 1H), 7.45 (s, 1H), 7.36 (dd, J = 4.4, 7.2 Hz, 1H), 6.92 (d, J = 2.0 Hz, 1H), 6.78 (dd, J = 2.0, 8.0 Hz, 1H), 5.08 (m, 1H), 4.42 |
| Example 100 | | (s, 2H), 4.28-4.11 (m, 4H), |
| | | 4.06-3.95 (m, 1H), 3.04-3.01 |
| | | (m, 2H), 2.94-2.88 (m, 3H), |
| | | 2.56-2.55 (m, 2H), 2.32-2.31 |
| | | (m, 2H), 2.07-1.99 (m, 2H), |
| | | 1.80-1.79 (m, 2H), 1.68-1.67 |
| | | (m, 2H), 1.23-1.20 (m, 2H), |
| | | 0.78-0.76 (m, 2H) |
| 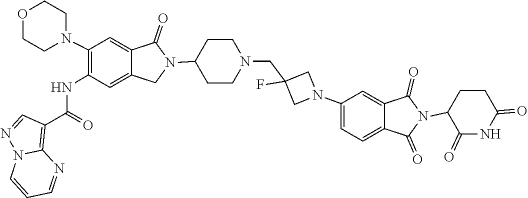 [SMILES: O=C1CCC(N2C(=O)c3ccc(N4CC(F)(CN5CCC(N6Cc7cc(NC(=O)c8cnn9cccnc89)c(N8CCOCC8)cc7C6=O)CC5)C4)cc3C2=O)C(=O)N1] | 805.1 | DMSO-d₆ δ = 11.06 (br s, 1H), 10.89 (s, 1H), 9.41 (dd, J = 1.6, 7.2 Hz, 1H), 8.99 (dd, J = 1.6, 4.0 Hz, 1H), 8.79-8.71 (m, 2H), 8.29 (s, 1H), 7.69 (d, J = 8.4 Hz, 1H), 7.57 (s, 1H), 7.39 (dd, J = 4.0, 6.8 Hz, 1H), 6.92 (d, J = 2.0 Hz, 1H), 6.78 (dd, J = 2.0, 8.4 Hz, 1H), 5.08 (dd, J = 5.6, 12.8 Hz, 1H), 4.44 (s, 2H), 4.28-4.11 (m, 4H), 4.05-3.96 (m, 1H), 3.94-3.85 (m, 4H), 3.03 (br d, J = 11.2 Hz, 2H), |
| Example 101 | | 2.96-2.84 (m, 7H), 2.62-2.56 |
| | | (m, 2H), 2.37-2.30 (m, 2H), |
| | | 2.06-1.99 (m, 1H), 1.88-1.76 |
| | | (m, 2H), 1.72-1.63 (m, 2H) |
| 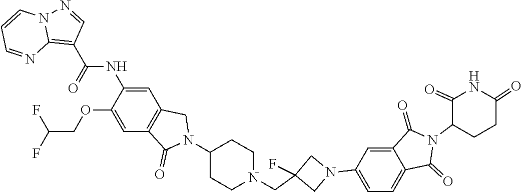 [SMILES: O=C1CCC(N2C(=O)c3ccc(N4CC(F)(CN5CCC(N6Cc7cc(NC(=O)c8cnn9cccnc89)c(OCC(F)F)cc7C6=O)CC5)C4)cc3C2=O)C(=O)N1] | 800.3 | DMSO-d₆ δ = 11.08 (s, 1 H), 10.78 (s, 1 H), 9.41 (d, J = 7.2 Hz, 1 H), 8.87 (d, J = 4.0 Hz, 1 H), 8.76 (d, J = 6.0 Hz, 2 H), 7.69 (d, J = 8.4 Hz, 1 H), 7.42 (s, 1 H), 7.38 (dd, J = 6.8, 4.4 Hz, 1 H), 6.92 (s, 1 H), 6.78 (d, J = 8.4 Hz, 1 H), 6.44-6.75 (m, 1H), 5.08 (dd, J = 12.8, 5.2 Hz, 1 H), 4.54-4.67 (m, 2 H), 4.44 (s, 2 H), 4.11-4.29 (m, 4 H), 4.00 (t, J = 11.6 Hz, 1 H), 3.03 |
| Example 102 | | (d, J = 10.8 Hz, 2 H), 2.83-2.96 |
| | | (m, 3 H), 2.55-2.63 (m, 1 H), |
| | | 2.55-2.63 (m, 1 H), 2.33 (t, J = |
| | | 11.2 Hz, 2 H), 1.97-2.07 (m, 1 |
| | | H), 1.76-1.87 (m, 2 H), 1.68 (d, |
| | | J = 10.4 Hz, 2 H) |
| 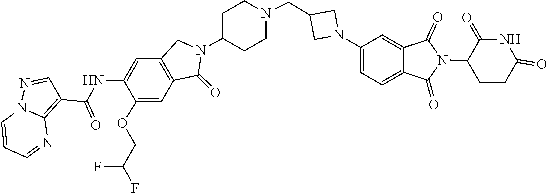 [SMILES: O=C1CCC(N2C(=O)c3ccc(N4CC(CN5CCC(N6Cc7cc(NC(=O)c8cnn9cccnc89)c(OCC(F)F)cc7C6=O)CC5)C4)cc3C2=O)C(=O)N1] | 782.2 | DMSO-d₆ δ = 11.09 (s, 1 H), 10.78 (s, 1 H), 9.39 (dd, J = 6.8, 1.6 Hz, 1 H), 8.86 (dd, J = 4.4, 1.6 Hz, 1 H), 8.75 (d, J = 10.4 Hz, 2 H), 7.62 (d, J = 8.0 Hz, 1 H), 7.42 (s, 1 H), 7.37 (dd, J = 6.8, 4.4 Hz, 1 H), 6.76 (d, J = 2.0 Hz, 1 H), 6.45-6.74 (m, 2 H), 5.06 (dd, J = 12.8, 5.2 Hz, 1 H), 4.60 (td, J = 14.4, 3.2 Hz, 2 H), 4.44 (s, 2H), 4.13 (t, J = 8.0 Hz, 2H), 3.99 (t, J = 11.6 Hz, 1 |
| Example 103 | | H), 3.63-3.73 (m, 2 H), 2.82- |
| | | 3.04 (m, 4 H), 2.52-2.66 (m, 4 |
| | | H), 1.98-2.14 (m, 3 H), 1.74- |
| | | 1.85 (m, 2H), 1.70 (s, 2 H) |
| 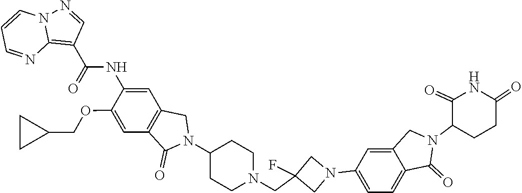 [SMILES: O=C1CCC(N2Cc3cc(N4CC(F)(CN5CCC(N6Cc7cc(NC(=O)c8cnn9cccnc89)c(OCC8CC8)cc7C6=O)CC5)C4)ccc3C2=O)C(=O)N1] | 776.3 | DMSO-d₆ δ = 10.96 (s, 1H), 10.84 (s, 1H), 9.41 (dd, J = 1.6, 7.2 Hz, 1H), 8.90 (dd, J = 1.6, 4.4 Hz, 1H), 8.75 (d, J = 4.0 Hz, 2H), 7.54 (d, J = 8.4 Hz, 1H), 7.35 (dd, J = 4.4, 7.2 Hz, 1H), 7.27 (s, 1H), 6.65 (s, 1H), 6.60 (dd, J = 1.6, 8.4 Hz, 1H), 5.07 (dd, J = 5.2, 13.6 Hz, 1H),4.41 (s, 2H), 4.37-4.30 (m, 1H), 4.24-4.18 (m, 1H), 4.16-4.04 (m, 5H), 4.04-3.98 (m, 2H), |
| Example 104 | | 3.03 (br d, J = 10.0 Hz, 2H), |
| | | 2.95-2.87 (m, 3H), 2.62-2.57 |
| | | (m, 2H), 2.36-2.31 (m, 2H), |
| | | 2.01-1.93 (m, 1H), 1.86-1.76 |
| | | (m, 2H), 1.72-1.64 (m, 2H), |
| | | 1.51-1.43 (m, 1H), 0.73-0.67 |
| | | (m, 2H), 0.48-0.43 (m, 2H) |
| 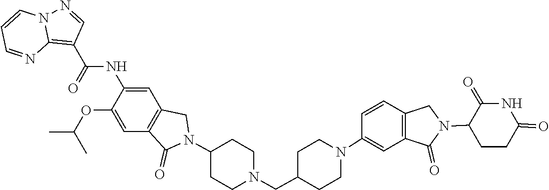 [SMILES: CC(C)Oc1cc2c(cc1NC(=O)c1cnn3cccnc13)CN(C1CCN(CC3CCN(c4ccc5c(c4)C(=O)N(C4CCC(=O)NC4=O)C5)CC3)CC1)C2=O] | 774.3 | DMSO-d₆ δ = 10.97 (br s, 1H), 10.76 (s, 1H), 9.41 (dd, J = 1.6, 7.2 Hz, 1H), 8.90 (dd, J = 1.6, 4.4 Hz, 1H), 8.75 (s, 2H), 8.21 (br s, 1H), 7.42 (d, J = 8.8 Hz, 1H), 7.37 (dd, J = 4.4, 7.2 Hz, 1H), 7.34 (s, 1H), 7.29-7.24 (m, 1H), 7.16 (d, J = 2.0 Hz, 1H), 5.10 (dd, J = 5.2, 13.6 Hz, 1H), 4.91 (td, J = 6.0, 12.0 Hz, 1H), 4.44 (s, 2H), 4.36-4.19 (m, 2H), 4.04-3.96 (m, 1H), 3.77 (br d, J = |
| Example 105 | | 11.6 Hz, 2H), 2.97 (br d, J = |
| | | 11.6 Hz, 2H), 2.93-2.87 (m, |
| | | 1H), 2.73 (br t, J = 11.2 Hz, |
| | | 2H), 2.60 (br d, J = 18.0 Hz, |
| | | 1H), 2.39 (br dd, J = 4.8, 12.8 |
| | | Hz, 1H), 2.21 (br d, J = 7.2 Hz, |
| | | 2H), 2.06-1.97 (m, 3H), 1.82 |
| | | (br d, J = 12.0 Hz, 4H), 1.74- |
| | | 1.67 (m, 3H), 1.45 (d, J = 6.0 |
| | | Hz, 6H), 1.30-1.19 (m, 2H) |
| 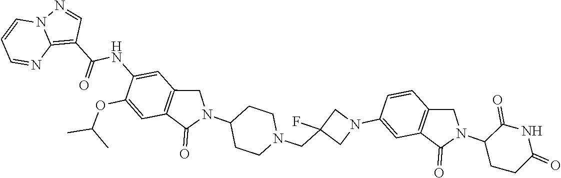 [SMILES: CC(C)Oc1cc2c(cc1NC(=O)c1cnn3cccnc13)CN(C1CCN(CC3(F)CN(c4ccc5c(c4)C(=O)N(C4CCC(=O)NC4=O)C5)C3)CC1)C2=O] | 764.3 | DMSO-d₆ δ = 10.96 (s, 1 H) 10.75 (s, 1 H) 9.40 (dd, J = 6.80, 1.60 Hz, 1 H) 8.89 (dd, J = 4.00, 1.60 Hz, 1H) 8.74 (s, 2 H) 7.42 (d, J = 8.80 Hz, 1 H) 7.32-7.38 (m, 2 H) 6.78-6.82 (m, 2 H) 5.10 (dd, J = 13.20, 5.20 Hz, 1 H) 4.85-4.94 (m, 1 H) 4.31- 4.43 (m, 3 H) 4.17-4.26 (m, 1 H) 4.07 (dd, J = 18.00, 9.20 Hz, 2 H) 3.98 (br d, J = 9.20 Hz, 2 H) |
| Example 106 | | 3.89-3.95 (m, 1 H) 3.03 (br d, |
| | | J = 10.80 Hz, 2 H) 2.85-2.94 |
| | | (m, 3 H) 2.59 (br dd, J = 15.60, |
| | | 1.88 Hz, 2 H) 2.33-2.42 (m, 2 |
| | | H) 1.95-2.05 (m, 1 H) 1.77-1.86 |
| | | (m, 2 H) 1.68 (br d, J = 9.20 Hz, |
| | | 2 H) 1.44 (d, J = 6.00 Hz, 6 H) |
| 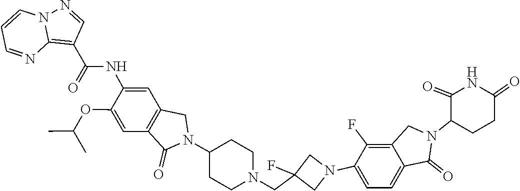 [SMILES: CC(C)Oc1cc2c(cc1NC(=O)c1cnn3cccnc13)CN(C1CCN(CC3(F)CN(c4ccc5c(c4F)CN(C4CCC(=O)NC4=O)C5=O)C3)CC1)C2=O] | 782.1 | DMSO-d₆ δ = 10.97 (s, 1 H) 10.74 (s, 1 H) 9.40 (dd, J = 6.80, 0.8 Hz, 1 H) 8.89 (dd, J = 4.00, 2.8 Hz, 1 H) 8.73 (s, 2 H) 7.42 (d, J = 8.40 Hz, 1 H) 7.34-7.37 (m, 1 H) 7.33 (s, 1 H) 6.73 (t, J = 8.0 Hz, 1 H) 5.06-5.05 (m, 1 H) 4.89-4.87 (m, 1 H) 4.49-4.44 (m, 1 H) 4.44-4.33 (m, 2 H) 4.32- 4.27 (m, 5 H) 4.17-4.12 (m, 1 H) 3.03-3.01 (m, 2 H) 2.94-2.88 (m, 3 H) 2.54-2.53 (m, 1 H) 2.48-2.47 |
| Example 107 | | (m, 1 H) 2.31-2.30 (m, 2 H) 2.01- |
| | | 2.0 (m, 1 H) 1.80-1.70 (m, 2 H) |
| | | 1.69-1.66 (m, 2 H) 1.44 (d, J = |
| | | 6.00 Hz, 6 H) |
| 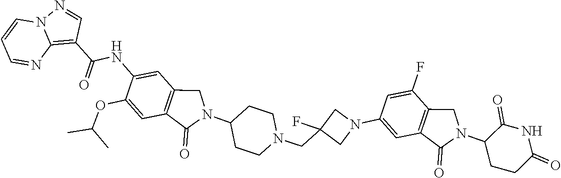 [SMILES: CC(C)Oc1cc2c(cc1NC(=O)c1cnn3cccnc13)CN(C1CCN(CC3(F)CN(c4cc(F)c5c(c4)C(=O)N(C4CCC(=O)NC4=O)C5)C3)CC1)C2=O] | 782.2 | DMSO-d₆ δ = 10.99 (s, 1 H), 10.75 (s, 1 H), 9.40 (m, 1 H), 8.89 (m, 1 H), 8.74 (s, 2 H), 7.30-7.39 (m, 2 H), 6.61-6.70 (m, 2 H), 5.10 (m, 1 H), 4.89 (m, 1 H), 4.38-4.47 (m, 3 H), 4.26 (d, J = 16.8 Hz, 1 H), 4.09 (m, 2 H), 3.94-4.03 (m, 3 H), 3.02 (d, J = 10.4 Hz, 2 H), 2.91- 2.94 (m, 1 H), 2.86 (s, 1 H), 2.54-2.64 (m, 2 H), 2.42 (m, 1 |
| Example 108 | | H), 2.32 (t, J = 11.2 Hz, 2 H), |
| | | 1.95-2.04 (m, 1 H), 1.75-1.88 |
| | | (m, 2 H), 1.68 (d, J = 10.0 Hz, |
| | | 2H), 1.44 (d, J = 6.0 Hz, 6 H) |
| 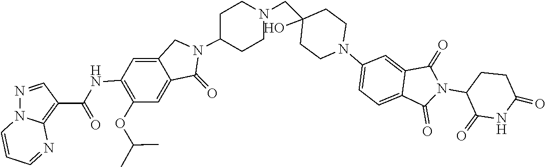 [SMILES: CC(C)Oc1cc2c(cc1NC(=O)c1cnn3cccnc13)CN(C1CCN(CC3(O)CCN(c4ccc5c(c4)C(=O)N(C4CCC(=O)NC4=O)C5=O)CC3)CC1)C2=O] | 804.2 | DMSO-d₆ δ = 11.08 (s, 1H), 10.75 (s, 1H), 9.41 (dd, J = 1.4, 7.0 Hz, 1H), 8.89 (dd, J = 1.6, 4.2 Hz, 1H), 8.73 (d, J = 2.6 Hz, 2H), 7.66 (d, J = 8.6 Hz, 1H), 7.38-7.31 (m, 3H), 7.26 (dd, J = 1.8, 8.6 Hz, 1H), 5.09 (dd, J = 5.4, 12.8 Hz, 1H), 4.89 (td, J = 6.0, 12.0 Hz, 1H), 4.39 (s, 2H), 4.01-3.90 (m, 1H), 3.79 (br d, J = 12.0 Hz, 2H), 3.03 (br d, J = |
| Example 109 | | 11.4 Hz, 2H), 2.95-2.83 (m, |
| | | 1H), 2.63 -2.52 (m,5H), 2.36- |
| | | 2.25 (m, 4H), 2.07-1.97 (m, |
| | | 1H), 1.84-1.72 (m, 2H), 1.68-1.52 |
| | | (m, 6H), 1.43 (d, J = 6.0 Hz, 6H) |
| 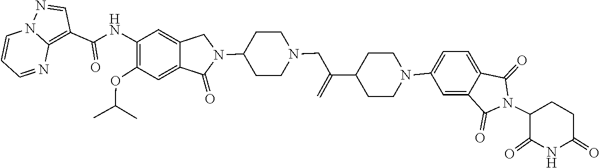 [SMILES: C=C(CN1CCC(N2Cc3cc(NC(=O)c4cnn5cccnc45)c(OC(C)C)cc3C2=O)CC1)C1CCN(c2ccc3c(c2)C(=O)N(C2CCC(=O)NC2=O)C3=O)CC1] | 814.3 | DMSO-d₆ δ = 11.06 (s, 1 H), 10.75 (s, 1 H), 9.40 (m, 1 H), 8.89 (m, 1 H), 8.74 (d, J = 2.0 Hz, 2 H), 7.67 (d, J = 8.4 Hz, 1 H), 7.31-7.39 (m, 3 H), 7.27 (m, 1 H), 5.09 (m, 1 H), 4.96 (s, 1 H), 4.85-4.92 (m, 2 H), 4.40 (s, 2 H), 4.14 (d, J = 12.0 Hz, 2H), 3.92-4.04 (m, 1 H), 2.85-3.05 (m, 7 H), 2.56 (s, 3 H), 1.93-2.05 (m, 3 H), 1.65-1.87 (m, 6 H), |
| Example 110 | | 1.46-1.55 (m, 2H), 1.44 (d, |
| | | J = 6.0 Hz, 6 H) |
| 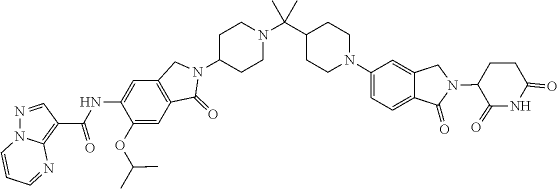 [SMILES: CC(C)Oc1cc2c(cc1NC(=O)c1cnn3cccnc13)CN(C1CCN(C(C)(C)C3CCN(c4ccc5c(c4)CN(C4CCC(=O)NC4=O)C5=O)CC3)CC1)C2=O] | 802.3 | DMSO-d₆ δ = 10.95 (s, 1H), 10.75 (s, 1H), 9.40 (dd, J = 1.6, 7.2 Hz, 1H), 8.89 (dd, J = 2.0, 4.4 Hz, 1H), 8.76-8.71 (m, 2H), 7.50 (d, J = 9.2 Hz, 1H), 7.39-7.31 (m, 2H), 7.08-7.02 (m, 2H), 5.05 (dd, J = 4.8, 13.2 Hz, 1H), 4.90 (td, J = 6.0, 12.0 Hz, 1H), 4.46- 4.39 (m, 2H), 4.36-4.16 (m, 2H), 3.97 (br d, J =10.8 Hz, 3H), 3.11 (br d, J = 10.0 Hz, 2H), 2.97-2.84 (m, 1H), 2.76 (br t, J = 12.0 Hz, |
| Example 111 | | 2H), 2.62-2.53 (m, 2H), 2.36- |
| | | 2.30 (m, 1H), 2.22 (br s, 2H), |
| | | 1.85-1.68 (m, 7H), 1.44 (d, J = |
| | | 6.0 Hz, 6H), 1.38-1.23 (m, |
| | | 2H), 0.93 (s, 6H) |
| 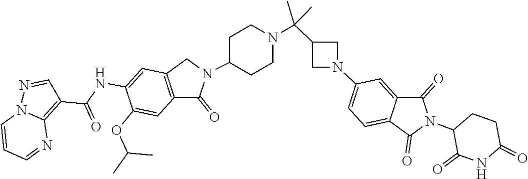 [SMILES: CC(C)Oc1cc2c(cc1NC(=O)c1cnn3cccnc13)CN(C1CCN(C(C)(C)C3CN(c4ccc5c(c4)C(=O)N(C4CCC(=O)NC4=O)C5=O)C3)CC1)C2=O] | 788.3 | DMSO-d₆ δ = 11.08 (s, 1H), 10.77 (s, 1H), 9.41 (dd, J = 1.6, 7.2 Hz, 1H), 8.90 (dd, J = 1.6, 4.4 Hz, 1H), 8.79-8.70 (m, 2H), 7.65 (d, J = 8.4 Hz, 1H), 7.37 (dd, J = 4.4, 7.2 Hz, 1H), 7.34 (s, 1H), 6.79 (d, J = 2.0 Hz, 1H), 6.66 (dd, J = 2.0, 8.4 Hz, 1H), 5.06 (dd, J = 5.2, 12.8 Hz, 1H), 4.91 (td, J = 6.0, 12.0 Hz, 1H), 4.42 (s, 2H), 4.11-4.05 (m, 2H), 4.05-3.97 (m, 1H), 3.87 (br t, J = |
| Example 112 | | 7.2 Hz, 2H), 3.20-3.02 (m, 3H), |
| | | 2.94-2.83 (m, 1H), 2.63-2.57 (m, |
| | | 1H), 2.57-2.53 (m, 2H), 2.28-2.17 |
| | | (m, 1H), 2.06-1.98 (m, 1H), 1.81- |
| | | 1.67 (m, 4H), 1.45 (d, J = 6.0 Hz, |
| | | 6H), 1.07 (br s, 6H) |
| 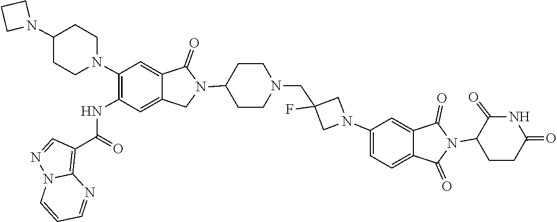 [SMILES: O=C1CCC(N2C(=O)c3ccc(N4CC(F)(CN5CCC(N6Cc7cc(NC(=O)c8cnn9cccnc89)c(N8CCC(N9CCC9)CC8)cc7C6=O)CC5)C4)cc3C2=O)C(=O)N1] | 858.4 | DMSO-d₆ δ = 11.07 (s, 1H), 10.99 (s, 1H), 9.41 (dd, J = 1.2, 7.2 Hz, 1H), 8.93 (d, J = 3.2 Hz, 1H), 8.76 (d, J = 7.2 Hz, 2H), 7.69 (d, J = 8.4 Hz, 1H),7.54 (s, 1H), 7.46 (dd, J = 4.0, 6.8 Hz, 1H), 6.92 (d, J = 1.2 Hz, 1H), 6.78 (dd, J = 2.0, 8.4 Hz, 1H), 5.08 (dd, J = 4.8, 12.4 Hz, 1H), 4.43 (s, 2H), 4.27-4.12 (m, 4H), 4.05-3.95 (m, 1H), 3.18- 3.14 (m, 3H), 3.07-2.92 (m, 6H), 2.90-2.83 (m, 2H), 2.78- 2.71 (m, 2H), 2.64-2.56 (m, |
| Example 116 | | 2H), 2.37-2.29 (m, 3H), 2.05- |
| | | 1.95 (m, 3H), 1.85-1.75 (m, |
| | | 4H), 1.69-1.58 (m, 4H) |
| 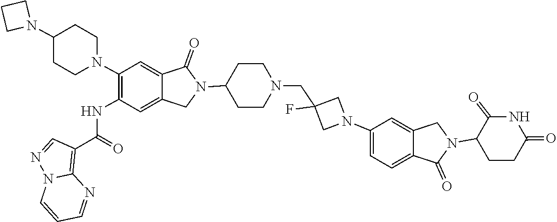 [SMILES: O=C1CCC(N2Cc3cc(N4CC(F)(CN5CCC(N6Cc7cc(NC(=O)c8cnn9cccnc89)c(N8CCC(N9CCC9)CC8)cc7C6=O)CC5)C4)ccc3C2=O)C(=O)N1] | 844.3 | DMSO-d₆ δ = 10.96 (d, J = 11.2 Hz, 2H), 9.41 (dd, J = 1.6, 7.2 Hz, 1H), 8.95 (dd, J = 1.6, 4.2 Hz, 1H), 8.82-8.70 (m, 2H), 8.19 (s, 1H), 7.57-7.49 (m, 2H), 7.45 (dd, J = 4.4, 6.8 Hz, 1H), 6.68-6.55 (m, 2H), 5.06 (dd, J = 5.2, 13.2 Hz, 1H), 4.43 (s, 2H), 4.37-4.29 (m, 1H), 4.26-3.94 (m, 7H), 3.08- 2.83 (m, 9H),2.73 (br t,J = 10.0 Hz, 3H), 2.36-2.25 (m, 4H), 2.09-1.90 (m, 4H), 1.88-1.74 (m, 4H), 1.70-1.56 (m, 4H) |
| Example 117 | | |

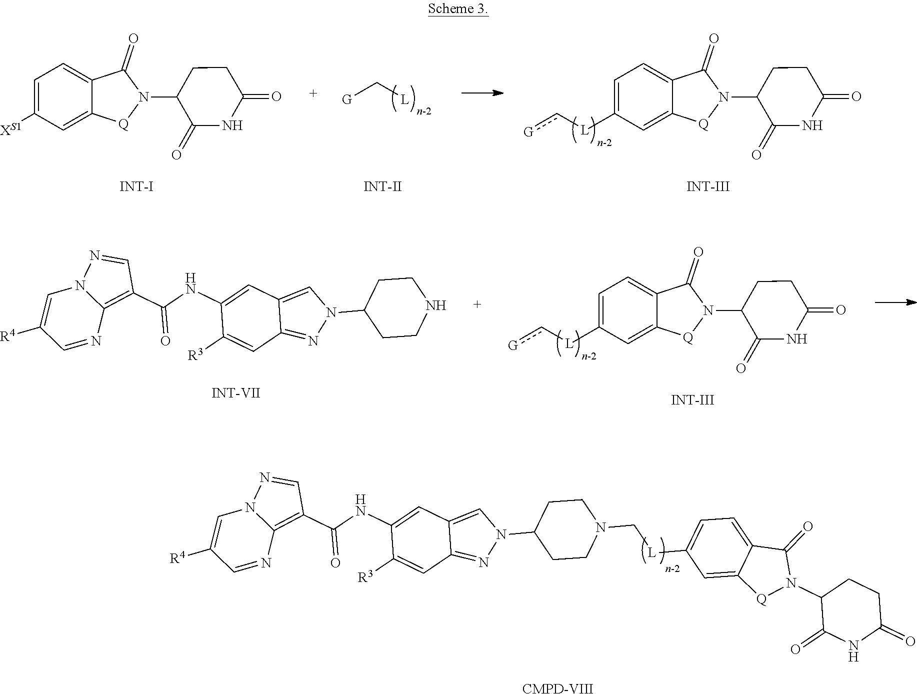

A compound of formula INT-I, where in X^{S1} is a halide or alcohol, may be reacted with a reagent INT-II (commercially available or readily prepared using standard reaction techniques known to one skilled in the art) under etherification or alkylation conditions to produce a compound of formula INT-III, wherein L and n are as defined herein, and Q is CH₂ or C═O. Compounds of formula INT-III may react with a compound of formula INT-VII, wherein R³ is an ether or alkyl group and R⁴ is optionally substituted as an H, amine, methyl, or chloro group, through N-alkylation where G is an appropriate leaving group (e.g., OMs, OTs, Cl, etc.) or through reductive amination where G is an aldehyde or ketone to produce compound of formula CNPD-VIII. When G is a leaving group, suitable reaction conditions are those for an alkylation reaction, e.g., diisopropylethylamine, potassium iodide, DMSO or acetonitrile, 80° C. When Gis a carbonyl, suitable reaction conditions are those for a reductive amination reaction, e.g., sodium cyanoborohydride, methanol, dichloromethane, acetic acid, room temperature.

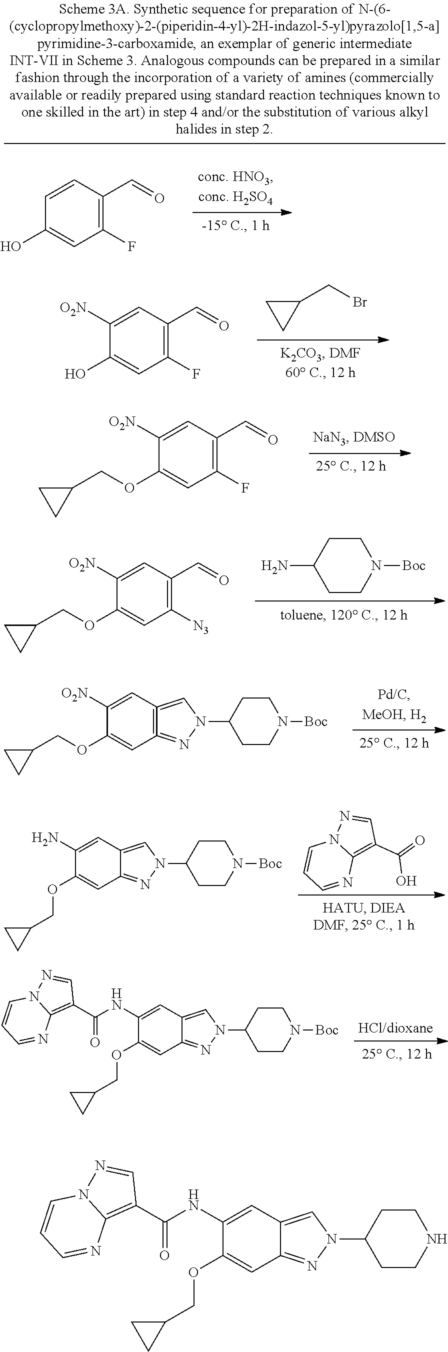

### Step 1:

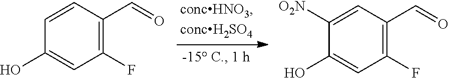 [SMILES: O=Cc1ccc(O)cc1F ; O=Cc1cc([N+](=O)[O-])c(O)cc1F]

To a solution of 2-fluoro-4-hydroxybenzaldehyde (10.0 g, 71.4 mmol, 1 eq) in sulfuric acid (60 mL) cooled to −15° C. was slowly added a mixture of nitric acid (6 mL) and sulfuric acid (14 mL). The mixture was stirred at −15° C. for 1 h then it was poured into water (500 mL) at 0° C. and extracted with ethyl acetate (200 mL×3). The combined organic layers were washed with brine (200 mL×3), dried over anhydrous sodium sulfate, concentrated in vacuo to give a residue. The crude product 2-fluoro-4-hydroxy-5-nitrobenzaldehyde (13.2 g, 71.3 mmol, >98% yield) was obtained as a yellow solid and used in subsequent transformations without further purification.

### Step 2:

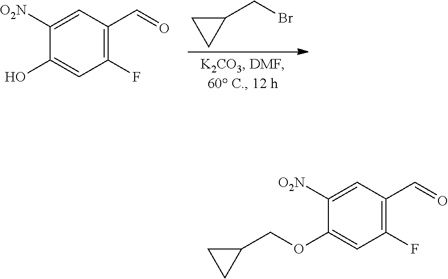

To a solution of 2-fluoro-4-hydroxy-5-nitrobenzaldehyde (15.0 g, 81.03 mmol, 1 eq) in N,N-dimethylformamide (100 mL) was added potassium carbonate (34 g, 246.0 mmol, 3.04 eq) and (bromomethyl)cyclopropane (15 g, 111.1 mmol, 10.64 mL, 1.37 eq). The mixture was stirred at 60° C. for 12 h. The reaction mixture was poured into water (300 mL) at 0° C., and then extracted with ethyl acetate (200 mL×3). The combined organic layers were washed with brine (200 mL×3), dried over anhydrous sodium sulfate, concentrated under reduced pressure to give a residue. The residue was purified by column chromatography (silicon dioxide, eluted with petroleum ether/ethyl acetate=20:1 to 3:1) to afford 4-(cyclopropylmethoxy)-2-fluoro-5-nitrobenzaldehyde (6.5 g, 27.17 mmol, 34% yield) as a yellow solid. MS (ESI) m/z: 240.1 [M+H]⁺.

### Step 3:

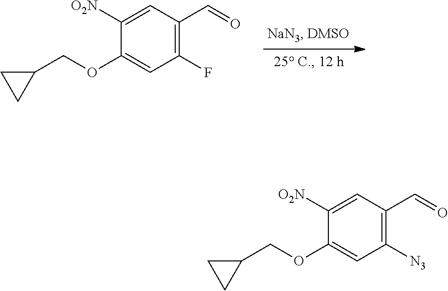 [SMILES: O=Cc1cc([N+](=O)[O-])c(OCC2CC2)cc1F ; [N-]=[N+]=Nc1cc(OCC2CC2)c([N+](=O)[O-])cc1C=O]

To a solution of 4-(cyclopropylmethoxy)-2-fluoro-5-nitrobenzaldehyde (6.5 g, 27.2 mmol, 1 eq) in dimethyl sulfoxide (40 mL) was added sodium azide (3.6 g, 55.4 mmol, 2.04 eq). The mixture was stirred at 25° C. for 12 h. The reaction mixture was poured into water (50 mL) at 0° C., and then extracted with ethyl acetate (30 mL×3). The combined organic layers were washed with brine (30 mL×3), dried over anhydrous sodium sulfate, and concentrated in vacuo to give a residue. The crude product 2-azido-4-(cyclopropylmethoxy)-5-nitrobenzaldehyde (7 g, 26.70 mmol, 98% yield) obtained as a yellow oil was used in the next step without further purification. MS (ESI) m/z: 263.1 [M+H]⁺.

### Step 4:

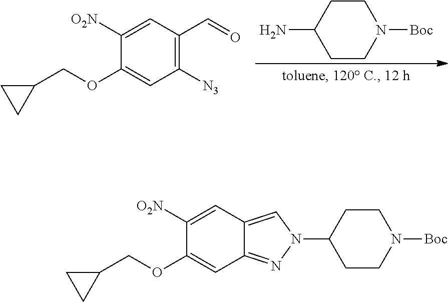

To a solution of 2-azido-4-(cyclopropylmethoxy)-5-nitrobenzaldehyde (7.0 g, 26.7 mmol, 1 eq) in toluene (70 mL) was added tert-butyl 4-aminopiperidine-1-carboxylate (5.5 g, 27.5 mmol, 1.03 eq). The mixture was stirred at 120° C. for 12 h. The reaction mixture was concentrated under reduced pressure. The residue was purified by column chromatography (silicon dioxide, eluted with petroleum ether/ethyl acetate=10:1 to 0.5:1) to afford tert-butyl 4-(6-(cyclopropylmethoxy)-5-nitro-2H-indazol-2-yl)piperidine-1-carboxylate (10.5 g, 25.21 mmol, 94% yield) as a yellow solid. MS (ESI) m/z: 417.2 [M+H]⁺.

### Step 5:

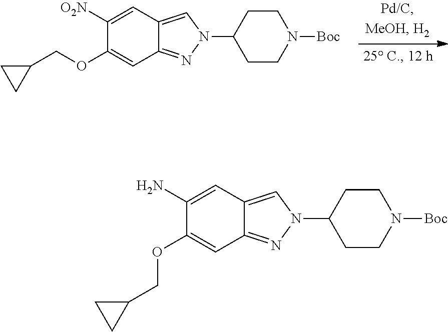 [SMILES: CC(C)(C)OC(=O)N1CCC(n2cc3cc([N+](=O)[O-])c(OCC4CC4)cc3n2)CC1 ; CC(C)(C)OC(=O)N1CCC(n2cc3cc(N)c(OCC4CC4)cc3n2)CC1]

To a solution of tert-butyl 4-(6-(cyclopropylmethoxy)-5-nitro-2H-indazol-2-yl)piperidine-1-carboxylate (10.5 g, 25.2 mmol, 1 eq) in methanol (150 mL) was added palladium on carbon (11.00 g, 10.34 mmol, 10% purity, 0.41 eq) under nitrogen atmosphere. The suspension was degassed under vacuum and purged with hydrogen several times. The mixture was stirred under hydrogen (15 psi) at 25° C. for 12 h. The reaction mixture was filtered, and the filtrate was concentrated. The residue was purified by preparative HPLC (column: Phenomenex Synergi Max-RP 250×80 mm×10 mm; mobile phase: [water (10 mM ammonium hydrogen carbonate)-acetonitrile]; B %: 40%-65%, 18 min) yielding tert-butyl 4-(5-amino-6-(cyclopropylmethoxy)-2H-indazol-2-yl)piperidine-1-carboxylate (7 g, 18.1 mmol, 72% yield) as a yellow solid. MS (ESI) m/z: 387.3 [M+H]⁺.

### Step 6:

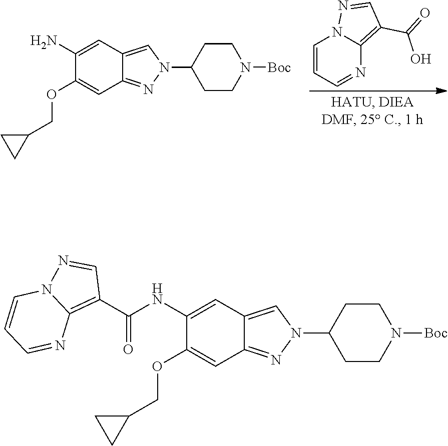

To a solution of tert-butyl 4-(5-amino-6-(cyclopropylmethoxy)-2H-indazol-2-yl)piperidine-1-carboxylate (500 mg, 1.29 mmol, 1 eq) and pyrazolo[1,5-a]pyrimidine-3-carboxylic acid (220 mg, 1.35 mmol, 1.04 eq) in N,N-dimethylformamide (5 mL) was added diisopropylethylamine (500 mg, 3.87 mmol, 673.8 μL, 2.99 eq), followed by the addition of HATU (740 mg, 1.95 mmol, 1.5 eq) to the mixture. The mixture was stirred at 25° C. for 1 h. The reaction mixture was poured into water (500 mL) at 0° C., and then extracted with ethyl acetate (250 mL×3). The combined organic layers were washed with brine (250 mL×3), dried over anhydrous sodium sulfate, concentrated in vacuo to give a residue. The residue was purified by column chromatography (silicon dioxide, eluted with petroleum ether/ethyl acetate=4:1 to 1:4) to afford tert-butyl 4-(6-(cyclopropylmethoxy)-5-(pyrazolo[1,5-a]pyrimidine-3-carboxamido)-2H-indazol-2-yl)piperidine-1-carboxylate (300 mg, 564.33 μmol, 44% yield) as a yellow solid. MS (ESI) m/z: 532.5 [M+H]⁺.

### Step 7:

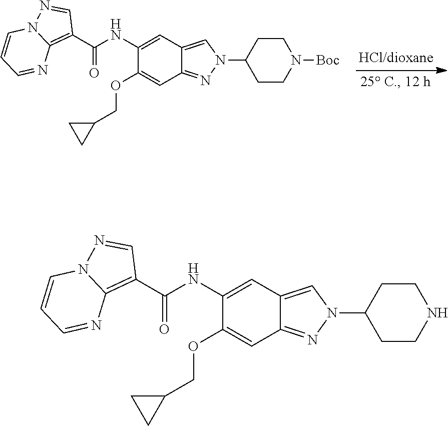 [SMILES: CC(C)(C)OC(=O)N1CCC(n2cc3cc(NC(=O)c4cnn5cccnc45)c(OCC4CC4)cc3n2)CC1 ; O=C(Nc1cc2cn(C3CCNCC3)nc2cc1OCC1CC1)c1cnn2cccnc12]

To a solution of tert-butyl 4-(6-(cyclopropylmethoxy)-5-(pyrazolo[1,5-a]pyrimidine-3-carboxamido)-2H-indazol-2-yl)piperidine-1-carboxylate (300 mg, 564.33 mmol, 1 eq) in dioxane (2 mL) was added a solution of hydrogen chloride in dioxane (4 M, 4 mL, 28.35 eq). The mixture was stirred at 25° C. for 12 h. The reaction mixture was concentrated under reduced pressure. The residue was diluted with tetrahydrofuran and the pH was adjusted to 7-8 with ammonium hydroxide. The mixture was then filtered, and the filtrate was concentrated under reduced pressure to give a residue. The crude product N-(6-(cyclopropylmethoxy)-2-(piperidin-4-yl)-2H-indazol-5-yl)pyrazolo[1,5-a]pyrimidine-3-carboxamide (240 mg, 556.21 mmol, 99% yield) obtained as a yellow solid was used in subsequent reactions without further purification. MS (ESI) m/z: 432.3 [M+H]⁺.

### Preparation of Example 118: N-(6-(cyclopropylmethoxy)-2-(1-((1-(2-(2,6-dioxopiperidin-3-yl)-1-oxoisoindolin-5-yl)piperidin-4-yl)methyl)piperidin-4-yl)-2H-indazol-5-yl)pyrazolo[1,5-a]pyrimidine-3-carboxamide

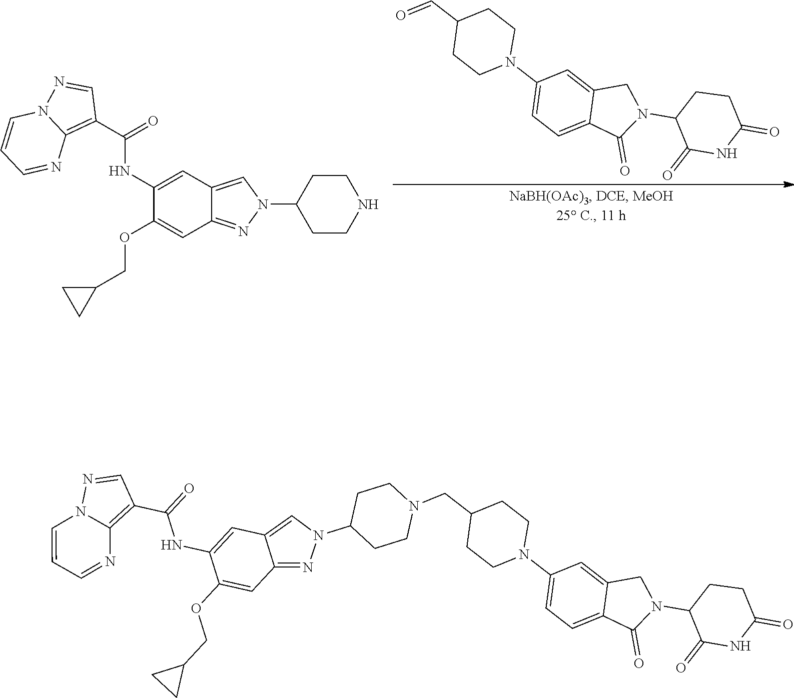 [SMILES: O=C(Nc1cc2cn(C3CCNCC3)nc2cc1OCC1CC1)c1cnn2cccnc12 ; O=CC1CCN(c2ccc3c(c2)CN(C2CCC(=O)NC2=O)C3=O)CC1 ; O=C1CCC(N2Cc3cc(N4CCC(CN5CCC(n6cc7cc(NC(=O)c8cnn9cccnc89)c(OCC8CC8)cc7n6)CC5)CC4)ccc3C2=O)C(=O)N1]

To a solution of N-(6-(cyclopropylmethoxy)-2-(piperidin-4-yl)-2H-indazol-5-yl)pyrazolo[1,5-a]pyrimidine-3-carboxamide (60 mg, 139 mmol, 1 eq) and 1-(2-(2,6-dioxopiperidin-3-yl)-1-oxoisoindolin-5-yl)piperidine-4-carbaldehyde (50 mg, 140.7 mmol, 1.01 eq) in 1,2-dichloroethane (2 mL) and methanol (0.5 mL) was added acetic acid (26.2 mg, 437 mmol, 25 mL, 3.14 eq). The reaction mixture was allowed to stir at 25° C. for 1 h prior to the addition of sodium triacetoxyhydroborate (120 mg, 566.2 mmol, 4.07 eq). The mixture was stirred at 25° C. for 10 h, after which it was poured into water (20 mL) at 0° C. The mixture was extracted with ethyl acetate (50 mL×3). The combined organic phases were washed with brine (50 mL), dried over anhydrous sodium sulfate, concentrated in vacuo to give a residue. The residue was purified by preparative HPLC (column: Phenomenex Luna C18 150×25 mm×10 mm; mobile phase: [water (0.1% trifluoroacetic acid)-acetonitrile]; B %: 18%-48%, 10 min) to afford N-(6-(cyclopropylmethoxy)-2-(1-((1-(2-(2,6-dioxopiperidin-3-yl)-1-oxoisoindolin-5-yl)piperidin-4-yl)methyl)piperidin-4-yl)-2H-indazol-5-yl)pyrazolo[1,5-a]pyrimidine-3-carboxamide (17 mg, 21.2 mmol, 15% yield, 96% purity) as a yellow solid. MS (ESI) m/z: 731.3 [M+H]⁺. ¹H NMR (400 MHz, methanol-d₄) δ=10.83 (s, 1H), 9.13 (dd, J=2.0, 7.2 Hz, 1H), 8.85 (dd, J=2.0, 4.4 Hz, 1H), 8.78 (s, 1H), 8.69 (s, 1H), 8.19 (s, 1H), 7.64 (d, J=9.2 Hz, 1H), 7.26 (dd, J=4.0, 7.2 Hz, 1H), 7.15-7.07 (m, 2H), 7.00 (s, 1H), 5.11 (dd, J=5.2, 12.8 Hz, 1H), 4.83-4.73 (m, 1H), 4.41 (d, J=6.4 Hz, 2H), 4.10-3.96 (m, 4H), 3.86 (br d, J=13.6 Hz, 2H), 3.64-3.56 (m, 1H), 3.29-3.24 (m, 1H), 3.22-3.15 (m, 2H), 3.02-2.85 (m, 3H), 2.80 (m, 1H), 2.58-2.39 (m, 5H), 2.16 (m, 2H), 1.95 (br d, J=12.8 Hz, 2H), 1.62-1.42 (m, 3H), 0.83-0.72 (m, 2H), 0.58-0.49 (m, 2H).

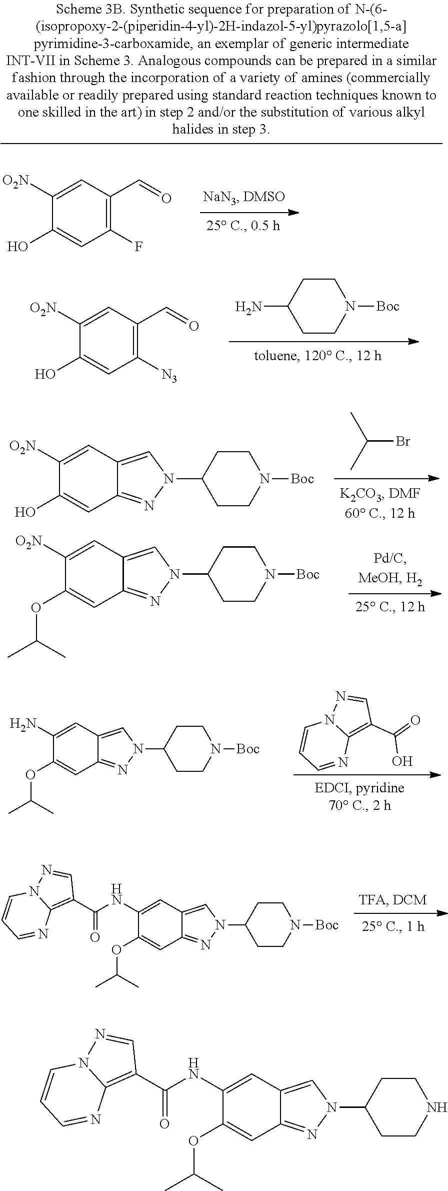

### Step 1:

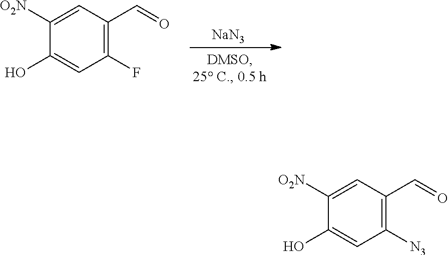 [SMILES: O=Cc1cc([N+](=O)[O-])c(O)cc1F ; [N-]=[N+]=Nc1cc(O)c([N+](=O)[O-])cc1C=O ; "NaN3"]

To a solution of 2-fluoro-4-hydroxy-5-nitrobenzaldehyde (2 g, 10.80 mmol, 1 eq) in dimethyl sulfoxide (130 mL) was added sodium azide (1.05 g, 16.21 mmol, 1.5 eq). The mixture was stirred at 25° C. for 0.5 h. The reaction mixture was partitioned between ethyl acetate (100 mL) and water (100 mL). The organic phase was separated, washed with brine (50 mL×2), dried over anhydrous sodium sulfate, filtered, and concentrated under reduced pressure to afford 2-azido-4-hydroxy-5-nitrobenzaldehyde (2.2 g, 10.57 mmol, 98% yield) as a yellow solid. ¹H NM/IR (400 MHz, CDCl₃) δ=11.12 (br s, 1H), 10.18 (s, 1H), 8.69 (s, 1H), 6.97 (s, 1H).

### Step 2:

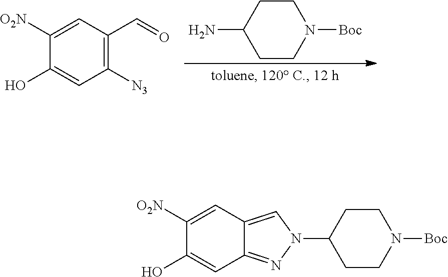

To a solution of tert-butyl 4-(6-hydroxy-5-nitro-2H-indazol-2-yl)piperidine-1-carboxylate (5 g, 24.0 mmol, 1 eq) and tert-butyl 4-aminopiperidine-1-carboxylate (4.81 g, 24.0 mmol, 1 eq) in toluene (100 mL) was added anhydrous sodium sulfate (16.65 g, 117.23 mmol, 11.89 mL, 4.88 eq). The mixture was stirred at 120° C. for 12 h. The reaction mixture was filtered and concentrated under reduced pressure to give a residue. The residue was purified by silica gel column chromatography (eluted with petroleum ether/ethyl acetate=30/1 to 3/1) to afford tert-butyl 4-(6-hydroxy-5-nitro-indazol-2-yl)piperidine-1-carboxylate (7.6 g, 20.97 mmol, 87% yield) as a yellow solid. ¹H NMR (400 MHz, CDCl₃) δ=10.00 (s, 1H), 8.75 (s, 1H), 8.18 (s, 1H), 7.24 (s, 1H), 4.56 (tt, J=4.0, 8.0 Hz, 1H), 4.42-4.29 (m, 2H), 2.95 (br t, J=21.2 Hz, 2H), 2.31-2.24 (m, 2H), 2.15-2.03 (m, 2H), 1.50 (s, 9H).

### Step 3:

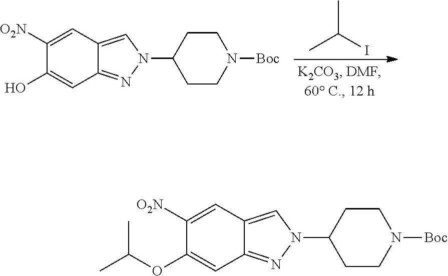

To a solution of tert-butyl 4-(6-hydroxy-5-nitro-indazol-2-yl)piperidine-1-carboxylate (7.6 g, 20.97 mmol, 1 eq) and 2-iodopropane (71.29 g, 419.40 mmol, 41.94 mL, 20 eq) in N,N-dimethylformamide (80 mL) was added potassium carbonate (8.69 g, 62.91 mmol, 3 eq). The mixture was stirred at 60° C. for 12 h. The reaction mixture was filtered and concentrated under reduced pressure to afford tert-butyl 4-(6-isopropoxy-5-nitro-indazol-2-yl) piperidine-1-carboxylate (6 g, 14.83 mmol, 71% yield) as a yellow solid which was used into the next step without further purification. MS (ESI) m/z: 405.21 [M+H]⁺. ¹H NMR (400 MHz, CDCl₃) δ=8.13 (s, 1H), 8.05 (s, 1H), 7.11 (s, 1H), 4.66 (td, J=6.0, 12.2 Hz, 1H), 4.52 (tt, J=4.0, 11.6 Hz, 1H), 4.33 (br d, J=2.8 Hz, 2H), 3.05-2.80 (m, 2H), 2.29-2.20 (m, 2H), 2.15-2.05 (m, 2H), 1.49 (s, 9H), 1.44-1.40 (m, 6H).

### Step 4:

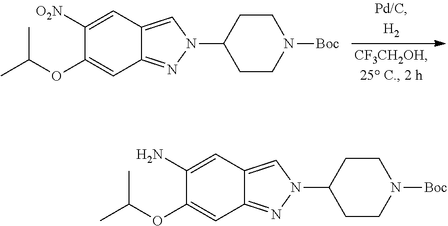 [SMILES: CC(C)Oc1cc2nn(C3CCN(C(=O)OC(C)(C)C)CC3)cc2cc1[N+](=O)[O-] ; CC(C)Oc1cc2nn(C3CCN(C(=O)OC(C)(C)C)CC3)cc2cc1N]

To a solution of tert-butyl 4-(6-isopropoxy-5-nitro-indazol-2-yl)piperidine-1-carboxylate (6 g, 14.83 mmol, 1 eq) in 2,2,2-trifluoroethanol (80 mL) was added palladium on carbon (2 g, 1.88 mmol, 10% purity, 1.27e-1 eq) under an atmosphere of nitrogen. The suspension was degassed under vacuum and purged with an atmosphere of hydrogen over several cycles. The mixture was stirred under a balloon of hydrogen (15 psi) at 25° C. for 2 h. The reaction mixture was filtered and the filtrate was concentrated to afford tert-butyl 4-(5-amino-6-isopropoxy-2H-indazol-2-yl)piperidine-1-carboxylate (5.5 g, 14.7 mmol, 99% yield) as a yellow solid which was used in the next step without further purification. MS (ESI) m/z: 375.1 [M+H]⁺.

### Step 5:

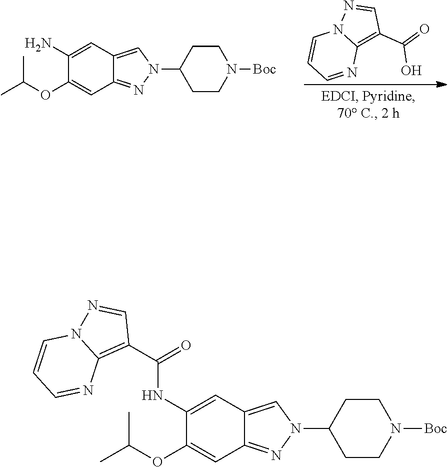 [SMILES: CC(C)Oc1cc2nn(C3CCN(C(=O)OC(C)(C)C)CC3)cc2cc1N ; O=C(O)c1cnn2cccnc12 ; CC(C)Oc1cc2nn(C3CCN(C(=O)OC(C)(C)C)CC3)cc2cc1NC(=O)c1cnn2cccnc12]

To a solution of tert-butyl 4-(5-amino-6-isopropoxy-2H-indazol-2-yl)piperidine-1-carboxylate (5.5 g, 14.69 mmol, 1 eq) and pyrazolo[1,5-a]pyrimidine-3-carboxylic acid (2.40 g, 14.7 mmol, 1 eq) in pyridine (60 mL) was added EDCI (8.53 g, 44.52 mmol, 3.03 eq). The mixture was stirred at 70° C. for 2 h. The reaction mixture was filtered and concentrated under reduced pressure to give a residue. The residue was purified by silica gel column chromatography (petroleum ether/ethyl acetate=100/1 to 50/1) to afford tert-butyl4-[6-isopropoxy-5-(pyrazolo[1,5-a]pyrimidine-3-carbonylamino)indazol-2-yl]piperidine-1-carboxylate (5.5 g, 10.6 mmol, 72% yield) as a yellow solid. MS (ESI) m/z: 520.2 [M+H]⁺.

### Step 6:

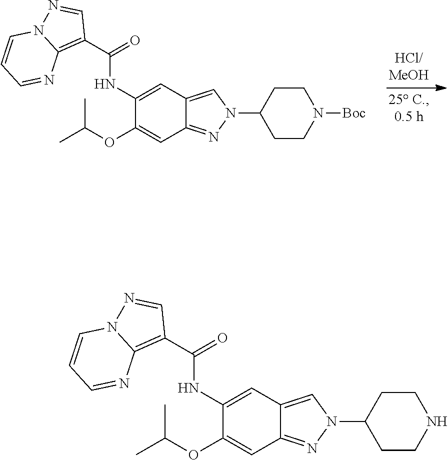 [SMILES: CC(C)Oc1cc2nn(C3CCN(C(=O)OC(C)(C)C)CC3)cc2cc1NC(=O)c1cnn2cccnc12 ; CC(C)Oc1cc2nn(C3CCNCC3)cc2cc1NC(=O)c1cnn2cccnc12]

To a solution of tert-butyl 4-(6-isopropoxy-5-(pyrazolo[1,5-a]pyrimidine-3-carboxamido)-2H-indazol-2-yl)piperidine-1-carboxylate (5.5 g, 10.6 mmol, 1 eq) in dichloromethane (30 mL) and methanol (30 mL) was added a solution of hydrogen chloride in methanol (4 M, 30 mL, 11.34 eq). The mixture was stirred at 25° C. for 0.5 hr. The reaction mixture was concentrated in vacuo to yield a residue which was suspended in a 10:1 mixture of dichloromethane:methanol (100 mL). The pH of the solution was then adjusted to pH=8 with the addition of ammonium hydroxide. The mixture was concentrated under reduced pressure to give a residue. The residue was purified by silica gel column chromatography (eluted with dichloromethane:methanol:ammonium hydroxide=50/1/0.1 to 10/1/0.1) to afford N-(6-isopropoxy-2-(piperidin-4-yl)-2H-indazol-5-yl)pyrazolo[1,5-a]pyrimidine-3-carboxamide (4.4 g, 10.49 mmol, 99% yield) as a white solid. ¹H NMR (400 MHz, DMSO-d₆) δ=10.60 (s, 1H), 9.38 (dd, J=1.2, 7.2 Hz, 1H), 9.16 (br s, 1H), 8.87 (dd, J=1.6, 4.0 Hz, 1H), 8.76 (s, 1H), 8.70 (s, 1H), 8.29 (s, 1H), 7.34 (dd, J=4.4, 7.2 Hz, 1H), 7.16 (s, 1H), 4.85 (td, J=6.0, 12.0 Hz, 1H), 4.74 (td, J=5.0, 10 Hz, 1H), 3.47-3.38 (m, 2H), 3.17-3.06 (m, 2H), 2.35-2.22 (m, 4H), 1.47 (d, J=6 Hz, 6H).

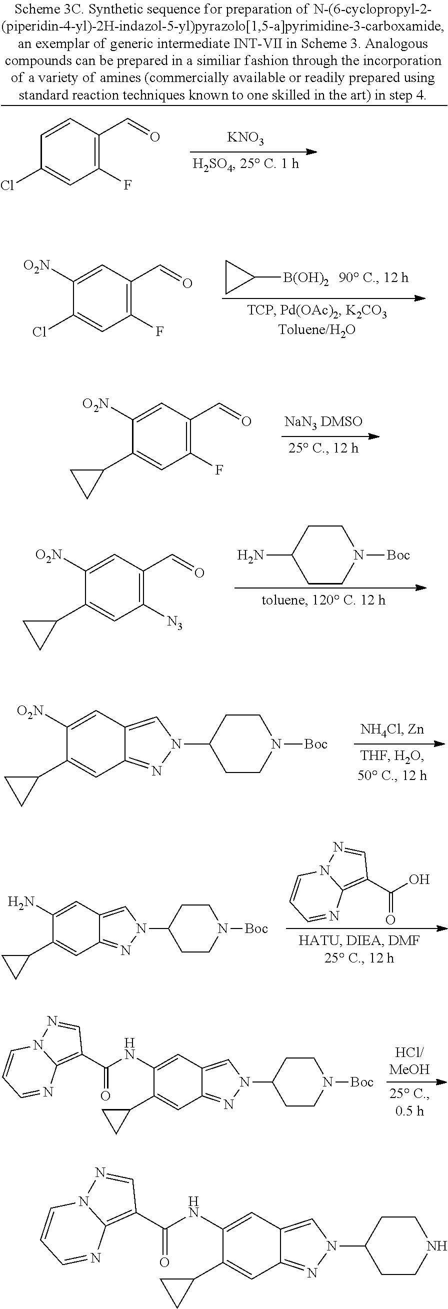

### Step 1:

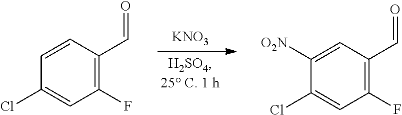

To a solution of 4-chloro-2-fluoro-benzaldehyde (5 g, 31.53 mmol, 1 eq) in concentrated sulfuric acid (50 mL) was added potassium nitrate (3.51 g, 34.7 mmol, 1.1 eq) at 0° C. The mixture was stirred at 25° C. for 0.5 h. The reaction mixture was quenched by the addition of water (100 mL) at 0° C., and then diluted with water (50 mL) and extracted with ethyl acetate (50 mL×3). The combined organic layers were washed with brine (50 mL×3), dried over anhydrous sodium sulfate, filtered, and concentrated under reduced pressure to afford 4-chloro-2-fluoro-5-nitro-benzaldehyde (6 g, 29.5 mmol, 93% yield) as a white solid which was used in subsequent reactions without purification. MS (ESI) m/z: 202.9 [M+H]⁺.

### Step 2:

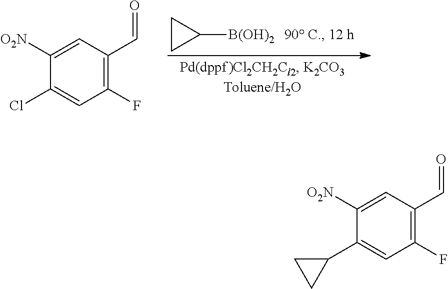

To a solution of 4-chloro-2-fluoro-5-nitro-benzaldehyde (1 g, 4.9 mmol, 1 eq) in dioxane (20 mL) and water (2 mL) was added cyclopropylboronic acid (844 mg, 9.83 mmol, 2 eq) and Pd(dppf)Cl₂.CH₂Cl₂ (401.2 mg, 491.3 μmol, 0.1 eq) and potassium carbonate (2.04 g, 14.74 mmol, 3 eq). The mixture was stirred at 90° C. for 12 h. The reaction mixture was quenched by the addition of water (100 mL) at 0° C., and then diluted with water (50 mL) and extracted with ethyl acetate (50 mL×3). The combined organic layers were washed with brine (50 mL×3), dried over anhydrous sodium sulfate, filtered, and concentrated under reduced pressure to give a residue. The residue was purified by column chromatography (silicon dioxide, eluted with petroleum ether/ethyl acetate=10/1 to 1/1) to afford 4-cyclopropyl-2-fluoro-5-nitro-benzaldehyde (0.55 g, 2.63 mmol, 54% yield) as a yellow oil. MS (ESI) m/z: 209.1 [M+H]⁺.

### Step 3:

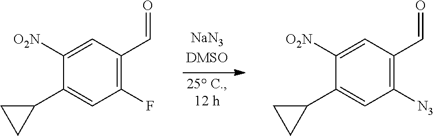 [SMILES: O=Cc1cc([N+](=O)[O-])c(C2CC2)cc1F ; [N-]=[N+]=Nc1cc(C2CC2)c([N+](=O)[O-])cc1C=O]

To a solution of 4-cyclopropyl-2-fluoro-5-nitro-benzaldehyde (0.55 g, 2.63 mmol, 1 eq) in dimethyl sulfoxide (5 mL) was added sodium azide (256.4 mg, 3.94 mmol, 1.5 eq). The mixture was stirred at 25° C. for 12 h. The reaction mixture was quenched by the addition of water (50 mL) at 25° C., and then diluted with water (50 mL) and extracted with ethyl acetate (50 mL×3). The combined organic layers were washed with brine (50 mL×3), dried over anhydrous sodium sulfate, filtered, and concentrated under reduced pressure to 2-azido-4-cyclopropyl-5-nitro-benzaldehyde (600 mg, 2.58 mmol, 98% yield), obtained as yellow oil and used in subsequent reactions without further purification. MS (ESI) m/z: 232.2 [M+H]⁺.

### Step 4:

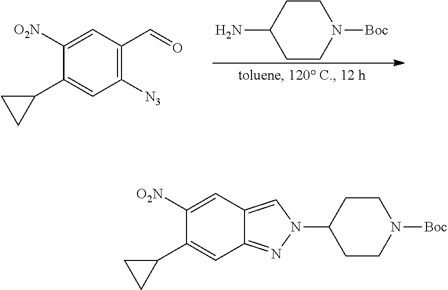 [SMILES: [N-]=[N+]=Nc1cc(C2CC2)c([N+](=O)[O-])cc1C=O ; CC(C)(C)OC(=O)N1CCC(N)CC1 ; CC(C)(C)OC(=O)N1CCC(n2cc3cc([N+](=O)[O-])c(C4CC4)cc3n2)CC1]

To a solution of 2-azido-4-cyclopropyl-5-nitro-benzaldehyde (0.6 g, 2.58 mmol, 1 eq) in toluene (6 mL) was added tert-butyl 4-aminopiperidine-1-carboxylate (569.28 mg, 2.84 mmol, 1.1 eq). The mixture was stirred at 120° C. for 12 h. The reaction mixture was concentrated under reduced pressure to give a residue. The residue was purified by column chromatography (silicon dioxide, eluted with petroleum ether/ethyl acetate=5/1 to 1/1) to afford tert-butyl 4-(6-cyclopropyl-5-nitro-indazol-2-yl)piperidine-1-carboxylate (0.7 g, 1.81 mmol, 70% yield) as a yellow solid. MS (ESI) m/z: 386.2 [M+H]⁺.

### Step 5:

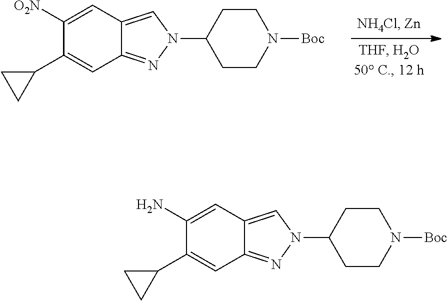 [SMILES: CC(C)(C)OC(=O)N1CCC(n2cc3cc([N+](=O)[O-])c(C4CC4)cc3n2)CC1 ; CC(C)(C)OC(=O)N1CCC(n2cc3cc(N)c(C4CC4)cc3n2)CC1]

To a solution of tert-butyl 4-(6-cyclopropyl-5-nitro-indazol-2-yl) piperidine-1-carboxylate (0.7 g, 1.81 mmol, 1 eq) in tetrahydrofuran (7 mL) was added zinc powder (710.7 mg, 10.9 mmol, 6 eq) and ammonium chloride (968.9 mg, 18.11 mmol, 10 eq). The mixture was stirred at 50° C. for 12 h. The reaction mixture was filtered and concentrated under reduced pressure to give a residue. The residue was purified by column chromatography (silicon dioxide, eluted with petroleum ether/ethyl acetate=5/1 to 1/1) to afford tert-butyl 4-(5-amino-6-cyclopropyl-indazol-2-yl) piperidine-1-carboxylate (0.6 g, 1.68 mmol, 93% yield) as a yellow oil. MS (ESI) m/z: 356.2 [M+H]⁺.

### Step 6:

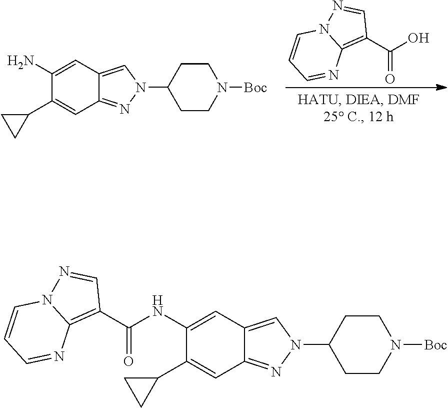 [SMILES: CC(C)(C)OC(=O)N1CCC(n2cc3cc(N)c(C4CC4)cc3n2)CC1 ; O=C(O)c1cnn2cccnc12 ; CC(C)(C)OC(=O)N1CCC(n2cc3cc(NC(=O)c4cnn5cccnc45)c(C4CC4)cc3n2)CC1]

To a solution of tert-butyl 4-(5-amino-6-cyclopropyl-indazol-2-yl)piperidine-1-carboxylate (0.5 g, 1.40 mmol, 1 eq) in N,N-dimethylformamide (50 mL) was added pyrazolo[1,5-a]pyrimidine-3-carboxylic acid (228.82 mg, 1.40 mmol, 1 eq) and (dimethylamino(triazolo(4,5-b)pyridin-3-yloxy)methylene)-dimethy)-ammonium;hexafluorophosphate (640 mg, 1.68 mmol, 1.2 eq) and diisopropylethylamine (543.86 mg, 4.21 mmol, 732.96 μL, 3 eq). The mixture was stirred at 25° C. for 12 h. The reaction mixture was concentrated under reduced pressure to give a residue. The residue was purified by column chromatography (silicon dioxide, eluted with petroleum ether/ethyl acetate=3/1 to 1/1) to afford tert-butyl4-[6-cyclopropyl-5-(pyrazolo[1,5-a]pyrimidine-3-carbonylamino)indazol-2-yl]piperidine-1-carboxylate (0.45 g, 897.2 μmol, 64% yield) as a yellow solid. MS (ESI) m/z: 501.2 [M+H]⁺.

### Step 7:

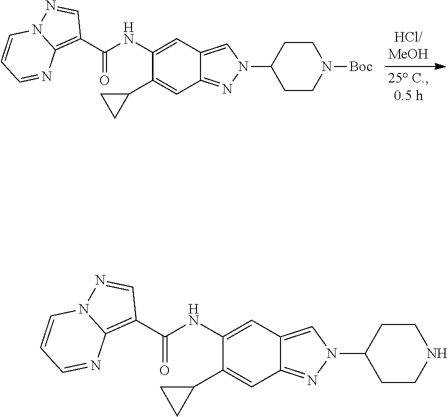 [SMILES: CC(C)(C)OC(=O)N1CCC(n2cc3cc(NC(=O)c4cnn5cccnc45)c(C4CC4)cc3n2)CC1 ; O=C(Nc1cc2cn(C3CCNCC3)nc2cc1C1CC1)c1cnn2cccnc12]

To a solution of tert-butyl 4-[6-cyclopropyl-5-(pyrazolo[1,5-a]pyrimidine-3-carbonylamino)indazol-2-yl]piperidine-1-carboxylate (0.45 g, 897.2 μmol, 1 eq) in dichloromethane (10 mL) was added a solution of hydrogen chloride in methanol (4 M, 10 mL, 44.58 eq). The mixture was stirred at 25° C. for 0.5 h. The reaction mixture was concentrated under reduced pressure to give a residue, the pH of the solution was adjusted to 7 by addition of an aqueous solution of sodium bicarbonate (20 mL) in tetrahydrofuran (100 mL). The mixture was filtered and concentrated to afford N-[6-cyclopropyl-2-(4-piperidyl)indazol-5-yl]pyrazolo[1,5-a]pyrimidine-3-carboxamide (350 mg, 871.81 μmol, 97% yield) as a white solid which was used in the next step without further purification. MS (ESI) m/z: 401.2 [M+H]⁺.

TABLE 3
The following compounds may be prepared in an analogous fashion as described for the
synthesis of Example 118.

| | | ¹H NMR |
|---|---|---|
| | | All spectra acquired at 400 MHz |
| Example | MS | unless otherwise indicated |
| Example 118 | See Above | |
| 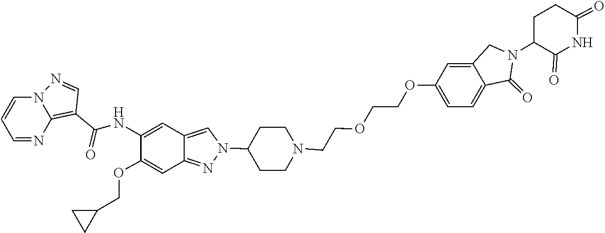 [SMILES: O=C1CCC(N2Cc3cc(OCCOCCN4CCC(n5cc6cc(NC(=O)c7cnn8cccnc78)c(OCC7CC7)cc6n5)CC4)ccc3C2=O)C(=O)N1] | 762.3 | CDCl₃ δ = 10.57 (s, 1H), 8.94 (s, 1H), 8.85-8.80 (m, 2H), 8.68 (d, J = 3.2 Hz, 1H), 8.55 (br s, 1H), 7.93 (br d, J = 8.4 Hz, 1H), 7.85-7.78 (m, 2H), 7.44-7.39 (m, 1H), 7.07- 6.97 (m, 4H), 5.20 (dd, J = 5.6, 13.3 Hz, 1H), 4.46-4.36 (m, 2H), 4.30-4.20 (m, 1H), 4.30-4.20 (m, 3H), 4.01 (d, J = 6.4 Hz, 2H), 3.90- 3.81 (m, 4H), 3.81-3.81 (m, 1H), 3.31 (br d, J = 11.6 Hz, 2H), 2.95- 2.77 (m, 4H), 2.52 (br s, 1H), 2.35- 2.27 (m, 1H), 2.31 (dt, J = 4.8, 13.2 Hz, 6H), 2.19 (br d, J = 8.8 Hz, 2H), 1.50 (br s, 1H), 0.77-0.68 (m, 2H), |
| Example 119 | | 0.56-0.49 (m, 2H). |
| 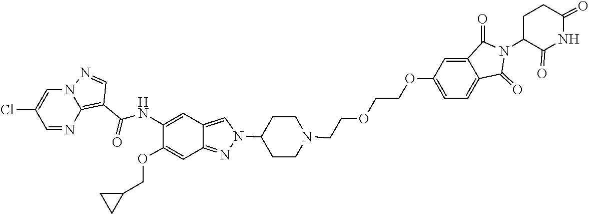 [SMILES: O=C1CCC(N2C(=O)c3ccc(OCCOCCN4CCC(n5cc6cc(NC(=O)c7cnn8cc(Cl)cnc78)c(OCC7CC7)cc6n5)CC4)cc3C2=O)C(=O)N1] | 810.3 | DMSO-d₆ δ = 11.07-11.15 (m, 1 H) 10.35-10.47 (m, 1 H) 9.78-9.89 (m, 1 H) 8.84-8.92 (m, 1 H) 8.68-8.77 (m, 2 H) 8.19-8.33 (m, 2 H) 7.79-7.89 (m, 1 H) 7.43-7.51 (m, 1 H) 7.33-7.40 (m, 1 H) 7.01-7.10 (m, 1 H) 5.08-5.15 (m, 1 H) 4.29-4.38 (m, 3 H) 3.98- 4.05 (m, 2 H) 3.79 (br dd, J = 5.07, 3.56 Hz, 2 H) 3.62 (t, J = 5.82 Hz, 2 H) 3.01 (br d, J = 11.26 Hz, 2 H) 2.83-2.93 (m, 1 H) 2.53-2.58 (m, 3 H) 2.14-2.22 (m, 2 H) 1.98-2.09 (m, 5 H) 1.46-1.56 (m, 1 H) 0.65-0.73 (m, 2 H) 0.40-0.46 (m, 2H) |
| Example 120 | | |
| 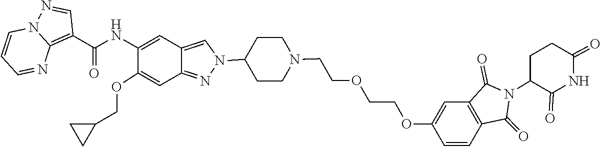 [SMILES: O=C1CCC(N2C(=O)c3ccc(OCCOCCN4CCC(n5cc6cc(NC(=O)c7cnn8cccnc78)c(OCC7CC7)cc6n5)CC4)cc3C2=O)C(=O)N1] | 776.3 | CDCl₃ δ = 10.56 (s, 1H), 8.92 (s, 1H), 8.85-8.80 (m, 2H), 8.76-8.65 (m, 2H), 8.35 (s, 1H), 7.86 (s, 1H), 7.80 (d, J = 8.4 Hz, 1H), 7.42 (d, J = 2.0 Hz, 1H), 7.24 (dd, J = 2.4, 8.4 Hz, 1H), 7.06- 7.00 (m, 2H), 4.96 (dd, J = 5.2, 12.4 Hz, 1H), 4.51-4.42 (m, 1H), 4.37- 4.24 (m, 2H), 4.03 (d, J = 6.8 Hz, 2H), 3.92-3.74 (m, 4H), 3.29 (br t, J = 10.4 Hz, 2H), 2.94-2.77 (m, 4H), |
| Example 121 | | 2.52-2.44 (m, 4H), 2.18-2.12 (m, 1H), |
| | | 1.55-1.46 (m, 1H), 0.76-0.69 (m, 2H), |
| | | 0.54 (q, J = 4.8 Hz, 2H) |
| 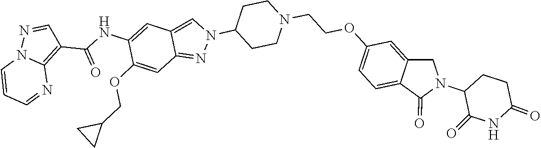 [SMILES: O=C1CCC(N2Cc3cc(OCCN4CCC(n5cc6cc(NC(=O)c7cnn8cccnc78)c(OCC7CC7)cc6n5)CC4)ccc3C2=O)C(=O)N1] | 718.2 | DMSO-d₆ δ = 10.97 (s, 1 H), 10.64 (s, 1H), 9.37 (dd, J = 7.2, 1.6 Hz, 1 H), 8.86 (dd, J = 4.0, 1.6 Hz, 1 H), 8.74 (s, 1 H), 8.71 (s, 1 H), 8.30 (s, 1 H), 7.63 (d, J = 8.4 Hz, 1 H), 7.28-7.33 (m, 1H), 7.21 (d, J = 1.6 Hz, 1 H), 7.08 (dd, J = 8.4, 2.0 Hz, 1 H), 7.03 (s, 1 H), 5.08 (dd, J = 13.2, 5.2 Hz, 1 H), 4.34-4.42 (m, 2 H), 4.24-4.30 (m, 1 H), 4.22 (t, J = 5.6 Hz, 2 H), 4.01 (d, J = 6.8 Hz, 2 H), 3.09 (d, J = 11.2 Hz, 2 H), 2.86-2.96 (m, 1 H), 2.81 (t, J = 5.6 |
| Example 122 | | Hz, 2 H), 2.59 (d, J = 16.0 Hz, 1 H), |
| | | 2.25-2.42 (m, 3 H), 2.09 (d, J = 3.2 |
| | | Hz, 4H), 1.99 (dd, J = 10.4, 5.2 Hz, 1 |
| | | H), 1.43-1.54 (m, 1 H), 0.67-0.74 |
| | | (m, 2 H), 0.45 (q, J = 4.8 Hz, 2H). |
| 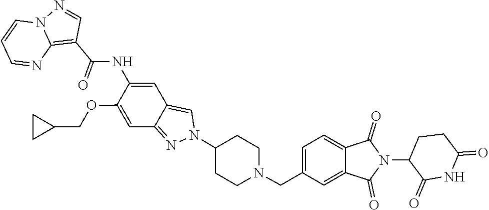 [SMILES: O=C1CCC(N2C(=O)c3ccc(CN4CCC(n5cc6cc(NC(=O)c7cnn8cccnc78)c(OCC7CC7)cc6n5)CC4)cc3C2=O)C(=O)N1] | 702.4 | CDCl₃ δ = 10.57 (s, 1 H), 8.91 (s, 1 H), 8.86-8.79 (m, 2 H), 8.69 (dd, J = 1.6, 4.4 Hz, 1 H), 8.01-7.93 (m, 2 H), 7.90-7.84 (m, 2 H), 7.82- 7.77 (d, J = 8.0 Hz, 1 H), 7.07- 7.01 (m, 2 H), 5.00 (dd, J = 5.2, 12.0 Hz, 1 H), 4.43-4.33 (m, 1 H), 4.03 (d, J = 6.8 Hz, 2 H), 3.72 (s, 2 H), 3.07-2.77 (m, 5 H), 2.34-2.20 (m, 7 H), 1.53 (m, 1 H), 0.73 (d, J = 8.0 Hz, 2 H), 0.54 (d, J = 5.2 Hz, 2H) |
| Example 123 | | |
| 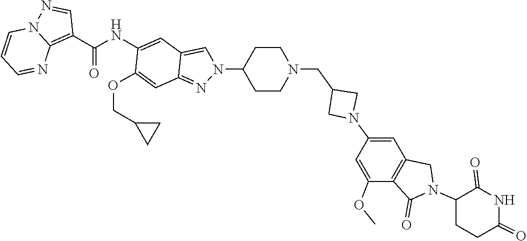 [SMILES: COc1cc(N2CC(CN3CCC(n4cc5cc(NC(=O)c6cnn7cccnc67)c(OCC6CC6)cc5n4)CC3)C2)cc2c1C(=O)N(C1CCC(=O)NC1=O)C2] | 773.3 | DMSO-d₆ δ = 10.89 (s, 1H), 10.65 (s, 1H), 9.43-9.31 (m, 1H), 8.89-8.81 (m, 1H), 8.73 (d, J = 13.2 Hz, 2H), 8.30 (s, 1H), 8.23 (s, 1H), 7.31 (dd, J = 6.8, 4.4 Hz, 1H), 7.03 (s, 1H), 6.05 (s, 1H), 5.91 (s, 1H), 4.94 (dd, J = 13.2, 5.2 Hz, 1H), 4.42-4.29 (m, 1H), 4.24-4.14 (m, 1H), 4.09 (s, 1H), 4.06-3.99 (m, 4H), 3.80 (s, 3H), 3.59 (t, J = 5.2 Hz, 2H), 3.00-2.95 (m, 2H), 2.95-2.81 (m, 2H), 2.68-2.63 (m, 2H), 2.53-2.60 (m, 1H), 2.27-2.34 (m, 1H), 2.12-2.23 (m, 2H), 2.01-2.11 (m, 4H), 1.86-1.94 (m, 1H), 1.44-1.54 (m, 1H), 0.67-0.74 (m, 2H), 0.42-0.48 |
| Example 124 | | (m, 2H) |
| 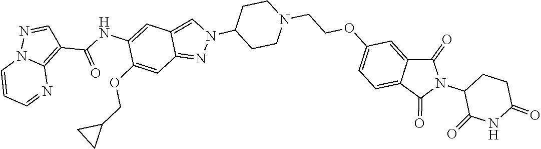 [SMILES: O=C1CCC(N2C(=O)c3ccc(OCCN4CCC(n5cc6cc(NC(=O)c7cnn8cccnc78)c(OCC7CC7)cc6n5)CC4)cc3C2=O)C(=O)N1] | 732.2 | DMSO-d₆ δ = 11.11 (br s, 1H), 10.64 (s, 1H), 9.37 (d, J = 6.8 Hz, 1H), 8.87 (d, J = 2.4 Hz, 1H), 8.72 (d, J = 10.0 Hz, 2H), 8.30 (s, 1H), 7.84 (d, J = 8 Hz 1H), 7.49 (s, 1H), 7.39 (dd, J = 2.0, 8.4 Hz, 1H), 7.32 (dd, J = 4.4, 6.8 Hz, 1H), 7.04 (s, 1H), 5.12 (dd, J = 5.6, 13.4 Hz, 1H), 4.39-4.33 (m, 3H), 4.02 (d, J = 6.8 Hz, 2H), 3.10 (d, J = 10.8 Hz, 2H), |
| Example 125 | | 2.93-2.77 (m, 5H), 2.36-2.09 (m, |
| | | 7H), 1.49-1.48 (m, 1H), 0.70-0.69 |
| | | (m, 2H), 0.45-0.44 (m, 2H) |
| 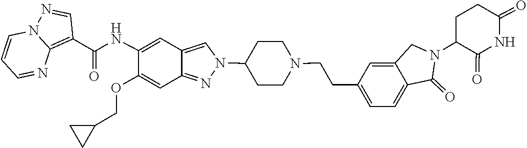 [SMILES: O=C1CCC(N2Cc3cc(CCN4CCC(n5cc6cc(NC(=O)c7cnn8cccnc78)c(OCC7CC7)cc6n5)CC4)ccc3C2=O)C(=O)N1] | 702.2 | DMSO-d₆ δ = 10.98 (s, 1 H) 10.65 (s, 1 H) 9.37 (dd, J = 6.8, 1.6 Hz, 1 H) 8.87 (dd, J = 4.0, 1.6 Hz, 1 H) 8.72 (d, J = 10.0 Hz, 2 H) 8.31 (s, 1 H) 8.27 (s, 1 H) 7.65 (d, J = 8.0 Hz, 1 H) 7.50 (s, 1 H) 7.41 (d, J = 8.0 Hz, 1 H) 7.30-7.33 (m, 1 H) 7.04 (s, 1 H) 5.11 (dd, J = 13.2, 5.2 Hz, 1 H) 4.26-4.48 (m, 3 H) 4.02 (d, J = 6.8 |
| Example 126 | | Hz, 2 H) 3.08-3.07 (m, 2 H) 2.86-2.94 |
| | | (m, 3 H) 2.40-2.39 (m, 1 H) 0.68-0.73 |
| | | (m, 2 H) 0.43-0.48 (m, 2 H) |
| 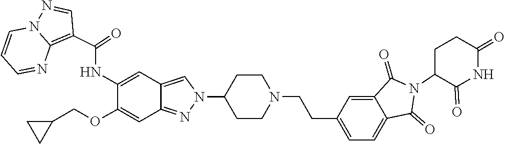 [SMILES: O=C1CCC(N2C(=O)c3ccc(CCN4CCC(n5cc6cc(NC(=O)c7cnn8cccnc78)c(OCC7CC7)cc6n5)CC4)cc3C2=O)C(=O)N1] | 716.2 | DMSO-d₆ δ = 11.12 (s, 1 H), 10.64 (s, 1H), 9.37 (dd, J = 7.2, 1.6 Hz, 1 H), 8.87 (dd, J = 4.4, 1.6 Hz, 1 H), 8.72 (d, J = 9.2 Hz, 2 H), 8.30 (s, 1 H), 7.85 (d, J = 4.8 Hz, 2 H), 7.74-7.81 (m, 1 H), 7.28-7.35 (m, 1 H), 7.04 (s, 1 H), 5.14 (dd, J = 12.8, 5.4 Hz, 1 H), 4.37 (dt, J = 10.4, 5.2 Hz, 1 H), 4.02 (d, J = 6.8 Hz, 2 H), 3.08 (d, J = 11.2 Hz, 2 |
| Example 127 | | H), 2.99 (t, J = 7.2 Hz, 2 H), 2.84-2.92 |
| | | (m, 1 H), 2.59-2.70 (m, 4 H), 2.15-2.25 |
| | | (m, 2H),2.07 (d, J = 4.4 Hz, 5 H), 1.44- |
| | | 1.57 (m, 1 H), 0.67-0.74 (m, 2 H), 0.45 |
| | | (q, J = 4.8 Hz, 2H). |
| 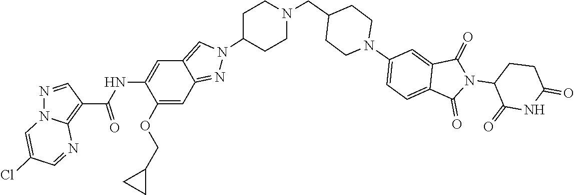 [SMILES: O=C1CCC(N2C(=O)c3ccc(N4CCC(CN5CCC(n6cc7cc(NC(=O)c8cnn9cc(Cl)cnc89)c(OCC8CC8)cc7n6)CC5)CC4)cc3C2=O)C(=O)N1] | 819.3 | DMSO-d₆ δ = 11.08 (br s, 1 H), 10.43 (s, 1 H), 9.83 (d, J = 2.4 Hz, 1 H), 8.89 (d, J = 2.0 Hz, 1 H), 8.73 (d, J = 6.0 Hz, 2 H), 8.35 (s, 1 H), 8.31 (s, 1 H), 7.65 (d, J = 8.4 Hz, 1 H), 7.32 (d, J = 2.0 Hz, 1 H), 7.24 (dd, J = 2.2, 8.8 Hz, 1 H), 7.04 (s, 1 H), 5.07 (dd, J = 5.6, 12.8 Hz, 1 H), 4.36 (br s, 1 H), 4.10- 4.01 (m, 4 H), 3.04-2.84 (m, 6 H), 2.62-2.53 (m, 3 H), 2.21 (br d, J = 6.8 Hz, 2 H), 2.14-2.00 (m, 7 H), 1.89-1.75 (m, 3 H), 1.58-1.42 (m, 1H), 1.17 (br d, |
| Example 128 | | J = 12.8 Hz, 2 H), 0.74-0.64 (m, 2 H), |
| | | 0.48-0.38 (m, 2H) |
| 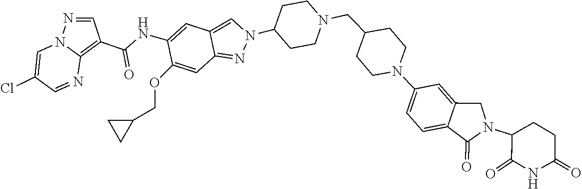 [SMILES: O=C1CCC(N2Cc3cc(N4CCC(CN5CCC(n6cc7cc(NC(=O)c8cnn9cc(Cl)cnc89)c(OCC8CC8)cc7n6)CC5)CC4)ccc3C2=O)C(=O)N1] | 805.3 | DMSO-d₆ δ = 10.94 (s, 1 H), 10.44 (s, 1H), 9.83 (d, J = 2.2 Hz, 1 H), 9.38-9.09 (m, 1 H), 8.88 (d, J = 2.2 Hz, 1 H), 8.81-8.70 (m, 2 H), 8.32 (s, 1 H), 7.53 (d, J = 8.8 Hz, 1 H), 7.15-6.98 (m, 3 H), 5.04 (dd, J = 5.2, 13.2 Hz, 1 H), 4.93-4.62 (m, 1 H), 4.38-4.28 (m, 1 H), 4.26- 4.14 (m, 1 H), 4.03 (d, J = 7.2 Hz, 2 H), 3.93 (br d, J = 12.4 Hz, 2 H), 3.72 (br d, J = 11.2 Hz, 2 H), 3.18 (br d, J = 12.4 Hz, 2 H), 3.08 (br s, |
| Example 129 | | 2 H), 2.96-2.81 (m, 3 H), 2.63- |
| | | 2.53 (m, 2 H), 2.46-2.34 (m, 4 H), |
| | | 2.09 (br d, J = 18.4 Hz, 1 H), 2.01- |
| | | 1.78 (m, 3 H), 1.61-1.45 (m, 1 |
| | | H), 1.32 (br d, J = 12.0 Hz, 2 H), |
| | | 0.78-0.63 (m, 2 H), 0.50-0.34 (m, 2H) |
| 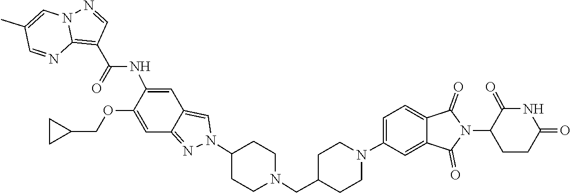 [SMILES: Cc1cnc2c(C(=O)Nc3cc4cn(C5CCN(CC6CCN(c7ccc8c(c7)C(=O)N(C7CCC(=O)NC7=O)C8=O)CC6)CC5)nc4cc3OCC3CC3)cnn2c1] | 799.3 | CDCl₃ δ : 10.40-10.60 (m, 1 H) 8.89- 8.95 (m, 1 H) 8.69-8.76 (m, 1 H) 8.60-8.66 (m, 1 H) 8.50- 8.57 (m, 1 H) 7.97-8.08 (m, 1 H) 7.83-7.92 (m, 1 H) 7.67-7.74 (m, 1 H) 7.28-7.31 (m, 1 H) 7.03- 7.11 (m, 1 H) 6.96-7.03 (m, 1 H) 6.95-7.02 (m, 1 H) 4.85-5.08 (m, 1 H) 4.32-4.52 (m, 1 H) 3.89- 4.10 (m, 4 H) 3.07-3.30 (m, 2 H) 2.96-3.05 (m, 2 H) 2.71-2.96 (m, 3 H) 2.68-2.94 (m, 3 H) 2.46- |
| Example 130 | | 2.54 (m, 3 H) 2.20-2.44 (m, 6 H) |
| | | 2.08-2.18 (m, 1 H) 1.83-2.03 (m, |
| | | 3 H) 1.47-1.55 (m, 2 H) 1.19- |
| | | 1.41 (m, 3 H) 0.67-0.76 (m, 2 H) |
| | | 0.48-0.59 (m, 2 H) |
| 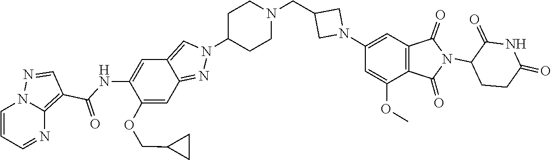 [SMILES: COc1cc(N2CC(CN3CCC(n4cc5cc(NC(=O)c6cnn7cccnc67)c(OCC6CC6)cc5n4)CC3)C2)cc2c1C(=O)N(C1CCC(=O)NC1=O)C2=O] | 787.3 | DMSO-d₆ δ = 11.04 (s, 1 H) 10.65 (s, 1 H) 9.35 (dd, J = 7.2 Hz 1.6 Hz, 1 H) 8.85 (dd, J = 4.4 Hz 1.6 Hz, 1 H) 8.84 (s, 1 H) 8.74 (s, 1 H) 8.70 (s, 1 H) 8.30 (s, 1 H) 7.30 (dd, J = 6.8 Hz 4.4 Hz, 1 H) 7.03 (s, 1 H) 6.38 (s, 1 H) 6.10 (s, 1 H) 5.01- 4.93 (m, 1 H) 4.41-4.30 (m, 1 H) 4.15 (t, J = 8.4 Hz, 2 H) 4.02 (d, J = 6.8 Hz, 2 H) 3.88 (s, 3 H) 3.75- |
| Example 131 | | 3.69 (m, 2 H) 3.02-2.93 (m, 3 H) |
| | | 2.88-2.80 (m, 1 H) 2.70-2.60 (m, |
| | | 3 H) 2.22-2.14 (m, 2 H) 2.10- |
| | | 2.02 (m, 4 H) 1.99-1.94 (m, 1 H) |
| | | 1.53-1.45 (m, 1H) 1.26-1.19 (m, 1 H) |
| | | 0.75-0.67 (m, 2 H) 0.49-0.41 (m, 2H) |
| 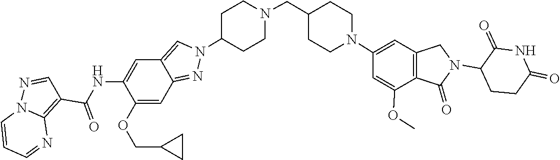 [SMILES: COc1cc(N2CCC(CN3CCC(n4cc5cc(NC(=O)c6cnn7cccnc67)c(OCC6CC6)cc5n4)CC3)CC2)cc2c1C(=O)N(C1CCC(=O)NC1=O)C2] | 801.4 | DMSO-d₆ δ = 10.89 (s, 1 H) 10.65 (s, 1 H) 9.36 (dd, J = 6.8 Hz 1.2 Hz, 1 H) 8.87 (dd, J = 4.0 Hz 1.6 Hz, 1 H) 8.74 (s, 1 H) 8.71 (s, 1 H) 8.30 (s, 1 H) 7.89 (s, 1 H) 7.31 (dd, J = 6.8 Hz 4.0 Hz, 1 H) 7.03 (s, 1 H) 6.60 (s, 1 H) 6.46 (s, 1 H) 5.00- 4.90 (m, 1 H) 4.41-4.30 (m, 1 H) 4.26-4.18 (m, 1 H) 4.14-4.16 (m, 1 H) 4.02 (dd, J = 6.8 Hz, 2 H) 3.94 |
| Example 132 | | 3.96 (m, 2 H) 3.83 (s, 3 H) 3.03- |
| | | 2.94 (m, 2 H) 2.91-2.80 (m, 3 H) |
| | | 2.35-2.28 (m, 1 H) 2.25-2.18 (m, |
| | | 2 H) 2.15-2.03 (m, 6 H) 1.95-1.87 |
| | | (m, 1 H) 1.85-1.72 (m, 3 H) 1.55-1.44 |
| | | (m, 1 H) 1.30-1.09 (m, 3 H) 0.77-0.65 |
| | | (m, 2 H) 0.52-0.38 (m, 2 H) |
| 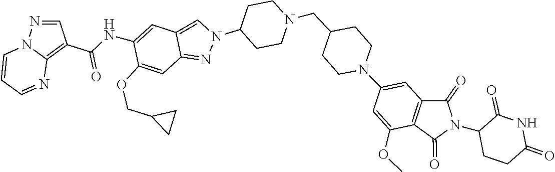 [SMILES: COc1cc(N2CCC(CN3CCC(n4cc5cc(NC(=O)c6cnn7cccnc67)c(OCC6CC6)cc5n4)CC3)CC2)cc2c1C(=O)N(C1CCC(=O)NC1=O)C2=O] | 815.3 | DMSO-d₆ δ = 11.05 (s, 1 H) 10.65 (s, 1 H) 9.37 (dd, J = 6.8 Hz 1.2 Hz, 1 H) 8.86 (d, J = 2.8 Hz, 1 H) 8.74 (s, 1 H) 8.71 (s, 1 H) 8.30 (s, 1 H) 7.31 (dd, J = 6.8 Hz 4.0 Hz, 1 H) 7.03 (s, 1 H) 6.93 (s, 1 H) 6.63 (s, 1 H) 5.05-4.95 (m, 1 H) 4.40- 4.30 (m, 1 H) 4.12-4.04 (m, 2 H) 4.04-3.99 (m, 2 H) 3.92 (s, 3 H) 3.04-2.94 (m, 4 H) 2.89-2.81 (m, 1 H) 2.62-2.55 (m, 1 H) 2.24- |
| Example 133 | | 2.17 (m, 2 H) 2.13-2.07 (m, 6 H) |
| | | 1.99-1.94 (m, 1 H) 1.90-1.78 (m, |
| | | 3 H) 1.55-1.45 (m, 1 H) 1.29- |
| | | 1.09 (m, 3 H) 0.76-0.65 (m, 2 H) |
| | | 0.51-0.40 (m, 2H) |
| 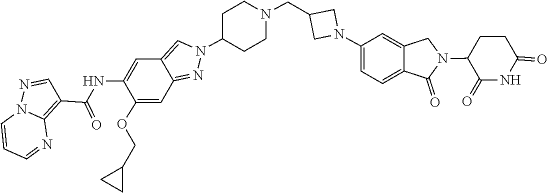 [SMILES: O=C1CCC(N2Cc3cc(N4CC(CN5CCC(n6cc7cc(NC(=O)c8cnn9cccnc89)c(OCC8CC8)cc7n6)CC5)C4)ccc3C2=O)C(=O)N1] | 743.3 | CDCl₃ δ = 10.57 (s, 1H), 8.91 (s, 1H), 8.85-8.80 (m, 2H), 8.70 (d, J = 2.4 Hz, 1H), 8.07-8.02 (m, 1H), 7.92-7.86 (m, 2H), 7.71 (d, J = 8.4 Hz, 1H), 7.08-6.99 (m, 2H), 6.47 (d, J = 8.8 Hz, 2H), 6.41-6.35 (m, 1H), 6.39 (s, 1H), 5.20 (br d, J = 12.4 Hz, 1H), 4.46-4.35 (m, 2H), 4.30-4.21 (m, 1H), 4.31- 4.20 (m, 1H), 4.10 (br d, J = 8.8 Hz, 2H), 4.03 (d, J = 6.8 Hz, 2H), 3.67 (s, 2H), 3.07 (s, 3H), 2.96-2.81 |
| Example 134 | | (m, 2H), 2.77 (s, 2H), 2.27 (br s, |
| | | 8H), 0.73 (br d, J = 7.6 Hz, 2H), |
| | | 0.54 (d, J = 5.6 Hz, 2H). |
| 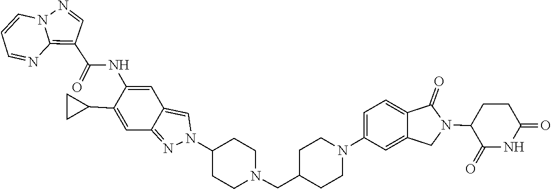 [SMILES: O=C1CCC(N2Cc3cc(N4CCC(CN5CCC(n6cc7cc(NC(=O)c8cnn9cccnc89)c(C8CC8)cc7n6)CC5)CC4)ccc3C2=O)C(=O)N1] | 741.3 | DMSO-d₆ δ = 12.95 (s, 1H), 10.95 (s, 1H), 8.74-8.46 (m, 2H), 8.16 (s, 1H), 8.09 (s, 1H), 7.95 (s, 1H), 7.75- 7.56 (m, 3H), 7.52 (d, J = 8.4 Hz, 1H), 7.27 (t, J = 8.4 Hz, 1H), 7.15-6.96 (m, 4H), 5.44-5.18 (m, 1H), 5.05 (d d, J = 4.8, 13.2 Hz, 1H), 4.63 (d, J = 1.2 Hz, 1H), 4.46-4.12 (m, 3H), 3.48 (s, 2H), 3.40 (s, 2H), 2.89 (s, 3H), 2.74-2.65 (m, 6H), 2.41-2.28 (m, 2H), 2.20-1.91 (m, 6H), 1.75-1.55 (m, 2H), 1.54-1.35 (m, 3H) |
| Example 135 | | |
| 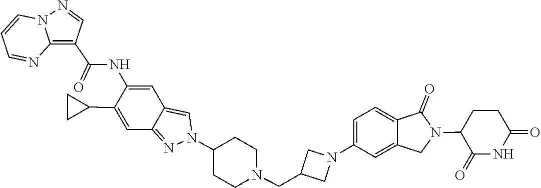 [SMILES: O=C1CCC(N2Cc3cc(N4CC(CN5CCC(n6cc7cc(NC(=O)c8cnn9cccnc89)c(C8CC8)cc7n6)CC5)C4)ccc3C2=O)C(=O)N1] | 713.3 | DMSO-d₆ δ = 10.93 (s, 1 H), 10.35 (s, 1H), 9.35 (dd, J = 7.2, 1.6 Hz, 1 H), 8.90 (dd, J = 4.0, 1.6 Hz, 1 H), 8.72 (s, 1 H), 8.63 (s, 1 H), 8.36 (s, 1 H), 8.14 (s, 1 H), 7.48 (d, J = 8.4 Hz, 1 H), 7.41 (s, 1 H), 7.27- 7.34 (m, 1 H), 6.44-6.55 (m, 2 H), 5.01 (dd, J = 13.2, 5.2 Hz, 1 H), 4.42 (dt, J = 15.2, 7.6 Hz, 1 H), 4.26-4.34 (m, 1 H), 4.13-4.21 (m, 1 H), 4.03 (t, J = 7.2 Hz, 2 H), 2.87- 3.02 (m, 4 H), 2.54-2.70 (m, 4 H), |
| Example 136 | | 2.31-2.38 (m, 1 H), 2.16-2.25 (m, 2 |
| | | H), 2.04-2.12 (m, 6 H), 1.91-2.00 |
| | | (m, 1 H), 1.13-1.21 (m, 2 H), 0.72- |
| | | 0.80 (m, 2 H). |
| 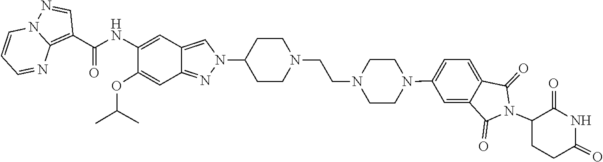 [SMILES: CC(C)Oc1cc2nn(C3CCN(CCN4CCN(c5ccc6c(c5)C(=O)N(C5CCC(=O)NC5=O)C6=O)CC4)CC3)cc2cc1NC(=O)c1cnn2cccnc12] | 788.3 | DMSO-d₆ δ = 11.08 (s, 1 H), 10.59 (s, 1H), 9.38 (dd, J = 1.6, 7.0 Hz, 1 H), 8.87 (dd, J = 1.2, 4.0 Hz, 1 H), 8.72 (d, J = 14.8 Hz, 2 H), 8.30 (s, 1 H), 7.68 (d, J = 8.4 Hz, 1 H), 7.37-7.30 (m, 2 H), 7.28-7.22 (m, 1 H), 7.13 (s, 1 H), 5.07 (dd, J = 5.2, 12.8 Hz, 1 H), 4.85 (m, 1 H), 4.46-4.28 (m, 1 H), 3.48-3.45 (m, 5 H), 3.04 (m, 2 H), 2.94-2.83 (m, 1 H), 2.66-2.52 (m, 9 H), 2.21-1.97 (m, 7 H), 1.47 (d, J = |
| Example 137 | | 5.6 Hz, 6H) |
| 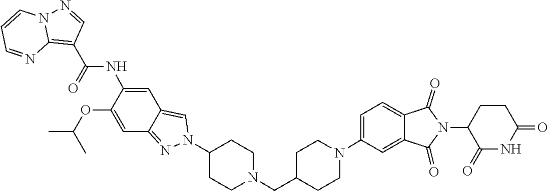 [SMILES: CC(C)Oc1cc2nn(C3CCN(CC4CCN(c5ccc6c(c5)C(=O)N(C5CCC(=O)NC5=O)C6=O)CC4)CC3)cc2cc1NC(=O)c1cnn2cccnc12] | 773.2 | DMSO-d₆ δ = 11.07 (s, 1H), 10.58 (s, 1H), 9.36 (dd, J = 1.6, 7.2 Hz, 1H), 8.86 (dd, J = 1.6, 4.4 Hz, 1H), 8.75 (s, 1H), 8.70 (s, 1H), 8.30 (s, 1H), 8.21 (s, 1H), 7.64 (d, J = 8.4 Hz, 1H), 7.36-7.27 (m, 2H), 7.22 (dd, J = 2.0, 8.8 Hz, 1H), 7.13 (s, 1H), 5.07 (dd, J = 5.2, 12.8 Hz, 1H), 4.80-4.90 (m, 1H), 4.35 (br s, 1H), 4.08-3.98 (m, 3H), 3.02-2.92 (m, 4H), 2.91-2.83 (m, 1H), 2.64-2.54 (m, 2H), 2.20 (br d, J = 6.4 Hz, 2H), |
| Example 138 | | 2.13-1.98 (m, 7H), 1.81 (br d, J = 10.8 |
| | | Hz, 3H), 1.48 (d, J = 5.6 Hz, 6H), |
| | | 1.16 (q, J = 11.2 Hz, 2H) |
| 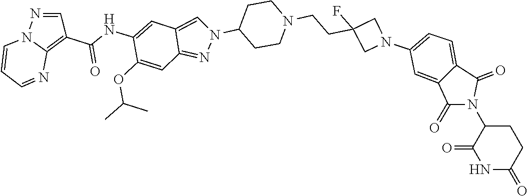 [SMILES: CC(C)Oc1cc2nn(C3CCN(CCC4(F)CN(c5ccc6c(c5)C(=O)N(C5CCC(=O)NC5=O)C6=O)C4)CC3)cc2cc1NC(=O)c1cnn2cccnc12] | 777.3 | DMSO-d₆ δ = 11.07 (s, 1H), 10.61 (s, 1H), 9.39 (d, J = 7.0 Hz, 1H), 8.88 (d, J = 4.0 Hz, 1H), 8.72 (d, J = 7.2 Hz, 1H), 8.32 (s, 1H), 8.19 (s, 1H), 7.63 (d, J = 8.4 Hz, 1H), 7.34 (dd, J = 4.4, 7.2 Hz, 1H), 6.91 (s, 1H), 6.78 (s, 1H), 6.65 (br d, J = 8.4 Hz, 1H), 5.06 (dd, J = 5.2, 12.8 Hz, 1H), 4.92 (t, J = 6.8 Hz, 1H), 4.37 (br t, J = 7.6 Hz, 1H),4.15 (br t, J = 8.0 Hz, 2H), 3.74-3.70 (m, 2H), 2.98 (br d, J = 10.8 Hz, 2H), |
| Example 139 | | 2.90-2.83 (m, 1H), 2.67 (br d, J = |
| | | 6.0 Hz, 2H), 2.65-2.61 (m, 2H), |
| | | 2.61 (br s, 1H), 2.54 (s, 1H), 2.33- |
| | | 2.25 (m, 3H), 2.21-2.15 (m, 2H), |
| | | 2.07 (brs, 5H), 1.96- 1.89 (m, 1H), |
| | | 1.82-1.75 (m, 1H) |
| 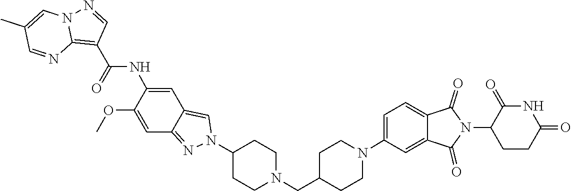 [SMILES: COc1cc2nn(C3CCN(CC4CCN(c5ccc6c(c5)C(=O)N(C5CCC(=O)NC5=O)C6=O)CC4)CC3)cc2cc1NC(=O)c1cnn2cc(C)cnc12] | 759.2 | Methanol-d δ = 10.44 (s, 1H), 8.85 (s, 1H), 8.71 (s, 1H), 8.65-8.61 (m, 2H), 8.07 (br s, 1H), 7.88 (s, 1H), 7.69 (d, J = 8.4 Hz, 1H), 7.30 (d, J = 2.4 Hz, 1H), 7.08-7.04 (m, 2H), 4.95 (dd, J = 5.6, 12.2 Hz, 1H), 4.38 (br d, J = 6.0 Hz, 1H), 4.05 (s, 3H), 3.98 (br d, J = 12.8 Hz, 2H), 3.08 (br s, 2H), 3.00 (br t,J = 12 Hz, 2H), 2.94-2.87 (m, 1H), 2.86- 2.68 (m, 2H), 2.49 (s, 3H), 2.34 (br d, J = 6.4 Hz, 2H), 2.27 (br s, 6H), |
| Example 140 | | 2.14 (br dd, J = 5.4, 8.0 Hz, 1H), |
| | | 1.94 (br d, J = 13.6 Hz, 2H), 1.85 (br |
| | | s, 1H), 1.39-1.23 (m, 3H) |
| 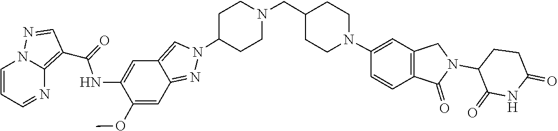 [SMILES: COc1cc2nn(C3CCN(CC4CCN(c5ccc6c(c5)CN(C5CCC(=O)NC5=O)C6=O)CC4)CC3)cc2cc1NC(=O)c1cnn2cccnc12] | 731.3 | MeOD δ = 10.75 (s, 1H), 9.14 (dd, J = 1.6, 6.8 Hz, 1H), 8.90 (dd, J = 1.6, 4.0 Hz, 1H), 8.74 (s, 1H), 8.21 (s, 1H), 7.65 (d, J = 8.8 Hz, 1H), 7.27 (dd, J = 4.0, 7.2 Hz, 1H),7.16- 7.04 (m, 3H), 5.11 (dd, J = 5.6, 13.2 Hz, 1H), 4.84-4.75 (m, 1H), 4.49-4.34 (m, 2H), 4.10 (s, 3H), |
| Example 141 | | 4.01 (br d, J = 13.2 Hz, 2H), 3.87 |
| | | (br d, J = 12.4 Hz, 2H), 3.62 (br s, |
| | | 1H), 3.29-3.24 (m, 1H), 3.18 (br |
| | | d, J = 6.8 Hz, 2H), 3.03-2.86 (m, |
| | | 3H), 2.83-2.74 (m, 1H), 2.58- |
| | | 2.40 (m, 5H), 2.24-2.11 (m, 2H), 1.96 |
| | | (br d, J = 10.8 Hz, 2H), 1.50 (m, 2H) |
| 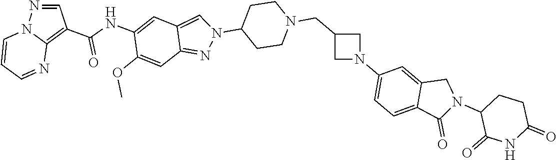 [SMILES: COc1cc2nn(C3CCN(CC4CN(c5ccc6c(c5)CN(C5CCC(=O)NC5=O)C6=O)C4)CC3)cc2cc1NC(=O)c1cnn2cccnc12] | 703.3 | CDCl₃ δ : 10.35-10.57 (m, 1 H) 8.81-8.89 (m, 2 H) 8.72-8.81 (m, 2 H) 7.86-7.97 (m, 2 H) 7.66-7.74 (m, 1 H) 7.04-7.10 (m, 2 H) 6.43-6.51 (m, 1H) 6.35-6.42 (m, 1 H) 5.12-5.30 (m, 1 H) 4.33- 4.43 (m, 2 H) 4.20-4.29 (m, 1 H) 4.09-4.17 (m, 2 H) 4.05-4.07 (m, 3 H) 3.64-3.70 (m, 2 H) 2.96-3.17 (m, 3 H) 2.86-2.92 (m, 1 H) 2.69-2.93 |
| Example 142 | | (m, 4 H) 2.26-2.29 (m, 1 H) 2.22-2.30 |
| | | (m, 3 H) 1.21-1.32 (m, 3 H) |
| 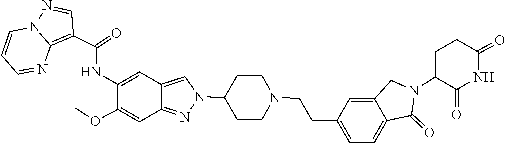 [SMILES: COc1cc2nn(C3CCN(CCc4ccc5c(c4)CN(C4CCC(=O)NC4=O)C5=O)CC3)cc2cc1NC(=O)c1cnn2cccnc12] | 662.2 | CDCl₃ δ = 10.71 (s, 1 H) 8.84-8.87 (m, 2 H) 8.80 (s, 1 H) 8.73 (dd, J = 4.0, 1.6 Hz, 1 H) 7.93 (s, 1 H) 7.73 (d, J = 8.4 Hz, 1 H) 7.39 (s, 1 H) 7.09 (dd, J = 6.8, 4.0 Hz, 1 H) 7.01 (dd, J = 8.4, 2.0 Hz, 1 H) 6.90 (s, 1 H) 5.18-5.24 (m, 1 H) 4.75- 4.85 (m, 1 H) 4.38-4.46 (m, 1 H) 4.34 (s, 2 H) 4.23-4.31 (m, 2 H) |
| Example 143 | | 3.80-3.90 (m, 2 H) 3.47-3.52 (m, |
| | | 1 H) 2.93-3.04 (m, 2 H) 2.84- |
| | | 2.92 (m, 3 H) 2.31-2.41 (m, 1 H) |
| | | 2.10-2.30 (m, 5 H) 1.83-1.93 (m, |
| | | 5 H) 1.67-1.79 (m, 1 H) 1.52 (d, |
| | | J = 6.0 Hz, 6 H) 1.27-1.41 (m, 3 H) |
| 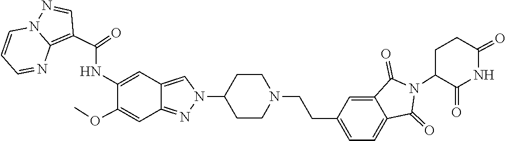 [SMILES: COc1cc2nn(C3CCN(CCc4ccc5c(c4)C(=O)N(C4CCC(=O)NC4=O)C5=O)CC3)cc2cc1NC(=O)c1cnn2cccnc12] | 676.2 | CDCl₃ δ = 10.46 (s, 1H), 8.85 (s, 1H), 8.83 (dd, J = 2.0, 7.2 Hz, 1H), 8.80 (s, 1H), 8.76 (dd, J = 1.6, 4.4 Hz, 1H), 8.10 (s, 1H), 7.89 (s, 1H), 7.83 (d, J = 7.6 Hz, 1H), 7.77 (s, 1H), 7.63 (br d, J = 7.6 Hz, 1H), 7.08-7.03 (m, 2H), 5.00 (dd, J = 5.2, 12.4 Hz, 1H), 4.43-4.31 (m, 1H), 4.06 (s, 3H), 3.20-3.10 (m, |
| Example 144 | | 2H), 3.02-2.95 (m, 2H), 2.92- |
| | | 2.80 (m, 2H), 2.76-2.71 (m, 2H), |
| | | 2.34-2.15 (m, 8H) |
| 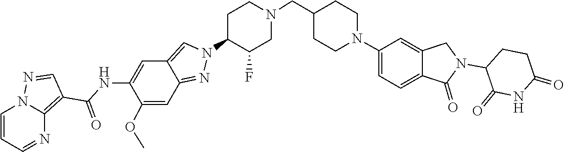 [SMILES: COc1cc2nn([C@H]3CCN(CC4CCN(c5ccc6c(c5)CN(C5CCC(=O)NC5=O)C6=O)CC4)C[C@@H]3F)cc2cc1NC(=O)c1cnn2cccnc12] | 749.2 | CDCl₃ δ : 10.24-10.59 (m, 1 H) 8.81-8.87 (m, 2 H) 8.74-8.81 (m, 2 H) 8.00-8.09 (m, 1 H) 7.90- 7.96 (m, 1 H) 7.61-7.82 (m, 1 H) 7.08-7.11 (m, 1 H) 6.98-7.08 (m, 2 H) 6.86-6.93 (m, 1 H) 5.17- 5.28 (m, 1 H) 4.94-5.07 (m, 1 H) 4.82-4.91 (m, 1 H) 4.39-4.50 (m, 1 H) 4.30-4.38 (m, 1 H) 4.21- 4.29 (m, 1 H) 4.01-4.12 (m, 3 H) |
| Example 145 | | 3.81-3.94 (m, 2 H) 3.30-3.41 (m, |
| | | 1 H) 2.99-3.10 (m, 1 H) 2.88- |
| | | 2.96 (m, 2 H) 2.87 (br s, 2 H) 2.39- |
| | | 2.55 (m, 2 H) 2.32-2.38 (m, 2 H) |
| | | 2.23-2.30 (m, 2 H) 1.89-1.97 (m, 2 H) |
| | | 1.74-1.81 (m, 2 H) 1.29-1.39 (m, 2H) |
| 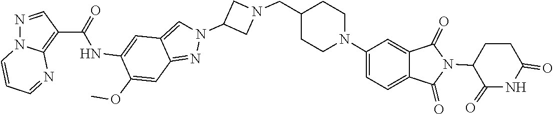 [SMILES: COc1cc2nn(C3CN(CC4CCN(c5ccc6c(c5)C(=O)N(C5CCC(=O)NC5=O)C6=O)CC4)C3)cc2cc1NC(=O)c1cnn2cccnc12] | 717.2 | DMSO-d₆ δ = 11.07 (s, 1H), 10.55 (s, 1H), 9.38 (br d, J = 7.2 Hz, 1H), 8.97-8.94 (m, 1H), 8.72-8.67 (m, 2H), 8.38 (s, 1H), 7.65 (d, J = 8.4 Hz, 1H), 7.35 (dd, J = 4.8, 6.8 Hz, 1H), 7.31 (br s, 1H), 7.23 (br d, J = 8.8 Hz, 1H), 7.15 (s, 1H), 5.21 (br |
| Example 146 | | t, J = 6.8 Hz, 1H), 5.06 (dd,J = 5.2, |
| | | 12.8 Hz, 1H), 4.09-3.99 (m, 5H), |
| | | 3.80 (br t, J = 7.2 Hz, 2H), 3.52 (br |
| | | t, J = 7.2 Hz, 2H), 3.00-2.83 (m, |
| | | 3H), 2.62-2.54 (m, 2H), 2.45 (br |
| | | d, J = 6.8 Hz, 2H), 2.06-1.97 (m, |
| | | 1H), 1.86-1.76 (m, 2H), 1.71- |
| | | 1.58 (m, 1H), 1.27-1.14 (m, 2H) |
| 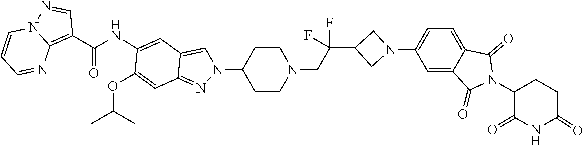 [SMILES: CC(C)Oc1cc2nn(C3CCN(CC(F)(F)C4CN(c5ccc6c(c5)C(=O)N(C5CCC(=O)NC5=O)C6=O)C4)CC3)cc2cc1NC(=O)c1cnn2cccnc12] | 795.2 | DMSO-d₆ δ = 11.07 (s, 1 H) 10.59 (s, 1 H) 9.38 (dd, J = 6.8, 1.6 Hz, 1 H) 8.87 (dd, J = 4.4, 1.6 Hz, 1 H) 8.75 (s, 1 H) 8.71 (s, 1 H) 8.31 (s, 1 H) 7.67 (d, J = 8.4 Hz, 1 H) 7.34 (dd, J = 6.8, 4.0 Hz, 1 H) 7.14 (s, 1 H) 6.88 (s, 1 H) 6.73 (dd, J = 8.0,1.6 Hz, 1 H) 5.06 (dd, J = 12.4, 5.2 Hz, 1 H) 4.86 (dt, J = 12.0, 6.0 Hz, 1 H) |
| Example 147 | | 4.36 (m, 1 H) 4.20-4.26 (m, 2 H) |
| | | 4.03-4.09 (m, 2 H) 3.52-3.62 (m, |
| | | 1 H) 2.83-3.03 (m, 5 H) 2.53- |
| | | 2.68 (m, 2 H) 2.44 (br s, 1 H) 1.92- |
| | | 2.21 (m, 6 H) 1.48 (d, J = 6.0 Hz, 6 |
| | | H) |
| 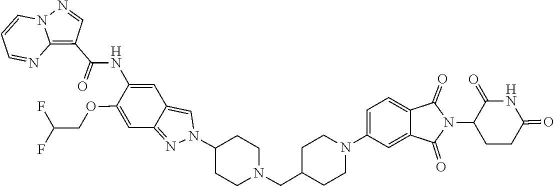 [SMILES: O=C1CCC(N2C(=O)c3ccc(N4CCC(CN5CCC(n6cc7cc(NC(=O)c8cnn9cccnc89)c(OCC(F)F)cc7n6)CC5)CC4)cc3C2=O)C(=O)N1] | 795.2 | DMSO-d₆ δ = 11.08 (s, 1H), 10.60 (s, 1H), 9.38 (dd, J = 1.6, 7.2 Hz, 1H), 8.86 (dd, J = 1.6, 4.0 Hz, 1H), 8.77 (s, 1H), 8.71 (s, 1H), 8.36 (s, 1H), 8.28 (s, 1H), 7.65 (d, J = 8.4 Hz, 1H), 7.38-7.34 (m, 1H), 7.32 (d, J = 2.0 Hz, 1H), 7.26-7.19 (m, 2H), 6.80-6.47 (m, 1H), 5.06 (dd, J = 5.2, 12.8 Hz, 1H), 4.52 (dt, J = 3.6, 14.4 Hz, 2H), 4.43-4.33 (m, 1H), 4.06 (br d, J = 13.2 Hz, 2H), 2.98 (br t, J = 11.2 Hz, 4H), 2.90- |
| Example 148 | | 2.82 (m, 1H), 2.60 (br d, J = 2.8 Hz, |
| | | 1H), 2.56 (br dd, J = 3.2, 9.6 Hz, |
| | | 1H), 2.21 (br d, J = 6.8 Hz, 2H), |
| | | 2.14-2.05 (m, 6H), 2.04-1.96 (m, |
| | | 1H), 1.83 (br d, J = 11.6 Hz, 3H), |
| | | 1.17 (br d, J = 12.0 Hz, 2H) |
| 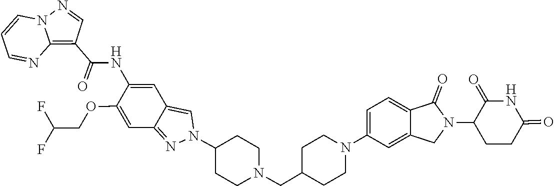 [SMILES: O=C1CCC(N2Cc3cc(N4CCC(CN5CCC(n6cc7cc(NC(=O)c8cnn9cccnc89)c(OCC(F)F)cc7n6)CC5)CC4)ccc3C2=O)C(=O)N1] | 781.4 | DMSO-d₆ δ = 10.95 (s, 1H), 10.60 (s, 1H), 9.38 (dd, J = 1.2, 7.2 Hz, 1H), 8.90-8.83 (m, 1H), 8.77 (s, 1H), 8.71 (s, 1H), 8.36 (s, 1H), 8.28 (s, 1H), 7.50 (d, J = 8.8 Hz, 1H), 7.36 (dd, J = 4.0, 7.2 Hz, 1H), 7.22 (s, 1H), 7.09-7.01 (m, 2H), 6.82- 6.46 (m, 1H), 5.04 (br dd, J = 5.2, 13.2 Hz, 1H),4.52 (dt, J = 3.2,14.0 Hz, 2H), 4.39-4.13 (m, 3H), 3.89 (br d, J = 12.0 Hz, 2H), 2.98 (br d, J = 3.2 Hz, 2H), 2.89-2.76 (m, |
| Example 149 | | 3H), 2.36 (br dd, J = 4.4, 12.8 Hz, |
| | | 1H), 2.22 (br d, J = 6.4 Hz, 2H), |
| | | 2.09 (br d, J = 5.2 Hz, 6H), 2.00- |
| | | 1.91 (m, 1H), 1.81 (br d, J = 11.2 |
| | | Hz, 3H), 1.20 (br d, J = 12.0 Hz, 2H) |
| 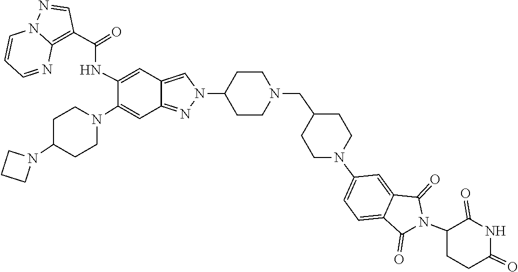 [SMILES: O=C1CCC(N2C(=O)c3ccc(N4CCC(CN5CCC(n6cc7cc(NC(=O)c8cnn9cccnc89)c(N8CCC(N9CCC9)CC8)cc7n6)CC5)CC4)cc3C2=O)C(=O)N1] | 853.4 | DMSO-d₆ δ = 11.08 (s, 1 H) 10.70 (s, 1 H) 9.38 (d, J = 6.0 Hz, 1 H) 8.96 (br d, J = 2.4 Hz, 1 H) 8.74 (d, J = 18.8 Hz, 2 H) 8.34 (s, 1 H) 8.21 (s, 1 H) 7.65 (d, J = 8.4 Hz, 1 H) 7.36-7.46 (m, 2 H) 7.31 (s, 1 H) 7.24 (br d, J = 8.4 Hz, 1 H) 5.07 (br dd, J = 12.8, 5.6 Hz, 1 H) 4.40 (br s, l H) 4.06 (br d, J = 12.4 Hz, 2 H) 3.29 (br s, 3 H) 2.92-3.09 (m, 8 H) 2.83-2.90 (m, 1 H) 2.71 (m, 2 H) 2.61 (br s, 2 H) 2.34 (m, 2 H) 2.21 (m, 2 H) 2.01-2.14 (m, 8 H) 1.82 (br d, J = 10.4 Hz, 4 H) 1.63 (br d, J = 9.6 Hz, 2 H) 1.17 (br d, J = 12.4 Hz, 2 H) |
| Example 150 | | |
| 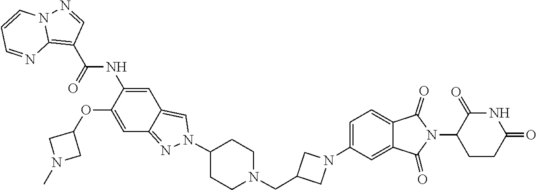 [SMILES: CN1CC(Oc2cc3nn(C4CCN(CC5CN(c6ccc7c(c6)C(=O)N(C6CCC(=O)NC6=O)C7=O)C5)CC4)cc3cc2NC(=O)c2cnn3cccnc23)C1] | 772.3 | DMSO-d₆ δ = 11.07 (br s, 1H), 10.57 (s, 1H), 9.39 (dd, J = 1.6, 7.2 Hz, 1H), 8.89 (dd, J = 1.6, 4.4 Hz, 1H), 8.73 (d, J = 9.2 Hz, 2H), 8.33 (s, 1H), 7.63 (d, J = 8.4 Hz, 1H), 7.36 (dd, J = 4.0, 6.8 Hz, 1H), 6.87 (s, 1H), 6.77 (d, J = 1.6 Hz, 1H), 6.64 (dd, J = 2.0, 8.4 Hz, 1H), 5.06 (dd, J = 5.2, 12.8 Hz, 1H), 5.01- 4.94 (m, 1H), 4.41-4.32 (m, 1H), 4.15 (br t, J = 8.0 Hz, 2H), 3.94 (dd, J = 6.4, 8.4 Hz, 2H), 3.71 (br dd, J = |
| Example 151 | | 6.0, 8.0 Hz, 2H), 3.23-3.21 (m, |
| | | 2H), 3.04-2.95 (m, 3H), 2.94- |
| | | 2.83 (m, 2H), 2.69-2.66 (m, 2H), |
| | | 2.69-2.65 (m, 1H), 2.37 (s, 3H), |
| | | 2.22-2.15 (m,2H),2.11-1.99 (m, 6H) |
| 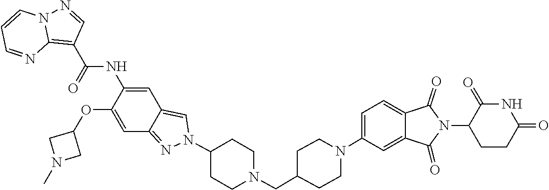 [SMILES: CN1CC(Oc2cc3nn(C4CCN(CC5CCN(c6ccc7c(c6)C(=O)N(C6CCC(=O)NC6=O)C7=O)CC5)CC4)cc3cc2NC(=O)c2cnn3cccnc23)C1] | 800.3 | DMSO-d₆ δ = 11.08 (s, 1 H) 10.57 (s, 1 H) 9.39 (d, J = 7.2 Hz, 1 H) 8.90 (d, J = 2.8 Hz, 1 H) 8.73 (d, J = 7.2 Hz, 2 H) 8.33 (s, 1 H) 8.18 (s, 1 H) 7.65 (d, J = 8.8 Hz, 1 H) 7.36 (dd, J = 6.4, 4. 0 Hz, 1 H) 7.31 (s, 1 H) 7.24 (br d, J = 8.4 Hz, 1 H) 6.86 (s, 1 H) 5.06 (dd, J = 12.8, 5.2 Hz, 1 H) 4.95-5.01 (m, 1 H) 4.36 (br s, 1 H) 4.05 (br d, J = 12.4 Hz, 2 H) 3.95 (m, 2 H) 3.24 (br s, 1 H) 2.94-3.03 (m, 5 H) 2.82-2.93 (m, |
| Example 152 | | 2 H) 2.59-2.69 (m, 1 H) 2.56 (m, |
| | | 1 H) 2.38 (s, 3 H) 2.21 (br d, J = 6.0 |
| | | Hz, 2 H) 2.05-2.13 (m, 6 H) 1.82 |
| | | (br d, J = 12.8 Hz, 3 H) 1.17 (br d, |
| | | J = 12.4 Hz, 2 H) |
| 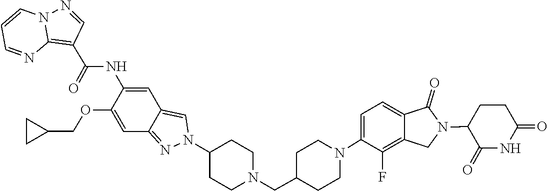 [SMILES: O=C1CCC(N2Cc3c(ccc(N4CCC(CN5CCC(n6cc7cc(NC(=O)c8cnn9cccnc89)c(OCC8CC8)cc7n6)CC5)CC4)c3F)C2=O)C(=O)N1] | 789.3 | DMSO-d₆ δ = 10.98 (s, 1 H) 10.65 (s, 1 H) 9.37 (dd, J = 6.8, 1.6 Hz, 1 H) 8.86 (dd, J = 4.0, 1.6 Hz, 1 H) 8.72 (d, J = 11.6 Hz, 2 H) 8.30 (s, 1 H) 7.46 (d, J = 8.4 Hz, 1 H) 7.31 (dd, J = 6.8, 4.0 Hz, 1 H) 7.16 (1, J = 7.6 Hz, 1 H) 7.03 (s, 1 H) 5.07 (dd, J = 13.6, 4.8 Hz, 1 H) 4.27-4.56 (m, 4 H) 4.02 (d, J = 7.2 Hz, 2 H) 2.85- 3.05 (m, 4H) 2.79 (br t, J = 11.6 Hz, 2 H) 2.57-2.68 (m, 3 H) 2.42 (br dd, J = 12.4, 4.4 Hz, 1 H) 2.25 (br d, |
| Example 153 | | J = 6.8 Hz, 2 H) 2.09 (m, 4 H) 1.94- |
| | | 2.00 (m, 1H) 1.84 (br d, J = 11.6 Hz, |
| | | 2 H) 1.71 (br s, 1 H) 1.49 (br s, 1 |
| | | H) 1.29 (q, J = 10.8 Hz, 2 H) 0.67- |
| | | 0.76 (m, 2 H) 0.45 (m, 2 H) |
| 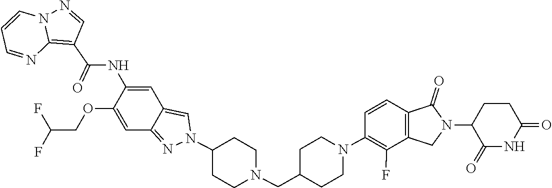 [SMILES: O=C1CCC(N2Cc3c(ccc(N4CCC(CN5CCC(n6cc7cc(NC(=O)c8cnn9cccnc89)c(OCC(F)F)cc7n6)CC5)CC4)c3F)C2=O)C(=O)N1] | 799.3 | DMSO-d₆ δ = 10.98 (s, 1 H) 10.59 (s, 1 H) 9.38 (dd, J = 7.2, 1.6 Hz, 1 H) 8.85 (dd, J = 4.4, 1.6 Hz, 1 H) 8.69-8.79 (m, 2 H) 8.35 (s, 1 H) 7.47 (d, J = 8.4 Hz, 1 H) 7.35 (dd, J = 7.2, 4.4 Hz, 1 H) 7.22 (s, 1 H) 7.16 (br t, J = 7.6 Hz, 1 H) 6.47- 6.80 (m, 1 H) 5.07 (dd, J = 13.2, 4.8 Hz, 1 H) 4.29-4.56 (m, 6 H) 2.84- 3.01 (m, 4 H) 2.79 (br t, J = 11.2 Hz, 2 H) 2.56-2.68 (m, 3 H) 2.39- 2.44 (m, 1 H) 2.25 (br d, J = 7.2 |
| Example 154 | | Hz, 2 H) 2.10 (br d, J = 5.6 Hz, 4 H) |
| | | 1.95-2.00 (m, 1 H) 1.84 (br d, |
| | | J = 11.2 Hz, 2 H) 1.72 (br s, 1 H) |
| | | 1.25-1.34 (m, 2H) |
| 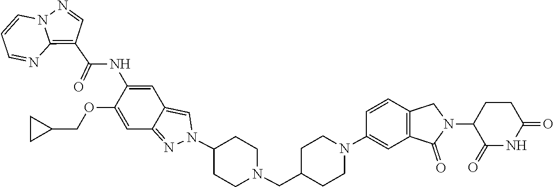 [SMILES: O=C1CCC(N2Cc3ccc(N4CCC(CN5CCC(n6cc7cc(NC(=O)c8cnn9cccnc89)c(OCC8CC8)cc7n6)CC5)CC4)cc3C2=O)C(=O)N1] | 771.5 | DMSO-d₆ δ = 10.97 (br s, 1 H) 10.64 (s, 1 H) 9.37 (dd, J = 6.80, 1.60 Hz, 1 H) 8.86 (dd, J = 4.00, 1.60 Hz, 1 H) 8.74 (s, 1 H) 8.71 (s, 1 H) 8.30 (s, 1 H) 7.41 (d, J = 8.40 Hz, 1 H) 7.31 (dd, J = 7.20, 4.40 Hz, 1 H) 7.22-7.28 (m, 1 H) 7.16 (d, J = 2.00 Hz, 1 H) 7.03 (s, 1 H) 5.09 (dd, J = 13.20, 5.20 Hz, 1 H) 4.28- 4.40 (m, 2 H) 4.15-4.24 (m, 1 H) 4.02 (d, J = 6.80 Hz, 2 H) 3.76 (br d, J = 11.20 Hz, 2 H) 2.85-3.00 (m, 3 |
| Example 155 | | H) 2.72 (br t, J = 11.20 Hz, 2 H) 2.57 |
| | | (br s, 1 H) 2.38 (br dd, J = 13.40, |
| | | 4.28 Hz, 1 H) 2.22 (br d, J = 7.20 |
| | | Hz, 2 H) 2.04-2.12 (m, 6 H) 1.95- |
| | | 2.03 (m, 1 H) 1.82 (br d, J = 12.20 |
| | | Hz, 2 H) 1.71 (br dd, J = 7.20, 4.00 |
| | | Hz, 1 H) 1.44-1.55 (m, 1 H) 1.17- |
| | | 1.31 (m, 2 H) 0.64-0.76 (m, 2 |
| | | H) 0.46 (q, J = 4.40 Hz, 2 H) |
| 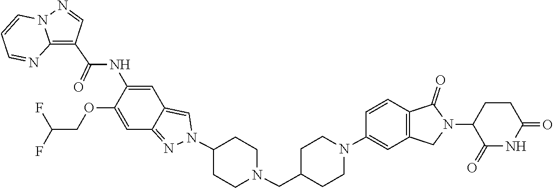 [SMILES: O=C1CCC(N2Cc3cc(N4CCC(CN5CCC(n6cc7cc(NC(=O)c8cnn9cccnc89)c(OCC(F)F)cc7n6)CC5)CC4)ccc3C2=O)C(=O)N1] | 781.2 | DMSO-d₆ δ = 10.97 (s, 1 H) 10.60 (s, 1 H) 9.39 (dd, J = 7.2, 1.6 Hz, 1 H) 8.86 (dd, J = 4.0, 1.6 Hz, 1 H) 8.77 (s, 1 H) 8.72 (s, 1 H) 8.37 (s, 1 H) 7.41 (d, J = 8.8 Hz, 1 H) 7.34- 7.38 (m, 1 H) 7.26 (dd, J = 8.4, 2.0 Hz, 1 H) 7.23 (s, 1 H) 7.16 (d, J = 2.0 Hz, 1 H) 6.48-6.80 (m, 1 H) 5.06-5.14 (m, 1 H) 4.53 (td, J = 14.0,3.6 Hz, 2 H) 4.37-4.43 (m, 1 H) 4.18-4.35 (m, 2 H) 3.77 (br d, J = 12.4 Hz, 2 H) 2.99 (brd, J = 3.6 |
| Example 156 | | Hz, 2 H) 2.86-2.95 (m, 1 H) 2.73 |
| | | (br t, J = 11.2 Hz, 2 H) 2.56-2.63 |
| | | (m, 1 H) 2.35-2.42 (m, 1 H) 2.23 |
| | | (br d, J = 7.2 Hz, 2 H) 2.06-2.13 |
| | | (m, 6 H) 1.96-2.02 (m, 1 H) 1.83 |
| | | (br d, J = 12.0 Hz, 2 H) 1.68-1.77 |
| | | (m, 1 H) 1.19-1.30 (m, 2 H) |
| 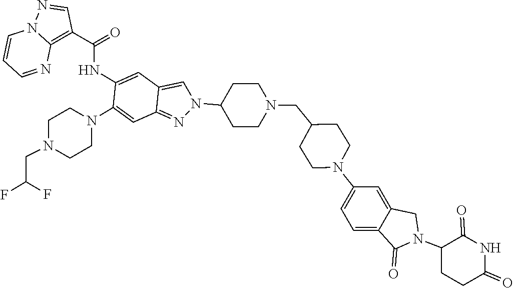 [SMILES: O=C1CCC(N2Cc3cc(N4CCC(CN5CCC(n6cc7cc(NC(=O)c8cnn9cccnc89)c(N8CCN(CC(F)F)CC8)cc7n6)CC5)CC4)ccc3C2=O)C(=O)N1] | 849.3 | DMSO-d₆ δ = 10.96 (s, 1 H) 10.63 (s, 1 H) 9.39 (dd, J = 6.8, 1.2 Hz, 1 H) 8.98 (dd, J = 4.0, 1.6 Hz, 1 H) 8.76 (s, 1 H) 8.72 (s, 1 H) 8.36 (s, 1 H) 7.50 (d, J = 9.2 Hz, 1 H) 7.45 (s, 1 H) 7.34-7.40 (m, 1 H) 7.03- 7.07 (m, 2 H) 6.05-6.38 (m, 1 H) 5.00-5.10 (m, 1 H) 4.36-4.46 (m, 1 H) 4.17-4.34 (m, 2 H) 3.89 (m, 2 H) 2.80-3.00 (m, 16 H) 2.55- 2.61 (m, 1 H) 2.22 (m, 2 H) 2.09 (br s, 6 H) 1.92-1.98 (m, 1H) 1.81 (m, 3 H) 1.16-1.26 (m, 2 H) |
| Example 157 | | |
| 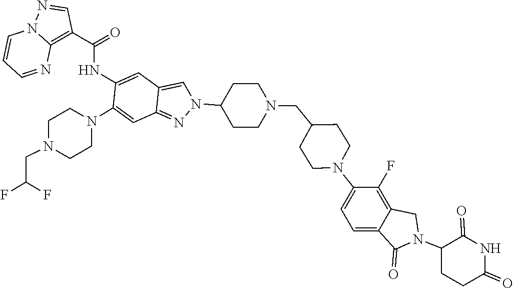 [SMILES: O=C1CCC(N2Cc3c(ccc(N4CCC(CN5CCC(n6cc7cc(NC(=O)c8cnn9cccnc89)c(N8CCN(CC(F)F)CC8)cc7n6)CC5)CC4)c3F)C2=O)C(=O)N1] | 867.3 | DMSO-d₆ δ = 11.00 (s, 1 H) 10.63 (s, 1 H) 9.39 (dd, J = 6.8, 1.6 Hz, 1 H) 8.98 (dd, J = 4.4, 1.6 Hz, 1 H) 8.74 (d, J = 14.0 Hz, 2 H) 8.36 (s, 1 H) 8.12-8.18 (m, 1H) 7.42-7.49 (m, 2 H) 7.35-7.40 (m, 1 H) 7.17 (t, J = 8.0 Hz, 1 H) 6.06-6.38 (m, 1 H) 5.08 (dd, J = 13.2, 5.2 Hz, 1 H) 4.29-4.51 (m, 3 H) 3.50 (br s, 2 H) 2.76-3.00 (m, 16 H) 2.56-2.62 (m, 1 H) 2.26 (m, 2 H) 2.06-2.15 (m, 6 H) 1.95-2.01 (m, 1 H) 1.85 (m, 2 H) 1.67-1.76 (m, 1 H) 1.30 (q, J = 10.8 Hz, 2H) |
| Example 158 | | |
| 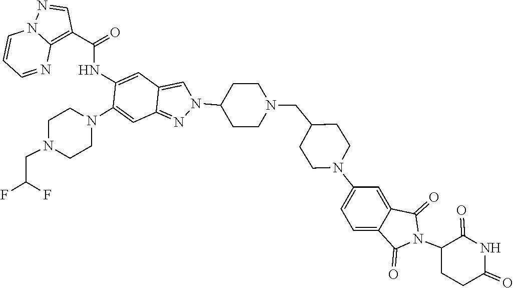 [SMILES: O=C1CCC(N2C(=O)c3ccc(N4CCC(CN5CCC(n6cc7cc(NC(=O)c8cnn9cccnc89)c(N8CCN(CC(F)F)CC8)cc7n6)CC5)CC4)cc3C2=O)C(=O)N1] | 863.3 | DMSO-d₆ δ = 11.07 (s, 1 H), 10.62 (s, 1 H), 9.38 (m, 1 H), 8.98 (m, 1 H), 8.76 (s, 1 H), 8.72 (s, 1 H), 8.35 (s, 1 H), 7.65 (d, J = 8.4 Hz, 1 H), 7.45 (s, 1 H), 7.37 (s, 1 H), 7.32 (d, J = 1.6 Hz, 1H), 7.24 (s, 1H),6.06- 6.37 (m, 1 H), 5.01-5.12 (m, 1 H), 4.32-4.46 (m, 1 H), 4.06 (d, J = 13.2 Hz, 2 H), 2.83-3.00 (m, 14 H), 2.52-2.62 (m, 2 H), 2.22 (d, J = 6.8 Hz, 2 H), 1.98-2.16 (m, 8 H), 1.77- 1.88 (m, 3H), 1.11-1.26 (m, 2 H) |
| Example 159 | | |

### Preparation of Example 160: N-(6-(cyclopropylmethoxy)-2-(1-(2-(4-(2-(2,6-dioxopiperidin-3-yl)-1-oxoisoindolin-5-yl)piperazin-1-yl)ethyl)piperidin-4-yl)-2H-indazol-5-yl)pyrazolo[1,5-a]pyrimidine-3-carboxamide

### Step 1:

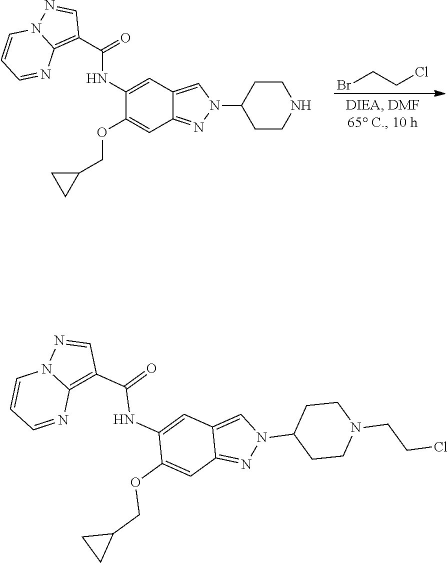

To a solution of N-(6-(cyclopropylmethoxy)-2-(piperidin-4-yl)-2H-indazol-5-yl)pyrazolo[1,5-a]pyrimidine-3-carboxamide (0.2 g, 463.5 μmol, 1 eq) and 1-bromo-2-chloro-ethane (0.2 g, 1.39 mmol, 115.61 μL, 3.01 eq) in DMF (3 mL) was added diisopropylethylamine (179.7 mg, 1.39 mmol, 242.2 μL, 3 eq). The mixture was stirred at 65° C. for 10 h. The reaction mixture was concentrated under reduced pressure. The residue was diluted with water (10 mL) and extracted with ethyl acetate 30 mL (10 mL×3). The combined organic layers were washed with brine (5 mL×2), dried over anhydrous sodium sulfate, filtered, and concentrated under reduced pressure to afford N-(2-(1-(2-chloroethyl)piperidin-4-yl)-6-(cyclopropylmethoxy)-2H-indazol-5-yl)pyrazolo[1,5-a]pyrimidine-3-carboxamide (200 mg, 404.9 μmol, 87% yield) as a white solid which was used in the next step without further purification. MS (ESI) m/z: 494.3 [M+H]⁺.

### Step 2:

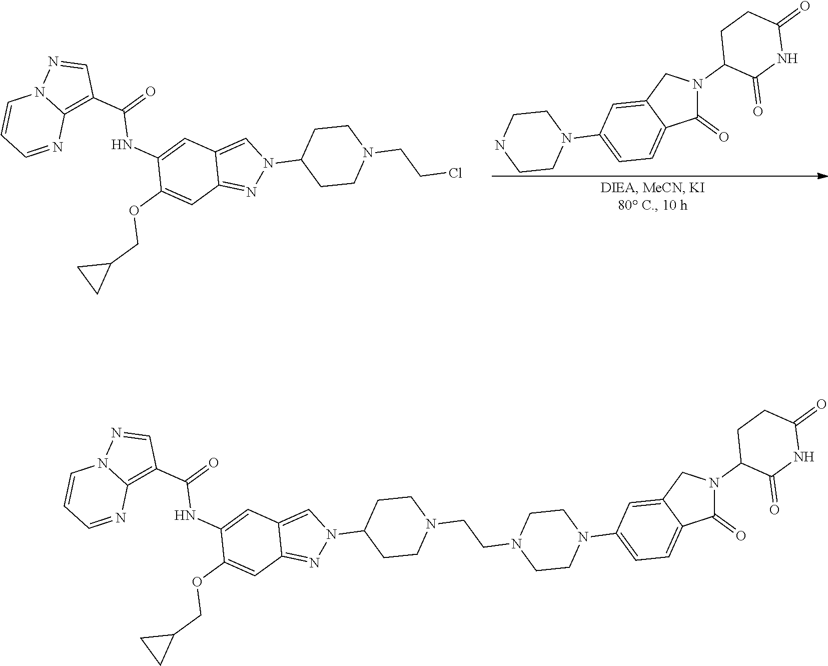

To a solution of N-(2-(1-(2-chloroethyl)piperidin-4-yl)-6-(cyclopropylmethoxy)-2H-indazol-5-yl)pyrazolo[1,5-a]pyrimidine-3-carboxamide (60 mg, 121.5 μmol, 1 eq) and 3-(1-oxo-5-(piperazin-1-yl)isoindolin-2-yl)piperidine-2,6-dione (44.3 mg, 121.5 μmol, 1.00 eq, hydrogen chloride) in acetonitrile (1 mL) was added potassium iodide (100.8 mg, 607.3 μmol, 5 eq) and diisopropylethylamine (109.9 mg, 850.2 μmol, 148.1 μL, 7 eq). The mixture was stirred at 80° C. for 10 h. The reaction mixture was concentrated under reduced pressure to remove solvent. The residue was purified by preparative HPLC (column: Phenomenex Luna C18 150×25 mm×10 m; mobile phase: [water (0.1% trifluoroacetic acid)-acetonitrile]; B %: 13%-43%, 10 min) to afford N-(6-(cyclopropylmethoxy)-2-(1-(2-(4-(2-(2,6-dioxopiperidin-3-yl)-1-oxoisoindolin-5-yl)piperazin-1-yl)ethyl)piperidin-4-yl)-2H-indazol-5-yl)pyrazolo[1,5-a]pyrimidine-3-carboxamide (12.3 mg, 15.0 μmol, 12% yield, 96% purity) as a pink solid. MS (ESI) m/z: 786.4 [M+H]⁺. ¹H NMR (400 MHz, methanol-d₄) δ=10.80 (s, 1H), 9.11 (dd, J=1.6, 7.0 Hz, 1H), 8.83 (dd, J=1.6, 4.0 Hz, 1H), 8.76 (s, 1H), 8.67 (s, 1H), 8.20 (s, 1H), 7.71-7.64 (m, 1H), 7.25 (dd, J=4.0, 7.1 Hz, 1H), 7.19-7.11 (m, 2H), 6.98 (s, 1H), 5.13-5.08 (m, 1H), 4.41 (d, J=5.6 Hz, 2H), 4.03 (d, J=6.4 Hz, 2H), 3.75 (br d, J=13.2 Hz, 2H), 3.53 (br s, 4H), 3.46 (br t, J=6.4 Hz, 2H), 3.24 (br s, 2H), 3.14 (br s, 4H), 2.98-2.70 (m, 3H), 2.60-2.36 (m, 5H), 2.21-2.09 (m, 1H), 2.03 (s, 1H), 1.61-1.46 (m, 1H), 1.40-1.27 (m, 1H), 0.80-0.70 (m, 2H), 0.54-0.48 (m, 2H).

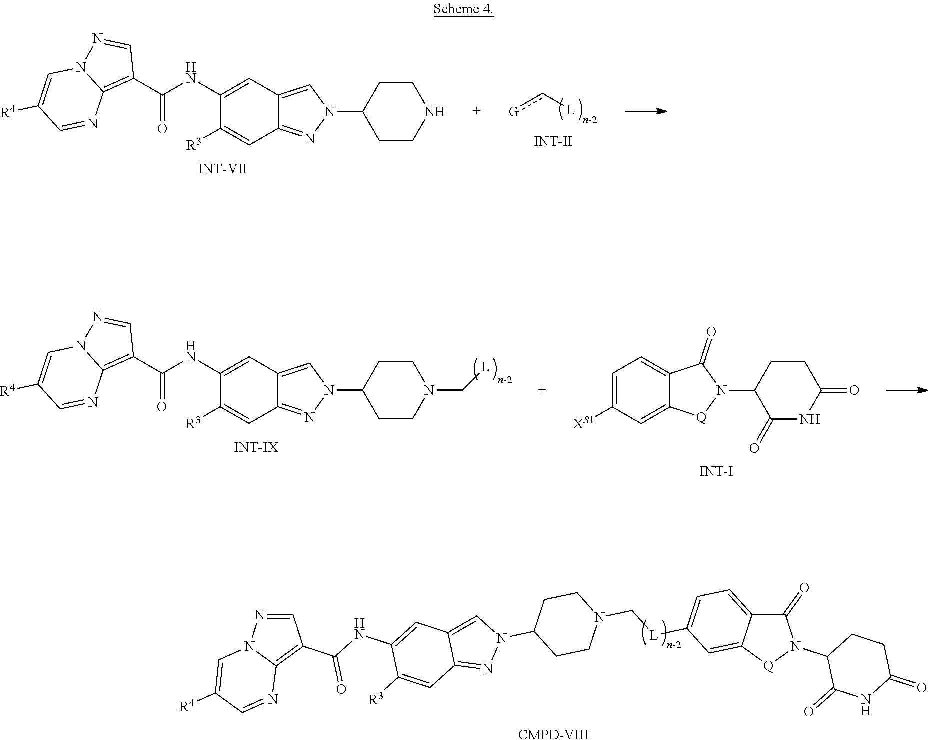

A compound of formula INT-VII, wherein R³ is an ether or alkyl group and R⁴ optionally substitutes as an H, an amine, methyl, or chloro group, may be reacted with a reagent INT-II (commercially available or readily prepared using standard reaction techniques known to one skilled in the art) under reductive amination or alkylation conditions to produce a compound of formula INT-IX, wherein L and n are as defined herein. Compounds of formula INT-IX can be furnished from N-alkylation where G is an appropriate leaving group (e.g., OMs, OTs, Cl, etc.) or through reductive amination where G is an aldehyde. When G is a leaving group, suitable reaction conditions are those for an alkylation reaction, e.g., diisopropylethylamine, potassium iodide, DMSO or acetonitrile, 80° C. When G is an aldehyde, suitable reaction conditions are those for a reductive amination reaction, e.g., sodium cyanoborohydride, methanol, dichloromethane, acetic acid, room temperature. Compounds of formula INT-IX can then be reacted with compounds of formula INT-I wherein Q is CH₂ or C═O and X^{S1} is an alcohol or a halide to produce compound of formula CVIPD-VIII.

### Preparation of Example 161: N-(6-(cyclopropylmethoxy)-2-(1-((1-(2-(2,6-dioxopiperidin-3-yl)-1,3-dioxoisoindolin-5-yl)-3-fluoroazetidin-3-yl)methyl)piperidin-4-yl)-2H-indazol-5-yl)pyrazolo[1,5-a]pyrimidine-3-carboxamide

### Step 1:

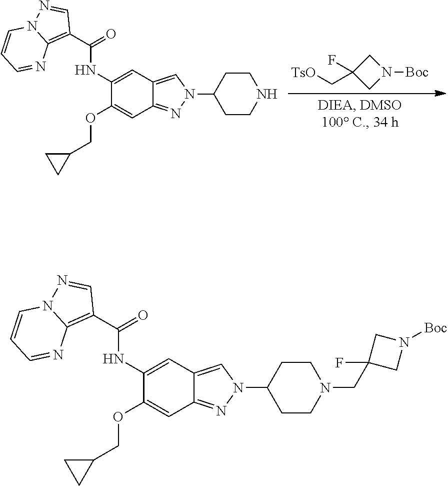 [SMILES: O=C(Nc1cc2cn(C3CCNCC3)nc2cc1OCC1CC1)c1cnn2cccnc12 ; Cc1ccc(S(=O)(=O)OCC2(F)CN(C(=O)OC(C)(C)C)C2)cc1 ; CC(C)(C)OC(=O)N1CC(F)(CN2CCC(n3cc4cc(NC(=O)c5cnn6cccnc56)c(OCC5CC5)cc4n3)CC2)C1]

To a mixture of N-(6-(cyclopropylmethoxy)-2-(piperidin-4-yl)-2H-indazol-5-yl)pyrazolo[1,5-a]pyrimidine-3-carboxamide (1.3 g, 3.01 mmol, 1 eq) and diisopropylethylamine (1.95 g, 15.09 mmol, 2.63 mL, 5.01 eq) in dimethyl sulfoxide (10 mL) was added tert-butyl 3-fluoro-3-((tosyloxy)methyl)azetidine-1-carboxylate (2.60 g, 7.23 mmol, 2.40 eq) at 0° C. The reaction mixture was then heated to 100° C. for 10 h under an atmosphere of nitrogen. To the mixture was added tert-butyl 3-fluoro-3-((tosyloxy)methyl)azetidine-1-carboxylate (1.30 g, 3.62 mmol, 1.2 eq) and then the mixture was stirred at 100° C. for 24 h under an atmosphere of nitrogen. The reaction was quenched with ice water (100 mL) and extracted with ethyl acetate (150 mL×3). The combined organic phases were washed with brine (200 mL), dried over anhydrous sodium sulfate, concentrated in vacuo to give a residue. The residue was purified by silica gel column chromatography (eluted with petroleum ether:ethyl acetate=1:1 to 0:1) to afford tert-butyl 3-((4-(6-(cyclopropylmethoxy)-5-(pyrazolo[1,5-a]pyrimidine-3-carboxamido)-2H-indazol-2-yl)piperidin-1-yl)methyl)-3-fluoroazetidine-1-carboxylate (1.0 g, 1.62 mmol, 54% yield, >98% purity) as a yellow solid. MS (ESI) m/z: 619.3 [M+H]⁺. ¹H NMR (400 MHz, CDCl₃) δ=10.56 (s, 1H), 8.89 (s, 1H), 8.85-8.76 (m, 2H), 8.68 (dd, J=1.6, 4.0 Hz, 1H), 7.86 (s, 1H), 7.03 (dd, J=4.4, 7.2 Hz, 1H), 7.00 (s, 1H), 4.39-4.26 (m, 1H), 4.09-3.95 (m, 6H), 3.10 (br d, J=11.6 Hz, 2H), 2.88-2.78 (m, 2H), 2.49-2.37 (m, 2H), 2.27-2.18 (m, 4H), 1.55-1.43 (m, 10H), 0.75-0.69 (m, 2H), 0.56-0.50 (q, J=4.8 Hz, 2H).

### Step 2:

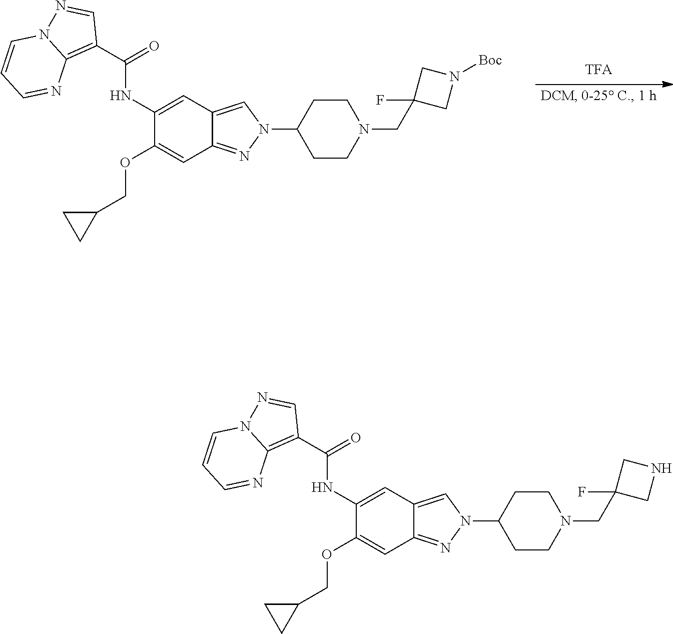 [SMILES: CC(C)(C)OC(=O)N1CC(F)(CN2CCC(n3cc4cc(NC(=O)c5cnn6cccnc56)c(OCC5CC5)cc4n3)CC2)C1 ; "DCM, 0-25° C., 1 h" ; O=C(Nc1cc2cn(C3CCN(CC4(F)CNC4)CC3)nc2cc1OCC1CC1)c1cnn2cccnc12 ; "TFA"]

To a mixture of tert-butyl 3-((4-(6-(cyclopropylmethoxy)-5-(pyrazolo[1,5-a]pyrimidine-3-carboxamido)-2H-indazol-2-yl)piperidin-1-yl)methyl)-3-fluoroazetidine-1-carboxylate (1.0 g, 1.62 mmol, 1 eq) in dichloromethane (20 mL) was added trifluoroacetic acid (11.85 g, 103.9 mmol, 7.69 mL, 64.3 eq) at 0° C. The reaction mixture was allowed to stir at 25° C. for 1 h under an atmosphere of nitrogen. The reaction mixture was concentrated under reduced pressure to give a residue which was redissolved in a 10:1 mixture of dichloromethane to methanol (200 mL) and ammonia hydrate (2 mL). The organic layer was separated, dried over anhydrous sodium sulfate, filtered, and concentrated under reduced pressure to give a residue. The crude product N-(6-(cyclopropylmethoxy)-2-(1-((3-fluoroazetidin-3-yl)methyl)piperidin-4-yl)-2H-indazol-5-yl)pyrazolo[1,5-a]pyrimidine-3-carboxamide (0.8 g, 1.54 mmol, 95% yield) was obtained as a yellow solid which was used in subsequent transformations without further purification. MS (ESI) m/z: 519.3 [M+H]⁺.

### Step 3:

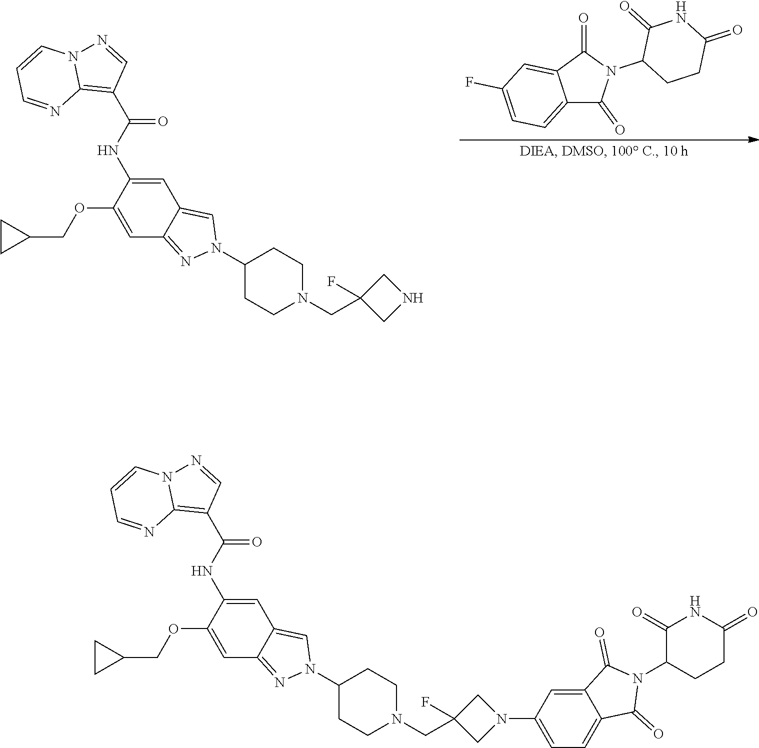

To a solution of N-(6-(cyclopropylmethoxy)-2-(1-((3-fluoroazetidin-3-yl)methyl)piperidin-4-yl)-2H-indazol-5-yl)pyrazolo[1,5-a]pyrimidine-3-carboxamide (0.8 g, 1.54 mmol, 1 eq) and 2-(2,6-dioxo-3-piperidyl)-5-fluoro-isoindoline-1,3-dione (420 mg, 1.52 mmol, 0.986 eq) in dimethyl sulfoxide (10 mL) was added diisopropylethylamine (1.00 g, 7.74 mmol, 1.35 mL, 5.02 eq) under an atmosphere of nitrogen. The mixture was stirred at 100° C. for 10 h. The reaction mixture cooled to room temperature and was poured into water (100 mL) at 0° C. The mixture was then extracted with dichloromethane (200 mL×3) and the combined organic layers were washed with brine (200 mL), dried over anhydrous sodium sulfate, concentrated in vacuo to give a residue. The residue was purified by silica gel column chromatography (eluted with dichloromethane:methanol=40:1 to 10:1) to afford N-(6-(cyclopropylmethoxy)-2-(1-((1-(2-(2,6-dioxopiperidin-3-yl)-1,3-dioxoisoindolin-5-yl)-3-fluoroazetidin-3-yl)methyl)piperidin-4-yl)-2H-indazol-5-yl)pyrazolo[1,5-a]pyrimidine-3-carboxamide (576.5 mg, 706.9 μmol, 46% yield, 95% purity) as a yellow solid. MS (ESI) m/z: 775.3 [M+H]⁺. ¹H NMR (400 MHz, DMSO-d₆) δ=11.14 (br s, 1H), 10.71 (s, 1H), 9.44 (dd, J=1.6, 7.2 Hz, 1H), 8.93 (dd, J=1.6, 4.4 Hz, 1H), 8.83-8.79 (m, 1H), 8.79-8.76 (m, 1H), 8.37 (s, 1H), 7.75 (d, J=8.0 Hz, 1H), 7.41-7.36 (m, 1H), 7.10 (s, 1H), 6.98 (d, J=2.0 Hz, 1H), 6.84 (dd, J=2.0, 8.4 Hz, 1H), 5.18-5.09 (m, 1H), 4.49-4.39 (m, 1H), 4.36-4.17 (m, 4H), 4.09 (d, J=6.8 Hz, 2H), 3.12 (br d, J=12.0 Hz, 2H), 3.05 (s, 1H), 2.99 (s, 1H), 2.97-2.88 (m, 1H), 2.69-2.61 (m, 2H), 2.52-2.44 (m, 2H), 2.24-2.04 (m, 5H), 1.62-1.51 (m, 1H), 0.81-0.74 (m, 2H), 0.52 (q, J=4.7 Hz, 2H).

TABLE 4
The following compounds may be prepared in an analogous fashion as described for the
synthesis of Example 161.

| | | ¹H NMR |
|---|---|---|
| | | All spectra acquired at 400 |
| Example | MS | MHz unless otherwise indicated |
| Example 161 | See Above | |
| 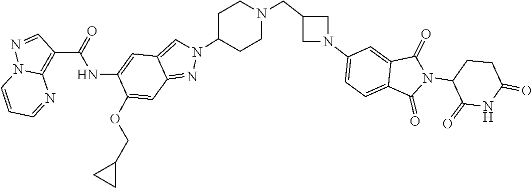 [SMILES: O=C1CCC(N2C(=O)c3ccc(N4CC(CN5CCC(n6cc7cc(NC(=O)c8cnn9cccnc89)c(OCC8CC8)cc7n6)CC5)C4)cc3C2=O)C(=O)N1] | 757.3 | CDCl₃ δ = 10.58 (s, 1H),8.91 (s, 1H), 8.83 (dd, J = 1.6, 6.8 Hz, 1H), 8.81 (s, 1H), 8.69 (dd, J = 1.6, 4.0 Hz, 1H), 8.05 (s, 1H), 7.87 (s, 1H), 7.66 (d, J = 8.0 Hz, 1H), 7.07-7.03 (m, 1H), 7.01 (s, 1H), 6.79 (d, J = 2.0 Hz, 1H), 6.52 (dd, J = 2.4, 8.4 Hz, 1H), 4.94 (dd, J = 5.2, 12.4Hz, 1H), 4.43-4.35 (m, 1H), 4.18 (t, J = 8.4 Hz, 2H), 4.03 (d, J = 6.8 Hz, 2H), 3.78-3.72 (m, 2H), 3.11-3.05 (m, 2H), 2.94-2.70 (m, 5H), 2.37-2.10 (m, 7H), 1.62-1.44 (m, 2H), 0.76-0.70 (m, 2H), 0.54 (q, J = 4.8 Hz, 2H) |
| Example 162 | | |
| 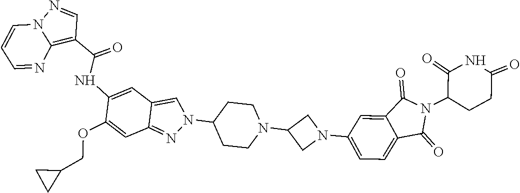 [SMILES: O=C1CCC(N2C(=O)c3ccc(N4CC(N5CCC(n6cc7cc(NC(=O)c8cnn9cccnc89)c(OCC8CC8)cc7n6)CC5)C4)cc3C2=O)C(=O)N1] | 743.3 | DMSO-d₆ δ = 11.08 (s, 1 H), 10.66 (s, 1 H), 9.43-9.26 (m, 2 H), 8.87 (dd, J = 1.6, 4.2 Hz, 1 H), 8.76 (s, 1 H), 8.71 (s, 1 H), 8.28 (s, 1 H), 7.68 (d, J = 8.2 Hz, 1 H), 7.36-7.29 (m, 1 H), 7.03 (s, 1 H), 6.81 (d, J = 1.6 Hz, 1 H), 6.67 (dd, J = 2.0, 8.4 Hz, 1 H), 5.06 (dd, J = 5.6, 12.8 Hz, 1H), 5.00-4.78 (m, 1 H), 4.73-4.60 (m, 2H), 4.23 (br t, J = 8.4 Hz, 2 H), 4.07-3.98 (m, 2 H), 3.83 (br t, J = 6.8 Hz, 2 H), 3.52 (br d, J = 10.4 Hz, 2H), 3.28 (br d, J = 6.8 Hz, 3 H), 3.02-2.84 (m, 3 H), 2.63-2.53 (m, 2 |
| Example 163 | | H), 2.06-1.96 (m, 3 H), 1.87 (br s, 1 |
| | | H), 1.75-1.57 (m, 2 H), 1.49 (br d, J = |
| | | 7.6 Hz, 1 H), 0.77-0.66 |
| | | (m, 2 H), 0.46 (q, J = 4.4 Hz, 2 H) |
| 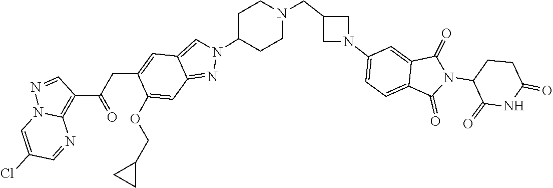 [SMILES: O=C1CCC(N2C(=O)c3ccc(N4CC(CN5CCC(n6cc7cc(CC(=O)c8cnn9cc(Cl)cnc89)c(OCC8CC8)cc7n6)CC5)C4)cc3C2=O)C(=O)N1] | 791.3 | DMSO-d₆ δ = 11.06 (s, 1 H), 10.43 (s, 1 H), 9.83 (d, J = 2.4 Hz, 1 H), 8.88 (d, J = 2.4 Hz, 1H), 8.73 (d, J = 3.2 Hz, 2H), 8.31 (s, 1 H), 8.13 (s, 1 H), 7.64 (d, J = 8.0 Hz, 1 H), 7.05 (s, 1 H), 6.78 (s, 1 H), 6.66 (dd, J = 8.4, 2.0 Hz, 1 H), 5.05 (dd, J = 13.2, 5.2 Hz, 1H), 4.39 (s, 1H), 4.16 (t, J = 8.0 Hz, 2 H), 4.03 (d, J = 7.2 Hz, 2 H), 3.73 (s, 2 H), 2.83-3.10 (m, 4 H), 2.66-2.79 (m, 2 H), 2.53-2.62 (m, 2 H), 2.21 (s, 2H), 1.96-2.14 (m, 5 H), 1.52 (s, 1 H), 0.65- 0.73 (m, 2 H), 0.39-0.47 (m, 2 H). |
| Example 164 | | |
| 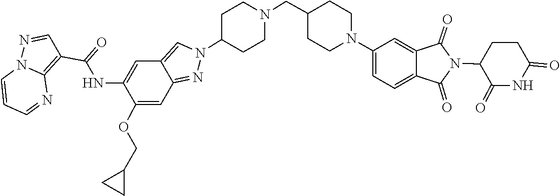 [SMILES: O=C1CCC(N2C(=O)c3ccc(N4CCC(CN5CCC(n6cc7cc(NC(=O)c8cnn9cccnc89)c(OCC8CC8)cc7n6)CC5)CC4)cc3C2=O)C(=O)N1] | 785.3 | DMSO-d₆ δ = 11.08 (s, 1 H), 10.64 (s, 1 H), 9.35 (dd, J = 1.2, 6.8 Hz, 1 H), 8.85 (dd, J = 1.6, 4.0 Hz, 1 H), 8.72 (d, J = 13.6 Hz, 2 H), 8.29 (s, 1 H), 7.64 (d, J = 8.4 Hz, 1 H), 7.34-7.27 (m, 2H), 7.21 (dd, J = 2.0, 8.8 Hz, 1 H), 7.03 (s, 1 H), 5.06 (dd, J = 5.2, 12.4 Hz, 1H), 4.34 (m, 1H), 4.12-3.89 (m, 4 H), 3.04-2.84 (m, 5 H), 2.64-2.53 (m, 2 H), 2.19 (d, J = 6.4 Hz, 2 H), 2.01 (m, 7 H), 1.81 (m, 3 H), 1.58-1.41 (m, 1 H), 1.22-1.09 (m, 2H), 0.78-0.63 (m, 2 H), 0.45 (m, 2 H) |
| Example 165 | | |
| 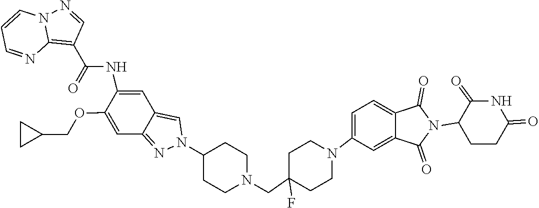 [SMILES: O=C1CCC(N2C(=O)c3ccc(N4CCC(F)(CN5CCC(n6cc7cc(NC(=O)c8cnn9cccnc89)c(OCC8CC8)cc7n6)CC5)CC4)cc3C2=O)C(=O)N1] | 803.3 | DMSO-d₆ δ = 11.07 (s, 1H), 10.64 (s, 1H), 9.38 (d, J = 6.8 Hz, 1H), 8.87 (d, J = 4.0 Hz, 1H), 8.73 (d, J = 10.0 Hz, 1H), 8.29 (s, 1H), 7.69 (d, J = 8.4 Hz, 1H), 7.39 (s, 1H), 7.33-7.29 (m, 2H), 7.03 (s, 1H), 5.09-5.05 (m, 1H), 4.36 (s, 2H), 4.02 (d, J = 5.6 Hz, 1H), 3.91 (s, 2H), 3.05 (s, 2H), 2.95-2.85 (m, 1H), 2.58-2.50 (m, 5H), 2.33-2.32 (m, 3H), 2.05-2.03 (m, 7H), 1.72 (s, 2H), 1.49 (s, 1H), 0.71 (d, J = 8.8 Hz, 2H), 0.47 (s, 2H). |
| Example 166 | | |
| 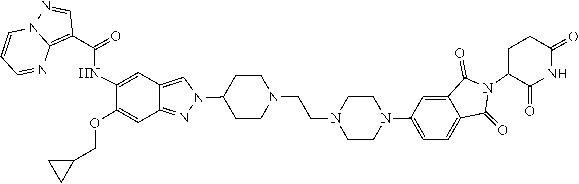 [SMILES: O=C1CCC(N2C(=O)c3ccc(N4CCN(CCN5CCC(n6cc7cc(NC(=O)c8cnn9cccnc89)c(OCC8CC8)cc7n6)CC5)CC4)cc3C2=O)C(=O)N1] | 800.3 | DMSO-d₆ δ = 11.36 (br s, 1 H) 11.10 (s, 1 H) 10.91 (br s, 1 H) 10.66 (s, 1 H) 9.38 (dd, J = 7.0, 1.6 Hz, 1 H) 8.87 (dd, J = 4.2, 1.6 Hz, 1 H) 8.77 (s, 1 H) 8.71 (s, 1 H) 8.33 (br s, 1 H) 7.77 (d, J = 8.4 Hz, 1H) 7.53 (s, 1 H) 7.37-7.45 (m, 1 H) 7.29-7.35 (m, 1 H) 7.10 (s, 1 H) 5.10 (dd, J = 12.8, 5.4 Hz, 1 H) 4.76 (br s, 1 H) 4.31 (br s, 1 H) 4.00 (br d, J = 12.4 Hz, 12 H) 3.42 (br d, J = 16.8 Hz, 1H) 3.27 (br s, 4 H) 2.84-2.95 (m, 1 H) 2.54-2.69 (m, 4 H) 2.42 |
| Example 167 | | (br s, 2H) 1.99- |
| | | 2.08 (m, 1 H) 1.45-1.55 (m, 1H) 0.67- |
| | | 0.74 (m, 2 H) 0.45 (q, J = 4.6 Hz, 2 H) |
| 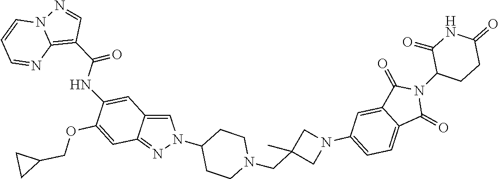 [SMILES: CC1(CN2CCC(n3cc4cc(NC(=O)c5cnn6cccnc56)c(OCC5CC5)cc4n3)CC2)CN(c2ccc3c(c2)C(=O)N(C2CCC(=O)NC2=O)C3=O)C1] | 771.2 | DMSO-d₆ δ = 11.08 (s, 1 H), 10.67 (s, 1 H), 9.38 (d, J = 7.2 Hz, 2 H), 8.87 (br d, J = 2.8 Hz, 1 H), 8.78 (s, 1 H), 8.71 (s, 1 H), 8.32 (s, 1 H), 7.69 (d, J = 8.4 Hz, 1 H), 7.33 (dd, J = 4.0, 7.0 Hz, 1H), 7.03 (s, 1H), 6.84 (s, 1H), 6.71 (br d, J = 9.8 Hz, 1 H), 5.13-5.00 (m, 1 H), 4.72 (br s, 1 H), 4.08-3.84 (m, 7 H), 3.54 (br s, 3 H), 2.86 (br d, J = 13.2 Hz, 1 H), 2.65-2.53 (m, 6 H), 2.13-1.94 (m, 2H), 1.61-1.42 (m, 4 H), 1.25-1.08 (m, 1H), 0.72 (br d, J = 8.2 |
| Example 168 | | Hz, 2 H), 0.46 (br d, J = 4.4 Hz, 2 H) |
| 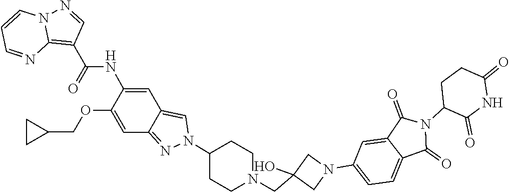 [SMILES: O=C1CCC(N2C(=O)c3ccc(N4CC(O)(CN5CCC(n6cc7cc(NC(=O)c8cnn9cccnc89)c(OCC8CC8)cc7n6)CC5)C4)cc3C2=O)C(=O)N1] | 773.1 | DMSO-d₆ δ = 11.09 (s, 1H), 10.65 (s, 1H), 9.37 (dd, J = 1.6, 7.2 Hz, 1H), 8.86 (dd, J = 1.6, 4.0 Hz, 1H), 8.77-8.67 (m, 2H), 8.28 (s, 1H), 7.64 (d, J = 8.4 Hz, 1H), 7.38-7.22 (m, 1H),7.03 (s, 1H),6.83 (d, J = 1.6 Hz, 1H), 6.69 (dd, J = 2.0, 8.4 Hz, 1H), 5.06 (dd, J = 5.2, 12.8 Hz, 1H), 4.44-4.29 (m, 1H), 4.13-3.99 (m, 4H), 3.91-3.79 (m, 2H), 3.13 (d, J = 11.2 Hz, 2H), 2.95-2.81 (m, 1H), 2.69 (s, 2H), 2.62-2.53 (m, 2H), 2.39-2.29 (m, 2H), 2.18-1.96 (m, 5H), 1.57-1.41 (m, 1H), 0.80-0.65 (m, 2H), 0.51-0.35 (m, 2H) |
| Example 169 | | |
| 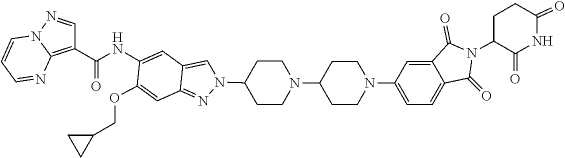 [SMILES: O=C1CCC(N2C(=O)c3ccc(N4CCC(N5CCC(n6cc7cc(NC(=O)c8cnn9cccnc89)c(OCC8CC8)cc7n6)CC5)CC4)cc3C2=O)C(=O)N1] | 771.3 | DMSO-d6 δ = 11.09 (s, 1H), 10.65 (s, 1 H), 9.36 (dd, J = 1.6, 6.8 Hz, 1 H), 8.86 (dd, J = 1.6, 4.0 Hz, 1 H), 8.72 (d, J = 12.0 Hz, 2 H), 8.29 (s, 1 H), 7.67 (d, J = 8.4 Hz, 1 H), 7.41-7.19 (m, 3 H), 7.03 (s, 1 H), 5.07 (dd, J = 4.8, 12.4 Hz, 1H), 4.50-4.34 (m, 1 H), 4.13 (m, 2 H), 4.01 (m, 2 H), 3.18-3.07 (m, 3 H), 2.99 (m, 3 H), 2.93-2.82 (m, 2 H), 2.60 (m, 2 H), 2.20- 2.06 (m, 4 H), 2.05-2.00 (m, 1 H), |
| Example 170 | | 1.96-1.82 (m, 2 H), 1.63-1.44 (m, 3 |
| | | H), 0.70 (m, 2 H), 0.52-0.36 (m, 2 H) |
| 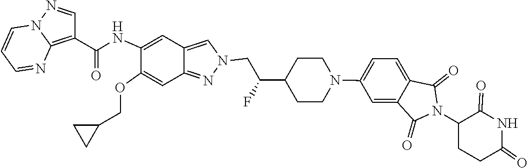 [SMILES: O=C1CCC(N2C(=O)c3ccc(N4CCC([C@H](F)Cn5cc6cc(NC(=O)c7cnn8cccnc78)c(OCC7CC7)cc6n5)CC4)cc3C2=O)C(=O)N1] | 734.3 | DMSO-d₆ δ = 11.07 (s, 1 H) 10.65 (s, 1 H) 9.37 (dd, J = 7.0, 1.6 Hz, 1 H) 8.87 (dd, J = 4.2, 1.6 Hz, 1 H) 8.75 (s, 1 H) 8.71 (s, 1 H) 8.27 (s, 1 H) 7.66 (d, J = 8.6 Hz, 1 H) 7.35 (d, J = 1.8 Hz, 1 H) 7.32 (dd, J = 7.0, 4.2 Hz, 1 H) 7.24-7.29 (m, 1 H) 7.04 (s, 1 H) 5.07 (dd, J = 12.8, 5.4 Hz, 1 H) 4.75-4.94 (m, 1 H) 4.59-4.74 (m, 2H) 4.14 (br d, J = 11.8 Hz, 2 H) 4.03 (br d, J = 7.6 Hz, 2 H) 2.83-3.00 (m, 3 H) 2.55-2.69 (m,2H) 1.83-2.05 (m, 4 H) 1.40-1.54 (m, 3 H) 0.68-0.74 |
| Example 171 | | (m, 2 H) 0.46 (q, J = 4.6 Hz, 2 H). |
| 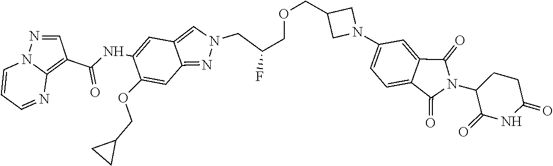 [SMILES: O=C1CCC(N2C(=O)c3ccc(N4CC(COC[C@H](F)Cn5cc6cc(NC(=O)c7cnn8cccnc78)c(OCC7CC7)cc6n5)C4)cc3C2=O)C(=O)N1] | 750.3 | DMSO-d₆ δ = 11.06 (s, 1H), 10.66 (s, 1H), 9.38 (dd, J = 1.6, 7.2 Hz, 1H), 8.87 (dd, J = 1.6, 4.0 Hz, 1H), 8.75 (s, 1H), 8.72 (s, 1H), 8.26 (s, 1H), 7.64 (d, J = 8.0 Hz, 1H), 7.32 (dd, J = 4.4, 7.2 Hz, 1H), 7.03 (s, 1H), 6.79 (d, J = 2.0 Hz, 1H), 6.65 (dd, J = 2.0, 8.0 Hz, 1H), 5.18 (s, 1H), 5.08-5.01 (m, 1H), 4.67 (m, 1H), 4.61 (d, J = 5.2 Hz, 1H), 4.11 (t, J = 8.8 Hz, 2H), 4.02 (d, J = 6.8 Hz, 2H), 3.84-3.77 (m, 3H), 3.76-3.64 (m, 4H), 3.09-2.96 (m, 1H), 2.94-2.79 (m, 1H), 2.64-2.56 |
| Example 172 | | (m, 1H), 2.04-1.96 (m, 1H), 1.54-1.44 |
| | | (m, 1H), 0.75-0.67 (m, 2H), 0.45 (m, 2H) |
| 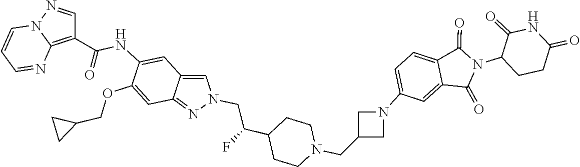 [SMILES: O=C1CCC(N2C(=O)c3ccc(N4CC(CN5CCC([C@H](F)Cn6cc7cc(NC(=O)c8cnn9cccnc89)c(OCC8CC8)cc7n6)CC5)C4)cc3C2=O)C(=O)N1] | 803.3 | DMSO-d₆ δ = 11.08 (s, 1 H), 10.66 (s, 1 H), 9.43-9.26 (m, 2 H), 8.87 (dd, J = 1.6, 4.2 Hz, 1 H), 8.76 (s, 1 H), 8.71 (s, 1 H), 8.28 (s, 1 H), 7.68 (d, J = 8.2 Hz, 1 H), 7.36-7.29 (m, 1 H), 7.03 (s, 1 H), 6.81 (d, J = 1.6 Hz, 1 H), 6.67 (dd, J = 2.0, 8.4 Hz, 1 H), 5.06 (dd, J = 5.6, 12.8 Hz, 1H), 5.00-4.78 (m, 1 H), 4.73-4.60 (m, 2H), 4.23 (br t, J = 8.4 Hz, 2 H), 4.07-3.98 (m, 2 H), 3.83 (br t, J = 6.8 Hz, 2 H), 3.52 (br d, J = 10.4 Hz, 2H), 3.28 (br d, J = 6.8 Hz, 3 H), |
| Example 173 | | 3.02-2.84 (m, 3 H), 2.63-2.53 (m, 2 |
| | | H), 2.06-1.96 (m, 3 H), 1.87 (br s, 1 |
| | | H), 1.75-1.57 (m, 2 H), 1.49 (br d, |
| | | J = 7.6 Hz, 1 H), 0.77-0.66 |
| | | (m, 2 H), 0.46 (q, J = 4.4 Hz, 2 H) |
| 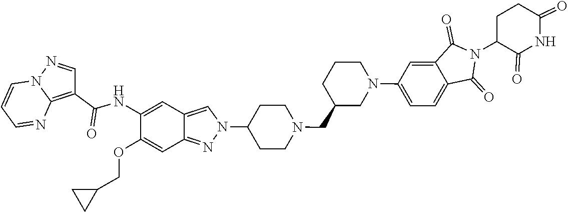 [SMILES: O=C1CCC(N2C(=O)c3ccc(N4CCC[C@H](CN5CCC(n6cc7cc(NC(=O)c8cnn9cccnc89)c(OCC8CC8)cc7n6)CC5)C4)cc3C2=O)C(=O)N1] | 785.2 | DMSO-d₆ δ = 11.06 (s, 1 H) 10.65 (s, 1 H) 9.38 (d, J = 6.8 Hz, 1 H) 8.87 (d, J = 4.0 Hz, 1 H) 8.73 (d, J = 13.6 Hz, 2 H) 8.32 (s, 1 H) 7.67 (d, J = 8.4 Hz, 1 H) 7.26-7.35 (m, 2 H) 7.22 (br d, J = 9.2 Hz, 1 H) 7.05 (s, 1 H) 5.05 (dd, J = 12.8, 5.2 Hz, 1H) 4.38 (br s, 1 H) 4.02 (d, J = 6.8 Hz, 2H) 3.80-3.96 (m, 2 H) 3.13 (m, 1 H) 3.05 (m 1 H) 2.83-2.99 (m, 3 H) 2.33 (br s, 1 H) 2.06-2.21 (m, 7 H) 2.00 (m 2 H) 1.84 (m2 H) 1.69 (br s, 1H) 1.50 (br s, 2H) 0.84 (m 1 H) 0.71 (br d, J = 8.0 Hz, 2 H) 0.46 (br d, J = 5.2 Hz, 2 H) |
| Example 174 | | |
| 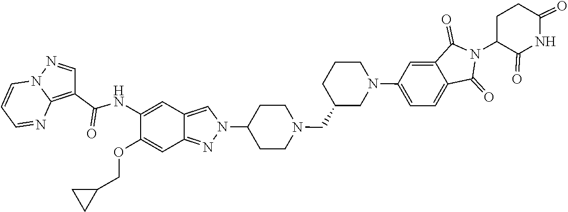 [SMILES: O=C1CCC(N2C(=O)c3ccc(N4CCC[C@@H](CN5CCC(n6cc7cc(NC(=O)c8cnn9cccnc89)c(OCC8CC8)cc7n6)CC5)C4)cc3C2=O)C(=O)N1] | 785.3 | DMSO-d₆ δ = 11.05 (s, 1H), 10.65 (s, 1H), 9.46-9.35 (m, 1H), 8.94-8.83 (m, 1H), 8.73 (d, J = 13.6 Hz, 2H), 8.32 (s, 1H), 7.67 (d, J = 8.8 Hz, 1H), 7.41-7.26 (m, 2H), 7.25-7.18 (m, 1H), 7.05 (s, 1H), 5.05 (dd, J = 5.6, 13.2 Hz, 1H), 4.50-4.28 (m, 1H), 4.02 (d, J = 7.2 Hz, 2H), 3.94-3.79 (m, 2H), 3.19-3.06 (m, 2H), 3.05-2.80 (m, 5H), 2.37-2.26 (m, 2H), 2.22-2.04 (m, 7H), 1.94-1.78 (m, 2H), 1.74-1.65 (m, 1H), 1.57-1.43 (m, 2H), 1.34-1.19 (m, 1H), 0.75-0.64 (m, 2H), 0.51-0.39 (m, 2H) |
| Example 175 | | |
| 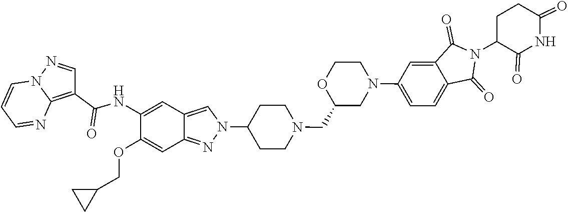 [SMILES: O=C1CCC(N2C(=O)c3ccc(N4CCO[C@@H](CN5CCC(n6cc7cc(NC(=O)c8cnn9cccnc89)c(OCC8CC8)cc7n6)CC5)C4)cc3C2=O)C(=O)N1] | 787.3 | DMSO-d₆ δ = 11.15-11.03 (m, 1H), 10.65 (s, 1H), 9.37 (dd, J = 1.6, 7.2 Hz, 1H), 8.87 (dd, J = 1.6, 4.0 Hz, 1H), 8.74 (s, 1H), 8.71 (s, 1H), 8.36 (s, 1H), 8.31 (s, 1H), 7.72 (d, J = 8.4 Hz, 1H), 7.38 (d, J = 2.0 Hz, 1H), 7.34-7.27 (m, 2H), 7.04 (s, 1H), 5.08 (dd, J = 4.8, 12.8 Hz, 1H), 4.41-4.32 (m, 1H), 4.02 (d, J = 6.8 Hz, 2H), 4.00-3.91 (m, 2H), 3.86 (d, J = 12.0 Hz, 1H), 3.79-3.72 (m, 1H), 3.68-3.59 (m, 2H), 3.13-3.01 (m, 3H), 2.97-2.85 (m, 2H), 2.75-2.68 (m, 1H), 2.63-2.57 (m, |
| Example 176 | | 1H), 2.31-2.19 (m, 2H), 2.18- |
| | | 1.98 (m, 6H), 1.55-1.45 (m, 1H), 0.74- |
| | | 0.67 (m, 2H), 0.48-0.43 (m, 2H) |
| 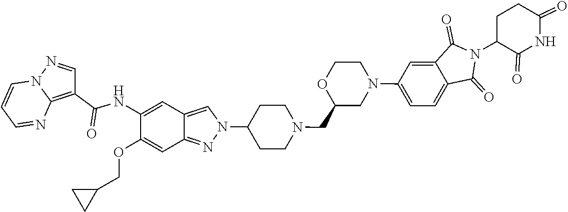 [SMILES: O=C1CCC(N2C(=O)c3ccc(N4CCO[C@H](CN5CCC(n6cc7cc(NC(=O)c8cnn9cccnc89)c(OCC8CC8)cc7n6)CC5)C4)cc3C2=O)C(=O)N1] | 787.3 | DMSO-d₆ δ = 11.09 (s, 1 H), 10.65 (s, 1 H), 9.38 (dd, J = 1.6, 7.2 Hz, 1 H), 8.87 (dd, J = 1.6, 4.0 Hz, 1 H), 8.73 (d, J = 11.2 Hz, 2 H), 8.31 (s, 1 H), 7.73 (d, J = 8.8 Hz, 1 H), 7.38 (d, J = 2.0 Hz, 1 H), 7.35-7.26 (m, 2H), 7.04 (s, 1 H), 5.08 (dd, J = 4.8, 12.8 Hz, 1H), 4.45-4.27 (m, 1 H), 4.02 (d, J = 6.8 Hz, 2 H), 3.96 (m, 2 H), 3.86 (m, 1 H), 3.76 (m, 1 H), 3.68-3.60 (m, 1 H), 3.16-2.98 (m, 3 H), 2.98-2.85 (m, 2 H), 2.73 (m, 1 H), 2.64-2.53 (m, 4 H), 2.30-2.18 (m, 2 H), 2.16-2.00 (m, 5 H), 1.56-1.42 (m, 1 H), 0.77-0.64 (m, 2 H), 0.46 (m, 2 H) |
| Example 177 | | |
| 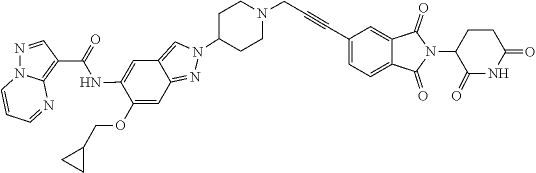 [SMILES: O=C1CCC(N2C(=O)c3ccc(C#CCN4CCC(n5cc6cc(NC(=O)c7cnn8cccnc78)c(OCC7CC7)cc6n5)CC4)cc3C2=O)C(=O)N1] | 726.2 | DMSO-d₆ δ = 11.14 (s, 1 H) 10.65 (s, 1 H) 9.37 (dd, J = 6.8, 1.6 Hz, 1 H) 8.86 (dd, J = 4.0, 1.6 Hz, 1 H) 8.74 (s, 1 H) 8.71 (s, 1 H) 8.31 (s, 1H) 7.90-7.99 (m, 3 H) 7.29-7.35 (m, 1 H) 7.05 (s, 1 H) 5.17 (dd, J = 12.8, 5.2 Hz, 1H) 4.38-4.50 (m, 1H) 4.02 (d, J = 7.2 Hz, 2 H) 3.69 (s, 2H) 3.03 (m, 2 H) 2.83-2.96 (m, 1 H) 2.51-2.70 (m, 4H) 2.03-2.18 (m, 5 H) 1.43-1.57 (m, 1 H) 0.67-0.75 (m, 2 H) 0.40-0.49 (m, 2 H). |
| Example 178 | | |
| 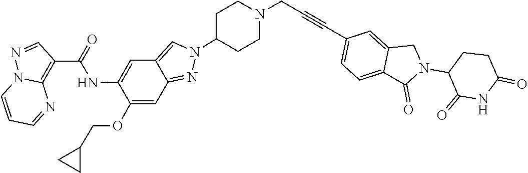 [SMILES: O=C1CCC(N2Cc3cc(C#CCN4CCC(n5cc6cc(NC(=O)c7cnn8cccnc78)c(OCC7CC7)cc6n5)CC4)ccc3C2=O)C(=O)N1] | 712.2 | DMSO-d₆ δ = 10.99 (br s, 1 H) 10.65 (s, 1 H) 9.38 (dd, J = 6.8, 1.6 Hz, 1 H) 8.87 (dd, J = 4.0, 1.6 Hz, 1 H) 8.75 (s, 1 H) 8.71 (s, 1 H) 8.24-8.37 (m, 1 H) 7.65-7.83 (m, 2 H) 7.59 (d,J = 8.4 Hz, 1 H) 7.32 (dd, J = 6.8, 4.0 Hz, 1 H) 7.06 (s, 1 H) 5.12 (dd, J = 13.6, 5.6 Hz, 1 H) 4.30-4.49 (m, 3H) 4.02 (d, J = 6.8 Hz, 2 H) 3.66 (s, 2 H) 3.02 (m, 2 H) 2.84-2.95 (m, 1 H) 2.57-2.69 (m, 2 H) 2.33-2.43 (m, 2 H) 2.15 (br s, 4 H) 1.96-2.06 (m, 1 H) 1.50 (br s, 1 H) 0.66-0.75 (m, 2H) 0.45 |
| Example 179 | | (m, 2 H). |
| 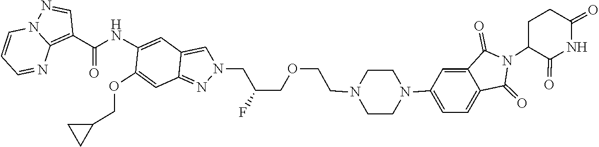 [SMILES: O=C1CCC(N2C(=O)c3ccc(N4CCN(CCOC[C@H](F)Cn5cc6cc(NC(=O)c7cnn8cccnc78)c(OCC7CC7)cc6n5)CC4)cc3C2=O)C(=O)N1] | 793.3 | CDCl₃ δ : 10.57 (s, 1 H) 8.90 (s, 1 H) 8.83 (dd, J = 7.0, 1.75 Hz, 1 H) 8.81 (s, 1 H) 8.69 (dd, J = 4.2, 1.8 Hz, 1 H) 8.14 (br d, J = 5.4 Hz, 1 H) 7.93 (s, 1 H) 7.68 (d, J = 8.6 Hz, 1 H) 7.30 (d, J = 2.0 Hz, 1 H) 7.02-7.10 (m, 2 H) 6.97 (s, 1 H) 5.02-5.27 (m, 1 H) 4.94 (dd, J = 11.0, 5.44 Hz, 1 H) 4.60-4.74 (m, 2 H) 4.01 (d, J = 6.8 Hz, 2 H) 3.53-3.81 (m, 4 H) 3.40-3.50 (m, 4 H) 2.66-2.90 (m, 9H) |
| Example 180 | | 2.07-2.21 (m, 1 H) 1.44-1.57 (m, 1 H) |
| | | 0.66-0.81 (m, 2 H) 0.46-0.61 (m, 2H) |
| 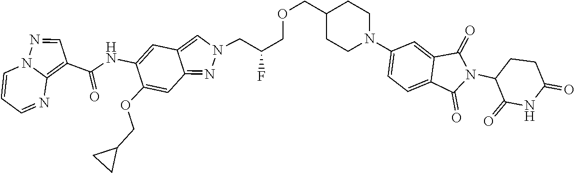 [SMILES: O=C1CCC(N2C(=O)c3ccc(N4CCC(COC[C@H](F)Cn5cc6cc(NC(=O)c7cnn8cccnc78)c(OCC7CC7)cc6n5)CC4)cc3C2=O)C(=O)N1] | 778.2 | DMSO-d₆ δ= 11.03-11.10 (m, 1 H) 10.62-10.69 (m, 1 H) 9.35-9.41 (m, 1 H) 8.84-8.89 (m, 1 H) 8.74-8.76 (m, 1 H) 8.71-8.73 (m, 1 H) 8.29 (s, 1 H) 7.62-7.67 (m, 1 H) 7.29-7.35 (m, 2 H) 7.21-7.26 (m, 1 H)7.01-7.07 (m, 1H) 5.24 (s, 2 H) 4.57-4.70 (m, 2 H) 4.00-4.10 (m, 4 H) 3.56-3.76 (m, 3 H) 3.30-3.35 (m, 2 H) 2.81-3.05 (m, 3 H) 2.54 (s, 1 H) 1.96-2.06 (m, 1 H) 1.84-1.94 (m, 1 H) 1.73-1.82 (m,2H)1.43-1.57 (m, 1 H) 1.17-1.32 (m, 2 H) 0.66-0.76 (m, 2 H) 0.41-0.51 |
| Example 181 | | (m, 2H) |
| 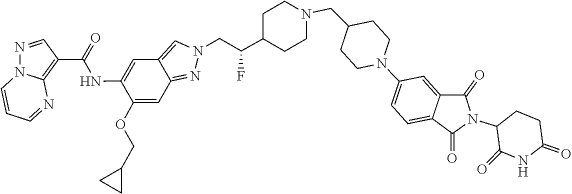 [SMILES: O=C1CCC(N2C(=O)c3ccc(N4CCC(CN5CCC([C@H](F)Cn6cc7cc(NC(=O)c8cnn9cccnc89)c(OCC8CC8)cc7n6)CC5)CC4)cc3C2=O)C(=O)N1] | 831.4 | CDCl₃ δ : 10.56 (s, 1 H) 8.89 (s, 1 H) 8.82 (dd, J = 7.0, 1.6 Hz, 1H)8.79 (s, 1 H) 8.67 (dd, J = 4.2, 1.7 Hz, 1 H) 8.24-8.63 (m, 1 H) 7.90 (s, 1 H) 7.66 (d, J = 8.6 Hz, 1 H) 7.27 (s, 1 H) 7.00-7.06 (m, 2 H) 6.98 (s, 1 H) 4.94 (dd, J = 12.4, 5.4 Hz, 1 H) 4.66-4.84 (m, 1 H) 4.43-4.65 (m, 2H) 4.01 (d, J = 6.7 Hz, 2 H) 3.94 (br d, J = 12.4 Hz, 2 H) 2.66-3.04 (m, 8 H) 2.16-2.23 (m, 2 H) 2.09-2.15 (m, 1 H) 1.82-1.97 (m, 8H) 1.44-1.66 (m, 4 H) 0.68-0.76 |
| Example 182 | | (m, 2 H) 0.53 (q, J = 4.89 Hz, 2 H) |
| 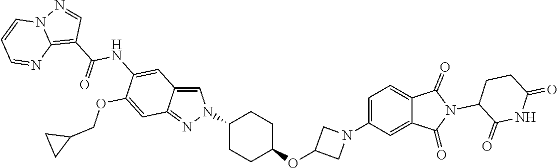 [SMILES: O=C1CCC(N2C(=O)c3ccc(N4CC(O[C@H]5CC[C@H](n6cc7cc(NC(=O)c8cnn9cccnc89)c(OCC8CC8)cc7n6)CC5)C4)cc3C2=O)C(=O)N1] | 758.2 | DMSO-d₆ δ = 11.07 (s, 1 H) 10.64 (s, 1 H) 9.36 (dd, J = 6.8, 1.6 Hz, 1 H) 8.86 (dd, J = 4.0, 1.6 Hz, 1 H) 8.74 (s, 1 H) 8.71 (s, 1 H) 8.26 (s, 1 H) 7.64 (d, J = 8.4 Hz, 1 H) 7.31 (dd, J = 6.8, 4.4 Hz, 1 H) 7.03 (s, 1 H) 6.80 (d, J = 1.6 Hz, 1H) 6.66 (dd, J = 8.4, 2.0 Hz, 1 H) 5.06 (dd, J = 12.4, 5.2 Hz, 1 H) 4.64 (br t, J = 4.4 Hz, 1 H) 4.40 (br t, J = 12.0 Hz, 1 H) 4.27-4.35 (m, 2 H) 4.02 (d, J = 6.8 Hz, 2 H) 3.85 (br dd, J = 9.2, 4.0 Hz, 2H) 3.50 (br t, J = |
| Example 183 | | 10.8 Hz, 1 H) 2.82-2.95 (m, 1 H) |
| | | 2.54-2.63 (m, 2 H) 2.11 (br t, J = 14.4 |
| | | Hz, 4 H) 2.02 (m, 1H) 1.87-1.98 (m, 2 |
| | | H) 1.49 (m, 3 H) 0.68-0.74 (m, 2H) |
| | | 0.43-0.49 (m, 2 H) |
| 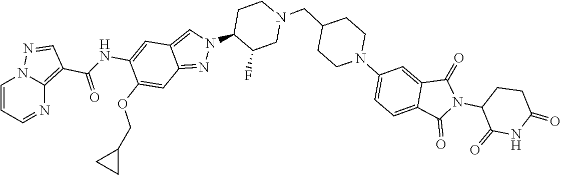 [SMILES: O=C1CCC(N2C(=O)c3ccc(N4CCC(CN5CC[C@H](n6cc7cc(NC(=O)c8cnn9cccnc89)c(OCC8CC8)cc7n6)[C@@H](F)C5)CC4)cc3C2=O)C(=O)N1] | 803.2 | DMSO-d₆ δ = 11.07 (br s, 1H), 10.66 (s, 1H), 9.38 (dd, J = 1.6, 6.8 Hz, 1H), 8.87 (dd, J = 1.6, 4.4 Hz, 1H), 8.75 (s, 1H), 8.72 (s, 1H),8.36(s, 1H), 7.66 (d, J = 8.4 Hz, 1H), 7.34-7.30 (m, 2H), 7.24 (dd, J = 2.0, 8.8 Hz, 1H), 7.06 (s, 1H), 5.15-4.91 (m, 2H), 4.55 (br dd, J = 4.0, 9.2 Hz, 1H), 4.08 (br s, 1H), 4.03 (br d, J = 6.8 Hz, 3H), 3.04-2.83 (m, 4H), 2.69-2.54 (m,2H), 2.34-2.29 (m, 2H), 2.23-2.13 (m, 3H), 2.13-1.97 |
| Example 184 | | (m, 3H), 1.91-1.78 (m, 3H), 1.55-1.47 |
| | | (m, 1H), 1.25-1.12 (m, 2H), 0.74-0.68 |
| | | (m, 2H), 0.46 (q, J = 5.2 Hz, 2H) |
| 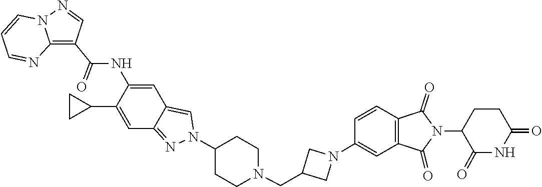 [SMILES: O=C1CCC(N2C(=O)c3ccc(N4CC(CN5CCC(n6cc7cc(NC(=O)c8cnn9cccnc89)c(C8CC8)cc7n6)CC5)C4)cc3C2=O)C(=O)N1] | 727.2 | DMSO-d₆ δ = 11.07 (s, 1 H), 10.34 (s, 1 H), 9.39 (dd, J = 6.8, 1.6 Hz, 1 H), 8.91 (dd, J = 4.0, 1.6 Hz, 1 H), 8.73 (s, 1 H), 8.65 (s, 1 H), 8.38 (s, 1 H), 8.27 (s, 1 H), 7.63 (d, J = 8.4 Hz, 1 H), 7.42 (s, 1 H), 7.29-7.36 (m, 1 H), 6.78 (s, 1 H), 6.65 (dd, J = 8.4, 2.0 Hz, 1 H), 5.05 (dd, J = 12.8, 5.2 Hz, 1 H), 4.42 (t, J = 7.2 Hz, 1 H), 4.15 (t, J = 8.0 Hz, 2 H), 3.69-3.73 (m, 2 H), 2.96-3.04 (m, 3 H), 2.86 (dd, J = 17.6, 5.6 Hz, 1 H), 2.67 (d, J = 7.2 Hz, 2 H), 2.52-2.57 (m, 2 H), 2.15-2.24 (m, 2 H), 2.09 (s, 5 H), 1.98-2.04 (m, 1 H), |
| Example 185 | | 1.15-1.20 (m, 2 H), 0.75-0.80 (m, 2 H). |
| 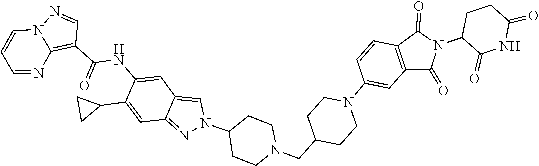 [SMILES: O=C1CCC(N2C(=O)c3ccc(N4CCC(CN5CCC(n6cc7cc(NC(=O)c8cnn9cccnc89)c(C8CC8)cc7n6)CC5)CC4)cc3C2=O)C(=O)N1] | 755.2 | DMSO-d₆ δ = 11.07 (s, 1 H) 10.34 (s, 1 H) 9.39 (d, J = 7.2 Hz, 1 H) 8.91 (d, J = 2.8 Hz, 1 H) 8.73 (s, 1 H) 8.64 (s, 1 H) 8.37 (s, 1 H) 8.18 (s, 1H) 7.65 (d, J = 8.4 Hz, 1 H)7.42 (s, 1 H)7.31-7.36 (m, 2 H) 7.24 (br d, J = 10.0 Hz, 1 H) 5.06 (dd, J = 12.8, 5.2 Hz, 1 H) 4.42 (br s, 1 H) 4.06 (br d, J = 11.38 Hz, 2 H) 2.94-3.02 (m, 4 H) 2.87 (br d, J = 11.6Hz, 1H) 2.58(m, 2 H) 2.22 (m, 2 H) 2.06-2.15 (m, 8 H) 1.98-2.04 (m, 1H) 1.83 (br d, J = 11.6 Hz, 2 H) |
| Example 186 | | 1.17 (br d, J = 9.2 Hz, 4 H) 0.78 (br d, |
| | | J = 4.0 Hz, 2 H). |
| 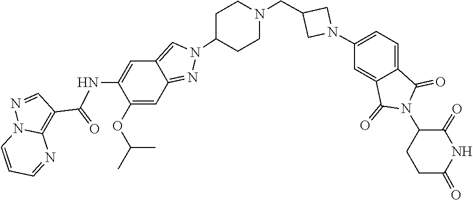 [SMILES: CC(C)Oc1cc2nn(C3CCN(CC4CN(c5ccc6c(c5)C(=O)N(C5CCC(=O)NC5=O)C6=O)C4)CC3)cc2cc1NC(=O)c1cnn2cccnc12] | 745.3 | DMSO-d₆ δ = 11.09 (s, 1H), 10.60 (s, 1H), 9.38 (d, J = 7.2 Hz, 1H), 8.91-8.83 (m, 1H), 8.73 (d, J = 19.6 Hz, 2H), 8.32 (s, 1H), 7.63 (d, J = 8.4 Hz, 1H), 7.34 (dd, J = 4.4, 7.2 Hz, 1H), 7.14 (s, 1H), 6.77 (br s, 1H), 6.64 (br d, J = 7.6 Hz, 1H), 5.06 (dd, J = 5.2, 12.8 Hz, 1H), 4.86 (td, J = 6.0, 12.0 Hz, 1H), 4.46-4.28 (m, 1H), 4.15 (br t, J = 8.4 Hz, 2H), 3.79-3.63 (m, 2H), 3.08-2.83 (m, 4H), 2.67 (br d, J = 6.4 Hz, 2H), 2.61 (br d, J = 2.4 Hz, 1H), 2.58-2.54 (m, 1H), 2.26-2.13 (m, 2H), 2.07 (br s, 5H), 1.48 (d, J = 5.6 Hz, 6H). |
| Example 187 | | |
| 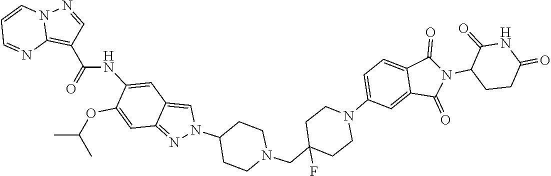 [SMILES: CC(C)Oc1cc2nn(C3CCN(CC4(F)CCN(c5ccc6c(c5)C(=O)N(C5CCC(=O)NC5=O)C6=O)CC4)CC3)cc2cc1NC(=O)c1cnn2cccnc12] | 791.3 | DMSO-d₆ δ = 11.08 (s, 1H), 10.59 (s, 1H), 9.39 (dd, J = 1.6, 7.2 Hz, 1H), 8.88 (dd, J = 1.6, 4.4 Hz, 1H), 8.73 (d, J = 14.4 Hz, 2H), 8.31 (s, 1H),7.69 (d, J = 8.8 Hz, 1H), 7.40 (s, 1H), 7.36-7.29 (m, 2H), 7.13 (s, 1H), 5.08 (dd, J = 5.6, 12.8 Hz, 1H), 4.85 (td, J = 6.0, 12.0 Hz, 1H), 4.41-4.31 (m, 1H), 3.90 (br d, J = 13.2 Hz, 2H), 3.27 (br s, 1H), 3.05 (br d, J = 11.2 Hz, 2H), 2.95- 2.84 (m, 1H), 2.66-2.58 (m, 4H), 2.38-2.32 (m, 2H), 2.15-1.92 (m, 8H), 1.86-1.70 |
| Example 188 | | (m, 2H), 1.48 (d, J = 5.6 Hz, 6H). |
| 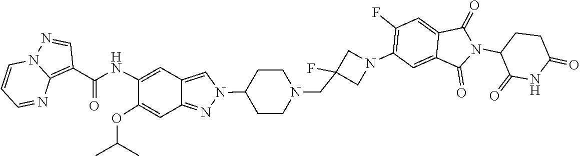 [SMILES: CC(C)Oc1cc2nn(C3CCN(CC4(F)CN(c5cc6c(cc5F)C(=O)N(C5CCC(=O)NC5=O)C6=O)C4)CC3)cc2cc1NC(=O)c1cnn2cccnc12] | 781.0 | DMSO-d₆ δ = 11.09 (s, 1H), 10.59 (s, 1H), 9.38 (d, J = 7.2 Hz, 1H), 8.87 (br d, J = 4.0 Hz, 1H), 8.73 (d, J = 15.2 Hz, 2H), 8.31 (s, 1H), 7.66 (d, J = 11.2 Hz, 1H), 7.34 (dd, J = 4.0, 6.4 Hz, 1H), 7.13 (s, 1H), 7.07 (d, J = 7.6 Hz, 1H), 5.08 (br dd, J = 4.8, 12.8 Hz, 1H), 4.85 (td, J = 5.6, 11.6 Hz, 1H), 4.42-4.25 (m, 5H), 4.10-3.97 (m, 1H), 3.29 (br s, 2H), 3.11-3.02 (m, 2H), 2.99 (s, 1H), 2.93 (s, 1H), 2.90-2.81 (m, 1H), 2.18- |
| Example 189 | | 2.06 (m, 4H), 1.99 (s, 1H), 1.48 (d, J = 6.0 |
| | | Hz, 6H), 1.17 (t, J = 7.2 Hz, 1H) |
| 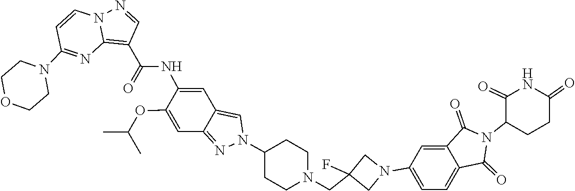 [SMILES: CC(C)Oc1cc2nn(C3CCN(CC4(F)CN(c5ccc6c(c5)C(=O)N(C5CCC(=O)NC5=O)C6=O)C4)CC3)cc2cc1NC(=O)c1cnn2ccc(N3CCOCC3)nc12] | 848.5 | DMSO-d₆ δ = 11.07 (s, 1H), 9.51 (s, 1H),8.85(d, J = 8.0 Hz, 1H), 8.30 (s, 3H), 7.69 (d, J = 8.4 Hz, 1H), 7.13 (s, 1H), 6.97-6.90 (m, 2H), 6.82-6.74 (m, 1H), 5.07 (dd, J = 5.6, 13.2 Hz, 1H), 4.70-4.59 (m, 1H), 4.47-4.33 (m, 1H), 4.29-4.11 (m, 4H), 3.89-3.69 (m, 9H), 3.29 (s, 1H), 3.06 (br d, J = 10.8 Hz, 2H), 2.99 (s, 1H), 2.93 (s, 1H), 2.54 (s, 2H), 2.21-1.96 (m, 6H), 1.30 (d, J = 6.0 Hz, 6H) |
| Example 190 | | |
| 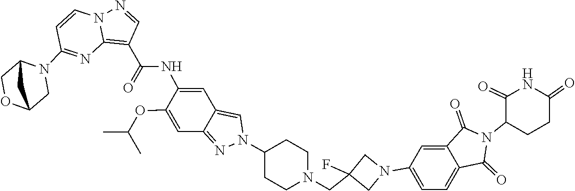 [SMILES: CC(C)Oc1cc2nn(C3CCN(CC4(F)CN(c5ccc6c(c5)C(=O)N(C5CCC(=O)NC5=O)C6=O)C4)CC3)cc2cc1NC(=O)c1cnn2ccc(N3C[C@H]4C[C@@H]3CO4)nc12] | 860.3 | DMSO-d₆ δ = 11.07 (br s, 1H), 9.78-9.64 (m, 1H), 8.80 (d, J = 7.8 Hz, 1H), 8.37-8.24 (m, 3H), 7.69 (d, J = 8.3Hz, 1H), 7.14(br d, J = 12.6 Hz, 1H), 6.92 (d, J = 2.0 Hz, 1H), 6.90-6.44 (m, 2H), 5.45-5.03 (m, 2H), 4.79 (br d, J = 8.2 Hz, 1H), 4.70-4.60 (m, 1H), 4.44-4.34 (m, 1H), 4.30-4.10 (m, 4H), 3.88- 3.70 (m, 2H), 3.64 (br d, J = 9.0 Hz, 1H), 3.06 (br d, J = 11.6 Hz, 2H), 3.01-2.91 (m, 2H), 2.59-2.53 (m, 3H), 2.46-2.37 (m, 3H), 2.19-1.96 (m, 7H), 1.42-1.31 (m, 3H), 1.28 (br t, J = 5.4 Hz, 3H) |
| Example 191 | | |
| 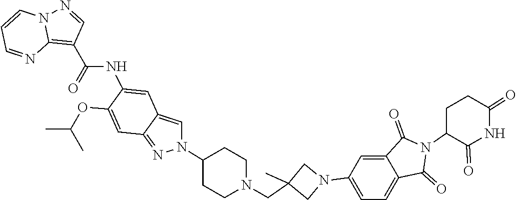 [SMILES: CC(C)Oc1cc2nn(C3CCN(CC4(C)CN(c5ccc6c(c5)C(=O)N(C5CCC(=O)NC5=O)C6=O)C4)CC3)cc2cc1NC(=O)c1cnn2cccnc12] | 759.2 | DMSO-d₆ δ = 11.06 (s, 1H), 10.75 (s, 1H), 9.40 (dd, J = 1.4, 7.0 Hz, 1H), 8.89 (dd, J = 1.6, 4.2 Hz, 1H), 8.73 (d, J = 2.6 Hz, 2H), 7.64 (d, J = 8.4 Hz, 1H), 7.36 (dd, J = 4.4, 7.0 Hz, 1H), 7.33 (s, 1H), 6.79 (d, J = 1.8 Hz, 1H), 6.66 (dd, J = 2.0, 8.4 Hz, 1H), 5.07 (dd, J = 5.4, 13.0 Hz, 1H), 4.89 (td, J = 5.8, 12.0 Hz, 1H), 4.40 (s, 2H), 4.04-3.93 (m, 1H), 3.82-3.73 (m, 4H), 2.95-2.79 (m, 3H), 2.57-2.53 (m, 3H), 2.19 (br t, J = 10.8 Hz, 2H), 2.01 (br dd, J = 5.0, 10.0 Hz, 1H), 1.85-1.72 (m, 2H), 1.71-1.62 (m, 2H), 1.44 (d, J = 6.0 Hz, 6H), 1.38 (s, |
| Example 192 | | 3H), 1.27-1.21 (m, 1H) |
| 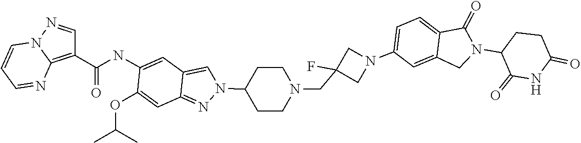 | 774.3 | DMSO-d₆ δ = 10.94 (s, 1H), 10.59 (s, 1H), 9.38 (dd, J = 1.2, 7.2 Hz, 1H), 8.87 (dd, J = 1.2, 4.4 Hz, 1H), 8.74 (s, 1H), 8.71 (s, 1H), 8.31 (s, 1H), 7.53 (d, J = 8.4 Hz, 1H), 7.34 (dd, J = 4.4, 6.8 Hz, 1H), 7.13 (s, 1H), 6.64 (s, 1H), 6.62-6.56 (m, 1H), 5.10-5.00 (m, 1H), 4.91-4.80 (m, 1H), 4.39-4.28 (m, 2H), 4.25-3.98 (m, 6H), 3.06 (br d, J = 10.4 |
| Example 193 | | Hz, 3H), 2.98 (s, 1H), 2.92 (s, 2H), |
| | | 2.42-2.37 (m, 2H), 2.14-2.03 (m, 4H), |
| | | 2.01-1.91 (m, 1H), 1.48 (s, 3H), 1.47 |
| | | (s, 3H) |
| 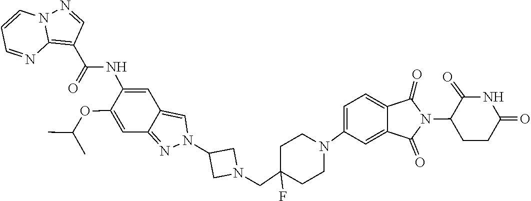 [SMILES: CC(C)Oc1cc2nn(C3CN(CC4(F)CCN(c5ccc6c(c5)C(=O)N(C5CCC(=O)NC5=O)C6=O)CC4)C3)cc2cc1NC(=O)c1cnn2cccnc12] | 763.3 | DMSO-d₆ δ = 11.08 (s, 1H), 10.60 (s, 1H), 9.38 (dd, J = 1.6, 7.2 Hz, 1H), 8.87 (dd, J = 1.6, 4.0 Hz, 1H), 8.73 (d, J = 16.0 Hz, 2H), 8.37 (s, 1H), 7.68 (d, J = 8.4 Hz, 1H), 7.38 (d, J = 2.0 Hz, 1H), 7.34 (dd, J = 4.4, 7.2 Hz, 1H), 7.29 (dd, J = 2.0, 8.8 Hz, 1H), 7.18 (s, 1H), 5.23 (quin, J = 6.8 Hz, 1H), 5.07 (dd, J = 5.6, 12.8 Hz, 1H), 4.87 (quin, J = 6.0 Hz, 1H), 3.92-3.83 (m, 4H), 3.65 (br t, J = 7.2 Hz, 2H), 3.30-3.23 (m, 2H), 2.94-2.84 (m, 1H), 2.83-2.75 (m, 2H), 2.63-2.57 (m, 1H), 2.06-1.86 (m, 4H), 1.84-1.67 (m, 2H), 1.48 (d, J = 6.0 Hz, 6H) |
| Example 194 | | |
| 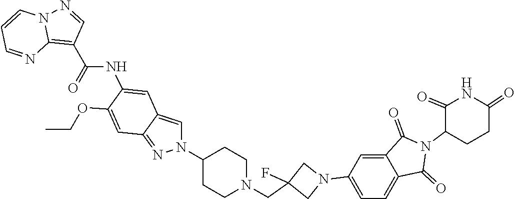 [SMILES: CCOc1cc2nn(C3CCN(CC4(F)CN(c5ccc6c(c5)C(=O)N(C5CCC(=O)NC5=O)C6=O)C4)CC3)cc2cc1NC(=O)c1cnn2cccnc12] | 748.6 | DMSO-d₆ δ = 11.07 (s, 1H), 10.64 (s, 1H), 9.38 (dd, J = 1.2, 7.2 Hz, 1H), 8.86 (dd, J = 1.2, 4.0 Hz, 1H), 8.72 (d, J = 4.8 Hz, 2H), 8.31 (s, 1H),7.69(d, J = 8.4 Hz, 1H), 7.34 (dd, J = 4.0, 6.8 Hz, 1H), 7.08 (s, 1H), 6.96-6.87 (m, 1H), 6.82-6.74 (m, 1H), 5.07 (dd, J = 5.6, 12.8 Hz, 1H), 4.45-4.33 (m, 1H), 4.31-4.11 (m, 6H), 3.29 (s, 1H), 3.06 (br d, J = 11.6 Hz, 2H), 3.01-2.90 (m, 2H), 2.90-2.83 (m, 1H), 2.64-2.53 (m, 3H), 2.20-1.93 (m, 5H), 1.61 (t, J = 6.8 Hz, 3H) |
| Example 195 | | |
| 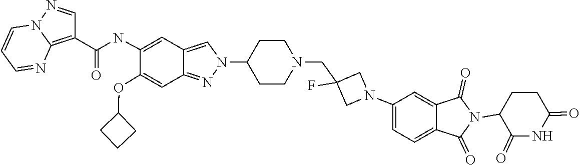 | 775.3 | DMSO-d₆ δ = 11.07 (s, 1H), 10.61 (s, 1H), 9.39 (d, J = 7.0 Hz, 1H), 8.88 (d, J = 4.0 Hz, 1H), 8.72 (d, J = 7.2 Hz, 1H), 8.32 (s, 1H), 8.19 (s, 1H), 7.63 (d, J = 8.4 Hz, 1H), 7.34 (dd, J = 4.4, 7.2 Hz, 1H), 6.91 (s, 1H), 6.78 (s, 1H), 6.65 (br d, J = 8.4 Hz, 1H), 5.06 (dd, J = 5.2, 12.8 Hz, 1H), 4.92 (t, J = 6.8 Hz, 1H), 4.37 (br t, J = 7.6 Hz, 1H), 4.15 (br t, J = 8.0 Hz, 2H), 3.74-3.70 (m, 2H), 2.98 (br d, J = 10.8 Hz, 2H), 2.90-2.83 (m, 1H), 2.67 (br d, J = 6.0 Hz, 2H), 2.65-2.61 |
| Example 196 | | (m, 2H), 2.61 (br s, 1H), 2.54 (s, 1H), |
| | | 2.33-2.25 (m, |
| | | 3H), 2.21-2.15 (m, 2H), 2.07 (br s, |
| | | 5H), 1.96-1.89 (m, 1H), 1.82-1.75 (m, 1H) |
| 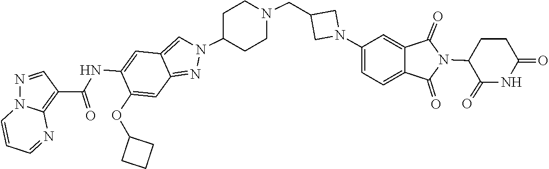 [SMILES: O=C1CCC(N2C(=O)c3ccc(N4CC(CN5CCC(n6cc7cc(NC(=O)c8cnn9cccnc89)c(OC8CCC8)cc7n6)CC5)C4)cc3C2=O)C(=O)N1] | 757.3 | DMSO-d₆ δ = 11.07 (s, 1H), 10.61 (s, 1H), 9.39 (d, J = 7.0 Hz, 1H), 8.88 (d, J = 4.0 Hz, 1H), 8.72 (d, J = 7.2 Hz, 1H), 8.32 (s, 1H), 8.19 (s, 1H), 7.63 (d, J = 8.4 Hz, 1H), 7.34 (dd, J = 4.4, 7.2 Hz, 1H), 6.91 (s, 1H), 6.78 (s, 1H), 6.65 (br d, J = 8.4 Hz, 1H), 5.06 (dd, J = 5.2, 12.8 Hz, 1H), 4.92 (1, J = 6.8 Hz, 1H), 4.37 (br t, J = 7.6 Hz, 1H), 4.15 (br 1,J = 8.0 Hz, 2H), 3.74-3.70 (m, 2H), 2.98 (br d, J = 10.8 Hz, 2H), 2.90-2.83 (m, 1H), 2.67 (br d,J = 6.0 Hz, 2H), 2.65-2.61 |
| Example 197 | | (m, 2H), 2.61 (br s, |
| | | 1H), 2.54 (s, 1H), 2.33-2.25 (m, 3H), |
| | | 2.21-2.15 (m, 2H), 2.07 (br s, 5H), |
| | | 1.96-1.89 (m, 1H), 1.82-1.75 (m, 1H) |
| 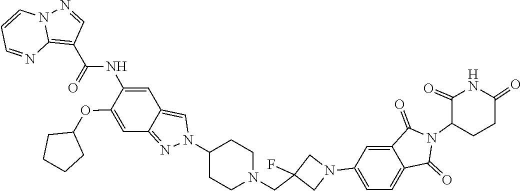 [SMILES: O=C1CCC(N2C(=O)c3ccc(N4CC(F)(CN5CCC(n6cc7cc(NC(=O)c8cnn9cccnc89)c(OC8CCCC8)cc7n6)CC5)C4)cc3C2=O)C(=O)N1] | 789.3 | DMSO-d₆ δ = 11.13-11.05 (m, 1H), 10.50-10.44 (m, 1H), 9.42-9.33 (m, 1H), 8.82-8.79 (m, 1H), 8.76-8.74 (m, 1H), 8.72-8.70 (m, 1H), 8.32-8.28 (m, 1H), 7.71-7.66 (m, 1H), 7.37-7.31 (m, 1H), 7.06-7.01 (m, 1H), 6.94-6.89 (m, 1H), 6.81-6.73 (m, 1H), 5.11-5.01 (m, 2H), 4.42-4.32 (m, 1H), 4.29-4.11 (m, 4H), 3.10-3.01 (m, 2H), 3.01-2.91 (m, 2H), 2.90-2.82 (m, 1H), 2.63-2.54 (m, 2H), 2.46-2.36 (m, 2H), 2.18-1.96 (m, 9H), 1.92-1.80 (m, 2H), 1.74-1.63 (m, 2H) |
| Example 198 | | |
| 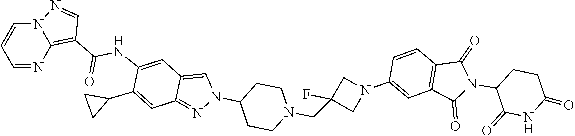 [SMILES: O=C1CCC(N2C(=O)c3ccc(N4CC(F)(CN5CCC(n6cc7cc(NC(=O)c8cnn9cccnc89)c(C8CC8)cc7n6)CC5)C4)cc3C2=O)C(=O)N1] | 745.3 | DMSO-d₆ δ = 11.08 (s, 1H), 10.34 (s, 1H), 9.43-9.35 (m, 1H), 8.91 (br d, J =2.8 Hz, 1H), 8.73 (s, 1H), 8.64 (s, 1H), 8.37 (s, 1H), 7.69 (d, J = 8.4 Hz, 1H), 7.42 (s, 1H), 7.33 (dd, J = 4.2, 6.8 Hz, 1H), 6.92 (s, 1H), 6.82-6.72 (m, 1H), 5.07 (br dd, J = 5.4, 13.0 Hz, 1H),4.44 (br d, J = 4.2 Hz, 1H), 4.31-4.10 (m, 4H), 3.14-2.80 (m, 6H), 2.45-2.38 (m, 2H), |
| Example 199 | | 2.18-1.97 (m, 7H), 1.17 |
| | | (br d, J = 7.8 Hz, 2H), 0.78 (br d, J = |
| | | 4.4 Hz, 2H) |
| 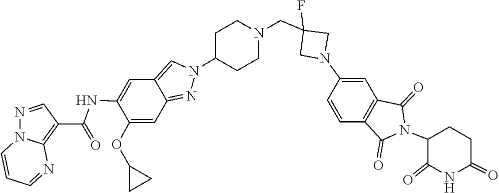 [SMILES: O=C1CCC(N2C(=O)c3ccc(N4CC(F)(CN5CCC(n6cc7cc(NC(=O)c8cnn9cccnc89)c(OC8CC8)cc7n6)CC5)C4)cc3C2=O)C(=O)N1] | 761.3 | DMSO-d₆ δ = 11.08 (s, 1 H), 10.42 (s, 1 H), 9.38 (dd, J = 6.8, 1.6 Hz, 1 H), 8.90 (dd, J = 4.4, 1.6 Hz, 1 H), 8.70 (s, 2 H), 8.33 (s, 1 H), 7.69 (d, J = 8.0 Hz, 1 H), 7.38 (s, 1 H), 7.34 (dd, J = 6.8, 4.0 Hz, 1H), 6.92 (d, J = 2.0 Hz, 1 H), 6.78 (dd, J = 8.4, 2.0 Hz, 1 H), 5.07 (dd, J = 12.8,5.2 Hz, 1 H), 4.34-4.48 (m, 1 H), 4.08-4.30 (m, 5 H), 3.07 (d, J = 11.6 Hz, 2 H), 2.91-3.00 (m, 2 H), 2.82-2.90 (m, 1 H), 2.54-2.62 (m, 2 H), 2.38-2.45 (m,2 H), 1.96-2.20 (m, 5 H), 0.91-0.99 (m, 2 H), 0.87 (s, 2 H). |
| Example 200 | | |
| 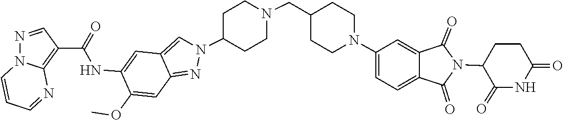 [SMILES: COc1cc2nn(C3CCN(CC4CCN(c5ccc6c(c5)C(=O)N(C5CCC(=O)NC5=O)C6=O)CC4)CC3)cc2cc1NC(=O)c1cnn2cccnc12] | 745.3 | DMSO-d₆ δ = 11.08 (s, 1 H) 10.53 (s, 1 H) 9.36 (dd, J = 7.0, 1.4 Hz, 1 H) 8.94 (dd, J = 4.2, 1.4 Hz, 1 H) 8.69 (d, J = 3.8Hz, 2H) 8.30 (s, 1H) 7.63 (d, J = 8.6 Hz, 1 H) 7.30-7.35 (m, 1 H) 7.29 (s, 1 H) 7.20 (dd, J = 8.8, 1.75 Hz, 1 H) 7.10 (s, 1 H) 5.07 (dd, J = 12.8, 5.4 Hz, 1H)4.35(br s, 1 H) 4.03 (s, 5 H) 2.86-3.01 (m, 5 H) 2.52-2.63 |
| Example 201 | | (m, 2 H) 2.18 (br d, J = 6.0 Hz, 2 H) |
| | | 2.01-2.13 (m, 7 H) 1.80(br d, J = 10.6 |
| | | Hz, 3 H) 1.15 (q, J = 11.13 Hz, 2H) |
| 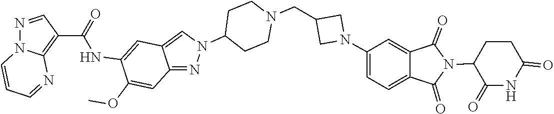 [SMILES: COc1cc2nn(C3CCN(CC4CN(c5ccc6c(c5)C(=O)N(C5CCC(=O)NC5=O)C6=O)C4)CC3)cc2cc1NC(=O)c1cnn2cccnc12] | 717.3 | DMSO-d₆ δ = 11.08 (s, 1 H), 10.55 (s, 1 H), 9.38 (dd, J = 7.2, 1.6 Hz, 1 H), 8.96 (dd, J = 4.0, 1.6 Hz, 1 H), 8.66-8.74 (m, 2 H), 8.33 (s, 1 H), 7.64 (d, J = 8.4 Hz, 1 H), 7.32-7.39 (m, 1 H), 7.12 (s, 1 H), 6.78 (d, J = 1.6 Hz, 1H), 6.65 (dd, J = 8.4, 1.6 Hz, 1 H), 5.07 |
| Example 202 | | (dd, J = 12.8, 5.2 Hz, 1H), 4.38 (s, 1 |
| | | H), 4.15 (t, J = 7.6 Hz, 2 H), 4.04 (s, 3 |
| | | H), 3.72 (s, 2 H), 2.82-3.10 (m, 4 H), |
| | | 2.64-2.73 (m, 2 H), 2.54-2.63 (m, 2H), |
| | | 2.19 (s, 2H), 1.97-2.14 (m, 5 H). |
| 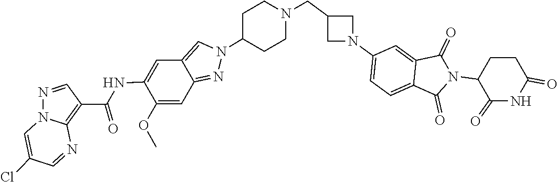 [SMILES: COc1cc2nn(C3CCN(CC4CN(c5ccc6c(c5)C(=O)N(C5CCC(=O)NC5=O)C6=O)C4)CC3)cc2cc1NC(=O)c1cnn2cc(Cl)cnc12] | 751.2 | DMSO-d₆ δ = 11.09 (s, 1 H), 10.45 (s, 1 H), 9.85 (d, J = 2.4 Hz, 1H), 9.53-9.79 (m, 1 H), 9.08 (d, J = 2.4 Hz, 1 H), 8.73 (s, 1 H), 8.70-8.72 (m, 1 H), 8.35 (s, 1 H), 7.64-7.73 (m, 1 H), 7.06-7.14 (m, 1H), 6.84 (d, J = 2.0 Hz, 1 H), 6.70 (dd, J = 8.4, 2.0 Hz, 1 H), 5.07 (dd, J = 12.8, 5.4 Hz, 1 H), 4.68-4.87 (m, 1 H), 4.27 (t, J = 8.4 Hz, 2 H), 4.05 (s, 3 H), 3.81-3.92 (m, 2 H), 3.66 (d, J = 10.8 Hz, 2 H), 3.55 (s, 2 H), 3.30-3.36 (m, 2 H), 3.23 (s, 2 H), 2.83-2.95 (m, 1 H), 2.53-2.68 (m, 3 H), 2.27-2.43 (m, 4 H), 1.96-2.08 (m, 1 H). |
| Example 203 | | |
| 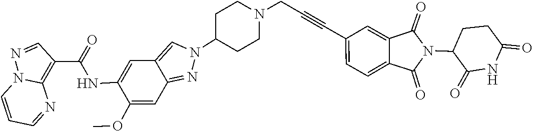 [SMILES: COc1cc2nn(C3CCN(CC#Cc4ccc5c(c4)C(=O)N(C4CCC(=O)NC4=O)C5=O)CC3)cc2cc1NC(=O)c1cnn2cccnc12] | 686.2 | DMSO-d₆ δ = 11.14 (s, 1 H), 10.54 (s, 1 H), 9.38 (dd, J = 1.6, 6.8 Hz, 1 H), 8.95 (dd, J = 1.6, 4.0 Hz, 1 H), 8.70 (d, J = 7.2 Hz, 2 H), 8.32 (s, 1 H), 8.01-7.88 (m, 3 H), 7.39-7.31 (m, 1 H), 7.13 (s, 1 H), 5.17 (dd, J = 5.2, 12.8 Hz, 1H), 4.44 (m, 1H), 4.03 (s, 3H), 3.70 (s, 2H), 3.03 (m, 2H), 2.96-2.83 (m, 1 H), 2.65-2.51 (m, 4 H), 2.21-2.04 (m, 5 H) |
| Example 204 | | |
| 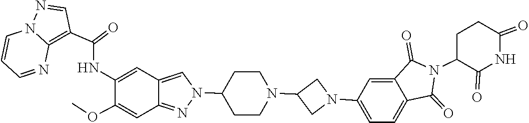 [SMILES: COc1cc2nn(C3CCN(C4CN(c5ccc6c(c5)C(=O)N(C5CCC(=O)NC5=O)C6=O)C4)CC3)cc2cc1NC(=O)c1cnn2cccnc12] | 703.2 | CDCl3 δ = 10.46 (s, 1H), 8.85 (s, 1H), 8.83 (dd, J = 1.6, 6.8 Hz, 1H), 8.79 (s, 1H), 8.75 (dd, J = 2.0, 4.4 Hz, 1H), 8.00 (s, 1H), 7.88 (s, 1H), 7.67 (d, J = 8.0 Hz, 1H), 7.07-7.03 (m, 2H), 6.83 (d, J = 2.0 Hz, 1H), 6.56 (dd, J = 2.4, 8.4 Hz, 1H), 4.98-4.90 (m, 1H), 4.48-4.37 (m, 1H), 4.16(1, J = 7.2 Hz, 2H), 4.06 (s, 3H), 3.97-3.89 (m, 2H), 3.52- |
| Example 205 | | 3.44 (m, 1H), 3.11-3.02 (m, 2H), 2.95- |
| | | 2.69 (m, 3H), 2.37-2.12 (m, 7H) |
| 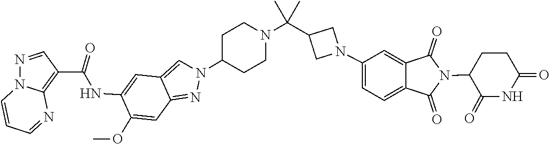 [SMILES: COc1cc2nn(C3CCN(C(C)(C)C4CN(c5ccc6c(c5)C(=O)N(C5CCC(=O)NC5=O)C6=O)C4)CC3)cc2cc1NC(=O)c1cnn2cccnc12] | 745.2 | DMSO-d₆ δ = 11.07 (br s, 1H), 10.54 (s, 1H), 9.38 (dd, J = 1.6, 7.2 Hz, 1H), 8.95 (dd, J = 1.2, 4.0 Hz, 1H), 8.69 (d, J = 7.2 Hz, 2H), 8.32-8.29 (m, 1H), 8.29-8.27 (m, 1H), 7.63 (d, J = 8.4 Hz, 1H), 7.35 (dd, J = 4.4, 7.2 Hz, 1H), 7.10 (s, 1H), 6.79 (d, J = 1.6 Hz, 1H), 6.66 (dd, J = 2.0, 8.4 Hz, 1H), 5.05 (dd, J = 4.8, 12.8 Hz, 1H), 4.38-4.27 |
| Example 206 | | (m, 1H), 4.10-4.01 (m, 5H), 3.86 (br t, |
| | | J = 7.2 Hz, 2H), 3.17-3.05 (m, 4H), |
| | | 2.93-2.83 (m, 1H), 2.63-2.55 (m, 2H), |
| | | 2.26 (br t J = 11.6 Hz, 2H),2.14- |
| | | 2.07(m, 2H), 2.05-1.96 (m, 3H), 1.06 |
| | | (s, 6H) |
| 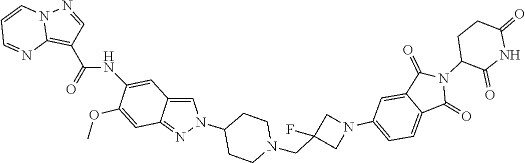 [SMILES: COc1cc2nn(C3CCN(CC4(F)CN(c5ccc6c(c5)C(=O)N(C5CCC(=O)NC5=O)C6=O)C4)CC3)cc2cc1NC(=O)c1cnn2cccnc12] | 735.2 | CDCl3 δ = 10.47 (s, 1H), 8.87-8.79 (m, 3H), 8.76 (dd, J = 1.6, 4.0 Hz, 1H), 8.16 (br s, 1H), 7.87 (s, 1H), 7.71 (d, J = 8.8 Hz, 1H), 7.09-7.03 (m, 2H), 6.87 (d, J = 2.0 Hz, 1H), 6.62 (dd, J = 2.4, 8.8 Hz, 1H), 4.95 (dd, J = 5.4, 12.2 Hz, 1H), 4.79 (br s, 1H), 4.45-4.28 (m, 1H), 4.24-4.09 (m, 4H), 4.06 (s, 3H), 3.17-3.11 (m, 1H), 3.14 (br d, J = 12.0 Hz, 2H), 2.98 (s, 1H), 2.94-2.68 (m, 4H), 2.54-2.44 (m, 2H), 2.24 (br d, J = 2.8 Hz, 4H), 2.15 (br d, J = |
| Example 207 | | 12.0 Hz, 1H) |
| 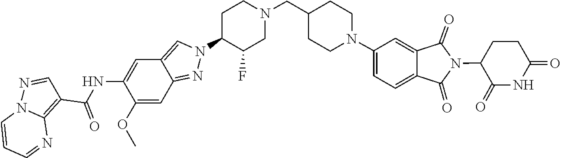 [SMILES: COc1cc2nn([C@H]3CCN(CC4CCN(c5ccc6c(c5)C(=O)N(C5CCC(=O)NC5=O)C6=O)CC4)C[C@@H]3F)cc2cc1NC(=O)c1cnn2cccnc12] | 763.1 | DMSO-d₆ δ = 11.09 (s, 1H), 10.58 (s, 1H), 9.39 (dd, J = 1.6, 7.2 Hz, 1H), 8.97 (dd, J = 1.6, 4.4 Hz, 1H), 8.76-8.68 (m, 2H), 8.40 (s, 1H), 7.69 (d, J = 8.8 Hz, 1H), 7.37 (br d, J = 4.0 Hz, 2H), 7.35 (s, 1H), 7.29 (br d, J = 8.8 Hz, 1H), 7.12 (s, 1H), 5.08 (dd, J = 5.4, 12.8 Hz, 1H), 4.99 (s, 1H), 4.12 (br d, J = 13.6 Hz, 2H), 4.06 (s, 3H), 3.17 (br s, 3H), 3.25-3.11 (m, 1H), 3.09-2.83 (m, 4H), 2.70-2.65 (m, 1H), 2.37-2.30 |
| Example 208 | | (m, 1H), 2.06-1.82 (m, 4H), 1.65-1.19 (m, |
| | | 6H) |
| 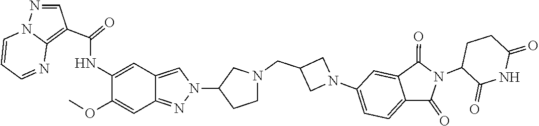 [SMILES: COc1cc2nn(C3CCN(CC4CN(c5ccc6c(c5)C(=O)N(C5CCC(=O)NC5=O)C6=O)C4)C3)cc2cc1NC(=O)c1cnn2cccnc12] | 703.1 | DMSO-d₆ δ = 11.01-11.13 (m, 1 H) 10.49-10.58 (m, 1 H) 9.32-9.43 (m, 1 H) 8.91-9.00 (m, 1 H) 8.67-8.73 (m, 2 H) 8.30-8.40 (m, 1 H) 8.10-8.15 (m, 1 H) 7.59-7.68 (m, 1H)7.29-7.41 (m, 1 H) 7.04-7.16 (m, 1 H) 6.75-6.83 (m, 1 H) 6.58-6.71 (m, 1H) 5.15-5.34 (m, 1 H) 5.01-5.09 (m, 1 H) 4.11-4.24 (m, 2 |
| Example 209 | | H) 3.97-4.07 (m,3H)3.72-3.85 (m, 2 |
| | | H) 2.97-3.25 (m, 5 H) 2.88 (ddd, |
| | | J = 17.32, 14.13, 5.44 Hz, 2 H) 2.51- |
| | | 2.62 (m, 4H) 2.23-2.33 (m, 1 H) 1.93- |
| | | 2.06 (m, 1 H) |
| 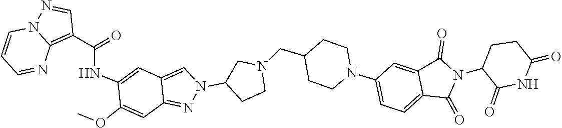 [SMILES: COc1cc2nn(C3CCN(CC4CCN(c5ccc6c(c5)C(=O)N(C5CCC(=O)NC5=O)C6=O)CC4)C3)cc2cc1NC(=O)c1cnn2cccnc12] | 731.2 | DMSO-d₆ δ = 11.03-11.11 (m, 1 H) 10.49-10.58 (m, 1 H) 9.35-9.42 (m, 1 H) 8.93-8.99 (m, 1 H) 8.64-8.74 (m, 2H) 8.28-8.34 (m, 1 H) 8.14-8.25 (m, 1 H) 7.59-7.68 (m, 1 H) 7.29-7.37 (m, 2 H) 7.20-7.27 (m, 1 H) 7.09-7.13 (m, 1 H) 4.99-5.18 (m, 2 H) 3.98-4.12 (m, 5 H) 2.92-3.03 (m, 3 H) 2.80-2.92 (m, |
| Example 210 | | 3 H) 2.53-2.64 (m, 3 H)2.29-2.41 (m, |
| | | 3H) 2.15-2.27 (m, 1 H) 1.94-2.08 (m, |
| | | 1 H) 1.74-1.91 (m, 3H) 1.09-1.28 (m, |
| | | 2 H) |
| 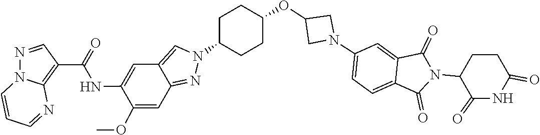 [SMILES: COc1cc2nn([C@H]3CC[C@@H](OC4CN(c5ccc6c(c5)C(=O)N(C5CCC(=O)NC5=O)C6=O)C4)CC3)cc2cc1NC(=O)c1cnn2cccnc12] | 718.2 | DMSO-d₆ δ = 11.03-11.12 (m, 1 H) 10.52 (s, 1 H) 9.38 (d, J = 6.8 Hz, l H) 8.95 (m, 1 H) 8.70 (d, J = 10.0 Hz, 2 H) 8.30 (s, 1 H) 7.67 (d, J = 8.0 Hz, l H) 7.30-7.39 (m, 1 H) 7.12 (s, 1 H) 6.84 (s, 1 H) 6.71 (d, J = 9.2 Hz, l H) 5.02-5.10 (m, 1 H) 4.58-4.67 (m, 1H) 4.40-4.51 (m, 1 H) 4.30-4.38 (m, 2 H) 4.01 (s, 3 H) 3.88-3.97 (m, 2 H) 3.70- |
| Example 211 | | 3.77 (m, 1 H) 2.16-2.29 (m, 3 H) 1.87- |
| | | 2.06 (m, 6H) 1.62-1.75 (m, 2 H) 1.22- |
| | | 1.25 (m, 1 H) |
| 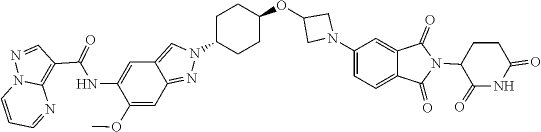 [SMILES: COc1cc2nn([C@H]3CC[C@H](OC4CN(c5ccc6c(c5)C(=O)N(C5CCC(=O)NC5=O)C6=O)C4)CC3)cc2cc1NC(=O)c1cnn2cccnc12] | 718.2 | DMSO-d₆ δ = 10.54 (s, 1 H), 9.38 (d, J = 6.8 Hz, 1 H), 8.96 (d, J = 2.4 Hz, 1 H), 8.69 (d, J = 9.6 Hz, 2 H), 8.43 (s, 1 H), 8.28 (s, 1 H), 7.66 (d, J = 8.4 Hz, 1 H), 7.40-7.29 (m, 1 H), 7.10 (s, 1H), 6.82 (s, 1H), 6.72-6.64 (m, 1 H), 5.05 (s, 1 H), 4.66 (s, 1 H), 4.51-4.27 (m, 3 H), 4.11 (m, 2 H), 4.02 (s, 3 H), 3.86- |
| Example 212 | | 3.85 (m, 2 H), 3.17-3.16 (m, 2 H), |
| | | 2.13-2.12 (m, 2 H), 1.98-1.97 (m, 3 |
| | | H), 1.49-1.48 (m, 2 H), 1.23-1.22 (m, |
| | | 2 H) |
| 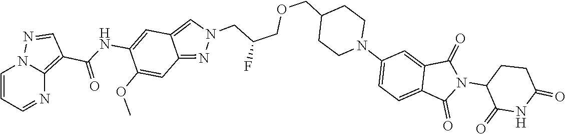 [SMILES: COc1cc2nn(C[C@@H](F)COCC3CCN(c4ccc5c(c4)C(=O)N(C4CCC(=O)NC4=O)C5=O)CC3)cc2cc1NC(=O)c1cnn2cccnc12] | 738.3 | CDCl₃ δ: 10.47 (s, 1 H) 8.81-8.87 (m, 2 H) 8.78-8.81 (m, 1 H) 8.78-8.81 (m, 1 H) 8.72-8.77 (m, 1 H) 8.08-8.17 (m, 1H) 7.87-7.95 (m, 1 H) 7.64-7.73 (m, 1 H) 7.01-7.12 (m, 3 H) 5.02-5.27 (m, 1H) 4.92-4.99 (m, 1 H) 4.61-4.76 (m, 2 H) 4.03-4.10 (m, 3 H) 3.91-4.01 (m, 2H) 3.66-3.78 (m, 1 H) 3.53-3.65 |
| Example 213 | | (m, 1 H) 3.35 (s, 2 H) 2.96-3.04 (m, 2 |
| | | H) 2.64-2.91 (m, 3 H) 2.08-2.17 (m, 1 |
| | | H) 1.86-1.95 (m, 3H)1.36-1.45 (m, |
| | | 2H) |
| 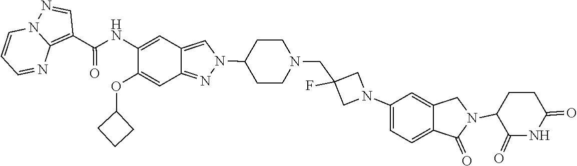 [SMILES: O=C1CCC(N2Cc3cc(N4CC(F)(CN5CCC(n6cc7cc(NC(=O)c8cnn9cccnc89)c(OC8CCC8)cc7n6)CC5)C4)ccc3C2=O)C(=O)N1] | 761.1 | DMSO-d6 δ = 10.93 (s, 1H), 10.60 (s, 1H), 9.39 (dd, J = 1.6, 7.2 Hz, 1H), 8.88 (dd, J = 1.6, 4.0 Hz, 1H), 8.72 (d, J = 6.4 Hz, 2H), 8.30 (s, 1H), 7.53 (d, J = 8.4 Hz, 1H), 7.34 (dd, J = 4.4, 7.2 Hz, 1H), 6.91 (s, 1H), 6.64 (s, 1H), 6.59 (dd, J = 1.6, 8.4 Hz, 1H), 5.05 (dd, J = 5.2, 13.2 Hz, 1H), 4.92 (t, J = 7.2 Hz, 1H), 4.39-4.29 (m, 2H), 4.24-4.18 (m, 1H), 4.13 (br dd, J = 9.6, |
| Example 214 | | 17.4 Hz, 2H), 4.06-3.97 (m, 2H), 3.06 |
| | | (br d, J = 11.6 Hz, 2H), 2.98 (s, 1H), |
| | | 2.94-2.86 (m, 2H), 2.63 (ddd, J = |
| | | 3.2, 5.6,8.4 Hz, 3H),2.45-2.36 (m, |
| | | 3H), 2.29 (ddd, J = 2.4, 7.2, 9.6 Hz, |
| | | 2H), 2.14-2.03 (m, 4H), 2.01-1.90 |
| | | (m, 2H), 1.83-1.72 (m, 1H) |
| 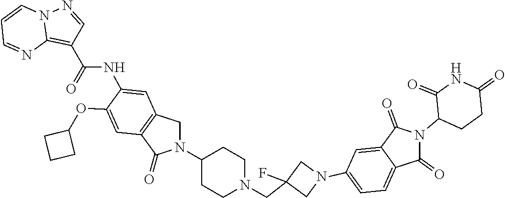 [SMILES: O=C1CCC(N2C(=O)c3ccc(N4CC(F)(CN5CCC(N6Cc7cc(NC(=O)c8cnn9cccnc89)c(OC8CCC8)cc7C6=O)CC5)C4)cc3C2=O)C(=O)N1] | 790.2 | DMSO-d₆ δ = 11.08 (s, 1 H),10.76 (s, 1 H),9.41 (d, J = 6.8Hz, 1 H),8.89 (d, J = 4.0 Hz, 1 H), 8.73 (d, J = 8.8 Hz, 2 H), 7.68 (d, J = 8.4 Hz, 1 H), 7.36 (dd, J = 6.8, 4.4 Hz, 1H), 7.10 (s, 1 H), 6.91 (s, 1 H), 6.73-6.81 (m, 1 H), 5.08 (dd, J = 12.8, 5.2 Hz, 1 H), 4.98 (q, J = 6.8 Hz, 1 H), 4.40 (s, 2 H), 4.10-4.28 (m, 4 H), 3.98 (t, J = 11.8 Hz, 1H), 3.03 (d, J = 10.4 Hz, 2 H), 2.84-2.95 (m, 3 H), 2.55-2.61 (m, 4 H), 2.21-2.37 (m, 4 H), 1.98-2.06 (m, 1 H), |
| Example 215 | | 1.87-1.95 (m, 1 H), 1.72-1.83 (m, 3 |
| | | H), 1.67 (d, J = 9.2 Hz, 2 H) |
| 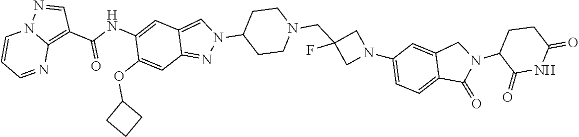 [SMILES: O=C1CCC(N2Cc3cc(N4CC(F)(CN5CCC(n6cc7cc(NC(=O)c8cnn9cccnc89)c(OC8CCC8)cc7n6)CC5)C4)ccc3C2=O)C(=O)N1] | 772.1 | DMSO-d₆ δ = 11.08 (s, 1 H),10.76 (s, 1 H),9.41 (d, J = 6.8Hz, 1 H),8.89 (d, J = 4.0 Hz, 1 H), 8.73 (d, J = 8.8 Hz, 2 H), 7.68 (d, J = 8.4 Hz, 1 H), 7.36 (dd, J = 6.8, 4.4 Hz, 1H), 7.10 (s, 1H), 6.91 (s, 1 H), 6.73-6.81 (m, 1 H), 5.08 (dd, J = 12.8, 5.2 Hz, 1 H), 4.98 (q, J = 6.8 Hz, 1 H), 4.40 (s, 2 H), 4.10-4.28 (m, 4 H), 3.98 (t, J = 11.8 Hz, 1H), 3.03, (d, J = 10.4 Hz, 2 H), 2.84- |
| Example 216 | | 2.95 (m, 3 H), 2.55-2.61 (m, 4 H), 2.21-2.37 |
| | | (m, 4 H), 1.98-2.06 (m, 1 H), 1.87- |
| | | 1.95 (m, 1 H), 1.72-1.83 (m, 3 H), |
| | | 1.67 (d, J = 9.2 Hz, 2 H) |
| 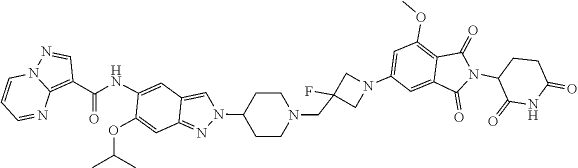 [SMILES: COc1cc(N2CC(F)(CN3CCC(n4cc5cc(NC(=O)c6cnn7cccnc67)c(OC(C)C)cc5n4)CC3)C2)cc2c1C(=O)N(C1CCC(=O)NC1=O)C2=O] | 793.5 | DMSO-d₆ δ = 11.06 (s, 1H), 10.60 (s, 1H), 9.39 (dd, J = 1.6, 7.2 Hz, 1H), 8.87 (dd, J = 1.6, 4.0 Hz, 1H), 8.75 (s, 1H), 8.71 (s, 1H), 8.31 (s, 1H), 7.34 (dd, J = 4.4, 7.2 Hz, 1H), 7.13 (s, 1H), 6.53 (s, 1H), 6.28 (d, J = 1.2 Hz, 1H), 5.00 (dd, J = 5.2, 12.8 Hz, 1H), 4.85 (td, J = 6.0, 12.4 Hz, 1H), 4.45-4.35 (m, 1H), 4.33-4.14 (m, 4H), 3.92 (s, 3H), 3.31 (s, 1H), 3.10-3.03 (m, 2H), 2.99 (s, 1H), 2.93 (s, 1H), 2.90-2.81 (m, 1H), 2.44-2.38 (m, 2H), 2.17-2.04 (m, 4H), 1.99 (s, |
| Example 217 | | 1H), 1.96 (br d, J = 4.8 Hz, 1H), 1.47 |
| | | (d, J = 6.0 Hz, 6H) |
| 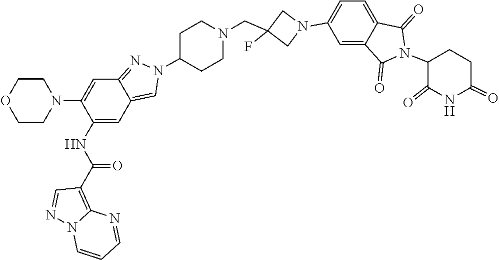 [SMILES: O=C1CCC(N2C(=O)c3ccc(N4CC(F)(CN5CCC(n6cc7cc(NC(=O)c8cnn9cccnc89)c(N8CCOCC8)cc7n6)CC5)C4)cc3C2=O)C(=O)N1] | 790.2 | DMSO-d₆ δ = 11.08 (s, 1 H), 10.67 (s, 1 H), 9.39 (dd, J = 7.2, 1.6 Hz, 1 H), 8.97 (dd, J = 4.0, 1.6 Hz, 1 H), 8.74 (d, J = 14.0 Hz, 2 H), 8.36 (s, 1 H), 7.69 (d, J = 8.4 Hz, 1 H), 7.46 (s, 1 H), 7.34-7.39 (m, 1 H), 6.92 (d, J = 2.0 Hz, 1 H), 6.78 (dd, J = 8.4, 2.0 Hz, 1 H), 5.07 (dd, J = 12.8,5.2 Hz, 1 H), 4.37-4.49 (m, 1 H), 4.13-4.30 (m, 4 H), 3.87-3.92 (m, 4 H), 3.07 (d, J = 11.2 Hz, 2 H), 2.99 (s, 1 H), 2.88-2.96 (m, 6 H), 2.55-2.63 (m, 2 H), 2.39-2.47 (m, 2 H), 2.01-2.17 (m, 5 H) |
| Example 218 | | |
| 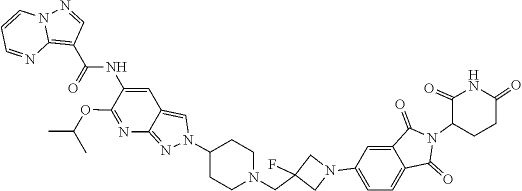 [SMILES: CC(C)Oc1nc2nn(C3CCN(CC4(F)CN(c5ccc6c(c5)C(=O)N(C5CCC(=O)NC5=O)C6=O)C4)CC3)cc2cc1NC(=O)c1cnn2cccnc12] | 764.4 | DMSO-d₆ δ = 11.08 (s, 1H), 10.61 (s, 1H), 9.40 (dd, J = 1.6, 7.2 Hz, 1H), 8.97 (s, 1H), 8.90 (dd, J = 1.6, 4.4 Hz, 1H), 8.72 (s, 1H), 8.31 (s, lH), 7.68 (d, J = 8.4 Hz, 1H), 7.42-7.30 (m, 1H), 6.92 (d, J = 1.9 Hz, 1H), 6.77 (dd, J = 2.0, 8.4 Hz, 1H), 5.51-5.36 (m, 1H), 5.07 (dd, J = 5.2, 12.8 Hz, 1H), 4.43-4.32 (m, 1H), 4.30-4.09 (m, 4H), 3.06 (d, J = 11.4 Hz, 2H), 2.99 (s, 1H), 2.95-2.80 (m, 2H), 2.59-2.53 (m, 2H), 2.45-2.37 (m, 2H), 2.18-2.00 (m, 5H), 1.51 (d, J = 6.4 Hz, 6H) |
| Example 219 | | |
| 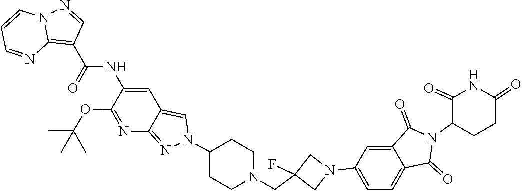 [SMILES: CC(C)(C)Oc1nc2nn(C3CCN(CC4(F)CN(c5ccc6c(c5)C(=O)N(C5CCC(=O)NC5=O)C6=O)C4)CC3)cc2cc1NC(=O)c1cnn2cccnc12] | 777.4 | DMSO-d₆ δ = 11.08 (s, 1H), 10.31 (s, 1H), 9.39 (dd, J = 1.6, 7.2 Hz, 1H), 8.85 (dd, J = 1.6, 4.4 Hz, 1H), 8.79 (s, 1H), 8.72 (s, 1H), 8.35 (s, 1H), 7.69 (d, J = 8.4 Hz, 1H), 7.37-7.32 (m, 1H), 7.28 (s, 1H), 6.92 (d, J = 2.0 Hz, 1H), 6.78 (dd, J = 2.0, 8.4 Hz, 1H), 5.07 (dd, J = 5.6, 12.8 Hz, 1H), 4.46-4.35 (m, 1H), 4.29-4.12 (m, 4H), 3.07 (br d, J = 12.0 Hz, 2H), 3.01-2.92 (m,2H), 2.91-2.80 (m, 1H), 2.60 (br d, J = 2.8 Hz, 1H), 2.57-2.55 (m, 1H), 2.45-2.39 (m, 2H), 2.18-2.00 (m, 5H), 1.53 (s, 9H) |
| Example 220 | | |
| 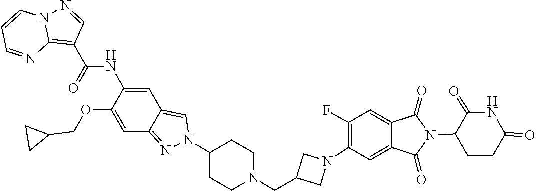 [SMILES: O=C1CCC(N2C(=O)c3cc(F)c(N4CC(CN5CCC(n6cc7cc(NC(=O)c8cnn9cccnc89)c(OCC8CC8)cc7n6)CC5)C4)cc3C2=O)C(=O)N1] | 775.4 | DMSO-d₆ δ = 11.08 (s, 1H), 10.65 (s, 1H), 9.38 (dd, J = 1.6, 7.2 Hz, 1H), 8.87 (dd, J = 1.6, 4.4 Hz, 1H), 8.74 (s, 1H), 8.71 (s, 1H), 8.31 (s, 1H),7.6O(d, J = 11.2 Hz, 1H), 7.32 (dd, J = 4.4, 7.2 Hz, 1H), 7.04 (s, 1H), 6.92 (d, J = 8.0 Hz, 1H), 5.06 (dd, J = 5.2, 12.8 Hz, 1H), 4.33-4.26 (m, 2H), 4.02 (d, J = 6.8 Hz, 2H), 3.86 (br d, J = 6.0 Hz, 2H), 3.29 (s, 4H), 3.07-2.93 (m, 3H), 2.92-2.82 (m, 1H), 2.23-2.15 (m, 2H), 2.11-2.06 (m, 3H), 2.02 (br dd, J = 3.2, 6.4 Hz, 1H), 1.53-1.46 (m, 1H), 1.29-1.19 (m, 2H), |
| Example 221 | | 0.75-0.67 (m, 2H), 0.49-0.42 (m, 2H) |
| 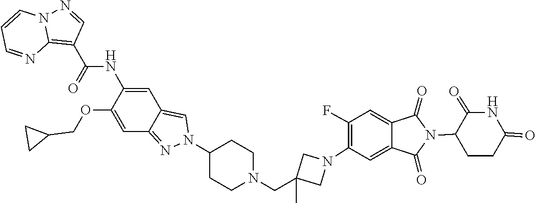 [SMILES: CC1(CN2CCC(n3cc4cc(NC(=O)c5cnn6cccnc56)c(OCC5CC5)cc4n3)CC2)CN(c2cc3c(cc2F)C(=O)N(C2CCC(=O)NC2=O)C3=O)C1] | 789.5 | DMSO-d₆ δ = 11.08 (s, 1H), 10.64 (s, 1H), 9.37 (dd, J = 1.6, 7.2 Hz, 1H), 8.86 (dd, J = 1.6, 4.4 Hz, 1H), 8.72 (d, J = 11.2 Hz, 2H), 8.29 (s, 1H), 7.59 (d, J = 11.2 Hz, 1H), 7.31 (dd, J = 4.0, 7.2 Hz, 1H), 7.03 (s, 1H), 6.91 (d, J = 7.6 Hz, 1H), 5.06 (dd, J = 5.2, 12.8 Hz, 1H), 4.36 (td, J = 5.2, 10.2 Hz, 1H), 4.02 (d, J = 7.2 Hz, 2H), 3.98-3.93 (m, 2H), 3.91-3.86 (m, 2H), 2.90-2.80 (m, 3H), 2.56 (s, 3H), 2.30-2.20 (m, 2H), 2.16-2.10 (m, 1H), 2.06 (br d, J = 3.2 Hz, 4H), 2.02-1.96 (m, 1H), 1.55-1.44 (m, 1H), 1.40 (s, 3H), 0.73-0.66 (m, 2H), 0.46 |
| Example 222 | | (q, J = 4.4 Hz, 2H) |
| 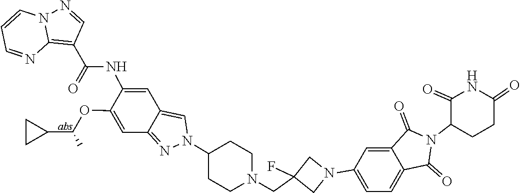 [SMILES: C[C@@H](Oc1cc2nn(C3CCN(CC4(F)CN(c5ccc6c(c5)C(=O)N(C5CCC(=O)NC5=O)C6=O)C4)CC3)cc2cc1NC(=O)c1cnn2cccnc12)C1CC1] | 789.3 | DMSO-d₆ δ = 10.08 (s, 1H), 10.63 (s, 1H), 9.40-9.38 (m, 1H), 8.89-8.87 (m, 1H), 8.76 (s, 1H), 8.72 (s, 1H), 8.31 (s, 1H), 7.70 (d, J = 8.0, 1H), 7.34-7.31 (m, 1H), 7.14 (s, 1H), 6.93 (s, 1H), 6.80-6.77 (m, 1H), 5.10-5.07 (m, 1H), 4.29-4.26 (m, 1H), 4.22-4.16 (m, 1H), 3.30 (m, 2H), 3.08-3.05 (m, 1 H), 2.99 (m, 1H), 2.93 (m, 1H), 2.57-2.55 (m, 2H), 2.45-2.42 (m, 2H), 2.11-2.06 (m, 5H), 1.46 (d, J = 6.0, 3H), 1.34-1.24 (m, 1 H), 0.57-0.55 (m, 2H), 0.44-0.39 (m, 2H) |
| Example 223 | | |
| 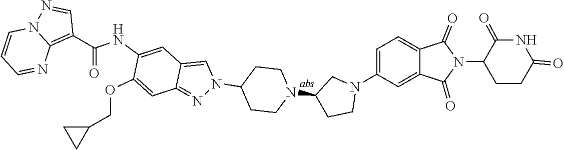 [SMILES: O=C1CCC(N2C(=O)c3ccc(N4CC[C@@H](N5CCC(n6cc7cc(NC(=O)c8cnn9cccnc89)c(OCC8CC8)cc7n6)CC5)C4)cc3C2=O)C(=O)N1] | 757.3 | DMSO-d₆ δ = 11.07 (s, 1H), 10.65 (s, 1H), 9.38 (dd, J = 1.6, 7.2 Hz, 1H), 8.87 (dd, J = 1.2, 3.6 Hz, 1H), 8.73 (d, J = 12.8 Hz, 2H), 8.32 (s, 1H), 7.65 (d, J = 8.4 Hz, 1H),7.32(dd, J = 4.4, 7.2 Hz, 1H), 7.04 (s, 1H), 6.98 (s, 1H), 6.85 (br d, J = 9.2 Hz, 1H),5.06(dd, J = 4.8, 12.4 Hz, 1H), 4.40 (br s, 1H), 4.02 (br d, J = 6.8 Hz, 2H), 3.78-3.70 (m, 1H), 3.64-3.55 (m, 1H), 3.45-3.37 |
| Example 224 | | (m, 1H), 3.25 (br t, J = 8.8 Hz, 1H), |
| | | 3.16-3.02 (m, 3H), 2.95-2.83 (m, 1H), |
| | | 2.62-2.56 (m, 2H), 2.32-2.21 (m, 3H), |
| | | 2.18-2.07 (m, 4H), 2.05-1.99 (m, 1H), |
| | | 1.93-1.87 (m, 1H), 1.56-1.42 (m, 1H), |
| | | 0.77-0.64 (m, 2H), 0.53-0.39 (m, 2H) |
| 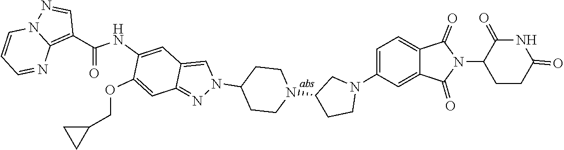 [SMILES: O=C1CCC(N2C(=O)c3ccc(N4CC[C@H](N5CCC(n6cc7cc(NC(=O)c8cnn9cccnc89)c(OCC8CC8)cc7n6)CC5)C4)cc3C2=O)C(=O)N1] | 757.3 | DMSO-d₆ δ = 11.07 (s, 1H), 10.65 (s, 1H), 9.40-9.34 (m, 1H), 8.89-8.85 (m, 1H), 8.74 (s, 1H), 8.71 (s, 1H), 8.32 (s, 1H), 7.65 (d, J = 8.8 Hz, 1H), 7.32 (dd, J = 4.4, 7.2 Hz, 1H), 7.04 (s, 1H), 6.98 (s, 1H), 6.85 (br d, J = 8.8 Hz, 1H), 5.06 (dd, J = 4.8, 12.8 Hz, 1H), 4.46-4.35 (m, 1H), 4.02 (br d, J = 6.8 Hz, 2H), 3.78-3.70 (m, 1H), 3.64- 3.55 (m, 1H), 3.45-3.38 (m, 1H), 3.28- |
| Example 225 | | 3.23 (m, 1H), 3.16-3.01 (m, 3H), |
| | | 2.94-2.82 (m, 1H), 2.62-2.55 (m, 2H), |
| | | 2.32-2.22 (m, 3H), 2.17-2.07 (m, 4H), |
| | | 2.05-1.99 (m, 1H), 1.95-1.87 (m, 1H), |
| | | 1.54-1.44 (m, 1H), 0.75-0.67 (m, 2H), |
| | | 0.50-0.42 (m, 2H) |
| 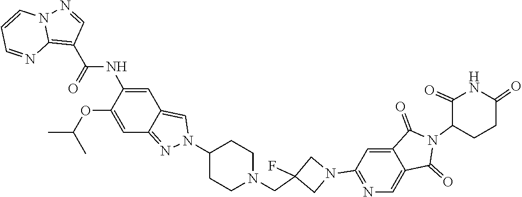 [SMILES: CC(C)Oc1cc2nn(C3CCN(CC4(F)CN(c5cc6c(cn5)C(=O)N(C5CCC(=O)NC5=O)C6=O)C4)CC3)cc2cc1NC(=O)c1cnn2cccnc12] | 762.2 | DMSO-d₆ δ = 11.08 (s, 1H), 9.79 (s, 1H), 9.38 (d, J = 7.2 Hz, 1H), 8.86 (d, J = 4.0 Hz, 1H), 8.70 (s, 1H), 8.20 (s, 1H), 8.11 (s, 1H), 7.69 (d, J = 8.4 Hz, 1H), 7.33 (dd, J = 4.4, 7.2 Hz, 1H), 6.92 (s, 1H), 6.78 (d, J = 8.4 Hz, 1H), 6.72 (s, 1H), 5.07 (dd, J = 5.2, 12.8 Hz, 1H), 4.66 (d, J = 6.4 Hz, 1H), 4.43-4.10 (m, 5H), 3.67-3.52 (m, 1H), 3.11-2.82 (m, 5H), 2.57-2.54 (m, 1H), 2.45-2.36 (m, 2H), 2.18-1.95 (m, 5H), 1.24 (d, J = 6.0 Hz, 6H), 0.90-0.80 (m, 1H) |
| Example 226 | | |
| 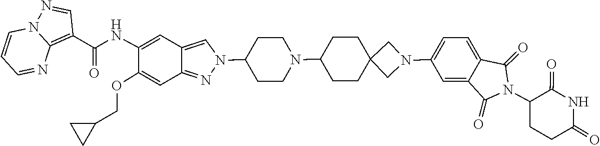 [SMILES: O=C1CCC(N2C(=O)c3ccc(N4CC5(CCC(N6CCC(n7cc8cc(NC(=O)c9cnn%10cccnc9%10)c(OCC9CC9)cc8n7)CC6)CC5)C4)cc3C2=O)C(=O)N1] | 811.2 | DMSO-d₆ δ = 11.06 (br s, 1H), 10.64 (br s, 1H), 9.37 (br d, J = 6.4 Hz, 1H), 8.86 (br s, 1H), 8.74 (s, 1H), 8.71 (s, 1H), 8.29 (s, 1H), 7.63 (br d, J = 8.0 Hz, 1H), 7.35-7.26 (m, 1H), 7.03 (br s, 1H), 6.76 (br s, 1H), 6.63 (br d, J = 8.0 Hz, 1H), 5.10-5.00 (m, 1H), 4.39-4.26 (m, 1H), 4.02 (br d, J = 6.4 Hz, 2H), 3.76 (br s, 2H), 3.69 (br s, 2H), 3.16 (br d, J = 4.0 Hz, 1H), 3.02-2.92 (m, 2H), 2.90-2.82 (m, 1H), 2.60 (br d, J = |
| Example 227 | | 1.6 Hz, 1H), 2.42-2.27 (m, 3H), 2.07 |
| | | (br s, 2H), 2.04-1.96 (m, 4H), 1.95 (br |
| | | s, 1H), 1.78-1.67 (m, 2H), 1.60-1.44 |
| | | (m, 3H), 1.42-1.26 (m, 2H), 0.71 (br d, |
| | | J = 6.8 Hz, 2H), 0.46 (br s, 2H) |
| 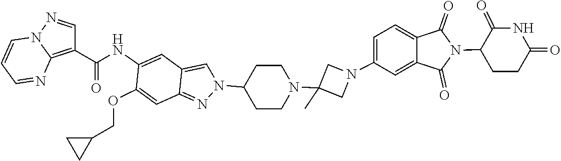 [SMILES: CC1(N2CCC(n3cc4cc(NC(=O)c5cnn6cccnc56)c(OCC5CC5)cc4n3)CC2)CN(c2ccc3c(c2)C(=O)N(C2CCC(=O)NC2=O)C3=O)C1] | 757.1 | DMSO-d₆ δ = 11.09 (s, 1 H), 10.65 (s, 1 H), 9.37 (dd, J = 6.8, 1.6 Hz, 1 H), 8.86 (dd, J = 4.0, 1.6 Hz, 1 H), 8.72 (d, J = 9.2 Hz, 2 H), 8.30 (s, 1 H), 7.66 (d, J = 8.0 Hz, 1 H), 7.31 (dd, J = 6.8, 4.4 Hz, 1 H), 7.02 (s, 1 H), 6.82 (d, J = 1.6 Hz, 1 H), 6.68 (dd, J = 8.4, 2.0 Hz, 1 H), 5.07 (dd, J = 12.8, 5.2 Hz, 1 H), 4.38 (t, J = 10.8 Hz, 1 H), 4.01 (d, J = 6.8 Hz, 2 H), 3.76-3.86 (m, 4 H), 2.79-2.93 (m, 3 H), 2.54-2.61 (m, 2 H), 2.34 (t, J = 10.8 Hz, 2 H), 2.00-2.14 (m, 5 H), 1.43-1.54 |
| Example 228 | | (m, 1 H), 1.35 (s, 3 H), 0.66-0.74 (m, 2 H), |
| | | 0.45 (q, J = 4.8 Hz, 2 H) |
| 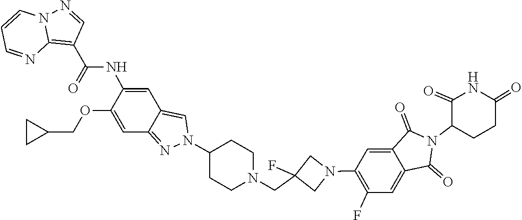 [SMILES: O=C1CCC(N2C(=O)c3cc(F)c(N4CC(F)(CN5CCC(n6cc7cc(NC(=O)c8cnn9cccnc89)c(OCC8CC8)cc7n6)CC5)C4)cc3C2=O)C(=O)N1] | 793.3 | DMSO-d₆ δ = 11.09 (s, 1 H), 10.65 (s, 1H), 9.38 (dd, J = 1.2, 7.2 Hz, 1H), 8.87 (d, J = 4.0 Hz, 1H), 8.74 (s, 1H), 8.71 (s, 1H), 8.31 (s, 1H), 7.66 (d, J = 10.8 Hz, 1H), 7.34-7.29 (m, 1H), 7.06 (d, J = 7.6 Hz, 1H), 7.04 (s, 1H), 5.11-5.04 (m, 1H), 5.14-5.02 (m, 1H), 4.44-4.22 (m, 5H), 4.02 (d, J = 6.8 Hz, 2H), 3.90 (s, 2H), 3.05 (br d, J = 12.4 Hz, 2H),2.99 (s, 1H), 2.93 (s, 1H), 2.90-2.83 (m, 1H), 2.61 (br d, J = 1.6 Hz, 1H), 2.56 (br s, 1H), 2.46-2.36 (m, 2H), 2.19-1.97 (m, 5H), 1.58-1.42 (m, 1H), 0.71 (br d, J = 7.2 Hz, 2H), 0.46 (br d, J = 5.2 Hz, 2H) |
| Example 229 | | |
| 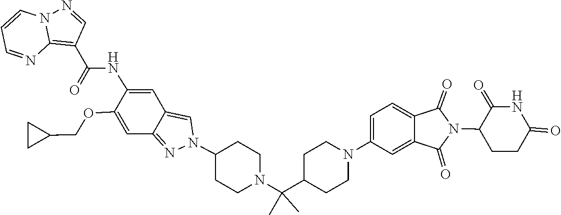 [SMILES: CC(C)(C1CCN(c2ccc3c(c2)C(=O)N(C2CCC(=O)NC2=O)C3=O)CC1)N1CCC(n2cc3cc(NC(=O)c4cnn5cccnc45)c(OCC4CC4)cc3n2)CC1] | 813.3 | DMSO-d₆ δ = 11.09 (s, 1H), 10.65 (s, 1H), 9.38 (d, J = 7.0 Hz, 1H), 8.87 (d, J = 4.2 Hz, 1H), 8.74 (s, 1H), 8.71 (s, 1H), 8.30 (s, 1H), 7.65 (d, J = 8.6 Hz, 1H), 7.34-7.29 (m, 2H), 7.23 (br d, J = 8.8 Hz, 1H), 7.03 (s, 1H), 5.07 (dd, J = 5.4, 13.0 Hz, 1H), 4.39-4.26 (m, 1H), 4.14 (br d, J = 12.0 Hz, 2H), 4.02 (d, J = 6.8 Hz, 2H), 3.11 (br d, J = 9.0 Hz, 2H), 2.96-2.82 (m, 3H), 2.62-2.55 (m, 2H), 2.24 (br t, J = 11.0 Hz, 2H), 2.11 (br d, J = 9.8 Hz, 2H), 2.00 (br dd, J = 8.0 11.8 Hz, 3H), 1.92-1.77 (m, 3H), 1.56-1.45 (m, 1H), 1.34-1.19 (m, 2H), 0.91 (s, 6H), 0.74-0.68 (m, 2H), 0.46 (q, J = 4.4 Hz, |
| Example 230 | | 2H) |
| 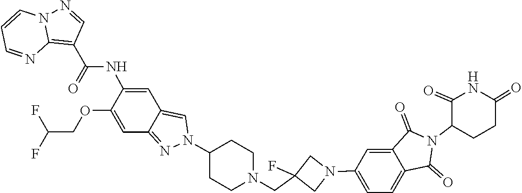 [SMILES: O=C1CCC(N2C(=O)c3ccc(N4CC(F)(CN5CCC(n6cc7cc(NC(=O)c8cnn9cccnc89)c(OCC(F)F)cc7n6)CC5)C4)cc3C2=O)C(=O)N1] | 785.2 | DMSO-d₆ δ = 10.60 (s, 1 H) 9.38 (dd, J = 7.2, 1.6 Hz, 1 H) 8.87 (dd, J = 4.0, 1.2 Hz, 1 H) 8.73 (d, J = 18.0 Hz, 3 H) 8.32 (s, 1 H) 7.34 (dd, J = 7.2, 4.4 Hz, 1 H) 7.14 (s, 1H) 6.70 (br s, 1 H) 4.86 (dt, J = 12.4, 6.4 Hz, 1 H) 4.47 (br s, 1 H) 4.14- 4.38 (m, 5 H) 2.16 (br s, 4 H) 1.48 (d, J = 6.0 Hz, 6 H) |
| Example 231 | | |
| 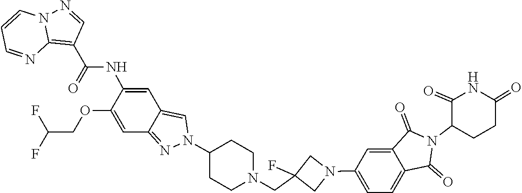 [SMILES: O=C1CCC(N2C(=O)c3ccc(N4CC(F)(CN5CCC(n6cc7cc(NC(=O)c8cnn9cccnc89)c(OCC(F)F)cc7n6)CC5)C4)cc3C2=O)C(=O)N1] | 781.2 | DMSO-d₆ δ = 11.07 (s, 1H), 10.59 (s, 1H), 9.38 (dd, J = 1.6, 7.2 Hz, 1H), 8.86 (dd, J = 1.2, 4.0 Hz, 1H), 8.77 (s, 1H), 8.71 (s, 1H), 8.35 (s, 1H), 7.64 (d, J = 8.4 Hz, 1H), 7.36 (dd, J = 4.4, 7.2 Hz, 1H), 7.22 (s, 1H), 6.79 (d, J = 1.6 Hz, 1H), 6.72-6.28 (m, 1H), 5.06 (dd, J = 5.2, 12.8 Hz, 1H), 4.60-4.47 (m, 2H), 4.45-4.34 (m, 1H), 3.85-3.73 (m, 4H), 2.95-2.81 (m, 3H), 2.65-2.53 (m, 4H), 2.28 (br t, J = 9.6 Hz, 2H), 2.13 (br s, 1H), 2.08 (br d, J = 3.6 Hz, 3H), 2.01-1.97 (m, 1H), 1.42-1.37 (m, 3H) |
| Example 232 | | |
| 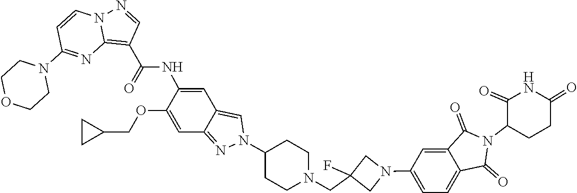 [SMILES: O=C1CCC(N2C(=O)c3ccc(N4CC(F)(CN5CCC(n6cc7cc(NC(=O)c8cnn9ccc(N%10CCOCC%10)nc89)c(OCC8CC8)cc7n6)CC5)C4)cc3C2=O)C(=O)N1] | 860.2 | DMSO-d₆ δ = 11.08 (s, 1H), 9.71 (s, 1H), 8.84 (d, J = 8.0 Hz, 1H), 8.52 (s, 1H), 8.31 (s, 1H), 8.30 (s, 1H), 7.69 (d, J = 8.4 Hz, 1H), 7.18 (s, 1H), 6.95-6.89 (m, 2H), 6.78 (dd, J = 2.0, 8.4 Hz, 1H), 5.07 (dd, J = 5.2, 12.8 Hz, 1H), 4.46-4.32 (m, 1H), 4.29-4.23 (m, 1H), 4.21 (br d, J = 4.0 Hz, 1H), 4.15 (br t, J = 10.4 Hz, 1H), 4.05 (d, J = 6.8 Hz, 2H), 3.82 (br d, J = 1.2 Hz, 4H), 3.76 (br d, J = 4.4 Hz, 4H),3.06 (br d, J = 10.4Hz, 2H), 2.99 (s, 1H), 2.92 (br s, 1H), 2.90-2.82 (m, 1H), 2.58-2.53 (m, 1H), |
| Example 233 | | 2.45 (br d, J = 5.2 Hz, 1H), 2.43-2.37 (m, |
| | | 2H), 2.16-1.96 (m, 5H), 1.28-1.14 (m, 2H), |
| | | 0.53-0.45 (m, 2H), 0.33-0.26 (m, 2H) |
| 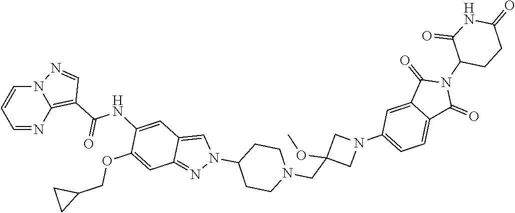 [SMILES: COC1(CN2CCC(n3cc4cc(NC(=O)c5cnn6cccnc56)c(OCC5CC5)cc4n3)CC2)CN(c2ccc3c(c2)C(=O)N(C2CCC(=O)NC2=O)C3=O)C1] | 787.2 | DMSO-d₆ δ = 11.09 (s, 1H), 10.65 (s, 1H), 9.38 (dd, J = 1.2, 6.8 Hz, 1H), 8.87 (dd, J = 1.6, 4.0 Hz, 1H), 8.74 (s, 1H), 8.71 (s, 1H), 8.30 (s, 1H), 7.66 (d, J = 8.4 Hz, 1H), 7.32 (dd, J = 4.4, 7.2 Hz, 1H), 7.03 (s, 1H), 6.85 (d, J = 2.0 Hz, 1H), 6.72 (dd, J = 2.0, 8.4 Hz, 1H), 5.07 (dd, J = 5.2, 12.8 Hz, 1H), 4.41-4.29 (m, 1H), 4.06-3.93 (m, 6H), 3.29 (s, 3H), 3.06 (br d, J = 11.2 Hz, 2H), 2.94-2.83 (m, 1H), 2.80 (s, 2H), 2.63-2.52 (m, 2H), 2.39-2.30 (m, 2H), 2.14-1.96 (m, 5H), 1.57-1.42 (m, 1H), 0.76-0.66 (m, 2H), 0.46 (q, J = 4.8 Hz, 2H) |
| Example 234 | | |
| 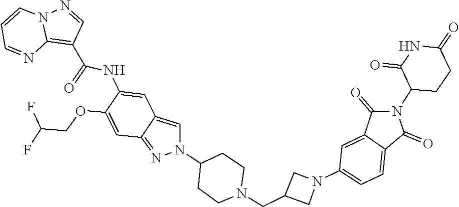 [SMILES: O=C1CCC(N2C(=O)c3ccc(N4CC(CN5CCC(n6cc7cc(NC(=O)c8cnn9cccnc89)c(OCC(F)F)cc7n6)CC5)C4)cc3C2=O)C(=O)N1] | 767.3 | DMSO-d₆ δ = 11.07 (s, 1H), 10.60 (s, 1H), 9.38 (dd, J = 1.6, 7.2 Hz, 1H), 8.86 (dd, J = 1.6, 4.0 Hz, 1H), 8.77 (s, 1H), 8.71 (s, 1H), 8.36 (s, 1H), 7.64 (d, J = 8.0 Hz, 1H), 7.36 (dd, J = 4.0, 7.2 Hz, 1H), 7.23 (s, 1H), 6.82-6.46 (m, 3H), 5.12-4.97 (m, 1H), 4.59-4.46 (m, 2H), 4.44-4.31 (m, 1H), 4.20-4.09 (m, 2H), 3.76-3.68 (m, 2H), 3.11-2.95 (m, 4H), 2.90-2.83 (m, 1H), 2.73-2.64 (m, 3H), 2.25-2.15 (m, 2H), 2.09 (br d, J = 1.2 Hz, 4H), 2.03-1.97 (m, 1H) |
| Example 235 | | |
| 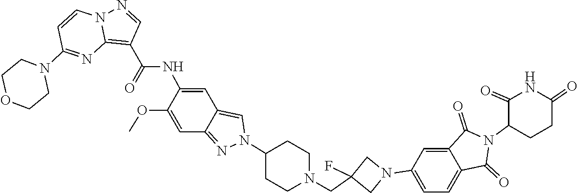 [SMILES: COc1cc2nn(C3CCN(CC4(F)CN(c5ccc6c(c5)C(=O)N(C5CCC(=O)NC5=O)C6=O)C4)CC3)cc2cc1NC(=O)c1cnn2ccc(N3CCOCC3)nc12] | 820.3 | DMSO-d₆ δ = 11.08 (br s, 1H), 9.76 (s, 1H), 8.83 (d, J = 8.0 Hz, 1H), 8.66 (s, 1H), 8.36-8.28 (m, 2H), 7.68 (d, J = 8.4 Hz, 1H), 7.10 (s, 1H), 6.91 (dd, J = 2.8, 4.8 Hz, 2H), 6.77 (dd, J = 2.0, 8.4 Hz, 1H), 5.07 (dd, J = 5.2, 12.8 Hz, 1H), 4.38 (td, J = 5.2, 10.0 Hz, 1H), 4.28-4.11 (m, 4H), 3.90 (s, 3H), 3.86 (br s, 4H), 3.80 (br d, J = 4.4 Hz, 4H), 3.05 (br d, J = 11.6 Hz, 2H), 2.98 (s, 1H), 2.92 (br s, 1H), 2.90- 2.81 (m, 1H), 2.60 (br d, J = 2.4 Hz, 1H), 2.54 (br d, J = 0.8 Hz, 1H), 2.46-2.37 |
| Example 236 | | (m, 2H), 2.14-1.98 (m, 5H) |
| 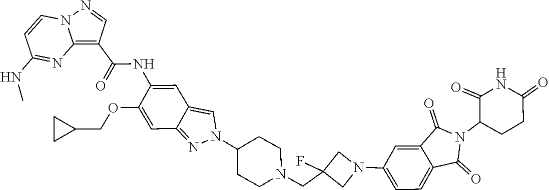 [SMILES: CNc1ccn2ncc(C(=O)Nc3cc4cn(C5CCN(CC6(F)CN(c7ccc8c(c7)C(=O)N(C7CCC(=O)NC7=O)C8=O)C6)CC5)nc4cc3OCC3CC3)c2n1] | 804.4 | DMSO-d₆ δ = 11.08 (s, 1 H), 9.93 (s, 1 H), 8.51-8.62 (m, 1 H), 8.51-8.62 (m, 2 H), 8.29 (s, 1 H), 8.21 (s, 1 H), 8.13 (s, 1 H), 7.70 (d, J = 8.4 Hz, 1 H), 7.16 (s, 1 H), 6.92 (s, 1 H), 6.78 (d, J = 8.4 Hz, 1 H), 6.40 (d, J = 7.6 Hz, 1 H), 5.07 (dd, J = 12.8, 5.2 Hz, 1 H), 4.11-4.50 (m, 5 H), 4.06 (d, J = 6.8 Hz, 2 H), 3.05 (d, J = 4.4 Hz, 6 H), 2.82-2.94 (m, 1 H), 2.52-2.63 (m, 2 H), 2.35-2.47 (m, 3 H), 1.97-2.23 (m, 5H), 1.25 (s, 1H), 0.45-0.54 (m, 2 H), 0.28-0.36 (m, 2 H) |
| Example 237 | | |
| 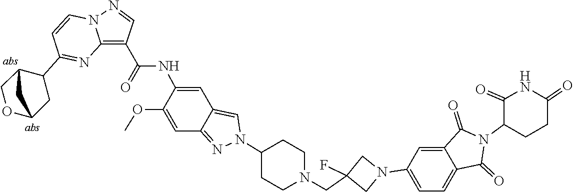 [SMILES: COc1cc2nn(C3CCN(CC4(F)CN(c5ccc6c(c5)C(=O)N(C5CCC(=O)NC5=O)C6=O)C4)CC3)cc2cc1NC(=O)c1cnn2ccc(C3C[C@H]4C[C@@H]3CO4)nc12] | 832.4 | DMSO-d₆ δ = 11.08 (s, 1H), 9.85 (br d, J = 19.2 Hz, 1H), 8.80 (d, J = 7.6 Hz, 1H), 8.69 (d, J = 1.2 Hz, 1H), 8.35-8.22 (m, 2H), 7.69 (d, J = 8.0 Hz, 1H), 7.12 (br d, J = 6.4 Hz, 1H), 6.92 (d, J = 1.6 Hz, 1H), 6.91-6.44 (m, 2H), 5.11-5.06 (m, 1H), 4.84 (br d, J = 8.8 Hz, 1H), 4.43-4.32 (m, 1H), 4.30-4.13 (m, 4H), 3.93 (s, 3H), 3.90 (s, 4H), 3.84 (br d, J = 4.0 Hz, 1H), 3.70 (br s, 1H), 3.30 (s, 1H), 3.06 (br d, J = 12.0 Hz, 2H), 3.01-2.90 (m, 2H), 2.60 (br d, J = 2.0 Hz, 1H), 2.42-2.39 (m, 1H), |
| Example 238 | | 2.15-1.99 (m, 7H) |
| 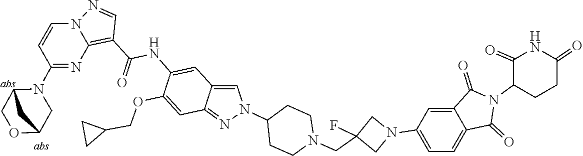 [SMILES: O=C1CCC(N2C(=O)c3ccc(N4CC(F)(CN5CCC(n6cc7cc(NC(=O)c8cnn9ccc(N%10C[C@H]%11C[C@@H]%10CO%11)nc89)c(OCC8CC8)cc7n6)CC5)C4)cc3C2=O)C(=O)N1] | 872.3 | DMSO-d6 δ = 11.08 (s, 1H), 9.88-9.72 (m, 1H), 8.79 (d, J = 7.8 Hz, 1H), 8.57-8.45 (m, 1H), 8.28 (br d, J = 8.4 Hz, 2H), 7.69 (d, J = 8.4 Hz, 1H), 7.25-7.12 (m, 1H), 6.92 (d, J = 2.0 Hz, 1H), 6.89-6.43 (m, 2H), 5.10-5.05 (m, 1H), 4.78 (br s, 1H), 4.43-4.33 (m, 1H), 4.29-4.12 (m, 4H), 4.09-4.02 (m, 2H), 3.90 (s, 4H), 3.83 (br d, J = 4.4 Hz, 1H), 3.74-3.69 (m, 1H), 3.68-3.60 (m, 1H), 3.51-3.44 (m, 1H), |
| Example 239 | | 3.06 (br d, J = 11.6 Hz, 2H),2.98 (s, 1H), |
| | | 2.95-2.82 (m, 2H), 2.54 (s, 1H), 2.46-2.37 |
| | | (m, 2H), 2.15-2.05 (m, 4H), 2.01-1.98 (m, |
| | | 2H), 1.23 (br s, 1H), 0.51 (br d, J = |
| | | 8.0 Hz, 2H), 0.30 (br d, J = 2.8 Hz, 2H) |
| 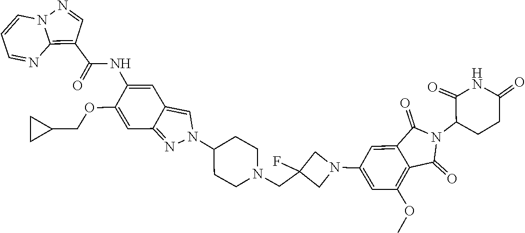 [SMILES: COc1cc(N2CC(F)(CN3CCC(n4cc5cc(NC(=O)c6cnn7cccnc67)c(OCC6CC6)cc5n4)CC3)C2)cc2c1C(=O)N(C1CCC(=O)NC1=O)C2=O] | 805.3 | DMSO-d₆ δ = 11.04 (s, 1H), 10.65 (s, 1H), 9.37 (d, J = 6.8 Hz, 1H), 8.87 (br d, J = 2.8 Hz, 1H), 8.73 (d, J = 12.4 Hz, 1H), 8.77-8.69 (m, 1H), 8.30 (s, 1H), 8.15 (s, 2H), 7.32 (dd, J = 4.0, 6.8 Hz, 1H), 7.03 (s, 1H), 6.52 (s, 1H), 6.28 (s, 1H), 5.00 (br dd, J = 5.2, 12.8 Hz, 1H), 4.45-4.33 (m, 1H), 4.32-4.15 (m, 4H), 4.02 (br d, J = 7.0 Hz, 2H), 3.92 (s, 3H), 3.06 (br d, J = 11.2 Hz, 3H), 2.99 (br s, 1H), 2.93 (br s, 1H), 2.90-2.85 (m, 1H), 2.58 (br s, 1H), 2.54 (br s, 2H), 2.42 (br s, 1H), 2.18-2.01 (m, 4H), 1.55-1.44 (m, 1H), 0.71 (br d, J = 8.0 Hz, 2H), 0.46 (br d, J = 4.4 Hz, 2H) |
| Example 240 | | |
| 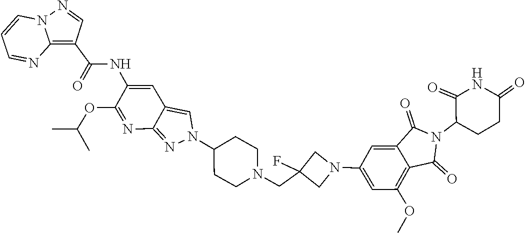 [SMILES: COc1cc(N2CC(F)(CN3CCC(n4cc5cc(NC(=O)c6cnn7cccnc67)c(OC(C)C)nc5n4)CC3)C2)cc2c1C(=O)N(C1CCC(=O)NC1=O)C2=O] | 794.3 | DMSO-d₆ δ = 11.04 (s, 1H), 10.61 (s, 1H), 9.40 (dd, J = 1.6, 7.2 Hz, 1H), 8.97 (s, 1H), 8.90 (dd, J = 1.6, 4.4 Hz, 1H), 8.73 (s, 1H), 8.31 (s, 1H), 7.40-7.32 (m, 1H), 6.52 (d, J = 1.2 Hz, 1H), 6.28 (d, J = 1.6 Hz, 1H), 5.49-5.37 (m, 1H), 5.00 (dd, J = 5.2, 12.8 Hz, 1H), 4.41-4.33 (m, 1H), 4.31-4.14 (m, 4H), 3.92 (s, 3H), 3.07 (d, J = 11.2 Hz, 2H), 3.00 (s, 1H), 2.94-2.81 (m, 2H), 2.62-2.55 (m, 1H), 2.48-2.37 (m, 3H), 2.16-2.06 (m, 4H), 2.01-1.92 (m, 1H), 1.51 (d, J = 6.0 Hz, 6H) |
| Example 241 | | |
| 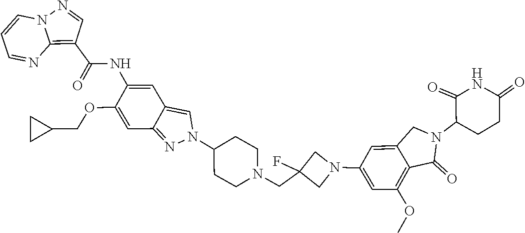 [SMILES: COc1cc(N2CC(F)(CN3CCC(n4cc5cc(NC(=O)c6cnn7cccnc67)c(OCC6CC6)cc5n4)CC3)C2)cc2c1C(=O)N(C1CCC(=O)NC1=O)C2] | 791.3 | DMSO-d₆ δ =10.88 (s, 1 H), 10.64 (s, 1 H), 9.37 (dd, J = 7.2, 1.6 Hz, 1 H), 8.87 (dd, J = 4.0, 1.6 Hz, 1 H), 8.74 (s, 1 H), 8.71 (s, 1 H), 8.30 (s, 1 H), 7.27-7.36 (m, 1H), 7.04 (s, 1H), 6.18(s, 1 H), 6.06 (s, 1 H), 4.95 (dd, J = 13.2, 5.2 Hz, 1 H), 4.33-4.44 (m, 1 H), 4.11-4.25 (m, 3 H), 3.99- 4.08 (m, 4H), 3.83 (s, 3H), 3.06 (d, J = 11.2 Hz, 2 H), 2.98 (s, 1H), 2.84-2.94 (m, 2H), 2.53-2.60 (m, 2 H), 2.38-2.46 (m, 2 H), 2.25-2.33 (m, 1 H), 2.03-2.17 (m, 4 H), 1.86-1.97 (m, 1 H), 1.50 (s, 1 H), 0.65-0.75 (m, 2 H), 0.42-0.51 (m, 2H) |
| Example 242 | | |
| 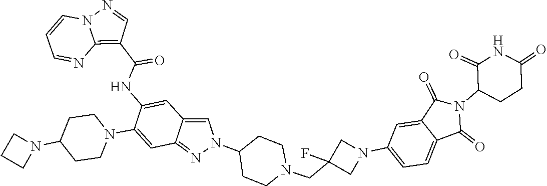 [SMILES: O=C1CCC(N2C(=O)c3ccc(N4CC(F)(CN5CCC(n6cc7cc(NC(=O)c8cnn9cccnc89)c(N8CCC(N9CCC9)CC8)cc7n6)CC5)C4)cc3C2=O)C(=O)N1] | 843.4 | DMSO-d₆ δ = 11.08 (s, 1H), 10.74 (s, 1H), 9.38 (dd, J = 1.6, 7.2 Hz, 1H), 8.93 (d, J = 2.8 Hz, 1H), 8.76 (s, 1H), 8.71 (s, 1H), 8.34 (s, 1H), 8.22 (s, 1H), 7.69 (d, J = 8.0 Hz, 1H), 7.46-7.37 (m, 2H), 6.92 (d, J = 1.6 Hz, 1H), 6.78 (dd, J = 2.0, 8.4 Hz, 1H), 5.07 (dd, J = 5.2, 12.8 Hz, 1H), 4.45-4.38 (m, 1H), 4.30-4.12 (m, 5H), 3.18 (d, J = 6.4 Hz, 4H), 3.03 (d, J = 12.4 Hz, 4H), 2.94-2.83 (m, 3H), 2.74-2.67 (m, 3H), 2.58-2.54 (m, 2H), 2.46-2.38 (m, 2H), 2.13-1.99 (m, 6H), 1.84-1.75 (m, 2H), |
| Example 243 | | 1.68-1.56 (m, 2H) |
| 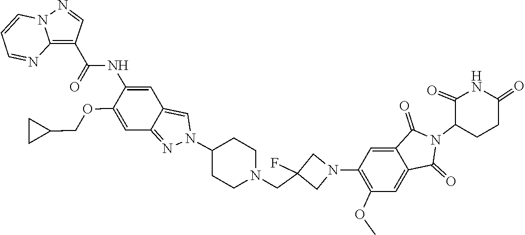 [SMILES: COc1cc2c(cc1N1CC(F)(CN3CCC(n4cc5cc(NC(=O)c6cnn7cccnc67)c(OCC6CC6)cc5n4)CC3)C1)C(=O)N(C1CCC(=O)NC1=O)C2=O] | 805.3 | DMSO-d₆ δ = 11.06 (s, 1H), 10.65 (s, 1H), 9.38 (dd, J = 1.6, 6.8 Hz, 1H), 8.87 (dd, J = 1.6, 4.4 Hz, 1H), 8.73 (d, J = 11.2 Hz, 2H), 8.31 (s, 1H), 7.34-7.28 (m, 2H), 7.04 (s, 1H), 6.83 (s, 1H), 5.05 (dd, J = 5.2, 13.2 Hz, 1H), 4.38-4.10 (m, 5H), 4.02 (d, J = 7.2 Hz, 2H), 3.91 (s, 3H), 3.08-3.01 (m, 2H),2.96 (s, 1H), 2.93-2.86 (m, 2H), 2.60 (br d, J = 2.4 Hz, 1H), 2.54 (br s, 1H), 2.45-2.35 (m, 2H), 2.15-1.99 (m, 5H), 1.55-1.41 (m, 1H), 0.78-0.61 (m, 2H), 0.50-0.43 (m, 2H) |
| Example 244 | | |
| 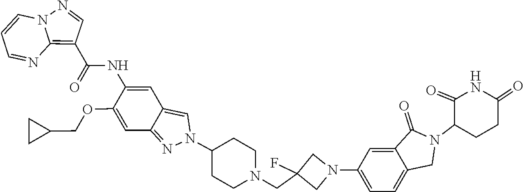 [SMILES: O=C1CCC(N2Cc3ccc(N4CC(F)(CN5CCC(n6cc7cc(NC(=O)c8cnn9cccnc89)c(OCC8CC8)cc7n6)CC5)C4)cc3C2=O)C(=O)N1] | 761.3 | DMSO-d₆ δ = 10.97 (s, 1H), 10.65 (s, 1H), 9.37 (dd, J = 1.6, 7.2 Hz, 1H), 8.87 (dd, J = 1.2, 4.0 Hz, 1H), 8.73 (d, J = 12.0 Hz, 2H), 8.30 (s, 1H), 7.45-7.38 (m, 1H), 7.32 (dd, J = 4.4, 7.2 Hz, 1H), 7.04 (s, 1H), 6.83-6.77 (m, 2H), 5.10 (dd, J = 4.8, 13.2 Hz, 1H), 4.45-4.30 (m, 2H), 4.26-4.17 (m, 1H), 4.16-3.90 (m, 7H), 3.06 (d, J = 11.2 Hz, 2H), 2.99-2.86 (m, 3H), 2.45-2.35 (m, 3H), 2.14-1.96 (m, 5H), 1.56-1.44 (m, 1H), 0.75-0.66 (m, 2H), 0.50-0.41 (m, 2H) |
| Example 245 | | |
| 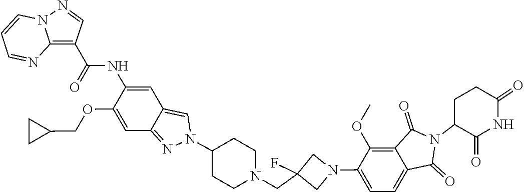 [SMILES: COc1c(N2CC(F)(CN3CCC(n4cc5cc(NC(=O)c6cnn7cccnc67)c(OCC6CC6)cc5n4)CC3)C2)ccc2c1C(=O)N(C1CCC(=O)NC1=O)C2=O] | 791.4 | DMSO-d₆ δ = 10.96 (s, 1H), 10.65 (s, 1H), 9.37 (dd, J = 1.6, 7.2 Hz, 1H), 8.87 (dd, J = 1.6, 4.0 Hz, 1H), 8.73 (d, J = 11.2 Hz, 2H), 8.31 (s, 1H), 7.35-7.27 (m, 2H), 7.04 (s, 1H), 6.63 (d, J = 8.0 Hz, 1H), 5.05 (dd, J = 5.2, 13.2 Hz, 1H), 4.50 (d, J = 16.8 Hz, 1H), 4.42-4.33 (m, 2H), 4.24-4.15 (m, 2H), 4.08 (br d, J = 9.6 Hz, 1H), 4.02 (br d, J = 6.8 Hz, 3H), 3.78 (s, 3H), 3.06 (br d, J = 11.2 Hz, 2H), 2.98 (s, 1H), 2.91 (s, 1H), 2.64-2.53 (m, 2H), 2.46-2.36 (m, 3H), 2.14-2.03 (m, 4H), 2.02-1.93 (m, 1H), |
| Example 246 | | 1.54-1.45 (m, 1H), 0.73-0.69 (m, 2H), |
| | | 0.49-0.44 (m, 2H) |
| 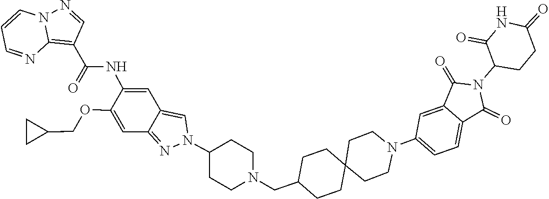 [SMILES: O=C1CCC(N2C(=O)c3ccc(N4CCC5(CCC(CN6CCC(n7cc8cc(NC(=O)c9cnn%10cccnc9%10)c(OCC9CC9)cc8n7)CC6)CC5)CC4)cc3C2=O)C(=O)N1] | 853.4 | DMSO-d₆ δ = 11.07 (s, 1 H) 10.64 (s, 1 H) 9.36 (dd, J = 7.2, 1.6 Hz, 1 H) 8.86 (dd, J = 4.4, 1.6 Hz, 1 H) 8.72 (d, J = 10.8 Hz, 2 H) 8.29 (s, 1 H) 7.64 (d, J = 8.4 Hz, 1 H) 7.25-7.34 (m, 2 H) 7.19 (br d, J = 8.8 Hz, 1 H) 7.02 (s, 1 H) 5.75 (s, 1 H) 5.06 (dd, J = 12.4, 5.2 Hz, 1 H) 4.33 (br s, 1 H) 4.01 (d, J = 6.8 Hz, 2 H) 2.93 (br s, 2 H) 2.82-2.89 (m, 1 H) 2.53-2.67 (m, 4 H) 2.17 (br d, J = 6.0 Hz, 1H) 1.95-2.12 (m, 8 H) 1.36-1.78 (m, 12 H) 1.03-1.17 (m, 4 H) 0.66-0.74 (m, 2H)0.41-0.48 (m, 2 H) |
| Example 247 | | |
| 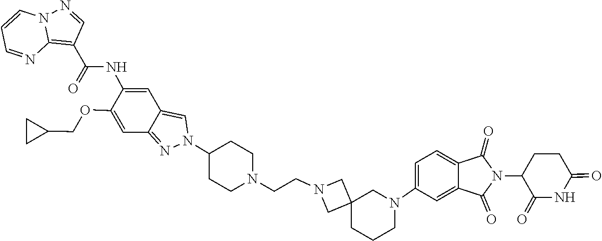 [SMILES: O=C1CCC(N2C(=O)c3ccc(N4CCCC5(CN(CCN6CCC(n7cc8cc(NC(=O)c9cnn%10cccnc9%10)c(OCC9CC9)cc8n7)CC6)C5)C4)cc3C2=O)C(=O)N1] | 840.3 | DMSO-d₆ δ = 11.08 (s, 1H), 10.65 (s, 1 H), 9.38 (dd, J = 7.00, 1.63 Hz, 1 H), 8.87 (dd, J = 4.19, 1.56 Hz, 1 H), 8.73 (d, J = 8.88 Hz, 2 H), 8.29 (s, 1 H), 8.25 (s, 1 H), 7.68 (d, J = 8.50 Hz, 1 H), 7.35-7.38 (m, 1 H), 7.31-7.35 (m, 1 H), 7.25-7.31 (m, 1 H), 7.04 (s, 1 H), 5.03-5.12(m, 1 H), 4.29-4.39 (m, 1 H), 4.00-4.05 (m, 2 H), 3.58 (s, 1 H), 3.07-3.13 (m, 3 H), 3.04 (m, 3 H), 2.95 (m, 3 H), 2.84-2.95 (m, 1 H), 2.70 (m, 3 H), 2.30-2.35 (m, 3 H), 2.00-2.17 (m, 7H), 1.72-1.80 (m, 2 H), 1.59 (br s, 2 H), 1.46-1.54 (m, 1 H), 0.68-0.75 (m, 2 H), 0.43- 0.49 (m, 2 H) |
| Example 248 | | |
| 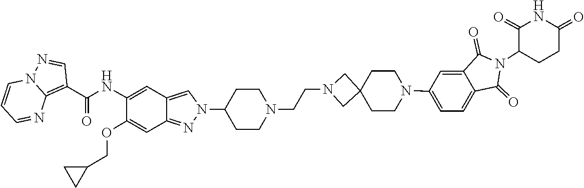 [SMILES: O=C1CCC(N2C(=O)c3ccc(N4CCC5(CC4)CN(CCN4CCC(n6cc7cc(NC(=O)c8cnn9cccnc89)c(OCC8CC8)cc7n6)CC4)C5)cc3C2=O)C(=O)N1] | 840.2 | DMSO-d₆ δ = 11.07 (s, 1H), 10.64 (s, 1H), 9.37 (dd, J = 1.6, 6.8 Hz, 1H), 8.87 (dd, J = 2.0, 4.4 Hz, 1H), 8.74 (s, 1H), 8.71 (s, 1H), 8.29 (s, 1H), 7.65 (d, J = 8.4 Hz, 1H), 7.36-7.29 (m, 2H), 7.25 (dd, J = 2.0, 8.4 Hz, 1H), 7.03 (s, 1H), 5.06 (dd, J = 5.2, 12.4 Hz, 1H), 4.40-4.30 (m, 1H), 4.02 (d, J = 7.2 Hz, 2H), 3.45 (br s, 4H), 3.19-3.05 (m, 3H), 3.03-2.97 (m, 2H), 2.93-2.83 (m, 1H), 2.66-2.53 (m, 4H), 2.41-2.34 (m, 2H), 2.19-1.97 (m, 8H), 1.76 (br s, 4H), 1.54-1.44 (m, 1H), 0.73-0.68 (m, |
| Example 249 | | 2H), 0.48-0.42 (m, 2H) |
| 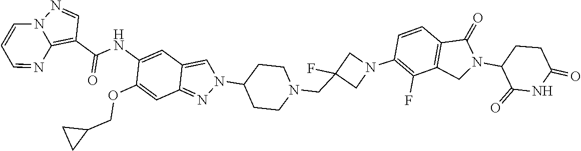 [SMILES: O=C1CCC(N2Cc3c(ccc(N4CC(F)(CN5CCC(n6cc7cc(NC(=O)c8cnn9cccnc89)c(OCC8CC8)cc7n6)CC5)C4)c3F)C2=O)C(=O)N1] | 799.2 | DMSO-d₆ δ = 10.97 (s, 1 H), 10.65 (s, 1 H), 9.37 (d, J = 7.2 Hz, 1 H), 8.86 (d, J = 3.2 Hz, 1H), 8.72 (d, J = 11.2 Hz, 2 H), 8.30 (s, 1 H), 7.42 (d, J = 8.0 Hz, 1 H), 7.31 (m, 1 H), 7.03 (s, 1 H), 6.73 (t, J = 8.0 Hz, 1 H), 5.05 (m, 1 H), 4.46 (d, J = 16.8 Hz, 1 H), 4.34-4.41 (m, 1 H), 4.21- 4.33 (m, 3 H), 4.09-4.19 (m, 2 H), 4.02 (d, J = 6.8 Hz, 2 H), 3.05 (d, J = 11.2 Hz, 2 H), 2.88-2.95 (m, 2H), 2.61 (s, 2 H), 2.41 |
| Example 250 | | (d, J = 12.0 Hz, 2 H), 1.92-2.18 (m, 6 H), |
| | | 1.50 (s, 1 H), 0.67-0.74 (m, 2 H), 0.46 |
| | | (d, J = 4.8 Hz, 2 H) |
| 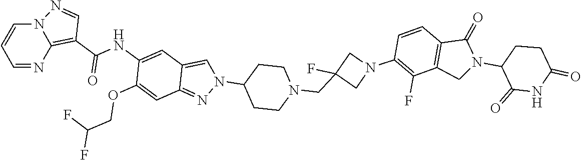 [SMILES: O=C1CCC(N2Cc3c(ccc(N4CC(F)(CN5CCC(n6cc7cc(NC(=O)c8cnn9cccnc89)c(OCC(F)F)cc7n6)CC5)C4)c3F)C2=O)C(=O)N1] | 789.2 | DMSO-d₆ δ = 10.97 (s, 1H), 10.60 (s, 1H), 9.39 (dd, J = 1.6, 7.2 Hz, 1H), 8.86 (dd, J = 1.6, 4.4 Hz, 1H), 8.77 (s, 1H), 8.72 (s, 1H), 8.36 (s, 1H), 7.42 (d, J = 8.0 Hz, 1H), 7.38-7.34 (m, 1H), 7.22 (s, 1H), 6.80-6.49 (m, 2H), 5.06 (dd, J = 4.8, 13.2 Hz, 1H), 4.57-4.44 (m, 3H), 4.43-4.36 (m, 1H), 4.33-4.21 (m, 3H), 4.19-4.10 (m, 2H), 3.06 (br d, J = 12.0 Hz, 2H), 3.00-2.93 (m, 2H), 2.92-2.85 (m, 1H), 2.58 (br dd, J = 2.8, |
| Example 251 | | 17.2 Hz, 1H), 2.44-2.38 (m, 3H), 2.16-2.05 |
| | | (m, 4H), 2.01-1.94 (m, 1H) |
| 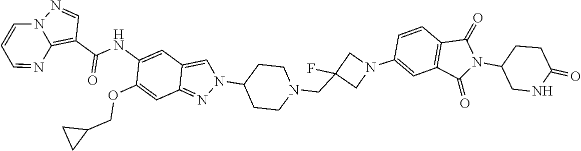 [SMILES: O=C1CCC(N2C(=O)c3ccc(N4CC(F)(CN5CCC(n6cc7cc(NC(=O)c8cnn9cccnc89)c(OCC8CC8)cc7n6)CC5)C4)cc3C2=O)CN1] | 761.3 | DMSO-d₆ δ = 10.64 (s, 1 H) 9.37 (dd, J = 7.2, 1.6 Hz, 1 H) 8.86 (dd, J = 4.4, 1.6 Hz, 1 H) 8.72 (d, J = 12.4 Hz, 2 H) 8.29 (s, 1 H) 7.60-7.67 (m, 1 H) 7.54 (d, J = 3.6 Hz, 1 H) 7.31 (dd, J = 6.8, 4.4 Hz, 1 H) 7.03 (s, 1 H) 6.84-6.90 (m, 1 H) 6.73 (dd, J = 8.4, 2.0 Hz, 1 H) 4.36 (br s, 2 H) 4.09-4.27 (m, 4 H) 4.02 (d, J = 7.2 Hz, 2 H) 3.62 (br t, J = 11.2 Hz, 1 H) 3.13-3.21 (m, 1 H) 3.05 (br d, J = 11.2 Hz, 2 H) 2.89-2.99 (m, 2H) |
| Example 252 | | 2.30-2.45 (m, 4H) 2.05-2.14 (m, 4 H) |
| | | 1.79-1.91 (m, 1 H) 1.46-1.54 (m, 1H) |
| | | 1.21-1.28 (m, 1 H) 0.68-0.74 (m, 2 H) 0.46 |
| | | (q, J = 4.8 Hz, 2H) |
| 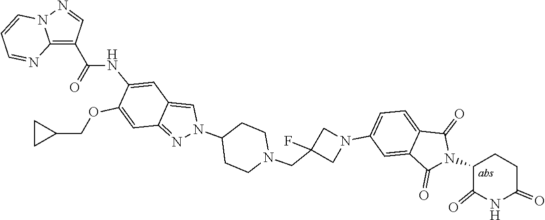 [SMILES: O=C1CC[C@@H](N2C(=O)c3ccc(N4CC(F)(CN5CCC(n6cc7cc(NC(=O)c8cnn9cccnc89)c(OCC8CC8)cc7n6)CC5)C4)cc3C2=O)C(=O)N1] | 775.2 | DMSO-d₆ δ = 11.08 (s, 1H), 10.64 (s, 1H), 9.37 (dd, J = 1.2, 6.8 Hz, 1H), 8.86 (dd, J = 1.6, 4.4 Hz, 1H), 8.74 (s, 1H), 8.71 (s, 1H), 8.30 (s, 1H), 7.68 (d, J = 8.0 Hz, 1H), 7.34-7.28 (m, 1H), 7.03 (s, 1H), 6.92 (d, J = 1.6 Hz, 1H), 6.77 (dd, J = 2.0, 8.4 Hz, 1H), 5.07 (dd, J = 5.2, 12.4 Hz, 1H), 4.42-4.32 (m, 1H), 4.30-4.11 (m, 4H), 4.05-3.99 (m, 2H), 3.06 (br d, J = 11.2 Hz, 2H), 3.00-2.91 (m, 2H), 2.90-2.82 (m, 1H), 2.63-2.53 (m, 2H), 2.45-2.37 (m, 2H), 2.17-1.99 (m, 5H), 1.55-1.45 (m, 1H), 0.75-0.67 (m, 2H), 0.46 (q, J = 4.8 Hz, 2H) |
| Example 253 | | |
| 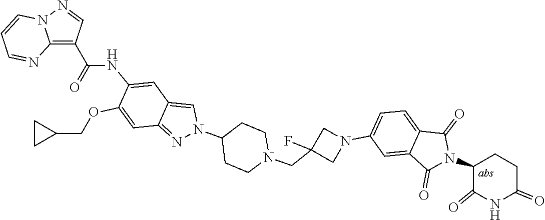 [SMILES: O=C1CC[C@H](N2C(=O)c3ccc(N4CC(F)(CN5CCC(n6cc7cc(NC(=O)c8cnn9cccnc89)c(OCC8CC8)cc7n6)CC5)C4)cc3C2=O)C(=O)N1] | 775.2 | DMSO-d₆ δ = 11.08 (s, 1H), 10.65 (s, 1H), 9.37 (dd, J = 1.6, 6.8 Hz, 1H), 8.87 (dd, J = 1.6, 4.0 Hz, 1H), 8.74 (s, 1H), 8.71 (s, 1H), 8.30 (s, 1H), 7.69 (d, J = 8.4 Hz, 1H), 7.34-7.29 (m, 1H), 7.03 (s, 1H), 6.92 (d, J = 2.0 Hz, 1H), 6.77 (dd, J = 2.0, 8.4 Hz, 1H), 5.07 (dd, J = 5.2, 12.8 Hz, 1H), 4.42-4.33 (m, 1H), 4.30-4.11 (m, 4H), 4.02 (d, J = 6.8 Hz, 2H), 3.06 (br d, J = 11.2 Hz, 2H), 3.00-2.91 (m, 2H), 2.91-2.83 (m, 1H), 2.63-2.53 (m, 2H), 2.45-2.38 (m, 2H), 2.17-1.99 (m, 5H), 1.54-1.46 (m, 1H), 0.74-0.67 (m, 2H), 0.46 (q, J = 4.8 Hz, 2H) |
| Example 254 | | |
| 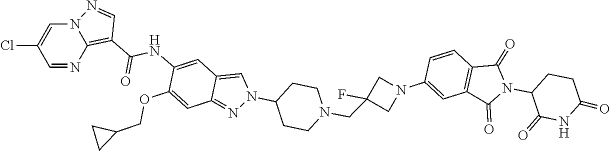 [SMILES: O=C1CCC(N2C(=O)c3ccc(N4CC(F)(CN5CCC(n6cc7cc(NC(=O)c8cnn9cc(Cl)cnc89)c(OCC8CC8)cc7n6)CC5)C4)cc3C2=O)C(=O)N1] | 809.2 | DMSO-d₆ δ = 11.04-11.11 (m, 1 H), 10.39-10.45 (m, 1 H), 9.81-9.86 (m, 1 H), 8.86-8.92 (m, 1 H), 8.70-8.76 (m, 2 H), 8.29-8.34 (m, 1 H), 7.64-7.72 (m, 1 H), 7.02-7.07 (m, 1 H), 6.89-6.94 (m, 1 H), 6.74-6.82 (m, 1 H), 5.03-5.12 (m, 1 H), 4.33-4.42 (m, 1 H), 4.12-4.29 (m, 4 H), 3.99-4.05 (m, 2 H), 3.03-3.09 (m, 2 H), 2.91-3.00 (m, 2 H), 2.89 (s, 1 H), 2.54- 2.62 (m, 1 H), 2.37-2.44 (m, 2 H), 1.99- |
| Example 255 | | 2.16 (m, 5 H), 1.46-1.56 (m, 1 H), 1.11- |
| | | 1.22 (m, 1 H), 0.66-0.73 (m, 2 H), 0.40- |
| | | 0.47 (m, 2 H) |
| 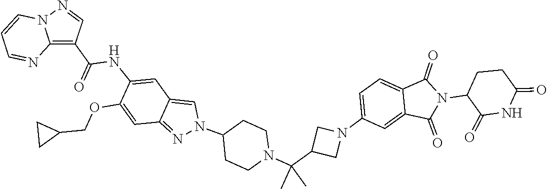 [SMILES: CC(C)(C1CN(c2ccc3c(c2)C(=O)N(C2CCC(=O)NC2=O)C3=O)C1)N1CCC(n2cc3cc(NC(=O)c4cnn5cccnc45)c(OCC4CC4)cc3n2)CC1] | 785.2 | DMSO-d₆ δ = 11.09 (s, 1H), 10.66 (s, 1H), 9.36 (dd, J = 1.6, 7.2 Hz, 1H), 8.86 (dd, J = 1.6, 4.0 Hz, 1H), 8.75 (s, 1H), 8.71 (s, 1H), 8.30 (s, 1H), 7.62 (d, J = 8.4 Hz, 1H),7.31 (dd, J = 4.4, 6.8 Hz, 1H), 7.04 (s, 1H), 6.76 (d, J = 1.6 Hz, 1H), 6.64 (dd, J = 2.0, 8.4 Hz, 1H), 5.06 (dd, J = 5.6, 12.8 Hz, 1H), 4.35-4.26 (m, 1H), 4.07-4.00 (m, 4H), 3.85 (br t, J = 7.6 Hz, 2H), 3.17-3.11 (m, 1H), 3.05 (br d, J = 10.0 Hz, 2H), 2.94-2.83 (m, 1H), 2.62-2.57 (m, 1H), 2.53 (br d, J = 2.0 Hz, 1H), 2.23 (br t, J = 10.8 |
| Example 256 | | Hz, 2H), 2.11-1.93 (m, 5H), 1.55-1.46 (m, |
| | | 1H), 1.05 (s, 6H), 0.75-0.69 (m, 2H), |
| | | 0.46 (q, J = 4.8 Hz, 2H) |
| 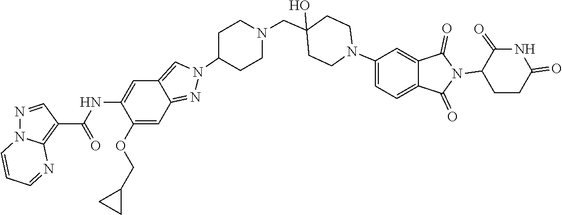 [SMILES: O=C1CCC(N2C(=O)c3ccc(N4CCC(O)(CN5CCC(n6cc7cc(NC(=O)c8cnn9cccnc89)c(OCC8CC8)cc7n6)CC5)CC4)cc3C2=O)C(=O)N1] | 801.3 | DMSO-d₆ δ = 11.16-11.02 (m, 1H), 10.71-10.58 (m, 1H), 9.46-9.29 (m, 1H), 8.91- 8.83 (m, 1H), 8.77-8.69 (m, 2H), 8.32-8.25 (m, 1H), 7.69-7.62 (m, 1H), 7.37-7.29 (m, 2H), 7.28-7.22 (m, 1H), 7.05-6.99 (m, 1H), 5.07 (dd, J = 6.0, 13.2 Hz, 1H), 4.38-4.27 (m, 1H), 4.01 (br d, J = 6.8 Hz, 2H), 3.78 (br d, J = 12.8 Hz, 2H), 3.08 (br d, J = 11.0 Hz, 2H), 2.87 (br d, J = 11.6 Hz, 1H), 2.63-2.54 (m, 3H), 2.42-2.29 (m, 5H), 2.19-1.95 (m, 6H), 1.69-1.56 (m, 4H), 1.54-1.43 (m, 1H), 0.75-0.66 (m, 2H), 0.45 (br d, J = 5.0 Hz, 2H) |
| Example 257 | | |
| 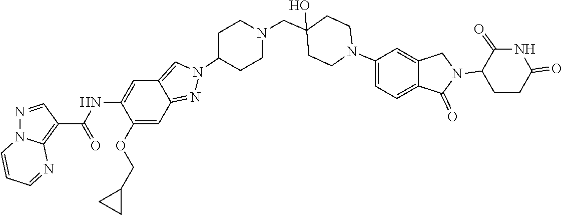 [SMILES: O=C1CCC(N2Cc3cc(N4CCC(O)(CN5CCC(n6cc7cc(NC(=O)c8cnn9cccnc89)c(OCC8CC8)cc7n6)CC5)CC4)ccc3C2=O)C(=O)N1] | 787.3 | DMSO-d₆ δ = 10.95 (s, 1H), 10.64 (s, 1H), 9.37 (dd, J = 1.6, 7.2 Hz, 1H), 8.86 (dd, J = 1.2, 4.0 Hz, 1H), 8.72 (d, J = 10.4 Hz, 2H), 8.28 (s, 1H), 7.50 (d, J = 8.8 Hz, 1H), 7.31 (dd, J = 4.4, 7.2 Hz, 1H), 7.10-7.00 (m, 3H), 5.05 (dd, J = 5.2, 13.6 Hz, 1H), 4.39-4.27 (m, 2H), 4.25-4.15 (m, 1H), 4.01 (br d, J = 7.0 Hz, 2H), 3.59 (br d, J = 12.2 Hz, 2H), 3.28-3.19 (m, 3H), 3.09 (br d, J = 11.0 Hz, 2H),2.98-2.84 (m, 1H), 2.40-2.30 (m, 5H), 2.18-1.91 (m, 6H), 1.71-1.55 (m, 4H), 1.55-1.41 (m, 1H), 0.74-0.67 (m, 2H), 0.49-0.42 (m, 2H) |
| Example 258 | | |
| 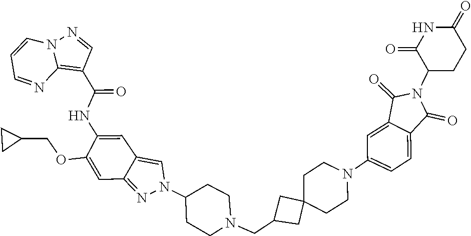 [SMILES: O=C1CCC(N2C(=O)c3ccc(N4CCC5(CC4)CC(CN4CCC(n6cc7cc(NC(=O)c8cnn9cccnc89)c(OCC8CC8)cc7n6)CC4)C5)cc3C2=O)C(=O)N1] | 825.3 | DMSO-d₆ δ = 11.07 (s, 1H), 10.64 (s, 1H), 9.37 (dd, J = 1.6, 7.2 Hz, 1H), 8.86 (dd, J = 1.6, 4.0 Hz, 1H), 8.72 (d, J = 9.6 Hz, 2H), 8.30 (s, 1H), 7.64 (d, J = 8.4 Hz, 1H), 7.36-7.29 (m, 2H), 7.27-7.19 (m, 1H), 7.03 (s, 1H), 5.06 (dd, J = 5.2, 12.8 Hz, 1H), 4.39-4.26 (m, 1H), 4.02 (d, J = 6.4 Hz, 2H), 3.50-3.44 (m, 2H), 3.41-3.37 (m, 3H), 3.00-2.82 (m, 3H), 2.62-2.52 (m, 3H), 2.20-1.91 (m, 10H), 1.70-1.61 (m, 2H), 1.58-1.42 (m, 5H), 0.75-0.65 (m, 2H), 0.50-0.40 (m, 2H) |
| Example 261 | | |
| 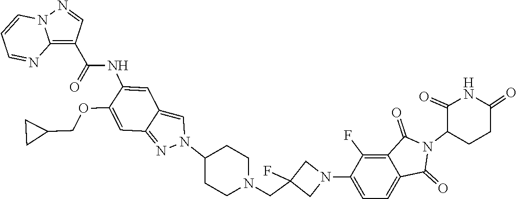 [SMILES: O=C1CCC(N2C(=O)c3ccc(N4CC(F)(CN5CCC(n6cc7cc(NC(=O)c8cnn9cccnc89)c(OCC8CC8)cc7n6)CC5)C4)c(F)c3C2=O)C(=O)N1] | 793.3 | DMSO-d₆ δ = 11.10 (s, 1 H), 10.65 (s, 1 H), 9.38 (dd, J = 6.8, 1.6 Hz, 1 H), 8.87 (dd, J = 4.4, 1.6 Hz, 1 H), 8.74 (s, 1 H), 8.71 (s, 1 H), 8.31 (s, 1 H), 7.57 (d, J = 8.0 Hz, 1H), 7.28-7.35 (m, 1 H), 7.03 (s, 1 H), 6.90 (1, J = 8.0 Hz, 1H), 5.07 (dd, J = 12.8, 5.6 Hz, 1 H), 4.23-4.40 (m, 5 H), 4.02 (d, J = 6.8 Hz, 2 H), 3.06 (d, J = 11.2 Hz, 2 H), 2.92-3.00 (m, 2 H), 2.81-2.90 (m, 1 H), 2.60 (d, J = 2.8 Hz, 2 H), 2.39 (s, 2 H), 2.02-2.15 (m, 5 H), 1.45-1.55 (m, 1 H), 0.66-0.76 (m, 2 H),0.46 (q, J = 4.8 Hz, 2 H). |
| Example 262 | | |
| 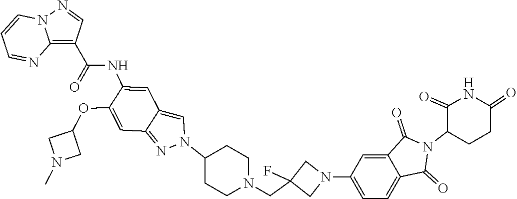 [SMILES: CN1CC(Oc2cc3nn(C4CCN(CC5(F)CN(c6ccc7c(c6)C(=O)N(C6CCC(=O)NC6=O)C7=O)C5)CC4)cc3cc2NC(=O)c2cnn3cccnc23)C1] | 790.3 | DMSO-d₆ δ = 11.08 (s, 1 H) 10.57 (s, 1 H) 9.40 (dd, J = 6.8, 1.2 Hz, 1 H) 8.90 (dd, J = 4.4, 1.6 Hz, 1 H) 8.73 (d, J = 6.8 Hz, 2H) 8.33 (s, 1 H) 8.23 (s, 1 H) 7.69 (d, J = 8.4 Hz, 1 H) 7.33-7.38 (m, 1H) 6.92 (d, J = 2.0 Hz, 1 H) 6.87 (s, 1 H) 6.78 (dd, J = 8.4, 2.0 Hz, 1 H) 5.07 (dd, J = 12.8, 5.2 Hz, 1 H) 4.98 (1, J = 5.6 Hz, 1 H) 4.37 (br d, J = 4.0 Hz, 1 H) 4.26 (br t, J = 10.0 Hz, 2 H) 4.21 (br d, J = 4.0 Hz, 1 H) 4.15 (br t, J = 11.2 Hz, 2 H) 3.94 (br t, J = 7.6 Hz, 3 H) 3.15-3.27 (m, 2 H) 3.06 (br d, J = 11.6 Hz, 2 H) 2.99 (s, 1H)2.93 (br s, 1H)2.67 |
| Example 263 | | (d, J = 2.0 |
| | | Hz, 1 H) 2.61 (br s, 1H)2.41 (br s, 1 H) |
| | | 2.37 (s, 3 H) 2.32-2.34 (m, 1 H) |
| | | 2.04-2.12 (m, 4 H) |
| 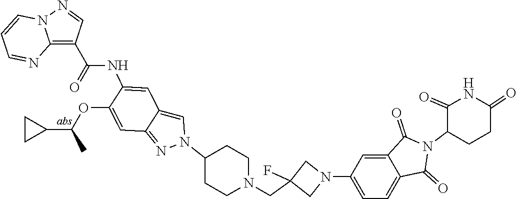 [SMILES: C[C@H](Oc1cc2nn(C3CCN(CC4(F)CN(c5ccc6c(c5)C(=O)N(C5CCC(=O)NC5=O)C6=O)C4)CC3)cc2cc1NC(=O)c1cnn2cccnc12)C1CC1] | 789.3 | DMSO-d₆ δ = 10.08 (s, 1H), 10.63 (s, 1H), 9.40-9.38 (m, 1H), 8.89-8.87 (m, 1H), 8.76 (s, 1H), 8.72 (s, 1H), 8.31 (s, 1H), 7.70 (d, J = 8.0, 1H), 7.34-7.31 (m, 1H), 7.14 (s, 1H), 6.93 (s, 1H), 6.80-6.77 (m, 1H), 5.10-5.07 (m, 1H), 4.29-4.26 (m, 1H), 4.22-4.16 (m, 1H), 3.30 (m, 2H), 3.08-3.05 (m, 1 H), 2.99 (m, 1H), 2.93 (m, 1H), 2.57-2.55 (m, 2H), 2.45-2.42 (m, 2H), 2.11-2.06 (m, 5H), 1.46 (d, J = 6.0, 3H), 1.34-1.24 (m, 1 H), 0.57-0.55 (m, 2H), 0.44-0.39 (m, 2H) |
| Example 264 | | |

### Preparation of Example 265: N-(6-(cyclopropylmethoxy)-2-(1-((1-(2-(2,6-dioxopiperidin-3-yl)-1,3-dioxoisoindolin-4-yl)-3-fluoroazetidin-3-yl)methyl)piperidin-4-yl)-2H-indazol-5-yl)pyrazolo[1,5-a]pyrimidine-3-carboxamide

### Step 1:

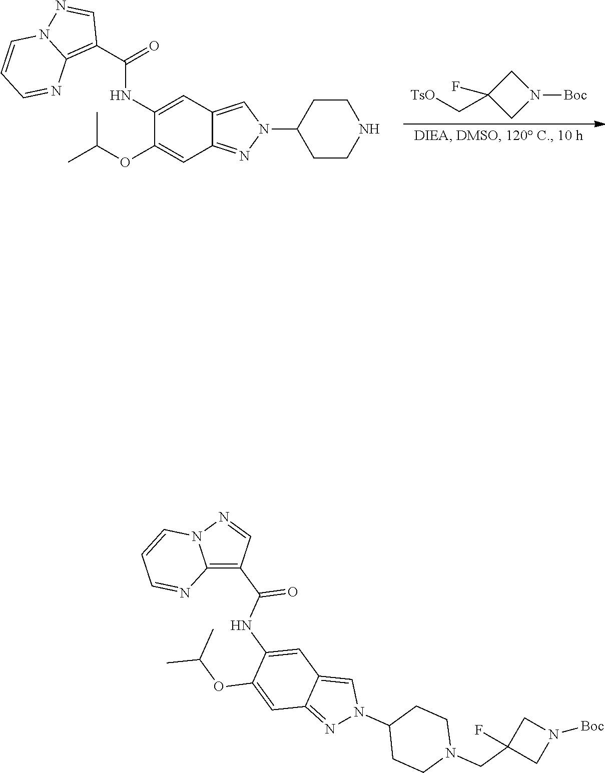

To a solution of N-(6-isopropoxy-2-(piperidin-4-yl)-2H-indazol-5-yl)pyrazolo[1,5-a]pyrimidine-3-carboxamide (200 mg, 476. 8 μmol, 1 eq) and diisopropylethylamine (282.2 mg, 2.18 mmol, 380.35 μL, 4.58 eq) in dimethyl sulfoxide (5 mL) was added tert-butyl 3-fluoro-3-((tosyloxy)methyl)azetidine-1-carboxylate (455.8 mg, 1.27 mmol, 2.66 eq) at 25° C. The mixture was stirred at 100° C. for 12 h. The reaction mixture was quenched by the addition of water (50 mL) at 0° C., and then extracted with dichloromethane (30 mL×3). The combined organic layers were washed with brine (10 mL×3), dried over anhydrous sodium sulfate, filtered, and concentrated under reduced pressure to give a residue. The residue was purified by preparative thin layer chromatography (silicon dioxide, petroleum ether/ethyl acetate=0:1) to afford tert-butyl 3-fluoro-3-[[4-[6-isopropoxy-5-(pyrazolo[1,5-a]pyrimidine-3-carbonylamino)indazol-2-yl]-1-piperidyl]methyl]azetidine-1-carboxylate (60 mg, 98.9 μmol, 20% yield) as a yellow solid. MS (ESI) m/z: 607.2 [M+H]⁺.

### Step 2:

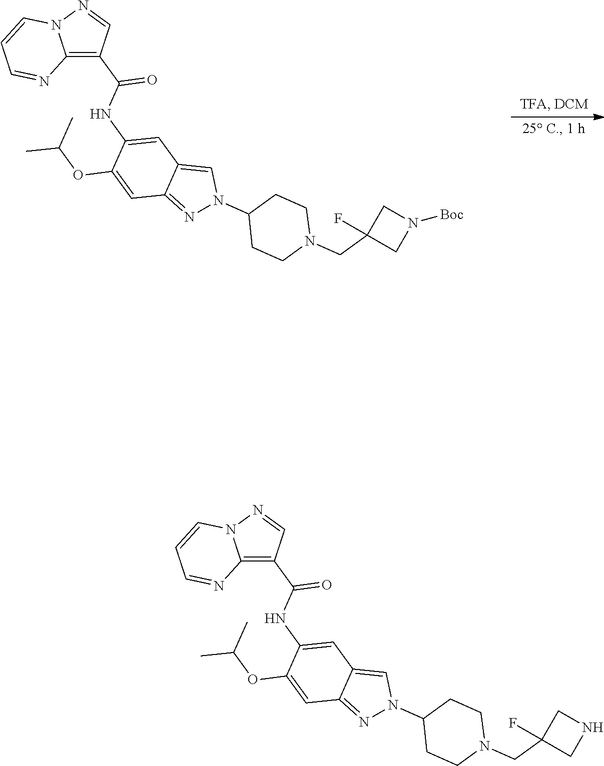

A solution of tert-butyl 3-fluoro-3-[[4-[6-isopropoxy-5-(pyrazolo[1,5-a]pyrimidine-3-carbonylamino)indazol-2-yl]-1-piperidyl]methyl]azetidine-1-carboxylate (120 mg, 197.8 μmol, 1 eq) and trifluoroacetic acid (22.55 mg, 197.8 μmol, 14.6 uL, 1 eq) in dichloromethane (10 mL) was allowed to stir at 25° C. for 1 h. The reaction mixture was concentrated in vacuo and then resuspended in a 10:1 mixture of dichloromethane:methanol (100 mL). The pH of the solution was adjusted to 8 by addition of ammonium hydroxide. The mixture was extracted and the combined organic phases were dried over anhydrous sodium sulfate, filtered, and concentrated under reduced pressure to afford N-[2-[1-[(3-fluoroazetidin-3-yl)methyl]-4-piperidyl]-6-isopropoxy-indazol-5-yl]pyrazolo[1,5-a]pyrimidine-3-carboxamide (100 mg, 197.40 μmol, 99% yield) as a yellow solid which was used in the next step without further purification. MS (ESI) m/z: 507.1 [M+H]⁺.

### Step 3:

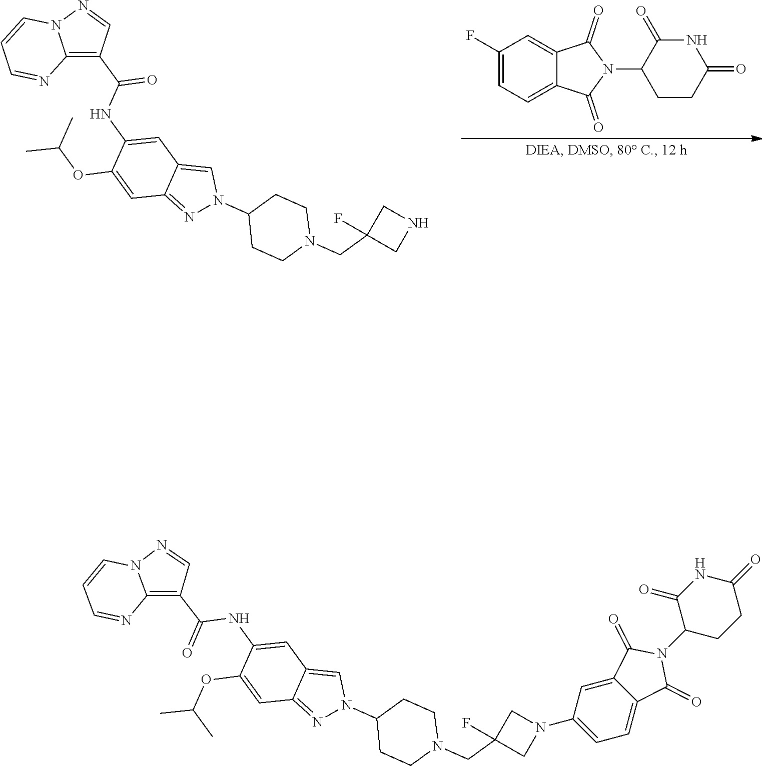

To a solution of N-[2-[1-[(3-fluoroazetidin-3-yl)methyl]-4-piperidyl]-6-isopropoxy-indazol-5-yl]pyrazolo[1,5-a]pyrimidine-3-carboxamide (100 mg, 197.4 μmol, 1 eq) was added 2-(2,6-dioxo-3-piperidyl)-5-fluoro-isoindoline-1,3-dione (109.05 mg, 394.81 μmol, 2 eq) and diisopropylethylamine (81.62 mg, 631.5 μmol, 110 uL, 3.20 eq) in dimethyl sulfoxide (10 mL). The mixture was stirred at 80° C. for 12 h. The reaction mixture was quenched by the addition of water (100 mL) at 0° C., and then extracted with dichloromethane (30 mL×3). The combined organic layers were washed with brine (30 mL×3), dried over anhydrous sodium sulfate, filtered, and concentrated under reduced pressure to give a residue. The residue was purified by preparative thin layer chromatography (silicon dioxide, dichloromethane:methanol=10:1) to afford N-[2-[1-[[1-[2-(2,6-dioxo-3-piperidyl)-1,3-dioxo-isoindolin-5-yl]-3-fluoro-azetidin-3-yl]methyl]-4-piperidyl]-6-isopropoxy-indazol-5-yl]pyrazolo[1,5-a]pyrimidine-3-carboxamide (66 mg, 79.6 mol, 40% yield, 92% purity) as a yellow solid. MS (ESI) m/z: 763.2 [M+H]⁺. ¹H NMR (400 MHz, DMSO-d₆) δ=10.59 (s, 1H), 8.92 (s, 1H), 8.84 (dd, J=1.6, 6.8 Hz, 1H), 8.81 (s, 1H), 8.71 (dd, J=1.6, 4.0 Hz, 1H), 8.16 (s, 1H), 7.87 (s, 1H), 7.70 (d, J=8.4 Hz, 1H), 7.09-7.03 (m, 2H), 6.87 (d, J=2.0 Hz, 1H), 6.67-6.60 (m, 1H), 4.96 (dd, J=5.2, 12.0 Hz, 1H), 4.83-4.74 (m, 1H), 4.47-4.07 (m, 5H), 3.30-3.11 (m, 2H), 3.07-2.96 (m, 2H), 2.94-2.71 (m, 4H), 2.61-2.42 (m, 2H), 2.38-2.12 (m, 5H), 1.56 (d, J=6.0 Hz, 6H).

The enantiomers of Example 265 could be separated by preparative SFC (column: DAICEL CHIRALPAK AD (250 mm×30 mm, 10 m); mobile phase: [IPA-acetonitrile]; B %: 70%-70%, 5; 70 min as additive) to yield Examples 266 and 267.

Example 266, (R)—N-(2-(1-((1-(2-(2,6-dioxopiperidin-3-yl)-1,3-dioxoisoindolin-5-yl)-3-fluoroazetidin-3-yl)methyl)piperidin-4-yl)-6-isopropoxy-2H-indazol-5-yl)pyrazolo[1,5-a]pyrimidine-3-carboxamide (126.9 mg, 41% yield, 97% purity) obtained as a yellow solid. MS (ESI) m/z: 763.3 [M+H]⁺. ¹H NMR (400 MHz, DMSO-d₆) δ=11.07 (s, 1H), 10.59 (s, 1H), 9.38 (dd, J=7.2, 1.6 Hz, 1H), 8.87 (dd, J=4.0, 1.6 Hz, 1H), 8.75 (s, 1H), 8.71 (s, 1H), 8.30 (s, 1H), 7.69 (d, J=8.4 Hz, 1H), 7.31-7.38 (m, 1H), 7.13 (s, 1H), 6.92 (d, J=2.0 Hz, 1H), 6.78 (dd, J 8.4, 2.0 Hz, 1H), 5.07 (dd, J=12.8, 5.2 Hz, 1H), 4.85 (dt, J=12.0, 6.0 Hz, 1H), 4.38 (s, 1H), 4.11-4.30 (m, 4H), 2.82-3.10 (m, 5H), 2.52-2.63 (m, 2H), 2.42 (t, J=9.6 Hz, 2H), 1.98-2.16 (m, 5H), 1.48 (d, J=6.0 Hz, 6H).

Example 267, (S)—N-(2-(1-((1-(2-(2,6-dioxopiperidin-3-yl)-1,3-dioxoisoindolin-5-yl)-3-fluoroazetidin-3-yl)methyl)piperidin-4-yl)-6-isopropoxy-2H-indazol-5-yl)pyrazolo[1,5-a]pyrimidine-3-carboxamide (120.8 mg, 40% yield, 99% purity) obtained as a yellow solid. MS (ESI) m/z: 763.2 [M+H]⁺. ¹H NMR (400 MHz, DMSO-d₆) δ=11.07 (s, 1H), 10.59 (s, 1H), 9.38 (dd, J=6.8, 1.6 Hz, 1H), 8.87 (dd, J=4.0, 1.6 Hz, 1H), 8.75 (s, 1H), 8.71 (s, 1H), 8.30 (s, 1H), 7.69 (d, J=8.4 Hz, 1H), 7.27-7.38 (m, 1H), 7.13 (s, 1H), 6.92 (d, J=2.0 Hz, 1H), 6.77 (dd, J 8.4, 2.0 Hz, 1H), 5.07 (dd, J=12.8, 5.2 Hz, 1H), 4.85 (dt, J=12.0, 6.0 Hz, 1H), 4.38 (s, 1 H), 4.12-4.30 (m, 4H), 2.83-3.11 (m, 5H), 2.52-2.64 (m, 2H), 2.36-2.46 (m, 2H), 1.99-2.18 (m, 5H), 1.47 (d, J=6.0 Hz, 6H).

### Preparation of Example 268: N-[6-(cyclopropylmethoxy)-2-[1-({1-[2-(2,6-dioxopiperidin-3-yl)-7-fluoro-3-oxo-2,3-dihydro-1H-isoindol-5-yl]-3-fluoroazetidin-3-yl}methyl)piperidin-4-yl]-2H-indazol-5-yl]pyrazolo[1,5-a]pyrimidine-3-carboxamide

### Step 1:

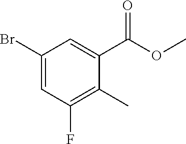

To a solution of 5-bromo-3-fluoro-2-methyl-benzoic acid (1.9 g, 8.2 mmol) in N,N-dimethylformamide (20 mL) was added potassium carbonate (2.25 g, 16.3 mmol) and iodomethane (3.47 g, 24.5 mmol, 1.52 mL). The mixture was stirred at 25° C. for 12 h, then filtered and the filtrate was concentrated in vacuo. The residue was purified by column chromatography (petroleum ether/ethyl acetate=20/1 to 3/1) to afford methyl 5-bromo-3-fluoro-2-methyl-benzoate (1.6 g, 79%) as a white solid.

### Step 2:

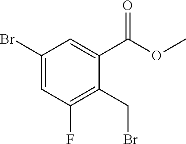

To a solution of methyl 5-bromo-3-fluoro-2-methyl-benzoate (1.4 g, 5.7 mmol) in CCl₄ (20 mL) was added N-Bromosuccinimide (1.01 g, 5.7 mmol) and benzoyl peroxide (686.3 mg, 2.8 mmol). The mixture was stirred at 90° C. for 12 h, then concentrated in vacuo. The residue was purified by column chromatography (petroleum ether/ethyl acetate=20/1 to 10/1) to afford methyl 5-bromo-2-(bromomethyl)-3-fluoro-benzoate (1.8 g, 97%) as a white solid.

### Step 3:

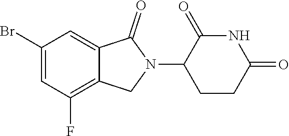 [SMILES: O=C1CCC(N2Cc3c(F)cc(Br)cc3C2=O)C(=O)N1]

To a solution of methyl 5-bromo-2-(bromomethyl)-3-fluoro-benzoate (1 g, 3.1 mmol) in N,N-dimethylformamide (10 mL) was added N,N-diisopropylethylamine (1.98 g, 15.3 mmol, 2.67 mL) and 3-aminopiperidine-2,6-dione hydrochloride (504.9 mg, 3.1 mmol). The mixture was stirred at 80° C. for 2 h, then filtered and the filtrate was concentrated in vacuo to afford 3-(6-bromo-4-fluoro-1-oxo-isoindolin-2-yl)piperidine-2,6-dione (0.6 g, crude) as a white solid. MS (ESI) m/z: 362.7 [M+23]⁺.

### Step 4:

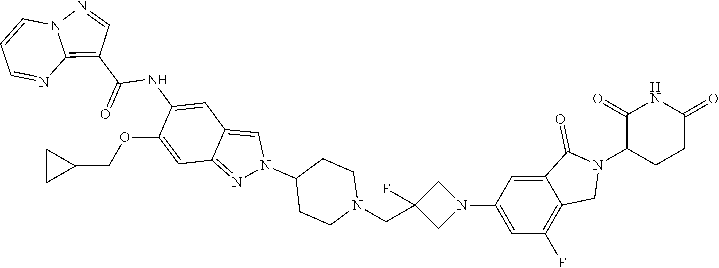 [SMILES: O=C1CCC(N2Cc3c(F)cc(N4CC(F)(CN5CCC(n6cc7cc(NC(=O)c8cnn9cccnc89)c(OCC8CC8)cc7n6)CC5)C4)cc3C2=O)C(=O)N1]

A mixture of N-(6-(cyclopropylmethoxy)-2-(1-((3-fluoroazetidin-3-yl)methyl)piperidin-4-yl)-2H-indazol-5-yl)pyrazolo[1,5-a]pyrimidine-3-carboxamide (350 mg, 674.9 μmol), 3-(6-bromo-4-fluoro-1-oxo-isoindolin-2-yl)piperidine-2,6-dione (230.23 mg, 674.9 μmol), cesium carbonate (439.80 mg, 1.4 mmol) and [1,3-bis(2,6-di-4-heptylphenyl)imidazol-2-ylidene](3-chloropyridyl)palladium(II) (224.60 mg, 270.0 μmol) in N,N-dimethylformamide (10 mL) was degassed and purged with nitrogen for 3 times, then the mixture was stirred at 80° C. for 1 hr under nitrogen atmosphere. To the reaction mixture was added water (10 mL) and the mixture was extracted with dichloromethane (3×20 mL). The combined organic phase was washed with brine (20 mL), dried over anhydrous sodium sulfate, filtered, and concentrated in vacuo. The crude product was purified by preparative HPLC (column: Phenomenex Synergi C18 150*25 mm*10 um; mobile phase: [water(formic acid)-acetonitrile]; B %: 14%-44%, 10 min) to afford N-(6-(cyclopropylmethoxy)-2-(1-((1-(2-(2,6-dioxopiperidin-3-yl)-7-fluoro-3-oxoisoindolin-5-yl)-3-fluoroazetidin-3-yl)methyl)piperidin-4-yl)-2H-indazol-5-yl)pyrazolo[1,5-a]pyrimidine-3-carboxamide (58.6 mg, 10%) as a white solid. MS (ESI) m/z: 724.4 [M+H]⁺; ¹H NMR (400 MHz, DMSO-d₆) δ 11.00 (s, 1H), 10.67 (s, 1H), 9.38 (dd, J=7.2, 1.6 Hz, 1H), 8.87 (dd, J=4.0, 1.6 Hz, 1H), 8.77 (s, 1H), 8.71 (s, 1H), 8.32 (s, 1H), 7.22-7.39 (m, 2H), 7.05 (s, 1H), 6.65-6.70 (m, 2H), 5.09 (dd, J=13.2, 5.2 Hz, 1H), 4.72 (s, 1H), 4.40-4.49 (m, 1H), 4.18-4.36 (m, 5H), 4.03 (d, J=6.8 Hz, 2H), 3.84-3.95 (m, 1H), 3.63 (s, 3H), 2.89 (d, J=13.2 Hz, 1H), 2.62 (s, 2H), 2.36-2.44 (m, 4H), 1.96-2.04 (m, 1H), 1.75 (s, 2H), 1.50 (s, 1H), 0.71 (d, J=7.6 Hz, 2H), 0.45 (d, J=4.8 Hz, 2H).

### Preparation of Example 269: N-[6-(cyclopropylmethoxy)-2-[1-({1-[2-(2,6-dioxopiperidin-3-yl)-6-fluoro-3-oxo-2,3-dihydro-1H-isoindol-5-yl]-3-fluoroazetidin-3-yl}methyl)piperidin-4-yl]-2H-indazol-5-yl]pyrazolo[1,5-a]pyrimidine-3-carboxamide

### Step 1:

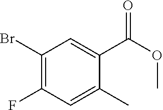

To a solution of 5-bromo-4-fluoro-2-methylbenzoic acid (2.1 g, 9.1 mmol) in N,N-dimethylformamide (20 mL) was added potassium carbonate (2.50 g, 18.1 mmol) and iodomethane (1.54 g, 10.9 mmol, 676.41 μL). The mixture was stirred at 20° C. for 12 h, then poured into water (100 mL). The aqueous phase was extracted with ethyl acetate (2×100 mL). The combined organic phase was washed with brine (2×80 mL), dried over anhydrous sodium sulfate, filtered, and concentrated in vacuum. The residue was purified by silica gel chromatography (0%-5% ethyl acetate in petroleum ether) to afford methyl 5-bromo-4-fluoro-2-methylbenzoate (2.18 g, 97%) as a white solid.

### Step 2:

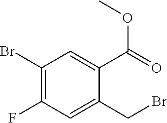

To a solution of methyl 5-bromo-4-fluoro-2-methylbenzoate (2.0 g, 8.1 mmol) in carbon tetrachloride (20 mL) was added 1-bromopyrrolidine-2,5-dione (1.44 g, 8.1 mmol) and benzoyl peroxide (196.09 mg, 809.5 umol) under nitrogen atmosphere. The mixture was stirred at 95° C. for 12 h under nitrogen atmosphere, then concentrated under reduced pressure. The residue was purified by silica gel chromatography (0%-1% ethyl acetate in petroleum ether) to afford methyl 5-bromo-2-(bromomethyl)-4-fluorobenzoate (1.73 g, 66%) as a colorless oil.

### Step 3:

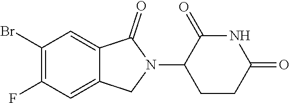 [SMILES: O=C1CCC(N2Cc3cc(F)c(Br)cc3C2=O)C(=O)N1]

To a solution of methyl 5-bromo-2-(bromomethyl)-4-fluorobenzoate (1.0 g, 3.1 mmol) in N,N-dimethylformamide (10 mL) was added N,N-diisopropylethylamine (1.59 g, 12.3 mmol, 2.14 mL) and 3-aminopiperidine-2,6-dione hydrochloride (504.94 mg, 3.1 mmol). The mixture was stirred at 80° C. for 20 h, then concentrated under reduced pressure. The crude product was triturated with water for 15 min, the suspension was filtered to afford 3-(6-bromo-5-fluoro-1-oxoisoindolin-2-yl)piperidine-2,6-dione (890 mg, 85%) as a black brown solid. MS (ESI) m/z: 363.0 [M+23]⁺.

### Step 4:

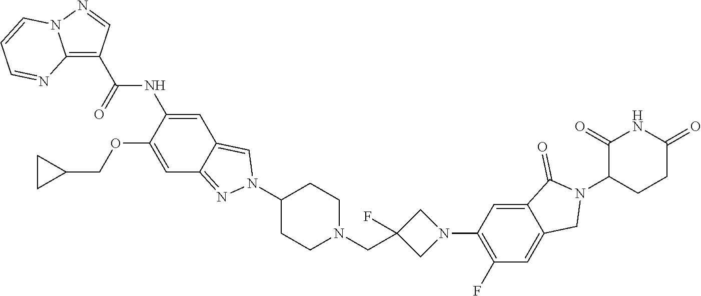 [SMILES: O=C1CCC(N2Cc3cc(F)c(N4CC(F)(CN5CCC(n6cc7cc(NC(=O)c8cnn9cccnc89)c(OCC8CC8)cc7n6)CC5)C4)cc3C2=O)C(=O)N1]

A mixture of N-(6-(cyclopropylmethoxy)-2-(1-((3-fluoroazetidin-3-yl)methyl)piperidin-4-yl)-2H-indazol-5-yl)pyrazolo[1,5-a]pyrimidine-3-carboxamide (300 mg, 578.5 umol), 3-(6-bromo-5-fluoro-1-oxoisoindolin-2-yl)piperidine-2,6-dione (197.34 mg, 578.5 umol), [1,3-Bis(2,6-Di-3-pentylphenyl)imidazol-2-ylidene](3-chloropyridyl)dichloropalladium(II) (91.83 mg, 115.7 umol) and cesium carbonate (376.97 mg, 1.2 mmol) in N,N-dimethylformamide (5 mL) was degassed and purged with nitrogen for 3 times, then the mixture was stirred at 80° C. for 3 h under nitrogen atmosphere. The mixture was filtered and concentrated under reduced pressure. The residue was purified by silica gel chromatography (0%-8% methanol in ethyl acetate), then further purified by semi-preparative reverse phase HPLC (column: Unisil 3-100 C18 Ultra 150*50 mm*3 um; mobile phase: [water (formic acid)-acetonitrile]; B %: 12%-42%, 10 min) to afford N-(6-(cyclopropylmethoxy)-2-(1-((1-(2-(2,6-dioxopiperidin-3-yl)-6-fluoro-3-oxoisoindolin-5-yl)-3-fluoroazetidin-3-yl)methyl)piperidin-4-yl)-2H-indazol-5-yl)pyrazolo[1,5-a]pyrimidine-3-carboxamide (40.7 mg, 8%) as an off-white solid. MS (ESI) m/z: 779.3 [M+H]⁺; ¹H NMR (400 MHz, DMSO-d₆) δ 10.97 (br s, 1H), 10.65 (s, 1H), 9.38 (dd, J=1.6, 7.2 Hz, 1H), 8.87 (dd, J=1.6, 4.0 Hz, 1H), 8.73 (d, J=10.8 Hz, 2H), 8.32 (s, 1H), 7.40 (d, J=11.6 Hz, 1H), 7.32 (dd, J=4.4, 7.2 Hz, 1H), 7.04 (s, 1H), 6.94 (d, J=8.4 Hz, 1H), 5.08 (dd, J=5.2, 13.2 Hz, 1H), 4.43-4.30 (m, 2H), 4.26-4.13 (m, 3H), 4.08 (br d, J=8.8 Hz, 1H), 4.03 (br d, J=7.2 Hz, 3H), 3.06 (br d, J=11.6 Hz, 2H), 3.00-2.87 (m, 3H), 2.61 (br d, J=2.8 Hz, 1H), 2.41-2.36 (m, 3H), 2.17-2.06 (m, 4H), 1.99 (td, J=5.2, 10.4 Hz, 1H), 1.55-1.45 (m, 1H), 0.74-0.68 (m, 2H), 0.49-0.44 (m, 2H).

### Preparation of Example 270: N-(6-(cyclopropylmethoxy)-2-(1-((1-(2-(2,6-dioxopiperidin-3-yl)-1,3-dioxoisoindolin-4-yl)-3-fluoroazetidin-3-yl)methyl)piperidin-4-yl)-2H-indazol-5-yl)pyrazolo[1,5-a]yrimidine-3-carboxamide

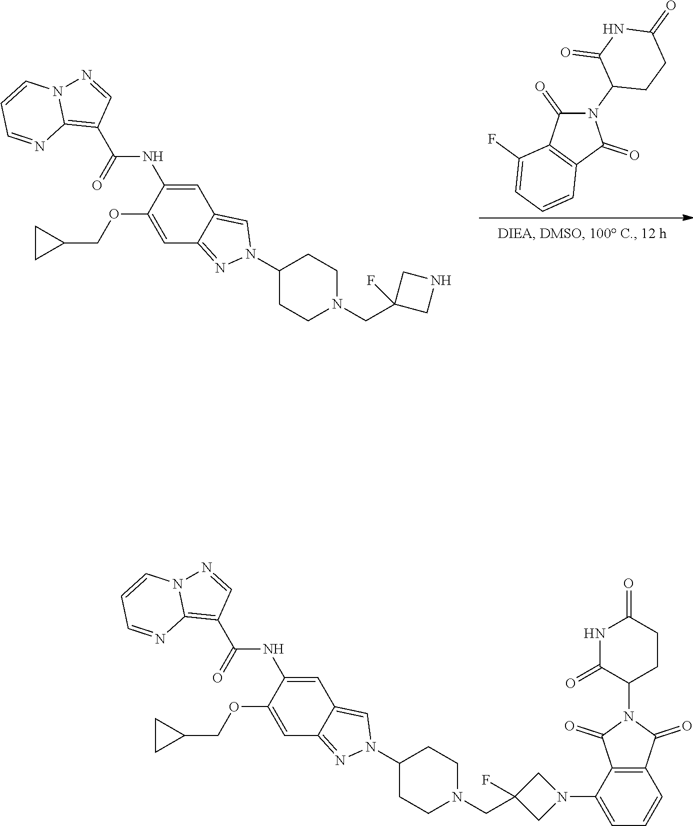

To a solution of N-(6-(cyclopropylmethoxy)-2-(1-((3-fluoroazetidin-3-yl)methyl)piperidin-4-yl)-2H-indazol-5-yl)pyrazolo[1,5-a]pyrimidine-3-carboxamide (0.1 g, 192.8 μmol, 1 eq) in dimethyl sulfoxide (2 mL) was added diisopropylethylamine (148.4 mg, 1.15 mmol, 0.2 mL, 5.95 eq) and 2-(2,6-dioxo-3-piperidyl)-4-fluoro-isoindoline-1,3-dione (55 mg, 199.12 μmol, 1.03 eq). The mixture was stirred at 100° C. for 12 h. The reaction mixture was quenched by the addition of water (10 mL) at 0° C., and then diluted with dichloromethane (30 mL) and extracted with dichloromethane (10 mL×3). The combined organic layers were washed with brine (10 mL×3), dried over anhydrous sodium sulfate, filtered, and concentrated under reduced pressure to give a residue. The residue was purified by column chromatography (silicon dioxide, eluted with dichloromethane:methanol=30/1 to 5/1) followed by re-crystallization from dichloromethane:methanol (10:1) 5M at 25° C. to afford N-(6-(cyclopropylmethoxy)-2-(1-((1-(2-(2,6-dioxopiperidin-3-yl)-1,3-dioxoisoindolin-4-yl)-3-fluoroazetidin-3-yl)methyl)piperidin-4-yl)-2H-indazol-5-yl)pyrazolo[1,5-a]pyrimidine-3-carboxamide (110.9 mg, 135.98 μmol, 70% yield, 95% purity) as a yellow solid. MS (ESI) m/z: 775.3[M+H]⁺. ¹H NMR (400 MHz, DMSO-d₆) δ=11.08 (s, 1H), 10.64 (s, 1H), 9.37 (dd, J=1.6, 7.2 Hz, 1H), 8.86 (dd, J=1.2, 4.0 Hz, 1H), 8.72 (d, J=11.2 Hz, 2H), 8.31 (s, 1H), 7.61 (dd, J=7.2, 8.4 Hz, 1H), 7.31 (dd, J=4.4, 6.8 Hz, 1H), 7.19 (d, J=6.8 Hz, 1H), 7.03 (s, 1H), 6.89 (d, J=8.8 Hz, 1H), 5.08 (dd, J=5.2, 12.8 Hz, 1H), 4.48-4.20 (m, 5H), 4.02 (d, J=6.8 Hz, 2H), 3.30 (s, 1H), 3.05 (m, 2H), 2.97 (s, 1H), 2.94-2.81 (m, 2H), 2.64-2.53 (m, 2H), 2.39 (m, 2H), 2.14-2.03 (m, 4H), 1.56-1.43 (m, 1H), 0.71 (m, 2H), 0.46 (m, 2H).

### Preparation of Example 271: N-(2-(1-((1-(2-(2,6-dioxopiperidin-3-yl)-1,3-dioxoisoindolin-4-yl)-3-fluoroazetidin-3-yl)methyl)piperidin-4-yl)-6-isopropoxy-2H-indazol-5-yl)pyrazolo[1,5-a]pyrimidine-3-carboxamide

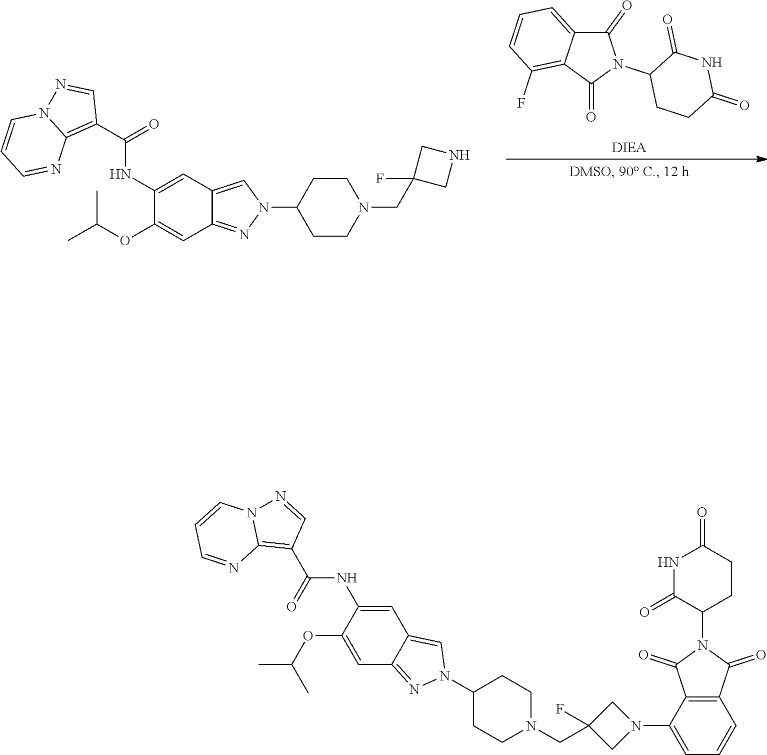 [SMILES: CC(C)Oc1cc2nn(C3CCN(CC4(F)CNC4)CC3)cc2cc1NC(=O)c1cnn2cccnc12 ; O=C1CCC(N2C(=O)c3cccc(F)c3C2=O)C(=O)N1 ; "DIEA" ; "DMSO, 90° C., 12 h" ; CC(C)Oc1cc2nn(C3CCN(CC4(F)CN(c5cccc6c5C(=O)N(C5CCC(=O)NC5=O)C6=O)C4)CC3)cc2cc1NC(=O)c1cnn2cccnc12]

To a solution of N-[2-[1-[(3-fluoroazetidin-3-yl)methyl]-4-piperidyl]-6-isopropoxy-indazol-5-yl]pyrazolo[1,5-a]pyrimidine-3-carboxamide (0.08 g, 157.92 μmol, 1 eq) in dimethyl sulfoxide (2 mL) was added N,N-diisopropylethylamine (102.05 mg, 789.62 μmol, 137.54 μL, 5 eq). The mixture was stirred at 90° C. for 12 h. The reaction mixture was poured into water (30 mL) at 0° C. and extracted with dichloromethane 30 mL (30 mL×3). The combined organic layers were washed with brine 50 mL (50 mL×3), dried over sodium sulfate, filtered, and concentrated under reduced pressure to give a residue. The residue was purified by preparative thin-layer chromatography (Silicon dioxide, dichloromethane/methyl alcohol=15/1) to afford N-[2-[1-[[1-[2-(2,6-dioxo-3-piperidyl)-1,3-dioxo-isoindolin-4-yl]-3-fluoro-azetidin-3-yl]methyl]-4-piperidyl]-6-isopropoxy-indazol-5-yl]pyrazolo[1,5-a]pyrimidine-3-carboxamide (80.5 mg, 101.3 mol, 64% yield, 96% purity) as a yellow solid. MS (ESI) m/z: 763.6 [M+H]⁺. ¹H NMR (400 MHz, DMSO-d₆) δ=11.14-11.00 (m, 1H), 10.59 (s, 1H), 9.38 (dd, J=1.6, 7.2 Hz, 1H), 8.87 (dd, J=1.6, 4.0 Hz, 1H), 8.72 (d, J=14.4 Hz, 2H), 8.32 (s, 1H), 7.62 (dd, J=7.2, 8.4 Hz, 1H), 7.34 (dd, J=4.0, 6.8 Hz, 1H), 7.19 (d, J=7.2 Hz, 1H), 7.13 (s, 1H), 6.90 (d, J=8.0 Hz, 1H), 5.08 (dd, J=5.2, 12.4 Hz, 1H), 4.85 (t, J=6.0 Hz, 1H), 4.49-4.20 (m, 5H), 3.29 (s, 1H), 3.11-3.01 (m, 2H), 2.98 (s, 1H), 2.92 (s, 1H), 2.70-2.55 (m, 2H), 2.44-2.36 (m, 2H), 2.15-1.99 (m, 5H), 1.48 (d, J=6.0 Hz, 6H).

### Preparation of Example 272: N-(2-(1-((1-(2-(2,6-dioxopiperidin-3-yl)-1,3-dioxoisoindolin-5-yl)-3-fluoroazetidin-3-yl)methyl)piperidin-4-yl)-6-fluoro-2H-indazol-5-yl)pyrazolo[1,5-a]pyrimidine-3-carboxamide

### Step 1:

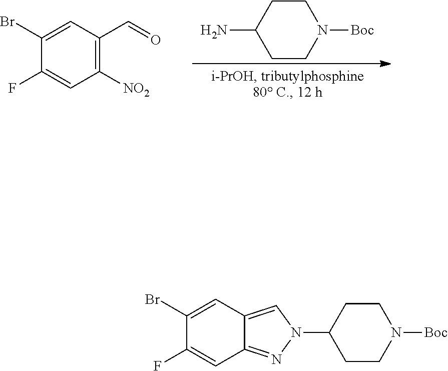

To a solution of 5-bromo-4-fluoro-2-nitrobenzaldehyde (900 mg, 3.63 mmol, 1 eq) in isopropyl alcohol (10 mL) was added tert-butyl 4-aminopiperidine-1-carboxylate (726.80 mg, 3.63 mmol, 1 eq). The reaction mixture was stirred at 80° C. for 1 h, followed by the addition of tributylphosphine (2.20 g, 10.89 mmol, 2.69 mL, 3 eq). The reaction was stirred at 80° C. for 11 h. The reaction mixture was concentrated in vacuo to give the crude product. The residue was purified by column chromatography (silicon dioxide, eluted with petroleum ether/ethyl acetate=20/1 to 3/1) to afford tert-butyl 4-(5-bromo-6-fluoro-2H-indazol-2-yl)piperidine-1-carboxylate (1.1 g, 2.76 mmol, 76% yield), obtained as a white solid. MS (ESI) m/z: 344.0 [M-tBu]⁺. ¹H NMR (400 MHz, CDCl₃) δ=7.92 (s, 1H), 7.89 (d, J=6.8 Hz, 1H), 7.39 (d, J=9.2 Hz, 1H), 4.53 (tt, J=11.6, 4.0 Hz, 1H), 4.32 (s, 2H), 2.94 (t, J=12.0 Hz, 2H), 2.24 (d, J=10.0 Hz, 2H), 2.07 (qd, J=12.4, 4.0 Hz, 2H), 1.49 (s, 9H).

### Step 2:

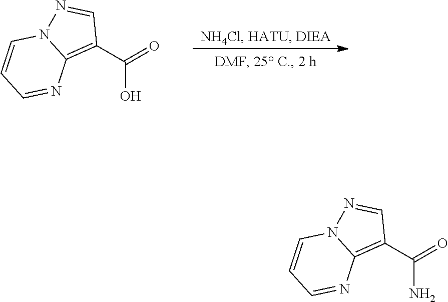 [SMILES: O=C(O)c1cnn2cccnc12 ; "DMF, 25° C., 2 h" ; "NH4Cl, HATU, DIEA" ; NC(=O)c1cnn2cccnc12]

To a solution of pyrazolo[1,5-a]pyrimidine-3-carboxylic acid (2 g, 12.26 mmol, 1 eq), ammonium chloride (1.97 g, 36.78 mmol, 3 eq) in DMF (50 mL) was added HATU (6.99 g, 18.39 mmol, 1.5 eq) and diisopropylethylamine (4.75 g, 36.78 mmol, 6.41 mL, 3 eq). The mixture was stirred at 25° C. for 2 h. The reaction mixture was concentrated in vacuo to give the crude product. The residue was purified by column chromatography (silicon dioxide, eluted with dichloromethane/methanol=30/1 to 10:1) to afford pyrazolo[1,5-a]pyrimidine-3-carboxamide (1.1 g, 6.78 mmol, 55% yield) as a white solid. MS (ESI) m/z: 164.0 [M+H]⁺. ¹H NMR (400 MHz, DMSO-d₆) 9.30 (dd, J=7.2, 1.6 Hz, 1H), 8.79 (dd, J=4.0, 1.6 Hz, 1H), 8.56 (s, 1H), 7.48 (d, J=13.2 Hz, 2H), 7.26 (dd, J=7.2, 4.4 Hz, 1H).

### Step 3:

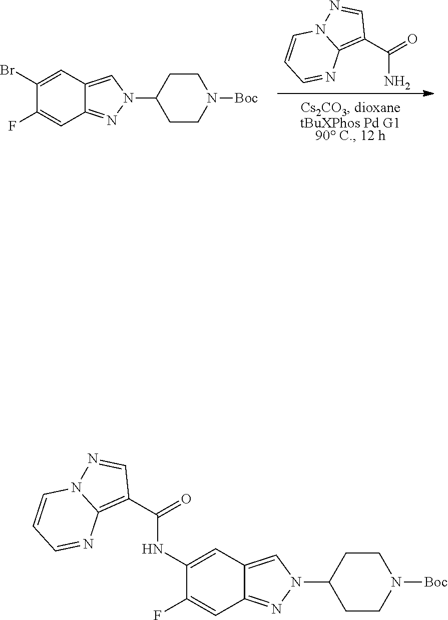 [SMILES: CC(C)(C)OC(=O)N1CCC(n2cc3cc(Br)c(F)cc3n2)CC1 ; NC(=O)c1cnn2cccnc12 ; "Cs2CO3, dioxanetBuXPhos Pd G190° C., 12 h" ; CC(C)(C)OC(=O)N1CCC(n2cc3cc(NC(=O)c4cnn5cccnc45)c(F)cc3n2)CC1]

A mixture of tert-butyl 4-(5-bromo-6-fluoro-2H-indazol-2-yl)piperidine-1-carboxylate (700 mg, 1.76 mmol, 1 eq), pyrazolo[1,5-a]pyrimidine-3-carboxamide (284.99 mg, 1.76 mmol, 1 eq), tBuXPhos Pd G1 (161.79 mg, 175.76 μmol, 0.1 eq), cesium carbonate (1.15 g, 3.52 mmol, 2 eq) in dioxane (30 mL) was degassed and purged with nitrogen atmosphere in 3 cycles, and then the mixture was stirred at 90° C. for 12 h under a nitrogen atmosphere. The reaction mixture was concentrated in vacuo to give the crude product. The residue was purified by column chromatography (silicon dioxide, eluted with dichloromethane/methanol=30/1 to 10/1) to afford tert-butyl 4-(6-fluoro-5-(pyrazolo[1,5-a]pyrimidine-3-carboxamido)-2H-indazol-2-yl)piperidine-1-carboxylate (840 mg, 1.75 mmol, 99% yield) as a white solid. MS (ESI) m/z: 480.2 [M+H]⁺.

### Step 4:

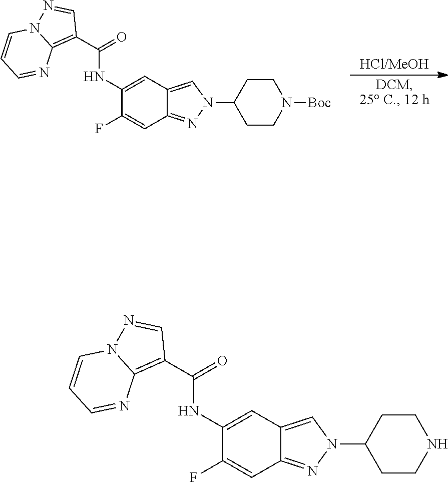 [SMILES: CC(C)(C)OC(=O)N1CCC(n2cc3cc(NC(=O)c4cnn5cccnc45)c(F)cc3n2)CC1 ; "DCM,25° C., 12 h" ; "HCl/MeOH" ; O=C(Nc1cc2cn(C3CCNCC3)nc2cc1F)c1cnn2cccnc12]

To a solution of tert-butyl 4-(6-fluoro-5-(pyrazolo[1,5-a]pyrimidine-3-carboxamido)-2H-indazol-2-yl)piperidine-1-carboxylate (840 mg, 1.75 mmol, 1 eq) in dichloromethane (10 mL) was added hydrogen chloride/methanol (4 M, 20.00 mL, 45.67 eq). The mixture was stirred at 25° C. for 12 h. The reaction mixture was concentrated in vacuo to give a residue which was resuspended in dichloromethane (30 mL) and the pH adjusted to 8 with the addition of ammonium hydroxide. The combined organic phases were dried over anhydrous sodium sulfate, filtered and concentrated under reduced pressure to give a residue. The crude product was purified by preparative HPLC (column: Phenomenex luna C18 250×50 mm×10 m; mobile phase: [water(10 mM ammonium bicarbonate)-acetonitrile]; B %: 40%-70% acetonitrile, 20 min as additive) to yield N-(6-fluoro-2-(piperidin-4-yl)-2H-indazol-5-yl)pyrazolo[1,5-a]pyrimidine-3-carboxamide (450 mg, 1.19 mmol, 67% yield) as a white solid. MS (ESI) m/z: 380.1 [M+H]⁺.

### Step 5:

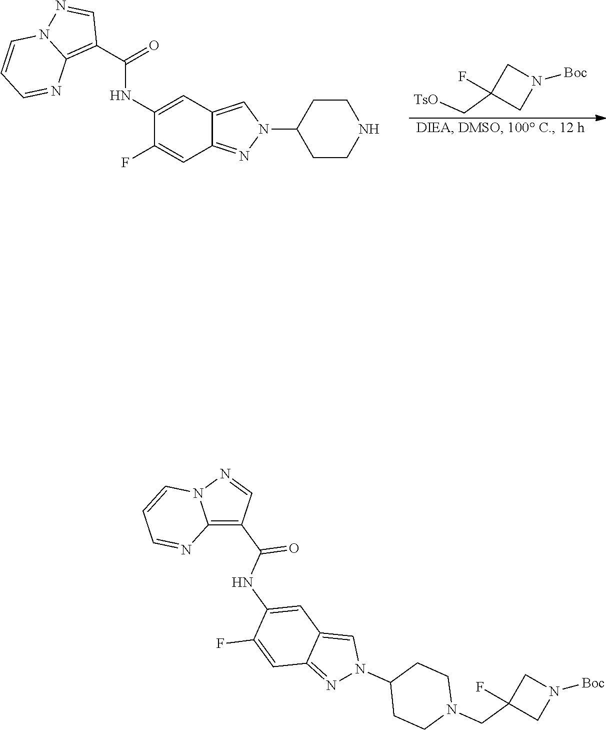

To a solution of N-(6-fluoro-2-(piperidin-4-yl)-2H-indazol-5-yl)pyrazolo[1,5-a]pyrimidine-3-carboxamide (300 mg, 790.74 μmol, 1 eq) in dimethyl sulfoxide (2 mL) was added diisopropylethylamine (510.99 mg, 3.95 mmol, 688.67 μL, 5 eq) and tert-butyl 3-fluoro-3-((tosyloxy)methyl)azetidine-1-carboxylate (568.41 mg, 1.58 mmol, 2 eq). The mixture was stirred at 100° C. for 12 h. The reaction mixture was diluted with water (10 mL) and the mixture was extracted with dichloromethane (20 mL×3). The combined organic phase was washed with brine (20 mL), dried over anhydrous sodium sulfate, filtered, and concentrated in vacuo. The residue was purified by column chromatography (silicon dioxide, eluted with dichloromethane/methanol=30/1 to 10:1) to afford tert-butyl 3-fluoro-3-((4-(6-fluoro-5-(pyrazolo[1,5-a]pyrimidine-3-carboxamido)-2H-indazol-2-yl)piperidin-1-yl)methyl)azetidine-1-carboxylate (300 mg, 529.47 mol, 66% yield) as a white solid. MS (ESI) m/z: 567.5 [M+H]⁺.

### Step 6:

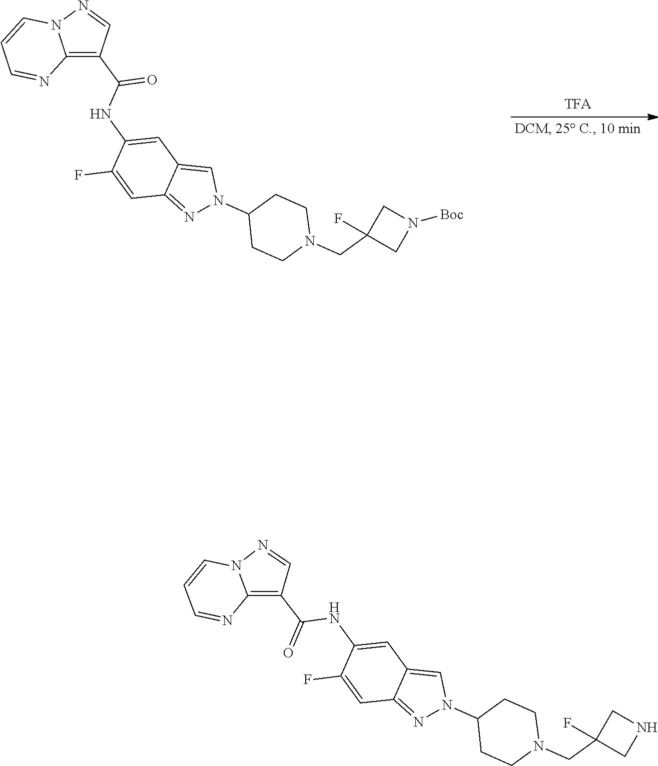 [SMILES: CC(C)(C)OC(=O)N1CC(F)(CN2CCC(n3cc4cc(NC(=O)c5cnn6cccnc56)c(F)cc4n3)CC2)C1 ; "DCM, 25° C., 10 min" ; "TFA" ; O=C(Nc1cc2cn(C3CCN(CC4(F)CNC4)CC3)nc2cc1F)c1cnn2cccnc12]

To a solution of tert-butyl 3-fluoro-3-((4-(6-fluoro-5-(pyrazolo[1,5-a]pyrimidine-3-carboxamido)-2H-indazol-2-yl)piperidin-1-yl)methyl)azetidine-1-carboxylate (200 mg, 352.98 μmol, 1 eq) in dichloromethane (10 mL) was added trifluoroacetic acid (7.70 g, 67.53 mmol, 5 mL, 191.31 eq). The mixture was stirred at 25° C. for 10 min. The reaction mixture was concentrated under reduced pressure to yield N-(6-fluoro-2-(1-((3-fluoroazetidin-3-yl)methyl)piperidin-4-yl)-2H-indazol-5-yl)pyrazolo[1,5-a]pyrimidine-3-carboxamide (160 mg, crude), obtained as a white solid. MS (ESI) m/z: 467.2 [M+H]⁺.

### Step 7:

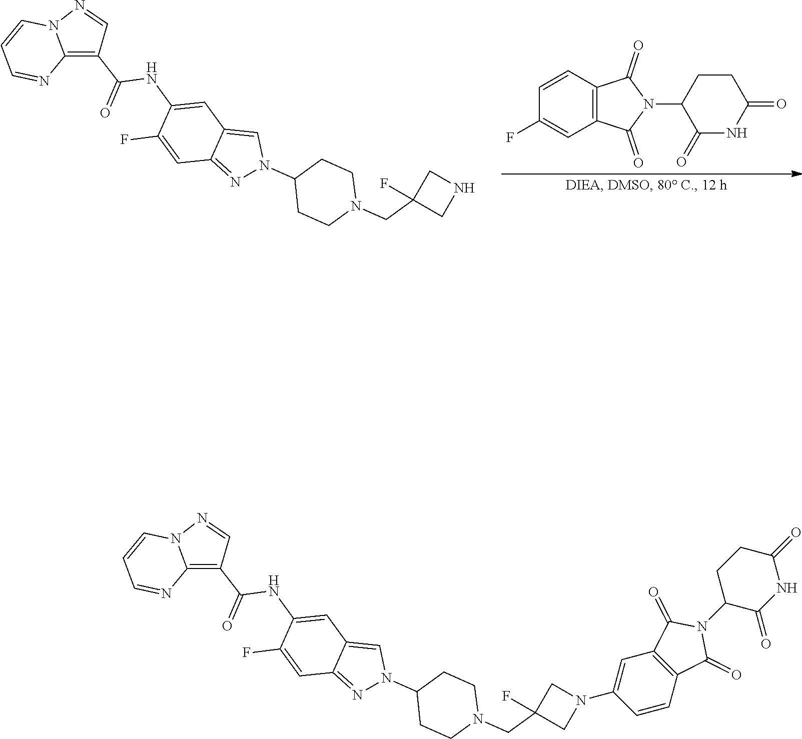

To a solution of N-(6-fluoro-2-(1-((3-fluoroazetidin-3-yl)methyl)piperidin-4-yl)-2H-indazol-5-yl)pyrazolo[1,5-a]pyrimidine-3-carboxamide (160 mg, 342.99 μmol, 1 eq) in dimethyl sulfoxide (2 mL) was added diisopropylethylamine (221.65 mg, 1.71 mmol, 298.71 μL, 5 eq) and 2-(2,6-dioxopiperidin-3-yl)-5-fluoroisoindoline-1,3-dione (170.53 mg, 617.38 μmol, 1.8 eq). The mixture was stirred at 80° C. for 12 h. To the reaction mixture was added water (10 mL) and the mixture was extracted with dichloromethane (20 mL×3). The combined organic phase was washed with brine (20 mL), dried over anhydrous sodium sulfate, filtered, and concentrated in vacuo. The crude product was purified by preparative HPLC (column: Phenomenex luna C18 150×25 mm×10 m; mobile phase: [water(0.225% formic acid)-acetonitrile]; B %: 15%-45%, 10 min as additive) to yield N-(2-(1-((1-(2-(2,6-dioxopiperidin-3-yl)-1,3-dioxoisoindolin-5-yl)-3-fluoroazetidin-3-yl)methyl)piperidin-4-yl)-6-fluoro-2H-indazol-5-yl)pyrazolo[1,5-a]pyrimidine-3-carboxamide (122.4 mg, 164.28 μmol, 47% yield, 97% purity) as a yellow solid. MS (ESI) m/z: 723.4 [M+H]⁺. ¹H NMR (400 MHz, DMSO-d₆) δ=11.09 (s, 1H), 10.20 (d, J=3.6 Hz, 1H), 9.41 (dd, J=7.2, 1.6 Hz, 1H), 8.94 (dd, J=4.4, 1.6 Hz, 1H), 8.75 (s, 1H), 8.68 (d, J=8.0 Hz, 1H), 8.51 (s, 1H), 7.69 (d, J=8.4 Hz, 1H), 7.55 (d, J=12.4 Hz, 1H), 7.32-7.41 (m, 1H), 6.92 (d, J=1.6 Hz, 1H), 6.78 (dd, J=8.4, 2.0 Hz, 1H), 5.07 (dd, J=12.8, 5.6 Hz, 1H), 4.46 (dt, J=10.4, 5.2 Hz, 1H), 4.11-4.30 (m, 4H), 3.07 (d, J=11.6 Hz, 2H), 2.99 (s, 1H), 2.93 (s, 1H), 2.82-2.90 (m, 1H), 2.54-2.62 (m, 2H), 2.39-2.46 (m, 2H), 2.06-2.17 (m, 4H), 1.98-2.04 (m, 1H).

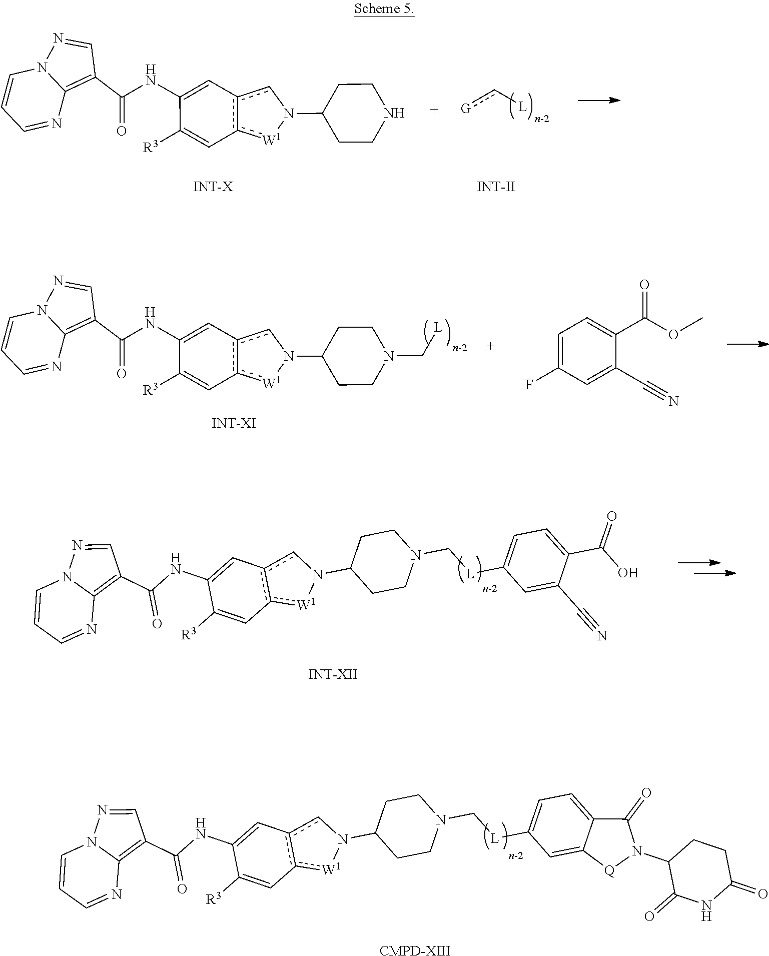

A compound of formula INTAX, wherein R³ is an ether, amine, or cycloalkyl group and W¹ is optionally N or C═O, may be reacted with a reagent INT-II (commercially available or readily prepared using standard reaction techniques known to one skilled in the art) under reductive amination or alkylation conditions to produce a compound of formula INT-XI, wherein L and n are as defined herein. Compounds of formula INT-XI can be furnished from N-alkylation where G is an appropriate leaving group (e.g., OMs, OTs, Cl, etc.) or through reductive amination where G is an aldehyde. When G is a leaving group, suitable reaction conditions are those for an alkylation reaction, e.g., diisopropylethylamine, potassium iodide, DMSO or acetonitrile, 80° C. When G is an aldehyde, suitable reaction conditions are those for a reductive amination reaction, e.g., sodium cyanoborohydride, methanol, dichloromethane, acetic acid, room temperature. Compounds of formula INT-XI can then be transformed to compounds of formula CNIPD-XIII in two steps involving nitrile reduction and condensation with 3-aminopiperidine-2,6-dione under standard reaction conditions known to one skilled in the art.

### Preparation of Example 273: N-(2-((1r,4r)-4-(4-(2-(2,6-dioxopiperidin-3-yl)-1-oxoisoindolin-5-yl)piperazin-1-yl)cyclohexyl)-6-isopropoxy-1-oxoisoindolin-5-yl)pyrazolo[1,5-a]pyrimidine-3-carboxamide

### Step 1:

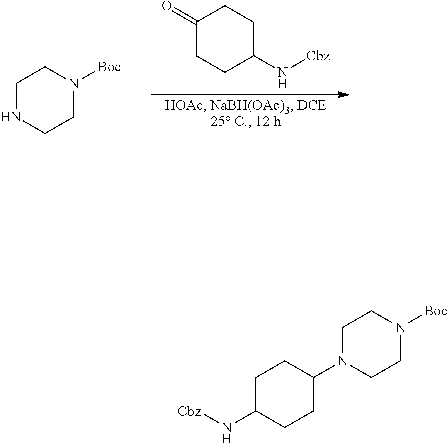

To a solution of benzyl (4-oxocyclohexyl)carbamate (30 g, 121.32 mmol, 1 eq) and tert-butyl piperazine-1-carboxylate (27.02 g, 121.32 mmol, 1 eq, hydrochloride) in 1,2-dichloroethane (300 mL) was added acetic acid (10.93 g, 181.97 mmol, 10.41 mL, 1.5 eq). The reaction mixture was allowed to stir at 25° C. for 1 h. Sodium triacetoxyborohydride (77.13 g, 363.95 mmol, 3 eq) was added to the above reaction mixture and stirred at 25° C. for 11 h. The reaction mixture was quenched with water (400 mL), adjusted to pH=8 with a saturated aqueous solution of sodium bicarbonate at 0° C., extracted with dichloromethane (200 mL×3). The combined organic layers were washed with brine (200 mL), dried over anhydrous sodium sulfate, filtered, and concentrated to give a residue. The residue was purified by silica gel column chromatography (eluted with petroleum ether/ethyl acetate=5/1 to 0/1) to afford tert-butyl 4-(4-(((benzyloxy)carbonyl)amino)cyclohexyl)piperazine-1-carboxylate (40 g, 95.8 mmol, 79% yield) as a white solid. MS (ESI) m/z: 418.2 [M+H]⁺.

### Step 2:

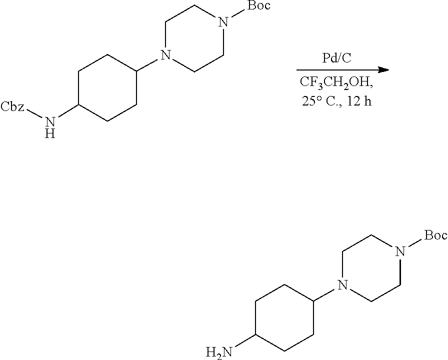 [SMILES: CC(C)(C)OC(=O)N1CCN(C2CCC(NC(=O)OCc3ccccc3)CC2)CC1 ; CC(C)(C)OC(=O)N1CCN(C2CCC(N)CC2)CC1]

To a solution of tert-butyl 4-(4-(((benzyloxy)carbonyl)amino)cyclohexyl)piperazine-1-carboxylate (36 g, 86.22 mmol, 1 eq) in 2,2,2-trifluoroethanol (400 mL) was added palladium on carbon (3.6 g, 3.38 mmol, 10% purity, 0.0392 eq) under an atmosphere of nitrogen. The suspension was degassed under vacuo and purged with an atmosphere of hydrogen over several cycles. The mixture was stirred under a balloon of hydrogen gas (15 psi) at 25° C. for 12 h. The reaction mixture was filtered, and the filtrate was concentrated under reduced pressure to yield tert-butyl 4-(4-aminocyclohexyl)piperazine-1-carboxylate (24 g, 84.68 mmol, 98% yield) as a yellow oil which was used into the next step without further purification.

### Step 3:

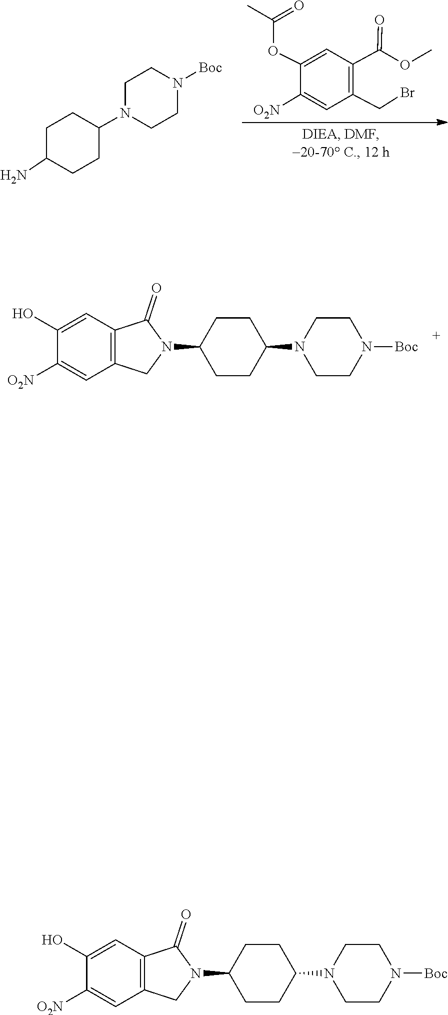 [SMILES: CC(C)(C)OC(=O)N1CCN(C2CCC(N)CC2)CC1 ; COC(=O)c1cc(OC(C)=O)c([N+](=O)[O-])cc1CBr ; CC(C)(C)OC(=O)N1CCN([C@H]2CC[C@@H](N3Cc4cc([N+](=O)[O-])c(O)cc4C3=O)CC2)CC1 ; CC(C)(C)OC(=O)N1CCN([C@H]2CC[C@H](N3Cc4cc([N+](=O)[O-])c(O)cc4C3=O)CC2)CC1]

To a solution of tert-butyl 4-(4-aminocyclohexyl)piperazine-1-carboxylate (5.25 g, 18.52 mmol, 2.05 eq) and diisopropylethylamine (3.90 g, 30.14 mmol, 5.25 mL, 3.34 eq) in N,N-dimethylformamide (40 mL) was added the solution of methyl 5-acetoxy-2-(bromomethyl)-4-nitrobenzoate (3 g, 9.03 mmol, 1 eq) in N,N-dimethylformamide (20 mL) at −20° C. The mixture was stirred at 0° C. for 1 h followed by at 70° C. for 11 h. The reaction mixture was concentrated under reduced pressure to give a residue. The residue was purified by silica gel column chromatography (eluted with dichloromethane:methanol=100:1 to 20:1) to afford both the cis and trans isomers. Cis: tert-butyl 4-((1s,4s)-4-(6-hydroxy-5-nitro-1-oxoisoindolin-2-yl)cyclohexyl)piperazine-1-carboxylate (0.6 g, 1.25 mmol, 14% yield, 96% purity, yellow solid); MS (ESI) m/z: 461.2 [M+H]⁺. ¹H NMR (400 MHz, methanol-d₄) δ=8.22 (s, 1H), 7.44 (s, 1H), 4.54 (s, 2H), 4.32-4.22 (m, 1H), 3.48 (t, J=3.6 Hz, 4H), 2.49 (br t, J=4.8 Hz, 4H), 2.26 (br s, 1H), 2.21-1.97 (m, 4H), 1.70-1.54 (m, 4H), 1.47 (s, 9H).

Trans: tert-butyl 4-((1r,4r)-4-(6-hydroxy-5-nitro-1-oxoisoindolin-2-yl)cyclohexyl)piperazine-1-carboxylate (2.3 g, 4.99 mmol, 55% yield, 100% purity, white solid); MS (ESI) m/z: 461.2 [M+H]⁺. ¹H NMR (400 MHz, methanol-d₄) δ=8.21 (s, 1H), 7.44 (s, 1H), 4.49 (s, 2H), 4.20-4.09 (m, 1H), 3.61-3.48 (m, 4H), 2.86 (br t, J=4.4 Hz, 4H), 2.81-2.71 (m, 1H), 2.15 (br d, J=11.6 Hz, 2H), 2.05-1.96 (m, 2H), 1.84-1.70 (m, 2H), 1.67-1.55 (m, 2H), 1.47 (s, 9H)

### Step 4:

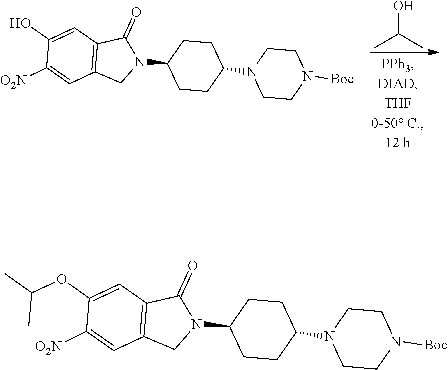

To a solution of tert-butyl 4-((1r,4r)-4-(6-hydroxy-5-nitro-1-oxoisoindolin-2-yl)cyclohexyl)piperazine-1-carboxylate (2 g, 4.34 mmol, 1 eq), propan-2-ol (800 mg, 13.31 mmol, 1.02 mL, 3.07 eq) and triphenylphosphine (4 g, 15.25 mmol, 3.51 eq) in tetrahydrofuran (800 mL) was added diisopropyl azodicarboxylate (3.12 g, 15.43 mmol, 3 mL, 3.55 eq) at 0° C. The mixture was stirred at 50° C. for 12 h. The reaction mixture was concentrated under reduced pressure to give a residue, and then diluted with ethyl acetate (400 mL). The combined organic layers were washed with brine (100 mL×3), dried over anhydrous sodium sulfate, filtered, and concentrated under reduced pressure to give a residue. The residue was purified by silica gel column chromatography (eluted with petroleum ether/ethyl acetate=1/1 to 0/1) to afford tert-butyl 4-((1r,4r)-4-(6-isopropoxy-5-nitro-1-oxoisoindolin-2-yl)cyclohexyl)piperazine-1-carboxylate (1.5 g, 2.98 mmol, 69% yield) as a yellow solid. MS (ESI) m/z: 473.3 [M+H]⁺. ¹H NMR: (400 MHz, DMSO-d₆) δ=8.05 (s, 1H), 7.55 (s, 1H), 5.01-4.87 (m, 1H), 4.42 (s, 2H), 4.05-3.89 (m, 1H), 3.29 (br s, 4H), 2.47-2.42 (m, 4H), 2.37-2.28 (m, 1H), 1.90-1.77 (m, 4H), 1.66-1.53 (m, 2H), 1.45-1.34 (m, 11H), 1.28 (d, J=6.0 Hz, 6H).

### Step 5:

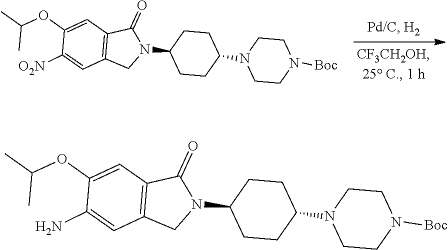 [SMILES: CC(C)Oc1cc2c(cc1[N+](=O)[O-])CN([C@H]1CC[C@H](N3CCN(C(=O)OC(C)(C)C)CC3)CC1)C2=O ; CC(C)Oc1cc2c(cc1N)CN([C@H]1CC[C@H](N3CCN(C(=O)OC(C)(C)C)CC3)CC1)C2=O]

To a solution of tert-butyl 4-((1r,4r)-4-(6-isopropoxy-5-nitro-1-oxoisoindolin-2-yl)cyclohexyl)piperazine-1-carboxylate (1.5 g, 2.98 mmol, 1 eq) in 2,2,2-trifluoroethanol (150 mL) was added palladium on carbon (500 mg, 469.84 μmol, 10% purity, 0.157 eq) under atmosphere of nitrogen. The suspension was degassed under vacuo and purged with an atmosphere of hydrogen over several cycles. The mixture was stirred under a balloon (15 psi) atmosphere of hydrogen at 25° C. for 1 h. The reaction mixture was filtered and concentrated in vacuo to give a residue. The crude product tert-butyl 4-((1r,4r)-4-(5-amino-6-isopropoxy-1-oxoisoindolin-2-yl)cyclohexyl)piperazine-1-carboxylate (1.4 g, 2.96 mmol, 99% yield) was obtained as a white solid and used in the next step without further purification. MS (ESI) m/z: 473.3 [M+H]⁺. ¹H NMR (400 MHz, DMSO-d₆) δ=6.96 (s, 1H), 6.71 (s, 1H), 5.28 (s, 2H), 4.62-4.50 (m, 1H), 4.16 (s, 2H), 3.95-3.81 (m, 1H), 3.28 (br s, 4H), 2.44 (br s, 4H), 2.36-2.23 (m, 1H), 1.84 (br d, J=11.2 Hz, 2H), 1.73 (br d, J=10.4 Hz, 2H), 1.60-1.47 (m, 2H), 1.45-1.32 (m, 11H), 1.27 (d, J=6.0 Hz, 6H).

### Step 6:

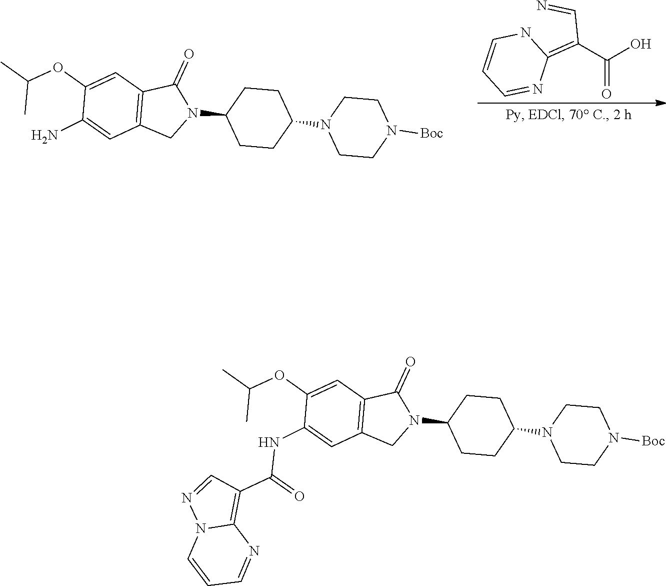 [SMILES: CC(C)Oc1cc2c(cc1N)CN([C@H]1CC[C@H](N3CCN(C(=O)OC(C)(C)C)CC3)CC1)C2=O ; N=C/C(C(=O)O)=C1/N=CC=CN1 ; CC(C)Oc1cc2c(cc1NC(=O)c1cnn3cccnc13)CN([C@H]1CC[C@H](N3CCN(C(=O)OC(C)(C)C)CC3)CC1)C2=O]

To a solution of tert-butyl 4-((1r,4r)-4-(5-amino-6-isopropoxy-1-oxoisoindolin-2-yl)cyclohexyl)piperazine-1-carboxylate (1.4 g, 2.96 mmol, 1 eq) and pyrazolo[1,5-a]pyrimidine-3-carboxylic acid (1 g, 6.13 mmol, 2.07 eq) in pyridine (15 mL) was added EDCI (2.5 g, 13.04 mmol, 4.40 eq). The mixture was stirred at 70° C. for 2 h. The reaction mixture was concentrated under reduced pressure to give a residue. The residue was purified by silica gel column chromatography (dichloromethane:methanol=100/1 to 10/1). Compound tert-butyl 4-((1r,4r)-4-(6-isopropoxy-1-oxo-5-(pyrazolo[1,5-a]pyrimidine-3-carboxamido)isoindolin-2-yl)cyclohexyl)piperazine-1-carboxylate (1.5 g, 2.43 mmol, 82% yield) was obtained as a yellow solid. MS (ESI) m/z: 618.2 [M+H]⁺. ¹H NMR (400 MHz, CDCl₃) δ=10.70 (br s, 1H), 8.93-8.67 (m, 4H), 7.37 (br s, 1H), 7.08 (br s, 1H), 4.88-4.69 (m, 1H), 4.30 (br s, 2H), 4.26-4.18 (m, 1H), 3.61 (br s, 4H), 2.84-2.53 (m, 4H), 2.28-2.09 (m, 2H), 2.06-1.98 (m, 2H), 1.63 (br s, 4H), 1.54-1.43 (m, 15H).

### Step 7:

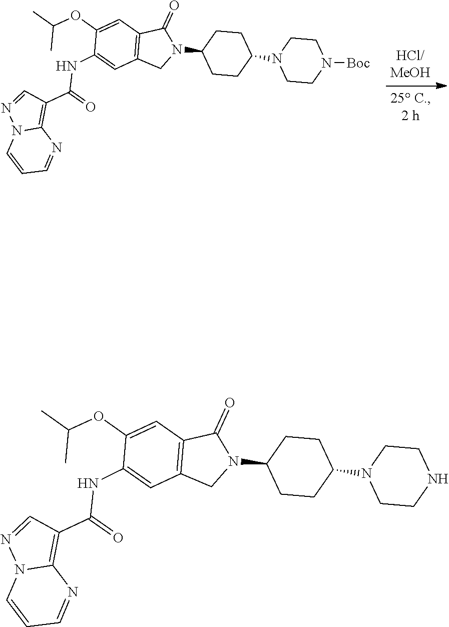 [SMILES: CC(C)Oc1cc2c(cc1NC(=O)c1cnn3cccnc13)CN([C@H]1CC[C@H](N3CCN(C(=O)OC(C)(C)C)CC3)CC1)C2=O ; CC(C)Oc1cc2c(cc1NC(=O)c1cnn3cccnc13)CN([C@H]1CC[C@H](N3CCNCC3)CC1)C2=O]

To a solution of tert-butyl 4-((1r,4r)-4-(6-isopropoxy-1-oxo-5-(pyrazolo[1,5-a]pyrimidine-3-carboxamido)isoindolin-2-yl)cyclohexyl)piperazine-1-carboxylate (1.5 g, 2.43 mmol, 1 eq) in methanol (15 mL) was added a solution of hydrogen chloride in methanol (4 M, 15 mL, 24.71 eq). The mixture was stirred at 25° C. for 2 h. The reaction mixture was concentrated under reduced pressure to give a residue which was suspended in a 10:1 solution of dichloromethane:methanol (500 mL). The pH of the solution was adjusted to 7-8 with ammonium hydroxide, and was then dried over anhydrous sodium sulfate, filtered, and concentrated under reduced pressure to afford N-(6-isopropoxy-1-oxo-2-((1r,4r)-4-(piperazin-1-yl)cyclohexyl)isoindolin-5-yl)pyrazolo[1,5-a]pyrimidine-3-carboxamide (1.2 g, 2.32 mmol, 95% yield) as a yellow solid which was used in the next step without further purification. MS (ESI) m/z: 518.3 [M+H]⁺.

### Step 8:

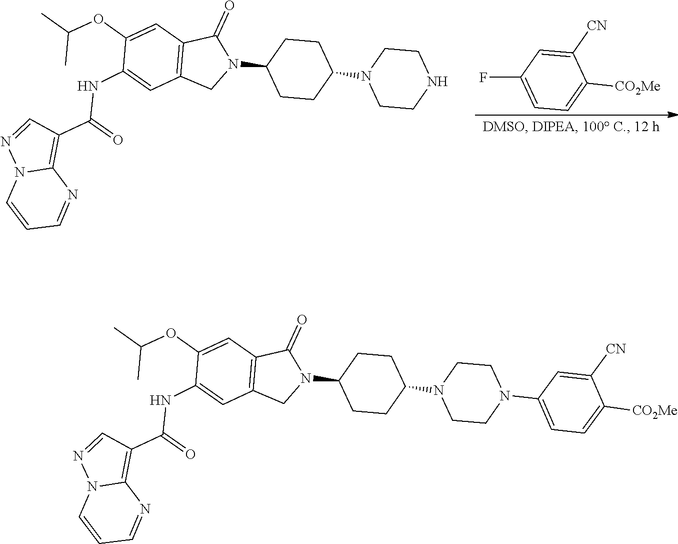 [SMILES: CC(C)Oc1cc2c(cc1NC(=O)c1cnn3cccnc13)CN([C@H]1CC[C@H](N3CCNCC3)CC1)C2=O ; COC(=O)c1ccc(F)cc1C#N ; COC(=O)c1ccc(N2CCN([C@H]3CC[C@H](N4Cc5cc(NC(=O)c6cnn7cccnc67)c(OC(C)C)cc5C4=O)CC3)CC2)cc1C#N]

To a solution of N-(6-isopropoxy-1-oxo-2-((1r,4r)-4-(piperazin-1-yl)cyclohexyl)isoindolin-5-yl)pyrazolo[1,5-a]pyrimidine-3-carboxamide (600 mg, 1.16 mmol, 1 eq) and methyl 2-cyano-4-fluoro-benzoate (420 mg, 2.34 mmol, 2.02 eq) in dimethyl sulfoxide (10 mL) was added diisopropylethylamine (750 mg, 5.80 mmol, 1.01 mL, 5.01 eq). The mixture was stirred at 100° C. for 12 h. The reaction mixture was poured into water (50 mL) and extracted with ethyl acetate (20 mL×3), the combined organic phase was combined and washed with saturated brine (20 mL×3), dried over anhydrous sodium sulfate, filtered, and concentrated under reduced pressure to give a residue. The residue was purified by silica gel column chromatography (dichloromethane:methanol=50/1 to 10/1) to afford methyl 2-cyano-4-(4-((1r,4r)-4-(6-isopropoxy-1-oxo-5-(pyrazolo[1,5-a]pyrimidine-3-carboxamido)isoindolin-2-yl)cyclohexyl)piperazin-1-yl)benzoate (700 mg, 1.03 mmol, 89% yield) as a yellow solid. MS (ESI) m/z: 677.2 [M+H]⁺. ¹H NMR (400 MHz, CDCl₃) δ=10.71 (s, 1H), 8.88-8.82 (m, 2H), 8.79 (s, 1H), 8.73 (dd, J=1.6, 4.0 Hz, 1H), 8.01 (d, J=9.2 Hz, 1H), 7.38 (s, 1H), 7.19 (d, J=2.4 Hz, 1H), 7.09 (dd, J=4.0, 6.8 Hz, 1H), 7.03 (dd, J=2.4, 8.8 Hz, 1H), 4.80 (td, J=6.0, 12.0 Hz, 1H), 4.32 (s, 2H), 4.29-4.21 (m, 1H), 3.95 (s, 3H), 3.72-3.27 (m, 4H), 3.05-2.64 (m, 4H), 2.27-1.96 (m, 4H), 1.67 (s, 4H), 1.52 (d, J=6.0 Hz, 6H), 1.26 (s, 1H).

### Step 9:

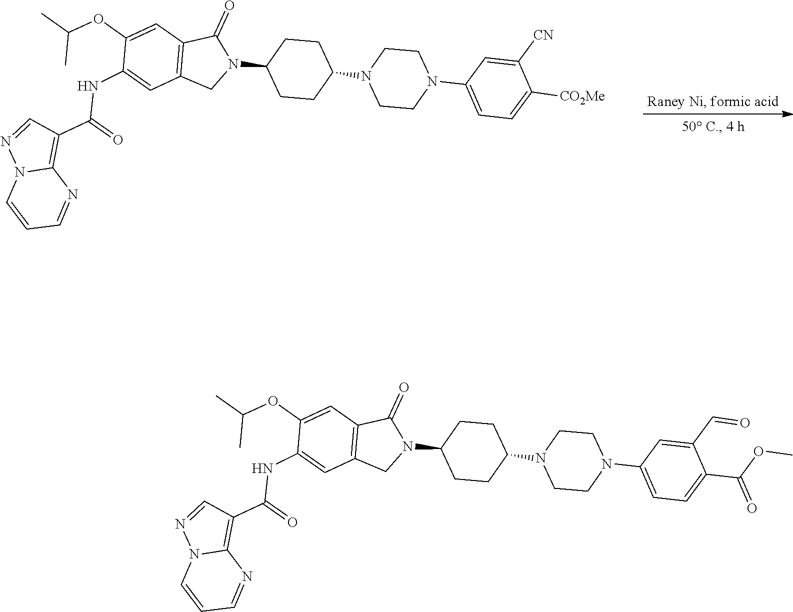 [SMILES: COC(=O)c1ccc(N2CCN([C@H]3CC[C@H](N4Cc5cc(NC(=O)c6cnn7cccnc67)c(OC(C)C)cc5C4=O)CC3)CC2)cc1C#N ; COC(=O)c1ccc(N2CCN([C@H]3CC[C@H](N4Cc5cc(NC(=O)c6cnn7cccnc67)c(OC(C)C)cc5C4=O)CC3)CC2)cc1C=O]

To a solution of methyl 2-cyano-4-(4-((1r,4r)-4-(6-isopropoxy-1-oxo-5-(pyrazolo[1,5-a]pyrimidine-3-carboxamido)isoindolin-2-yl)cyclohexyl)piperazin-1-yl)benzoate (0.5 g, 738.8 mol, 1 eq) in formic acid (15 mL) was added Raney nickel (500.0 mg, 8.52 mmol, 11.53 eq) and the mixture was stirred at 50° C. for 4 h. The reaction was quenched with 10% sodium bicarbonate solution (300 mL) and extracted with ethyl acetate (300 mL×3). The mixture was washed with brine (150 mL), dried over anhydrous sodium sulfate, and concentrated under reduced pressure to give a residue. The residue was purified by preparative thin layer chromatography (silicon dioxide, eluted with dichloromethane/methanol=10/1) to afford methyl 2-formyl-4-(4-((1r,4r)-4-(6-isopropoxy-1-oxo-5-(pyrazolo[1,5-a]pyrimidine-3-carboxamido)isoindolin-2-yl)cyclohexyl)piperazin-1-yl)benzoate (240 mg, 335.4 μmol, 45% yield, 95% purity) as a yellow solid. MS (ESI) m/z: 680.3 [M+H]⁺. ¹H NMR (400 MHz, CDCl₃) δ=10.74 (s, 1H), 10.70 (s, 1H), 8.85 (dd, J=1.6, 6.8 Hz, 1H), 8.83 (s, 1H), 8.78 (s, 1H), 8.72 (dd, J=1.6, 4.0 Hz, 1H), 7.94 (d, J=8.8 Hz, 1H), 7.38 (s, 1H), 7.35 (d, J=2.4 Hz, 1H), 7.08 (dd, J=4.0, 6.8 Hz, 1H), 7.02 (dd, J=2.8, 8.8 Hz, 1H), 4.84-4.76 (m, 1H), 4.31 (s, 2H), 4.23 (br s, 1H), 3.92 (s, 3H), 3.56-3.42 (m, 4H), 2.83 (br s, 4H), 2.62-2.45 (m, 1H), 2.11 (br s, 2H), 2.01 (br s, 2H), 1.66-1.57 (m, 4H), 1.51 (d, J=6.0 Hz, 6H).

### Step 10:

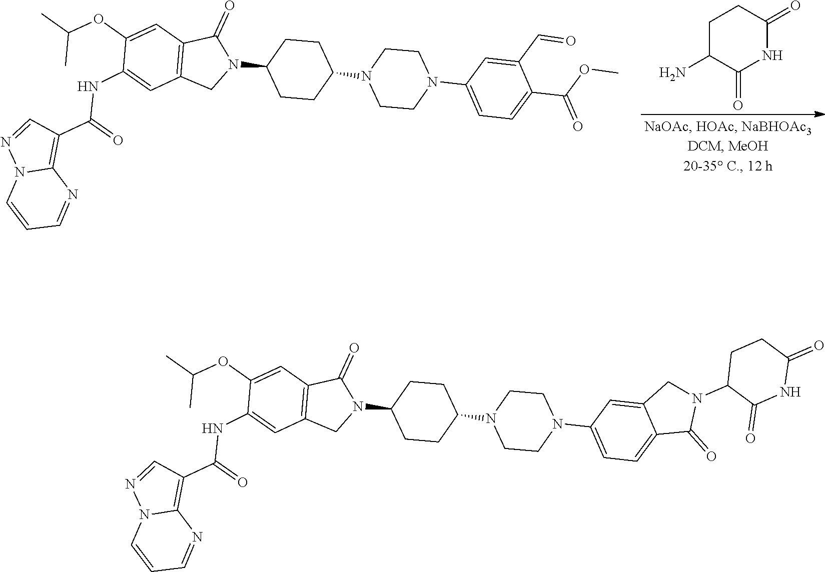 [SMILES: COC(=O)c1ccc(N2CCN([C@H]3CC[C@H](N4Cc5cc(NC(=O)c6cnn7cccnc67)c(OC(C)C)cc5C4=O)CC3)CC2)cc1C=O ; NC1CCC(=O)NC1=O ; CC(C)Oc1cc2c(cc1NC(=O)c1cnn3cccnc13)CN([C@H]1CC[C@H](N3CCN(c4ccc5c(c4)CN(C4CCC(=O)NC4=O)C5=O)CC3)CC1)C2=O]

To a solution of 3-aminopiperidine-2,6-dione (138.0 mg, 838.4 μmol, 2.48 eq, hydrochloride) in dichloromethane (10 mL) and methanol (1 mL) was added sodium acetate (115.0 mg, 1.40 mmol, 4.14 eq). The mixture was stirred at 20° C. for 30 min followed by addition of acetic acid (120.75 mg, 2.01 mmol, 115.0 μL, 5.94 eq) and methyl 2-formyl-4-(4-((1r,4r)-4-(6-isopropoxy-1-oxo-5-(pyrazolo[1,5-a]pyrimidine-3-carboxamido)isoindolin-2-yl)cyclohexyl)piperazin-1-yl)benzoate (230 mg, 338.35 μmol, 1 eq) was added to the mixture. The mixture was stirred at 20° C. for 1.5 h. Then sodium cyanoborohydride (115.0 mg, 1.83 mmol, 5.41 eq) was added to the mixture and the mixture was stirred at 35° C. for 10 h. The reaction mixture was quenched by the addition of water (25 mL) and basified with a saturated aqueous solution of sodium bicarbonate solution (pH=8). The mixture was then extracted with dichloromethane (100 mL×3), and the combined organic phases were washed with brine (100 mL), dried over anhydrous sodium sulfate, and concentrated under reduced pressure to yield a residue. The residue was purified by preparative thin layer chromatography (silicon dioxide, eluted with dichloromethane/methanol=12/1) to afford N-(2-((1r,4r)-4-(4-(2-(2,6-dioxopiperidin-3-yl)-1-oxoisoindolin-5-yl)piperazin-1-yl)cyclohexyl)-6-isopropoxy-1-oxoisoindoin-5-yl)pyrazolo[1,5-a]pyrimidine-3-carboxamide (42.7 mg, 53.4 mol, 1600 yield, 95 purity), obtained as a white solid. MS (ESI) m/z: 760.5 [M+H]⁺. ¹H NMR (400 MHz, CDCl₃) δ=10.71 (s, 1H), 8.88-8.82 (m, 2H), 8.79 (s, 1H), 8.73 (dd, J=1.2, 4.0 Hz, 1H), 8.05 (s, 1H), 7.76 (d, J=8.4 Hz, 1H), 7.39 (s, 1H), 7.09 (dd, J=4.0, 6.8 Hz, 1H), 7.01 (br d, J=7.2 Hz, 1H), 6.91 (s, 1H), 5.21 (dd, J=4.8, 12.8 Hz, 1H), 4.85-4.77 (m, 1H), 4.46-4.40 (m, 1H), 4.35-4.20 (m, 4H), 3.59-3.42 (m, 4H), 3.08-2.75 (m, 6H), 2.43-2.13 (m, 5H), 2.09-2.01 (m, 2H), 1.76-1.68 (m, 4H), 1.52 (d, J=6.0 Hz, 6H).

TABLE 5
The following compounds may be prepared in an analogous fashion as described for the
synthesis of Example 273.

| | | ¹H NMR |
|---|---|---|
| | | All spectra acquired at 400 MHz unless |
| Example | MS | otherwise indicated |
| Example 273 | See Above | |
| 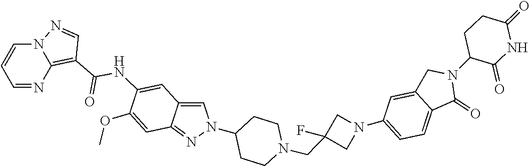 [SMILES: COc1cc2nn(C3CCN(CC4(F)CN(c5ccc6c(c5)CN(C5CCC(=O)NC5=O)C6=O)C4)CC3)cc2cc1NC(=O)c1cnn2cccnc12] | 721.4 | DMSO-d₆ δ = 10.98 (s, 1H), 10.55 (s, 1H), 9.40-9.36 (m, 1H), 8.98-8.89 (m, 1H), 8.72-8.68 (m, 2H), 8.45-8.43 (m, 1H), 8.33-8.31 (m, 1H), 7.58-7.49 (m, 1H), 7.39-7.26 (m, 1H), 7.14-7.09 (m, 1H), 6.67-6.63 (m, 1H), 6.61-6.57 (m, 1H), 5.09-5.00 (m, 1H), 4.47-4.09 (m, 6H), 4.03 (s, 3H), 4.02-3.97 (m, 1H), 3.07 (br d, J = 11.6 Hz, 3H), 3.00-2.89 (m, 4H), 2.24-2.06 (m, 6H), 2.04-1.90 (m, 2H) |
| Example 274 | | |
| 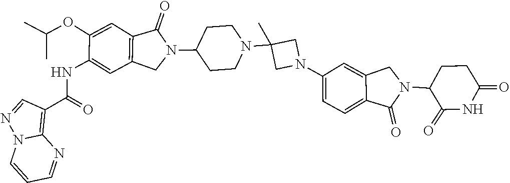 [SMILES: CC(C)Oc1cc2c(cc1NC(=O)c1cnn3cccnc13)CN(C1CCN(C3(C)CN(c4ccc5c(c4)CN(C4CCC(=O)NC4=O)C5=O)C3)CC1)C2=O] | 746.3 | DMSO-d₆ δ = 10.95 (s, 1 H) 10.75 (s, 1 H) 9.40 (dd, J = 6.8, 1.2 Hz, 1 H) 8.89 (dd, J = 4.4, 1.6 Hz, 1 H) 8.73 (s, 1 H) 8.72 (s, 1 H) 7.50 (d, J = 8.4 Hz, 1 H) 7.34-7.37 (m, 1 H) 7.33 (s, 1 H) 6.54 (s, 1 H) 6.51 (d, J = 8.4 Hz, 1 H) 5.04 (dd, J = 13.2, 4.8 Hz, 1 H) 4.89 (m, 1 H) 4.40 (s, 2 H) 4.13-4.35 (m, 2 H) 4.01 (br s, 1 H) 3.63-3.75 (m, 4 H) 2.85-2.97 (m, 1 H) 2.76 (m, 2 H) 2.52- 2.68 (m, 1 H) 2.24-2.42 (m, 3 H) 1.92- 2.01 (m, 1 H) 1.68-1.81 (m, 4 H) 1.43 (d, J = 6.0 Hz, 6 H) 1.35 (s, 3 H) |
| Example 275 | | |
| 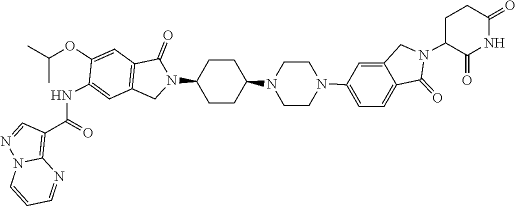 [SMILES: CC(C)Oc1cc2c(cc1NC(=O)c1cnn3cccnc13)CN([C@H]1CC[C@@H](N3CCN(c4ccc5c(c4)CN(C4CCC(=O)NC4=O)C5=O)CC3)CC1)C2=O] | 760.3 | DMSO-d₆ δ = 10.95 (s, 1H), 10.73 (s, 1H), 9.38 (d, J = 7.2 Hz, 1H), 8.88 (d, J = 4.0 Hz, 1H), 8.78-8.64 (m, 2H), 7.54 (d, J = 8.4 Hz, 1H), 7.41-7.26 (m, 2H), 7.17- 6.98 (m, 2H), 5.05 (dd, J = 4.8, 13.2 Hz, 1H), 4.94-4.79 (m, 1H), 4.48-4.32 (m, 3H), 4.27-4.05 (m, 3H), 3.02-2.84 (m, 3H), 2.63-2.54 (m, 6H), 2.27-2.15 (m, 2H), 2.08 (d, J = 11.2 Hz, 2H), 1.98-1.86 (m, 3H), 1.55-1.38 (m, 10H) |
| Example 276 | | |
| 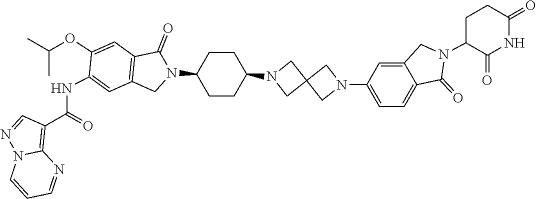 [SMILES: CC(C)Oc1cc2c(cc1NC(=O)c1cnn3cccnc13)CN([C@H]1CC[C@@H](N3CC4(CN(c5ccc6c(c5)CN(C5CCC(=O)NC5=O)C6=O)C4)C3)CC1)C2=O] | 772.3 | DMSO-d₆ δ = 10.94 (s, 1 H), 10.74 (s, 1 H), 9.40 (d, J = 7.2 Hz, 1 H), 8.86-8.92 (m, 1 H), 8.75 (d, J = 3.6 Hz, 2 H), 8.16 (s, 1 H), 7.50 (d, J = 8.4 Hz, 1 H), 7.36 (dd, J = 7.2, 4.0 Hz, 1 H), 7.32 (s, 1 H), 7.25 (s, 1 H), 7.18-7.22 (m, 1 H), 6.47- 6.55 (m, 2 H), 5.04 (dd, J = 13.2, 5.2 Hz, 1 H), 4.89 (dt, J = 12.0, 6.0 Hz, 1 H), 4.41 (s, 2 H), 4.28-4.34 (m, 1 H), 4.15-4.21 (m, 1 H), 4.01 (s, 6 H), 2.86-2.94 (m, 1 H), 2.58 (d, J = 17.2 Hz, 1 H), 2.28-2.36 (m, 2 H), 1.91-2.04 (m, 2 H), 1.79-1.90 (m, 2 H), 1.70 (d, J = 13.6 Hz, 2 H), 1.45-1.52 |
| Example 277 | | (m, 2 H), 1.44 (d, J = 6.0 Hz, 6 H), 1.32- |
| | | 1.42 (m, 2H), 1.23 (s, 2 H). |
| 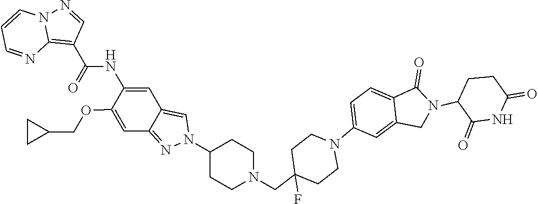 [SMILES: O=C1CCC(N2Cc3cc(N4CCC(F)(CN5CCC(n6cc7cc(NC(=O)c8cnn9cccnc89)c(OCC8CC8)cc7n6)CC5)CC4)ccc3C2=O)C(=O)N1] | 789.2 | DMSO-d₆ δ = 10.95 (s, 1H), 10.66 (s, 1H), 9.39 (d, J = 6.8 Hz, 1H), 8.88 (d, J = 4.0 Hz, 1H), 8.74 (d, J = 10.0 Hz, 1H), 8.30 (s, 1H), 7.54 (d, J = 8.4 Hz, 1H), 7.34 (s, 1H), 7.12-7.10 (m, 2H), 7.03 (s, 1H), 5.08-5.05 (m, 1H), 4.38-4.32 (m, 3H), 3.73 (d, J = 5.6Hz, 1H), 3.34-3.31 (m, 2H), 2.99-2.96 (m, 2H), 2.95-2.85 (m, 1H), 2.64-2.53 (m, 4H), 2.34-2.30 (m, 3H), 2.06-1.95 (m, 8H), 1.80-1.70 (m, 2H), 1.50 (s, 1H), 0.73 (d, J = 8.8 Hz, 2H), 0.48 (d, J = 8.8 Hz, 2H). |
| Example 278 | | |
| 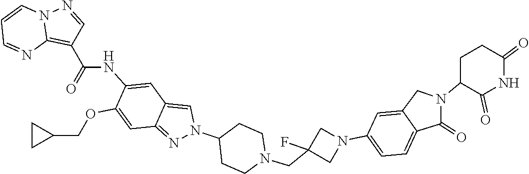 [SMILES: O=C1CCC(N2Cc3cc(N4CC(F)(CN5CCC(n6cc7cc(NC(=O)c8cnn9cccnc89)c(OCC8CC8)cc7n6)CC5)C4)ccc3C2=O)C(=O)N1] | 761.3 | DMSO-d₆ δ = 10.94 (s, 1 H) 10.65 (s, 1 H) 9.38 (dd, J = 6.8, 1.6 Hz, 1 H) 8.87 (dd, J = 4.0, 1.6 Hz, 1 H) 8.74 (s, 1 H) 8.71 (s, 1 H) 8.30 (s, 1 H) 7.53 (d, J = 8.0 Hz, 1 H) 7.32 (dd, J = 6.8, 4.0 Hz, 1 H) 7.04 (s, 1 H) 6.64 (s, 1 H) 6.57-6.61 (m, 1 H) 5.02- 5.08 (m, 1H) 4.29-4.42 (m, 2 H) 4.09- 4.23 (m, 3H) 3.99-4.04 (m, 3 H) 3.02- 3.12 (m, 2H) 2.83-3.00 (m, 3 H) 2.55- 2.63 (m, 1 H) 2.39 (br dd, J = 9.2, 3.2 Hz, 2 H) 2.06-2.14 (m, 3 H) 1.89-2.03 (m, 2 H) 1.45-1.52 (m, 1 H) 1.23 (s, 2 H) 0.65- |
| Example 279 | | 0.73 (m, 2 H) 0.41-0.51 (m, 2 H). |
| 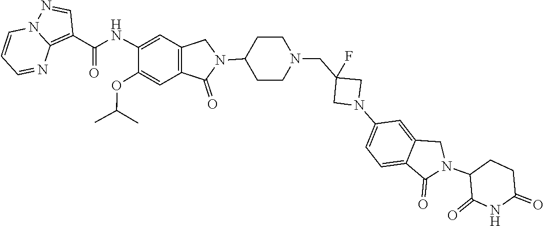 [SMILES: CC(C)Oc1cc2c(cc1NC(=O)c1cnn3cccnc13)CN(C1CCN(CC3(F)CN(c4ccc5c(c4)CN(C4CCC(=O)NC4=O)C5=O)C3)CC1)C2=O] | 764.5 | CDCl3 δ = 10.71 (s, 1H), 8.89-8.83 (m, 2H), 8.81 (s, 1H), 8.75-8.71 (m, 1H), 8.22 (br s, 1H), 7.73 (s, 1H), 7.38 (s, 1H), 7.09 (dd, J = 4.4, 7.2 Hz, 1H), 6.54 (dd, J = 1.6, 8.4 Hz, 1H), 6.47 (s, 1H), 5.23 (dd, J = 5.6, 13.2 Hz, 1H), 4.80 (s, 1H), 4.47-4.39 (m, 1H), 4.33-4.22 (m, 4H), 4.15-4.01 (m, 4H), 3.07 (br d, J = 11.6 Hz, 2H), 2.97-2.79 (m, 4H), 2.35 (br s, 2H), 2.38-2.37 (m, 1H), 2.38-2.29 (m, 1H), 2.27-2.17 (m, 1H), 1.82 (br d, J = 5.6 Hz, 4H), 1.52 (d, J = 6.0 Hz, 6H). |
| Example 280 | | |
| 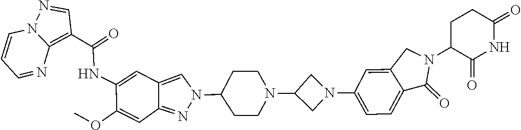 [SMILES: COc1cc2nn(C3CCN(C4CN(c5ccc6c(c5)CN(C5CCC(=O)NC5=O)C6=O)C4)CC3)cc2cc1NC(=O)c1cnn2cccnc12] | 689.3 | DMSO-d₆ δ = 10.96 (s, 1 H), 10.55 (s, 1 H), 9.38 (d, J = 6.8 Hz, 1 H), 8.96 (d, J = 2.8 Hz, 1 H), 8.69 (d, J = 9.6 Hz, 2 H), 8.33 (s, 1 H), 7.51 (d, J = 8.0 Hz, 1 H), 7.28-7.40 (m, 1 H), 7.11 (s, 1 H), 6.44- 6.59 (m, 2 H), 4.99-5.09 (m, 1 H), 4.43 (s, 1 H), 4.16-4.34 (m, 2 H), 4.00-4.08 (m, 5 H), 3.74 (s, 2 H), 2.83-3.02 (m, 4 H), 2.58- 2.74 (m, 1 H), 2.31-2.38 (m, 1 H), 2.07- |
| Example 281 | | 2.14 (m, 6 H), 1.95 (s, 1 H). |
| 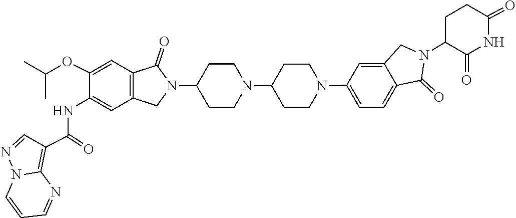 [SMILES: CC(C)Oc1cc2c(cc1NC(=O)c1cnn3cccnc13)CN(C1CCN(C3CCN(c4ccc5c(c4)CN(C4CCC(=O)NC4=O)C5=O)CC3)CC1)C2=O] | 760.3 | DMSO-d₆ δ = 10.94 (s, 1 H), 10.75 (s, 1 H), 9.39 (dd, J = 7.2, 1.6 Hz, 1 H), 8.89 (dd, J = 4.0, 1.6 Hz, 1 H), 8.73 (s, 2 H), 7.50 (d, J = 8.4 Hz, 1 H), 7.28-7.39 (m, 2 H), 6.99-7.10 (m, 2 H), 5.04 (dd, J = 13.2, 5.2 Hz, 1 H), 4.89 (q, J = 6.0 Hz, 1 H), 4.41 (s, 2 H), 4.17-4.36 (m, 2 H), 3.87- 4.03 (m, 3 H), 2.79-3.02 (m, 5 H), 2.53- 2.62 (m, 2 H), 2.20-2.42 (m, 3 H), 1.92- 2.01 (m, 1 H), 1.68-1.87 (m, 6 H), 1.48- 1.60 (m, 2 H), 1.44 (d, J = 6.0 Hz, 6 H). |
| Example 282 | | |
| 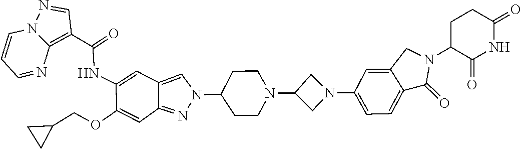 [SMILES: O=C1CCC(N2Cc3cc(N4CC(N5CCC(n6cc7cc(NC(=O)c8cnn9cccnc89)c(OCC8CC8)cc7n6)CC5)C4)ccc3C2=O)C(=O)N1] | 729.3 | DMSO-d₆ δ = 10.98 (s, 1H), 10.63 (d, J = 3.6 Hz, 1H), 9.40-9.36 (m, 1H), 8.87 (dd, J = 4.0, 1.6, 1H), 8.74 (s, 1H), 8.72 (s, 1H), 8.35-8.28 (m, 1H), 7.49 (br d, J = 1.6 Hz, 1H), 7.29 (s, 1H), 7.06-7.02 (m, 1H), 6.57-6.54 (m, 1H), 6.53-6.49 (m, 1H), 4.99 (br d, J = 6.4 Hz, 1H), 4.47- 4.38 (m, 1H), 4.36-4.16 (m, 2H), 4.09- 3.98 (m, 4H), 3.79-3.70 (m, 2H), 3.03- 2.93 (m, 2H), 2.84 (br d, J = 4.8 Hz, 1H), |
| Example 283 | | 2.20-2.19 (m, 1H), 2.20-2.07 (m, 6H), |
| | | 2.07-1.91 (m, 2H), 1.56-1.43 (m, 1H), |
| | | 0.76-0.63 (m, 2H), 0.51-0.36 (m, 2H) |
| 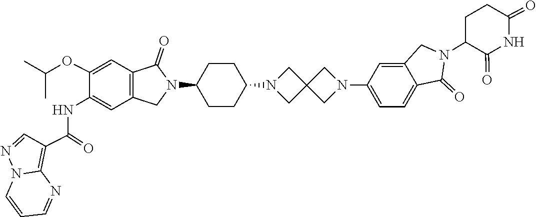 [SMILES: CC(C)Oc1cc2c(cc1NC(=O)c1cnn3cccnc13)CN([C@H]1CC[C@H](N3CC4(CN(c5ccc6c(c5)CN(C5CCC(=O)NC5=O)C6=O)C4)C3)CC1)C2=O] | 722.4 | DMSO-d₆ δ = 10.93 (s, 1 H) 10.75 (s, 1 H) 9.40 (d, J = 7.2 Hz, 1 H) 8.89 (d, J = 3.6 Hz, 1 H) 8.74 (s, 2 H) 7.49 (d, J = 8.0 Hz, 1 H) 7.32-7.38 (m, 2 H) 6.52 (s, 1 H) 6.47 (br d, J = 8.8 Hz, 1 H) 5.03 (dd, J = 13.6, 4.8 Hz, 1 H) 4.86-4.94 (m, 1 H) 4.39 (s, 2 H) 4.15-4.33 (m, 2 H) 4.09 (m, 2 H) 3.97 (br s, 5 H) 3.17 (d, J = 5.2 Hz, 4 H) 2.83- 2.96 (m, 1 H) 2.60 (br s, 2 H) 1.93-2.01 (m, 2 H) 1.71-1.84 (m, 4 H) 1.58 (br d, J = 12.8 Hz, 2H) 1.44 (d, J = 6.0 Hz, 6H). |
| Example 284 | | |

### Preparation of Example 285: N-(6-(cyclopropylmethoxy)-2-(1-((1-(2-(2,6-dioxopiperidin-3-yl)-4-methoxy-1,3-dioxoisoindolin-5-yl)azetidin-3-yl)methyl)piperidin-4-yl)-2H-indazol-5-yl)pyrazolo[1,5-a]pyrimidine-3-carboxamide

### Step 1:

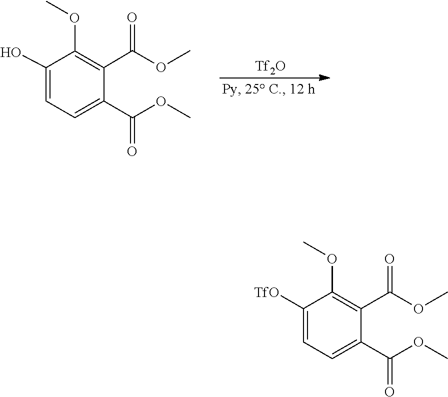 [SMILES: "Tf2O" ; COC(=O)c1ccc(O)c(OC)c1C(=O)OC ; COC(=O)c1ccc(OS(=O)(=O)C(F)(F)F)c(OC)c1C(=O)OC]

To a solution of dimethyl 4-hydroxy-3-methoxy-benzene-1,2-dicarboxylate (1 g, 4.16 mmol, 1 eq) in pyridine (20 mL) was added trifluoromethanesulfonic anhydride (1.41 g, 5.00 mmol, 0.82 mL, 1.2 eq) at 0° C. The mixture was stirred at 25° C. for 12 h. To the reaction mixture was added water (10 mL) and the mixture was extracted with ethyl acetate (20 mL×3). The combined organic phase was washed with brine (20 mL), dried over anhydrous sodium sulfate, filtered, and concentrated in vacuo. The residue was purified by column chromatography (silica gel, eluted with petroleum ether/ethyl acetate=20/1 to 5/1) to afford dimethyl 3-methoxy-4-(((trifluoromethyl)sulfonyl)oxy)phthalate (1.5 g, 97% yield) as a yellow oil. MS (ESI) m/z: 341.0 [M-OMe]⁺.

### Step 2:

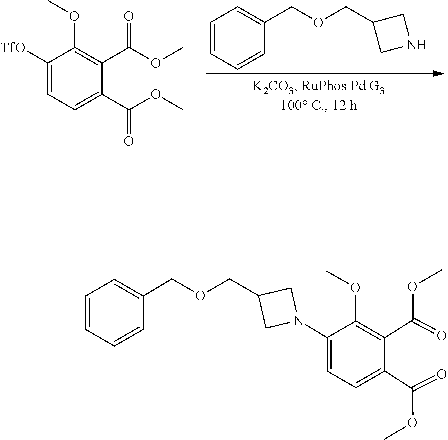

A mixture of dimethyl 3-methoxy-4-(((trifluoromethyl)sulfonyl)oxy)phthalate (1 g, 2.69 mmol, 1 eq), 3-(benzyloxymethyl)azetidine (571 mg, 3.22 mmol, 1.2 eq), RuPhos Pd G3 (112 mg, 0.13 mmol, 0.05 eq), potassium carbonate (742 mg, 5.37 mmol, 2 eq) in dioxane (20 mL) was degassed and purged with nitrogen in 3 cycles. The mixture was stirred at 100° C. for 12 h under an atmosphere of nitrogen. The reaction mixture was concentrated in vacuo to afford a residue which was purified by column chromatography (silica gel, eluted with petroleum ether/ethyl acetate=10/1 to 1/1). Dimethyl 4-(3-((benzyloxy)methyl)azetidin-1-yl)-3-methoxyphthalate (0.8 g, 75% yield) was obtained as a brown oil. MS (ESI) m/z: 400.3 [M+H]⁺.

### Step 3:

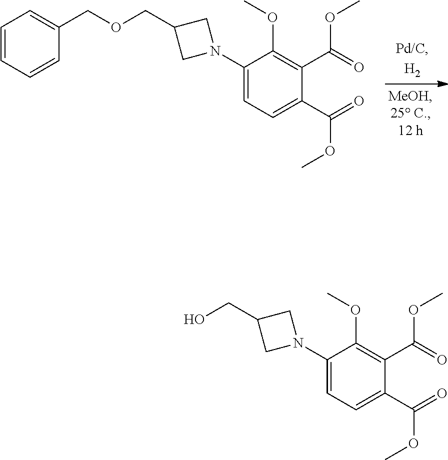 [SMILES: COC(=O)c1ccc(N2CC(COCc3ccccc3)C2)c(OC)c1C(=O)OC ; COC(=O)c1ccc(N2CC(CO)C2)c(OC)c1C(=O)OC]

To a solution of dimethyl 4-(3-((benzyloxy)methyl)azetidin-1-yl)-3-methoxyphthalate (800 mg, 2.00 mmol, 1 eq) in methanol (10 mL) was added palladium/carbon (200 mg, 10% purity) under an atmosphere of nitrogen. The suspension was degassed under vacuum and purged with hydrogen over several cycles. The mixture was stirred under hydrogen (15 psi) at 25° C. for 12 h. The reaction mixture was concentrated in vacuo to give a residue. The residue was purified by column chromatography (silica gel, eluted with petroleum ether/ethyl acetate=10/1 to 1/1) to afford dimethyl 4-(3-(hydroxymethyl)azetidin-1-yl)-3-methoxyphthalate (600 mg, 97% yield) as a white solid. MS (ESI) m/z: 310.0 [M+H]⁺.

### Step 4:

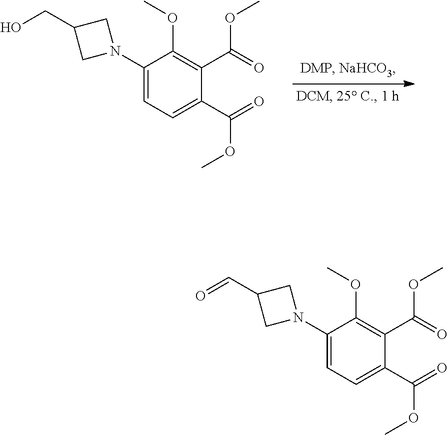 [SMILES: COC(=O)c1ccc(N2CC(CO)C2)c(OC)c1C(=O)OC ; COC(=O)c1ccc(N2CC(C=O)C2)c(OC)c1C(=O)OC]

To a solution of dimethyl 4-(3-(hydroxymethyl)azetidin-1-yl)-3-methoxyphthalate (400 mg, 1.29 mmol, 1 eq) in dichloromethane (10 mL) was added Dess-Martin periodinane (822 mg, 1.94 mmol, 1.5 eq) and sodium bicarbonate (1.09 g, 12.9 mmol, 10 eq). The mixture was stirred at 25° C. for 1 h. To the reaction mixture was added water (10 mL) and the mixture was extracted with ethyl acetate (20 mL×3). The combined organic phases were washed with brine (20 mL), dried over anhydrous sodium sulfate, filtered, and concentrated in vacuo. The residue was purified by column chromatography (silica gel, eluted with petroleum ether/ethyl acetate=5/1 to 1/1) to afford dimethyl 4-(3-formylazetidin-1-yl)-3-methoxyphthalate (300 mg, 75% yield) as a white solid. MS (ESI) m/z: 308.1 [M+H]⁺.

### Step 5:

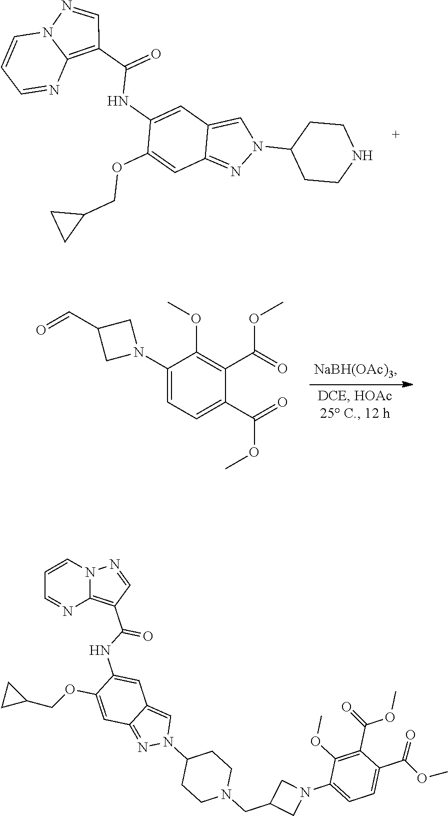 [SMILES: O=C(Nc1cc2cn(C3CCNCC3)nc2cc1OCC1CC1)c1cnn2cccnc12 ; COC(=O)c1ccc(N2CC(C=O)C2)c(OC)c1C(=O)OC ; COC(=O)c1ccc(N2CC(CN3CCC(n4cc5cc(NC(=O)c6cnn7cccnc67)c(OCC6CC6)cc5n4)CC3)C2)c(OC)c1C(=O)OC]

To a solution of dimethyl 4-(3-formylazetidin-1-yl)-3-methoxyphthalate (214 mg, 0.69 mmol, 1 eq), N-(6-(cyclopropylmethoxy)-2-(piperidin-4-yl)-2H-indazol-5-yl)pyrazolo[1,5-a]pyrimidine-3-carboxamide (300 mg, 0.69 mmol, 1 eq) in dichloroethane (15 mL) was added acetic acid (42 mg, 0.69 mmol, 0.046 mL, 1 eq). The reaction mixture was stirred at 25° C. for 1 h, and then sodium triacetoxyborohydride (442 mg, 2.09 mmol, 3 eq) was added. The reaction was stirred at 25° C. for 11 h. To the reaction mixture was added water (10 mL) and the mixture was extracted with ethyl acetate (20 mL×3). The combined organic phase was washed with brine (20 mL), dried over anhydrous sodium sulfate, filtered, and concentrated in vacuo. The residue was purified by column chromatography (silica gel, eluted with dichloromethane/methanol=30/1 to 10:1) to afford dimethyl 4-(3-((4-(6-(cyclopropylmethoxy)-5-(pyrazolo[1,5-a]pyrimidine-3-carboxamido)-2H-indazol-2-yl)piperidin-1-yl)methyl)azetidin-1-yl)-3-methoxyphthalate (310 mg, 62% yield) as a white solid. MS (ESI) m/z: 723.1 [M+H]⁺.

### Step 6:

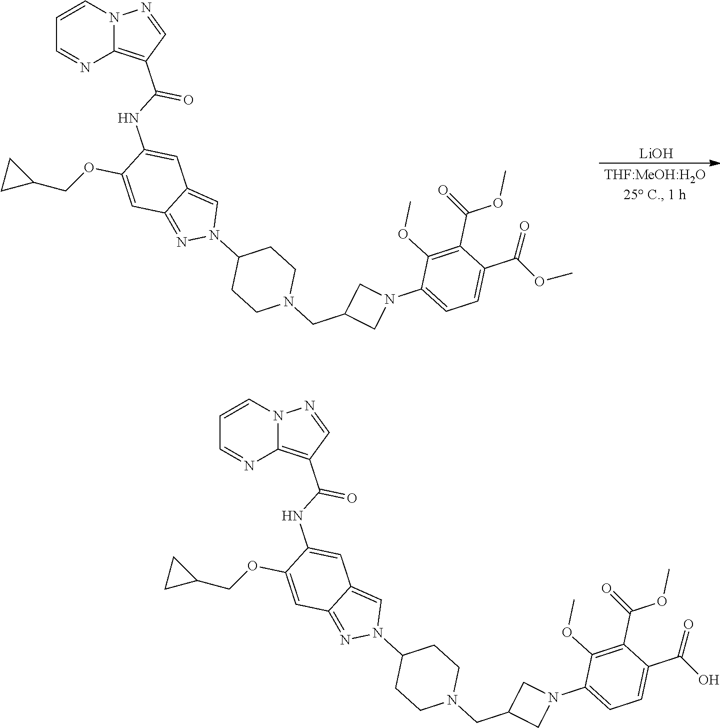 [SMILES: COC(=O)c1ccc(N2CC(CN3CCC(n4cc5cc(NC(=O)c6cnn7cccnc67)c(OCC6CC6)cc5n4)CC3)C2)c(OC)c1C(=O)OC ; "LiOH" ; COC(=O)c1c(C(=O)O)ccc(N2CC(CN3CCC(n4cc5cc(NC(=O)c6cnn7cccnc67)c(OCC6CC6)cc5n4)CC3)C2)c1OC]

To a solution of dimethyl 4-(3-((4-(6-(cyclopropylmethoxy)-5-(pyrazolo[1,5-a]pyrimidine-3-carboxamido)-2H-indazol-2-yl)piperidin-1-yl)methyl)azetidin-1-yl)-3-methoxyphthalate (310 mg, 0.43 mmol, 1 eq) in water (2 mL), tetrahydrofuran (2 mL) and methanol (2 mL) was added lithium hydroxide monohydrate (90 mg, 2.14 mmol, 5 eq). The mixture was stirred at 25° C. for 1 h. The reaction mixture was concentrated in vacuo to afford 4-(3-((4-(6-(cyclopropylmethoxy)-5-(pyrazolo[1,5-a]pyrimidine-3-carboxamido)-2H-indazol-2-yl)piperidin-1-yl)methyl)azetidin-1-yl)-3-methoxy-2-(methoxycarbonyl)benzoic acid (300 mg, crude) as a white solid. MS (ESI) m/z: 709.1 [M+H]⁺.

### Step 7:

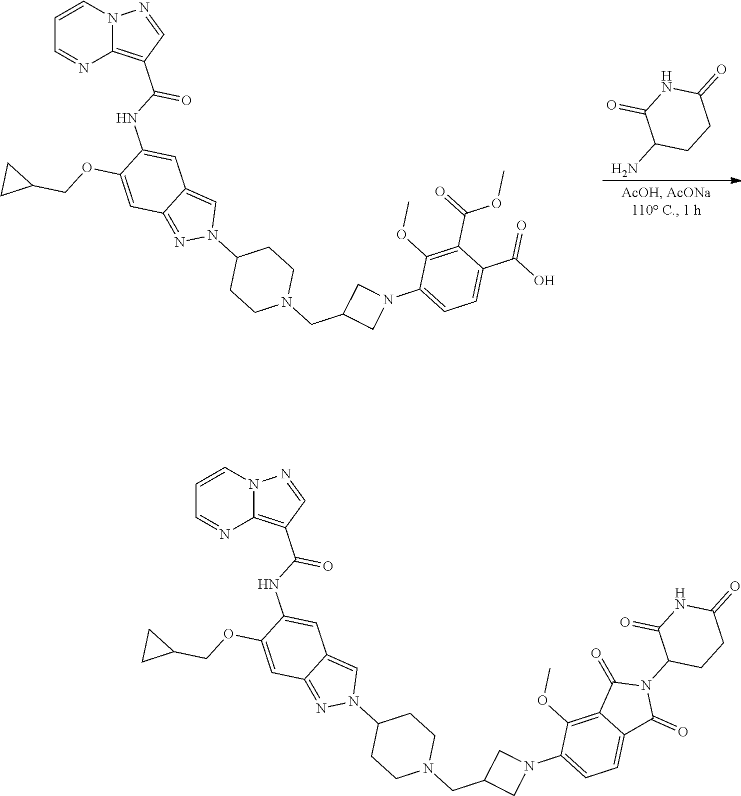 [SMILES: COC(=O)c1c(C(=O)O)ccc(N2CC(CN3CCC(n4cc5cc(NC(=O)c6cnn7cccnc67)c(OCC6CC6)cc5n4)CC3)C2)c1OC ; NC1CCC(=O)NC1=O ; COc1c(N2CC(CN3CCC(n4cc5cc(NC(=O)c6cnn7cccnc67)c(OCC6CC6)cc5n4)CC3)C2)ccc2c1C(=O)N(C1CCC(=O)NC1=O)C2=O]

To a solution of 4-(3-((4-(6-(cyclopropylmethoxy)-5-(pyrazolo[1,5-a]pyrimidine-3-carboxamido)-2H-indazol-2-yl)piperidin-1-yl)methyl)azetidin-1-yl)-3-methoxy-2-(methoxycarbonyl)benzoic acid (250 mg, 0.35 mmol, 1 eq) in acetic acid (10 mL) was added 3-aminopiperidine-2,6-dione;hydrochloride (116 mg, 0.71 mmol, 2 eq) and sodium acetate (116 mg, 1.41 mmol, 4 eq). The mixture was stirred at 110° C. for 1 h. To the reaction mixture was added water (10 mL) and the mixture was extracted with ethyl acetate (20 mL×3). The combined organic phase was washed with brine (20 mL), dried over anhydrous sodium sulfate, filtered, and concentrated in vacuo. The crude product was purified by preparative HPLC (column: Phenomenex Luna C18 75×30 mm×3 um; mobile phase: [water(0.225% formic acid)-acetonitrile]; B %: 16%-36%, 7 min) to yield N-(6-(cyclopropylmethoxy)-2-(1-((1-(2-(2,6-dioxopiperidin-3-yl)-4-methoxy-1,3-dioxoisoindolin-5-yl)azetidin-3-yl)methyl)piperidin-4-yl)-2H-indazol-5-yl)pyrazolo[1,5-a]pyrimidine-3-carboxamide (17.4 mg, 6% yield, 94% purity) as a yellow solid. MS (ESI) m/z: 787.4 [M+H]⁺. ¹H NMR (400 MHz, DMSO-d₆) δ: 11.07 (s, 1H), 10.65 (s, 1H), 9.37 (dd, J=7.2, 1.6 Hz, 1H), 8.87 (dd, J=4.0, 1.6 Hz, 1H), 8.73 (d, J=11.8 Hz, 2H), 8.31 (s, 1H), 8.28 (s, 1H), 7.42 (d, J=8.0 Hz, 1H), 7.32 (dd, J=6.8, 4.0 Hz, 1H), 7.04 (s, 1H), 6.62 (d, J=8.0 Hz, 1H), 5.04 (dd, J=12.8, 5.2 Hz, 1H), 4.37 (t, J=8.0 Hz, 1H), 4.24 (t, J=8.0 Hz, 2H), 4.02 (d, J=6.8 Hz, 2H), 3.92 (s, 3H), 3.76-3.84 (m, 2H), 2.98 (d, J=11.2 Hz, 3H), 2.67 (dd, J=3.6, 2.0 Hz, 2H), 2.54-2.61 (m, 2H), 1.97-2.21 (m, 8H), 1.50 (s, 1H), 0.66-0.75 (m, 2H), 0.42-0.50 (m, 2H).

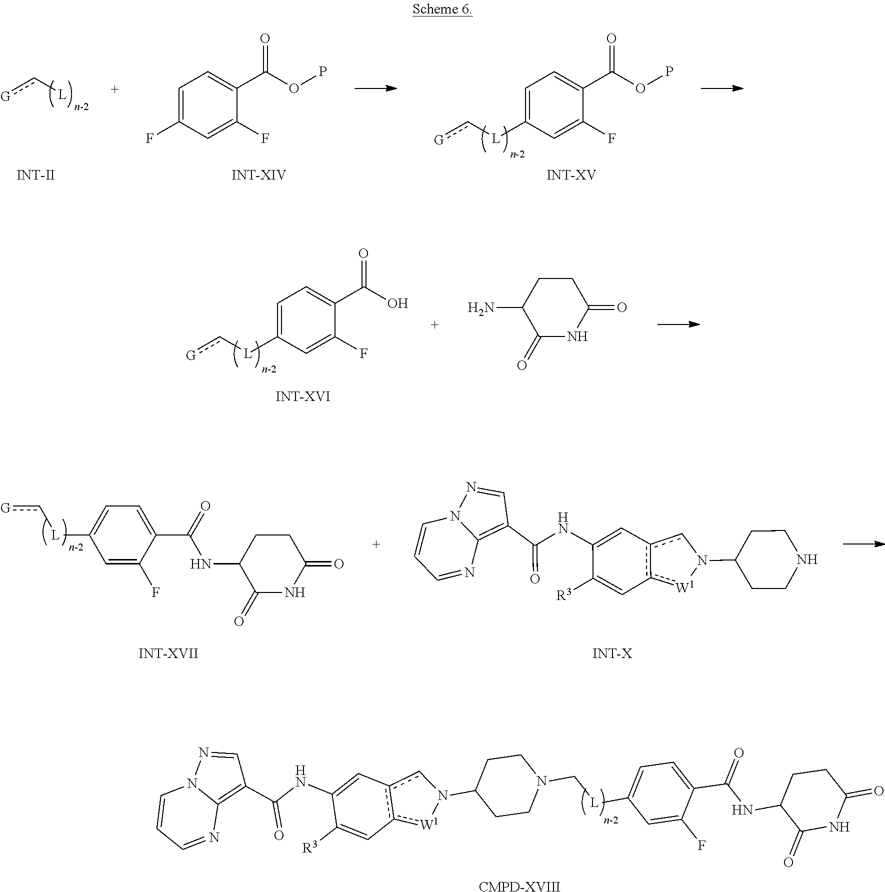

A compound of formula INT-XIV, wherein P is an alkyl ester, may be reacted with a reagent INT-II (commercially available or readily prepared using standard reaction techniques known to one skilled in the art) under nucleophilic aromatic substitution reaction conditions to ultimately provide a compound of formula INT-XVII after saponification and amide formation. A compound of formula INT-XVII can then react with a compound of formula INT-X, wherein R³ is an ether, amine, or cycloalkyl group and W¹ is optionally N or C═O, under reductive amination or alkylation conditions to produce a compound of formula CMPD-XVIII, wherein L and n are as defined herein. Compounds of formula CMPD-XVIII can be furnished from N-alkylation where G is an appropriate leaving group (e.g., OMs, OTs, Cl, etc.) or through reductive amination where G is an aldehyde. When G is a leaving group, suitable reaction conditions are those for an alkylation reaction, e.g., diisopropylethylamine, potassium iodide, DMSO or acetonitrile, 80° C. When G is an aldehyde, suitable reaction conditions are those for a reductive amination reaction, e.g., sodium cyanoborohydride, methanol, dichloromethane, acetic acid, room temperature.

### Preparation of Example 286: N-[2-[1-[[1-[4-[(2,6-dioxo-3-piperidyl)carbamoyl]-3-fluoro-phenyl]-4-piperidyl]methyl]-4-piperidyl]-6-isopropoxy-1-oxo-isoindolin-5-yl]pyrazolo[1,5-a]pyrimidine-3-carboxamide

### Step 1:

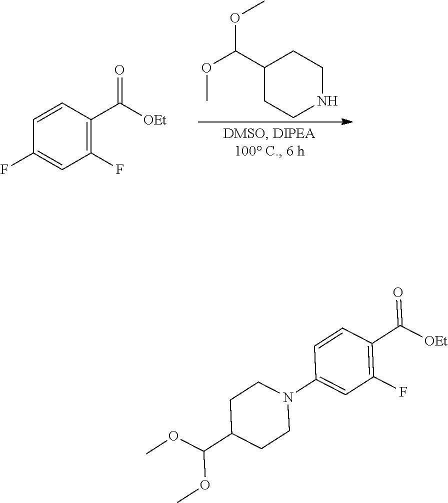 [SMILES: CCOC(=O)c1ccc(F)cc1F ; COC(OC)C1CCNCC1 ; CCOC(=O)c1ccc(N2CCC(C(OC)OC)CC2)cc1F]

To a mixture of ethyl 2,4-difluorobenzoate (0.70 g, 3.76 mmol, 1 eq) and 4-(dimethoxymethyl)piperidine (718 mg, 4.51 mmol, 1.2 eq) in dimethyl sulfoxide (5 mL) was added diisopropylethylamine (1.46 g, 11.28 mmol, 3 eq). The mixture was stirred at 100° C. for 6 h after which the reaction was diluted with water (10 mL) and extracted with ethyl acetate (15 mL×3). The combined organic layers were washed with water (40 mL×2), dried over anhydrous sodium sulfate, filtered, and concentrated under reduced pressure to give a residue. The residue was purified by silica gel column chromatography (eluted with petroleum ether:ethyl acetate=20:1 to 4:1) to afford ethyl 4-[4-(dimethoxymethyl)-1-piperidyl]-2-fluoro-benzoate (0.20 g, 0.61 mmol, 16% yield) as a yellow solid. ¹H NMR (400 MHz, CDCl₃) δ: 7.81 (t, J=8.8 Hz, 1H), 6.63 (dd, J=2.4, 9.2 Hz, 1H), 6.51 (dd, J=2.4, 14.8 Hz, 1H), 4.35 (q, J=7.2 Hz, 2H), 4.07 (d, J=6.8 Hz, 1H), 3.92-3.82 (m, 2H), 3.39 (s, 6H), 2.91-2.80 (m, 2H), 1.91-1.80 (m, 3H), 1.46-1.34 (m, 5H).

### Step 2:

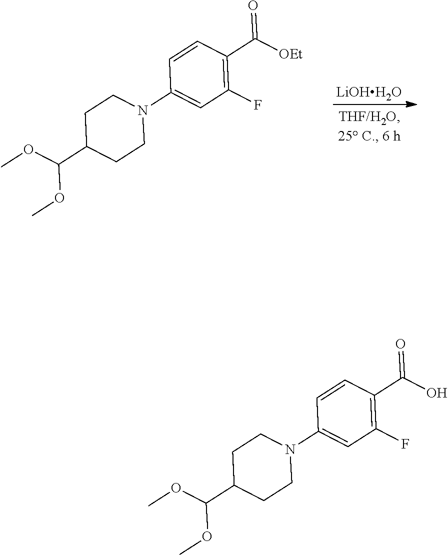 [SMILES: CCOC(=O)c1ccc(N2CCC(C(OC)OC)CC2)cc1F ; "LiOH·H2O" ; COC(OC)C1CCN(c2ccc(C(=O)O)c(F)c2)CC1]

To a mixture of ethyl 4-[4-(dimethoxymethyl)-1-piperidyl]-2-fluoro-benzoate (0.20 g, 0.61 mmol, 1 eq) in tetrahydrofuran (3 mL) and water (3 mL) was added lithium hydroxide hydrate (128 mg, 3.07 mmol, 5 eq). The mixture was stirred at 25° C. for 6 h. The mixture was concentrated under reduced pressure to remove tetrahydrofuran, then the pH was adjusted to 3-4 with diluted hydrochloric acid (1 N). The mixture was filtered, and the cake was concentrated under reduced pressure to give 4-[4-(dimethoxymethyl)-1-piperidyl]-2-fluoro-benzoic acid (0.17 g, 0.57 mmol, 93% yield) as a white solid. MS (ESI) m/z: 298.1 [M+H]⁺.

### Step 3:

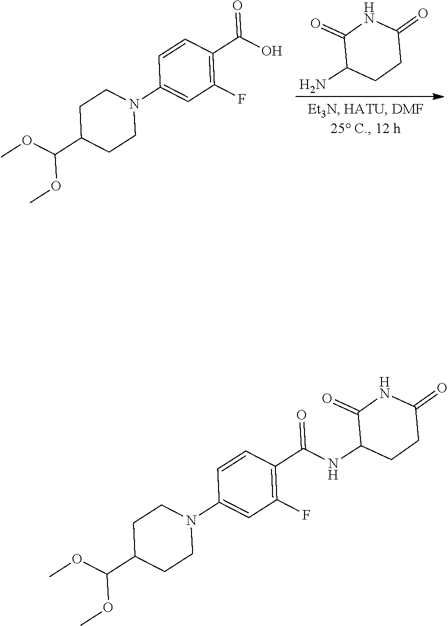 [SMILES: COC(OC)C1CCN(c2ccc(C(=O)O)c(F)c2)CC1 ; NC1CCC(=O)NC1=O ; COC(OC)C1CCN(c2ccc(C(=O)NC3CCC(=O)NC3=O)c(F)c2)CC1]

To a mixture of 4-[4-(dimethoxymethyl)-1-piperidyl]-2-fluorobenzoic acid (0.12 g, 0.40 mmol, 1 eq) and 3-aminopiperidine-2,6-dione;hydrochloride (79 mg, 0.48 mmol, 1.2 eq) in N,N-dimethylformamide (3 mL) was added HATU (230 mg, 0.60 mmol, 1.5 eq) and diisopropylethylamine (156 mg, 1.21 mmol, 3 eq). The mixture was stirred at 25° C. for 12 h. The mixture was diluted with water (5 mL), then extracted with ethyl acetate (10 mL×3). The combined organic layers were washed with water (20 mL×2), dried over anhydrous sodium sulfate, filtered, and concentrated under reduced pressure to give a residue. The residue was purified by preparative TLC (petroleum ether:ethyl acetate=0:1) to give 4-[4-(dimethoxymethyl)-1-piperidyl]-N-(2,6-dioxo-3-piperidyl)-2-fluoro-benzamide (0.13 g, 0.32 mmol, 79% yield) as a gray solid. ¹H NMR (400 MHz, CDCl₃) δ: 8.20 (br s, 1H), 7.96-7.87 (m, 1H), 7.43-7.33 (m, 1H), 6.72-6.63 (m, 1H), 6.55-6.43 (m, 1H), 4.83-4.70 (m, 1H), 4.08-4.00 (m, 1H), 3.89-3.74 (m, 2H), 3.34 (s, 6H), 2.88-2.73 (m, 4H), 2.72-2.61 (m, 1H), 1.98-1.77 (m, 4H), 1.43-1.30 (m, 2H).

### Step 4:

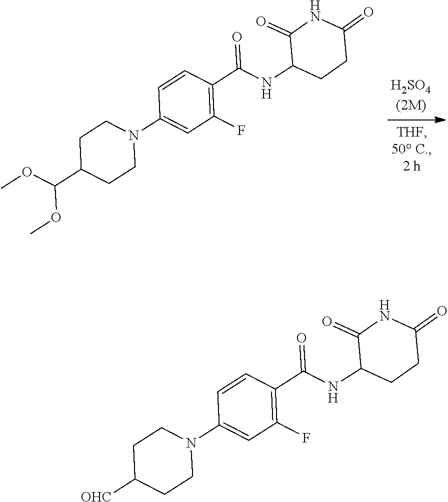 [SMILES: COC(OC)C1CCN(c2ccc(C(=O)NC3CCC(=O)NC3=O)c(F)c2)CC1 ; O=CC1CCN(c2ccc(C(=O)NC3CCC(=O)NC3=O)c(F)c2)CC1]

To a mixture of 4-[4-(dimethoxymethyl)-1-piperidyl]-N-(2,6-dioxo-3-piperidyl)-2-fluoro-benzamide (0.18 g, 0.44 mmol, 1 eq) in tetrahydrofuran (2 mL) was added sulfuric acid (2 M, 1 mL, 4.53 eq). The mixture was stirred at 50° C. for 2 h. The pH of the reaction mixture was adjusted to 7-8 with a saturated aqueous solution of sodium bicarbonate. The mixture was extracted with dichloromethane (10 mL×3). The combined organic layers were dried over anhydrous sodium sulfate, filtered, and concentrated under reduced pressure to give N-(2,6-dioxo-3-piperidyl)-2-fluoro-4-(4-formyl-1-piperidyl) benzamide (0.14 g, 0.38 mmol, 87% yield) as a white solid. MS (ESI) m/z: 362.1 [M+H]⁺.

### Step 5:

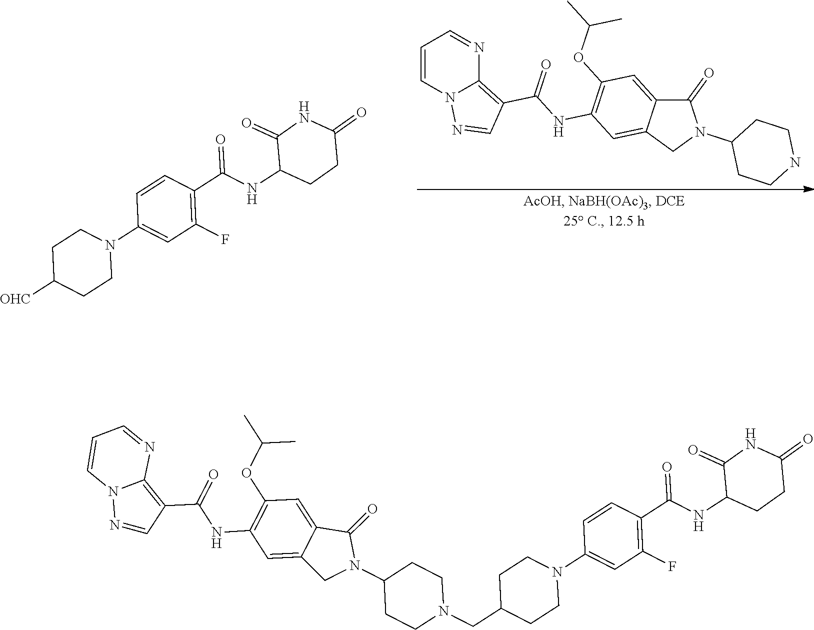 [SMILES: O=CC1CCN(c2ccc(C(=O)NC3CCC(=O)NC3=O)c(F)c2)CC1 ; CC(C)Oc1cc2c(cc1NC(=O)c1cnn3cccnc13)CN(C1CCNCC1)C2=O ; CC(C)Oc1cc2c(cc1NC(=O)c1cnn3cccnc13)CN(C1CCN(CC3CCN(c4ccc(C(=O)NC5CCC(=O)NC5=O)c(F)c4)CC3)CC1)C2=O]

To a mixture of N-[6-isopropoxy-1-oxo-2-(4-piperidyl)isoindolin-5-yl]pyrazolo[1,5-a]pyrimidine-3-carboxamide (0.16 g, 0.36 mmol, 1 eq) and N-(2,6-dioxo-3-piperidyl)-2-fluoro-4-(4-formyl-1-piperidyl)benzamide (133 mg, 0.36 mmol, 1 eq) in 1,2-dichloroethane (3 mL) was added acetic acid (22 mg, 0.36 mmol, 1 eq). The mixture was stirred at 25° C. for 0.5 h. Then sodium triacetoxyborohydride (234 mg, 1.10 mmol, 3 eq) was added to the mixture. The mixture was stirred at 25° C. for 12 h. The mixture was concentrated under reduced pressure to give a residue. The residue was purified by semi-preparative reverse phase HPLC (column: Venusil ASB Phenyl 150×30 mm×5 μm; mobile phase: [water (0.05% HCl)-acetonitrile]; B %: 25%-55%, 9 min) to give N-[2-[1-[[1-[4-[(2,6-dioxo-3-piperidyl)carbamoyl]-3-fluoro-phenyl]-4-piperidyl]methyl]-4-piperidyl]-6-isopropoxy-1-oxo-isoindolin-5-yl]pyrazolo[1,5-a]pyrimidine-3-carboxamide (15.5 mg, 0.02 mmol, MS yield, 99% purity, HCl) as a yellow solid. MS (ESI) m/z: 780.3 [M+H]⁺. ¹H NMR (400 MHz, DMSO-d₆) δ:10.93-10.67 (m, 2H), 10.01-9.79 (m, 1H), 9.46-9.32 (m, 1H), 8.97-8.66 (m, 3H), 8.12-7.93 (=, 1H), 7.70-7.52 (m, 1H), 7.44-7.27 (m, 2H), 6.90-6.72 (N, 2H), 4.97-4.22 (F, 4H), 4.02-3.75 (m, 2H), 3.27-2.66 (m, 9H), 2.40-1.81 (m, 11H), 1.55-1.36 (m, 6H), 1.34-1.15 (m, 2H).

TABLE 6
The following compounds may be prepared in an analogous fashion as
described for the synthesis of Example 286.

| | | ¹H NMR |
|---|---|---|
| | | All spectra acquired at 400 MHz |
| Example | MS | unless otherwise indicated |
| Example 286 | See Above | |
| 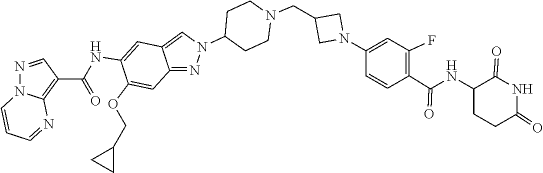 [SMILES: O=C1CCC(NC(=O)c2ccc(N3CC(CN4CCC(n5cc6cc(NC(=O)c7cnn8cccnc78)c(OCC7CC7)cc6n5)CC4)C3)cc2F)C(=O)N1] | 749.2 | MeOD δ = 10.83-10.79 (m, 1H), 9.10 (br d, J = 7.6 Hz, 1H), 8.83 (br s, 1H), 8.78 (s, 1H), 8.67 (s, 1H), 8.17 (s, 1H), 7.74-7.67 (m, 1H), 7.27-7.21 (m, 1H), 7.01 (s, 1H), 6.31-6.20 (m, 1H), 6.13 (br d, J = 14.4 Hz, 1H), 4.81-4.75 (m, 1H), 4.58-4.53 (m, 1H), 4.52- 4.44 (m, 1H), 4.15-4.09 (m, 2H), 4.07 (br d, J = 7.2 Hz, 2H), 3.72- 3.66 (m, 2H), 3.25-3.20 (m, 1H), 3.14-3.08 (m, 1H),3.00 (br d, J = |
| Example 287 | | 1.6 Hz, 2H), 2.70 (br d, J = 16.8 |
| | | Hz, 1H), 2.67-2.50 (m, 2H), 2.86- |
| | | 2.50(m, 1H), 2.36-2.11 (m, 6H), |
| | | 1.61-1.51 (m, 1H), 0.83 -0.74 (m, |
| | | 2H), 0.53 (br d, J = 2.8 Hz, 2H) |
| 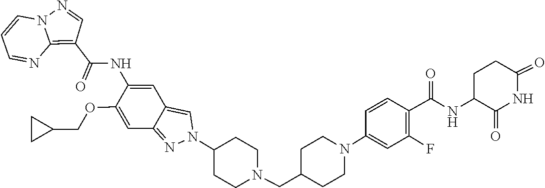 [SMILES: O=C1CCC(NC(=O)c2ccc(N3CCC(CN4CCC(n5cc6cc(NC(=O)c7cnn8cccnc78)c(OCC7CC7)cc6n5)CC4)CC3)cc2F)C(=O)N1] | 777.2 | DMSO-d₆ δ = 10.84 (s, 1 H) 10.66 (s, 1 H) 9.37 (dd, J = 7.0, 1.63 Hz, 1 H) 8.87 (dd, J = 4.2, 1.6 Hz, 1 H) 8.73-8.78 (m, 1 H) 8.71 (s, 1 H) 8.32 (s, 1 H) 8.13 (s, 1 H) 8.00 (t, J = 7.4 Hz, 1 H) 7.63 (t, J = 9.0 Hz, 1 H) 7.28-7.35 (m, 1 H) 7.03 (s, 1 H) 6.74-6.87 (m, 2 H) 4.68-4.79 (m, 1 H) 4.49 (br s, 1 H) 4.02 (d, J = 7.0 Hz, 2 H) 3.91 (br d, J = 13.2 Hz, 2 H) 2.83-2.90 (m, 2 H) 2.72- 2.82 (m, 2H) 2.51-2.58 (m, 4H) |
| Example 288 | | 1.93-2.29 (m, 8 H) 1.81 (br d, |
| | | J = 11.8 Hz, 3 H) 1.44-1.54 (m, 1 |
| | | H) 1.20 (br d, J = 12.6 Hz, 2H) 0.66- |
| | | 0.76 (m, 2 H) 0.46 (q, J = 4.6 Hz, 2H). |
| 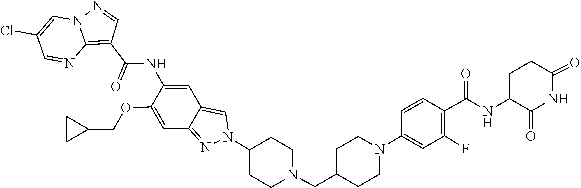 [SMILES: O=C1CCC(NC(=O)c2ccc(N3CCC(CN4CCC(n5cc6cc(NC(=O)c7cnn8cc(Cl)cnc78)c(OCC7CC7)cc6n5)CC4)CC3)cc2F)C(=O)N1] | 811.2 | DMSO-d₆ δ = 10.83 (s, 1 H), 10.42 (s, 1H), 9.82 (d, J = 2.2 Hz, 1H), 8.88 (d, J = 2.2 Hz, 1 H), 8.73 (d, J = 4.8 Hz, 2 H), 8.31 (s, 1H), 8.16 (s, 1 H), 7.99 (t, J = 7.2 Hz, 1H), 7.62 (t, J = 9.0 Hz, 1 H), 7.04 (s, 1 H), 6.86-6.69 (m, 2 H), 4.80-4.65 (m, 1 H), 4.37 (br s, 1 H), 4.02 (d, J = 7.2 Hz, 2 H), 3.89 (br d, J = 13.2 Hz, 2 H), 2.98 (br d, J = 4.8 Hz, 2 H), 2.90-2.70 (m, 4 H), 2.56- 2.53 (m, 1 H), 2.26-1.94 (m, 10 |
| Example 289 | | H), 1.87-1.74 (m, 3 H), 1.51 (ddd, |
| | | J = 5.2, 7.6, 12.4 Hz, 1 H), 1.21- |
| | | 1.06 (m, 2 H), 0.75-0.63 (m, 2 H), |
| | | 0.49-0.38 (m, 2 H) |
| 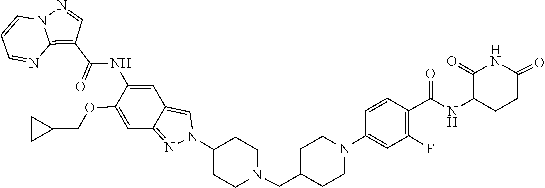 [SMILES: O=C1CCC(NC(=O)c2ccc(N3CCC(CN4CCC(n5cc6cc(NC(=O)c7cnn8cccnc78)c(OCC7CC7)cc6n5)CC4)CC3)cc2F)C(=O)N1] | 724.2 | DMSO-d₆ δ = 10.86 (s, 1 H) 10.65 (s, 1 H) 9.38 (dd, J = 7.2, 1.6 Hz, 1 H) 8.87 (dd, J = 4.0, 1.6 Hz, 1 H) 8.73 (d, J = 10.4 Hz, 2 H) 8.28-8.35 (m, 2 H) 7.69 (t, J = 9.2 Hz, 1 H) 7.29-7.35 (m, 1 H) 7.04 (s, 1 H) 6.98 (dd, J = 13.2, 2.4 Hz, 1 H) 6.92 (dd, J = 8.8, 2.4 Hz, 1 H) 4.68- 4.81 (m, 1 H) 4.32-4.45 (m, 1 H) 4.20 (t, J = 6.4 Hz, 2 H) 4.02 (d, J = 6.8Hz, 2 H) 3.09 (d, J = 11.2 Hz, 2 H) 2.75-2.85 (m, 3 H) 2.54-2.53 |
| Example 290 | | (m, 1 H) 2.23-2.36 (m, 2 H) 1.96-2.15 |
| | | (m, 6 H) 1.42-1.57 (m, 1 H) 0.66- |
| | | 0.75 (m, 2 H) 0.45-0.44 (m, 2 H) |
| 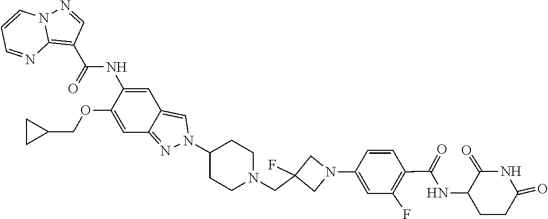 [SMILES: O=C1CCC(NC(=O)c2ccc(N3CC(F)(CN4CCC(n5cc6cc(NC(=O)c7cnn8cccnc78)c(OCC7CC7)cc6n5)CC4)C3)cc2F)C(=O)N1] | 767.2 | DMSO-d₆ δ = 10.84 (s, 1H), 10.65 (s, 1H), 9.37 (d, J = 6.8 Hz, 1H), 8.92-8.83 (m, 1H), 8.72 (d, J = 11.6 Hz, 2H), 8.30 (s, 1H), 8.12- 7.97 (m, 1H), 7.64 (t, J = 8.4 Hz, 1H), 7.38-7.26 (m, 1H), 7.03 (s, 1H), 6.47-6.33 (m, 2H), 4.84-4.67 (m, 1H), 4.46-4.29 (m, 1H), 4.17-3.99 (m, 6H), 3.09-3.01 (m, 2H), 2.97-2.89 (m, 2H), 2.82-2.70 (m, 2H), 2.17-1.96 (m, 6H), 1.58-1.46 (m, 1H), 1.29-1.16 (m,1H), 0.90-0.79 (m, 1H), 0.78- 0.64 (m, 2H), 0.52-0.40 (m, 2H) |
| Example 291 | | |
| 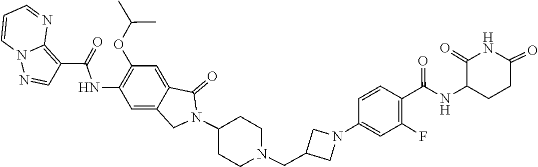 [SMILES: CC(C)Oc1cc2c(cc1NC(=O)c1cnn3cccnc13)CN(C1CCN(CC3CN(c4ccc(C(=O)NC5CCC(=O)NC5=O)c(F)c4)C3)CC1)C2=O] | 752.3 | DMSO-d₆ δ = 10.84 (s, 1 H) 10.75 (s, 1 H) 9.39 (dd, J = 6.8, 1.6 Hz, 1 H) 8.89 (dd, J = 4.4, 1.6 Hz, 1 H) 8.72-8.76 (m, 2 H) 7.96 (m, 1 H) 7.61 (t, J = 8.4 Hz, 1 H) 7.34-7.38 (m, 1 H) 7.33 (s, 1 H) 6.19-6.30 (m, 2 H) 4.90 (m, 1 H) 4.68-4.79 (m, 1 H) 4.42 (s, 2 H) 3.94-4.04 (m, 3 H) 3.57 (dd, J = 7.2, 5.6 Hz, 2 H)3.31 (br s, 1H) 2.91-3.00 (m, 3 H) 2.71-2.81 (m, 1 H) 2.61 (m, |
| Example 292 | | 2 H) 2.51-2.57 (m, 2 H) 1.97-2.12 |
| | | (m, 4 H) 1.73-1.86 (m, 2 H) 1.64-1.72 |
| | | (m, 2 H) 1.44 (d, J = 6.0 Hz, 6 H) |
| 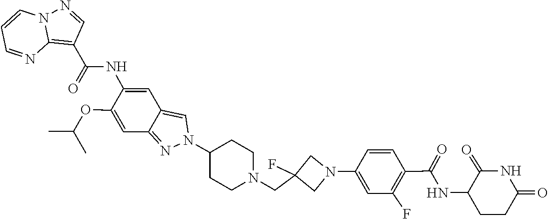 [SMILES: CC(C)Oc1cc2nn(C3CCN(CC4(F)CN(c5ccc(C(=O)NC6CCC(=O)NC6=O)c(F)c5)C4)CC3)cc2cc1NC(=O)c1cnn2cccnc12] | 755.3 | DMSO-d₆ δ = 10.85 (s, 1 H), 10.60 (s, 1H), 9.39 (d, J = 5.6 Hz, 1 H), 8.87 (d, J = 2.4 Hz, 1 H), 8.75 (s, 1 H), 8.71 (s, 1 H), 8.31 (s, 1 H), 8.13 (s, 1 H), 8.05 (s, 1 H), 7.64 (s, 1 H), 7.34 (dd, J = 6.8, 4.4 Hz, 1 H), 7.13 (s, 1H), 6.40 (d, J = 6.8 Hz, 2 H), 4.86 (s, 1 H), 4.70-4.77 (m, 1 H), 4.38 (s, 1 H), 3.97-4.16 (m, 3 H), 3.07 (s, 1 H), 2.90- 2.98 (m, 1 H), 2.70-2.85 (m, 1 H), 2.54 (s, 2 H), 2.52-2.52 (m, 2 H), 2.41 (s, 2 H), 2.05-2.15 (m, 4 H), 2.00 (s, 1 H), 1.47 (d, J = 6.0 Hz, 6 H), 1.24-1.27 (m, 1 H). |
| Example 293 | | |
| 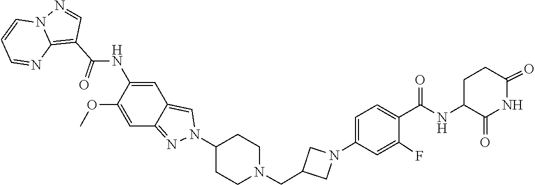 [SMILES: COc1cc2nn(C3CCN(CC4CN(c5ccc(C(=O)NC6CCC(=O)NC6=O)c(F)c5)C4)CC3)cc2cc1NC(=O)c1cnn2cccnc12] | 709.2 | MeOD δ = 9.11 (br d, J = 6.8 Hz, 1H), 8.88 (br d, J = 4.4 Hz, 1H), 8.75-8.65 (m, 2H), 8.18 (s, 1H), 7.72 (br t, J = 7.6 Hz, 1H), 7.25 (br d, J = 4.8 Hz, 1H), 7.07 (d, J = 1.6 Hz, 1H), 6.30-6.24 (m, 1H), 6.15 (br d, J = 12.8 Hz, 1H), 4.56 (s, 1H), 4.44 (s, 1H), 4.14-4.08 (m, 5H), 3.76 (s, 1H), 3.71-3.63 (m, 2H), 3.17-3.05 (m, 3H), 2.88- 2.80 (m, 2H), 2.80-2.65 (m, 1H), 2.46-2.20 (m, 8H) |
| Example 294 | | |
| 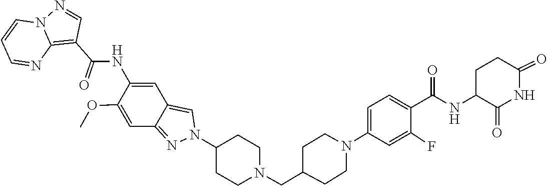 [SMILES: COc1cc2nn(C3CCN(CC4CCN(c5ccc(C(=O)NC6CCC(=O)NC6=O)c(F)c5)CC4)CC3)cc2cc1NC(=O)c1cnn2cccnc12] | 737.2 | DMSO-d₆ δ = 10.84 (s, 1 H) 10.54 (s, 1 H) 9.38 (dd, J = 7.0, 1.5 Hz, 1 H) 8.95 (dd, J = 4.2, 1.6 Hz, 1 H) 8.69 (d, J = 7.8 Hz, 2 H) 8.32 (s, 1 H) 8.14 (s, 1 H) 8.00 (br 1, J = 7.3 Hz, 1 H) 7.62 (t, J = 9.2 Hz, 1 H) 7.35 (dd, J = 7.0, 4.2 Hz, 1 H) 7.10 (s, 1 H) 6.71-6.87 (m, 2 H) 4.68- 4.79 (m, 1 H) 4.39 (br s, 1 H) 4.03 (s, 3 H) 3.89 (br d, J = 12.2 Hz, 2H) 3.01 (br s, 2 H) 2.71-2.90 (m, 4 H) 2.26 (br s, 2 H) 2.12 (br d, J = 9.0 |
| Example 295 | | Hz, 4 H) 2.02 (br s, 1 H) 1.80 (br |
| | | d, J = 10.6 Hz, 3 H) 1.16 (br d, |
| | | J = 12.8 Hz, 2H). |
| 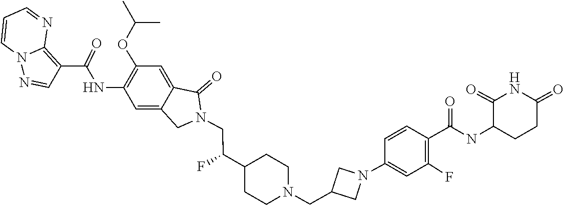 | 764.3 | DMSO-d₆ δ = 10.83 (s, 1 H), 10.76 (s, 1H), 9.41 (dd, J = 7.2, 1.6 Hz, 1 H), 8.90 (dd, J = 4.4, 1.6 Hz, 1 H), 8.74 (s, 2 H), 7.96 (1, J = 7.6 Hz, 1 H), 7.60 (t, J = 8.8 Hz, 1 H), 7.33-7.39 (m, 2 H), 6.26 (dd, J = 8.8, 2.0 Hz, 1 H), 6.21 (dd, J = 14.0, 2.0 Hz, 1 H), 4.91 (dt, J = 12.0, 6.0 Hz, 1 H), 4.67-4.75 (m, 1 H), 4.50 (d, J = 8.0 Hz, 2 H), 3.99 (t, J = 8.0 Hz, 2 H), 3.68-3.86 (m, 2 H), 3.51-3.57 (m, 2 H), 2.91-2.98 (m, 1 H), 2.87 (d, J = 9.6 Hz, 2 H), |
| Example 296 | | 2.72-2.81 (m, 1 H), 2.52-2.56 (m, |
| | | 3 H), 2.06-2.15 (m, 1 H), 1.98-2.03 |
| | | (m, 1H), 1.90(t, J = 11.2 Hz, 2 H), |
| | | 1.67-1.82 (m, 2 H), 1.56 (s, 1 H), |
| | | 1.45 (d, J = 6.0 Hz, 6 H), 1.30-1.39 |
| | | (m, 2H), 1.23 (s, 1 H). |
| 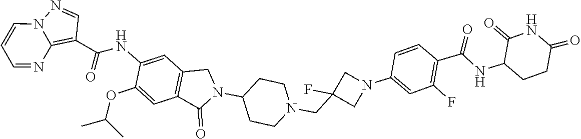 [SMILES: CC(C)Oc1cc2c(cc1NC(=O)c1cnn3cccnc13)CN(C1CCN(CC3(F)CN(c4ccc(C(=O)NC5CCC(=O)NC5=O)c(F)c4)C3)CC1)C2=O] | 770.3 | DMSO-d₆ δ = 10.84 (s, 1H), 10.76 (s, 1H), 9.40 (dd, J = 1.6, 7.2 Hz, 1H), 8.89 (dd, J = 1.6, 4.4 Hz, 1H), 8.80-8.70 (m, 2H), 8.10-8.01 (m, 1H), 7.71-7.59 (m, 1H), 7.437.28- (m, 2H), 6.46-6.31 (m, 2H), 4.98-4.84 (m, 1H), 4.79-4.69 (m, 1H), 4.42 (s, 2H), 4.32-3.92 (m, 5H), 3.65-3.37 (m, 3H), 3.09-2.71 |
| Example 297 | | (m, 3H), 2.59-2.52 (m, 2H), 2.37-2.24 |
| | | (m, 1H), 2.19 -1.94 (m, 3H), 1.86-1.63 |
| | | (m, 2H), 1.44 (d, J = 6.0 Hz, 6H) |
| 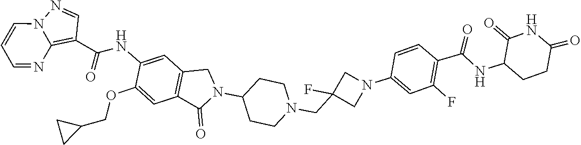 [SMILES: O=C1CCC(NC(=O)c2ccc(N3CC(F)(CN4CCC(N5Cc6cc(NC(=O)c7cnn8cccnc78)c(OCC7CC7)cc6C5=O)CC4)C3)cc2F)C(=O)N1] | 782.3 | DMSO-d₆ δ = 10.83 (d, J =9.6 Hz, 2H), 9.40 (dd, J = 1.6, 7.2 Hz, 1H), 8.89 (dd, J = 1.6, 4.4 Hz, 1H), 8.78-8.69 (m, 2H), 8.05 (br t, J = 6.8 Hz, 1H), 7.65 (br t, J = 8.8 Hz, 1H), 7.34 (dd, J = 4.4, 7.2 Hz, 1H), 7.26 (s, 1H), 6.43-6.34 (m, 2H), 4.80-4.71 (m, 1H), 4.40 (br s, 2H), 4.21-3.94 (m, 7H), 3.16-2.96 (m, 2H), 2.96-2.70 (m, 3H), 2.53 (br d, |
| Example 298 | | J = 6.8 Hz, 2H), 2.32-2.20 (m, |
| | | 1H), 2.12 (dq, J = 4.4, 12.8 Hz, |
| | | 1H), 2.05-1.98 (m, 1H), 1.97-1.53 |
| | | (m, 4H), 1.51-1.40 (m, 1H), 0.73- |
| | | 0.65 (m, 2H), 0.45 (q, J = 4.4 Hz, 2H). |
| 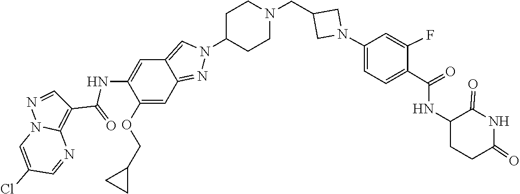 [SMILES: O=C1CCC(NC(=O)c2ccc(N3CC(CN4CCC(n5cc6cc(NC(=O)c7cnn8cc(Cl)cnc78)c(OCC7CC7)cc6n5)CC4)C3)cc2F)C(=O)N1] | 783.2 | DMSO-d₆ δ = 10.84 (s, 1H), 10.43 (s, 1H), 9.83 (d, J = 2.0 Hz, 1H), 8.89 (d, J = 2.4 Hz, 1H), 8.74 (s, 2H), 8.32 (s, 1H), 8.13 (s, 1H), 7.98 (t, J = 7.2 Hz, 1H), 7.63 (t, J = 8.8 Hz, 1H), 7.05 (s, 1H), 6.32-6.27 (m, 1H), 6.25 (br d, J = 14.0 Hz, 1H), 4.76-4.68 (m, 1H), 4.58-4.27 (m, 1H), 4.09-4.00 (m, 4H), 3.67-3.49 (m, 3H), 3.28-2.97 (m, 6H), 2.83-2.71 (m, 2H), 2.22-1.94 (m, 6H), 1.58-1.46 (m, 1H), 0.73-0.67 (m, 2H), 0.43 (q, J = 5.2 Hz, 2H) |
| Example 299 | | |
| 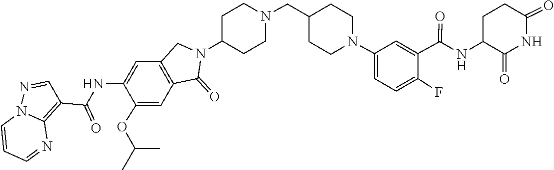 [SMILES: CC(C)Oc1cc2c(cc1NC(=O)c1cnn3cccnc13)CN(C1CCN(CC3CCN(c4ccc(F)c(C(=O)NC5CCC(=O)NC5=O)c4)CC3)CC1)C2=O] | 780.3 | DMSO-d₆ δ = 10.88 (s, 1H), 10.76 (s, 1H), 9.41 (dd, J = 1.6, 7.2 Hz, 1H), 8.90 (dd, J = 1.6, 4.4 Hz, 1H), 8.74 (s, 2H), 8.49 (dd, J = 3.2, 8.0 Hz, 1H), 8.20 (s, 1H), 7.36 (dd, J = 4.4, 7.2 Hz, 1H), 7.34 (s, 1H), 7.18-7.08 (m, 3H), 4.90 (td, J = 6.0, 12.0 Hz, 1H), 4.80-4.72 (m, 1H), 4.43 (s, 2H), 4.04-3.96 (m, 1H), 3.63 (br d, J = 12.0 Hz, 2H), 2.96 (br d, J = 11.2 Hz, 2H), 2.84-2.75 (m, 1H), 2.66 (br t, J = 10.8 |
| Example 303 | | Hz, 2H), 2.55 (br d, J = 4.8 Hz, |
| | | 1H), 2.21 (br d, J = 7.2 Hz, 2H), |
| | | 2.17-2.07 (m, 1H), 2.06-1.98 (m, |
| | | 3H), 1.86-1.76 (m, 4H), 1.69 (br d, |
| | | J = 8.4 Hz, 3H), 1.44 (d, J = 6.0 Hz, |
| | | 6H), 1.28-1.16 (m, 2H) |
| 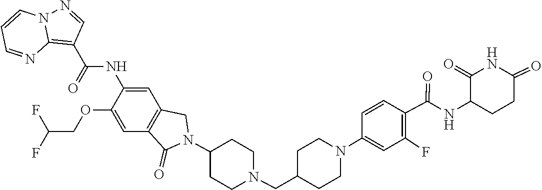 [SMILES: O=C1CCC(NC(=O)c2ccc(N3CCC(CN4CCC(N5Cc6cc(NC(=O)c7cnn8cccnc78)c(OCC(F)F)cc6C5=O)CC4)CC3)cc2F)C(=O)N1] | 802.2 | DMSO-d₆ δ = 10.84 (s, 1H), 10.77 (s, 1H), 9.40 (dd, J = 1.2, 6.8 Hz, 1H), 8.86 (dd, J = 1.6, 4.4 Hz, 1H), 8.75 (d, J = 7.2 Hz, 2H), 8.00 (t, J = 7.2 Hz, 1H), 7.62 (t, J = 9.2 Hz, 1H), 7.44-7.33 (m, 2H), 6.88-6.70 (m, 2H), 6.64-6.41 (m, 1H), 4.79-4.68 (m, 1H), 4.60 (t, J = 3.2, 14.4 Hz, 2H), 4.44 (s, 2H), 4.07-3.95 (m, 1H), 3.88 (d, J = 12.8 Hz, 2H), 2.95 (d, J = 10.4 Hz, 2H), 2.88-2.71 (m, 4H), 2.22-2.10 (m, 3H), 2.06-1.95 (m, 3H), 1.83-1.65 (m, |
| Example 305 | | 7H), 1.23-1.08 (m, 2H) |
| 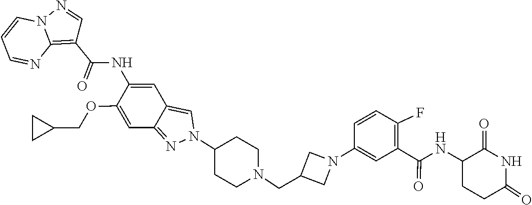 [SMILES: O=C1CCC(NC(=O)c2cc(N3CC(CN4CCC(n5cc6cc(NC(=O)c7cnn8cccnc78)c(OCC7CC7)cc6n5)CC4)C3)ccc2F)C(=O)N1] | 749.3 | DMSO-d₆ δ 10.87 (br s, 1H), 10.66 (s, 1H), 9.38 (dd, J = 1.6, 7.2 Hz, 1H), 8.87 (dd, J = 1.6, 4.4 Hz, 1H), 8.73 (d, J = 10.4 Hz, 2H), 8.45 (dd, J = 3.6, 8.0 Hz, 1H), 8.34 (s, 1H), 8.31 (s, 1H), 7.33 (dd, J = 4.4, 7.2 Hz, 1H), 7.17- 7.08 (m, 1H), 7.04 (s, 1H), 6.64 (dd, J = 3.2, 6.0 Hz, 1H), 6.57 (td, J = 3.6, 8.8 Hz, 1H), 4.80-4.71 (m, 1H), 4.36 (td, J = 8.0, 15.2 Hz, 1H), 4.03 (d, J = 6.8 Hz, 2H), 3.97 (t, J = 7.2 Hz, 2H), 3.50 (br d, J = 6.4 Hz, 2H), 2.97 (br d, J = 11.6 Hz, 3H), 2.84-2.74 (m, 1H), |
| Example 306 | | 2.64 (br d, J = 7.2 Hz, 2H), 2.55 (s, |
| | | 1H),2.22-2.14 (m, 2H), 2.13-2.05 |
| | | (m, 5H), 2.04-1.99 (m, 1H), 1.55-1.47 |
| | | (m, 1H), 0.74-0.69 (m, 2H), 0.49-0.44 |
| | | (m, 2H) |
| 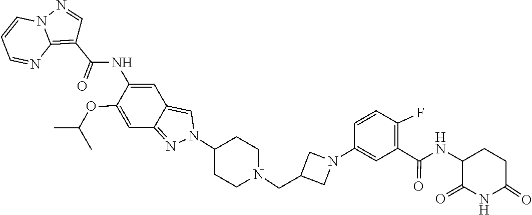 [SMILES: CC(C)Oc1cc2nn(C3CCN(CC4CN(c5ccc(F)c(C(=O)NC6CCC(=O)NC6=O)c5)C4)CC3)cc2cc1NC(=O)c1cnn2cccnc12] | 752.2 | DMSO-d₆ δ = 10.87 (s, 1H), 10.77 (s, 1H), 9.41 (dd, J = 1.6, 6.8 Hz, 1H), 8.90 (dd, J = 1.6, 4.0 Hz, 1H), 8.75 (s, 2H), 8.44 (dd, J = 3.6, 8.0 Hz, 1H), 8.26 (s, 1H), 7.37 (dd, J = 4.4, 6.8 Hz, 1H), 7.34 (s, 1H), 7.15-7.08 (m, 1H), 6.64 (dd, J = 3.2, 6.0 Hz, 1H), 6.60-6.54 (m, 1H), 4.91 (td, J = 6.0, 12.0 Hz, 1H), 4.79-4.72 (m, 1H), 4.43 (s, 2H), 4.05-3.98 (m, 1H), 3.96 (br t, J = 7.2 Hz, 2H), 3.51-3.47 (m, 2H), 2.94 (br d, J = 8.4 Hz, 3H), 2.84-2.75 (m, 1H), 2.61 (br d, J = 7.2 Hz, 2H), |
| Example 307 | | 2.55 (br d, J = 3.6 Hz, |
| | | 1H), 2.14-2.05 (m, 3H), 2.04-1.98 |
| | | (m, 1H), 1.85-1.75 (m, 2H), 1.73- |
| | | 1.66 (m, 2H), 1.45 (d, J = 6.0 Hz, 6H) |
| 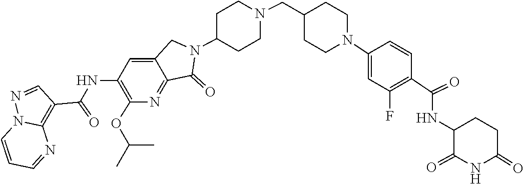 [SMILES: CC(C)Oc1nc2c(cc1NC(=O)c1cnn3cccnc13)CN(C1CCN(CC3CCN(c4ccc(C(=O)NC5CCC(=O)NC5=O)c(F)c4)CC3)CC1)C2=O] | 781.3 | DMSO-d₆ δ = 10.84 (s, 1H), 10.77 (br s, 1H), 9.45-9.39 (m, 1H), 8.93 (br d, J = 8.0 Hz, 2H), 8.78- 8.73 (m, 1H), 8.19 (s, 1H), 8.00 (t, J = 7.2 Hz, 1H), 7.66-7.59 (m, 1H), 7.37 (br dd, J = 2.6, 6.8 Hz, 1H), 6.84-6.70 (m, 2H), 5.43 (td, J = 5.8, 12.0 Hz, 1H), 4.78-4.67 (m, 1H), 4.42 (br s, 2H), 4.01 (br t, J = 10.6 Hz, 1H), 3.89 (br d, J = 12.0 Hz, 2H), 3.26 (br d, J = 6.4 Hz, 1H), 2.95 (br d, J = 10.6 Hz, |
| Example 309 | | 2H), 2.88-2.71 (m, 3H), 2.18 (br |
| | | d, J = 6.0 Hz, 2H), 2.15-1.97 (m, |
| | | 4H), 1.83-1.67 (m, 7H), 1.49 (d, J = |
| | | 6.2 Hz, 6H), 1.20-1.09 (m, 2H) |
| 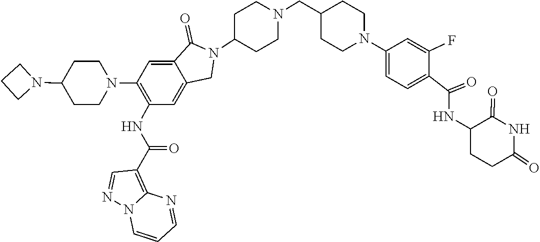 [SMILES: O=C1CCC(NC(=O)c2ccc(N3CCC(CN4CCC(N5Cc6cc(NC(=O)c7cnn8cccnc78)c(N7CCC(N8CCC8)CC7)cc6C5=O)CC4)CC3)cc2F)C(=O)N1] | 860.4 | DMSO-d₆ δ = 10.84 (s, 1H), 10.77 (br s, 1H), 9.45-9.39 (m, 1H), 8.93 (br d, J = 8.0 Hz, 2H), 8.78-8.73 (m, 1H), 8.19 (s, 1H), 8.00 (t, J = 7.2 Hz, 1H), 7.66-7.59 (m, 1H), 7.37 (br dd, J = 2.6, 6.8 Hz, 1H), 6.84-6.70 (m, 2H), 5.43 (td, J = 5.8, 12.0 Hz, 1H), 4.78-4.67 (m, 1H), 4.42 (br s, 2H), 4.01 (br t, J = 10.6 Hz, 1H), 3.89 (br d, J = 12.0 Hz, 2H), 3.26 (br d, J = 6.4 Hz, 1H), 2.95 (br d, J = 10.6 Hz, 2H), 2.88-2.71 (m, 3H), 2.18 (br d, J = 6.0 Hz, 2H), 2.15-1.97 (m, 4H), 1.83-1.67 (m, 7H), 1.49 (d, J = 6.2 Hz, 6H), 1.20-1.09 (m, 2H) |
| Example 310 | | |
| 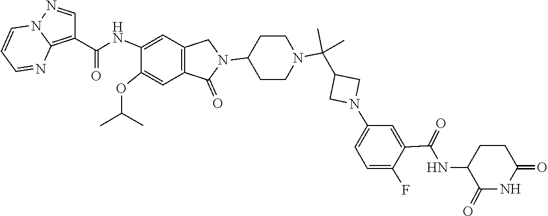 [SMILES: CC(C)Oc1cc2c(cc1NC(=O)c1cnn3cccnc13)CN(C1CCN(C(C)(C)C3CN(c4ccc(F)c(C(=O)NC5CCC(=O)NC5=O)c4)C3)CC1)C2=O] | 780.3 | DMSO-d₆ δ = 10.86 (s, 1H), 10.77 (s, 1H), 9.41 (dd, J = 1.6, 6.8 Hz, 1H), 8.90 (dd, J = 1.6, 4.4 Hz, 1H), 8.77 (s, 1H), 8.73 (s, 1H), 8.44 (dd, J = 4.0, 8.4 Hz, 1H), 7.39-7.34 (m, 2H), 7.14 (br t, J = 9.2 Hz, 1H), 6.67 (br d, J = 2.4 Hz, 1H), 6.62-6.56 (m, 1H), 4.95-4.87 (m, 1H), 4.79-4.70 (m, 1H), 4.43 (s, 2H), 3.99-3.89 (m, 2H), 3.65 (br s, 2H), 3.26-3.15 (m, 2H), 2.84-2.74 (m, 2H), 2.55-2.54 (m, 2H), 2.21-1.71 (m, 8H), 1.44 (d, J = 6.0 Hz, 6H), 1.35-1.07 (m, 6H) |
| Example 311 | | |
| 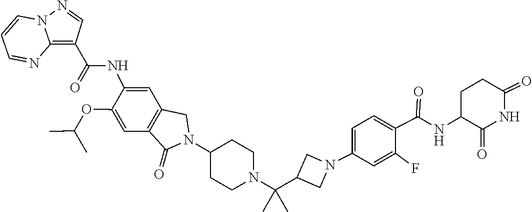 [SMILES: CC(C)Oc1cc2c(cc1NC(=O)c1cnn3cccnc13)CN(C1CCN(C(C)(C)C3CN(c4ccc(C(=O)NC5CCC(=O)NC5=O)c(F)c4)C3)CC1)C2=O] | 780.3 | DMSO-d₆ δ = 10.85 (s, 1H), 10.78 (s, 1H), 9.42 (dd, J = 1.6, 7.2 Hz, 1H), 8.91 (dd, J = 1.6, 4.0 Hz, 1H), 8.79 (s, 1H), 8.74 (s, 1H), 8.01 (br t, J = 7.2 Hz, 1H), 7.65 (1, J = 8.8 Hz, 1H), 7.41-7.34 (m, 2H), 6.35-6.21 (m, 2H), 4.92 (td, J = 6.0,12.0 Hz, 1H), 4.78-4.69 (m, 1H), 4.45 (s, 2H), 4.37-4.20 (m, 1H), 4.04 (br t, J = 7.6 Hz, 2H), 3.81 (br t, J = 6.8 Hz, 2H), 3.47-3.41 (m, 1H), 3.10-2.71 (m, 3H), 2.60-2.53 (m, 2H), 2.49-2.44 (m, 1H), 2.19-2.04 (m, 2H), 2.04-1.97 (m, 2H), 1.97- 1.88 (m, 2H), 1.45 (d, J = 6.0 Hz, 6H), |
| Example 312 | | 1.29 (br s, 6H) |

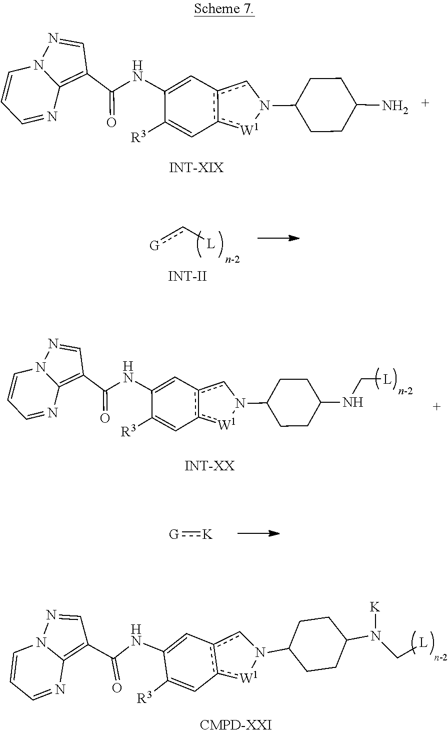

A compound of formula INT-XIX, wherein R³ is an ether, an amine, or an alkyl group and W¹ is optionally N or C═O, can be prepared through substitution of 4-amino-1-Boc-piperidine in step 5 or step 4 of Scheme 1C or Scheme 3A, respectively, with either trans or cis N-Boc-1,4-cyclohexanediamine. A compound of formula INT-XIX may be reacted with a reagent INT-II (commercially available or readily prepared using standard reaction techniques known to one skilled in the art) under reductive amination or alkylation conditions to produce a compound of formula INT-XX, wherein L and n are as defined herein. Compounds of formula INT-XX can be furnished from N-alkylation where G is an appropriate leaving group (e.g., OMs, OTs, Cl, etc.) or through reductive amination where G is an aldehyde or ketone. When G is a leaving group, suitable reaction conditions are those for an alkylation reaction, e.g., diisopropylethylamine, potassium iodide, DMSO or acetonitrile, 80° C. When G is an aldehyde, suitable reaction conditions are those for a reductive amination reaction, e.g., sodium cyanoborohydride, methanol, dichloromethane, acetic acid, room temperature. Treatment of compounds of formula INT-XX with a suitable reagent for alkylation (either by S_{N}2 or reductive amination) of the secondary amine allow for the preparation of synthetic intermediates of the general formula CMPD-XXI. These intermediates can be reacted further as detailed in Schemes 1-6.

### Preparation of Example 313: N-(2-((1r,4r)-4-((1-(2-(2,6-dioxopiperidin-3-yl)-1,3-dioxoisoindolin-5-yl)azetidin-3-yl)(methyl)amino)cyclohexyl)-6-isopropoxy-1-oxoisoindolin-5-yl)pyrazolo[1,5-a]pyrimidine-3-carboxamide

### Step 1:

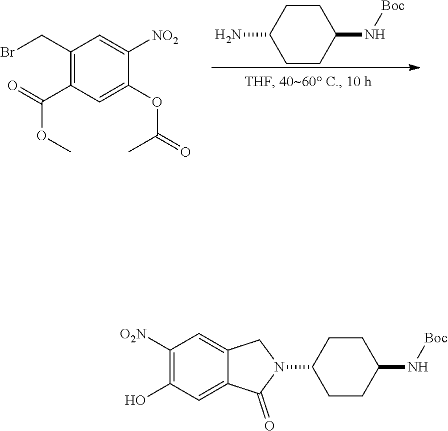 [SMILES: COC(=O)c1cc(OC(C)=O)c([N+](=O)[O-])cc1CBr ; CC(C)(C)OC(=O)N[C@H]1CC[C@H](N)CC1 ; CC(C)(C)OC(=O)N[C@H]1CC[C@H](N2Cc3cc([N+](=O)[O-])c(O)cc3C2=O)CC1]

To a solution of tert-butyl N-(4-aminocyclohexyl) carbamate (2 g, 9.33 mmol, 3.10 eq) in tetrahydrofuran (20 mL) was added diisopropylethylamine (778.3 mg, 6.02 mmol, 1.05 mL, 2 eq), followed by the slow addition of methyl 5-acetoxy-2-(bromomethyl)-4-nitro-benzoate (1 g, 3.01 mmol, 1 eq) in tetrahydrofuran (10 mL) over a period of 30 min at 40° C. The mixture was stirred at 60° C. for 10 h. The reaction mixture was quenched by the addition of water (20 mL) at 0° C., the aqueous phase was acidified with acetic acid until the pH=7, and then diluted with water (20 mL) and extracted with ethyl acetate (20 mL×3). The combined organic layers were washed with brine (20 mL×3), dried over anhydrous sodium sulfate, filtered, and concentrated under reduced pressure to give a residue. The residue was purified by column chromatography (silicon dioxide, eluted with petroleum ether/ethyl acetate=5/1 to 1/1) to afford tert-butyl N-[4-(6-hydroxy-5-nitro-1-oxo-isoindolin-2-yl)cyclohexyl]carbamate (380 mg, 970.8 μmol, 32% yield) as a yellow solid. MS (ESI) m/z: 414.1[M+Na]⁺.

### Step 2:

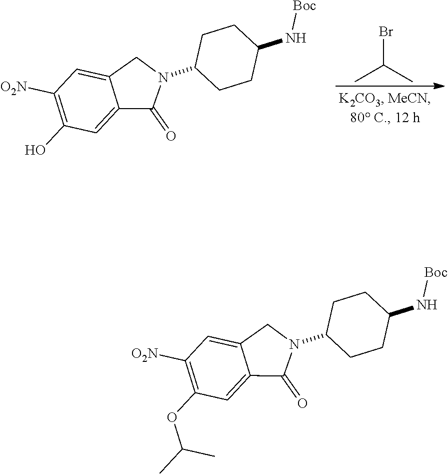

To a solution of tert-butyl N-[4-(6-hydroxy-5-nitro-1-oxo-isoindolin-2-yl)cyclohexyl]carbamate (750 mg, 1.92 mmol, 1 eq) in N,N-dimethylformamide (5 mL) was added potassium carbonate (1.32 g, 9.58 mmol, 5 eq) and 2-bromopropane (6.55 g, 53.3 mmol, 5 mL, 27.79 eq). The mixture was stirred at 80° C. for 12 h. The reaction mixture was partitioned between water (50 mL) and ethyl acetate (100 mL×3). The combined organic phase was separated, washed with brine (100 mL×3), dried over anhydrous sodium sulfate, filtered, and concentrated under reduced pressure to give a residue. The residue was purified by column chromatography (silicon dioxide, eluted with petroleum ether/ethyl acetate=5/1 to 1/1) to afford tert-butyl N-[4-(6-isopropoxy-5-nitro-1-oxo-isoindolin-2-yl)cyclohexyl]carbamate (620 mg, 1.43 mmol, 74% yield) as a white solid. MS (ESI) m/z: 456.3[M+Na]⁺.

### Step 3:

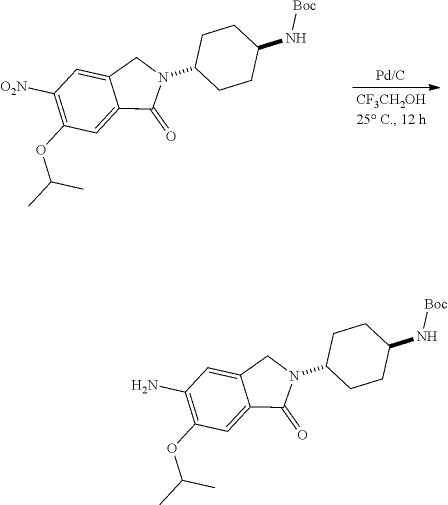 [SMILES: CC(C)Oc1cc2c(cc1[N+](=O)[O-])CN([C@H]1CC[C@H](NC(=O)OC(C)(C)C)CC1)C2=O ; CC(C)Oc1cc2c(cc1N)CN([C@H]1CC[C@H](NC(=O)OC(C)(C)C)CC1)C2=O]

To a solution of tert-butyl N-[4-(6-isopropoxy-5-nitro-1-oxo-isoindolin-2-yl) cyclohexyl]carbamate (620 mg, 1.43 mmol, 1 eq) in trifluoroethanol (10 mL) was added palladium on carbon (10 mg, 143.02 μmol, 10% purity, 0.1 eq) under nitrogen atmosphere. The suspension was degassed under vacuum and purged with hydrogen over several cycles. The mixture was stirred under hydrogen (15 psi) at 25° C. for 0.5 h. The reaction mixture was filtered, and the filtrate was concentrated. The crude product, tert-butyl N-[4-(5-amino-6-isopropoxy-1-oxo-isoindolin-2-yl) cyclohexyl]carbamate (570 mg, 1.41 mmol, 98% yield), was obtained as a white solid and was used into the next step without further purification. MS (ESI) m/z: 404.3[M+H]⁺.

### Step 4:

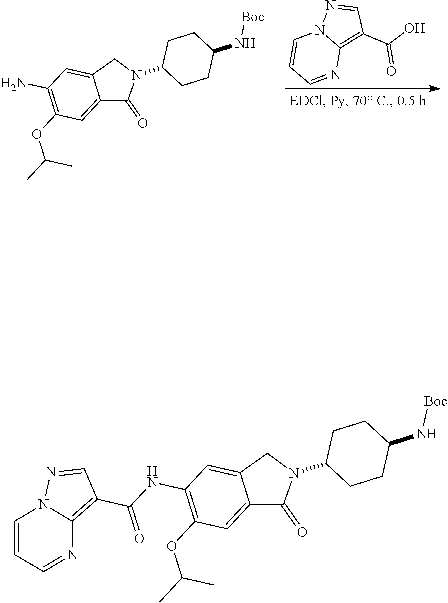 [SMILES: CC(C)Oc1cc2c(cc1N)CN([C@H]1CC[C@H](NC(=O)OC(C)(C)C)CC1)C2=O ; O=C(O)c1cnn2cccnc12 ; CC(C)Oc1cc2c(cc1NC(=O)c1cnn3cccnc13)CN([C@H]1CC[C@H](NC(=O)OC(C)(C)C)CC1)C2=O]

To a solution of tert-butyl N-[4-(5-amino-6-isopropoxy-1-oxo-isoindolin-2-yl) cyclohexyl]carbamate (570 mg, 1.41 mmol, 1 eq) in pyridine (10 mL) was added 1-ethyl-3-(3-dimethylamino-propyl)-carbodiimide hydrochloride (1.10 g, 5.72 mmol, 4.05 eq) and pyrazolo[1,5-a]pyrimidine-3-carboxylic acid (230.44 mg, 1.41 mmol, 1 eq). The mixture was stirred at 70° C. for 0.5 h. The reaction mixture was concentrated under reduced pressure and the resulting residue was purified by column chromatography (silicon dioxide, eluted with petroleum ether/ethyl acetate=1/1 to 0/1) to afford tert-butyl N-[4-[6-isopropoxy-1-oxo-5-(pyrazolo[1,5-a]pyrimidine-3-carbonylamino)isoindolin-2-yl]cyclohexyl]carbamate (570 mg, 1.04 mmol, 73.5% yield) as a yellow solid. MS (ESI) m/z: 549.3[M+H]⁺.

### Step 5:

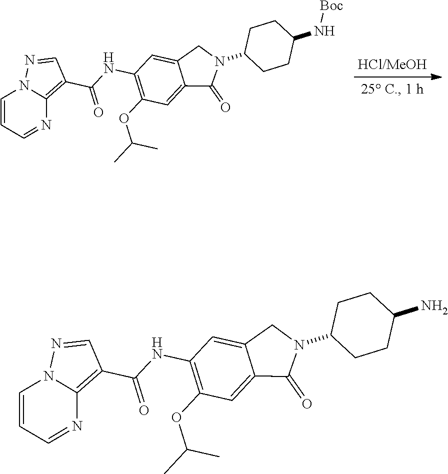 [SMILES: CC(C)Oc1cc2c(cc1NC(=O)c1cnn3cccnc13)CN([C@H]1CC[C@H](NC(=O)OC(C)(C)C)CC1)C2=O ; CC(C)Oc1cc2c(cc1NC(=O)c1cnn3cccnc13)CN([C@H]1CC[C@H](N)CC1)C2=O]

To a flask charged with tert-butyl N-[4-[6-isopropoxy-1-oxo-5-(pyrazolo[1,5-a]pyrimidine-3-carbonylamino)isoindolin-2-yl]cyclohexyl]carbamate (570 mg, 1.04 mmol, 1 eq) was added a solution of hydrochloric acid in methanol (5 mL). The mixture was stirred at 25° C. for 1 h. The aqueous phase was neutralized with ammonium hydroxide such that the pH=7, then the mixture was concentrated under reduced pressure. The crude product, N-[2-(4-aminocyclohexyl)-6-isopropoxy-1-oxo-isoindolin-5-yl]pyrazolo[1,5-a]pyrimidine-3-carboxamide (460 mg, 1.03 mmol, 98% yield) was obtained as a yellow solid and used in the next step without further purification. MS (ESI) m/z: 449.3[M+H]⁺.

### Step 6:

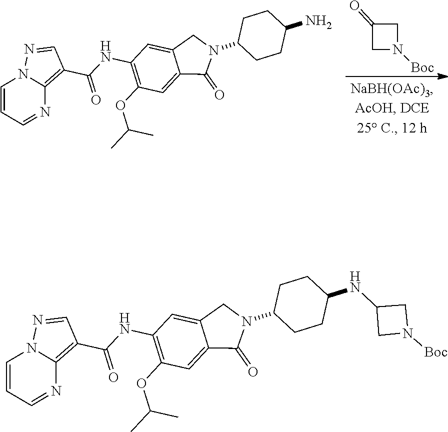 [SMILES: CC(C)Oc1cc2c(cc1NC(=O)c1cnn3cccnc13)CN([C@H]1CC[C@H](N)CC1)C2=O ; CC(C)(C)OC(=O)N1CC(=O)C1 ; CC(C)Oc1cc2c(cc1NC(=O)c1cnn3cccnc13)CN([C@H]1CC[C@H](NC3CN(C(=O)OC(C)(C)C)C3)CC1)C2=O]

To a solution of N-[2-(4-aminocyclohexyl)-6-isopropoxy-1-oxo-isoindolin-5-yl]pyrazolo[1,5-a]pyrimidine-3-carboxamide (460 mg, 1.03 mmol, 1 eq) and tert-butyl 3-oxoazetidine-1-carboxylate (526.73 mg, 3.08 mmol, 3 eq) in dichloroethane (5 mL) was added acetic acid (61.59 mg, 1.03 mmol, 58.66 uL, 1 eq). The reaction mixture was stirred at 25° C. for 0.5 h, after which sodium triacetoxyborohydride (652.10 mg, 3.08 mmol, 3 eq) was added. The mixture was stirred at 25° C. for 11.5 h. The reaction mixture was quenched by the addition of water (50 mL) at 0° C., the aqueous phase was acidified with an aqueous solution of sodium bicarbonate until the pH=7. The mixture was then diluted with water (60 mL) and extracted with ethyl acetate (50 mL×3). The combined organic layers were washed with brine (50 mL×3), dried over anhydrous sodium sulfate, filtered, and concentrated under reduced pressure to give a residue. The residue was purified by column chromatography (silicon dioxide, eluted with dichloromethane/methanol=1/0 to 20/1) to afford tert-butyl 3-[[4-[6-isopropoxy-1-oxo-5-(pyrazolo[1,5-a]pyrimidine-3-carbonylamino)isoindolin-2-yl]cyclohexyl]amino]azetidine-1-carboxylate (510 mg, 844.8 μmol, 82% yield) as a yellow solid. MS (ESI) m/z: 604.3[M+H]⁺.

### Step 7:

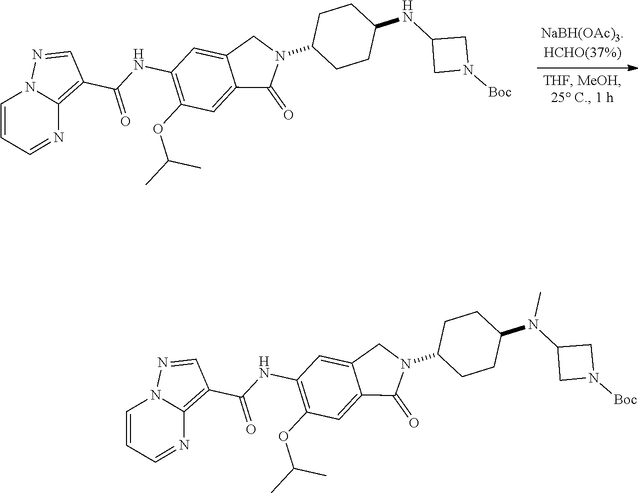 [SMILES: CC(C)Oc1cc2c(cc1NC(=O)c1cnn3cccnc13)CN([C@H]1CC[C@H](NC3CN(C(=O)OC(C)(C)C)C3)CC1)C2=O ; CC(C)Oc1cc2c(cc1NC(=O)c1cnn3cccnc13)CN([C@H]1CC[C@H](N(C)C3CN(C(=O)OC(C)(C)C)C3)CC1)C2=O]

To a solution of tert-butyl 3-[[4-[6-isopropoxy-1-oxo-5-(pyrazolo[1,5-a]pyrimidine-3-carbonylamino)isoindolin-2-yl]cyclohexyl]amino]azetidine-1-carboxylate (660 mg, 1.09 mmol, 1 eq) and formaldehyde (1.09 g, 13.43 mmol, 1 mL, 12.29 eq) in methanol (5 mL) and tetrahydrofuran (5 mL) was added sodium borohydride acetate (463.4 mg, 2.19 mmol, 2 eq). The mixture was stirred at 25° C. for 1 h. The reaction mixture was concentrated under reduced pressure. The residue was purified by column chromatography (silicon dioxide, dichloromethane/methanol=1/0 to 20/1) to afford tert-butyl 3-[[4-[6-isopropoxy-1-oxo-5-(pyrazolo[1,5-a]pyrimidine-3-carbonylamino)isoindolin-2-yl]cyclohexyl]-methyl-amino]azetidine-1-carboxylate (670 mg, 1.08 mmol, 99% yield) as a yellow solid. MS (ESI) m/z: 618.4[M+H]⁺.

### Step 8:

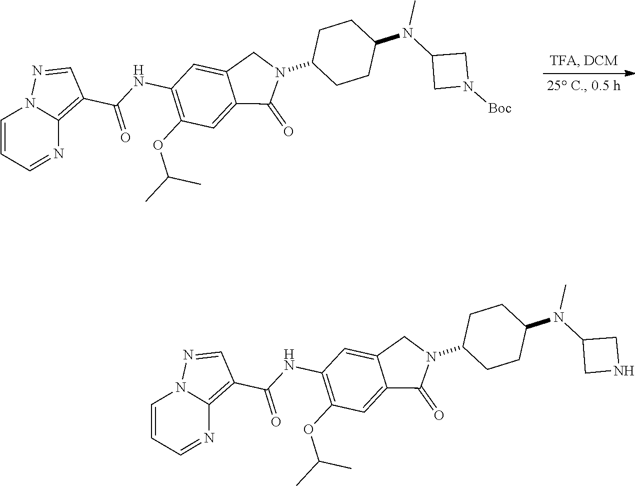 [SMILES: CC(C)Oc1cc2c(cc1NC(=O)c1cnn3cccnc13)CN([C@H]1CC[C@H](N(C)C3CN(C(=O)OC(C)(C)C)C3)CC1)C2=O ; CC(C)Oc1cc2c(cc1NC(=O)c1cnn3cccnc13)CN([C@H]1CC[C@H](N(C)C3CNC3)CC1)C2=O]

To a solution of tert-butyl 3-[[4-[6-isopropoxy-1-oxo-5-(pyrazolo[1,5-a]pyrimidine-3-carbonylamino)isoindolin-2-yl]cyclohexyl]-methyl-amino]azetidine-1-carboxylate (670 mg, 1.08 mmol, 1 eq) in dichloromethane (5 mL) was added trifluoroacetic acid (5 mL). The mixture was stirred at 25° C. for 0.5 h. The solution was neutralized with ammonium hydroxide until the pH=7, then the mixture was concentrated under reduced pressure. The crude product, N-[2-[4-[azetidin-3-yl (methyl) amino]cyclohexyl]-6-isopropoxy-1-oxo-isoindolin-5-yl]pyrazolo[1,5-a]pyrimidine-3-carboxamide (550 mg, 1.06 mmol, 97% yield) was obtained as a yellow solid and was used in the next step without further purification. MS (ESI) m/z: 518.3[M+H]⁺.

### Step 9:

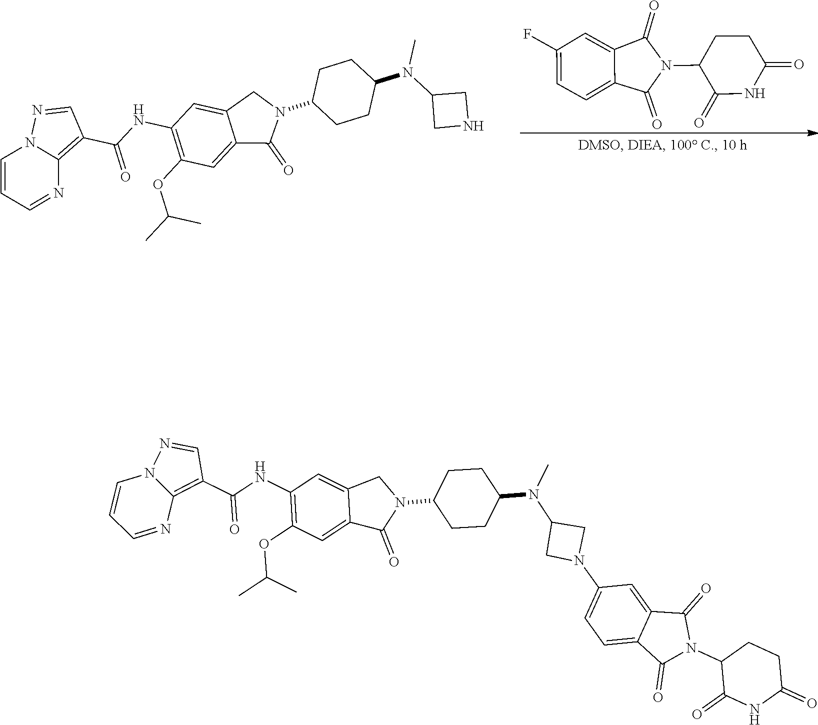 [SMILES: CC(C)Oc1cc2c(cc1NC(=O)c1cnn3cccnc13)CN([C@H]1CC[C@H](N(C)C3CNC3)CC1)C2=O ; O=C1CCC(N2C(=O)c3ccc(F)cc3C2=O)C(=O)N1 ; CC(C)Oc1cc2c(cc1NC(=O)c1cnn3cccnc13)CN([C@H]1CC[C@H](N(C)C3CN(c4ccc5c(c4)C(=O)N(C4CCC(=O)NC4=O)C5=O)C3)CC1)C2=O]

To a solution of N-[2-[4-[azetidin-3-yl(methyl)amino]cyclohexyl]-6-isopropoxy-1-oxo-isoindolin-5-yl]pyrazolo[1,5-a]pyrimidine-3-carboxamide (100 mg, 193.2 μmol, 1 eq) in dimethyl sulfoxide (2 mL) was added diisopropylethylamine (124.8 mg, 965.96 μmol, 168.25 μL, 5 eq) and 2-(2,6-dioxo-3-piperidyl)-5-fluoro-isoindoline-1,3-dione (70 mg, 253.42 μmol, 1.31 eq). The mixture was stirred at 100° C. for 10 h. The reaction mixture was partitioned between water (50 mL) and dichloromethane (50 mL×3). The combined organic phases were washed with brine (50 mL×3), dried over anhydrous sodium sulfate, filtered, and concentrated under reduced pressure to give a residue. The residue was purified by preparative thin layer chromatography (silicon dioxide, dichloromethane:methanol=10:1) to afford N-[2-[4-[[1-[2-(2,6-dioxo-3-piperidyl)-1,3-dioxo-isoindolin-5-yl]azetidin-3-yl]-methyl-amino]cyclohexyl]-6-isopropoxy-1-oxo-isoindolin-5-yl]pyrazolo[1,5-a]pyrimidine-3-carboxamide (51.1 mg, 62.7 μmol, 32% yield, 95% purity) as a yellow solid. MS (ESI) m/z: 774.3[M+H]⁺. ¹H NMR (400 MHz, DMSO-d₆): δ ppm 11.07 (s, 1H) 10.74 (s, 1H) 9.38 (br d, J=6.8 Hz, 1H) 8.88 (br d, J=2.8 Hz, 1H) 8.73 (br d, J=6.4 Hz, 2H) 7.63 (br d, J=8.2 Hz, 1H) 7.30-7.37 (m, 2H) 6.77 (s, 1H) 6.64 (br d, J=7.8 Hz, 1H) 5.06 (br dd, J=12.8, 5.2 Hz, 1H) 4.85-4.95 (m, 1H) 4.38 (br s, 2H) 4.05-4.15 (m, 2H) 3.94-4.04 (m, 1H) 3.86 (br s, 3H) 2.82-2.95 (m, 1H) 2.54-2.62 (m, 3H) 2.20 (s, 3H) 1.96-2.07 (m, 1H) 1.69-1.84 (m, 4H) 1.56-1.68 (m, 2H) 1.46-1.54 (m, 2H) 1.44 (br d, J=5.6 Hz, 6H).

TABLE 7
The following compounds may be prepared in an analogous fashion as described for the
synthesis of Example 313.

| | | ¹H NMR |
|---|---|---|
| | | All spectra acquired at 400 MHz |
| Example | MS | unless otherwise indicated |
| Example 313 | See Above | |
| 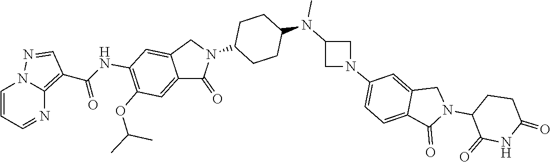 | 760.3 | CDCl3 δ ppm 10.71 (s, 1 H) 8.83-8.87 (m, 2 H) 8.79 (s, 1 H) 8.73 (dd, J = 4.0, 1.6 Hz, 1 H) 8.12-8.16 (m, 1 H) 7.70 (d, J = 8.4 Hz, 1 H) 7.39 (s, 1 H) 7.08 (dd, J = 6.8, 4.0 Hz, 1 H) 6.49 (dd, J = 8.4, 2.0 Hz, 1 H) 6.39-6.42 (m, 1 H) 5.19 (dd, J = 13.2, 5.2 Hz, 1 H) 4.76-4.86 (m, 1 H) 4.36-4.43 (m, 1 H) 4.30-4.34 (m, 2 H) 4.18-4.28 (m, 2 H) 4.03-4.09 (m, 2 H) 3.81-3.86 (m, 2 H) 2.76- |
| Example 314 | | 2.94 (m, 2 H) 2.50-2.60 (m, 1 H) |
| | | 2.29-2.35 (m, 1 H) 2.27 (s, 3 H) 2.16- |
| | | 2.24 (m, 1 H) 1.96-2.04 (m, 2 H) 1.82- |
| | | 1.90 (m, 2 H) 1.57-1.68 (m, 5 H) 1.52 |
| | | (d, J = 6.0 Hz, 6 H) |
| 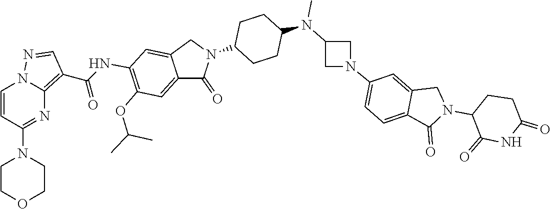 | 859.2 | DMSO-d₆ δ = 11.08 (s, 1 H), 9.69 (s, 1 H), 8.86 (d, J = 8.0 Hz, 1 H), 8.35 (s, 1 H), 8.32 (s, 1 H), 7.65 (d, J = 8.4 Hz, 1 H), 7.32 (s, 1 H), 6.95 (d, J = 8.0 Hz, 1 H), 6.80 (d, J = 1.83 Hz, 1 H), 6.62- 6.71 (m, 1 H), 5.06 (dd, J = 12.8, 4.8 Hz, 1 H), 4.62-4.72 (m, 1 H), 4.37 (s, 2 H), 4.12 (s, 2 H), 3.99 (s, 1 H), 3.88 (s, 3 H), 3.82 (s, 4 H), 3.74 (s, 4 H), 2.79- 2.95 (m, 1 H), 2.54 (s, 3 H), 2.21 (s, 3 H), 2.00 (d, J = 13.2 Hz, 1 H), 1.76 (d, J = 14.4 Hz, 4 H), 1.58-1.68 (m, 2 H), 1.44-1.55 (m, 2 H), 1.27 (d, J = 6.0 Hz, 6H). |
| Example 315 | | |
| 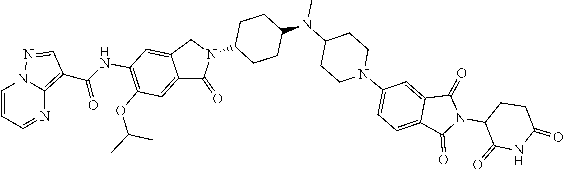 | 802.4 | DMSO-d₆ δ = 11.08 (s, 1H), 10.73 (s, 1H), 9.39 (dd, J = 1.6, 7.2 Hz, 1H), 8.88 (dd, J = 1.6, 4.4 Hz, 1H), 8.72 (s, 2H), 7.66 (d, J = 8.8 Hz, 1H), 7.37-7.30 (m, 3H), 7.24 (dd, J = 2.0, 8.8 Hz, 1H), 5.07 (dd, J = 5.6, 12.8 Hz, 1H), 4.95-4.77 (m, 1H), 4.37 (s, 2H), 4.14-3.92 (m, 3H), 3.06-2.80 (m, 4H), 2.66-2.53 (m, 3H), 2.25-2.18 (m, 2H), 2.07-1.98 (m, 1H), 1.84-1.74 (m, 5H), 1.71-1.59 (m, 2H), 1.56-1.48 (m,3H), 1.43 |
| Example 316 | | (d, J = 6.0 Hz, 6H), 1.28-1.20 |
| | | (m, 1H), 0.89-0.78 (m, 2H) |
| 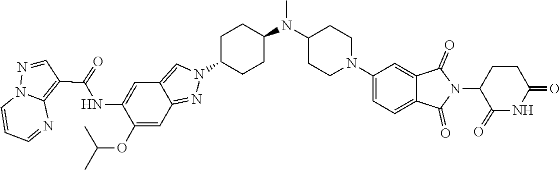 [SMILES: CC(C)Oc1cc2nn([C@H]3CC[C@H](N(C)C4CCN(c5ccc6c(c5)C(=O)N(C5CCC(=O)NC5=O)C6=O)CC4)CC3)cc2cc1NC(=O)c1cnn2cccnc12] | 787.1 | DMSO-d₆ δ = 11.37 (s, 1H), 10.88 (s, 1H), 9.67 (dd, J = 1.6, 6.8 Hz, 1H), 9.16 (dd, J = 1.6,4.4 Hz, 1H), 9.01 (d, J = 13.4 Hz, 2H), 8.54 (s, 1H), 7.95 (d, J = 8.4 Hz, 1H), 7.65-7.60 (m, 2H), 7.54 (dd, J = 2.0, 8.4 Hz, 1H), 7.41 (s, 1H), 5.36 (dd, J = 5.6, 12.4 Hz, 1H), 5.14 (td, J = 6.0, 12.0 Hz, 1H), 4.68-4.58 (m, 1H), 4.37 (br d, J = 12.8 Hz, 2H), 3.30(br t, J = 11.6 Hz, 2H), 3.20-3.12 (m, 1H), 3.12-3.05 (m, 1H), 3.05- |
| Example 317 | | 2.99 (m, 1H), 2.98-2.93 (m, 1H), 2.90 |
| | | (br d, J = 2.8 Hz, 1H), 2.86 (br d, J = |
| | | 3.6 Hz, 1H), 2.83 (s, 1H), 2.45 (br |
| | | d, J = 12.0 Hz, 2H), 2.32 (br d, J = 5.2 |
| | | Hz, 1H), 2.28 (br d, J = 3.6 Hz, 1H), |
| | | 2.25 (br s, 1H), 2.22 (br s, 1H),2.17(br |
| | | d, J = 3.2 Hz, 1H), 2.15-2.10 (m, 2H), |
| | | 2.09 (br s, 1H), 1.93-1.79 (m, 4H), 1.76 |
| | | (d, J = 6.0 Hz, 6H). |
| 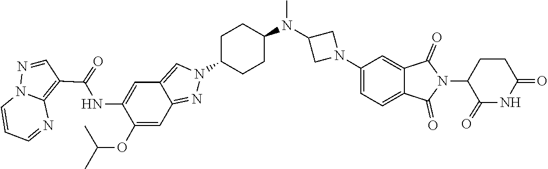 [SMILES: CC(C)Oc1cc2nn([C@H]3CC[C@H](N(C)C4CN(c5ccc6c(c5)C(=O)N(C5CCC(=O)NC5=O)C6=O)C4)CC3)cc2cc1NC(=O)c1cnn2cccnc12] | 759.3 | DMSO-d₆ δ = 11.07 (s, 1 H), 10.59 (s, 1 H), 9.37 (d, J = 6.8 Hz, 1 H), 8.87 (d, J = 2.4 Hz, 1 H), 8.75 (s, 1 H), 8.70 (s, 1 H), 8.26 (s, 1 H), 7.65 (d, J = 8.0 Hz, 1 H), 7.33 (dd, J = 7.2, 4.4 Hz, 1 H), 7.13 (s, 1H), 6.79 (s, 1H), 6.66 (d,J = 8.4 Hz, 1H), 5.06 (dd, J = 12.4, 5.2 Hz, 1 H), 4.79-4.91 (m, 1 H), 4.35 (s, 1H), 4.13 (s, 2 H), 3.89 (s, 3 H), 2.80-2.95 (m, 2 H), 2.23 (s, 3 H), 2.10-2.20 (m, 3 H), 1.86-2.05 (m, 4 H), 1.80 (d, J = |
| Example 318 | | 12.8 Hz, 2H), 1.56 (d, J = 12.8 |
| | | Hz, 2 H), 1.47 (d, J = 6.0 Hz, 6 H) |
| 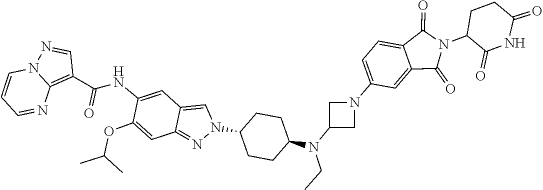 [SMILES: CCN(C1CN(c2ccc3c(c2)C(=O)N(C2CCC(=O)NC2=O)C3=O)C1)[C@H]1CC[C@H](n2cc3cc(NC(=O)c4cnn5cccnc45)c(OC(C)C)cc3n2)CC1] | 773.4 | DMSO-d₆ δ = 11.08 (s, 1 H), 10.59 (s, 1 H), 9.43-9.31 (m, 1 H), 8.90-8.82 (m, 1 H), 8.79-8.64 (m, 2 H), 8.26 (s, 1 H), 7.64 (d, J = 8.0 Hz, 1 H), 7.33 (dd, J = 4.4, 6.8 Hz, 1 H), 7.13 (s, 1H), 6.79 (s, 1 H), 6.71-6.58 (m, 1 H), 5.06 (dd, J = 5.2, 12.4 Hz, 1 H), 4.85 (m, 1 H), 4.44- 4.27 (m, 1 H), 4.22-4.00 (m, 3 H), 3.93-3.83 (m, 2H), 2.97-2.78 (m, 1 H), 2.76-2.62 (m, 3 H), 2.58-2.53 (m, 2H), 2.21-2.10 (m, 2H), 2.04-1.90 (m, 3 H), 1.87-1.77 (m, 2H), 1.64-1.50 (m, 2 H), |
| Example 319 | | 1.47 (d, J = 6.0 Hz, 6H), 1.04 (m, 3 H) |
| 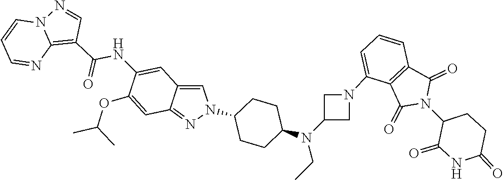 [SMILES: CCN(C1CN(c2cccc3c2C(=O)N(C2CCC(=O)NC2=O)C3=O)C1)[C@H]1CC[C@H](n2cc3cc(NC(=O)c4cnn5cccnc45)c(OC(C)C)cc3n2)CC1] | 773.6 | DMSO-d₆ δ = 11.09 (s, 1 H), 10.59 (s, 1 H), 9.38 (dd, J = 1.6, 7.2 Hz, 1 H), 8.86 (dd, J = 1.6, 4.4 Hz, 1 H), 8.74 (s, 1 H), 8.70 (s, 1 H), 8.28-8.25 (m, 1 H), 7.57 (dd, J = 7.2, 8.4 Hz, 1 H), 7.36-7.30 (m, 1 H), 7.15-7.09 (m, 2 H), 6.81 (d, J = 8.4 Hz, 1H), 5.06 (dd, J = 5.2, 12.8 Hz, 1 H), 4.85 (m, 1 H), 4.41-4.27 (m, 3 H), 3.97 (m, 3 H), 2.92-2.82 (m, 1 H), 2.70-2.56 (m, 5 H), 2.20-2.10 (m, 2H), 2.06-1.89 (m, 3 H), 1.81 (m, 2 H), 1.47 (m, 8 H), 1.03 (t ,J =6.8 Hz, 3 |
| Example 320 | | H), 0.13-−0.11 (m, 1H) |
| 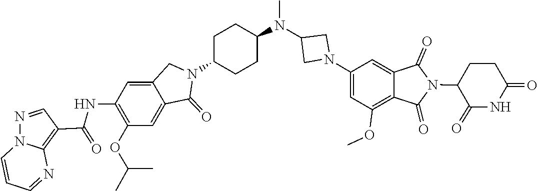 [SMILES: COc1cc(N2CC(N(C)[C@H]3CC[C@H](N4Cc5cc(NC(=O)c6cnn7cccnc67)c(OC(C)C)cc5C4=O)CC3)C2)cc2c1C(=O)N(C1CCC(=O)NC1=O)C2=O] | 804.5 | DMSO-d₆ δ = 11.05 (s, 1 H), 10.76 (s, 1 H),9.40 (dd, J = 1.6, 6.8 Hz, 1 H), 8.89 (dd, J = 1.6, 4.0 Hz, 1H),8.74 (d, J = 2.8 Hz, 2 H), 7.39-7.31 (m, 2 H), 6.42 (s, 1 H), 6.14 (s, 1 H), 5.03-4.95 (m, 1 H), 4.90 (m, 1 H), 4.39 (s, 2 H), 4.18-4.09 (m, 2H), 4.03-3.95 (m, 1 H), 3.93-3.84 (m, 6 H), 2.94-2.81 (m, 1 H), 2.57 (m, 3 H), 2.21 (s, 3 H), 2.00-1.93 (m, 1 H), 1.83-1.72 (m, 4 H), 1.68-1.58 (m, 2 H), 1.55-1.47 (m, 2 H), 1.44 (d, J = 6.0 Hz, 6H) |
| Example 321 | | |
| 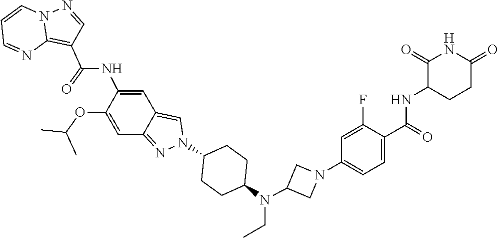 [SMILES: CCN(C1CN(c2ccc(C(=O)NC3CCC(=O)NC3=O)c(F)c2)C1)[C@H]1CC[C@H](n2cc3cc(NC(=O)c4cnn5cccnc45)c(OC(C)C)cc3n2)CC1] | 765.3 | DMSO-d₆ δ = 10.85 (s, 1 H), 10.59 (s, 1 H), 9.38 (dd, J = 1.2, 7.2 Hz, 1 H), 8.87 (dd, J = 1.2, 4.0 Hz, 1 H), 8.74 (s, 1 H), 8.71 (s, 1H), 8.26 (s, 1 H), 7.99 (t, J = 7.2 Hz, 1 H),7.62 (t, J = 8.4 Hz, 1 H), 7.33 (dd, J = 4.4, 7.2 Hz, 1 H), 7.12 (s, 1 H), 6.34-6.19 (m, 2 H), 4.85 (m, 1 H), 4.77-4.67 (m, 1 H), 4.41-4.29 (m, 1 H), 4.03 (m, 3 H), 3.74 (m, 2 H), 2.84-2.73 (m, 1 H), 2.71-2.61 (m, 3 H), 2.21-2.09 (m, 3 H), 2.08-1.86 (m, 4 H), 1.85-1.77 (m, 2 H), 1.47 (d, J = 6.0 Hz, 8H), 1.03 (t, J = 6.8 Hz, 3 H) |
| Example 322 | | |
| 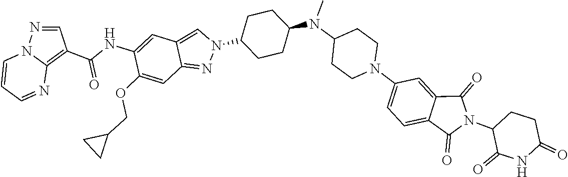 | 799.2 | DMSO-d₆ δ = 11.08 (s, 1 H), 10.64 (s, 1 H), 9.37 (dd, J = 7.2, 1.6 Hz, 1 H), 8.87 (dd, J = 4.0, 1.6 Hz, 1 H), 8.72 (d, J = 11.2 Hz, 2 H), 8.25 (s, 1 H), 7.67 (d, J = 8.4 Hz, 1 H), 7.21-7.40 (m, 3 H), 7.02 (s, 1 H), 5.07 (dd, J = 12.8, 5.6 Hz, 1H), 4.36 (s, 1H), 4.09 (s, 1H), 4.02 (d, J = 6.8 Hz, 2 H), 3.58-3.61 (m, 1 H), 2.96-3.05 (m, 2 H), 2.77-2.93 (m, 2 H), 2.53-2.69 (m, 3 H), 2.18 (s, 4 H), 1.82-2.06 (m, 6 H), 1.76 (1, J = 3.2 |
| Example 323 | | Hz, 1 H), 1.45-1.66 (m, 4 H), 1.35 (s, 1 |
| | | H), 1.23 (s, 1 H), 0.65-0.75 (m, 2 H), |
| | | 0.45 (d, J = 4.8 Hz, 2H). |
| 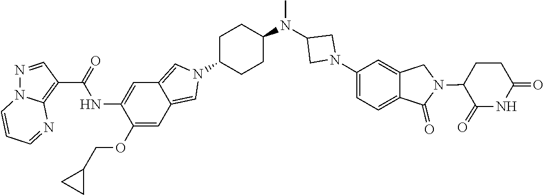 [SMILES: CN(C1CN(c2ccc3c(c2)CN(C2CCC(=O)NC2=O)C3=O)C1)[C@H]1CC[C@H](n2cc3cc(NC(=O)c4cnn5cccnc45)c(OCC4CC4)cc3c2)CC1] | 757.3 | DMSO-d₆ δ = 10.95 (s, 1 H) 10.65 (s, 1 H) 9.37 (dd, J = 6.8, 1.6 Hz, 1 H) 8.87 (dd, J = 4.0, 1.6 Hz, 1 H) 8.74 (s, 1 H) 8.71 (s, 1 H) 8.26 (s, 1 H) 7.50 (d, J = 8.0 Hz, 1 H) 7.30-7.33 (m, 1 H) 7.03 (s, 1H) 6.54 (s, 1 H) 6.51 (dd, J = 8.4, 1.6 Hz, 1 H) 5.04 (dd, J = 13.2, 5.2 Hz, 1 H) 4.27-4.40 (m, 2 H) 4.15 -4.23 (m, 1 H) 4.00-4.07 (m, 4 H) 3.80-3.89 (m, 1 H) 3.69-3.76 (m, 2 H) 2.83-2.97 (m, 1 H) 2.54-2.64 (m, 2 H) 2.36-2.43 (m, 1 H) 2.20 (s, 3 H) 2.14-2.19 (m, 2 H) 1.89-1.99 (m, 3 H) 1.81 (br d, J = |
| Example 324 | | 10.8 Hz, 2 H) 1.47-1.61 (m, 3 H) 0.68- |
| | | 0.74 (m, 2 H) 0.42-0.48 (m, 2 H) |
| 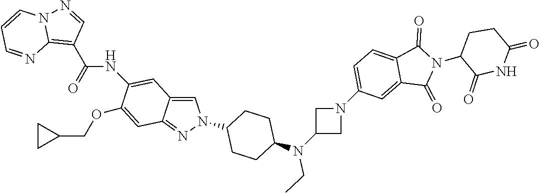 | 785.3 | DMSO-d₆ δ = 11.07 (s, 1H), 10.64 (s, 1H), 9.36 (d, J = 6.8 Hz, 1H), 8.86 (d, J = 2.8 Hz, 1H), 8.72 (d, J = 15.2 Hz, 2H), 8.25 (s, 1H), 7.63 (d, J = 8.0 Hz, 1H), 7.31 (dd, J = 4.4, 6.8 Hz, 1H), 7.09-6.96 (m, 1H), 6.78 (s, 1H), 6.65 (d, J = 7.8 Hz, 1H), 5.06 (dd, J = 5.2, 12.8 Hz, 1H), 4.38-4.30 (m, 1H), 4.20- 4.00 (m, 6H), 3.89 (d, J = 6.4 Hz, 2H), 2.89-2.83 (m, 1H), 2.68-2.63(m, 3H), 2.18-2.12 (m, 2H), 2.01-1.89 (m, 3H), 1.86-1.79 (m, 2H), 1.59-1.43 (m, 4H), 1.04 (t, J = 6.4 Hz, 3H), 0.74-0.67 |
| Example 325 | | (m, 2H), 0.50-0.42 (m, 2H) |
| 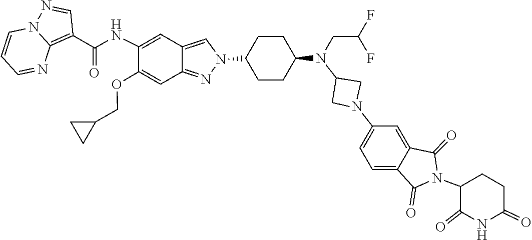 | 821.1 | DMSO-d₆ δ = 11.07 (br d, J = 1.2 Hz, 1H), 10.64 (s, 1H), 9.37 (dd, J = 1.6, 7.2 Hz, 1H), 8.87 (dd, J = 1.6, 4.0 Hz, 1H), 8.72 (d, J = 11.6 Hz, 2H), 8.26 (s, 1H), 7.65 (d, J = 8.4 Hz, 1H), 7.32 (dd, J = 4.0, 6.8 Hz, 1H), 7.03 (s, 1H), 6.80 (d, J = 1.6 Hz, 1H), 6.67 (dd, J = 1.6, 8.4 Hz, 1H), 5.06 (dd, J = 5.2, 12.8 Hz, 1H), 4.44-4.29 (m, 1H), 4.25-4.12 (m, 3H), 4.02 (d, J = 6.8 Hz, 2H), 3.90 (br d, J = 5.2 Hz, 2H), 3.14-3.01 (m, 2H), 2.95-2.82 (m, 1H), 2.81-2.74 (m, 1H), 2.70-2.64 (m, 1H), 2.63-2.58 (m, 1H), 2.54 (s, 2H), 2.37-2.29 (m, 1H), 2.24-2.09 (m, 2H), 1.95-1.90 (m, 1H), 1.85 |
| Example 326 | | (br dd, J = 3.2, 14.8 Hz, 2H), 1.61-1.46 |
| | | (m, 3H), 0.71 (br d, J = 8.0 Hz, 2H), |
| | | 0.46 (br d, J = 5.2 Hz, 2H) |
| 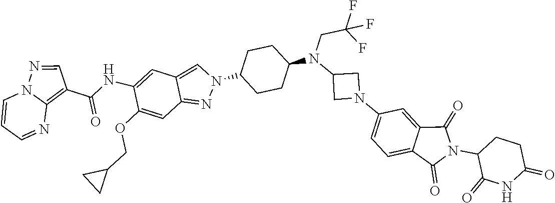 | 839.2 | DMSO-d₆ δ = 11.07 (s, 1H), 10.64 (s, 1H), 9.35 (dd, J = 1.6, 7.2 Hz, 1H), 8.85 (dd, J = 1.6, 4.0 Hz, 1H), 8.74 (s, 1H), 8.70 (s, 1H), 8.26 (s, 1H), 7.63 (d, J = 8.0 Hz, 1H), 7.34-7.25 (m, 1H), 7.03 (s, 1H), 6.79 (d, J = 1.6 Hz, 1H), 6.66 (dd, J = 1.6, 8.4 Hz, 1H), 5.06 (dd, J = 5.2, 12.8 Hz, 1H), 4.38-4.29 (m, 1H), 4.22-4.14 (m, 3H), 4.02 (d, J = 6.8 Hz, 2H), 3.92-3.82 (m, 2H), 3.57-3.48 (m, 2H), 2.94-2.77 (m, 2H), 2.62-2.55 (m, 2H), 2.21-2.09 (m, 2H), 2.03-1.81 (m, 5H), 1.60-1.45 (m, 3H), 0.76-0.67 |
| Example 327 | | (m, 2H), 0.49-0.41 (m, 2H) |
| 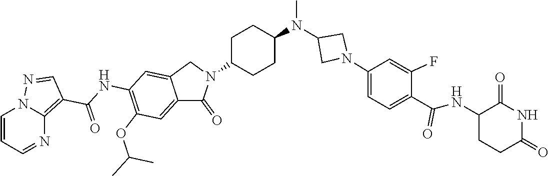 | 766.2 | DMSO-d₆ δ = 10.85 (s, 1H), 10.76 (s, 1H), 9.41 (d, J = 7.2 Hz, 1H), 8.90 (d, J = 2.8 Hz, 1H), 8.74 (s, 2H), 8.05-7.94 (m, 1H), 7.62 (1, J = 8.8 Hz, 1H), 7.42-7.31 (m, 2H), 6.37-6.18 (m, 2H), 4.97-4.85 (m, 1H), 4.77-4.67 (m, 1H), 4.43-4.37 (m, 2H), 4.05-3.97 (m, 3H), 3.87-3.70 (m, 4H), 2.83-2.72 (m, 2H), 2.22-2.14 (m, 3H), 2.05-1.98 (m, 1H), 1.85-1.71 (m, 4H), 1.69-1.60 (m, 2H), 1.44 (d, J = 6.0 Hz, 6H), 1.30-1.22 (m, 3H) |
| Example 328 | | |
| 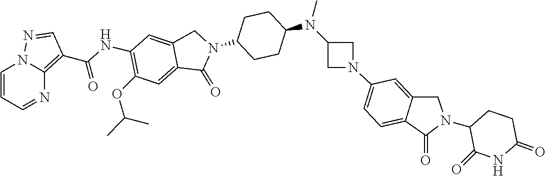 | 727.2 | DMSO-d₆ δ = 10.94 (s, 1 H), 10.75 (s, 1 H),9.40 (dd, J = 7.2, 1.6 Hz, 1 H), 8.89 (dd, J = 4.0, 1.6 Hz, 1 H), 8.73 (s, 2 H), 7.49 (d, J = 8.4 Hz, 1H), 7.30-7.38 (m, 2 H), 6.46-6.57 (m, 2 H), 5.04 (dd, J = 13.2, 5.2 Hz, 1 H), 4.90 (dt, J = 12.0, 6.0 Hz, 1H), 4.39 (s, 2 H), 4.16-4.34 (m, 2 H), 3.96-4.07 (m, 3 H), 3.79-3.87 (m, 1 H), 3.71 (d, J = 6.4 Hz, 2 H), 2.85-2.97 (m, 1 H), 2.58 (d, J = 17.2 Hz, 1 H), 2.31-2.40 (m, 1 H), 2.19 (s, 3 H), 1.92-2.00 (m, 1 H), 1.78 |
| Example 329 | | (t ,J = 13.6 Hz, 4 H), 1.58-1.67 (m, 2 |
| | | H), 1.47-1.55 (m, 2 H), 1.44 (d, J = |
| | | 6.0 Hz, 6H), 1.22-1.36 (m, 1 H). |
| 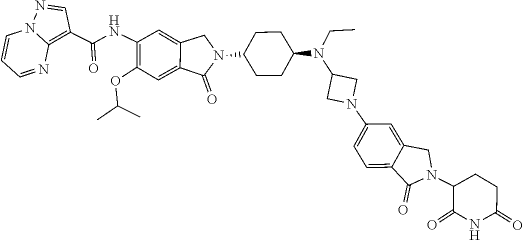 | 788.3 | DMSO-d₆ δ = 11.08 (s, 1H), 10.75 (s, 1H), 9.40 (dd, J = 1.6, 7.2 Hz, 1H), 8.89 (dd, J = 1.6, 4.0 Hz, 1H), 8.74 (s, 2H), 7.64 (d, J = 8.4 Hz, 1H), 7.40-7.28 (m, 2H), 6.80 (d, J = 2.0 Hz, 1H), 6.69- 6.60 (m, 1H), 5.11-5.01 (m, 1H), 4.96- 4.85 (m, 1H), 4.39 (s, 2H), 4.18-3.99 (m, 4H), 3.91-3.84 (m, 2H), 2.95-2.82 (m, 1H), 2.64-2.59 (m, 2H), 2.55-2.53 (m, 3H), 2.07-1.97 (m, 1H), 1.81-1.75 (m, 3H), 1.69-1.63 (m, 1H), 1.49-1.40 (m, 8H), 1.24-1.21 (m, 1H), 1.03 (t, J = 6.8 Hz, 3H), 0.88-0.78 (m, 1H) |
| Example 330 | | |
| 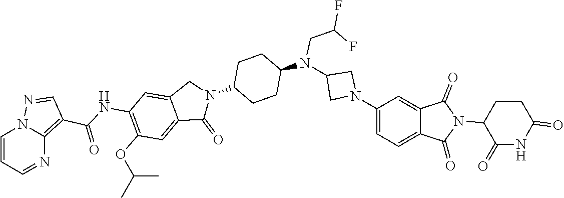 | 824.2 | DMSO-d₆ δ = 11.07 (s, 1 H), 10.75 (s, 1 H), 9.35-9.44 (m, 1 H), 8.89 (dd, J = 4.0, 1.6 Hz, 1H), 8.73 (d, J = 1.6 Hz, 2 H), 7.65 (d, J = 8.4 Hz, 1H), 7.30-7.42 (m, 2 H), 6.79 (s, 1 H), 6.66 (dd, J = 8.4, 2.0 Hz, 1 H), 5.83-6.19 (m, 1 H), 5.06 (dd, J = 12.8, 5.2 Hz, 1H), 4.90 (dt, J = 12.0, 6.0 Hz, 1H), 4.39 (s, 2 H), 4.16 (s, 3 H), 4.03 (t ,J = 11.6 Hz, 1 H), 3.87 (d, J = 4.0 Hz, 2 H), 3.00-3.12 (m, 2 H), 2.81-2.94 (m, 1 H), 2.53-2.68 (m, 3 H), 1.97-2.06 (m, 1H), 1.79 (d, J = 9.6 Hz, 4 H), 1.58-1.70 (m, 2 H), |
| Example 331 | | 1.46-1.55 (m, 2 H), 1.44 |
| | | (d, J = 6.0 Hz, 6 H). |
| 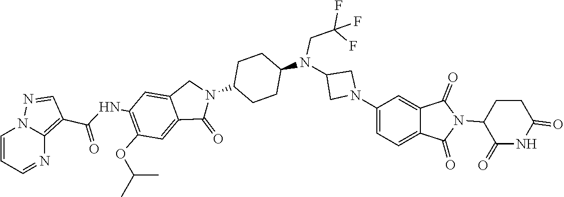 | 842.3 | DMSO-d₆ δ = 11.09 (s, 1H), 10.76 (s, 1H), 9.41 (dd, J = 1.2, 7.0 Hz, 1H), 8.89 (dd, J = 1.6, 4.2 Hz, 1H), 8.74 (s, 2H), 7.65 (d, J = 8.4 Hz, 1H), 7.40-7.31 (m, 2H), 6.81 (d, J = 1.6 Hz, 1H), 6.68 (dd, J = 1.8, 8.4 Hz, 1H), 5.06 (dd, J = 5.4, 12.8 Hz, 1H), 4.90 (td, J = 6.0, 12.2 Hz, 1H), 4.39 (s, 2H), 4.28-4.14 (m, 3H), 4.08-3.98 (m, 1H), 3.91-3.82 (m, 2H), 3.59-3.46 (m, 2H), 2.94-2.81 (m, 1H), 2.72-2.64 (m, 1H), 2.58-2.52 (m, 2H), 2.07-1.96 (m, 1H), 1.79 (br d, J = 9.4 Hz, 4H), 1.71-1.48 (m, 4H), |
| Example 332 | | 1.44 (d, J = 6.0 Hz, 6H) |
| 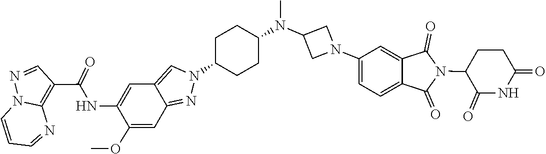 [SMILES: COc1cc2nn([C@H]3CC[C@@H](N(C)C4CN(c5ccc6c(c5)C(=O)N(C5CCC(=O)NC5=O)C6=O)C4)CC3)cc2cc1NC(=O)c1cnn2cccnc12] | 731.2 | CDCl3 δ = 10.47 (s, 1H), 8.86-8.82 (m, 2H), 8.82-8.82 (m, 1H), 8.80 (s, 1H), 8.76 (dd, J = 1.6, 4.4 Hz, 1H), 7.97 (s, 2H), 7.65 (d, J = 8.4 Hz, 1H), 7.14-7.03 (m, 2H), 6.80 (d, J = 2.1 Hz, 1H), 6.54 (dd, J = 2.1, 8.4 Hz, 1H), 4.93 (dd, J = 5.6, 12.4 Hz, 1H), 4.58 (br d, J = 3.6 Hz, 1H), 4.12-4.07 (m, 2H), 4.06-4.05 (m, 1H), 4.06-4.05 (m, 1H), 4.06 (s, 3H), 4.06-4.05 (m, 1H), 3.99-3.90 (m, |
| Example 333 | | 3H), 2.96-2.56 (m, 6H), 2.24 (s, 3H), |
| | | 2.18-2.09 (m, 1H), 2.05-1.92 (m, 2H), |
| | | 1.80 (br d, J = 10.4 Hz, 2H), 1.70 (br s, |
| | | 2H), 1.73-1.66 (m, 1H) |
| 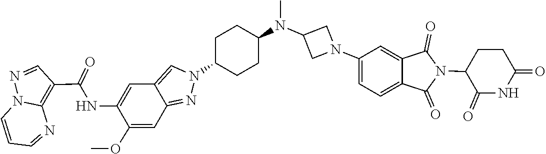 [SMILES: COc1cc2nn([C@H]3CC[C@H](N(C)C4CN(c5ccc6c(c5)C(=O)N(C5CCC(=O)NC5=O)C6=O)C4)CC3)cc2cc1NC(=O)c1cnn2cccnc12] | 731.4 | DMSO-d₆ δ = 11.08 (s, 1 H) 10.53 (s, 1 H) 9.34 (dd, J = 6.8, 1.2 Hz, 1 H) 8.91- 8.94 (m, 1 H) 8.69 (d, J = 2.0 Hz, 2 H) 8.26 (s, 1H)7.61 (d, J = 8.0 Hz, 1 H) 7.31 (dd, J = 6.8, 4.0 Hz, 1 H) 7.10 (s, 1H) 6.74 (s, 1 H) 6.59-6.63 (m, 1 H) 5.06 (br dd, J = 12.8, 5.2 Hz, 1H) 4.27-4.38 (m, 1 H) 4.05-4.13 (m, 2 H) 4.03 (s, 3 H) 3.81-3.91 (m, 3 H) 2.81-2.96 (m, 1 H) 2.52-2.62 (m, 3 H) 2.20 |
| Example 334 | | (s, 3 H) 2.13 (br d, J = 10.8 Hz, 2 H) |
| | | 1.97-2.07 (m, 1 H) 1.83-1.96 (m, 2 H) |
| | | 1.73-1.80 (m, 2 H) 1.44-1.60 (m, 2H) |
| 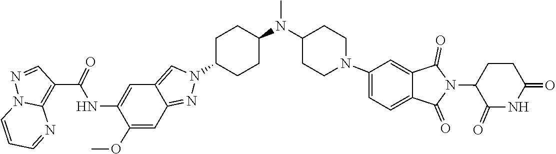 [SMILES: COc1cc2nn([C@H]3CC[C@H](N(C)C4CCN(c5ccc6c(c5)C(=O)N(C5CCC(=O)NC5=O)C6=O)CC4)CC3)cc2cc1NC(=O)c1cnn2cccnc12] | 759.2 | DMSO-d₆ δ = 10.46 (s, 1 H), 8.81-8.85 (m, 2 H), 8.79 (s, 1 H), 8.75 (dd, J = 4.0, 1.6 Hz, 1 H), 8.15 (s, 1 H), 7.85 (s, 1 H), 7.70 (d, J = 8.4 Hz, 1H), 7.30 (d, J = 2.0 Hz, 1 H), 7.03-7.10 (m, 3 H), 4.96 (dd, J = 12.4, 5.2 Hz, 1 H), 4.35 (s, 1H), 4.05 (s, 5 H), 3.02 (t, J = 12.0 Hz, 2 H), 2.74-2.94 (m, 4 H), 2.62 (s, 1H), 2.38 (d, J = 10.8 Hz, 3 H), 2.00-2.19 (m, 6 H), 1.66 (s, 4 H), 1.23- |
| Example 335 | | 1.35 (m, 2 H), 0.86-0.89 (m, 1 H). |
| 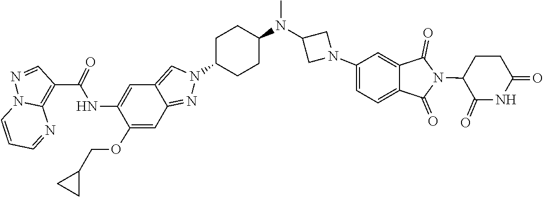 [SMILES: CN(C1CN(c2ccc3c(c2)C(=O)N(C2CCC(=O)NC2=O)C3=O)C1)[C@H]1CC[C@H](n2cc3cc(NC(=O)c4cnn5cccnc45)c(OCC4CC4)cc3n2)CC1] | 771.2 | DMSO-d₆ δ = 11.08 (s, 1 H) 10.64 (s, 1 H) 9.31-9.37 (m, 1 H) 8.82-8.87 (m, 1 H) 8.75 (s, 1 H) 8.70 (s, 1 H) 8.25 (s, 1 H) 7.59-7.64 (m, 1 H) 7.29 (dd, J = 6.8, 4.0 Hz, 1 H) 7.03 (s, 1 H) 6.75 (s, 1H) 6.62 (dd, J = 8.0, 1.6 Hz, 1 H) 5.06 (br dd, J = 12.8, 5.2 Hz, 1 H) 4.26-4.37 (m, 1 H) 4.09 (br s, 2 H) 4.02 (br d, J = 6.8 Hz, 2H) 3.87 (br d, J = 5.6 Hz, 3 H) 2.82-2.94 (m, 1 H) 2.55-2.63 (m, 3 H) 2.21 (s, 3 H) 2.13 (br d, J = 11.2 Hz, 2 H) 1.97-2.07 (m, 1 H) 1.82-1.95 (m, 2 H) 1.77 (br d, J = 10.4 Hz, 2H) 1.45- |
| Example 336 | | 1.59 (m, 3 H) 0.66-0.75 (m, 2 H) 0.41- |
| | | 0.49 (m, 2H) |
| 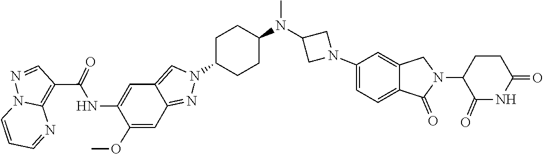 [SMILES: COc1cc2nn([C@H]3CC[C@H](N(C)C4CN(c5ccc6c(c5)CN(C5CCC(=O)NC5=O)C6=O)C4)CC3)cc2cc1NC(=O)c1cnn2cccnc12] | 717.2 | DMSO-d₆ δ = 10.90-10.98 (m, 1 H) 0.54 (s, 1 H) 9.38 (dd, J = 6.8, 1.6 Hz, 1 H) 8.96 (dd, J = 4.0, 1.6 Hz, 1H) 8.70 (s, 1 H) 8.68 (s, 1 H) 8.41 (s, 1H) 8.27 (s, 1 H) 7.50 (d, J = 8.4 Hz, 1 H)7.33-7.37 (m, 1 H) 7.10 (s, 1 H) 6.55 (s, 1H) 6.49-6.53 (m, 1 H) 5.04 (dd, J = 13.2, 5.2 Hz, 1 H) 4.28-4.41 (m, 2 H) 4.16-4.22 (m, 1 H) 4.01-4.06 (m, 5 H) 3.82-3.88 (m, 1H) 3.73 (br t, J = 5.6 Hz, 2 |
| Example 337 | | H) 2.83-2.97 (m, 1 H) 2.21 (s, 3 H) |
| | | 2.16 (br s, 2 H) 1.88-1.99 (m, 4 H) |
| | | 1.78-1.84 (m, 2 H) 1.52-1.62 (m, 2 H) |
| | | 1.23 (br s, 2 H) |
| 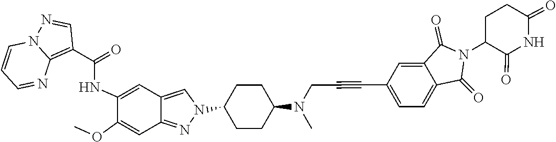 [SMILES: COc1cc2nn([C@H]3CC[C@H](N(C)CC#Cc4ccc5c(c4)C(=O)N(C4CCC(=O)NC4=O)C5=O)CC3)cc2cc1NC(=O)c1cnn2cccnc12] | 720.2 | CDCl3 δ = 10.46 (s, 1 H), 8.88-8.77 (m, 3 H), 8.75 (dd, J = 1.6, 4.0 Hz, 1 H), 8.17 (s, 1H), 7.85 (s, 1H),7.80 (d, J = 8.4 Hz, 1 H), 7.38 (d, J = 2.4 Hz, 1 H), 7.23 (dd, J = 2.0, 8.0 Hz, 1 H), 7.10-7.00 (m, 2 H), 4.97 (dd, J = 5.2, 12.0 Hz, 1 H), 4.40-4.26 (m, 1 H), 4.20 (m, 2 H), 4.05 (s, 3 H), 3.04-2.65 (m, 6 H), 2.46 (s, 3 H), 2.38 (m, 2 H), |
| Example 338 | | 2.22-1.96 (m, 5 H), 1.60-1.47 (m, 2H |
| 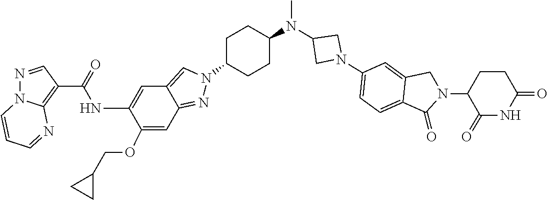 [SMILES: CN(C1CN(c2ccc3c(c2)CN(C2CCC(=O)NC2=O)C3=O)C1)[C@H]1CC[C@H](n2cc3cc(NC(=O)c4cnn5cccnc45)c(OCC4CC4)cc3n2)CC1] | 757.3 | DMSO-d₆ δ = 10.95 (s, 1 H) 10.65 (s, 1 H) 9.37 (dd, J = 6.8, 1.6 Hz, 1 H) 8.87 (dd, J = 4.0, 1.6 Hz, 1 H) 8.74 (s, 1 H)8.71 (s, 1 H) 8.26 (s, 1 H) 7.50 (d, J = 8.0 Hz, 1 H) 7.30-7.33 (m, 1 H) 7.03 (s, 1H)6.54 (s, 1 H) 6.51 (dd, J = 8.4, 1.6 Hz, 1 H) 5.04 (dd, J = 13.2, 5.2 Hz, 1 H) 4.27-4.40 (m, 2 H) 4.15 -4.23 (m, 1 H) 4.00-4.07 (m, 4 H) 3.80-3.89 (m, 1 H) 3.69-3.76 (m, 2 H) 2.83-2.97 (m, 1 H) 2.54-2.64 (m, 2 H) 2.36-2.43 (m, 1 H) 2.20 (s, 3 H) 2.14-2.19 (m, 2 H) 1.89-1.99 (m, 3 H) 1.81 (br d, J = |
| Example 339 | | 10.8 Hz, 2 H) 1.47-1.61 (m, 3 H) 0.68- |
| | | 0.74 (m, 2 H) 0.42-0.48 (m, 2 H) |
| 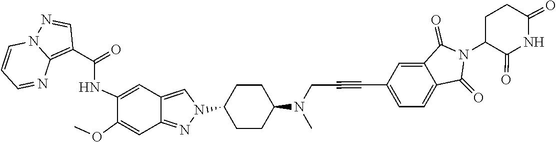 [SMILES: COc1cc2nn([C@H]3CC[C@H](N(C)CC#Cc4ccc5c(c4)C(=O)N(C4CCC(=O)NC4=O)C5=O)CC3)cc2cc1NC(=O)c1cnn2cccnc12] | 714.2 | DMSO-d₆ δ = 11.14 (s, 1 H) 10.53 (s, 1 H) 9.38 (d, J = 5.6 Hz, 1 H) 8.95 (d, J = 2.4 Hz, 1 H) 8.69 (d, J = 11.2 Hz, 2H) 8.43 (s, 1 H) 8.28 (s, 1 H) 7.93 (s, 3 H) 7.32-7.39 (m, 1 H) 7.10 (s, 1 H) 5.17 (dd, J = 12.8, 5.2 Hz, 1 H) 4.38 (s, 1H) 4.02 (s, 3 H) 3.74 (s, 2 H) 2.84-2.95 (m, 1 H) 2.55-2.71 (m, 4 H) 2.40 (s, 3 H) 2.18 (m, 2 H) 2.08 (s, 2 H) 1.88- |
| Example 340 | | 2.01 (m, 2 H) 1.41-1.57 (m, 2 H) |
| 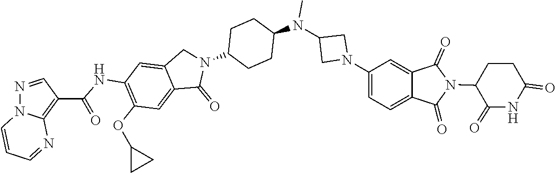 | 777.2 | DMSO-d₆ δ = 11.07 (s, 1 H), 10.56 (s, 1 H), 9.38 (dd, J = 7.2, 1.6 Hz, 1 H), 8.90 (dd, J = 4.4, 1.6 Hz, 1 H), 8.72 (s, 1 H), 8.69 (s, 1 H), 7.64 (d, J = 8.4 Hz, 1 H), 7.56 (s, 1 H), 7.32-7.38 (m, 1 H), 6.78 (d, J = 2.0 Hz, 1H), 6.65 (dd, J = 8.4, 2.0 Hz, 1H), 5.06 (dd, J = 12.8,5.2 Hz, 1H), 4.40 (s, 2 H), 4.21 (dt, J = 5.66, 2.8 Hz, 1 H), 4.11 (s, 2 H), 3.99 (t, J = 11.6 Hz, 1H),3.87 (d, J = 4.8 Hz, 3 H), 2.81-2.94 (m, 1 H), 2.51-2.62 |
| Example 341 | | (m, 3 H), 2.20 (s, 3 H), 1.97-2.07 (m, 1 |
| | | H), 1.70-1.84 (m, 4 H), 1.57-1.69 (m, 2 |
| | | H), 1.43-1.55 (m, 2 H), 0.90-0.97 (m, 2 |
| | | H), 0.83-0.90 (m, 2 H). |
| 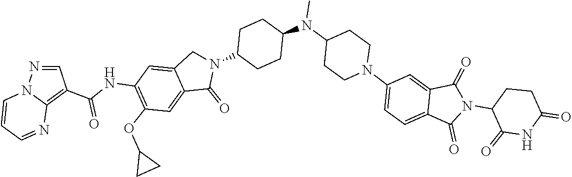 | 800.2 | DMSO-d₆ δ = 11.10 (s, 1 H), 10.57 (s, 1 H), 9.40 (dd, J = 1.6, 7.2 Hz, 1 H), 8.91 (dd, J = 1.6, 4.4 Hz, 1 H), 8.71 (d, J = 16 Hz, 2 H), 8.20 (s, 1 H), 7.67 (d, J = 8.4 Hz, 1 H), 7.56 (s, 1 H), 7.39-7.31 (m, 2 H),7.25 (dd, J = 2.0, 8.4 Hz, 1 H), 5.08 (dd, J = 5.2, 12.8 Hz, 1 H), 4.40 (s, 2 H), 4.21 (m, 1 H), 4.08 (m, 2 H), 4.02-3.96 (m, 1 H), 3.00 (m, 3 H), 2.94-2.78 (m, 3H), 2.63-2.53 (m, 2 H), 2.22 (s, 3 H), 2.06-1.98 (m, 1 H), 1.87- 1.76 (m, 6 H), 1.72-1.60 (m, 2 H), |
| Example 342 | | 1.59-1.46 (m, 4 H), 0.99-0.91 (m, 2 |
| | | H), 0.89-0.81 (m, 2 H) |
| 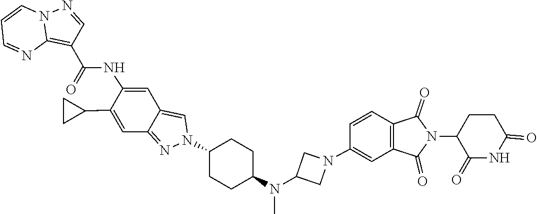 [SMILES: CN(C1CN(c2ccc3c(c2)C(=O)N(C2CCC(=O)NC2=O)C3=O)C1)[C@H]1CC[C@H](n2cc3cc(NC(=O)c4cnn5cccnc45)c(C4CC4)cc3n2)CC1] | 741.2 | DMSO-d₆ δ = 11.08 (s, 1 H), 10.34 (s, 1 H), 9.38 (dd, J = 6.8, 1.6 Hz, 1 H), 8.90 (dd, J = 4.0, 1.6 Hz, 1 H), 8.72 (s, 1 H), 8.65 (s, 1 H), 8.32 (s, 1H),7.64 (d, J = 8.4 Hz, 1 H), 7.41 (s, 1 H), 7.29-7.35 (m, 1 H), 7.29-7.35 (m, 1 H), 6.78 (s, 1 H), 6.66 (dd, J = 8.4, 2.0 Hz, 1H), 5.06 (dd, J = 12.8,5.2 Hz, 1H), 4.40 (t, J = 12.0 Hz, 1 H), 4.12 (s, 2 H), 3.89 (d, J = 4.8 Hz, 3 H), 2.82-2.94 (m, 1 H), 2.54-2.65 (m, 2 H), 2.22 (s, 3 H), 1.87-2.19 (m, 7 H), 1.75-1.85 (m, 2 H), 1.49-1.62 (m, 2 H), 1.13-1.20 (m, 2 H), 0.74-0.81 (m, 2H). |
| Example 343 | | |
| 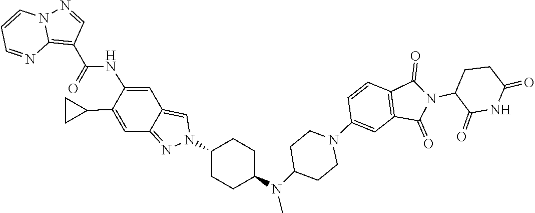 [SMILES: CN(C1CCN(c2ccc3c(c2)C(=O)N(C2CCC(=O)NC2=O)C3=O)CC1)[C@H]1CC[C@H](n2cc3cc(NC(=O)c4cnn5cccnc45)c(C4CC4)cc3n2)CC1] | 769.2 | DMSO-d₆ δ = 11.08 (s, 1 H), 10.33 (s, 1 H), 9.38 (dd, J = 7.2, 1.6 Hz, 1 H), 8.91 (dd, J = 4.0, 1.6 Hz, 1 H), 8.73 (s, 1 H), 8.63(s, 1H),8.31(s, 1 H), 8.21 (s, 1 H), 7.66 (d, J = 8.8 Hz, 1 H), 7.41 (s, 1 H), 7.30-7.36 (m, 2 H), 7.25 (d, J = 9.2 Hz, 1 H), 5.07 (dd, J = 12.8, 5.2 Hz, 1 H), 4.40 (t, J = 11.6 Hz, 1 H), 4.08 (d, J = 13.2 Hz, 2 H), 2.83-3.04 (m, 4 H), 2.74 (t, J = 10.8 Hz, 1 H), 2.53-2.63 (m, 2 H), 2.22 (s, 3 H), 1.93- 2.17 (m, 6 H), 1.79-1.91 (m, 4 H), 1.46-1.65 (m, 4 H), 1.13-1.20 (m, 2 H), 0.73-0.81 (m, 2H). |
| Example 344 | | |
| 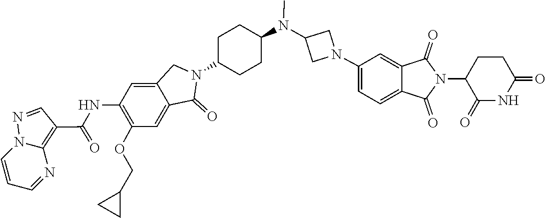 | 786.3 | DMSO-d₆ δ = 11.08 (s, 1 H), 10.81 (s, 1 H),9.38(dd, J = 1.6, 7.2 Hz, 1 H), 8.88 (dd, J = 1.6, 4.4 Hz, 1 H), 8.73 (s, 2 H), 7.63 (d, J = 8.4 Hz, 1 H), 7.39-7.29 (m, 1 H), 7.25 (s, 1 H), 6.78 (d, J = 2.0 Hz, 1 H), 6.65 (dd, J = 2.0, 8.4 Hz, 1 H), 5.05 (dd, J = 5.6, 12.8 Hz, 1 H), 4.38 (s, 2 H), 4.16-4.03 (m, 4 H), 4.02-3.93 (m, 1 H), 3.86 (m, 3 H), 2.94-2.82 (m, 1 H), 2.63-2.51 (m, 3 H), 2.20 (s, 3 H), 2.06-1.95 (m, 1 H), 1.84-1.69 (m, 4 H), 1.68-1.55 (m, 2 H), 1.54-1.40 (m, 3 H), 0.75-0.63 (m, 2 H), 0.50-0.39 (m, 2 H) |
| Example 345 | | |
| 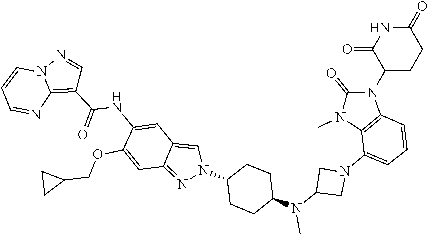 [SMILES: Cn1c(=O)n(C2CCC(=O)NC2=O)c2cccc(N3CC(N(C)[C@H]4CC[C@H](n5cc6cc(NC(=O)c7cnn8cccnc78)c(OCC7CC7)cc6n5)CC4)C3)c21] | 722.4 | DMSO-d₆ δ = 12.95 (s, 1H), 10.95 (s, 1H), 8.74-8.46 (m, 2H), 8.16 (s, 1H), 8.09 (s, 1H), 7.95 (s, 1H), 7.75-7.56 (m, 3H), 7.52 (d, J = 8.4 Hz, 1H), 7.27 (t, J = 8.4 Hz, 1H), 7.15-6.96 (m, 4H), 5.44-5.18 (m, 1H), 5.05 (dd, J = 4.8, 13.2 Hz, 1H), 4.63 (d, J = 1.2 Hz, 1H), 4.46-4.12 (m, 3H), 3.48 (s, 2H), 3.40 (s, 2H), 2.89 (s, 3H), 2.74-2.65 (m, 6H), 2.41-2.28 (m, 2H), 2.20-1.91 (m, 6H), 1.75-1.55 (m, 2H), 1.54-1.35 (m, 3H) |
| Example 346 | | |
| 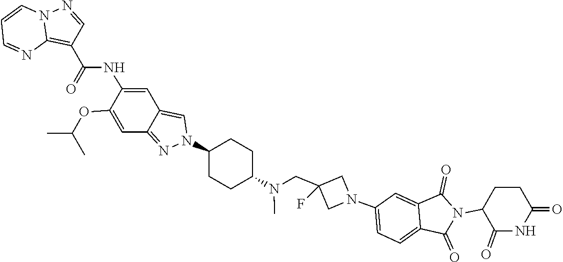 [SMILES: CC(C)Oc1cc2nn([C@H]3CC[C@H](N(C)CC4(F)CN(c5ccc6c(c5)C(=O)N(C5CCC(=O)NC5=O)C6=O)C4)CC3)cc2cc1NC(=O)c1cnn2cccnc12] | 791.2 | DMSO-d₆ δ = 11.07 (br s, 1H), 10.58 (s, 1H), 9.38 (d, J = 6.8 Hz, 1H), 8.87 (d, J = 4.0 Hz, 1H), 8.73 (s, 1H), 8.70 (s, 1H), 8.24 (s, 1H), 7.69 (d, J = 8.2 Hz, 1H),7.33 (dd, J = 4.2, 6.8 Hz, 1H), 7.12 (s, 1H), 6.92 (s, 1H), 6.77 (br d, J = 8.4 Hz, 1H), 5.07 (dd, J = 5.4, 12.8 Hz, 1H), 4.84 (td, J = 5.8, 12.0 Hz, 1H), 4.38-4.28 (m, 1H), 4.23-4.06 (m, 4H), 3.01-2.91 (m, 2H), 2.90-2.82 (m, 1H), 2.56 (br dd, J = 3.2, 9.6 Hz, 2H), 2.36 (s,3H), 2.17 (br d, J = 8.4 Hz, 2H), 2.09-1.95 (m, 2H), 1.91 (br d, J = 11.4 Hz, 4H), 1.58-1.49 (m, 2H), 1.46 (d, J = 5.8 Hz, 6H) |
| Example 347 | | |
| 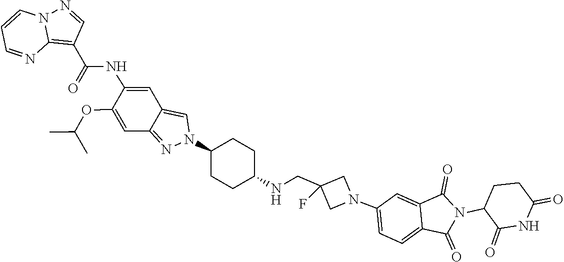 [SMILES: CC(C)Oc1cc2nn([C@H]3CC[C@H](NCC4(F)CN(c5ccc6c(c5)C(=O)N(C5CCC(=O)NC5=O)C6=O)C4)CC3)cc2cc1NC(=O)c1cnn2cccnc12] | 777.1 | DMSO-d₆ δ = 11.08 (s, 1H), 10.58 (s, 1H), 9.40-9.35 (m, 1H), 8.86 (dd, J = 1.2, 4.0 Hz, 1H), 8.73 (s, 1H), 8.70 (s, 1H), 8.26 (s, 1H), 7.68 (d, J = 8.4 Hz, 1H), 7.33 (dd, J = 4.4, 7.2 Hz, 1H), 7.12 (s, 1H), 6.89 (s, 1H), 6.75 (dd, J = 2.0, 8.4 Hz, 1H), 5.07 (dd, J = 5.4, 12.8 Hz, 1H), 4.84 (td, J = 6.0, 12.1 Hz, 1H), 4.40-4.30 (m, 1H), 4.25-4.07 (m, 4H), 3.10-3.00 (m, 2H), 2.95-2.82 (m, 1H), 2.63-2.53 (m, 3H), 2.16-1.98 (m, 6H), 1.97-1.84 (m, 2H), 1.47 (d, J = 6.0 Hz, 6H), 1.33-1.20 (m, 2H) |
| Example 348 | | |
| 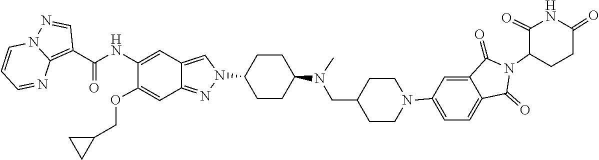 [SMILES: CN(CC1CCN(c2ccc3c(c2)C(=O)N(C2CCC(=O)NC2=O)C3=O)CC1)[C@H]1CC[C@H](n2cc3cc(NC(=O)c4cnn5cccnc45)c(OCC4CC4)cc3n2)CC1] | 831.4 | DMSO-d₆ δ = 11.08 (s, 1 H), 10.64 (s, 1 H), 9.38 (dd, J = 6.8, 1.6 Hz, 1 H), 8.86 (dd, J = 4.0, 1.6 Hz, 1 H), 8.74 (s, 1 H), 8.71 (s, 1H), 8.25 (s, 1 H), 7.66 (d, J = 8.8 Hz, 1 H), 7.29-7.35 (m, 2 H), 7.26 (d, J = 8.4 Hz, 1H), 7.02 (s, 1 H), 5.07 (dd, J = 13.2, 5.6 Hz, 1 H), 4.36 (s, 1 H), 4.08 (d, J = 12.8 Hz, 2 H), 4.01 (d, J = 6.8 Hz, 2 H), 3.00 (t, J = 12.4 Hz, 2 H), 2.84-2.93 (m, 1 H), |
| Example 349 | | 2.51-2.65 (m, 8 H), 2.18 (s, 2 H), 1.80- |
| | | 2.05 (m, 8 H), 1.43-1.67 (m, 3 H), 1.22 |
| | | (d, J = 11.2 Hz, 2 H), 0.66-0.75 |
| | | (m, 2 H), 0.45 (q, J = 4.8 Hz, 2 H) |
| 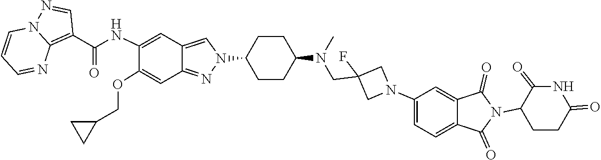 [SMILES: CN(CC1(F)CN(c2ccc3c(c2)C(=O)N(C2CCC(=O)NC2=O)C3=O)C1)[C@H]1CC[C@H](n2cc3cc(NC(=O)c4cnn5cccnc45)c(OCC4CC4)cc3n2)CC1] | 803.4 | DMSO-d₆ δ = 11.08 (s, 1H), 10.64 (s, 1H), 9.38 (dd, J = 1.6, 7.2 Hz, 1H), 8.87 (dd, J = 1.6, 4.4 Hz, 1H), 8.73 (d, J = 9.2 Hz, 2H), 8.25 (s, 1H), 7.70 (d, J = 8.0 Hz, 1H), 7.32 (dd, J = 4.4, 6.8 Hz, 1H), 7.03 (s, 1H), 6.93 (d, J = 1.6 Hz, 1H), 6.78 (dd, J = 2.0, 8.4 Hz, 1H), 5.08 (dd, J = 5.2, 12.8 Hz, 1H), 4.39-4.29 (m, 1H), 4.25-4.07 (m, 4H), 4.02 (d, J = 7.2 Hz, 2H), 3.03-2.93 (m, 2H), |
| Example 350 | | 2.91-2.84 (m, 1H), 2.62-2.58 (m, 1H), |
| | | 2.58-2.55 (m, 1H), 2.53 (br d, J = 2.0 |
| | | Hz, 1H), 2.38 (s, 3H), 2.22-2.14 (m, |
| | | 2H), 2.07-1.99 (m, 1H), 1.97-1.86 (m, |
| | | 4H), 1.59-1.46 (m, 3H), 0.74-0.69 (m, |
| | | 2H), 0.49-0.44 (m, 2H) |
| 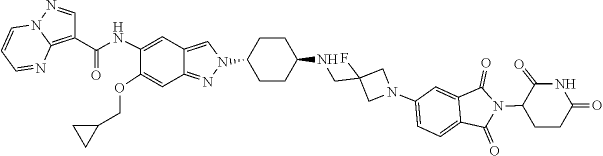 [SMILES: O=C1CCC(N2C(=O)c3ccc(N4CC(F)(CN[C@H]5CC[C@H](n6cc7cc(NC(=O)c8cnn9cccnc89)c(OCC8CC8)cc7n6)CC5)C4)cc3C2=O)C(=O)N1] | 789.3 | DMSO-d₆ δ = 11.09 (s, 1H), 10.65 (s, 1H), 9.38 (dd, J = 1.6, 7.2 Hz, 1H), 8.87 (dd, J = 1.6, 4.4 Hz, 1H), 8.73 (d, J = 11.6 Hz, 2H), 8.28 (s, 1H), 7.71 (d, J = 8.4 Hz, 1H), 7.35-7.30 (m, 1H), 7.03 (s, 1H), 6.93 (d, J = 2.0 Hz, 1H), 6.78 (dd, J = 2.0, 8.4 Hz, 1H), 5.09 (dd, J = 5.2, 12.8 Hz, 1H), 4.44-4.35 (m, 1H), 4.32-4.15 (m, 4H), 4.02 (d, J = 6.8 Hz, 2H), 2.96-2.75 (m, 2H), 2.63-2.57 |
| Example 351 | | (m, 1H), 2.55 (br t, J = 4.4 Hz, 1H), |
| | | 2.54-2.52 (m, 1H), 2.50-2.44 (m, 1H), |
| | | 2.16 (br s, 4H), 2.10-1.97 (m, 2H), |
| | | 1.97-1.84 (m, 2H), 1.56-1.34 (m, 3H), |
| | | 0.74-0.69 (m, 2H), 0.46 (q, J = 4.8 Hz, |
| | | 2H) |
| 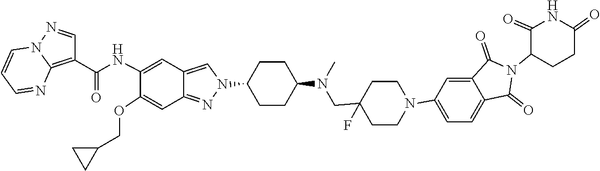 [SMILES: CN(CC1(F)CCN(c2ccc3c(c2)C(=O)N(C2CCC(=O)NC2=O)C3=O)CC1)[C@H]1CC[C@H](n2cc3cc(NC(=O)c4cnn5cccnc45)c(OCC4CC4)cc3n2)CC1] | 831.4 | DMSO-d₆ δ = 11.08 (s, 1H), 10.64 (s, 1H), 9.38 (dd, J = 1.6, 7.2 Hz, 1H), 8.87 (dd, J = 1.6, 4.0 Hz, 1H), 8.73 (d, J = 8.4 Hz, 2H), 8.24 (s, 1H), 7.69 (d, J = 8.4 Hz, 1H), 7.39 (s, 1H), 7.35-7.28 (m, 2H), 7.03 (s, 1H), 5.08 (dd, J = 5.2, 12.8 Hz, 1H), 4.37-4.28 (m, 1H), 4.02 (d, J = 6.8 Hz, 2H), 3.91 (br d, J = 13.2 Hz, 2H), 3.31-3.25 (m, 2H), 2.95-2.84 (m, 1H), 2.67-2.60 (m, 2H), 2.60-2.54 (m, 2H), 2.53 (br s, 1H), 2.35 (s, 3H), |
| Example 352 | | 2.16 (br d, J = 10.8 Hz, 2H), 2.06-2.00 |
| | | (m, 1H), 1.96-1.83 (m, 6H), 1.81-1.65 |
| | | (m, 2H), 1.55-1.44 (m, 3H), 0.74-0.69 |
| | | (m, 2H), 0.46 (q, J = 4.8 Hz, 2H). |
| 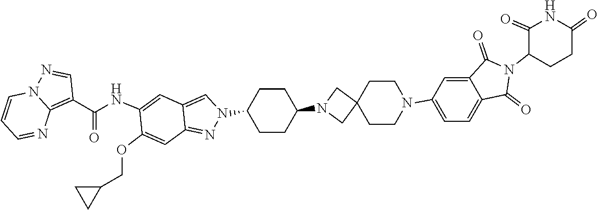 [SMILES: O=C1CCC(N2C(=O)c3ccc(N4CCC5(CC4)CN([C@H]4CC[C@H](n6cc7cc(NC(=O)c8cnn9cccnc89)c(OCC8CC8)cc7n6)CC4)C5)cc3C2=O)C(=O)N1] | 811.3 | DMSO-d₆ δ = 11.08 (s, 1H), 10.64 (s, 1H), 9.38 (dd, J = 1.6, 7.2 Hz, 1H), 8.87 (dd, J = 1.6, 4.0 Hz, 1H), 8.73 (d, J = 8.4 Hz, 2H), 8.24 (s, 1H), 7.69 (d, J = 8.4 Hz, 1H), 7.39 (s, 1H), 7.35-7.28 (m, 2H), 7.03 (s, 1H), 5.08 (dd, J = 5.2, 12.8 Hz, 1H), 4.37-4.28 (m, 1H), 4.02 (d, J = 6.8 Hz, 2H), 3.91 (br d, J = 13.2 Hz, 2H), 3.31-3.25 (m, 2H), 2.95-2.84 (m, 1H), 2.67-2.60 (m, 2H), 2.60-2.54 (m, 2H), 2.53 (br s, 1H), 2.35 (s, 3H), 2.16 (br d, J = 10.8 Hz, 2H), 2.06-2.00 (m, 1H), 1.96-1.83 (m, 6H), 1.81-1.65 |
| Example 353 | | (m, 2H), 1.55-1.44 (m, 3H), 0.74-0.69 |
| | | (m, 2H), 0.46 (q, J = 4.8 Hz, 2H). |
| 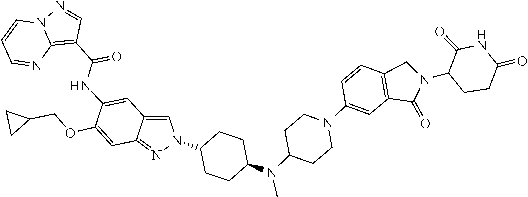 | 785.4 | DMSO-d₆ δ = 10.98 (s, 1H), 10.65 (s, 1H), 9.38 (dd, J = 1.6, 7.2 Hz, 1H), 8.87 (dd, J = 1.6, 4.4 Hz, 1H), 8.74 (s, 1H), 8.71 (s, 1H), 8.26 (s, 1H), 8.19 (s, 1H), 7.43 (d, J = 8.4 Hz, 1H), 7.32 (dd, J = 4.4, 7.2 Hz, 1H), 7.27 (dd, J = 2.4, 8.4 Hz, lH),7.17(d, J = 2.0 Hz, 1H), 7.03 (s, 1H), 5.10 (dd, J = 5.2, 13.2 Hz, 1H), 4.40-4.18 (m, 3H), 4.02 (d, J = 7.2 Hz, 2H), 3.82 (br d, J = 12.4 Hz, 2H), 2.96-2.88 (m, 1H), 2.82-2.74 (m, 3H), 2.64-2.56 (m, 1H), 2.52 (d, J = 2.0 Hz, 1H), 2.38 (br dd, J = 4.4, 12.8 Hz, 1H), |
| Example 354 | | 2.30 (s, 3H), 2.21-2.13 (m, 2H), 2.02- |
| | | 1.83 (m, 7H), 1.68-1.55 (m, 4H), 1.50 |
| | | (ddd, J = 5.2, 7.6, 12.4 Hz, 1H), 0.74- |
| | | 0.68 (m, 2H), 0.46 (q, J = 4.4 Hz, 2H) |
| 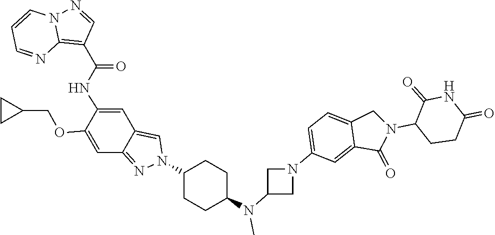 | 757.0 | DMSO-d₆ δ = 10.97 (br s, 1H), 10.64 (s, 1H), 9.37 (dd, J = 1.6, 7.2 Hz, 1H), 8.86 (dd, J = 1.6, 4.0 Hz, 1H), 8.72 (d, J = 11.2 Hz, 2H), 8.25 (s, 1H), 7.39 (d, J = 8.8 Hz, 1H), 7.34-7.28 (m, 1H), 7.03 (s, 1H), 6.74-6.70 (m, 2H), 5.09 (dd, J = 5.2, 13.2 Hz, 1H), 4.39-4.30 (m, 2H), 4.24-4.16 (m, 1H), 4.05-3.99 (m, 4H), 3.84-3.78 (m, 1H), 3.62 (br t, J = 6.4 Hz, 2H), 2.96-2.86 (m, 1H), 2.62-2.56 (m, 2H), 2.37 (br dd, J = 4.4, 12.8 Hz, 1H), 2.22-2.13 (m, 5H), 2.03-1.88 (m, 3H), 1.81 (br d, J = 12.0 Hz, 2H), 1.60-1.47 (m, 3H), 0.73-0.67 (m, 2H), 0.49-0.42 (m, 2H) |
| Example 355 | | |
| 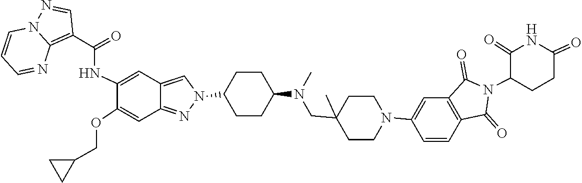 | 827.4 | DMSO-d₆ δ = 11.07 (s, 1H), 10.63 (s, 1H), 9.37 (dd, J = 1.4, 6.8 Hz, 1H), 8.86 (dd, J = 1.4, 4.2 Hz, 1H), 8.72 (d, J = 8.4 Hz, 2H), 8.23 (s, 1H), 7.64 (d, J = 8.6 Hz, 1H), 7.34-7.28 (m, 2H), 7.22 (dd, J = 1.8, 8.6 Hz, 1H), 7.01 (s, 1H), 5.06 (dd, J = 5.4, 12.8 Hz, 1H), 4.40-4.23 (m, 1H), 4.01 (d, J = 6.8 Hz, 2H), 3.78-3.71 (m, 2H), 3.25 (br s, 2H), 2.94-2.81 (m, 1H), 2.62-2.55 (m, 2H), 2.45-2.38 (m, 1H), 2.28 (s, 3H), 2.14 (br d, J = 10.0 Hz, 2H), 2.06-1.97 |
| Example 356 | | (m, 1H), 1.95-1.78 (m, 5H), 1.61-1.44 |
| | | (m, 6H), 1.34 (br d, J = 13.4 Hz, 2H), |
| | | 0.98 (s, 3H), 0.73-0.67 (m, 2H), 0.45 |
| | | (q, J = 4.6 Hz, 2H) |
| 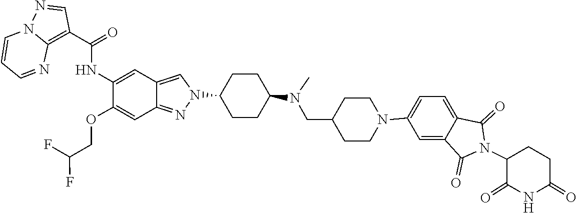 | 823.3 | DMSO-d₆ δ = 11.10-11.05 (m, 1H), 10.58 (s, 1H), 9.38 (dd, J = 1.4, 7.0 Hz, 1H), 8.86 (dd, J = 1.6, 4.0 Hz, 1H), 8.76 (s, 1H), 8.73-8.68 (m, 1H), 8.32 (s, 1H), 8.29 (s, 1H), 7.65 (d, J = 8.6 Hz, 1H), 7.36 (dd, J = 4.4, 6.8 Hz, 1H), 7.31 (d, J = 1.6 Hz, 1H), 7.26-7.22 (m, 1H), 7.21 (s, 1H), 6.81-6.46 (m, 1H), 5.06 (dd, J = 5.2, 12.8 Hz, 1H), 4.51 (dt, J = 3.4, 14.0 Hz, 2H), 4.39-4.30 (m, 1H), 4.05 (br d, J = 12.6 Hz, 2H), 2.98 (br t, J = 12.4 Hz, 2H), 2.90-2.83 (m, 1H), 2.62-2.57 (m, 2H), 2.27 (br d, J = 7.0 Hz, 2H), 2.24 (s, 3H), 2.20-2.11 |
| Example 357 | | (m, 2H), 2.05-1.97 (m, 1H), 1.96-1.71 |
| | | (m, 8H), 1.54-1.42 (m, 2H), 1.20-1.08 |
| | | (m, 2H) |
| 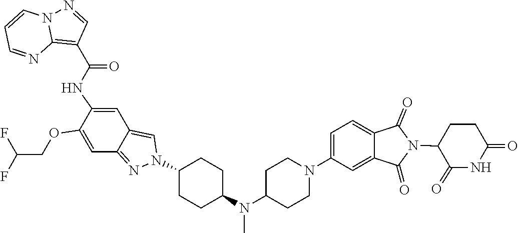 [SMILES: CN(C1CCN(c2ccc3c(c2)C(=O)N(C2CCC(=O)NC2=O)C3=O)CC1)[C@H]1CC[C@H](n2cc3cc(NC(=O)c4cnn5cccnc45)c(OCC(F)F)cc3n2)CC1] | 809.3 | DMSO-d₆ δ = 11.08 (s, 1 H) 10.59 (s, 1 H) 9.35-9.43 (m, 1 H) 8.86 (d, J = 4.0 Hz, 1 H) 8.76 (s, 1 H) 8.71 (s, 1 H) 8.30 (s, 1 H) 8.25 (s, 1 H) 7.66 (d, J = 8.4 Hz, 1 H) 7.36 (dd, J = 6.8, 4.0 Hz, 1 H) 7.33 (s, 1 H) 7.25 (br d, J = 8.8 Hz, 1 H) 7.21 (s, 1 H) 6.46-6.82 (m, 1 H) 5.07 (dd, J = 13.2, 5.6 Hz, 1 H) 4.46-4.56 (m, 2 H) 4.32-4.42 (m, 1 H) 4.08 (br d, J = 12.4 Hz, 2 H) 3.01 (br t, J = 10.8 Hz, 2 H) 2.80-2.91 (m, 2 H) 2.65-2.77 (m, 2 H) 2.61 (br s, 1 H)2.21 (s, 3 H) 2.17 (m, 2 H) 1.92-2.04 (m, 3 H) 1.79-1.90 (m, 4 H) 1.49-1.66 (m, 4H) |
| Example 358 | | |
| 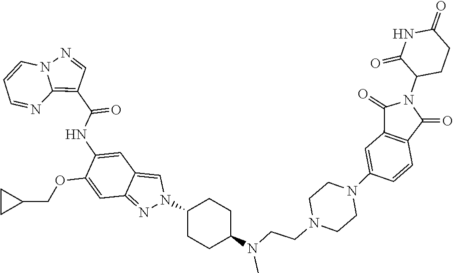 [SMILES: CN(CCN1CCN(c2ccc3c(c2)C(=O)N(C2CCC(=O)NC2=O)C3=O)CC1)[C@H]1CC[C@H](n2cc3cc(NC(=O)c4cnn5cccnc45)c(OCC4CC4)cc3n2)CC1] | 813.4 | DMSO-d₆ δ = δ 11.07 (s, 1 H), 10.63 (s, 1H),9.37 (dd, J = 7.2, 1.2 Hz, 1 H), 8.86 (dd, J = 4.4, 1.6 Hz, l H),8.72(d, J = 10.0 Hz, 2 H), 8.24 (s, 1 H), 7.68 (d, J = 8.4 Hz, 1 H), 7.29-7.36 (m, 2 H), 7.26 (dd, J = 8.8,1.69 Hz, 1 H), 7.02 (s, 1 H), 5.07 (dd, J = 12.8, 5.2 Hz, 1 H), 4.34 (t, J = 11.6 Hz, 1 H), 4.01 (d, J = 6.8 Hz, 2 H), 3.44 (s, 3 H),2.82-2.91 (m, 1 H), 2.53-2.65 (m, 10 H), 2.44-2.48 (m, 1 H), 2.27 (s, 3 H), 2.16 (d, J = 10.0 Hz, 2 H), 1.98-2.05 (m, 1 H), 1.83-1.95 (m, 4 H), 1.44-1.56 (m, 3 H), 0.66-0.75 (m, 2 H), 0.45 (q, J = 4.8 Hz, 2 H) |
| Example 359 | | |
| 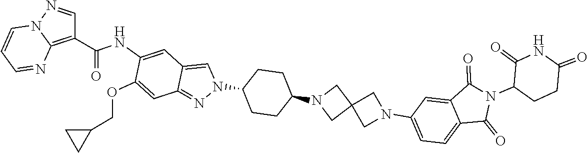 [SMILES: O=C1CCC(N2C(=O)c3ccc(N4CC5(C4)CN([C@H]4CC[C@H](n6cc7cc(NC(=O)c8cnn9cccnc89)c(OCC8CC8)cc7n6)CC4)C5)cc3C2=O)C(=O)N1] | 783.3 | DMSO-d₆ δ = 11.07 (s, 1 H), 10.64 (s, 1 H), 9.37 (d, J = 7.2 Hz, 1H), 8.86 (d, J = 2.8 Hz, 1 H), 8.72 (d, J = 9.2 Hz, 2 H), 8.26 (s, 1 H), 8.20 (s, 1 H), 7.64 (d, J = 8.0 Hz, 1 H), 7.31 (m, 1 H), 7.03 (s, 1 H), 6.79 (s, 1 H), 6.65 (d, J = 8.8 Hz, 1 H), 5.06 (m, 1 H), 4.33 (s, 1 H), 4.11 (s, 4 H), 4.01 (d, J = 7.2 Hz, 2H),2.89 (d, J = 13.6 Hz, 1 H), 2.53-2.62 (m, 4 H), 2.41-2.48 (m, 2 H), 1.98-2.15 (m, 4 |
| Example 360 | | H), 1.86 (d, J = 11.2 Hz, 4 H), 1.49 (s, |
| | | 1 H), 1.15 (d, J = 12.4 Hz, 2 H), 0.70 |
| | | (d, J = 8.4 Hz, 2 H), 0.45 (d, J = 4.4 |
| | | Hz, 2 H) |
| 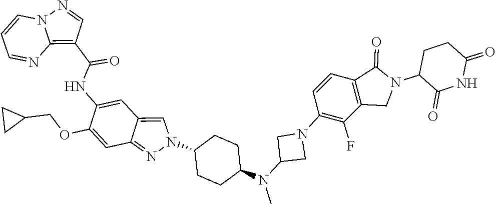 [SMILES: CN(C1CN(c2ccc3c(c2F)CN(C2CCC(=O)NC2=O)C3=O)C1)[C@H]1CC[C@H](n2cc3cc(NC(=O)c4cnn5cccnc45)c(OCC4CC4)cc3n2)CC1] | 775.3 | DMSO-d₆ δ = 10.84-11.05 (m, 1H), 10.64 (s, 1H), 9.36 (dd, J = 6.8, 1.6 Hz, 1H), 8.86 (dd, J = 4.4, 1.6 Hz, 1H), 8.74 (s, 1H), 8.71 (s, 1H), 8.39 (s, 1H), 8.25 (s, 1H), 7.38 (d, J = 8.0 Hz, 1H), 7.26-7.33 (m, 1H), 7.02 (s, 1H), 6.64 (t, J = 8.0 Hz, 1H), 5.04 (dd, J = 13.2, 5.2 Hz, 1H), 4.51-4.40 (m, 1H), 4.39-4.23 (m, 2H), 4.19-4.10 (m, 2H), 4.02 (d, J = 7.2 Hz, 2H), 3.88-3.79 (m, 3H), 2.98-2.82 (m, 1H), 2.62-2.55 (m, 2H), 2.44-2.32 (m, 1H), 2.22-2.12 (m, 5H), 2.00-1.85 (m, 3H), 1.84-1.74 (m, 2H), |
| Example 361 | | 1.59 -1.45 (m, 3H), 0.77-0.65 |
| | | (m, 2H), 0.49-0.40 (m, 2H) |
| 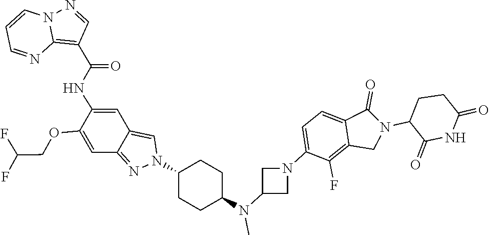 [SMILES: CN(C1CN(c2ccc3c(c2F)CN(C2CCC(=O)NC2=O)C3=O)C1)[C@H]1CC[C@H](n2cc3cc(NC(=O)c4cnn5cccnc45)c(OCC(F)F)cc3n2)CC1] | 785.2 | DMSO-d₆ δ = 10.97 (br s, 1H), 10.60 (s, 1H), 9.39 (dd, J = 1.6, 7.2 Hz, 1H), 8.86 (dd, J = 1.6, 4.4 Hz, 1H), 8.81-8.68 (m, 2H), 8.31 (s, 1H), 7.40 (d, J = 8.0 Hz, 1H), 7.38-7.35 (m, 1H), 7.22 (s, 1H), 6.80-6.50 (m, 2H), 5.06 (dd, J = 5.2, 13.2 Hz, 1H), 4.53 (dt, J = 3.6,14.0 Hz, 2H), 4.45 (d, J = 16.8 Hz, 1H), 4.38 (ddd, J = 3.6, 8.0, 11.6 Hz, 1H), 4.28 (d, J = 16.8 Hz, 1H), 4.16 (br s, 2H), 3.89-3.80 (m, 3H), 2.96-2.86 (m, 1H), 2.59 (br dd, J = 2.8, 13.2 Hz, 2H), 2.49-2.34 (m, 1H), 2.20 (s, 3H), 2.16 (br s, 2H), 2.01-1.90 (m, 3H), 1.85-1.77 (m, 2H), 1.62-1.50 (m, 2H) |
| Example 362 | | |
| 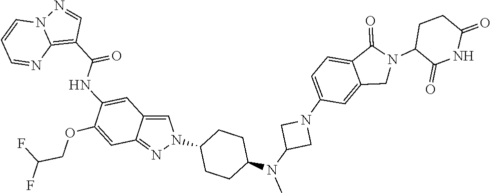 [SMILES: CN(C1CN(c2ccc3c(c2)CN(C2CCC(=O)NC2=O)C3=O)C1)[C@H]1CC[C@H](n2cc3cc(NC(=O)c4cnn5cccnc45)c(OCC(F)F)cc3n2)CC1] | 767.3 | DMSO-d₆ δ = 10.59 (s, 1 H) 9.38 (dd, J = 7.2, 1.6 Hz, 1 H) 8.86 (dd, J = 4.4, 1.6 Hz, 1 H) 8.77 (s, 1 H) 8.71 (s, 1 H) 8.41 (br s, 1 H) 8.31 (s, 1 H) 7.50 (d, J = 8.0 Hz, 1 H) 7.36 (dd, J = 6.8, 4.4 Hz, 1 H) 7.21 (s, 1 H) 6.53-6.79 (m, 2 H) 6.48-6.52 (m, 1 H) 5.04 (dd, J = 13.2, 5.2 Hz, 1 H) 4.52 (td, J = 14.00, 3.6 Hz, 2 H) 4.34-4.41 (m, 1 H) 4.29 (s, 1 H) 4.15-4.23 (m, 1 H) 4.03 (br t, J = 7.2 Hz, 2 H) 3.81-3.89 (m, 1 H) 3.72 (br t, J = 5.6 Hz, 2H) 2.83-2.96 |
| Example 363 | | (m, 1 H) 2.54-2.65 (m, 3 H) 2.33-2.42 |
| | | (m, 1 H) 2.13-2.24 (m, 5 H) 1.88-2.01 |
| | | (m, 3 H) 1.74-1.85 (m, 2 H) 1.51-1.62 |
| | | (m, 2H) |
| 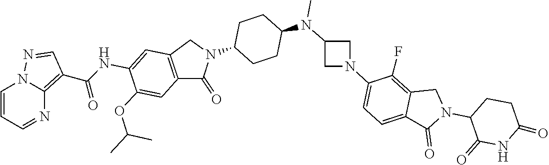 [SMILES: CC(C)Oc1cc2c(cc1NC(=O)c1cnn3cccnc13)CN([C@H]1CC[C@H](N(C)C3CN(c4ccc5c(c4F)CN(C4CCC(=O)NC4=O)C5=O)C3)CC1)C2=O] | 778.4 | DMSO-d₆ δ = 10.76 (s, 1H), 9.41 (dd, J = 1.6, 7.2 Hz, 1H), 8.90 (dd, J = 1.6, 4.4 Hz, 1H), 8.74 (s, 2H), 8.43 (s, 1H), 7.39 (d, J = 8.4 Hz, 1H), 7.38-7.35 (m, 1H), 7.34 (s, 1H), 6.65 (t, J = 8.0 Hz, 1H), 5.06 (dd, J = 5.2, 13.2 Hz, 1H), 4.90 (quin, J = 6.0 Hz, 1H), 4.45 (d, J = 16.8 Hz, 1H), 4.40 (s, 2H), 4.28 (d, J = 16.8 Hz, 1H), 4.15 (br s, 2H), 4.04-3.95 (m, 1H), 3.87-3.78 (m, 3H), 2.96- 2.86 (m, 1H), 2.59 (br dd, J = 2.4, 17.6 |
| | | Hz, 2H), 2.43-2.35 (m, 1H), 2.18 (s, |
| Example 364 | | 3H), 2.01-1.94 (m, 1H), 1.78 (br t, J = |
| | | 14.4 Hz, 4H), 1.69-1.59 (m, 2H), 1.55- |
| | | 1.46 (m, 2H), 1.45 (d, J = 6.0 Hz, 6H) |
| 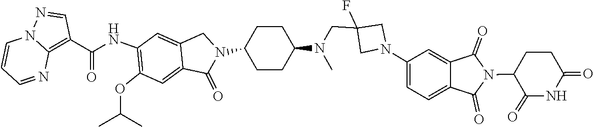 [SMILES: CC(C)Oc1cc2c(cc1NC(=O)c1cnn3cccnc13)CN([C@H]1CC[C@H](N(C)CC3(F)CN(c4ccc5c(c4)C(=O)N(C4CCC(=O)NC4=O)C5=O)C3)CC1)C2=O] | 806.3 | DMSO-d₆ δ = 11.10 (s, 1H), 10.75 (s, 1H), 9.40 (dd, J = 1.6, 7.2 Hz, 1H), 8.89 (dd, J = 1.6, 4.4 Hz, 1H), 8.73 (d, J = 3.2 Hz, 2H), 8.22 (s, 1H), 7.69 (d, J = 8.4 Hz, 1H), 7.38-7.34 (m, 1H), 7.32 (s, 1H), 6.92 (d, J = 2.0 Hz, 1H), 6.77 (dd, J = 2.0, 8.4 Hz, 1H), 5.08 (dd, J = 5.6, 13.2 Hz, 1H), 4.89 (td, J = 6.0, 12.4 Hz, 1H), 4.38 (s, 2H), 4.22-4.06 |
| Example 365 | | (m, 4H), 4.01-3.94 (m, 1H), 2.98-2.90 |
| | | (m, 2H), 2.89-2.83 (m, 1H), 2.59 (br |
| | | dd, J = 2.8, 14.0 Hz, 1H), 2.55 (br t, |
| | | J = 2.0Hz, 1H), 2.53 (d, J = 2.0 Hz, |
| | | 1H), 2.48-2.42 (m, 1H), 2.35 (s, 3H), |
| | | 2.06-1.99 (m, 1H), 1.81 (br t, J = 14.0 |
| | | Hz, 4H), 1.67-1.56 (m, 2H), 1.51-1.46 |
| | | (m, 1H), 1.44 (d, J = 6.0 Hz, 6H) |
| 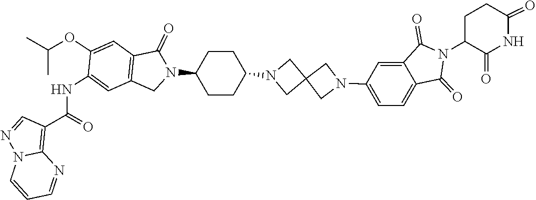 [SMILES: CC(C)Oc1cc2c(cc1NC(=O)c1cnn3cccnc13)CN([C@H]1CC[C@H](N3CC4(CN(c5ccc6c(c5)C(=O)N(C5CCC(=O)NC5=O)C6=O)C4)C3)CC1)C2=O] | 786.2 | DMSO-d₆ δ = 10.96 (br d, J = 16.0 Hz, 1H), 10.65 (d, J = 5.2 Hz, 1H),9.38(br d, J = 7.2 Hz, 1H), 8.87 (br s, 1H), 8.77-8.69 (m, 2H), 8.34-8.23 (m, 2H), 7.35-7.26 (m, 2H), 7.07-6.98 (m, 2H), 6.55 (br d, J = 8.0 Hz, 1H), 5.09 (dt, J = 5.2, 13.6 Hz, 1H), 4.50-4.28 (m, 3H), 4.05-3.99 (m, 2H), 3.75-3.64 (m, 5H), 2.96-2.87 (m, 1H), 2.67-2.59 (m, 2H), 2.59-2.54 (m, 2H), 2.48-2.35 (m, 3H), 2.20-2.13 (m, 1H), 2.00-1.86 (m, 4H), 1.85-1.75 (m, 1H), 1.66-1.48 (m, 7H), 0.71 (br d, J = 7.6 Hz, 2H), 0.46 (br d, |
| Example 366 | | J = 4.8 Hz, 2H) |
| 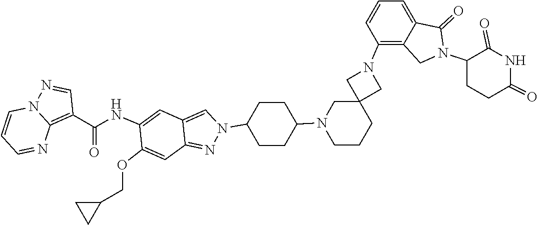 [SMILES: O=C1CCC(N2Cc3c(cccc3N3CC4(CCCN(C5CCC(n6cc7cc(NC(=O)c8cnn9cccnc89)c(OCC8CC8)cc7n6)CC5)C4)C3)C2=O)C(=O)N1] | 797.3 | DMSO-d₆ δ = 10.96 (br d, J = 16.0 Hz, 1H), 10.65 (d, J = 5.2 Hz, 1H), 9.38 (br d, J = 7.2 Hz, 1H), 8.87 (brs, 1H), 8.77- 8.69 (m, 2H), 8.34-8.23 (m, 2H), 7.35- 7.26 (m, 2H), 7.07-6.98 (m, 2H), 6.55 (br d, J = 8.0 Hz, 1H), 5.09 (dt, J = 5.2, 13.6 Hz, 1H), 4.50-4.28 (m, 3H), 4.05-3.99 (m, 2H), 3.75-3.64 (m, 5H), 2.96-2.87 (m, 1H), 2.67-2.59 (m, 2H), 2.59-2.54 (m, 2H), 2.48-2.35 (m, 3H), 2.20-2.13 (m, 1H), 2.00-1.86 (m, 4H), 1.85-1.75 (m, 1H), 1.66-1.48 (m, 7H), 0.71 (br d, J = 7.6 Hz, 2H), 0.46 (br d, J = 4.8 Hz, 2H) |
| Example 367 | | |
| 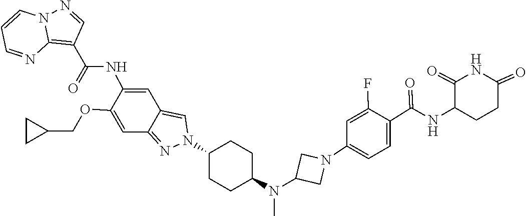 [SMILES: CN(C1CN(c2ccc(C(=O)NC3CCC(=O)NC3=O)c(F)c2)C1)[C@H]1CC[C@H](n2cc3cc(NC(=O)c4cnn5cccnc45)c(OCC4CC4)cc3n2)CC1] | 786.2 | DMSO-d₆ δ = 10.85 (br s, 1H), 10.65 (s, 1H), 9.38 (dd, J = 1.6, 7.2 Hz, 1H), 8.87 (dd, J = 1.2, 4.0 Hz, 1H), 8.78-8.73 (m, 1H), 8.73-8.69 (m, 1H), 8.28-8.25 (m, 1H), 8.25-8.23 (m, 1H), 8.00 (t, J = 7.6 Hz, 1H), 7.63 (t, J = 8.8 Hz, 1H), 7.33 (dd, J = 4.0, 6.8 Hz, 1H), 7.04 (s, 1H), 6.31 (dd, J = 2.0, 8.4 Hz, 1H), 6.28-6.23 (m, 1H), 4.78-4.68 (m, 1H), 4.40-4.30 (m, 1H), 4.07-3.99 (m, 4H), 3.89-3.81 (m, 1H), 3.78-3.73 (m, 2H), 2.82-2.73 (m, 1H), 2.64-2.57 (m, 1H), 2.26-1.86 (m, 10H), 1.85-1.75 (m, 2H), 1.63-1.45 (m, 3H), 0.75-0.68 (m, |
| Example 368 | | 2H), 0.51-0.43 (m, 2H) |
| 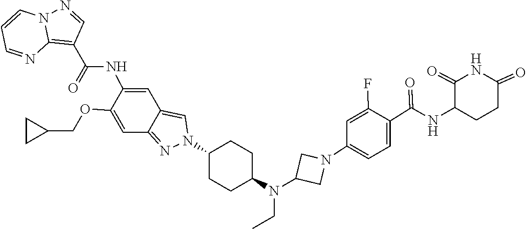 [SMILES: CCN(C1CN(c2ccc(C(=O)NC3CCC(=O)NC3=O)c(F)c2)C1)[C@H]1CC[C@H](n2cc3cc(NC(=O)c4cnn5cccnc45)c(OCC4CC4)cc3n2)CC1] | 777.2 | DMSO-d₆ δ = 10.85 (s, 1H), 10.65 (s, 1H), 9.7 (s, 1H), 9.37 (d, J = 7.2 Hz, 2H), 8.87-8.86 (m, 1H), 8.75 (s, 1H), 8.71 (s, 1H), 8.25 (s, 1H), 8.13-8.08 (m, 1H), 7.68-7.64 (m, 1H), 7.33-7.31 (m, 1H), 7.01 (s, 1H), 6.44-6.39 (m, 2H), 4.74-4.71 (m, 1H), 4.5-4.0 (m, 7H), 3.7-3.5 (m, 1H), 3.25-3.2 (m, 1H), 2.7-2.5 (m, 3H), 2.22-1.90 (m, 8H), 1.77-1.68 (m, 2H), 1.50-1.40 (m, 1H), 1.30-1.20 (m, 3H), 0.72-0.70 (m, 2H), 0.47-0.45 (m, 2H). |
| Example 369 | | |
| 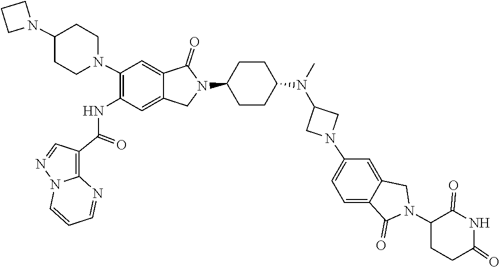 [SMILES: CN(C1CN(c2ccc3c(c2)CN(C2CCC(=O)NC2=O)C3=O)C1)[C@H]1CC[C@H](N2Cc3cc(NC(=O)c4cnn5cccnc45)c(N4CCC(N5CCC5)CC4)cc3C2=O)CC1] | 840.3 | DMSO-d₆ δ = 10.95 (s, 2H), 9.42 (dd, J = 1.6, 6.8 Hz, 1H), 8.98 (br d, J = 2.8 Hz, 1H), 8.76 (d, J = 8.0 Hz, 2H), 8.19 (s, 2H), 7.56 (s, 1H), 7.50 (d, J = 8.4 Hz, 1H), 7.45 (dd, J = 4.0, 6.8 Hz, 1H), 6.55 (s, 1H), 6.51 (dd, J = 1.6, 8.4 Hz, 1H), 5.05 (dd, J = 5.2, 13.2 Hz, 1H), 4.42 (s, 2H), 4.35-4.17 (m, 2H), 4.07- 4.01 (m, 2H), 4.01-3.95 (m, 1H), 3.87- 3.81 (m, 1H), 3.72 (br t,J = 5.6Hz, 2H), 3.33 (br t, J = 6.8 Hz, 4H), 3.00 (br d, J = 10.8 Hz, 2H), 2.96-2.86 (m, 1H), 2.75 (br t, J = 10.0 Hz, 2H), 2.63-2.58 (m, 2H), 2.45-2.37 (m, 2H), 2.20 (s, 3H), 2.10-2.03 (m, 2H), 2.00-1.93 (m, 1H), 1.89-1.74 (m, 6H), 1.70-1.59 (m, 4H), 1.55-1.44 (m, 2H) |
| Example 370 | | |
| 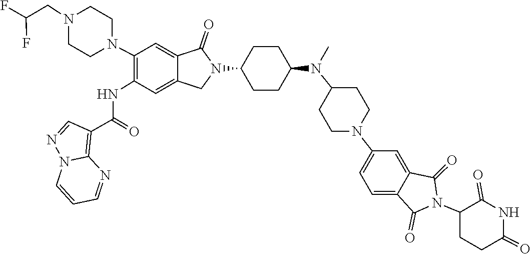 [SMILES: CN(C1CCN(c2ccc3c(c2)C(=O)N(C2CCC(=O)NC2=O)C3=O)CC1)[C@H]1CC[C@H](N2Cc3cc(NC(=O)c4cnn5cccnc45)c(N4CCN(CC(F)F)CC4)cc3C2=O)CC1] | 892.3 | DMSO-d₆ δ = 11.08 (br s, 1 H) 10.82 (s, 1 H) 9.39-9.43 (m, 1 H) 8.99 (d, J = 2.80 Hz, 1 H) 8.74-8.76 (m, 2 H) 8.22 (s, 1 H) 7.63-7.68 (m, 1 H) 7.53 (s, 1H) 7.39 (dd, J = 6.80, 4.40 Hz, 1 H) 7.32 (d, J = 1.60 Hz, 1 H) 7.24 (dd, J = 8.80, 1.60 Hz, 1 H) 6.06-6.38 (m, 1 H) 5.07 (dd, J = 12.80, 5.20 Hz, 1 H) 4.41 (s, 2 H) 4.07 (br d, J = 13.20 Hz, 2 H) 3.94-4.01 (m, 1 H) 3.00 (br t, J = 12.40 Hz, 3 H) 2.90 (br s, 6 H) 2.85 (br s, 5 H) 2.60 (br d, J = 2.00 Hz, 2 H) 2.20 (s, 3 H) 1.96-2.07 (m, 2 H) 1.74-1.84 (m, 6 H) 1.65 (br d, J = 10.80 Hz, 2 H) 1.45-1.57 (m, 4H) |
| Example 371 | | |
| 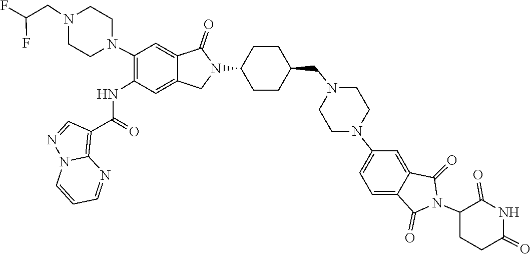 [SMILES: O=C1CCC(N2C(=O)c3ccc(N4CCN(C[C@H]5CC[C@H](N6Cc7cc(NC(=O)c8cnn9cccnc89)c(N8CCN(CC(F)F)CC8)cc7C6=O)CC5)CC4)cc3C2=O)C(=O)N1] | 878.3 | DMSO-d₆ δ 11.08 (s, 1H), 10.83 (s, 1 H), 9.40 (dd, J = 1.2, 7.2 Hz, 1 H), 9.00 (dd, J = 1.6, 4.4 Hz, 1 H), 8.75 (d, J = 4.4 Hz, 2 H), 7.68 (d, J = 8.4 Hz, 1 H), 7.54 (s, 1 H),7.40 (dd, J = 4.4, 7.2 Hz, 1 H), 7.34 (s, 1 H), 7.29-7.23 (m, 1H), 6.42-5.98 (m, 1 H), 5.07 (dd, J = 5.6, 12.8 Hz, 1 H), 4.43 (s, 2 H), 4.05-3.94 (m, 1 H), 3.45 (s, 4 H), 2.98-2.78 (m, 13 H), 2.63-2.53 (m, 3 H), 2.22-2.14 (m, 2H), 2.05-1.98 (m, 1 H), 1.94-1.86 (m, 2H), 1.79-1.73 (m, 2H), 1.70-1.48 (m, 4 H), 1.11-1.01 (m, 2H) |
| Example 371’ | | |
| 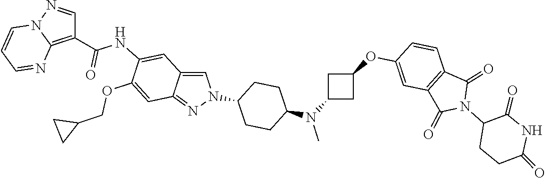 [SMILES: CN([C@H]1CC[C@H](n2cc3cc(NC(=O)c4cnn5cccnc45)c(OCC4CC4)cc3n2)CC1)[C@H]1C[C@H](Oc2ccc3c(c2)C(=O)N(C2CCC(=O)NC2=O)C3=O)C1] | 786.3 | DMSO-d₆ δ = 11.11 (br s, 1H), 10.63 (s, 1H), 9.37 (dd, J = 1.2, 6.8 Hz, 1H), 8.86 (dd, J = 1.6, 4.4 Hz, 1H), 8.71 (d, J = 8.4 Hz, 2H), 8.23 (s, 1H), 7.84 (d, J = 8.6 Hz, 1H), 7.35-7.23 (m, 3H), 7.01 (s, 1H), 5.12 (dd, J = 5.4, 13.0 Hz, 1H), 4.96 (br t, J = 6.2 Hz, 1H), 4.38-4.25 (m, 1H), 4.01 (d, J = 6.8 Hz, 2H), 3.55-3.46 (m, 2H), 2.89 (ddd, J = 5.4, 14.2, 17.4 Hz, 2H), 2.63-2.54 (m, 5H), 2.22 (br dd, J = 7.8 11.0, 2H), 2.12 (s, 3H), 2.09-2.00 (m, 1H), 1.97-1.84 |
| Example 372 | | (m, 2H), 1.76 (br d, J = 10.4 Hz, 2H), |
| | | 1.59-1.44 (m, 3H), 0.77-0.64 (m, 2H), |
| | | 0.45 (q, J = 4.6 Hz, 2H) |
| 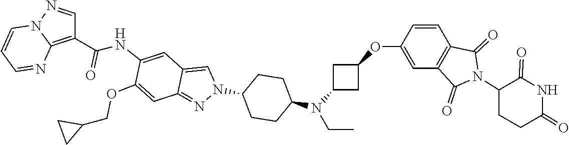 [SMILES: CCN([C@H]1CC[C@H](n2cc3cc(NC(=O)c4cnn5cccnc45)c(OCC4CC4)cc3n2)CC1)[C@H]1C[C@H](Oc2ccc3c(c2)C(=O)N(C2CCC(=O)NC2=O)C3=O)C1] | 800.3 | DMSO-d₆ δ = 11.11 (s, 1 H), 10.63 (s, 1 H), 9.37 (dd, J = 1.6, 7.2 Hz, 1 H), 8.89-8.83 (m, 1 H), 8.71 (d, J = 7.2 Hz, 2 H), 8.22 (s, 1 H), 7.85 (d, J = 8.4 Hz, 1 H), 7.34-7.23 (m, 3 H), 7.01 (s, 1 H), 5.12 (dd, J = 4.4, 12.8 Hz, 1 H), 4.99-4.90 (m, 1 H), 4.41-4.28 (m, 1H), 4.01 (d, J = 7.2 Hz, 2 H), 3.79-3.71 (m, 1 H), 2.95-2.80 (m, 1 H), 2.64-2.53 (m, 4 H), 2.36 (m, 3 H), 2.29-2.21 (m, 2 H), |
| Example 373 | | 2.18-2.09 (m, 2 H), 2.09-2.03 (m, 1 H), |
| | | 1.90 (m, 2 H), 1.84-1.74 (m, 2 H), |
| | | 1.57-1.43 (m, 3 H), 1.06 -0.96 (m, 3 |
| | | H), 0.76-0.64 (m, 2 H), 0.49-0.40 (m, 2 |
| | | H)) |
| 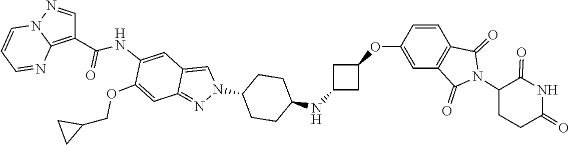 [SMILES: O=C1CCC(N2C(=O)c3ccc(O[C@H]4C[C@H](N[C@H]5CC[C@H](n6cc7cc(NC(=O)c8cnn9cccnc89)c(OCC8CC8)cc7n6)CC5)C4)cc3C2=O)C(=O)N1] | 772.2 | DMSO-d₆ δ = 11.11 (br s, 1H), 10.64 (s, 1H), 9.37 (dd, J = 1.6, 7.0 Hz, 1H), 8.86 (dd, J = 1.6, 4.2 Hz, 1H), 8.72 (d, J = 8.4 Hz, 2H), 8.27-8.24 (m, 1H), 8.23-8.20 (m, 1H), 7.84 (d, J = 8.6 Hz, 1H), 7.32 (dd, J = 4.4, 7.0 Hz, 1H), 7.29-7.23 (m, 2H), 7.02 (s, 1H), 5.16-5.02 (m, 2H), 4.35 (dt, J = 3.4, 11.4 Hz, 1H), 4.01 (d, J = 7.0 Hz, 2H), 3.63-3.58 (m, 2H), 2.96-2.82 (m, 2H), 2.63-2.56 |
| Example 374 | | (m, 4H), 2.14-1.82 (m, 8H), 1.56-1.43 |
| | | (m, 1H), 1.35-1.19 (m, 3H), 0.75-0.66 |
| | | (m, 2H), 0.49-0.41 (m, 2H) |
| 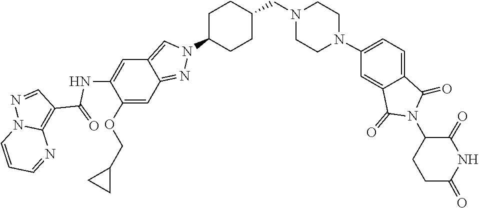 [SMILES: O=C1CCC(N2C(=O)c3ccc(N4CCN(C[C@H]5CC[C@H](n6cc7cc(NC(=O)c8cnn9cccnc89)c(OCC8CC8)cc7n6)CC5)CC4)cc3C2=O)C(=O)N1] | 785.4 | DMSO-d₆ δ = 11.08 (s, 1H), 10.64 (s, 1H), 9.37 (dd, J = 1.2, 7.2 Hz, 1H), 8.86 (d, J = 2.8 Hz, 1H), 8.73 (d, J = 11.6 Hz, 2H), 8.26 (s, 1H), 7.68 (d, J = 8.4 Hz, 1H), 7.38-7.29 (m, 2H), 7.26 (br d, J = 8.4 Hz, 1H), 7.03 (s, 1H), 5.07 (dd, J = 5.2, 12.8 Hz, 1H), 4.42- 4.26 (m, 1H), 4.06-3.99 (m, 2H), 3.45 (br s, 4H), 3.32-3.28 (m, 2H), 2.95- 2.81 (m, 1H), 2.63-2.53 (m, 2H), 2.21 (br d, J = 6.8 Hz, 2H), 2.18-2.09 (m, 2H), 2.05-1.82 (m, 6H), 1.73-1.59 (m, 1H), 1.56-1.42 (m, 1H), 1.22-1.05 (m, |
| Example 375 | | 3H), 0.77-0.64 (m, 2H), 0.51-0.41 (m, |
| | | 2H) |
| 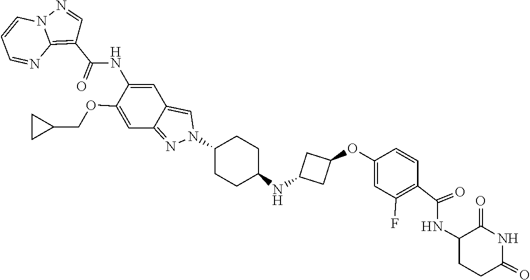 [SMILES: O=C1CCC(NC(=O)c2ccc(O[C@H]3C[C@H](N[C@H]4CC[C@H](n5cc6cc(NC(=O)c7cnn8cccnc78)c(OCC7CC7)cc6n5)CC4)C3)cc2F)C(=O)N1] | 763.8 | DMSO-d₆ δ = 10.86(s, 1H), 10.64 (s, 1H), 9.38 (dd, J = 1.6, 7.6 Hz, 1H), 8.86 (dd, J = 1.6, 4.0 Hz, 1H), 8.74 (s, 1H), 8.71 (s, 1H), 8.32 (dd, J = 4.0, 7.6 Hz, 1H), 8.26 (s, 1H), 7.73-7.66 (m, 1H), 7.32 (dd, J = 4.0, 7.2 Hz, 1H), 7.02 (s, 1H), 6.84-6.76 (m, 2H), 4.96 (s, 1H), 4.80-4.68 (m, 1H), 4.46-4.34 (m, 1H), 4.01 (d, J = 6.8 Hz, 2H), 3.94- 3.85 (m, 1H), 2.99-2.90 (m, 1H), 2.82 - 2.72 (m, 1H), 2.64 -2.54 (m, 3H), 2.42 (s, 3H), 2.18-2.06 (m, 5H), 2.02-1.89 (m, 3H), 1.56-1.36 (m, 3H), 0.76-0.65 (m, 2H), 0.50-0.40 (m, 2H) |
| Example 376 | | |

### Preparation of Example 377: N-(2-((1r,3r)-3-((1-(2-(2,6-dioxopiperidin-3-yl)-1,3-dioxoisoindolin-5-yl)piperidin-4-yl)(ethyl)amino)cyclobutyl)-6-isopropoxy-2H-indazol-5yl)pyrazolo[1,5-a]pyrimidine-3-carboxamide

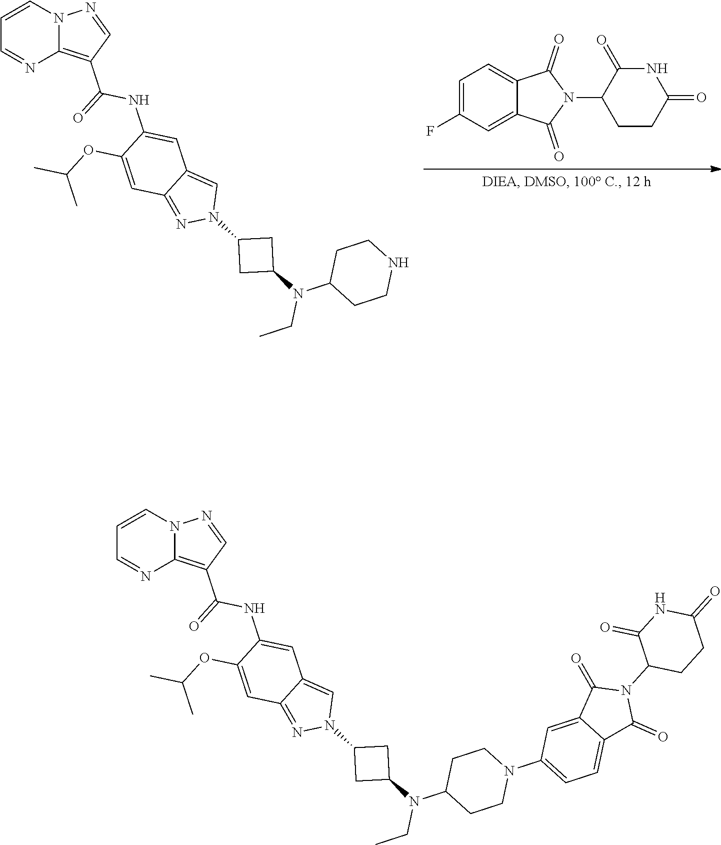 [SMILES: CCN(C1CCNCC1)[C@H]1C[C@H](n2cc3cc(NC(=O)c4cnn5cccnc45)c(OC(C)C)cc3n2)C1 ; O=C1CCC(N2C(=O)c3ccc(F)cc3C2=O)C(=O)N1 ; CCN(C1CCN(c2ccc3c(c2)C(=O)N(C2CCC(=O)NC2=O)C3=O)CC1)[C@H]1C[C@H](n2cc3cc(NC(=O)c4cnn5cccnc45)c(OC(C)C)cc3n2)C1]

To a solution of N-(2-((1r,3r)-3-(ethyl(piperidin-4-yl)amino)cyclobutyl)-6-isopropoxy-2H-indazol-5-yl)pyrazolo[1,5-a]pyrimidine-3-carboxamide (160 mg, 253.70 μmol, 1 eq, trifluoroacetate), prepared in an analogous synthetic sequence as described for Example 309 except commencing from tert-butyl ((1r,3r)-3-aminocyclobutyl)carbamate, in dimethyl sulfoxide (3 mL) was added N,N-diisopropylethylamine (327.89 mg, 2.54 mmol, 441.90 μL, 10 eq) and 2-(2,6-dioxopiperidin-3-yl)-5-fluoroisoindoline-1,3-dione (105.12 mg, 380.55 μmol, 1.5 eq). The mixture was stirred at 100° C. for 12 h. The mixture was poured into water (50 mL) and was extracted with dichloromethane (50 mL×2). The combined organic phase was washed with brine (30 mL×2), dried with anhydrous sodium sulfate, filtered, and concentrated in vacuo. The residue was purified by silica gel chromatography (eluted with a gradient from 0% to 4% methanol in dichloromethane) and further purified by semi-preparative reverse phase HPLC (column: Unisil 3-100 C18 Ultra 150×50 mm×3 um; mobile phase: [water (0.225% formic acid)-acetonitrile]; B %: 18%-48%, 10 min) to afford N-(2-((1r,3r)-3-((1-(2-(2,6-dioxopiperidin-3-yl)-1,3-dioxoisoindolin-5-yl)piperidin-4-yl)(ethyl)amino)cyclobutyl)-6-isopropoxy-2H-indazol-5-yl)pyrazolo[1,5-a]pyrimidine-3-carboxamide (94.6 mg, 113.21 μmol, 45% yield, 98% purity, formate) as a yellow solid. MS (ESI) m/z: 773.6 [M+H]⁺. ¹H NMR (400 MHz, DMSO-d₆) δ=11.08 (s, 1H), 10.60 (s, 1H), 9.39 (dd, J=1.6, 7.2 Hz, 1H), 8.87 (dd, J=1.6, 4.4 Hz, 1H), 8.74 (s, 1H), 8.71 (s, 1H), 8.35 (s, 1H), 8.15 (s, 1H), 7.65 (d, J=8.8 Hz, 1H), 7.36-7.31 (m, 2H), 7.24 (dd, J 2.0, 8.4 Hz, 1H), 7.17 (s, 1H), 5.06 (dd, J=5.6, 13.2 Hz, 1H), 5.02-4.95 (m, 1H), 4.86 (td, J=6.0, 12.0 Hz, 1H), 4.13 (br d, J=12.8 Hz, 2H), 3.95 (quin, J=7.6 Hz, 1H), 2.98-2.83 (m, 4H), 2.58 (br d, J=7.2 Hz, 8H), 2.05-1.96 (m, 1H), 1.74 (br d, J=11.6 Hz, 2H), 1.57-1.49 (m, 2H), 1.47 (d, J=6.0 Hz, 6H), 1.01 (t, J=7.2 Hz, 3H).

### Preparation of Example 378: N-(6-(cyclopropylmethoxy)-2-(1-((1-(1-(2,6-dioxopiperidin-3-yl)-3-methyl-2-oxo-2,3-dihydro-1H-benzo[d]imidazol-4-yl)azetidin-3-yl)methyl)piperidin-4-yl)-2H-indazol-5-yl)pyrazolo[1,5-a]pyrimidine-3-carboxamide

### Step 1:

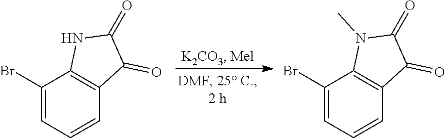

To a mixture of 7-bromoindoline-2,3-dione (12 g, 53.09 mmol, 1 eq), potassium carbonate (11.01 g, 79.64 mmol, 1.5 eq) and water (1.2 mL) in DMF (60 mL) was added dropwise a solution of methyl iodide (8.44 g, 59.43 mmol, 3.7 mL, 1.12 eq) in DMF (24 mL) The mixture was stirred at 25° C. for 2 h. Upon completion of the reaction, water (120 mL) was added and the mixture was stirred at 0° C. for 1 h. The resultant precipitate was collected by filtration, washed with water (50 mL×2), and dried in vacuo to yield 7-bromo-1-methylindoline-2,3-dione (8 g, 33.33 mmol, 62% yield) as a red solid, which was used in subsequent reactions without further purification. MS (ESI) m/z: 242.3 [M+H]⁺. ¹H NMR (400 MHz, DMSO-d₆) δ: 7.81 (br d, J=7.6 Hz, 1H) 7.55 (br d, J=7.2 Hz, 1H) 7.05 (t, J=8.0 Hz, 1H) 3.47 (s, 3H).

### Step 2:

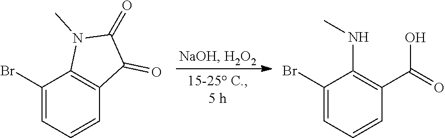

Aqueous hydrogen peroxide (38 g, 335.15 mmol, 32.20 mL, 30% purity, 10.06 eq) was added dropwise to a mixture of 7-bromo-1-methylindoline-2,3-dione (8 g, 33.33 mmol, 1 eq) and sodium hydroxide (2 M, 199.96 mL, 12 eq) below 15° C. The mixture was stirred at 25° C. for 5 h. After the pH was adjusted to 4.0 with hydrochloric acid (1 M), the mixture was stirred at 10° C. for 1 h. The mixture was then extracted with ethyl acetate (80 mL×3). The combined organic phase was washed with water (60 mL), dried with anhydrous sodium sulfate, filtered, and concentrated under vacuum to provide 3-bromo-2-(methylamino)benzoic acid (6.0 g, crude) as a brown oil which was used in the next step without further purification. MS (ESI) m/z: 252.2 [M+Na]⁺.

### Step 3:

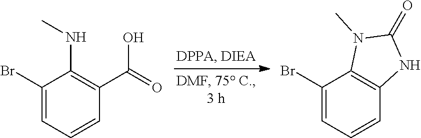

Diphenylphosphoryl azide (10.76 g, 39.10 mmol, 8.47 mL, 1.50 eq) was added dropwise to a stirred solution of 3-bromo-2-(methylamino)benzoic acid (6 g, 26.08 mmol, 1 eq, crude) and N,N-diisopropylethylamine (5.04 g, 39.01 mmol, 6.80 mL, 1.50 eq) in DMF (40 mL) at 75° C. The reaction mixture was stirred at 75° C. for 3 h. Water (30 mL) was added at 25° C., and the mixture was stirred at 0° C. for 0.5 h. The resultant precipitate was collected by filtration, washed with water (30 mL) and diisopropyl ether (15 mL), and dried in vacuo at 50° C. to afford 7-bromo-1-methyl-1H-benzo[d]imidazol-2(3H)-one (4 g, 17.62 mmol, 67% yield) as an off-white solid which was used in subsequent transformations without further purification. MS (ESI) m/z: 226.9 [M+H]⁺. ¹H NMR (400 MHz, DMSO-d₆) δ: 11.17 (br s, 1H) 7.14 (dd, J=8.0, 1.2 Hz, 1H) 6.96-6.99 (m, 1H) 6.88-6.93 (m, 1H) 3.55 (s, 3H).

### Step 4:

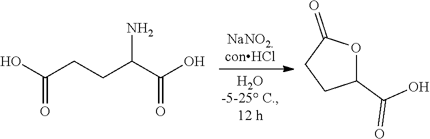 [SMILES: NC(CCC(=O)O)C(=O)O ; O=C1CCC(C(=O)O)O1]

To a solution of 2-aminopentanedioic acid (50 g, 339.84 mmol, 1 eq) in water (300 mL) and concentrated hydrochloric acid (37%, 50 mL) was added sodium nitrite (35.17 g, 509.76 mmol, 1.5 eq) at −5° C. The mixture was stirred at 25° C. for 12 h. The reaction was filtered, and the filtrate was concentrated in vacuo to yield 5-oxotetrahydrofuran-2-carboxylic acid (34 g, crude) as a colorless oil which was used for the next step without further purification. H NMR (400 MHz, CDCl₃) δ: 7.27 (s, 1H) 4.98-5.03 (m, 1H) 2.51-2.72 (m, 4H).

### Step 5:

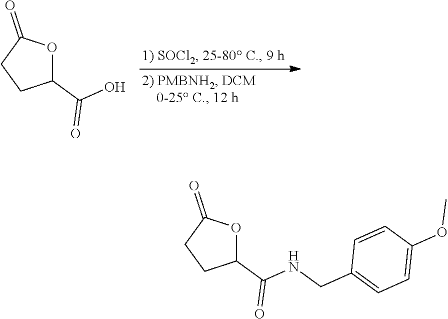 [SMILES: O=C1CCC(C(=O)O)O1 ; COc1ccc(CNC(=O)C2CCC(=O)O2)cc1]

To a solution of 5-oxotetrahydrofuran-2-carboxylic acid (67 g, 514.99 mmol, 1 eq, crude) in dichloromethane (400 mL) was added thionyl chloride (135 g, 1.13 mol, 82.32 mL, 2.20 eq) at 0° C. The mixture was stirred at 85° C. for 3 h and 25° C. for 6 h. The reaction mixture was concentrated in vacuo to give the crude product. The residue was then resuspended in DCM (400 mL) followed by the addition of p-methoxyaniline (56.52 g, 411.99 mmol, 53.32 mL, 0.8 eq), and triethylamine (104.22 g, 1.03 mol, 143.36 mL, 2 eq) at 0° C. The mixture was stirred at 25° C. for 12 h. The reaction mixture was diluted with water (500 mL) and the mixture was extracted with ethyl acetate (500 mL×3). The combined organic phase was washed with brine (200 mL), dried over anhydrous sodium sulfate, filtered and concentrated in vacuo. The residue was purified by column chromatography (petroleum ether/ethyl acetate=3/1 to 1/1) to afford N-(4-methoxybenzyl)-5-oxotetrahydrofuran-2-carboxamide (59 g, 236.70 mmol, 45% yield) as a yellow solid. ¹H NMR (400 MHz, DMSO-d₆) δ: 8.68 (br t, J=5.6 Hz, 1H) 7.19 (d, J=8.4 Hz, 2H) 6.87-6.91 (m, 2H) 4.89 (dd, J=8.0, 5.6 Hz, 1H) 4.23 (d, J=6.0 Hz, 2H) 3.73 (s, 3H) 2.51-2.55 (m, 1H) 2.36-2.47 (m, 1H) 2.01-2.16 (m, 1H).

### Step 6:

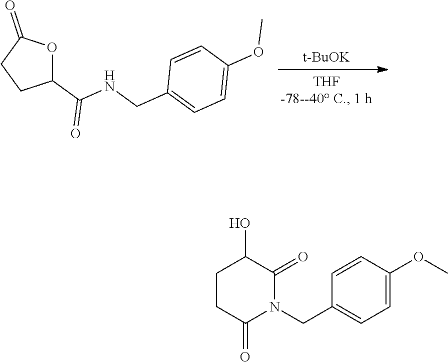 [SMILES: "t-BuOK" ; COc1ccc(CNC(=O)C2CCC(=O)O2)cc1 ; COc1ccc(CN2C(=O)CCC(O)C2=O)cc1]

To a solution of N-(4-methoxybenzyl)-5-oxotetrahydrofuran-2-carboxamide (10 g, 40.12 mmol, 1 eq) in tetrahydrofuran (120 mL) was added dropwise potassium 2-methylpropan-2-olate (1 M, 40.52 mL, 1.01 eq) at −78° C. under nitrogen. The resulting reaction mixture was stirred at −40° C. for 1 h. The reaction mixture was quenched by the addition of an aqueous solution of ammonium chloride (50 mL) at −40° C., and then diluted with water (150 mL) and extracted with ethyl acetate (100 mL×3). The combined organic layers were washed with brine (100 mL), dried over anhydrous sodium sulfate, filtered and concentrated under reduced pressure to give a residue. The residue was purified by silica gel column chromatography (petroleum ether/ethyl acetate=5/1 to 2/1) to afford 3-hydroxy-1-[(4-methoxyphenyl)methyl]piperidine-2,6-dione (7.5 g, 30.09 mmol, 75% yield) as a light yellow solid. ¹H NMR (400 MHz, CDCl₃) δ: 7.31 (m, 2H) 6.79-6.84 (m, 2H) 4.87 (s, 2H) 4.19 (ddd, J=12.4, 5.6, 1.2 Hz, 1H) 3.77 (s, 3H) 3.69 (d, J=1.6 Hz, 1H) 2.84-2.93 (m, 1H) 2.63 (m, 1H) 2.30 (m, 1H) 1.80-1.95 (m, 1H).

### Step 7:

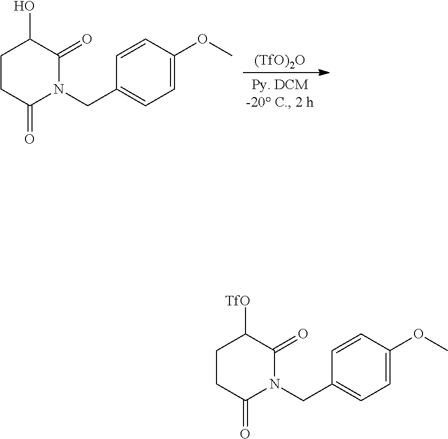 [SMILES: "(TfO)2O" ; COc1ccc(CN2C(=O)CCC(O)C2=O)cc1 ; COc1ccc(CN2C(=O)CCC(OS(=O)(=O)C(F)(F)F)C2=O)cc1]

To a solution of 3-hydroxy-1-(4-methoxybenzyl)piperidine-2,6-dione (3 g, 12.04 mmol, 1 eq) and pyridine (1.90 g, 24.02 mmol, 1.94 mL, 2.00 eq) in dichloromethane (100 mL) was added trifluoromethanesulfonic anhydride (5.67 g, 18.05 mmol, 1.5 eq) at −20° C. The mixture was stirred at −20° C. for 2 h. The reaction mixture was concentrated and purified by silica gel column chromatography (petroleum ether/ethyl acetate=10/1 to 2/1) to afford 1-(4-methoxybenzyl)-2,6-dioxopiperidin-3-yl trifluoromethanesulfonate (4.2 g, 11.01 mmol, 92% yield) as a colorless oil. ¹H NMR (400 MHz, CDCl₃) δ=7.39-7.33 (m, 2H), 6.86-6.81 (m, 2H), 5.33-5.27 (m, 1H), 4.91 (s, 2H), 3.79 (s, 3H), 3.04-2.95 (m, 1H), 2.80-2.68 (m, 1H), 2.46-2.26 (m, 2H)

### Step 8:

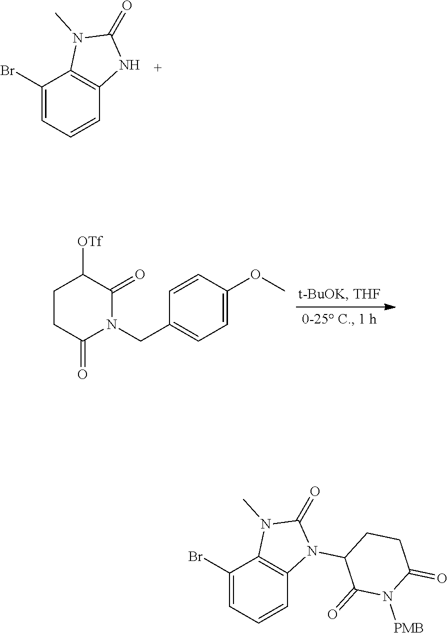

To a solution of 7-bromo-1-methyl-1H-benzo[d]imidazol-2(3H)-one (2.00 g, 8.81 mmol, 1 eq) in tetrahydrofuran (40 mL) was added potassium 2-methylpropan-2-olate (1 M, 10.67 mL, 1.21 eq). The mixture was stirred at 0° C. for 0.5 h prior to the dropwise addition of 1-(4-methoxybenzyl)-2,6-dioxopiperidin-3-yl trifluoromethanesulfonate (4.2 g, 11.01 mmol, 1.25 eq) in tetrahydrofuran (20 mL). The mixture was stirred at 0-25° C. for 0.5 h. The reaction mixture was quenched by the addition 10% ammonium chloride solution (10 mL) at 0° C. The reaction stirred at 0° C. for 1 h and was then extracted with ethyl acetate (50 mL×3). The combined organic layers were washed with brine (50 mL), dried over anhydrous sodium sulfate, and concentrated under reduced pressure to give a solid. The residue was purified by silica gel column chromatography (petroleum ether/ethyl acetate=6/1 to 1/1) to afford 3-(4-bromo-3-methyl-2-oxo-2,3-dihydro-1H-benzo[d]imidazol-1-yl)-1-(4-methoxybenzyl)piperidine-2,6-dione (3.5 g, 7.64 mmol, 87% yield) as a white solid. MS (ESI) m/z: 460.1 [M+H]⁺. ¹H NMR (400 MHz, DMSO-d₆) δ=7.24 (dd, J=0.8, 8.0 Hz, 1H), 7.22-7.18 (m, 2H), 7.11-7.04 (br m, 1H), 6.97-6.92 (m, 1H), 6.88-6.82 (m, 2H), 5.57 (dd, J=5.6, 12.8 Hz, 1H), 4.79 (q, J=14.4 Hz, 2H), 3.72 (s, 3H), 3.64 (s, 3H), 3.12-2.97 (m, 1H), 2.89-2.64 (m, 2H), 2.13-2.01 (m, 1H).

### Step 9:

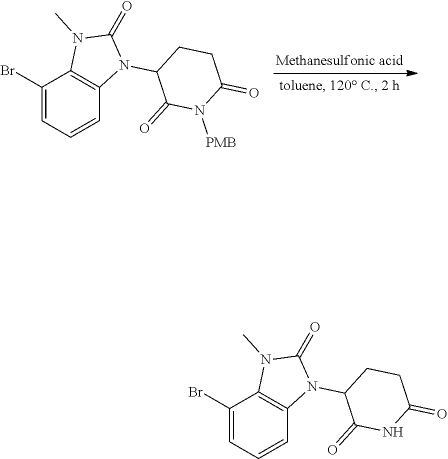

To a solution of 3-(4-bromo-3-methyl-2-oxo-2,3-dihydro-1H-benzo[d]imidazol-1-yl)-1-(4-methoxybenzyl)piperidine-2,6-dione (3.50 g, 7.64 mmol, 1 eq) in toluene (20 mL) was added methanesulfonic acid (15.75 g, 163.89 mmol, 11.67 mL, 21.46 eq) and the mixture was stirred at 120° C. for 2 h under an atmosphere of nitrogen. The reaction mixture was concentrated under reduced pressure to remove toluene. The residue was poured into water/ice (50 mL) and extracted with ethyl acetate (150 mL×3). The combined organic layers were washed with brine (50 mL), dried over anhydrous sodium sulfate, and concentrated under reduced pressure to give a residue. The residue was purified by silica gel column chromatography (petroleum ether/ethyl acetate=6/1 to 0/1) to afford 3-(4-bromo-3-methyl-2-oxo-2,3-dihydro-1H-benzo[d]imidazol-1-yl)piperidine-2,6-dione (1.8 g, 5.32 mmol, 70% yield) as a yellow solid. MS (ESI) m/z: 340.0 [M+H]⁺. ¹H NMR (400 MHz, DMSO-d₆) δ=11.13 (s, 1H), 7.28-7.22 (m, 1H), 7.17 (d, J=8.0 Hz, 1H), 7.01-6.95 (m, 1H), 5.41 (dd, J=5.2, 12.8 Hz, 1H), 3.63 (s, 3H), 2.95-2.82 (m, 1H), 2.77-2.59 (m, 2H), 2.08-1.98 (m, 1H).

### Step 10:

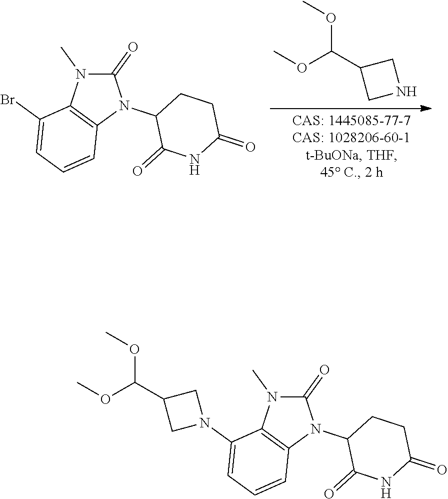

A reaction vessel charged with 3-(dimethoxymethyl)azetidine (150.0 mg, 1.14 mmol, 1.29 eq), 3-(4-bromo-3-methyl-2-oxo-2,3-dihydro-1H-benzo[d]imidazol-1-yl)piperidine-2,6-dione (300 mg, 887.16 μmol, 1 eq), [2-(2-aminophenyl)phenyl]-methylsulfonyloxy-palladium;dicyclohexyl-[2-(2,6-diisopropoxyphenyl)phenyl]phosphane (75.0 mg, 89.67 μmol, 0.101 eq), [2-(2-aminoethyl)phenyl]-chloro-palladium;dicyclohexyl-[2-(2,6-diisopropoxyphenyl)phenyl]phosphane;2-methoxy-2-methyl-propane (75.0 mg, 91.82 μmol, 1.03e-1 eq) and sodium 2-methylpropan-2-olate (1 M, 1.80 mL, 2.03 eq) in tetrahydrofuran (15 mL) was de-gassed and then heated to 45° C. for 2 h under a N₂ atmosphere. The reaction mixture was concentrated under reduced pressure to give a residue. The residue was purified by preparative HPLC (column: Shim-pack C18 150×25×10 m; mobile phase: [water (0.225% formic acid)-acetonitrile]; B %: 25%-45%, 10 min) to yield 3-(4-(3-(dimethoxymethyl)azetidin-1-yl)-3-methyl-2-oxo-2,3-dihydro-1H-benzo[d]imidazol-1-yl)piperidine-2,6-dione (20 mg, 51.49 μmol, 9% yield) as a white solid. MS (ESI) m/z: 389.2 [M+H]⁺. ¹H NMR (400 MHz, CDCl₃) δ=8.04-7.97 (m, 1H), 7.03-6.94 (m, 1H), 6.70 (d, J=7.6 Hz, 1H), 6.46 (d, J=8.0 Hz, 1H), 5.24-5.14 (m, 1H), 4.68 (d, J=7.2 Hz, 1H), 4.08-4.02 (m, 1H), 3.93 (t, J=8.4 Hz, 2H), 3.76-3.71 (m, 5H), 3.39 (s, 6H), 3.00-2.91 (m, 2H), 2.87-2.66 (m, 2H), 2.30-2.16 (m, 1H).

### Step 11:

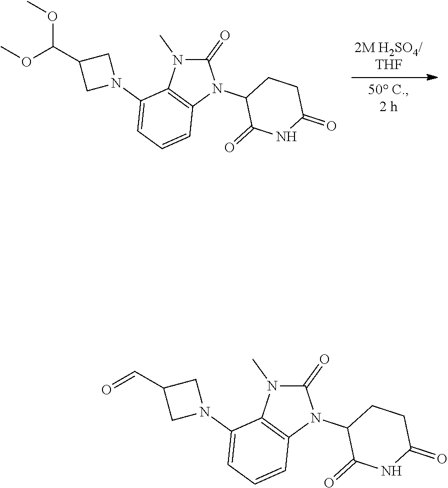 [SMILES: COC(OC)C1CN(c2cccc3c2n(C)c(=O)n3C2CCC(=O)NC2=O)C1 ; Cn1c(=O)n(C2CCC(=O)NC2=O)c2cccc(N3CC(C=O)C3)c21]

To a mixture of 3-(4-(3-(dimethoxymethyl)azetidin-1-yl)-3-methyl-2-oxo-2,3-dihydro-1H-benzo[d]imidazol-1-yl)piperidine-2,6-dione (8 mg, 20.6 μmol, 1 eq) in tetrahydrofuran (0.4 mL) was added sulphuric acid (2 M, 400.0 uL, 38.84 eq). The reaction was allowed to stir at 50° C. for 2 h under a N₂ atmosphere. The reaction mixture was quenched by addition of an aqueous 10% sodium bicarbonate solution (2 mL) at 0° C., and then extracted with DCM (4 mL×10). The combined organic layers were washed with brine (10 mL), dried over anhydrous sodium sulfate, filtered, and concentrated under reduced pressure to afford 1-(1-(2,6-dioxopiperidin-3-yl)-3-methyl-2-oxo-2,3-dihydro-1H-benzo[d]imidazol-4-yl)azetidine-3-carbaldehyde (7 mg, 20.45 μmol, 99% yield) as a yellow solid which was used into the next step without further purification. MS (ESI) m/z: 361.2 [M+H₂O]⁺.

### Step 12:

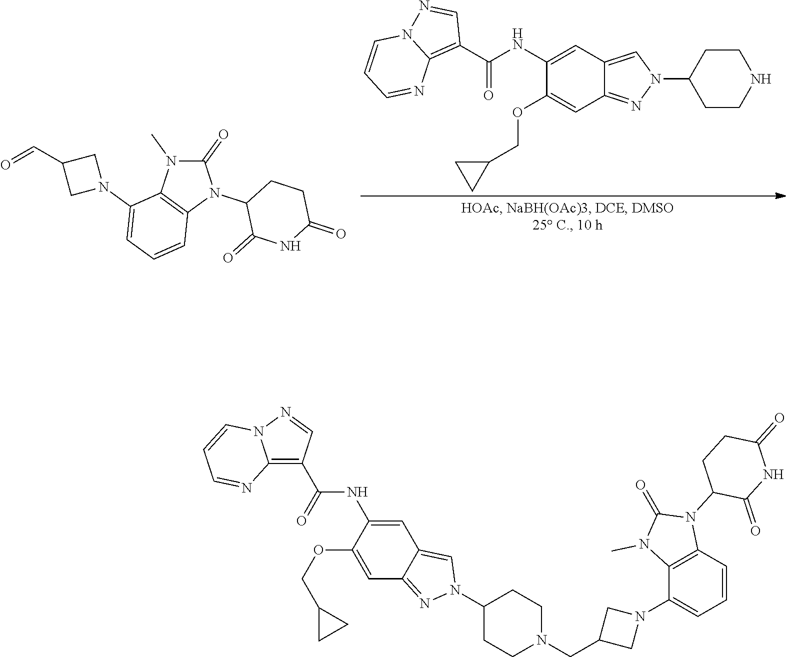 [SMILES: Cn1c(=O)n(C2CCC(=O)NC2=O)c2cccc(N3CC(C=O)C3)c21 ; O=C(Nc1cc2cn(C3CCNCC3)nc2cc1OCC1CC1)c1cnn2cccnc12 ; Cn1c(=O)n(C2CCC(=O)NC2=O)c2cccc(N3CC(CN4CCC(n5cc6cc(NC(=O)c7cnn8cccnc78)c(OCC7CC7)cc6n5)CC4)C3)c21]

To a solution of 1-(1-(2,6-dioxopiperidin-3-yl)-3-methyl-2-oxo-2,3-dihydro-1H-benzo[d]imidazol-4-yl)azetidine-3-carbaldehyde (125 mg, 365.13 μmol, 1 eq), N-(6-(cyclopropylmethoxy)-1-oxo-2-(piperidin-4-yl)isoindolin-5-yl)pyrazolo[1,5-a]pyrimidine-3-carboxamide (150 mg, 347.63 μmol, 9.52e-1 eq) in dimethyl sulfoxide (5 mL) and 1,2-dichloroethane (5 mL) was added acetic acid (63.0 mg, 1.05 mmol, 60 μL, 2.87 eq). The mixture was stirred at 25° C. for 30 min and then sodium triacetoxyborohydride (350 mg, 1.65 mmol, 4.52 eq) was added to the mixture. The mixture was stirred at 25° C. for 9.5 h. The reaction mixture was quenched by the addition of water (25 mL) and treated with a saturated sodium bicarbonate solution until the pH was measured as 8. The mixture was extracted with dichloromethane (100 mL×3), and the combined organic phase was washed with brine (100 mL), dried over anhydrous sodium sulfate, filtered, and concentrated under reduced pressure to give a residue. The residue was purified by preparative thin layer chromatography (silicon dioxide, dichloromethane/methanol=12:1) to give the desired compound, which was further purified by preparative HPLC (column: Unisil 3-100 C18 Ultra 150×50 mm×3 m; mobile phase: [water (0.225% formic acid)-acetonitrile]; B %: 18%-38%, 10 min). Compound N-(6-(cyclopropylmethoxy)-2-(1-((1-(1-(2,6-dioxopiperidin-3-yl)-3-methyl-2-oxo-2,3-dihydro-1H-benzo[d]imidazol-4-yl)azetidin-3-yl)methyl)piperidin-4-yl)-2H-indazol-5-yl)pyrazolo[1,5-a]pyrimidine-3-carboxamide (15.9 mg, 18.59 μmol, 500 yield, 94% purity, formic acid) was obtained as a white solid. MS (ESI) m/z: 758.2 [M+H]⁺. ¹H NMR (400 MHz, DMSO-d₆) δ=11.08 (s, 1H), 10.65 (s, 1H), 9.38 (dd, J=1.6, 7.2 Hz, 1H), 8.87 (dd, J=1.6, 4.4 Hz, 1H), 8.74 (s, 1H), 8.71 (s, 1H), 8.33 (s, 1H), 8.31 (s, 1H), 7.32 (dd, J=4.0, 6.8 Hz, 1H), 7.04 (s, 1H), 6.96 (t, J=8.0 Hz, 1H, 1H), 6.73 (d, J=8.0 Hz, 1H), 6.66 (d, J=8.0 Hz, 1H), 5.36-5.29 (m, 1H), 4.41-4.31 (in, 1H)), 4.02 (d, J=7.2 Hz, 2H), 3.96 (br t, J 6.8 Hz, 2H), 3.58 (s, 3H), 3.54-3.51 (m, 2H), 3.01-2.95 (m, 2H), 2.93-2.84 (m, 2H), 2.69-2.58 (m, 4H), 2.22-2.13 (m, 2H), 2.11-1.97 (m, 5H), 1.56-1.45 (m, 1H), 0.75-0.67 (m, 2H), 0.48-0.43 (m, 2H).

TABLE 8
The following compounds may be prepared in an analogous fashion as described for the
synthesis of Example 378.

| | | ¹H NMR |
|---|---|---|
| | | All spectra acquired at 400 MHz unless |
| Example | MS | otherwise indicated |
| Example 378 | See Above | |
| 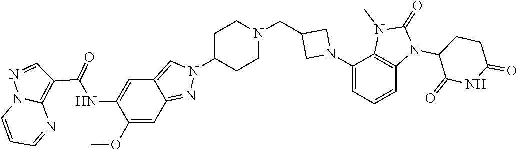 [SMILES: COc1cc2nn(C3CCN(CC4CN(c5cccc6c5n(C)c(=O)n6C5CCC(=O)NC5=O)C4)CC3)cc2cc1NC(=O)c1cnn2cccnc12] | 718.4 | CDCl3: δ = 10.47 (s, 1H), 8.85 (s, 1H), 8.83 (dd, J = 1.6, 7.2 Hz, 1H), 8.81-8.78 (m, 1H), 8.76 (dd, J = 1.6, 4.0 Hz, 1H), 8.05 (br s, 1H), 7.88 (s, 1H), 7.10-6.98 (m, 3H), 6.69 (d, J = 8.0 Hz, 1H), 6.46 (d, J = 8.0 Hz, 1H), 5.24-5.15 (m, 1H), 4.44- 4.33 (m, 1H), 4.06 (s, 3H), 4.04-3.98 (m, 2H), 3.79-3.67 (m, 6H), 3.60 (t, J = 6.4 Hz, 2H), 3.15-3.04 (m, 2H), 2.99-2.90 (m, 2H), |
| Example 379 | | 2.88-2.71 (m, 4H), 2.32-2.18 (m, 6H) |
| 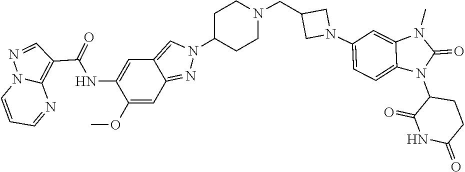 [SMILES: COc1cc2nn(C3CCN(CC4CN(c5ccc6c(c5)n(C)c(=O)n6C5CCC(=O)NC5=O)C4)CC3)cc2cc1NC(=O)c1cnn2cccnc12] | 718.2 | DMSO-d₆ δ = 11.08-11.01 (m, 1H), 10.54 (s, 1H), 9.38 (dd, J = 1.6, 6.8 Hz, 1H), 8.96 (dd, J = 1.6, 4.4 Hz, 1H), 8.71 (s, 1H), 8.68 (s, 1H), 8.34 (s, 1H), 8.32 (s, 1H), 7.38-7.33 (m, 1H), 7.11 (s, 1H), 6.90 (d, J = 8.4 Hz, 1H), 6.32 (d, J = 2.0 Hz, 1H), 6.11 (dd, J = 2.0, 8.8 Hz, 1H), 5.31-5.23 (m, 1H), 4.42-4.33 (m, 1H), 4.03 (s, 3H), 3.94 (t, J = 7.2 Hz, 2H), 3.46- 3.43 (m, 2H), 3.28 (s, 3H), 3.00-2.83 (m, 4H), 2.64 (d, J = 7.6 Hz, 2H), 2.60- |
| Example 380 | | 2.55 (m, 1H), 2.24-1.91 (m, 8H) |
| 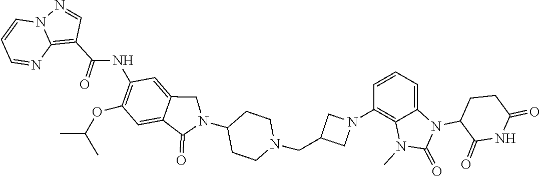 [SMILES: CC(C)Oc1cc2c(cc1NC(=O)c1cnn3cccnc13)CN(C1CCN(CC3CN(c4cccc5c4n(C)c(=O)n5C4CCC(=O)NC4=O)C3)CC1)C2=O] | 761.3 | DMSO-d₆ δ = 11.08 (s, 1H), 10.76 (s, 1H), 9.41 (dd, J = 1.6, 6.8 Hz, 1H), 8.90 (dd, J = 2.0, 4.4 Hz, 1H), 8.74 (s, 2H), 8.27 (s, 1H), 7.36 (dd, J = 4.0, 6.8 Hz, 1H), 7.34 (s, 1H), 6.96 (s, 1H), 6.73 (d, J = 8.0 Hz, 1H), 6.66 (d, J = 8.0 Hz, 1H), 5.36-5.28 (m, 2H), 4.91 (d, J = 5.6 Hz, 1H), 4.43 (s, 2H), 4.00-3.93 (m, 3H), 3.58 (s, 3H), 3.51 (br s, 2H), 2.99-2.83 (m, 4H), 2.62 (br d, J = 7.2 Hz, 1H), 2.12-2.06 (m, 2H), 2.04-1.95 (m, 4H), 1.83-1.75 (m, 2H), |
| Example 381 | | 1.73-1.66 (m, 2H), 1.44 (d, J = 6.0 Hz, 6H) |
| 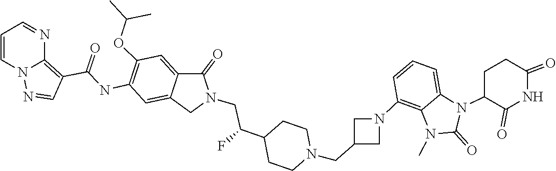 | 807.2 | DMSO-d₆ δ = 11.07 (s, 1H), 10.76 (s, 1H), 9.41 (dd, J = 1.6, 7.2 Hz, 1H), 8.90 (dd, J = 1.6, 4.0 Hz, 1H), 8.74 (s, 2H), 7.42- 7.32 (m, 2H), 6.94 (t, J = 8.0 Hz, 1H), 6.72 (br d, J = 7.6 Hz, 1H), 6.64 (br d, J = 8.0 Hz, 1H), 5.35-5.28 (m, 1H), 4.98-4.85 (m, 1H), 4.66-4.45 (m, 3H), 3.98-3.90 (m, 2H), 3.86-3.69 (m, 2H), 3.56 (s, 3H), 3.51-3.46 (m, 2H), 2.93-2.81 (m, 4H), 2.03-1.88 (m, 4H), 1.82-1.76 (m, 1H), 1.74-1.68 (m, 1H), 1.63-1.49 (m, 2H), |
| Example 382 | | 1.45 (d, J = 6.0 Hz, 6H), 1.40-1.29 (m, 4H) |
| 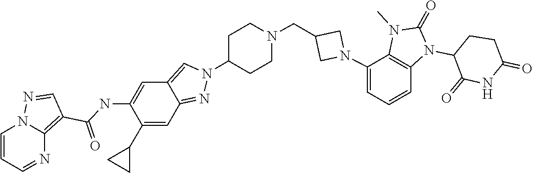 | 728.2 | DMSO-d₆ δ = 11.09 (br s, 1H), 10.35 (s, 1H), 9.39 (dd, J = 1.6, 6.8 Hz, 1H), 8.91 (dd, J = 1.6, 4.4 Hz, 1H), 8.73 (s, 1H), 8.64 (s, 1H), 8.38 (s, 1H), 8.32 (s, 1H), 7.42 (s, 1H), 7.33 (dd, J = 4.4, 6.8 Hz, 1H), 6.96 (1, J = 8.0 Hz, 1H), 6.73 (br d, J = 7.6 Hz, 1H), 6.66 (d, J = 8.8 Hz, 1H), 5.36-5.29 (m, 1H), 4.45-4.39 (m, 1H), 3.96 (t, J = 6.8 Hz, 2H), 3.58 (s, 3H), 3.53 (1, J = 6.0 Hz, 2H), 2.98 (br d, J = 10.8 Hz, 2H), 2.92-2.84 (m, 2H), 2.67-2.59 |
| Example 383 | | (m, 4H), 2.23-2.15 (m, 2H), 2.12-2.05 |
| | | (m, 5H), 2.01-1.95 (m, 1H), 1.20-1.15 |
| | | (m, 2H), 0.80-0.75 (m, 2H) |

### Preparation of Example 384: N-(2-((1r,4r)-4-((1-(1-(2,6-dioxopiperidin-3-yl)-3-methyl-2-oxo-2,3-dihydro-1H-benzo[d]imidazol-4-yl)azetidin-3-yl)(methyl)amino)cyclohexyl)-6-isopropoxy-1-oxoisoindolin-5-yl)pyrazolo[1,5-a]pyrimidine-3-carboxamide

### Step 1:

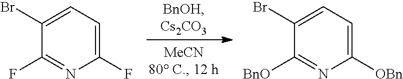 [SMILES: Fc1ccc(Br)c(F)n1 ; Brc1ccc(OCc2ccccc2)nc1OCc1ccccc1]

To a solution of 3-bromo-2,6-difluoropyridine (10 g, 51.55 mmol, 1 eq) and benzyl alcohol (13.99 g, 129.40 mmol, 13.45 mL, 2.51 eq) in acetonitrile (100 mL) was added cesium carbonate (50.39 g, 154.66 mmol, 3 eq). The reaction mixture was stirred at 80° C. for 12 h. The reaction mixture was filtered, and the filtrate was concentrated under reduced pressure to give a residue. The residue was purified by silica gel column chromatography (eluted with petroleum ether/ethyl acetate=100/1 to 10/1) to afford 2,6-bis(benzyloxy)-3-bromopyridine (18.4 g, 49.7 mmol, 96% yield) as a white solid. MS (ESI) m/z: 370.0 [M+H]⁺. ¹H NMR (400 MHz, CDCl₃) δ: 7.68 (d, J=8.4 Hz, 1H) 7.31-7.48 (m, 10H) 6.33 (d, J=8.4 Hz, 1H) 5.44 (s, 2H) 5.31 (s, 2H).

### Step 2:

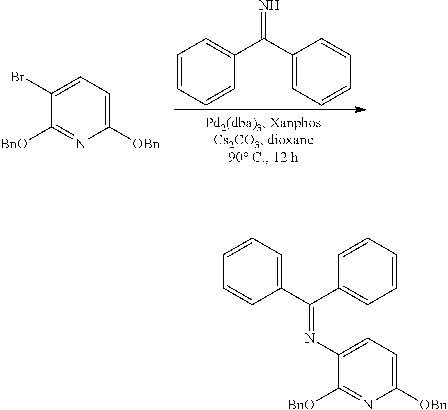 [SMILES: Brc1ccc(OCc2ccccc2)nc1OCc1ccccc1 ; N=C(c1ccccc1)c1ccccc1 ; c1ccc(COc2ccc(N=C(c3ccccc3)c3ccccc3)c(OCc3ccccc3)n2)cc1]

To a mixture of 2,6-bis(benzyloxy)-3-bromopyridine (10 g, 27.01 mmol, 1 eq), diphenylmethanimine (14.69 g, 81.03 mmol, 13.60 mL, 3 eq) and cesium carbonate (26.67 g, 81.85 mmol, 3.03 eq) in dioxane (100 mL) was added Xantphos (1.67 g, 2.88 mmol, 0.107 eq) and Pd2(dba)₃ (2.50 g, 2.73 mmol, 0.101 eq). The suspension was degassed and purged with nitrogen in 3 cycles. The mixture was stirred under nitrogen at 90° C. for 12 h. The reaction mixture was filtered and washed with tetrahydrofuran (30 mL×3). The collected filtrate was concentrated to give 2,6-bis(benzyloxy)-N-(diphenylmethylene)pyridin-3-amine (12.7 g, crude) was obtained as a yellow oil which was used for the next step without further purification. MS (ESI) m/z: 471.5 [M+H]⁺.

### Step 3:

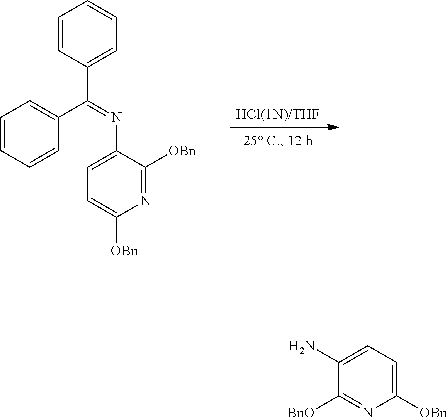 [SMILES: c1ccc(COc2ccc(N=C(c3ccccc3)c3ccccc3)c(OCc3ccccc3)n2)cc1 ; Nc1ccc(OCc2ccccc2)nc1OCc1ccccc1]

A solution of 2,6-bis(benzyloxy)-N-(diphenylmethylene)pyridin-3-amine (12.7 g, 26.99 mmol, 1 eq, crude) in tetrahydrofuran (100 mL) and hydrochloric acid (1 M, 100 mL, 3.71 eq) was stirred at 25° C. for 12 h. The pH of the reaction mixture was adjusted to pH=8 with the addition of a saturated sodium bicarbonate solution at 0° C. The reaction mixture was extracted with ethyl acetate (100 mL×3) and the combined organic layers were washed with brine (80 mL×2), dried over anhydrous sodium sulfate, filtered and concentrated to give a residue. The residue was purified by silica gel column chromatography (petroleum ether/ethyl acetate=3/1 to 1/1) to afford 2,6-bis(benzyloxy)pyridin-3-amine (8 g, 26.11 mmol, 96% yield) as a brown oil. MS (ESI) m/z: 307.1 [M+H]⁺. ¹H NMR (400 MHz, CDCl₃) δ: 7.30-7.46 (m, 10H) 6.98 (d, J=8.0 Hz, 1H) 6.29 (d, J=8.0 Hz, 1H) 5.40 (s, 2H) 5.29 (s, 2H) 3.44 (br s, 2H).

### Step 4:

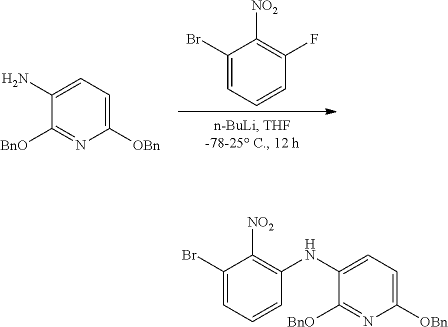 [SMILES: Nc1ccc(OCc2ccccc2)nc1OCc1ccccc1 ; O=[N+]([O-])c1c(F)cccc1Br ; O=[N+]([O-])c1c(Br)cccc1Nc1ccc(OCc2ccccc2)nc1OCc1ccccc1]

To a solution of 2,6-bis(benzyloxy)pyridin-3-amine (7 g, 22.85 mmol, 1 eq) in tetrahydrofuran (100 mL) was dropwise added n-butyllithium (2.5 M, 11.88 mL, 1.3 eq) at −78° C. The reaction mixture was stirred at −78° C. for 1 h prior to the dropwise addition of 1-bromo-3-fluoro-2-nitro-benzene (5.60 g, 25.46 mmol, 1.11 eq) in tetrahydrofuran (20 mL) at −78° C. The mixture was then stirred at 25° C. for 11 h. The mixture was poured into ice-water (150 mL). The aqueous phase was extracted with ethyl acetate (100 mL×3). The combined organic phase was washed with brine (100 mL), dried with anhydrous sodium sulfate, filtered, and concentrated under vacuum. The residue was purified by silica gel column chromatography (petroleum ether/ethyl acetate=20/1 to 10/1) to afford 2,6-bis(benzyloxy)-N-(3-bromo-2-nitrophenyl)pyridin-3-amine (8.5 g, 16.79 mmol, 73% yield) as a red oil. MS (ESI) m/z: 508.0 [M+H]⁺.

### Step 5: —

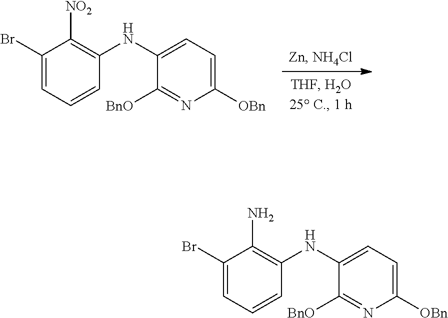 [SMILES: O=[N+]([O-])c1c(Br)cccc1Nc1ccc(OCc2ccccc2)nc1OCc1ccccc1 ; Nc1c(Br)cccc1Nc1ccc(OCc2ccccc2)nc1OCc1ccccc1]

To a solution of 2,6-bis(benzyloxy)-N-(3-bromo-2-nitrophenyl)pyridin-3-amine (8.5 g, 16.79 mmol, 1 eq) in tetrahydrofuran (80 mL) and water (80 mL) was added zinc (9 g, 137.64 mmol, 8.20 eq) and ammonium chloride (9 g, 168.25 mmol, 10.02 eq). The reaction mixture was stirred at 25° C. for 1 h. The mixture was filtered to remove the zinc, and then the pH of the filtrate was adjusted with the addition of a saturated aqueous solution of sodium bicarbonate until it measured at 8. The mixture was extracted with ethyl acetate (100 mL×3). The combined organic layers were washed with brine (80 mL×2), dried over anhydrous sodium sulfate, filtered, and concentrated in vacuo to give a residue. The residue was purified by silica gel column chromatography (petroleum ether/ethyl acetate=10/1) to afford N¹-(2,6-bis(benzyloxy)pyridin-3-yl)-3-bromobenzene-1,2-diamine (8 g, crude) as a red oil. MS (ESI) m/z: 476.2 [M+H]⁺.

### Step 6:

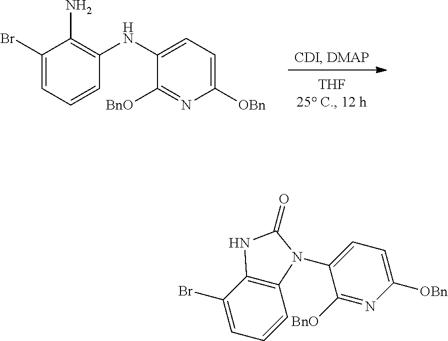 [SMILES: Nc1c(Br)cccc1Nc1ccc(OCc2ccccc2)nc1OCc1ccccc1 ; O=c1[nH]c2c(Br)cccc2n1-c1ccc(OCc2ccccc2)nc1OCc1ccccc1]

To a solution of N¹-(2,6-bis(benzyloxy)pyridin-3-yl)-3-bromobenzene-1,2-diamine (8 g, 16.79 mmol, 1 eq, crude) in tetrahydrofuran (10 mL) was added 1,1-carbonyldimidazole (6.81 g, 41.98 mmol, 2.5 eq) and stirred at 25° C. for 0.5 h. Dimethylaminopyridine (205.17 mg, 1.68 mmol, 0.1 eq) was added stirring continued at 25° C. for 11.5 h. The mixture was concentrated under reduced pressure to give a residue. The residue was purified by silica gel column chromatography (petroleum ether/ethyl acetate=5/1 to 3/1) to afford 1-(2,6-bis(benzyloxy)pyridin-3-yl)-4-bromo-1H-benzo[d]imidazol-2(3H)-one (5.5 g, 9.96 mmol, 59% yield) as a white solid. MS (ESI) m/z: 504.0 [M+H]⁺.

### Step 7:

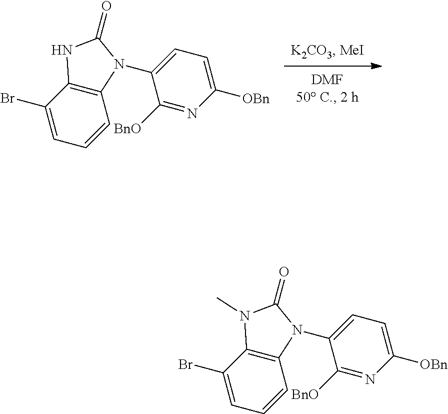 [SMILES: O=c1[nH]c2c(Br)cccc2n1-c1ccc(OCc2ccccc2)nc1OCc1ccccc1 ; Cn1c(=O)n(-c2ccc(OCc3ccccc3)nc2OCc2ccccc2)c2cccc(Br)c21]

To a solution of 1-(2,6-bis(benzyloxy)pyridin-3-yl)-4-bromo-1H-benzo[d]imidazol-2(3H)-one (5.5 g, 10.95 mmol, 1 eq) and potassium carbonate (3.03 g, 21.90 mmol, 2 eq) in N,N-dimethylformamide (50 mL) was added iodomethane (9.12 g, 64.25 mmol, 4 mL, 5.87 eq). The reaction mixture was allowed to stir at 50° C. for 2 h. The mixture was filtered, and the filtrate was concentrated in vacuo to give a residue. The residue was purified by silica gel column chromatography (petroleum ether/ethyl acetate=10/1 to 3/1) to afford 1-(2,6-bis(benzyloxy)pyridin-3-yl)-4-bromo-3-methyl-1H-benzo[d]imidazol-2(3H)-one (4.8 g, 9.30 mmol, 84% yield) as a light brown solid. MS (ESI) m/z: 518.1 [M+H]⁺. ¹H NMR (400 MHz, DMSO-d₆) δ: 7.82 (d, J=8.0 Hz, 1H) 7.34-7.46 (m, 5H) 7.23-7.28 (m, 6H) 6.92 (t, J=8.0 Hz, 1H) 6.68 (dd, J=7.6, 0.80 Hz, 1H) 6.62 (d, J=8.0 Hz, 1H) 5.33-5.43 (m, 4H) 3.67 (s, 3H).

### Step 8:

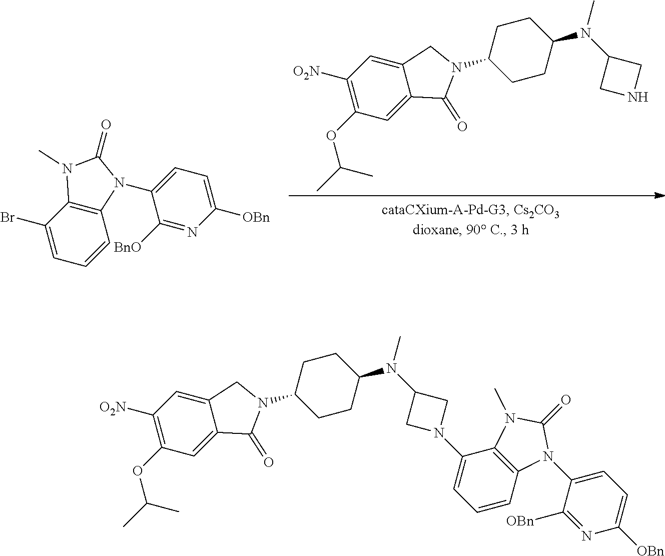 [SMILES: Cn1c(=O)n(-c2ccc(OCc3ccccc3)nc2OCc2ccccc2)c2cccc(Br)c21 ; CC(C)Oc1cc2c(cc1[N+](=O)[O-])CN([C@H]1CC[C@H](N(C)C3CNC3)CC1)C2=O ; CC(C)Oc1cc2c(cc1[N+](=O)[O-])CN([C@H]1CC[C@H](N(C)C3CN(c4cccc5c4n(C)c(=O)n5-c4ccc(OCc5ccccc5)nc4OCc4ccccc4)C3)CC1)C2=O]

A reaction vessel charged with 1-(2,6-bis(benzyloxy)pyridin-3-yl)-4-bromo-3-methyl-1H-benzo[d]imidazol-2(3H)-one (577.3 mg, 1.12 mmol, 1 eq), 2-((1r,4r)-4-(azetidin-3-ylamino)cyclohexyl)-6-isopropoxy-5-nitroisoindolin-1-one (0.45 g, 1.12 mmol, 1 eq), cataCXium A Pd G3 (81.42 mg, 111.81 μmol, 0.1 eq) and cesium carbonate (910.7 mg, 2.80 mmol, 2.5 eq) in dioxane (8 mL) was de-gassed and then heated to 90° C. for 3 h under a nitrogen atmosphere. The reaction mixture was filtered and washed with dichloromethane (10 mL×2). The collected filtrate was concentrated to give a residue. The residue was purified by preparative thin-layer chromatography (dichloromethane/methanol=20/1) to afford 1-(2,6-bis(benzyloxy)pyridin-3-yl)-4-(3-(((1r,4r)-4-(6-isopropoxy-5-nitro-1-oxoisoindolin-2-yl)cyclohexyl)(methyl)amino)azetidin-1-yl)-3-methyl-1H-benzo[d]imidazol-2(3H)-one (0.42 g, 471.14 μmol, 42% yield) as a brown solid. MS (ESI) m/z: 838.2 [M+H]⁺.

### Step 9:

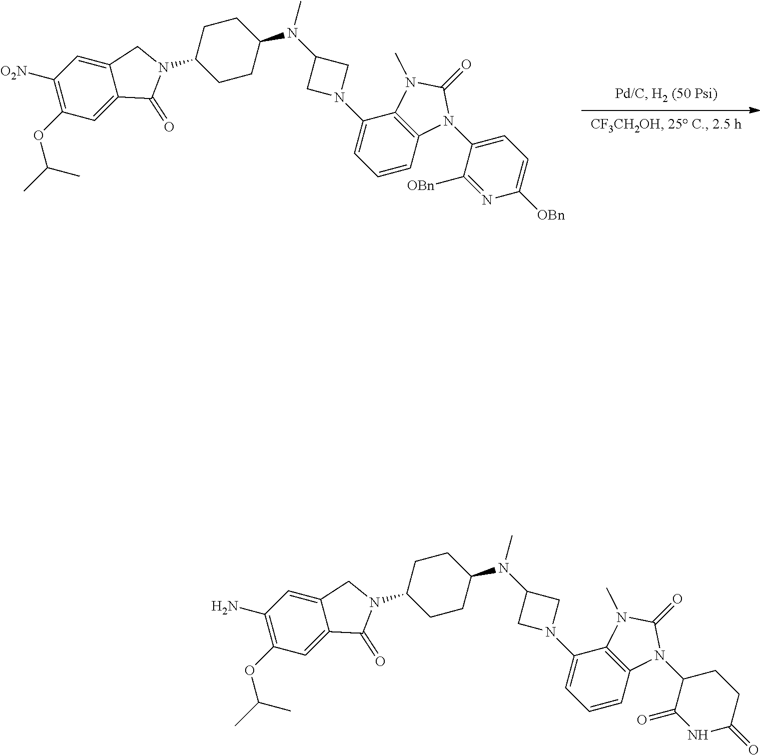 [SMILES: CC(C)Oc1cc2c(cc1[N+](=O)[O-])CN([C@H]1CC[C@H](N(C)C3CN(c4cccc5c4n(C)c(=O)n5-c4ccc(OCc5ccccc5)nc4OCc4ccccc4)C3)CC1)C2=O ; CC(C)Oc1cc2c(cc1N)CN([C@H]1CC[C@H](N(C)C3CN(c4cccc5c4n(C)c(=O)n5C4CCC(=O)NC4=O)C3)CC1)C2=O]

To a solution of 1-(2,6-bis(benzyloxy)pyridin-3-yl)-4-(3-(((1r,4r)-4-(6-isopropoxy-5-nitro-1-oxoisoindolin-2-yl)cyclohexyl)(methyl)amino)azetidin-1-yl)-3-methyl-1H-benzo[d]imidazol-2(3H)-one (50 mg, 59.67 μmol, 1 eq) in trifluoroethanol (10 mL) was added palladium on carbon (10%, 50 mg) under a nitrogen atmosphere. The suspension was degassed and purged with hydrogen in 3 cycles. The mixture was stirred under hydrogen (50 psi) at 25° C. for 2.5 h. The reaction mixture was filtered and washed with dichloromethane/methyl alcohol (5/1, 20 mL×2). The collected filtrate was concentrated to give a residue. The residue was purified by preparative thin-layer chromatography (dichloromethane/methyl alcohol=10/1) to afford 3-(4-(3-(((1r,4r)-4-(5-amino-6-isopropoxy-1-oxoisoindolin-2-yl)cyclohexyl)(methyl)amino)azetidin-1-yl)-3-methyl-2-oxo-2,3-dihydro-1H-benzo[d]imidazol-1-yl)piperidine-2,6-dione (25 mg, 39.70 mol, 66% yield) as a brown solid. MS (ESI) m/z: 630.4 [M+H]⁺.

### Step 10:

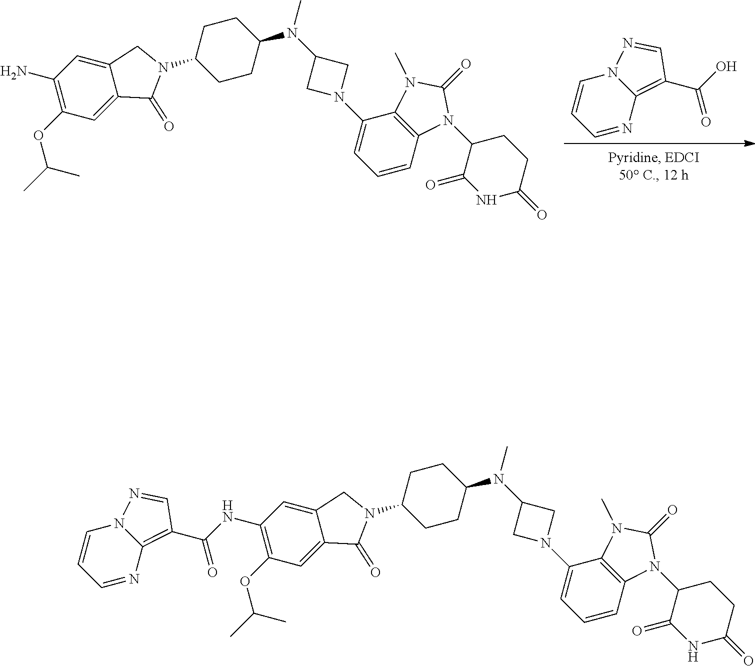 [SMILES: CC(C)Oc1cc2c(cc1N)CN([C@H]1CC[C@H](N(C)C3CN(c4cccc5c4n(C)c(=O)n5C4CCC(=O)NC4=O)C3)CC1)C2=O ; O=C(O)c1cnn2cccnc12 ; CC(C)Oc1cc2c(cc1NC(=O)c1cnn3cccnc13)CN([C@H]1CC[C@H](N(C)C3CN(c4cccc5c4n(C)c(=O)n5C4CCC(=O)NC4=O)C3)CC1)C2=O]

To a solution of pyrazolo[1,5-a]pyrimidine-3-carboxylic acid (35.0 mg, 214.53 μmol, 1.93 eq) in pyridine (5 mL) was added 3-(4-(3-(((1r,4r)-4-(5-amino-6-isopropoxy-1-oxoisoindolin-2-yl)cyclohexyl)(methyl)amino)azetidin-1-yl)-3-methyl-2-oxo-2,3-dihydro-1H-benzo[d]imidazol-1-yl)piperidine-2,6-dione (70 mg, 111.16 μmol, 1 eq) and 1-(3-dimethylaminopropyl)-3-ethylcarbodiimide hydrochloride (105.05 mg, 548.0 μmol, 4.93 eq). The mixture was stirred at 50° C. for 12 h. The reaction mixture was concentrated to give a residue. The residue was purified by preparative thin-layer chromatography (dichloromethane/methanol=10/1) to give the desired product which was further purified by semi-preparative reverse phase HPLC (column: Phenomenex luna C18 150×25 mm×10 um; mobile phase: [water (0.225% formic acid)-acetonitrile]; B %: 14%-44%, 10 min). N-(2-((1r,4r)-4-((1-(1-(2,6-dioxopiperidin-3-yl)-3-methyl-2-oxo-2,3-dihydro-1H-benzo[d]imidazol-4-yl)azetidin-3-yl)(methyl)amino)cyclohexyl)-6-isopropoxy-1-oxoisoindolin-5-yl)pyrazolo[1,5-a]pyrimidine-3-carboxamide (40.4 mg, 51.62 mol, 46% yield, 99% purity) was obtained as an off-white solid. MS (ESI) m/z: 775.3 [M+H]⁺. ¹H NMR (DMSO-d₆, 400 MHz) δ:11.08 (s, 1H) 10.75 (s, 1H) 9.40 (d, J=7.2 Hz, 1H) 8.89 (d, J=3.6 Hz, 1H) 8.73 (s, 2H) 7.32-7.39 (m, 2H) 6.96 (t, J=8.0 Hz, 1H) 6.68-6.78 (m, 2H) 5.32 (dd, J=12.8, 4.8 Hz, 1H) 4.85-4.95 (m, 1H) 4.38 (s, 2H) 3.98 (br s, 1H) 3.91 (br s, 2H) 3.61-3.71 (m, 3H) 3.59 (s, 3H) 2.83-2.94 (m, 1H) 2.59-2.73 (m, 2H) 2.45 (br s, 1H) 2.16 (s, 3H) 2.00 (m, 1H) 1.78 (br s, 4H) 1.58-1.67 (m, 2H) 1.49 (br s, 2H) 1.44 (d, J=6.0 Hz, 6H).

TABLE 9
The following compounds may be prepared in an analogous fashion as described for the
synthesis of Example 384.

| | | ¹H NMR |
|---|---|---|
| | | All spectra acquired at 400 MHz unless |
| Example | MS | otherwise indicated |
| Example 384 | See Above | |
| 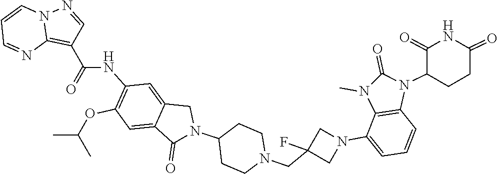 [SMILES: CC(C)Oc1cc2c(cc1NC(=O)c1cnn3cccnc13)CN(C1CCN(CC3(F)CN(c4cccc5c4n(C)c(=O)n5C4CCC(=O)NC4=O)C3)CC1)C2=O] | 779.3 | DMSO-d₆ δ = 11.22-10.91 (m, 1H), 10.77-10.73 (m, 1H), 9.43-9.37 (m, 1H), 8.91-8.87 (m, 1H), 8.76-8.71 (m, 2H), 7.38-7.32 (m, 2H), 7.03-6.96 (m, 1H), 6.83-6.76 (m, 2H), 5.39-5.30 (m, 1H), 4.93-4.85 (m, 1H), 4.47-4.38 (m, 2H), 4.06-3.88 (m, 5H), 3.61-3.57 (m, 3H), 3.08-3.00 (m, 2H), 2.85 (br d, J = 5.4 Hz, 3H), 2.68-2.62 (m, 1H), 2.39-2.29 (m, 3H), 2.04-1.96 (m, 1H), 1.91-1.78 (m, 2H), 1.74-1.65 (m, 2H), 1.48-1.40 (m, 6H) |
| Example 385 | | |
| 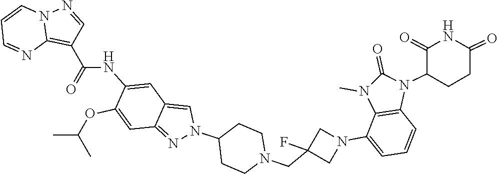 [SMILES: CC(C)Oc1cc2nn(C3CCN(CC4(F)CN(c5cccc6c5n(C)c(=O)n6C5CCC(=O)NC5=O)C4)CC3)cc2cc1NC(=O)c1cnn2cccnc12] | 764.2 | DMSO-d₆ δ = 11.10 (s, 1 H), 10.59 (s, 1 H), 9.37 (dd, J = 1.6, 6.8 Hz, 1 H), 8.86 (dd, J = 1.2, 4.0 Hz, 1 H), 8.75 (s, 1 H), 8.70 (s, 1 H),8.31(s, 1H),7.33 (dd, J = 4.4, 7.2 Hz, 1 H), 7.13 (s, 1 H), 7.02 - 6.96 (m, 1 H), 6.79 (m, 2 H), 5.34 (m, 1 H), 4.85 (m, 1 H), 4.43-4.32 (m, 1 H), 4.04-3.91 (m, 4 H), 3.59 (s, 3 H), 3.06 (m, 2 H), 2.99 (s, 1 H), 2.92 (s, 1 H), 2.91- 2.84 (m, 1 H), 2.72-2.58 (m, 2 H), 2.45- 2.37 (m, 2 H), 2.16-2.04 (m, 4 H), 2.03- |
| Example 386 | | 1.96 (m, 1 H), 1.47 (d, J = 6.0 Hz, 6 H) |
| 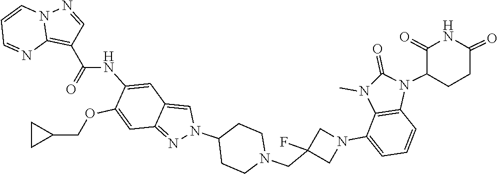 [SMILES: Cn1c(=O)n(C2CCC(=O)NC2=O)c2cccc(N3CC(F)(CN4CCC(n5cc6cc(NC(=O)c7cnn8cccnc78)c(OCC7CC7)cc6n5)CC4)C3)c21] | 776.2 | DMSO-d₆ δ = 11.10 (s, 1H), 10.66 (s, 1H), 9.39 (dd, J = 1.6, 7.2 Hz, 1H), 8.88 (dd, J = 1.6, 4.4 Hz, 1H), 8.74 (d, J = 11.2 Hz, 2H), 8.32 (s, 1H), 7.33 (dd,J = 4.4, 6.8 Hz, 1H), 7.04 (s, 1H), 7.03-6.97 (m, 1H), 6.85-6.77 (m, 2H), 5.35 (br dd, J = 5.2, 12.4 Hz, 1H), 4.44-4.33 (m, 1H), 4.05-3.91 (m, 6H), 3.60 (s, 3H), 3.08 (br d, J = 11.2 Hz, 2H), 3.03-2.93 (m, 2H), 2.92-2.84 (m, 1H), 2.75-2.60 (m, 2H), 2.43 (br t, J = 10.4 Hz, 2H), 2.19-2.05 |
| Example 387 | | (m, 4H), 2.03-1.97 (m, 1H), 1.56-1.45 |
| | | (m, 1H), 0.75-0.68 (m, 2H), 0.46 (q, J = |
| | | 4.8 Hz, 2H). |
| 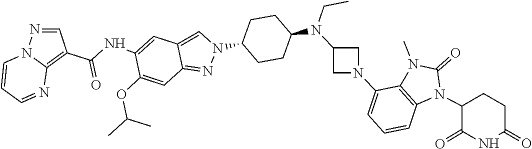 [SMILES: CCN(C1CN(c2cccc3c2n(C)c(=O)n3C2CCC(=O)NC2=O)C1)[C@H]1CC[C@H](n2cc3cc(NC(=O)c4cnn5cccnc45)c(OC(C)C)cc3n2)CC1] | 792.3 | DMSO-d₆ δ = 11.09 (br s, 1 H), 10.59 (s, 1 H), 9.38 (dd, J= 1.6,7.2 Hz, 1H), 8.87 (dd, J = 1.6, 4.0 Hz, 1 H), 8.74 (s, 1 H), 8.70 (s, 1 H), 8.34 (s, 1 H), 8.24 (s, 1 H), 7.33 (dd, J = 4.4, 7.2 Hz, 1 H), 7.12 (s, 1 H), 6.97 (1, J = 8.0 Hz, 1 H), 6.73 (br dd, J = 7.6, 19.2 Hz, 2 H), 5.33 (br dd, J = 5.2, 12.4 Hz, 1 H), 4.89-4.80 (m, 1 H), 4.41-4.31 (m, 1H), 3.98-3.90 (m, 2 H), |
| Example 388 | | 3.89-3.83 (m, 1 H), 3.65-3.61 (m, 2 H), |
| | | 3.59 (s, 3 H), 2.95-2.83 (m, 1 H), 2.71- |
| | | 2.58 (m, 5 H), 2.15 (br d, J = 10.0 Hz, 2 |
| | | H), 2.03-1.90 (m, 3 H), 1.87-1.79 (m, |
| | | 2 H), 1.59-1.49 (m, 2 H), 1.47 (d, J = |
| | | 5.6 Hz, 6 H), 1.01 (br t, J = 7.2 Hz, 3 H) |
| 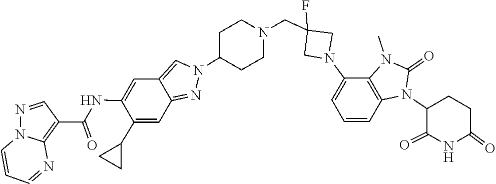 [SMILES: C=Nc1c(C(=O)Nc2cc3cn(C4CCN(CC5(F)CN(c6cccc7c6n(C)c(=O)n7C6CCC(=O)NC6=O)C5)CC4)nc3cc2C2CC2)cnn1/C=C\C] | 746.3 | DMSO-d₆ δ = 11.09 (s, 1 H), 10.34 (s, 1 H), 9.38 (dd, J = 7.2, 1.6 Hz, 1 H), 8.91 (dd, J = 4.0, 1.6 Hz, 1 H), 8.73 (s, 1 H), 8.64 (s, 1 H), 8.38 (s, 1 H), 7.42 (s, 1 H), 7.29-7.36 (m, 1 H), 6.94-7.03 (m, 1 H), 6.74-6.84 (m, 2 H), 5.34 (dd, J = 12.8, 5.2 Hz, 1 H), 4.38-4.49 (m, 1 H), 3.90- 4.05 (m, 4 H), 3.59 (s, 3 H), 3.07 (d, J = 11.6 Hz, 2 H), 2.93-3.02 (m, 2 H), 2.82- 2.90 (m, 1 H), 2.58-2.71 (m, 2 H), 2.37- 2.47 (m, 2 H), 1.95-2.19 (m, 6 H), 1.12- 1.21 (m, 2 H), 0.73-0.82 (m, 2 H). |
| Example 389 | | |

### Preparation of Example 390: N-(6-(cyclopropylmethoxy)-2-(1-((1-(1-(2,6-dioxopiperidin-3-yl)-3-methyl-2-oxo-2,3-dihydro-1H-benzo[d]imidazol-4-yl)piperidin-4-yl)methyl)piperidin-4-yl)-2H-indazol-5-yl)pyrazolo [1,5-a]pyrimidine-3-carboxamide

### Step 1:

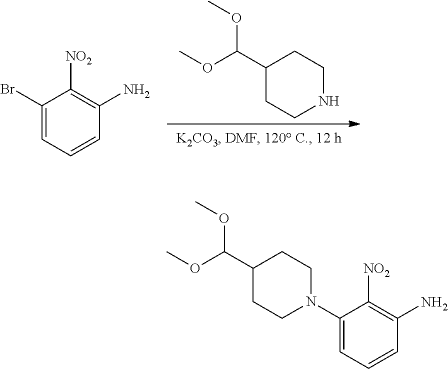 [SMILES: Nc1cccc(Br)c1[N+](=O)[O-] ; COC(OC)C1CCNCC1 ; COC(OC)C1CCN(c2cccc(N)c2[N+](=O)[O-])CC1]

A reaction vessel charged with 4-(dimethoxymethyl)piperidine (3 g, 18.84 mmol, 2.04 eq), 3-bromo-2-nitroaniline (2 g, 9.22 mmol, 1 eq), potassium carbonate (4.00 g, 28.94 mmol, 3.14 eq) and DMF (20 mL) was de-gassed and then heated to 120° C. for 12 h under an atmosphere of nitrogen. The reaction mixture was quenched by the addition of water (20 mL) at 0° C., and then extracted with ethyl acetate (50 mL×3). The combined organic layers were washed with brine (50 mL), dried over anhydrous sodium sulfate, filtered, and concentrated in vacuo to give a residue. The residue was purified by column chromatography (silicon dioxide eluted with petroleum ether:dichloromethane=2/1 to 0/1) to afford 3-(4-(dimethoxymethyl)piperidin-1-yl)-2-nitroaniline (1.7 g, 5.76 mmol, 62% yield) as a yellow solid. MS (ESI) m/z: 296.1 [M+H]⁺. ¹H NMR (400 MHz, CDCl₃) δ=7.10 (t, J=8.0 Hz, 1H), 6.44-6.31 (m, 2H), 4.79 (br s, 2H), 4.08 (d, J=7.2 Hz, 1H), 3.37 (s, 6H), 3.26 (br d, J=12.4 Hz, 2H), 2.71 (dt, J=1.6, 12.0 Hz, 2H), 1.82-1.67 (m, 3H), 1.52-1.39 (m, 2H).

### Step 2:

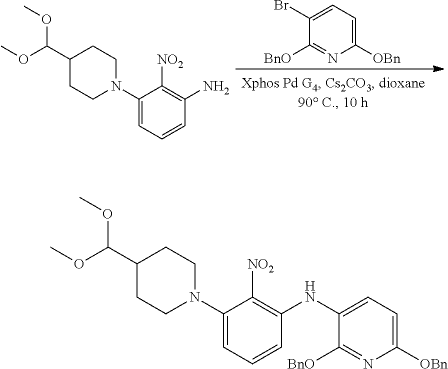 [SMILES: COC(OC)C1CCN(c2cccc(N)c2[N+](=O)[O-])CC1 ; Brc1ccc(OCc2ccccc2)nc1OCc1ccccc1 ; COC(OC)C1CCN(c2cccc(Nc3ccc(OCc4ccccc4)nc3OCc3ccccc3)c2[N+](=O)[O-])CC1]

A mixture of 3-(4-(dimethoxymethyl)piperidin-1-yl)-2-nitroaniline (1.20 g, 4.06 mmol, 1.5 eq), 2,6-bis(benzyloxy)-3-bromopyridine (1 g, 2.70 mmol, 1 eq), cesium carbonate (2.65 g, 8.13 mmol, 203.16 uL, 3.01 eq), Xphos Pd G4 (CAS: 1599466-81-5, 250.0 mg, 290.54 μmol, 0.108 eq) in dioxane (10 mL) was degassed and purged with a nitrogen atmosphere in 3 cycles. The mixture was then stirred at 90° C. for 10 h under an atmosphere of nitrogen. The reaction mixture was quenched by addition of water (50 mL) at 0° C., and then extracted with ethyl acetate (100 mL×3). The combined organic layers were washed with brine (100 mL), dried over anhydrous sodium sulfate, filtered and concentrated under reduced pressure to give a residue. The residue was purified by column chromatography (silicon dioxide eluted with petroleum ether:dichloromethane=2/1 to 0/1) to afford 2,6-bis(benzyloxy)-N-(3-(4-(dimethoxymethyl)piperidin-1-yl)-2-nitrophenyl)pyridin-3-amine (1.2 g, 2.05 mmol, 76% yield) obtained as a red solid. MS (ESI) m/z: 585.2 [M+H]⁺. ¹H NMR (400 MHz, CDCl₃) δ=7.49-7.29 (m, 10H), 7.19 (s, 1H), 7.12 (t, J=8.4 Hz, 1H), 6.58 (d, J=8.4 Hz, 1H), 6.50 (d, J=8.4 Hz, 1H), 6.38 (d, J=8.4 Hz, 1H), 5.40 (s, 2H), 5.32 (s, 2H), 4.09 (d, J=7.2 Hz, 1H), 3.72 (s, 1H), 3.38 (s, 6H), 3.30 (br d, J=12.0 Hz, 2H), 2.80-2.70 (m, 2H), 1.84-1.67 (m, 3H), 1.53-1.41 (m, 2H).

### Step 3:

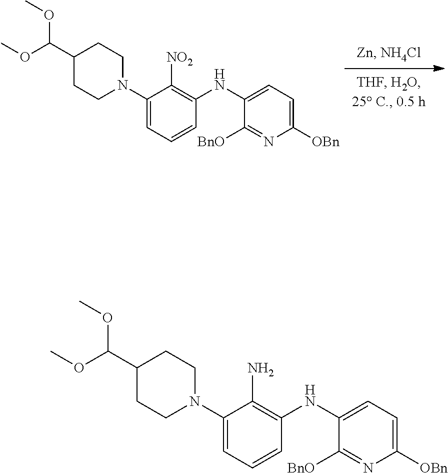 [SMILES: COC(OC)C1CCN(c2cccc(Nc3ccc(OCc4ccccc4)nc3OCc3ccccc3)c2[N+](=O)[O-])CC1 ; COC(OC)C1CCN(c2cccc(Nc3ccc(OCc4ccccc4)nc3OCc3ccccc3)c2N)CC1]

To a solution of 2,6-bis(benzyloxy)-N-(3-(4-(dimethoxymethyl)piperidin-1-yl)-2-nitrophenyl)pyridin-3-amine (1.2 g, 2.05 mmol, 1 eq) in tetrahydrofuran (60 mL) was added zinc (1.20 g, 18.35 mmol, 8.94 eq) and ammonium chloride (1.20 g, 22.43 mmol, 10.93 eq) in water (60 mL). The mixture was stirred at 25° C. for 0.5 h. The mixture was filtered to remove the zinc, and the filtrate was diluted with a saturated sodium bicarbonate solution (50 mL). The mixture was extracted with ethyl acetate (150 mL×3). The combined organic layers were washed with brine (150 mL), dried over anhydrous sodium sulfate, filtered and concentrated in vacuo to give N1-(2,6-bis(benzyloxy)pyridin-3-yl)-3-(4-(dimethoxymethyl)piperidin-1-yl)benzene-1,2-diamine (1.1 g, 1.98 mmol, 97% yield) as a yellow gum which was used in the next step without further purification. MS (ESI) m/z: 555.3 [M+H]⁺.

### Step 4:

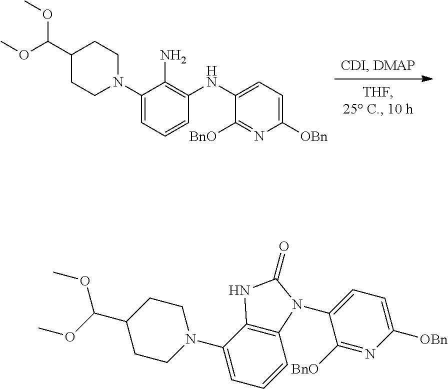 [SMILES: COC(OC)C1CCN(c2cccc(Nc3ccc(OCc4ccccc4)nc3OCc3ccccc3)c2N)CC1 ; COC(OC)C1CCN(c2cccc3c2[nH]c(=O)n3-c2ccc(OCc3ccccc3)nc2OCc2ccccc2)CC1]

To a solution of N1-(2,6-bis(benzyloxy)pyridin-3-yl)-3-(4-(dimethoxymethyl)piperidin-1-yl)benzene-1,2-diamine (1.1 g, 1.98 mmol, 1 eq) in tetrahydrofuran (30 mL) was added dimethylaminopyridine (50 mg, 409.27 μmol, 0.206 eq) and di(1H-imidazol-1-yl)methanone (900 mg, 5.55 mmol, 2.80 eq) at 0° C. The mixture was stirred at 25° C. for 10 h. The mixture was concentrated under reduced pressure to give a residue. The residue was purified by column chromatography (silicon dioxide eluted with petroleum ether/ethyl acetate=4/1 to 1/1) to afford 1-(2,6-bis(benzyloxy)pyridin-3-yl)-4-(4-(dimethoxymethyl)piperidin-1-yl)-1H-benzo[d]imidazol-2(3H)-one (1.1 g, 1.89 mmol, 96% yield) as a yellow solid. MS (ESI) m/z: 581.3 [M+H]⁺. ¹H NMR (400 MHz, CDCl₃) δ=9.69 (br s, 1H), 7.66 (d, J=8.0 Hz, 1H), 7.47-7.32 (m, 5H), 7.28-7.19 (m, 5H), 6.95 (t, J=8.0 Hz, 1H), 6.74 (br d, J=7.6 Hz, 1H), 6.54 (d, J=8.4 Hz, 1H), 6.43 (br d, J=7.6 Hz, 1H), 5.49-5.32 (m, 4H), 4.02 (br d, J=7.2 Hz, 1H), 3.46-3.38 (m, 2H), 3.35 (s, 6H), 2.68 (br t, J=10.0 Hz, 2H), 1.83 (br d, J=12.8 Hz, 2H), 1.77-1.70 (m, 1H), 1.61-1.45 (m, 2H).

### Step 5:

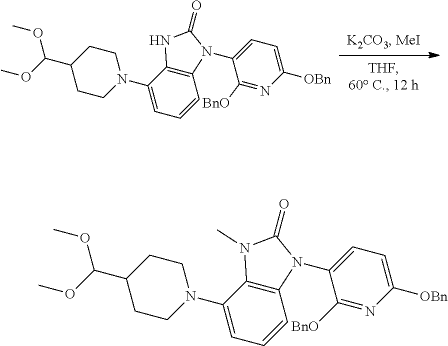 [SMILES: COC(OC)C1CCN(c2cccc3c2[nH]c(=O)n3-c2ccc(OCc3ccccc3)nc2OCc2ccccc2)CC1 ; COC(OC)C1CCN(c2cccc3c2n(C)c(=O)n3-c2ccc(OCc3ccccc3)nc2OCc2ccccc2)CC1]

To a solution of 1-(2,6-bis(benzyloxy)pyridin-3-yl)-4-(4-(dimethoxymethyl)piperidin-1-yl)-1H-benzo[d]imidazol-2(3H)-one (0.55 g, 947.18 μmol, 1 eq) in tetrahydrofuran (15 mL) was added potassium carbonate (275 mg, 1.99 mmol, 2.10 eq) and methyl iodide (1.34 g, 9.47 mmol, 589.3 μL, 10 eq) at 0° C. The mixture was stirred at 60° C. for 12 h. The mixture was concentrated in vacuum to give a residue. The residue was purified by column chromatography (silicon dioxide eluted with petroleum ether/ethyl acetate=6/1 to 1/1) to afford 1-(2,6-bis(benzyloxy)pyridin-3-yl)-4-(4-(dimethoxymethyl)piperidin-1-yl)-3-methyl-1H-benzo[d]imidazol-2(3H)-one (560 mg, 941.65 μmol, 99% yield) as a yellow solid. MS (ESI) m/z: 595.2 [M+H]⁺. ¹H NMR (400 MHz, CDCl₃) δ=7.59 (d, J=8.4 Hz, 1H), 7.46-7.32 (m, 5H), 7.27-7.22 (m, 5H), 6.97-6.88 (m, 2H), 6.51 (d, J=8.4 Hz, 1H), 6.46 (dd, J=2.0, 6.8 Hz, 1H), 5.49-5.42 (m, 1H), 5.36-5.27 (m, 3H), 4.14 (t, J=7.2 Hz, 1H), 3.81 (s, 3H), 3.41 (s, 6H), 3.27 (br s, 2H), 2.75 (br t, J=11.2 Hz, 2H), 1.89 (br d, J=13.2 Hz, 2H), 1.85-1.73 (m, 1H), 1.59-1.53 (m, 2H).

### Step 6:

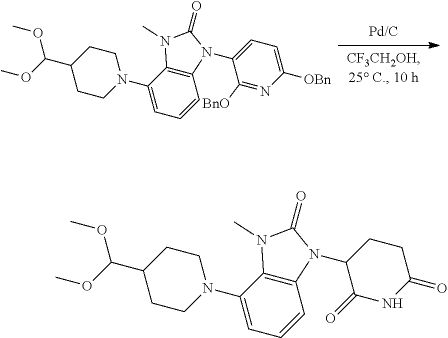 [SMILES: COC(OC)C1CCN(c2cccc3c2n(C)c(=O)n3-c2ccc(OCc3ccccc3)nc2OCc2ccccc2)CC1 ; COC(OC)C1CCN(c2cccc3c2n(C)c(=O)n3C2CCC(=O)NC2=O)CC1]

To a solution of 1-(2,6-bis(benzyloxy)pyridin-3-yl)-4-(4-(dimethoxymethyl)piperidin-1-yl)-3-methyl-1H-benzo[d]imidazol-2(3H)-one (0.2 g, 336.3 μmol, 1 eq) in 2,2,2-trifluoroethanol (5 mL) was added palladium on carbon (200.0 mg, 187.93 μmol, 10% purity, 0.559 q) under a nitrogen atmosphere. The suspension was degassed under vacuum and purged with hydrogen atmosphere several times. The mixture was stirred under a hydrogen atmosphere (15 psi) at 25° C. for 10 h. The reaction mixture was filtered and concentrated under reduced pressure to afford 3-(4-(4-(dimethoxymethyl)piperidin-1-yl)-3-methyl-2-oxo-2,3-dihydro-1H-benzo[d]imidazol-1-yl)piperidine-2,6-dione (125 mg, 300.14 μmol, 89% yield) as a white solid which was used in the next step without further purification. MS (ESI) m/z: 773.2 [M+H]⁺. ¹H NMR (400 MHz, CDCl₃) δ=8.27 (s, 1H), 7.03-6.97 (m, 1H), 6.94-6.89 (m, 1H), 6.60-6.54 (m, 1H), 5.21 (dd, J=5.6, 12.8 Hz, 1H), 4.13 (br d, J=6.8 Hz, 1H), 3.79-3.75 (m, 3H), 3.40 (s, 6H), 3.21 (br d, J=10.8 Hz, 2H), 2.98-2.89 (m, 1H), 2.88-2.65 (m, 4H), 2.26-2.17 (m, 1H), 1.87 (br d, J=12.8 Hz, 2H), 1.80-1.75 (m, 1H), 1.60-1.46 (m, 2H).

### Step 7:

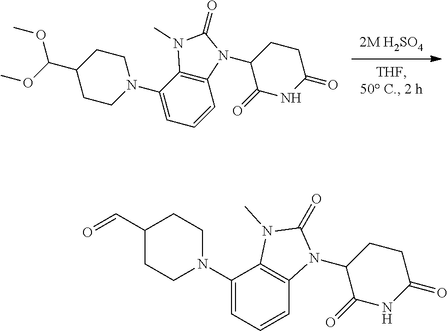 [SMILES: COC(OC)C1CCN(c2cccc3c2n(C)c(=O)n3C2CCC(=O)NC2=O)CC1 ; Cn1c(=O)n(C2CCC(=O)NC2=O)c2cccc(N3CCC(C=O)CC3)c21]

To a mixture of 3-[4-[4-(dimethoxymethyl)-1-piperidyl]-3-methyl-2-oxo-benzimidazol-1-yl]piperidine-2,6-dione (120 mg, 288.14 μmol, 1 eq) in tetrahydrofuran (4 mL) was added sulphuric acid (2 M, 3.73 mL, 25.89 eq) and the mixture was stirred at 50° C. for 2 h under a nitrogen atmosphere. The reaction mixture was diluted with water (20 mL), and the pH was adjusted to 8 with the addition of a saturated aqueous solution of sodium bicarbonate. The mixture was extracted with dichloromethane (4 mL×10), and the combined organic layers were washed with brine (10 mL×3), dried over anhydrous sodium sulfate, filtered and concentrated under reduced pressure to yield 1-(1-(2,6-dioxopiperidin-3-yl)-3-methyl-2-oxo-2,3-dihydro-1H-benzo[d]imidazol-4-yl)piperidine-4-carbaldehyde (106 mg, 286.18 μmol, 99% yield) as a yellow solid which was used in the next step without further purification. MS (ESI) m/z: 371.1 [M+H]⁺.

### Step 8:

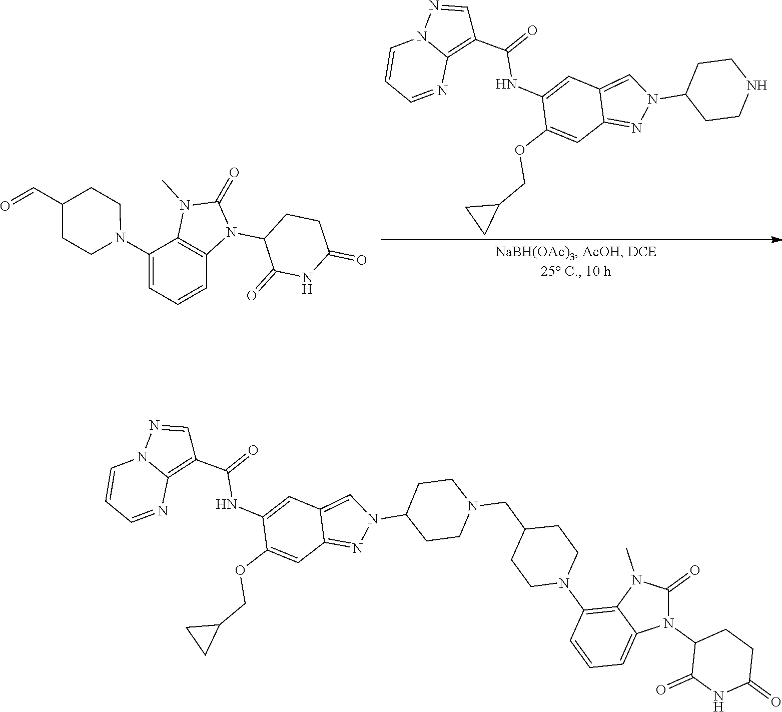 [SMILES: Cn1c(=O)n(C2CCC(=O)NC2=O)c2cccc(N3CCC(C=O)CC3)c21 ; O=C(Nc1cc2cn(C3CCNCC3)nc2cc1OCC1CC1)c1cnn2cccnc12 ; Cn1c(=O)n(C2CCC(=O)NC2=O)c2cccc(N3CCC(CN4CCC(n5cc6cc(NC(=O)c7cnn8cccnc78)c(OCC7CC7)cc6n5)CC4)CC3)c21]

To a solution of 1-(1-(2,6-dioxopiperidin-3-yl)-3-methyl-2-oxo-2,3-dihydro-1H-benzo[d]imidazol-4-yl)piperidine-4-carbaldehyde (106 mg, 286.2 μmol, 1 eq), N-(6-(cyclopropylmethoxy)-2-(piperidin-4-yl)-2H-indazol-5-yl)pyrazolo[1,5-a]pyrimidine-3-carboxamide (123.5 mg, 286.2 μmol, 1 eq) in 1,2-dichloroethane (3 mL) and dimethyl sulfoxide (3 mL) was added acetic acid (52.5 mg, 874.24 μmol, 50.0 μL, 3.05 eq). The mixture was stirred at 25° C. for 30 min followed by the addition of sodium triacetoxyborohydride (280 mg, 1.32 mmol, 4.62 eq). The mixture was stirred at 25° C. for 9.5 h. To the reaction mixture was added water (10 mL) and the mixture was extracted with ethyl acetate (15 mL×3). The combined organic phase was washed with brine (15 mL×3), dried over anhydrous sodium sulfate, filtered, and concentrated in vacuo. The residue was purified by preparative thin-layer chromatography (silicon dioxide, dichloromethane:methanol=10:1) to afford N-(6-(cyclopropylmethoxy)-2-(1-((1-(1-(2,6-dioxopiperidin-3-yl)-3-methyl-2-oxo-2,3-dihydro-1H-benzo[d]imidazol-4-yl)piperidin-4-yl)methyl)piperidin-4-yl)-2H-indazol-5-yl)pyrazolo[1,5-a]pyrimidine-3-carboxamide (82.5 mg, 101.8 μmol, 35% yield, 97% purity) as a yellow solid. MS (ESI) m/z: 786.3 [M+H]⁺. ¹H NMR (400 MHz, CDCl₃) δ=10.56 (s, 1H), 8.93-8.89 (m, 1H), 8.83 (dd, J=1.8, 7.0 Hz, 1H), 8.80 (s, 1H), 8.69 (dd, J=1.8, 4.0 Hz, 1H), 7.88 (s, 1H), 7.06-6.98 (m, 3H), 6.95-6.91 (m, 1H), 6.57 (d, J 7.8 Hz, 1H), 5.21 (dd, J=5.6, 12.4 Hz, 1H), 4.42-4.27 (m, 1H), 4.03 (d, J=6.8 Hz, 2H), 3.78 (s, 3H), 3.21 (br d, J=10.0 Hz, 2H), 3.07 (br d, J=5.8 Hz, 2H), 2.99-2.81 (m, 2H), 2.80-2.71 (m, 3H), 2.33 (br d, J=7.0 Hz, 2H), 2.29-2.14 (m, 8H), 1.97-1.87 (m, 3H), 1.44-1.39 (m, 2H), 1.09 (br d, J=3.2 Hz, 1H), 0.75-0.70 (m, 2H), 0.56-0.51 (m, 2H).

### Preparation of Example 391: 6-chloro-N-(2-(1-((1-(2-(2,6-dioxopiperidin-3-yl)-1,3-dioxoisoindolin-5-yl)azetidin-3-yl)methyl)piperidin-4-yl)-6-(2-hydroxypropan-2-yl)-2H-indazol-5-yl)pyrazolo[1,5-a]pyrimidine-3-carboxamide

### Step 1:

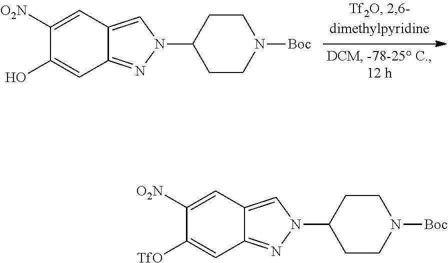 [SMILES: CC(C)(C)OC(=O)N1CCC(n2cc3cc([N+](=O)[O-])c(O)cc3n2)CC1 ; CC(C)(C)OC(=O)N1CCC(n2cc3cc([N+](=O)[O-])c(OS(=O)(=O)C(F)(F)F)cc3n2)CC1]

To a solution of tert-butyl 4-(6-hydroxy-5-nitro-indazol-2-yl)piperidine-1-carboxylate (1 g, 2.76 mmol, 1 eq) in dichloromethane (10 mL) was added 2,6-dimethylpyridine (1.18 g, 11.04 mmol, 1.29 mL, 4 eq) and trifluoromethylsulfonic anhydride (1.17 g, 4.14 mmol, 683 μL, 1.5 eq) at −78° C. The mixture was then allowed to stir at 25° C. for 12 h. The reaction mixture was quenched by the addition of water (20 mL) at 0° C., and then diluted with dichloromethane (30 mL) and extracted with dichloromethane (10 mL×3). The combined organic layers were washed with brine (10 mL×3), dried over anhydrous sodium sulfate, filtered, and concentrated under reduced pressure to give a residue. The residue was purified by column chromatography (silicon dioxide, eluted with a gradient of petroleum ether/ethyl acetate solution from 10/1 to 0/1) to afford tert-butyl 4-(5-nitro-6-(((trifluoromethyl)sulfonyl)oxy)-2H-indazol-2-yl)piperidine-1-carboxylate (1 g, 2.02 mmol, 73% yield) as a brown oil. MS (ESI) m/z: 439.1 [M−56]⁺.

### Step 2:

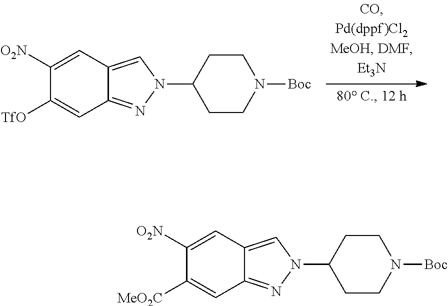 [SMILES: CC(C)(C)OC(=O)N1CCC(n2cc3cc([N+](=O)[O-])c(OS(=O)(=O)C(F)(F)F)cc3n2)CC1 ; COC(=O)c1cc2nn(C3CCN(C(=O)OC(C)(C)C)CC3)cc2cc1[N+](=O)[O-]]

To a solution of tert-butyl 4-(5-nitro-6-(((trifluoromethyl)sulfonyl)oxy)-2H-indazol-2-yl)piperidine-1-carboxylate (1 g, 2.02 mmol, 1 eq) in DMF (6 mL) and methanol (6 mL) was added triethylamine (1.02 g, 10.11 mmol, 1.41 mL, 5 eq) followed by [1,1′-bis(diphenylphosphino)ferrocene]dichloropalladium(II) (295.97 mg, 404.50 μmol, 0.2 eq). The mixture was degassed and purged with a nitrogen atmosphere in 3 cycles, and then the mixture was stirred at 80° C. for 12 h under an atmosphere of carbon monoxide (50 psi). The reaction mixture was quenched by the addition of water (10 mL) at 0° C., and then diluted with ethyl acetate (30 mL) and extracted with ethyl acetate (10 mL×3). The combined organic layers were washed with brine (10 mL×3), dried over anhydrous sodium sulfate, filtered, and concentrated under reduced pressure to give a residue. The residue was purified by column chromatography (silicon dioxide, petroleum ether/ethyl acetate=10/1 to 0/1) to afford methyl 5-amino-2-(1-(tert-butoxycarbonyl)piperidin-4-yl)-2H-indazole-6-carboxylate (300 mg, 801.21 μmol, 39% yield), obtained as a yellow oil. MS (ESI) m/z: 375.2 [M+H]⁺.

### Step 3:

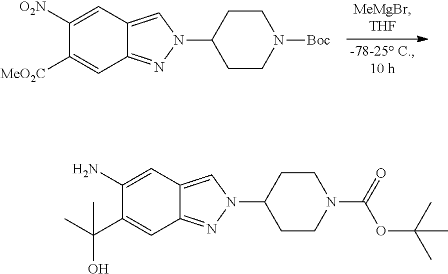 [SMILES: COC(=O)c1cc2nn(C3CCN(C(=O)OC(C)(C)C)CC3)cc2cc1[N+](=O)[O-] ; CC(C)(C)OC(=O)N1CCC(n2cc3cc(N)c(C(C)(C)O)cc3n2)CC1]

To a solution of methyl 5-amino-2-(1-tert-butoxycarbonyl-4-piperidyl)indazole-6-carboxylate (700 mg, 1.87 mmol, 1 eq) in tetrahydrofuran (7 mL) was added dropwise methylmagnesium bromide (3 M, 3.12 mL, 5 eq) at −78° C. under a nitrogen atmosphere. The resulting mixture was stirred at 25° C. for 12 h. The reaction mixture was quenched by the addition of a solution of ammonium chloride (10 mL) at 0° C., and then diluted with water (20 mL) and extracted with ethyl acetate (30 mL×2). The combined organic layers were washed with brine (10 mL×3), dried over anhydrous sodium sulfate, filtered, and concentrated under reduced pressure to give a residue. The residue was purified by column chromatography (silicon dioxide, petroleum ether/ethyl acetate=5/1 to 1/1) to afford tert-butyl 4-(5-amino-6-(2-hydroxypropan-2-yl)-2H-indazol-2-yl)piperidine-1-carboxylate (400 mg, 1.07 mmol, 57% yield) as a white solid. MS (ESI) m/z: 375.2 [M+H]⁺.

### Step 4:

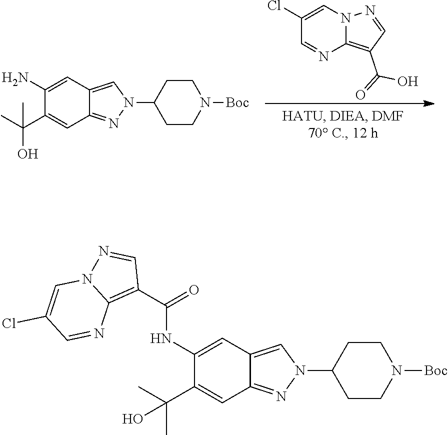 [SMILES: CC(C)(C)OC(=O)N1CCC(n2cc3cc(N)c(C(C)(C)O)cc3n2)CC1 ; O=C(O)c1cnn2cc(Cl)cnc12 ; CC(C)(C)OC(=O)N1CCC(n2cc3cc(NC(=O)c4cnn5cc(Cl)cnc45)c(C(C)(C)O)cc3n2)CC1]

To a solution of tert-butyl 4-(5-amino-6-(2-hydroxypropan-2-yl)-2H-indazol-2-yl)piperidine-1-carboxylate (400 mg, 1.07 mmol, 1 eq), 6-chloropyrazolo[1,5-a]pyrimidine-3-carboxylic acid (633.14 mg, 3.20 mmol, 3 eq) in N,N-dimethylformamide (10 mL) was added diisopropylethylamine (690.26 mg, 5.34 mmol, 930.3 μL, 5 eq) and HATU (1.22 g, 3.20 mmol, 3 eq). The mixture was stirred at 70° C. for 12 h. The reaction mixture was quenched by the addition of water (20 mL) at 0° C., and then diluted with ethyl acetate (30 mL) and extracted with ethyl acetate (10 mL×3). The combined organic layers were washed with brine (10 mL×3), dried over anhydrous sodium sulfate, filtered, and concentrated under reduced pressure to give a residue. The residue was purified by column chromatography (silicon dioxide, eluted with a gradient of ethyl acetate:methanol of 1/0 to 5/1) to afford tert-butyl 4-(5-(6-chloropyrazolo[1,5-a]pyrimidine-3-carboxamido)-6-(2-hydroxypropan-2-yl)-2H-indazol-2-yl)piperidine-1-carboxylate (480 mg, 866.36 μmol, 81% yield) as a yellow oil. MS (ESI) m/z: 536.2 [M−H₂O]⁺.

### Step 5:

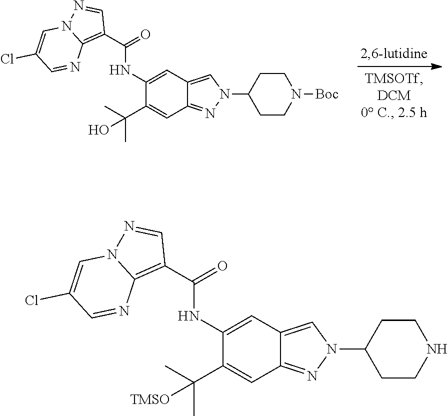

To stirred of tert-butyl 4-(5-(6-chloropyrazolo[1,5-a]pyrimidine-3-carboxamido)-6-(2-hydroxypropan-2-yl)-2H-indazol-2-yl)piperidine-1-carboxylate (480 mg, 866.4 μmol, 1 eq) in dichloromethane (10 mL) at 0° C., was added 2,6-dimethylpyridine (1.86 g, 17.33 mmol, 2.02 mL, eq) and trimethylsilyl trifluoromethanesulfonate (1.93 g, 8.66 mmol, 1.57 mL, 10 eq) dropwise. Then the mixture was stirred at 0° C. for 2 h, after which a solution of sodium bicarbonate (2 mL) was added, followed by the addition of water (10 mL) at 0° C. The mixture was diluted with dichloromethane (30 mL) and extracted with dichloromethane (10 mL×3). The combined organic layers were dried over anhydrous sodium sulfate, filtered, and concentrated under reduced pressure to give a residue. The residue was purified by preparative TLC (silicon dioxide, eluted with a 10:1 solution of petroleum ether:ethyl acetate) to afford 6-chloro-N-(2-(piperidin-4-yl)-6-(2-((trimethylsilyl)oxy)propan-2-yl)-2H-indazol-5-yl)pyrazolo[1,5-a]pyrimidine-3-carboxamide (150 mg, 285.1 μmol, 32% yield) as a yellow solid. MS (ESI) m/z: 526.2 [M+H]⁺.

### Step 6:

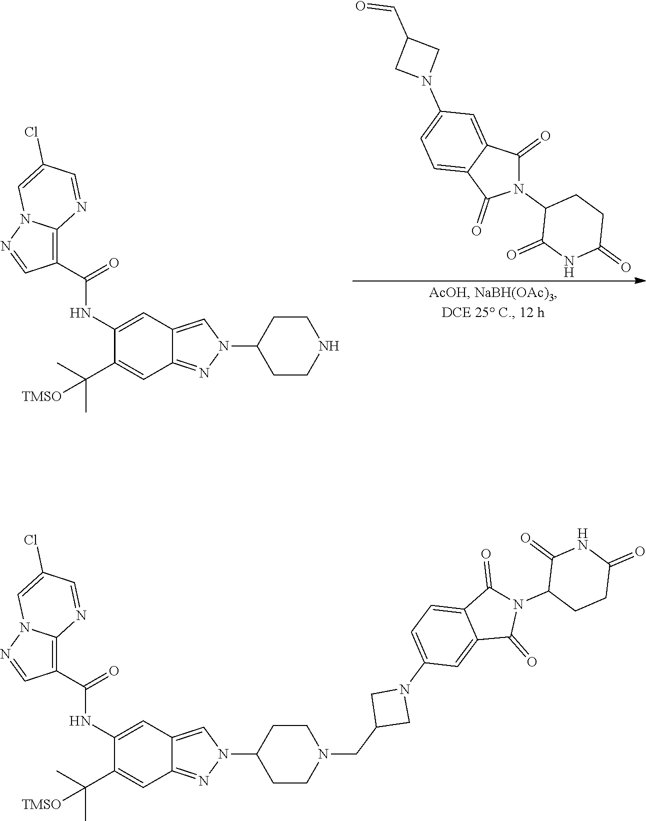

To a solution of 6-chloro-N-(2-(piperidin-4-yl)-6-(2-((trimethylsilyl)oxy)propan-2-yl)-2H-indazol-5-yl)pyrazolo[1,5-a]pyrimidine-3-carboxamide (100 mg, 190.1 μmol, 1 eq) and 1-(2-(2,6-dioxopiperidin-3-yl)-1,3-dioxoisoindolin-5-yl)azetidine-3-carbaldehyde (64.9 mg, 190.1 mol, 1 eq) in dichloroethane (2 mL) was added acetic acid (34.24 mg, 570.23 μmol, 32.61 μL, 3 eq). The mixture was stirred at 25° C. for 0.5 h. Sodium triacetoxyborohydride (161.14 mg, 760.3 mol, 4 eq) was then added to the mixture which was stirred at 25° C. for 11.5 h. The reaction mixture was quenched by the addition of water (10 mL) at 0° C., and then diluted with ethyl acetate (30 mL) and extracted with ethyl acetate (10 mL×3). The combined organic layers were washed with brine (10 mL×3), dried over anhydrous sodium sulfate, filtered, and concentrated under reduced pressure to give a residue. The residue was purified by preparative TLC (silicon dioxide, dichloromethane:methanol=10:1) to afford 6-chloro-N-(2-(1-((1-(2-(2,6-dioxopiperidin-3-yl)-1,3-dioxoisoindolin-5-yl)azetidin-3-yl)methyl)piperidin-4-yl)-6-(2-((trimethylsilyl)oxy)propan-2-yl)-2H-indazol-5-yl)pyrazolo[1,5-a]pyrimidine-3-carboxamide (150 mg, 176.2 μmol, 92% yield) as a yellow solid. MS (ESI) m/z: 761.3 [M−90]⁺.

### Step 7:

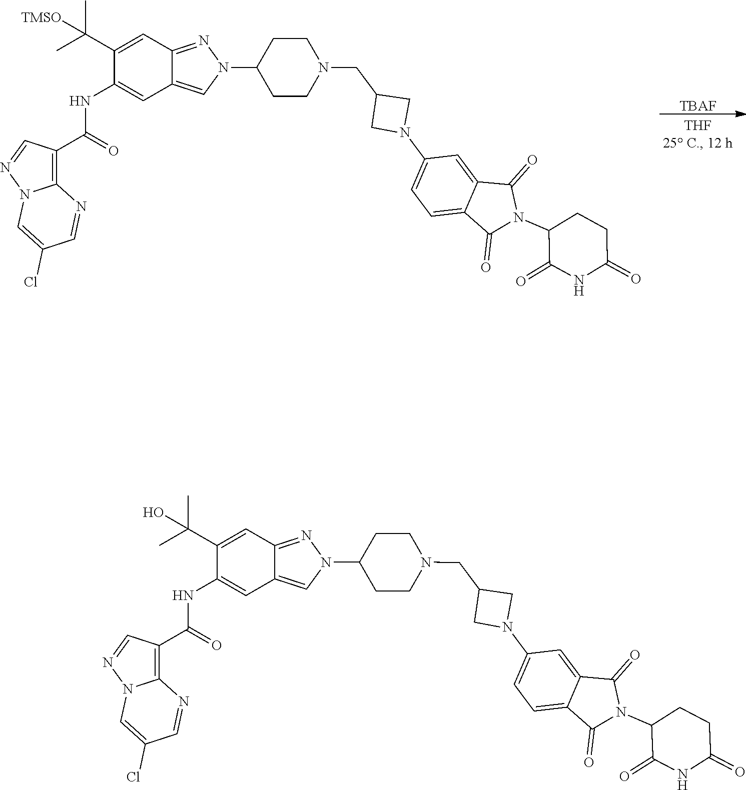

To a solution of 6-chloro-N-(2-(1-((1-(2-(2,6-dioxopiperidin-3-yl)-1,3-dioxoisoindolin-5-yl)azetidin-3-yl)methyl)piperidin-4-yl)-6-(2-((trimethylsilyl)oxy)propan-2-yl)-2H-indazol-5-yl)pyrazolo[1,5-a]pyrimidine-3-carboxamide (150 mg, 176.2 μmol, 1 eq) in tetrahydrofuran (1 mL) was added tetrabutylammonium fluoride solution (1 M, 528.5 μL, 3 eq). The mixture was stirred at 25° C. for 12 h. The reaction mixture was quenched by the addition of water (10 mL) at 0° C., and then diluted with ethyl acetate (30 mL) and extracted with ethyl acetate (10 mL×3). The combined organic layers were washed with brine (10 mL×3), dried over anhydrous sodium sulfate, filtered, and concentrated under reduced pressure to give a residue. The residue was purified by preparative HPLC to afford 6-chloro-N-(2-(1-((1-(2-(2,6-dioxopiperidin-3-yl)-1,3-dioxoisoindolin-5-yl)azetidin-3-yl)methyl)piperidin-4-yl)-6-(2-hydroxypropan-2-yl)-2H-indazol-5-yl)pyrazolo[1,5-a]pyrimidine-3-carboxamide (18.2 mg, 21.7 μmol, 12% yield, 93% purity), obtained as a yellow solid. MS (ESI) m/z: 761.3 [M−H₂O]⁺. ¹H NMR (400 MHz, DMSO-d₆) δ=11.31 (s, 1H), 11.08 (s, 1H), 9.77 (d, J=2.4 Hz, 1H), 8.85 (d, J=2.4 Hz, 1H), 8.68 (s, 1H), 8.40 (br d, J=10.8 Hz, 2H), 7.66 (br d, J=8.0 Hz, 1H), 7.56 (s, 1H), 6.81 (br s, 1H), 6.67 (br d, J=8.0 Hz, 1H), 5.76 (s, 1H), 5.06 (dd, J=5.6, 12.8 Hz, 1H), 4.59-4.08 (m, 3H), 3.74 (br s, 3H), 3.00 (br s, 3H), 2.96-2.82 (m, 1H), 2.94-2.82 (m, 1H), 2.62-2.54 (in, 411), 2.26-2.07 (m, 4H), 2.06-1.94 (m, 11H), 1.61 (s, 6H).

TABLE 10
The following compounds may be prepared in an analogous fashion as described for the
synthesis of Example 391.

| | | ¹H NMR |
|---|---|---|
| | | All spectra acquired at 400 MHz unless |
| Example | MS | otherwise indicated |
| Example 391 | See Above | |
| 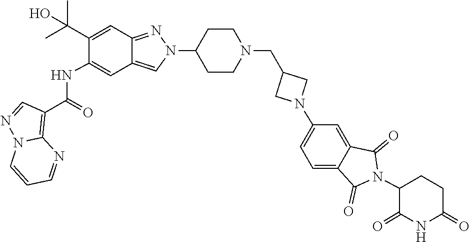 [SMILES: CC(C)(O)c1cc2nn(C3CCN(CC4CN(c5ccc6c(c5)C(=O)N(C5CCC(=O)NC5=O)C6=O)C4)CC3)cc2cc1NC(=O)c1cnn2cccnc12] | 745.3 | DMSO-d₆ δ = 11.25 (s, 1H), 11.07 (s, 1H), 9.31 (dd, J = 1.6, 7.0 Hz, 1H), 8.81 (dd, J = 1.6, 4.2 Hz, 1H), 8.65 (s, 1H), 8.40- 8.27 (m, 3H), 7.64 (d, J =8.4 Hz, 1H), 7.56 (s, 1H), 7.32-7.25 (m, 1H), 6.79 (d, J = 2.0 Hz, 1H), 6.66 (dd, J = 2.1, 8.3 Hz, 1H), 5.70 (br s, 1H), 5.05 (dd, J = 5.1, 12.8 Hz, 1H), 4.46 (s, 1H), 4.16 (t, J = 8.4 Hz, 2H), 3.76-3.70 (m, 2H), 3.00 (br d, J = 11.6 Hz, 3H), 2.71-2.65 (m, 3H), 2.34-2.30 (m, 2H), 2.27-2.17 (m, 1H), 2.15-2.08 (m, 4H), 2.01 (br d, J = 5.1 Hz, 1H), 1.60 (s, 6H) |
| Example 392 | | |
| 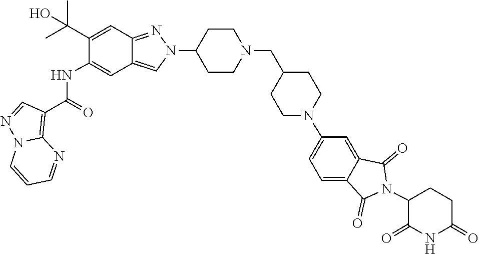 [SMILES: CC(C)(O)c1cc2nn(C3CCN(CC4CCN(c5ccc6c(c5)C(=O)N(C5CCC(=O)NC5=O)C6=O)CC4)CC3)cc2cc1NC(=O)c1cnn2cccnc12] | 773.3 | DMSO-d₆ δ = 11.25 (s, 1 H), 11.07 (s, 1 H), 9.31 (dd, J = 7.2, 1.6 Hz, 1 H), 8.81 (dd, J = 4.0, 1.6 Hz, 1 H), 8.65 (s, 1 H), 8.33-8.39 (m, 2 H), 7.66 (d, J = 8.4 Hz, 1 H), 7.56 (s, 1 H), 7.33 (s, 1 H), 7.24-7.30 (m, 2 H), 5.71 (s, 1 H), 5.06 (dd, J = 12.8, 5.2 Hz, 1 H), 4.34-4.55 (m, 1 H), 4.07 (d, J = 14.8 Hz, 2 H), 2.99 (t, J = 11.2 Hz, 6 H), 2.60 (s, 2 H), 2.19-2.26 (m, 2 H), 2.12 (s, 4 H), 1.84 (d, J = 11.6 Hz, 4 H), 1.60 (s, 6 H), 1.14-1.26 (m, 3 H). |
| Example 393 | | |
| 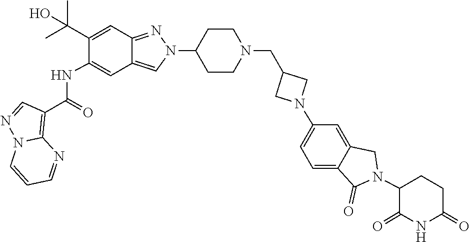 [SMILES: CC(C)(O)c1cc2nn(C3CCN(CC4CN(c5ccc6c(c5)CN(C5CCC(=O)NC5=O)C6=O)C4)CC3)cc2cc1NC(=O)c1cnn2cccnc12] | 731.2 | CDC3δ = 10.85 (s, 1H), 8.86-8.79 (m, 2H), 8.69 (dd, J = 1.6, 4.2 Hz, 1H), 8.49 (s, 1H), 7.98-7.84 (m, 2H), 7.76 (s, 1H), 7.71 (d, J = 8.4 Hz, 1H), 7.02 (dd, J = 4.0, 7.2 Hz, 1H), 6.47 (d, J = 8.8 Hz, 1H), 6.39 (s, 1H), 5.20 (dd, J = 4.8, 13.3 Hz, 1H), 4.40 (br d, J = 15.2 Hz, 2H), 4.25 (d, J = 15.6 Hz, 1H), 4.11 (t, J = 7.6 Hz, 2H), 3.67 (br t, J = 6.4 Hz, 2H), 3.06 (br s, 3H), 2.96-2.80 (m, 1H), 2.96-2.80 (m, 1H), 2.75 (br d, J = 6.4 Hz, 2H), 2.38-2.16 (m, 8H), 1.80 (s, 5H) |
| Example 394 | | |
| 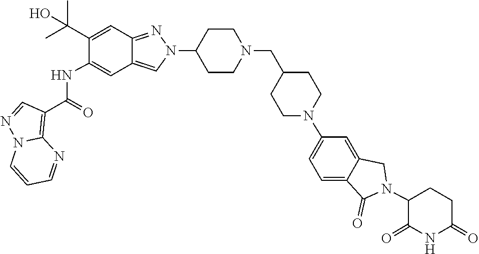 [SMILES: CC(C)(O)c1cc2nn(C3CCN(CC4CCN(c5ccc6c(c5)CN(C5CCC(=O)NC5=O)C6=O)CC4)CC3)cc2cc1NC(=O)c1cnn2cccnc12] | 759.3 | DMSO-d₆ δ = 11.24-11.30 (m, 1H), 10.95 (s, 1 H), 9.32 (dd, J = 7.2, 1.6 Hz, 1 H), 9.15 (s, 1 H), 8.81 (dd, J = 4.0, 1.6 Hz, 1 H), 8.66 (s, 1 H), 8.34-8.41 (m, 2 H), 7.50- 7.60 (m, 2 H), 7.26-7.32 (m, 1 H), 7.05- 7.12 (m, 2 H), 5.73 (s, 1 H), 5.05 (dd, J = 13.2, 5.2 Hz, 1 H), 4.74-4.96 (m, 1 H), 4.16-4.38 (m, 2 H), 3.94 (d, J = 12.4 Hz, 2 H), 3.75 (d, J = 12.0 Hz, 2 H), 3.06-3.26 (m, 3 H), 2.77-3.03 (m, 4 H), 2.53-2.64 (m, 2 H), 2.38 (d, J = 13.2 Hz, 4 H), 1.94- 2.16 (m, 2 H), 1.87 (d, J = 11.6 Hz, 2 H), 1.61 (s, 6H), 1.33 (d,J = 9.2 Hz, 2 H). |
| Example 395 | | |
| 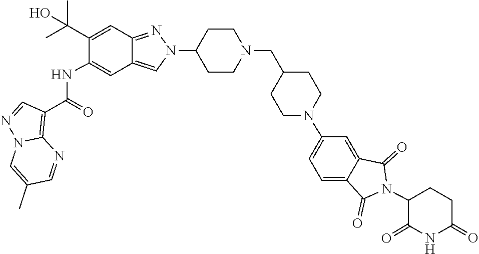 [SMILES: Cc1cnc2c(C(=O)Nc3cc4cn(C5CCN(CC6CCN(c7ccc8c(c7)C(=O)N(C7CCC(=O)NC7=O)C8=O)CC6)CC5)nc4cc3C(C)(C)O)cnn2c1] | 787.3 | DMSO-d₆ δ = 11.20 (s, 1 H), 11.07 (s, 1 H), 9.17 (s, 1H), 8.71 (d, J = 2.0 Hz, 1 H), 8.57 (s, 1 H), 8.35 (d, J = 10.4 Hz, 2 H), 7.66 (d, J = 8.8 Hz, 1 H), 7.56 (s, 1 H), 7.32 (d, J = 2.0 Hz, 1 H), 7.24 (dd, J = 2.0, 8.6 Hz, 1 H), 5.69 (s, 1 H), 5.06 (dd, J = 5.2, 12.8 Hz, 1 H), 4.44 (br s, 1 H), 4.06 (br d, J = 13.2 Hz, 2 H), 3.06-2.82 (m, 5 H), 2.63-2.53 (m, 2 H), 2.41 (s, 3 H), 2.22 (br d, J = 6.8 Hz, 2 H), 2.16-1.99 (m, 7 H), 1.83 (br d, J = 12.0 Hz, 3 H), 1.60 (s, 6H), 1.18 (br d, J = 10.8 Hz, 2 H) |
| Example 396 | | |

### Preparation of Example 397: N-(6-(1-((1-(2-(2,6-dioxopiperidin-3-yl)-7-methoxy-1,3-dioxoisoindolin-5-yl)azetidin-3-yl)methyl)piperidin-4-yl)-2-isopropoxy-7-oxo-6,7-dihydro-5H-pyrrolo[3,4-b]ipyridin-3-yl)pyrazolo [1,5-a]pyrimidine-3-carboxamide

### Step 1:

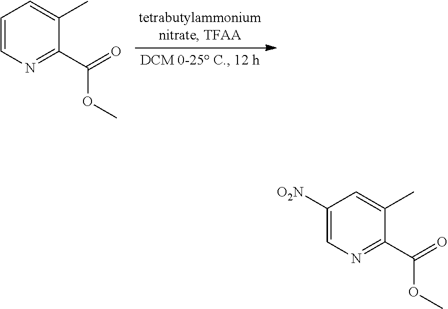

To a solution of methyl 3-methylpicolinate (6.5 g, 43.0 mmol, 1 eq) and tetrabutylammonium nitrate (14.40 g, 47.30 mmol, 1.1 eq) in dichloromethane (100 mL) was added trifluoroacetic anhydride (19.87 g, 94.60 mmol, 13.16 mL, 2.2 eq) at 0° C. and stirred at 25° C. for 12 h. The reaction mixture was quenched with water (100 mL), adjusted to pH=8 with a saturated solution of sodium bicarbonate at 0° C. and extracted with dichloromethane (80 mL×3). The combined organic layers were washed with brine (80 mL×2), dried over anhydrous sodium sulfate, filtered, and concentrated to give a residue. The residue was purified by silica gel column chromatography (petroleum ether/ethyl acetate=5/1 to 3/1) to afford methyl 3-methyl-5-nitropicolinate (7.2 g, 36.7 mmol, 85% yield) as a light yellow solid. MS (ESI) m/z: 197.1 [M+H]⁺.

### Step 2:

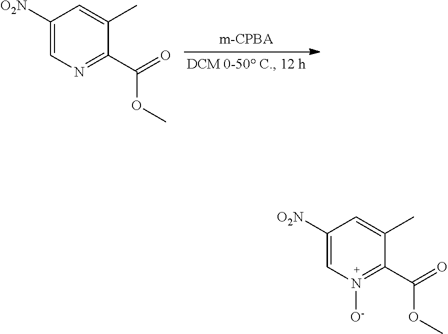

To a solution of methyl 3-methyl-5-nitropicolinate (7.2 g, 36.70 mmol, 1 eq) in dichloromethane (80 mL) was added m-chloroperbenzoic acid (11.18 g, 55.06 mmol, 85% purity, 1.5 eq) at 0° C. and stirred at 50° C. for 12 h. The reaction mixture was quenched with water (60 mL), adjusted to pH=8 with a saturated aqueous solution of sodium bicarbonate at 0° C., and extracted with dichloromethane (80 mL×4). The combined organic layers were washed with brine (30 mL×2), dried over anhydrous sodium sulfate, filtered, and concentrated to give a residue. The residue was purified by silica gel column chromatography (petroleum ether/ethyl acetate=5/1 to 1/1) to afford 2-(methoxycarbonyl)-3-methyl-5-nitropyridine 1-oxide (4 g, 18.85 mmol, 51% yield) obtained as a light yellow solid. MS (ESI) m/z: 213.0 [M+H]⁺. ¹H NMR (400 MHz, CDCl₃) δ: 8.89 (d, J=1.2 Hz, 1H) 7.87-7.93 (m, 1H) 4.06 (s, 3H) 2.43 (s, 3H).

### Step 3:

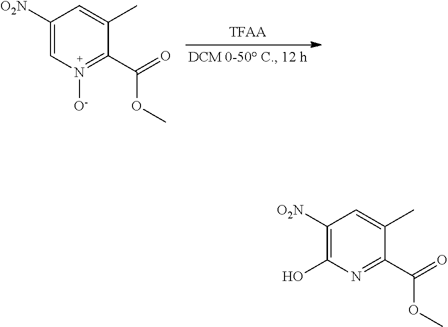

To a solution of 2-(methoxycarbonyl)-3-methyl-5-nitropyridine 1-oxide (4 g, 18.85 mmol, 1 eq) in N,N-dimethylformamide (40 mL) was added trifluoroacetic anhydride (31.68 g, 150.83 mmol, 20.98 mL, 8 eq) at 0° C. and stirred at 50° C. for 12 h. The mixture was poured into ice-water (80 mL). The aqueous phase was extracted with ethyl acetate (80 mL×6). The combined organic phase was washed with brine (30 mL×2), dried with anhydrous sodium sulfate, filtered, and concentrated under vacuum. The crude product was triturated with petroleum ether (100 mL) at 25° C. for 0.5 h to yield methyl 6-hydroxy-3-methyl-5-nitropicolinate (2.5 g, 11.78 mmol, 62% yield) as a yellow solid. MS (ESI) m/z: 213.0 [M+H]⁺. ¹H NMR (400 MHz, DMSO-d₆) δ: 12.80 (s, 1H) 8.40 (s, 1H) 3.88 (s, 3H) 2.30 (s, 3H).

### Step 4:

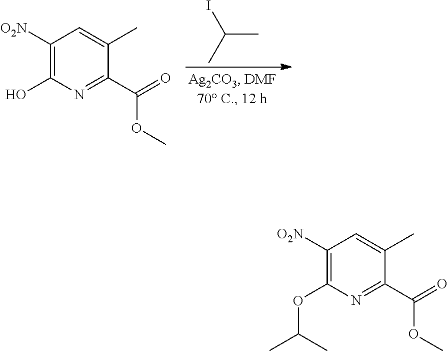

To a solution of methyl 6-hydroxy-3-methyl-5-nitropicolinate (0.9 g, 4.24 mmol, 1 eq) and 2-iodopropane (1.44 g, 8.48 mmol, 848.38 μL, 2 eq) in DMF (10 mL) was dropwise added silver carbonate (1.75 g, 6.36 mmol, 288.59 μL, 1.5 eq) and stirred at 70° C. for 12 h. The mixture was then poured into ice-water (30 mL). The aqueous phase was extracted with ethyl acetate (30 mL×3). The combined organic phase was washed with brine (20 mL×2), dried with anhydrous sodium sulfate, filtered, and concentrated under vacuum. The residue was purified by silica gel column chromatography (petroleum ether/ethyl acetate=5/1) to afford methyl 6-isopropoxy-3-methyl-5-nitropicolinate (0.7 g, 2.75 mmol, 64% yield) as a light yellow solid. ¹H NMR (400 MHz, CDCl₃) δ: 8.08 (s, 1H) 5.51 (m, 1H) 3.97 (s, 3H) 2.49 (s, 3H) 1.41 (d, J=6.0 Hz, 6H).

### Step 5:

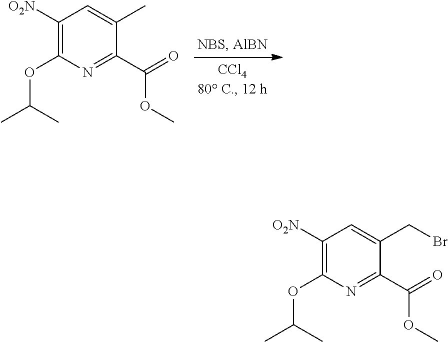 [SMILES: COC(=O)c1nc(OC(C)C)c([N+](=O)[O-])cc1C ; COC(=O)c1nc(OC(C)C)c([N+](=O)[O-])cc1CBr]

To a solution of methyl 6-isopropoxy-3-methyl-5-nitropicolinate (1.3 g, 5.11 mmol, 1 eq) and N-bromosuccinimide (910.1 mg, 5.11 mmol, 1 eq) in carbon tetrachloride (10 mL) was added azobisisobutyronitrile (71.37 mg, 434.63 μmol, 0.085 eq) and stirred at 80° C. for 12 h. The mixture was concentrated to give a residue. The residue was purified by silica gel column chromatography (petroleum ether/ethyl acetate=50/1 to 20/1) to afford methyl 3-(bromomethyl)-6-isopropoxy-5-nitropicolinate (0.8 g, 2.40 mmol, 46% yield) as a light yellow solid.

### Step 6:

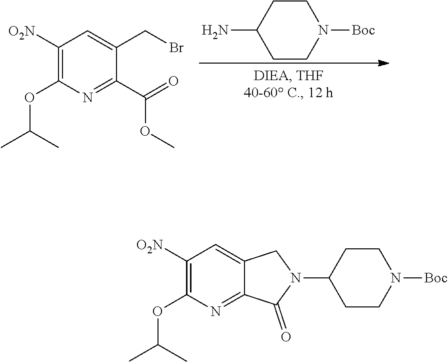 [SMILES: COC(=O)c1nc(OC(C)C)c([N+](=O)[O-])cc1CBr ; CC(C)(C)OC(=O)N1CCC(N)CC1 ; CC(C)Oc1nc2c(cc1[N+](=O)[O-])CN(C1CCN(C(=O)OC(C)(C)C)CC1)C2=O]

To a solution of tert-butyl 4-aminopiperidine-1-carboxylate (1.44 g, 7.20 mmol, 3 eq) and N,N-diisopropylethylamine (1.55 g, 12.01 mmol, 2.09 mL, 5 eq) in tetrahydrofuran (10 mL) was dropwise added methyl 3-(bromomethyl)-6-isopropoxy-5-nitropicolinate (0.8 g, 2.40 mmol, 1 eq) in tetrahydrofuran (5 mL) at 40° C. and stirred at 60° C. for 12 h. The mixture was concentrated to give a residue. The residue was purified by silica gel column chromatography (petroleum ether/ethyl acetate=20/1 to 2/1) to afford tert-butyl 4-(2-isopropoxy-3-nitro-7-oxo-5H-pyrrolo[3,4-b]pyridin-6(7H)-yl)piperidine-1-carboxylate (0.55 g, 1.31 mmol, 54% yield) as a light yellow solid. MS (ESI) m/z: 365.1 [M-tBu]⁺.

### Step 7:

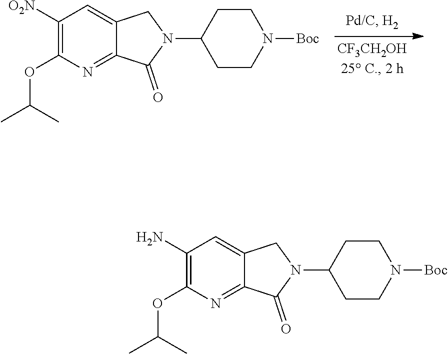 [SMILES: CC(C)Oc1nc2c(cc1[N+](=O)[O-])CN(C1CCN(C(=O)OC(C)(C)C)CC1)C2=O ; CC(C)Oc1nc2c(cc1N)CN(C1CCN(C(=O)OC(C)(C)C)CC1)C2=O]

To a solution of tert-butyl 4-(2-isopropoxy-3-nitro-7-oxo-5H-pyrrolo[3,4-b]pyridin-6(7H)-yl)piperidine-1-carboxylate (0.5 g, 1.19 mmol, 1 eq) in trifluoroethanol (10 mL) was added palladium on carbon (10%, 500 mg) under a nitrogen atmosphere. The suspension was degassed and purged with hydrogen in 3 cycles. The mixture was stirred under hydrogen (15 psi) at 25° C. for 2 h. The reaction mixture was filtered and washed with ethanol (20 mL×2). The collected filtrate was concentrated to give tert-butyl 4-(3-amino-2-isopropoxy-7-oxo-5H-pyrrolo[3,4-b]pyridin-6(7H)-yl)piperidine-1-carboxylate (0.4 g, 1.02 mmol, 86% yield) obtained as a light yellow solid which was used in the next step without further purification. MS (ESI) m/z: 391.2 [M+H]⁺.

### Step 8:

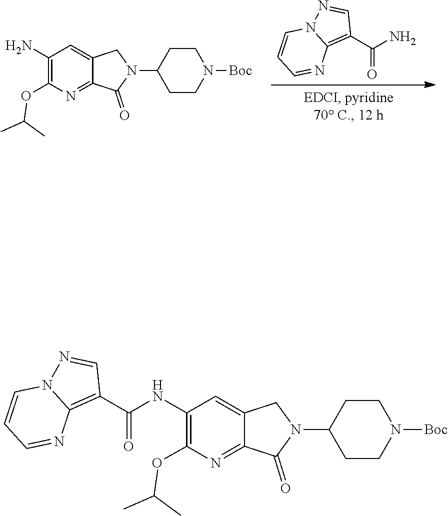 [SMILES: CC(C)Oc1nc2c(cc1N)CN(C1CCN(C(=O)OC(C)(C)C)CC1)C2=O ; NC(=O)c1cnn2cccnc12 ; CC(C)Oc1nc2c(cc1NC(=O)c1cnn3cccnc13)CN(C1CCN(C(=O)OC(C)(C)C)CC1)C2=O]

To a solution of tert-butyl 4-(3-amino-2-isopropoxy-7-oxo-5H-pyrrolo[3,4-b]pyridin-6(7H)-yl)piperidine-1-carboxylate (370 mg, 947.56 μmol, 1 eq) and pyrazolo[1,5-a]pyrimidine-3-carboxylic acid (463.74 mg, 2.84 mmol, 3 eq) in pyridine (10 mL) was added 1-(3-dimethylaminopropyl)-3-ethylcarbodiimide hydrochloride (908.25 mg, 4.74 mmol, 5 eq) and stirred at 70° C. for 12 h. The mixture was concentrated to give a residue. The residue was purified by preparative thin-layer chromatography (petroleum ether/ethyl acetate=0/1) to afford tert-butyl 4-(2-isopropoxy-7-oxo-3-(pyrazolo[1,5-a]pyrimidine-3-carboxamido)-5H-pyrrolo[3,4-b]pyridin-6(7H)-yl)piperidine-1-carboxylate (0.27 g, 504.1 μmol, 53% yield) was obtained as a yellow solid. MS (ESI) m/z: 536.3 [M+H]⁺.

### Step 9:

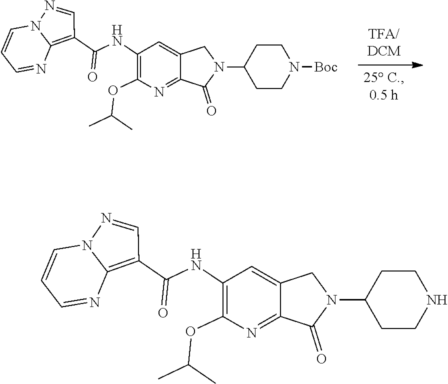 [SMILES: CC(C)Oc1nc2c(cc1NC(=O)c1cnn3cccnc13)CN(C1CCN(C(=O)OC(C)(C)C)CC1)C2=O ; CC(C)Oc1nc2c(cc1NC(=O)c1cnn3cccnc13)CN(C1CCNCC1)C2=O]

To a solution of tert-butyl 4-(2-isopropoxy-7-oxo-3-(pyrazolo[1,5-a]pyrimidine-3-carboxamido)-5H-pyrrolo[3,4-b]pyridin-6(7H)-yl)piperidine-1-carboxylate (0.25 g, 466.77 μmol, 1 eq) in dichloromethane (3 mL) was added a solution of hydrochloric acid in methanol (4 M, 10 mL). The reaction mixture was stirred at 25° C. for 0.5 h after which it was concentrated to give a residue. The residue was diluted with dichloromethane/methyl alcohol=10/1 (30 mL), adjusted to pH=8 with ammonium hydroxide (33%) at 0° C., dried over anhydrous sodium sulfate, filtered, and concentrated to yield N-(2-isopropoxy-7-oxo-6-(piperidin-4-yl)-6,7-dihydro-5H-pyrrolo[3,4-b]pyridin-3-yl)pyrazolo[1,5-a]pyrimidine-3-carboxamide (0.2 g, 459.27 μmol, 98% yield) as a yellow solid used directly in the next step without further purification. MS (ESI) m/z: 436.2 [M+H]⁺.

### Step 10:

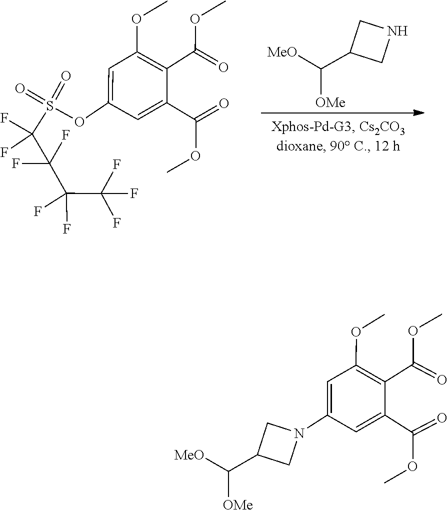 [SMILES: COC(=O)c1cc(OS(=O)(=O)C(F)(F)C(F)(F)C(F)(F)C(F)(F)F)cc(OC)c1C(=O)OC ; COC(OC)C1CNC1 ; COC(=O)c1cc(N2CC(C(OC)OC)C2)cc(OC)c1C(=O)OC]

A mixture of dimethyl 3-methoxy-5-(((perfluorobutyl)sulfonyl)oxy)phthalate (5 g, 9.57 mmol, 1 eq), 3-(dimethoxymethyl)azetidine (1.51 g, 11.49 mmol, 1.2 eq), cesium carbonate (9.36 g, 28.72 mmol, 3 eq), XPhos Pd G3 (810.32 mg, 957.32 μmol, 0.1 eq) in dioxane (100 mL) was degassed and purged with nitrogen in 3 cycles, and then the mixture was stirred at 90° C. for 12 h under a nitrogen atmosphere. The reaction mixture was quenched by addition of water (200 mL) at 15° C., and then diluted with ethyl acetate (100 mL) and extracted with ethyl acetate (100 mL×2). The combined organic layers were washed with brine (200 mL×2), dried over anhydrous sodium sulfate, filtered and concentrated under reduced pressure to give a residue. The residue was purified by silica gel column chromatography (petroleum ether/ethyl acetate=10/1 to 1/1) to afford dimethyl 5-(3-(dimethoxymethyl)azetidin-1-yl)-3-methoxyphthalate (1.5 g, 4.24 mmol, 44% yield) as a yellow oil. (ESI) m/z: 354.2 [M+H]⁺.

### Step 11:

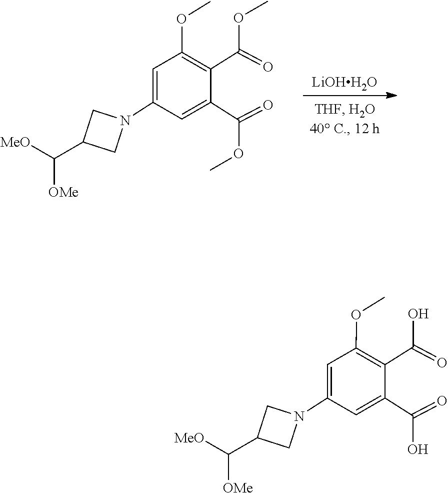

To a solution of dimethyl 5-(3-(dimethoxymethyl)azetidin-1-yl)-3-methoxyphthalate (1.5 g, 4.24 mmol, 1 eq) in tetrahydrofuran (12 mL) was added lithium hydroxide (890.65 mg, 21.22 mmol, 5 eq) and water (4 mL). The mixture was stirred at 40° C. for 12 h. Hydrochloric acid (1 M, 5 mL) was added to the reaction mixture to adjust the pH to about 4-5, then the mixture was extracted with ethyl acetate (20 mL×2). The combined organic layers were dried over anhydrous sodium sulfate, filtered, and concentrated under reduced pressure to afford 5-(3-(dimethoxymethyl)azetidin-1-yl)-3-methoxyphthalic acid (760 mg, 2.34 mmol, 55% yield) as a yellow solid which was used directly for the next step. MS (ESI) m/z: 326.1 [M+H]⁺.

### Step 12:

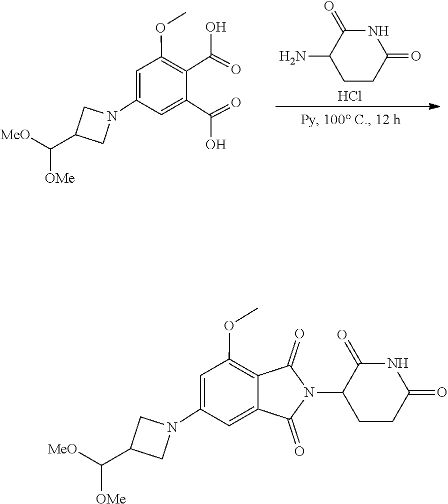 [SMILES: COc1cc(N2CC(C(OC)OC)C2)cc(C(=O)O)c1C(=O)O ; NC1CCC(=O)NC1=O ; Cl ; COc1cc(N2CC(C(OC)OC)C2)cc2c1C(=O)N(C1CCC(=O)NC1=O)C2=O]

A solution of 5-[3-(dimethoxymethyl)azetidin-1-yl]-3-methoxy-phthalic acid (760 mg, 2.34 mmol, 1 eq) and 3-aminopiperidine-2,6-dione (576.78 mg, 3.50 mmol, 1.5 eq, hydrochloric acid) in pyridine (10 mL) was stirred at 100° C. for 12 h. The reaction mixture was concentrated under reduced pressure. The residue was purified by preparative thin-layer chromatography (dichloromethane:methyl alcohol=10:1) to afford 6-(3-(dimethoxymethyl)azetidin-1-yl)-2-(2,6-dioxopiperidin-3-yl)-4-methoxyisoindoline-1,3-dione (200 mg, 479.14 μmol, 21% yield) as a yellow solid, MS (ESI) m/z: 418.1 [M+H]⁺.

### Step 13:

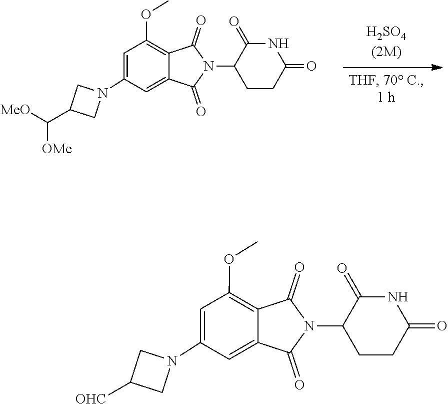 [SMILES: COc1cc(N2CC(C(OC)OC)C2)cc2c1C(=O)N(C1CCC(=O)NC1=O)C2=O ; COc1cc(N2CC(C=O)C2)cc2c1C(=O)N(C1CCC(=O)NC1=O)C2=O]

To a solution of 6-(3-(dimethoxymethyl)azetidin-1-yl)-2-(2,6-dioxopiperidin-3-yl)-4-methoxyisoindoline-1,3-dione (80 mg, 191.66 μmol, 1 eq) in tetrahydrofuran (2 mL) was added sulfuric acid (2 M, 2 mL, 17.82 eq). The mixture was stirred at 70° C. for 1 h. A saturated solution of sodium bicarbonate was added to adjust the pH to 8. Then the mixture was extracted with dichloromethane 40 mL (20 mL×2). The combined organic layers were dried over sodium sulfate, filtered, and concentrated under reduced pressure yielding 1-(2-(2,6-dioxopiperidin-3-yl)-7-methoxy-1,3-dioxoisoindolin-5-yl)azetidine-3-carbaldehyde (70 mg, 188.50 μmol, 98% yield) as a yellow solid which was used as is without further purification. MS (ESI) m/z: 372.1 [M+H]⁺.

### Step 14:

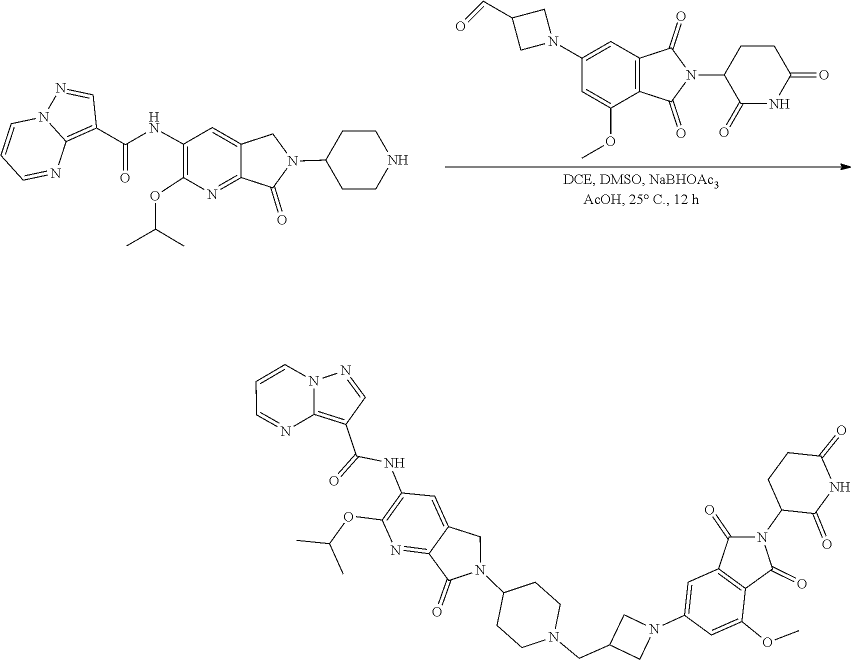 [SMILES: CC(C)Oc1nc2c(cc1NC(=O)c1cnn3cccnc13)CN(C1CCNCC1)C2=O ; COc1cc(N2CC(C=O)C2)cc2c1C(=O)N(C1CCC(=O)NC1=O)C2=O ; COc1cc(N2CC(CN3CCC(N4Cc5cc(NC(=O)c6cnn7cccnc67)c(OC(C)C)nc5C4=O)CC3)C2)cc2c1C(=O)N(C1CCC(=O)NC1=O)C2=O]

To a solution of N-(2-isopropoxy-7-oxo-6-(piperidin-4-yl)-6,7-dihydro-5H-pyrrolo[3,4-b]pyridin-3-yl)pyrazolo[1,5-a]pyrimidine-3-carboxamide (150 mg, 0.34 mmol, 1 eq), 1-(2-(2,6-dioxopiperidin-3-yl)-7-methoxy-1,3-dioxoisoindolin-5-yl)azetidine-3-carbaldehyde (192 mg, 0.52 mmol, 1.5 eq) in dichloroethane (5 mL) and dimethyl sulfoxide (5 mL) was added acetic acid (20.7 mg, 0.34 mmol, 0.02 mL, 1 eq). The reaction mixture was stirred at 25° C. for 1 h, and then sodium triacetoxyborohydride (219 mg, 1.03 mmol, 3 eq) was added. The reaction was stirred at 25° C. for 11 h. To the reaction mixture was added water (30 mL) and the mixture was extracted with dichloromethane (30 mL×3). The combined organic phase was washed with brine (20 mL), dried over anhydrous sodium sulfate, filtered, and concentrated in vacuo. The crude product was purified by preparative HPLC (column: Phenomenex luna C18 250×50 mm×10 m; mobile phase: [water(0.225% formic acid)-acetonitrile]; B %: 10%-36%, 19 min as additive) to yield N-(6-(1-((1-(2-(2,6-dioxopiperidin-3-yl)-7-methoxy-1,3-dioxoisoindolin-5-yl)azetidin-3-yl)methyl)piperidin-4-yl)-2-isopropoxy-7-oxo-6,7-dihydro-5H-pyrrolo[3,4-b]pyridin-3-yl)pyrazolo[1,5-a]pyrimidine-3-carboxamide (50.5 mg, 17% yield, 95% purity, formate) as a yellow solid. MS (ESI) m/z: 791.2 [M+H]⁺. ¹H NMR (400 MHz, DMSO-d₆) δ: 11.04 (s, 1H), 10.79 (s, 1H), 9.41 (d, J=7.2 Hz, 1H), 8.96 (s, 1H), 8.92 (dd, J=4.4, 1.6 Hz, 1H), 8.76 (s, 1H), 8.20 (s, 1H), 7.37 (dd, J=6.8, 4.0 Hz, 1H), 6.40 (s, 1H), 6.12 (s, 1H), 5.44 (q, J=6.4 Hz, 1H), 4.98 (dd, J=12.8, 5.2 Hz, 1H), 4.42 (s, 2H), 4.15 (t, J=8.0 Hz, 2H), 4.01 (t, J=11.6 Hz, 1H), 3.89 (s, 3H), 3.72 (dd, J=8.4, 5.6 Hz, 2H), 2.96 (d, J=10.4 Hz, 2H), 2.58-2.66 (m, 2H), 2.54 (s, 1H), 2.06-2.15 (m, 2H), 1.98 (dd, J=14.8, 8.4 Hz, 2H), 1.66-1.84 (m, 4H), 1.49 (d, J=6.0 Hz, 6H), 1.23 (s, 2H).

TABLE 11
The following compounds may be prepared in an analogous fashion as described for the
synthesis of Example 397 and the procedures described in Schemes 1-7.

| | | ¹H NMR |
|---|---|---|
| | | All spectra acquired at 400 MHz unless |
| Example | MS | otherwise indicated |
| Example 397 | See Above | |
| 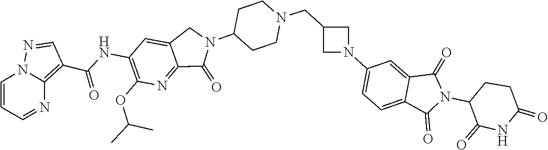 [SMILES: CC(C)Oc1nc2c(cc1NC(=O)c1cnn3cccnc13)CN(C1CCN(CC3CN(c4ccc5c(c4)C(=O)N(C4CCC(=O)NC4=O)C5=O)C3)CC1)C2=O] | 761.3 | DMSO-d₆ δ = 11.07 (s, 1 H) 10.76 (s, 1 H) 9.39 (dd, J = 6.8, 1.6 Hz, 1 H) 8.94 (s, 1 H) 8.90 (dd, J = 4.4, 1.6 Hz, 1 H) 8.74 (s, 1 H) 7.61 (d, J = 8.0 Hz, 1 H) 7.34-7.38 (m, 1 H) 6.75 (d, J = 1.6 Hz, 1 H) 6.62 (dd, J = 8.4, 2.0 Hz, 1 H) 5.43 (m, 1 H) 5.05 (dd, J = 12.8, 5.6 Hz, 1 H) 4.41 (s, 2 H) 4.12 (m, 2 H) 4.00 (m, 1 H) 3.66-3.75 (m, 2 H) 2.83-2.99 (m, 4 H) 2.53-2.69 (m, 4 H) 2.00-2.13 (m, 3 H) 1.67-1.83 (m, 4 H) |
| Example 398 | | 1.49 (d, J = 6.0 Hz, 6 H) |
| 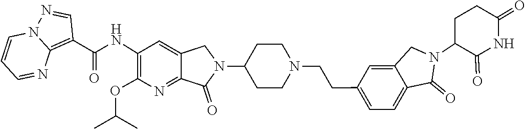 [SMILES: CC(C)Oc1nc2c(cc1NC(=O)c1cnn3cccnc13)CN(C1CCN(CCc3ccc4c(c3)CN(C3CCC(=O)NC3=O)C4=O)CC1)C2=O] | 706.3 | DMSO-d₆ δ = 10.98 (s, 1 H) 10.78 (s, 1 H) 9.42 (m, 1 H) 8.94 (br s, 2 H) 8.76 (s, 1 H) 8.28 (s, 1 H) 7.65 (br d, J = 7.83 Hz, 1 H) 7.49 (s, 1 H) 7.40 (d, J = 7.6 Hz, 2 H) 5.39-5.47 (m, 1 H) 5.10 (br d, J= 13.6 Hz, 1 H) 4.40-4.47 (m, 3 H) 4.27-4.33 (m, 1 H) 4.02 (br s, 1H) 3.06 (br d, J = 10.8 Hz, 2H) 2.89 (br s, 4 H) 2.61-2.68 (m, 2 H) 2.08- |
| | | 2.17 (m, 2 H) 2.01 (br s, 2 H) 1.70-1.81 |
| Example 399 | | (m, 4 H) 1.49 (br d, J = 6.0 Hz, 6 H) |
| 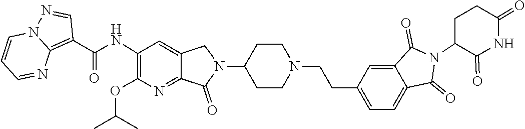 [SMILES: CC(C)Oc1nc2c(cc1NC(=O)c1cnn3cccnc13)CN(C1CCN(CCc3ccc4c(c3)C(=O)N(C3CCC(=O)NC3=O)C4=O)CC1)C2=O] | 720.3 | DMSO-d₆ δ = 11.13 (s, 1 H), 10.77 (s, 1 H), 9.42 (dd, J = 6.8, 1.6 Hz, 1 H), 8.90- 8.96 (m, 2 H), 8.76 (s, 1 H), 8.18-8.24 (m, 1 H), 8.22 (s, 1 H), 7.83-7.87 (m, 2 H), 7.73-7.80 (m, 1 H), 7.34-7.40 (m, 1 H), 5.43 (dt, J = 12.4, 6.4 Hz, 1 H), 5.15 (dd, J = 12.8,5.2 Hz, 1H), 4.40 (s,2H), 3.98- 4.05 (m, 1 H), 3.06 (d, J = 10.8 Hz, 2 H), |
| | | 2.98 (t, J = 7.2 Hz, 2 H), 2.85-2.92 (m, 1 |
| | | H), 2.56-2.68 (m, 4 H), 2.05-2.17 (m, 3 |
| | | H), 1.67-1.81 (m, 4 H), 1.49 (d, J = 6.0 |
| Example 400 | | Hz, 6 H). |
| 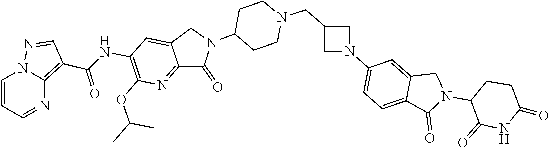 [SMILES: CC(C)Oc1nc2c(cc1NC(=O)c1cnn3cccnc13)CN(C1CCN(CC3CN(c4ccc5c(c4)CN(C4CCC(=O)NC4=O)C5=O)C3)CC1)C2=O] | 747.3 | DMSO-d₆ δ = 10.93 (s, 1H), 10.78 (s, 1H), 9.42 (dd, 1H, J = 1.6, 7.2 Hz), 8.9-9.0 (m, 2H), 8.76 (s, 1H), 8.17 (s, 1H), 7.47 (d, 1H, J = 8.4 Hz), 7.37 (dd, 1H, J = 4.4, 6.8 Hz), 6.4-6.5 (m, 2H), 5.3-5.5 (m, 1H), 5.0- 5.1 (m, 1H), 4.41 (s, 2H), 4.2-4.3 (m, 1H), 4.1-4.2 (m, 1H), 4.0-4.1 (m, 3H), 3.56 (1, 2H, J = 5.2 Hz), 2.9-2.9 (m, 1H), 2.8-3.0 (m, 5H), 2.6-2.6 (m, 3H), 2.0-2.2 (m, 2H), 1.9-2.0 (m, 1H), 1.6-1.9 (m, 5H), 1.48 (d, |
| Example 401 | | 7H, J = 6.0 Hz) |
| 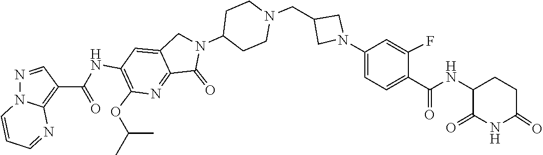 [SMILES: CC(C)Oc1nc2c(cc1NC(=O)c1cnn3cccnc13)CN(C1CCN(CC3CN(c4ccc(C(=O)NC5CCC(=O)NC5=O)c(F)c4)C3)CC1)C2=O] | 753.2 | DMSO-d₆ δ = 10.83 (s, 1H), 10.78 (s, 1H), 9.42 (dd, J = 1.6, 7.2 Hz, 1H), 8.95 (s, 1H), 9.45 (dd, J = 1.6, 4.0 Hz, 1H), 8.76 (s, 1H), 7.97 (t, J = 7.2 Hz, 1H), 7.61 (t, J = 8.4 Hz, 1H), 7.38 (dd, J = 4.4, 6.8 Hz, 1H), 6.31-6.26 (m, 1H), 6.25-6.19 (m, 1H), 5.48-5.40 (m, 1H), 4.75-4.69 (m, 1H), 4.42 (s, 2H), 4.02 (t, J = 9.6 Hz, 2H), 3.60-3.56 (m, 2H), 2.97-2.93 (m, 2H), 2.80-2.72 (m, 2H), 2.63-2.61 (m, 2H), |
| | | 2.14-2.06 (m, 3H), 2.04-1.97 (m, 2H), |
| Example 402 | | 1.87-1.65 (m, 5H), 1.49(d, J = 6.4 Hz, 6H) |
| 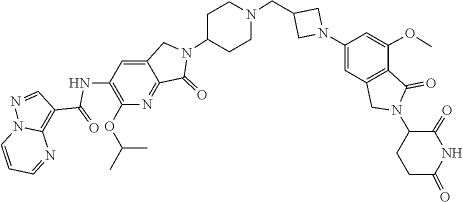 [SMILES: COc1cc(N2CC(CN3CCC(N4Cc5cc(NC(=O)c6cnn7cccnc67)c(OC(C)C)nc5C4=O)CC3)C2)cc2c1C(=O)N(C1CCC(=O)NC1=O)C2] | 777.3 | DMSO-d₆ δ = 10.90 (s, 1H), 10.78 (s, 1H), 9.40 (dd, J = 6.8, 1.6 Hz, 1H), 8.95 (s, 1H), 8.92 (dd, J = 4.4, 1.60 Hz, 1H), 8.76 (s, 1H), 8.17 (s, 1H), 7.34-7.40 (m, 1H), 6.05 (s, 1H), 5.92 (d, J = 1.2 Hz, 1H), 5.50-5.37 (m, 1H), 4.95 (dd, J = 13.2, 5.2 Hz, 1H), 4.42 (s, 2H), 4.17-4.24 (m, 1H), 4.10 (s, 1H), 3.97-4.07 (m, 3H), 3.81 (s, 3H), 3.59-3.57 (m, 2H), 2.94-2.97 (m,2H), 2.82-2.91 (m, 1H), 2.58-2.69 (m, 3H), 2.53-2.57 (m, 1H), 2.24-2.38 (m, 1H), 2.05- 2.17 (m, 2H), 1.86-1.96 (m, 1H), 1.63-1.86 (m, 4H),1.50(d, J = 6.0 Hz, 6H) |
| Example 403 | | |

### Preparation of Example 404: N-[6-[1-[1-[2-(2,6-dioxo-3-piperidyl)-1-oxo-isoindolin-5-yl]azetidin-3-yl]-4-piperidyl]-2-morpholino-7-oxo-5H-pyrrolo[3,4-b]pyridin-3-yl]pyrazolo[1,5-a]pyrimidine-3-carboxamide

### Step 1:

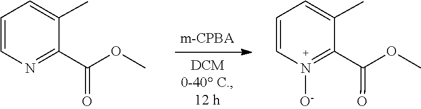

To a solution of methyl methyl 3-methylpicolinate (20 g, 132.31 mmol, 1 eq) in dichloromethane (300 mL) was added m-chloroperoxybenzoic acid (40.29 g, 198.46 mmol, 85% purity, 1.5 eq) at 0° C. which was subsequently allowed to stir at 40° C. for 12 h. The reaction mixture was quenched with water (300 mL), adjusted to pH=8 with saturated aqueous solution of sodium bicarbonate at 0° C., and extracted with dichloromethane (100 mL×4). The combined organic layers were washed with brine (80 mL×2), dried over anhydrous sodium sulfate, filtered, and concentrated to give a residue. The residue was purified by silica gel column chromatography (eluted with dichloromethane/methanol=100/1 to 20/1) to afford 2-(methoxycarbonyl)-3-methylpyridine 1-oxide (20 g, 119.64 mmol, 90% yield) as a light yellow oil. MS (ESI) m/z: 168.6 [M+H]⁺. ¹H NMR (400 MHz, CDCl₃) δ: 8.08 (d, J=6.4 Hz, 1H) 7.17-7.23 (m, 1H) 7.10-7.15 (m, 1H) 4.01 (s, 3H) 2.29 (s, 3H).

### Step 2:

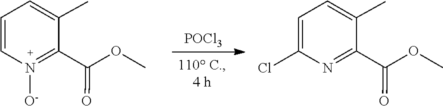

To a solution of phosphoryl trichloride (132.00 g, 860.88 mmol, 80 mL, 7.20 eq) was added 2-(methoxycarbonyl)-3-methylpyridine 1-oxide (20 g, 119.64 mmol, 1 eq) at 25° C. and stirred at 110° C. for 4 h. The mixture was concentrated to give a residue. The residue was quenched with ice-water (200 mL), adjusted pH of 7 with a saturated aqueous solution of sodium bicarbonate and extracted with ethyl acetate (150 mL×3). The combined organic phase was washed with brine (60 mL×2), dried with anhydrous sodium sulfate, filtered, and concentrated under vacuum. The residue was purified by silica gel column chromatography (petroleum ether/ethyl acetate=10/1 to 6/1) to afford methyl 6-chloro-3-methylpicolinate (15 g, 80.82 mmol, 67% yield) as a light yellow solid. MS (ESI) m/z: 186.0 [M+H]⁺. ¹H NMR (400 MHz, CDCl₃) δ: 7.57 (d, J=8.0 Hz, 1H) 7.36 (d, J=8.0 Hz, 1H) 3.95 (s, 3H) 2.55 (s, 3H).

### Step 3:

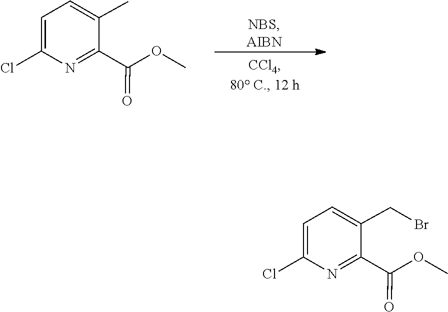

To a solution of methyl 6-chloro-3-methylpicolinate (6 g, 32.33 mmol, 1 eq) and NBS (5.75 g, 32.33 mmol, 1 eq) in perchloromethane (120 mL) was added azobisisobutyronitrile (451.20 mg, 2.75 mmol, 0.085 eq). The reaction mixture was stirred at 80° C. for 12 h. The mixture was concentrated to give a residue. The residue was purified by silica gel column chromatography (petroleum ether/ethyl acetate=20/1 to 3/1) to afford methyl 3-(bromomethyl)-6-chloropicolinate (5 g, 18.90 mmol, 58% yield), as a light yellow solid. MS (ESI) m/z: 265.9 [M+H]⁺. ¹H NMR (400 MHz, CDCl₃) δ: 7.83 (d, J=8.4 Hz, 1H) 7.47 (d, J=8.4 Hz, 1H) 4.86 (s, 2H) 3.99 (s, 3H).

### Step 4:

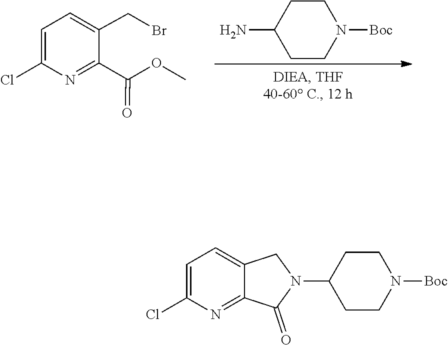 [SMILES: COC(=O)c1nc(Cl)ccc1CBr ; CC(C)(C)OC(=O)N1CCC(N)CC1 ; CC(C)(C)OC(=O)N1CCC(N2Cc3ccc(Cl)nc3C2=O)CC1]

To a solution of tert-butyl 4-aminopiperidine-1-carboxylate (9.09 g, 45.37 mmol, 3 eq) and N,N-diisopropylethylamine (3.91 g, 30.25 mmol, 5.27 mL, 2 eq) in tetrahydrofuran (100 mL) was added a solution of methyl 3-(bromomethyl)-6-chloropicolinate (4 g, 15.12 mmol, 1 eq) in tetrahydrofuran (30 mL) dropwise at 40° C. The reaction mixture was then stirred at 60° C. for 12 h. The mixture was concentrated to give a residue. The residue was purified by silica gel column chromatography (dichloromethane/ethyl acetate=10/1 to 5/1) to afford tert-butyl 4-(2-chloro-7-oxo-5H-pyrrolo[3,4-b]pyridin-6(7H)-yl)piperidine-1-carboxylate (4.5 g, 12.79 mmol, 84% yield) as a white solid. MS (ESI) m/z: 296.1 [M-tBu]⁺. ¹H NMR (400 MHz, CDCl₃) δ: 7.81 (d, J=8.0 Hz, 1H) 7.44 (d, J=8.0 Hz, 1H) 4.47 (m, 1H) 4.34 (s, 2H) 4.15-4.30 (m, 2H) 2.84 (m, 2H) 1.83 (m, 2H) 1.64 (m, 2H) 1.45 (s, 9H).

### Step 5:

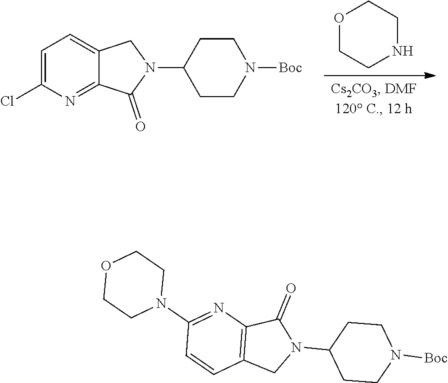 [SMILES: CC(C)(C)OC(=O)N1CCC(N2Cc3ccc(Cl)nc3C2=O)CC1 ; C1COCCN1 ; CC(C)(C)OC(=O)N1CCC(N2Cc3ccc(N4CCOCC4)nc3C2=O)CC1]

To a solution of tert-butyl 4-(2-chloro-7-oxo-5H-pyrrolo[3,4-b]pyridin-6-yl)piperidine-1-carboxylate (1 g, 2.84 mmol, 1 eq) in N,N-dimethylformamide (10 mL) was added cesium carbonate (2.78 g, 8.53 mmol, 3 eq) and morpholine (4.95 g, 56.8 mmol, 5 mL, 20 eq). The mixture was stirred at 120° C. for 12 h. The mixture was poured into ice-water (60 mL) and the aqueous phase was extracted with ethyl acetate (40 mL×3). The combined organic phase was washed with brine (20 mL×3), dried with anhydrous sodium sulphate, filtered, and concentrated under vacuum. The residue was purified by silica gel column chromatography (petroleum ether/ethyl acetate=1/1 to 0/1) to afford tert-butyl 4-(2-morpholino-7-oxo-5H-pyrrolo[3,4-b]pyridin-6-yl)piperidine-1-carboxylate (700 mg, 61% yield), obtained as a white solid. MS (ESI) m/z: 403.1 [M+H]⁺.

### Step 6:

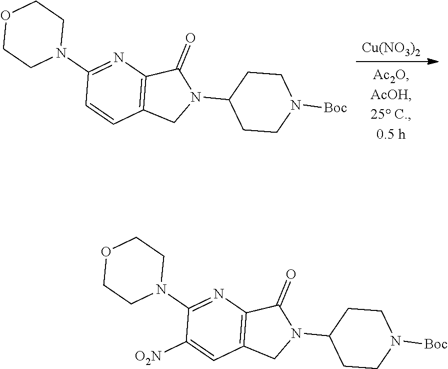 [SMILES: CC(C)(C)OC(=O)N1CCC(N2Cc3ccc(N4CCOCC4)nc3C2=O)CC1 ; "Cu(NO3)2" ; CC(C)(C)OC(=O)N1CCC(N2Cc3cc([N+](=O)[O-])c(N4CCOCC4)nc3C2=O)CC1]

To a solution of tert-butyl 4-(2-morpholino-7-oxo-5H-pyrrolo[3,4-b]pyridin-6-yl)piperidine-1-carboxylate (700 mg, 1.74 mmol, 1 eq) in acetic acid (4 mL) and acetic anhydride (8 mL) was added copper(II) nitrate (420 mg, 1.74 mmol, 1 eq). The mixture was stirred at 25° C. for 0.5 h. The reaction mixture was partitioned between water (200 mL) and ethyl acetate (300 mL). The organic phase was separated, washed with an aqueous solution of sodium bicarbonate (100 mL×3), dried over sodium sulfate, filtered, and concentrated under reduced pressure to give a residue. The residue was purified by column chromatography (silica gel, eluted petroleum ether/ethyl acetate=1/1 to 0/1) to afford tert-butyl 4-(2-morpholino-3-nitro-7-oxo-5H-pyrrolo[3,4-b]pyridin-6-yl)piperidine-1-carboxylate (700 mg, 90% yield) as a yellow solid. MS (ESI) m/z: 392.3 [M-tBu]⁺.

### Step 7:

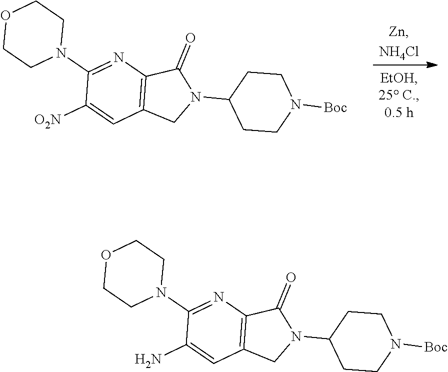

To a solution of tert-butyl 4-(2-morpholino-3-nitro-7-oxo-5H-pyrrolo[3,4-b]pyridin-6-yl)piperidine-1-carboxylate (700 mg, 1.56 mmol, 1 eq) in ethyl alcohol (10 mL) was added ammonium chloride (837 mg, 15.6 mmol, 10 eq) and zinc (1.02 g, 15.64 mmol, 10 eq). The mixture was stirred at 25° C. for 0.5 h. The reaction mixture was filtered, and the filtrate was concentrated. The residue was purified by preparative thin layer chromatography (silica gel, dichloromethane:methanol=20:1) to afford tert-butyl 4-(3-amino-2-morpholino-7-oxo-5H-pyrrolo[3,4-b]pyridin-6-yl)piperidine-1-carboxylate (300 mg, 718.56 μmol, 45% yield), as a red solid. MS (ESI) m/z: 418.4 [M+H]⁺.

### Step 8:

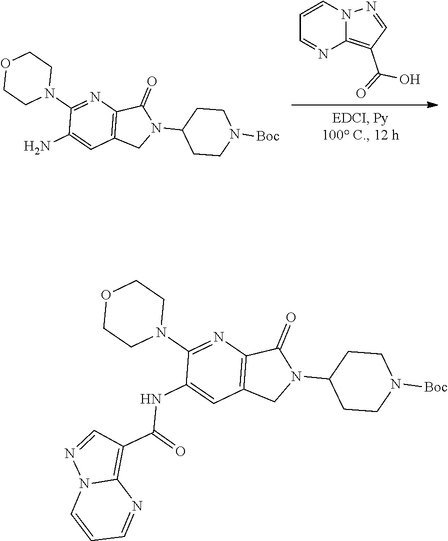 [SMILES: CC(C)(C)OC(=O)N1CCC(N2Cc3cc(N)c(N4CCOCC4)nc3C2=O)CC1 ; O=C(O)c1cnn2cccnc12 ; CC(C)(C)OC(=O)N1CCC(N2Cc3cc(NC(=O)c4cnn5cccnc45)c(N4CCOCC4)nc3C2=O)CC1]

To a mixture of tert-butyl 4-(3-amino-2-morpholino-7-oxo-5H-pyrrolo[3,4-b]pyridin-6(7H)-yl)piperidine-1-carboxylate (0.3 g, 718.56 μmol, 1 eq) and pyrazolo[1,5-a]pyrimidine-3-carboxylic acid (0.13 g, 796.89 μmol, 1.11 eq) in pyridine (8 mL) was added 1-(3-dimethylaminopropyl)-3-ethylcarbodiimide hydrochloride (0.7 g, 3.65 mmol, 5.08 eq) and stirred at 100° C. for 12 h. The mixture was concentrated to give a residue. The residue was purified by preparative thin-layer chromatography (dichloromethane/methyl alcohol=10/1) to give a crude product. The crude product was purified by semi-preparative reverse phase HPLC (column: Shim-pack C18 150×25×10 m; mobile phase: [water(0.225% formic acid)-acetonitrile]; B %: 40%-58%, 9 min) to afford tert-butyl 4-(2-morpholino-7-oxo-3-(pyrazolo[1,5-a]pyrimidine-3-carboxamido)-5H-pyrrolo[3,4-b]pyridin-6(7H)-yl)piperidine-1-carboxylate (60 mg, 106.64 μmol, 14% yield), obtained as a light yellow solid. MS (ESI) m/z: 563.4 [M+H]⁺.

### Step 9:

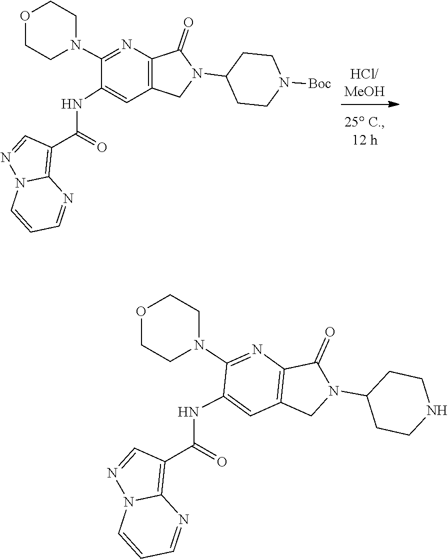 [SMILES: CC(C)(C)OC(=O)N1CCC(N2Cc3cc(NC(=O)c4cnn5cccnc45)c(N4CCOCC4)nc3C2=O)CC1 ; O=C(Nc1cc2c(nc1N1CCOCC1)C(=O)N(C1CCNCC1)C2)c1cnn2cccnc12]

To a solution of tert-butyl 4-(2-morpholino-7-oxo-3-(pyrazolo[1,5-a]pyrimidine-3-carboxamido)-5H-pyrrolo[3,4-b]pyridin-6(7H)-yl)piperidine-1-carboxylate (40 mg, 0.07 mmol, 1 eq) in methanol (5 mL) was added a solution of hydrochloric acid in methanol (4 M, 10 mL, 563 eq). The mixture was stirred at 25° C. for 0.5 h. The reaction mixture was concentrated in vacuo to give N-(2-morpholino-7-oxo-6-(piperidin-4-yl)-6,7-dihydro-5H-pyrrolo[3,4-b]pyridin-3-yl)pyrazolo[1,5-a]pyrimidine-3-carboxamide (30 mg, crude) obtained as a white solid. MS (ESI) m/z: 463.2 [M+H]⁺.

### Step 10:

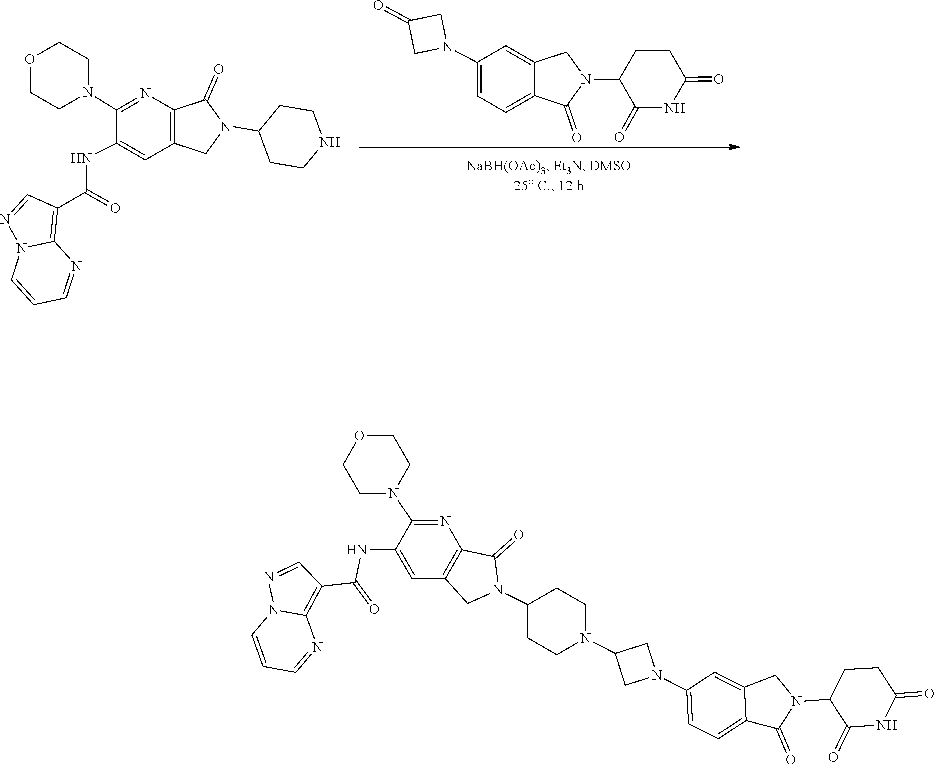 [SMILES: O=C(Nc1cc2c(nc1N1CCOCC1)C(=O)N(C1CCNCC1)C2)c1cnn2cccnc12 ; O=C1CN(c2ccc3c(c2)CN(C2CCC(=O)NC2=O)C3=O)C1 ; O=C1CCC(N2Cc3cc(N4CC(N5CCC(N6Cc7cc(NC(=O)c8cnn9cccnc89)c(N8CCOCC8)nc7C6=O)CC5)C4)ccc3C2=O)C(=O)N1]

A solution of N-[2-morpholino-7-oxo-6-(4-piperidyl)-5H-pyrrolo[3,4-b]pyridin-3-yl]pyrazolo[1,5-a]pyrimidine-3-carboxamide (150 mg, 0.32 mmol, 1 eq), triethylamine (164 mg, 1.62 mmol, 5 eq), and 3-[1-oxo-5-(3-oxoazetidin-1-yl)isoindolin-2-yl]piperidine-2,6-dione (203 mg, 0.65 mmol, 2 eq) in dimethyl sulfoxide (4 mL) was stirred at 50° C. for 4 h. Sodium cyanoborohydride (275 mg, 1.30 mmol, 4 eq) was then added to the mixture at 25° C. and was stirred at 25° C. for 12 h. The reaction mixture was quenched by the addition of water (10 mL), and then diluted with water (10 mL) and extracted with dichloromethane (50 mL×3). The combined organic layers were washed with brine (50 mL×3), dried over sodium sulfate, filtered, and concentrated under reduced pressure to give a residue. The residue was purified by preparative thin-layer chromatography (dichloromethane:methanol=10:1) followed by purification by semi-preparative reverse phase HPLC (Shim-pack C18 150×25×10 m; mobile phase: [water (0.225% formic acid)-acetonitrile]; B %: 9%-31%, 11 min) to afford N-[6-[1-[1-[2-(2,6-dioxo-3-piperidyl)-1-oxo-isoindolin-5-yl]azetidin-3-yl]-4-piperidyl]-2-morpholino-7-oxo-5H-pyrrolo[3,4-b]pyridin-3-yl]pyrazolo[1,5-a]pyrimidine-3-carboxamide (36.7 mg, 0.047 mmol, 14% yield, 98% purity) as a white solid. MS (ESI) m/z: 760.2 [M+H]⁺. ¹H NMR (400 MHz, DMSO-d₆) δ=11.00-10.86 (m, 1H), 10.58 (s, 1H), 9.42 (dd, J=1.6, 7.2 Hz, 1H), 9.08-8.95 (m, 2H), 8.77 (s, 1H), 7.50 (d, J=8.4 Hz, 1H), 7.40 (dd, J=4.0, 7.2 Hz, 1H), 6.59-6.43 (m, 2H), 5.04 (dd, J=5.2, 13.2 Hz, 1H), 4.45 (s, 2H), 4.37-4.26 (m, 1H), 4.23-4.14 (m, 1H), 4.10-3.98 (m, 3H), 3.95-3.86 (m, 4H), 3.71 (t, J=6.0 Hz, 2H), 3.11-3.03 (m, 4H), 2.99-2.82 (m, 3H), 2.63-2.53 (m, 2H), 2.40-2.31 (m, 1H), 2.11-1.92 (m, 3H), 1.88-1.67 (m, 4H).

TABLE 12
The following compounds may be prepared in an analogous fashion as described for the
synthesis of Example 404 and the procedures described in Schemes 1-7.

| | | ¹H NMR |
|---|---|---|
| | | All spectra acquired at 400 MHz unless |
| Example | MS | otherwise indicated |
| Example 404 | See Above | |
| 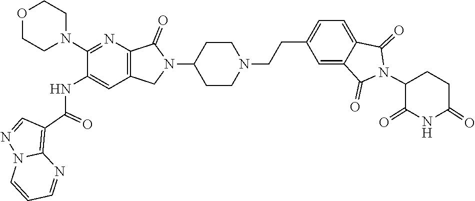 [SMILES: O=C1CCC(N2C(=O)c3ccc(CCN4CCC(N5Cc6cc(NC(=O)c7cnn8cccnc78)c(N7CCOCC7)nc6C5=O)CC4)cc3C2=O)C(=O)N1] | 747.3 | DMSO-d₆ δ = 11.13 (s, 1 H), 10.60 (s, 1 H), 9.61-9.81 (m, 1 H), 9.44 (d, J = 6.8 Hz, 1 H), 9.05 (d, J = 3.6 Hz, 2 H), 8.78 (s, 1 H), 7.77-8.00 (m, 3 H), 7.38-7.45 (m, 1 H), 5.16 (d, J = 8.0 Hz, 1 H), 4.47 (s, 2 H), 3.91 (s, 4 H), 3.70 (s, 1 H), 3.44 (s, 1 H), 3.08 (s, 4 H), 2.88 (d, J = 13.2 Hz, 1 H), 2.55-2.70 (m, 3 H), 1.91-2.24 (m, 6 H), 1.74 (s, 2 H), 1.23 (s, 1 H), 0.81-0.87 (m, 2 H). |
| Example 405 | | |
| 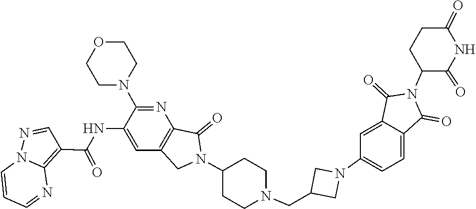 [SMILES: O=C1CCC(N2C(=O)c3ccc(N4CC(CN5CCC(N6Cc7cc(NC(=O)c8cnn9cccnc89)c(N8CCOCC8)nc7C6=O)CC5)C4)cc3C2=O)C(=O)N1] | 788.4 | DMSO-d₆ δ = 10.57 (s, 1 H) 9.12 (s, 1 H) 8.81-8.91 (m, 3 H) 8.23 (br s, 1 H) 7.66 (d, J = 8.0 Hz, 1 H) 7.16 (dd, J = 6.8, 4.4 Hz, 1 H) 6.80 (s, 1 H) 6.53 (br d, J = 8.4 Hz, 1 H) 4.95 (br dd, J = 12.0, 5.2 Hz, 1 H) 4.36 (br s, 2 H) 4.21 (br s, 2 H) 3.97-4.03 (m, 4 H) 3.69-3.84 (m, 3 H) 3.50 (s, 2 H) 3.16-3.34 (m, 5 H) 2.69-3.00 (m, 6 H) 2.10-2.17 (m, 1 H) 1.76-2.03 (m, 4 H) 1.24-1.29 (m, 1 H) |
| Example 406 | | |
| 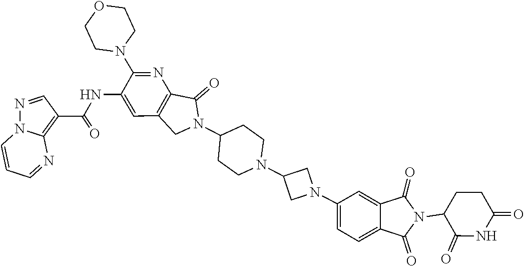 [SMILES: O=C1CCC(N2C(=O)c3ccc(N4CC(N5CCC(N6Cc7cc(NC(=O)c8cnn9cccnc89)c(N8CCOCC8)nc7C6=O)CC5)C4)cc3C2=O)C(=O)N1] | 788.4 | DMSO-d₆ δ = 11.0-11.2 (m, 1H), 10.60 (s, 1H), 9.43 (dd, 1H, J = 1.4, 6.9 Hz), 9.0-9.1 (m, 2H), 8.78 (s, 1H), 7.66 (d, 1H, J = 8.2 Hz), 7.40 (dd, 1H, J = 4.3, 6.9 Hz), 6.8-6.8 (m, 1H), 6.67 (dd, 1H, J = 1.9, 8.3 Hz), 5.06 (dd, 1H, J = 5.1, 12.8 Hz), 4.47 (s, 2H), 4.0- 4.2 (m, 3H), 3.8-3.9 (m, 5H), 3.0-3.1 (m, 4H), 2.9-3.0 (m, 2H), 2.8-2.9 (m, 1H), 2.6- 2.6 (m, 2H), 2.54 (s, 4H), 1.9-2.1 (m, 2H), 1.7-1.9 (m, 3H) |
| Example 407 | | |

### Preparation of Example 408: N-[6-(difluoromethyl)-2-[1-[[1-[2-(2,6-dioxo-3-piperidyl)-1,3-dioxo-isoindolin-5-yl]-3-fluoro-azetidin-3-yl]methyl]-4-piperidyl]indazol-5-yl]pyrazolo[1,5-a]pyrimidine-3-carboxamide

### Step 1:

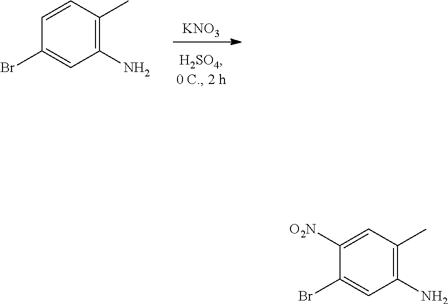

To a solution of 5-bromo-2-methyl-aniline (5 g, 26.87 mmol, 1 eq) in sulfuric acid (40 mL) was added potassium nitrate (2.86 g, 28.29 mmol, 1.05 eq) at 0° C. The reaction mixture was stirred at 0° C. for 2 h. The reaction mixture was poured on crushed ice and stirred at 25° C. for 30 min. Then the separated solid was filtered and washed with water. The residue was purified by silica gel chromatography (ISCO®; 40 g SepaFlash® Silica Flash Column, eluent of 0 to 50% ethyl acetate in petroleum ether gradient, 40 mL/min). The desired compound, 5-bromo-2-methyl-4-nitro-aniline (4 g, 17.31 mmol, 64% yield), was obtained as a light yellow solid. ¹H NMR (400 MHz, CDCl₃) δ=7.88 (s, 1H), 6.92 (s, 1H), 2.17 (s, 3H).

### Step 2:

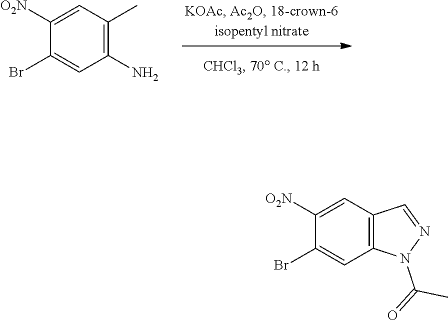 [SMILES: Cc1cc([N+](=O)[O-])c(Br)cc1N ; CC(=O)n1ncc2cc([N+](=O)[O-])c(Br)cc21]

To a solution of 5-bromo-2-methyl-4-nitro-aniline (500 mg, 2.16 mmol, 1 eq) in chloroform (8 mL) was added acetyl acetate (887 mg, 8.69 mmol, 4.02 eq) at 0° C., followed by potassium acetate (419 mg, 4.27 mmol, 1.97 eq), 18-crown-6 (177 mg, 0.67 mmol, 0.31 eq), and isopentyl nitrite (516 mg, 4.41 mmol, 2.04 eq). The reaction mixture was stirred at 70° C. for 12 h. The reaction mixture was quenched by the addition of water (10 mL), and then diluted with water (30 mL) and extracted with dichloromethane (30 mL×3). The combined organic layers were washed with brine (50 mL×3), dried over anhydrous sodium sulfate, filtered, and concentrated under reduced pressure to give a residue. The residue was purified by silica gel chromatography (ISCO®; 12 g SepaFlash® Silica Flash Column, Eluent of 0 to 40% ethyl acetate/petroleum ether gradient, 30 mL/min). The desired compound 1-(6-bromo-5-nitro-indazol-1-yl)ethanone (300 mg, 1.06 mmol, 48% yield) was obtained as a yellow solid. ¹H NMR (400 MHz, CDCl₃) δ=8.91 (s, 1H), 8.29 (s, 1H), 8.24 (s, 1H), 2.83 (s, 3H).

### Step 3:

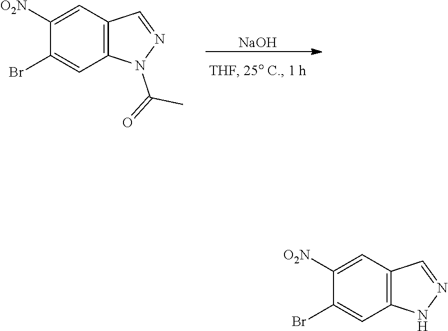 [SMILES: CC(=O)n1ncc2cc([N+](=O)[O-])c(Br)cc21 ; "NaOH" ; O=[N+]([O-])c1cc2cn[nH]c2cc1Br]

To a solution of 1-(6-bromo-5-nitro-indazol-1-yl)ethanone (20 g, 70.41 mmol, 1 eq) in tetrahydrofuran (10 mL) and water (10 mL) was added sodium hydroxide (14.08 g, 352 mmol, 5 eq). The reaction mixture was stirred at 25° C. for 1 h. The reaction mixture was diluted with water (40 mL) and extracted with ethyl acetate (40 mL×3). The combined organic layers were washed with brine (50 mL×3), dried over anhydrous sodium sulfate, filtered, and concentrated under reduced pressure to give a residue. The residue was purified by silica gel chromatography (ISCO®; 80 g SepaFlash® Silica Flash Column, Eluent of 0 to 60% ethyl acetate/petroleum ether gradient, 60 mL/min). The desired compound 6-bromo-5-nitro-2H-indazole (5.4 g, 22.31 mmol, 31% yield) was obtained as a yellow solid. ¹H NMR (400 MHz, DMSO-d₆) δ=13.72 (s, 1H), 8.62 (s, 1H), 8.34 (s, 1H), 8.06 (s, 1H).

### Step 4:

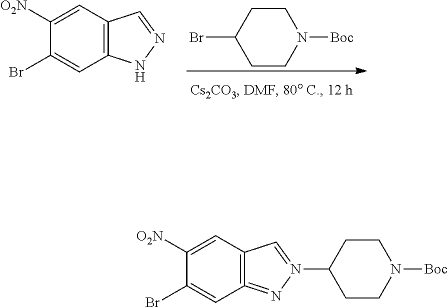

To a solution of 6-bromo-5-nitro-2H-indazole (5.4 g, 22.31 mmol, 1 eq) and tert-butyl 4-bromopiperidine-1-carboxylate (11.79 g, 44.62 mmol, 2 eq) in N,N-dimethylformamide (20 mL) was added cesium carbonate (21.81 g, 66.93 mmol, 3 eq). The reaction mixture was stirred at 80° C. for 12 h. The reaction mixture was diluted with water (40 mL) and extracted with ethyl acetate (40 mL×3). The combined organic layers were washed with brine (50 mL×3), dried over anhydrous sodium sulfate, filtered, and concentrated under reduced pressure to give a residue. The residue was purified by silica gel chromatography (ISCO®; 12 g SepaFlash® Silica Flash Column, Eluent of 0˜40% ethyl acetate/petroleum ether gradient, 30 mL/min). The desired compound tert-butyl 4-(6-bromo-5-nitro-indazol-2-yl)piperidine-1-carboxylate (2.8 g, 6.58 mmol, 29% yield) was obtained as a light yellow solid. MS (ESI) m/z: 369.2 [M−56]⁺. ¹H NMR (400 MHz, DMSO-d₆) δ=8.82 (s, 1H), 8.62 (s, 1H), 8.19 (s, 1H), 4.89-4.75 (m, 1H), 4.10 (d, J=12.0 Hz, 2H), 2.96 (s, 2H), 2.13 (d, J=11.2 Hz, 2H), 2.02-1.85 (m, 2H), 1.43 (s, 9H).

### Step 5:

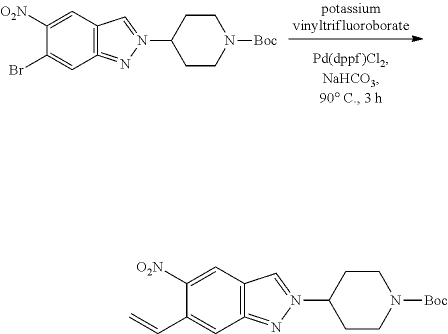

To a solution of tert-butyl 4-(6-bromo-5-nitro-indazol-2-yl)piperidine-1-carboxylate (2.8 g, 6.58 mmol, 1 eq) and potassium vinyltrifluoroborate (1.06 g, 7.90 mmol, 1.2 eq) in dioxane (25 mL) and water (2.5 mL) was added sodium bicarbonate (1.66 g, 19.75 mmol, 3 eq) and Pd(dppf)Cl₂ (1.61 g, 1.98 mmol, 0.3 eq). The reaction mixture was stirred at 90° C. for 3 h under a nitrogen atmosphere. The reaction mixture was quenched diluted with water (40 mL) and extracted with ethyl acetate (40 mL×3). The combined organic layers were washed with brine (50 mL×3), dried over anhydrous sodium sulfate, filtered, and concentrated under reduced pressure to give a residue. The residue was purified by silica gel chromatography (ISCO®; 12 g SepaFlash® Silica Flash Column, Eluent of 0 to 40% ethyl acetate/petroleum ether gradient @30 mL/min). The desired compound tert-butyl 4-(5-nitro-6-vinyl-indazol-2-yl)piperidine-1-carboxylate (2.4 g, 6.44 mmol, 97% yield) was obtained as a yellow solid. MS (ESI) m/z: 317.2 [M−56]⁺. ¹H NMR (400 MHz, CDCl₃) δ=8.47 (s, 1H), 8.17 (s, 1H), 7.84 (s, 1H), 7.18 (dd, J=10.8, 17.2 Hz, 1H), 5.71 (d, J 17.2 Hz, 1H), 5.41 (d, J=10.8 Hz, 1H), 4.66-4.58 (m, 1H), 4.38-4.27 (m, 2H), 2.97 (t, J=12.4 Hz, 2H), 2.27 (d, J=12.4 Hz, 2H), 2.18-2.08 (m, 2H), 1.50 (s, 9H).

### Step 6:

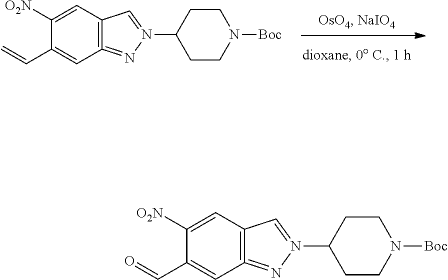

A mixture of tert-butyl 4-(5-nitro-6-vinyl-indazol-2-yl)piperidine-1-carboxylate (200 mg, 0.54 mmol, 1 eq), osmium tetroxide (0.5 g, 1.97 mmol, 3.66 eq), sodium periodate (482 mg, 2.26 mmol, 4.2 eq) and 2,6-dimethylpyridine (121 mg, 1.13 mmol, 2.1 eq) in a mixture of dioxane (3 mL) and water (3 mL) stirred at 0° C. for 1 h. The reaction mixture was quenched with the addition of sodium thiosulfate (10 mL) and extracted with dichloromethane (30 mL×3). The combined organic layers were washed with brine (50 mL×3), dried over anhydrous sodium sulfate, filtered, and concentrated under reduced pressure to give a residue. The residue was purified by preparative Thin layer chromatography (petroleum ether:ethyl acetate=1:1) to afford tert-butyl 4-(6-formyl-5-nitro-indazol-2-yl)piperidine-1-carboxylate (120 mg, 0.32 mmol, 59% yield) as a yellow solid. MS (ESI) m/z: 319.1 [M−56]⁺. ¹H NMR: (400 MHz, DMSO-d₆) δ=10.20 (s, 1H), 8.93 (s, 1H), 8.72 (s, 1H), 8.21 (s, 1H), 4.89 (t, J=11.2 Hz, 1H), 4.19-4.04 (m, 2H), 3.06-2.87 (m, 2H), 2.16 (d, J=12.0 Hz, 2H), 2.02-1.93 (m, 2H), 1.43 (s, 9H).

### Step 7:

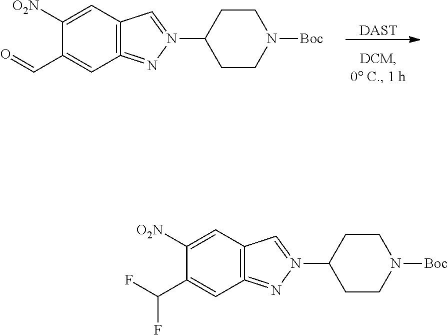

To a solution of tert-butyl 4-(6-formyl-5-nitro-indazol-2-yl)piperidine-1-carboxylate (500 mg, 1.34 mmol, 1 eq) in dichloromethane (5 mL) was added diethylamine sulfur trifluoride (646 mg, 4.01 mmol, 3 eq) at 0° C. The reaction mixture was stirred at 0° C. for 1 h. The reaction mixture was poured into ice-cooled saturated sodium bicarbonate solution (40 mL), and then diluted with water (20 mL) and extracted with dichloromethane (30 mL×3). The combined organic layers were washed with brine (40 mL×3), dried over anhydrous sodium sulfate, filtered, and concentrated under reduced pressure to give a residue. The residue was purified by silica gel chromatography (ISCO®; 12 g SepaFlash® Silica Flash Column, Eluent of 0 to 30% ethyl acetate/petroleum ether gradient, 30 mL/min). The desired compound tert-butyl 4-[6-(difluoromethyl)-5-nitro-indazol-2-yl]piperidine-1-carboxylate (380 mg, 0.96 mmol, 71% yield) was obtained as a light yellow solid. MS (ESI) m/z: 341.1 [M−56]⁺. ¹H NMR (400 MHz, DMSO-d₆) δ=8.95 (s, 1H), 8.87 (s, 1H), 8.09 (s, 1H), 7.75-7.26 (m, 1H), 4.99-4.81 (m, 1H), 4.12 (d, J=11.6 Hz, 2H), 3.09-2.91 (m, 2H), 2.16 (d, J=10.0 Hz, 2H), 2.06-1.89 (m, 2H), 1.43 (s, 9H).

### Step 8:

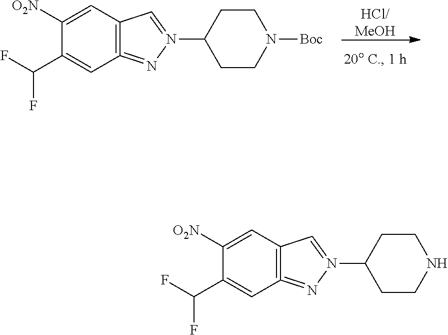

To a solution of tert-butyl 4-[6-(difluoromethyl)-5-nitro-indazol-2-yl]piperidine-1-carboxylate (1.4 g, 3.53 mmol, 1 eq) in dichloromethane (5 mL) was added hydrochloride/methanol (4 M, 10 mL, 11.33 eq). The reaction mixture was stirred at 20° C. for 0.5 h. The reaction mixture was concentrated under reduced pressure to give a residue. The residue was resuspended in a 10:1 solution of dichloromethane:methanol=10:1 (20 mL), and ammonium hydroxide was added to adjust the pH to 8-9. The mixture was then concentrated under reduced pressure to give a residue. The residue was purified by column chromatography (dichloromethane:methanol=1:0 to 10:1). The desired compound 6-(difluoromethyl)-5-nitro-2-(4-piperidyl)indazole (1.2 g, crude) was obtained as a light yellow solid. MS (ESI) m/z: 296.8 [M+H]⁺.

### Step 9:

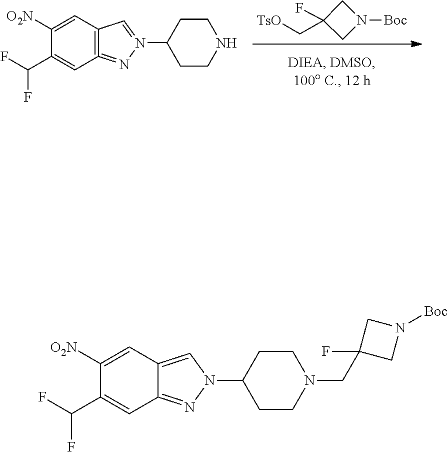

To a stirred solution of 6-(difluoromethyl)-5-nitro-2-(4-piperidyl)indazole (1 g, 3.38 mmol, 1 eq) and tert-butyl 3-fluoro-3-(ptolylsulfonyloxymethyl)azetidine-1-carboxylate (2.18 g, 6.08 mmol, 1.8 eq) in dimethyl sulfoxide (10 mL) was added N,N-diisopropylethylamine (2.18 g, 16.88 mmol, 5 eq). The reaction mixture was heated to 100° C. for 12 h. The reaction mixture was quenched by the addition of water (10 mL), and then diluted with water (20 mL) and extracted with ethyl acetate (40 mL×3). The combined organic layers were washed with brine (60 mL×3), dried over anhydrous sodium sulfate, filtered, and concentrated under reduced pressure to give a residue. The residue was purified by silica gel chromatography (ISCO®; 12 g SepaFlash® Silica Flash Column, Eluent of 0 to 50% ethyl acetate/petroleum ether gradient, 30 mL/min). The desired compound tert-butyl 3-[[4-[6-(difluoromethyl)-5-nitro-indazol-2-yl]-1-piperidyl]methyl]-3-fluoro-azetidine-1-carboxylate (1.2 g, 2.48 mmol, 73% yield) was obtained as a light yellow solid. ¹H NMR (400 MHz, CDCl₃) δ=8.68 (s, 1H), 8.28 (s, 1H), 8.19 (s, 1H), 7.68-7.33 (m, 1H), 4.58-4.41 (m, 1H), 4.08-3.99 (m, 4H), 3.18-3.08 (m, 2H), 2.93-2.81 (m, 2H), 2.54-2.42 (m, 2H), 2.29-2.22 (m, 4H), 1.47 (s, 9H).

### Step 10:

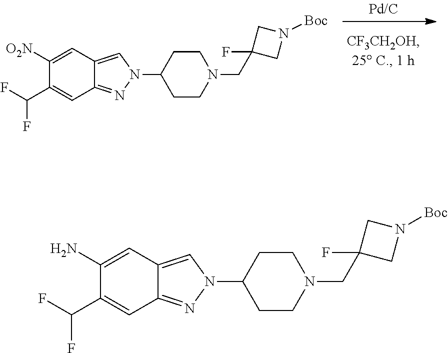 [SMILES: CC(C)(C)OC(=O)N1CC(F)(CN2CCC(n3cc4cc([N+](=O)[O-])c(C(F)F)cc4n3)CC2)C1 ; CC(C)(C)OC(=O)N1CC(F)(CN2CCC(n3cc4cc(N)c(C(F)F)cc4n3)CC2)C1]

To a solution of tert-butyl 3-[[4-[6-(difluoromethyl)-5-nitro-indazol-2-yl]-1-piperidyl]methyl]-3-fluoro-azetidine-1-carboxylate (1.5 g, 3.10 mmol, 1 eq) in trifluoroethanol (10 mL) was added palladium on carbon (200 mg, 10% purity). The reaction mixture was stirred at 25° C. for 1 h with hydrogen (15 psi). The reaction mixture was filtered and concentrated under reduced pressure to give a residue. The residue was purified by silica gel chromatography (ISCO®; 12 g SepaFlash® Silica Flash Column, Eluent of 0 to 80% ethyl acetate/petroleum ether gradient, mL/min) to afford tert-butyl 3-[[4-[5-amino-6-(difluoromethyl)indazol-2-yl]-1-piperidyl]methyl]-3-fluoro-azetidine-1-carboxylate (1 g, 2.21 mmol, 71% yield) as a light yellow solid. MS (ESI) m/z: 454.0 [M+H]⁺.

### Step 11:

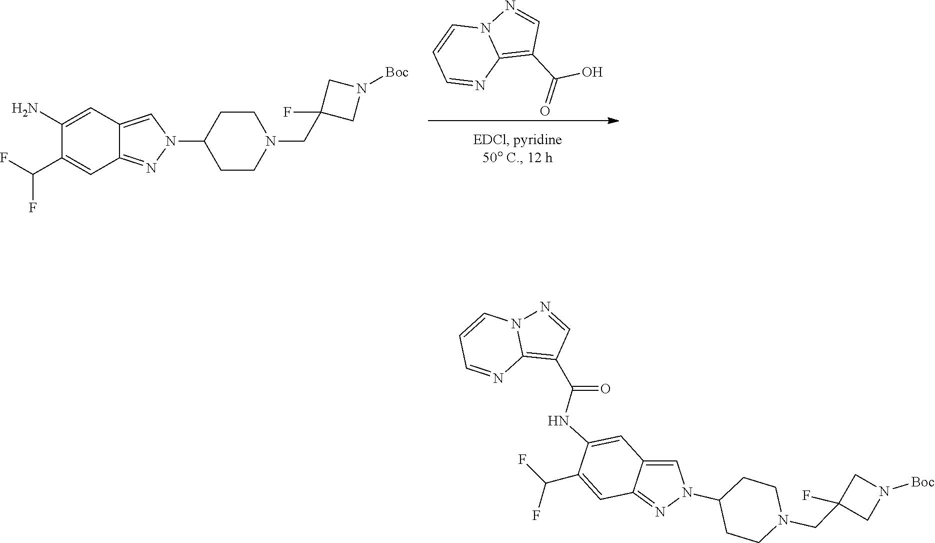 [SMILES: CC(C)(C)OC(=O)N1CC(F)(CN2CCC(n3cc4cc(N)c(C(F)F)cc4n3)CC2)C1 ; O=C(O)c1cnn2cccnc12 ; CC(C)(C)OC(=O)N1CC(F)(CN2CCC(n3cc4cc(NC(=O)c5cnn6cccnc56)c(C(F)F)cc4n3)CC2)C1]

To a solution of pyrazolo[1,5-a]pyrimidine-3-carboxylic acid (79 mg, 0.49 mmol, 1.1 eq) in pyridine (5 mL) was added tert-butyl 3-[[4-[5-amino-6-(difluoromethyl)indazol-2-yl]-1-piperidyl]methyl]-3-fluoro-azetidine-1-carboxylate (200 mg, 0.44 mmol, 1 eq) and 1-(3-dimethylaminopropyl)-3-ethylcarbodiimide hydrochloride (423 mg, 2.21 mmol, 5 eq). The reaction mixture was stirred at 50° C. for 12 h. The reaction mixture was concentrated under reduced pressure to give a residue. The residue was purified by silica gel chromatography (ISCO®; 12 g SepaFlash® Silica Flash Column, Eluent of 0 to 100% ethyl acetate/petroleum ether to 100 to 90% dichloromethane:methanol gradient, 30 m/min). The desired compound tert-butyl 3-[[4-[6-(difluoromethyl)-5-(pyrazolo[1,5-a]pyrimidine-3-carbonylamino)indazol-2-yl]-1-piperidyl]methyl]-3-fluoro-azetidine-1-carboxylate (180 mg, 0.3 mmol, 68% yield) was obtained as a light yellow solid. MS (ESI) m/z: 599.3 [M+H]⁺. ¹H NMR (400 MHz, CDCl₃) δ=9.97 (s, 1H), 8.84 (d, J=7.0 Hz, 1H), 8.79 (s, 1H), 8.75 (d, J=4.0 Hz, 1H), 8.46 (s, 1H), 8.07-7.92 (m, 2H), 7.11-6.76 (m, 2H), 4.51-4.37 (m, 1H), 4.10-3.96 (m, 4H), 3.17-3.07 (m, 2H), 2.94-2.79 (m, 2H), 2.52-2.41 (m, 2H), 2.30-2.20 (m, 4H), 1.47 (s, 9H).

### Step 12:

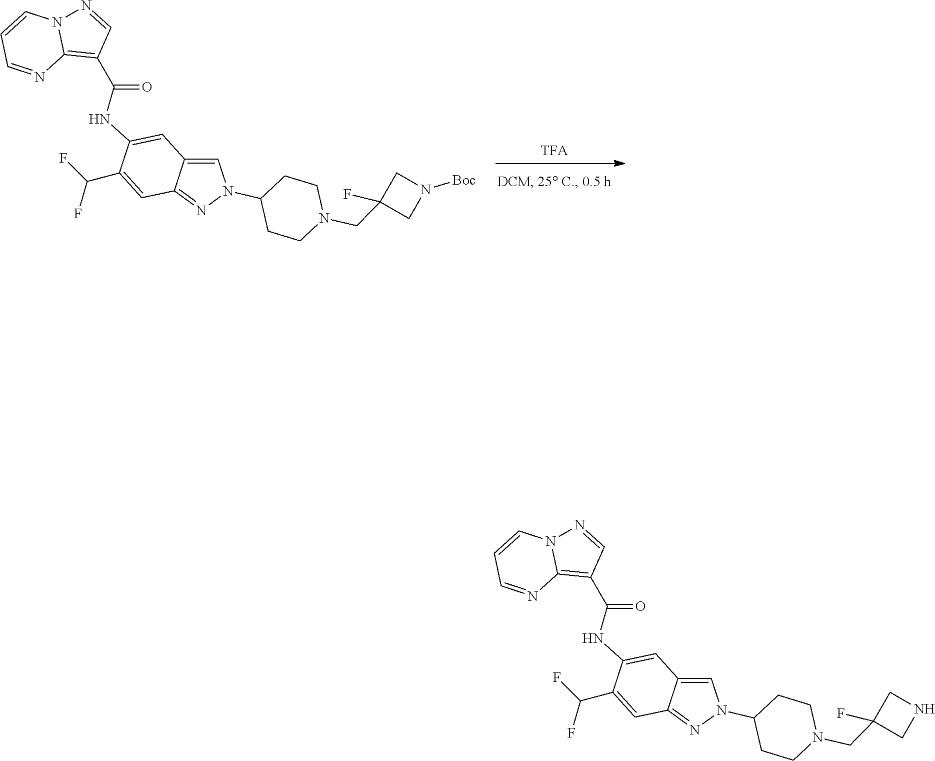 [SMILES: CC(C)(C)OC(=O)N1CC(F)(CN2CCC(n3cc4cc(NC(=O)c5cnn6cccnc56)c(C(F)F)cc4n3)CC2)C1 ; O=C(Nc1cc2cn(C3CCN(CC4(F)CNC4)CC3)nc2cc1C(F)F)c1cnn2cccnc12]

To a solution of tert-butyl 3-[[4-[6-(difluoromethyl)-5-(pyrazolo[1,5-a]pyrimidine-3-carbonylamino)indazol-2-yl]-1-piperidyl]methyl]-3-fluoro-azetidine-1-carboxylate (180 mg, 0.3 mmol, 1 eq) in dichloromethane (2 mL) was added trifluoroacetic acid (3.08 g, 27.01 mmol, 2 mL, 89.83 eq). The reaction mixture was stirred at 25° C. for 0.5 h. The reaction mixture was concentrated under reduced pressure to give a residue. The residue was resuspended in a 10:1 solution of dichloromethane:methanol=10:1 (20 mL), and ammonium hydroxide was added to adjust the pH=8-9. The mixture was concentrated to provide N-[6-(difluoromethyl)-2-[1-[(3-fluoroazetidin-3-yl)methyl]-4-piperidyl]indazol-5-yl]pyrazolo[1,5-a]pyrimidine-3-carboxamide (150 mg, crude) as light yellow solid which was used in subsequent reactions without further purification.

### Step 13:

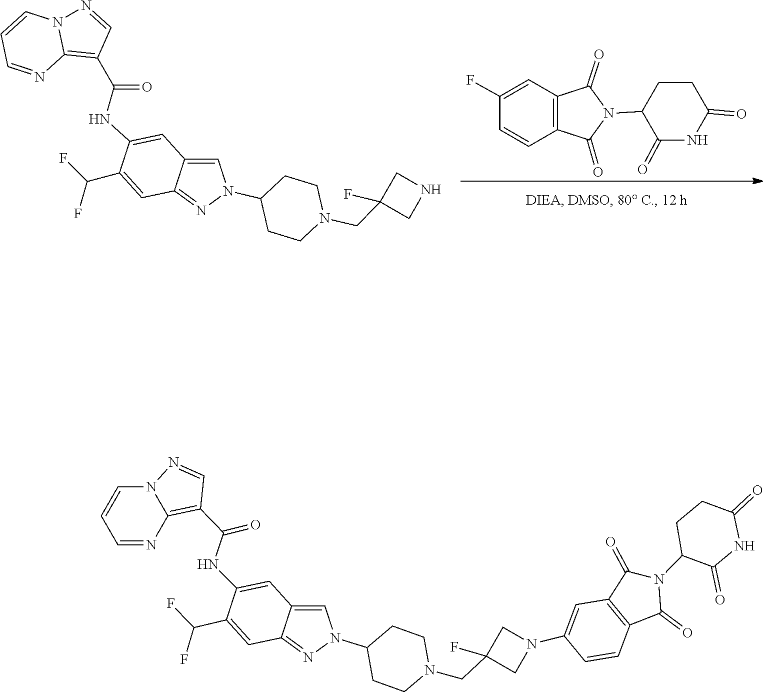 [SMILES: O=C(Nc1cc2cn(C3CCN(CC4(F)CNC4)CC3)nc2cc1C(F)F)c1cnn2cccnc12 ; O=C1CCC(N2C(=O)c3ccc(F)cc3C2=O)C(=O)N1 ; O=C1CCC(N2C(=O)c3ccc(N4CC(F)(CN5CCC(n6cc7cc(NC(=O)c8cnn9cccnc89)c(C(F)F)cc7n6)CC5)C4)cc3C2=O)C(=O)N1]

To a solution of N-[6-(difluoromethyl)-2-[1-[(3-fluoroazetidin-3-yl)methyl]-4-piperidyl]indazol-5-yl]pyrazolo[1,5-a]pyrimidine-3-carboxamide (150 mg, 0.3 mmol, 1 eq) and 2-(2,6-dioxo-3-piperidyl)-5-fluoro-isoindoline-1,3-dione (166 mg, 0.6 mmol, 2 eq) in dimethyl sulfoxide (3 mL) was added N,N-diisopropylethylamine (389 mg, 3.01 mmol, 10 eq). The reaction mixture was stirred at 80° C. for 12 h. The reaction mixture was quenched by addition water (10 mL), and then diluted with water (30 mL) and extracted with dichloromethane (30 mL×3). The combined organic layers were washed with brine (60 mL×3), dried over anhydrous sodium sulfate, filtered, and concentrated under reduced pressure to give a residue. The residue was purified by preparative thin-layer chromatography (dichloromethane:methanol=10:1). Then the residue was purified by preparative HPLC (column: Phenomenex Synergi C18 150×25 mm×10 um; mobile phase: [water(0.225% formic acid)-acetonitrile]; B %: 12%-42%, 10 min). The desired compound N-[6-(difluoromethyl)-2-[1-[[1-[2-(2,6-dioxo-3-piperidyl)-1,3-dioxo-isoindolin-5-yl]-3-fluoro-azetidin-3-yl]methyl]-4-piperidyl]indazol-5-yl]pyrazolo[1,5-a]pyrimidine-3-carboxamide (115.0 mg, 0.15 mmol, 49% yield, 98% purity) was obtained as a yellow solid. MS (ESI) m/z: 754.7 [M]. ¹H NMR (400 MHz, DMSO-d₆) δ=11.08 (s, 1H), 9.97 (s, 1H), 9.39 (dd, J=1.6, 7.0 Hz, 1H), 8.88 (dd, J=1.6, 4.0 Hz, 1H), 8.72 (s, 1H), 8.57 (s, 1H), 8.34 (s, 1H), 7.96 (s, 1H), 7.69 (d, J=8.4 Hz, 1H), 7.41-7.06 (m, 2H), 6.92 (d, J=2.0 Hz, 1H), 6.78 (dd, J=2.0, 8.4 Hz, 1H), 5.07 (dd, J=5.6, 12.8 Hz, 1H), 4.64-4.47 (m, 1H), 4.30-4.13 (m, 4H), 3.13-3.05 (m, 2H), 3.02-2.93 (m, 2H), 2.91-2.83 (m, 1H), 2.58-2.54 (m, 2H), 2.45-2.40 (m, 2H), 2.22-2.09 (m, 4H), 2.05-1.98 (m, 1H).

TABLE 13
The following compounds may be prepared in an analogous fashion as described for the
synthesis of Example 408 using instead a substituted pyrazolo[1,5-a]pyrimidine-3-carboxylic acid in step 11.

| | | ¹H NMR |
|---|---|---|
| | | All spectra acquired at 400 MHz unless |
| Example | MS | otherwise indicated |
| Example 408 | See Above | |
| 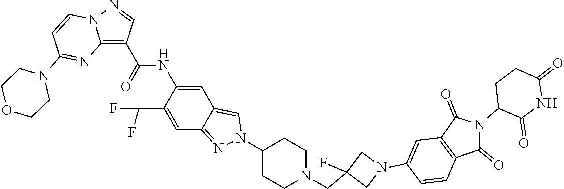 [SMILES: O=C1CCC(N2C(=O)c3ccc(N4CC(F)(CN5CCC(n6cc7cc(NC(=O)c8cnn9ccc(N%10CCOCC%10)nc89)c(C(F)F)cc7n6)CC5)C4)cc3C2=O)C(=O)N1] | 840.1 | DMSO-d₆ δ = 11.08 (s, 1H), 9.33 (s, 1H), 8.84 (d, J = 7.6 Hz, 1H), 8.55 (s, 1H), 8.30 (s, 1H), 8.05 (s, 1H), 7.93 (s, 1H), 7.69 (d, J = 8.4 Hz, 1H), 7.31-6.99 (m, 1H), 6.94- 6.88 (m, 2H), 6.77 (dd, J = 2.0, 8.4 Hz, 1H), 5.07 (dd, J = 5.6, 12.8 Hz, 1H), 4.61- 4.49 (m, 1H), 4.29-4.13 (m, 4H), 3.78- 3.68 (m, 8H), 3.12-3.04 (m, 2H), 3.01- 2.93 (m, 2H), 2.89-2.81 (m, 1H), 2.58- 2.53 (m, 2H), 2.46-2.41 (m, 2H), 2.22- 2.08 (m, 4H), 2.04-1.96 (m, 1H) |
| Example 409 | | |
| 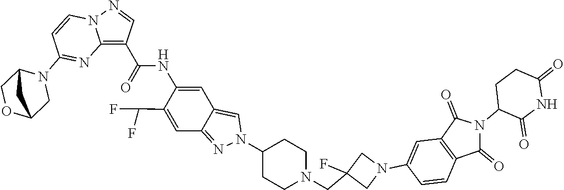 [SMILES: O=C1CCC(N2C(=O)c3ccc(N4CC(F)(CN5CCC(n6cc7cc(NC(=O)c8cnn9ccc(N%10C[C@H]%11C[C@@H]%10CO%11)nc89)c(C(F)F)cc7n6)CC5)C4)cc3C2=O)C(=O)N1] | 852.1 | DMSO-d₆ δ = 11.07 (s, 1H), 9.42 (br s, 1H), 8.80 (dd, J = 2.4, 7.2 Hz, 1H), 8.55 (s, 1H), 8.27 (d, J = 5.2 Hz, 1H), 8.18- 8.03 (m, 1H), 7.93 (s, 1H), 7.69 (d, J = 8.4 Hz, 1H), 7.33-6.98 (m, 1H), 6.92 (d, J = 2.0 Hz, 1H), 6.90-6.42 (m, 2H), 5.12- 5.03 (m, 2H), 4.80-4.68 (m, 1H), 4.61- 4.49 (m, 1H), 4.32-4.10 (m, 5H), 3.84- 3.77 (m, 2H), 3.66-3.42 (m, 2H), 3.08 (br d, J = 11.6 Hz, 2H), 3.03-2.91 (m, 2H), 2.90-2.82 (m, 1H), 2.60 (br d, J = 2.0 Hz, 1H), 2.23-2.07 (m, 5H), 2.06-1.89 (m, 4H) |
| Example 410 | | |

### Preparation of Example 411: N-(2-(1-((1-(6-(2,6-dioxopiperidin-3-yl)-5,7-dioxo-6,7-dihydro-5H-pyrrolo[3,4-b]pyridin-2-yl)-3-fluoroazetidin-3-yl)methyl)piperidin-4-yl)-6-isopropoxy-2H-indazol-5-yl)pyrazolo[1,5-a]pyrimidine-3-carboxamide

Step 1: 6-chloropyridine-2,3-dicarboxylic acid

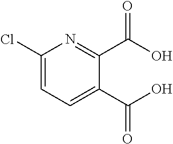 [SMILES: O=C(O)c1ccc(Cl)nc1C(=O)O]

To a solution of sodium hypochlorite (0.6 mol/L, 1000 mL) in carbon tetrachloride (500 mL) was added ruthenium(IV) oxide (500 mg, 3.8 mmol, 71.74 uL) and stirred at 25° C. for 2 h. Then 2-chloroquinoline (7 g, 42.8 mmol, 5.69 mL) was added and the mixture was stirred at 25° C. for 14 h. The aqueous phase was separated, washed with ethyl acetate (3×200 mL) and acidified to pH 2 with 36% concentrated hydrochloric acid, extracted with ethyl acetate (3×200 mL). The combined organic layers were washed with brine (100 mL), dried over anhydrous sodium sulfate and concentrated under reduced pressure to afford 6-chloropyridine-2,3-dicarboxylic acid (6.5 g, 75%) as a white solid, which was used in the next step without further purification. MS (ESI) m/z: 200.0 [M−H]⁻; ¹H NMR (400 MHz, DMSO-d₆) δ 14.66-12.88 (m, 2H), 8.27 (d, J=8.4 Hz, 1H), 7.73 (d, J=8.0 Hz, 1H).

Step 2: 2-chloro-6-(2,6-dioxopiperidin-3-yl)-5H-pyrrolo[3,4-b]pyridine-5,7(6H)-dione

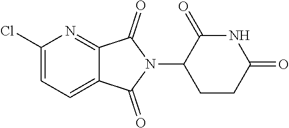 [SMILES: O=C1CCC(N2C(=O)c3ccc(Cl)nc3C2=O)C(=O)N1]

To a solution of 6-chloropyridine-2,3-dicarboxylic acid (3 g, 14.9 mmol) in acetic acid (20 mL) was added sodium acetate (3.78 g, 46.1 mmol) and 3-aminopiperidine-2,6-dione hydrochloride (3.12 g, 19.0 mmol). The mixture was stirred at 100° C. for 12 h. The reaction was concentrated in vacuum. The residue was purified by silica gel column chromatography (petroleum ether/ethyl acetate=6/1 to 0/1) to afford 2-chloro-6-(2,6-dioxopiperidin-3-yl)-5H-pyrrolo[3,4-b]pyridine-5,7(6H)-dione (1 g, 23%) as a white solid. MS (ESI) m/z: 294.0 [M+H]⁺; ¹H NMR (400 MHz, DMSO-d₆) δ 11.17 (s, 1H), 8.42 (d, J=8.4 Hz, 1H), 7.99 (d, J=8.0 Hz, 1H), 5.24 (dd, J=5.2, 12.8 Hz, 1H), 2.96-2.83 (m, 1H), 2.70-2.53 (m, 2H), 2.13-2.02 (m, 1H).

Step 3: N-(2-(1-((1-(6-(2,6-dioxopiperidin-3-yl)-5,7-dioxo-6,7-dihydro-5H-pyrrolo[3,4-b]pyridin-2-yl)-3-fluoroazetidin-3-yl)methyl)piperidin-4-yl)-6-isopropoxy-2H-indazol-5-yl)pyrazolo[1,5-a]pyrimidine-3-carboxamide

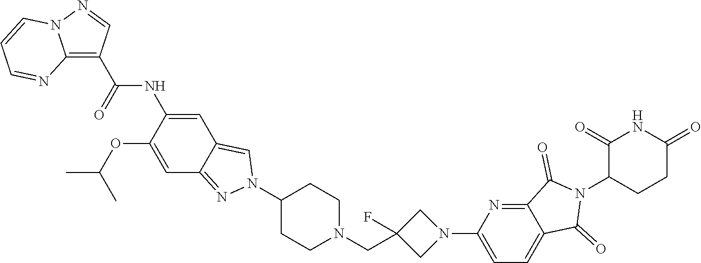 [SMILES: CC(C)Oc1cc2nn(C3CCN(CC4(F)CN(c5ccc6c(n5)C(=O)N(C5CCC(=O)NC5=O)C6=O)C4)CC3)cc2cc1NC(=O)c1cnn2cccnc12]

To a solution of N-(2-(1-((3-fluoroazetidin-3-yl)methyl)piperidin-4-yl)-6-isopropoxy-2H-indazol-5-yl)pyrazolo[1,5-a]pyrimidine-3-carboxamide (500 mg, 987.0 umol) and 2-chloro-6-(2,6-dioxopiperidin-3-yl)-5H-pyrrolo[3,4-b]pyridine-5,7(6H)-dione (300 mg, 1.0 mmol) in dimethyl sulfoxide (5 mL) was added diisopropylethylamine (1.86 g, 14.4 mmol, 2.5 mL) and the mixture was stirred at 100° C. for 12 h. The reaction mixture was quenched by water (20 mL) and extracted with dichloromethane (5×50 mL), The organic phase was washed with brine (50 mL), dried over anhydrous sodium sulfate, and concentrated under reduced pressure. The residue was purified by prep-HPLC (Phenomenex luna C18 150*25 mm*10 um; [water (0.2% formic acid)-formic acid]; B %: 20%-40%, 10 min) to afford N-(2-(1-((1-(6-(2,6-dioxopiperidin-3-yl)-5,7-dioxo-6,7-dihydro-5H-pyrrolo[3,4-b]pyridin-2-yl)-3-fluoroazetidin-3-yl)methyl)piperidin-4-yl)-6-isopropoxy-2H-indazol-5-yl)pyrazolo[1,5-a]pyrimidine-3-carboxamide (274.4 mg, 35%) as a yellow solid. MS (ESI) m/z: 764.3 [M+H]⁺; ¹H NMR (400 MHz, DMSO-d₆) δ 11.10 (br s, 1H), 10.59 (s, 1H), 9.38 (d, J=6.8 Hz, 1H), 8.87 (br d, J=3.6 Hz, 1H), 8.72 (d, J=14.8 Hz, 2H), 8.30 (s, 1H), 7.97 (d, J=8.8 Hz, 1H), 7.33 (dd, J=4.0, 6.8 Hz, 1H), 7.13 (s, 1H), 6.73 (d, J=8.4 Hz, 1H), 5.11 (dd, J=5.2, 12.8 Hz, 1H), 4.90-4.79 (m, 1H), 4.44-4.21 (m, 5H), 3.06 (br d, J=11.6 Hz, 2H), 3.00 (br s, 1H), 2.94 (br s, 1H), 2.91-2.82 (m, 1H), 2.64-2.53 (m, 4H), 2.42 (br t, J=10.8 Hz, 2H), 2.20-1.95 (m, 5H), 1.47 (d, J=6.0 Hz, 6H).

### Preparation of Example 412: N-(2-((2S)-2-(1-((1-(2-(2,6-dioxopiperidin-3-yl)-1,3-dioxoisoindolin-5-yl)azetidin-3-yl)methyl)piperidin-4-yl)-2-fluoroethyl)-6-morpholino-1-oxoisoindolin-5-yl)pyrazolo[1,5-a]pyrimidine-3-carboxamide

Step 1: (S)-2-amino-N-methyl-3-phenylpropanamide

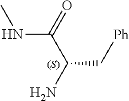

A mixture of (S)-methyl 2-amino-3-phenylpropanoate hydrochloride (5 g, 23.2 mmol) and methylamine (8.820 g, 93.7 mmol, 33% in ethyl alcohol) was stirred at 25° C. for 12 h. The mixture was concentrated to afford (S)-2-amino-N-methyl-3-phenylpropanamide (4 g, crude) as a yellow oil, which was used in the next step without further purification. MS (ESI) m/z: 179.2 [M+H]⁺

Step 2: (S)-5-benzyl-2,2,3-trimethylimidazolidin-4-one

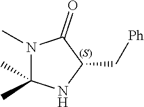

To a solution of (S)-2-amino-N-methyl-3-phenylpropanamide (4 g, 22.4 mmol, crude) in methyl alcohol (30 mL) and acetone (10 mL) was added p-toluenesulfonic acid (200 mg, 1.2 mmol) and stirred at 80° C. for 12 h. The solvent was removed under reduced pressure and the residue was taken up in dichloromethane (100 mL) and saturated sodium carbonate solution (100 mL), separated the layers. The aqueous layer was extracted with dichloromethane (2×50 mL). The combined organic phase was washed with brine (50 mL), dried over anhydrous sodium sulfate, filtered, and concentrated under vacuum. The residue was purified by semi-preparative reverse phase HPLC (column: Waters Xbridge BEH C18 250*50 mm*10 um; mobile phase: [water (0.05% ammonia hydroxide v/v)-acetonitrile]; B %: 15%-45%, 17 min) to afford (S)-5-benzyl-2,2,3-trimethylimidazolidin-4-one (3 g, 60%) as a brown oil. MS (ESI) m/z: 219.2 [M+H]⁺; ¹H NMR (400 MHz, CDCl₃) δ 7.23-7.34 (m, 5H) 3.79-3.85 (m, 1H) 3.14-3.21 (m, 1H) 3.00-3.07 (m, 1H) 2.78 (s, 3H) 1.29 (s, 3H) 1.18 (s, 3H).

Step 3: (S)-tert-butyl 4-(1-fluoro-2-hydroxyethyl)piperidine-1-carboxylate

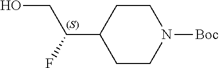

To a solution of (S)-5-benzyl-2,2,3-trimethylimidazolidin-4-one (1.54 g, 7.0 mmol) in tetrahydrofuran (200 mL) and isopropyl alcohol (25 mL) was added 2,2-dichloroacetic acid (907.65 mg, 7.0 mmol, 578.12 uL) and N-fluorobenzenesulfonimide (55.49 g, 176.0 mmol). The mixture was stirred at 25° C. until homogeneous then cooled to −10° C. Tert-butyl 4-(2-oxoethyl)piperidine-1-carboxylate (8 g, 35.2 mmol) was added at −10° C. and the reaction mixture stirred at −10° C. for 12 h. The reaction was cooled to −78° C., diluted with petroleum ether (350 mL) and filtered through a pad of silica gel, eluting with petroleum ether (100 mL). Dimethyl sulfide (25.54 g, 411.1 mmol, 30.19 mL) was added forming a white precipitate. The resulting mixture was washed with saturation sodium bicarbonate (3×150 mL) and brine (150 mL) and dried over anhydrous sodium sulfate, filtered, and concentrated in vacuo. The resulting oil was dissolved in dichloromethane (150 mL) and ethyl alcohol (100 mL). Sodium borohydride (3.29 g, 87.0 mmol) was added and stirred at 25° C. for 1 h. The reaction was cooled to 0° C. and saturation ammonium chloride (250 mL) was added. The mixture was warmed to 25° C. and stirred vigorously for 1 h. Then extracted with dichloromethane (3×100 mL) and the combined organics were further washed with saturation sodium bicarbonate (3×150 mL) and brine (150 mL), dried over anhydrous sodium sulfate, filtered, and concentrated. The residue was purified by silica gel column chromatography (petroleum ether/ethyl acetate=5/1 to 2/1) to afford (S)-tert-butyl 4-(1-fluoro-2-hydroxyethyl)piperidine-1-carboxylate (7.5 g, 86%) as a light yellow oil. MS (ESI) m/z: 192.2 [M−55]; ¹H NMR (400 MHz, CDCl₃) δ 4.22-4.40 (m, 1H) 4.12-4.20 (m, 2H) 3.70-3.87 (m, 2H) 2.63-2.76 (m, 2H) 1.81-1.87 (m, 2H) 1.55-1.62 (m, 1H) 1.46 (s, 9H) 1.25-1.33 (m, 2H).

Step 4: (S)-tert-butyl 4-(1-fluoro-2-((methylsulfonyl)oxy)ethyl)piperidine-1-carboxylate

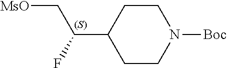

To a solution of (S)-tert-butyl 4-(1-fluoro-2-hydroxyethyl)piperidine-1-carboxylate (7.0 g, 28.3 mmol) and N,N-diisopropylethylamine (14.63 g, 113.2 mmol, 19.72 mL) in dichloromethane (80 mL) was added methanesulfonyl chloride (11.24 g, 98.1 mmol, 7.59 mL) at 0° C. and stirred at 25° C. for 12 h. The mixture was concentrated. The residue was purified by silica gel column chromatography (petroleum ether/ethyl acetate=5/1 to 2/1) to afford (S)-tert-butyl 4-(1-fluoro-2-hydroxyethyl)piperidine-1-carboxylate (8.5 g, 92%) as a yellow oil. MS (ESI) m/z: 270.0 [M−55]⁺; ¹H NMR (400 MHz, CDCl₃) δ 4.42-4.56 (m, 1H) 4.32-4.41 (m, 2H) 4.14-4.21 (m, 2H) 3.07 (s, 3H) 2.64-2.75 (m, 2H) 1.80-1.93 (m, 2H) 1.55-1.64 (m, 1H) 1.45 (s, 9H) 1.27-1.37 (m, 2H).

Step 5: (S)-tert-butyl 4-(2-azido-1-fluoroethyl)piperidine-1-carboxylate

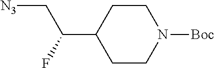 [SMILES: CC(C)(C)OC(=O)N1CCC([C@H](F)CN=[N+]=[N-])CC1]

A mixture of (S)-tert-butyl 4-(1-fluoro-2-hydroxyethyl)piperidine-1-carboxylate (8.5 g, 26.1 mmol) and sodium azide (8.98 g, 138.1 mmol) in dimethyl sulfoxide (50 mL) was stirred at 80° C. for 12 h. The mixture was poured into ice-water (500 mL). The aqueous phase was extracted with ethyl acetate (3×100 mL). The combined organic phase was washed with brine (3×100 mL), dried over anhydrous sodium sulfate, filtered, and concentrated under vacuum. The residue was purified by silica gel column chromatography (petroleum ether/ethyl acetate=10/1 to 5/1) to afford (S)-tert-butyl 4-(2-azido-1-fluoroethyl)piperidine-1-carboxylate (7 g, 98%) as a light yellow oil. MS (ESI) m/z: 217.0 [M−55]⁺.

Step 6: (S)-tert-butyl 4-(2-amino-1-fluoroethyl)piperidine-1-carboxylate

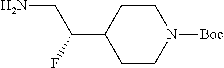 [SMILES: CC(C)(C)OC(=O)N1CCC([C@H](F)CN)CC1]

To a solution of (S)-tert-butyl 4-(2-azido-1-fluoroethyl)piperidine-1-carboxylate (7 g, 25.7 mmol) in trifluoroethanol (70 mL) was added 10% palladium on carbon (2.0 g) under nitrogen atmosphere. The suspension was degassed and purged with hydrogen for 3 times. The mixture was stirred under hydrogen (15 Psi) at 25° C. for 12 h. The reaction mixture was filtered and washed with ethanol (3×20 mL). The filtrate was concentrated to afford (S)-tert-butyl 4-(2-amino-1-fluoroethyl)piperidine-1-carboxylate (5 g, 78%) as a light yellow oil, which was used in the next step without further purification.

Step 7: (S)-tert-butyl 4-(2-(6-chloro-5-nitro-1-oxoisoindolin-2-yl)-1-fluoroethyl)piperidine-1-carboxylate

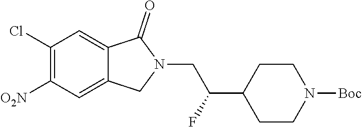 [SMILES: CC(C)(C)OC(=O)N1CCC([C@H](F)CN2Cc3cc([N+](=O)[O-])c(Cl)cc3C2=O)CC1]

To a solution of (S)-tert-butyl 4-(2-amino-1-fluoroethyl)piperidine-1-carboxylate (958.10 mg, 3.9 mmol) and N,N-diisopropylethylamine (1.19 g, 9.2 mmol, 1.60 mL) in N,N-dimethylformamide (20 mL) was dropwise added a solution of methyl 2-(bromomethyl)-5-chloro-4-nitrobenzoate (0.8 g, 2.6 mmol) in N,N-dimethylformamide (8 mL) at −10° C. The reaction mixture was stirred at 0° C. for 1 h and heated to 70° C. for 11 h. The mixture was concentrated. The residue was purified by silica gel column chromatography (petroleum ether/ethyl acetate=3/1 to 1/1) to afford (S)-tert-butyl 4-(2-(6-chloro-5-nitro-1-oxoisoindolin-2-yl)-1-fluoroethyl)piperidine-1-carboxylate (1.1 g, 96%) as a light yellow solid. MS (ESI) m/z: 342.2 [M−100+1]⁺; ¹H NMR (400 MHz, CDCl₃) δ 8.02 (s, 1H) 7.91 (s, 1H) 4.42-4.69 (m, 3H) 4.18 (br d, J=13.2 Hz, 2H) 3.93-4.07 (m, 1H) 3.70-3.84 (m, 1H) 2.62-2.75 (m, 2H) 1.73-1.89 (m, 3H) 1.46 (s, 9H) 1.30-1.39 (m, 2H).

Step 8: (S)-tert-butyl 4-(1-fluoro-2-(6-morpholino-5-nitro-1-oxoisoindolin-2-yl)ethyl)piperidine-1-carboxylate

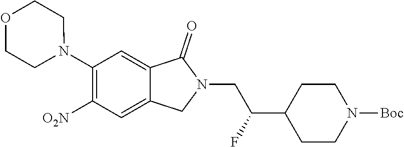 [SMILES: CC(C)(C)OC(=O)N1CCC([C@H](F)CN2Cc3cc([N+](=O)[O-])c(N4CCOCC4)cc3C2=O)CC1]

To a solution of morpholine (1.10 g, 12.6 mmol, 1.11 mL) and (S)-tert-butyl 4-(2-(6-chloro-5-nitro-1-oxoisoindolin-2-yl)-1-fluoroethyl)piperidine-1-carboxylate (1.1 g, 2.5 mmol) in dimethyl sulfoxide (2 mL) was dropwise added N,N-diisopropylethylamine (1.29 g, 10.0 mmol, 1.73 mL) and heated to 100° C. for 12 h. The mixture was poured into ice-water (30 mL). The aqueous phase was extracted with dichloromethane (3×30 mL). The combined organic phase was washed with brine (3×40 mL), dried over anhydrous sodium sulfate, filtered, and concentrated under vacuum. The residue was purified by silica gel column chromatography (petroleum ether/ethyl acetate=2/1 to 1/1) to afford (S)-tert-butyl 4-(1-fluoro-2-(6-morpholino-5-nitro-1-oxoisoindolin-2-yl)ethyl)piperidine-1-carboxylate (1.2 g, 97%) as a yellow solid. MS (ESI) m/z: 437.1 [M−55]⁺; ¹H NMR (400 MHz, CDCl₃) δ 7.79 (s, 1H) 7.64 (s, 1H) 4.41-4.62 (m, 3H) 4.08-4.27 (m, 2H) 3.90-4.04 (m, 1H) 3.81-3.89 (m, 4H) 2.99-3.14 (m, 4H) 2.68 (br d, J=11.2 Hz, 2H) 1.71-1.90 (m, 3H) 1.46 (s, 9H) 1.34 (qd, J=12.8, 4.4 Hz, 2H).

Step 9: (S)-tert-butyl 4-(2-(5-amino-6-morpholino-1-oxoisoindolin-2-yl)-1-fluoroethyl)piperidine-1-carboxylate

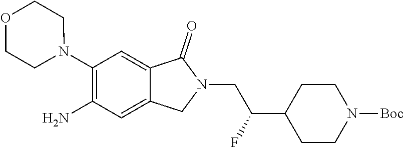 [SMILES: CC(C)(C)OC(=O)N1CCC([C@H](F)CN2Cc3cc(N)c(N4CCOCC4)cc3C2=O)CC1]

To a solution of (S)-tert-butyl 4-(1-fluoro-2-(6-morpholino-5-nitro-1-oxoisoindolin-2-yl)ethyl)piperidine-1-carboxylate (1.1 g, 2.2 mmol) in trifluoroethanol (20 mL) was added 10% palladium on carbon (0.5 g) under nitrogen atmosphere. The suspension was degassed and purged with hydrogen for 3 times. The mixture was stirred under hydrogen (15 Psi) at 25° C. for 2 h. The reaction mixture was filtered and washed with ethanol (3×30 mL). The filtrate was concentrated to afford (S)-tert-butyl 4-(2-(5-amino-6-morpholino-1-oxoisoindolin-2-yl)-1-fluoroethyl)piperidine-1-carboxylate (1 g, 96%) as a white solid. MS (ESI) m/z: 407.1 [M+H]⁺.

Step 10: (S)-tert-butyl 4-(1-fluoro-2-(6-morpholino-1-oxo-5-(pyrazolo[1,5-a]pyrimidine-3-carboxamido)isoindolin-2-yl)ethyl)piperidine-1-carboxylate

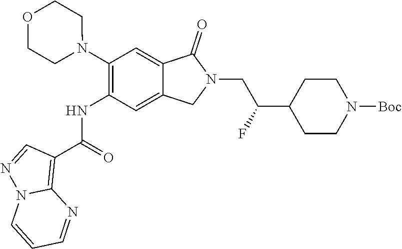 [SMILES: CC(C)(C)OC(=O)N1CCC([C@H](F)CN2Cc3cc(NC(=O)c4cnn5cccnc45)c(N4CCOCC4)cc3C2=O)CC1]

To a solution of pyrazolo[1,5-a]pyrimidine-3-carboxylic acid (0.5 g, 3.1 mmol) in pyridine (10 mL) was added (S)-tert-butyl 4-(2-(5-amino-6-morpholino-1-oxoisoindolin-2-yl)-1-fluoroethyl)piperidine-1-carboxylate (0.95 g, 2.1 mmol) and 1-(3-dimethylaminopropyl)-3-ethylcarbodiimide hydrochloride (2 g, 10.43 mmol). The reaction mixture was stirred at 70° C. for 12 h, then concentrated. The residue was purified by silica gel column chromatography (petroleum ether/ethyl acetate=2/1 to dichloromethane/methyl alcohol=20/1) to afford (S)-tert-butyl 4-(1-fluoro-2-(6-morpholino-1-oxo-5-(pyrazolo[1,5-a]pyrimidine-3-carboxamido)isoindolin-2-yl)ethyl)piperidine-1-carboxylate (1.1 g, 81%) as a light yellow solid. MS (ESI) m/z: 608.3 [M+H]⁺.

Step 11: (S)—N-(2-(2-fluoro-2-(piperidin-4-yl)ethyl)-6-morpholino-1-oxoisoindolin-5-yl)pyrazolo[1,5-a]pyrimidine-3-carboxamide

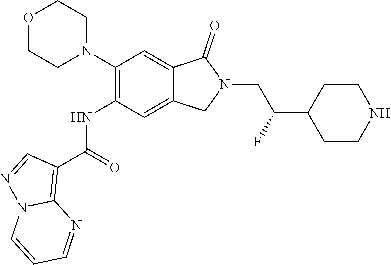 [SMILES: O=C(Nc1cc2c(cc1N1CCOCC1)C(=O)N(C[C@@H](F)C1CCNCC1)C2)c1cnn2cccnc12]

To a solution of (S)-tert-butyl 4-(1-fluoro-2-(6-morpholino-1-oxo-5-(pyrazolo[1,5-a]pyrimidine-3-carboxamido)isoindolin-2-yl)ethyl)piperidine-1-carboxylate (1.1 g, 1.8 mmol) in dichloromethane (10 mL) was added hydrochloric/methyl alcohol (4 M, 10 mL). The reaction mixture was stirred at 25° C. for 1 h. The residue was diluted with dichloromethane/methyl alcohol (10/1, 30 mL), adjusted the pH to 8 with N,N-diisopropylethylamine, dried over anhydrous sodium sulfate, filtered and concentrated. The residue was purified by silica gel column chromatography (dichloromethane/methyl alcohol=10/1 to 5/1 (with 0.5% N,N-diisopropylethylamine)) to afford (S)—N-(2-(2-fluoro-2-(piperidin-4-yl)ethyl)-6-morpholino-1-oxoisoindolin-5-yl)pyrazolo[1,5-a]pyrimidine-3-carboxamide (0.9 g, 97%) as a light yellow solid. MS (ESI) m/z: 508.2 [M+H]⁺.

Step 12: ((S)-tert-butyl 3-((4-(1-fluoro-2-(6-morpholino-1-oxo-5-(pyrazolo[1,5-a]pyrimidine-3-carboxamido)isoindolin-2-yl)ethyl)piperidin-1-yl)methyl)azetidine-1-carboxylate

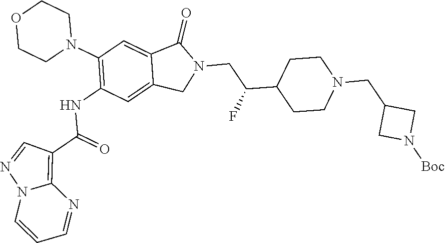 [SMILES: CC(C)(C)OC(=O)N1CC(CN2CCC([C@H](F)CN3Cc4cc(NC(=O)c5cnn6cccnc56)c(N5CCOCC5)cc4C3=O)CC2)C1]

To a solution of (S)—N-(2-(2-fluoro-2-(piperidin-4-yl)ethyl)-6-morpholino-1-oxoisoindolin-5-yl)pyrazolo[1,5-a]pyrimidine-3-carboxamide (0.15 g, 295.5 umol) and tert-butyl 3-formylazetidine-1-carboxylate (0.17 g, 917.8 umol) in 1,2-dichloroethane (3 mL) and dimethyl sulfoxide (3 mL) was added acetic acid (35.49 mg, 591.1 umol, 33.80 uL). After stirred at 25° C. for 0.5 h, sodium triacetoxyborohydride (250.54 mg, 1.2 mmol) was added to the above reaction mixture. The resulting mixture was stirred at 25° C. for 1.5 h. The mixture was poured into ice-water (20 mL) and adjusted the pH to 8 with saturated sodium bicarbonate at 0° C. The aqueous phase was extracted with dichloromethane (3×20 mL). The combined organic phase was washed with brine (3×20 mL), dried over anhydrous sodium sulfate, filtered and concentrated under vacuum. The residue was purified by prep-Thin-layer chromatography (dichloromethane/methyl alcohol=8/1) to afford (S)-tert-butyl 3-((4-(1-fluoro-2-(6-morpholino-1-oxo-5-(pyrazolo[1,5-a]pyrimidine-3-carboxamido)isoindolin-2-yl)ethyl)piperidin-1-yl)methyl)azetidine-1-carboxylate (0.18 g, 90%) as a white solid. MS (ESI) m/z: 677.4 [M+H]⁺.

Step 13: (S)—N-(2-(2-(1-(azetidin-3-ylmethyl)piperidin-4-yl)-2-fluoroethyl)-6-morpholino-1-oxoisoindolin-5-yl)pyrazolo[1,5-a]pyrimidine-3-carboxamide

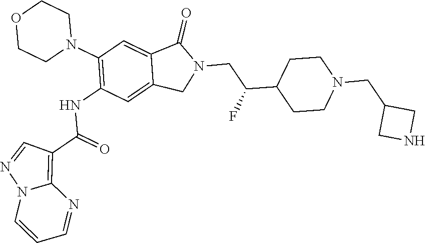 [SMILES: O=C(Nc1cc2c(cc1N1CCOCC1)C(=O)N(C[C@@H](F)C1CCN(CC3CNC3)CC1)C2)c1cnn2cccnc12]

To a solution of (S)-tert-butyl 3-((4-(1-fluoro-2-(6-morpholino-1-oxo-5-(pyrazolo[1,5-a]pyrimidine-3-carboxamido)isoindolin-2-yl)ethyl)piperidin-1-yl)methyl)azetidine-1-carboxylate (0.18 g, 266.0 umol) in dichloromethane (5 mL) was added trifluoroacetic acid (7.70 g, 67.5 mmol, 5 mL) and was stirred at 25° C. for 1 h. The mixture was concentrated to afford (S)—N-(2-(2-(1-(azetidin-3-ylmethyl)piperidin-4-yl)-2-fluoroethyl)-6-morpholino-1-oxoisoindolin-5-yl)pyrazolo[1,5-a]pyrimidine-3-carboxamide trifluoroacetate (0.18 g, 97%) as a light yellow gum, which was used in the next step without further purification. MS (ESI) m/z: 577.3 [M+H]⁺.

Step 14: N-(2-((2S)-2-(1-((1-(2-(2,6-dioxopiperidin-3-yl)-1,3-dioxoisoindolin-5-yl)azetidin-3-yl)methyl)piperidin-4-yl)-2-fluoroethyl)-6-morpholino-1-oxoisoindolin-5-yl)pyrazolo[1,5-a]pyrimidine-3-carboxamide

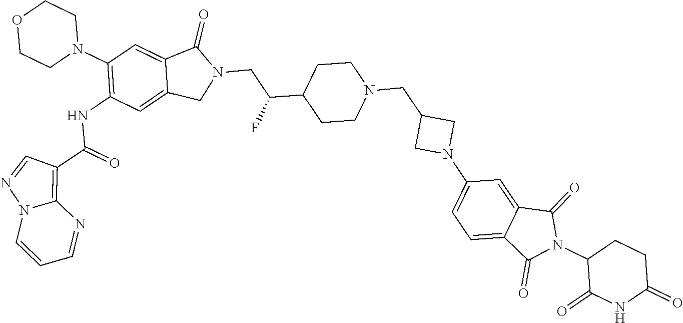 [SMILES: O=C1CCC(N2C(=O)c3ccc(N4CC(CN5CCC([C@H](F)CN6Cc7cc(NC(=O)c8cnn9cccnc89)c(N8CCOCC8)cc7C6=O)CC5)C4)cc3C2=O)C(=O)N1]

To a solution of (S)—N-(2-(2-(1-(azetidin-3-ylmethyl)piperidin-4-yl)-2-fluoroethyl)-6-morpholino-1-oxoisoindolin-5-yl)pyrazolo[1,5-a]pyrimidine-3-carboxamide trifluoroacetate (0.18 g, 260.6 umol) in dimethyl sulfoxide (5 mL) was added N,N-diisopropylethylamine (742.00 mg, 5.7 mmol, 1 mL) and 2-(2,6-dioxo-3-piperidyl)-5-fluoro-isoindoline-1,3-dione (90 mg, 325.8 umol). The reaction mixture was stirred at 80° C. for 12 h, then poured into ice-water (20 mL). The aqueous phase was extracted with dichloromethane (3×20 mL). The combined organic phase was washed with brine (2×20 mL), dried over anhydrous sodium sulfate, filtered, and concentrated under vacuum. The residue was purified by prep-Thin-layer chromatography (dichloromethane/methyl alcohol=8/1) to afford N-(2-((2S)-2-(1-((1-(2-(2,6-dioxopiperidin-3-yl)-1,3-dioxoisoindolin-5-yl)azetidin-3-yl)methyl)piperidin-4-yl)-2-fluoroethyl)-6-morpholino-1-oxoisoindolin-5-yl)pyrazolo[1,5-a]pyrimidine-3-carboxamide (89.3 mg, 39%) as a yellow solid. MS (ESI) m/z: 833.3 [M+H]⁺; ¹H NMR (400 MHz, DMSO-d₆) δ 11.10 (s, 1H) 10.90 (s, 1H) 9.41 (dd, J=6.8, 1.6 Hz, 1H) 8.99 (dd, J=4.4, 1.6 Hz, 1H) 8.75 (s, 2H) 7.58-7.64 (m, 2H) 7.39 (dd, J=7.2, 4.4 Hz, 1H) 6.75 (s, 1H) 6.62 (dd, J=8.4, 1.6 Hz, 1H) 5.77 (s, 1H) 5.06 (dd, J=12.8, 5.2 Hz, 1H) 4.50-4.65 (m, 3H) 4.11 (br t, J=8.0 Hz, 2H) 3.90 (br s, 4H) 3.73-3.87 (m, 2H) 3.63-3.68 (m, 2H) 2.85-3.01 (m, 9H) 2.55-2.61 (m, 3H) 1.98-2.04 (m, 1H) 1.89 (br t, J=10.4 Hz, 2H) 1.70-1.82 (m, 2H) 1.55 (br s, 1H) 1.29-1.38 (m, 2H).

TABLE 14
The following compounds may be prepared in an analogous fashion as described for the
synthesis of Example 412.

| | | ¹H NMR |
|---|---|---|
| | | All spectra acquired at 400 |
| Example | MS | MHz unless otherwise indicated |
| Example 412 | See Above | |
| 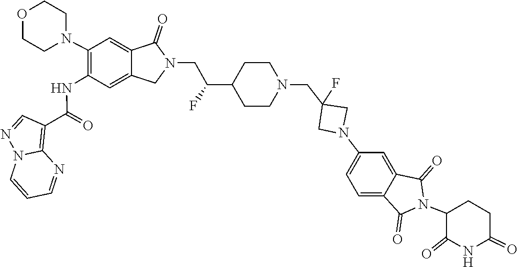 [SMILES: O=C1CCC(N2C(=O)c3ccc(N4CC(F)(CN5CCC([C@H](F)CN6Cc7cc(NC(=O)c8cnn9cccnc89)c(N8CCOCC8)cc7C6=O)CC5)C4)cc3C2=O)C(=O)N1] | 851.31 | DMSO-d₆ δ = 11.10 (s, 1 H) 10.91 (s, 1 H) 9.42 (d, J = 7.2 Hz, 1 H) 9.00 (d, J = 4.0 Hz, 1 H) 8.76 (d, J = 2.0 Hz, 2 H) 7.67 (d, J = 8.4 Hz, 1 H) 7.60 (s, 1 H) 7.39 (dd, J = 6.8, 4.4 Hz, 1 H) 6.90 (s, 1 H) 6.75 (br d, J = 8.4 Hz, 1 H) 5.76 (s, 1 H) 5.07 (dd, J = 12.8, 5.6 Hz, 1 H) 4.50-4.64 (m, 3 H) 4.09- 4.22 (m, 4 H) 3.90 (br s, 4 H) 3.72- 3.82 (m, 2 H) 2.83-2.95 (m, 9 H) 2.53-2.61 (m, 3 H) 2.11 (br t, J = 10.4 Hz, 2 H) 2.01 (br d, J = 6.4 Hz, 1 H) 1.68-1.79 (m, 2H) 1.33-1.39 (m, 1 H) 1.23 (br s, 1 H) |
| Example 413 | | |
| 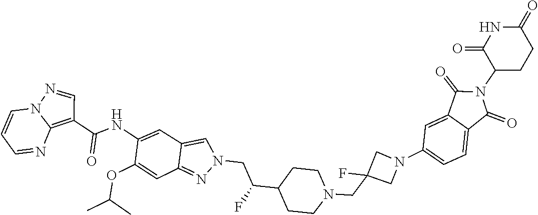 [SMILES: CC(C)Oc1cc2nn(C[C@@H](F)C3CCN(CC4(F)CN(c5ccc6c(c5)C(=O)N(C5CCC(=O)NC5=O)C6=O)C4)CC3)cc2cc1NC(=O)c1cnn2cccnc12] | 809.12 | DMSO-d₆ δ = 10.97 (s, 1H), 10.56 (s, 1H), 9.35 (dd, J = 1.2, 7.2 Hz, 1H), 9.29-9.14 (m, 1H), 8.87 (d, J = 2.8 Hz, 1H), 8.76 (s, 1H), 8.69 (s, 1H), 8.26 (s, 1H), 7.67 (d, J = 8.4 Hz, 1H), 7.34 (dd, J = 4.0, 7.2 Hz, 1H), 7.11 (s, 1H), 6.81 (s, 1H), 6.68 (br d, J = 8.8 Hz, 1H), 5.04 (dd, J = 5.2, 12.8 Hz, 1H), 4.89-4.83 (m, 1H), 4.70 (br d, J = 6.8 Hz, 1H), 4.64 (br d, J = 5.4 Hz, 1H), 4.25 (br t, J = 8.0 Hz, 2H), 3.84 (br t, J = 6.4 Hz, 2H), 3.52 (br d, J = 9.2 Hz, 2H), 3.45 (br d, J = 2.4 Hz, 2H), 3.07-2.91 (m, 3H), 2.90-2.81 (m, 1H), 2.62 (br s, |
| Example 414 | | 1H), 2.58 (brs, 1H), 2.07-1.99 (m, 3H), |
| | | 1.97-1.81 (m, 2H), 1.73-1.62 (m, 2H), |
| | | 1.49 (dd, J = 2.0, 6.0 Hz, 6H) |
| 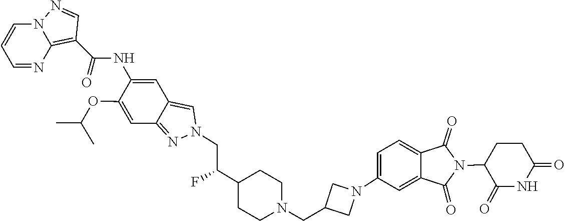 [SMILES: CC(C)Oc1cc2nn(C[C@@H](F)C3CCN(CC4CN(c5ccc6c(c5)C(=O)N(C5CCC(=O)NC5=O)C6=O)C4)CC3)cc2cc1NC(=O)c1cnn2cccnc12] | 791.12 | DMSO-d₆ δ = 10.97 (s, 1H), 10.56 (s, 1H), 9.35 (dd, J = 1.2, 7.2 Hz, 1H), 9.29-9.14 (m, 1H), 8.87 (d, J = 2.8 Hz, 1H), 8.76 (s, 1H), 8.69 (s, 1H), 8.26 (s, 1H), 7.67 (d, J = 8.4 Hz, 1H), 7.34 (dd, J = 4.0, 7.2 Hz, 1H), 7.11 (s, 1H), 6.81 (s, 1H), 6.68 (br d, J = 8.8 Hz, 1H), 5.04 (dd, J = 5.2, 12.8 Hz, 1H), 4.89-4.83 (m, 1H), 4.70 (br d, J = 6.8 Hz, 1H), 4.64 (br d, J = 5.4 Hz, 1H), 4.25 (br t, J = 8.0 Hz, 2H), 3.84 (br t, J = 6.4 Hz, 2H), 3.52 (br d, J = 9.2 Hz, 2H), 3.45 (br d, J = 2.4 Hz, 2H), 3.07-2.91 (m, 3H), 2.90-2.81 (m, 1H), 2.62 (br s, |
| Example 415 | | 1H), 2.58 (brs, 1H), 2.07-1.99 (m, 3H), |
| | | 1.97-1.81 (m,2H), 1.73-1.62 (m, |
| | | 2H), 1.49 (dd, J = 2.0, 6.0 Hz, 6H) |

### Preparation of Example 416: N-[2-[1-[[1-[2-(2,6-dioxo-3-piperidyl)-1-oxo-isoindolin-5-yl]-3-fluoro-azetidin-3-yl]methyl]-4-piperidyl]-6-isopropoxy-pyrazolo[3,4-b]pyridin-5-yl]pyrazolo[1,5-a]pyrimidine-3-carboxamide

Step 1: 6-chloro-2-fluoro-pyridine-3-carbaldehyde

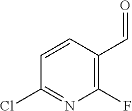

To a solution of 2-chloro-6-fluoro-pyridine (10 g, 76.0 mmol) in tetrahydrofuran (100 mL) was added lithium diisopropylamide (2 M, 57 mL) at −78° C., 30 min later N,N-dimethylformamide (11.11 g, 152.1 mmol, 11.70 mL) was added to the mixture. The reaction mixture was stirred at −78° C. for 1 h. 4 M hydrogen chloride in ethyl acetate (80 mL) was added to the mixture to adjust the PH to 1, then warmed to 25° C., water (50 mL) was added. The reaction mixture was stirred at 25° C. for 0.5 h. The reaction mixture was extracted with ethyl acetate (3×50 mL). The combined organic layers were washed with brine (3×50 mL), dried over anhydrous sodium sulfate, filtered, and concentrated under reduced pressure. The residue was purified by flash silica gel chromatography (0-30% ethyl acetate/petroleum ether gradient @65 mL/min) to afford 6-chloro-2-fluoro-pyridine-3-carbaldehyde (6.3 g, 51%) as light-yellow solid. ¹H NMR (400 MHz, CDCl₃) δ 10.27 (s, 1H), 8.27 (dd, J=8.0, 8.8 Hz, 1H), 7.41 (d, J=8.0 Hz, 1H).

Step 2: 6-chloro-1H-pyrazolo[3,4-b]pyridine

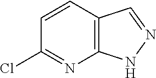

To a solution of 6-chloro-2-fluoro-pyridine-3-carbaldehyde (6.2 g, 38.9 mmol) in tetrahydrofuran (30 mL) was added hydrazine hydrate (4.93 g, 96.5 mmol, 4.79 mL). The reaction mixture was stirred at 60° C. for 12 h, then concentrated under reduced pressure. The residue was poured into ice water, the mixture was filtered and dried in vacuum to afford 6-chloro-1H-pyrazolo[3,4-b]pyridine (5.5 g, 92%) as yellow solid. ¹H NMR (400 MHz, DMSO-d₆) δ 13.84 (s, 1H), 8.29 (d, J=8.4 Hz, 1H), 8.20 (s, 1H), 7.25 (d, J=8.3 Hz, 1H).

Step 3: tert-butyl 4-(6-chloropyrazolo[3,4-b]pyridin-2-yl)piperidine-1-carboxylate

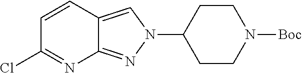 [SMILES: CC(C)(C)OC(=O)N1CCC(n2cc3ccc(Cl)nc3n2)CC1]

To a solution of 6-chloro-1H-pyrazolo[3,4-b]pyridine (5.5 g, 35.8 mmol) and tert-butyl 4-iodopiperidine-1-carboxylate (22.29 g, 71.6 mmol) in N,N-dimethylformamide (60 mL) was added cesium carbonate (35.01 g, 107.4 mmol). The reaction mixture was stirred at 80° C. for 12 h. The reaction was quenched by water (10 mL), then diluted with water (20 mL) and extracted with ethyl acetate (3×30 mL). The combined organic layers were washed with brine (3×40 mL), dried over anhydrous sodium sulfate, filtered, and concentrated under reduced pressure. The residue was purified by flash silica gel chromatography (0˜70% ethyl acetate/petroleum ether gradient @60 mL/min) to afford tert-butyl 4-(6-chloropyrazolo[3,4-b]pyridin-2-yl)piperidine-1-carboxylate (1.2 g, 9%) as light yellow solid. MS (ESI) m/z: 281.1 [M−56]⁺; ¹H NMR (400 MHz, CDCl₃) δ 8.01-7.92 (m, 2H), 7.06 (d, J=8.8 Hz, 1H), 4.69-4.46 (m, 1H), 4.31 (d, J=13.2 Hz, 2H), 3.08-2.89 (m, 2H), 2.33-2.20 (m, 2H), 2.19-2.02 (m, 2H), 1.49 (s, 9H).

Step 4: tert-butyl 4-(6-isopropoxypyrazolo[3,4-b]pyridin-2-yl)piperidine-1-carboxylate

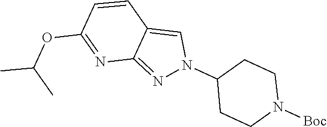 [SMILES: CC(C)Oc1ccc2cn(C3CCN(C(=O)OC(C)(C)C)CC3)nc2n1]

To a solution of tert-butyl 4-(6-chloropyrazolo[3,4-b]pyridin-2-yl)piperidine-1-carboxylate (700 mg, 2.1 mmol) and propan-2-ol (250 mg, 4.2 mmol) in dioxane (10 mL) was added RuPhos Pd G4 (40 mg) and sodium tert-butoxide (2 M, 3 mL). The reaction mixture was stirred at 90° C. for 12 h under nitrogen, then concentrated under reduced pressure. The residue was purified by flash silica gel chromatography (0˜50% ethyl acetate/petroleum ether gradient @30 mL/min) to afford tert-butyl 4-(6-isopropoxypyrazolo[3,4-b]pyridin-2-yl)piperidine-1-carboxylate (400 mg, 53%) as light yellow solid. MS (ESI) m/z: 361.2 [M+H]⁺.

Step 5: tert-butyl 4-(5-bromo-6-isopropoxy-pyrazolo[3,4-b]pyridin-2-yl)piperidine-1-carboxylate

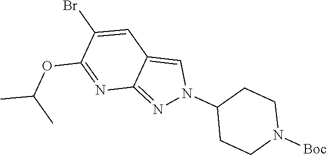 [SMILES: CC(C)Oc1nc2nn(C3CCN(C(=O)OC(C)(C)C)CC3)cc2cc1Br]

To a solution of tert-butyl 4-(6-isopropoxypyrazolo[3,4-b]pyridin-2-yl)piperidine-1-carboxylate (50 mg, 0.1 mmol) in acetonitrile (1 mL) was added 1-bromopyrrolidine-2,5-dione (25 mg, 0.1 mmol). The reaction mixture was stirred at 25° C. for 12 h, then concentrated under reduced pressure. The residue was purified by prep-Thin layer chromatography (petroleum ether:ethyl acetate=2:1) to afford tert-butyl 4-(5-bromo-6-isopropoxy-pyrazolo[3,4-b]pyridin-2-yl)piperidine-1-carboxylate (20 mg, 32%) as light-yellow solid. MS (ESI) m/z: 439.1 [M+H]⁺; ¹H NMR (400 MHz, CDCl₃) δ 8.10 (s, 1H), 7.75 (s, 1H), 5.68-5.42 (m, 1H), 4.52-4.37 (m, 1H), 4.29 (s, 2H), 3.04-2.84 (m, 2H), 2.22-2.06 (m, 4H), 1.48 (s, 9H), 1.43 (d, J=6.4 Hz, 6H).

Step 6: tert-butyl 4-[6-isopropoxy-5-(pyrazolo[1,5-a]pyrimidine-3-carbonylamino)pyrazolo[3,4-b]pyridin-2-yl]piperidine-1-carboxylate

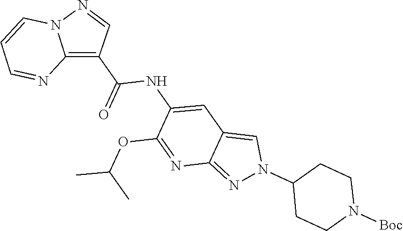 [SMILES: CC(C)Oc1nc2nn(C3CCN(C(=O)OC(C)(C)C)CC3)cc2cc1NC(=O)c1cnn2cccnc12]

To a solution of pyrazolo[1,5-a]pyrimidine-3-carboxamide (140 mg, 0.9 mmol) and tert-butyl 4-(5-bromo-6-isopropoxy-pyrazolo[3,4-b]pyridin-2-yl)piperidine-1-carboxylate (380 mg, 0.9 mmol) in dioxane (10 mL) was added BrettPhos Pd G4 (80 mg, 0.09 mmol) and cesium carbonate (564 mg, 1.7 mmol). The reaction mixture was stirred at 90° C. for 12 h under nitrogen, then concentrated under reduced pressure. The residue was purified by flash silica gel chromatography (100-90% dichloromethane:methanol gradient @45 mL/min) to afford tert-butyl 4-[6-isopropoxy-5-(pyrazolo[1,5-a]pyrimidine-3-carbonylamino)pyrazolo[3,4-b]pyridin-2-yl]piperidine-1-carboxylate (445 mg, 98%) as yellow solid. MS (ESI) m/z: 521.3 [M+H]⁺.

Step 7: N-[6-isopropoxy-2-(4-piperidyl)pyrazolo[3,4-b]pyridin-5-yl]pyrazolo[1,5-a]pyrimidine-3-carboxamide

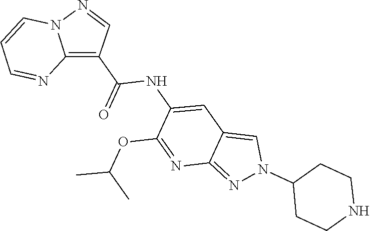 [SMILES: CC(C)Oc1nc2nn(C3CCNCC3)cc2cc1NC(=O)c1cnn2cccnc12]

To a solution of tert-butyl 4-[6-isopropoxy-5-(pyrazolo[1,5-a]pyrimidine-3-carbonylamino)pyrazolo[3,4-b]pyridin-2-yl]piperidine-1-carboxylate (440 mg, 0.9 mmol) in dichloromethane (5 mL) was added hydrogen chloride in methanol (4 M, 4 mL). The reaction mixture was stirred at 25° C. for 0.15 h, then concentrated under reduced pressure. Dichloromethane/methanol (10:1, 20 mL) was added to the residue, then added ammonium hydroxide to adjust the pH to 8-9, then concentrated. The residue was purified by column chromatography (petroleum ether:ethyl acetate=1:1 to dichloromethane:methanol=10:1) to afford N-[6-isopropoxy-2-(4-piperidyl)pyrazolo[3,4-b]pyridin-5-yl]pyrazolo[1,5-a]pyrimidine-3-carboxamide (370 mg, crude) as yellow solid. MS (ESI) m/z: 421.2[M+H]⁺; ¹H NMR (400 MHz, CDCl₃) δ 10.60 (s, 1H), 9.39 (dd, J=1.6, 7.2 Hz, 1H), 8.96 (s, 1H), 8.89 (dd, J=1.6, 4.4 Hz, 1H), 8.72 (s, 1H), 8.28 (s, 1H), 7.35 (dd, J=4.2, 7.0 Hz, 1H), 5.50-5.29 (m, 1H), 4.45-4.30 (m, 1H), 3.08 (d, J=12.4 Hz, 2H), 2.70-2.57 (m, 2H), 2.09-1.99 (m, 2H), 1.96-1.80 (m, 2H), 1.51 (d, J=6.0 Hz, 6H).

Step 8: tert-butyl 3-fluoro-3-[[4-[6-isopropoxy-5-(pyrazolo[1,5-a]pyrimidine-3-carbonylamino)pyrazolo[3,4-b]pyridin-2-yl]-1-piperidyl]methyl]azetidine-1-carboxylate

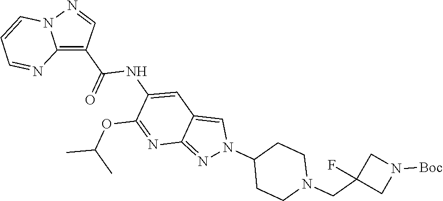 [SMILES: CC(C)Oc1nc2nn(C3CCN(CC4(F)CN(C(=O)OC(C)(C)C)C4)CC3)cc2cc1NC(=O)c1cnn2cccnc12]

To a solution of N-[6-isopropoxy-2-(4-piperidyl)pyrazolo[3,4-b]pyridin-5-yl]pyrazolo[1,5-a]pyrimidine-3-carboxamide (370 mg, 0.9 mmol) and tert-butyl 3-fluoro-3-(p-tolylsulfonyloxymethyl)azetidine-1-carboxylate (633 mg, 1.8 mmol) in dimethylsulfoxide (0.5 mL) was added N,N-diisopropylethylamine (569 mg, 4.4 mmol). The reaction mixture was stirred at 100° C. for 12 h. The reaction mixture was quenched by water (10 mL), then diluted with water (20 mL) and extracted with ethyl acetate (3×40 mL). The combined organic layers were washed with brine (3×60 mL), dried over anhydrous sodium sulfate, filtered, and concentrated under reduced pressure. The residue was purified by flash silica gel chromatography (0˜10% methanol:dichloromethanegradient @40 mL/min) to afford tert-butyl 3-fluoro-3-[[4-[6-isopropoxy-5-(pyrazolo[1,5-a]pyrimidine-3-carbonylamino)pyrazolo[3,4-b]pyridin-2-yl]-1-piperidyl]methyl]azetidine-1-carboxylate (500 mg, 93%) as yellow solid. MS (ESI) m/z: 608.3 [M+H]⁺; ¹H NMR (400 MHz, CDCl₃) δ 10.59 (s, 1H), 9.07 (s, 1H), 8.84 (dd, J=1.2, 6.8 Hz, 1H), 8.78 (s, 1H), 8.73 (dd, J=1.6, 4.0 Hz, 1H), 7.80 (s, 1H), 7.27 (s, 2H), 7.08 (dd, J=4.0, 7.2 Hz, 1H), 5.72-5.62 (m, 1H), 4.13-4.02 (m, 5H), 3.91 (d, J=6.4 Hz, 1H), 3.85 (d, J=6.0 Hz, 1H), 3.16-3.05 (m, 2H), 2.48-2.40 (m, 1H), 2.01 (t, J=6.0 Hz, 1H), 1.56 (d, J=6.0 Hz, 6H), 1.47 (s, 9H).

Step 9: N-[2-[1-[(3-fluoroazetidin-3-yl)methyl]-4-piperidyl]-6-isopropoxy-pyrazolo[3,4-b]pyridin-5-yl]pyrazolo[1,5-a]pyrimidine-3-carboxamide

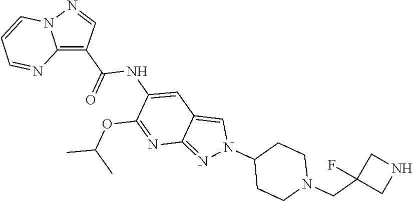 [SMILES: CC(C)Oc1nc2nn(C3CCN(CC4(F)CNC4)CC3)cc2cc1NC(=O)c1cnn2cccnc12]

To a solution of tert-butyl 3-fluoro-3-[[4-[6-isopropoxy-5-(pyrazolo[1,5-a]pyrimidine-3-carbonylamino)pyrazolo[3,4-b]pyridin-2-yl]-1-piperidyl]methyl]azetidine-1-carboxylate (150 mg, 0.3 mmol) in dichloromethane (2 mL) was added trifluoroacetic acid (924 mg, 8.1 mmol). The reaction mixture was stirred at 25° C. for 0.5 h, then concentrated under reduced pressure. Then to the residue was added dichloromethane (20 mL), ammonium hydroxide to adjust the pH to 8-9, then concentrated to afford N-[2-[1-[(3-fluoroazetidin-3-yl)methyl]-4-piperidyl]-6-isopropoxy-pyrazolo[3,4-b]pyridin-5-yl]pyrazolo[1,5-a]pyrimidine-3-carboxamide (125 mg, 99%) as yellow solid, which was used in the next step without further purification. MS (ESI) m/z: 508.2 [M+H]⁺.

Step 10: N-[2-[1-[[1-[2-(2,6-dioxo-3-piperidyl)-1-oxo-isoindolin-5-yl]-3-fluoro-azetidin-3-yl]methyl]-4-piperidyl]-6-isopropoxy-pyrazolo[3,4-b]pyridin-5-yl]pyrazolo[1,5-a]pyrimidine-3-carboxamide

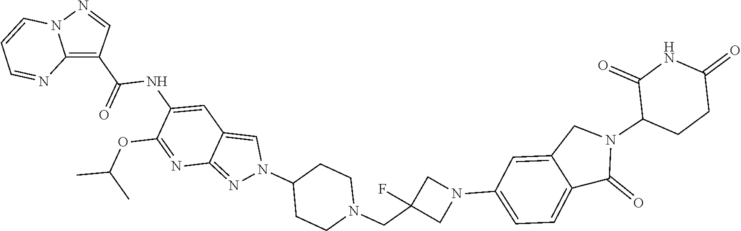 [SMILES: CC(C)Oc1nc2nn(C3CCN(CC4(F)CN(c5ccc6c(c5)CN(C5CCC(=O)NC5=O)C6=O)C4)CC3)cc2cc1NC(=O)c1cnn2cccnc12]

To a solution of N-[2-[1-[(3-fluoroazetidin-3-yl)methyl]-4-piperidyl]-6-isopropoxy-pyrazolo[3,4-b]pyridin-5-yl]pyrazolo[1,5-a]pyrimidine-3-carboxamide (250 mg, 0.5 mmol) and 3-(5-bromo-1-oxo-isoindolin-2-yl)piperidine-2,6-dione (159 mg, 0.5 mmol) in N,N-dimethylformamide (5 mL) was added cesium carbonate (401 mg, 1.2 mmol) and 1,3-bis[2,6-bis(1-ethylpropyl)phenyl]-2H-imidazole;3-chloropyridine;dichloropalladium (78 mg, 0.1 mmol). The reaction mixture was stirred at 80° C. for 3 h. The reaction mixture was filtered, then diluted with water (30 mL) and extracted with dichloromethane (3×30 mL). The combined organic layers were washed with brine (3×30 mL), dried over anhydrous sodium sulfate, filtered, and concentrated under reduced pressure. The residue was purified by prep-Thin layer chromatography (dichloromethane:methanol=10:1). The crude product was purified by prep-HPLC (column: Phenomenex Synergi C18 150*25 mm*10 um; mobile phase: [water (0.225% formic acid)-acetonitrile]; B %: 9%-39%, 10 min). Then the residue was further purified by prep-Thin layer chromatography (dichloromethane:methanol=10:1) to afford N-[2-[1-[[1-[2-(2,6-dioxo-3-piperidyl)-1-oxo-isoindolin-5-yl]-3-fluoro-azetidin-3-yl]methyl]-4-piperidyl]-6-isopropoxy-pyrazolo[3,4-b]pyridin-5-yl]pyrazolo[1,5-a]pyrimidine-3-carboxamide (73.2 mg, 19%) as white solid. MS (ESI) m/z: 750.2 [M+H]⁺; ¹H NMR (400 MHz, DMSO-d₆) δ 10.94 (s, 1H), 10.61 (s, 1H), 9.40 (dd, J=1.2, 6.8 Hz, 1H), 8.97 (s, 1H), 8.92-8.86 (m, 1H), 8.73 (s, 1H), 8.32 (s, 1H), 7.53 (d, J=8.4 Hz, 1H), 7.36 (dd, J=4.4, 6.8 Hz, 1H), 6.64 (s, 1H), 6.62-6.55 (m, 1H), 5.51-5.37 (m, 1H), 5.05 (dd, J=5.2, 13.2 Hz, 1H), 4.43-4.28 (m, 2H), 4.24-3.97 (m, 5H), 3.32-3.27 (m, 1H), 3.06 (d, J=11.6 Hz, 2H), 3.01-2.84 (m, 3H), 2.45-2.35 (m, 3H), 2.16-2.04 (m, 4H), 1.99-1.92 (m, 1H), 1.51 (d, J=6.0 Hz, 6H).

### Preparation of Example 417: N-(6-(cyclopropylmethoxy)-2-(4-(2-(2-(2,6-dioxopiperidin-3-yl)-1,3-dioxoisoindolin-5-yl)-2,6-diazaspiro[3.5]nonan-6-yl)cyclohexyl)-2H-indazol-5-yl)pyrazolo[1,5-a]pyrimidine-3-carboxamide

Step 1: 2-(4,4-dimethoxycyclohexyl)-5-nitro-2H-indazol-6-ol

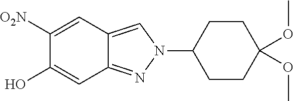 [SMILES: COC1(OC)CCC(n2cc3cc([N+](=O)[O-])c(O)cc3n2)CC1]

To a solution of 2-azido-4-hydroxy-5-nitro-benzaldehyde (2.09 g, 10.1 mmol) in toluene (10 mL) was added anhydrous sodium sulfate (21.41 g, 150.7 mmol, 15.29 mL) and 4,4-dimethoxycyclohexanamine (1.6 g, 10.1 mmol). The mixture was stirred under at 120° C. for 1 h, then filtered. The residue was purified by column chromatography (petroleum ether/ethyl acetate=10/1 tol/1) to afford 2-(4,4-dimethoxycyclohexyl)-5-nitro-2H-indazol-6-ol (2.1 g, 65%) as a yellow solid. MS (ESI) m/z: 289.8 [M+H]⁺; ¹H NMR (400 MHz, CDCl₃) δ 10.00 (s, 1H), 8.74 (s, 1H), 8.20 (s, 1H), 7.24 (s, 1H), 4.52-4.43 (m, 1H), 3.26 (s, 3H), 3.23 (s, 3H), 2.23 (br d, J=9.8 Hz, 4H), 2.19-2.05 (m, 2H), 1.67-1.55 (m, 2H).

Step 2: 6-(cyclopropylmethoxy)-2-(4,4-dimethoxycyclohexyl)-5-nitro-2H-indazole

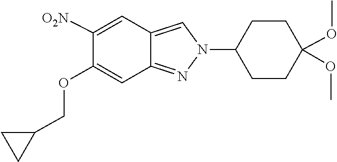 [SMILES: COC1(OC)CCC(n2cc3cc([N+](=O)[O-])c(OCC4CC4)cc3n2)CC1]

To a solution of bromomethylcyclopropane (8.82 g, 65.4 mmol, 6.26 mL) in N,N-dimethylformamide (20 mL) was added potassium carbonate (2.71 g, 19.6 mmol) and 2-(4,4-dimethoxycyclohexyl)-5-nitro-indazol-6-ol (2.1 g, 6.5 mmol). The mixture was stirred under at 60° C. for 2 h. The reaction mixture was filtered and concentrated under reduced pressure. The residue was purified by column chromatography (petroleum ether/ethyl acetate=10/1 tol/1) to afford 6-(cyclopropylmethoxy)-2-(4,4-dimethoxycyclohexyl)-5-nitro-indazole (2 g, 81%) as a yellow solid. MS (ESI) m/z: 344.1 [M+H]⁺; ¹H NMR (400 MHz, CDCl₃) δ 8.18 (s, 1H), 8.08 (s, 1H), 7.09 (s, 1H), 4.49-4.40 (m, 1H), 4.00-3.95 (m, 2H), 3.26 (s, 3H), 3.23 (s, 3H), 2.23-2.16 (m, 4H), 1.68-1.54 (m, 4H), 1.39-1.27 (m, 1H), 0.69-0.62 (m, 2H), 0.44-0.38 (m, 2H).

Step 3: 6-(cyclopropylmethoxy)-2-(4,4-dimethoxycyclohexyl)-2H-indazol-5-amine

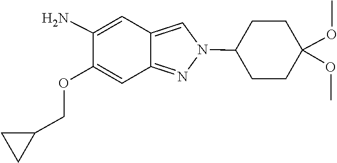 [SMILES: COC1(OC)CCC(n2cc3cc(N)c(OCC4CC4)cc3n2)CC1]

To a solution of 6-(cyclopropylmethoxy)-2-(4,4-dimethoxycyclohexyl)-5-nitro-indazole (1 g, 2.7 mmol) in 2,2,2-trifluoroethanol (10 mL) was added 10% palladium on carbon (1.5 g, 1.4 mmol) under nitrogen atmosphere. The suspension was degassed and purged with hydrogen atmosphere for 3 times. The mixture was stirred under hydrogen atmosphere (15 Psi) at 25° C. for 12 h, then filtered and concentrated under reduced pressure. The residue was purified by column chromatography (petroleum ether/ethyl acetate=10/1 tol/1) to afford 6-(cyclopropylmethoxy)-2-(4,4-dimethoxycyclohexyl)indazol-5-amine (0.7 g, 76%) as a yellow solid. MS (ESI) m/z: 314.1 [M+H]⁺; ¹H NMR (400 MHz, CHLOROFORM-d) δ 7.65 (s, 1H), 6.92 (s, 1H), 6.78 (s, 1H), 4.46-4.36 (m, 1H), 3.90 (d, J=6.8 Hz, 2H), 3.25 (s, 3H), 3.22 (s, 3H), 2.24-2.01 (m, 8H), 1.65-1.52 (m, 2H), 1.40-1.29 (m, 1H), 0.69-0.62 (m, 2H), 0.43-0.34 (m, 2H).

Step 4: 4-(5-amino-6-(cyclopropylmethoxy)-2H-indazol-2-yl)cyclohexanone

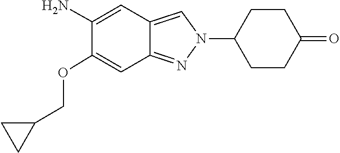 [SMILES: Nc1cc2cn(C3CCC(=O)CC3)nc2cc1OCC1CC1]

To a solution of 6-(cyclopropylmethoxy)-2-(4,4-dimethoxycyclohexyl)indazol-5-amine (1.3 g, 3.8 mmol) in tetrahydrofuran (20 mL) was added hydrogen chloride (5 M, 5 mL). The mixture was stirred under nitrogen at 25° C. for 12 h. The reaction mixture was concentrated in vacuo. The residue was purified by column chromatography (petroleum ether/ethyl acetate=10/1 tol/1) to afford 4-(5-amino-6-(cyclopropylmethoxy)-2H-indazol-2-yl)cyclohexanone (1 g, 88%) as a yellow solid. MS (ESI) m/z: 300.1 [M+H]⁺; ¹H NMR (400 MHz, CDCl₃) δ 7.65 (s, 1H), 6.90 (s, 1H), 6.78 (s, 1H), 4.84-4.74 (m, 1H), 3.90 (d, J=7.0 Hz, 2H), 2.69-2.38 (m, 10H), 1.40-1.30 (m, 1H), 0.70-0.62 (m, 2H), 0.43-0.36 (m, 2H).

Step 5: N-(6-(cyclopropylmethoxy)-2-(4-oxocyclohexyl)-2H-indazol-5-yl)pyrazolo[1,5-a]pyrimidine-3-carboxamide

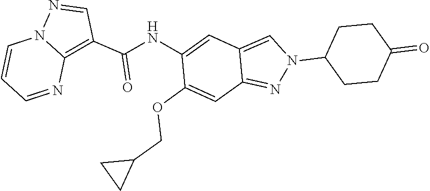 [SMILES: O=C1CCC(n2cc3cc(NC(=O)c4cnn5cccnc45)c(OCC4CC4)cc3n2)CC1]

To a solution of pyrazolo[1,5-a]pyrimidine-3-carboxylic acid (517.68 mg, 3.2 mmol) in pyridine (20 mL) was added N1-((ethylimino)methylene)-N3,N3-dimethylpropane-1,3-diamine hydrochloride (3.04 g, 15.9 mmol) and 4-[5-amino-6-(cyclopropylmethoxy)indazol-2-yl]cyclohexanone (950 mg, 3.2 mmol). The mixture was stirred under at 50° C. for 12 h, then concentrated in vacuo. The reaction was purified by column chromatography (petroleum ether/ethyl acetate=10/1 tol/1) to afford N-(6-(cyclopropylmethoxy)-2-(4-oxocyclohexyl)-2H-indazol-5-yl)pyrazolo[1,5-a]pyrimidine-3-carboxamide (1 g, 70%) as a yellow solid. MS (ESI) m/z: 445.2 [M+H]⁺; ¹H NMR (400 MHz, CDCl₃) δ 10.58 (s, 1H), 8.92 (s, 1H), 8.83 (dd, J=1.8, 7.0 Hz, 1H), 8.80 (s, 1H), 8.69 (dd, J=1.8, 4.0 Hz, 1H), 7.91 (s, 1H), 7.05 (dd, J=4.0, 7.0 Hz, 1H), 7.01 (s, 1H), 4.90-4.79 (m, 1H), 4.03 (d, J=6.8 Hz, 2H), 2.72-2.46 (m, 8H), 1.57-1.46 (m, 1H), 0.77-0.69 (m, 2H), 0.54 (q, J=4.8 Hz, 2H).

Step 6: tert-butyl 6-(4-(6-(cyclopropylmethoxy)-5-(pyrazolo[1,5-a]pyrimidine-3-carboxamido)-2H-indazol-2-yl)cyclohexyl)-2,6-diazaspiro[3.5]nonane-2-carboxylate

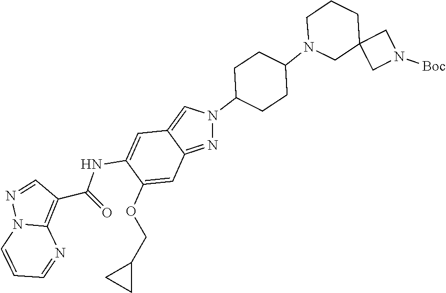 [SMILES: CC(C)(C)OC(=O)N1CC2(CCCN(C3CCC(n4cc5cc(NC(=O)c6cnn7cccnc67)c(OCC6CC6)cc5n4)CC3)C2)C1]

To a solution of N-[6-(cyclopropylmethoxy)-2-(4-oxocyclohexyl)indazol-5-yl]pyrazolo[1,5-a]pyrimidine-3-carboxamide (900 mg, 2.0 mmol), tert-butyl 2,8-diazaspiro[3.5]nonane-2-carboxylate (458.25 mg, 2.0 mmol) in 1,2-dichloroethane (9 mL), dimethylsulfoxide (9 mL), triethylamine (2.18 g, 21.6 mmol, 3 mL) was added sodium triacetoxyborohydride (858.28 mg, 4.1 mmol). The mixture was stirred under nitrogen at 25° C. for 12 h. The reaction mixture was diluted with water (10 mL). The organic phase was separated, washed with dichloromethane (3×10 mL), dried over anhydrous sodium sulfate, filtered, and concentrated under reduced pressure. The residue was purified by prep-Thin layer chromatography (column: Phenomenex luna C18 250*50 mm*10 um; mobile phase: [water (10 mM ammonium bicarbonate)-acetonitrile]; B %: 52 acetonitrile %-82 acetonitrile %, 23 min) to afford tert-butyl 6-(4-(6-(cyclopropylmethoxy)-5-(pyrazolo[1,5-a]pyrimidine-3-carboxamido)-2H-indazol-2-yl)cyclohexyl)-2,6-diazaspiro[3.5]nonane-2-carboxylate (250 mg, 19%) as a yellow solid. MS (ESI) m/z: 655.3 [M+H]⁺; ¹H NMR (400 MHz, DMSO) δ 10.64 (s, 1H), 9.37 (dd, J=1.6, 6.8 Hz, 1H), 8.87 (dd, J=1.6, 4.2 Hz, 1H), 8.73 (s, 1H), 8.71 (s, 1H), 8.25 (s, 1H), 7.32 (dd, J=4.0, 7.0 Hz, 1H), 7.03 (s, 1H), 4.37-4.27 (m, 1H), 4.01 (d, J=6.8 Hz, 2H), 3.17 (d, J=5.0 Hz, 1H), 2.46-2.23 (m, 11H), 1.97-1.82 (m, 4H), 1.43 (br d, J=4.6 Hz, 5H), 1.36 (s, 9H), 1.05 (t, J=7.0 Hz, 1H), 0.74-0.67 (m, 2H), 0.45 (q, J=4.6 Hz, 2H).

Step 7: N-(2-(4-(2,6-diazaspiro[3.5]nonan-6-yl)cyclohexyl)-6-(cyclopropylmethoxy)-2H-indazol-5-yl)pyrazolo[1,5-a]pyrimidine-3-carboxamide

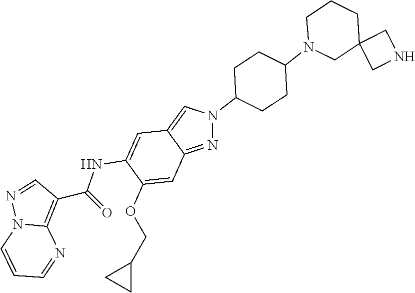 [SMILES: O=C(Nc1cc2cn(C3CCC(N4CCCC5(CNC5)C4)CC3)nc2cc1OCC1CC1)c1cnn2cccnc12]

To a solution of tert-butyl 6-(4-(6-(cyclopropylmethoxy)-5-(pyrazolo[1,5-a]pyrimidine-3-carboxamido)-2H-indazol-2-yl)cyclohexyl)-2,6-diazaspiro[3.5]nonane-2-carboxylate (250 mg, 381.8 umol) in dichloromethane (0.5 mL) was added trifluoroacetic acid (43.53 mg, 381.8 umol, 28.27 uL). The mixture was stirred at 25° C. for 0.5 h, then concentrated in vacuo to afford N-(2-(4-(2,6-diazaspiro[3.5]nonan-6-yl)cyclohexyl)-6-(cyclopropylmethoxy)-2H-indazol-5-yl)pyrazolo[1,5-a]pyrimidine-3-carboxamide trifluoroacetic acid (250 mg, 97%) as a yellow solid, which was used in the next step without further purification. MS (ESI) m/z: 555.1 [M+H]⁺.

Step 8: N-(6-(cyclopropylmethoxy)-2-(4-(2-(2-(2,6-dioxopiperidin-3-yl)-1,3-dioxoisoindolin-5-yl)-2,6-diazaspiro[3.5]nonan-6-yl)cyclohexyl)-2H-indazol-5-yl)pyrazolo[1,5-a]pyrimidine-3-carboxamide

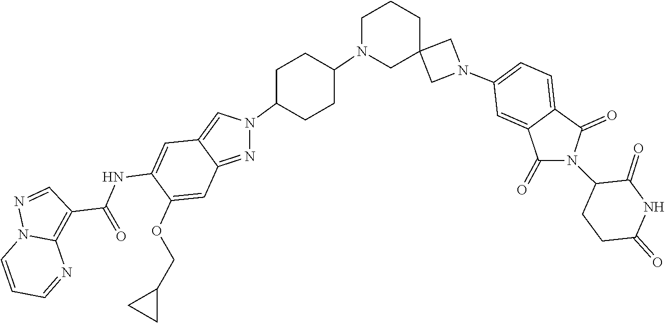 [SMILES: O=C1CCC(N2C(=O)c3ccc(N4CC5(CCCN(C6CCC(n7cc8cc(NC(=O)c9cnn%10cccnc9%10)c(OCC9CC9)cc8n7)CC6)C5)C4)cc3C2=O)C(=O)N1]

To a solution of N-(2-(4-(2,6-diazaspiro[3.5]nonan-6-yl)cyclohexyl)-6-(cyclopropylmethoxy)-2H-indazol-5-yl)pyrazolo[1,5-a]pyrimidine-3-carboxamide trifluoroacetic acid (250 mg, 373.9 umol), 2-(2,6-dioxo-3-piperidyl)-5-fluoro-isoindoline-1,3-dione (124 mg, 448.6 umol) in dimethylsulfoxide (2 mL) was added diisopropylethylamine (769.35 mg, 6.0 mmol, 1.04 mL). The mixture was stirred under at 100° C. for 12 h. The reaction mixture was quenched by water (20 mL), then extracted with dichloromethane (3×5 mL). The combined organic layers were washed with brine (3×10 mL), dried over anhydrous sodium sulfate, filtered, and concentrated under reduced pressure. The residue was purified by prep-Thin layer chromatography (column: Phenomenex Synergi C18 150*25 mm*10 um; mobile phase: [water(0.225% formic acid)-acetonitrile]; B %: 17%-41%, 8 min) to afford N-(6-(cyclopropylmethoxy)-2-(4-(2-(2-(2,6-dioxopiperidin-3-yl)-1,3-dioxoisoindolin-5-yl)-2,6-diazaspiro[3.5]nonan-6-yl)cyclohexyl)-2H-indazol-5-yl)pyrazolo[1,5-a]pyrimidine-3-carboxamide formic acid (122.2 mg, 38%) as a yellow solid. MS (ESI) m/z: 811.4 [M+H]⁺; ¹H NMR: (400 MHz, DMSO) δ 11.06 (d, J=4.4 Hz, 1H), 10.64 (d, J=5.8 Hz, 1H), 9.36 (d, J=7.0 Hz, 1H), 8.88-8.84 (m, 1H), 8.76-8.72 (m, 1H), 8.72-8.69 (m, 1H), 8.33-8.21 (m, 1H), 8.14 (s, 1H), 7.61 (dd, J=8.4, 11.2 Hz, 1H), 7.31 (ddd, J=1.8, 4.4, 6.6 Hz, 1H), 7.03 (d, J=12.2 Hz, 1H), 6.76 (br s, 1H), 6.64 (br d, J=8.2 Hz, 1H), 5.04 (td, J=6.4, 12.6 Hz, 1H), 4.53-4.26 (m, 1H), 4.05-3.97 (m, 2H), 3.80-3.64 (m, 6H), 2.94-2.80 (m, 2H), 2.62-2.52 (m, 4H), 2.39 (br d, J=7.4 Hz, 2H), 2.21-2.10 (m, 1H), 2.00 (br dd, J=5.2, 10.8 Hz, 1H), 1.95-1.81 (m, 4H), 1.63 (br s, 2H), 1.57-1.44 (m, 4H), 0.74-0.66 (m, 2H), 0.48-0.41 (m, 2H).

TABLE 15
The following compounds may be prepared in an analogous fashion as described for the
synthesis of Example 417.

| | | ¹H NMR |
|---|---|---|
| | | All spectra acquired at 400 MHz |
| Example | MS | unless otherwise indicated |
| Example 417 | See Above | |
| 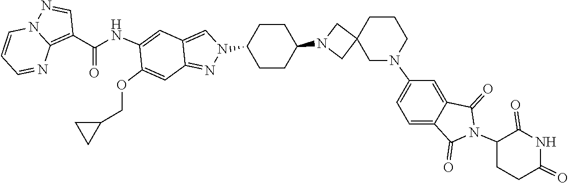 [SMILES: O=C1CCC(N2C(=O)c3ccc(N4CCCC5(C4)CN([C@H]4CC[C@H](n6cc7cc(NC(=O)c8cnn9cccnc89)c(OCC8CC8)cc7n6)CC4)C5)cc3C2=O)C(=O)N1] | 811.4 | DMSO-d₆ δ = 11.07 (s, 1H), 10.65 Hz, 1H), 7.37 (d, J = 2.0 Hz, 1H), 7.34-7.27 (m, 2H), 7.04 (s, 1H), 5.10 (dd, J = 5.4, 12.6 Hz, 1H), 4.37 (br s, 1H), 4.02 (d, J = 7.0 Hz, 2H), 3.60 (br d, J = 6.8 Hz, 2H), 3.01 (br d, J = 6.4 Hz, 2H), 2.95- 2.85 (m, 2H), 2.82 (br d, J = 6.8 Hz, 2H), 2.61 (br s, 1H), 2.59-2.53 (m, 2H), 2.42 (br d, J = 4.4 Hz, 1H), 2.29-2.18 (m, 2H), 2.08-1.98 (m, 1H), 1.83-1.73 (m, 4H), 1.70-1.57 |
| Example 418 | | (m, 4H), 1.57-1.45 (m, 3H), 0.75- |
| | | 0.67 (m, 2H), 0.49-0.42 (m, 2H) |
| 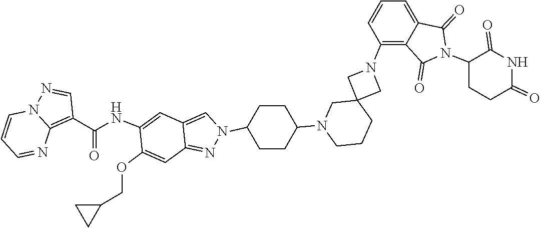 [SMILES: O=C1CCC(N2C(=O)c3cccc(N4CC5(CCCN(C6CCC(n7cc8cc(NC(=O)c9cnn%10cccnc9%10)c(OCC9CC9)cc8n7)CC6)C5)C4)c3C2=O)C(=O)N1] | 779.3 | DMSO-d₆ δ 11.09-11.03 (m, 1H), 10.67-10.61 (m, 1H), 9.37 (dd, J = 1.4, 7.0 Hz, 1H), 8.89 -8.83 (m, 1H), 8.77-8.67 (m, 2H), 8.33-8.13 (m, 2H), 7.58-7.49 (m, 1H), 7.31 (dd, J = 4.4, 6.8 Hz, 1H), 7.12-6.99 (m, 2H), 6.79 (d, J = 8.6 Hz, 1H), 5.04 (br dd, J = 5.4, 12.8 Hz, 1H), 4.38-4.25 (m, 1H), 4.03-3.99 (m, 2H), 3.96-3.80 (m, 4H), 2.92-2.82 (m, 1H), 2.65-2.52 (m, 5H), 2.16 (br d, J = 9.4 Hz, 2H), 2.04-1.82 (m, 6H), 1.66-1.42 (m, 8H), 0.70 (br d, J = 7.6 Hz, 2H), 0.45 (br d, J = 4.4 Hz, 2H) |
| Example 419 | | |

### Preparation of Example 420: N-[6-(cyclopropylmethoxy)-2-[1-[[1-[2-(2,6-dioxo-3-piperidyl)-7-fluoro-1-oxo-isoindolin-5-yl]-3-fluoro-azetidin-3-yl]methyl]-4-piperidyl]lindazol-5-yl]pyrazolo [1,5-a]pyrimidine-3-carboxamide

Step 1: methyl 4-bromo-2-fluoro-6-methyl-benzoate

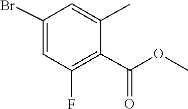

To a solution of 4-bromo-2-fluoro-6-methyl-benzoic acid (4.8 g, 20.6 mmol) and potassium carbonate (8.54 g, 61.8 mmol) in N,N-dimethylformamide (40 mL) was added methyl iodide (5.85 g, 41.2 mmol). The reaction was stirred at 80° C. for 1 h. The reaction mixture was quenched by water (10 mL), then diluted with water (20 mL) and extracted with ethyl acetate (3×60 mL). The combined organic layers were washed with brine (3×60 mL), dried over anhydrous sodium sulfate, filtered, and concentrated under reduced pressure. The residue was purified by flash silica gel chromatography (0˜10% petroleum ether:ethyl acetate gradient @40 mL/min) to afford methyl 4-bromo-2-fluoro-6-methyl-benzoate (5 g, 98%) as white solid. ¹H NMR (400 MHz, DMSO-d₆) δ 7.54 (d, J=9.2 Hz, 1H), 7.46 (s, 1H), 3.87 (s, 3H), 2.33 (s, 3H).

Step 2: methyl 4-bromo-2-(bromomethyl)-6-fluoro-benzoate

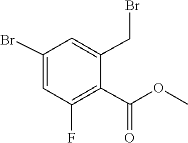

To a solution of methyl 4-bromo-2-fluoro-6-methyl-benzoate (100 mg, 0.4 mmol) and benzoyl peroxide (15 mg, 0.06 mmol) in carbon tetrachloride (10 mL) was added 1-bromopyrrolidine-2,5-dione (144 mg, 0.8 mmol). The reaction mixture was stirred at 95° C. for 12 h, then concentrated under reduced pressure. The residue was purified by prep-Thin layer chromatography (petroleum ether:ethyl acetate=30:1) to afford methyl 4-bromo-2-(bromomethyl)-6-fluoro-benzoate (30 mg, 22%) as colorless oil. ¹H NMR (400 MHz, CDCl₃) δ 7.40 (s, 1H), 7.29 (dd, J=1.6, 9.2 Hz, 1H), 4.61 (s, 2H), 3.98 (s, 3H).

Step 3: 3-(5-bromo-7-fluoro-1-oxo-isoindolin-2-yl)piperidine-2,6-dione

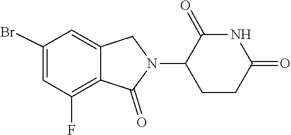 [SMILES: O=C1CCC(N2Cc3cc(Br)cc(F)c3C2=O)C(=O)N1]

To a solution of 3-aminopiperidine-2,6-dione hydrochloride (707 mg, 4.3 mmol) in N,N-dimethylformamide (10 mL) was added N,N-diisopropylethylamine (2.78 g, 21.5 mmol) and methyl 4-bromo-2-(bromomethyl)-6-fluoro-benzoate (1.4 g, 4.3 mmol). The reaction mixture was stirred at 80° C. for 12 h, then concentrated under reduced pressure. The residue was purified by re-crystallization from ethyl acetate (30 mL) which afforded 3-(5-bromo-7-fluoro-1-oxo-isoindolin-2-yl)piperidine-2,6-dione (1.05 g, 71%) as brown solid. MS (ESI) m/z: 343.0 [M+H]⁺; ¹H NMR (400 MHz, DMSO −d₆) δ 11.01 (s, 1H), 7.74 (s, 1H), 7.66 (d, J=9.2 Hz, 1H), 5.08 (dd, J=5.2, 13.2 Hz, 1H), 4.58-4.45 (m, 1H), 4.42-4.27 (m, 1H), 2.95-2.81 (m, 1H), 2.65-2.54 (m, 1H), 2.45-2.27 (m, 1H), 2.08-1.92 (m, 1H).

Step 4: N-[6-(cyclopropylmethoxy)-2-[1-[[1-[2-(2,6-dioxo-3-piperidyl)-7-fluoro-1-oxo-isoindolin-5-yl]-3-fluoro-azetidin-3-yl]methyl]-4-piperidyl]indazol-5-yl]pyrazolo[1,5-a]pyrimidine-3-carboxamide

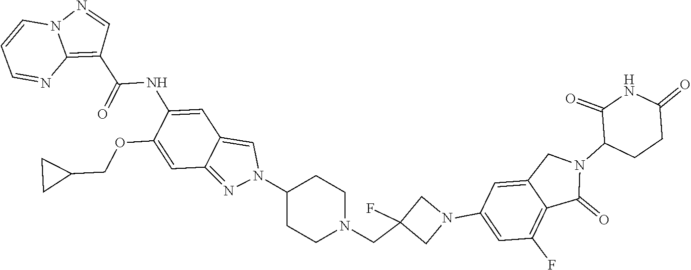 [SMILES: O=C1CCC(N2Cc3cc(N4CC(F)(CN5CCC(n6cc7cc(NC(=O)c8cnn9cccnc89)c(OCC8CC8)cc7n6)CC5)C4)cc(F)c3C2=O)C(=O)N1]

To a solution of 3-(5-bromo-7-fluoro-1-oxo-isoindolin-2-yl)piperidine-2,6-dione (230 mg, 0.67 mmol, 1 eq) and N-[6-(cyclopropylmethoxy)-2-[1-[(3-fluoroazetidin-3-yl)methyl]-4-piperidyl]indazol-5-yl]pyrazolo[1,5-a]pyrimidine-3-carboxamide (350 mg, 0.7 mmol) in N,N-dimethylformamide (5 mL) was added cesium carbonate (550 mg, 1.7 mmol) and 1,3-bis[2,6-bis(1-ethylpropyl)phenyl]-2H-imidazole;3-chloropyridine;dichloropalladium (107 mg, 0.1 mmol). The reaction was stirred at 80° C. for 3 h. The reaction mixture was filtered, then diluted with water (30 mL) and extracted with dichloromethane (3×30 mL). The combined organic layers were washed with brine (3×30 mL), dried over anhydrous sodium sulfate, filtered, and concentrated under reduced pressure. The residue was purified by prep-Thin layer chromatography (dichloromethane:methanol=10:1). Then the residue was further purified by prep-high performance liquid chromatography (column: Phenomenex luna C18 150*25 mm*10 um; mobile phase: [water (0.225% formic acid)-acetonitrile]; B %: 15%-45%, 10 min) to afford N-[6-(cyclopropylmethoxy)-2-[1-[[1-[2-(2,6-dioxo-3-piperidyl)-7-fluoro-1-oxo-isoindolin-5-yl]-3-fluoro-azetidin-3-yl]methyl]-4-piperidyl]indazol-5-yl]pyrazolo[1,5-a]pyrimidine-3-carboxamide (79.5 mg, 15%) as off-white solid. MS (ESI) m/z: 779.3[M+H]⁺; ¹H NMR (400 MHz, DMSO-d₆) δ 10.95 (s, 1H), 10.65 (s, 1H), 9.37 (dd, J=1.2, 6.8 Hz, 1H), 8.87 (dd, J=1.6, 4.4 Hz, 1H), 8.72 (d, J=10.8 Hz, 2H), 8.30 (s, 1H), 7.32 (dd, J=4.0, 6.8 Hz, 1H), 7.03 (s, 1H), 6.46 (s, 1H), 6.36 (d, J=11.6 Hz, 1H), 5.00 (dd, J=5.2, 13.2 Hz, 1H), 4.43-4.29 (m, 2H), 4.25-4.05 (m, 4H), 4.04-3.98 (in, 311), 3.11-3.01 (in, 211), 2.97 (s, 111), 2.93-2.82 (in, 211), 2.64-2.57 (in, 111), 2.43-2.29 (in, 311), 2.16-2.04 (in, 411), 2.00-1.90 (in, 111), 1.61-1.43 (in, 111), 0.81-0.63 (in, 211), 0.51-0.34 (in, 211).

TABLE 16
The following compounds may be prepared in an analogous fashion as described for the
synthesis of Example 420.

| | | ¹HNMR |
|---|---|---|
| | | All spectra acquired at 400 MHz |
| Example | MS | unless otherwise indicated |
| Example 420 | See Above | |
| 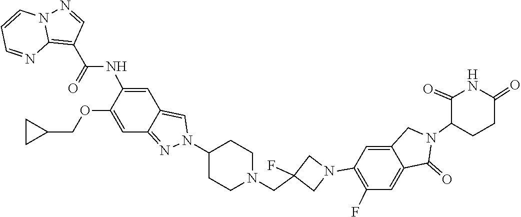 [SMILES: O=C1CCC(N2Cc3cc(N4CC(F)(CN5CCC(n6cc7cc(NC(=O)c8cnn9cccnc89)c(OCC8CC8)cc7n6)CC5)C4)c(F)cc3C2=O)C(=O)N1] | 779.32 | DMSO-d₆ δ 10.97 (br s, 1H), 10.66 (s, 1H), 9.39 (dd, J = 1.6, 7.2 Hz, 1H), 8.90-8.85 (m, 1H), 8.74 (d, J = 10.4 Hz, 2H), 8.32 (s, 1H), 8.28 (s, 1H), 7.39 (d, J = 11.6 Hz, 1H), 7.33 (dd, J = 4.4, 6.8 Hz, 1H), 7.04 (s, 1H), 6.82 (d, J = 7.6 Hz, 1H), 5.06 (dd, J = 5.2, 13.2 Hz, 1H), 4.45-4.36 (m, 1H), 4.33 (br d, J = 17.2 Hz, 1H), 4.29-4.25 (m, 1H), 4.23 (s, 1H), 4.20 (br d, J = 9.6 Hz, 1H), 4.18-4.08 (m, 2H), 4.03 (d, J = 6.8 Hz, 2H), 3.06 (br d, J = 10.8 Hz, 2H), 3.02-2.93 (m, 2H), 2.92-2.85 (m, 1H), 2.59 (br d, J = 17.2 Hz, 1H), 2.45-2.35 (m, 3H), 2.15- 2.05 (m, 4H), 2.02-1.94 (m, 1H), 1.55- |
| Example 421 | | 1.46 (m, 1H), 0.75-0.69 (m, 2H), 0.50-0.44 |
| | | (m, 2H) |

### Preparation of Example 422: N-{2-[1-({1-[2-(2,6-dioxopiperidin-3-yl)-1,3-dioxo-1H,2H,3H-pyrrolo[3,4-c]pyridin-6-yl]-3-fluoroazetidin-3-yl}methyl)piperidin-4-yl]-6-(propan-2-yloxy)-211-indazol-5-yl}pyrazolo[1,5-a]pyrimidine-3-carboxamide

Step 1: 6-chloropyridine-3,4-dicarboxylic acid

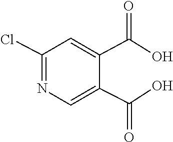 [SMILES: O=C(O)c1cnc(Cl)cc1C(=O)O]

To a solution of 2,2,6,6-tetramethylpiperidine (26.90 g, 190.4 mmol, 32.33 mL) in tetrahydrofuran (200 mL) was added n-butyllithium (2.5 M, 101.6 mL). 10 min later, 6-chloropyridine-3-carboxylic acid (10 g, 63.5 mmol) in tetrahydrofuran (50 mL) was added to the mixture at −50° C. Then 20 min later, the reaction mixture was stirred at −25° C. for 0.5 h. The solution was poured into dry carbon dioxide (200.00 g, 4.5 mol), the mixture was stirred for 0.5 h. The reaction mixture was quenched with water (200 mL), adjusted the pH to 2 with hydrochloric acid (2 M) at 0° C., extracted with dichloromethane (4×80 mL). The combined organic layers were washed with brine (80 mL), dried over anhydrous sodium sulfate, filtered, and concentrated. The crude product was triturated with ethyl acetate/acetonitrile/petroleum ether=1/1/2 (150 mL) at 25° C. for 10 min, filtered and dried to afford 6-chloropyridine-3,4-dicarboxylic acid (5 g, crude) as a brown solid. MS (ESI) m/z: 199.9 [M−1].

Step 2: dimethyl 6-chloropyridine-3,4-dicarboxylate

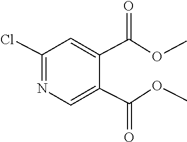

To a solution of 6-chloropyridine-3,4-dicarboxylic acid (3 g, 14.9 mmol) in dichloromethane (30 mL) and methyl alcohol (7 mL) was dropwsie added diazomethane (2 M, 22.50 mL) at 0° C., the mixture was stirred at 25° C. for 12 h. The mixture was poured into ice-water (20 mL). The aqueous phase was extracted with dichloromethane (3×20 mL). The combined organic phase was washed with brine (20 mL), dried over anhydrous sodium sulfate, filtered, and concentrated under vacuum. The residue was purified by silica gel column chromatography (petroleum ether/ethyl acetate=10/1 to 5/1). The crude product was purified by semi-preparative reverse phase HPLC (column: Phenomenex luna C18 150*40 mm*15 um; mobile phase: [water(0.225% formic acid)-acetonitrile]; B %: 27%-57%, 10 min) and then further purified by semi-preparative reverse phase HPLC (column: Waters Xbridge C18 150*50 mm*10 um; mobile phase: [water(10 mM ammonium bicarbonate)-acetonitrile]; B %: 20%-50%, 11 min) to afford dimethyl 6-chloropyridine-3,4-dicarboxylate (0.9 g, 26%) as a white solid. MS (ESI) m/z: 229.9 [M+H]⁺.

Step 3: dimethyl 6-(3-fluoro-3-((4-(6-isopropoxy-5-(pyrazolo[1,5-a]pyrimidine-3-carboxamido)-2H-indazol-2-yl)piperidin-1-yl)methyl)azetidin-1-yl)pyridine-3,4-dicarboxylate

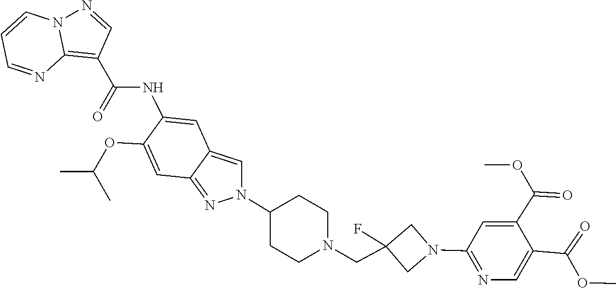 [SMILES: COC(=O)c1cnc(N2CC(F)(CN3CCC(n4cc5cc(NC(=O)c6cnn7cccnc67)c(OC(C)C)cc5n4)CC3)C2)cc1C(=O)OC]

To a solution of dimethyl 6-chloropyridine-3,4-dicarboxylate (0.2 g, 871.0 umol) and N-(2-(1-((3-fluoroazetidin-3-yl)methyl)piperidin-4-yl)-6-isopropoxy-2H-indazol-5-yl)pyrazolo[1,5-a]pyrimidine-3-carboxamide (441.23 mg, 871.0 umol) in dimethyl sulfoxide (0.5 mL) was added dropwsie N,N-diisopropylethylamine (2.97 g, 23.0 mmol, 4 mL) and the mixture was stirred at 100° C. for 12 h. The mixture was poured into ice-water (20 mL). The aqueous phase was extracted with dichloromethane (3×15 mL). The combined organic phase was washed with brine (3×10 mL), dried over anhydrous sodium sulfate, filtered, and concentrated under vacuum. The residue was purified by prep-Thin-layer chromatography (dichloromethane/methyl alcohol=10/1) to afford dimethyl 6-(3-fluoro-3-((4-(6-isopropoxy-5-(pyrazolo[1,5-a]pyrimidine-3-carboxamido)-2H-indazol-2-yl)piperidin-1-yl)methyl)azetidin-1-yl)pyridine-3,4-dicarboxylate (0.4 g, 65%) as a light yellow solid. MS (ESI) m/z: 700.3 [M+H]⁺.

Step 4: 6-(3-fluoro-3-((4-(6-isopropoxy-5-(pyrazolo[1,5-a]pyrimidine-3-carboxamido)-2H-indazol-2-yl)piperidin-1-yl)methyl)azetidin-1-yl)pyridine-3,4-dicarboxylic acid

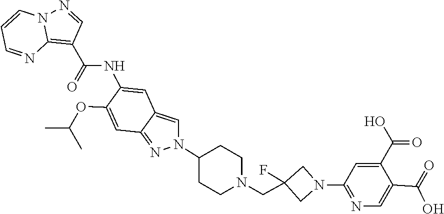 [SMILES: CC(C)Oc1cc2nn(C3CCN(CC4(F)CN(c5cc(C(=O)O)c(C(=O)O)cn5)C4)CC3)cc2cc1NC(=O)c1cnn2cccnc12]

To a solution of dimethyl 6-(3-fluoro-3-((4-(6-isopropoxy-5-(pyrazolo[1,5-a]pyrimidine-3-carboxamido)-2H-indazol-2-yl)piperidin-1-yl)methyl)azetidin-1-yl)pyridine-3,4-dicarboxylate (0.4 g, 571.7 umol) in water (0.8 mL) and tetrahydrofuran (0.8 mL) was added lithium hydroxide monohydrate (80.00 mg, 1.9 mmol), then the mixture was stirred at 25° C. for 12 h. The mixture was concentrated under vacuum, adjusted the pH to 6 with formic acid. The residue was purified by semi-preparative reverse phase HPLC (column: Phenomenex luna C18 150*40 mm*um; mobile phase: [water(formic acid)-acetonitrile]; B %: 15%-45%, 10 min) to afford 6-(3-fluoro-3-((4-(6-isopropoxy-5-(pyrazolo[1,5-a]pyrimidine-3-carboxamido)-2H-indazol-2-yl)piperidin-1-yl)methyl)azetidin-1-yl)pyridine-3,4-dicarboxylic acid (0.25 g, 65%) as an off-white solid. MS (ESI) m/z: 672.3 [M+H]⁺.

Step 5: N-(2-(1-((1-(2-(2,6-dioxopiperidin-3-yl)-1,3-dioxo-2,3-dihydro-1H-pyrrolo[3,4-c]pyridin-6-yl)-3-fluoroazetidin-3-yl)methyl)piperidin-4-yl)-6-isopropoxy-2H-indazol-5-yl)pyrazolo[1,5-a]pyrimidine-3-carboxamide

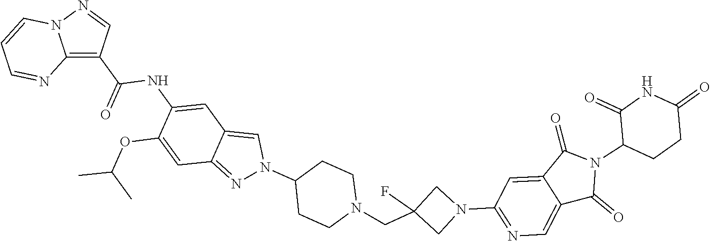 [SMILES: CC(C)Oc1cc2nn(C3CCN(CC4(F)CN(c5cc6c(cn5)C(=O)N(C5CCC(=O)NC5=O)C6=O)C4)CC3)cc2cc1NC(=O)c1cnn2cccnc12]

To a solution of 3-aminopiperidine-2,6-dione hydrochloride (500.00 mg, 3.0 mmol) in acetic acid (8 mL) was added sodium acetate (333.33 mg, 4.1 mmol) and stirred at 25° C. for 0.5 h, then 6-(3-fluoro-3-((4-(6-isopropoxy-5-(pyrazolo[1,5-a]pyrimidine-3-carboxamido)-2H-indazol-2-yl)piperidin-1-yl)methyl)azetidin-1-yl)pyridine-3,4-dicarboxylic acid (0.5 g, 744.4 umol) was added. The resulting the mixture was stirred at 100° C. for 11.5 h, then concentrated under vacuum. The residue was diluted with dichloromethane (30 mL) and then adjusted the pH to 8 with saturated sodium bicarbonate at 0° C. The resulting suspension was filtered and washed with dichloromethane (2×15 mL). The filtrate was extracted with dichloromethane (3×20 mL). The combined organic phase was washed with brine (2×10 mL), dried over anhydrous sodium sulfate, filtered, and concentrated under vacuum. The residue was purified by semi-preparative reverse phase HPLC (column: Phenomenex Synergi C18 150*25 mm*10 um; mobile phase: [water(formic acid)-dichloromethane]; B %: 11%-41%, 10 min) to afford N-(2-(1-((1-(2-(2,6-dioxopiperidin-3-yl)-1,3-dioxo-2,3-dihydro-1H-pyrrolo[3,4-c]pyridin-6-yl)-3-fluoroazetidin-3-yl)methyl)piperidin-4-yl)-6-isopropoxy-2H-indazol-5-yl)pyrazolo[1,5-a]pyrimidine-3-carboxamide (63.5 mg, 10%) as a yellow solid. MS (ESI) m/z: 764.3 [M+H]; ¹H NMR: (400 MHz, DMSO-d₆) δ 11.11 (s, 1H) 10.59 (s, 1H) 9.38 (dd, J=6.8, 1.2 Hz, 1H) 8.87 (dd, J=4.0, 1.2 Hz, 1H) 8.74 (s, 1H) 8.71 (s, 1H) 8.62 (s, 1H) 8.30 (s, 1H) 7.34 (dd, J=6.8, 4.0 Hz, 1H) 7.13 (s, 1H) 6.95 (s, 1H) 5.12 (dd, J=12.8, 5.6 Hz, 1H) 4.85 (quin, J=6.0 Hz, 1H) 4.24-4.44 (m, 5H) 2.83-3.09 (m, 5H) 2.53-2.69 (m, 2H) 2.43 (br s, 2H) 2.00-2.15 (m, 5H) 1.47 (d, J=6.0 Hz, 6H).

### Preparation of Example 423: N-[6-(cyclopropylmethoxy)-2-[(1r,4r)-4-({1-[2-(2,6-dioxopiperidin-3-yl)-1-oxo-2,3-dihydro-1H-isoindol-5-yl]azetidin-3-yl}oxy)cyclohexyl]-2H-indazol-5-yl]pyrazolo[1,5-a]pyrimidine-3-carboxamide

Step 1: diethyl 2-(((1r,4r)-4-(6-hydroxy-5-nitro-2H-indazol-2-yl)cyclohexyl)oxy)malonate

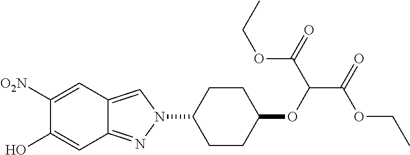

To a solution of diethyl 2-(((1r,4r)-4-aminocyclohexyl)oxy)malonate (10 g, 36.6 mmol) in toluene (300 mL) was added 2-azido-4-hydroxy-5-nitrobenzaldehyde (7.61 g, 36.6 mmol) and anhydrous sodium sulfate (103.94 g, 731.7 mmol, 74.24 mL). The reaction mixture was stirred at 25° C. for 10 min and then heated to 120° C. for 20 min. The mixture was concentrated under vacuum. The residue was purified by silica gel column chromatography (petroleum ether/ethyl acetate=5/1 to 2/1) to afford diethyl 2-(((1r,4r)-4-(6-hydroxy-5-nitro-2H-indazol-2-yl)cyclohexyl)oxy)malonate (3.6 g, 21%) as a red solid. ¹H NMR (400 MHz, CDCl₃) δ 9.98 (s, 1H) 8.73 (s, 1H) 8.15 (d, J=0.8 Hz, 1H) 7.21 (s, 1H) 4.62 (s, 1H) 4.42 (m, 1H) 4.25-4.34 (m, 4H) 3.62 (m, 1H) 2.26-2.38 (m, 4H) 1.98-2.09 (m, 2H) 1.63-1.71 (m, 2H) 1.32 (t, J=7.2 Hz, 6H).

Step 2: diethyl 2-(((1r,4r)-4-(6-(cyclopropylmethoxy)-5-nitro-2H-indazol-2-yl)cyclohexyl)oxy)malonate

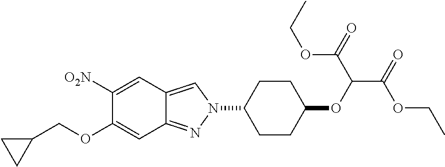

To a solution of diethyl 2-(((1r,4r)-4-(6-hydroxy-5-nitro-2H-indazol-2-yl)cyclohexyl)oxy)malonate (15 g, 34.5 mmol) in N,N-dimethylformamide (150 mL) was added potassium carbonate (14.28 g, 103.4 mmol) and bromomethylcyclopropane (13.95 g, 103.4 mmol, 9.90 mL). The mixture was stirred at 60° C. for 2 h, then concentrated in vacuo. The residue was purified by column chromatography (silica gel, petroleum ether/ethyl acetate=5/1 to 0/1) to afford diethyl 2-(((1r,4r)-4-(6-(cyclopropylmethoxy)-5-nitro-2H-indazol-2-yl)cyclohexyl)oxy)malonate (12 g, 71%) as a brown solid. MS (ESI) m/z: 490.2 [M+H]⁺.

Step 3: 2-(((1r,4r)-4-(6-(cyclopropylmethoxy)-5-nitro-2H-indazol-2-yl)cyclohexyl)oxy)malonic acid

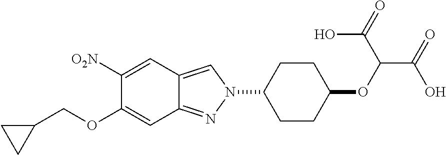

To a solution of diethyl 2-(((1r,4r)-4-(6-(cyclopropylmethoxy)-5-nitro-2H-indazol-2-yl)cyclohexyl)oxy)malonate (12 g, 24.6 mmol) in tetrahydrofuran (40 mL), methanol (40 mL) and water (40 mL) was added lithium hydroxide (3.10 g, 73.8 mmol). The mixture was stirred at 25° C. for 2 h, then concentrated in vacuo. The crude product was purified by preparative HPLC (column: Phenomenex luna C18 (250*70 mm, 10 um); mobile phase: [water(formic acid)-acetonitrile]; B %: 28%-58%, 20 min) to afford 2-(((1r,4r)-4-(6-(cyclopropylmethoxy)-5-nitro-2H-indazol-2-yl)cyclohexyl)oxy)malonic acid (8.6 g, 81%) as a yellow solid. MS (ESI) m/z: 434.1 [M+H]⁺.

Step 4: 2-(((1r,4r)-4-(6-(cyclopropylmethoxy)-5-nitro-2H-indazol-2-yl)cyclohexyl)oxy)propane-1,3-diol

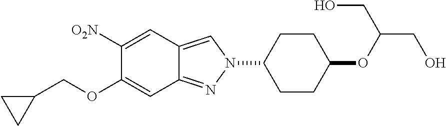

To a solution of 2-(((1r,4r)-4-(6-(cyclopropylmethoxy)-5-nitro-2H-indazol-2-yl)cyclohexyl)oxy)malonic acid (8.6 g, 19.8 mmol) in tetrahydrofuran (200 mL) was added borane dimethyl sulfide complex (10 M, 4.96 mL). The mixture was stirred at 25° C. for 12 h, then concentrated in vacuo. The residue was purified by column chromatography (petroleum ether/ethyl acetate=20/1 to 0/1) to afford 2-(((1r,4r)-4-(6-(cyclopropylmethoxy)-5-nitro-2H-indazol-2-yl)cyclohexyl)oxy)propane-1,3-diol (7 g, 87%) as a white solid. MS (ESI) m/z: 405.9 [M+H]⁺.

Step 5: 2-(((1r,4r)-4-(6-(cyclopropylmethoxy)-5-nitro-2H-indazol-2-yl)cyclohexyl)oxy)propane-1,3-diyl dimethanesulfonate

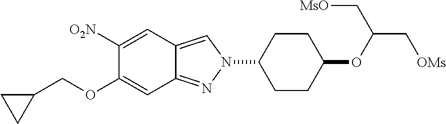

To a solution of 2-(((1r,4r)-4-(6-(cyclopropylmethoxy)-5-nitro-2H-indazol-2-yl)cyclohexyl)oxy)propane-1,3-diol (7 g, 17.3 mmol) in dichloromethane (100 mL) was added triethylamine (10.48 g, 103.6 mmol, 14.42 mL) and methanesulfonyl chloride (9.34 g, 81.5 mmol, 6.31 mL). The mixture was stirred at 25° C. for 1 h, then concentrated in vacuo. The residue was purified by column chromatography (petroleum ether/ethyl acetate=20/1 to 0/1) to afford 2-(((1r,4r)-4-(6-(cyclopropylmethoxy)-5-nitro-2H-indazol-2-yl)cyclohexyl)oxy)propane-1,3-diyl dimethanesulfonate (9 g, 93%) as a white solid. MS (ESI) m/z: 562.0 [M+H]⁺.

Step 6: 6-(cyclopropylmethoxy)-2-((1r,4r)-4-((1-(2,4-dimethoxybenzyl)azetidin-3-yl)oxy)cyclohexyl)-5-nitro-2H-indazole

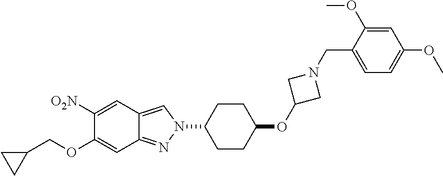

To a solution of 2-(((1r,4r)-4-(6-(cyclopropylmethoxy)-5-nitro-2H-indazol-2-yl)cyclohexyl)oxy)propane-1,3-diyl dimethanesulfonate (9 g, 16.0 mmol) in dimethylsulfoxide (100 mL) was added N,N-diisopropylethylamine (6.21 g, 48.1 mmol, 8.37 mL) and (2,4-dimethoxyphenyl)methanamine (5.36 g, 32.1 mmol, 4.83 mL). The mixture was stirred at 80° C. for 12 h. To the reaction mixture was added water (300 mL) and extracted with ethyl acetate (3×300 mL). The combined organic phase was washed with brine (200 mL), dried over anhydrous sodium sulfate, filtered, and concentrated in vacuo. The residue was purified by column chromatography (petroleum ether/ethyl acetate=20/1 to 0/1) to afford 6-(cyclopropylmethoxy)-2-((1r,4r)-4-((1-(2,4-dimethoxybenzyl)azetidin-3-yl)oxy)cyclohexyl)-5-nitro-2H-indazole (8 g, 93%) as a white solid. MS (ESI) m/z: 537.1 [M+H]⁺.

Step 7: 1-(3-(((1r,4r)-4-(6-(cyclopropylmethoxy)-5-nitro-2H-indazol-2 yl)cyclohexyl)oxy)azetidin-1-yl)-2,2,2-trifluoroethan-1-one

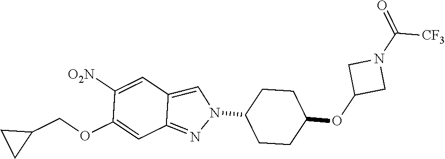

To a solution of 6-(cyclopropylmethoxy)-2-((1r,4r)-4-((1-(2,4-dimethoxybenzyl)azetidin-3-yl)oxy)cyclohexyl)-5-nitro-2H-indazole (8 g, 14.9 mmol) in dichloromethane (80 mL) was added N,N-diisopropylethylamine (30.17 g, 298.2 mmol, 41.50 mL) and trifluoroacetic anhydride (31.31 g, 149.1 mmol, 20.74 mL). The mixture was stirred at 25° C. for 1 h. To the reaction mixture was added water (100 mL) and extracted with ethyl acetate (3×100 mL). The combined organic phase was washed with brine (100 mL), dried over anhydrous sodium sulfate, filtered, and concentrated in vacuo. The residue was purified by column chromatography (petroleum ether/ethyl acetate=10/1 to 1/1) to afford 1-(3-(((1r,4r)-4-(6-(cyclopropylmethoxy)-5-nitro-2H-indazol-2-yl)cyclohexyl)oxy)azetidin-1-yl)-2,2,2-trifluoroethan-1-one (5.8 g, 81%) as a white solid. MS (ESI) m/z: 482.9 [M+H]⁺.

Step 8: 1-(3-(((1r,4r)-4-(5-amino-6-(cyclopropylmethoxy)-2H-indazol-2-yl)cyclohexyl)oxy)azetidin-1-yl)-2,2,2-trifluoroethan-1-one

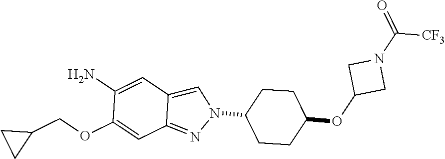

To a solution of 1-(3-(((1r,4r)-4-(6-(cyclopropylmethoxy)-5-nitro-2H-indazol-2-yl)cyclohexyl)oxy)azetidin-1-yl)-2,2,2-trifluoroethan-1-one (5.8 g, 12.0 mmol) in trifluoroethanol (100 mL) was added 10% palladium/carbon (2 g) under nitrogen. The suspension was degassed under vacuum and purged with hydrogen several times. The mixture was stirred under hydrogen (15 psi) at 25° C. for 0.5 h. The reaction mixture was filtered and the filtrate was concentrated. The residue was purified by column chromatography (petroleum ether/ethyl acetate=10/1 to 1/1) to afford 1-(3-(((1r,4r)-4-(5-amino-6-(cyclopropylmethoxy)-2H-indazol-2-yl)cyclohexyl)oxy)azetidin-1-yl)-2,2,2-trifluoroethan-1-one (5.4 g, 99%) as a white solid. MS (ESI) m/z: 452.9 [M+H]⁺.

Step 9: N-(6-(cyclopropylmethoxy)-2-((1r,4r)-4-((1-(2,2,2-trifluoroacetyl)azetidin-3-yl)oxy)cyclohexyl)-2H-indazol-5-yl)pyrazolo[1,5-a]pyrimidine-3-carboxamide

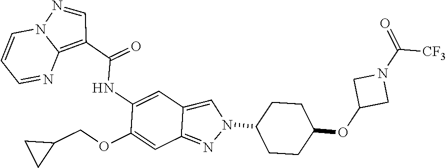

To a solution of 1-(3-(((1r,4r)-4-(5-amino-6-(cyclopropylmethoxy)-2H-indazol-2-yl)cyclohexyl)oxy)azetidin-1-yl)-2,2,2-trifluoroethan-1-one (5.4 g, 11.9 mmol) pyrazolo[1,5-a]pyrimidine-3-carboxylic acid (1.95 g, 11.9 mmol) in pyridine (100 mL) was added 1-(3-dimethylaminopropyl)-3-ethylcarbodiimide hydrochloride (4.58 g, 23.9 mmol). The mixture was stirred at 50° C. for 1 h, then concentrated in vacuo. The residue was purified by column chromatography (dichloromethane/methanol=30/1 to 10:1) to afford N-(6-(cyclopropylmethoxy)-2-((1r,4r)-4-((1-(2,2,2-trifluoroacetyl)azetidin-3-yl)oxy)cyclohexyl)-2H-indazol-5-yl)pyrazolo[1,5-a]pyrimidine-3-carboxamide (5 g, 70%) as a white solid. MS (ESI) m/z: 598.2 [M+H]⁺.

Step 10: N-(2-((1r,4r)-4-(azetidin-3-yloxy)cyclohexyl)-6-(cyclopropylmethoxy)-2H-indazol-5-yl)pyrazolo[1,5-a]pyrimidine-3-carboxamide

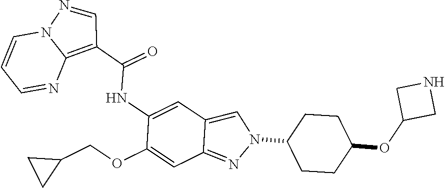

To a solution of N-(6-(cyclopropylmethoxy)-2-((1r,4r)-4-((1-(2,2,2-trifluoroacetyl)azetidin-3-yl)oxy)cyclohexyl)-2H-indazol-5-yl)pyrazolo[1,5-a]pyrimidine-3-carboxamide (1 g, 1.7 mmol) in methanol (10 mL) tetrahydrofuran (10 mL) water (10 mL) was added lithium hydroxide (120.23 mg, 5.0 mmol). The mixture was stirred at 25° C. for 1 h, then concentrated in vacuo. The crude product was purified by preparative HPLC (column: Waters Xbridge C18 150*50 mm*10 um; mobile phase: [water(sodium bicarbonate)-acetonitrile]; B %: 25%-55%, 11 min) to afford N-(2-((1r,4r)-4-(azetidin-3-yloxy)cyclohexyl)-6-(cyclopropylmethoxy)-2H-indazol-5-yl)pyrazolo[1,5-a]pyrimidine-3-carboxamide (300 mg, 36%) as a white solid. MS (ESI) m/z: 502.2 [M+H]⁺.

Step 11: N-(6-(cyclopropylmethoxy)-2-((1r,4r)-4-((1-(2-(2,6-dioxopiperidin-3-yl)-1-oxoisoindolin-5-yl)azetidin-3-yl)oxy)cyclohexyl)-2H-indazol-5-yl)pyrazolo[1,5-a]pyrimidine-3-carboxamide

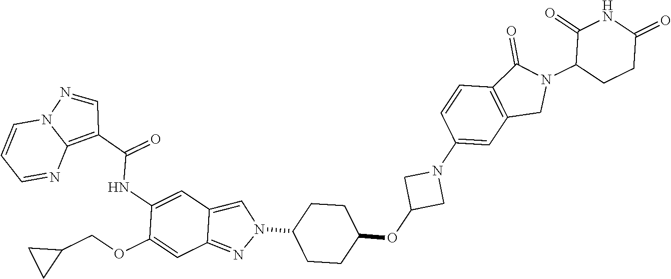

A mixture of N-(2-((1r,4r)-4-(azetidin-3-yloxy)cyclohexyl)-6-(cyclopropylmethoxy)-2H-indazol-5-yl)pyrazolo[1,5-a]pyrimidine-3-carboxamide (200 mg, 398.7 umol), 3-(5-bromo-1-oxo-isoindolin-2-yl)piperidine-2,6-dione (128.85 mg, 398.7 umol), 1,3-bis[2,6-bis(1-ethylpropyl)phenyl]-2H-imidazole;3-chloropyridine;dichloropalladium (126.59 mg, 159.5 umol), cesium carbonate (324.79 mg, 996.8 umol) in N,N-dimethylformamide (8 mL) was degassed and purged with nitrogen for 3 times, then the mixture was stirred at 80° C. for 3 h under nitrogen atmosphere. The reaction was filtered, and the filtrate was concentrated in vacuo. The crude product was purified by preparative HPLC (column: Phenomenex Synergi C18 150*25 mm*10 um; mobile phase: [water(formic acid)-acetonitrile]; B %: 36%-63%, 9 min) to afford N-(6-(cyclopropylmethoxy)-2-((1r,4r)-4-((1-(2-(2,6-dioxopiperidin-3-yl)-1-oxoisoindolin-5-yl)azetidin-3-yl)oxy)cyclohexyl)-2H-indazol-5-yl)pyrazolo[1,5-a]pyrimidine-3-carboxamide (17.6 mg, 6%) as an off-white solid. MS (ESI) m/z: 744.3 [M+H]⁺; 1H NMVR (400 MHllz, CDCl₃) δ 10.94 (s, 1H), 10.64 (s, 1H), 9.36-9.40 (m, 1H), 8.85-8.89 (m, 1H), 8.74 (s, 1H), 8.71 (s, 1H), 8.26 (s, 1H), 7.50 (d, J=8.4 Hz, 1H), 7.32 (m, 1H), 7.03 (s, 1H), 6.55 (s, 1H), 6.51 (d, J=8.8 Hz, 1H), 5.04 (m, 1H), 4.63 (s, 1H), 4.40 (d, J=7.6 Hz, 1H), 4.13-4.31 (m, 4H), 4.02 (d, J=7.2 Hz, 2H), 3.71 (s, 2H), 3.52 (d, J=10.0 Hz, 1H), 2.83-2.96 (m, 1H), 2.12 (s, 4H), 1.95 (d, J=11.62 Hz, 4H), 1.35-1.57 (m, 4H), 0.66-0.75 (m, 2H), 0.46 (d, J=4.4 Hz, 2H).

TABLE 17
The following compounds may be prepared in an analogous fashion as described for the
synthesis of Example 423.

| | | ¹H NMR |
|---|---|---|
| | | All spectra acquired at 400 MHz |
| Example | MS | unless otherwise indicated |
| Example 423 | See Above | |
| 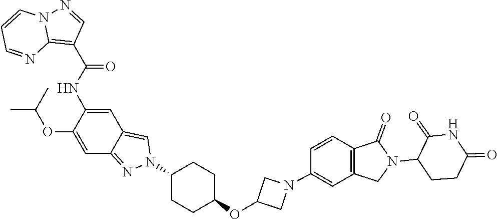 [SMILES: CC(C)Oc1cc2nn([C@H]3CC[C@H](OC4CN(c5ccc6c(c5)CN(C5CCC(=O)NC5=O)C6=O)C4)CC3)cc2cc1NC(=O)c1cnn2cccnc12] | Not Determined | |
| Example 424 | | |
| 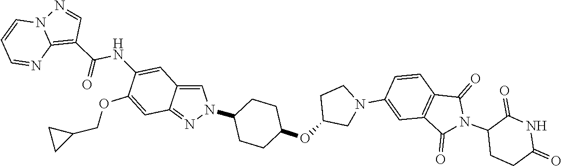 [SMILES: O=C1CCC(N2C(=O)c3ccc(N4CC[C@@H](O[C@H]5CC[C@@H](n6cc7cc(NC(=O)c8cnn9cccnc89)c(OCC8CC8)cc7n6)CC5)C4)cc3C2=O)C(=O)N1] | 772.3 | DMSO-d₆ δ 11.07 (br s, 1 H) 10.63 (s, 1 H) 9.37 (dd, J = 6.8, 1.6 Hz, 1 H) 8.86 (dd, J=4.0, 1.2 Hz, 1 H) 8.72 (d, J = 8.8 Hz, 2 H) 8.48 (s, 1 H) 8.26 (s, 1 H) 7.65 (d, J = 8.4 Hz, 1 H) 7.28- 7.36 (m, 1 H) 7.05 (s, 1 H) 6.96 (d, J = 2.0 Hz, 1 H) 6.79-6.89 (m, 1 H) 5.06 (dd, J = 12.8, 5.2 Hz, 1 H) 4.34-4.45 (m, 2 H) 4.00 (d, J = 6.8 Hz, 2 H) 3.78 (br s, 1 H) 3.64 (br dd, |
| Example 425 | | J = 11.2,4.8 Hz, 1 H) 3.50 (br t, J = |
| | | 6.8 Hz, 2 H) 3.43 (br d, J = 11.6 Hz, |
| | | 1H) 2.82-2.97 (m, 1 H) 2.55-2.69 (m, |
| | | 2 H) 2.10-2.23 (m, 4 H) 1.84-2.04 |
| | | (m, 5 H) 1.56-1.72 (m, 2 H) 1.44- |
| | | 1.54 (m, 1 H) 0.66-0.73 |
| | | (m, 2 H) 0.41-0.48 (m, 2 H) |
| 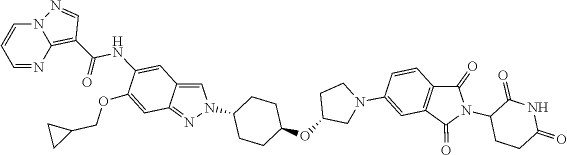 [SMILES: O=C1CCC(N2C(=O)c3ccc(N4CC[C@@H](O[C@H]5CC[C@H](n6cc7cc(NC(=O)c8cnn9cccnc89)c(OCC8CC8)cc7n6)CC5)C4)cc3C2=O)C(=O)N1] | 772.2 | DMSO-d₆ δ 11.07 (s, 1 H) 10.64 (s, 1 H) 9.37 (dd, J = 6.8, 1.6 Hz, 1 H) 8.86 (dd, J = 4.4, 1.6 Hz, 1 H) 8.72 (d, J = 10.8 Hz, 2 H) 8.48 (s, 1 H) 8.25 (s, 1 H) 7.65 (d, J = 8.4 Hz, 1 H) 7.28-7.34 (m, 1 H) 7.02 (s, 1 H) 6.94 (d, J = 2.0 Hz, 1 H) 6.84 (dd, J = 8.8, 2.4 Hz, 1 H) 5.06 (dd, J = 12.8, 5.6 Hz, 1 H) 4.34-4.49 (m, 2 H) 4.02 (d, |
| Example 426 | | J = 7.2 Hz, 2 H) 3.63 (br dd, |
| | | J = 11.2, 4.8 Hz, 1 H) 3.51-3.59 |
| | | (m, 1 H) 3.48 (br t, J = 7.2 Hz, 2 |
| | | H) 3.40 (br d, J = 10.8 Hz, 2 H) |
| | | 2.83-2.96 (m, 1 H) 2.54-2.63 (m, 2 |
| | | H) 2.07-2.17 (m, 6 H) 1.90-2.03 (m, |
| | | 2 H) 1.35-1.53 (m, 3 H) 0.66-0.74 |
| | | (m, 2 H) 0.42-0.49 (m, 2 H) |
| 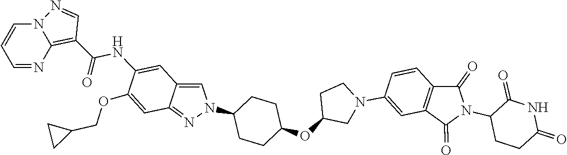 [SMILES: O=C1CCC(N2C(=O)c3ccc(N4CC[C@H](O[C@H]5CC[C@@H](n6cc7cc(NC(=O)c8cnn9cccnc89)c(OCC8CC8)cc7n6)CC5)C4)cc3C2=O)C(=O)N1] | 772.2 | DMSO-d₆ δ 11.07 (s, 1H), 10.63 (s, 1H), 9.46-9.30 (m, 1H), 8.93-8.82 (m, 1H), 8.72 (d, J = 8.4 Hz, 2H), 8.26 (s, 1H), 7.65 (d, J = 8.4 Hz, 1H), 7.31 (dd, J = 4.4, 6.8 Hz, 1H), 7.05 (s, 1H), 6.96 (s, 1H), 6.85 (d, J = 7.2 Hz, 1H), 5.06 (dd, J = 5.4, 12.8 Hz, 1H), 4.48-4.36 (m, 2H), 4.00 (d, J = 6.8 Hz, 2H), 3.78 (s, 1H), 3.64 (dd, J = 4.4, 10.8 Hz, 1H), 3.50 (t, J = 6.4 |
| Example 427 | | Hz, 2H), 3.43 (d, J = 11.6 Hz, 2H), |
| | | 2.96 2.83 (m, 1H), 2.60 (d, J = 2.4 |
| | | Hz, 1H), 2.22-2.10 (m, 4H), 2.04- |
| | | 1.86 (m, 5H), 1.73-1.59 (m, 2H), |
| | | 1.54-1.43 (m, 1H), 0.70 (d, J = 7.6 |
| | | Hz, 2H), 0.44 (d, J = 4.8 Hz, 2H) |
| 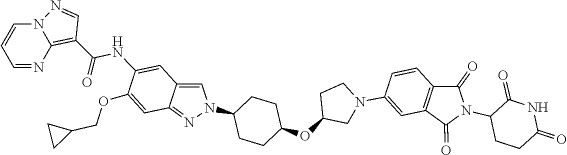 [SMILES: O=C1CCC(N2C(=O)c3ccc(N4CC[C@H](O[C@H]5CC[C@@H](n6cc7cc(NC(=O)c8cnn9cccnc89)c(OCC8CC8)cc7n6)CC5)C4)cc3C2=O)C(=O)N1] | 772.2 | DMSO-d₆ δ 11.07 (s, 1H), 10.64 (s, 1H), 9.37 (d, J = 6.8 Hz, 1H), 8.86 (d, J = 4.0 Hz, 1H), 8.72 (d, J = 10.2 Hz, 2H), 8.25 (s, 1H), 7.65 (d, J = 8.4 Hz, 1H), 7.42-7.28 (m, 1H), 7.02 (s, 1H), 6.94 (s, 1H), 6.84 (d, J = 8.0 Hz, 1H), 5.06 (dd, J = 5.2, 12.8 Hz, 1H), 4.51-4.33 (m, 2H), 4.01 (d, J = 6.8 Hz, 2H), 3.63 (dd, J = 4.8, 11.2 Hz, 2H), 3.51-3.45 (m, 3H), 2.96-2.83 (m, |
| Example 428 | | 2H), 2.17-2.07 (m, 6H), 2.07- 1.90 (m, |
| | | 4H), 1.52-1.37 (m, 3H),0.77- 0.65 |
| | | (m, 2H), 0.51-0.40 (m, 2H) |

### Preparation of Example 429: N-[6-(cyclopropylmethoxy)-2-[(1r,4r)-4-({4-[2-(2,6-dioxopiperidin-3-yl)-1,3-dioxo-2,3-dihydro-1H-isoindol-5-yl]piperidin-1-yl}methyl)cyclohexyl]-2H-indazol-5-yl]pyrazolo[1,5-a]pyrimidine-3-carboxamide

Step 1: 2-((1r,4r)-4-(hydroxymethyl)cyclohexyl)-5-nitro-2H-indazol-6-ol

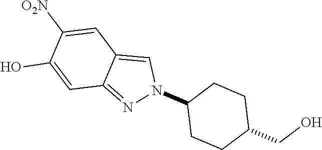 [SMILES: O=[N+]([O-])c1cc2cn([C@H]3CC[C@H](CO)CC3)nc2cc1O]

To a solution of 2-azido-4-hydroxy-5-nitrobenzaldehyde (5 g, 24.0 mmol) in toluene (70 mL) was added sodium sulfate (17.06 g, 120.1 mmol, 12.19 mL) and ((1r,4r)-4-aminocyclohexyl)methanol (3.10 g, 24.0 mmol). The mixture was stirred at 120° C. for 2 h, then concentrated under reduced pressure. The residue was purified by silica gel chromatography (25%-100% ethyl acetate in petroleum ether) to afford 2-((1r,4r)-4-(hydroxymethyl)cyclohexyl)-5-nitro-2H-indazol-6-ol (2.14 g, 31%) as a yellow solid. MS (ESI) m/z: 292.1 [M+H]⁺.

Step 2: ((1r,4r)-4-(6-(cyclopropylmethoxy)-5-nitro-2H-indazol-2-yl)cyclohexyl)methanol

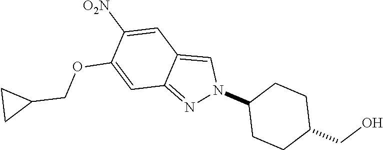 [SMILES: O=[N+]([O-])c1cc2cn([C@H]3CC[C@H](CO)CC3)nc2cc1OCC1CC1]

To a solution of 2-((1r,4r)-4-(hydroxymethyl)cyclohexyl)-5-nitro-2H-indazol-6-ol (2.04 g, 7.0 mmol) in N,N-dimethylformamide (20 mL) was added potassium carbonate (2.90 g, 21.0 mmol) and (bromomethyl)cyclopropane (1.89 g, 14.0 mmol, 1.34 mL). The mixture was stirred at 60° C. for 12 h. The mixture was poured into water (150 mL). The aqueous phase was extracted with ethyl acetate (2×150 mL). The combined organic phase was washed with brine (2×100 mL), dried over anhydrous sodium sulfate, filtered, and concentrated in vacuum. The residue was purified by silica gel chromatography (30%-70% ethyl acetate in petroleum ether) to afford ((1r,4r)-4-(6-(cyclopropylmethoxy)-5-nitro-2H-indazol-2-yl)cyclohexyl)methanol (1.75 g, 72%) as a yellow solid. MS (ESI) m/z: 346.2 [M+H]⁺.

Step 3: ((1r,4r)-4-(5-amino-6-(cyclopropylmethoxy)-2H-indazol-2-yl)cyclohexyl)methanol

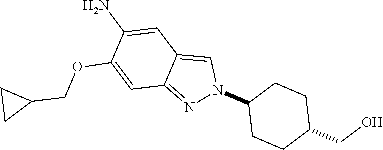 [SMILES: Nc1cc2cn([C@H]3CC[C@H](CO)CC3)nc2cc1OCC1CC1]

To a solution of ((1r,4r)-4-(6-(cyclopropylmethoxy)-5-nitro-2H-indazol-2-yl)cyclohexyl)methanol (1.75 g, 5.1 mmol) in trifluoroethanol (20 mL) was added 10% palladium on carbon (300 mg) under nitrogen atmosphere. The suspension was degassed and purged with hydrogen for 3 times. The mixture was stirred under hydrogen (15 Psi) at 25° C. for 4 h. The mixture was filtered and concentrated under reduced pressure to afford ((1r,4r)-4-(5-amino-6-(cyclopropylmethoxy)-2H-indazol-2-yl)cyclohexyl)methanol (1.55 g, 97%) as a brown solid. MS (ESI) m/z: 316.2 [M+H]⁺.

Step 4: N-(6-(cyclopropylmethoxy)-2-((1r,4r)-4-(hydroxymethyl)cyclohexyl)-2H-indazol-5-yl)pyrazolo[1,5-a]pyrimidine-3-carboxamide

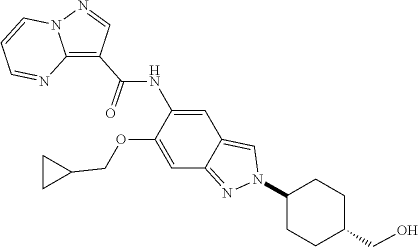 [SMILES: O=C(Nc1cc2cn([C@H]3CC[C@H](CO)CC3)nc2cc1OCC1CC1)c1cnn2cccnc12]

To a solution of ((1r,4r)-4-(5-amino-6-(cyclopropylmethoxy)-2H-indazol-2-yl)cyclohexyl)methanol (1.35 g, 4.3 mmol) and pyrazolo[1,5-a]pyrimidine-3-carboxylic acid (768.06 mg, 4.7 mmol) in pyridine (30 mL) was added 1-(3-dimethylaminopropyl)-3-ethylcarbodiimide hydrochloride (2.46 g, 12.8 mmol). The mixture was stirred at 25° C. for 0.5 h, then 50° C. for 2 h. The mixture was concentrated under reduced pressure. The residue was purified by silica gel chromatography (0%-10% methanol in ethyl acetate) to afford N-(6-(cyclopropylmethoxy)-2-((1r,4r)-4-(hydroxymethyl)cyclohexyl)-2H-indazol-5-yl)pyrazolo[1,5-a]pyrimidine-3-carboxamide (1.4 g, 71%) as a yellow solid. MS (ESI) m/z: 461.3 [M+H]⁺.

Step 5: N-(6-(cyclopropylmethoxy)-2-((1r,4r)-4-formylcyclohexyl)-2H-indazol-5-yl)pyrazolo[1,5-a]pyrimidine-3-carboxamide

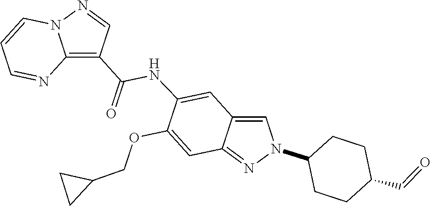 [SMILES: O=C[C@H]1CC[C@H](n2cc3cc(NC(=O)c4cnn5cccnc45)c(OCC4CC4)cc3n2)CC1]

To a solution of N-(6-(cyclopropylmethoxy)-2-((1r,4r)-4-(hydroxymethyl)cyclohexyl)-2H-indazol-5-yl)pyrazolo[1,5-a]pyrimidine-3-carboxamide (200 mg, 434.3 umol) in dichloromethane (15 mL) was added 1,1,1-triacetoxy-1,1-dihydro-1,2-benziodoxol-3-(1H)-one (552.59 mg, 1.3 mmol, 403.35 uL). The mixture was stirred at 25° C. for 2 h, then filtered and concentrated. The residue was purified by silica gel chromatography (0%-5% methanol in dichloromethane) to afford N-(6-(cyclopropylmethoxy)-2-((1r,4r)-4-formylcyclohexyl)-2H-indazol-5-yl)pyrazolo[1,5-a]pyrimidine-3-carboxamide (190 mg, 95%) as a yellow solid. MS (ESI) m/z: 459.2 [M+H]⁺.

Step 6: N-(6-(cyclopropylmethoxy)-2-((1r,4r)-4-((4-(2-(2,6-dioxopiperidin-3-yl)-1,3-dioxoisoindolin-5-yl)piperidin-1-yl)methyl)cyclohexyl)-2H-indazol-5-yl)pyrazolo[1,5-a]pyrimidine-3-carboxamide

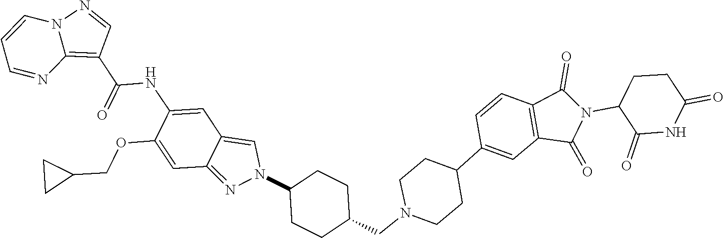 [SMILES: O=C1CCC(N2C(=O)c3ccc(C4CCN(C[C@H]5CC[C@H](n6cc7cc(NC(=O)c8cnn9cccnc89)c(OCC8CC8)cc7n6)CC5)CC4)cc3C2=O)C(=O)N1]

To a solution of N-(6-(cyclopropylmethoxy)-2-((1r,4r)-4-formylcyclohexyl)-2H-indazol-5-yl)pyrazolo[1,5-a]pyrimidine-3-carboxamide (114.17 mg, 249.0 umol) and 2-(2,6-dioxopiperidin-3-yl)-5-(piperidin-4-yl)isoindoline-1,3-dione (85 mg, 249.0 umol) in tetrahydrofuran (4 mL) and N,N-dimethylformamide (1 mL) was added acetic acid (59.81 mg, 996.0 umol, 56.96 uL) at 0° C. The mixture was stirred at 0° C. for 1 h. Then sodium triacetoxyborohydride (263.87 mg, 1.2 mmol) was added and the mixture was stirred at 0° C. for another 1 h. The mixture was poured into saturated sodium bicarbonate aqueous solution (50 mL). The aqueous phase was extracted with dichloromethane (2×50 mL). The combined organic phase was washed with brine (2×40 mL), dried over anhydrous sodium sulfate, filtered, and concentrated in vacuum. The residue was purified by prep-Thin-layer chromatography (dichloromethane:methanol=10:1) then further purified by semi-preparative reverse phase HPLC (column: Unisil 3-100 C18 Ultra 150*50 mm*3 um; mobile phase: [water (formic acid)-acetonitrile]; B %₀: 150%-45%, 10 min) to afford N-(6-(cyclopropylmethoxy)-2-((1r,4r)-4-((4-(2-(2,6-dioxopiperidin-3-yl)-1,3-dioxoisoindolin-5-yl)piperidin-1-yl)methyl)cyclohexyl)-2H-indazol-5-yl)pyrazolo[1,5-a]pyrimidine-3-carboxamide formate (78.9 mg, 370%) as a yellow solid. MS (ESI) m/z: 784.3 [M+H]⁺; ¹H NMR (400 MHz, DMSO-d₆) δ 11.14 (s, 1H), 10.65 (s, 1H), 9.39 (dd, J=1.6, 7.2 Hz, 1H), 8.87 (dd, J=1.6, 4.4 Hz, 1H), 8.73 (d, J=10.4 Hz, 2H), 8.27 (s, 1H), 8.25 (s, 1H), 7.87-7.77 (m, 3H), 7.35-7.30 (m, 1H), 7.04 (s, 1H), 5.14 (dd, J=5.2, 12.8 Hz, 1H), 4.40-4.31 (m, 1H), 4.02 (d, J=6.8 Hz, 2H), 2.99 (br d, J=11.2 Hz, 2H), 2.95-2.85 (m, 1H), 2.81-2.73 (m, 1H), 2.64-2.58 (N, 1H), 2.57-2.54 (O, 1H), 2.20 (br d, J=6.8 Hz, 2H), 2.15 (br d, J=10.8 Hz, 2H), 2.08-1.94 (N, 6H), 1.92-1.87 (m, 1H), 1.84-1.71 (m, 4H), 1.69-1.62 (m, 1H), 1.54-1.46 (m, 1H), 1.19-1.06 (m, 2H), 0.74-0.68 (m, 2H), 0.49-0.43 (m, 2H).

TABLE 18
The following compounds may be prepared in an analogous fashion as described for the
synthesis of Example 429.

| | | ¹H NMR All |
|---|---|---|
| | | spectra acquired |
| | | at 400 MHz |
| | | unless |
| Example | MS | otherwise indicated |
| Example 429 | See Above | |
| 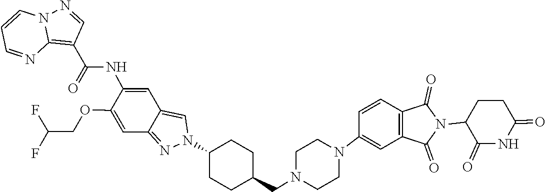 [SMILES: O=C1CCC(N2C(=O)c3ccc(N4CCN(C[C@H]5CC[C@H](n6cc7cc(NC(=O)c8cnn9cccnc89)c(OCC(F)F)cc7n6)CC5)CC4)cc3C2=O)C(=O)N1] | 795.5 | DMSO-d₆ δ 10.98-11.15 (m, 1H) 10.59 (s, 1 H) 9.38 (dd, J =7.20, 1.60 Hz, 1 H ) 8.86 (dd, J = 4.40, 1.60 Hz, 1 H) 8.77 (s, 1 H) 8.71 (s, 1 H) 8.46 (s, 1 H) 8.32 (s, 1 H) 7.68 (d, J = 8.40 Hz, 1 H) 7.34-7.38 (m, 2 H) 7.24-7.28 (m, 6.46-6.80 (m, 1 H) 5.07 (dd, J = 12.80,5.20 Hz, 1 H) 4.49-4.57 (m, 2 H) 4.32-4.43 (m, 1 H) 3.45 (br s, 9 H) 2.81-2.9 (m, 2 H) 2.60 (br d, J = 2.40 Hz, 2 H) 2.55 (br s, 5 H) 2.22 (br d, J = 6.80 Hz, |
| Example 430 | | 2 H) 2.15 (br d, J = 12.40 Hz, 2 H) |
| | | 1.85-2.08 (m, 7 H) 1.63-1.72 (m, 1 |
| | | H) 1.07-1.26 (m, 3 H) |
| 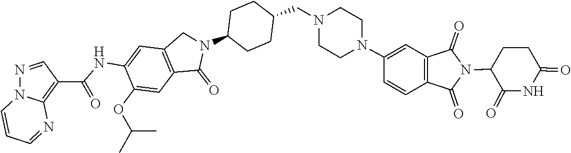 [SMILES: CC(C)Oc1cc2c(cc1NC(=O)c1cnn3cccnc13)CN([C@H]1CC[C@H](CN3CCN(c4ccc5c(c4)C(=O)N(C4CCC(=O)NC4=O)C5=O)CC3)CC1)C2=O] | 788.1 | DMSO-d₆ δ 10.88-11.12 (m, 1 H) 10.75 (s, 1 H) 9.40 (dd, J = 7.20, 1.60 Hz, 1 H) 8.89 (dd, J =4.40, 1.60 Hz, 1 H) 8.73 (s, 2 H) 8.42 (s, 1 H) 7.68 (d, J = 8.40 Hz, 1 H) 7.34-7.38 (m, 1 H) 7.32-7.34 (m, 2 H) 7.26 (dd, J = 8.80, 2.08 Hz, 1 H) 5.07 (dd,J = 12.80, 5.20 Hz, 1 H) 4.89 (quin, J = 6.0 Hz, 1 H) 4.40 (s, 2 H) 3.94-4.05 (m, 1 H) 3.45 (br s, 5 H) |
| Example 431 | | 2.80-2.97 (m, 2 H) 2.60 (br d, J = |
| | | 2.40 Hz, 1 H) 2.55 (br s, 2 H) 2.18 |
| | | (br d, J = 6.80 Hz, 2 H) 1.98-2.07 |
| | | (m, 1H) 1.91 (br d, J = 12.40 Hz, 2 |
| | | H) 1.77 (br d, J = 9.60 Hz, 2 H) |
| | | 1.47-1.68 (m, 4 H) 1.45 (s, 3 H) 1.43 |
| | | (s, 3 H) 0.99-1.12 (m, 2H) |
| 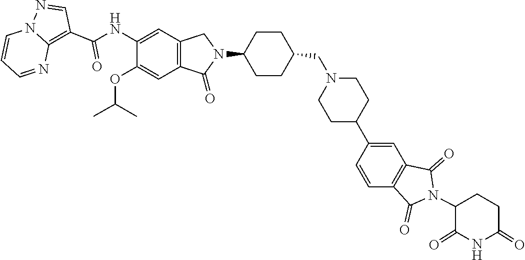 [SMILES: CC(C)Oc1cc2c(cc1NC(=O)c1cnn3cccnc13)CN([C@H]1CC[C@H](CN3CCC(c4ccc5c(c4)C(=O)N(C4CCC(=O)NC4=O)C5=O)CC3)CC1)C2=O] | 787.5 | DMSO-d₆ δ 11.12 (br s, 1 H) 10.75 (s, 1 H) 9.40 (dd, J = 6.80, 1.60 Hz, 1 H) 8.89 (dd, J = 4.40, 1.60 Hz, 1 H) 8.73 (s, 2 H) 8.23 (s, 1 H) 7.82-7.87 (m, 1 H) 7.76-7.81 (m, 2H) 7.31-7.39 (m, 2 H) 5.13 (dd,J = 12.80, 5.20 Hz, 1 H) 4.89 (dt, J = 12.00, 6.11 Hz, 1 H) 4.40 (s, 2 H) 3.90-4.05 (m, 1 H) 2.91-3.01 (m, 3 H) 2.83-2.91 (m, 1 H) 2.72-2.78 (m, 1 H) 2.60-2.64 (m, 1 H) 2.58 (br s, 1 H) 2.17 (br d, J = 6.80 Hz, 2 H) 1.96-2.10 (m, 4 H) 1.88-1.94 (m, 2H) 1.73-1.80 (m, 5 H) 1.56-1.64 (m, 2H) 1.45 (s, 3 H) 1.43 (s, 3 H) 0.96-1.11 (m, 2H) |
| Example 432 | | |
| 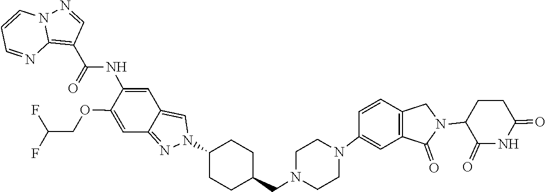 [SMILES: O=C1CCC(N2Cc3ccc(N4CCN(C[C@H]5CC[C@H](n6cc7cc(NC(=O)c8cnn9cccnc89)c(OCC(F)F)cc7n6)CC5)CC4)cc3C2=O)C(=O)N1] | 781.4 | DMSO-d₆ δ 10.97 (s, 1 H) 10.59 (s, 1 H) 9.37 (d, J = 7.20 Hz, 1 H) 8.85 (d, J = 3.60 Hz, 1 H) 8.76 (s, 1 H) 8.71 (s, 1 H) 8.31 (s,1 H) 7.43 (d, J = 8.40 Hz, 1 H) 7.35 (dd, J = 6.80, 4.40 Hz, 1 H) 7.26 (br d, J = 8.80 Hz, 1 H) 7.21 (s, 1 H) 7.17 (s, 1H) 6.46-6.81 (m, 1 H) 5.09 (dd, J = 13.20, 5.20 Hz, 1 H) 4.52 (td, J = 13.60, 3.20 Hz, 2 H) 4.29-4.42 (m, 2 H) 4.16- 4.24 (m, 1 H) 3.90 (s, 1H) 3.21 (br s, 4 H) 2.83-2.94 (m, 1 H) |
| Example 433 | | 2.59 (br d, J = 18.00 Hz, 2 H) 2.53 |
| | | (br s, 2 H) 2.38 (br dd, J = 13.20, |
| | | 4.40 Hz, 1 H) 2.11-2.23 (m, 4 H) |
| | | 1.87-2.02 (m, 5 H) 1.59-1.74 (m, 1 |
| | | H) 1.13 (br d, J = 12.40 Hz, 2 H) |

### Biological Activity of Exemplary Compounds

### IRAK-4 ELISA Protocol

Reagents: Human IRAK-4 Matched Antibody Pair Kit (Abcam ab218182) and ELISA Accessory Pack (Abcam ab210905).

Cell Treatment and Lysis:

1. OCI-Ly10: 200,000 cells are seeded at 1×10⁶/ml in 96-well U-bottom plates 200 ul per well, treated with compound the same day. Incubate overnight.
2. PBMC: 800,000 cells are seeded at 2×10⁶/ml in 96-well U-bottom deep-well plates, 400 ul per well, treated with compound the same day. Incubate overnight.

Cell lysate is collected and analyzed by ELISA for IRAK-4 levels.

TABLE 19
PBMC Degradation Data.

| | PBMC | PBMC | PBMC |
|---|---|---|---|
| Example | DC₉₀ | DC₅₀ | Dₘₐₓ |
| 1 | 1 | A | A |
| 2 | 2 | A | A |
| 3 | 3 | D | B |
| 4 | 4 | A | A |
| 5 | 5 | B | B |
| 6 | 6 | A | A |
| 9 | A | B | B |
| 11 | C | B | A |
| 12 | A | A | A |
| 13 | C | A | A |
| 14 | A | A | A |
| 15 | A | A | B |
| 16 | A | A | A |
| 17 | A | A | B |
| 18 | A | A | A |
| 19 | A | B | B |
| 20 | A | A | A |
| 21 | A | A | A |
| 22 | A | A | B |
| 23 | A | A | A |
| 25 | A | B | A |
| 27 | A | A | A |
| 28 | A | A | A |
| 29 | A | B | B |
| 30 | A | A | B |
| 31 | B | B | A |
| 32 | C | B | C |
| 36 | B | B | D |
| 38 | A | B | A |
| 39 | C | C | A |
| 40 | D | C | B |
| 41 | A | A | B |
| 42 | B | B | A |
| 43 | B | B | A |
| 44 | D | D | B |
| 45 | A | B | A |
| 46 | A | B | A |
| 47 | A | B | A |
| 48 | A | A | A |
| 49 | A | A | A |
| 50 | A | A | A |
| 51 | A | A | A |
| 52 | A | B | A |
| 53 | B | B | A |
| 54 | A | B | A |
| 55 | A | A | A |
| 56 | A | B | A |
| 57 | A | A | A |
| 58 | B | B | A |
| 59 | B | B | A |
| 60 | D | B | A |
| 61 | A | A | A |
| 62 | B | B | A |
| 64 | A | A | A |
| 65 | A | B | A |
| 66 | A | B | A |
| 67 | C | B | A |
| 68 | A | A | A |
| 69 | A | B | A |
| 70 | A | A | A |
| 71 | B | B | A |
| 72 | A | A | B |
| 73 | D | C | C |
| 74 | D | C | B |
| 75 | B | B | A |
| 76 | B | B | A |
| 77 | B | B | A |
| 78 | A | A | A |
| 79 | A | A | A |
| 80 | A | B | A |
| 81 | A | A | A |
| 82 | A | A | A |
| 83 | A | B | A |
| 84 | A | A | A |
| 85 | C | B | B |
| 86 | B | B | A |
| 87 | D | C | B |
| 88 | A | A | A |
| 89 | B | B | B |
| 90 | A | A | A |
| 91 | B | B | A |
| 92 | B | B | A |
| 93 | A | A | A |
| 94 | A | B | A |
| 95 | A | A | A |
| 96 | B | B | A |
| 97 | A | A | B |
| 98 | D | C | C |
| 99 | D | C | B |
| 100 | B | B | A |
| 101 | B | B | C |
| 102 | C | B | C |
| 103 | A | A | B |
| 104 | A | A | C |
| 105 | D | D | nt |
| 106 | A | B | A |
| 107 | A | B | C |
| 108 | B | B | A |
| 109 | A | A | A |
| 110 | B | B | A |
| 111 | A | A | B |
| 112 | A | B | A |
| 114 | A | A | A |
| 115 | A | A | A |
| 116 | B | B | A |
| 117 | B | B | A |
| 119 | A | A | A |
| 122 | B | B | D |
| 124 | A | A | A |
| 126 | B | B | D |
| 131 | A | A | A |
| 132 | A | A | A |
| 133 | A | A | A |
| 134 | A | A | A |
| 136 | A | A | A |
| 137 | A | A | A |
| 138 | A | A | A |
| 139 | A | A | B |
| 141 | B | B | B |
| 142 | A | A | B |
| 143 | C | B | A |
| 147 | B | B | A |
| 148 | A | A | B |
| 149 | A | A | B |
| 150 | C | B | B |
| 151 | D | C | C |
| 152 | D | D | D |
| 153 | A | A | B |
| 154 | A | A | B |
| 155 | A | A | A |
| 156 | A | A | A |
| 157 | A | A | A |
| 158 | A | A | A |
| 159 | A | B | A |
| 161 | B | B | A |
| 165 | A | B | A |
| 166 | C | C | A |
| 167 | A | A | A |
| 169 | 122 | A | A |
| 170 | A | A | A |
| 173 | A | A | A |
| 174 | B | B | A |
| 175 | B | B | A |
| 176 | A | A | A |
| 177 | A | A | A |
| 182 | A | A | A |
| 183 | B | B | A |
| 184 | C | B | B |
| 185 | A | A | A |
| 186 | A | B | A |
| 187 | A | A | A |
| 188 | D | C | A |
| 189 | A | A | A |
| 190 | D | D | A |
| 191 | D | C | B |
| 192 | A | B | B |
| 193 | A | A | A |
| 194 | C | B | A |
| 195 | B | B | A |
| 196 | B | B | B |
| 197 | A | A | B |
| 198 | B | C | A |
| 199 | B | B | A |
| 200 | A | B | A |
| 202 | A | A | A |
| 207 | B | B | A |
| 212 | A | A | A |
| 214 | A | A | C |
| 215 | A | B | C |
| 216 | A | A | B |
| 217 | A | A | B |
| 218 | D | C | B |
| 219 | C | C | B |
| 220 | C | B | B |
| 221 | B | B | D |
| 222 | D | D | C |
| 223 | C | B | C |
| 224 | B | B | B |
| 225 | B | B | B |
| 227 | A | B | B |
| 228 | C | B | B |
| 229 | B | C | B |
| 230 | D | B | A |
| 231 | A | B | B |
| 232 | C | B | B |
| 233 | C | C | B |
| 234 | A | A | B |
| 235 | A | A | A |
| 236 | A | A | C |
| 237 | D | B | C |
| 238 | B | B | C |
| 239 | C | C | D |
| 240 | A | A | B |
| 241 | B | B | C |
| 242 | A | B | B |
| 243 | D | C | B |
| 244 | B | B | B |
| 245 | A | B | C |
| 246 | B | C | D |
| 247 | B | B | A |
| 248 | A | B | D |
| 249 | A | B | A |
| 250 | B | B | B |
| 251 | A | B | B |
| 253 | B | B | A |
| 254 | A | B | A |
| 255 | D | D | A |
| 256 | A | A | A |
| 257 | A | B | A |
| 258 | A | A | B |
| 261 | A | B | A |
| 262 | A | B | C |
| 263 | A | D | A |
| 264 | B | B | C |
| 265 | A | B | A |
| 268 | A | A | A |
| 269 | A | B | B |
| 270 | D | D | D |
| 271 | D | C | B |
| 273 | A | A | A |
| 274 | A | A | B |
| 275 | A | A | A |
| 276 | A | A | B |
| 277 | A | A | A |
| 278 | A | A | B |
| 279 | A | A | A |
| 282 | A | A | A |
| 283 | A | A | A |
| 284 | A | A | A |
| 285 | A | B | B |
| 286 | B | B | A |
| 287 | B | B | A |
| 288 | A | A | A |
| 291 | B | B | A |
| 292 | A | A | A |
| 293 | B | B | A |
| 296 | A | A | A |
| 297 | B | B | A |
| 298 | D | D | A |
| 303 | C | C | B |
| 305 | C | C | B |
| 306 | D | D | C |
| 307 | D | D | nt |
| 309 | B | B | A |
| 310 | A | B | A |
| 311 | D | C | B |
| 312 | A | B | A |
| 313 | A | A | A |
| 314 | A | A | B |
| 315 | D | C | D |
| 316 | A | A | A |
| 317 | A | A | A |
| 318 | A | A | A |
| 319 | A | B | B |
| 320 | D | D | B |
| 321 | A | B | A |
| 322 | A | B | A |
| 323 | A | A | A |
| 324 | A | A | A |
| 325 | C | B | A |
| 326 | B | B | A |
| 327 | D | D | B |
| 328 | A | A | A |
| 329 | A | A | A |
| 330 | A | A | A |
| 331 | B | B | A |
| 332 | D | D | A |
| 334 | A | A | A |
| 335 | A | A | A |
| 336 | A | A | A |
| 337 | A | B | A |
| 338 | A | A | A |
| 339 | A | A | A |
| 341 | A | A | A |
| 342 | A | A | A |
| 343 | A | A | A |
| 344 | A | A | A |
| 345 | A | A | A |
| 346 | B | B | B |
| 347 | B | B | B |
| 348 | A | A | A |
| 349 | A | A | B |
| 350 | B | B | C |
| 351 | A | A | B |
| 352 | B | B | B |
| 353 | B | B | B |
| 354 | D | A | C |
| 355 | D | B | B |
| 356 | A | B | A |
| 357 | A | A | C |
| 358 | A | A | D |
| 359 | A | A | B |
| 360 | B | B | A |
| 361 | A | A | A |
| 362 | A | A | A |
| 363 | A | A | A |
| 364 | A | B | A |
| 365 | A | B | A |
| 366 | A | B | A |
| 367 | D | D | B |
| 368 | A | B | A |
| 369 | A | B | A |
| 370 | A | B | A. |
| 371 | B | A | A |
| 371′ | B | B | A |
| 372 | A | A | A |
| 373 | A | A | A |
| 374 | B | A | A |
| 375 | A | B | A |
| 376 | A | B | A |
| 377 | B | B | A |
| 378 | A | A | A |
| 379 | A | A | A |
| 381 | A | A | A |
| 382 | A | A | A |
| 383 | A | A | A |
| 384 | A | A | A |
| 385 | A | A | A |
| 386 | A | A | A |
| 387 | A | A | A |
| 388 | A | A | A |
| 389 | A | A | B |
| 390 | A | A | A |
| 397 | A | A | A |
| 398 | A | A | A |
| 399 | B | B | A |
| 400 | D | C | B |
| 401 | B | B | A |
| 402 | A | A | A |
| 403 | C | B | A |
| 404 | D | D | B |
| 407 | A | A | A |
| 408 | A | A | A |
| 409 | B | B | A |
| 410 | D | C | B |
| 411 | D | C | B |
| 412 | A | B | B |
| 413 | B | B | B |
| 414 | B | B | B |
| 415 | A | B | B |
| 416 | A | A | B |
| 417 | A | A | A |
| 418 | A | A | B |
| 419 | A | B | C |
| 420 | A | A | A |
| 421 | A | A | B |
| 422 | C | B | C |
| 423 | B | B | A |
| 424 | B | B | B |
| 425 | D | D | nt |
| 426 | D | D | A |
| 427 | D | D | nt |
| 428 | B | B | A |
| 429 | A | A | A |
| 430 | A | B | A |
| 431 | A | B | A |
| 432 | A | B | A |
| 433 | A | A | A |

PBMC DC₉₀: A, B, C, or D. According to the code, A represents a DC₉₀ value <10.0 nM; B represents a DC₉₀ value ≥10.0 nM and <25.0 nM; C represents a DC₉₀ value ≥25.0 nM and <50 nM; D represents a DC₉₀ value ≥50 nM.
PBMC DC₅₀: A, B, C, or D. According to the code, A represents a DC₅₀ value <1.0 nM; B represents a DC₅₀ value ≥1.0 nM and <5.0 nM; C represents a DC₅₀ value ≥5.0 nM and <10 nM; D represents a DC₅₀ value ≥10 nM.
PBMC Dₘₐₓ: A, B, C, or D. According to the code, A represents a Dₘₐₓ value ≥90; B represents a Dₘₐₓ value ≥80 and <90; C represents a Dₘₐₓ value ≥70 and <80; D represents a Dₘₐₓ value <70.
nt = not measured

TABLE 20
OCI-Ly10 and OCI-Ly1 Degradation Data.

| | OCI-LY10 | OCI-LY10 |
|---|---|---|
| Example | DC₅₀ | Dₘₐₓ |
| 1 | A | A |
| 4 | A | A |
| 5 | A | A |
| 7 | B | B |
| 8 | A | A |
| 9 | A | A |
| 10 | D | nt |
| 12 | A | A |
| 23 | A | B |
| 26 | D | nt |
| 29 | A | C |
| 30 | A | A |
| 31 | A | B |
| 32 | A | D |
| 33 | B | B |
| 34 | D | nt |
| 35 | B | A |
| 36 | A | D |
| 37 | D | nt |
| 38 | A | A |
| 57 | B | A |
| 58 | B | A |
| 59 | A | A |
| 63 | B | A |
| 64 | A | A |
| 69 | A | A |
| 82 | A | A |
| 86 | A | A |
| 87 | C | A |
| 88 | C | B |
| 91 | B | A |
| 93 | B | A |
| 94 | B | C |
| 96 | B | A |
| 97 | A | A |
| 98 | B | A |
| 99 | A | A |
| 118 | A | A |
| 119 | A | B |
| 120 | B | A |
| 121 | B | A |
| 122 | A | D |
| 123 | D | nt |
| 125 | B | C |
| 126 | A | A |
| 127 | B | A |
| 128 | B | A |
| 129 | A | B |
| 130 | C | A |
| 134 | A | A |
| 135 | A | B |
| 136 | A | C |
| 140 | B | B |
| 141 | A | B |
| 142 | A | C |
| 143 | A | A |
| 144 | D | nt |
| 145 | B | A |
| 146 | D | nt |
| 160 | A | A |
| 161 | B | A |
| 162 | A | A |
| 163 | B | A |
| 164 | B | A |
| 165 | B | A |
| 166 | B | A |
| 167 | A | A |
| 168 | B | A |
| 171 | C | C |
| 172 | D | B |
| 173 | A | A |
| 178 | D | B |
| 179 | D | D |
| 180 | B | A |
| 181 | D | B |
| 182 | A | A |
| 184 | A | D |
| 201 | A | A |
| 202 | A | A |
| 203 | A | A |
| 204 | C | D |
| 205 | A | B |
| 206 | A | A |
| 207 | A | A |
| 208 | B | A |
| 209 | D | nt |
| 210 | D | nt |
| 211 | D | nt |
| 212 | A | A |
| 213 | D | D |
| 274 | A | D |
| 278 | A | B |
| 279 | A | C |
| 282 | A | A |
| 285 | A | A |
| 287 | A | A |
| 288 | A | A |
| 289 | B | A |
| 290 | C | D |
| 294 | B | B |
| 295 | B | A |
| 299 | B | A |
| 313 | A | A |
| 333 | D | nt |
| 334 | A | A |
| 335 | A | A |
| 337 | A | A |
| 338 | A | A |
| 339 | A | A |
| 340 | B | A |
| 379 | A | A |
| 380 | D | nt |
| 391 | B | B |
| 392 | C | C |
| 405 | D | C |
| 406 | C | D |

OC1-LY10 DC₅₀: A, B, C, or D. According to the code, A represents a DC₅₀ value <10.0 nM; B represents a DC₅₀ value ≥10.0 nM and <50.0 nM; C represents a DC₅₀ value ≥50.0 nM and <100 nM; D represents a DC₅₀ value ≥100 nM.
OC1-LY10 Dₘₐₓ: A, B, C, or D. According to the code, A represents a Dₘₐₓ value ≥90; B represents a Dₘₐₓ value ≥80 and <90; C represents a Dₘₐₓ value ≥70 and <80; D represents a Dₘₐₓ value <70.
nt = not measured

### IL-1β, LPS, or R848 Stimulated IL6 Inhibition and IL-1β Stimulated IL8 Inhibition Assay

Human PBMC cells were seeded at 2.0×10⁶ cells/mL, treated with compound overnight, and dosed with the EC50 concentration of each stimulant (either IL-1B. LPS, or R848) was determined for each donor. After 24 hrs, media was collected and analyzed by MSD for IL6 and/or IL8 levels.

TABLE 21
Inhibition of IL6 and IL8 in PBMCs after stimulation with IL-1β

| | IL1β | IL1β | IL1β | IL1β |
|---|---|---|---|---|
| | stim IL6 | stim IL6 | stim IL8 | stim IL8 |
| | inhibition | inhibition | inhibition | inhibition |
| Example | IC₅₀ | Yₘₐₓ | IC₅₀ | Yₘₐₓ |
| 1 | A | A | B | C |
| 4 | A | A | B | B |
| 5 | B | B | C | B |
| 6 | A | A | B | B |
| 9 | A | A | B | C |
| 12 | B | B | B | D |
| 13 | B | A | D | A |
| 22 | A | A | B | C |
| 23 | A | A | B | B |
| 29 | B | D | C | C |
| 30 | B | A | D | n/a |
| 31 | D | A | nt | nt |
| 32 | B | A | D | A |
| 36 | B | C | D | n/a |
| 38 | A | A | D | A |
| 59 | B | A | C | C |
| 64 | B | B | C | D |
| 66 | B | A | C | C |
| 67 | B | A | D | A |
| 69 | B | A | B | B |
| 86 | A | A | B | C |
| 96 | C | A | D | D |
| 97 | A | A | B | A |
| 99 | B | A | C | B |
| 119 | A | A | B | B |
| 122 | B | A | C | B |
| 126 | A | A | A | D |
| 134 | A | A | B | C |
| 136 | A | A | C | C |
| 141 | A | A | B | B |
| 142 | A | A | B | D |
| 143 | A | A | A | C |
| 161 | B | B | D | n/a |
| 165 | A | B | B | C |
| 167 | B | A | D | B |
| 173 | B | A | B | D |
| 182 | A | B | A | D |
| 184 | B | B | B | D |
| 202 | B | B | D | n/a |
| 207 | B | A | C | D |
| 212 | B | B | C | D |
| 274 | B | A | C | C |
| 278 | B | A | B | A |
| 279 | B | A | D | B |
| 282 | A | A | B | D |
| 283 | A | A | B | D |
| 285 | A | B | B | D |
| 287 | C | A | D | D |
| 288 | B | A | C | C |
| 313 | A | A | A | D |
| 314 | A | A | B | C |
| 334 | A | A | B | D |
| 335 | A | A | D | n/a |
| 336 | A | A | B | C |
| 337 | A | A | A | D |
| 338 | A | A | B | D |
| 339 | A | A | B | C |
| 379 | A | B | B | C |
| 398 | A | A | D | n/a |
| 400 | D | A | D | n/a |

IL-1β stimulated IL6 inhibition/IL-1β stimulated IL8 inhibition IC₅₀: A, B, C, or D. According to the code, A represents an IC₅₀ value <10.0 nM; B represents an IC₅₀ value ≥10.0 nM and <50.0 nM; C represents an IC₅₀ value ≥50.0 nM and <100 nM; D represents an IC₅₀ value ≥100 nM.
IL-1β stimulated IL6 inhibition/IL-1β stimulated IL8 inhibitionYₘₐₓ: A, B, C, or D. According to the code, A represents a Yₘₐₓ value ≥90; B represents a Yₘₐₓ value ≥80 and <90; C represents a Yₘₐₓ value ≥70 and <80; D represents a Yₘₐₓ value <70.
nt = not measured
n/a = compounds did not show a response in the cytokine inhibition assay.

TABLE 22

| | LPS | LPS | R848 | R848 |
|---|---|---|---|---|
| | Stim IL6 | stim IL6 | stim IL6 | stim IL6 |
| | inhibition | inhibition | inhibition | inhibition |
| Example | Dₘₐₓ | IC₅₀ | Dₘₐₓ | IC₅₀ |
| 1 | B | A | A | A |
| 19 | B | B | A | B |
| 23 | C | B | A | B |
| 41 | C | B | A | A |
| 42 | D | C | D | A |
| 43 | D | A | B | B |
| 44 | C | C | A | B |
| 45 | C | B | A | A |
| 46 | C | B | A | B |
| 47 | B | B | B | B |
| 48 | C | B | A | B |
| 49 | A | A | A | A |
| 50 | D | B | A | B |
| 51 | D | C | A | B |
| 52 | B | B | B | B |
| 53 | D | C | C | B |
| 54 | D | A | B | B |
| 55 | D | A | A | B |
| 56 | C | A | A | B |
| 57 | C | B | A | C |
| 82 | C | B | A | B |
| 88 | C | B | nt | nt |
| 97 | A | A | A | B |
| 105 | C | D | B | C |
| 106 | B | D | A | B |
| 107 | A | B | A | C |
| 108 | B | B | A | B |
| 109 | C | B | A | B |
| 110 | B | B | A | B |
| 111 | A | B | A | B |
| 112 | A | B | A | B |
| 114 | A | B | A | B |
| 115 | C | A | A | A |
| 116 | B | A | A | A |
| 117 | C | B | B | B |
| 128 | C | B | B | B |
| 149 | C | B | A | A |
| 150 | B | C | A | B |
| 153 | A | A | B | A |
| 154 | B | A | A | A |
| 155 | B | A | A | A |
| 156 | C | A | A | A |
| 157 | C | B | A | B |
| 158 | B | A | A | A |
| 159 | C | B | A | B |
| 161 | C | B | A | B |
| 165 | C | B | A | B |
| 218 | C | B | A | A |
| 222 | C | C | A | C |
| 224 | B | B | A | B |
| 231 | B | B | A | B |
| 240 | B | C | A | B |
| 241 | nt | D | B | B |
| 243 | D | B | A | B |
| 245 | B | B | A | A |
| 246 | C | A | A | A |
| 248 | D | B | A | B |
| 249 | D | D | B | B |
| 250 | D | D | B | B |
| 251 | D | C | B | B |
| 253 | D | B | A | B |
| 254 | D | C | A | B |
| 255 | C | C | A | C |
| 256 | D | B | B | B |
| 257 | C | B | A | B |
| 258 | C | A | A | B |
| 261 | B | B | B | C |
| 262 | D | C | A | B |
| 263 | D | A | A | B |
| 265 | A | B | nt | nt |
| 268 | C | B | A | B |
| 269 | B | B | A | B |
| 303 | C | B | A | B |
| 305 | D | B | D | C |
| 306 | C | D | A | D |
| 307 | nt | D | B | D |
| 309 | D | B | B | B |
| 310 | C | D | A | C |
| 311 | C | D | A | B |
| 312 | A | B | A | A |
| 313 | B | B | A | A |
| 346 | B | A | A | B |
| 353 | C | B | A | B |
| 357 | A | A | A | A |
| 358 | C | A | A | A |
| 359 | B | A | A | A |
| 360 | B | A | A | A |
| 361 | B | A | A | A |
| 362 | C | B | A | A |
| 363 | C | B | A | B |
| 364 | D | C | A | A |
| 365 | D | A | A | B |
| 366 | B | A | A | A |
| 367 | B | B | A | B |
| 368 | C | B | A | B |
| 369 | D | B | A | B |
| 370 | A | A | A | A |
| 371 | C | B | A | B |
| 371′ | D | B | A | B |
| 372 | B | A | A | B |
| 373 | C | B | B | B |
| 374 | C | B | A | B |
| 375 | C | A | A | B |
| 376 | C | B | A | B |
| 378 | B | A | A | A |
| 381 | C | A | A | B |
| 382 | C | B | A | B |
| 384 | C | D | A | A |
| 389 | A | D | A | B |
| 402 | D | B | B | B |
| 418 | B | B | A | A |
| 419 | C | A | B | B |
| 423 | B | B | A | B |
| 424 | D | A | B | A |
| 425 | C | C | C | C |
| 426 | D | C | B | C |
| 427 | C | A | D | B |
| 428 | D | B | A | B |
| 429 | C | A | A | B |
| 430 | C | B | A | B |
| 431 | C | B | A | B |
| 432 | C | B | A | B |
| 433 | B | A | A | A |

LPS stimulated IL6 inhibition/R848 stimulated IL6 inhibition IC₅₀: A, B, C, or D. According to the code, A represents an IC₅₀ value <10.0 nM; B represents an IC₅₀ value ≥10.0 nM and <50.0 nM; C represents an IC₅₀ value ≥50.0 nM and <100 nM; D represents an IC₅₀ value ≥100 nM.
LPS stimulated IL6 inhibition/R848 stimulated IL6 inhibition Dₘₐₓ: A, B, C, or D. According to the code, A represents a Yₘₐₓ value ≥90; B represents a Yₘₐₓ value ≥80 and <90; C represents a Yₘₐₓ value ≥70 and <80; D represents a Yₘₐₓ value <70.
nt = not measured
n/a = compounds did not show a response in the cytokine inhibition assay.

PD Study for with in C.B.17 SCID and WT Mice

Cell culture: OCI-Ly1 tumor cells were maintained as a suspension in IMDM medium supplemented with 20% fetal bovine serum under an atmosphere of 5% C02 in air at 37° C. Cell density was maintained below 1×106/mL.

Animals: Female C.B. 17 SCID mice aged 6-8 weeks weighing between 16-18 g were used and maintained according to IACUC protocols.

Tumor Inoculation: Each mouse was inoculated with OCI-Ly1 tumor cells (10×106) in 0.1 mL of RPMI with Matrigel. Treatment was initiated when the tumor sizes reached 400 m3.

A PD Study in C.B.17 SCID Mice with OCI-LY1 Tumor Xenograft was Performed

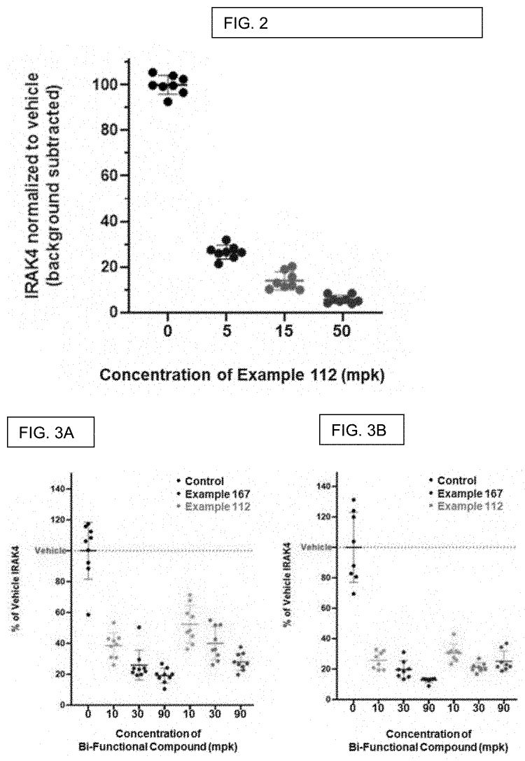

In FIG. 2, animals were dosed orally after tumors were inoculated and the tumor sizes reached 400 m3, with 5, 15, or 50 mg/kg formulated with 10% DMSO, 90% (2% Tween 80 in PEG 400) and sacrificed humanely 24 hours post-dose. Blood was obtained for isolation of plasma for terminal compound exposure. Tumors was removed and divided in 2 portions, one each for terminal compound concentration and for determination of IRAK-4 content. Compound concentrations were determined by LC/MS with calibrated standards. IRAK-4 content in tumor lysate was quantified by ELISA and normalized to total protein concentration per sample. Tumor samples were lysed by homogenization in T-PER and Halt Protease and Phosphatase inhibitors. It was found that IRAK-4 concentration in the tumor samples were lower in the cells treated with the high concentrations of Example 112.

### A PD Study in Wild Type Mice was Performed

In FIGS. 3A and 3B, mice were randomly assigned to each treatment group. Animals were dosed orally with 10, 30, or 90 mg/kg compound formulated in 10% DMSO, 90% (2% Tween 80 in PEG 400) and terminated 24 h later. At termination, plasma, spleen and lung tissue were collected for analysis of compound concentration and IRAK-4 quantification. Spleen and lung were snap frozen. IRAK-4 content in any tissue is assessed with the same protocol described for tumor lysates. It was found that IRAK-4 concentration in the cell samples were much lower in the cells treated with Example 167 (FIG. 3A) and Example 112 (FIG. 3B).

### EMBODIMENTS

The aspects of the present disclosure are further described with reference to the following numbered embodiments:

1. A bifunctional compound having the structure of Formula (I):

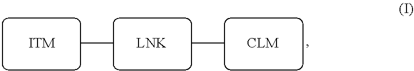

or a pharmaceutically acceptable salt, solvate, enantiomer, stereoisomer, or isotopic derivative thereof,
wherein:
   (a) ITM is an Interleukin-1 Receptor-Associated Kinase 4 (IRAK-4) targeting moiety having the structure of formula ITM-I:

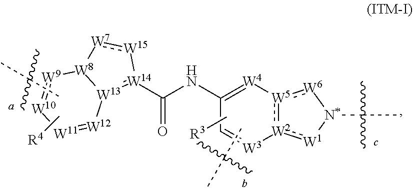

wherein:
[symbol] is a single bond or a double bond;
N* is N or NH
W¹ and W⁶ are each independently C═O, CH, O, N, CH₂, CR¹, or NR¹;
W², W³, W⁴, W₅, W⁷, W⁸, W⁹, W¹⁰, W¹¹, W¹², W¹³, W¹⁴, and W¹⁵ are each independently C, N, C(R¹)ₘ, or NR¹;
R¹ is H, —Cl, —F, —Br, —I, N(R²)₂; linear or branched C₁₋₆ alkyl, C₁₋₆ haloalkyl, 3- to 7-membered heterocycloalkyl, or 5- to 6-membered heteroaryl optionally substituted with one, two, three, four, or five R⁶;
R³ and R⁴ are each independently selected from the group consisting of H, —Cl, —F, —Br, —I, linear or branched C₁₋₆ alkyl optionally substituted with one, two, three, four, or five R⁷, OC₁₋₆ alkyl, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C3-7 cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, and C₁₋₆alkyl-C₃₋₇cycloalkyl optionally substituted with one, two, three, four, or five R⁷;
R^{N} is H, linear or branched C₁₋₆ alkyl, or C₁₋₆ haloalkyl;
each R² is independently selected from the group consisting of H and linear or branched C₁₋₆ alkyl;
each R⁶ is independently selected from the group consisting of OH, OC₁₋₆ alkyl, and heterocycloalkyl;
each R⁷ is independently selected from the group consisting of —Cl, —F, —Br, —I, NH₂, CN, CF₃, linear or branched C₁₋₆ alkyl, OC₁₋₆ alkyl, NH(C₁-C₆ alkyl), N(C₁₋₆ alkyl)(C₁₋₆ alkyl), C₆-C₁₀ aryl, 5- to 6-membered heteroaryl, C₃-C₇ cycloalkyl, and 3- to 7-membered heterocycloalkyl;
m is 1 or 2; and

ITM is connected to LNK through one of

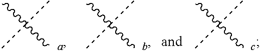

(b) LNK is a chemical linking moiety that covalently couples the ITM to the CLM, having the structure:
[symbol] L [symbol] ₙ
wherein:
each L is independently selected from the group consisting of X, Y, and Z;
   each X is independently selected from the group consisting of

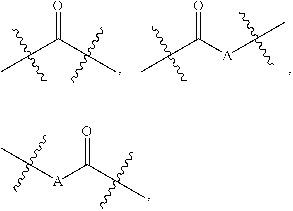

and -A-, wherein each A is independently selected from the group consisting of C(R^{8A})₂, NR⁸, and O;
each R^{8A} is independently selected from the group consisting of H, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, —Cl, —F, —Br, —I, and OH;
each R⁸ is independently selected from the group consisting of H, linear or branched C₁₋₆ alkyl optionally substituted with —Cl, —F, —OH, NH₂, CN, or CF₃, O—C₁₋₆ alkyl, and C₁₋₆ haloalkyl;
each Y is independently selected from the group consisting of

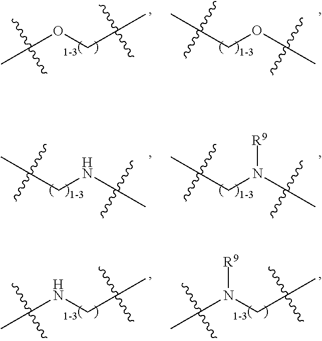

C₁₋₆ alkylene, C₂₋₆ alkenylene, and C₂₋₆ alkynylene;
wherein each Y is independently optionally substituted with one, two, three, four, or five R⁹;
each R⁹ is independently selected from the group consisting of linear or branched C₁₋₆ alkyl optionally substituted with —Cl, —F, —OH, NH₂, CN, or CF₃; O—C₁₋₆ alkyl optionally substituted with —Cl, —F, —OH, NH₂, CN, or CF₃, C₁₋₆ haloalkyl, NH, CN, CF₃, —Cl, —F, —Br, —I, and OH;
each Z is independently selected from the group consisting of monocyclic C₄₋₁₀ cycloalkylene, fused bicyclic C₄₋₁₀ cycloalkylene, bridged bicyclic C₄₋₁₀ cycloalkylene, or spiro-fused bicyclic C₄₋₁₀ cycloalkylene, 4-6 membered heterocycloalkylene, C₆-C₁₀ arylene, and 5-6 membered heteroarylene;
wherein each Z is independently optionally substituted with one, two, three, four, or five R¹⁰;
each R¹⁰ is independently selected from the group consisting of —Cl, —F, —Br, —I, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, and OH; and
n is 1, 2, 3, 4, 5, or 6;
   (c) CLM is a cereblon E3 ubiquitin ligase binding moiety having a structure selected from the group consisting of formulae CLM-I, CLM-II, and CLM-III:

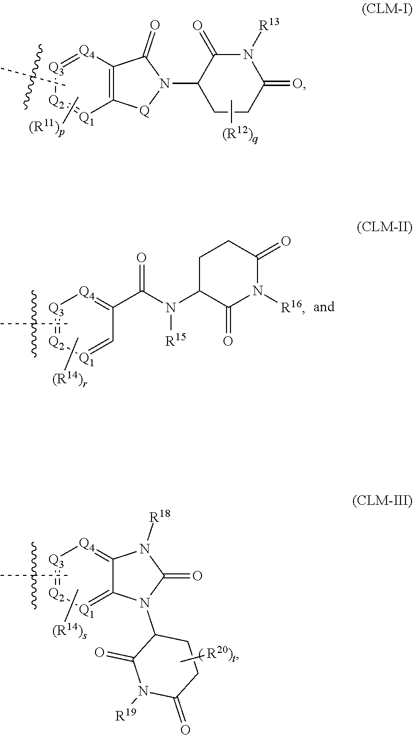

wherein:
Q¹, Q², Q³, and Q⁴ are each independently selected from the group consisting of CH, C, and N;
each R¹¹ is independently selected from the group consisting of —Cl, —F, —Br, —I, CN, CF₃, NH₂, NH—(C1-C₆ alkyl), N(C1-C₆ alkyl)(C1-C₆ alkyl), OH, O—C₁₋₆ alkyl optionally substituted with R²¹, and linear or branched C₁₋₆ alkyl optionally substituted with R²¹;
p is 0, 1, 2, 3, or 4;
Q is selected from the group consisting of CH₂, CH(C₁₋₆ alkyl), C═O, SO₂, NH, and N(C₁₋₆ alkyl);
each R¹² is independently selected from the group consisting of —Cl, —F, —Br, —I, O—C₁₋₆ alkyl optionally substituted with R²¹, NH₂, NH—(C1-C₆ alkyl), N(C1-C₆ alkyl)(C1-C₆ alkyl), OH, CN, CF₃, and linear or branched C1-6 alkyl optionally substituted with R²¹;
q is 0, 1, 2, or 3;
R¹³ is selected from the group consisting of H, OH, linear or branched C₁₋₆ alkyl optionally substituted with R²¹, C(O)C₁₋₆ alkyl, C(O)NH—C₁₋₆ alkyl and C(O)OC₁₋₆ alkyl;
each R¹⁴ is independently selected from the group consisting of —Cl, —F, —Br, —I, NH₂, NH—(C1-C₆ alkyl), N(C1-C₆ alkyl)(C1-C₆ alkyl), OH, CN, CF₃, O—C₁₋₆ alkyl optionally substituted with R²¹, and linear or branched C₁₋₆ alkyl optionally substituted with R²¹;
r is 0, 1, 2, 3, or 4;
R¹⁵ is selected from the group consisting of H and linear or branched C₁₋₆ alkyl optionally substituted with R²¹;
R¹⁶ is selected from the group consisting of H, OH, linear or branched C₁₋₆ alkyl optionally substituted with R²¹, C(O)C₁₋₆ alkyl, C(O)NH—C₁₋₆ alkyl, and C(O)OC₁₋₆ alkyl;
s is 0, 1, 2, or 3;
R¹⁸ is selected from the group consisting of H and linear or branched C₁₋₆ alkyl optionally substituted with R²¹;
R¹⁹ is selected from the group consisting of H, OH, linear or branched C₁₋₆ alkyl optionally substituted with R²¹, C(O)C₁₋₆ alkyl, C(O)NH—C₁₋₆ alkyl, and C(O)OC₁₋₆ alkyl;
each R²⁰ is independently selected from the group consisting of —Cl, —F, —Br, —I, O—C₁₋₆ alkyl optionally substituted with R²¹, NH₂, NH—(C1-C₆ alkyl), N(C1-C₆ alkyl)(C1-C₆ alkyl), OH, CN, CF₃, and linear or branched C₁₋₆ alkyl optionally substituted with R²¹;
t is 0, 1, 2, or 3;
each R²¹ is independently selected from the group consisting of —Cl, —F, —OH, NH₂, CN, and CF₃; and
[symbol] is the point of attachment to LNK. [symbol]
   2. The bifunctional compound of embodiment 1, wherein CLM has a structure CLM-I.
   3. The bifunctional compound of embodiment 2, wherein q is 0 or R³ or methyl.
   4. The bifunctional compound of embodiment 1, wherein CLM has a structure CLM-II.
   5. The bifunctional compound of embodiment 4, wherein R¹⁵ is H or methyl and R¹⁶ is H or methyl.
   6. The bifunctional compound of embodiment 1, wherein CLM has a structure CLM-III.
   7. The bifunctional compound of embodiment 6, wherein t is 0 or R¹⁹ is methyl.
   8. The bifunctional compound of embodiment 1, wherein CLM has the structure selected from the group consisting of:

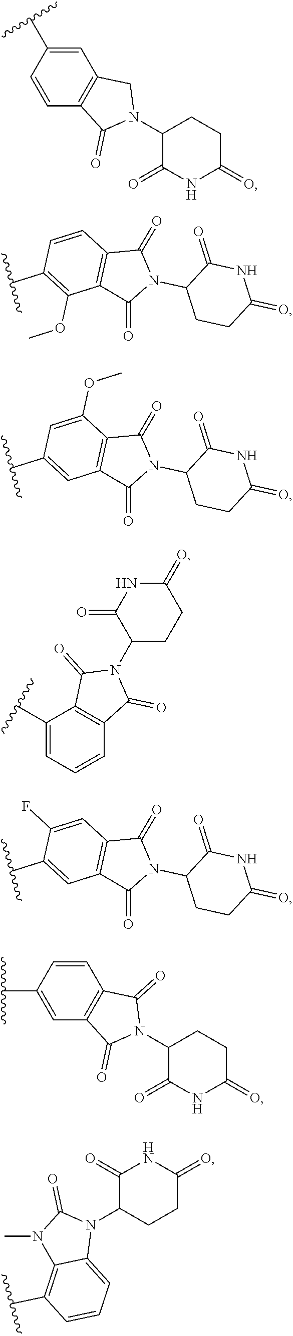

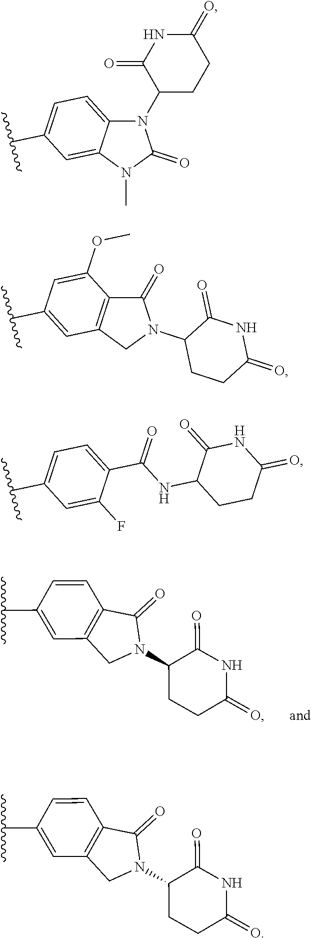

9. The bifunctional compound of any one of embodiments 1-8, wherein W², W⁵, W⁸, and W¹³ are each independently selected from the group consisting of C and N.
10. The bifunctional compound of any one of embodiments 1-8, wherein ITM has a structure selected from the group consisting of formulae (ITM-Ia)-(ITM-Im):

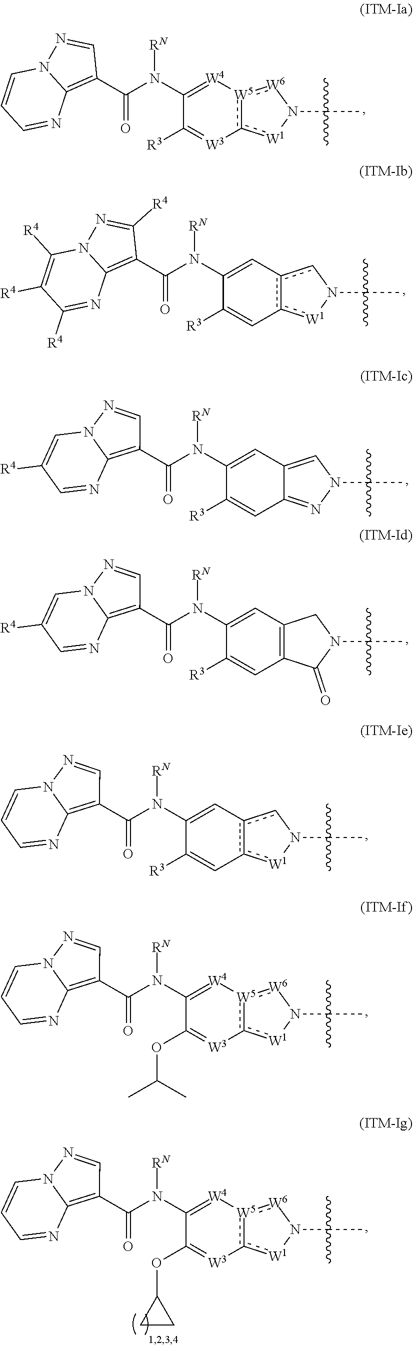

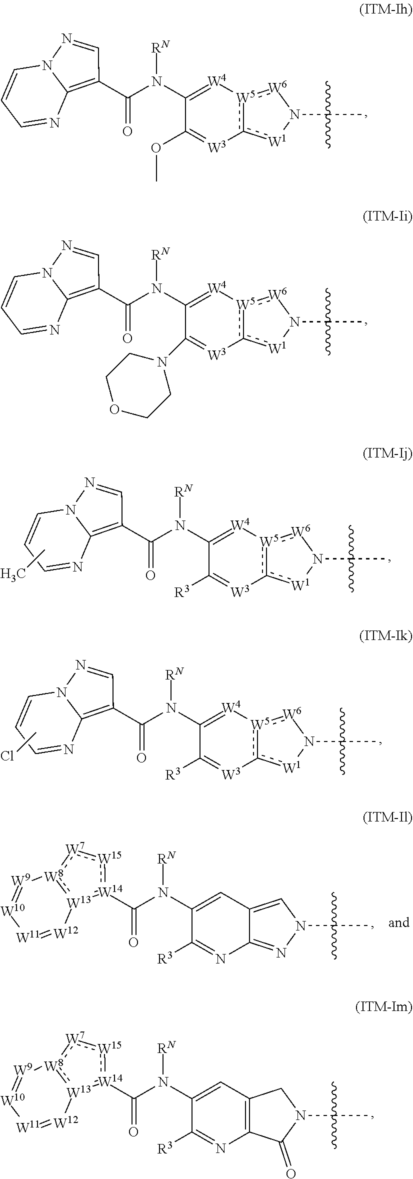

11. The bifunctional compound of embodiment 9, wherein ITM has the structure of Formula (ITM-Ie).
12. The bifunctional compound of any one embodiments 1-11, wherein R^{N} is H or methyl.
13. The bifunctional compound of any one of embodiments 1-12, wherein R³ is —Cl, —F, or C₁₋₆ alkyl optionally substituted with one, two, three, four, or five R⁷.
14. The bifunctional compound of embodiment 13, wherein R⁷ is OH, —Cl, or —F.
15. The bifunctional compound of any one of embodiments 1-14, wherein R³ is selected from the group consisting of

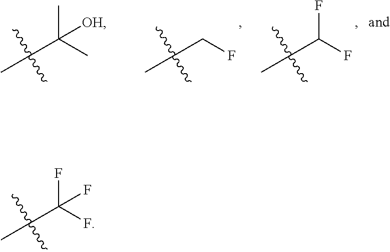

16. The bifunctional compound of any one of embodiments 1-12, wherein R³ is selected from the group consisting of OC₁₋₆ alkyl, OC₃₋₇ cycloalkyl, and OC₁₋₆ alkyl-C₃₋₇ cycloalkyl.
17. The bifunctional compound of embodiment 16, wherein R³ is selected from the group consisting of

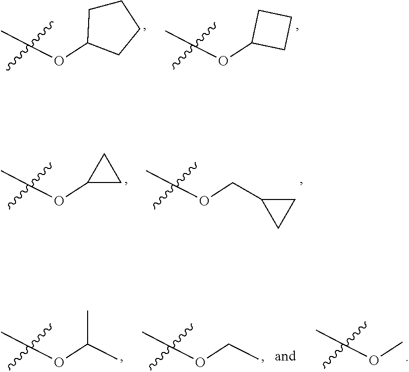

18. The bifunctional compound of any one of embodiments 1-12, wherein R³ is selected from the group consisting of C₁₋₆ alkyl optionally substituted with one, two, three, four, or five R⁷, heterocycloalkyl optionally substituted with R⁷, and OC₃₋₇ cycloalkyl optionally substituted with one, two, three, four, or five R⁷.
19. The bifunctional compound of embodiment 18, wherein R³ is selected from the group consisting of

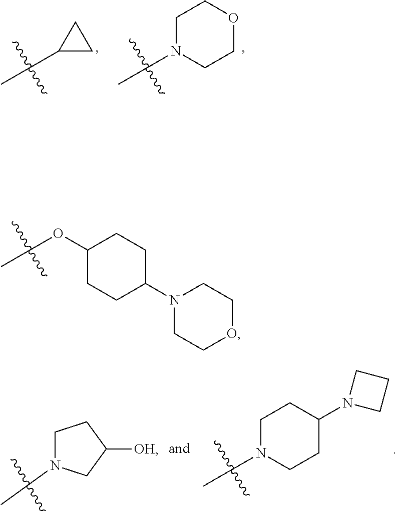

20. The bifunctional compound of any one of embodiments 1-9, wherein ITM has the structure selected from the group consisting of:

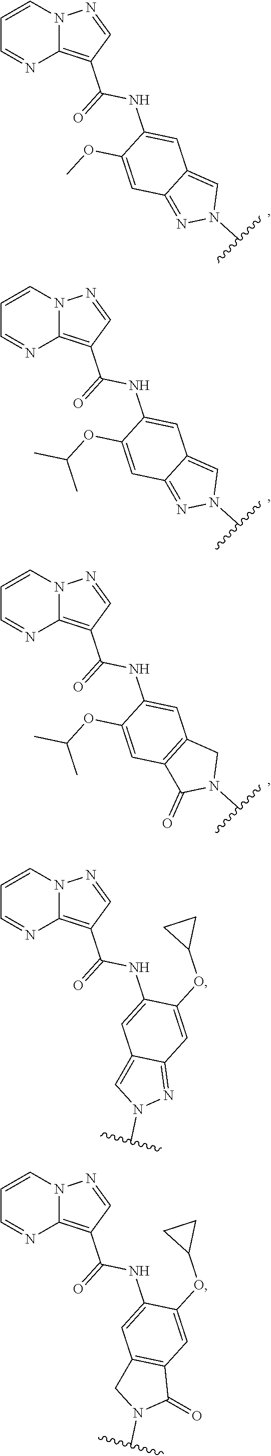 [SMILES: *n1cc2cc(NC(=O)c3cnn4cccnc34)c(OC)cc2n1 ; *n1cc2cc(NC(=O)c3cnn4cccnc34)c(OC(C)C)cc2n1 ; *N1Cc2cc(NC(=O)c3cnn4cccnc34)c(OC(C)C)cc2C1=O ; *n1cc2cc(NC(=O)c3cnn4cccnc34)c(OC3CC3)cc2n1 ; *N1Cc2cc(NC(=O)c3cnn4cccnc34)c(OC3CC3)cc2C1=O]

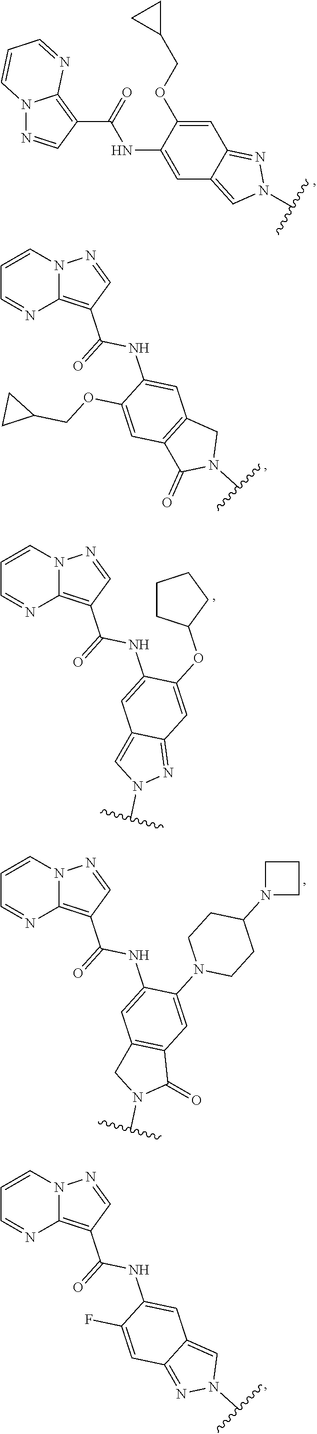 [SMILES: *n1cc2cc(NC(=O)c3cnn4cccnc34)c(OCC3CC3)cc2n1 ; *N1Cc2cc(NC(=O)c3cnn4cccnc34)c(OCC3CC3)cc2C1=O ; *n1cc2cc(NC(=O)c3cnn4cccnc34)c(OC3CCCC3)cc2n1 ; *N1Cc2cc(NC(=O)c3cnn4cccnc34)c(N3CCC(N4CCC4)CC3)cc2C1=O ; *n1cc2cc(NC(=O)c3cnn4cccnc34)c(F)cc2n1]

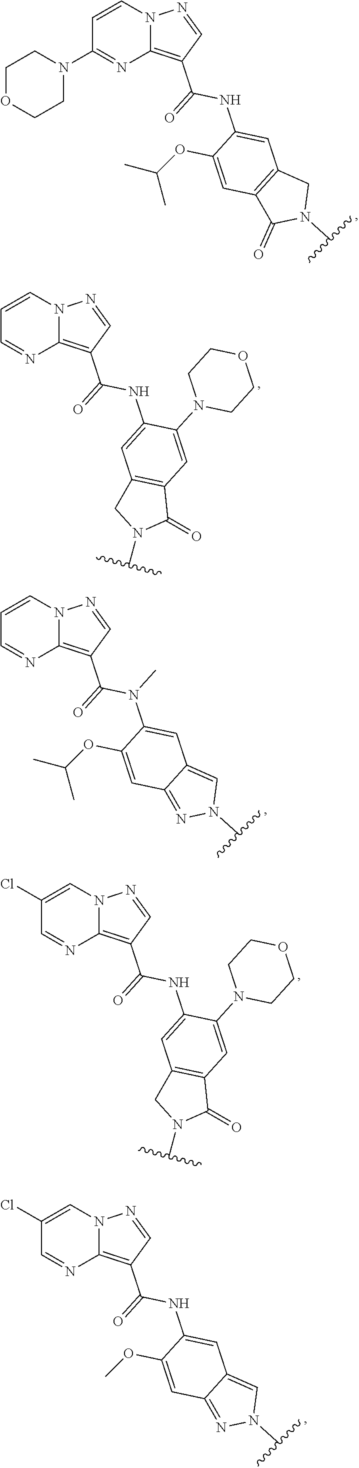 [SMILES: *N1Cc2cc(NC(=O)c3cnn4ccc(N5CCOCC5)nc34)c(OC(C)C)cc2C1=O ; *N1Cc2cc(NC(=O)c3cnn4cccnc34)c(N3CCOCC3)cc2C1=O ; *n1cc2cc(N(C)C(=O)c3cnn4cccnc34)c(OC(C)C)cc2n1 ; *N1Cc2cc(NC(=O)c3cnn4cc(Cl)cnc34)c(N3CCOCC3)cc2C1=O ; *n1cc2cc(NC(=O)c3cnn4cc(Cl)cnc34)c(OC)cc2n1]

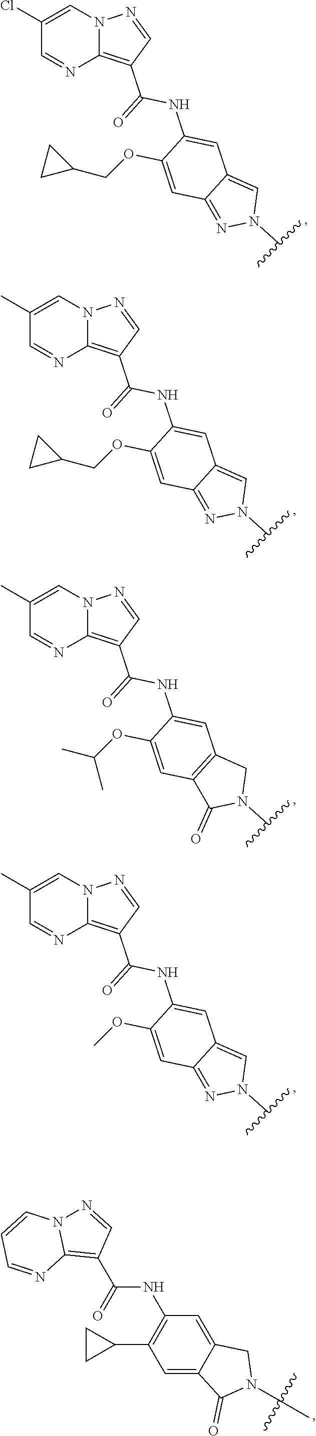 [SMILES: *n1cc2cc(NC(=O)c3cnn4cc(Cl)cnc34)c(OCC3CC3)cc2n1 ; *n1cc2cc(NC(=O)c3cnn4cc(C)cnc34)c(OCC3CC3)cc2n1 ; *N1Cc2cc(NC(=O)c3cnn4cc(C)cnc34)c(OC(C)C)cc2C1=O ; *n1cc2cc(NC(=O)c3cnn4cc(C)cnc34)c(OC)cc2n1 ; *N1Cc2cc(NC(=O)c3cnn4cccnc34)c(C3CC3)cc2C1=O]

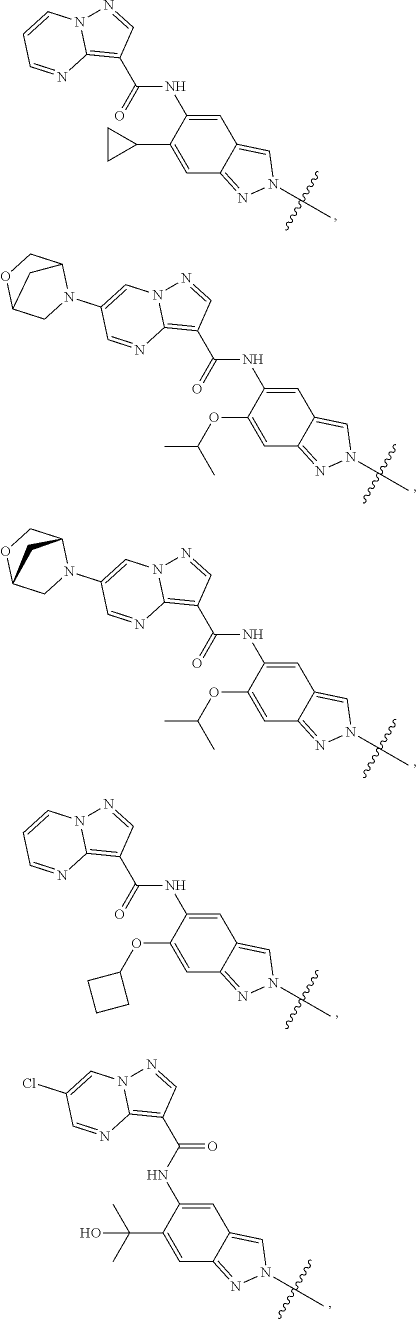 [SMILES: *n1cc2cc(NC(=O)c3cnn4cccnc34)c(C3CC3)cc2n1 ; *n1cc2cc(NC(=O)c3cnn4cc(N5CC6CC5CO6)cnc34)c(OC(C)C)cc2n1 ; *n1cc2cc(NC(=O)c3cnn4cc(N5C[C@H]6C[C@@H]5CO6)cnc34)c(OC(C)C)cc2n1 ; *n1cc2cc(NC(=O)c3cnn4cccnc34)c(OC3CCC3)cc2n1 ; *n1cc2cc(NC(=O)c3cnn4cc(Cl)cnc34)c(C(C)(C)O)cc2n1]

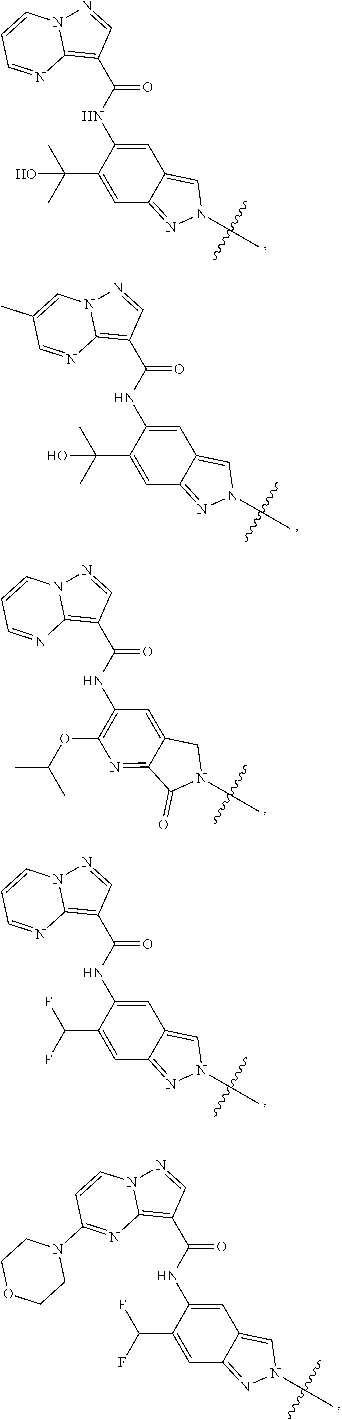 [SMILES: *n1cc2cc(NC(=O)c3cnn4cccnc34)c(C(C)(C)O)cc2n1 ; *n1cc2cc(NC(=O)c3cnn4cc(C)cnc34)c(C(C)(C)O)cc2n1 ; *N1Cc2cc(NC(=O)c3cnn4cccnc34)c(OC(C)C)nc2C1=O ; *n1cc2cc(NC(=O)c3cnn4cccnc34)c(C(F)F)cc2n1 ; *n1cc2cc(NC(=O)c3cnn4ccc(N5CCOCC5)nc34)c(C(F)F)cc2n1]

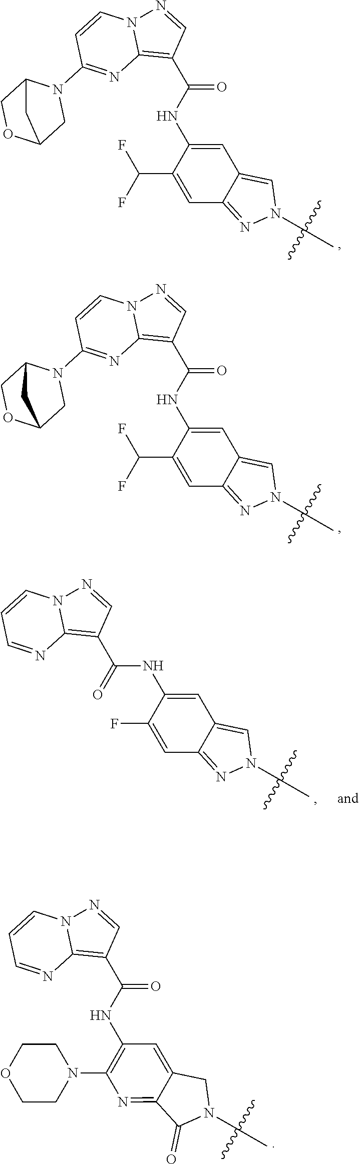 [SMILES: *n1cc2cc(NC(=O)c3cnn4ccc(N5CC6CC5CO6)nc34)c(C(F)F)cc2n1 ; *n1cc2cc(NC(=O)c3cnn4ccc(N5C[C@H]6C[C@@H]5CO6)nc34)c(C(F)F)cc2n1 ; *n1cc2cc(NC(=O)c3cnn4cccnc34)c(F)cc2n1 ; *N1Cc2cc(NC(=O)c3cnn4cccnc34)c(N3CCOCC3)nc2C1=O]

21. The bifunctional compound of any one of embodiments 1-9, wherein ITM has the structure of selected from the group consisting of formulae (ITM-I-1)-(ITM-I-5):

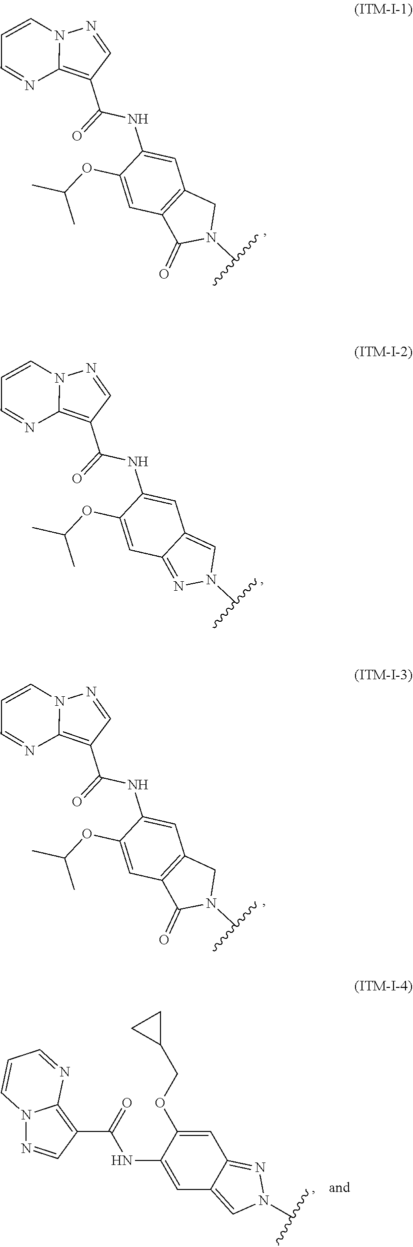 [SMILES: *N1Cc2cc(NC(=O)c3cnn4cccnc34)c(OC(C)C)cc2C1=O ; *n1cc2cc(NC(=O)c3cnn4cccnc34)c(OC(C)C)cc2n1 ; *N1Cc2cc(NC(=O)c3cnn4cccnc34)c(OC(C)C)cc2C1=O ; *n1cc2cc(NC(=O)c3cnn4cccnc34)c(OCC3CC3)cc2n1]

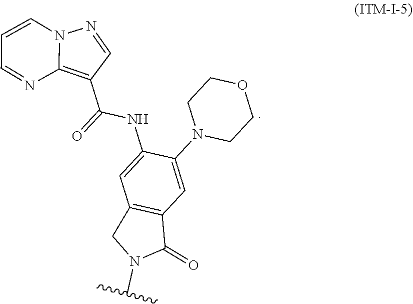 [SMILES: *N1Cc2cc(NC(=O)c3cnn4cccnc34)c(N3CCOCC3)cc2C1=O]

22. The bifunctional compound of any one of embodiments 1-21, wherein LNK is selected from the group consisting of —Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—.
23. The bifunctional compound of any one of embodiments 1-22, wherein LNK is

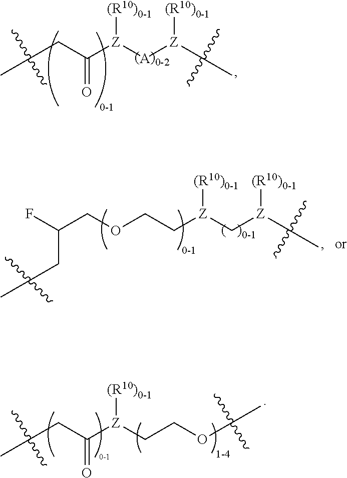

24. The bifunctional compound of any one of embodiments 1-22, wherein LNK is

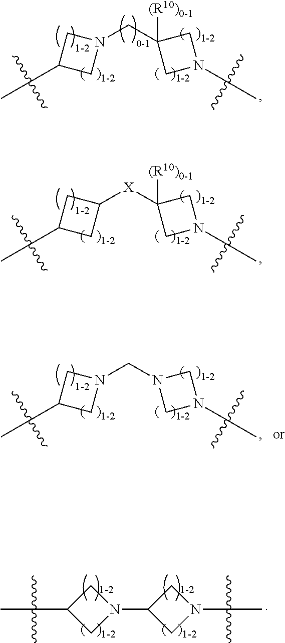

25. The bifunctional compound of embodiment 24, wherein LNK is

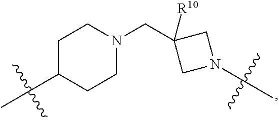

wherein R¹⁰ is H or —F;

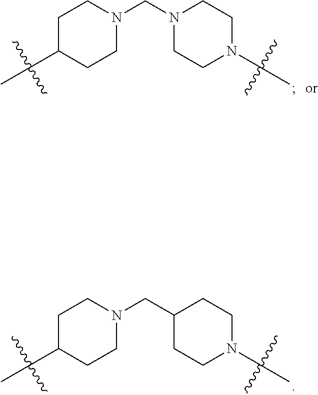 [SMILES: *C1CCN(CN2CCN(*)CC2)CC1 ; *C1CCN(CC2CCN(*)CC2)CC1]

26. The bifunctional compound of any one of embodiments 1-22, wherein LNK is

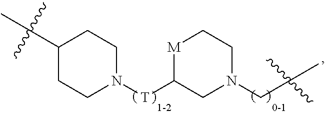

wherein M is CH₂ or O; and each T is independently selected from the group consisting of CH₂, —CH₂—O—, —CH₂—O—CH₂—, —CH₂CH₂—O—, —CH₂CH₂—O—CH₂CH₂—, —CH₂—O—CH₂CH₂—, —CH₂CH₂—O—CH₂—, —C(═O)—O—, and —C(═O)—NH—.
27. The bifunctional compound of any one of embodiments 1-21, wherein LNK is selected from group consisting of:

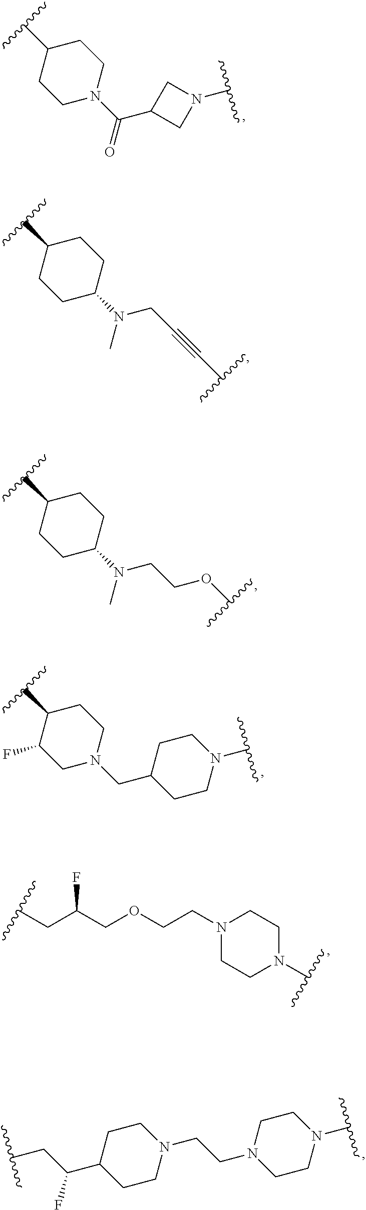 [SMILES: *C1CCN(C(=O)C2CN(*)C2)CC1 ; *C#CCN(C)[C@H]1CC[C@H](*)CC1 ; *OCCN(C)[C@H]1CC[C@H](*)CC1 ; *[C@H]1CCN(CC2CCN(*)CC2)C[C@@H]1F ; *C[C@@H](F)COCCN1CCN(*)CC1 ; *C[C@@H](F)C1CCN(CCN2CCN(*)CC2)CC1]

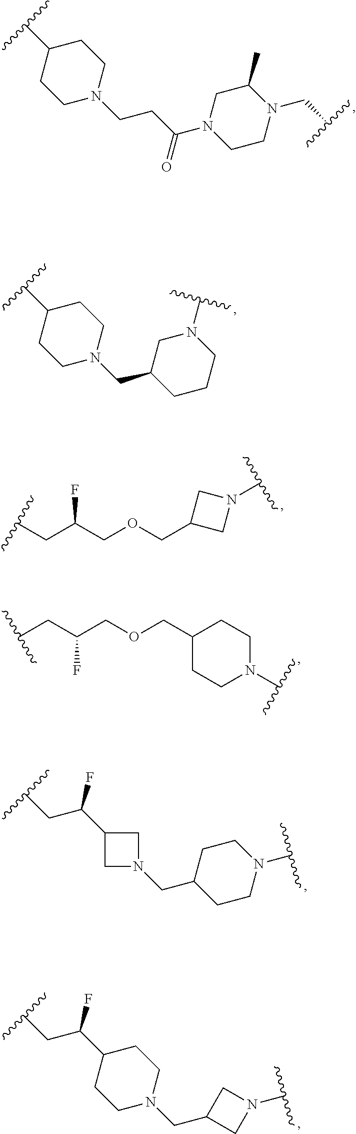

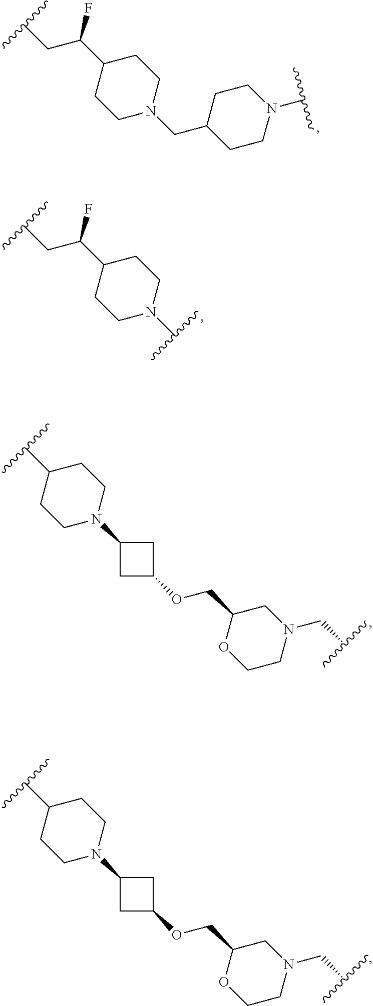

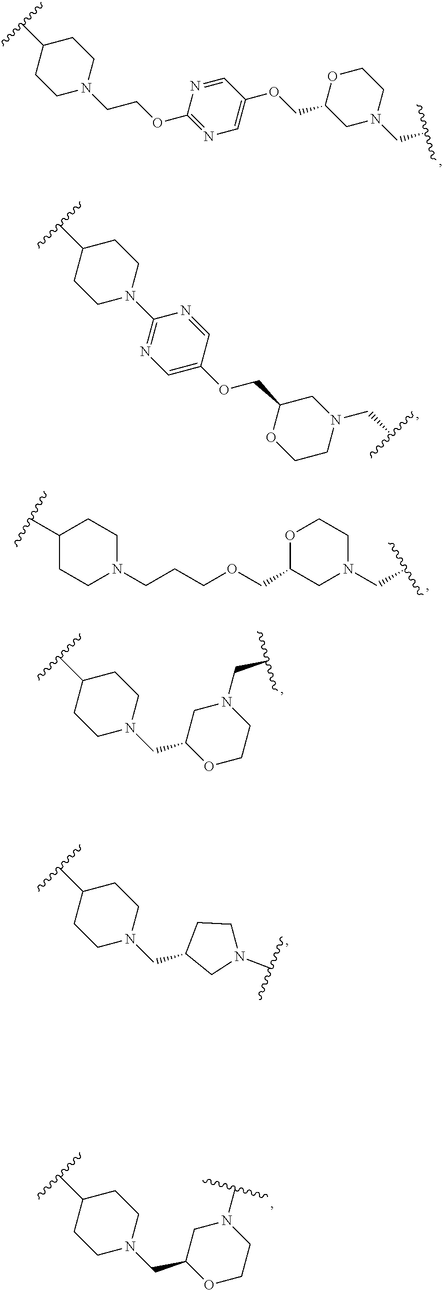

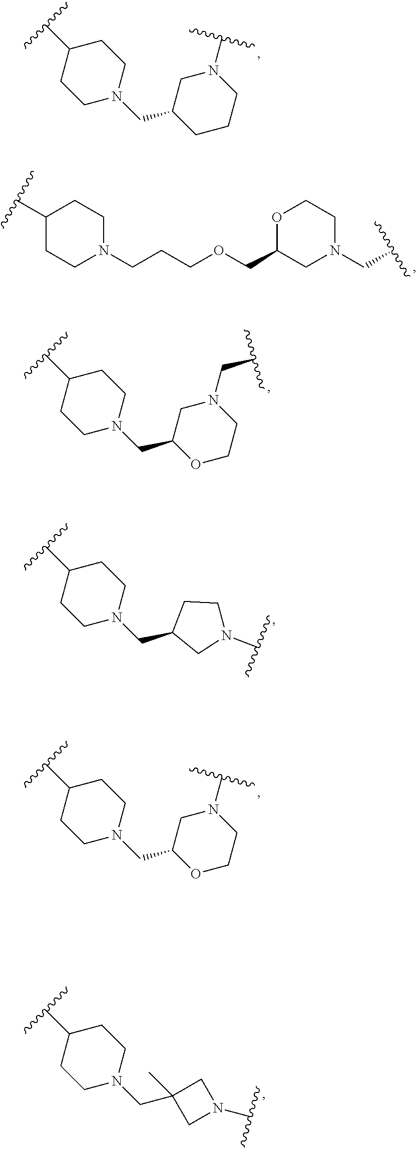

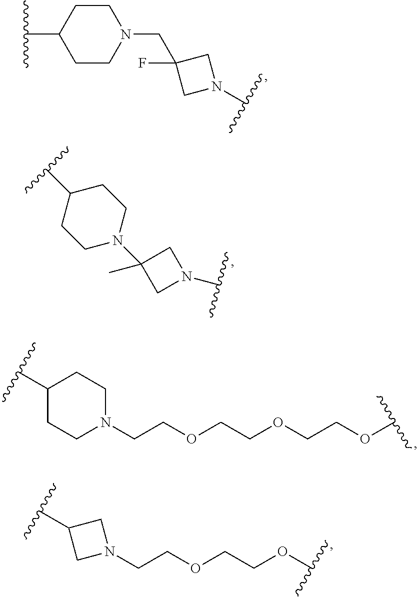 [SMILES: *C1CCN(CC2(F)CN(*)C2)CC1 ; *C1CCN(C2(C)CN(*)C2)CC1 ; *OCCOCCOCCN1CCC(*)CC1 ; *OCCOCCN1CC(*)C1]

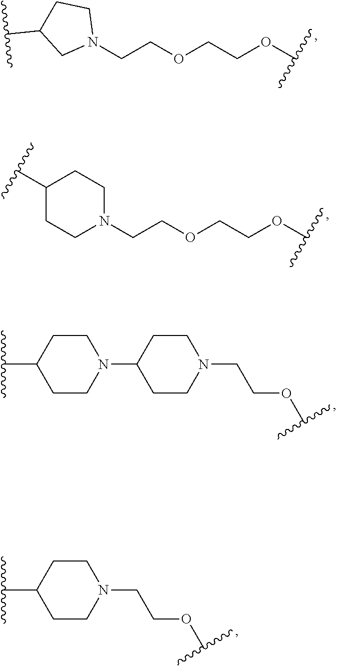 [SMILES: *OCCOCCN1CCC(*)C1 ; *OCCOCCN1CCC(*)CC1 ; *OCCN1CCC(N2CCC(*)CC2)CC1 ; *OCCN1CCC(*)CC1]

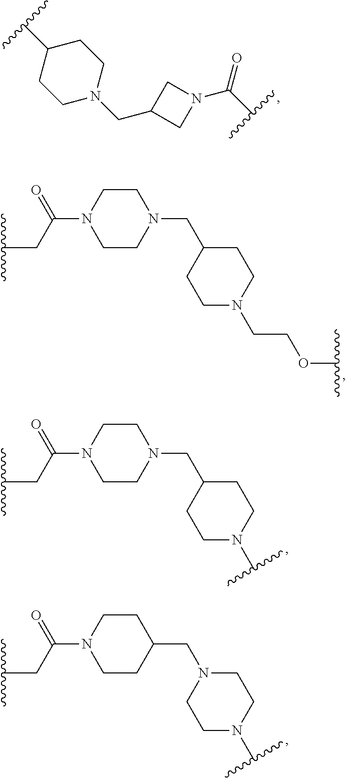 [SMILES: *C(=O)N1CC(CN2CCC(*)CC2)C1 ; *CC(=O)N1CCN(CC2CCN(CCO*)CC2)CC1 ; *CC(=O)N1CCN(CC2CCN(*)CC2)CC1 ; *CC(=O)N1CCC(CN2CCN(*)CC2)CC1]

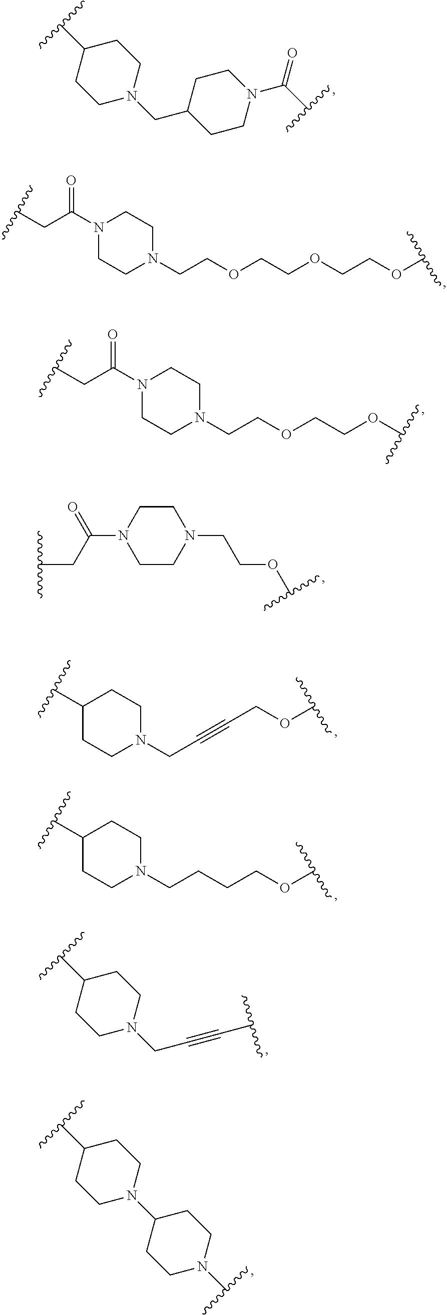

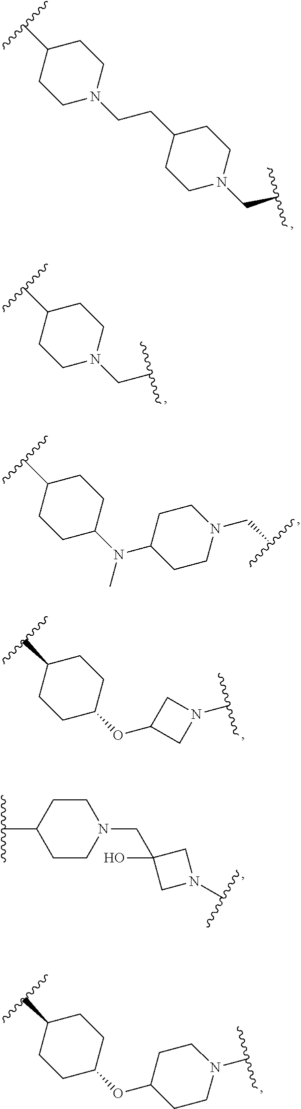

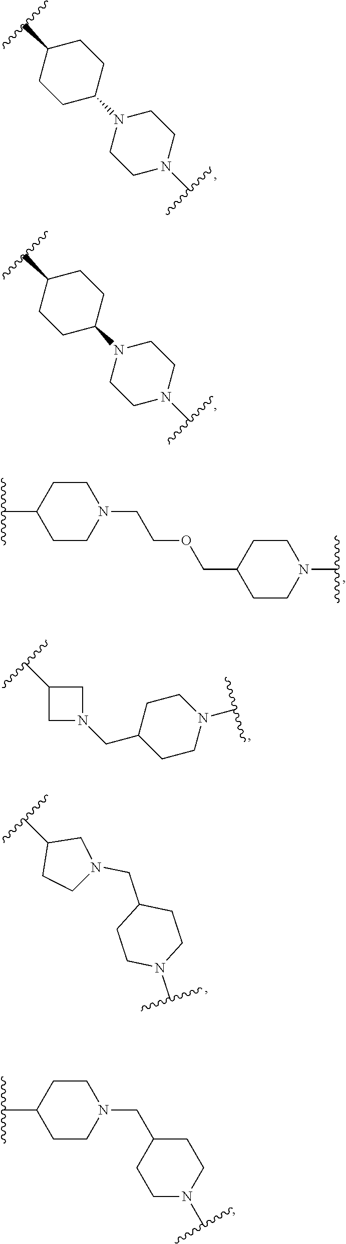 [SMILES: *N1CCN([C@H]2CC[C@H](*)CC2)CC1 ; *N1CCN([C@H]2CC[C@@H](*)CC2)CC1 ; *C1CCN(CCOCC2CCN(*)CC2)CC1 ; *C1CN(CC2CCN(*)CC2)C1 ; *C1CCN(CC2CCN(*)CC2)C1 ; *C1CCN(CC2CCN(*)CC2)CC1]

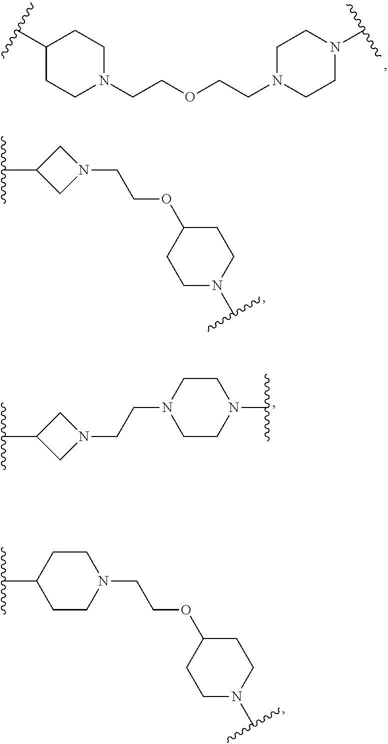 [SMILES: *C1CCN(CCOCCN2CCN(*)CC2)CC1 ; *C1CN(CCOC2CCN(*)CC2)C1 ; *C1CN(CCN2CCN(*)CC2)C1 ; *C1CCN(CCOC2CCN(*)CC2)CC1]

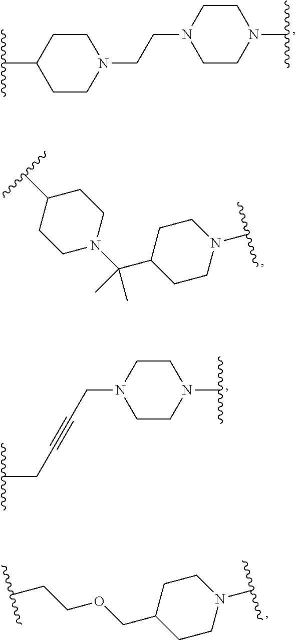 [SMILES: *C1CCN(CCN2CCN(*)CC2)CC1 ; *C1CCN(C(C)(C)C2CCN(*)CC2)CC1 ; *CC#CCN1CCN(*)CC1 ; *CCOCC1CCN(*)CC1]

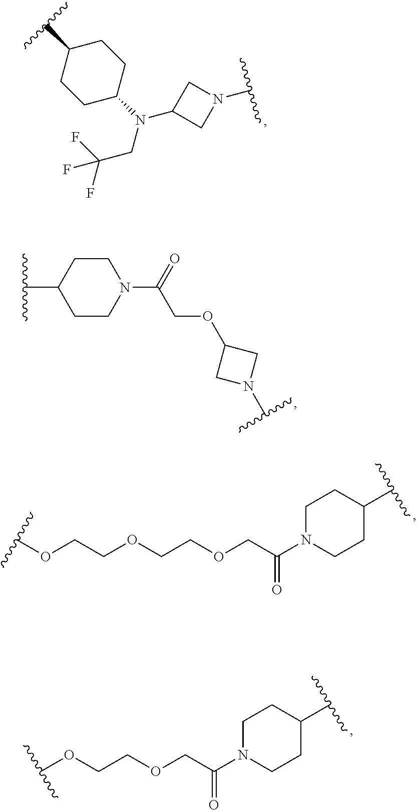 [SMILES: *N1CC(N(CC(F)(F)F)[C@H]2CC[C@H](*)CC2)C1 ; *C1CCN(C(=O)COC2CN(*)C2)CC1 ; *OCCOCCOCC(=O)N1CCC(*)CC1 ; *OCCOCC(=O)N1CCC(*)CC1]

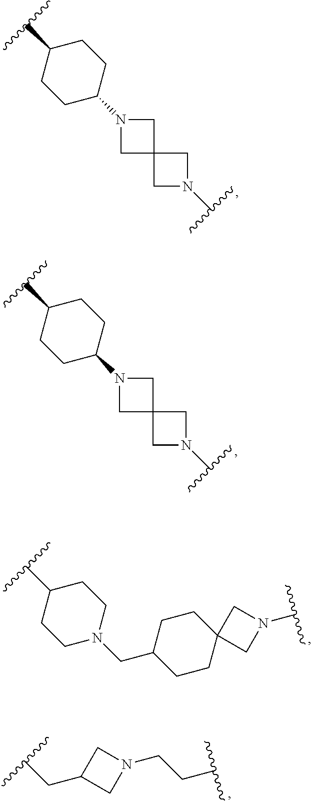 [SMILES: *N1CC2(C1)CN([C@H]1CC[C@H](*)CC1)C2 ; *N1CC2(C1)CN([C@H]1CC[C@@H](*)CC1)C2 ; *C1CCN(CC2CCC3(CC2)CN(*)C3)CC1 ; *CCN1CC(C*)C1]

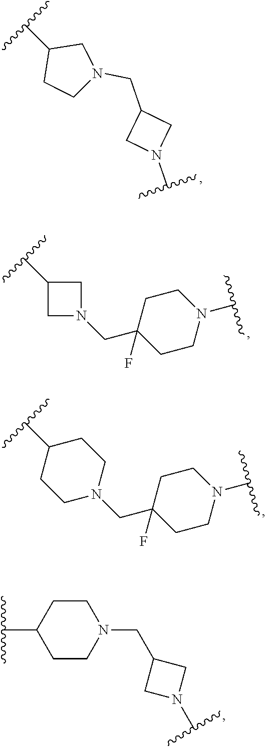 [SMILES: *C1CCN(CC2CN(*)C2)C1 ; *C1CN(CC2(F)CCN(*)CC2)C1 ; *C1CCN(CC2(F)CCN(*)CC2)CC1 ; *C1CCN(CC2CN(*)C2)CC1]

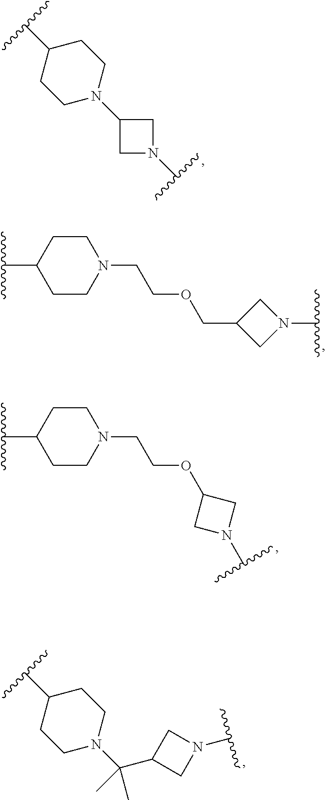 [SMILES: *C1CCN(C2CN(*)C2)CC1 ; *C1CCN(CCOCC2CN(*)C2)CC1 ; *C1CCN(CCOC2CN(*)C2)CC1 ; *C1CCN(C(C)(C)C2CN(*)C2)CC1]

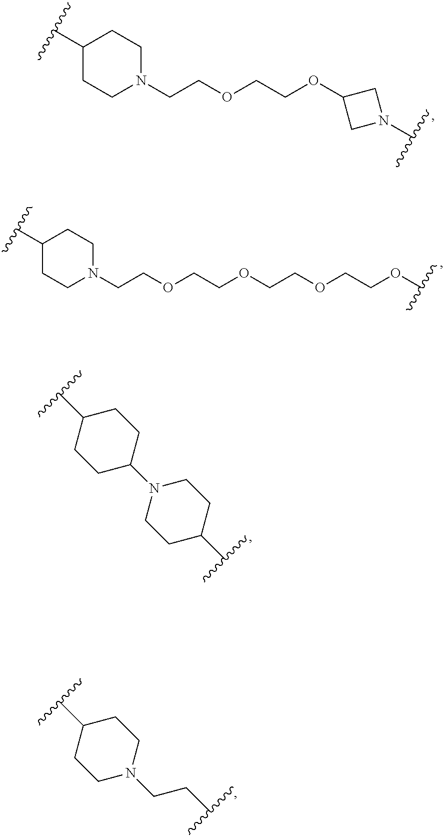 [SMILES: *C1CCN(CCOCCOC2CN(*)C2)CC1 ; *OCCOCCOCCOCCN1CCC(*)CC1 ; *C1CCC(N2CCC(*)CC2)CC1 ; *CCN1CCC(*)CC1]

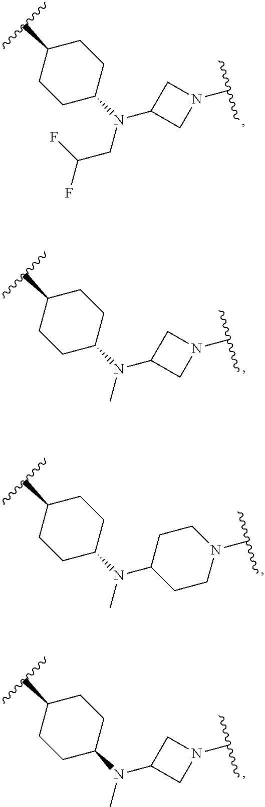 [SMILES: *N1CC(N(CC(F)F)[C@H]2CC[C@H](*)CC2)C1 ; *N1CC(N(C)[C@H]2CC[C@H](*)CC2)C1 ; *N1CCC(N(C)[C@H]2CC[C@H](*)CC2)CC1 ; *N1CC(N(C)[C@H]2CC[C@@H](*)CC2)C1]

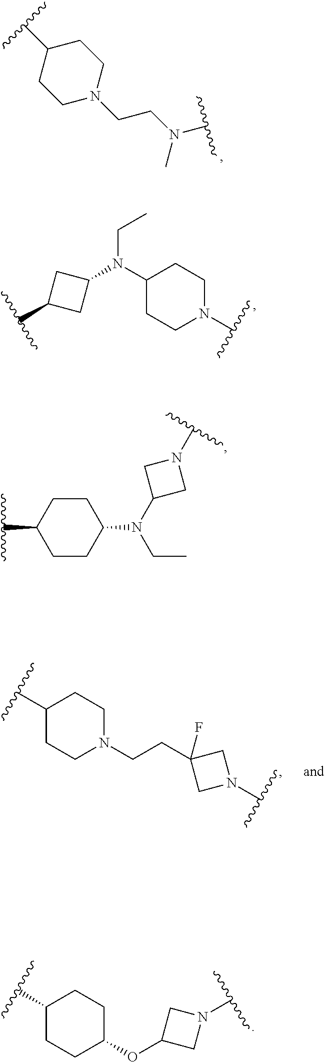 [SMILES: *C1CCN(CCN(*)C)CC1 ; *N1CCC(N(CC)[C@H]2C[C@H](*)C2)CC1 ; *N1CC(N(CC)[C@H]2CC[C@H](*)CC2)C1 ; *C1CCN(CCC2(F)CN(*)C2)CC1 ; *N1CC(O[C@H]2CC[C@@H](*)CC2)C1]

28. The bifunctional compound of embodiment 1, wherein the compound is selected from the group consisting of the compounds in any of Tables 1, 2, 3, 4, 5, 6, 7, 8, 9, 10, 11, 12, 13, and pharmaceutically acceptable salts, solvates, enantiomers, stereoisomers, or isotopic derivatives thereof.
29. The bifunctional compound of embodiment 1, wherein the compound is selected from the group consisting of the compounds in any of Tables 1, 2, 3, 4, 5, 6, 7, 8, 9, 10, 11, 12, 13, and pharmaceutically acceptable salts thereof.
30. The bifunctional compound of embodiment 1, wherein the compound is selected from the group consisting of the compounds in any of Tables 1, 2, 3, 4, 5, 6, 7, 8, 9, 10, 11, 12, and 13.
31. A pharmaceutical composition comprising the bifunctional compound of any one of embodiments 1-29 and one or more pharmaceutically acceptable excipients.
32. A pharmaceutical composition of embodiment 30 further comprising an additional bioactive agent, wherein the bioactive agent is an anti-cancer agent, anti-inflammatory agent, anti-neurodegenerative agent, or anti-immunological agent.
33. A method of treating a disease or disorder in a subject, the method comprising administering to the subject in need thereof a therapeutically effective amount of a bifunctional compound of any one of embodiments 1-29 or a therapeutically effective amount of the pharmaceutical composition of embodiment 30 or 31.
34. The method of embodiment 32, wherein the disease or disorder is a neurodegenerative disease or disorder, an inflammatory disease or disorder, a immunological disease or disorder, and/or a cancer associated with signaling through signaling pathways regulated by IRAK-4 and/or the myddosome complex.
35. The method of embodiment 33, wherein the neurodegenerative and/or neuroinflammatory disease or disorder is Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, or graft versus host disease.
36. The method of embodiment 33, wherein the inflammatory disease or disorder is ocular allergy, conjunctivitis, keratoconjunctivitis sicca, vernal conjunctivitis, allergic rhinitis, autoimmune hematological disorders (e.g., hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Stevens-Johnson syndrome, idiopathic sprue, autoimmune inflammatory bowel disease (e.g., ulcerative colitis and Crohn's disease), irritable bowel syndrome, celiac disease, periodontitis, hyaline membrane disease, kidney disease, glomerular disease, alcoholic liver disease, multiple sclerosis, inflammation associated with or caused by multiple sclerosis, endocrine opthalmopathy, Grave's disease, sarcoidosis, alveolitis, chronic hypersensitivity pneumonitis, primary biliary cirrhosis, uveitis (anterior and posterior), Sjogren's syndrome, interstitial lung fibrosis, psoriatic arthritis, systemic juvenile idiopathic arthritis, nephritis, vasculitis, diverticulitis, interstitial cystitis, glomerulonephritis (e.g., including idiopathic nephrotic syndrome or minimal change nephropathy), chronic granulomatous disease, endometriosis, leptospirosis renal disease, glaucoma, retinal disease, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma, acute lung injury, acute respiratory distress syndrome, eosinophilia, hypersensitivities, anaphylaxis, fibrositis, gastritis, gastroenteritis, nasal sinusitis, ocular allergy, silica induced diseases, chronic obstructive pulmonary disease (COPD), cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, juvenile rheumatoid arthritis, dermatomyositis, encephalitis, endocarditis, endometritis, enteritis, enterocolitis, epicondylitis, epididymitis, fasciitis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, vasculitis, vulvitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, Cryopyrin Associated Periodic Syndrome (CAPS), or osteoarthritis.
37. The method of embodiment 33, wherein the immunological disease or disorder is multiple sclerosis, rheumatoid arthritis, spondyloarthropathy, systemic lupus erythematosus, antibody-mediated inflammatory or autoimmune disease, graft-versus-host disease, sepsis, diabetes, psoriasis, atheroma, atherosclerosis, Sjogren's syndrome, progressive systemic sclerosis, scleroderma, acute coronary syndrome, ischemia reperfusion, Crohn's disease, endometriosis, glomerulonephritis, myasthenia gravis, idiopathic pulmonary fibrosis.
38. The method of embodiment 33, wherein the cancer is squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitt's Lymphoma, B-cell ALL, Philadelphia chromosome positive ALL, or Philadelphia chromosome positive CML.

1. A bifunctional compound having the structure of Formula (I): 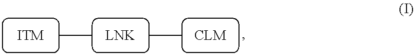
or a pharmaceutically acceptable salt thereof,
wherein:
(a) ITM is a moiety having the structure of formula ITM-I: 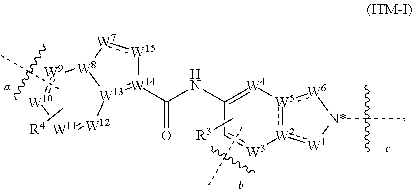
wherein: [symbol]
is a single bond or a double bond;
N* is N or NH;
W¹ and W⁶ are each independently C═O, CH, O, N, CH₂, CR¹, or NR¹;
W², W³, W⁴, W⁵, W⁷, W⁸, W⁹, W¹⁰, W¹¹, W¹², W¹³, W¹⁴, and W¹⁵ are each independently C, N, C(R¹)ₘ, or NR¹;
R¹ is H, —Cl, —F, —Br, —I, N(R²)₂; linear or branched C₁₋₆ alkyl, C₁₋₆ haloalkyl, 3- to 7-membered heterocycloalkyl, or 5- to 6-membered heteroaryl optionally substituted with one, two, three, four, or five R⁶;
R³ and R⁴ are each independently selected from H, —Cl, —F, —Br, —I, linear or branched C₁₋₆ alkyl optionally substituted with one, two, three, four, or five R⁷, OC₁₋₆ alkyl, OC₁₋₆ alkyl-C₃₋₇ cycloalkyl, monocyclic C₃₋₇ cycloalkyl optionally substituted with one, two, three, four, or five R⁷, fused bicyclic C₃₋₇ cycloalkyl optionally substituted with one, two, three, four, or five R⁷, bridged bicyclic C₃₋₇ cycloalkyl optionally substituted with one, two, three, four, or five R⁷, spiro-fused bicyclic C₃₋₇ cycloalkyl optionally substituted with one, two, three, four, or five R⁷, monocyclic heterocycloalkyl optionally substituted with one, two, three, four, or five R⁷, fused bicyclic heterocycloalkyl optionally substituted with one, two, three, four, or five R⁷, bridged bicyclic heterocycloalkyl optionally substituted with one, two, three, four, or five R⁷, spiro-fused bicyclic heterocycloalkyl optionally substituted with one, two, three, four, or five R⁷, OC₃₋₇ cycloalkyl optionally substituted with one, two, three, four, or five R⁷, and C₁₋₆alkyl-C₃₋₇cycloalkyl optionally substituted with one, two, three, four, or five R⁷;
R^{N} is H, linear or branched C₁₋₆ alkyl, or C₁₋₆ haloalkyl;
each R² is independently selected from H and linear or branched C₁₋₆ alkyl;
each R⁶ is independently selected from OH, OC₁₋₆ alkyl, and heterocycloalkyl;
each R⁷ is independently selected from —Cl, —F, —Br, —I, NH₂, CN, CF₃, linear or branched C₁₋₆ alkyl, OC₁₋₆ alkyl, NH(C₁₋₆ alkyl), N(C₁₋₆ alkyl)(C₁₋₆ alkyl), C₆₋₁₀ aryl, 5- to 6-membered heteroaryl, C₃-C₇ cycloalkyl, and 3- to 7-membered heterocycloalkyl;
m is 1 or 2; and
ITM is connected to LNK through one of 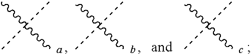
(b) LNK is a chemical linking moiety that covalently couples the ITM to the CLM, having the structure: [symbol]
L [symbol] ₙ
wherein:
each L is independently selected from X, Y, and Z;
each X is independently selected from 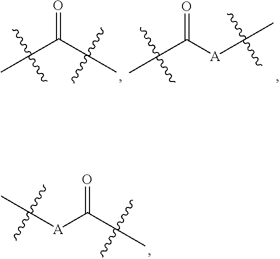
and -A-, wherein each A is independently selected from C(R^{8A})₂, NR⁸, and O;
each R^{8A} is independently selected from H, linear or branched C₁₋₆ alkyl optionally substituted with —Cl, —F, —OH, NH₂, CN, or CF₃, O—C₁₋₆ alkyl optionally substituted with —Cl, —F, —OH, NH₂, CN, or CF₃, C₁₋₆ haloalkyl, NH₂, CN, CF₃, —Cl, —F, —Br, —I, and OH;
each R⁸ is independently selected from H, linear or branched C₁₋₆ alkyl optionally substituted with —Cl, —F, —OH, NH₂, CN, or CF₃, O—C₁₋₆ alkyl, and C₁₋₆ haloalkyl;
each Y is independently selected from 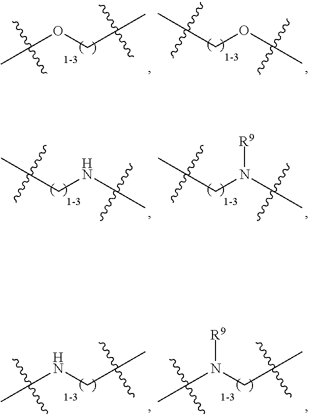
C₁₋₆ alkylene, C₂₋₆ alkenylene, and C₂₋₆ alkynylene;
wherein each Y is independently optionally substituted with one, two, three, four, or five R⁹;
each R⁹ is independently selected from linear or branched C₁₋₆ alkyl optionally substituted with —Cl, —F, —OH, NH₂, CN, or CF₃; O—C₁₋₆ alkyl optionally substituted with —Cl, —F, —OH, NH₂, CN, or CF₃, C₁₋₆ haloalkyl, NH, CN, CF₃, —Cl, —F, —Br, —I, and OH;
each Z is independently selected from monocyclic C₄₋₁₀ cycloalkylene, fused bicyclic C₄₋₁₀ cycloalkylene, bridged bicyclic C₄₋₁₀ cycloalkylene, or spiro-fused bicyclic C₄₋₁₀ cycloalkylene, 4-6 membered heterocycloalkylene, C₆-C₁₀ arylene, and 5-6 membered heteroarylene;
wherein each Z is independently optionally substituted with one, two, three, four, or five R¹⁰;
each R¹⁰ is independently selected from —Cl, —F, —Br, —I, linear or branched C₁₋₆ alkyl optionally substituted with —C, —F, —OH, NH₂, CN, or CF₃, O—C₁₋₆ alkyl optionally substituted with —C, —F, —OH, NH₂, CN, or CF₃, C₁₋₆ haloalkyl, NH₂, CN, CF₃, and OH; and
n is 1, 2, 3, 4, 5, or 6;
(c) CLM is a moiety having a structure selected from formulae CLM-I, CLM-II, and CLM-III: 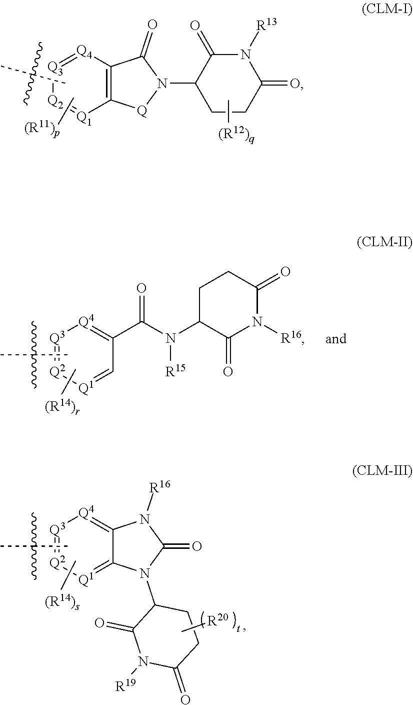
wherein:
Q¹, Q², Q³, and Q⁴ are each independently selected from CH, C, and N;
each R¹¹ is independently selected from —Cl, —F, —Br, —I, CN, CF₃, NH₂, NH(C₁₋₆ alkyl), N(C₁₋₆ alkyl)(C₆ alkyl), OH, O—C₁₋₆ alkyl optionally substituted with R²¹, and linear or branched C₁₋₆ alkyl optionally substituted with R²¹;
p is 0, 1, 2, 3, or 4;
Q is selected from CH₂, CH(C₁₋₆ alkyl), C═O, SO₂, NH, and N(C₁₋₆ alkyl);
each R¹² is independently selected from —Cl, —F, —Br, —I, O—C₁₋₆ alkyl optionally substituted with R²¹, NH₂, NH(C₁₋₆ alkyl), N(C₁₋₆ alkyl)(C₁₋₆ alkyl), OH, CN, CF₃, and linear or branched C₁₋₆ alkyl optionally substituted with R²¹;
q is 0, 1, 2, or 3;
R¹³ is selected from H, OH, linear or branched C₁₋₆ alkyl optionally substituted with R²¹, C(O)C₁₋₆ alkyl, C(O)NH—C₁₋₆ alkyl and C(O)OC₁₋₆ alkyl;
each R¹⁴ is independently selected from —Cl, —F, —Br, —I, NH₂, NH(C₁₋₆ alkyl), N(C₁₋₆ alkyl)(C₁₋₆ alkyl), OH, CN, CF₃, O—C₁₋₆ alkyl optionally substituted with R²¹, and linear or branched C₁₋₆ alkyl optionally substituted with R²¹;
r is 0, 1, 2, 3, or 4;
R¹⁵ is selected from H and linear or branched C₁₋₆ alkyl optionally substituted with R²¹;
R¹⁶ is selected from H, OH, linear or branched C₁₋₆ alkyl optionally substituted with R²¹, C(O)C₁₋₆ alkyl, C(O)NH—C₁₋₆ alkyl, and C(O)OC₁₋₆ alkyl;
s is 0, 1, 2, or 3;
R¹⁸ is selected from H and linear or branched C₁₋₆ alkyl optionally substituted with R²¹;
R¹⁹ is selected from H, OH, linear or branched C₁₋₆ alkyl optionally substituted with R²¹, C(O)C₁₋₆ alkyl, C(O)NH—C₁₋₆ alkyl, and C(O)OC₁₋₆ alkyl;
each R²⁰ is independently selected from —Cl, —F, —Br, —I, O—C₁₋₆ alkyl optionally substituted with R²¹, NH₂, NH(C₁₋₆ alkyl), N(C₁₋₆ alkyl)(C₁₋₆ alkyl), OH, CN, CF₃, and linear or branched C₁₋₆ alkyl optionally substituted with R²¹;
t is 0, 1, 2, or 3;
each R²¹ is independently selected from —Cl, —F, —OH, NH₂, CN, and CF₃; and
———— is the point of attachment to LNK.

2. The bifunctional compound of claim 1, wherein CLM has a structure CLM-I.

3. The bifunctional compound of claim 2, wherein q is 0 or R¹³ or methyl.

4. The bifunctional compound of claim 1, wherein CLM has a structure CLM-II.

5. The bifunctional compound of claim 4, wherein R¹⁵ is H or methyl and R¹⁶ is H or methyl.

6. The bifunctional compound of claim 1, wherein CLM has a structure CLM-III.

7. The bifunctional compound of claim 6, wherein t is 0 or R¹⁹ is methyl.

8. The bifunctional compound of claim 1, wherein CLM has the structure selected from:

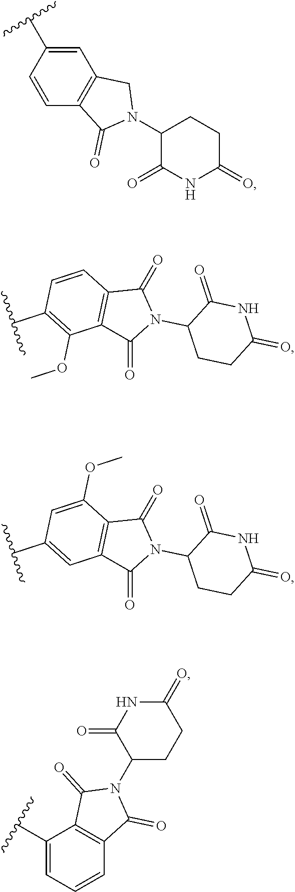 [SMILES: *c1ccc2c(c1)CN(C1CCC(=O)NC1=O)C2=O ; *c1ccc2c(c1OC)C(=O)N(C1CCC(=O)NC1=O)C2=O ; *c1cc(OC)c2c(c1)C(=O)N(C1CCC(=O)NC1=O)C2=O ; *c1cccc2c1C(=O)N(C1CCC(=O)NC1=O)C2=O]

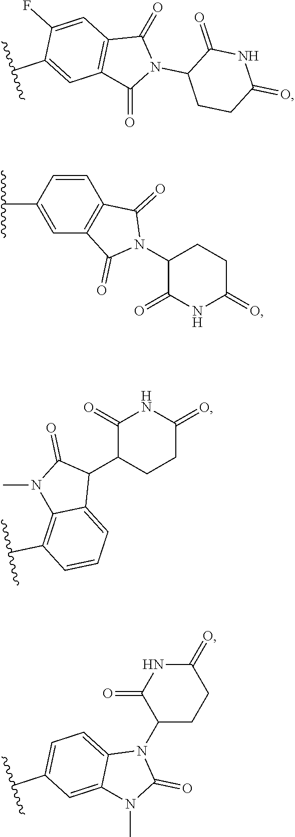 [SMILES: *c1cc2c(cc1F)C(=O)N(C1CCC(=O)NC1=O)C2=O ; *c1ccc2c(c1)C(=O)N(C1CCC(=O)NC1=O)C2=O ; *c1cccc2c1N(C)C(=O)C2C1CCC(=O)NC1=O ; *c1ccc2c(c1)n(C)c(=O)n2C1CCC(=O)NC1=O]

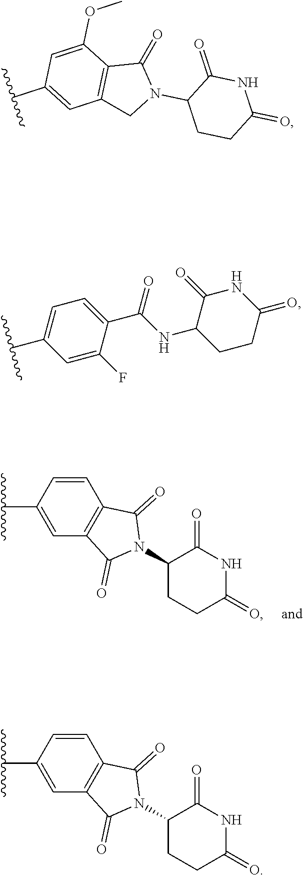 [SMILES: *c1cc2c(c(OC)c1)C(=O)N(C1CCC(=O)NC1=O)C2 ; *c1ccc(C(=O)NC2CCC(=O)NC2=O)c(F)c1 ; *c1ccc2c(c1)C(=O)N([C@@H]1CCC(=O)NC1=O)C2=O ; *c1ccc2c(c1)C(=O)N([C@H]1CCC(=O)NC1=O)C2=O]

9. The bifunctional compound of claim 1, wherein W², W⁵, W⁸, and W¹³ are each independently C or N.

10. The bifunctional compound of claim 1, wherein ITM has a structure selected from formulae (ITM-Ia)-(ITM-Im):

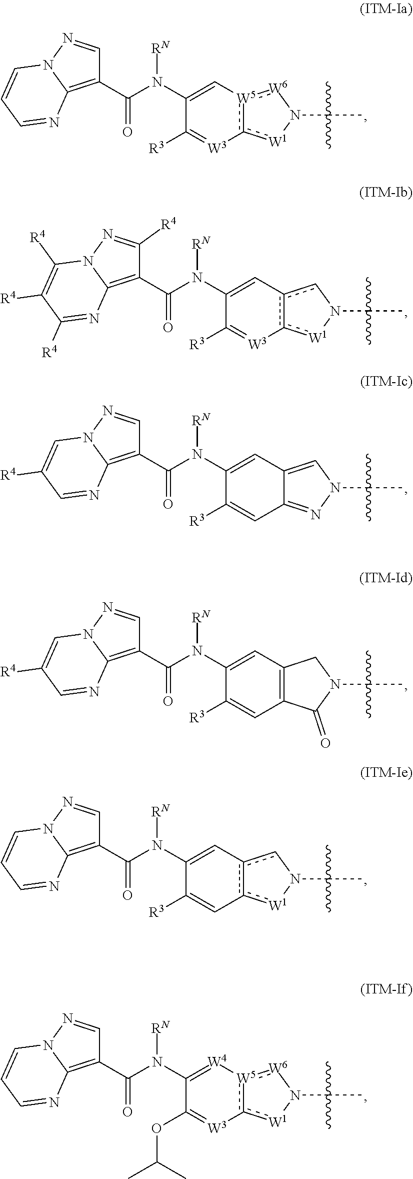

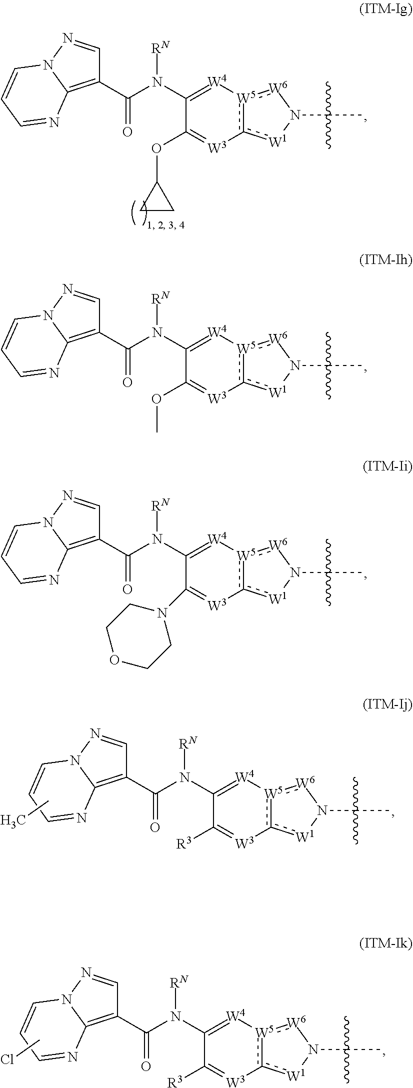

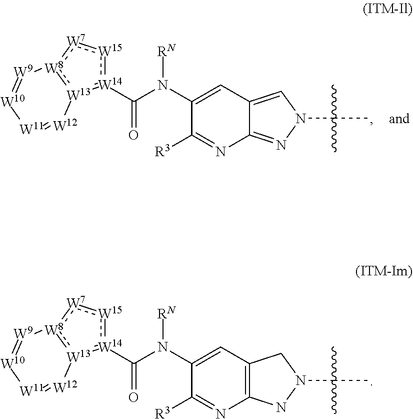

11. The bifunctional compound of claim 9, wherein ITM has the structure of Formula (ITM-Ie).

12. The bifunctional compound of claim 1, wherein R^{N} is H or methyl.

13. The bifunctional compound of claim 1, wherein R³ is —Cl, —F, or C₁₋₆ alkyl optionally substituted with one, two, three, four, or five R⁷.

14. The bifunctional compound of claim 13, wherein R⁷ is —Cl or —F.

15. The bifunctional compound of claim 1, wherein R³ is selected from

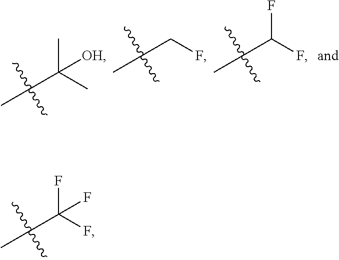

16. The bifunctional compound of claim 1, wherein R³ is selected from OC₁₋₆ alkyl, OC₃₋₇ cycloalkyl, and OC₁₋₆ alkyl-C₃₋₇ cycloalkyl.

17. The bifunctional compound of claim 16, wherein R³ is selected from

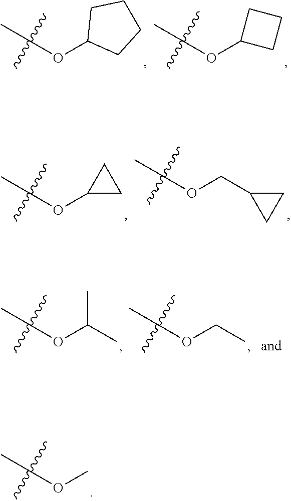

18. The bifunctional compound of claim 1, wherein R³ is selected from C₁₋₆ alkyl optionally substituted with one, two, three, four, or five R⁷, heterocycloalkyl optionally substituted with R⁷, and OC₃₋₇ cycloalkyl optionally substituted with one, two, three, four, or five R⁷.

19. The bifunctional compound of claim 18, wherein R³ is selected from

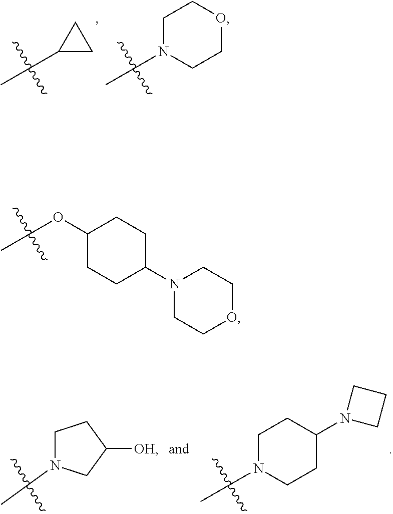

20. The bifunctional compound of claim 1, wherein ITM has the structure selected from:

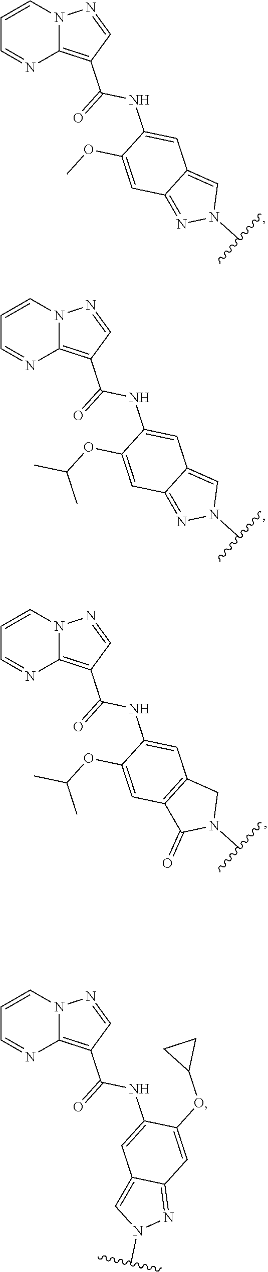 [SMILES: *n1cc2cc(NC(=O)c3cnn4cccnc34)c(OC)cc2n1 ; *n1cc2cc(NC(=O)c3cnn4cccnc34)c(OC(C)C)cc2n1 ; *N1Cc2cc(NC(=O)c3cnn4cccnc34)c(OC(C)C)cc2C1=O ; *n1cc2cc(NC(=O)c3cnn4cccnc34)c(OC3CC3)cc2n1]

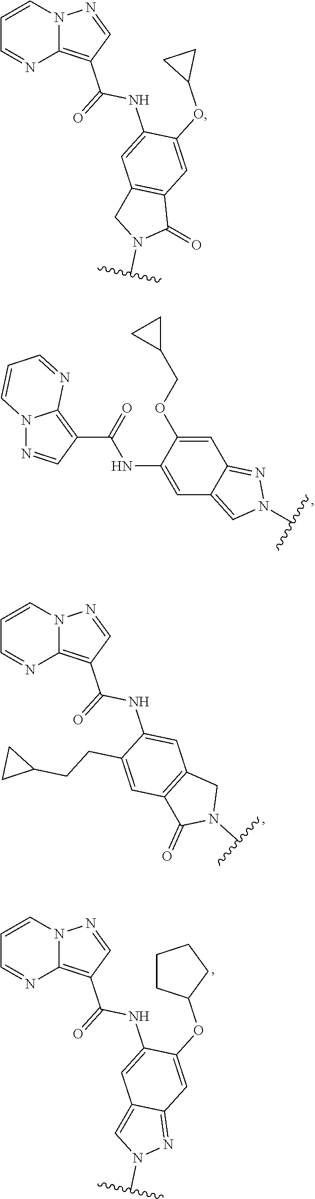 [SMILES: *N1Cc2cc(NC(=O)c3cnn4cccnc34)c(OC3CC3)cc2C1=O ; *n1cc2cc(NC(=O)c3cnn4cccnc34)c(OCC3CC3)cc2n1 ; *N1Cc2cc(NC(=O)c3cnn4cccnc34)c(CCC3CC3)cc2C1=O ; *n1cc2cc(NC(=O)c3cnn4cccnc34)c(OC3CCCC3)cc2n1]

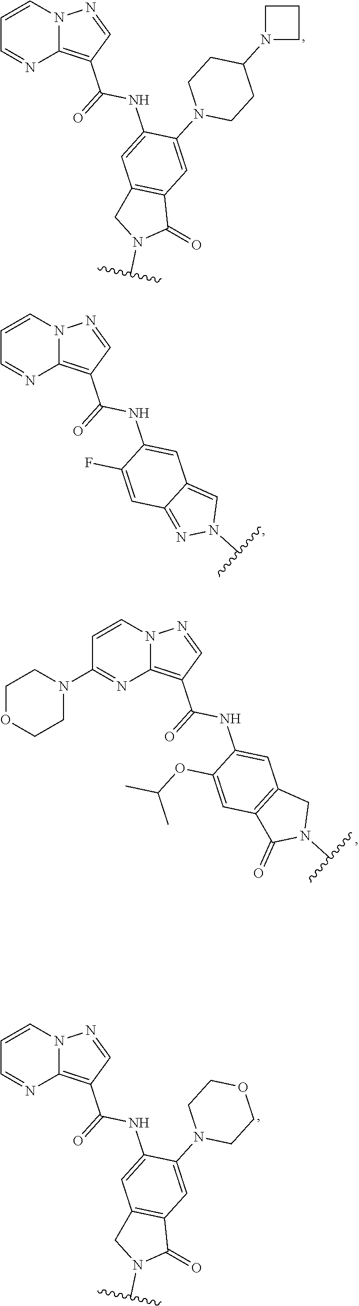 [SMILES: *N1Cc2cc(NC(=O)c3cnn4cccnc34)c(N3CCC(N4CCC4)CC3)cc2C1=O ; *n1cc2cc(NC(=O)c3cnn4cccnc34)c(F)cc2n1 ; *N1Cc2cc(NC(=O)c3cnn4ccc(N5CCOCC5)nc34)c(OC(C)C)cc2C1=O ; *N1Cc2cc(NC(=O)c3cnn4cccnc34)c(N3CCOCC3)cc2C1=O]

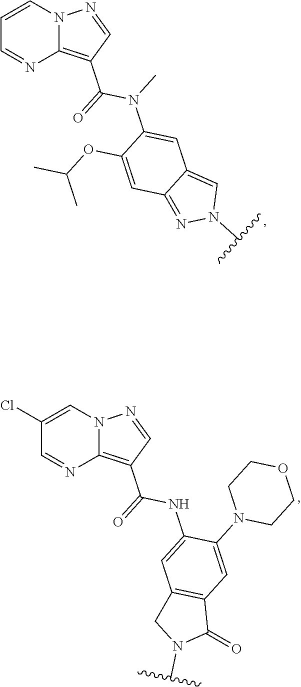 [SMILES: *n1cc2cc(N(C)C(=O)c3cnn4cccnc34)c(OC(C)C)cc2n1 ; *N1Cc2cc(NC(=O)c3cnn4cc(Cl)cnc34)c(N3CCOCC3)cc2C1=O]

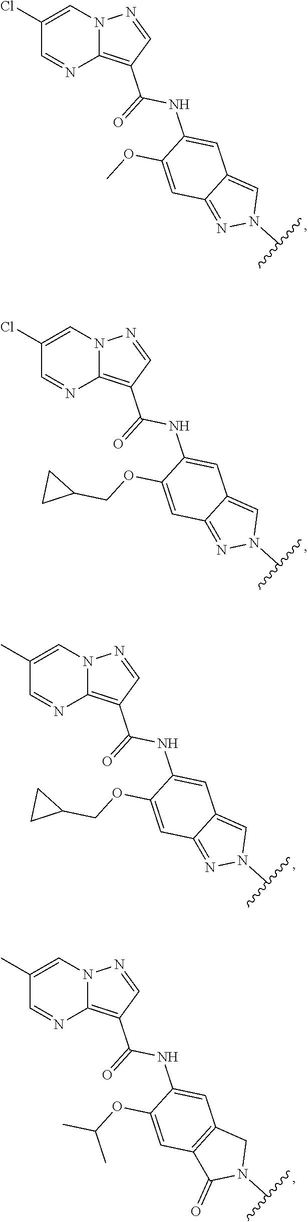 [SMILES: *n1cc2cc(NC(=O)c3cnn4cc(Cl)cnc34)c(OC)cc2n1 ; *n1cc2cc(NC(=O)c3cnn4cc(Cl)cnc34)c(OCC3CC3)cc2n1 ; *n1cc2cc(NC(=O)c3cnn4cc(C)cnc34)c(OCC3CC3)cc2n1 ; *N1Cc2cc(NC(=O)c3cnn4cc(C)cnc34)c(OC(C)C)cc2C1=O]

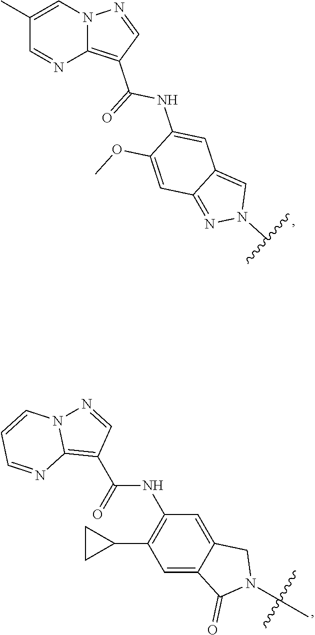 [SMILES: *n1cc2cc(NC(=O)c3cnn4cc(C)cnc34)c(OC)cc2n1 ; *N1Cc2cc(NC(=O)c3cnn4cccnc34)c(C3CC3)cc2C1=O]

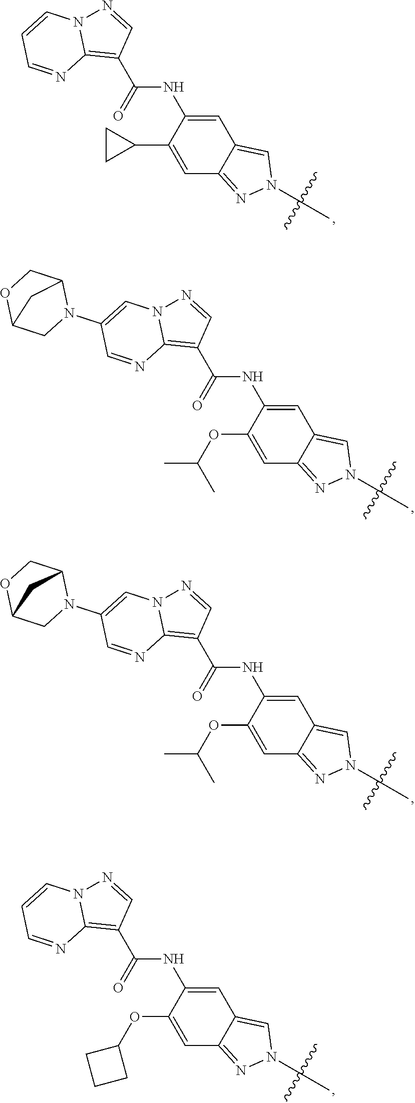 [SMILES: *n1cc2cc(NC(=O)c3cnn4cccnc34)c(C3CC3)cc2n1 ; *n1cc2cc(NC(=O)c3cnn4cc(N5CC6CC5CO6)cnc34)c(OC(C)C)cc2n1 ; *n1cc2cc(NC(=O)c3cnn4cc(N5C[C@H]6C[C@@H]5CO6)cnc34)c(OC(C)C)cc2n1 ; *n1cc2cc(NC(=O)c3cnn4cccnc34)c(OC3CCC3)cc2n1]

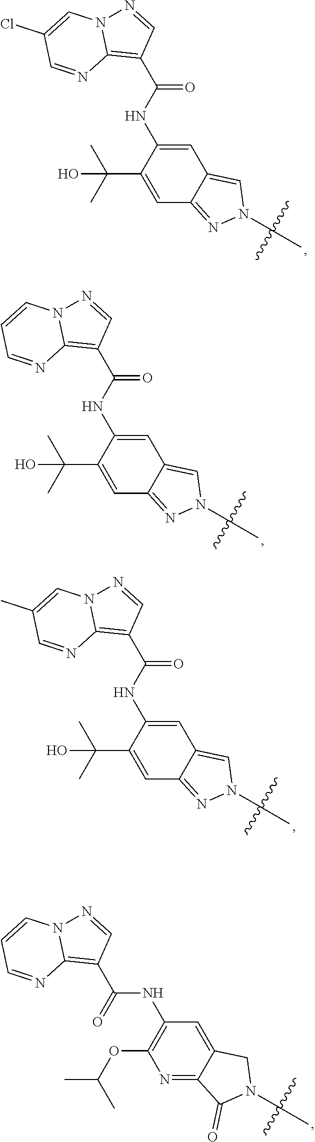 [SMILES: *n1cc2cc(NC(=O)c3cnn4cc(Cl)cnc34)c(C(C)(C)O)cc2n1 ; *n1cc2cc(NC(=O)c3cnn4cccnc34)c(C(C)(C)O)cc2n1 ; *n1cc2cc(NC(=O)c3cnn4cc(C)cnc34)c(C(C)(C)O)cc2n1 ; *N1Cc2cc(NC(=O)c3cnn4cccnc34)c(OC(C)C)nc2C1=O]

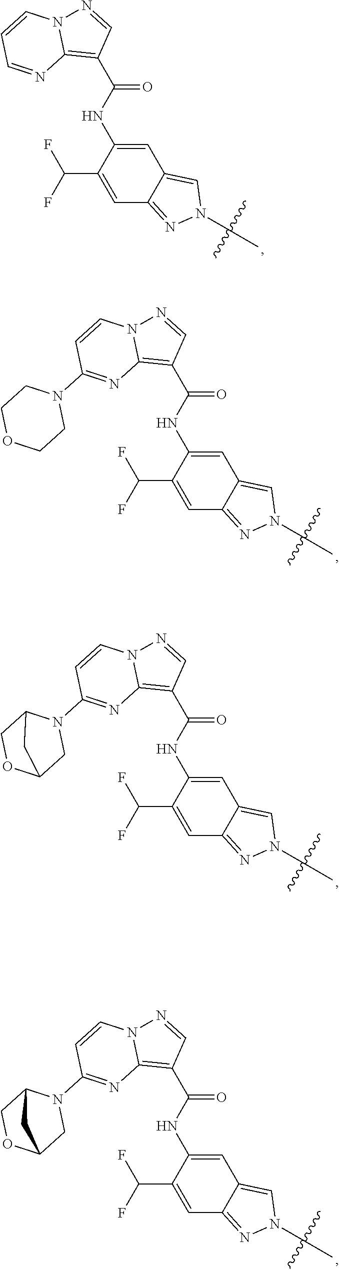 [SMILES: *n1cc2cc(NC(=O)c3cnn4cccnc34)c(C(F)F)cc2n1 ; *n1cc2cc(NC(=O)c3cnn4ccc(N5CCOCC5)nc34)c(C(F)F)cc2n1 ; *n1cc2cc(NC(=O)c3cnn4ccc(N5CC6CC5CO6)nc34)c(C(F)F)cc2n1 ; *n1cc2cc(NC(=O)c3cnn4ccc(N5C[C@H]6C[C@@H]5CO6)nc34)c(C(F)F)cc2n1]

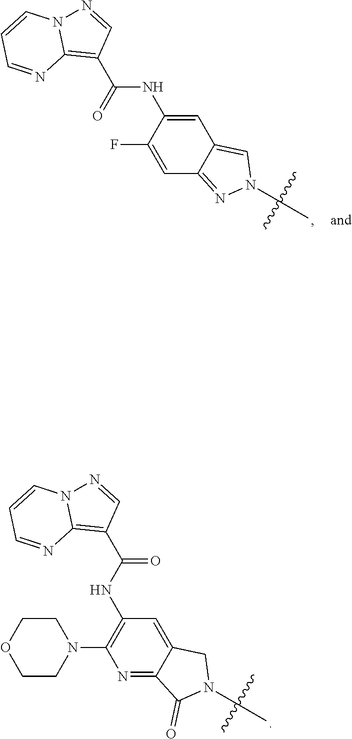 [SMILES: *n1cc2cc(NC(=O)c3cnn4cccnc34)c(F)cc2n1 ; *N1Cc2cc(NC(=O)c3cnn4cccnc34)c(N3CCOCC3)nc2C1=O]

21. The bifunctional compound of claim 1, wherein ITM has the structure of selected from formulae (ITM-I-1)-(ITM-I-5):

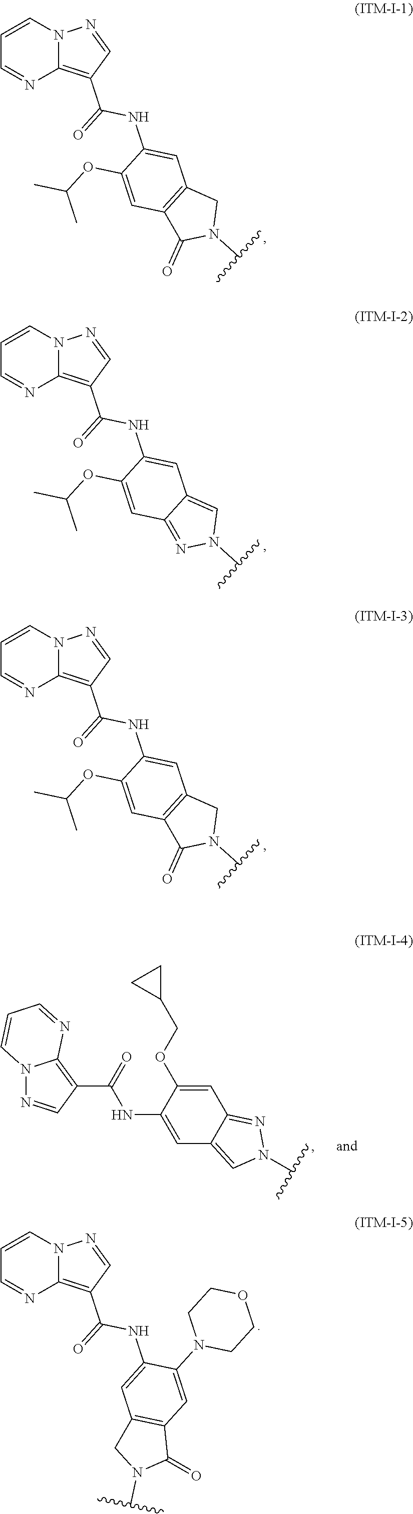 [SMILES: *N1Cc2cc(NC(=O)c3cnn4cccnc34)c(OC(C)C)cc2C1=O ; *n1cc2cc(NC(=O)c3cnn4cccnc34)c(OC(C)C)cc2n1 ; *N1Cc2cc(NC(=O)c3cnn4cccnc34)c(OC(C)C)cc2C1=O ; *n1cc2cc(NC(=O)c3cnn4cccnc34)c(OCC3CC3)cc2n1 ; *N1Cc2cc(NC(=O)c3cnn4cccnc34)c(N3CCOCC3)cc2C1=O]

22. The bifunctional compound of claim 1, wherein LNK is selected from —Z—Y—Z—, —Z—X—Z—, —Z—X—Z—X—, —Z—X—Y—, —Y—Z—Y—Z—, —Y—Z—X—Z—, —Z—Z—, —(X)₀₋₁—(Y)₁₋₅—Z—, —X—Z—(Y)₁₋₄—, and —Z—(Y)₁₋₅—.

23. The bifunctional compound of claim 1, wherein LNK is

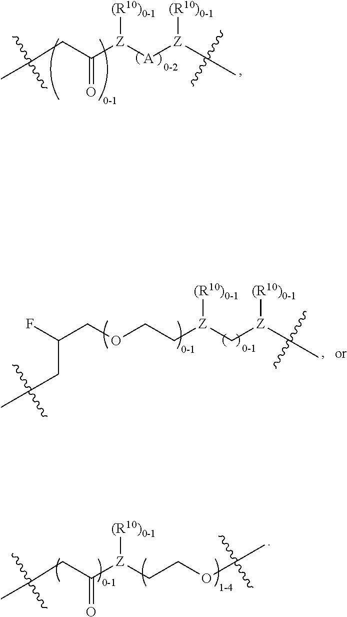

24. The bifunctional compound of claim 1, wherein LNK is

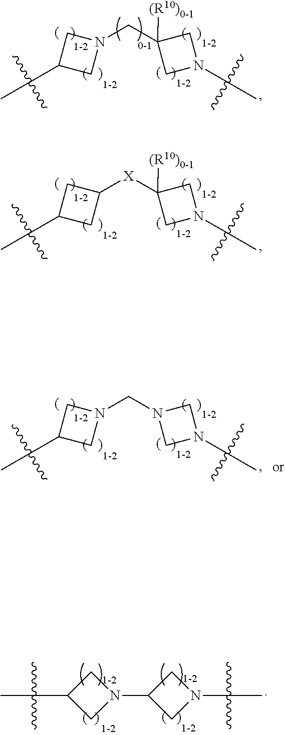

25. The bifunctional compound of claim 24, wherein LNK is 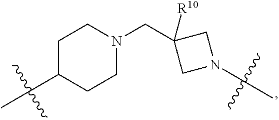
wherein R¹⁰ is H or —F;

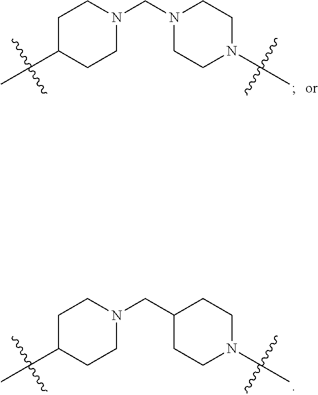 [SMILES: *C1CCN(CN2CCN(*)CC2)CC1 ; *C1CCN(CC2CCN(*)CC2)CC1]

26. The bifunctional compound of claim 1, wherein LNK is 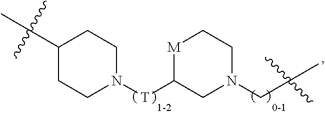
wherein M is CH₂ or O; and each T is independently selected from CH₂, —CH₂—O—, —CH₂—O—CH₂—, —CH₂CH₂—O—, —CH₂CH₂—O—CH₂CH₂—, —CH₂—O—CH₂CH₂—, —CH₂CH₂—O—CH₂—, —C(═O)—O—, and —C(═O)—NH—.

27. The bifunctional compound of claim 1, wherein LNK is selected from:

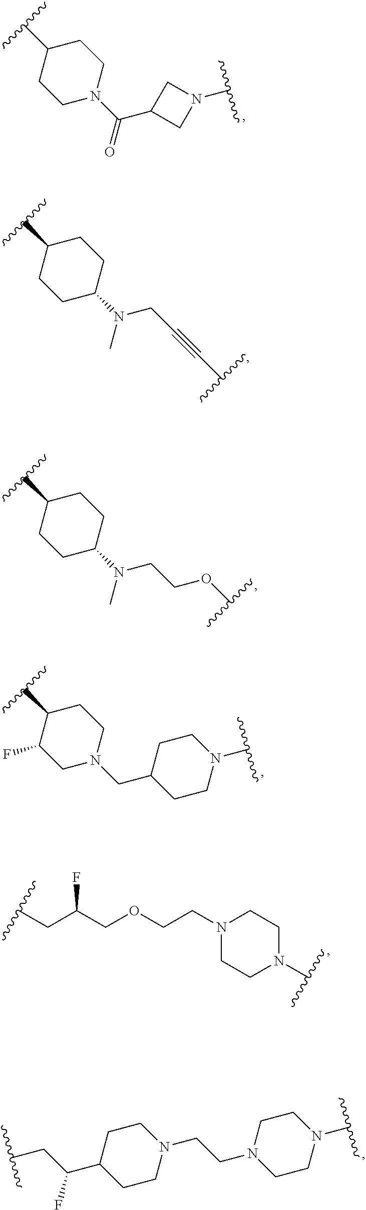 [SMILES: *C1CCN(C(=O)C2CN(*)C2)CC1 ; *C#CCN(C)[C@H]1CC[C@H](*)CC1 ; *OCCN(C)[C@H]1CC[C@H](*)CC1 ; *[C@H]1CCN(CC2CCN(*)CC2)C[C@@H]1F ; *C[C@@H](F)COCCN1CCN(*)CC1 ; *C[C@@H](F)C1CCN(CCN2CCN(*)CC2)CC1]

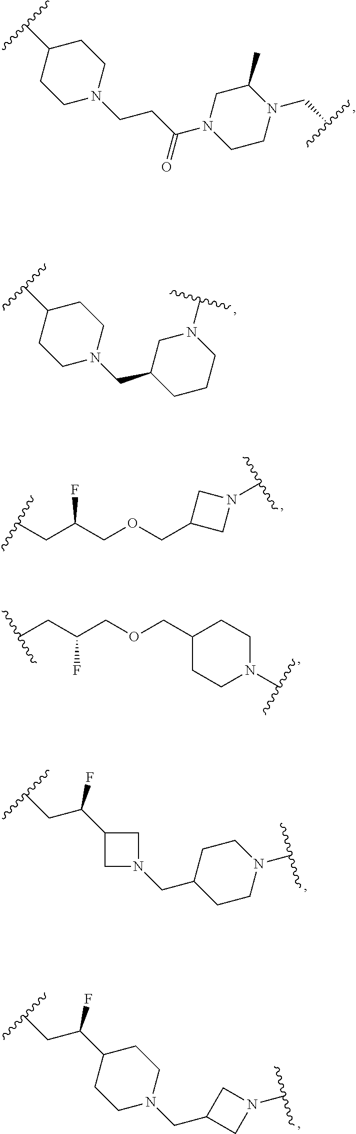

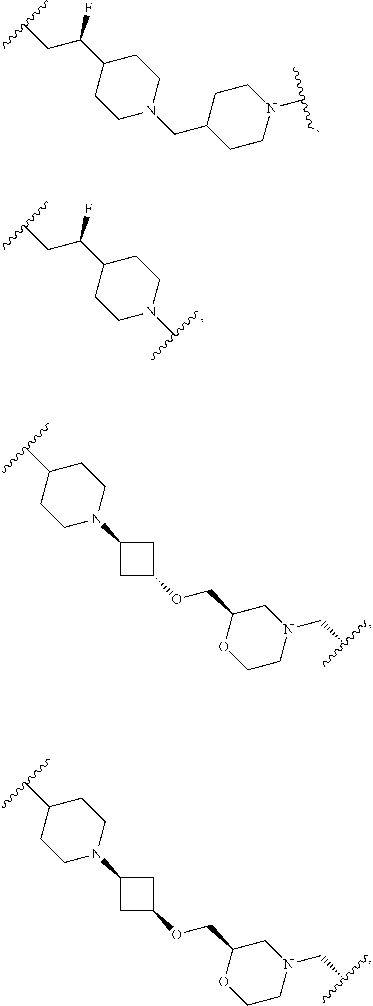

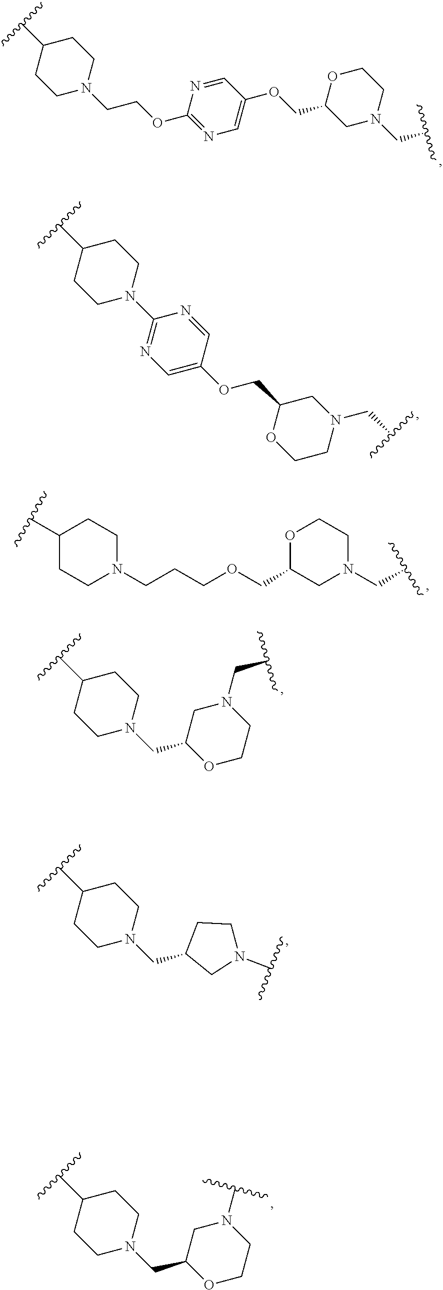

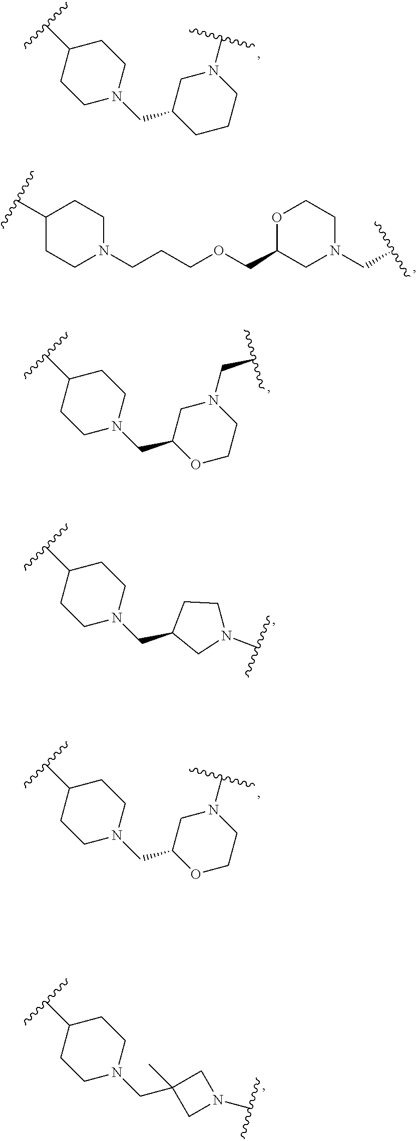

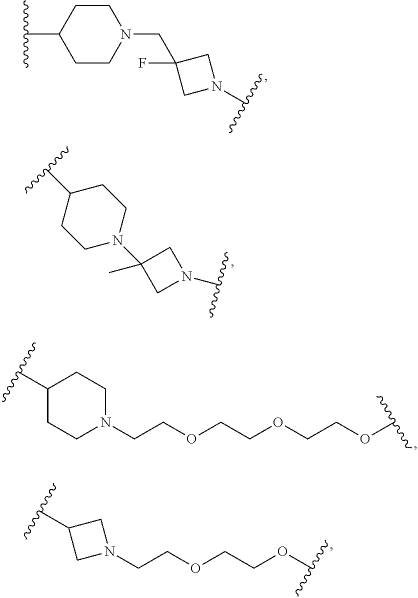 [SMILES: *C1CCN(CC2(F)CN(*)C2)CC1 ; *C1CCN(C2(C)CN(*)C2)CC1 ; *OCCOCCOCCN1CCC(*)CC1 ; *OCCOCCN1CC(*)C1]

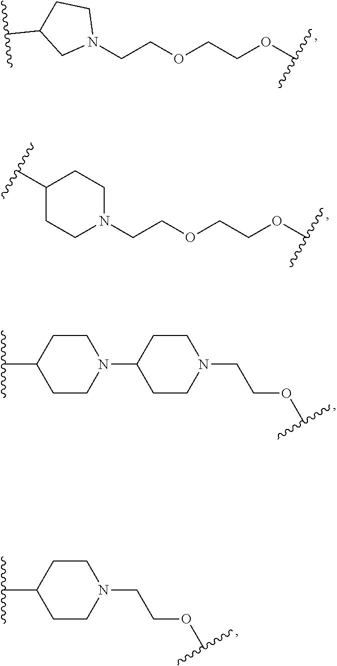 [SMILES: *OCCOCCN1CCC(*)C1 ; *OCCOCCN1CCC(*)CC1 ; *OCCN1CCC(N2CCC(*)CC2)CC1 ; *OCCN1CCC(*)CC1]

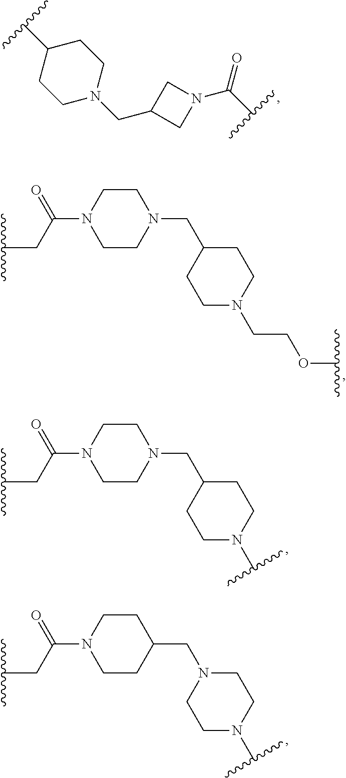 [SMILES: *C(=O)N1CC(CN2CCC(*)CC2)C1 ; *CC(=O)N1CCN(CC2CCN(CCO*)CC2)CC1 ; *CC(=O)N1CCN(CC2CCN(*)CC2)CC1 ; *CC(=O)N1CCC(CN2CCN(*)CC2)CC1]

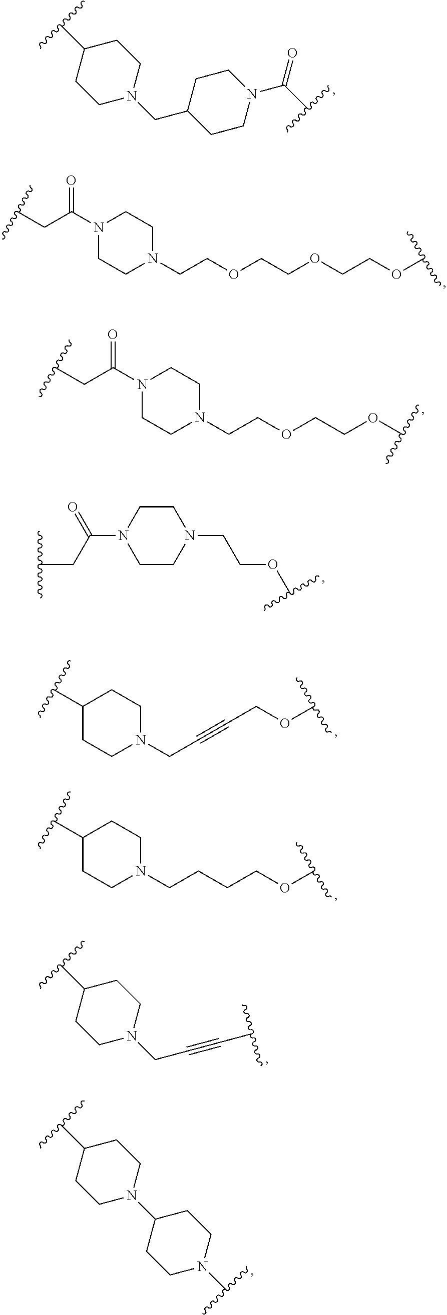

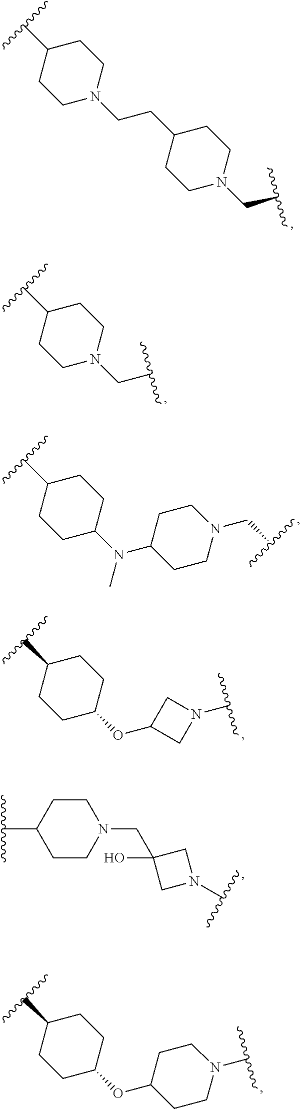

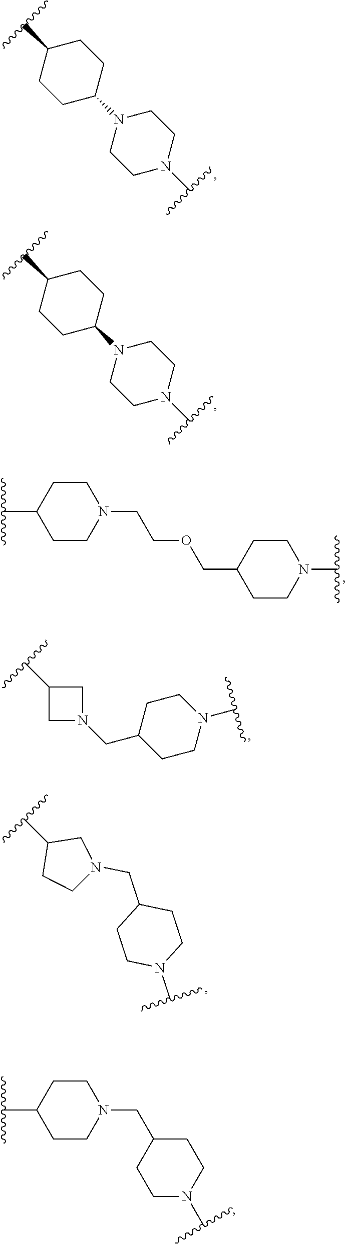 [SMILES: *N1CCN([C@H]2CC[C@H](*)CC2)CC1 ; *N1CCN([C@H]2CC[C@@H](*)CC2)CC1 ; *C1CCN(CCOCC2CCN(*)CC2)CC1 ; *C1CN(CC2CCN(*)CC2)C1 ; *C1CCN(CC2CCN(*)CC2)C1 ; *C1CCN(CC2CCN(*)CC2)CC1]

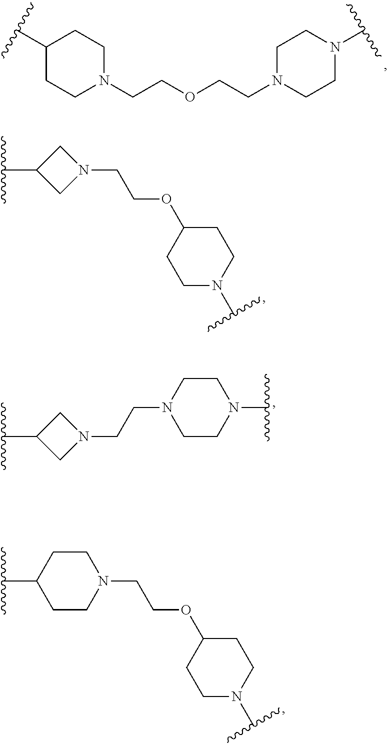 [SMILES: *C1CCN(CCOCCN2CCN(*)CC2)CC1 ; *C1CN(CCOC2CCN(*)CC2)C1 ; *C1CN(CCN2CCN(*)CC2)C1 ; *C1CCN(CCOC2CCN(*)CC2)CC1]

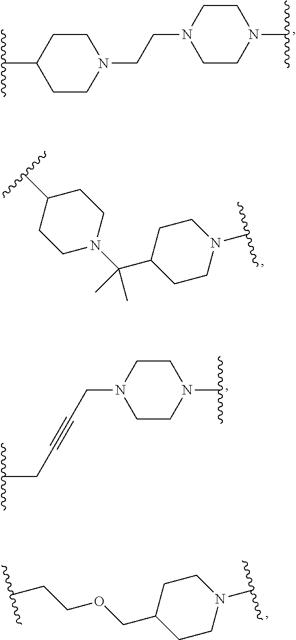 [SMILES: *C1CCN(CCN2CCN(*)CC2)CC1 ; *C1CCN(C(C)(C)C2CCN(*)CC2)CC1 ; *CC#CCN1CCN(*)CC1 ; *CCOCC1CCN(*)CC1]

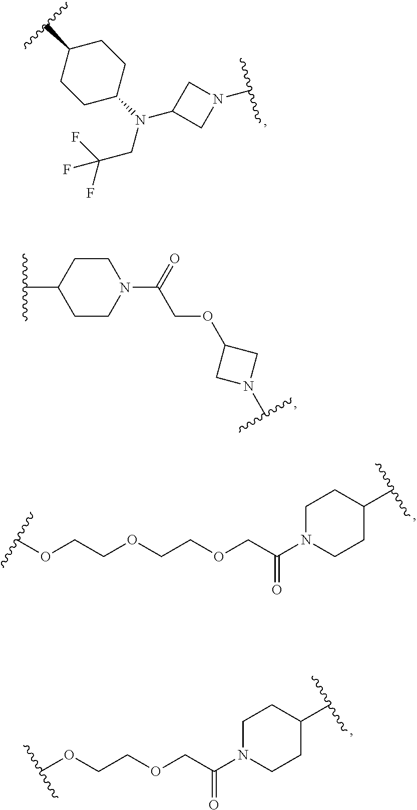 [SMILES: *N1CC(N(CC(F)(F)F)[C@H]2CC[C@H](*)CC2)C1 ; *C1CCN(C(=O)COC2CN(*)C2)CC1 ; *OCCOCCOCC(=O)N1CCC(*)CC1 ; *OCCOCC(=O)N1CCC(*)CC1]

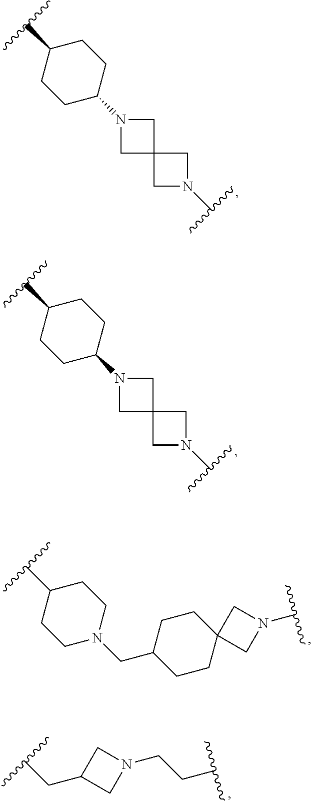 [SMILES: *N1CC2(C1)CN([C@H]1CC[C@H](*)CC1)C2 ; *N1CC2(C1)CN([C@H]1CC[C@@H](*)CC1)C2 ; *C1CCN(CC2CCC3(CC2)CN(*)C3)CC1 ; *CCN1CC(C*)C1]

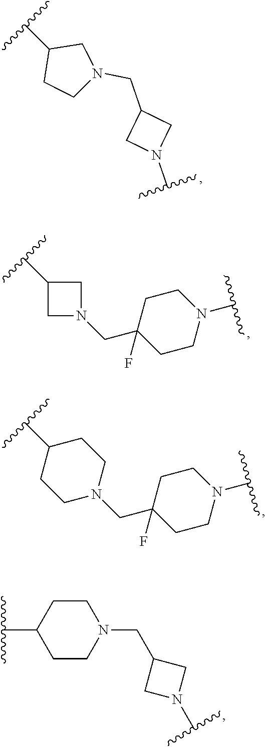 [SMILES: *C1CCN(CC2CN(*)C2)C1 ; *C1CN(CC2(F)CCN(*)CC2)C1 ; *C1CCN(CC2(F)CCN(*)CC2)CC1 ; *C1CCN(CC2CN(*)C2)CC1]

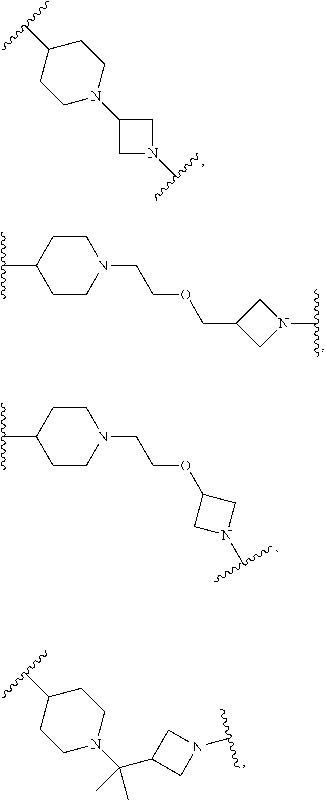 [SMILES: *C1CCN(C2CN(*)C2)CC1 ; *C1CCN(CCOCC2CN(*)C2)CC1 ; *C1CCN(CCOC2CN(*)C2)CC1 ; *C1CCN(C(C)(C)C2CN(*)C2)CC1]

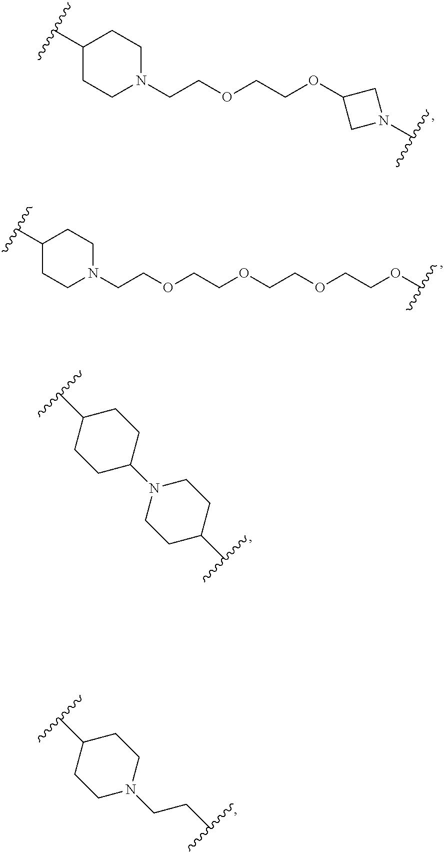 [SMILES: *C1CCN(CCOCCOC2CN(*)C2)CC1 ; *OCCOCCOCCOCCN1CCC(*)CC1 ; *C1CCC(N2CCC(*)CC2)CC1 ; *CCN1CCC(*)CC1]

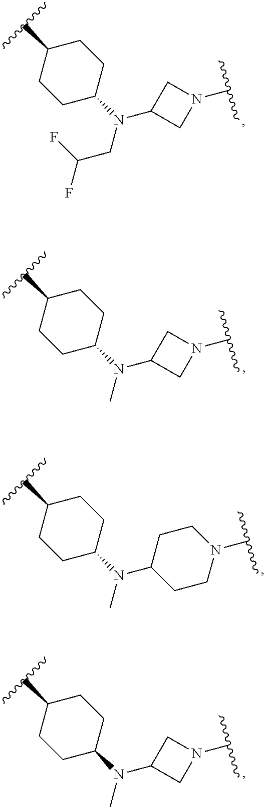 [SMILES: *N1CC(N(CC(F)F)[C@H]2CC[C@H](*)CC2)C1 ; *N1CC(N(C)[C@H]2CC[C@H](*)CC2)C1 ; *N1CCC(N(C)[C@H]2CC[C@H](*)CC2)CC1 ; *N1CC(N(C)[C@H]2CC[C@@H](*)CC2)C1]

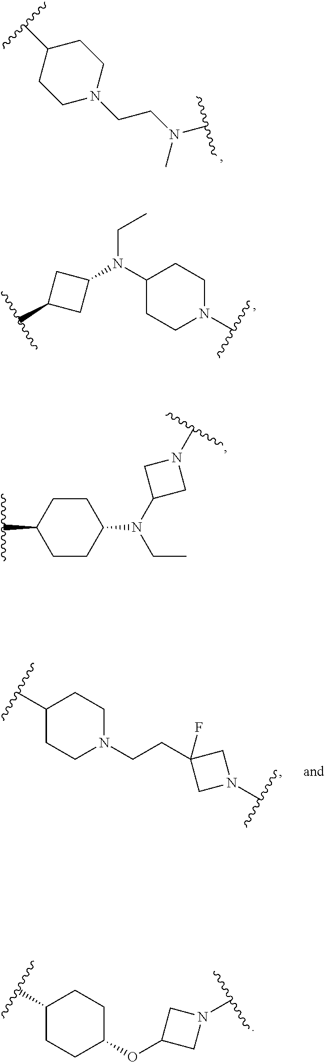 [SMILES: *C1CCN(CCN(*)C)CC1 ; *N1CCC(N(CC)[C@H]2C[C@H](*)C2)CC1 ; *N1CC(N(CC)[C@H]2CC[C@H](*)CC2)C1 ; *C1CCN(CCC2(F)CN(*)C2)CC1 ; *N1CC(O[C@H]2CC[C@@H](*)CC2)C1]

28. (canceled)

29. The bifunctional compound of claim 1, wherein the compound is selected from the compounds in any of Tables 1, 2, 3, 4, 5, 6, 7, 8, 9, 10, 11, 12, 13, 14, 15, 16, 17, and 18, and pharmaceutically acceptable salts thereof.

30. (canceled)

31. A pharmaceutical composition comprising the bifunctional compound of claim 1 and one or more pharmaceutically acceptable excipients.

32. The pharmaceutical composition of claim 31 further comprising an additional bioactive agent, wherein the bioactive agent is an anti-cancer agent, an anti-inflammatory agent, an anti-neurodegenerative agent, or an anti-immunological agent.

33. A method of treating a disease or disorder in a subject, the method comprising administering to the subject in need thereof a therapeutically effective amount of a bifunctional compound of claim 1.

34. The method of claim 33, wherein the disease or disorder is a neurodegenerative disease or disorder, an inflammatory disease or disorder, an immunological disease or disorder, and/or a cancer associated with signaling through signaling pathways regulated by IRAK-4 and/or the myddosome complex.

35-38. (canceled)
